(19) United States
(12) Patent Application Publication
(10) Pub. No.: US 2026/0335297 A1
(43) Pub. Date: Oct. 29, 2026

(54) COMBINATION THERAPIES INCLUDING METAL CHANNEL ACTIVATORS AND NMDA RECEPTOR ANTAGONISTS

(71) Applicant: Biohaven Therapeutics Ltd., New Haven, CT (US)

(72) Inventor: Vladimir CORIC, Madison, CT (US)

(21) Appl. No.: 19/105,812

(22) PCT Filed: Aug. 30, 2023
(86) PCT No.: PCT/US2023/073125

§ 371 Date: Feb. 23, 2025

(51) Int. Cl. A61K 31/4439; A61K 31/216; A61K 31/435; A61K 31/4402

(52) U.S. Cl. CPC A61K 31/4439; A61K 31/216; A61K 31/435; A61K 31/4402

(57) ABSTRACT

Provided is a pharmaceutical composition including a metal channel activator and an NMDA receptor antagonist. Also provided is a method of treating a depressive disorder, including administering the pharmaceutical composition. Also provided is a method of treating a neurological or neurodegenerative disorder, including administering the pharmaceutical composition. Also provided is a method of treating a pain disorder, including administering the pharmaceutical composition.

### CROSS-REFERENCE TO RELATED APPLICATIONS

This application is a national phase entry under 35 U.S.C. § 371 of International Application PCT/US2023/073125, filed Aug. 30, 2023, which claims priority to U.S. Provisional Patent Application No. 63/402,416, filed Aug. 30, 2022, all of which are herein incorporated by reference in their entirety.

### TECHNICAL FIELD

The present invention relates to combination therapies for treatment of various medical conditions. Specifically, the present invention relates to a combination of a metal channer activator and N-methyl-D-aspartate (NMDA) receptor antagonists for treatment of neurological and neurodegenerative disorders.

### BACKGROUND

Metal channel activators (openers) are associated with a wide range of physiological functions including the regulation of the electrical properties of excitable cells. Metal channels control the follow of metal ions such as Potassium (K⁺) and Sodium (Na⁺) across a cell membrane. A primary function of these channels in the brain is to regulate the neuronal action potential. Several neurologic disorders are potentially due to dysregulation of metal channels.

Potassium (K⁺) channels, present on the plasma membranes of most cell types, are the most diverse class of all ion channels. Potassium channels of the Kv7 family of voltage-gated potassium (K⁺) channels are of particular therapeutic interest due to their importance in neurological conditions such as excitability disorders including Amyotrophic lateral sclerosis (ALS). There are five members of the Kv7 family of voltage-gated potassium (K⁺) channels, including Kv7.1, Kv7.2, Kv7.3, Kv7.4, and Kv7.5.

Metal channel activators have been reported to be useful for treatment of various neurological and neurodegenerative disorders. Thus far, only one metal channel activator, Retigabine, has been FDA approved. Retigabine is used as anticonvulsant for the treatment of epilepsy. Further Kv7 channel activators have been proposed for the treatment of many conditions including substance abuse and mood disorders (Vigil F A, Carver C M, Shapiro M S. Pharmacological Manipulation of K v 7 Channels as a New Therapeutic Tool for Multiple Brain Disorders. Front Physiol. 2020 Jun. 19; 11:688. doi: 10.3389/fphys.2020.00688). There remains a need, however, for new therapies utilizing Kv7. One solution is combining metal channel activators with other therapeutic agents to significantly improve treatment outcomes.

NDMA receptor antagonists target the NDMA receptor, a receptor which allows calcium efflux in the presence of glutamate or glycine when neurons are depolarized. NDMA receptor antagonists prevent calcium efflux and have been used as anesthetics. Recent research, primarily focused on Ketamine, has revealed an unexpected antidepressant effect. Lanicemine has been developed as an alternative antidepressant to Ketamine with reduced side effects but has not successfully treated depression as a single agent in randomized controlled trials.

Described herein are compositions and methods for combination therapies of Kv7 openers with NMDA receptor antagonists. The combination therapies have treatment capabilities greater than Kv7 openers or NMDA receptor antagonists alone, and may be particularly useful for depressive disorders. Pharmaceutical compositions combining these agents may also be useful for treatment of a pain and neurological or neurodegenerative diseases. These two agents target distinct molecular targets associated with depressive disorders, pain disorders, neurological diseases and neurodegenerative disorders. Specifically, the Kv7 channel opener reduces excitability, and NMDA receptor antagonists reduce efflux of cations such as calcium. We have found that combining Kv7 opener and an NMDA receptor antagonist will yield improved treatment outcomes as described herein.

### SUMMARY

The present invention is directed to combination therapies including a metal channel activator and an NMDA receptor antagonist.

In an embodiment, provided is a pharmaceutical composition including a metal channel activator and an NMDA receptor antagonist.

In another embodiment, provided is a method of treating a depressive disorder, including administering the pharmaceutical composition.

In another embodiment, provided is a method of treating a neurological or neurodegenerative disorder, including administering the pharmaceutical composition.

In another embodiment, provided is a method of treating a pain disorder, including administering the pharmaceutical composition.

### DETAILED DESCRIPTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

As used herein, when specific definition is not otherwise provided, the term “substituted” refers to a group substituted with deuterium, a halogen (—F, —Cl, —Br, —I), a hydroxy group (—OH), an amino group (—NH₂), a carboxyl group (—CO₂H), a substituted or unsubstituted C₁-C₁₀ amine group, a nitro group (—NO₂), a C₁-C₁₀ alkyl group, a C₃-C₁₀ cycloalkyl group, a C₆-C₁₂ aryl group, a C₁-C₁₀ alkoxy group, a C₁ to C₁₀ trifluoroalkyl group such as a trifluoromethyl group (—CF₃) and the like, or a cyano group (—CN) instead of at least one hydrogen of a substituting group or compound.

Additional aspects will be set forth in part in the description which follows and, in part, will be apparent from the description.

As used herein, “modulator”, “modulators”, and “modulating”, when describing compounds, describe compounds that may enact their effect through a number of mechanisms of action including but not limited to: binding to the active site of a protein, binding to a region of the protein away from the active site, causing relocalization of a protein, inducing degradation of a protein, inducing stabilization of a protein, causing a conformational change in a protein, decreasing the activation threshold for a protein, increasing the activation threshold for a protein, altering posttranslational modifications for a protein, reducing the transcription of a gene, increasing the transcription of a gene, reducing the translation of an mRNA transcript, increasing the translation of an mRNA transcript, disrupting an interaction between two proteins, and stabilizing an interaction between two proteins; wherein the protein may be the target of modulation or an intermediary protein which is associated with modulation of the target protein. Modulators of the targets described herein (i.e., Kv7 and NMDA receptor) may each or both be small molecule compounds, proteins, antibody fragments or antibodies, or any other construct effecting modulation. It should be appreciated that an NMDA receptor antagonist is a type of modulator of the NMDA receptor.

As used herein, “NMDA receptor antagonist” or alternatively “NMDA modulator” means any compound (small molecule, peptide, etc.) effecting modulation or desired change in biological activity of the NMDA receptor. In various embodiments, NMDA receptor antagonists may target particular portions of an NMDA receptor, such as GluN1 (alternatively NR1) or GluN2 (alternatively NR2) subunits, or additionally or alternatively GluN3 subunits. In an embodiment, NMDA receptor antagonist target GluN1 subunits, in some cases selectively. In an embodiment, NMDA receptor antagonists target one or more GluN2 subtypes GluN2A, GluN2B, GluN2C, and/or GluN2D, in some cases selectively. In some embodiments, NMDA receptor antagonists may target one or more GluN3 subtypes GluN3A, GluN3B, and/or GluN3C, in some cases selectively. In some embodiments, NMDA receptor antagonists may target an allosteric site. In some embodiments, NMDA receptor antagonists may include partial agonists which exert dual (agonist/antagonist) effects on NMDA receptors. In certain embodiments, NMDA receptor antagonists may include agents which have a target other than the NMDA receptor which result in an NMDA receptor antagonist-like physiologic effect.

As used herein, “neurological disease” refers to a disease or disorder which affects the brain and/or nerves found elsewhere in the body. Such neurologic diseases may include: Absence of the Septum Pellucidum, Acid Lipase Disease, Acute Disseminated Encephalomyelitis, Adrenoleukodystrophy, Agenesis of the Corpus Callosum, Agnosia, Aicardi Syndrome, Aicardi-Goutieres Syndrome, Alexander Disease, Alpers' Disease, Alternating Hemiplegia, Alzheimer's Disease, Amyotrophic Lateral Sclerosis (ALS), Anencephaly, Angelman Syndrome, Antiphospholipid Syndrome, Aphasia, Apraxia, Arachnoid Cysts, Arachnoiditis, Arteriovenous Malformation, Asperger Syndrome, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Back Pain, Barth Syndrome, Batten Disease, Behcet's Disease, Bell's Palsy, Benign Essential Blepharospasm, Binswanger's Disease, Brachial Plexus Injuries, Brain and Spinal Tumors, Brown-Sequard Syndrome, CADASIL, Canavan Disease, Carpal Tunnel Syndrome, Central Cord Syndrome, Central Pain Syndrome, Central Pontine Myelinolysis, Cephalic Disorders, Cerebellar Degeneration, Cerebellar Hypoplasia, Cerebral Aneurysms, Cerebral Arteriosclerosis, Cerebral Atrophy, Cerebral Cavernous Malformation, Cerebral Hypoxia, Cerebral Palsy, Cerebro-Oculo-Facio-Skeletal Syndrome (COFS), Charcot-Marie-Tooth Disease, Chiari Malformation, Chorea, Chronic Inflammatory Demyelinating Polyneuropathy (CIDP), Chronic Pain, Coffin Lowry Syndrome, Colpocephaly, Coma, Complex Regional Pain Syndrome, Congenital Myasthenia, Congenital Myopathy, Corticobasal Degeneration, Craniosynostosis, Creutzfeldt-Jakob Disease, Cushing's Syndrome, Dandy-Walker Syndrome, Deep Brain Stimulation for Movement Disorders, Dementia, Dementia With Lewy Bodies, Dermatomyositis, Developmental Dyspraxia, Diabetic Neuropathy, Dravet Syndrome, Dysautonomia, Dysgraphia, Dyslexia, Dyssynergia Cerebellaris Myoclonica, Dystonia, Empty Sella Syndrome, Encephalitis Lethargica, Encephaloceles, Encephalopathy, Epilepsy, Erb-Duchenne and Dejerine-Klumpke Palsies, Essential Tremor, Fabry Disease, Fahr's Syndrome, Familial Periodic Paralyses, Farber's Disease, Febrile Seizures, Fibromuscular Dysplasia, Foot Drop, Friedreich Ataxia, Frontotemporal Dementia, Functional Neurologic Disorder, Gaucher Disease, Generalized Gangliosidoses, Gerstmann's Syndrome, Gerstmann-Straussler-Scheinker Disease, Giant Axonal Neuropathy, Glossopharyngeal Neuralgia, Guillain-Barré Syndrome, Headache, Hemicrania Continua, Hemifacial Spasm, Hereditary Neuropathies, Hereditary Spastic Paraplegia, Herpes Zoster Oticus, Holmes-Adie Syndrome, Holoprosencephaly, Huntington's Disease, Hydranencephaly, Hydrocephalus, Hydromyelia, Hypersomnia, Hypertonia, Hypotonia, Inclusion Body Myositis, Incontinentia Pigmenti, Infantile Neuroaxonal Dystrophy, Infantile Refsum Disease, Infantile Spasms, Inflammatory Myopathies, Iniencephaly, Isaacs' Syndrome, Joubert Syndrome, Kearns-Sayre Syndrome, Kennedy's Disease, Kleine-Levin Syndrome, Klippel-Feil Syndrome, Klippel-Trenaunay Syndrome (KTS), Klüver-Bucy Syndrome, Krabbe Disease, Kuru, Lambert-Eaton Myasthenic Syndrome, Landau-Kleffner Syndrome, Learning Disabilities, Leigh's Disease, Lennox-Gastaut Syndrome, Lesch-Nyhan Syndrome, Leukodystrophy, Lipid Storage Diseases, Lipoid Proteinosis, Lissencephaly, Locked-In Syndrome, Machado-Joseph Disease and Spinocerebellar Ataxia, Megalencephaly, Melkersson-Rosenthal Syndrome, Meningitis and Encephalitis, Menkes Disease, Meralgia Paresthetica, Metachromatic Leukodystrophy, Microcephaly, Migraine, Miller Fisher Syndrome, Mitochondrial Myopathies, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia Gravis, Myoclonus, Myopathy, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Sequelae Of Lupus, Neuromyelitis Optica, Neuronal Migration Disorders, Neurosarcoidosis, Neurosyphilis, Neurotoxicity, Niemann-Pick Disease, Normal Pressure Hydrocephalus, Occipital Neuralgia, Ohtahara Syndrome, Olivopontocerebellar Atrophy, Opsoclonus Myoclonus, Orthostatic Hypotension, Paraneoplastic Syndromes, Paresthesia, Parkinson's Disease, Paroxysmal Choreoathetosis, Paroxysmal Hemicrania, Parry-Romberg Syndrome, Pelizaeus-Merzbacher Disease, Peripheral Neuropathy, Periventricular Leukomalacia, Pervasive Developmental Disorders, Pinched Nerve, Piriformis Syndrome, Pituitary Tumors, Polymyositis, Pompe Disease, Porencephaly, Post-Polio Syndrome, Postural Tachycardia Syndrome, Primary Lateral Sclerosis, Progressive Multifocal Leukoencephalopathy, Progressive Supranuclear Palsy, Prosopagnosia, Pseudotumor Cerebri, Rasmussen's Encephalitis, Refsum Disease, Repetitive Motion Disorders, Restless Legs Syndrome, Rett Syndrome, Reye's Syndrome, Sandhoff Disease, Schilder's Disease, Schizencephaly, Septo-Optic Dysplasia, Shaken Baby Syndrome, Shingles, Sjögren's Syndrome, Sleep Apnea, Sotos Syndrome, Spasticity, Spina Bifida, Spinal Cord Infarction, Spinal Cord Injury, Spinal Muscular Atrophy, Stiff-Person Syndrome, Striatonigral Degeneration, Stroke, Sturge-Weber Syndrome, Subacute Sclerosing Panencephalitis, SUNCT Headache, Swallowing Disorders, Sydenham Chorea, Syncope, Syringomyelia, Tabes Dorsalis, Tardive Dyskinesia, Tarlov Cysts, Tay-Sachs Disease, Tethered Spinal Cord Syndrome, Thoracic Outlet Syndrome, Thyrotoxic Myopathy, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis Syndromes of the Central and Peripheral Nervous Systems, Von Hippel-Lindau Disease (VHL), Wallenberg's Syndrome, Wernicke-Korsakoff Syndrome, Whiplash, Whipple's Disease, Williams Syndrome, Wilson Disease, and Zellweger Syndrome.

As used herein, the term “metal channel activator” is construed to include both metal channel activator and pharmaceutically acceptable salt thereof.

As used herein, the term “NMDA receptor antagonist” or “NMDA modulator” is construed to include both NMDA receptor antagonist and pharmaceutically acceptable salt thereof.

As used herein, the term “AUC” is the definite integral of the concentration of a drug in blood, cerebrospinal fluid, target organ, or any other physiologically relevant site after a dose is given as a function of time. AUC in this context is used to measure the total exposure to the drug across time. AUC can be evaluated over a definite time interval or estimated based on the integral drug concentrations measured over a time interval extrapolated to infinite time.

As used herein, the term “Cmax” is the highest concentration of a drug in the blood, cerebrospinal fluid, target organ, or any other physiologically relevant site after a dose is given.

As used herein, the term “Tmax” is the time taken after administration for a drug to reach its highest concentration in the blood, cerebrospinal fluid, target organ, or any other physiologically relevant site after a dose is given.

As used herein, the term “proteinopathies” refers to diseases characterized by an accumulation or aggregation of a single protein, or multiple proteins. Proteinopathies include but are not limited to: Creutzfeldt-Jakob disease and other prion diseases, Alzheimer's disease, Parkinson's disease, amyloidosis, multiple system atrophy, Amyotrophic lateral sclerosis (ALS), Frontotemporal Lobar Degeneration, and Lewy Body Dementia.

Additional aspects will be set forth in part of the description which follows and, in part, will be apparent from the description.

In the formulas set forth below, it is set out with substituents and definitions, and it should be clear to the reader that substituents and definitions are numbered (e.g., R1, R2, Y, etc.) and are intended to apply within a given formula. Numberings that are repeated across formulas are intentional and should be each read within the context of each particular formula.

### Metal Channel Activator

Examples of metal channel activators including Kv7 channel activators are disclosed in Formulas 1-171, the “Further embodiments” section, and in the corresponding referenced applications.

### Formula 1

In an embodiment, the Kv7 channel activator may be selected from one of the following compounds according to Formula 1. Such compounds are described in U.S. Pat. No. 9,481,653, issued Nov. 1, 2016, and corresponding to U.S. application Ser. No. 14/853,815 filed Sep. 14, 2015; and US Publication No 20210188782A1, published Jun. 24, 2021, and corresponding to U.S. application Ser. No. 17/127,231 filed Dec. 18, 2020; which are incorporated by reference in their entirety herein. In the case of any conflict of terminology in the context of Formula 1, these references incorporated by reference herein control.

In an embodiment, the Kv7 channel activator is a compound according to formula 1:

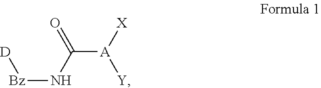

wherein D is optionally substituted C₃₋₆ carbocyclyl, optionally substituted C₂₋₅ heterocyclyl, isopropyl, or t-butyl; Bz is optionally substituted benzoimidazol-1,2-diyl; A is C₁₋₈ alkyl; X is F; and Y is H, F, Cl, Br, I, or a moiety having a molecular weight of 15 Da to 300 Da and consisting of 2 to 5 chemical elements, wherein the chemical elements are independently C, H, O, N, S, F, Cl, or Br.

In further embodiments, D is optionally substituted cyclobutyl, optionally substituted phenyl, optionally substituted isoxazolyl, optionally substituted pyridinyl, isopropyl, or t-butyl.

In further embodiments, D is optionally substituted cyclobutyl, optionally substituted phenyl, optionally substituted isoxazolyl, optionally substituted pyridinyl, isopropyl, or t-butyl, wherein each substituent of D and Y, if present, independently has a molecular weight of 15 Da to 200 Da and consists of 2 to 5 chemical elements, wherein the chemical elements are independently C, H, O, N, S, F, Cl, or Br.

In another embodiment, the Kv7 channel activator is a compound according to formula 1 wherein, D is optionally substituted C₃₋₆ carbocyclyl, optionally substituted C₂₋₅ heterocyclyl, isopropyl, or t-butyl; Bz is optionally substituted benzoimidazol-1,2-diyl; A is C₁₋₈ alkyl; X is H, F, CF₃, optionally substituted phenyl, or optionally substituted pyridinyl; and Y is H, F, Cl, Br, I, or a moiety having a molecular weight of 15 Da to 300 Da and consisting of 2 to 5 chemical elements, wherein the chemical elements are independently C, H, O, N, S, F, Cl, or Br.

In further embodiments, D is optionally substituted cyclobutyl, optionally substituted phenyl, optionally substituted isoxazolyl, optionally substituted pyridinyl, isopropyl, or t-butyl.

In further embodiments, each substituent of D, X, and Y, if present, independently has a molecular weight of 15 Da to 200 Da and consists of 2 to 5 chemical elements, wherein the chemical elements are independently C, H, O, N, S, F, Cl, or Br.

In further embodiments, R¹ is H, Cl, Br, CN, OCH₃, CF₃, —CO₂CH₂CH₃, C₁₋₄ alkyl, or C₁₋₄ hydroxyalkyl.

In further embodiments, X is optionally substituted phenyl.

In further embodiments, X is CF₃.

In further embodiments, X is optionally substituted pyridinyl.

In further embodiments, X is H.

In further embodiments, Y is H.

In further embodiments, Y is OH.

In further embodiments, the Kv7 channel activator is a compound selected from the

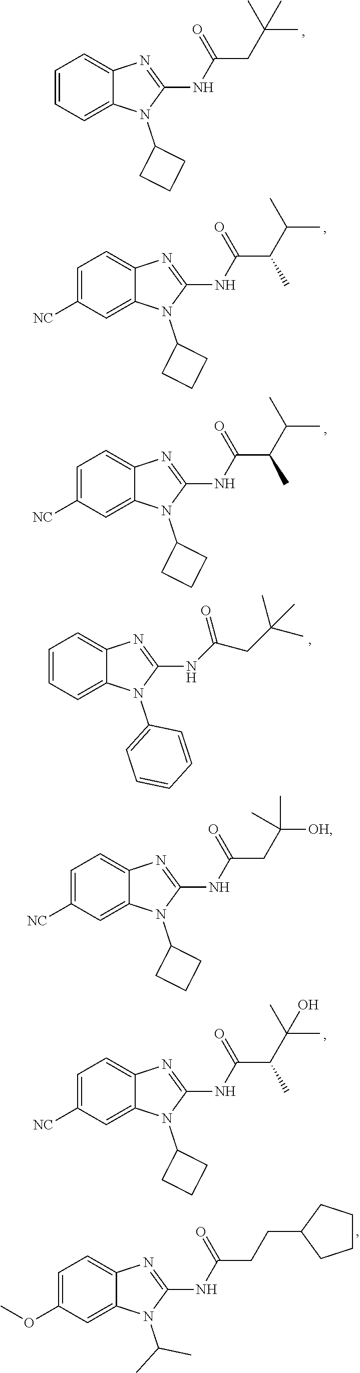

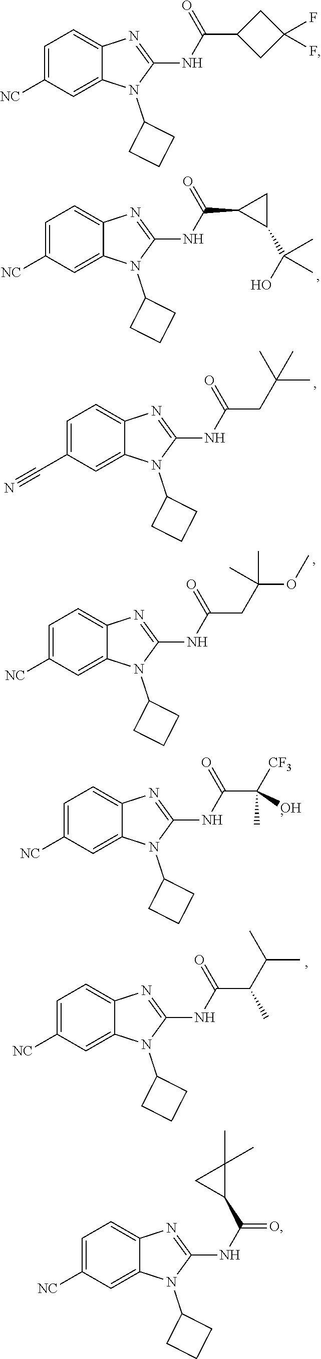

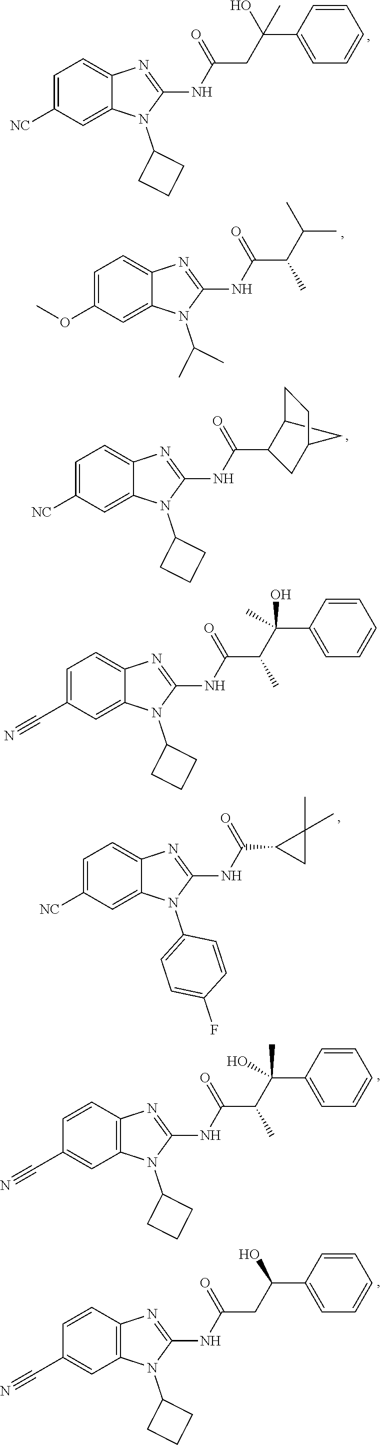

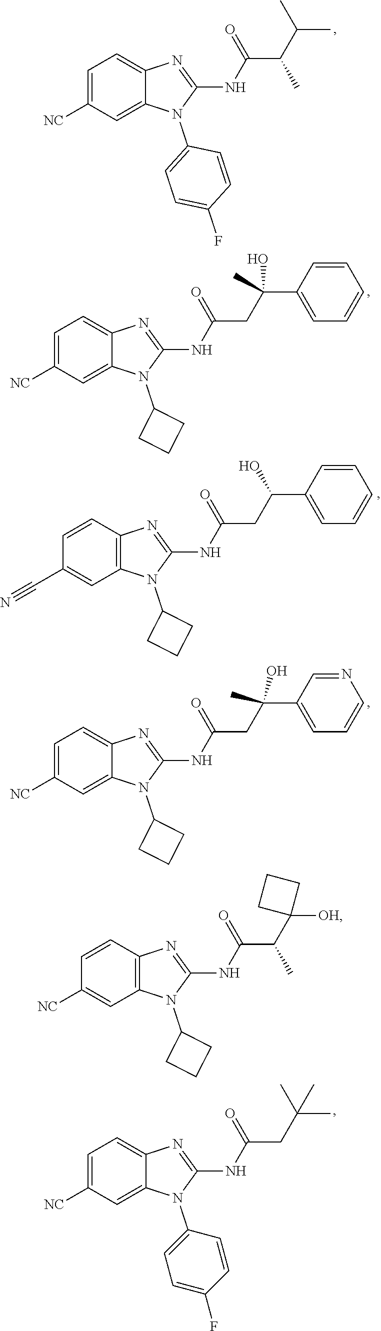

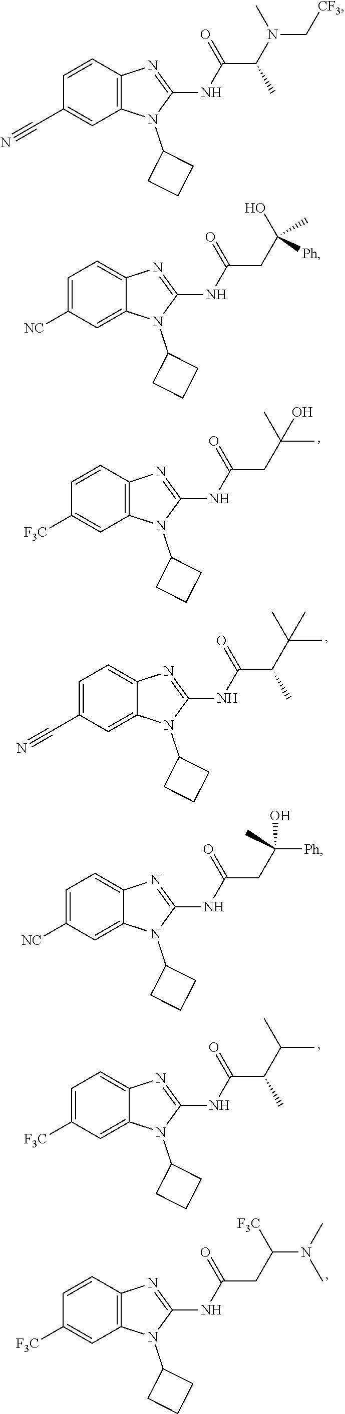

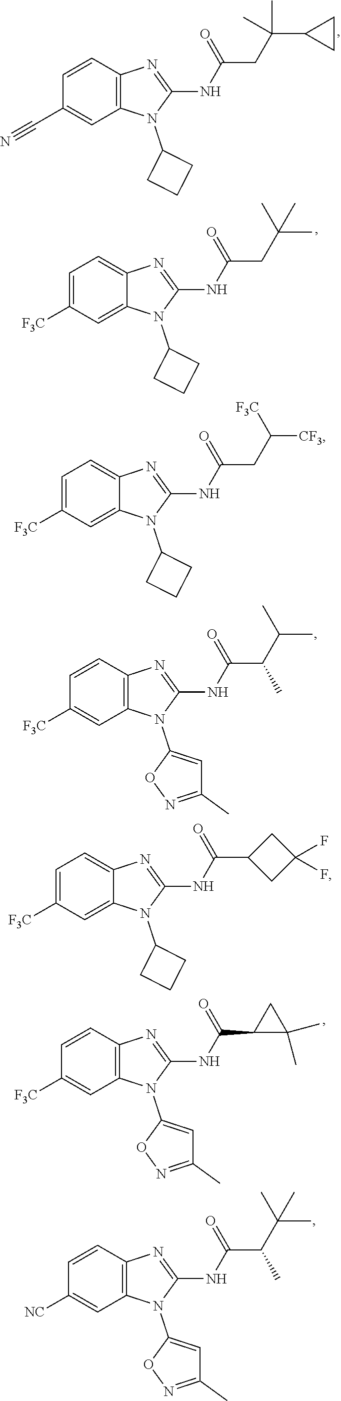

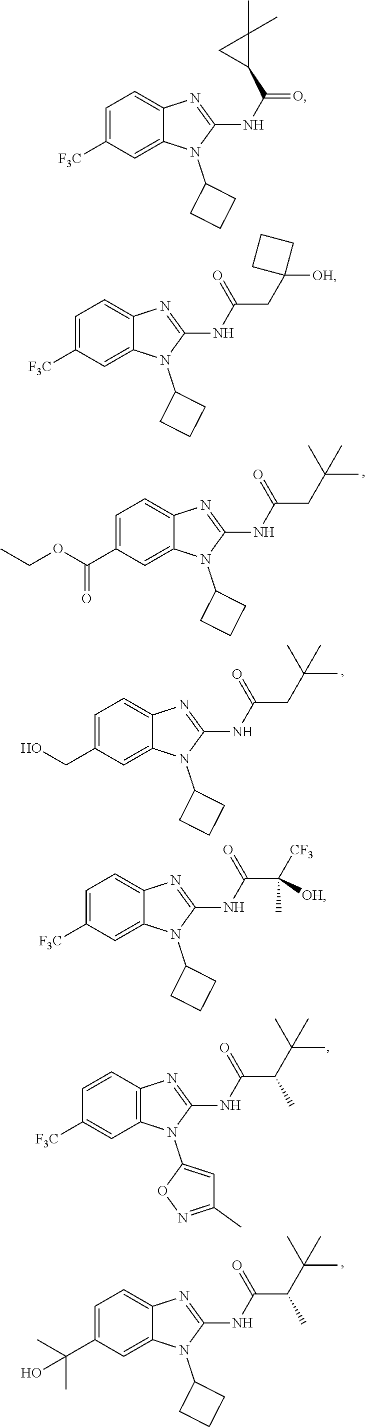

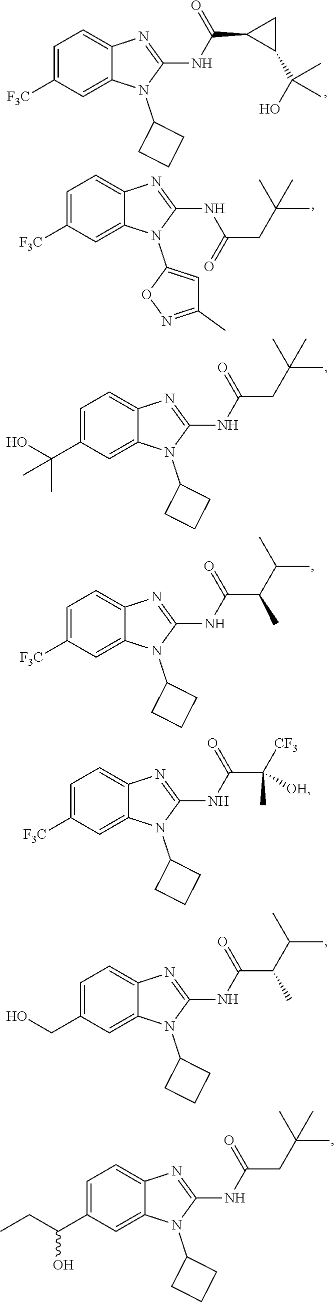

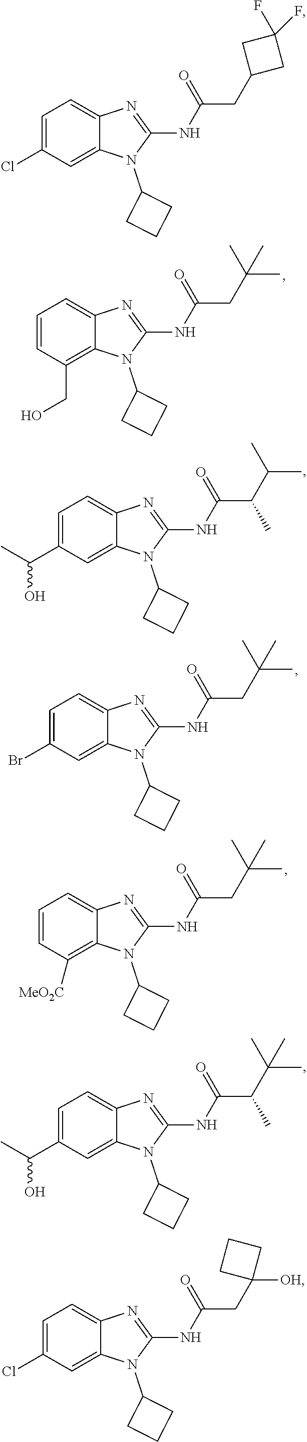

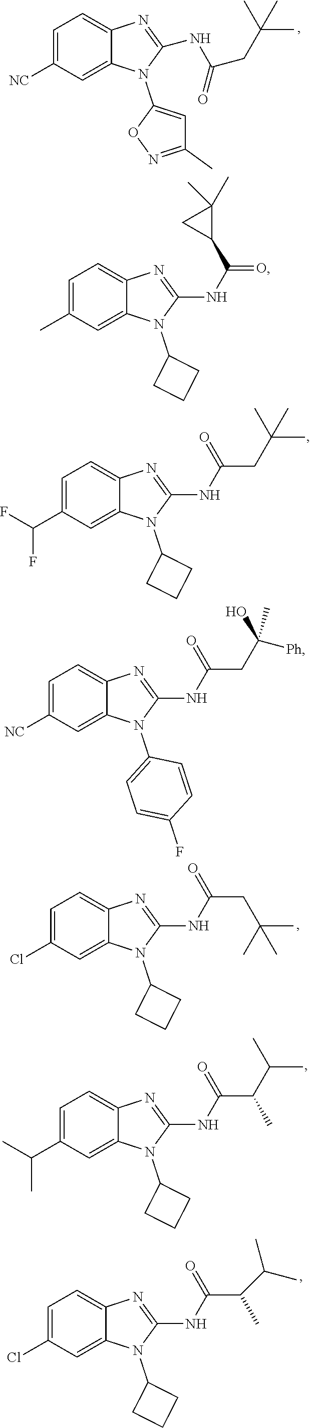

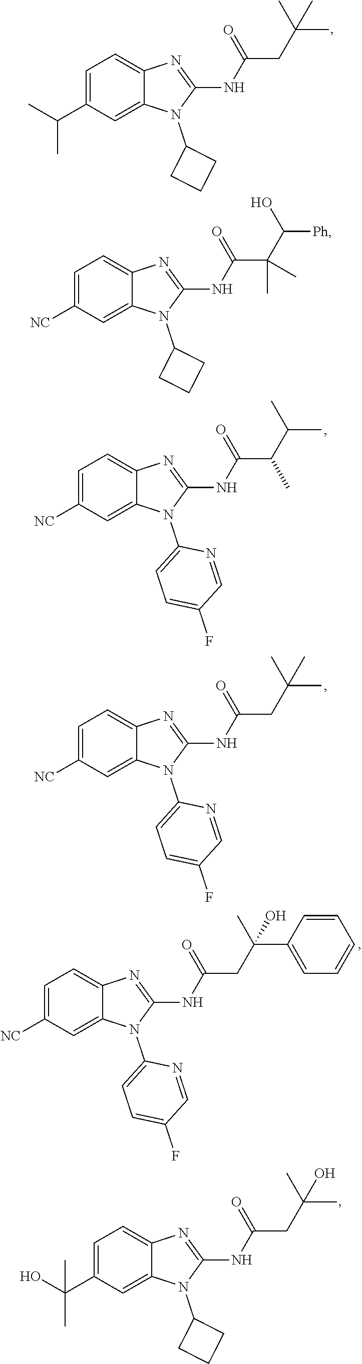

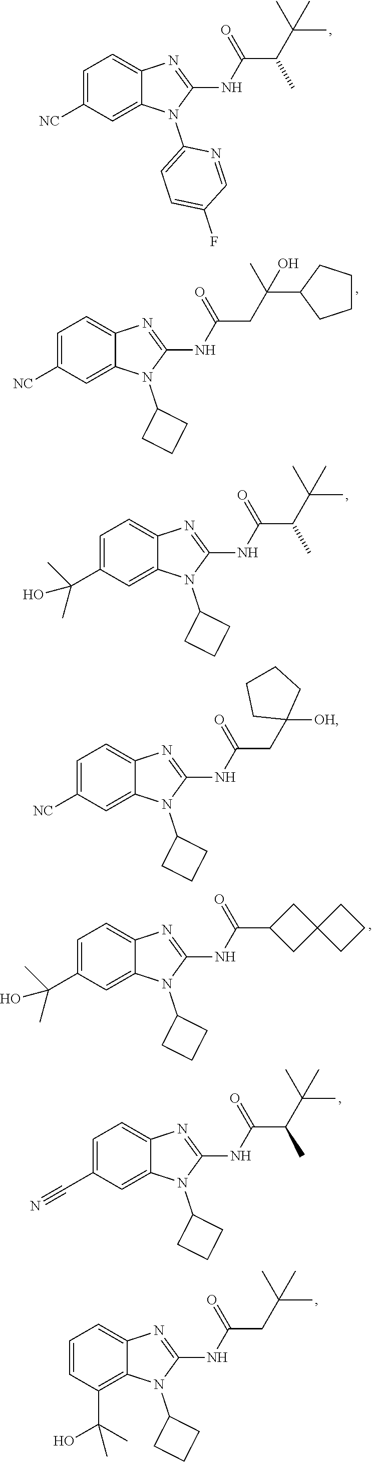

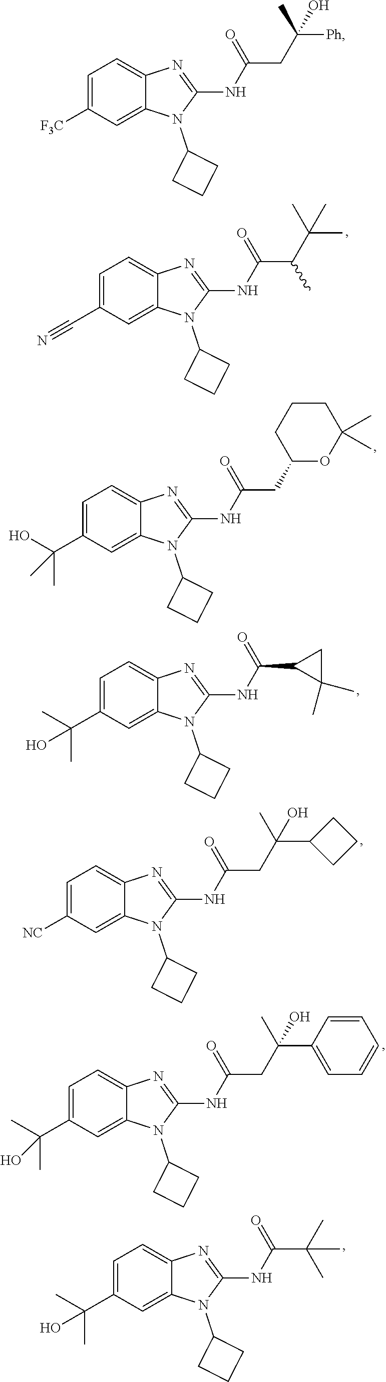

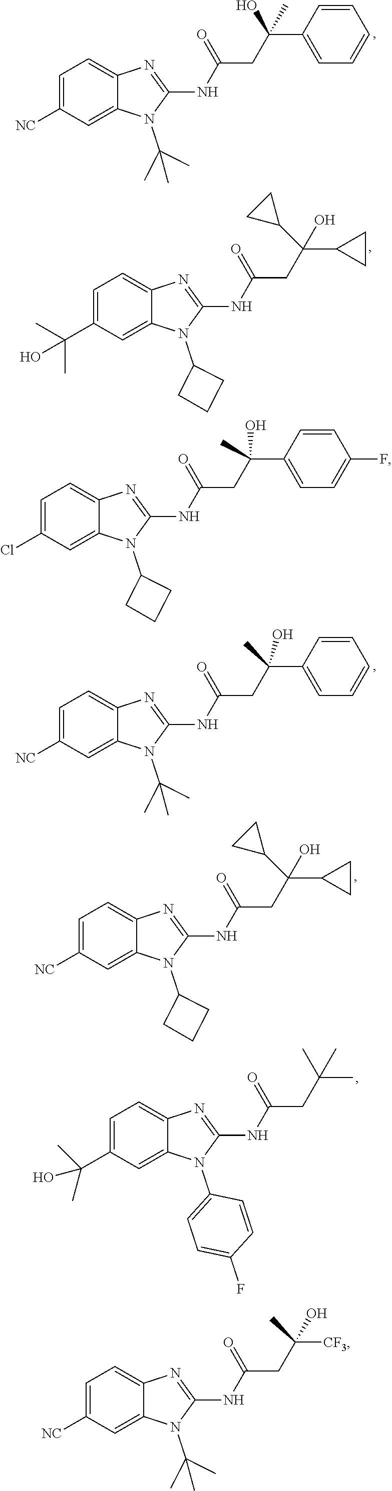

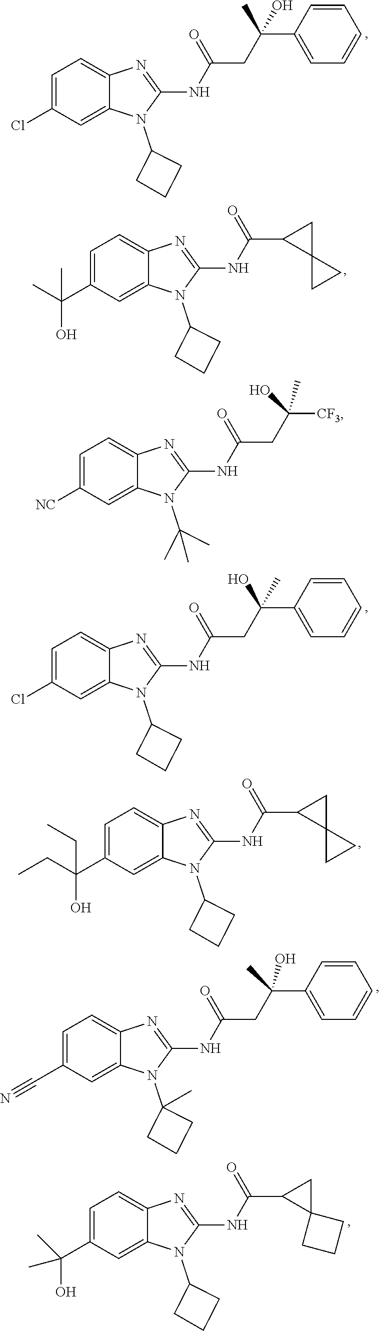

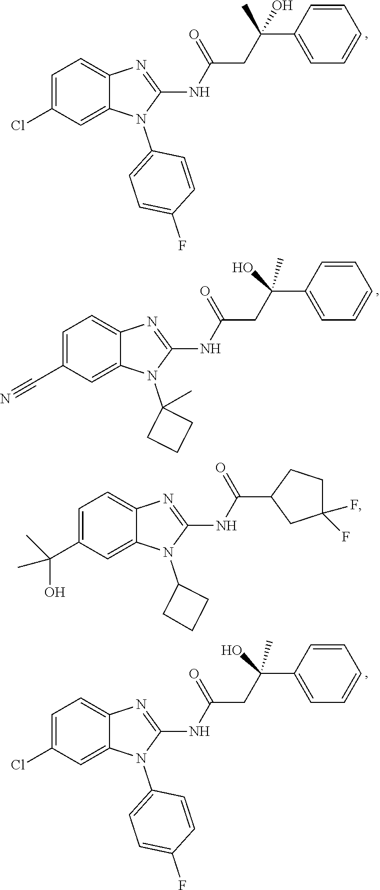

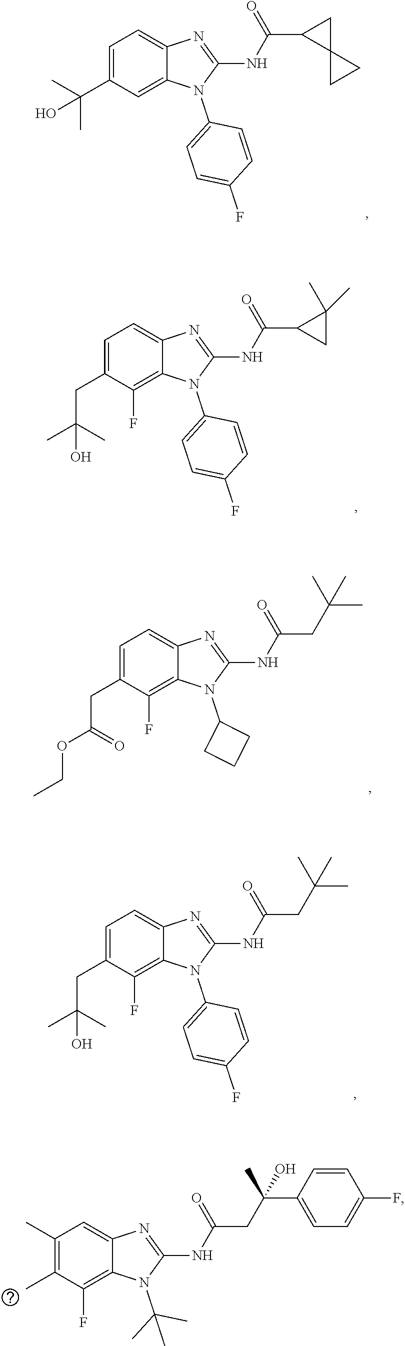

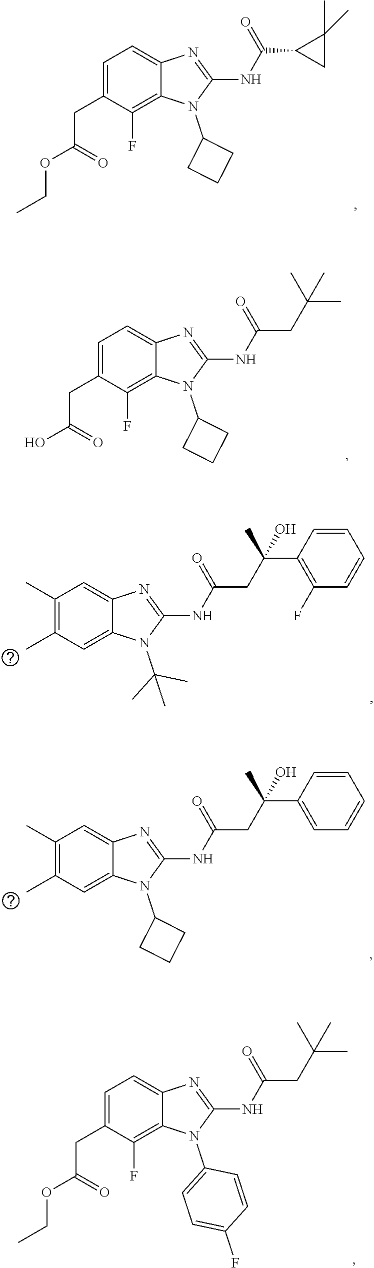

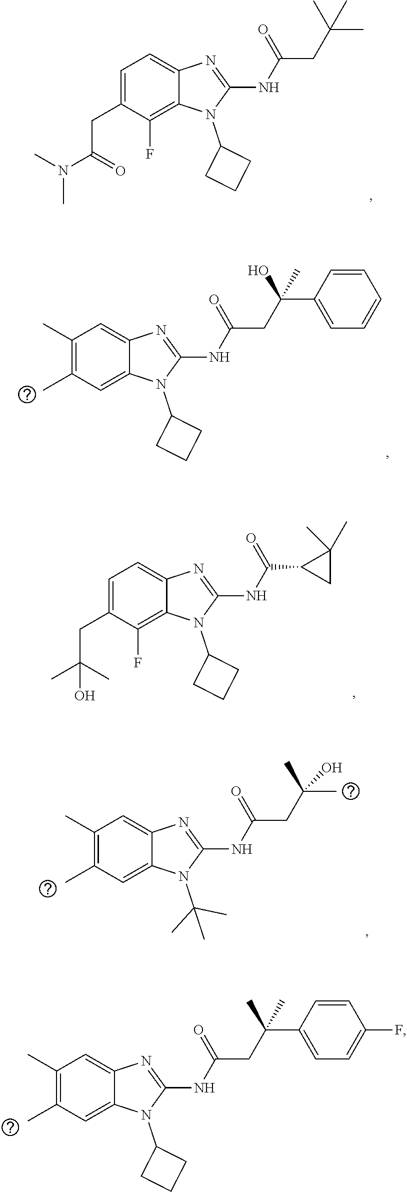

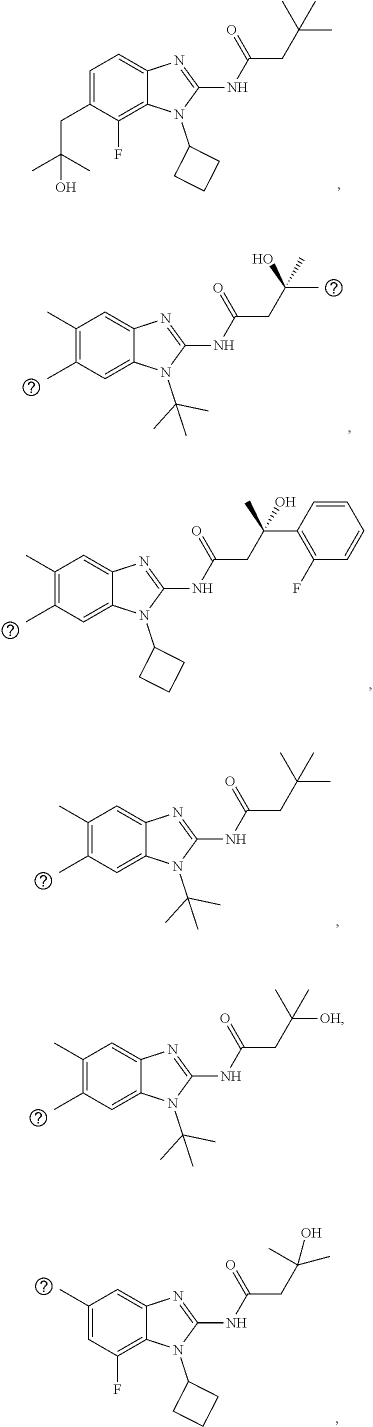

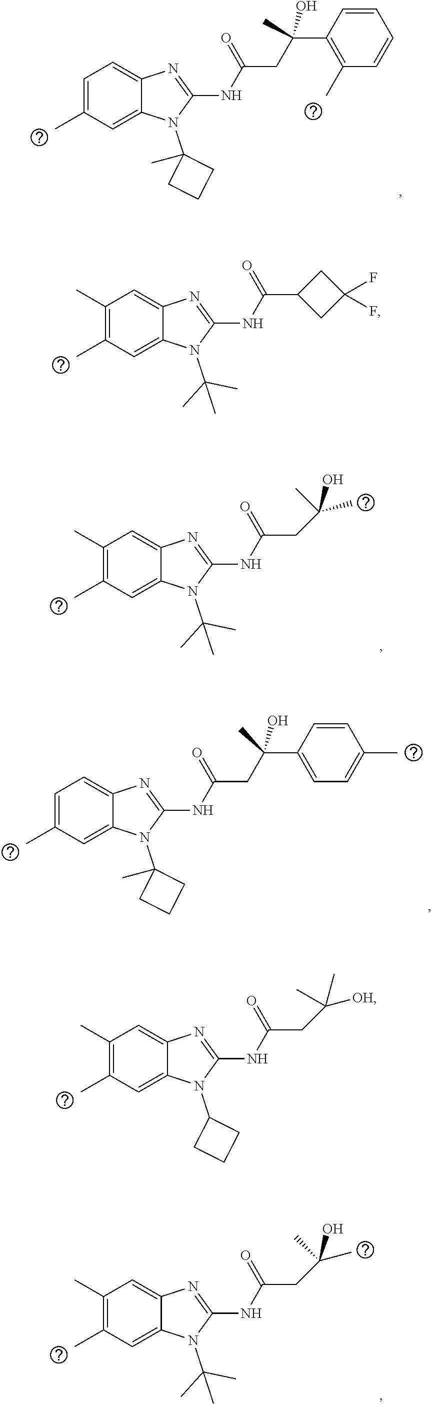

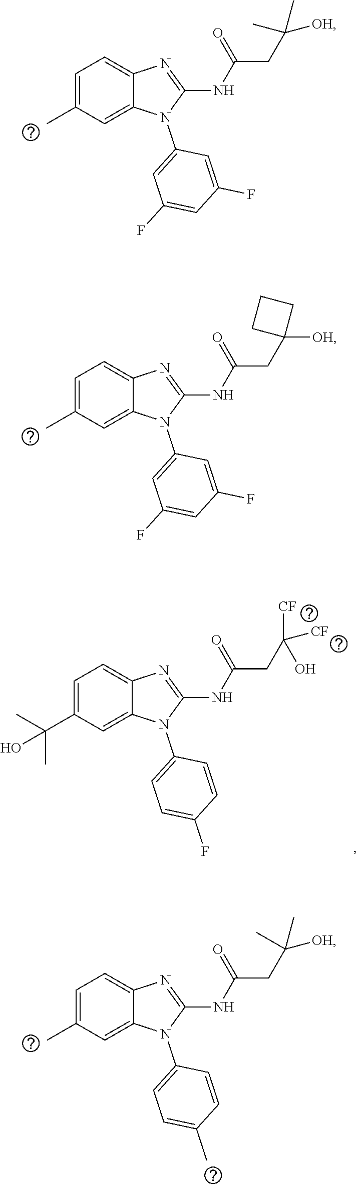

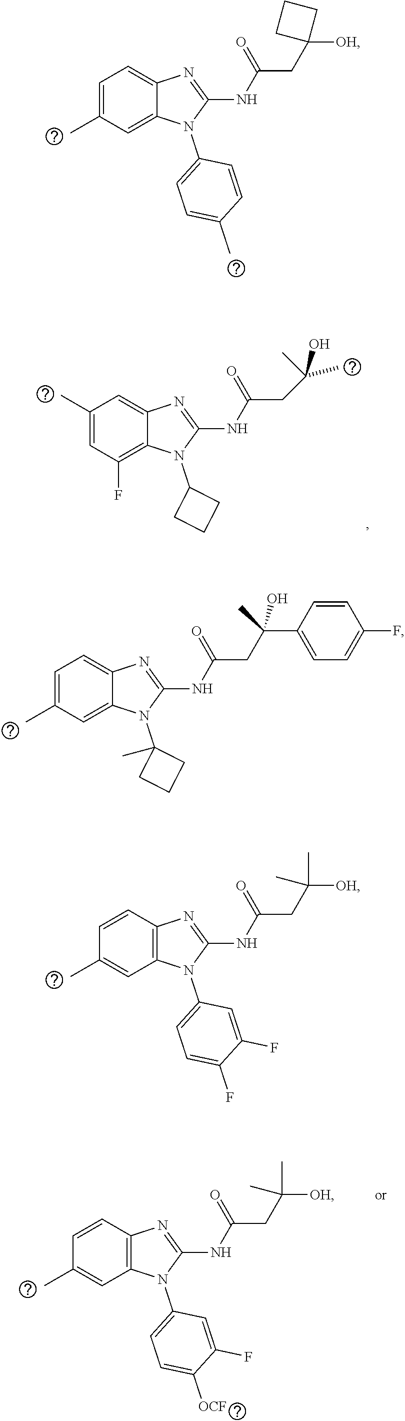

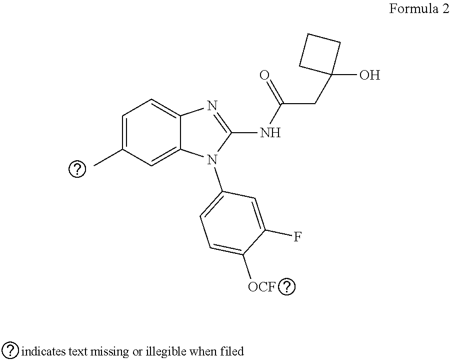

In an embodiment, the Kv7 channel activator may be selected from one of the following compounds according to Formula 2. Such compounds are described in U.S. Pat. No. 9,481,653 issued Nov. 1, 2016 and corresponding to US Application No. U.S. Ser. No. 14/853,815 filed Sep. 14, 2015; U.S. Pat. No. 9,914,708, issued Mar. 13, 2018 and corresponding to U.S. application Ser. No. 15/339,590 filed October 31; U.S. Pat. No. 10,385,025, issued Aug. 20, 2019 and corresponding to U.S. application Ser. No. 15/879,792 filed Jan. 25, 2018; U.S. Pat. No. 10,906,877 issued on Feb. 2, 2021 and corresponding to U.S. application Ser. No. 16/460,449 filed Jul. 2, 2019; U.S. Pat. No. 10,851,067 issued on Dec. 1, 2020 and corresponding to U.S. application Ser. No. 16/358,642 filed Mar. 19, 2019; U.S. Pat. No. 11,261,162 issued on Mar. 1, 2022 and corresponding to U.S. application Ser. No. 17/077,068 filed Oct. 22, 2020; US Publication No. 20210188782A1, published Jun. 24, 2021 and corresponding to U.S. application Ser. No. 17/127,231 filed Dec. 18, 2020; which are incorporated by reference in their entirety herein. In the case of any conflict of terminology in the context of Formula 2, these references incorporated by reference herein control.

In an embodiment, the Kv7 channel activator is a compound according to formula 2:

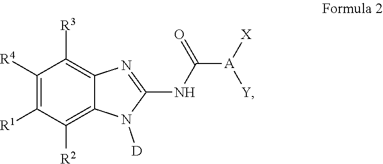

wherein D is optionally substituted cyclobutyl, optionally substituted phenyl, optionally substituted isoxazolyl, optionally substituted pyridinyl, isopropyl, or t-butyl; A is C₂₋₈ alkyl; X is H, F, CF₃ optionally substituted phenyl, or optionally substituted pyridinyl; Y is H, F, Cl, Br, I, or a moiety having a molecular weight of 15 Da to 300 Da and consisting of 2 to 5 chemical elements, wherein the chemical elements are independently C, H, O, N, S, F, Cl, or Br; R¹ is F, Cl, Br, CN, OCH₃, CHF₂, CF₃, C₁₋₄—CO₂-alkyl, C₁₋₄ alkyl, —CH₂CO₂H, —CH₂CO₂CH₂CH₃ or —CH₂CON(CH₃)₂, or C₁₋₅ hydroxyalkyl; and R², R³, and R⁴ are independently H, F, Cl, Br, I, or a substituent having a molecular weight of 15 Da to 200 Da and consisting of 2 to 5 chemical elements, wherein the chemical elements are independently C, H, O, N, S, F, Cl, or Br.

In further embodiments, Y is H, F, CF₃, OH, C₁₋₅ O-alkyl, C₀₋₆ alkylamino, optionally substituted tetrahydropyranyl, or C₀₋₆ fluoroalkylamino.

In further embodiments R¹ is Cl, Br, —OCH₃, —CN, —CF₃, —CH₂OH, —COOCH₂CH₃, —C(CH₃)₂OH, —CHOHCH₂CH₃, —CHOHCH₃, —CHF₂, —CH(CH₃)₂, —C(CH₂CH₃)₂OH, —CH₂COOCH₂CH₃, —CH₂C(CH₃)₂OH, —CH₂COOH, or —CH₂CON(CH₃)₂.

In further embodiments, R² is H, F, —CH₂OH, —CO₂Me, or —C(CH₃)₂OH.

In further embodiments, R³ is H.

In further embodiments, R⁴ is H, —CH₃, or —CF₃.

In further embodiments, R¹ is Cl, Br, CN, OCH₃, CF₃, —CO₂CH₂CH₃, C₁₋₄ alkyl, or C₁₋₄ hydroxyalkyl.

In further embodiments, X is optionally substituted phenyl.

In further embodiments, X is CF₃.

In further embodiments, X is optionally substituted pyridinyl.

In further embodiments, X is H.

In further embodiments, Y is H.

In further embodiments, Y is OH.

In further embodiments, the Kv7 channel activator is a compound selected from the

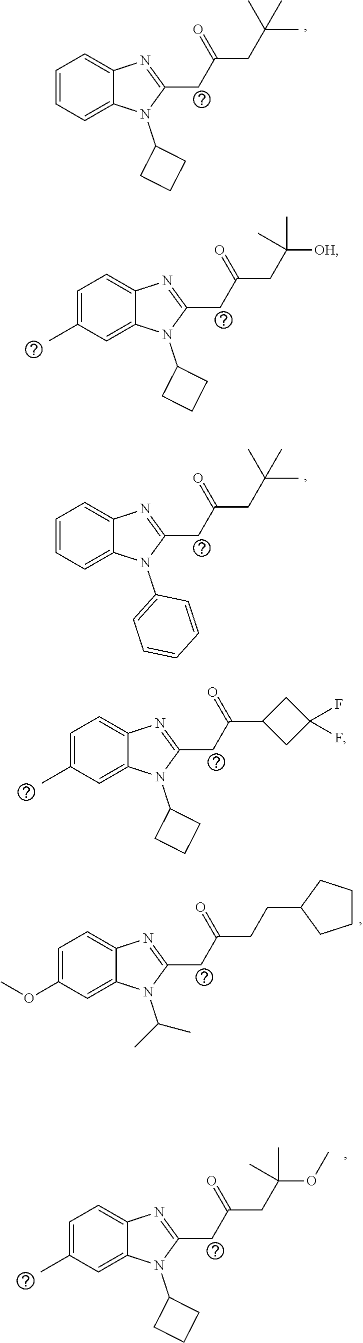

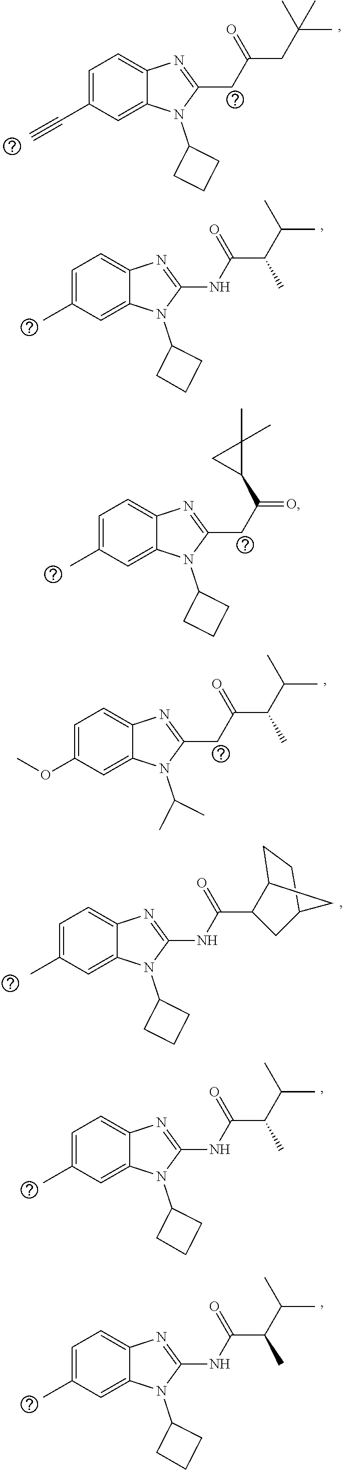

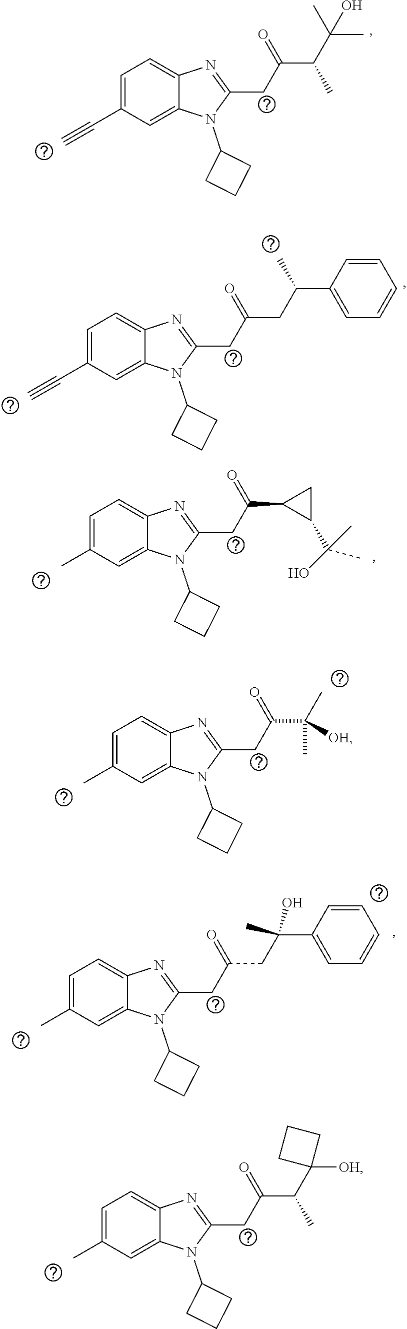

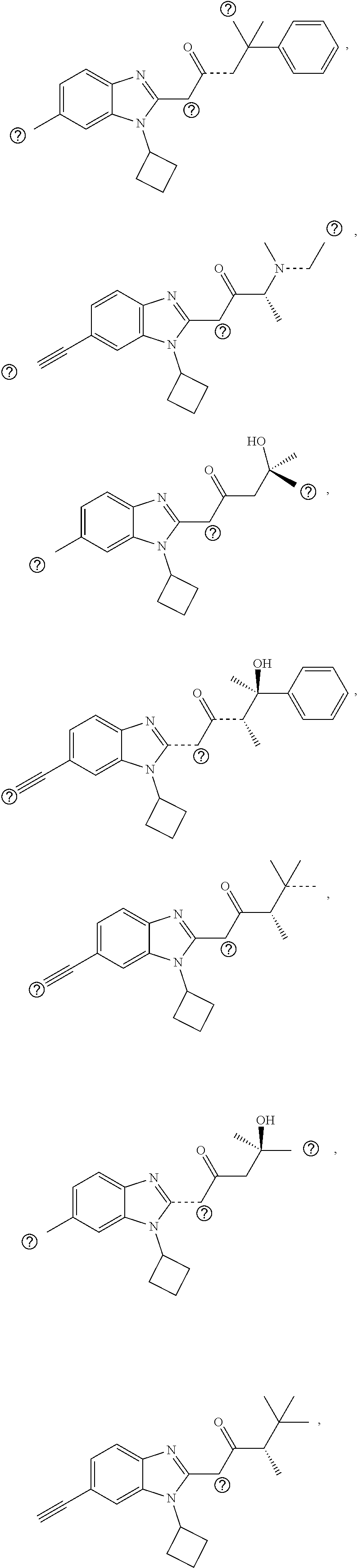

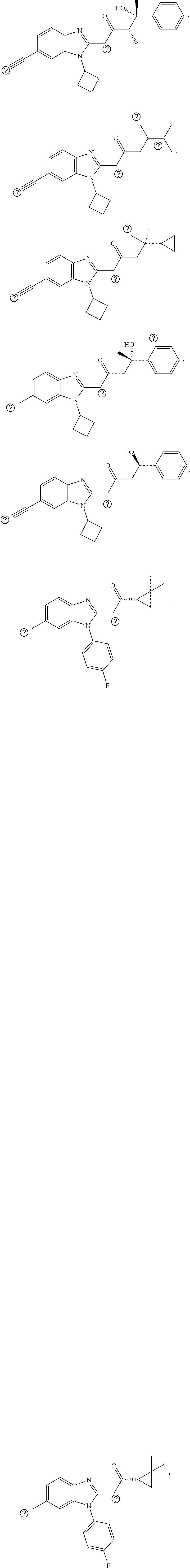

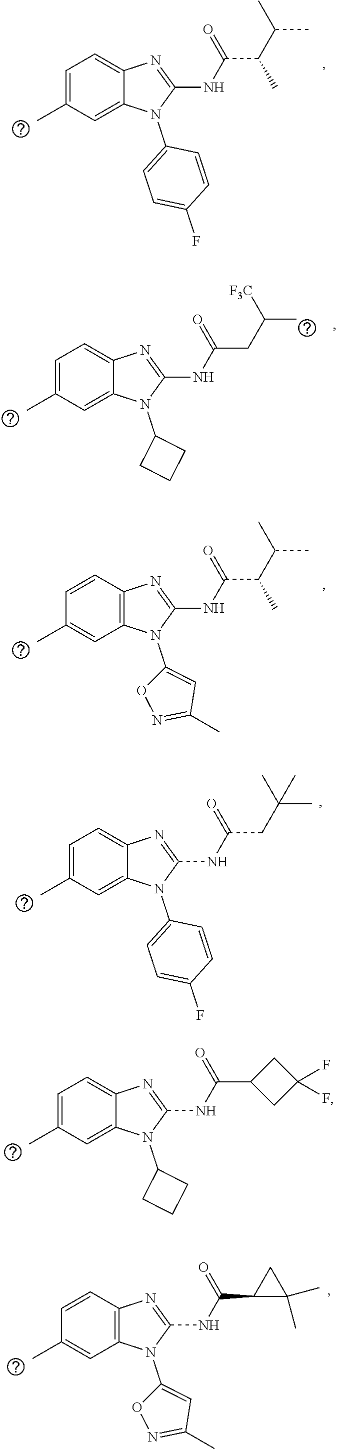

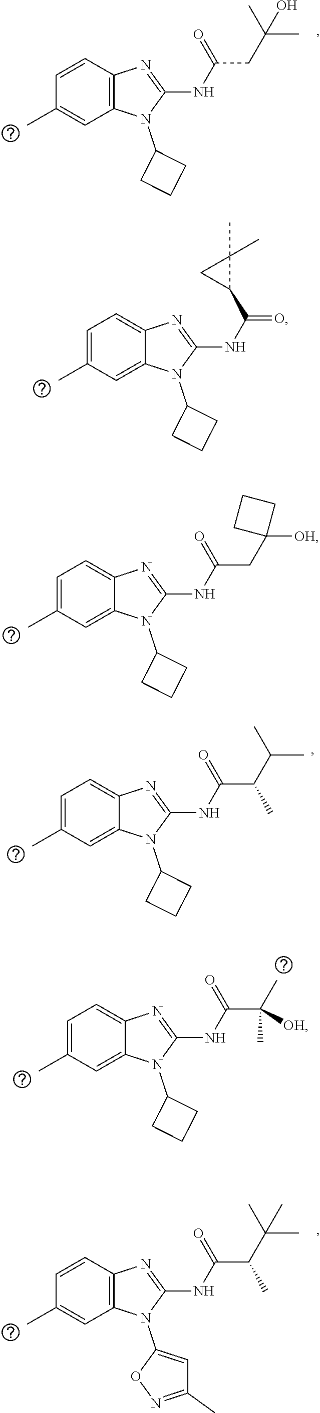

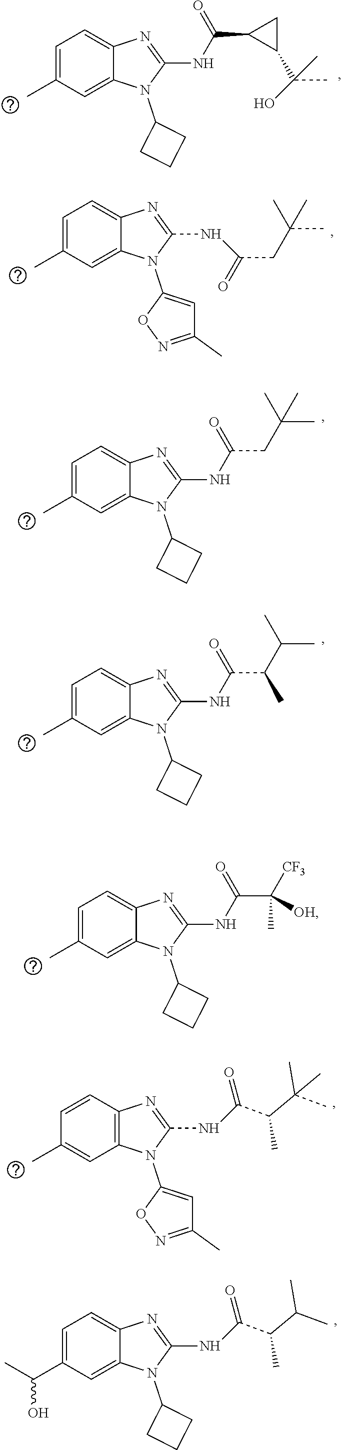

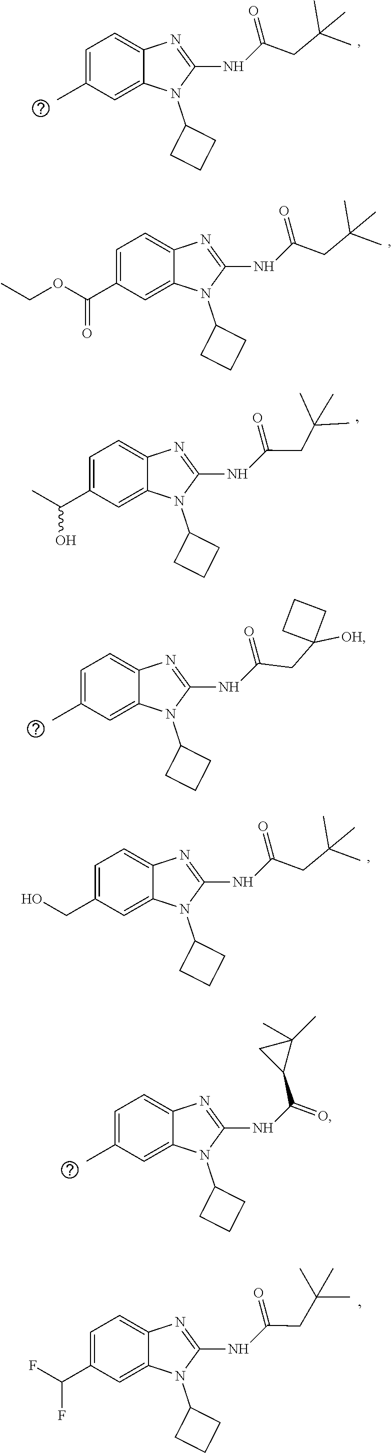

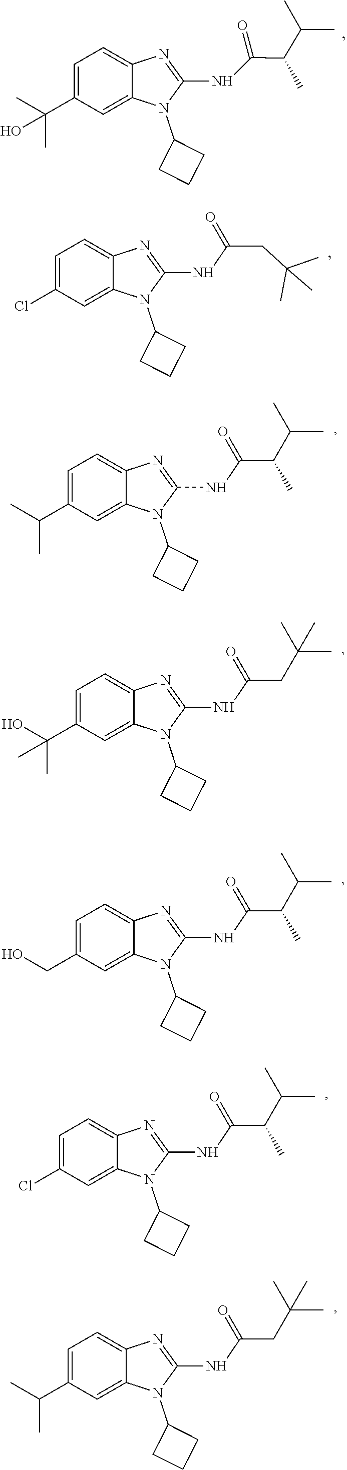

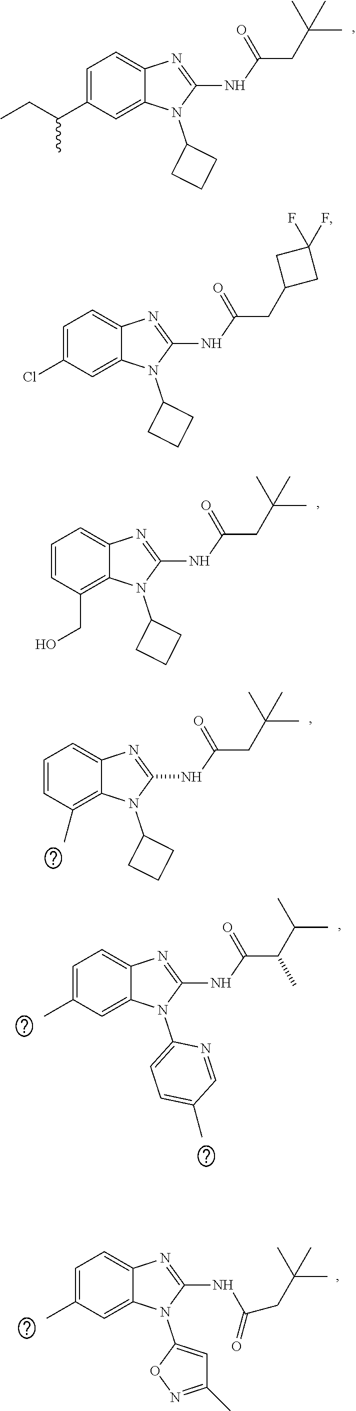

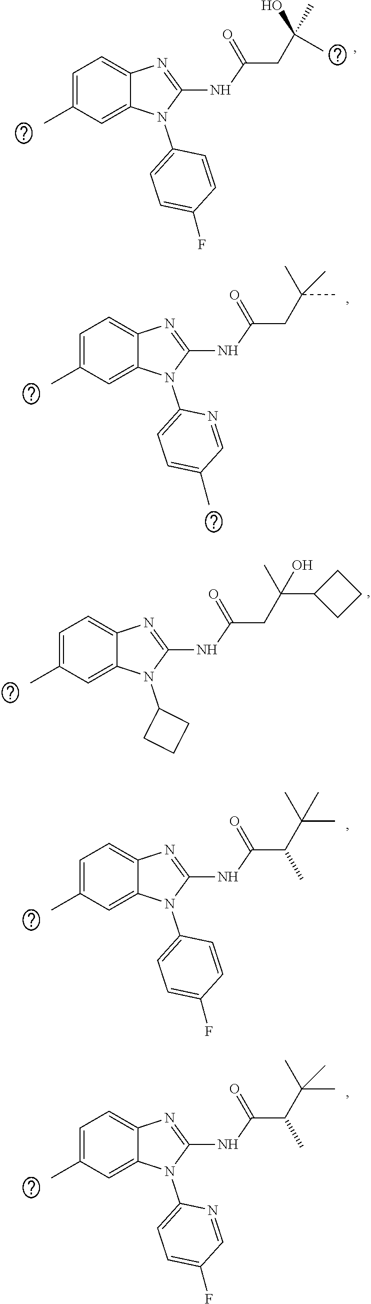

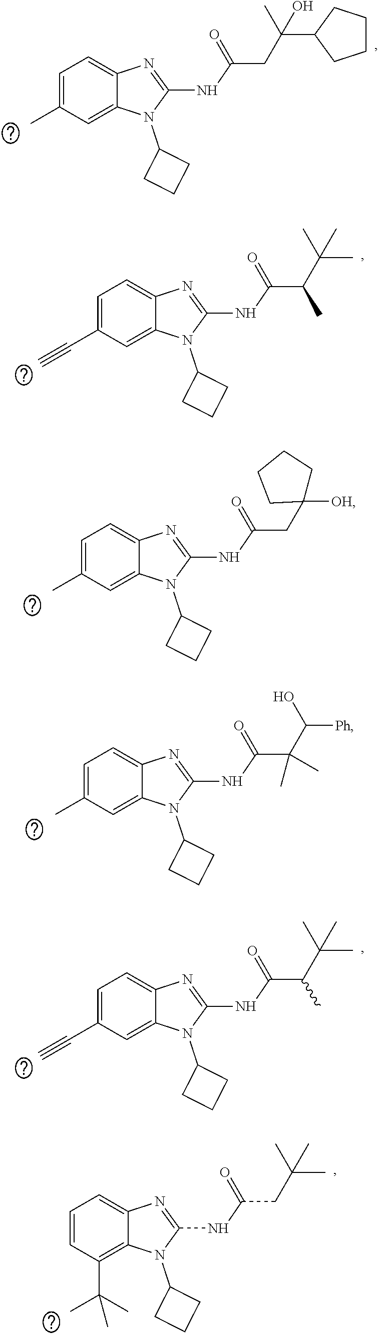

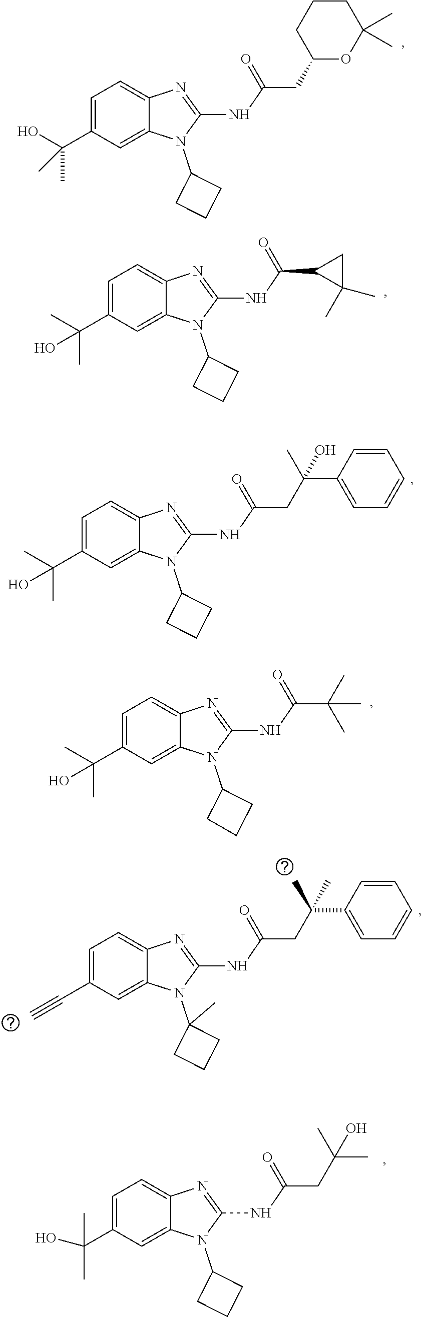

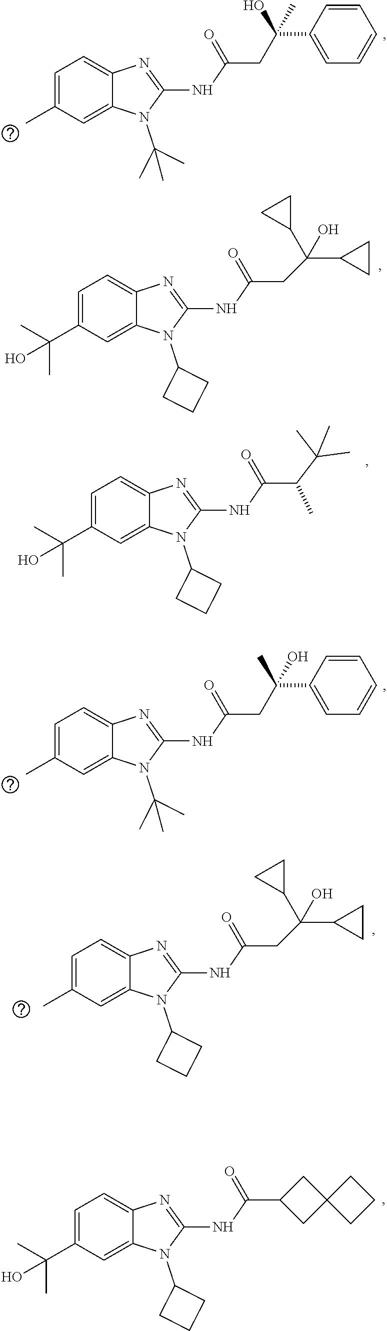

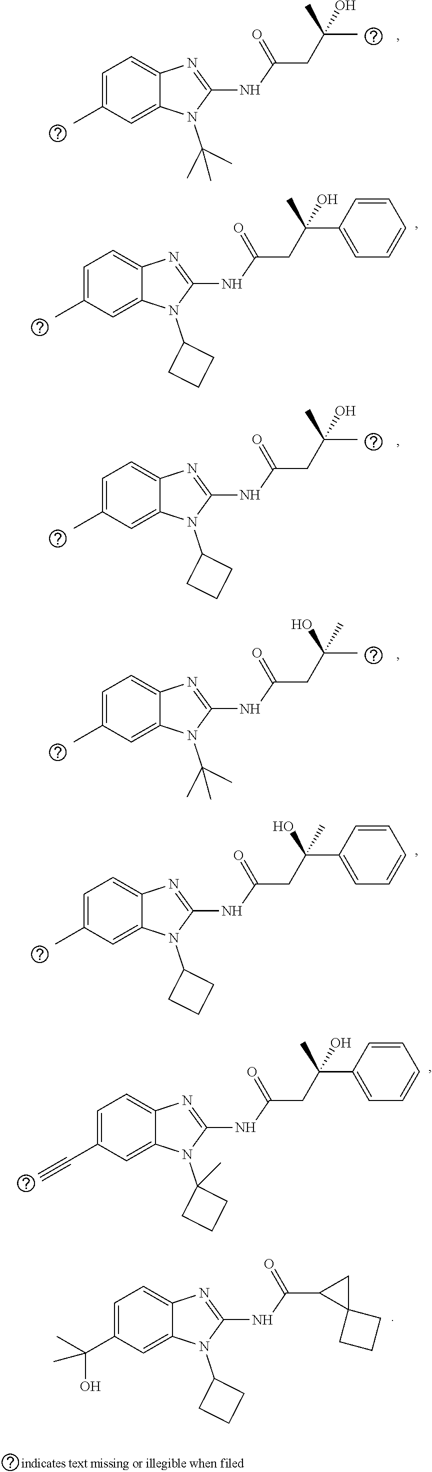

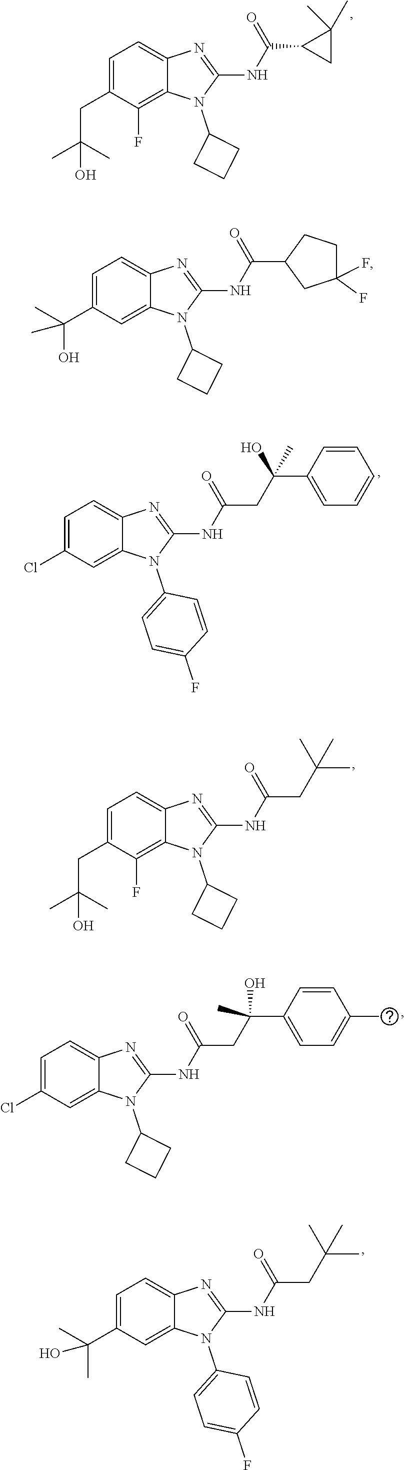

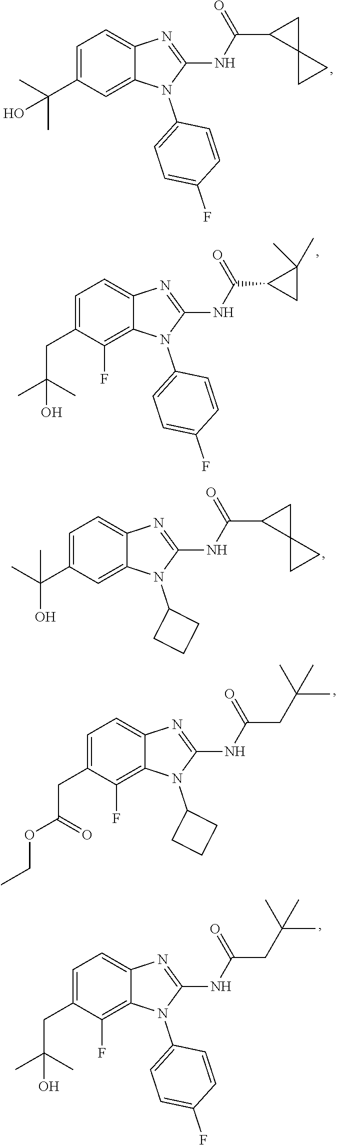

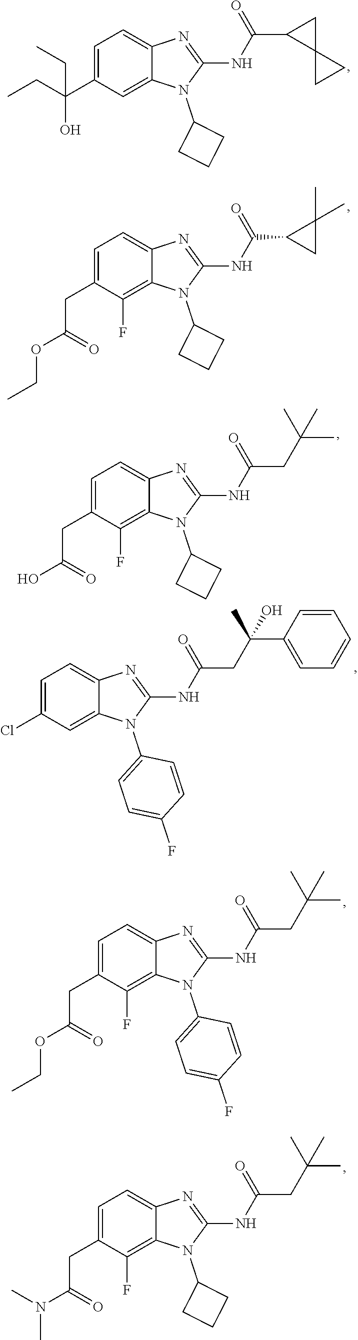

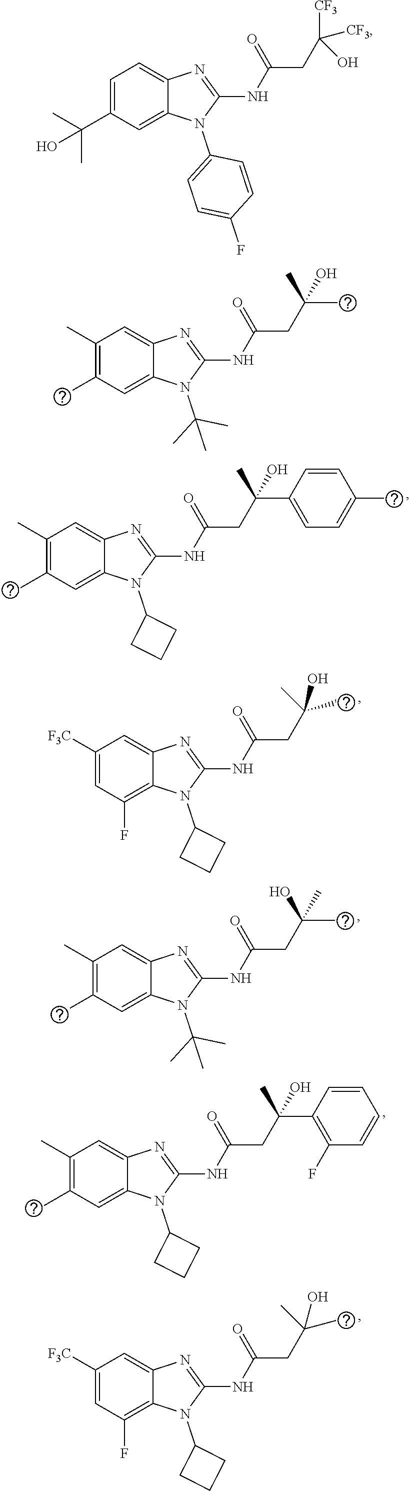

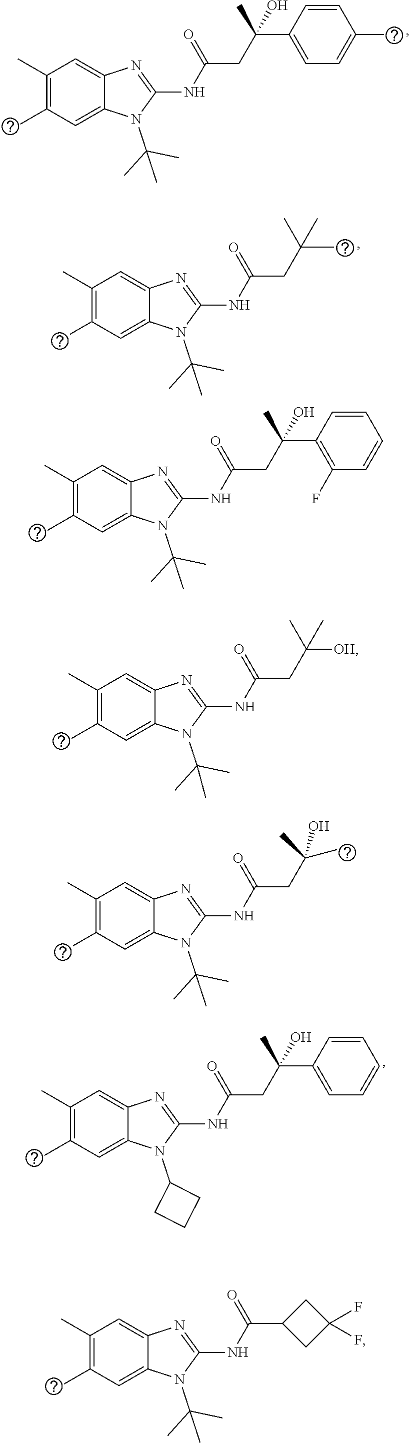

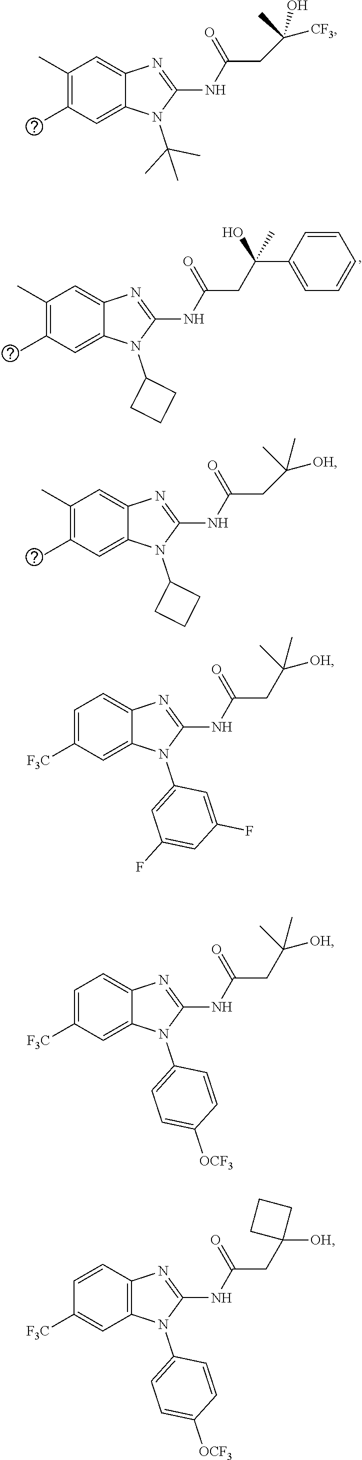 [SMILES: Cc1cc2nc(NC(=O)C[C@](C)(O)C(F)(F)F)n(C(C)(C)C)c2cc1C ; Cc1cc2nc(NC(=O)C[C@@](C)(O)c3ccccc3)n(C3CCC3)c2cc1C ; Cc1cc2nc(NC(=O)CC(C)(C)O)n(C3CCC3)c2cc1C ; CC(C)(O)CC(=O)Nc1nc2ccc(C(F)(F)F)cc2n1-c1cc(F)cc(F)c1 ; CC(C)(O)CC(=O)Nc1nc2ccc(C(F)(F)F)cc2n1-c1ccc(OC(F)(F)F)cc1 ; O=C(CC1(O)CCC1)Nc1nc2ccc(C(F)(F)F)cc2n1-c1ccc(OC(F)(F)F)cc1]

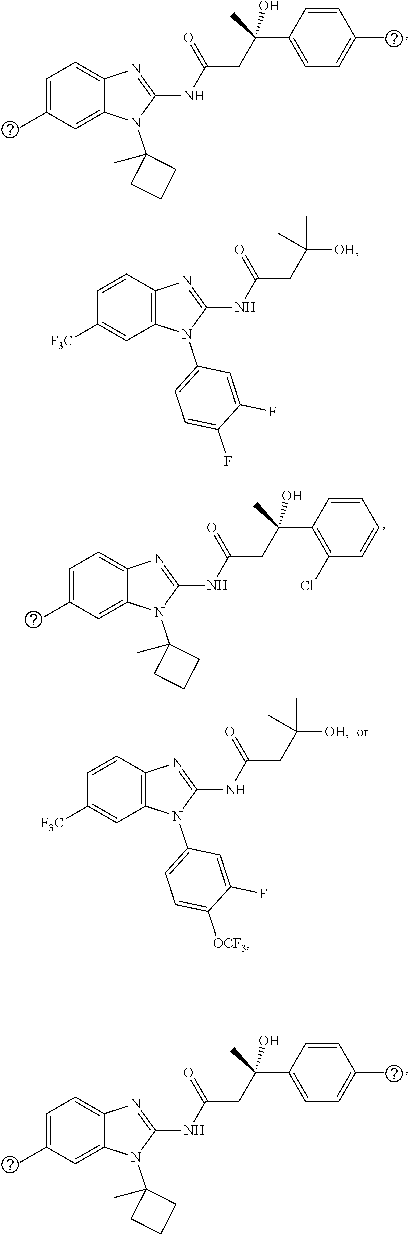

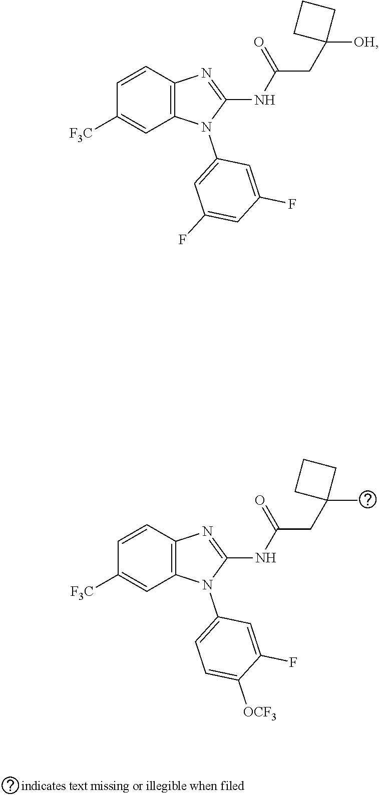 [SMILES: O=C(CC1(O)CCC1)Nc1nc2ccc(C(F)(F)F)cc2n1-c1cc(F)cc(F)c1 ; CC1(CC(=O)Nc2nc3ccc(C(F)(F)F)cc3n2-c2ccc(OC(F)(F)F)c(F)c2)CCC1]

In further embodiments, the Kv7 channel activator is a compound according to formula 2 wherein, D is optionally substituted cyclobutyl, or t-butyl; A is C₂₋₈ alkyl; X is H, CF₃, or optionally substituted phenyl; Y is H or OH; R¹ is CN or C₁₋₄ hydroxyalkyl; and R², R³, and R⁴ are independently H, or F.

In further embodiments, R¹ is CN, —C(CH₃)₂OH, or —CH₂C(CH₃)₂OH.

In further embodiments, R² is F.

In further embodiments, R³ is H.

In further embodiments, R⁴ is H.

In further embodiments, R¹ is CN.

In further embodiments, R¹ is C₁₋₄ hydroxyalkyl.

In further embodiments, X is optionally substituted phenyl.

In further embodiments, X is CF₃.

In further embodiments, X is H.

In further embodiments, Y is H.

In further embodiments, Y is OH.

In further embodiments, the Kv7 channel activator is a compound according to formula 2 wherein, D is cyclobutyl; A is C₁₋₈ alkyl; X is CF₃; Y is H; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃;

or
D is optionally substituted cyclobutyl; A is C₁₋₈ alkyl; X is CF₃; Y is H; R¹ is H, C₁, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃;
   or
D is t-butyl; A is C₁₋₈ alkyl; X is H; Y is H; R¹ is Cl, Br, CN, OCH₃, CF₃, —CO₂CH₂CH₃, C₁₋₄ alkyl, or C₁₋₄ hydroxyalkyl; R₂ is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃;
   or
D is t-butyl; A is C₁₋₈ alkyl; X is CF₃; Y is H; R¹ is H, Cl, Br, CN, OCH₃, CF₃, CO₂CH₂CH₃, C₁₋₄ alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃;
   or
D is cyclobutyl; A is C₁₋₈ alkyl; X is H; Y is methyl(2,2,2-trifluoroethyl)amino; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃;
   or
D is cyclobutyl; A is C₁₋₈ alkyl; X is CF₃; Y is dimethylamino; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃;
   or

D is optionally substituted cyclobutyl, optionally substituted phenyl, or optionally substituted C₂₋₅ alkyl, wherein the optional substituents are selected from —CH₃ and F; A is C₁ alkyl; X is substituted cyclobutyl, wherein the substituent is F; Y is H; R¹ is selected from H, C₃ hydroxyalkyl, CN, F, or Cl; R² is selected from H, CN, F, Br, or —OCF₃; R³ is selected from H, F, or —OCH₃; R⁴ is H or F; and wherein when X is substituted with 2 fluorine atoms, the fluorine atoms are not geminal; or a pharmaceutically acceptable salt thereof;

In an embodiment, the Kv7 channel activator is a compound according to formula 2. Wherein, R¹, R², R³, and R⁴ are independently H, F, Cl, Br, I, or a substituent having a molecular weight of 15 Da to 200 Da and consisting of 2 to 5 chemical elements, wherein the chemical elements are independently C, H, O, N, S, F, Cl, or Br.

In further embodiments, Y is H, F, CF₃, OH, C₁₋₅ O-alkyl, C₀₋₆ alkylamino, optionally substituted tetrahydropyranyl, or C₀₋₆ fluoroalkylamino.

In further embodiments, R¹ is H, Cl, Br, —OCH₃, —CN, —CF₃, —CH₂OH, —COOCH₂CH₃, —C(CH₃)₂OH, —CHOHCH₂CH₃, —CHOHCH₃, —CHF₂, —CH(CH₃)₂, —C(CH₂CH₃) OH, —CH₂COOCH₂CH₃, —CH₂C(CH₃)₂OH, —CH₂COOH, or —CH₂CON(CH₃)₂.

In further embodiments, R² is H, F, —CH₂OH, —CO₂Me, or —C(CH₃)₂OH.

In further embodiments, R³ is H.

In further embodiments, R⁴ is H, —CH₃, or —CF₃.

In further embodiments, the Kv7 channel activator is a compound selected from the

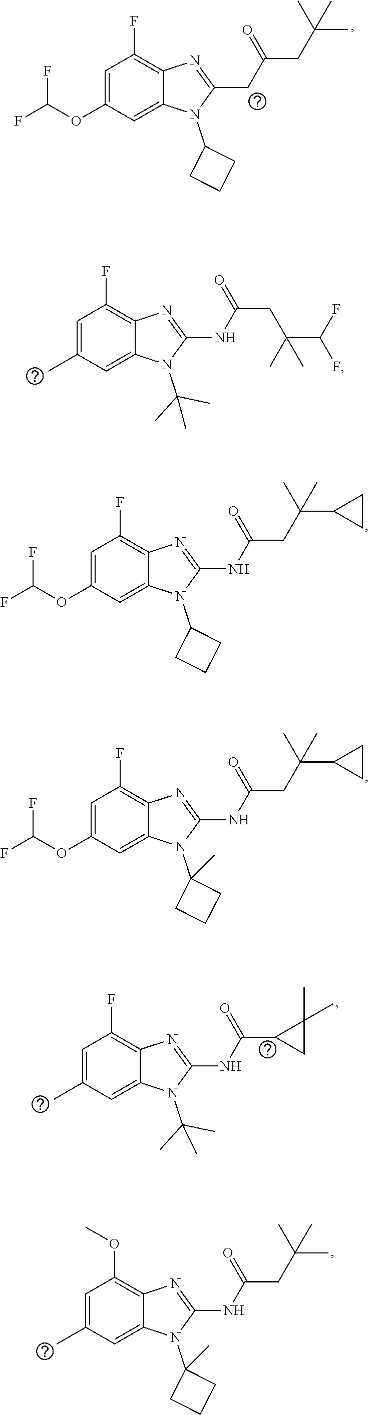

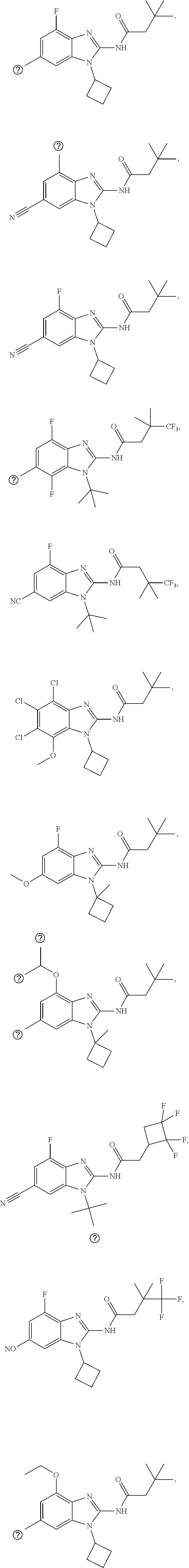

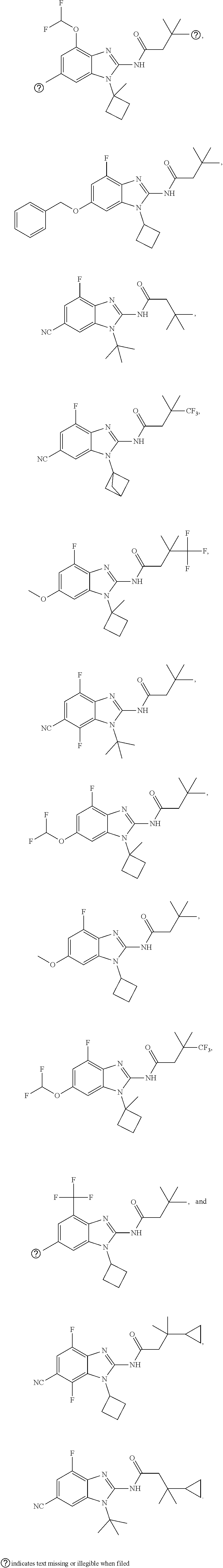

or a pharmaceutically acceptable salt thereof.

In further embodiments, the Kv7 channel activator is a compound selected from the

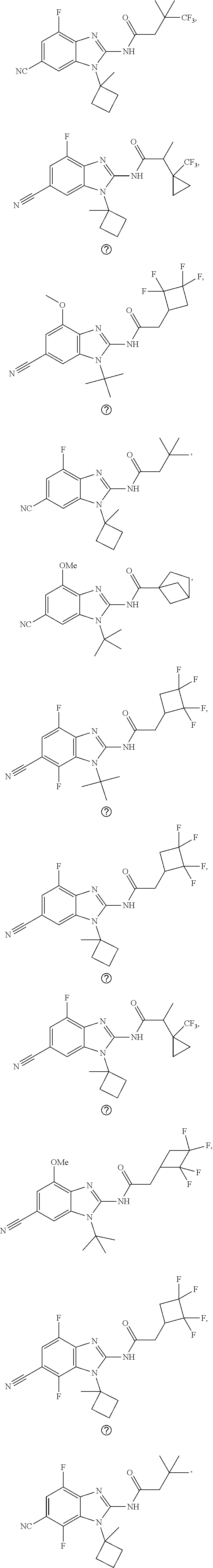

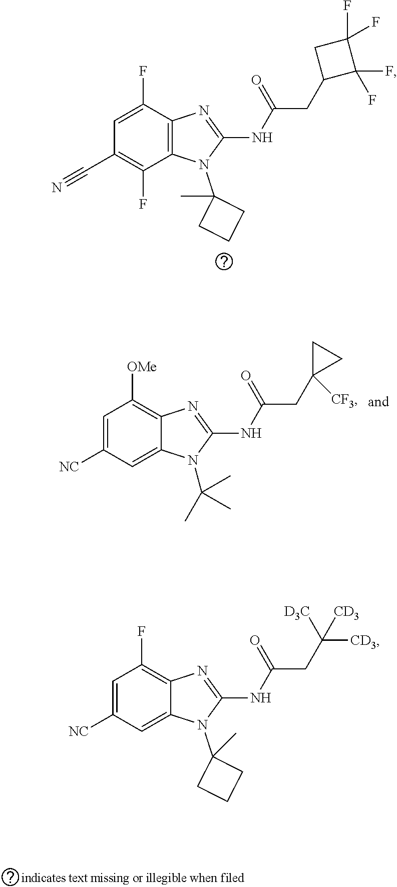 [SMILES: CC1(n2c(NC(=O)CC3CC(F)(F)C3(F)F)nc3c(F)cc(C#N)c(F)c32)CCC1 ; COc1cc(C#N)cc2c1nc(NC(=O)CC1(C(F)(F)F)CC1)n2C(C)(C)C ; [2H]C([2H])([2H])C(CC(=O)Nc1nc2c(F)cc(C#N)cc2n1C1(C)CCC1)(C([2H])([2H])[2H])C([2H])([2H])[2H]]

or a pharmaceutically acceptable salt thereof.

In further embodiments the Kv7 channel activator is a compound selected from the

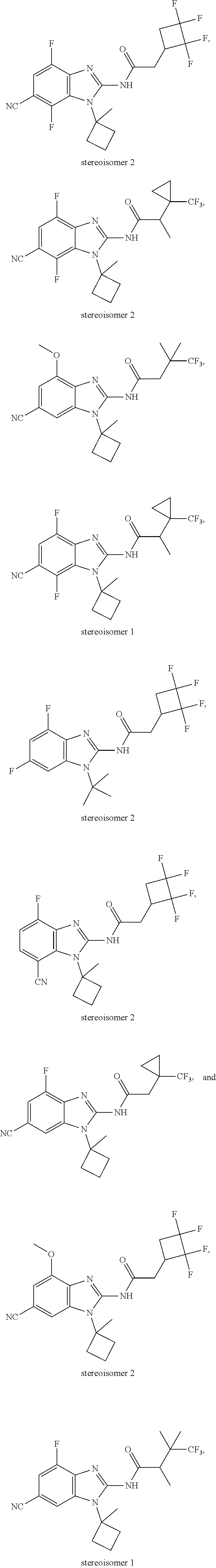

or a pharmaceutically acceptable salt thereof.

In further embodiments, the Kv7 channel activator is a compound selected from the

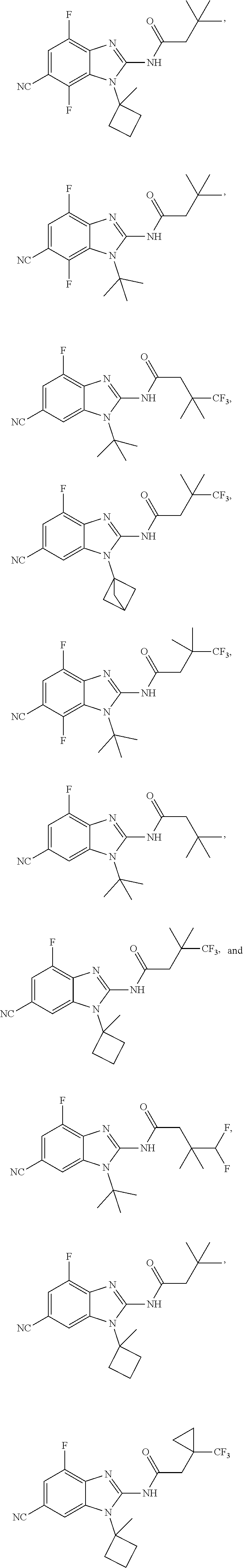

or a pharmaceutically acceptable salt thereof.

In further embodiments, the compound is:

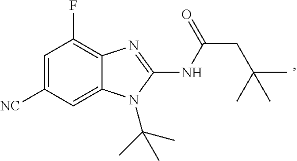 [SMILES: CC(C)(C)CC(=O)Nc1nc2c(F)cc(C#N)cc2n1C(C)(C)C]

or a pharmaceutically acceptable salt thereof.

In further embodiments, the compound is:

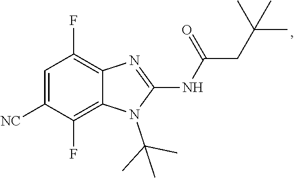 [SMILES: CC(C)(C)CC(=O)Nc1nc2c(F)cc(C#N)c(F)c2n1C(C)(C)C]

or a pharmaceutically acceptable salt thereof.

In further embodiments, the compound is:

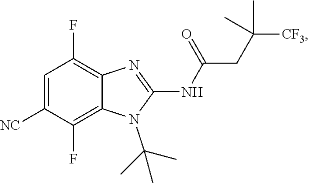 [SMILES: CC(C)(C)n1c(NC(=O)CC(C)(C)C(F)(F)F)nc2c(F)cc(C#N)c(F)c21]

or a pharmaceutically acceptable salt thereof.

In further embodiments, the compound is:

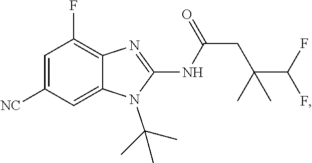 [SMILES: CC(C)(CC(=O)Nc1nc2c(F)cc(C#N)cc2n1C(C)(C)C)C(F)F]

or a pharmaceutically acceptable salt thereof.

In further embodiments, the compound is:

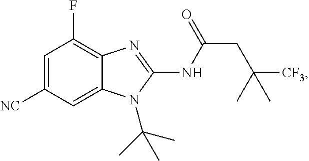 [SMILES: CC(C)(C)n1c(NC(=O)CC(C)(C)C(F)(F)F)nc2c(F)cc(C#N)cc21]

or a pharmaceutically acceptable salt thereof.

In further embodiments, the compound is:

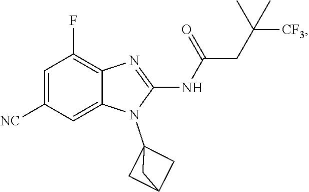 [SMILES: CC(C)(CC(=O)Nc1nc2c(F)cc(C#N)cc2n1C12CC(C1)C2)C(F)(F)F]

or a pharmaceutically acceptable salt thereof.

In further embodiments, the compound is:

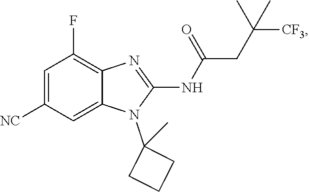 [SMILES: CC1(n2c(NC(=O)CC(C)(C)C(F)(F)F)nc3c(F)cc(C#N)cc32)CCC1]

or a pharmaceutically acceptable salt thereof.

In further embodiments, the compound is:

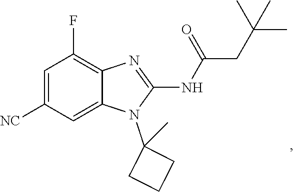 [SMILES: CC(C)(C)CC(=O)Nc1nc2c(F)cc(C#N)cc2n1C1(C)CCC1]

or a pharmaceutically acceptable salt thereof.

In further embodiments, the compound is:

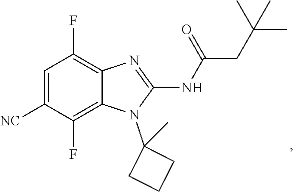 [SMILES: CC(C)(C)CC(=O)Nc1nc2c(F)cc(C#N)c(F)c2n1C1(C)CCC1]

or a pharmaceutically acceptable salt thereof.

In further embodiments, the compound is:

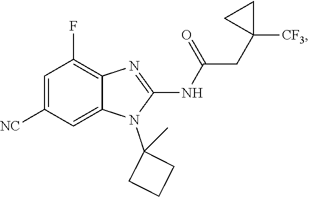 [SMILES: CC1(n2c(NC(=O)CC3(C(F)(F)F)CC3)nc3c(F)cc(C#N)cc32)CCC1]

or a pharmaceutically acceptable salt thereof.

In further embodiments, the Kv7 channel activator is a compound selected from the

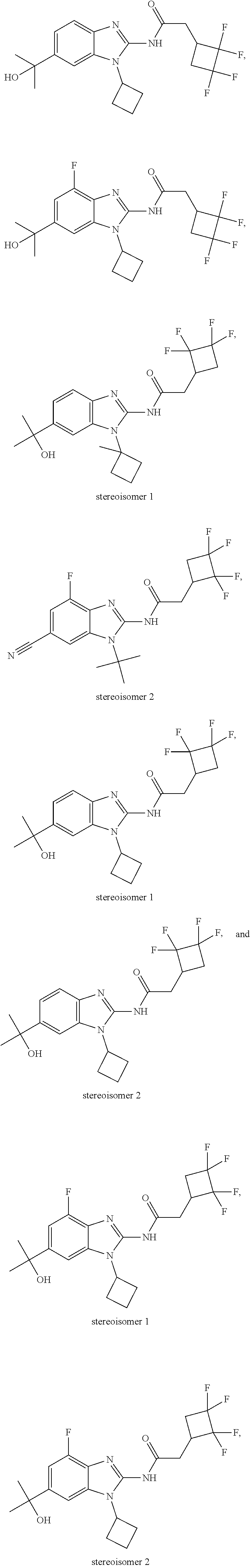

or a pharmaceutically acceptable salt thereof.

In further embodiments, the Kv7 channel activator is a compound selected from the

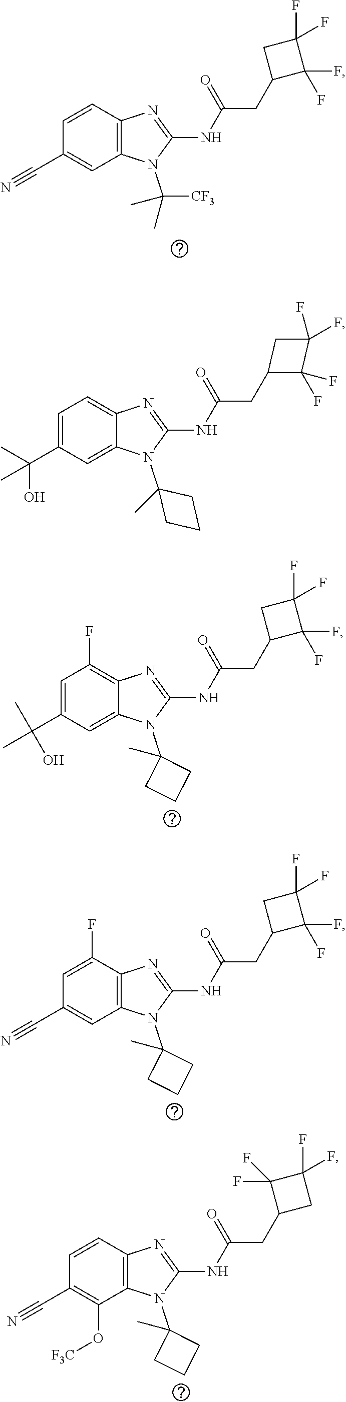 [SMILES: CC(C)(n1c(NC(=O)CC2CC(F)(F)C2(F)F)nc2ccc(C#N)cc21)C(F)(F)F ; CC(C)(O)c1ccc2nc(NC(=O)CC3CC(F)(F)C3(F)F)n(C3(C)CCC3)c2c1 ; CC(C)(O)c1cc(F)c2nc(NC(=O)CC3CC(F)(F)C3(F)F)n(C3(C)CCC3)c2c1 ; CC1(n2c(NC(=O)CC3CC(F)(F)C3(F)F)nc3c(F)cc(C#N)cc32)CCC1 ; CC1(n2c(NC(=O)CC3CC(F)(F)C3(F)F)nc3ccc(C#N)c(OC(F)(F)F)c32)CCC1]

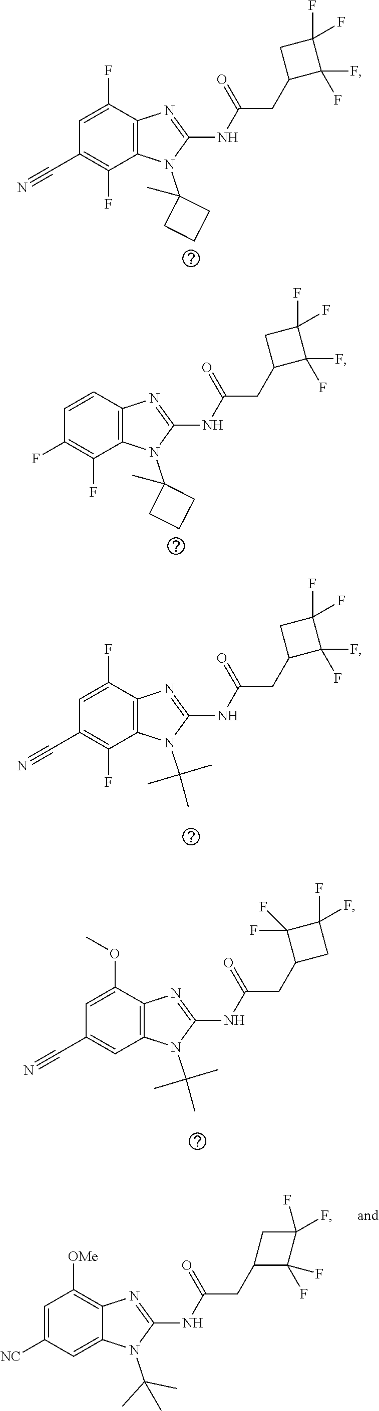 [SMILES: CC1(n2c(NC(=O)CC3CC(F)(F)C3(F)F)nc3c(F)cc(C#N)c(F)c32)CCC1 ; CC1(n2c(NC(=O)CC3CC(F)(F)C3(F)F)nc3ccc(F)c(F)c32)CCC1 ; CC(C)(C)n1c(NC(=O)CC2CC(F)(F)C2(F)F)nc2c(F)cc(C#N)c(F)c21 ; COc1cc(C#N)cc2c1nc(NC(=O)CC1CC(F)(F)C1(F)F)n2C(C)(C)C ; COc1cc(C#N)cc2c1nc(NC(=O)CC1CC(F)(F)C1(F)F)n2C(C)(C)C ; "and"]

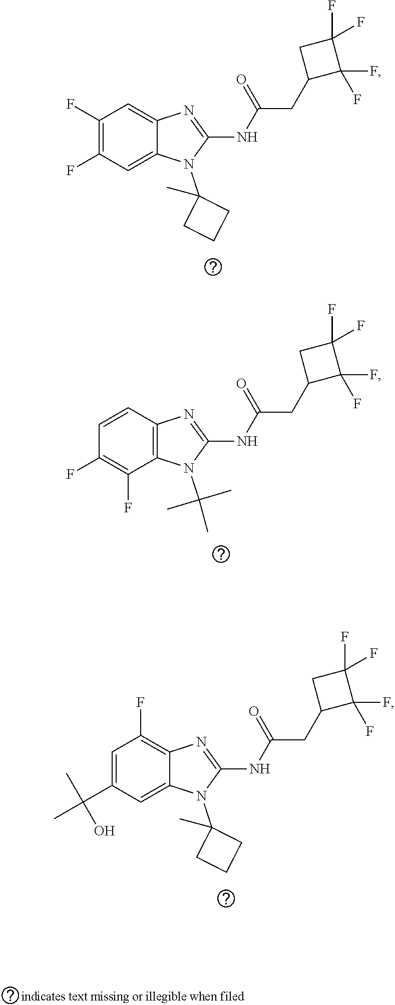 [SMILES: CC1(n2c(NC(=O)CC3CC(F)(F)C3(F)F)nc3cc(F)c(F)cc32)CCC1 ; CC(C)(C)n1c(NC(=O)CC2CC(F)(F)C2(F)F)nc2ccc(F)c(F)c21 ; CC(C)(O)c1cc(F)c2nc(NC(=O)CC3CC(F)(F)C3(F)F)n(C3(C)CCC3)c2c1]

or a pharmaceutically acceptable salt thereof.

In further embodiments, the Kv7 channel activator is a compound selected from the

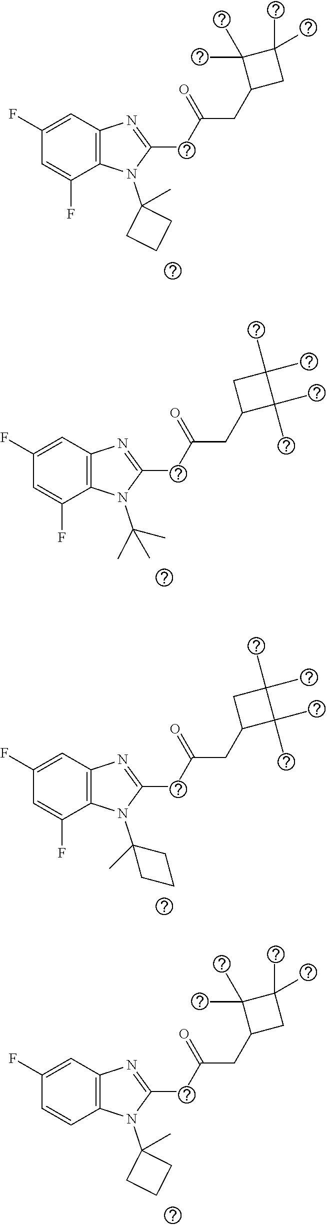 [SMILES: CC1(n2c(CC(=O)CC3CC(C)(C)C3(C)C)nc3cc(F)cc(F)c32)CCC1 ; CC(C)(C)n1c(CC(=O)CC2CC(C)(C)C2(C)C)nc2cc(F)cc(F)c21 ; CC1(n2c(CC(=O)CC3CC(C)(C)C3(C)C)nc3cc(F)cc(F)c32)CCC1 ; CC1(n2c(CC(=O)CC3CC(C)(C)C3(C)C)nc3cc(F)ccc32)CCC1]

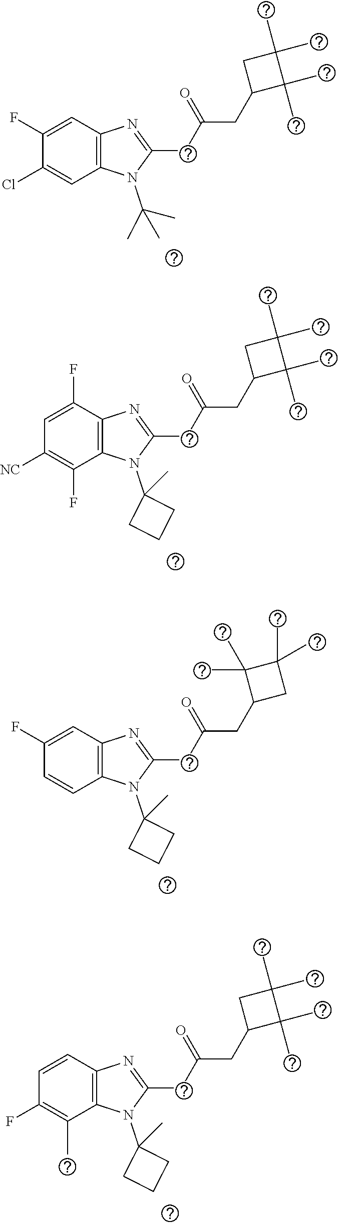 [SMILES: CC(C)(C)n1c(CC(=O)CC2CC(C)(C)C2(C)C)nc2cc(F)c(Cl)cc21 ; CC1(n2c(CC(=O)CC3CC(C)(C)C3(C)C)nc3c(F)cc(C#N)c(F)c32)CCC1 ; CC1(n2c(CC(=O)CC3CC(C)(C)C3(C)C)nc3cc(F)ccc32)CCC1 ; Cc1c(F)ccc2nc(CC(=O)CC3CC(C)(C)C3(C)C)n(C3(C)CCC3)c12]

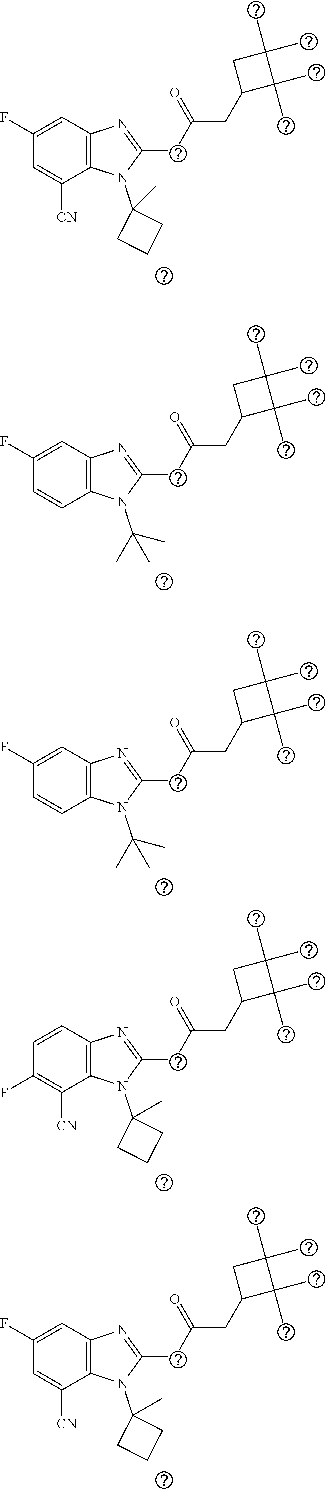 [SMILES: CC1(n2c(CC(=O)CC3CC(C)(C)C3(C)C)nc3cc(F)cc(C#N)c32)CCC1 ; CC(C)(C)n1c(CC(=O)CC2CC(C)(C)C2(C)C)nc2cc(F)ccc21 ; CC(C)(C)n1c(CC(=O)CC2CC(C)(C)C2(C)C)nc2cc(F)ccc21 ; CC1(n2c(CC(=O)CC3CC(C)(C)C3(C)C)nc3ccc(F)c(C#N)c32)CCC1 ; CC1(n2c(CC(=O)CC3CC(C)(C)C3(C)C)nc3cc(F)cc(C#N)c32)CCC1]

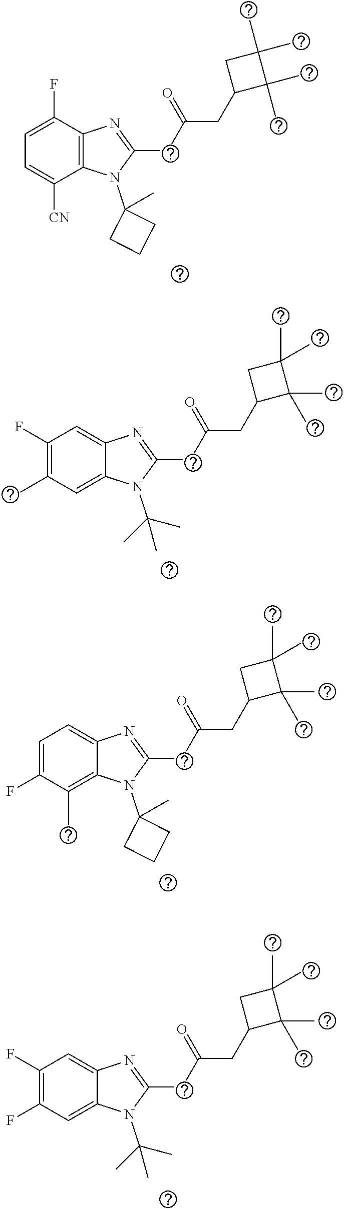 [SMILES: CC1(n2c(CC(=O)CC3CC(C)(C)C3(C)C)nc3c(F)ccc(C#N)c32)CCC1 ; Cc1cc2c(cc1F)nc(CC(=O)CC1CC(C)(C)C1(C)C)n2C(C)(C)C ; Cc1c(F)ccc2nc(CC(=O)CC3CC(C)(C)C3(C)C)n(C3(C)CCC3)c12 ; CC(C)(C)n1c(CC(=O)CC2CC(C)(C)C2(C)C)nc2cc(F)c(F)cc21]

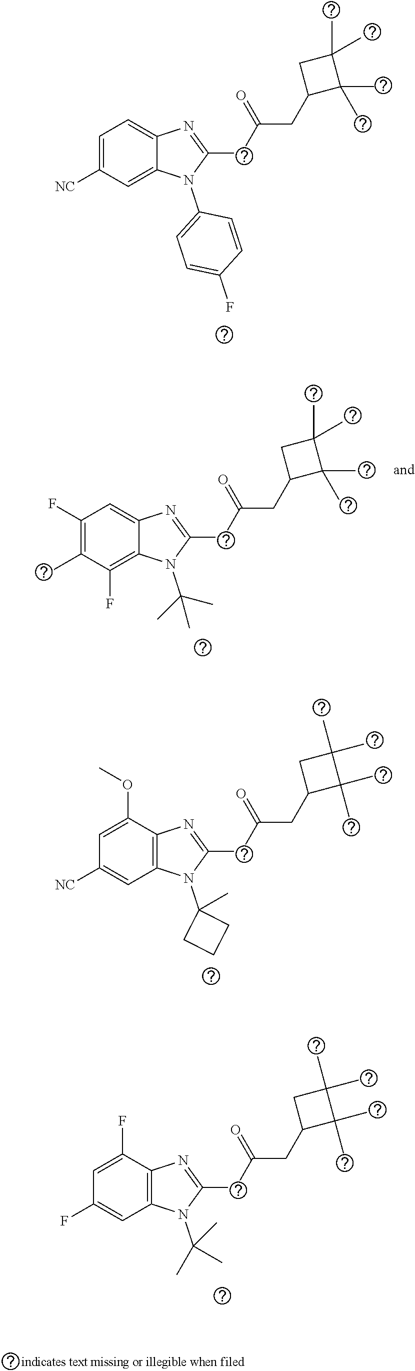 [SMILES: CC1(C)CC(CC(=O)Cc2nc3ccc(C#N)cc3n2-c2ccc(F)cc2)C1(C)C ; Cc1c(F)cc2nc(CC(=O)CC3CC(C)(C)C3(C)C)n(C(C)(C)C)c2c1F ; COc1cc(C#N)cc2c1nc(CC(=O)CC1CC(C)(C)C1(C)C)n2C1(C)CCC1 ; CC(C)(C)n1c(CC(=O)CC2CC(C)(C)C2(C)C)nc2c(F)cc(F)cc21]

In further embodiments, the compound is:

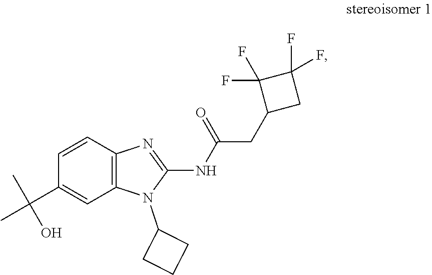 [SMILES: CC(C)(O)c1ccc2nc(NC(=O)CC3CC(F)(F)C3(F)F)n(C3CCC3)c2c1]

or a pharmaceutically acceptable salt thereof.

In further embodiments, the compound is:

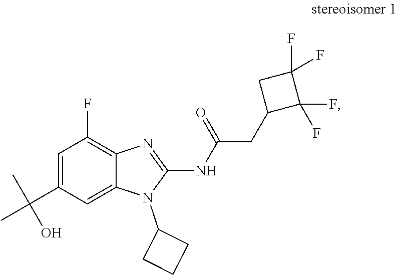 [SMILES: CC(C)(O)c1cc(F)c2nc(NC(=O)CC3CC(F)(F)C3(F)F)n(C3CCC3)c2c1]

or a pharmaceutically acceptable salt thereof.

In further embodiments, the compound is:

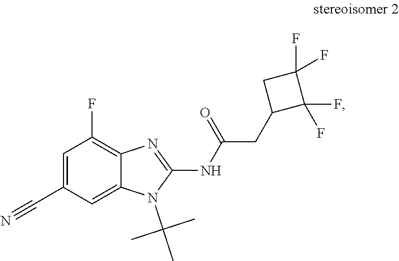 [SMILES: CC(C)(C)n1c(NC(=O)CC2CC(F)(F)C2(F)F)nc2c(F)cc(C#N)cc21]

or a pharmaceutically acceptable salt thereof.

In further embodiments, the compound is:

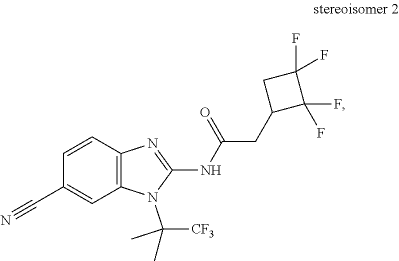 [SMILES: CC(C)(n1c(NC(=O)CC2CC(F)(F)C2(F)F)nc2ccc(C#N)cc21)C(F)(F)F]

or a pharmaceutically acceptable salt thereof.

In further embodiments, the compound is:

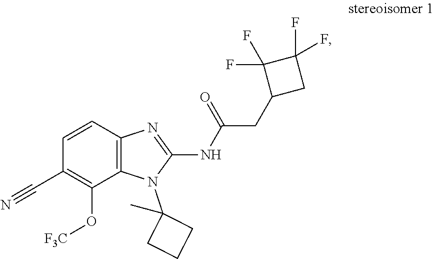 [SMILES: CC1(n2c(NC(=O)CC3CC(F)(F)C3(F)F)nc3ccc(C#N)c(OC(F)(F)F)c32)CCC1]

or a pharmaceutically acceptable salt thereof.

In further embodiments, the compound is:

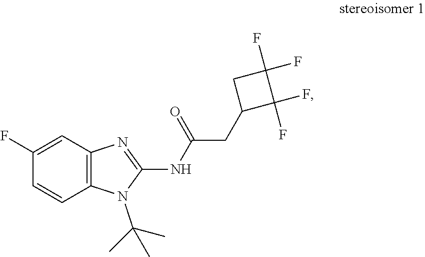 [SMILES: CC(C)(C)n1c(NC(=O)CC2CC(F)(F)C2(F)F)nc2cc(F)ccc21]

or a pharmaceutically acceptable salt thereof.

In further embodiments, the compound is:

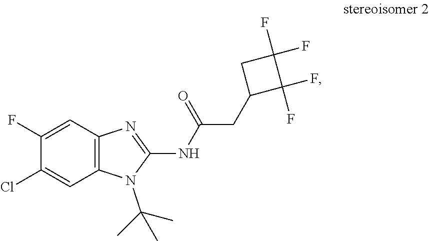 [SMILES: CC(C)(C)n1c(NC(=O)CC2CC(F)(F)C2(F)F)nc2cc(F)c(Cl)cc21]

or a pharmaceutically acceptable salt thereof.

In further embodiments, the compound is:

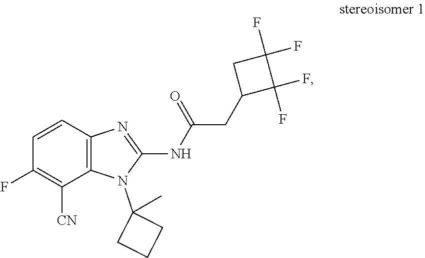 [SMILES: CC1(n2c(NC(=O)CC3CC(F)(F)C3(F)F)nc3ccc(F)c(C#N)c32)CCC1]

or a pharmaceutically acceptable salt thereof.

In further embodiments, the compound is:

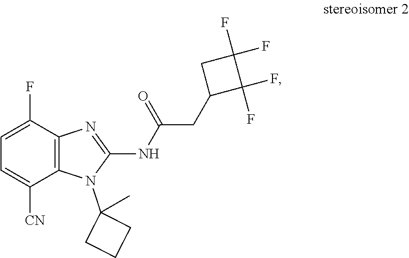 [SMILES: CC1(n2c(NC(=O)CC3CC(F)(F)C3(F)F)nc3c(F)ccc(C#N)c32)CCC1]

or a pharmaceutically acceptable salt thereof.

In further embodiments, the compound is:

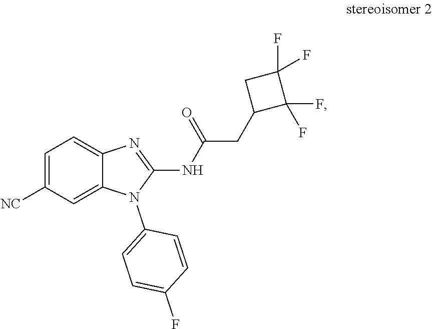 [SMILES: N#Cc1ccc2nc(NC(=O)CC3CC(F)(F)C3(F)F)n(-c3ccc(F)cc3)c2c1]

or a pharmaceutically acceptable salt thereof.

In further embodiments, the compound is:

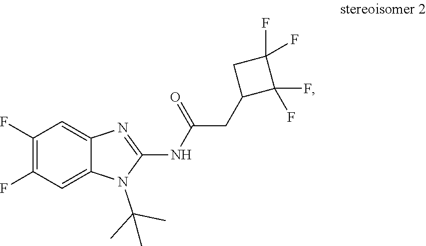 [SMILES: CC(C)(C)n1c(NC(=O)CC2CC(F)(F)C2(F)F)nc2cc(F)c(F)cc21]

or a pharmaceutically acceptable salt thereof.

In further embodiments, the compound is:

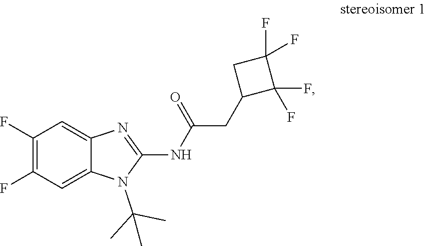 [SMILES: CC(C)(C)n1c(NC(=O)CC2CC(F)(F)C2(F)F)nc2cc(F)c(F)cc21]

or a pharmaceutically acceptable salt thereof.

In an embodiment of Formula 2, D is optionally substituted cyclobutyl, or t-butyl; A is C₂₋₈ alkyl, X is H, CF₃, or optionally substituted phenyl, Y is H or OH, R¹ is CN or C₁₋₄ hydroxyalkyl; and R², R³, and R⁴ are independently H, or F. In a further embodiment, R¹ is CN, —C(CH₃)₂OH, or —CH₂C(CH₃)₂OH. In a further embodiment, R² is F. In a further embodiment, R³ is H. In a further embodiment, R⁴ is H. In a further embodiment, R¹ is CN. In a further embodiment, R¹ is C₁₋₄ hydroxyalkyl. In a further embodiment, X is optionally substituted phenyl. In a further embodiment, X is CF₃. In a further embodiment, X is H. In a further embodiment, Y is H. In a further embodiment, Y is OH.

In an embodiment of Formula 2, D is cyclobutyl; A is C₁₋₈ alkyl, X is CF₃, Y is H, R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl or B; and R⁴ is H, —CH₃, or —CF₃.

In an embodiment of Formula 2, D is optionally substituted cyclobutyl, A is C₁₋₈ alkyl, X is CH₃, Y is H, R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —C(CH₃)₂OH; R³ is H; and R⁴ is H, —CH₃, or —CF₃.

In an embodiment of Formula 2, D is t-butyl; A is C₁₋₈ alkyl; X is H; Y is H; R¹ is C₁, Br, CN, OCH₃, CF₃, —CO₂CH₂CH₃, C₁₋₄alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, —CH₃, or —CF₃.

In an embodiment of Formula 2, D is t-butyl; A is C₁₋₈ alkyl; X is CF₃; Y is H; R¹ is H, Cl, Br, CN, OCH₃, CF₃, —CO₂CH₂CH₃, C₁₋₄alkyl, or C₁₋₄ hydroxyalkyl; R² is H, F, —CH₂OH, —CO₂Me, or —C(CH₃)₂OH; R³ is H, F, Cl, Br, I, or a substituent having a molecular weight of 15 Da to 200 Da and consisting of 2 to 5 chemical elements, wherein the chemical elements are independently C, H, O, N, S, F, Cl, or Br; and R⁴ is H, CH₃, or CF₃.

In an embodiment of Formula 2, D is cyclobutyl; A is C₁₋₈ alkyl; X is H; Y is methyl(2,2,2-trifluoroethyl)amino; R¹ is H, Cl, Br, CN, OCH₃, CF₃, —CO₂CH₂CH₃, C₁₋₄ alkyl, or C₁₋₄ hydroxyalkyl; R² is H, F, —CH₂OH, —CO₂Me, or —CH(CH₃)₂OH; R³ is H; and R⁴ is H, —CH₃, or —CF₃.

In an embodiment of Formula 2, D is optionally substituted cyclobutyl, optionally substituted phenyl, or optionally substituted C₂₋₅ alkyl, wherein the optional substituents are selected from —CH₃ and F; A is C₁ alkyl, X is substituted cyclobutyl, wherein the substituent is F; Y is H; R¹ is selected from H, C₃ hydroxyalkyl, CN, F, or Cl; R² is selected from H, CN, F, Br, or —OCF₃; R³ is selected from H, F, or —OCH₃; R⁴ is H or F; and wherein when X is substituted with 2 fluorine atoms, the fluorine atoms are not geminal; or a pharmaceutically acceptable salt thereof.

### Formula 3

In an embodiment, the Kv7 channel activator may be selected from one of the following compounds according to Formula 3. Such compounds are described in U.S. Pat. No. 8,293,911 issued on Oct. 23, 2012 and corresponding to U.S. application Ser. No. 11/894,877 filed Aug. 22, 2007; which is incorporated by reference in its entirety herein. In the case of any conflict of terminology in the context of Formula 3, this reference incorporated by reference herein controls.

In an embodiment, the Kv7 channel activator is a compound according to formula 3:

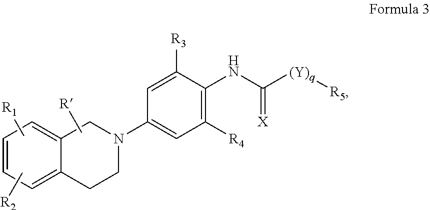

wherein, R₁ and R₂, vary independently, and are selected from the group consisting of H, CN, halogen, CH₂CN, OH, CH₂F, CHF₂, CF₃, CF₂CF₃, C₁-C₆ alkyl, C(═O)C₁-C₆ alkyl; NH—C₁-C₆ alkyl; N(C₁-C₆ alkyl)-C₁-C₆ alkyl, NHC(═O)C₁-C₆ alkyl, C(═O)N(CH₃)₂, C(═O)N(Et)₂, C(═O)NH₂, C(═O)NH—C₁-C₆alkyl, SO₂NH₂, NHSO₂-C₁-C₆ alkyl; C(═O)OC₁-C₆ alkyl, OC(═O)C₁-C₆ alkyl, OC₁-C₆ alkyl, SC₁-C₆ alkyl, C₃-C₆ cycloalkyl, (CH₂)ₘC₃-C₆ cycloalkyl, C₃-C₆ cycloalkenyl, (CH₂)ₘC₃-C₆ cycloalkenyl, C₂-C₆ alkenyl, C₂-C₆ alkynyl, Ar, (CH₂)ₘthienyl, (CH₂)ₘimidazolyl, (CH₂)ₘpyrazyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, and (CH₂)ₘpyrimidyl, Wherein m=zero, 1, or 2; Ar is a 5- to 10-member mono- or bicyclic aromatic group, optionally containing 1-4 ring heteroatoms selected independently from N, O, and S; or R₁ and R₂, together with the ring carbon atoms to which they are attached, form a 5- or 6-member fused ring; wherein said fused ring may be saturated, unsaturated, or aromatic, and said fused ring optionally contains one or two heteroatoms selected independently from the group consisting of O, N, and S; R′ is selected from the group consisting of H, halogen, phenyl, 2-(N,N-dimethylamino)ethyl, CF₃, OC₁-C₃ alkyl and C₁-C₃ alkyl; R₃ and R₄ vary independently, and are selected from the group consisting of H, CN, halogen, CF₃, OCF₃, OC₁-C₃ alkyl, and C₁-C₃ alkyl; X is O or S; Y is O or S; q=1 or zero; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar, wherein w=zero, 1, 2, or 3; Ar is a 5- to 10-member mono- or bicyclic aromatic group, optionally containing 1-4 ring heteroatoms selected independently from the group consisting of N, O, and S; and R₆ is selected from the group consisting of H or C₁-C₃ alkyl; where all cycloalkyl and cycloalkenyl optionally contain one or two ring heteroatoms selected independently from N, O, and S; wherein all alkyl, cycloalkyl, alkenyl, cycloalkenyl, heterocycloalkyl, heterocycloalkenyl, alkynyl, aryl, and heteroaryl groups in R₁, R₂, R′, R₃, R₄, R₅, R₆, and Ar are optionally substituted with one or two substituents selected independently from the group consisting of C₁-C₃ alkyl, halogen, OH, OEt, OMe, CN, CH₂F, OCF₃, and CF₃; and wherein, additionally, all cycloalkyl and heterocycloalkyl groups are optionally substituted with a carbonyl group.

In further embodiments, R₁ and R₂, vary independently, and are selected from the group consisting of H, halogen, CF₃, C₁-C₆ alkyl, C(═O)C₁-C₆ alkyl, C(═O)OC₁-C₆ alkyl, OC(═O)C₁-C₆ alkyl, OC₁-C₆ alkyl, SCH₃, C₃-C₆ cycloalkyl, (CH₂)ₘC₃-C₆ cycloalkyl, phenyl, pyridyl, pyrrolyl, thienyl, (CH₂)ₘphenyl, (CH₂)ₘpyrrolyl, and (CH₂)ₘpyridyl; wherein said cycloalkyl groups optionally contain one or two heteroatoms selected independently from the group consisting of O, N, and S; wherein said alkyl, cycloalkyl, phenyl, pyrrolyl, and pyridyl groups are optionally substituted with one or two groups selected, independently, from halogen, methyl, ethyl, or trifluoromethyl; and wherein m is zero, 1, or 2; R′ is selected from the group consisting of H, halogen, phenyl, 2-(N,N-dimethylamino)ethyl, CF₃, OC₁-C₃ alkyl and C₁-C₃ alkyl; R₃ and R₄ vary independently, and are selected from the group consisting of H, halogen, CF₃, OCF₃, OC₁-C₃ alkyl, and C₁-C₃ alkyl; X is O or S; Y is O or S; q=1 or 0; R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₇)_{w}CH₂Ar; wherein w=0-3; Ar is selected from the group consisting of phenyl, pyrimidyl, or pyridyl, and a 5-member heteroaromatic ring; wherein said heteroaromatic ring contains 1 or 2 ring heteroatoms selected independently from the group consisting of N, O, and S; and R₆ is selected from the group consisting of H and methyl; wherein all cycloalkyl and cycloalkenyl groups in R₅ optionally contain one or two ring heteroatoms selected independently from the group consisting of N, O, and S; and wherein all alkyl, cycloalkyl, alkenyl, cycloalkenyl, heterocycloalkyl, heterocycloalkenyl, alkynyl, aryl, and heteroaryl groups in R₁, R₂, R₃, R₄, R₅, R₆, and Ar are optionally substituted with one or two substituents selected independently from the group consisting of C₁-C₃ alkyl, halogen, OEt, OMe, and trifluoromethyl.

In further embodiments, R₁ and R₂, vary independently, and are selected from the group consisting of H, halogen, CF₃, C₁-C₆ alkyl, C(═O)C₁-C₆ alkyl, C(═O)OC₁-C₆ alkyl, OC(═O)C₁-C₆ alkyl, OC₁-C₆ alkyl, SCH₃, (CH₂)ₘ cyclopropyl, (CH₂)ₘcyclobutyl, (CH₂)ₘ cyclopentyl, (CH₂)ₘ cyclohexyl, (CH₂)ₘoxazolyl, (CH₂)ₘisoxazolyl, (CH₂)ₘthiazolyl, (CH₂)ₘisothiazolyl, (CH₂)ₘphenyl, (CH₂)ₘpyrrolyl, (CH₂)ₘpyridyl, and (CH₂)ₘpyrimidyl; wherein said cyclopentyl and said cyclohexyl groups optionally contain one or two ring heteroatoms selected independently from the group consisting of O, N, and S; wherein said alkyl, cycloalkyl, phenyl, pyrrolyl, and pyridyl groups are optionally substituted with one or two groups selected, independently, from the group consisting of halogen, CH₃, ethyl, and CF₃; and m is zero, 1, or 2; R′ is selected from the group consisting of H, halogen, CF₃, and C₁-C₃ alkyl; R₃ and R₄ vary independently, and are selected from the group consisting of H, halogen, CF₃, OCF₃, OC₁-C₃ alkyl, and C₁-C₃ alkyl; X is O or S; Y is O; q=1 or 0; R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar; wherein w=0-3, Ar is selected from the group consisting of phenyl, pyridyl, and a 5-member heteroaromatic ring, wherein said heteroaromatic ring contains 1 or 2 ring heteroatoms selected independently from the group consisting of N, O, and S; R₆ is H or methyl; wherein all cycloalkyl and cycloalkenyl groups optionally contain one or two ring heteroatoms selected independently from the group consisting of N, O, and S; and wherein all alkyl, cycloalkyl, alkenyl, cycloalkenyl, heterocycloalkyl, heterocycloalkenyl, alkynyl, aryl, and heteroaryl groups in R₁, R₂, R₃, R₄, R₅, R₆, and Ar are optionally substituted with one or two substituents selected independently from the group consisting of C₁-C₃ alkyl, halogen, OMe, OEt, and CF₃.

In further embodiments, R₁ and R₂, vary independently, and are selected from the group consisting of H, halogen, CF₃, OC₁-C₃ alkyl, C₁-C₆ alkyl, C(═O)OC₁-C₃ alkyl, OC(═O)C₁-C₃ alkyl, and C(═O)C₁-C₃ alkyl; R′ is selected from the group consisting of H, F, CH₃, and ethyl; R₃ and R₄ vary independently, and are selected from the group consisting of H, F, Cl, CF₃, OCF₃, OC₁-C₃ alkyl, and C₁-C₃ alkyl; and R₅ is C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, or (CHR₆)_{w}Ar, CH₂(CHR₆)_{w}Ar, or (CHR₆)_{w}CH₂Ar.

In further embodiments, R₂ is H or F; R′ is H; R₃ is selected from the group consisting of H, CH₃, OCH₃, CF₃, OCF₃, and Cl; R₄ is selected from the group consisting of CH₃, OCH₃, CF₃, OCF₃, and Cl; and R₅ is C₃-C₆ alkyl or (CH₂)_{w}C₃-C₆ cycloalkyl.

In further embodiments, R₁ is halogen or CF₃; R₂ is H or F; R′ is H; R₃ and R₄ vary independently, and are selected from the group consisting of H, CH₃, OCH₃, CF₃, OCF₃, or Cl; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar.

In further embodiments, R₁ is halogen or CF₃; R₂ is H or F; R′ is H; R₃ and R₄ vary independently, and are selected from the group consisting of H, CH₃, OCH₃, CF₃, OCF₃, or Cl; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar.

### Formula 4

In an embodiment, the Kv7 channel activator may be selected from one of the following compounds according to Formula 4. Such compounds are described in U.S. Pat. No. 8,293,911 issued on Oct. 23, 2012 and corresponding to U.S. application Ser. No. 11/894,877; which is incorporated by reference in its entirety herein. In the case of any conflict of terminology in the context of Formula 4, this reference incorporated by reference herein controls.

In an embodiment, the Kv7 channel activator is a compound according to formula 4:

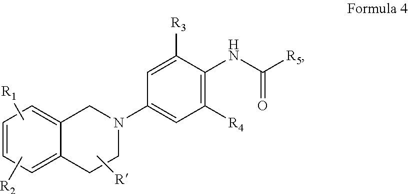

wherein, R₁ is selected from the group consisting of H, halogen, CN, CH₂CN, CF₃, C₁-C₆ alkyl, OCH₃, (C═O)OCH₃, O(C═O)CH₃, OCF₃, (CH₂)ₘC₃-C₆ cycloalkyl, phenyl, and pyridyl; R₂ is selected from the group consisting of H, F, OCH₃, CH₃, and CF₃; R₃ and R₄ vary independently, and are selected from the group consisting of H, F, Cl, CF₃, OCF₃, OC₁-C₃ alkyl, or C₁-C₃ alkyl; and R₅ is selected from the group consisting of C₁-C₆ alkyl, (CHR₆)_{w}C₃-C₆ cycloalkyl, (CHR₆)_{w}CH₂C₃-C₆ cycloalkyl, CH₂(CHR₆)_{w}C₃-C₆ cycloalkyl, CR₆═CH—C₃-C₆ cycloalkyl, CH═CR₆-C₃-C₆ cycloalkyl, (CHR₆)_{w}C₅-C₆ cycloalkenyl, CH₂(CHR₆)_{w}C₅-C₆ cycloalkenyl, C₂-C₆ alkenyl, C₂-C₆ alkynyl, Ar, (CHR₆)_{w}Ar, CH₂(CHR₆)_{w}Ar, and (CHR₆)_{w}CH₂Ar, wherein w=0-3; Ar is phenyl, furyl, pyrrolyl, oxazolyl, thiazolyl, thienyl, or pyridyl; and R₆ is C₁-C₃ alkyl; wherein all alkyl, cycloalkyl, aryl, and heteroaryl groups in R₁, R₂, R₃, R₄, R₅, R₆, and Ar are optionally substituted with one or two substituents selected independently from the group consisting of C₁-C₃ alkyl, halogen, OCH₃, OCH₂CH₃, CN, and CF₃.

In other embodiments, R₁ is selected from the group consisting of H, F, Cl, Br, CF₃, C₁-C₆ alkyl, OCH₃, CH₂OCH₃, CH₂CH₂OCH₃, CH₂OCH₂CH₃, and OCH₂CH₃; R′ is selected from the group consisting of H, CH₃, CH₂CH₃, or halogen; R₃ and R₄ vary independently, and are selected from the group consisting of H, F, Cl, CF₃, OCF₃, OCH₃, and CH₃; and R₅ is selected from the group consisting of C₁-C₆ alkyl, CH₂C₃-C₆ cycloalkyl, CH₂CH₂C₃-C₆ cycloalkyl, CH═CH—C₃-C₆ cycloalkyl, CH—CH—C₅-C₆ cycloalkenyl, CH₂C₅-C₆ cycloalkenyl, CH₂CH₂C₅-C₆ cycloalkenyl, C₂-C₆ alkenyl, and (CH₂)_{w}Ar; wherein w=1 or 2; Ar is selected from the group consisting of phenyl, oxazolyl, thiazolyl, isoxazolyl, isothiazolyl, furyl, thienyl, pyrrolyl, and pyridyl; wherein all alkyl, cycloalkyl, aryl, and heteroaryl groups in R₁, R₂, R₃, R₄, R₅, R₆, and Ar are optionally substituted with one or two substituents selected independently from the group consisting of CH₃, halogen, OCH₃, OCH₂CH₃, CN, and CF₃.

In other embodiments, R₁ is selected from the group consisting of H, F, Cl, Br, CF₃, C₁-C₆ alkyl, OCH₃, CH₂OCH₃, CH₂CH₂OCH₃, CH₂OCH₂CH₃, and OCH₂CH₃; R′ is selected from the group consisting of H, CH₃, CH₂CH₃, or halogen; R₃ and R₄ vary independently, and are selected from the group consisting of H, F, Cl, CF₃, OCF₃, OCH₃, and CH₃; and R₅ is selected from the group consisting of C₁-C₆ alkyl, CH₂C₃-C₆ cycloalkyl, CH₂CH₂C₃-C₆ cycloalkyl, CH═CH—C₃-C₆ cycloalkyl, CH═CH—C₅-C₆ cycloalkenyl, CH₂C₅-C₆ cycloalkenyl, CH₂CH₂C₅-C₆ cycloalkenyl, C₂-C₆ alkenyl, and (CH₂)_{w}Ar; wherein w=1 or 2; Ar is selected from the group consisting of phenyl, oxazolyl, thiazolyl, isoxazolyl, isothiazolyl, furyl, thienyl, pyrrolyl, and pyridyl; wherein all alkyl, cycloalkyl, aryl, and heteroaryl groups in R₁, R₂, R₃, R₄, R₅, R₆, and Ar are optionally substituted with one or two substituents selected independently from the group consisting of CH₃, halogen, OCH₃, OCH₂CH₃, CN, and CF₃. Wherein, R₁ is selected from the group consisting of F, CF₃, Cl, CH₃, CH₂CH₃, SCH₃, OCH₃, CH₂OCH₃, CH₂OCH₂CH₃, OCF₃, phenyl, thienyl, and H; R₂ is selected from the group consisting of H, F, Cl, and OCH₃; R′ is selected from the group consisting of H, F, CH₂CH₃, and CH₃; R₃ and R₄ vary independently, and are selected from the group consisting of H, Cl, CH₃, CF₃, OCH₃, and OCF₃; and R₅ is selected from the group consisting of C₄-C₆ alkyl, (CH₂)_{w}Ar, and (CH₂)_{w}C₅-C₆ cycloalkyl; wherein wis 1, 2, or 3.

In other embodiments, R₁ is selected from the group consisting of F, CF₃, Cl, CH₃, OCH₃, CH₂OCH₃, and H; R₂ is selected from the group consisting of H, F, CH₃, and Cl; R′ is H; R₃ is selected from the group consisting of H, Cl, CH₃, CF₃, OCH₃, and OCF₃; R₄ is selected from the group consisting of Cl, OCH₃, and CH₃; and R₅ is C₄-C₆ alkyl or 2-cyclopentyl ethyl.

In other embodiments, R₃ and R₄ are both CH₃ or both OCH₃; and R₅ is C₅-C₆ alkyl.

In other embodiments, R′ and R₂ are H; R₃ and R₄ are both methyl; and R₅ is C₅-C₆ alkyl or (CH₂)_{w}C₅-C₆ cycloalkyl; wherein wis 1, 2, or 3.

In other embodiments, the compound is selected from the group consisting of: N-(2-chloro-4-(3,4-dihydroisoquinolin-2 (1H)-yl)-6-(trifluoromethyl)phenyl)-3,3-dimethylbutanamide; N-(4-(3,4-dihydroisoquinolin-2 (1H)-yl)-2,6-dimethylphenyl)-3,3-dimethylbutanamide; N-(2-chloro-4-(3,4-dihydroisoquinolin-2 (1H)-yl)-6-(trifluoromethyl)phenyl)-3-cyclopentylpropanamide; N-(2-chloro-4-(6-fluoro-3,4-dihydroisoquinolin-2 (1H)-yl)-6-(trifluoromethylphenyl)-3,3-dimethylbutanamide; N-[2-chloro-4-(3,4-dihydro-1H-isoquinolin-2-yl)-6-methyl phenyl]-3,3-dimethylbutanamide; N-[2-chloro-4-(6-fluoro-3,4-dihydro-1H-isoquinolin-2-yl)-6-trifluoromethyl phenyl]-3-cyclopentylpropionamide; N-[2,6-dimethyl-4-(6-trifluoromethyl-3,4-dihydro-1H-isoquinolin-2-yl)-phenyl]-3,3-dimethylbutanamide; N-[2-chloro-6-trifluoromethyl-4-(6-trifluoromethyl-3,4-dihydro-1H-isoquinolin-2-yl)-phenyl]-3,3-dimethylbutanamide; N-[2-chloro-4-(6-chloro-3,4-dihydro-1H-isoquinolin-2-yl)-6-trifluoromethyl phenyl]-3,3-dimethylbutanamide; N-[4-(6-chloro-3,4-dihydro-1H-isoquinolin-2-yl)-2,6-dimethyl-phenyl]-3,3-dimethylbutanamide; N-[4-(6-fluoro-3,4-dihydro-1H-isoquinolin-2-yl)-2,6-dimethyl phenyl]-3,3-dimethylbutanamide; N-[2-chloro-4-(7-fluoro-3,4-dihydro-1H-isoquinolin-2-yl)-6-trifluoromethyl-phenyl]-3,3-dimethylbutanamide; N-[4-(7-fluoro-3,4-dihydro-1H-isoquinolin-2-yl)-2,6-dimethyl-phenyl]-3,3-dimethylbutanamide; N-[2-chloro-4-(6-fluoro-3,4-dihydro-1H-isoquinolin-2-yl)-6-methylphenyl]-3,3-dimethylbutanamide; N-[2-chloro-4-(7-fluoro-3,4-dihydro-1H-isoquinolin-2-yl)-6-methylphenyl]-3,3-dimethylbutanamide; N-[2-chloro-6-methyl-4-(6-trifluoromethyl-3,4-dihydro-1H-isoquinolin-2-yl)-phenyl]-3,3-dimethylbutanamide; N-[2-chloro-4-(6-chloro-3,4-dihydro-1H-isoquinolin-2-yl)-6-methyl-phenyl]-3,3-dimethylbutanamide; N-[2-chloro-4-(6-fluoro-3,4-dihydro-1H-isoquinolin-2-yl)-phenyl]-3,3-dimethylbutanamide; N-[4-(6-fluoro-3,4-dihydro-1H-isoquinolin-2-yl)-2-methyl-phenyl]-3,3-dimethylbutanamide; N-[4-(6-fluoro-3,4-dihydro-1H-isoquinolin-2-yl)-2-trifluoromethylphenyl]-3,3-dimethylbutanamide; N-[2-chloro-4-(6-trifluoromethyl-3,4-dihydro-1H-isoquinolin-2-yl)-phenyl]-3,3-dimethylbutanamide; N-[4-(7-fluoro-3,4-dihydro-1H-isoquinolin-2-yl)-2-trifluoromethyl-phenyl]-3,3-dimethylbutanamide; 3,3-dimethyl-N-[2-trifluoromethyl-4-(7-trifluoromethyl-3,4-dihydro-1H-isoquinolin-2-yl)-phenyl]butanamide; N-[4-(6-methoxy-3,4-dihydro-1H-isoquinolin-2-yl)-2,6-dimethyl-phenyl]-3,3-dimethylbutanamide; N-[4-(3,4-dihydro-1H-isoquinolin-2-yl)-2-methoxy-6-methyl-phenyl]-3,3-dimethylbutanamide; N-[2-chloro-4-(3,4-dihydro-1H-isoquinolin-2-yl)-6-trifluoromethoxy-phenyl]-3,3-dimethylbutanamide; N-[4-(3,4-dihydro-M-isoquinolin-2-yl)-2,6-dimethoxy-phenyl]-3,3-dimethylbutanamide; N-[2,6-dimethyl-4-(7-trifluoromethyl-3,4-dihydro-1H-isoquinolin-2-yl)-phenyl]-3,3-dimethyl butanamide; N-[2,6-Dimethyl-4-(6-trifluoromethyl-3,4-dihydro-1H-isoquinolin-2-yl)-phenyl]-3,3-dimethyl-thiobutanamide; [2,6-Dimethyl-4-(6-trifluoromethyl-3,4-dihydro-1H-isoquinolin-2-yl)-phenyl]-carbamic acid ethyl ester; and N-[2,6-Dimethyl-4-(7-trifluoromethyl-3,4-dihydro-1H-isoquinolin-2-yl)-phenyl]-3,3-dimethyl butanamide.

### Formula 5

In another embodiment, the Kv7 channel activator may be selected from one of the following compounds. Such compounds are described in U.S. Pat. No. 8,993,593 issued on Mar. 31, 2015 and corresponding to U.S. application Ser. No. 12/698,070 filed Feb. 1, 2010; US Publication No. US20220265634A1, published Aug. 25, 2022 and corresponding to U.S. application Ser. No. 17/668,340 filed Feb. 9, 2022; which are incorporated by reference in their entirety herein. In the case of any conflict of terminology in the context of Formula 5, these references incorporated by reference herein control.

In an embodiment, the Kv7 channel activator is a compound according to formula 5:

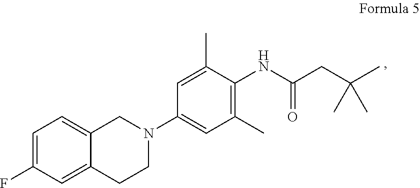 [SMILES: Cc1cc(N2CCc3cc(F)ccc3C2)cc(C)c1NC(=O)CC(C)(C)C]

optionally wherein the compound is substituted at any position.

### Formula 6

In another embodiment, the Kv7 channel activator may be the following compound (ezogabine, also known as retigabine) or a pharmaceutically acceptable salt thereof. Ezogabine is a compound according to formula 6: In the case of any conflict of terminology in the context of Formula 6, these references incorporated by reference herein control.

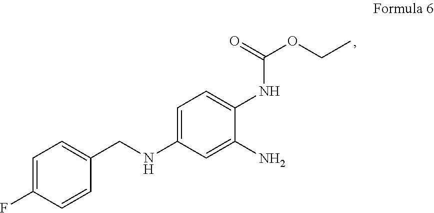 [SMILES: CCOC(=O)Nc1ccc(NCc2ccc(F)cc2)cc1N]

or, optionally, wherein the compound is substituted at any position.

### Formula 7

In an embodiment, the Kv7 channel activator may be selected from one of the following compounds according to Formula 7. Such compounds are described in U.S. Pat. No. 10,526,328 issued on Jan. 7, 2020 and corresponding to U.S. application Ser. No. 16/124,853 filed Sep. 7, 2018; U.S. Pat. No. 10,106,536 issued on Oct. 23, 2018 and corresponding to U.S. application Ser. No. 15/591,844 filed on May 10, 2017; U.S. Pat. No. 9,650,376 issued on May 16, 2017, and corresponding to U.S. application Ser. No. 14/776,271 filed Mar. 17, 2014; US Publication No. US20220060208A1, published Feb. 24, 2022 and corresponding to U.S. application Ser. No. 17/277,145 filed Jan. 14, 2019; which are incorporated by reference in their entirety herein. In the case of any conflict of terminology in the context of Formula 7, these references incorporated by reference herein control.

In an embodiment, the Kv7 channel activator is a compound according to formula 7:

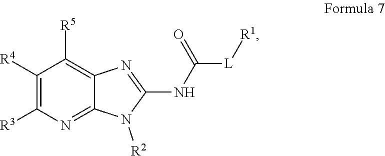

wherein, L is CH₂; R¹ is optionally substituted cyclic C₃H₅, wherein the optional substituent of R¹ is CF₃; R² is optionally substituted cyclobutyl; R³ is optionally substituted C₃ alkyl, wherein the optional substituent of R³ is OH; R⁴ is H; and R⁵ is H;
   or
   wherein, L is CH₂; R¹ is optionally substituted C₂ alkyl, wherein the optional substituents of R¹ are independently CF₃ or CH₃; R² is optionally substituted cyclobutyl; R³ is optionally substituted C₃ alkyl, wherein the optional substituent of R³ is OH; R⁴ is H; and R⁵ is H;
   or
   wherein, wherein L is CH₂, CF₂, CHCH₃, CH₂CH₂, C₃H₆, CH₂O, C₂H₄O, or C₃H₆O with the O of CH₂O, C₂H₄O, or C₃H₆O bonded with R¹; wherein R¹ is optionally substituted C₁₋₂ alkyl, optionally substituted C₅-10 cycloalkyl, optionally substituted C₁₋₁₂-O-alkyl, optionally substituted C₆-10 aryl, optionally substituted C₆-10-O-aryl, or optionally substituted C₂₋₉ heterocyclyl, wherein the optional substituents of R¹ are independently R^{A}, F, Cl, CN, OR^{A}, CF₃, NR^{A}R^{B}, COR^{A}, CO₂R^{A}, OCOR^{A}, NR^{A}COR^{B}, CONR^{A}R^{B}, wherein R^{A} and R^{B} are independently H or C₁₋₁₂ alkyl; wherein R² is optionally substituted C₂₋₄ acyclic alkyl, optionally substituted cyclobutyl, optionally substituted C₆-10 aryl, or optionally substituted C₂₋₉ heterocyclyl, wherein the optional substituents of R² are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ alkylthio, or C₁₋₆ alkylsulfonyl; wherein R³, R⁴, and R⁵ are independently H, F, Cl, Br, I, CN, optionally substituted C₁₋₁₂ alkyl, optionally substituted C₁₋₁₂-O-alkyl, optionally substituted C₂-9 heterocyclyl, optionally substituted C₆-10 aryl, optionally substituted C₂₋₉-O-heterocyclyl, optionally substituted C₆-10 O-aryl, optionally substituted C₁₋₁₂ acylamino, optionally substituted C₁₋₁₂ aminoacyl, or optionally substituted C₁₋₁₂ aminoalkyl, wherein the optional substituents of R³, R⁴, or R⁵ are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ acylamino, C₁₋₆ alkylthio, or C₁₋₆ alkylsulfonyl.

In further embodiments, R² is optionally substituted C₄H₉, optionally substituted cyclobutyl, optionally substituted C₆-10 aryl, or optionally substituted C₂₋₉ heterocyclyl.

In further embodiments, R² is optionally substituted C₄H₉, or optionally substituted cyclobutyl.

In further embodiments, R² is optionally substituted phenyl, or optionally substituted C₂₋₉ heterocyclyl.

In further embodiments, R¹ is optionally substituted phenyl.

In further embodiments, R¹ is optionally substituted phenyl.

In further embodiments, R¹ is optionally substituted cyclopentyl or optionally substituted cyclohexyl.

In further embodiments, R¹ is C₂₋₄-O-alkyl.

In further embodiments, R¹ is optionally substituted tetrahydrofuranyl or optionally substituted tetrahydro-2H-pyranyl.

In further embodiments, R¹ is selected from the group consisting of:

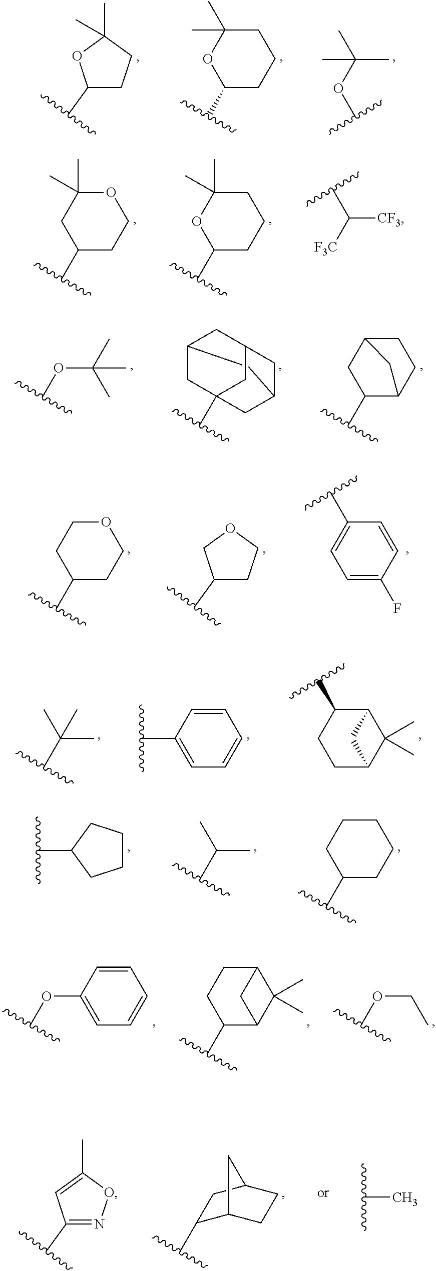

In further embodiments, R² is selected from the group consisting of:

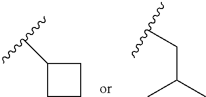

In further embodiments, R³ is CF₃, Cl, CN, OCH₃, or H.

In further embodiments, R¹ is selected from the group consisting of:

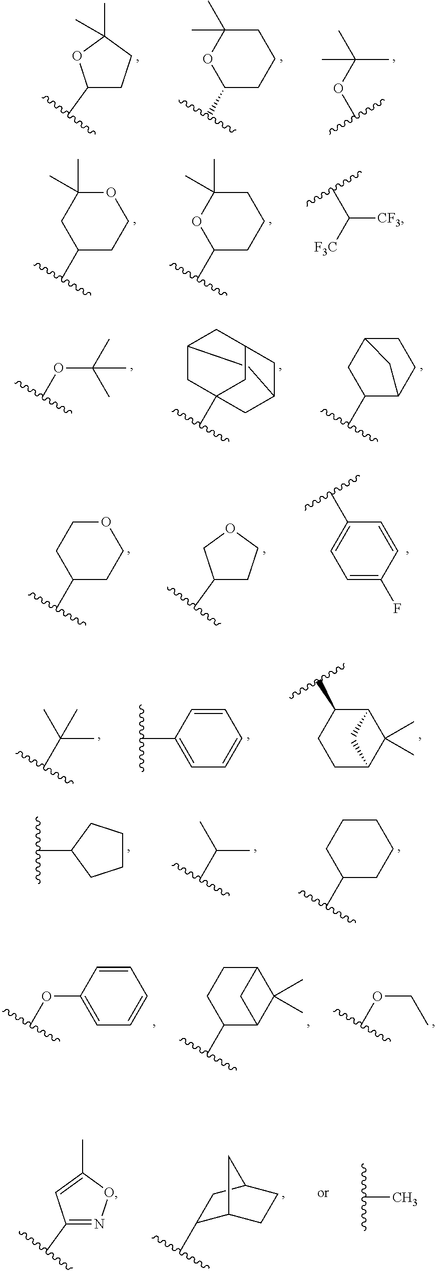

In further embodiments, R⁴ is CH₃, CF₃, Cl, or H.

In further embodiments, R² is selected from the group consisting of:

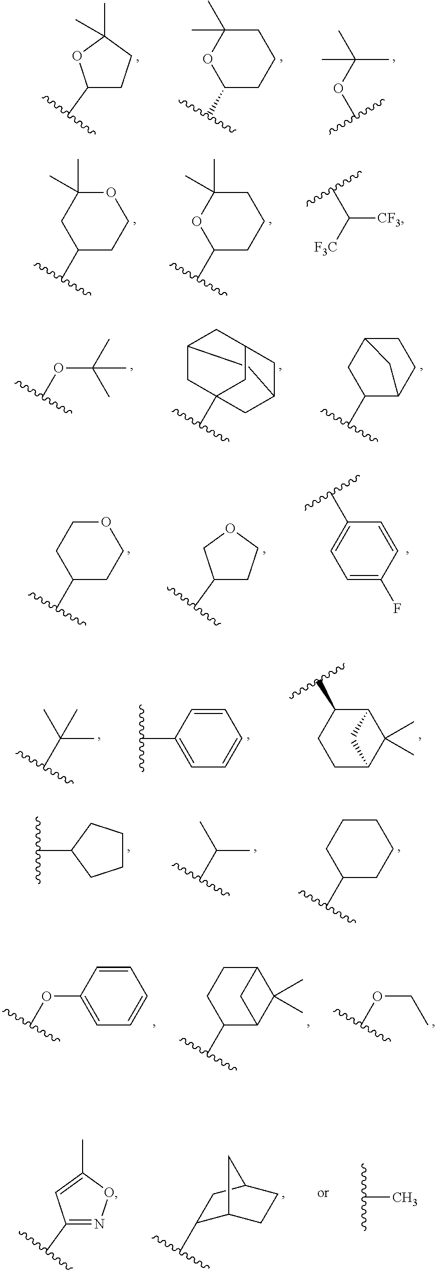

In further embodiments, the Kv7 channel activator is selected from the group consisting of:

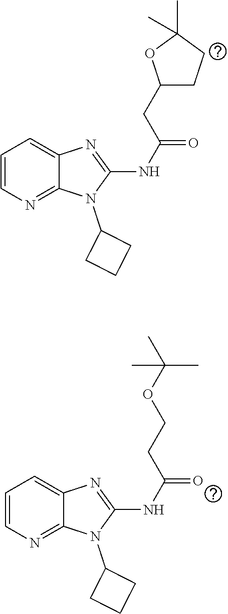 [SMILES: CC1(C)CCC(CC(=O)Nc2nc3cccnc3n2C2CCC2)O1 ; CC(C)(C)OCCC(=O)Nc1nc2cccnc2n1C1CCC1]

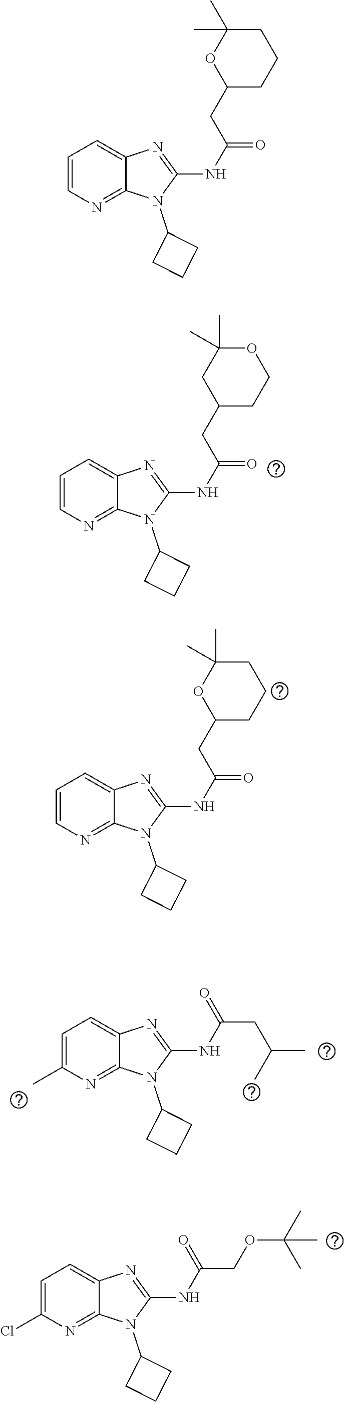 [SMILES: CC1(C)CCCC(CC(=O)Nc2nc3cccnc3n2C2CCC2)O1 ; CC1(C)CC(CC(=O)Nc2nc3cccnc3n2C2CCC2)CCO1 ; CC1(C)CCCC(CC(=O)Nc2nc3cccnc3n2C2CCC2)O1 ; Cc1ccc2nc(NC(=O)CC(C)C)n(C3CCC3)c2n1 ; CC(C)(C)OCC(=O)Nc1nc2ccc(Cl)nc2n1C1CCC1]

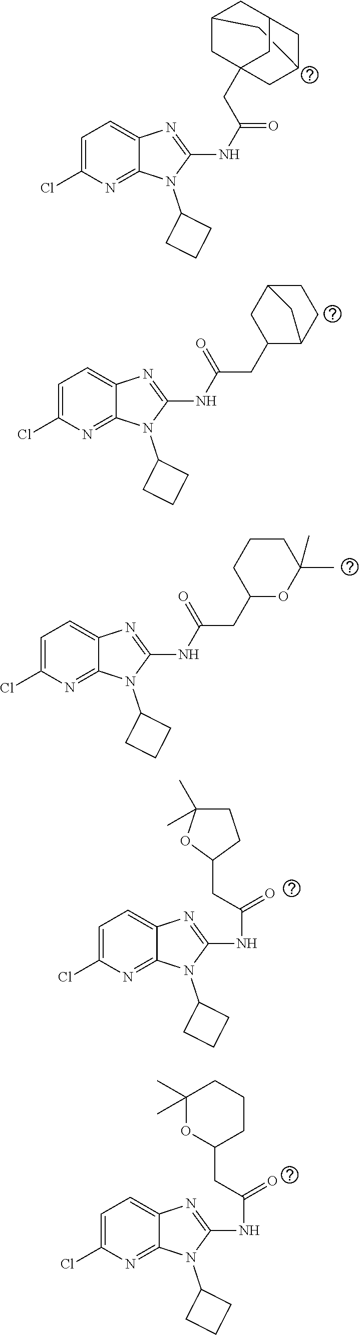

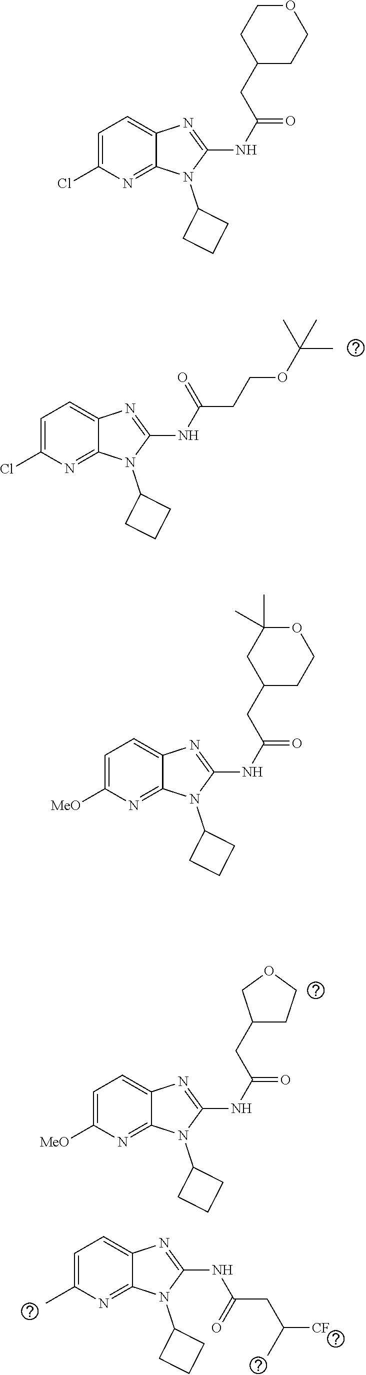

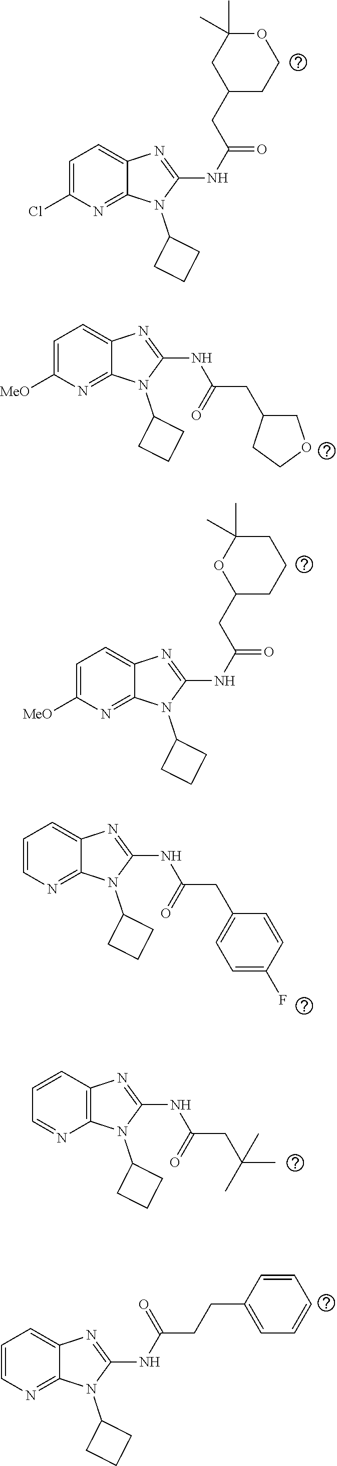 [SMILES: CC1(C)CC(CC(=O)Nc2nc3ccc(Cl)nc3n2C2CCC2)CCO1 ; COc1ccc2nc(NC(=O)CC3CCOC3)n(C3CCC3)c2n1 ; COc1ccc2nc(NC(=O)CC3CCCC(C)(C)O3)n(C3CCC3)c2n1 ; O=C(Cc1ccc(F)cc1)Nc1nc2cccnc2n1C1CCC1 ; CC(C)(C)CC(=O)Nc1nc2cccnc2n1C1CCC1 ; O=C(CCc1ccccc1)Nc1nc2cccnc2n1C1CCC1]

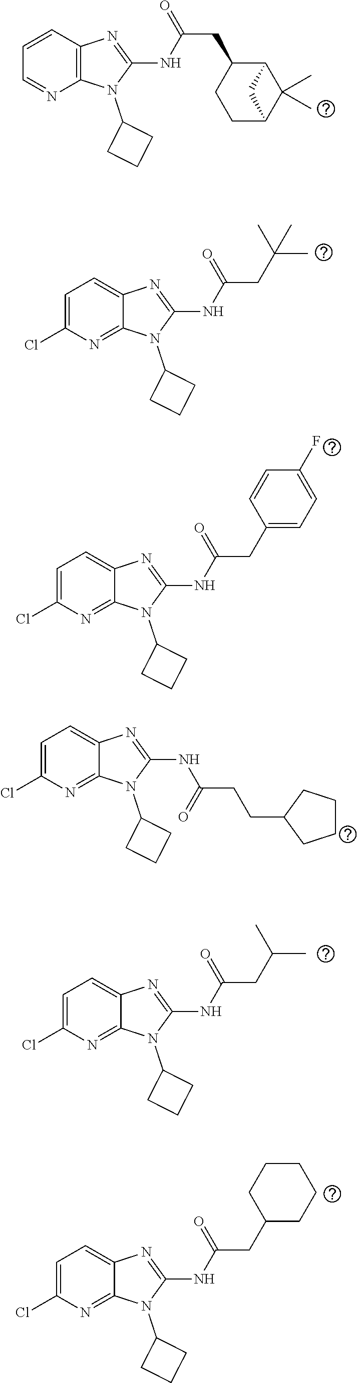 [SMILES: CC1(C)[C@H]2CC[C@@H](CC(=O)Nc3nc4cccnc4n3C3CCC3)[C@@H]1C2 ; CC(C)(C)CC(=O)Nc1nc2ccc(Cl)nc2n1C1CCC1 ; O=C(Cc1ccc(F)cc1)Nc1nc2ccc(Cl)nc2n1C1CCC1 ; O=C(CCC1CCCC1)Nc1nc2ccc(Cl)nc2n1C1CCC1 ; CC(C)CC(=O)Nc1nc2ccc(Cl)nc2n1C1CCC1 ; O=C(CC1CCCCC1)Nc1nc2ccc(Cl)nc2n1C1CCC1]

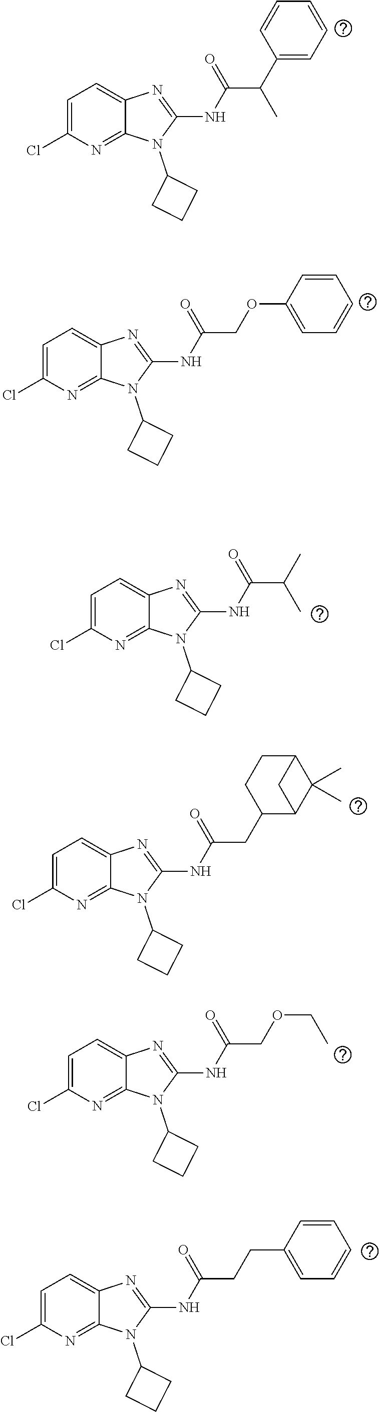 [SMILES: CC(C(=O)Nc1nc2ccc(Cl)nc2n1C1CCC1)c1ccccc1 ; O=C(COc1ccccc1)Nc1nc2ccc(Cl)nc2n1C1CCC1 ; CC(C)C(=O)Nc1nc2ccc(Cl)nc2n1C1CCC1 ; CC1(C)C2CCC(CC(=O)Nc3nc4ccc(Cl)nc4n3C3CCC3)C1C2 ; CCOCC(=O)Nc1nc2ccc(Cl)nc2n1C1CCC1 ; O=C(CCc1ccccc1)Nc1nc2ccc(Cl)nc2n1C1CCC1]

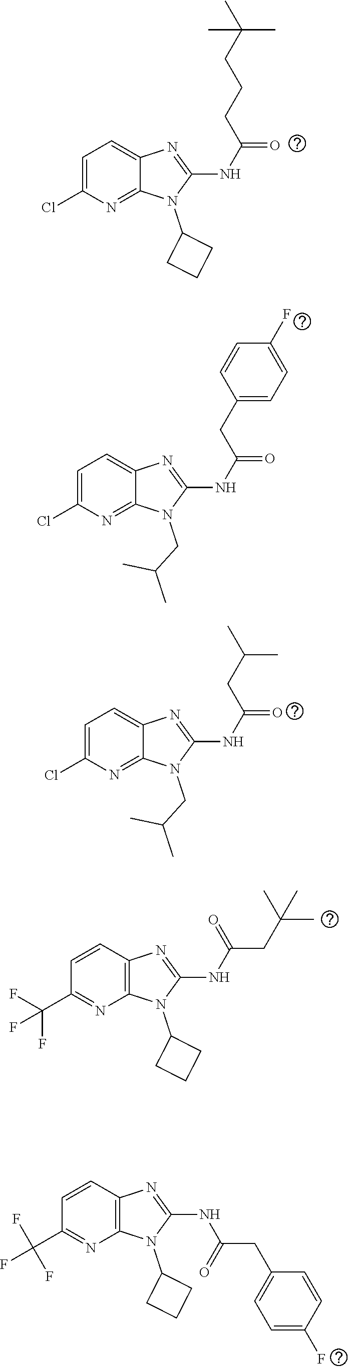 [SMILES: CC(C)(C)CCCC(=O)Nc1nc2ccc(Cl)nc2n1C1CCC1 ; CC(C)Cn1c(NC(=O)Cc2ccc(F)cc2)nc2ccc(Cl)nc21 ; CC(C)CC(=O)Nc1nc2ccc(Cl)nc2n1CC(C)C ; CC(C)(C)CC(=O)Nc1nc2ccc(C(F)(F)F)nc2n1C1CCC1 ; O=C(Cc1ccc(F)cc1)Nc1nc2ccc(C(F)(F)F)nc2n1C1CCC1]

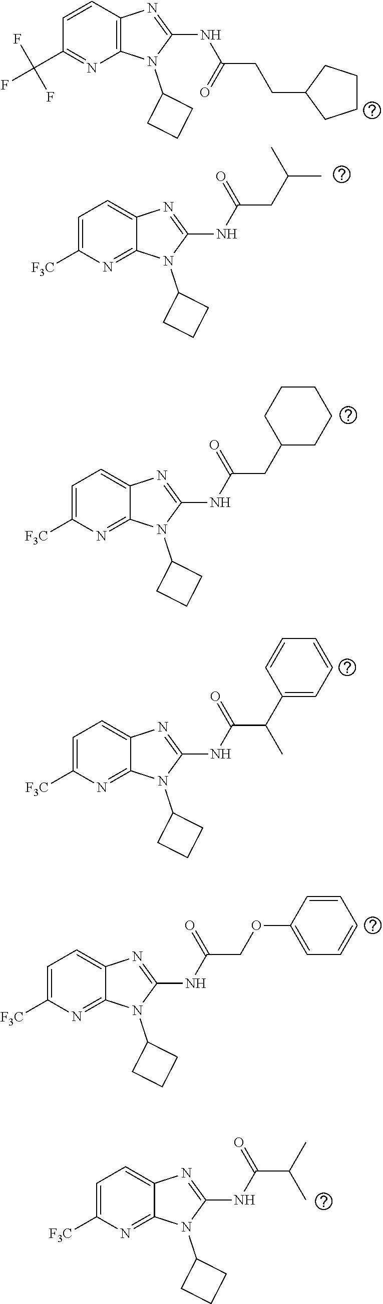 [SMILES: O=C(CCC1CCCC1)Nc1nc2ccc(C(F)(F)F)nc2n1C1CCC1 ; CC(C)CC(=O)Nc1nc2ccc(C(F)(F)F)nc2n1C1CCC1 ; O=C(CC1CCCCC1)Nc1nc2ccc(C(F)(F)F)nc2n1C1CCC1 ; CC(C(=O)Nc1nc2ccc(C(F)(F)F)nc2n1C1CCC1)c1ccccc1 ; O=C(COc1ccccc1)Nc1nc2ccc(C(F)(F)F)nc2n1C1CCC1 ; CC(C)C(=O)Nc1nc2ccc(C(F)(F)F)nc2n1C1CCC1]

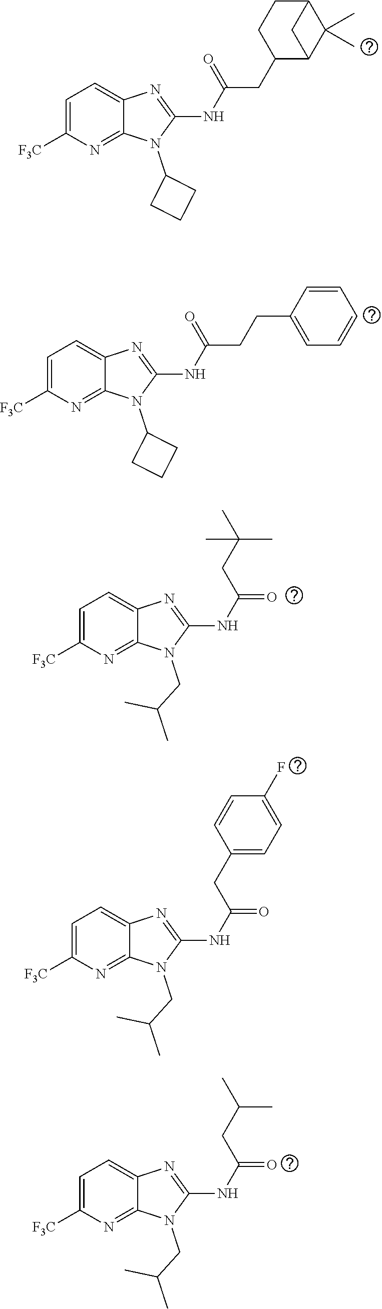 [SMILES: CC1(C)C2CCC(CC(=O)Nc3nc4ccc(C(F)(F)F)nc4n3C3CCC3)C1C2 ; O=C(CCc1ccccc1)Nc1nc2ccc(C(F)(F)F)nc2n1C1CCC1 ; CC(C)Cn1c(NC(=O)CC(C)(C)C)nc2ccc(C(F)(F)F)nc21 ; CC(C)Cn1c(NC(=O)Cc2ccc(F)cc2)nc2ccc(C(F)(F)F)nc21 ; CC(C)CC(=O)Nc1nc2ccc(C(F)(F)F)nc2n1CC(C)C]

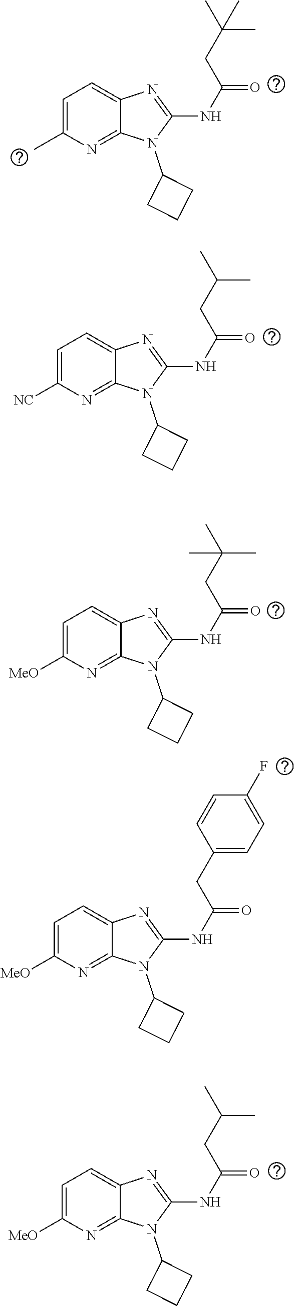

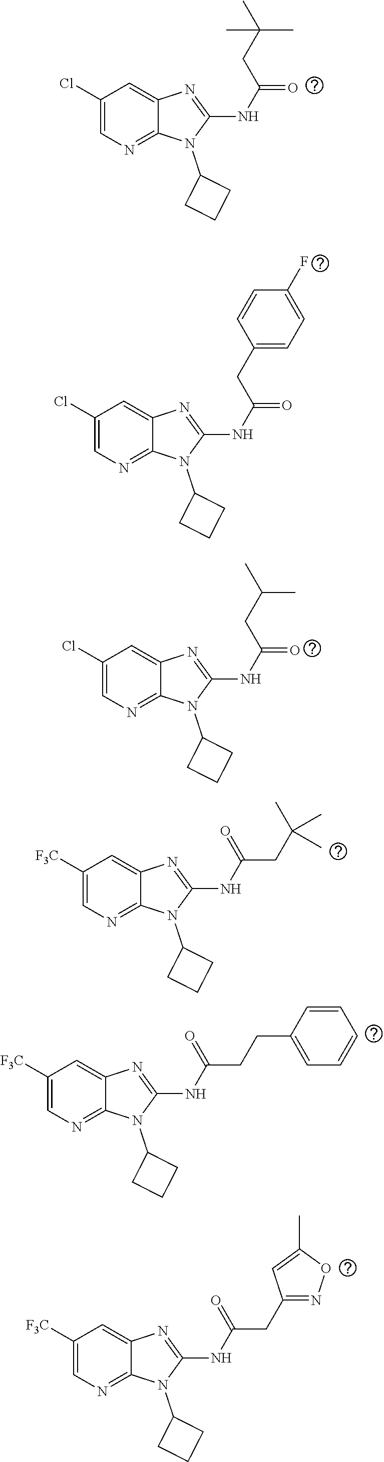

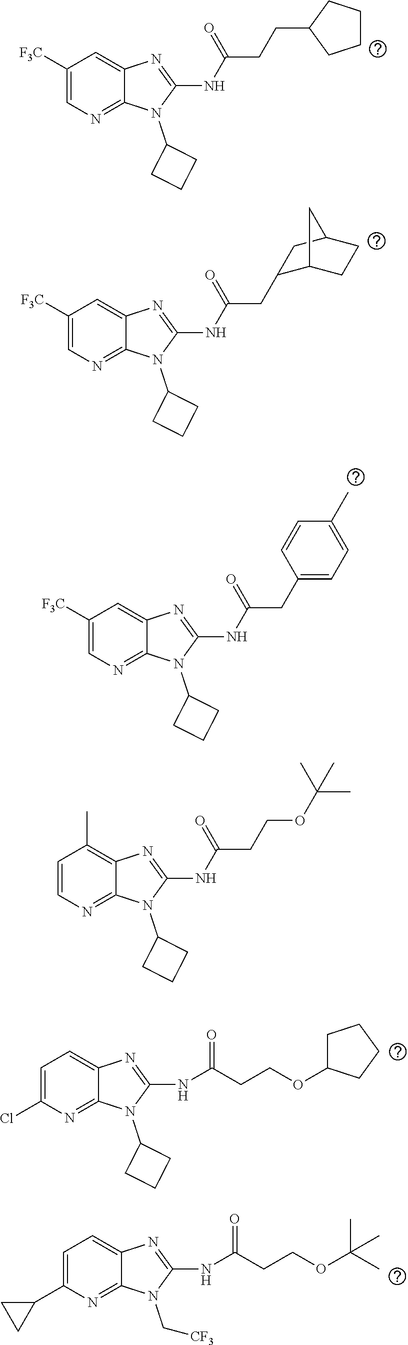 [SMILES: O=C(CCC1CCCC1)Nc1nc2cc(C(F)(F)F)cnc2n1C1CCC1 ; O=C(CC1CC2CCC1C2)Nc1nc2cc(C(F)(F)F)cnc2n1C1CCC1 ; Cc1ccc(CC(=O)Nc2nc3cc(C(F)(F)F)cnc3n2C2CCC2)cc1 ; Cc1ccnc2c1nc(NC(=O)CCOC(C)(C)C)n2C1CCC1 ; O=C(CCOC1CCCC1)Nc1nc2ccc(Cl)nc2n1C1CCC1 ; CC(C)(C)OCCC(=O)Nc1nc2ccc(C3CC3)nc2n1CC(F)(F)F]

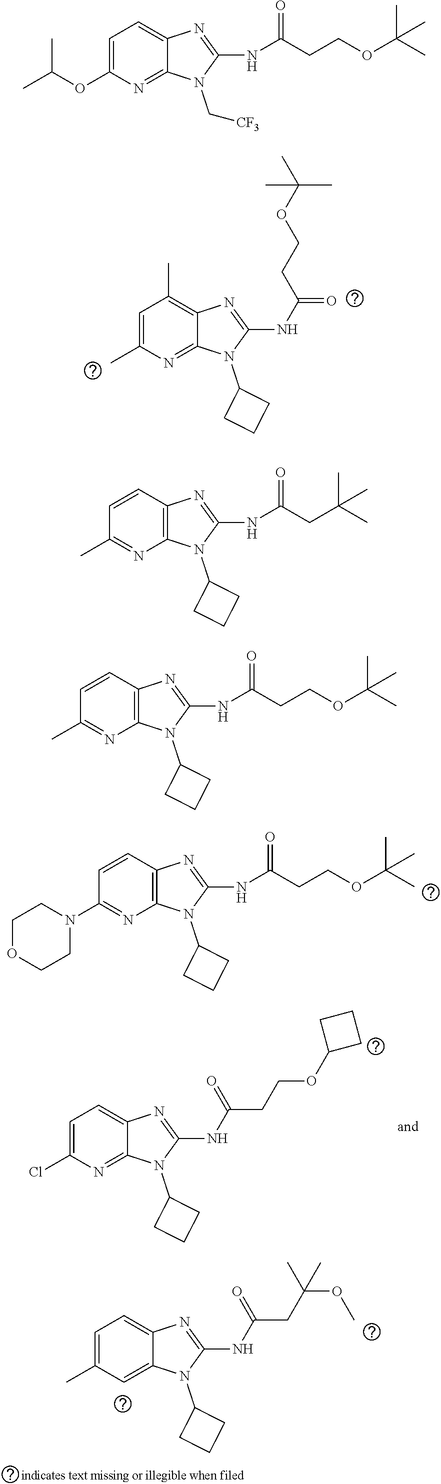

In further embodiments, the Kv7 channel activator is selected from the group consisting of:

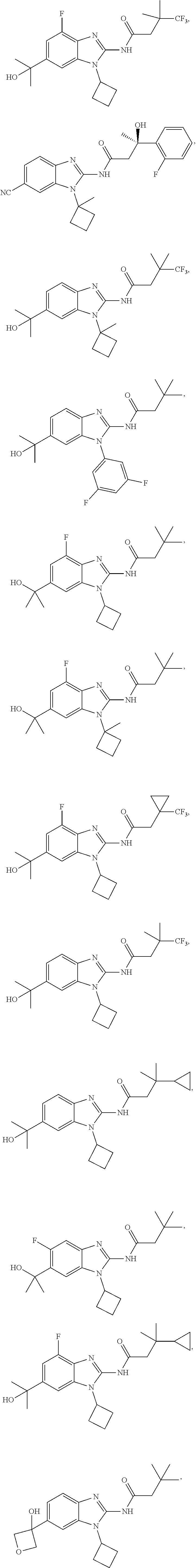

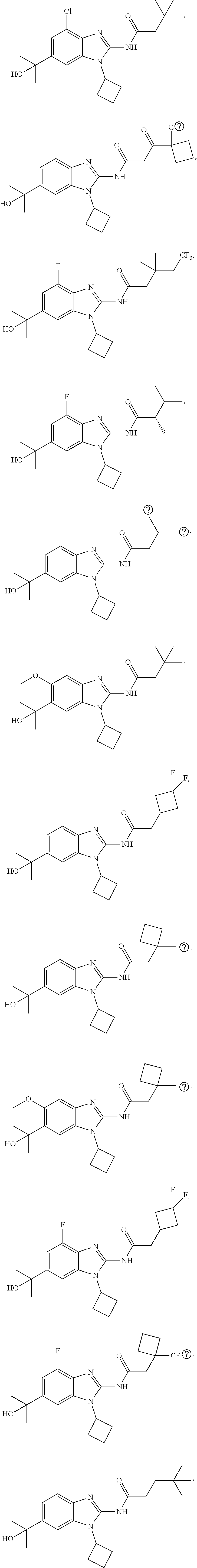

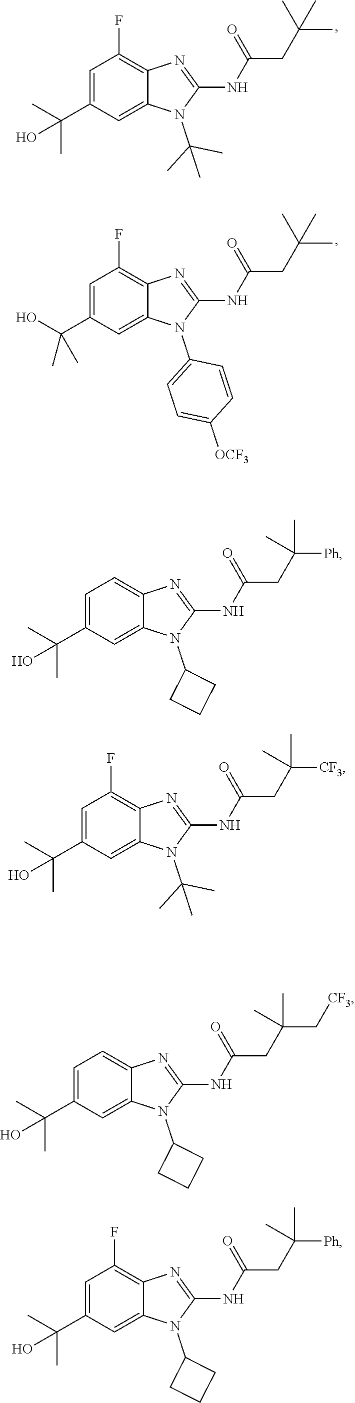

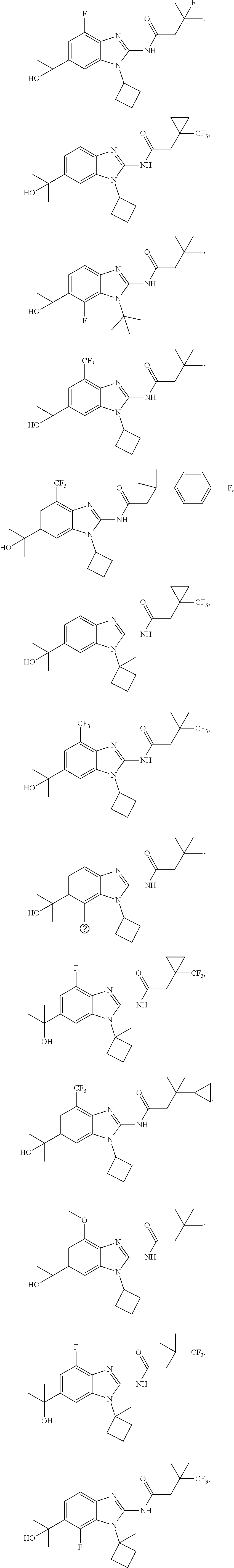

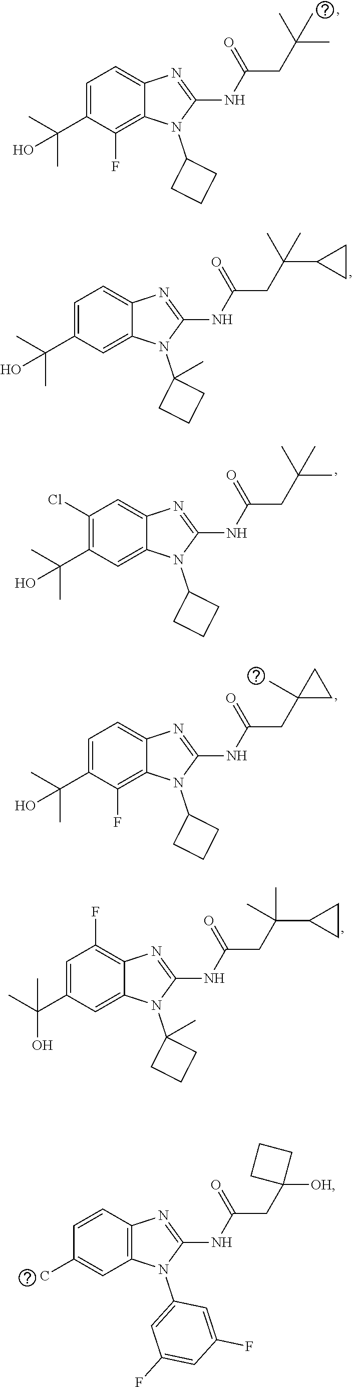

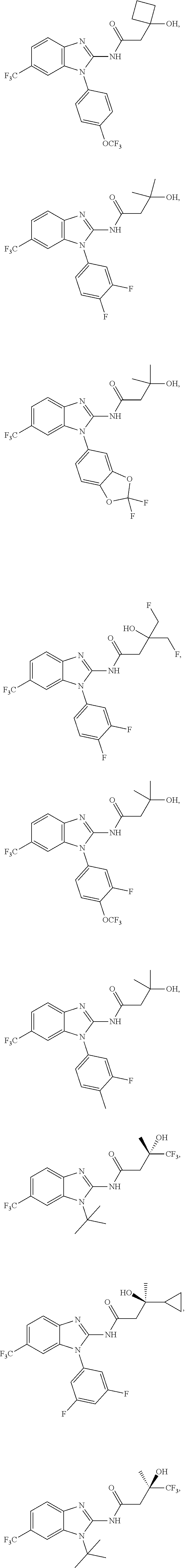

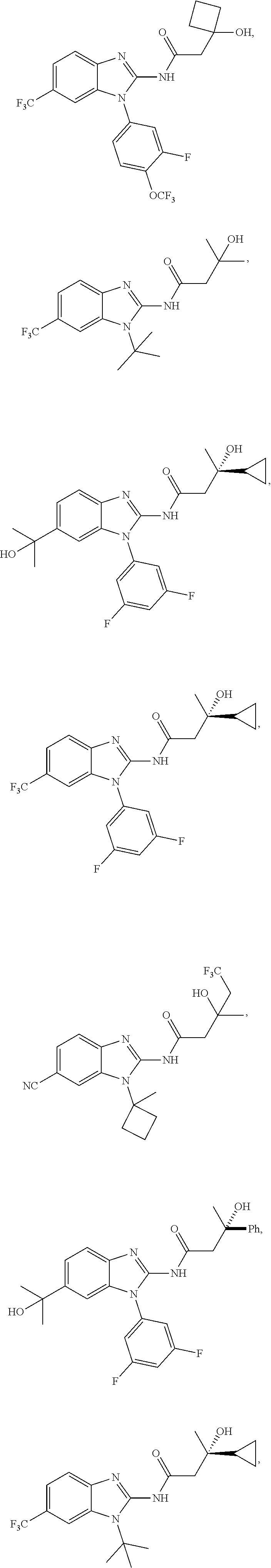

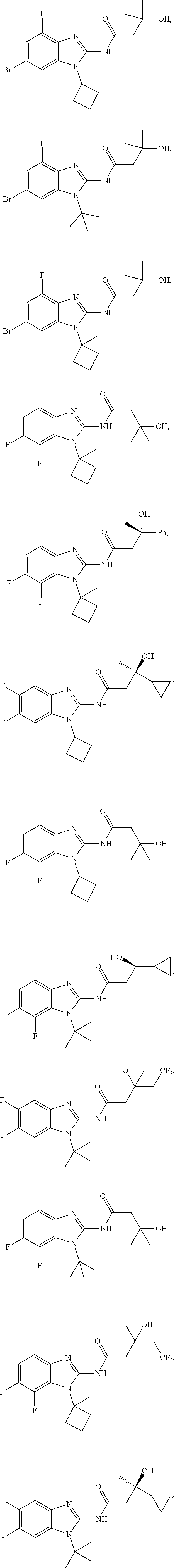

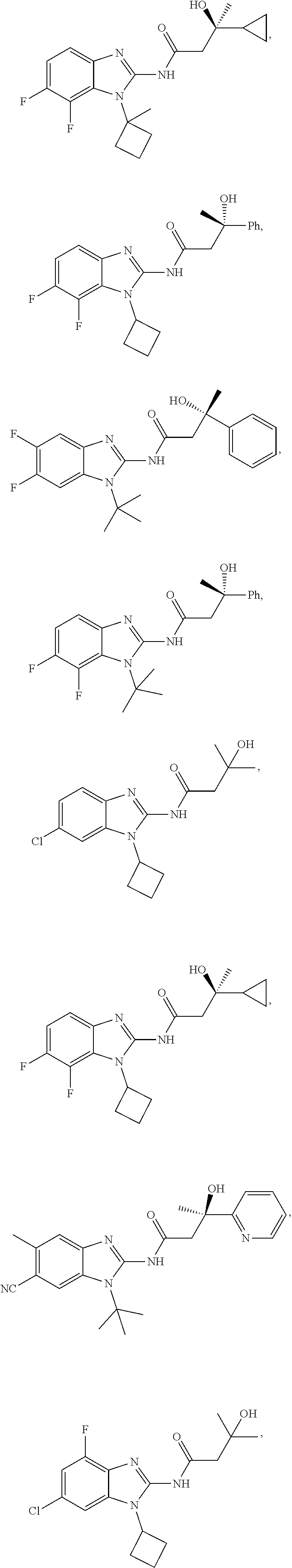

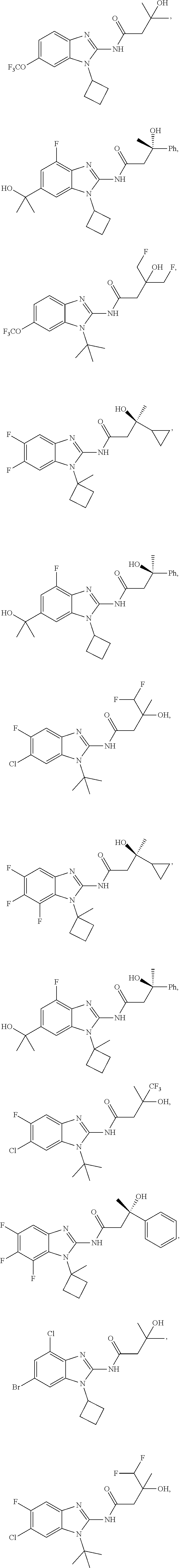

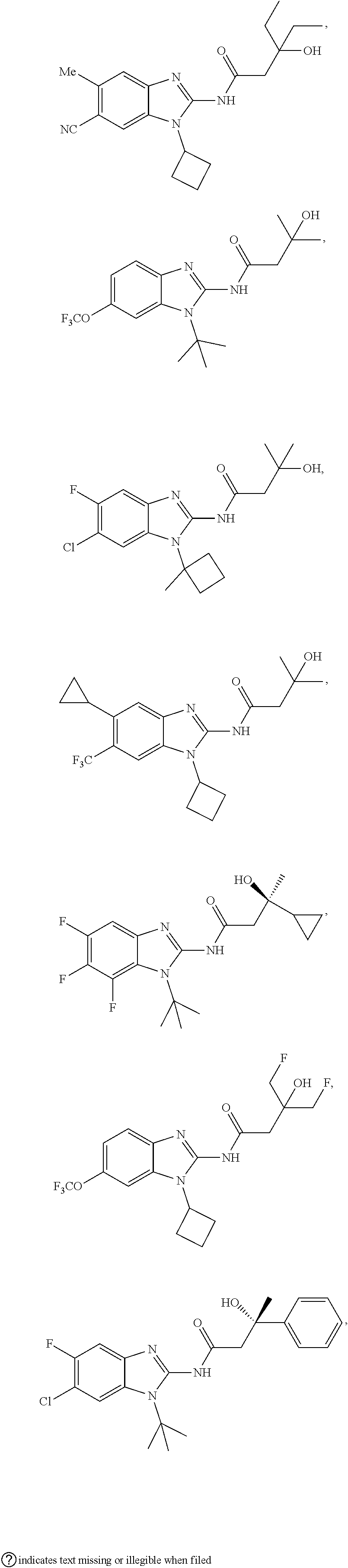

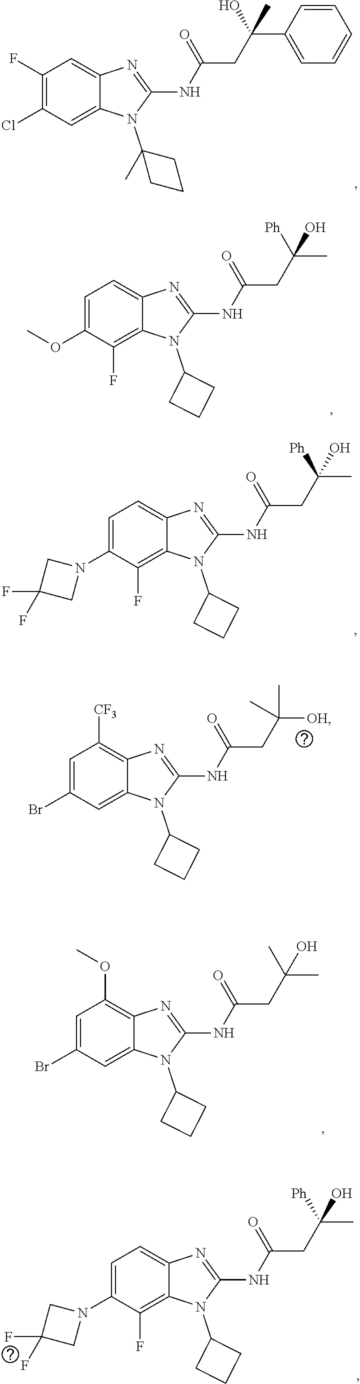 [SMILES: CC1(n2c(NC(=O)C[C@](C)(O)c3ccccc3)nc3cc(F)c(Cl)cc32)CCC1 ; COc1ccc2nc(NC(=O)C[C@](C)(O)c3ccccc3)n(C3CCC3)c2c1F ; C[C@@](O)(CC(=O)Nc1nc2ccc(N3CC(F)(F)C3)c(F)c2n1C1CCC1)c1ccccc1 ; CC(C)(O)CC(=O)Nc1nc2c(C(F)(F)F)cc(Br)cc2n1C1CCC1 ; COc1cc(Br)cc2c1nc(NC(=O)CC(C)(C)O)n2C1CCC1 ; C[C@](O)(CC(=O)Nc1nc2ccc(N3CC(F)(F)C3)c(F)c2n1C1CCC1)c1ccccc1]

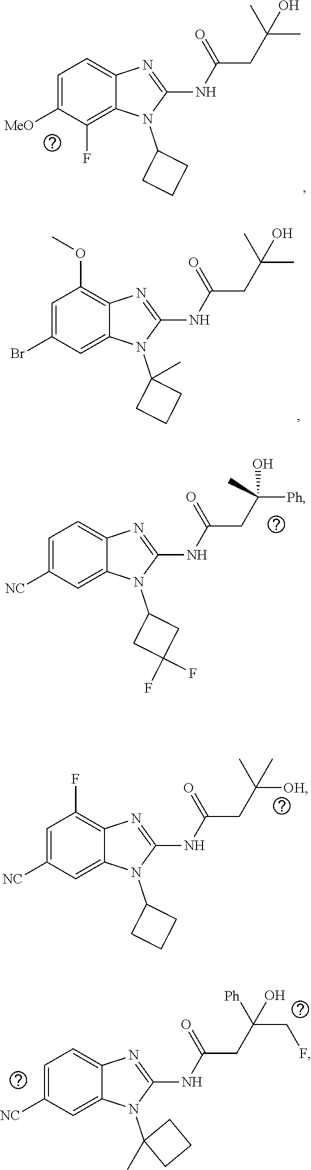 [SMILES: COc1ccc2nc(NC(=O)CC(C)(C)O)n(C3CCC3)c2c1F ; COc1cc(Br)cc2c1nc(NC(=O)CC(C)(C)O)n2C1(C)CCC1 ; C[C@](O)(CC(=O)Nc1nc2ccc(C#N)cc2n1C1CC(F)(F)C1)c1ccccc1 ; CC(C)(O)CC(=O)Nc1nc2c(F)cc(C#N)cc2n1C1CCC1 ; CC1(n2c(NC(=O)CC(O)(CF)c3ccccc3)nc3ccc(C#N)cc32)CCC1]

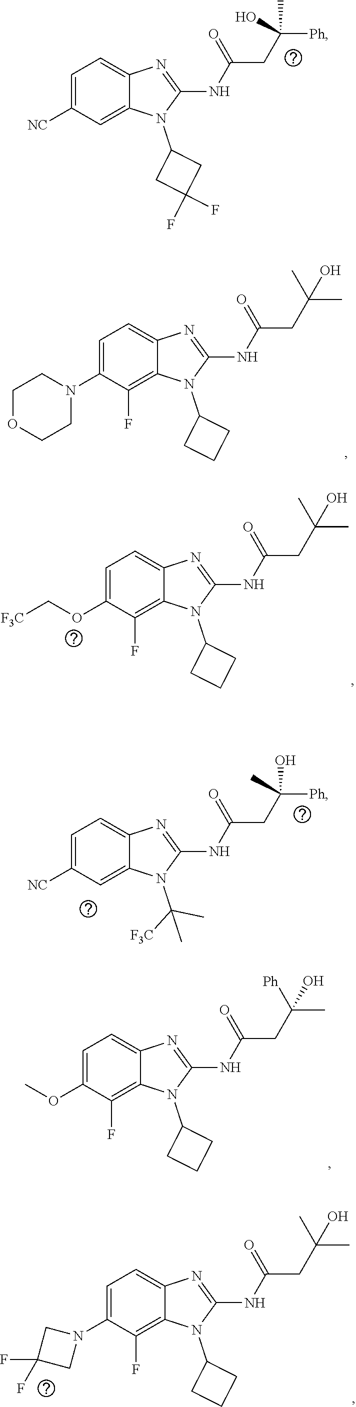

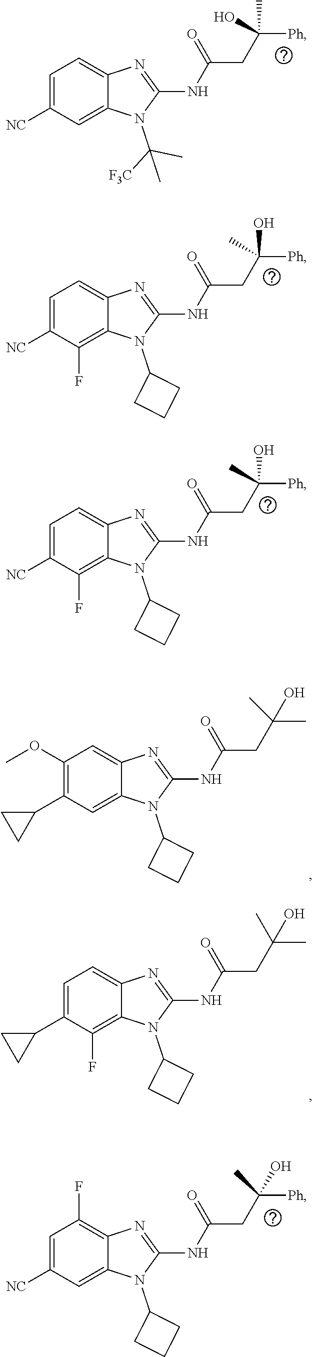 [SMILES: CC(C)(n1c(NC(=O)C[C@@](C)(O)c2ccccc2)nc2ccc(C#N)cc21)C(F)(F)F ; C[C@@](O)(CC(=O)Nc1nc2ccc(C#N)c(F)c2n1C1CCC1)c1ccccc1 ; C[C@](O)(CC(=O)Nc1nc2ccc(C#N)c(F)c2n1C1CCC1)c1ccccc1 ; COc1cc2nc(NC(=O)CC(C)(C)O)n(C3CCC3)c2cc1C1CC1 ; CC(C)(O)CC(=O)Nc1nc2ccc(C3CC3)c(F)c2n1C1CCC1 ; C[C@](O)(CC(=O)Nc1nc2c(F)cc(C#N)cc2n1C1CCC1)c1ccccc1]

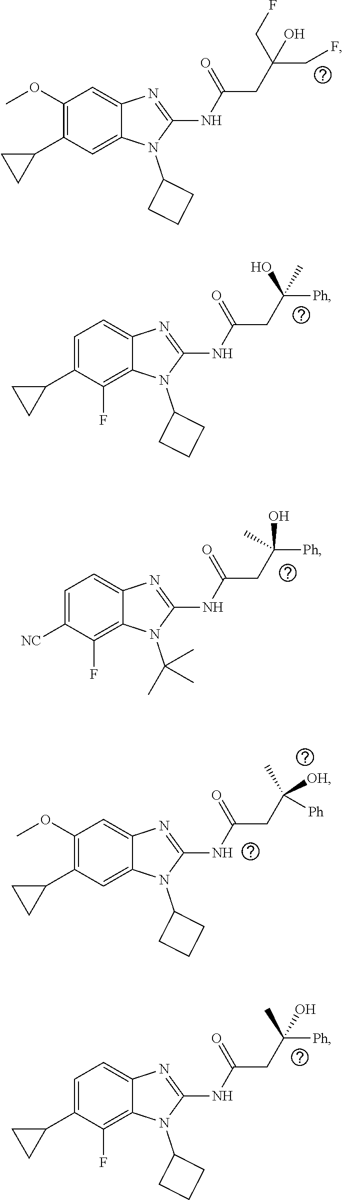 [SMILES: COc1cc2nc(NC(=O)CC(O)(CF)CF)n(C3CCC3)c2cc1C1CC1 ; C[C@@](O)(CC(=O)Nc1nc2ccc(C3CC3)c(F)c2n1C1CCC1)c1ccccc1 ; CC(C)(C)n1c(NC(=O)C[C@@](C)(O)c2ccccc2)nc2ccc(C#N)c(F)c21 ; COc1cc2nc(NC(=O)C[C@@](C)(O)c3ccccc3)n(C3CCC3)c2cc1C1CC1 ; C[C@](O)(CC(=O)Nc1nc2ccc(C3CC3)c(F)c2n1C1CCC1)c1ccccc1]

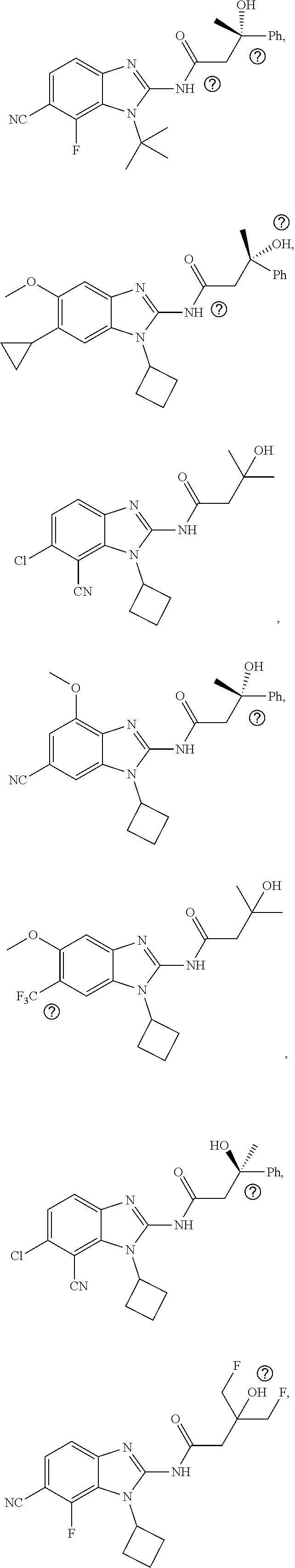

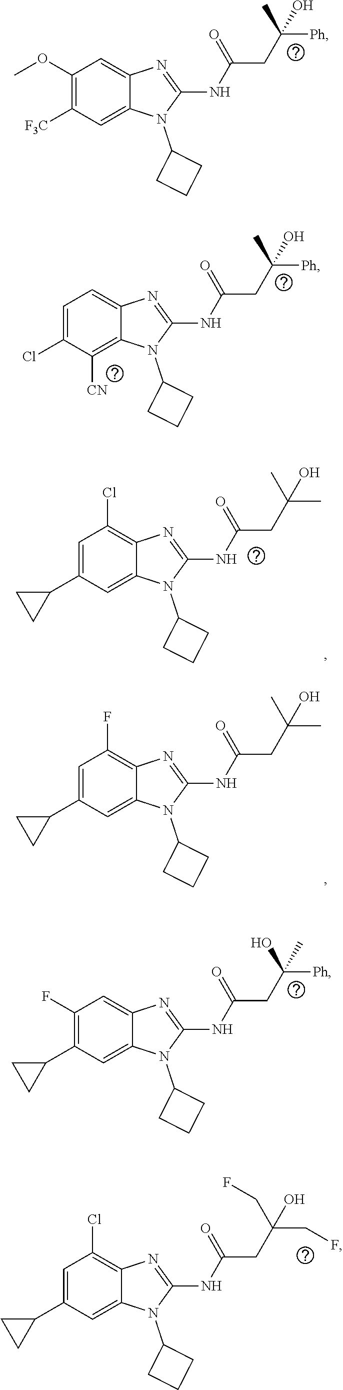 [SMILES: COc1cc2nc(NC(=O)C[C@](C)(O)c3ccccc3)n(C3CCC3)c2cc1C(F)(F)F ; C[C@](O)(CC(=O)Nc1nc2ccc(Cl)c(C#N)c2n1C1CCC1)c1ccccc1 ; CC(C)(O)CC(=O)Nc1nc2c(Cl)cc(C3CC3)cc2n1C1CCC1 ; CC(C)(O)CC(=O)Nc1nc2c(F)cc(C3CC3)cc2n1C1CCC1 ; C[C@@](O)(CC(=O)Nc1nc2cc(F)c(C3CC3)cc2n1C1CCC1)c1ccccc1 ; O=C(CC(O)(CF)CF)Nc1nc2c(Cl)cc(C3CC3)cc2n1C1CCC1]

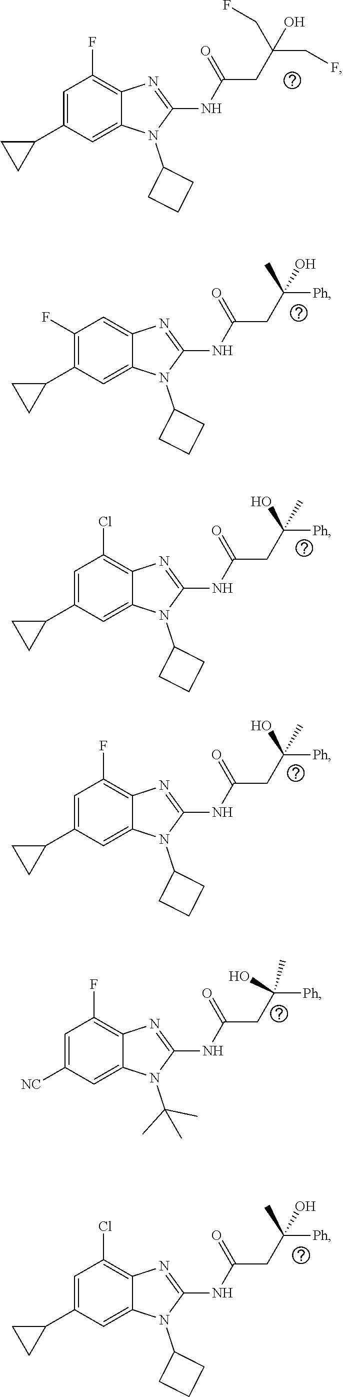 [SMILES: O=C(CC(O)(CF)CF)Nc1nc2c(F)cc(C3CC3)cc2n1C1CCC1 ; C[C@](O)(CC(=O)Nc1nc2cc(F)c(C3CC3)cc2n1C1CCC1)c1ccccc1 ; C[C@@](O)(CC(=O)Nc1nc2c(Cl)cc(C3CC3)cc2n1C1CCC1)c1ccccc1 ; C[C@@](O)(CC(=O)Nc1nc2c(F)cc(C3CC3)cc2n1C1CCC1)c1ccccc1 ; CC(C)(C)n1c(NC(=O)C[C@@](C)(O)c2ccccc2)nc2c(F)cc(C#N)cc21 ; C[C@](O)(CC(=O)Nc1nc2c(Cl)cc(C3CC3)cc2n1C1CCC1)c1ccccc1]

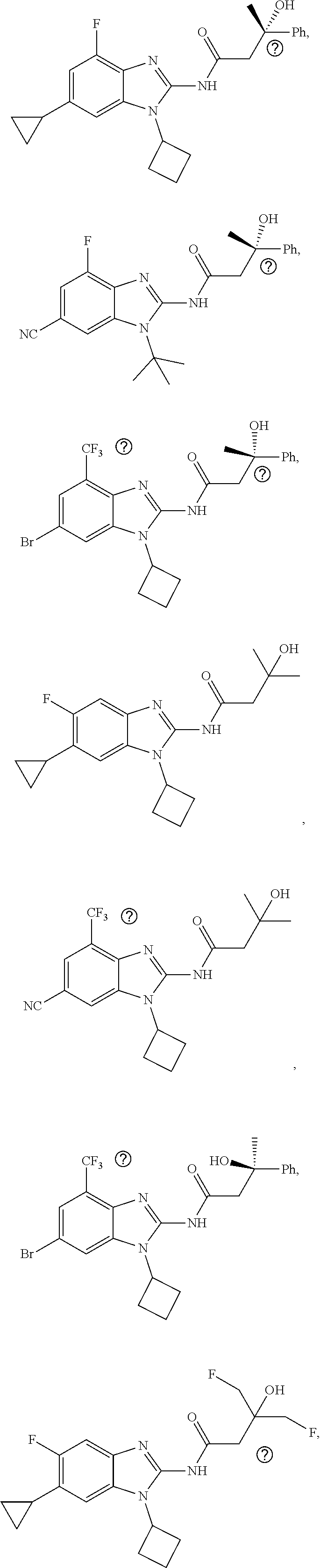

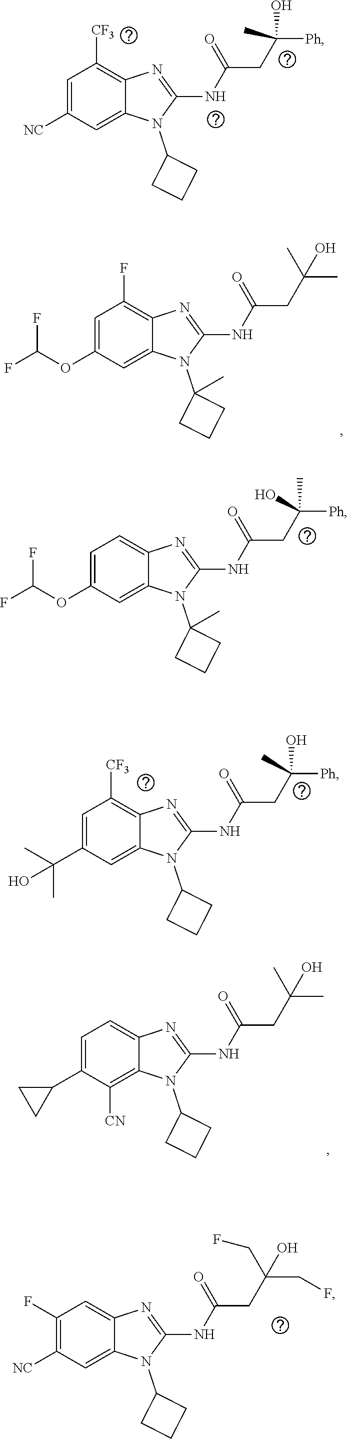

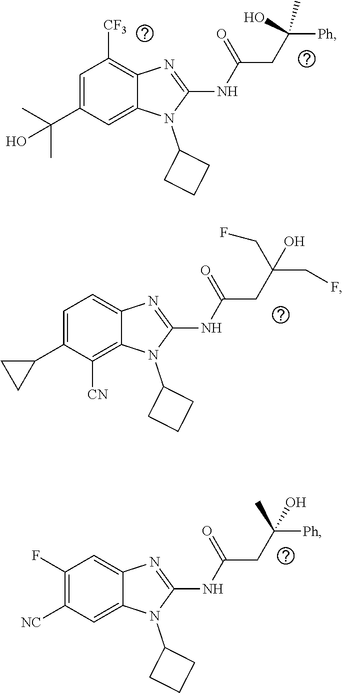 [SMILES: CC(C)(O)c1cc(C(F)(F)F)c2nc(NC(=O)C[C@@](C)(O)c3ccccc3)n(C3CCC3)c2c1 ; N#Cc1c(C2CC2)ccc2nc(NC(=O)CC(O)(CF)CF)n(C3CCC3)c12 ; C[C@](O)(CC(=O)Nc1nc2cc(F)c(C#N)cc2n1C1CCC1)c1ccccc1]

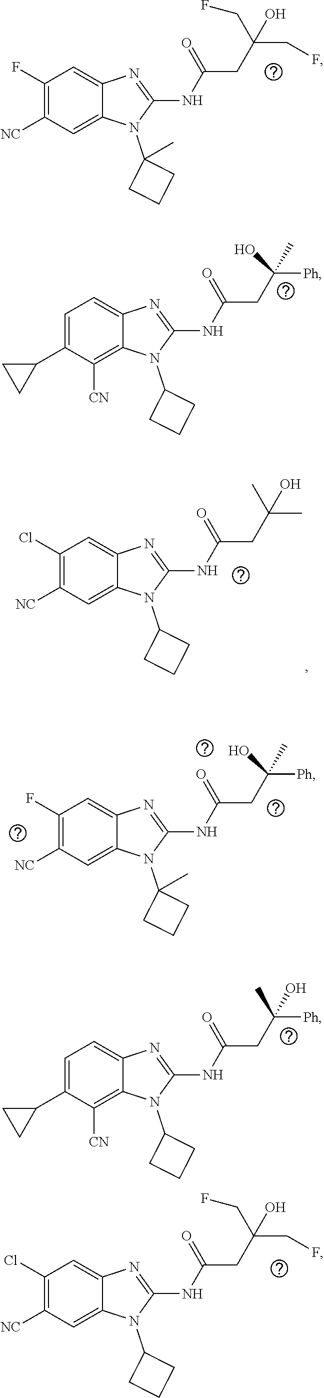

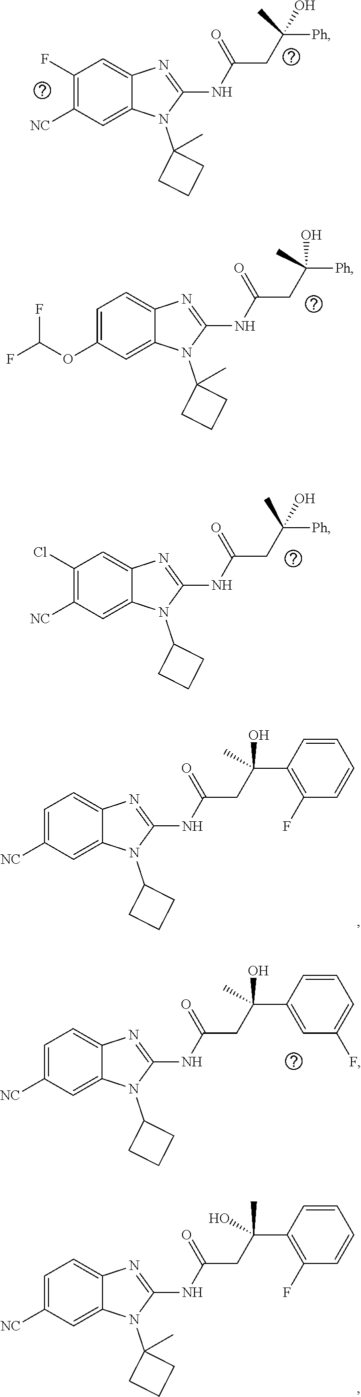 [SMILES: CC1(n2c(NC(=O)C[C@](C)(O)c3ccccc3)nc3cc(F)c(C#N)cc32)CCC1 ; CC1(n2c(NC(=O)C[C@](C)(O)c3ccccc3)nc3ccc(OC(F)F)cc32)CCC1 ; C[C@](O)(CC(=O)Nc1nc2cc(Cl)c(C#N)cc2n1C1CCC1)c1ccccc1 ; C[C@@](O)(CC(=O)Nc1nc2ccc(C#N)cc2n1C1CCC1)c1ccccc1F ; C[C@@](O)(CC(=O)Nc1nc2ccc(C#N)cc2n1C1CCC1)c1cccc(F)c1 ; CC1(n2c(NC(=O)C[C@](C)(O)c3ccccc3F)nc3ccc(C#N)cc32)CCC1]

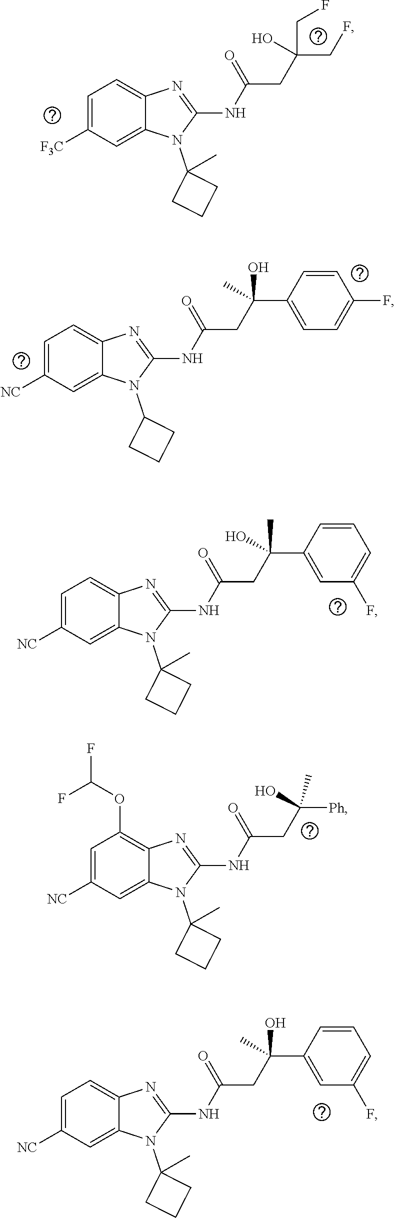 [SMILES: CC1(n2c(NC(=O)CC(O)(CF)CF)nc3ccc(C(F)(F)F)cc32)CCC1 ; C[C@@](O)(CC(=O)Nc1nc2ccc(C#N)cc2n1C1CCC1)c1ccc(F)cc1 ; CC1(n2c(NC(=O)C[C@](C)(O)c3cccc(F)c3)nc3ccc(C#N)cc32)CCC1 ; CC1(n2c(NC(=O)C[C@@](C)(O)c3ccccc3)nc3c(OC(F)F)cc(C#N)cc32)CCC1 ; CC1(n2c(NC(=O)C[C@@](C)(O)c3cccc(F)c3)nc3ccc(C#N)cc32)CCC1]

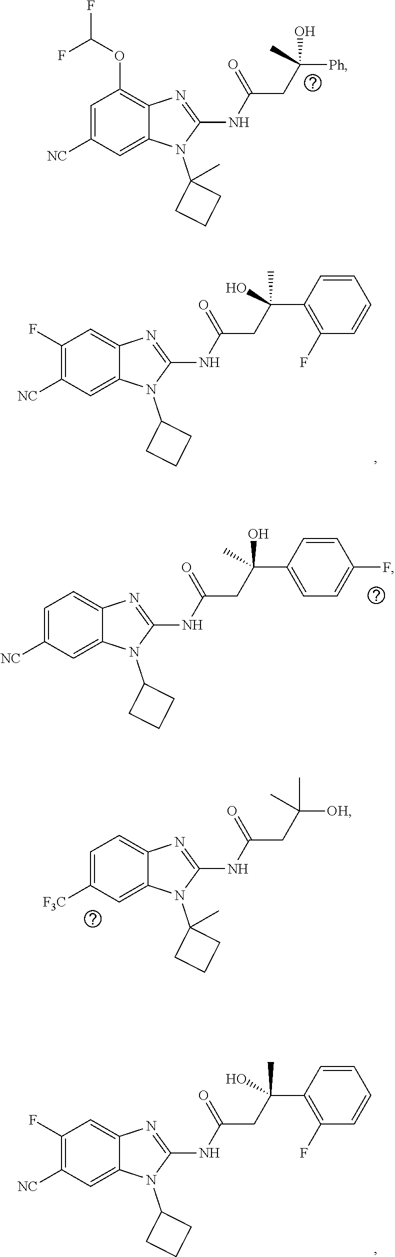 [SMILES: CC1(n2c(NC(=O)C[C@](C)(O)c3ccccc3)nc3c(OC(F)F)cc(C#N)cc32)CCC1 ; C[C@@](O)(CC(=O)Nc1nc2cc(F)c(C#N)cc2n1C1CCC1)c1ccccc1F ; C[C@@](O)(CC(=O)Nc1nc2ccc(C#N)cc2n1C1CCC1)c1ccc(F)cc1 ; CC(C)(O)CC(=O)Nc1nc2ccc(C(F)(F)F)cc2n1C1(C)CCC1 ; C[C@](O)(CC(=O)Nc1nc2cc(F)c(C#N)cc2n1C1CCC1)c1ccccc1F]

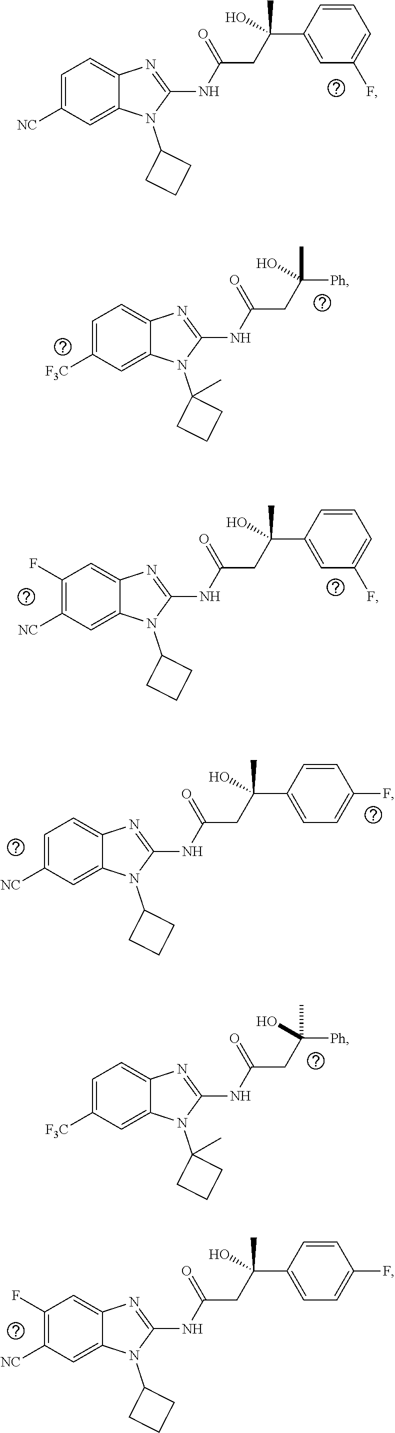 [SMILES: C[C@](O)(CC(=O)Nc1nc2ccc(C#N)cc2n1C1CCC1)c1cccc(F)c1 ; CC1(n2c(NC(=O)C[C@](C)(O)c3ccccc3)nc3ccc(C(F)(F)F)cc32)CCC1 ; C[C@](O)(CC(=O)Nc1nc2cc(F)c(C#N)cc2n1C1CCC1)c1cccc(F)c1 ; C[C@](O)(CC(=O)Nc1nc2ccc(C#N)cc2n1C1CCC1)c1ccc(F)cc1 ; CC1(n2c(NC(=O)C[C@@](C)(O)c3ccccc3)nc3ccc(C(F)(F)F)cc32)CCC1 ; C[C@](O)(CC(=O)Nc1nc2cc(F)c(C#N)cc2n1C1CCC1)c1ccc(F)cc1]

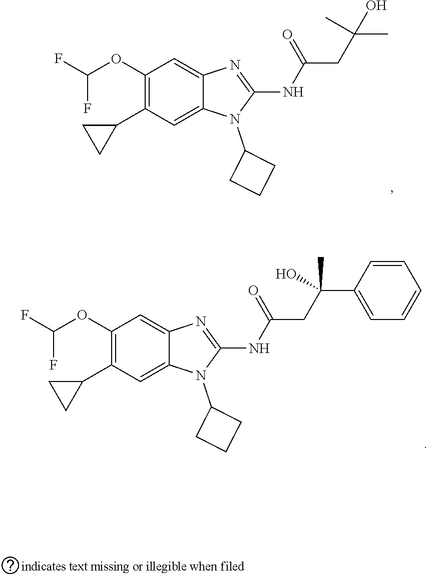 [SMILES: CC(C)(O)CC(=O)Nc1nc2cc(OC(F)F)c(C3CC3)cc2n1C1CCC1 ; C[C@](O)(CC(=O)Nc1nc2cc(OC(F)F)c(C3CC3)cc2n1C1CCC1)c1ccccc1]

### Formula 8

In an embodiment, the Kv7 channel activator may be selected from one of the following compounds according to Formula 8. Such compounds are described in U.S. Pat. No. 9,650,376, published on Feb. 4, 2014 and corresponding to U.S. application Ser. No. 14/776,271 filed Mar. 17, 2014; which is incorporated by reference in its entirety herein. In the case of any conflict of terminology in the context of Formula 8, this reference incorporated by reference herein controls.

In an embodiment, the Kv7 channel activator is a compound according to formula 8:

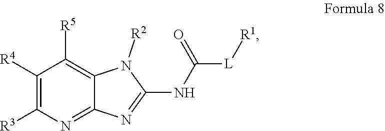

wherein, L is CH₂, CF₂, CHCH₃, CH₂CH₂, C₃H₆, CH₂O, C₂H₄O, or C₃H₆O with the O of CH₂O, C₂H₄O, or C₃H₆O bonded with R¹; wherein R¹ is optionally substituted C₁₋₂ alkyl, optionally substituted C₅-10 cycloalkyl, optionally substituted C₁₋₁₂-O-alkyl, optionally substituted C₆-10 aryl, optionally substituted C₆-10-O-aryl, or optionally substituted C₂-9 heterocyclyl, wherein the optional substituents of R¹ are independently R^{A}, F, Cl, CN, OR^{A}, CF₃, NR^{A}R^{B}, COR^{A}, CO₂R^{A}, OCOR^{A}, NR^{A}COR^{B}, CONR^{A}R^{B}, wherein R^{A} and R^{B} are independently H or C₁₋₁₂ alkyl; wherein R² is optionally substituted C₂₋₄ acyclic alkyl, optionally substituted cyclobutyl, optionally substituted C₆-10 aryl, or optionally substituted C₂₋₉ heterocyclyl, wherein the optional substituents of R² are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ alkylthio, or C₁₋₆ alkylsulfonyl; wherein R³, R⁴, and R⁵ are independently H, F, Cl, Br, I, CN, optionally substituted C₁₋₁₂ alkyl, optionally substituted C₁₋₁₂-O-alkyl, optionally substituted C₂-9 heterocyclyl, optionally substituted C₆-10 aryl, optionally substituted C₂₋₉-O-heterocyclyl, optionally substituted C₆-10 O-aryl, optionally substituted C₁₋₁₂ acylamino, optionally substituted C₁₋₁₂ aminoacyl, or optionally substituted C₁₋₁₂ aminoalkyl, wherein the optional substituents of R³, R⁴, or R⁵ are independently F, Cl, Br, I, CN, C₁₋₆ alkyl, C₁₋₆-O-alkyl, C₁₋₆ alkylamine, C₁₋₆ aminoalkyl, C₁₋₆ aminoacyl, C₁₋₆ acylamino, C₁₋₆ alkylthio, or C₁₋₆ alkylsulfonyl.

In further embodiments, R² is optionally substituted C₄H₉, optionally substituted cyclobutyl, optionally substituted C₆-10 aryl, or optionally substituted C₂₋₉ heterocyclyl.

In further embodiments, R² is optionally substituted C₄H₉, or optionally substituted cyclobutyl.

In further embodiments, R² is optionally substituted phenyl, or optionally substituted C₂₋₉ heterocyclyl.

In further embodiments, R¹ is optionally substituted phenyl.

In further embodiments, R¹ is optionally substituted phenyl.

In further embodiments, R¹ is optionally substituted cyclopentyl or optionally substituted cyclohexyl.

In further embodiments, R¹ is C₂₋₄-O-alkyl.

In further embodiments, R¹ is optionally substituted tetrahydrofuranyl or optionally substituted tetrahydro-2H-pyranyl.

In further embodiments, R¹ is selected from the group consisting of:

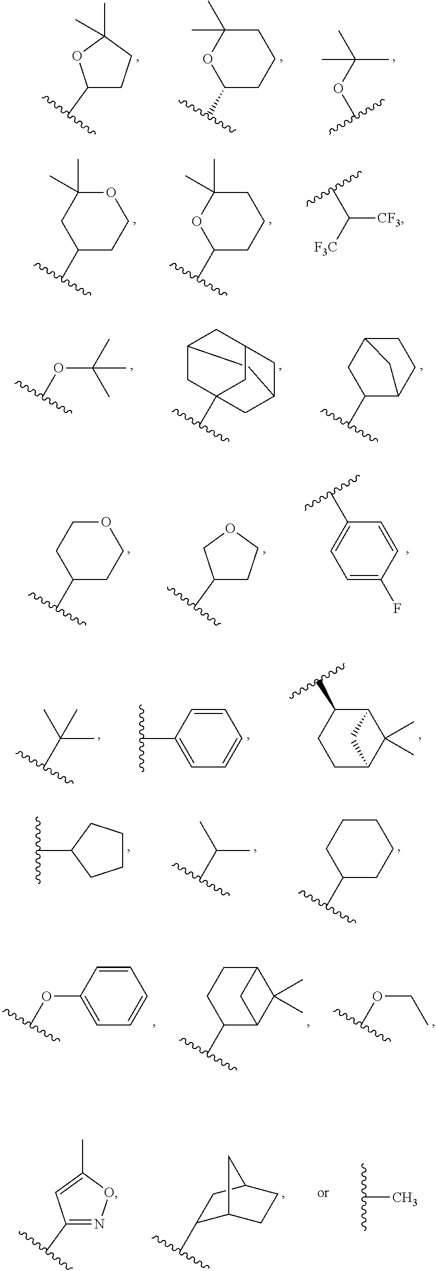

In further embodiments, R² is selected from the group consisting of:

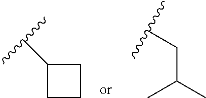

In further embodiments, R³ is CF₃, Cl, CN, OCH₃, or H.

In further embodiments, R¹ is selected from the group consisting of:

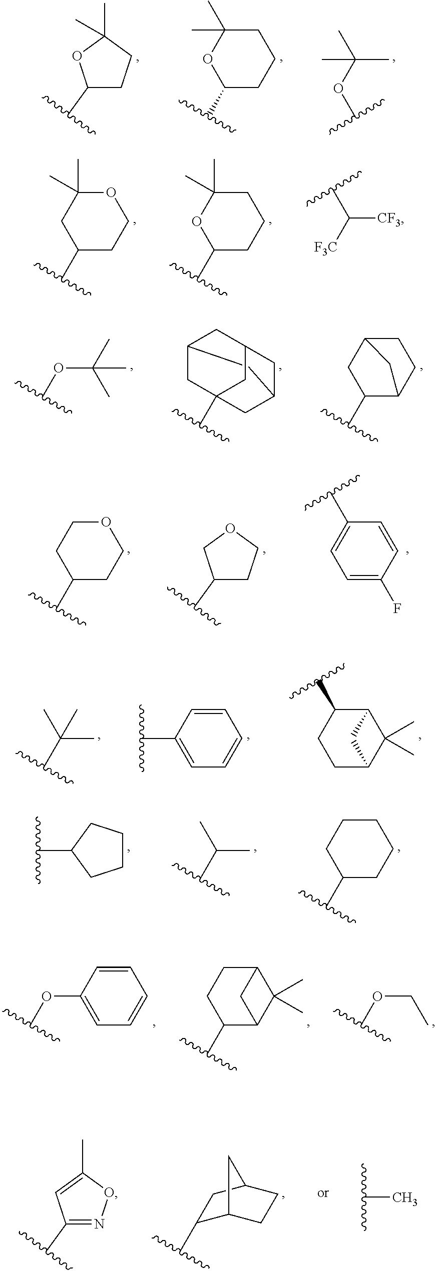

In further embodiments, R⁴ is CH₃, CF₃, Cl, or H.

In further embodiments, R² is selected from the group consisting of:

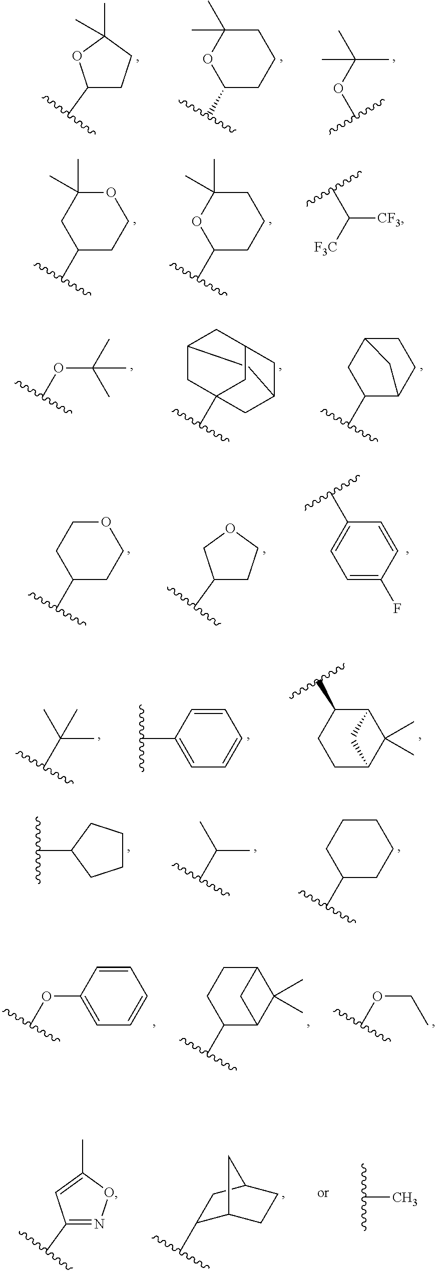

### Formula 9

In an embodiment, the Kv7 channel activator may be selected from one of the following compounds according to Formula 9. Such compounds are described in International Publication No. WO2021023616A1, published Feb. 11, 2021 and corresponding to International Application No. PCT/EP2020/071514 filed Jul. 30, 2020; International Publication No. WO2019161877A1, published Aug. 29, 2019 and corresponding to International Application No. PCT/EP2018/054057 filed Feb. 20, 2018; US Publication No. US20220280455A1, published Sep. 8, 2022 and corresponding to U.S. application Ser. No. 17/631,762 filed Jul. 30, 2020; US Publication No. US20210032196A1, published Feb. 4, 2021 and corresponding to U.S. application Ser. No. 16/943,872 filed Jul. 30, 2020; US Publication No. US20210032196A1, published Feb. 4, 2021 and corresponding to U.S. application Ser. No. 16/943,872 filed Jul. 30, 2020; which are incorporated by reference in their entirety herein. In the case of any conflict of terminology in the context of Formula 9, these references incorporated by reference herein control.

In an embodiment, the Kv7 channel activator is a compound according to formula 9:

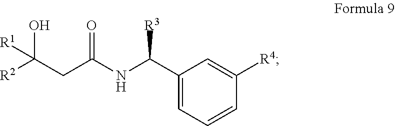

wherein, R1 is selected from the group consisting of C₁-C₆ alkyl, CF₃, CH₂CF₃, CF₂CHF₂, C₃-C₈ cycloalkyl, wherein said C₃-C₈ cycloalkyl may be substituted with 1 or 2 substituents selected from the group consisting of C₁-C₃ alkyl, F, CHF₂ and CF₃; and R2 is H, C₁-C₆ alkyl or CF₃; or R1 and R2 combine to form C₃-C₅ cycloalkyl optionally substituted with 1 or 2 F, CHF₂ or CF₃; and R3 is C₁-C₃ alkyl or CH₂O—C₁₋₃ alkyl, said C₁-C₃ alkyl or CH₂O—C₁-C₃ is alkyl substituted with C≡N, 3 F or C₃-C₅ cycloalkyl; R4 is selected from the group consisting of OCF₃, or OCHF₂

In further embodiments, R4 is OCF₃ or OCHF₂.

In further embodiments, R3 is selected from the group consisting of CH₂—O—CF₃, CH₂—O-cyclopropyl, CH₂—C≡N.

In further embodiments R1 is C₃-C₄ cycloalkyl optionally substituted with 1 or 2 C₁-C₃ alkyl, F, CHF₂ or CF₃.

In further embodiments, R1 and R2 combine to form cyclobutyl optionally substituted with 1 or 2 F and R4 is OCF₃ or OCHF₂.

In further embodiments, the Kv7 channel activator is selected from the group consisting of: (S)—N—((R)-2-cyclopropoxy-1-(3-(difluoromethoxy)phenyl)ethyl)-3-hydroxy-4,4-dimethylpentanamide; (S)—N—((R)-1-(3-(difluoromethoxy)phenyl)-2-(trifluoromethoxy)ethyl)-3-hydroxy-4,4-dimethylpentanamide; (S)—N—((R)-1-(3-(trifluoromethoxy)phenyl)-2-(trifluoromethoxy)ethyl)-3-hydroxy-4,4-dimethylpentanamide; (S)—N—((S)-2-cyano-1-(3-(trifluoromethoxy)phenyl)ethyl)-3-hydroxy-4,4-dimethylpentanamide; (S)—N—((S)-3-cyano-1-(3-(trifluoromethoxy)phenyl)propyl)-3-hydroxy-4,4-dimethylpentanamide; (R)—N-(2-cyclopropoxy-1-(3-(trifluoromethoxy)phenyl) ethyl)-2-(3,3-difluoro-1-hydroxycyclobutyl) acetamide; (R)—N-(2-cyclopropoxy-1-(3-(difluoromethoxy)phenyl)ethyl)-2-(3,3-difluoro-1-hydroxycyclobutyl) acetamide; (R)-2-(3,3-difluoro-1-hydroxycyclobutyl)-N-(1-(3-(difluoromethoxy)phenyl)-2-(trifluoromethoxy)ethyl) acetamide; and(S)—N-(2-cyano-1-(3-(trifluoromethoxy)phenyl) ethyl)-2-(3,3-difluoro-1-hydroxycyclobutyl) acetamide or a pharmaceutically acceptable salt of any of these compounds.

In further embodiments, the Kv7 channel activator is selected from the group consisting of (R)—N—((R)-2-(difluoromethoxy)-1-(3-(difluoromethoxy)phenyl)ethyl)-3-hydroxy-4,4-dimethylpentanamide; (S)—N—((R)-2-(difluoromethoxy)-1-(3-(difluoromethoxy)phenyl)ethyl)-3-hydroxy-4,4-dimethylpentanamide; (S)-3-hydroxy-4,4-dimethyl-N—((S)-1-(3-(2,2,2-trifluoroethoxy)phenyl)ethyl)pentanamide; (R)-3-hydroxy-4,4-dimethyl-N—((S)-1-(3-(2,2,2-trifluoroethoxy)phenyl)ethyl)pentanamide; (R)—N—((R)-2-(difluoromethoxy)-1-(3-(difluoromethoxy)phenyl)ethyl)-3-hydroxy-3-(1-(trifluoro-methyl)cyclopropyl) propanamide; (S)—N—((R)-2-(difluoromethoxy)-1-(3-(difluoromethoxy)phenyl)ethyl)-3-hydroxy-3-(1-(trifluoro-methyl)cyclopropyl)propanamide; (R)—N—((R)-2-(difluoromethoxy)-1-(3-(trifluoromethoxy)phenyl)ethyl)-3-hydroxy-3-(1-(trifluoromethyl)cyclopropyl)propanamide; (S)—N—((R)-2-(difluoromethoxy)-1-(3-(trifluoromethoxy)phenyl)ethyl)-3-hydroxy-3-(1-(trifluoromethyl)cyclopropyl)propanamide; (R)-3-(3,3-difluorocyclobutyl)-N—((R)-2-(difluoromethoxy)-1-(3-(difluoromethoxy)phenyl)ethyl)-3-hydroxypropanamide; (S)-3-(3,3-difluorocyclobutyl)-N—((R)-2-(difluoromethoxy)-1-(3-(difluoromethoxy)phenyl)ethyl)-3-hydroxypropanamide; (R)-3-(3,3-difluorocyclobutyl)-N—((R)-2-(difluoromethoxy)-1-(3-(trifluoromethoxy)phenyl) ethyl)-3-hydroxypropanamide; (S)-3-(3,3-difluorocyclobutyl)-N—((R)-2-(difluoromethoxy)-1-(3-(trifluoromethoxy)phenyl)ethyl)-3-hydroxypropanamide; (S)-3-(3,3-difluorocyclobutyl)-N—((S)-1-(3-(difluoromethoxy)phenyl)butyl)-3-hydroxypropanamide; (R)-3-(3,3-difluorocyclobutyl)-N—((S)-1-(3-(difluoromethoxy)phenyl)butyl)-3-hydroxypropanamide; (S)—N—((R)-2-(difluoromethoxy)-1-(3-(difluoromethoxy)phenyl)ethyl)-3-(1-ethylcyclopropyl)-3-hydroxypropanamide; (R)—N—((R)-2-(difluoromethoxy)-1-(3-(difluoromethoxy)phenyl)ethyl)-3-(1-ethylcyclopropyl)-3-hydroxypropanamide; (S)—N—((S)-1-(3-(difluoromethoxy)phenyl)butyl)-3-hydroxy-4,4-dimethylpentanamide; (S)—N—((S)-1-(3-(difluoromethoxy)phenyl)-4,4-difluorobutyl)-3-hydroxy-4,4-dimethylpentanamide; (S)—N—((S)-1-(3-(difluoromethoxy)phenyl)-3,3-difluoropropyl)-3-hydroxy-4,4-dimethylpentanamide; (S)—N—((S)-1-(3-(difluoromethoxy)phenyl)ethyl)-3-hydroxy-4,4-dimethylpentanamide; (R)-2-(3,3-difluoro-1-hydroxycyclobutyl)-N-(2-(difluoromethoxy)-1-(3-(difluoromethoxy)phenyl)ethyl) acetamide; (R)-2-(3,3-difluoro-1-hydroxycyclobutyl)-N-(2-(difluoromethoxy)-1-(3-(trifluoromethoxy)phenyl)ethyl) acetamide; (S)-2-(3,3-difluoro-1-hydroxycyclobutyl)-N-(1-(3-(difluoromethoxy)phenyl)butyl) acetamide; (S)-2-(3,3-difluoro-1-hydroxycyclobutyl)-N-(1-(3-(difluoromethoxy)phenyl)-4,4-difluorobutyl) acetamide; (S)-2-(3,3-difluoro-1-hydroxycyclobutyl)-N-(1-(3-(trifluoromethoxy)phenyl)propyl) acetamide; (S)—N-(3,3-difluoro-1-(3-(trifluoromethoxy)phenyl)propyl)-2-(3,3-difluoro-1-hydroxycyclobutyl) acetamide; (S)—N—((R)-2-(difluoromethoxy)-1-(3-(difluoromethoxy)phenyl)ethyl)-3-hydroxy-4,4-dimethylpentanamide; (R)—N—((R)-2-(difluoromethoxy)-1-(3-(difluoromethoxy)phenyl)ethyl)-3-(1-fluorocyclopropyl)-3-hydroxybutanamide; (S)—N—((R)-2-(difluoromethoxy)-1-(3-(difluoromethoxy)phenyl) ethyl)-3-(1-fluorocyclopropyl)-3-hydroxybutanamide; (S)—N—((R)-2-(difluoromethoxy)-1-(3-(trifluoromethoxy)phenyl)ethyl)-3-(1-fluorocyclopropyl)-3-hydroxybutanamide; (R)—N—((R)-2-(difluoromethoxy)-1-(3-(trifluoromethoxy)phenyl)ethyl)-3-(1-fluorocyclopropyl)-3-hydroxybutanamide; (R)-3-cyclopropyl-N—((R)-2-(difluoromethoxy)-1-(3-(trifluoromethoxy)phenyl)ethyl)-3-hydroxybutanamide; (S)-3-cyclopropyl-N—((R)-2-(difluoromethoxy)-1-(3-(trifluoromethoxy)phenyl)ethyl)-3-hydroxybutanamide; (R)—N—((R)-2-(difluoromethoxy)-1-(3-(difluoromethoxy)phenyl)ethyl)-5,5,5-trifluoro-3-hydroxy-3-methylpentanamide; (S)—N—((R)-2-(difluoromethoxy)-1-(3-(difluoromethoxy)phenyl)ethyl)-5,5,5-trifluoro-3-hydroxy-3-methylpentanamide; (R)—N—((R)-2-(difluoromethoxy)-1-(3-(difluoromethoxy)phenyl)ethyl)-3-hydroxy-3,5-dimethylhexanamide; (S)—N—((R)-2-(difluoromethoxy)-1-(3-(difluoromethoxy)phenyl)ethyl)-3-hydroxy-3,5-dimethylhexanamide; (S)—N—((R)-2-(difluoromethoxy)-1-(3-(difluoromethoxy)phenyl)ethyl)-3-hydroxy-3,4-dimethylpentanamide; (R)—N—((R)-2-(difluoromethoxy)-1-(3-(difluoromethoxy)phenyl)ethyl)-3-hydroxy-3,4-dimethylpentanamide; (S)—N—((R)-2-(difluoromethoxy)-1-(3-(difluoromethoxy)phenyl)ethyl)-3-(3,3-dimethylcyclobutyl)-3-hydroxypropanamide; (R)—N—((R)-2-(difluoromethoxy)-1-(3-(difluoromethoxy)phenyl)ethyl)-3-(3,3-dimethylcyclobutyl)-3-hydroxypropanamide; (S)-3-cyclopentyl-N—((R)-2-(difluoromethoxy)-1-(3-(difluoromethoxy)phenyl)ethyl)-3-hydroxypropanamide; (R)-3-cyclopentyl-N—((R)-2-(difluoromethoxy)-1-(3-(difluoromethoxy)phenyl)ethyl)-3-hydroxypropanamide; (R)-3-(1-fluorocyclopropyl)-3-hydroxy-N—((R)-2-methoxy-1-(3-(trifluoromethoxy)phenyl)ethyl) butanamide; and(S)-3-(1-fluorocyclopropyl)-3-hydroxy-N—((R)-2-methoxy-1-(3-(trifluoromethoxy)phenyl)ethyl) butanamide; or a pharmaceutically acceptable salt of any of these compounds.

In further embodiments, the Kv7 channel activator is a compound according to formula 8 wherein R¹ is selected from the group consisting of C₁-C₆ alkyl, CF₃, CH₂CF₃, CF₂CHF₂, C₃-C₈ cycloalkyl, wherein said C₃-C₈ cycloalkyl may be substituted with 1 or 2 F, CHF₂ or CF₃, and R² is H, C₁-C₆ alkyl or CF₃; or R¹ and R² combine to form C₃-C₅ cycloalkyl optionally substituted with 1 or 2 F, CHF₂ or CF₃; R³ is C₁-C₃ alkyl or CH₂O—C₁₋₃ alkyl, said C₁-C₃ alkyl or CH₂O—C₁₋₃ alkyl may optionally be substituted with 1 or 2 F; and R⁴ is selected from the group consisting of OCF₃, OCH₂CF₃ or OCHF₂.

In further embodiments, R⁴ is OCF₃ or OCHF₂.

In further embodiments, R² is H or CH₃.

In further embodiments, R³ is CH₂O—C₁₋₃ alkyl.

In further embodiments, R¹ is C₃-C₄ cycloalkyl optionally substituted with 1 or 2 F, CHF₂ or CF₃.

In further embodiments, R¹ is t-butyl and R² is H and R⁴ is OCF₃, OCH₂CF₃, OCHF₂ or CF₃.

In further embodiments, R¹ and R² combine to form cyclobutyl optionally substituted with 1 or 2 F and R⁴ is OCF₃, OCH₂CF₃, OCHF₂ or CF₃.

In further embodiments, the Kv7 channel activator is selected from the group consisting of: (S)-3-hydroxy-4,4-dimethyl-N-[(1S)-1-[3-(trifluoromethoxy)phenyl]ethyl]pentanamide, (R)-3-hydroxy-4,4-dimethyl-N-[(1S)-1-[3-(trifluoromethoxy)phenyl]ethyl]pentanamide, (S)-3-hydroxy-4,4-dimethyl-N—((S)-1-(3-(2,2,2-trifluoroethoxy)phenyl)ethyl)pentanamide, (R)-3-hydroxy-4,4-dimethyl-N—((S)-1-(3-(2,2,2-trifluoroethoxy)phenyl)ethyl)pentanamide, (S)—N—((S)-1-(3-(difluoromethoxy)phenyl)ethyl)-3-hydroxy-4,4-dimethylpentanamide, (R)—N—((S)-1-(3-(difluoromethoxy)phenyl)ethyl)-3-hydroxy-4,4-dimethylpentanamide, (S)-3-hydroxy-4,4-dimethyl-N—((S)-1-(3-(trifluoromethyl)phenyl)ethyl)pentanamide (R)-3-hydroxy-4,4-dimethyl-N—((S)-1-(3-(trifluoromethyl)phenyl)ethyl)pentanamide, (S)-3-hydroxy-4,4-dimethyl-N—((S)-1-(3-(trifluoromethoxy)phenyl)propyl)pentanamide, (R)-3-hydroxy-4,4-dimethyl-N—((S)-1-(3-(trifluoromethoxy)phenyl)propyl)pentanamide, (S)-3-(3,3-difluorocyclobutyl)-3-hydroxy-N—((S)-1-(3-(trifluoromethoxy)phenyl)ethyl)propanamide, (R)-3-(3,3-difluorocyclobutyl)-3-hydroxy-N—((S)-1-(3-(trifluoromethoxy)phenyl)ethyl)propanamide, (S)-3-hydroxy-4-methyl-N—((S)-1-(3-(2,2,2-trifluoroethoxy)phenyl)ethyl)pentanamide, (R)-3-hydroxy-4-methyl-N—((S)-1-(3-(2,2,2-trifluoroethoxy)phenyl)ethyl)pentanamide, (S)-3-(1-(difluoromethyl)cyclopropyl)-3-hydroxy-N—((S)-1-(3-(trifluoro-methoxy)phenyl)ethyl)propanamide, (R)-3-(1-(difluoromethyl)cyclopropyl)-3-hydroxy-N—((S)-1-(3-(trifluoromethoxy)phenyl)ethyl)propanamide, (R)-3-hydroxy-N—((S)-1-(3-(trifluoromethoxy)phenyl)ethyl)-3-(1-(trifluoromethyl)cyclopropyl)propanamide, (S)-3-hydroxy-N—((S)-1-(3-(trifluoromethoxy)phenyl)ethyl)-3-(1-(trifluoromethyl)cyclopropyl)propanamide, (S)-3-hydroxy-4-methyl-N—((S)-1-(3-(trifluoromethoxy)phenyl)ethyl)pentanamide, (R)-3-hydroxy-4-methyl-N—((S)-1-(3-(trifluoromethoxy)phenyl)ethyl)pentanamide, N—((R)-2-(difluoromethoxy)-1-(3-(trifluoromethoxy)phenyl)ethyl)-3-(R)-hydroxy-4,4-dimethylpentanamide, N—((R)-2-(difluoromethoxy)-1-(3-(trifluoromethoxy)phenyl)ethyl)-3-(S)-hydroxy-4,4-dimethylpentanamide, (S)-3-hydroxy-N—((R)-2-methoxy-1-(3-(trifluoromethoxy)phenyl)ethyl)-4,4-dimethylpentanamide, (R)-3-hydroxy-N—((R)-2-methoxy-1-(3-(trifluoromethoxy)phenyl)ethyl)-4,4-dimethylpentanamide, (S)-2-(3,3-difluoro-1-hydroxycyclobutyl)-N-(1-(3-(trifluoromethoxy)phenyl)ethyl) acetamide, (S)-2-(1-hydroxycyclobutyl)-N-(1-(3-(2,2,2-trifluoroethoxy)phenyl)ethyl) acetamide, (3R)-3-hydroxy-4-methyl-N-[(1S)-1-[3-(2,2,2-trifluoroethoxy)phenyl]ethyl]-3-(trifluoromethyl)pentanamide, (3S)-3-hydroxy-4-methyl-N-[(1S)-1-[3-(2,2,2-trifluoroethoxy)phenyl]ethyl]-3-(trifluoromethyl)pentanamide, 4,4,4-Trifluoro-3-hydroxy-N-[(1S)-1-[3-(trifluoromethoxy)phenyl]ethyl]-3-(trifluoromethyl) butanamide, (R)-4,4,5,5-tetrafluoro-3-hydroxy-3-methyl-N—((S)-1-(3-(trifluoromethoxy)phenyl)ethyl)pentanamide, (S)-4,4,5,5-tetrafluoro-3-hydroxy-3-methyl-N—((S)-1-(3-(trifluoromethoxy)phenyl)ethyl)pentanamide, (R)-5,5,5-trifluoro-3-hydroxy-3-methyl-N—((S)-1-(3-(trifluoromethoxy)phenyl)ethyl)pentanamide, (S)-5,5,5-trifluoro-3-hydroxy-3-methyl-N—((S)-1-(3-(trifluoro methoxy)phenyl)ethyl)pentanamide, (R)-3-(1-fluorocyclopropyl)-3-hydroxy-N—((S)-1-(3-(trifluoromethoxy)phenyl)ethyl) butanamide, (S)-3-(1-fluorocyclopropyl)-3-hydroxy-N—((S)-1-(3-(trifluoro-methoxy)phenyl)ethyl) butanamide, (R)-2-(1-hydroxycyclopentyl)-N-(2-methoxy-1-(3-(trifluoromethoxy)phenyl)ethyl) acetamide, (R)-3-cyclopropyl-3-hydroxy-N—((S)-1-(3-(2,2,2-trifluoroethoxy)phenyl)ethyl) butanamide, (S)-3-cyclopropyl-3-hydroxy-N—((S)-1-(3-(2,2,2-trifluoroethoxy)phenyl)ethyl) butanamide, (R)-4,4,4-trifluoro-3-hydroxy-3-methyl-N—((S)-1-(3-(2,2,2-trifluoroethoxy)phenyl)ethyl) butanamide, and(S)-4,4,4-trifluoro-3-hydroxy-3-methyl-N—((S)-1-(3-(2,2,2-trifluoroethoxy)phenyl)ethyl) butanamide or a pharmaceutically acceptable salt of any of these compounds.

### Formula 10

In an embodiment, the Kv7 channel activator may be selected from one of the following compounds according to Formula 10. Such compounds are described in International Publication No. WO2009015667A1, published Feb. 5, 2009, and corresponding to International Application No. PCT/DK2008/050191 filed Jul. 31, 2008; International Publication No. WO2004058739A1, published Jul. 15, 2004 and corresponding to International Application No. PCT/DK2003/000906 filed Dec. 18, 2003; U.S. Pat. No. 7,368,472 issued May 6, 2008 and corresponding to U.S. application Ser. No. 10/540,075 filed Dec. 18, 2003; US Publication No. US20100256145A1, published Oct. 7, 2010 and corresponding to U.S. application Ser. No. 12/671,505 filed Jul. 31, 2008; which are incorporated by reference in their entirety herein. In the case of any conflict of terminology in the context of Formula 8, these references incorporated by reference herein control.

In an embodiment, the Kv7 channel activator is a compound according to formula 10:

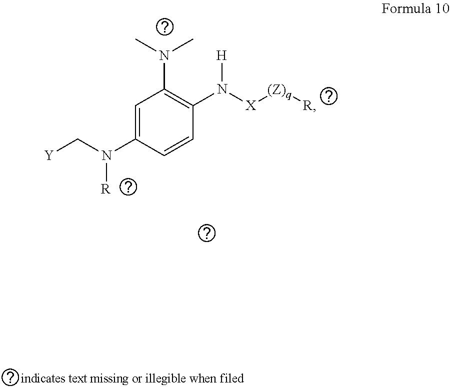

wherein R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl and hydroxy-C₃₋₈-Cycloalk(en)yl; R² and R^{2′} are independently selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, hydroxy-C₁₋₆-alk(en/yn)yl and hydroxy-C₃₋₈-cycloalk(en)yl; R³ is selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₃₋₆-alk(en/yn)yl, hydroxy-C₁₋₆-alk(en/yn)yl, aryl-C₃₋₈-cycloalk(en)yl, NR¹⁰R^{10′}-C₁₋₄-alk(en/yn)yl, NR¹⁰R^{10′}-C₃₋₈-Cycloalk(en)yl and hydroxy-C₃₋₈-cycloalk(en)yl; wherein: R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹⁰ and R^{10′} together with the nitrogen atom to which they are attached form a 4-8 membered saturated or unsaturated ring that optionally contains 1, 2 or 3 further heteroatoms; X is CO or SO₂; Z is O or NR⁴, wherein: R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl; or R³ and R⁴ together with the nitrogen atom to which they are attached form a 4-8 membered saturated or unsaturated ring that optionally contains 1, 2 or 3 further heteroatoms, and the ring formed by R³ and R⁴ and the nitrogen atom is optionally substituted with one or more substituents independently selected from C₁₋₆-alk(en/yn)yl, aryl and aryl-C₁₋₆-alk(en/yn)yl; q is 0 or 1; and Y represents a heteroaryl of one of the following formulas:

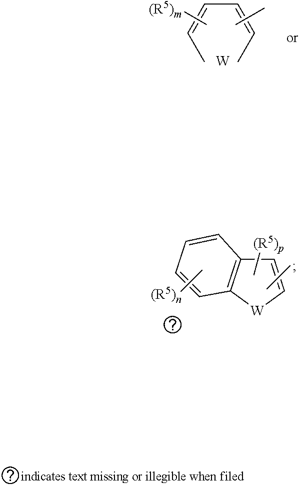

wherein: W is O or S; m is 0, 1, 2 or 3; n is 0, 1, 2, 3 or 4; p is 0 or 1; and each R⁵ is independently selected from the group consisting of C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, halogen, halo-C₁₋₆-alk(en/yn)yl, alk(en/yn)yloxy, —CO—NR⁶R^{6′}, cyano, nitro, —NR⁷R^{4′}, —S—R⁸, —SO₂R⁸, and SO₂OR⁸; wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and aryl; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, aryl and acyl; and R⁸ is selected from the group consisting of C₁₋₆-alk(en/yn)yl, C₁₋₆-cycloalk(en)yl, C₃₋₈-cycloalk(en)yl-C₁₋₆-alk(en/yn)yl, aryl and NR⁹R^{9′}; wherein: R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; or a pharmaceutically acceptable salt thereof.

In further embodiments, the Kv7 channel activator is selected from the group consisting of: {2-Amino-4-[(5-chloro-thiophen-2-ylmethyl)-methyl-amino]-phenyl}-carbamic acid ethyl ester; {2-Amino-4-[(5-chloro-thiophen-2-ylmethyl)-amino]-phenyl}-carbamic acid ethyl ester; {2-Amino-4-[(5-methyl-thiophen-2-ylmethyl)-methyl-amino]-phenyl}-carbamic acid ethyl ester; {2-Amino-4-[(5-bromo-thiophen-2-ylmethyl)-amino]-phenyl}-carbamic acid ethyl ester; {2-Amino-4-[(6-chloro-3-methoxy-benzo[b]thiophen-2-ylmethyl)-amino]-phenyl}-carbamic acid ethyl ester; {(2-Amino-4-[(benzo[b]thiophen-2-ylmethyl)-amino]-phenyl}-carbamic acid ethyl ester; {2-Amino-4-[(5-methyl-thiophen-2-ylmethyl)-amino]-phenyl}-carbamic acid ethyl ester; {2-Amino-4-[(4-bromo-3-methoxy-thiophen-2-ylmethyl)-amino]-phenyl}-carbamic acid ethyl ester; {2-Amino-4-[(5-phenyl-thiophen-2-ylmethyl)-amino]-phenyl}-carbamic acid ethyl ester; {2-Amino-4-[(3-chloro-thiophen-2-ylmethyl-amino]-phenyl}-carbamic acid ethyl ester; (2-Amino-4-{[4-(4-chloro-benzenesulfonyl)-3-methyl-thiophen-2-ylmethyl]-amino}-phenyl)-carbamic acid ethyl ester; {2-Amino-4-[(3-methyl-thiophen-2-ylmethyl)-amino]-phenyl}-carbamic acid ethyl ester; {2-Amino-4-[(5-fluoro-benzofuran-3-ylmethyl)-amino]phenyl}-carbamic acid ethyl ester; {2-Amino-4-[(thiophen-2-ylmethyl)-amino]-phenyl}-carbamic acid ethyl ester; {2-Amino-4-[(4-bromo-thiophen-2-ylmethyl)-amino]-phenyl}-carbamic acid ethyl ester; {2-Amino-4-[(5-ethyl-thiophen-2-ylmethyl)-amino]-phenyl}-carbamic acid ethyl ester; {2-Amino-4-[(thiophen-3-ylmethyl)-amino]-phenyl}-carbamic acid ethyl ester; {2-Amino-4-[(5-chloro-thiophen-2-ylmethyl)-ethyl-amino]-phenyl}-carbamic acid ethyl ester; {2-Amino-4-[(benzo[b]thiophen-3-ylmethyl)-amino]-phenyl}-carbamic acid ethyl ester; {2-Amino-4-[(5-dimethyl-amino-benzo[b]thiophen-3-ylmethyl)-amino]-phenyl}-carbamic acid ethyl ester; {2-Amino-4-[(5-dimethyl-amino-3-methyl-benzo[b]thiophen-2-ylmethyl)-amino]-phenyl}-carbamic acid ethyl ester; {2-Amino-4-[(5-fluoro-thiophen-2-ylmethyl)-amino]-phenyl}-carbamic acid ethyl ester; {2-Amino-4-[(benzo[b]thiophen-2-ylmethyl)-amino]-phenyl}-carbamic acid propyl ester; {2-Amino-4-[(benzo[b]thiophen-3-ylmethyl)-amino]-phenyl}-carbamic acid propyl ester; N-{2-Amino-4-[(5-chloro-thiophen-2-ylmethyl)amino]phenyl}-2-(4-fluoro-phenyl)-acetamide; N-{2-Amino-4-[(5-chloro-thiophen-2-ylmethyl)amino]phenyl}-3,3-dimethyl-butyramide; and pharmaceutically acceptable salts thereof.

In further embodiments, the Kv7 channel activator is a compound according to the formula:

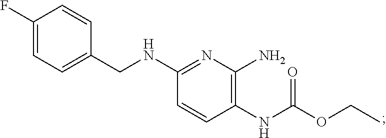 [SMILES: CCOC(=O)Nc1ccc(NCc2ccc(F)cc2)nc1N]

or a pharmaceutically acceptable salt thereof.

In further embodiments, the Kv7 channel activator is selected from the group consisting of: 2-Cyclopentyl-N-(2,6-dimethyl-4-morpholin-4-yl-phenyl)-acetamide; N-(2,6-Dimethyl-4-morpholin-4-yl-phenyl)-3,3-dimethyl-butyramide; N-(4,6-Dimethyl-2-morpholin-4-yl-pyrimidin-5-yl)-2-(4-fluoro-phenyl)-acetamide; Hexanoic acid (2,6-difluoro-4-morpholin-4-yl-phenyl)-amide; 2-Cyclopentyl-N-(4,6-dimethyl-2-morpholin-4-yl-pyrimidin-5-yl)-acetamide; N-(2-Bromo-4-morpholin-4-yl-6-trifluoromethyl-phenyl)-propionamide; N-(2,4-Dimethyl-6-morpholin-4-yl-pyridin-3-yl)-3,3-dimethyl-butyramide; [2-Amino-4-(2,4,6-trimethyl-benzylamino)-phenyl]-carbamic acid ethyl ester; 2-Cyclopentyl-N-(2-methoxy-6-methyl-4-morpholin-4-yl-phenyl)-acetamide; and pharmaceutically acceptable salts thereof.

In further embodiments, the Kv7 channel activator is a compound according to formula 10 wherein: R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl and hydroxy-C₃₋₈-cycloalk(en)yl; R² and R^{2′} are independently selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, hydroxy-C₁₋₆-alk(en/yn)yl and hydroxy-C₃₋₈-cycloalk(en)yl; R³ is selected from the group consisting of hydrogen, C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, hydroxy-C₁₋₆-alk(en/yn)yl, aryl-C₃₋₈-cycloalk(en)yl, NR¹⁰R^{10′}-C₁₋₆-alk(en/yn)yl, NR¹⁰R^{10′}-C₃₋₈-cycloalk(en)yl and hydroxy-C₃₋₈-cycloalk(en)yl, wherein: R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or R¹⁰ and R^{10′} together with the nitrogen atom to which they are attached form a 4-8 membered saturated or unsaturated ring that optionally contains 1, 2 or 3 further heteroatoms; X is CO or SO₂; Z is O or NR⁴, wherein: R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl; or R³ and R⁴ together with the nitrogen atom to which they are attached form a 4-8 membered saturated or unsaturated ring that optionally contains 1, 2 or 3 further heteroatoms, the ring formed by R³ and R⁴ and the nitrogen atom is optionally substituted with one or more substituents independently selected from C₁₋₆-alk(en/yn)yl, aryl and aryl-C₁₋₆-alk(en/yn)yl; q is 0 or 1; and Y represents a heteroaryl of formula:

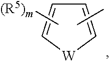

wherein: W is S; m is 0, 1, 2 or 3; n is 0, 1, 2, 3 or 4; p is 0 or 1; and each R⁵ is independently selected from the group consisting of C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, halogen, halo-C₁₋₆-alk(en/yn)yl, C₁₋₆-alk(en/yn)yloxy, —CO—NR⁶R^{6′}, cyano, nitro, —NR⁷R^{7′}, —S—R⁸, —SO₂R⁸, and SO₂OR⁸, wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and aryl; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, aryl and acyl; and R⁸ is selected from the group consisting of C₁₋₆-alk(en/yn)yl, C₃₋₈-cycloalk(en)yl, C₃₋₈-cycloalk(en)yl-C₁₋₆-alk(en/yn)yl, aryl and —NR⁹R^{9′}, wherein: R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; or a pharmaceutically acceptable salt thereof.

In further embodiments, R¹ is selected from the group consisting of hydrogen and C₁₋₆-alk(en/yn)yl.

In further embodiments, at least one of the substituents R² and R^{2′} is a hydrogen atom.

In further embodiments both R² and R^{2′} are hydrogen atoms.

In further embodiments, X is CO.

In further embodiments, q is 0.

In further embodiments, q is 1 and Z is an oxygen atom.

In further embodiments, R³ is selected from the group consisting of C₁₋₆-alk(en/yn)yl and aryl-C₁₋₆-alk(en/yn)yl.

In further embodiments, R³ is C₁₋₆-alk(en/yn)yl.

In further embodiments, R³ is aryl-C₁₋₆-alk(en/yn)yl.

In further embodiments, W is a sulfur atom.

In further embodiments, Y is of formula:

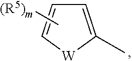

wherein: W is S; m is 0, 1, 2 or 3; n is 0, 1, 2, 3 or 4; p is 0 or 1; and each R⁵ is independently selected from the group consisting of C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, halogen, halo-C₁₋₆-alk(en/yn)yl, C₁₋₆-alk(en/yn)yloxy, —CO—NR⁶R^{6′}, cyano, nitro, —NR⁷R^{7′}, —S—R⁸, —SO₂R⁸, and SO₂OR⁸, wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and aryl; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, aryl and acyl; and R⁸ is selected from the group consisting of C₁₋₆-alk(en/yn)yl, C₃₋₈-cycloalk(en)yl, C₃₋₈-cycloalk(en)yl-C₁₋₆-alk(en/yn)yl, aryl and —NR⁹R^{9′}, wherein: R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl.

In further embodiments, Y is of formula:

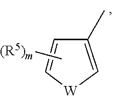

wherein: W is S; m is 0, 1, 2 or 3; n is 0, 1, 2, 3 or 4; p is 0 or 1; and each R⁵ is independently selected from the group consisting of C₁₋₆-alk(en/yn)yl, C₃₋₈-cycloalk(en)yl, aryl, C₃₋₈-cycloalk(en)yl-C₁₋₆-alk(en/yn)yl, aryl-C₁₋₆-alk(en/yn)yl, acyl, halogen, halo-C₁₋₆-alk(en/yn)yl, C₁₋₆-alk(en/yn)yloxy, —CO—NR⁶R^{6′}, cyano, nitro, —NR⁷R^{7′}, —S—R⁸, —SO₂R⁸, and SO₂OR⁸, wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and aryl; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, aryl and acyl; and R⁸ is selected from the group consisting of C₁₋₆-alk(en/yn)yl, C₃₋₈-cycloalk(en)yl, C₃₋₈-cycloalk(en)yl-C₁₋₆-alk(en/yn)yl, aryl and —NR⁹R^{9′}, wherein: R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl.

### Formula 11

In an embodiment, the Kv7 channel activator may be selected from one of the following compounds according to Formula 11. Such compounds are described in International Publication No. WO2009015667A1, published Feb. 5, 2009, and corresponding to International Application No. PCT/DK2008/050191 filed Jul. 31, 2008; International Publication No. WO2004058739A1, published Jul. 15, 2004 and corresponding to International Application No. PCT/DK2003/000906 filed Dec. 18, 2003; U.S. Pat. No. 7,368,472 issued May 6, 2008 and corresponding to U.S. application Ser. No. 10/540,075 filed Dec. 18, 2003; US Publication No. US20100256145A1, published Oct. 7, 2010 and corresponding to U.S. application Ser. No. 12/671,505 filed Jul. 31, 2008; International Publication No. WO2004082677A1, published Sep. 30, 2004 and corresponding to International Application No. PCT/DK2004/000186 filed Mar. 18, 2004; International Publication No. WO2004080950A1, published or issued Sep. 23, 2004 and corresponding to International Application No. PCT/DK2004/000162 filed Mar. 12, 2004; which are incorporated by reference in their entirety herein. In the case of any conflict of terminology in the context of Formula 11, these references incorporated by reference herein control.

In an embodiment, the Kv7 channel activator is a compound according to formula 11:

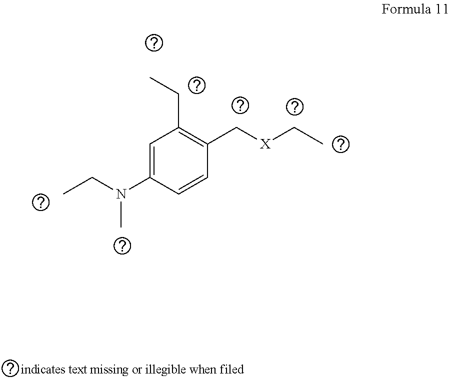

wherein: s is 0 or 1; U is O, S, SO₂, SO₂NR¹¹, CO—O or CONR¹¹; wherein: R¹¹ is selected from the group consisting of hydrogen, C₁₋₆-alk(en/yn)yl, C₃₋₈-cycloalk(en)yl, and C₃₋₈-cycloalk(en)yl-C₁₋₆-alk(en/yn)yl; or R² and R¹¹ together with the nitrogen atom to which R¹¹ is attached form a 5-8 membered saturated or unsaturated ring that optionally contains 1, 2 or 3 further heteroatoms; q is 0 or 1; X is CO or SO₂; with the proviso when X is SO₂, then q is 0; Z is O or S; R¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR¹⁰R^{10′}-C₁₋₆-alk(en/yn)yl, NR¹⁰R^{10′}-C₃₋₈-cycloalk(en)yl and NR¹⁰R^{10′}-C₃₋₈-cycloalk(en)yl-C₁₋₆-alk(en/yn)yl; wherein: R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yl; or R¹⁰ and R^{10′} together with the nitrogen atom to which they are attached form a 5-8 membered saturated or unsaturated ring that optionally contains 1, 2 or 3 further heteroatoms; and provided that: when R² is halogen or cyano, then s is 0; and when s is 1 and R² is a hydrogen atom or acyl, then U is O or S; R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, heterocycloalk(en)yl-C₁₋₆-alk(en/yn)yl, Ar, Ar-C₁₋₆-alk(en/yn)yl, Ar-C₃₋₈-cycloalk(en)yl, Ar-heterocycloalk(en)yl, Ar-C₃₋₈-cycloalk(en)yl-C₁₋₆-alk(en/yn)yl, Ar-C₁₋₆-alk(en/yn)yl-C₃₋₈-cycloalk(en)yl, Ar-C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar-C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yl oxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, NR¹²R^{12′}, optionally substituted NR¹²R^{12′}-C₁₋₆-alk(en/yn)yl, optionally substituted NR¹²R^{12′}-C₃₋₈-cycloalk(en)yl, and optionally substituted NR¹²R^{12′}-C₃₋₈-cycloalk(en)yl-C₁₋₆-alk(en/yn)yl; wherein: R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, Ar-heterocycloalk(en)yl, Ar-oxy-C₁₋₆-alk(en/yn)yl, Ar-oxy-C₃₋₈-cycloalk(en)yl, Ar-oxy-C₃₋₈-cycloalk(en)yl-C₁₋₆-alk(en/yn)yl, Ar-oxy-heterocycloalk(en)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yr) yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹² and R^{12′} together with the nitrogen atom to which they are attached form a 5-8 membered saturated or unsaturated ring that optionally contains 1, 2 or 3 further heteroatoms; provided that: when R³ is NR¹²R^{12′}, then q is 0; and Y represents a group of the following formulas:

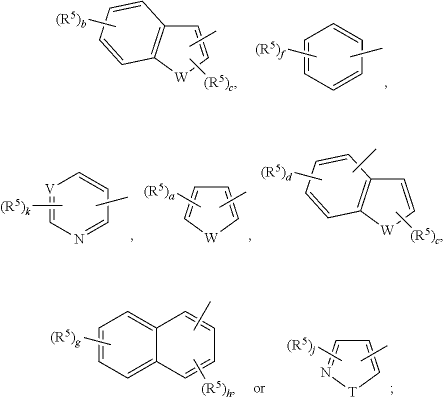

wherein: the line represents a bond attaching the group represented by Y to the carbon atom; W is O or S; Vis N, C or CH; T is N, NH or O; a is 0, 1, 2 or 3; b is 0, 1, 2, 3 or 4; c is 0 or 1; d is 0, 1, 2 or 3; e is 0, 1 or 2; f is 0, 1, 2, 3, 4 or 5; g is 0, 1, 2, 3 or 4; h is 0, 1, 2 or 3; j is 0, 1 or 2; k is 0, 1, 2 or 3; and each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, Ar-oxy, Ar-oxy-C₁₋₆-alk(en/yn)yl, Ar-oxy-C₃₋₈-cycloalk(en)yl, C₁₋₆-alk(en/yn)yl-heterocycloalk(en)yl, Ar-oxy-C₃₋₈-cycloalk(en)yl-C₁₋₆-alk(en/yn)yl, acyl, C₁₋₆-alk(en/yn)yloxy, C₃₋₈-cycloalk(en)yloxy, C₃₋₈-cycloalk(en)yl-C₁₋₆-alk(en/yn)yloxy, C₁₋₆-alk(en/yn)yloxy-carbonyl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR⁷R^{7′}, S—R⁸ and SO₂R⁸; or two adjacent R⁵ together with the aromatic group form a 5-8 membered ring that optionally contains one or two heteroatoms; wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, heterocycloalk(en)yl-C₁₋₆-alk(en/yn)yl, heterocycloalk(en)yl-C₃₋₈-cycloalk(en)yl, heterocycloalk(en)yl-C₃₋₈-cycloalk(en)yl-C₁₋₆-alk(en/yn)yl, heterocycloalk(en)yl-Ar and acyl; or R⁷ and R^{7′} together with the nitrogen atom to which they are attached form a 5-8 membered saturated or unsaturated ring that optionally contains 1, 2 or 3 further heteroatoms; and R⁸ is selected from the group consisting of hydrogen, C₁₋₆-alk(en/yn)yl, C₃₋₈-cycloalk (en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; or a pharmaceutically acceptable salt thereof.

In further embodiments, the Kv7 channel activator is selected from the group consisting of: {4-[(Benzofuran-2-ylmethyl)-amino]-2-methylphenyl}-carbamic acid propyl ester; {4-[(5-Chloro-thiophen-2-ylmethyl)-amino]-2-methylphenyl}-carbamic acid ethyl ester; {4-[(Benzo[b]thiophen-2-ylmethyl)-amino]-2-methylphenyl}-carbamic acid ethyl ester; {2-Methyl-4-[(5-phenyl-thiophen-2-ylmethyl)-amino]-phenyl}-carbamic acid ethyl ester; [4-(4-Isopropyl-benzylamino)-2-methylphenyl]-carbamic acid ethyl ester; [4-(4-Fluoro-benzylamino)-2-methylphenyl]-carbamic acid propyl ester; (4-{[4-(4-Chloro-benzenesulfonyl)-3-methyl-thiophen-2-ylmethyl]-amino}-2-methylphenyl)-carbamic acid propyl ester; {4-[(5-Methyl-thiophen-2-ylmethyl)-amino]-2-methylphenyl}-carbamic acid propyl ester; {4-[(5-Bromo-thiophen-2-ylmethyl)-amino]-2-methylphenyl}-carbamic acid propyl ester; {4-[(5-Chloro-thiophen-2-ylmethyl)-amino]-2-methylphenyl}-carbamic acid propyl ester; {4-[(Benzo[b]thiophen-2-ylmethyl)-amino]-2-methylphenyl}-carbamic acid propyl ester; {2-Methyl-4-[(5-phenyl-thiophen-2-ylmethyl)-amino]-phenyl}-carbamic acid propyl ester; [4-(4-Isopropyl-benzylamino)-2-methylphenyl]-carbamic acid propyl ester; {4-[(5-Bromo-thiophen-2-ylmethyl)-amino]-2-chlorophenyl}-carbamic acid ethyl ester; {4-[(5-Chloro-thiophen-2-ylmethyl)-amino]-2-chlorophenyl}-carbamic acid ethyl ester; {4-[(Benzo[b]thiophen-2-ylmethyl)-amino]-2-chlorophenyl}-carbamic acid ethyl ester; [2-Chloro-4-(4-isopropyl-benzylamino)-phenyl]-carbamic acid ethyl ester; [2-Chloro-4-(4-fluoro-benzylamino)-phenyl]-carbamic acid propyl ester; 2-Chloro-4-{[4-(4-chloro-benzenesulfonyl)-3-methyl-thiophen-2-ylmethyl]-amino)-phenyl}-carbamic acid propyl ester; {4-[(5-Methyl-thiophen-2-ylmethyl)-amino]-2-chlorophenyl}-carbamic acid propyl ester; {4-[(5-Bromo-thiophen-2-ylmethyl)-amino]-2-chlorophenyl}-carbamic acid propyl ester; {2-Chloro-4-[(5-chloro-thiophen-2-ylmethyl)-amino]-phenyl}-carbamic acid propyl ester; {4-[(Benzo[b]thiophen-2-ylmethyl)-amino]-2-chlorophenyl}-carbamic acid propyl ester; {4-[(Benzofuran-2-ylmethyl)-amino]-2-chlorophenyl}-carbamic acid propyl ester; {4-[(5-Chloro-thiophen-2-ylmethyl)-amino]-2-cyanophenyl}-carbamic acid ethyl ester; {4-[(Benzo[b]thiophen-2-ylmethyl)-amino]-2-methoxyphenyl}-carbamic acid methyl ester; {4-[(5-Bromo-thiophen-2-ylmethyl)-amino]-2-methoxyphenyl}-carbamic acid isopropyl ester; {4-[(4-Fluoro-benzyl)-(methyl)amino]-2-methoxyphenyl}-carbamic acid propyl ester; [4-(Benzo[b]thiophen-2-ylmethyl-(methyl)amino)-2-methoxy-phenyl]-carbamic acid propyl ester; {4-[(5-Chloro-thiophen-2-ylmethyl)-(methyl)amino]-2-methoxy-phenyl}-carbamic acid propyl ester; {4-[(5-Bromo-thiophen-2-ylmethyl)-(methyl)amino]-2-methoxy-phenyl}-carbamic acid propyl ester; {2-Methoxy-4-[methyl-(5-methyl-thiophen-2-ylmethyl)-amino]-phenyl}-carbamic acid propyl ester; {4-[(4-Fluorobenzyl)-(methyl)-amino]-2-isopropoxyphenyl}-carbamic acid ethyl ester; [4-(3-Fluorobenzylamino)-2-methoxyphenyl]-carbamic acid ethyl ester; [4-(4-Isopropylbenzylamino)-2-methoxyphenyl]-carbamic acid ethyl ester; {2-Methoxy-4-[(3-methylthiophen-2-ylmethyl)-amino]-phenyl}-carbamic acid ethyl ester; [4-(2,4-Difluorobenzylamino)-2-methoxyphenyl]-carbamic acid ethyl ester; [2-Cyclopentyloxy-4-(4-methoxybenzylamino)-phenyl]-carbamic acid ethylester; [2-Cyclopentyloxy-4-(3-fluoro-2-methylbenzylamino)-phenyl]-carbamic acid ethyl ester; [4-(3-Fluoro-2-methylbenzylamino)-2-phenethyloxyphenyl]-carbamic acid ethyl ester; [2-Benzyloxy-4-(3-fluoro-2-methylbenzylamino)-phenyl]carbamic acid ethyl ester; [2-Benzyloxy-4-(4-methylsulfanylbenzylamino)-phenyl]-carbamic acid ethyl ester; {4-[(Benzo[b]thiophen-3-ylmethyl)-amino]-2-cyclopentyloxyphenyl}-carbamic acid ethyl ester; [4-(3-Fluoro-2-methylbenzylamino)-2-isopropoxyphenyl]-carbamic acid ethyl ester; [2-Benzyloxy-4-(3-methoxybenzylamino)-phenyl]-carbamic acid ethyl ester; {4-[(Benzo[1,3]dioxol-5-ylmethyl)-amino]-2-isopropoxyphenyl}-carbamic acid ethyl ester; {4-[(5-Bromo-thiophen-2-ylmethyl)-amino]-phenyl}-carbamic acid propyl ester; {4-[(5-Chloro-thiophen-2-ylmethyl)-amino]-phenyl}-carbamic acid propyl ester; [2-Cyano-4-(4-isopropylbenzylamino)-phenyl]-carbamic acid ethyl ester; {4-[(5-Bromo-thiophen-2-ylmethyl)-(methyl)amino]-2-methyl phenyl}-carbamic acid propyl ester; {4-[(4-Isopropylbenzyl)-(methyl)amino]-2-methyl phenyl}-carbamic acid propyl ester; {2-Methyl-4-[methyl-(4-trifluoromethyl-benzyl)-amino]-phenyl}-carbamic acid propyl ester; {2-Methyl-4-[methyl-(4-methylsulfanyl-benzyl)-amino]-phenyl}-carbamic acid propyl ester; {4-[(4-tert-Butyl-benzyl)-(methyl)amino]-2-chlorophenyl}-carbamic acid ethyl ester; {2-Chloro-4-[methyl-(4-trifluoromethyl-benzyl)-amino]-phenyl}-carbamic acid ethyl ester; {2-Chloro-4-[methyl-(4-methylsulfanyl-benzyl)-amino]-phenyl}-carbamic acid ethyl ester; {4-[(5-Bromo-thiophen-2-ylmethyl)-(methyl)amino]-2-chlorophenyl}-carbamic acid propyl ester; {2-Chloro-4-[(5-chloro-thiophen-2-ylmethyl)-(methyl)amino]-phenyl}-carbamic acid propyl ester; {4-[(4-tert-Butyl-benzyl)-(methyl)amino]-2-chlorophenyl}-carbamic acid propyl ester; {2-Chloro-4-[methyl-(4-trifluoromethyl-benzyl)-amino]-phenyl}-carbamic acid propyl ester; {4-[(5-Bromo-thiophen-2-ylmethyl)-(methyl)amino]-2-trifluoromethyl-phenyl}-carbamic acid ethyl ester; {4-[(5-Chloro-thiophen-2-ylmethyl)-(methyl)amino]-2-trifluoromethyl-phenyl}-carbamic acid ethyl ester; {4-[(4-Isopropyl-benzyl)-(methyl)amino]-2-trifluoromethyl-phenyl}-carbamic acid ethyl ester; {4-[(4-tert-Butyl-benzyl)-(methyl)amino]-2-trifluoromethyl-phenyl}-carbamic acid ethyl ester; {4-[Methyl-(4-trifluoromethyl-benzyl)-amino]-2-trifluoromethyl-phenyl}-carbamic acid ethyl ester; {4-[Methyl-(4-methylsulfanyl-benzyl)-amino]-2-trifluoromethyl-phenyl}-carbamic acid ethyl ester; {4-[(5-Bromo-thiophen-2-ylmethyl)-(methyl)amino]-2-trifluoromethyl-phenyl}-carbamic acid propyl ester; {4-[(5-Chloro-thiophen-2-ylmethyl)-(methyl)amino]-2-trifluoromethyl-phenyl}-carbamic acid propyl ester; {4-[(4-Isopropyl-benzyl)-(methyl)amino]-2-trifluoromethyl-phenyl}-carbamic acid propyl ester; {4-[(4-tert-Butyl-benzyl)-(methyl)amino]-2-trifluoromethyl-phenyl}-carbamic acid propyl ester; {4-[Methyl-(4-trifluoromethyl-benzyl)-amino]-2-trifluoromethyl-phenyl}-carbamic acid propyl ester; {4-[Methyl-(4-methylsulfanyl-benzyl)-amino]-2-trifluoromethyl-phenyl}-carbamic acid propyl ester; {4-[(5-Bromo-thiophen-2-ylmethyl)-(methyl)amino]-2-cyanophenyl}-carbamic acid propyl ester; {4-[(4-tert-Butyl-benzyl)-(methyl)amino]-2-cyanophenyl}-carbamic acid propyl ester; {2-Cyano-4-[methyl-(4-trifluoromethyl-benzyl)-amino]-phenyl}-carbamic acid propyl ester; {2-Bromo-4-[(5-bromo-thiophen-2-ylmethyl)-(methyl)amino]phenyl}-carbamic acid propyl ester; {2-Bromo-4-[(5-chloro-thiophen-2-ylmethyl)-(methyl)amino]-phenyl}-carbamic acid propyl ester; {2-Bromo-4-[(4-isopropylbenzyl)-(methyl)amino]-phenyl}-carbamic acid propyl ester; {2-Bromo-4-[(4-tert-butyl-benzyl)-(methyl)amino]-phenyl}-carbamic acid propyl ester; {2-Bromo-4-[methyl-(4-trifluoromethyl-benzyl-amino]-phenyl}-carbamic acid propyl ester; [2-Iodo-4-(4-isopropyl-benzylamino)-phenyl]-carbamic acid propyl ester; [4-(4-tert-Butyl-benzylamino)-2-iodophenyl]-carbamic acid propyl ester; [2-Iodo-4-(4-trifluoromethyl-benzylamino)-phenyl]-carbamic acid propyl ester; [2-Iodo-4-(4-methylsulfanyl-benzylamino)-phenyl]-carbamic acid propyl ester; {2-Iodo-4-[4-(4-methylpiperazin-1-yl)-benzylamino]-phenyl}-carbamic acid propyl ester; {4-[(5-Bromo-thiophen-2-ylmethylyamino]-2-trifluoromethyl-phenyl}-carbamic acid ethyl ester; {4-[(5-Chloro-thiophen-2-ylmethyl)-amino]-2-trifluoromethyl-phenyl}-carbamic acid ethyl ester; [4-(4-tert-Butyl-benzylamino)-2-trifluoromethyl-phenyl]-carbamic acid ethyl ester; [4-(4-Methylsulfanyl-benzylamino)-2-trifluoromethyl-phenyl]-carbamic acid ethyl ester; {4-[(5-Bromo-thiophen-2-ylmethyl)-amino]-2-trifluoromethyl-phenyl}-carbamic acid propyl ester; [4-(4-Isopropylbenzylamino)-2-trifluoromethyl-phenyl]-carbamic acid propyl ester; [4-(4-tert-Butyl-benzylamino)-2-trifluoromethyl-phenyl]-carbamic acid propyl ester; [2-Trifluoromethyl-4-(4-trifluoromethyl-benzylamino)-phenyl]-carbamic acid propyl ester; [4-(4-Dimethylamino-benzylamino)-2-trifluoromethyl-phenyl]-carbamic acid propyl ester; [4-(4-Methylsulfanyl-benzylamino)-2-trifluoromethyl-phenyl]-carbamic acid propyl ester; {4-[(5-Bromo-thiophen-2-ylmethyl)-amino]-2-cyanophenyl}-carbamic acid propyl ester; {4-[(5-Chloro-thiophen-2-ylmethyl)-amino]-2-cyanophenyl}-carbamic acid propyl ester; [2-Cyano-4-(4-trifluoromethyl-benzylamino)-phenyl]-carbamic acid propyl ester; {2-Bromo-4-[(5-bromo-thiophen-2-ylmethyl)-amino]-phenyl}-carbamic acid propyl ester; {2-Bromo-4-[(5-chloro-thiophen-2-ylmethyl)-amino]-phenyl}-carbamic acid propyl ester; [2-Bromo-4-(4-isopropylbenzylamino)-phenyl]-carbamic acid propyl ester; [2-Bromo-4-(4-tert-butyl-benzylamino)-phenyl]-carbamic acid propyl ester; [2-Bromo-4-(4-trifluoromethyl-benzylamino)-phenyl]carbamic acid propyl ester; [2-Bromo-4-(4-methylsulfanyl-benzylamino)-phenyl]-carbamic acid propyl ester; N-{4-[(5-Bromo-thiophen-2-ylmethyl)-amino]-2-methoxyphenyl}-butyramide; N-{4-[(5-Chloro-thiophen-2-ylmethyl)-amino]-2-methoxyphenyl}-butyramide; N-[4-(4-Isopropylbenzylamino)-2-methoxyphenyl]-butyramide; N-[4-(4-tert-Butyl-benzylamino)-2-methoxyphenyl]-butyramide; N-[2-Methoxy-4-(4-trifluoromethyl-benzylamino)-phenyl]-butyramide; {4-[(5-Chloro-thiophen-2-ylmethyl)-amino]-2-furan-2-yl-phenyl}-carbamic acid propyl ester; [2-Furan-2-yl-4-(4-isopropylbenzylamino)-phenyl]-carbamic acid propyl ester; [5-(4-Fluorobenzylamino)-biphenyl-2-yl]-carbamic acid propyl ester; {5-[(5-Chloro-thiophen-2-ylmethyl)-amino]-biphenyl-2-yl}-carbamic acid propyl ester; [5-(4-Isopropylbenzylamino)-biphenyl-2-yl]-carbamic acid propyl ester; N-{2-Chloro-4-[(5-chloro-thiophen-2-ylmethyl)-(methyl)amino]-phenyl}-2-phenylacetamide; N-{2-Chloro-4-[(5-chloro-thiophen-2-ylmethyl)-(methyl)amino]-phenyl}-3,3-dimethylbutyramide; N-{2-Chloro-4-[(5-chloro-thiophen-2-ylmethyl)-(methyl)amino]-phenyl}-3-phenylpropionamide; N-{2-Chloro-4-[(5-chloro-thiophen-2-ylmethyl)-(methyl)amino]-phenyl}-butyramide; Pentanoic acid {2-chloro-4-[(5-chloro-thiophen-2-ylmethyl)-(methyl)amino]-phenyl}-amide; Cyclopropanecarboxylic acid {2-chloro-4-[(5-chloro-thiophen-2-ylmethyl)-(methyl)amino]-phenyl}-amide; Cyclobutanecarboxylic acid {2-chloro-4-[(5-chloro-thiophen-2-ylmethyl)-(methyl)amino]-phenyl}-amide; Cyclopentanecarboxylic acid {2-chloro-4-[(5-chloro-thiophen-2-ylmethyl)-(methyl)amino]-phenyl}-amide; Cyclohexanecarboxylic acid {2-chloro-4-[(5-chloro-thiophen-2-ylmethyl)-(methyl)amino]-phenyl}-amide; N-{2-Chloro-4-[(5-chloro-thiophen-2-ylmethyl)-(methyl)amino]-phenyl}-2-thiophen-2-yl-acetamide; N-{2-Chloro-4-[(5-chloro-thiophen-2-ylmethyl)-(methyl)amino]-phenyl}-2-(3-methoxy-phenyl)-acetamide, N-{2-Chloro-4-[(5-chloro-thiophen-2-ylmethyl)-(methyl)amino]-phenyl)-2-(4-chloro-phenyl}-acetamide; N-{2-Chloro-4-[(5-chloro-thiophen-2-ylmethyl)-(methyl)amino]-phenyl}-2-(4-methoxy-phenyl)-acetamide; N-{2-Chloro-4-[(5-chloro-thiophen-2-ylmethyl)-(methyl)amino]-phenyl)-2-(4-fluoro-phenyl}-acetamide; N-{2-Chloro-4-[(5-chloro-thiophen-2-ylmethyl)-(methyl)amino]-phenyl}-3-cyclohexylpropionamide; N-{2-Chloro-4-[(5-chloro-thiophen-2-ylmethyl)-amino]-phenyl}-2,2-dimethylpropionamide; N-{2-Chloro-4-[(5-chloro-thiophen-2-ylmethyl)-amino]-phenyl}-2-phenoxyacetamide; N-{2-Chloro-4-[(5-chloro-thiophen-2-ylmethyl)-amino]-phenyl}-2-phenyl acetamide; N-{2-Chloro-4-[(5-chloro-thiophen-2-ylmethyl)-amino]-phenyl}-3,3-dimethylbutyramide; N-{2-Chloro-4-[(5-chloro-thiophen-2-ylmethyl)-amino]-phenyl}-butyramide; Pentanoic acid {2-chloro-4-[(5-chloro-thiophen-2-ylmethyl)-amino]-phenyl}-amide; Cyclopropanecarboxylic acid {2-chloro-4-[(5-chloro-thiophen-2-ylmethyl)-amino]-phenyl}-amide; Cyclobutanecarboxylic acid {2-chloro-4-[(5-chloro-thiophen-2-ylmethyl)-amino]-phenyl}-amide; Cyclopentanecarboxylic acid {2-chloro-4-[(5-chloro-thiophen-2-ylmethyl)-amino]phenyl}-amide; Cyclohexanecarboxylic acid {2-chloro-4-[(5-chloro-thiophen-2-ylmethyl)-amino]-phenyl}-amide; N-{2-Chloro-4-[(5-chloro-thiophen-2-ylmethyl)-amino]-phenyl}-2-thiophen-2-yl-acetamide; N-{2-Chloro-4-[(5-chloro-thiophen-2-ylmethyl)-amino]-phenyl}-2-(3-methoxyphenyl)-acetamide; N-{2-Chloro-4-[(5-chloro-thiophen-2-ylmethyl)-amino]-phenyl}-2-(4-chlorophenyl)-acetamide; N-{2-Chloro-4-[(5-chloro-thiophen-2-ylmethyl)-amino]-phenyl}-2-(4-methoxyphenyl)-acetamide; N-{2-Chloro-4-[(5-chloro-thiophen-2-ylmethyl)-amino]-phenyl}-2-(4-fluorophenyl)-acetamide; 2,3-Dihydro-benzo[1,4]dioxine-6-carboxyl acid {2-chloro-4-[(5-chloro-thiophen-2-ylmethyl)-amino]-phenyl}-amide; 2,3-Dihydro-benzofuran-5-carboxylic acid {2-chloro-4-[(5-chloro-thiophen-2-ylmethyl)-amino]-phenyl}-amide; N-{2-Chloro-4-[(5-chloro-thiophen-2-ylmethyl)-amino]-phenyl}-3-cyclohexylpropionamide; N-{4-[(5-Chloro-thiophen-2-ylmethyl)-(methyl)amino]-2-methyl-phenyl}-2,2-dimethylpropionamide; N-{4-[(5-Chloro-thiophen-2-ylmethyl)-(methyl)amino]-2-methyl-phenyl}-2-phenyl acetamide; N-{4-[(5-Chloro-thiophen-2-ylmethyl)-(methyl)amino]-2-methyl-phenyl}-3,3-dimethylbutyramide; N-{4-[(5-Chloro-thiophen-2-ylmethyl)-(methyl)amino]-2-methyl-phenyl}-3-phenylpropionamide; N-{4-[(5-Chloro-thiophen-2-ylmethyl)-(methyl)amino]-2-methyl-phenyl}-butyramide; 2,2,2-Trichloro-N-{4-[(5-chloro-thiophen-2-ylmethyl)-(methyl)amino]-2-methyl-phenyl}-acetamide; Cyclopropanecarboxylic acid {4-[(5-chloro-thiophen-2-ylmethyl)-(methyl)amino]-2-methyl-phenyl}-amide; Cyclobutanecarboxylic acid {4-[(5-chloro-thiophen-2-ylmethyl)-(methyl)amino]-2-methylphenyl}-amide; Cyclopentanecarboxylic acid {4-[(5-chloro-thiophen-2-ylmethyl)-(methyl)amino]-2-methyl phenyl}-amide; Cyclohexanecarboxylic acid {4-[(5-chloro-thiophen-2-ylmethyl)-(methyl)amino]-2-methylphenyl}-amide; N-{4-[(5-Chloro-thiophen-2-ylmethyl)-(methyl)amino]-2-methylphenyl}-2-thiophen-2-yl-acetamide; N-{4-[(5-Chloro-thiophen-2-ylmethyl)-(methyl)amino]-2-methylphenyl}-2-(3-methoxyphenyl)-acetamide; N-{4-[(5-Chloro-thiophen-2-ylmethyl)-(methyl)amino]-2-methyl phenyl}-malonamic acid methyl ester; 2-(4-Chlorophenyl)-N-{4-[(5-chloro-thiophen-2-ylmethyl)-(methyl)amino]-2-methylphenyl}-acetamide; N-{4-[(5-Chloro-thiophen-2-ylmethyl)-(methyl)amino]-2-methyl phenyl}-2-(4-methoxyphenyl)-acetamide; N-{4-[(5-Chloro-thiophen-2-ylmethyl)-(methyl)amino]-2-methyl phenyl}-2-(4-fluorophenyl)-acetamide; N-{4-[(5-Chloro-thiophen-2-ylmethyl)-(methyl)amino]-2-methylphenyl}-3-cyclohexylpropionamide; {2-Chloro-4-[(5-chloro-thiophen-2-ylmethyl)-(methyl)amino]-phenyl}-carbamic acid phenyl ester; {2-Chloro-4-[(5-chloro-thiophen-2-ylmethyl)-(methyl)amino]-phenyl}-carbamic acid benzyl ester; {2-Chloro-4-[(5-chloro-thiophen-2-ylmethyl)-(methyl)amino]-phenyl}-carbamic acid isobutyl ester; {2-Chloro-4-[(5-chloro-thiophen-2-ylmethyl)-(methyl)amino]-phenyl}-carbamic acid butyl ester; {2-Chloro-4-[(5-chloro-thiophen-2-ylmethyl)-(methyl)amino]-phenyl}-carbamic acid hexyl ester; {2-Chloro-4-[(5-chloro-thiophen-2-ylmethyl)-(methyl)amino]-phenyl}-carbamic acid 4-nitrobenzyl ester; {2-Chloro-4-[(5-chloro-thiophen-2-ylmethyl)-(methyl)amino]-phenyl}-carbamic acid but-3-enyl ester; {2-Chloro-4-[(5-chloro-thiophen-2-ylmethyl)-(methyl)amino]-phenyl}-carbamic acid but-2-ynyl ester; {2-Chloro-4-[(5-chloro-thiophen-2-ylmethyl)-(methyl)amino]-phenyl}-carbamic acid 2,2-dimethylpropyl ester; {2-Chloro-4-[(5-chloro-thiophen-2-ylmethyl)-(methyl)amino]-phenyl}-carbamic acid 2-chlorobenzyl ester; {2-Chloro-4-[(5-chloro-thiophen-2-ylmethyl)-(methyl)amino]-phenyl}-carbamic acid 3-chloropropyl ester; {2-Chloro-4-[(5-chloro-thiophen-2-ylmethyl)-(methyl)amino]-phenyl}-carbamic acid 2-benzyloxyethyl ester; 3-{2-Chloro-4-[(5-chloro-thiophen-2-ylmethyl)-(methyl)amino]-phenyl}-1-methyl-1-propyl-urea; 1-{2-Chloro-4-[(5-chloro-thiophen-2-ylmethyl)-(methyl)amino]-phenyl}-3-(2-fluorophenyl)-urea; N-{2-Chloro-4-[(5-chloro-thiophen-2-ylmethyl)-(methyl)amino]-phenyl}-2,2,2-trifluoroacetamide; N-{2-Chloro-4-[(5-chloro-thiophen-2-ylmethyl)-amino]-phenyl}-2,2,2-trifluoroacetamide; N-{5-[(5-Chloro-thiophen-2-ylmethyl)-amino]-4′-dimethylamino-biphenyl-2-yl}-2-(4-fluorophenyl)-acetamide; N-{2-Chloro-4-[(5-chloro-thiophen-2-ylmethyl)-(methyl)amino]-phenyl}-2-(4-chlorophenyl)-acetamide; [4-(3-Fluoro-4-trifluoromethyl-benzylamino)-2-methylphenyl]-carbamic acid ethyl ester; 2-(4-Fluorophenyl)-N-{2-methyl-4-[(6-p-tolyloxypyridin-3-ylmethyl)-amino]-phenyl}-acetamide; N-[2-Methyl-4-(4-trifluoromethyl-benzylamino)-phenyl]-butyramide; 2-(4-Fluorophenyl)-N-{2-methyl-4-[6-trifluoromethylpyridin-3-ylmethyl)-amino]-phenyl}-acetamide; Pentanoic acid {4-[(5-chloro-thiophen-2-ylmethyl)-(methyl)amino]-2-methylphenyl}-amide; 3,3-Dimethyl-N-{2-methyl-4-[(6-p-tolyloxypyridin-3-ylmethyl)-amino]-phenyl}-butyramide; [2-Methyl-4-(4-trifluoromethyl-benzylamino)-phenyl]-carbamic acid ethyl ester; N-{2-Chloro-4-[(5-chloro-thiophen-2-ylmethyl)-(methyl)amino]-phenyl}-2-(4-chlorophenyl)-propionamide; [4-(4-Chloro-benzylamino)-2-methylphenyl]-carbamic acid ethyl ester; {4-[(6-Methoxy-benzo[b]thiophen-2-ylmethyl)-amino]-2-methylphenyl}-carbamic acid propyl ester; {4-[(5=Chloro-thiophen-2-ylmethyl)-amino]-2-quinolin-3-yl-phenyl}-carbamic acid ethyl ester; {4-[(5-Dimethylamino-3-methyl-benzo[b]thiophen-2-ylmethyl)-amino]-2-methylphenyl}-carbamic acid propyl ester; 3,3-Dimethyl-N-{2-methyl-4-[(6-trifluoromethylpyridin-3-ylmethyl)-amino]-phenyl}-butyramide; N-(4-{[6-(4-Cyanophenoxy)-pyridin-3-ylmethyl]-amino}-2-methylphenyl)-2-(4-fluorophenyl)-acetamide; {2-Benzyloxy-4-[(4-fluorobenzyl)-(methyl)amino]-phenyl}-thiocarbamic acid S-ethyl ester; {2-Cyclopentyloxy-4-[(4-fluorobenzyl)-(methyl)amino]-phenyl}-thiocarbamic acid S-ethyl ester; N-{4-[(6-Chloropyridin-3-ylmethyl)-amino]-2-methylphenyl}-2-(4-fluorophenyl)-acetamide; {4-[(7-Dimethylamino-benzo[b]thiophen-2-ylmethyl)-amino]-2-methylphenyl}-carbamic acid propyl ester; 1-{2-Cyclopentyloxy-4-[(4-fluorobenzyl)-(methyl)amino]-phenyl}-3-ethyl-urea; 2-Amino-4-methyl-pentanoic acid [2-methyl-4-(4-trifluoromethyl-benzylamino)-phenyl]-amide; {4-[(6-Methoxy-benzo[b]thiophen-2-ylmethyl)-amino]-2-methylphenyl}-carbamic acid ethyl ester; 2-Amino-4-methyl-pentanoic acid [2-methyl-4-(4-trifluoromethyl-benzylamino)-phenyl]-amide; 2-(4-Fluorophenyl)-N-{2-methyl-4-[(4-methyl-2-phenylpyrimidin-5-ylmethyl)-amino]-phenyl}-acetamide, 3,3-Dimethyl-N-{2-methyl-4-[(2-phenylpyrimidin-5-ylmethyl)-amino]-phenyl}-butyramide; {4-[(5-Chloro-thiophen-2-ylmethyl)-amino]-2-pyridin-3-yl-phenyl}-carbamic acid ethyl ester; 1-Amino-cyclopropanecarboxylic acid [2-methyl-4-(4-trifluoromethyl-benzylamino)-phenyl]-amide; {4-[(5-Chloro-thiophen-2-ylmethyl)-amino]-2-pyridin-4-yl-phenyl}-carbamic acid ethyl ester; N-[2-Methyl-4-(4-trifluoromethyl-benzylamino)-phenyl]-2-piperidin-1-yl-acetamide; N-(4-{[5-(4-Chlorophenoxy)-1,3-dimethyl-1H-pyrazol-4-ylmethyl]-amino}-2-methylphenyl)-2,2-dimethylpropionamide; 2,2-Dimethyl-N-{2-methyl-4-[(6-phenoxypyridin-3-ylmethyl)-amino]-phenyl}-propionamide; N-[2-Methyl-4-(4-trifluoromethyl-benzylamino)-phenyl]-2-pyrrolidin-1-yl-acetamide; [4-[(5-Chloro-thiophen-2-ylmethyl)-amino]-2-(6-methoxypyridin-3-yl)-phenyl]-carbamic acid ethyl ester; 4-[(3-Methyl-4-propoxycarbonylamino-phenyl amino)-methyl]-benzoic acid methyl ester; N-[2-Methyl-4-(4-trifluoromethyl-benzylamino)-phenyl]-2-morpholin-4-yl-acetamide; 2,2-Dimethyl-N-{2-methyl-4-[(3-methyl-5-phenylisoxazol-4-ylmethyl)-amino]-phenyl}-propionamide; {4-[(5-Chloro-thiophen-2-ylmethyl-amino]-2-iodophenyl}-carbamic acid ethyl ester; N-{4-[(5-Chloro-thiophen-2-ylmethyl)-amino]-2-iodophenyl}-2-(4-fluorophenyl)-acetamide; {4-[(5-Chloro-thiophen-2-ylmethyl)-amino]-2-quinolin-5-yl-phenyl}-carbamic acid ethyl ester; and pharmaceutically acceptable salts thereof.

In further embodiments, the Kv7 channel activator is a compound according to formula 11, wherein: s is 0 or 1; U is O, S, SO₂, SONR¹¹, or CONR¹¹; wherein: R¹¹ is hydrogen, C₁₋₆-alk(en/yn)yl, C₃₋₈-cycloalk(en)yl, or C₃₋₈-cycloalk(en)yl-C₁₋₆-alk(en/yn)yl; or R² and R¹¹ taken together with the nitrogen atom form a 5-8 membered saturated or unsaturated ring, which optionally contains 1, 2 or 3 further heteroatoms; q is 0 or 1; X is CO or SO₂; with the proviso that q is 0 when X is SO₂; Z is O or S; R¹ is hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl or cyano-C₃₋₈-cycloalk(en)yl-C₁₋₆-alk(en/yn)yl; R² is hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈cycloalk(en)yl, cyano-C₃₋₈-cycloalk(en)yl-C₁₋₆-alk(en/yn)yl, NR¹⁰R^{10′}-C₁₋₆-alk(en/yn)yl, NR¹⁰R^{10′}-C₃₋₈-cycloalk(en)yl or NR¹⁰R^{10′}-C₃₋₈-cycloalk(en)yl-C₁₋₆-alk(en/yn)yl; wherein: R¹⁰ and R^{10′} are each independently hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl or cyano-C₃₋₈-cycloalk(en)yl-C₁₋₆-alk(en/yn)yl; or R¹⁰ and R^{10′} taken together with the nitrogen atom form a 5-8 membered saturated or unsaturated ring, which optionally contains 1, 2 or 3 further heteroatoms; with the proviso that: when R² is halogen or cyano, then s is 0; and when s is 1 and R² is a hydrogen atom or acyl, then U is O or S; R³ is C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, heterocycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, NR¹²R^{12′}, optionally substituted NR¹²R^{12′}-C₁₋₆-alk(en/yn)yl, optionally substituted NR¹²R^{12′}-C₃₋₈-alk(en/yn)yl, or optionally substituted NR¹²R^{12′}-C₃₋₈-alk(en/yn)yl-C₁₋₆-alk(en/yn)yl; wherein: R¹² and R^{12′} are each independently hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, Ar-heterocycloalk(en)yl, Ar-oxy-C₁₋₆-alk(en/yn)yl, Ar-oxy-C₃₋₈-cycloalk(en)yl, Ar-oxy-C₃₋₈-cycloalk(en)yl-C₁₋₆-alk(en/yn)yl, Ar-oxy-heterocycloalk(en)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl, or cyano-C₃₋₈-cycloalk(en)yl-C₁₋₆-alk(en/yn)yl; or R¹² and R^{12′} taken together with the nitrogen atom form a 5-8 membered saturated or unsaturated ring, which optionally contains 1, 2 or 3 further heteroatoms; with the proviso that when R³ is NR¹²R^{12′} then q is 0; and Y is a group of formula:

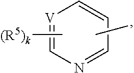

wherein: “|” represents a bond attaching the group represented by Y to the carbon atom; Vis C or CH; and k is 0, 1, 2 or 3; and each R⁵ is independently C₁₋₆-alk(en/yn)yl, C₃₋₈-cycloalk (en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, Ar-oxy, Ar-oxy-C₁₋₆alk(en/yn)yl, Ar-oxy-C₃₋₈-cycloalk(en)yl, C₁₋₆-alk(en/yn)yl-heterocycloalk(en)yl, Ar-oxy-C₃₋₈-cycloalk(en)yl-C₁₋₆-alk(en/yn)yl, acyl, C₁₋₆-alk(en/yn)yloxy, C₃₋₈-cycloalk(en)yloxy, C₃₋₈-cycloalk(en)yl-C₁₋₆-alk(en/yn)yloxy, C₁₋₆-alk(en/yn)yloxy-carbonyl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, NR⁷R^{7′}, S—R⁸ or SO₂R⁸; or two adjacent R⁵ groups taken together with the aromatic group form a 5-8 membered ring, which optionally contains one or two heteroatoms; wherein: R⁶ and R^{6′} are each independently hydrogen, C₁₋₆-alk(en/yn)yl, C₃₋₈-cycloalk(en)yl, C₃₋₈-cycloalk(en)yl-C₁₋₆-alk(en/yn)yl or Ar; R⁷ and R^{7′} are each independently hydrogen, C₁₋₆-alk(en/yn)yl, C₃₋₈-cycloalk(en)yl, C₃₋₈-cycloalk(en)yl-C₁₋₆-alk(en/yn)yl, Ar, heterocycloalk(en)yl-C₁₋₆-alk(en/yn)yl, heterocycloalk(en)yl-C₃₋₈-cycloalk(en)yl, heterocycloalk(en)yl-C₃₋₈-cycloalk(en)yl-C₁₋₆-alk(en/yn)yl, heterocycloalk(en)yl-Ar or acyl; or R⁷ and R^{7′} taken together with the nitrogen atom form a 5-8 membered saturated or unsaturated ring which optionally contains 1, 2 or 3 further heteroatoms; and R⁸ is hydrogen, C₁₋₆-alk(en/yn)yl, C₃₋₈-cycloalk(en)yl, C₃₋₈-cycloalk(en)yl-C₁₋₆-alk(en/yn)yl, Ar or —NR⁹R^{9′}; wherein: R⁹ and R^{9′} are each independently hydrogen, C₁₋₆-alk(en/yn)yl, C₃₋₈-cycloalk(en)yl or C₃₋₈-cycloalk(en)yl-C₁₋₆-alk(en/yn)yl; or salts thereof.

In further embodiments, R¹ is C₁₋₆-alk(en/yn)yl or a hydrogen atom.

In further embodiments, s is 0.

In further embodiments, s is 1.

In further embodiments, U is an oxygen atom.

In further embodiments, R² is hydrogen, C₁₋₆-alk(en/yn)yl, C₃₋₈-cycloalk(en)yl, Ar, Ar-C₁₋₆-alk(en/yn)yl, halogen, halo-C₁₋₆-alk(en/yn)yl or cyano; with the provisos that when R² is halogen or cyano, then s is 0; and when s is 1 and R² is a hydrogen atom, then U is O or S.

In further embodiments, Z is an oxygen atom.

In further embodiments, Z is a sulfur atom.

In further embodiments, q is 0.

In further embodiments, q is 1.

In further embodiments, X is CO.

In further embodiments, R³ is C₁₋₆-alk(en/yn)yl, C₃₋₈-cycloalk(en)yl, C₃₋₈-cycloalk(en)yl-C₁₋₆-alk(en/yn)yl, heterocycloalk(en)yl-C₁₋₆-alk(en/yn)yl, heterocycloalk(en)yl, Ar, Ar-C₁₋₆-alk(en/yn)yl, Ar-oxy-C₁₋₆-alk(en/yn)yl, Ar-C₁₋₆-alk(en/yn)yloxy-C₁₋₆-alk(en/yn)yl, C₁₋₆-alk(en/yn)yloxy-carbonyl-C₁₋₆-alk(en/yn)yl, halo-C₁-6-alk(en/yn)yl, NR¹²R^{12′}, optionally substituted NR¹²R^{12′}-C₁₋₆-alk(en/yn)yl, or optionally substituted NR¹²R^{12′}-C₃₋₈-cycloalk(en)yl.

In further embodiments, R¹² and R^{12′} are each independently hydrogen, C₁₋₆-alk(en/yn)yl or Ar.

In further embodiments, V is CH.

In further embodiments, each R⁵ is independently C₁₋₆-alk(en/yn)yl, C₁₋₆-alk(en/yn)yl-heterocycloalk(en)yl, Ar, C₁₋₆-alk(en/yn)yloxy, Ar-oxy, C₁₋₆-alk(en/yn)yloxy-carbonyl, halogen, halo-C₁₋₆-alk(en/yn)yl, NR⁷R^{7′}, S—R⁸ or SO₂R⁸; or two adjacent R⁵ groups taken together with the aromatic group form a 5-8 membered ring, which optionally contains one or two heteroatoms.

In further embodiments, both R⁷ and R^{7′} are C₁₋₆-alk(en/yn)yl.

In further embodiments, R⁸ is C₁₋₆-alk(en/yn)yl or Ar.

In further embodiments, the Kv7 channel activator is selected from the group consisting of: 2-(4-Fluorophenyl)-N-{2-methyl-4-[(6-p-tolyloxypyridin-3-ylmethyl)-amino]-phenyl}-acetamide; 2-(4-Fluorophenyl)-N-{2-methyl-4-[(6-trifluoromethylpyridin-3-ylmethyl)-amino]-phenyl}-acetamide; 3,3-Dimethyl-N-{2-methyl-4-[(6-p-tolyloxypyridin-3-ylmethyl)-amino]-phenyl}-butyramide; 3,3-Dimethyl-N-{2-methyl-4-[(6-trifluoromethylpyridin-3-ylmethyl)-amino]-phenyl}-butyramide; N-(4-{[6-(4-Cyanophenoxy)-pyridin-3-ylmethyl]-amino}-2-methylphenyl)-2-(4-fluorophenyl)-acetamide; N-{4-[(6-Chloropyridin-3-ylmethyl)-amino]-2-methylphenyl}-2-(4-fluorophenyl)-acetamide; or 2,2-Dimethyl-N-{2-methyl-4-[(6-phenoxypyridin-3-ylmethyl)-amino]-phenyl}-proprionamide; or a salt thereof.

In further embodiments, the Kv7 channel activator is a compound according to formula 11 wherein: U is O, S or NR^{2′}; s is 0 or 1; X is CO or SO₂; Z is O, S or NR⁴; wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl and hydroxy-C₃₋₈-cycloalk(en)yl; q is 0 or 1; R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; R² is selected from the group consisting of hydrogen, halogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that: when R² is halogen or cyano, then s is 0; and when s is 1 and U is NR^{2′}, then R^{2′} is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together form a 5-8 membered saturated or unsaturated ring that optionally contains one further heteroatom; R³ is selected from the group consisting of C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; and Y represents a group of the following formulas:

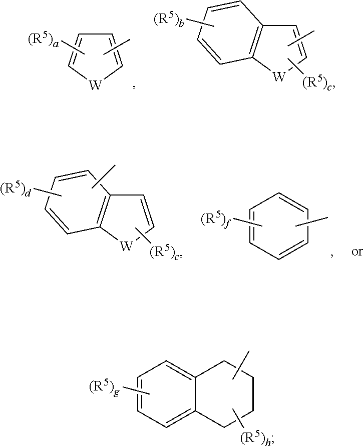

wherein: the line represents a bond attaching the group represented by Y to the nitrogen atom; W is O or S; a is 0, 1, 2 or 3; b is 0, 1, 2, 3 or 4; c is 0 or 1; d is 0, 1, 2 or 3; e is 0, 1 or 2; f is 0, 1, 2, 3, 4 or 5; g is 0, 1, 2, 3 or 4; h is 0, 1, 2 or 3; and each R⁵ is independently selected from the group consisting of a C₁₋₆-alk(en/yn)yl, C₃₋₈-cycloalk(en)yl, Ar, C₃₋₈-cycloalk(en)yl-C₁₋₆-alk(en/yn)yl, Ar-C₁₋₆-alk(en/yn)yl, acyl, C₁₋₆-alk (an/en/yn)yloxy, halogen, halo-C₁₋₆-alk(en/yn)yl, —CO—NR⁶R^{6′}, cyano, nitro, —NR⁷R^{7′}, —S—R⁸ and SO₂OR⁸; or two adjacent R⁵ substituents together with the aromatic group form a 5-8 membered saturated or unsaturated ring that optionally contains one or two heteroatoms; wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; with the provisos that; when R⁵ is SO₂OR⁸ then R⁸ is not —NR⁹R^{9′}; and when R⁵ is SO₂R⁸, then R⁸ is not a hydrogen atom; or a pharmaceutically acceptable salt thereof; with the proviso that the compound of formula I is not: N-[4-[[(4-aminophenyl)amino]methyl]phenyl]-acetamide; N-[4-[[(4-amino-2-methylphenyl)amino]methyl]phenyl]-acetamide; N-[4-[[(4-amino-3-methylphenyl)amino]methyl]phenyl]-acetamide; 2-[[[4-(acetylamino)phenyl]methyl]amino]-5-chloro-N-(5-chloro-2-pyridinyl)-benzamide; N-[4-[[(3,4,5-trimethoxyphenyl)amino]methyl]phenyl]-acetamide; N-[4-[[(5,6,7,8-tetrahydro-5,5,8,8-tetramethyl-2-naphthalenyl)amino]methyl]phenyl]-acetamide; N-[4-[[[3-(1H-imidazol-1-ylmethyl)phenyl]amino]methyl]phenyl]-acetamide; N-[4-[[[2-(1H-imidazol-1-ylmethyl)phenyl]amino]methyl]phenyl]-acetamide; N-[4-[[(4-amino-3,5-dichlorophenyl)amino]methyl]phenyl]-acetamide; N-[4-[[(2,4-diamino-6-quinazolinyl)amino]methyl]phenyl]-acetamide; or N-[4-[[(2,4-diamino-6-quinazolinyl)amino]methyl]phenyl]-acetamide.

In further embodiments, R¹ and R^{1′} are independently selected from the group consisting of hydrogen and C₁₋₆-alk(en/yn)yl.

In further embodiments, at least one of R¹ and R^{1′} is a hydrogen atom.

In further embodiments, s is 1.

In further embodiments, s is 0.

In further embodiments, R² is selected from the group consisting of hydrogen, C₁₋₆-alk(en/yn)yl, Ar and halogen, provided that when R is halogen, then s is 0.

In further embodiments, U is NR^{2′} and at least one of R and R^{2′} is a hydrogen atom. In further embodiments, both R² and R² are hydrogen atoms.

In further embodiments, X is CO.

In further embodiments, q is 0.

In further embodiments, q is 1.

In further embodiments, Z is an oxygen atom.

In further embodiments, R³ is C₁₋₆-alk(en/yn)yl.

In further embodiments, each R⁵ is independently selected from the group consisting of a C₁₋₆-alk(en yn)yl, C₃₋₈-cycloalk(en)yl, Ar, cyano, halogen, halo-Ct₋₆-alk(en/yn)yl and C₁₋₆-alk (an/en/yn)yloxy or two adjacent substituents together form a 5-8 membered saturated or unsaturated ring which optionally contains one or two heteroatoms.

In further embodiments, the Kv7 channel activator is selected from the group consisting of: {2-Amino-4-[(4-tert-butyl phenylamino)-methyl]-phenyl}-carbamic acid ethyl ester; (2-Amino-4-phenylaminomethyl-phenyl)-carbamic acid ethyl ester; [2-Amino-4-(aphthalene-2-ylaminomethyl)-phenyl]carbamic acid ethyl ester; [2-Amino-4-(p-tolylamino-methyl)-phenyl]carbamic acid ethyl ester; {2-Amino-4-[(4-trifluoromethylphenylamino)-methyl]-phenyl}-carbamic acid ethyl ester; {2-Amino-4-[(4-chlorophenylamino)-methyl]-phenyl}-carbamic acid ethyl ester; {2-Amino-4-[(3-fluorophenylamino)-methyl]-phenyl}-carbamic acid ethyl ester; {2-Amino-4-[(4-fluorophenylamino)-methyl]-phenyl}-carbamic acid ethyl ester; {2-Amino-4-[(2-fluorophenylamino)-methyl]-phenyl}-carbamic acid ethyl ester; [2-Amino-4-(biphenyl-4-ylaminomethyl)-phenyl]-carbamic acid ethyl ester; {2-Amino-4-[(2,4-difluorophenylamino)-methyl]-phenyl}-carbamic acid ethyl ester; {2-Amino-4-[(4-methoxyphenylamino)-methyl]-phenyl}-carbamic acid ethyl ester; {2-Amino-4-[(4-cyclohexylphenylamino)-methyl]-phenyl}-carbamic acid ethyl ester; [2-Amino-4-(indan-5-ylaminomethyl-phenyl]-carbamic acid ethyl ester; {2-Amino-4-[(4-isopropylphenylamino)-methyl]-phenyl}-carbamic acid ethyl ester; {2-Amino-4-[(4-butylphenylamino)-methyl]-phenyl}-carbamic acid ethyl ester; {2-Amino-4-[(4-chloro-3-fluorophenylamino)methyl]phenyl}carbamic acid ethyl ester; {2-Amino-4-[(2,4-dichlorophenylamino)methyl]phenyl}carbamic acid ethyl ester; {2-Amino-4-[(2,3-dichlorophenylamino)methyl]phenyl}carbamic acid ethyl ester; {2-Amino-4-[(3,5-dichlorophenylamino)methyl]phenyl}carbamic acid ethyl ester; {2-Amino-4-[(3,4-dichlorophenylamino)methyl]phenyl}carbamic acid ethyl ester; {2-Amino-4-[(3-trifluoromethylphenylamino)methyl]phenyl}carbamic acid ethyl ester; {2-Amino-4-[(3-fluoro-4-trifluoromethylphenylamino)methyl]phenyl}carbamic acid ethyl ester; {2-Amino-4-[(3,4-dichlorophenylamino)methyl]phenyl}carbamic acid ethyl ester; {2-Amino-4-[(4-cyanophenylamino)methyl]phenyl}carbamic acid ethyl ester; {2-Amino-4-[(4-fluoro-3-trifluoromethylphenylamino)methyl]phenyl}carbamic acid ethyl ester; {2-Amino-4-[(3-chloro-4-methylphenylamino)methyl]phenyl}carbamic acid ethyl ester; {2-Amino-4-[(3-chlorophenylamino)methyl]phenyl}carbamic acid ethyl ester; [2-Amino-4-(m-tolylaminomethyl)phenyl]carbamic acid ethyl ester; {2-Amino-4-[1-(4-chlorophenylamino)ethyl]phenyl}carbamic acid ethyl ester; {2-Amino-4-[1-(4-trifluoromethylphenylamino)ethyl]phenyl}carbamic acid ethyl ester; N-{2-Amino-4-[(3-fluorophenylamino)methyl]phenyl}-2,2-dimethylpropionamide; {4-[(4-Chlorophenylamino)methyl]phenyl}carbamic acid ethyl ester; {4-[(4-Trifluoromethylphenylamino)methyl]phenyl}carbamic acid ethyl ester; {4-[(4-Chlorophenylamino)methyl]phenyl}carbamic acid ethyl ester; {4-[(4-Fluorophenylamino)methyl]-2-methylphenyl}carbamic acid ethyl ester; {4-[(4-Chlorophenylamino)methyl]-2-methylphenyl}carbamic acid ethyl ester; {2-Methyl-4-[(4-trifluoromethylphenylamino)methyl]phenyl}carbamic acid ethyl ester; {4-[(3,4-Difluorophenylamino)methyl]-2-methylphenyl}carbamic acid ethyl ester; {4-[(3-Fluorophenylamino)methyl]-2-methylphenyl}carbamic acid ethyl ester; {2-Chloro-4-[(4-chlorophenylamino)methyl]phenyl}carbamic acid ethyl ester; {2-Chloro-4-[(4-trifluoromethyl-phenylamino)-methyl]phenyl}-carbamic acid ethyl ester; {2-Chloro-4-[(4-fluorophenylamino)methyl]phenyl}-carbamic acid ethyl ester; {2-Chloro-4-[(3-fluorophenylamino)methyl]phenyl}carbamic acid ethyl ester; {2-Chloro-4-[(3,4-dichlorophenylamino)methyl]phenyl}carbamic acid ethyl ester; {2-Chloro-4-[(4-chloro-3-fluorophenylamino)methyl]phenyl}carbamic acid ethyl ester; {4-[(4-Chlorophenylamino)methyl]-2-fluorophenyl}carbamic acid ethyl ester; {4-[(4-Chloro-3-fluorophenylamino)methyl]-2-fluorophenyl}carbamic acid ethyl ester; {2-Fluoro-4-[(4-trifluoromethylphenylamino)methyl]phenyl}carbamic acid ethyl ester; {4′-Dimethylamino-5-[(3-fluorophenylamino)methyl]biphenyl-2-yl}carbamic acid ethyl ester; {4′-Dimethylamino-5-[(4-trifluoromethylphenylamino)methyl]biphenyl-2-yl}carbamic acid ethyl ester; {4′-Chloro-5-[(3-fluorophenylamino)methyl]biphenyl-2-yl}carbamic acid ethyl ester; {4′-Chloro-5-[(4-trifluoromethylphenylamino)methyl]biphenyl-2-yl}carbamic acid ethyl ester; N-{4-[(4-chlorophenylamino)methyl]phenyl}butyramide; N-{4-[(3,4-dichlorophenylamino)methyl]phenyl}butyramide; N-{4-[(4-chloro-3-fluorophenylamino)methyl]phenyl}butyramide; N-{4-[(4-fluoro-phenylamino)methyl]-2-methyl phenyl}butyramide; N-{4-[(3-fluorophenylamino)methyl]-2-methylphenyl}butyramide; N-{4-[(4-chlorophenylamino)methyl]-2-methylphenyl}butyramide; N-{4-[(3,4-dichlorophenylamino)methyl]-2-methylphenyl}butyramide; N-{4-[(4-chloro-3-fluorophenylamino)methyl]-2-methyl phenyl}butyramide; N-{2-chloro-4-[(4-trifluoromethylphenylamino)methyl]phenyl}butyramide; N-{2-chloro-4-[(4-fluorophenylamino)methyl]phenyl}butyramide; N-{2-chloro-4-[(3-fluorophenylamino)methyl]phenyl}butyramide; N-{2-chloro-4-[(4-chlorophenylamino)methyl]phenyl}butyramide; N-{2-chloro-4-[(3,4-dichlorophenylamino)methyl]phenyl}butyramide; N-{2-chloro-4-[(4-chloro-3-fluorophenylamino)methyl]phenyl}butyramide; N-{2-fluoro-4-[(3-fluorophenylamino)methyl]phenyl}butyramide; N-{4-[(4-chlorophenylamino)methyl]-2-fluorophenyl}butyramide; N-{2-fluoro-4-[(4-trifluoromethylphenylamino)methyl]phenyl}butyramide; N-{-4-[(3,4-dichlorophenylamino)methyl]-2-fluorophenyl}butyramide; N-{4-[(4-chloro-3-fluorophenylamino)methyl]-2-fluorophenyl}butyramide; and pharmaceutically acceptable salts thereof.

In further embodiments, the Kv7 channel activator is a compound according to formula 11 wherein: U is O, S or Cs is O or 1; X is CO or SO₂; Z is O, S or NR, wherein R⁴ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en yn)yl and hydroxy-C₃₋₈-cycloalk(en)yl; q is O or 1; R¹ and R^{1′} are independently selected from the group consisting of hydrogen, C₁₋₆-alk(en/yn)yl, C3-₈-cycloalk(en)yl, C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, halo-C₁₋₆-alk(en/yn)yl and halo-C₃₋₈-cycloalk(en)yl; R is selected from the group consisting of hydrogen, halogen, C₁₋₆-alk(en/yn)yl, C₃₋₈-cycloalk(en)yl, C₃₋₈-cycloalk(en)yl-Ct₋₆-alk(en/yn)yl, Ar, Ar-C₁₋₆-alk(en/yn)yl, Ar-C₃₋₈-cycloalk(en)yl, acyl, hydroxy-Ct₋₆-alk(en/yn)yl, hydroxy-C₃₋₈-cycloalk(en)yl, halo-C₁₋₆-alk(en/yn)yl, halo-C₃₋₈-cycloalk(en)yl and cyano; provided that when R² is halogen or cyano then s is 0; when s is 1 and U is NR^{2′} then R^{2′} is selected from the group consisting of hydrogen, C₁₋₆-alk(en yn)yl, C₋₈-cycloalk(en)yl, C₃₋₈-cycloallc (en)yl-C₁₋₆-alk(en/yn)yl, Ar, Ar-C₁₋₆-alk(en/yn)yl, Ar-C₃₋₈-cycloalk(en)yl, acyl, hydroxy-_{. 6}-alk(en/yn)yl, hydroxy-C₃₋₈-cycloalk(en)yl, halo-C₁₋₆-alk(en/yn)yl and halo-C₃₋₈-cycloalk(en)yl; or R² and R^{2′} together form a 5-8 membered saturated or unsaturated ring which optionally contains one further heteroatom; R³ is selected from the group consisting of C₁₋₆-alk(en/yn)yl, C₃₋₈-cycloalk(en)yl, C₃₋₈-cycloalk(en)yl-Ct₋₆-alk(en/yn)yl, Ar, Ar-C₁₋₆-alk(en/yn)yl, Ar-C₃₋₈-cycloalk(en)yl, hydroxy-Ct-₆-alk(en/yn)yl, hydroxy-C₃₋₈-cycloalk(en)yl, halo-Cj_{. 6}-alk(en/yn)yl and halo-C₃₋₈-cycloalk(en)yl; and Y is selected from groups according to a formula selected from the group consisting of:

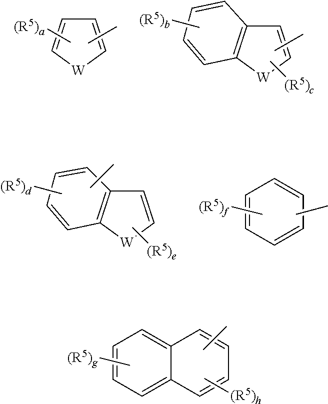

wherein: the line represents a bond attaching the group represented by Y to the nitrogen atom; W is O or S; a is 0, 1, 2 or 3; b is 0, 1, 2, 3 or 4; c is 0 or 1; d is 0, 1, 2 or 3; e is 0, 1 or 2; f is 0, 1, 2, 3, 4 or 5; g is 0, 1, 2, 3 or 4; h is 0, 1, 2 or 3; and each R⁵ is independently selected from the group consisting of a C₁₋₆-alk(en yn)yl, C₃₋₈-cycloalk(en)yl, Ar, C₃₋₈-cycloalk(en)yl-C₁₋₆-alk(en yn)yl, Ar-Ct₋₆-alk(en/yn)yl, acyl, C₁₋₆-alk (an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two substituents together form a 5-8 membered saturated or unsaturated ring which optionally contains one or two heteroatoms; R⁶ and R^{6′} are independently selected from the group consisting of hydrogen, C_{\. 6}-alk(en/yn)yl, C₃₋₈-cycloalk(en)yl, C₃₋₈-cycloalk(en)yl-C₁₋₆-alk(en/yn)yl and Ar; R⁷ and R^{7′} are independently selected from the group consisting of hydrogen, C_{\. 6}-alk(en/yn)yl, C₃₋₈-cycloalk(en)yl, C₃₋₈-cycloalk(en)yl-C₁₋₆-alk(en/yn)yl, Ar and acyl; and R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R⁹; wherein R⁹ and R^{9′} are independently selected from the group consisting of hydrogen, C₆-alk(en/yn)yl, C₃₋₈-cycloalk(en)yl and C₃₋₈-cycloalk(en)yl-C₁₋₆-alk(en/yn)yl; with the provisos that when R⁵ is SO₂OR⁸ then R⁸ is not —NR⁹R^{9′} and when R⁵ is SO₂R⁸, then R⁸ is not a hydrogen atom; or salts thereof; with the proviso that the compound of formula I is not: 2-[[[4-(acetylamino)phenyl]methyl]amino]-5-chloro-N-(5-chloro-2-pyridinyl)-benzamide; N-[4-[[(3,4,5-trimethoxyphenyl)amino]methyl]phenyl]-acetamide; N-[4-[[(5,6,7,8-tetrahydiO-5,5,8,8-tetramethyl-2-naphthalenyl)amino]methyl]phenyl]-acetamide; N-[4-[[[3-(1H-imidazol-1-ylmethyl)phenyl]amino]methyl]phenyl]-acetamide; N-[4-[[[2-(1H-imidazol-1-ylmethyl)phenyl]amino]methyl]phenyl]-acetamide; N-[4-[[[4-(IH-imidazol-1-ylmethyl)phenyl]amino]methyl]phenyl]-acetamide; N-[4-[[(4-amino-3,5-dichlorophenyl)ammo]methyl]phenyl]-acetamide; N-[4-[[(2,4-diamino-6-quinazolinyl)amino]methyl]phenyl]-acetamide; or N-[4-[[(2,4-diamino-6-quinazolinyl)amino]methyl]phenyl]-acetamide.

In further embodiments, the Kv7 channel activator is a compound according to formula 11 wherein: wherein U is O, S or NR² s is O or 1; X is CO or SO₂; Z is O, S or NR, wherein R is selected from the group consisting of hydrogen, C₁₋₆-alk(en/yn)yl, C₃₋₈-cycloalk(en)yl, C₃-s-cycloaUc(en)yl-Ct₋₆-alk(en/yn)yl, hydroxy-Ct₋₆-alk(en/yn)yl and hydroxy-C₃₋₈-cycloalk(en)yl; q is O or 1; 1 1′ R and R are independently selected from the group consisting of hydrogen, Ct_₆-alk(en/yn)yl, C₃₋₈-cycloalk(en)yl, C₃₋₈-cycloalk(en)yl-C_{1.6}-alk(en/yn)yl, acyl, hydroxy-C₁₋₆-alk(en/yn)yl, hydroxy-C₃₋₈-cycloalk(en)yl, halo-C_{1_6}-alk(en/yn)yl and halo-C_{3.8}-cycloalk(en)yl; R is selected from the group consisting of hydrogen, halogen, C₁₋₆-alk(en/yn)yl, C₃₋₈-cycloalk(en)yl, C₃₋₈-cycloalk(en)yl-C₁₋₆-alk(en yn)yl, Ar, Ar-Ct₋₆-alk(en yn)yl, Ar-C₃₋₈-cycloalk(en)yl, acyl, hydroxy-C₁₋₆-alk(en/yn)yl, hydroxy-C₃₋₈-cycloalk(en)yl, halo-Ct₋₆-alk(en/yn)yl, halo-C₃₋₈-cycloalk(en)yl and cyano; provided that when R² is halogen or cyano then s is 0; when s is 1 and U is NR² then R^{2′} is selected from the group consisting of hydrogen, C₁₋₆-alk(en/yn)yl, C₋₈-cycloalk(en)yl, C_{.8}-cycloalk(en)yl-C₁₋₆-alk(en/yn)yl, Ar, Ar-C₁₋₆-alk(en/yn)yl, Ar-C_{3.8}-cycloalk(en)yl, acyl, hydroxy-C\hat{ }₆-alk(en/yn)yl, hydroxy-C₃₋₈-cycloalk(en)yl, halo-C₁₋₆-alk(en/yn)yl and halo-C₃₋₈-cycloalk(en)yl; or R and R together form a 5-8 membered saturated or unsaturated ring which optionally contains one further heteroatom; R³ is selected from the group consisting of C₁₋₆-alk(en/yn)yl, C₃₋₈-cycloalk(en)yl, C₃₋₈-cycloalk(en)yl-C₁₋₆-alk(en yn)yl, Ar, Ar-C₁₋₆-alk(en/yn)yl, Ar-C₃₋₈-cycloalk(en)yl, hydroxy-C₁₋₆-alk(en/yn)yl, hydroxy-C₃₋₈-cycloalk(en)yl, halo-Ci-₆-alk(en yn)yl and halo-C₃-s-cycloalk(en)yl; and Y is selected from groups according to a formula selected from the group consisting of:

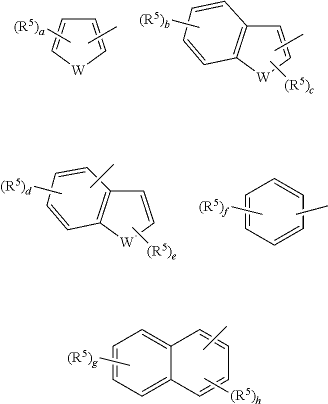

wherein, the line represents a bond attaching the group represented by Y to the nitrogen atom; W is O or S; a is 0, 1, 2 or 3; h is 0, 1, 2, 3 or 4; c is 0 or 1; d is 0, 1, 2 or 3; e is 0, 1 or 2; f is O, 1, 2, 3, 4 or 5; is 0, 1, 2, 3 or 4; h is 0, 1, 2 or 3; and each R⁵ is independently selected from the group consisting of a Ct₋₆-alk(en/yn)yl, C_{3.8}-cycloalk(en)yl, Ar, Cs-s-cycloall\hat{ }ei\hat{ }yl-Ci-e-all\hat{ }en/\hat{ }yl, Ar-Ct₋₆-alk(en/yn)yl, acyl, C₁₋₆-alk (an/en/yn)yloxy, halogen, halo-C₁₋₆-alk(en/yn)yl, —CO—NR⁶R^{6′}, cyano, nitro, —NR⁷R^{7′}, —S—R⁸, —SO₂R⁸ and SO₂OR⁸, or two substituents together form a 5-8 membered saturated or unsaturated ring which optionally contains one or two heteroatoms; R⁶ and R⁶ are independently selected from the group consisting of hydrogen, C_{\. 6}-alk(en/yn)yl, C₃₋₈-cycloalk(en)yl, C₃₋₈-cycloalk(en)yl-C₁₋₆-alk(en/yn)yl and Ar; R⁷ and R^{7′} are independently selected from the group consisting of hydrogen, Cj.₆-alk(en/yn)yl, C₃₋₈-cycloalk(en)yl, Cs-s-cycloall\hat{ }en 1- -e-all\hat{ }en/yn 1, Ar and acyl; and R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R⁹; wherein R⁹ and R^{9′} are independently selected from the group consisting of hydrogen, Q.₆-alk(en/yn)yl, C₃₋₈-cycloalk(en)yl and C₃₋₈-cycloalk(en)yl-Ct₋₆-alk(en yn)yl; with the provisos that when R⁵ is SO₂OR⁸ then R⁸ is not —NR⁹R^{9′} and when R⁵ is SO₂R⁸, then R^{s} is not a hydrogen atom; or salts thereof for increasing ion flow in a potassium channel of a mammal such as a human.

### Formula 12

In an embodiment, the Kv7 channel activator may be selected from one of the following compounds according to Formula 12. Such compounds are described in International Publication No. WO2009015667A1, published Feb. 5, 2009, and corresponding to International Application No. PCT/DK2008/050191 filed Jul. 31, 2008; International Publication No. WO2004096767A1, published Nov. 11, 2004 and corresponding to International Application No. PCT/DK2004/000283 filed Apr. 23, 2004; US Publication No. US20060264496A1 published Nov. 23, 2006 and corresponding to U.S. application Ser. No. 10/551,738 filed Apr. 23, 2003; US Publication No. US20100256145A1, published Oct. 7, 2010 and corresponding to U.S. application Ser. No. 12/671,505 filed Jul. 31, 2008; which are incorporated by reference in their entirety herein. In the case of any conflict of terminology in the context of Formula 12, these references incorporated by reference herein control.

In an embodiment, the Kv7 channel activator is a compound according to formula 12:

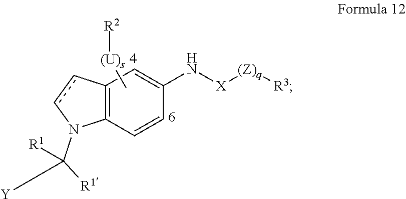

wherein, the dotted line represents an optional bond; R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹ and R^{1′} together with the carbon atom to which they are attached form a 3-8 membered saturated or unsaturated ring that optionally contains 1 or 2 heteroatoms; s is 0 or 1; U is O, NR¹¹, S, SO₂, SO₂NR¹¹, CO—O or CO—NR¹¹; wherein: R¹¹ is selected from the group consisting of hydrogen, C₁₋₆-alk(en/yn)yl, C₃₋₈-cycloalk(en)yl, and C₃₋₈-cycloalk(en)yl-C₁₋₆-alk(en/yn)yl; or R² and R^{2′} together with the nitrogen atom to which they are attached form a 4-8 membered saturated or unsaturated ring that optionally contains 1, 2 or 3 further heteroatoms; R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′-C}₁₋₆-alk(en/yn)yl, NR¹⁰R^{10′}-C₃₋₈-cycloalk(en)yl and NR¹⁰R^{10′}-C₃₋₈-cycloalk(en)yl-C₁₋₆-alk(en/yn)yl; wherein: R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C1_6-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹⁰ and R^{10′} together with the nitrogen atom to which they are attached form a 4-8 membered saturated or unsaturated ring that optionally contains 1, 2 or 3 further heteroatoms; with the proviso that; when R² is NO₂, halogen or cyano, then s is 0; and when R², R² is a hydrogen atom or acyl and s is 1, then U is NR¹¹, O or S; wherein the group -(U)ₛ-R² is linked to position 4 or 6 of the indole or indoline; q is 0 or 1; Z is O or S; X is CO or SO₂; with the proviso that when q is 0, then X is SO₂; R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar-C₁₋₆-alk(en/yn)yl, Ar-C₃₋₈-cycloalk(en)yl, Ar-heterocycloalk(en)yl, Ar-C₃₋₈-cycloalk(en)yl-C₁₋₆-alk(en/yn)yl, Ar-C₁₋₆-alk(en/yn)yl-C₃₋₈-cycloalk(en)yl, Ar-C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar-C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl, —NR¹²R^{12′}, optionally substituted NR¹²R^{12′}-C₁₋₆-alk(en/yn)yl, optionally substituted NR¹²R^{12′}—C₃₋₈-cycloalk(en)yl, and optionally substituted NR¹²R^{12′} C₃₋₈-cycloalk(en)yl-C₁₋₆-alk(en/yn)yl; wherein: R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl; or R¹² and R^{12′} together with the nitrogen atom to which they are attached form a 4-8 membered saturated or unsaturated ring that optionally contains 1, 2 or 3 further heteroatoms; with the proviso that when R³ is NR¹²R^{12′}, then q is 0; and Y represents a group of formula from the group:

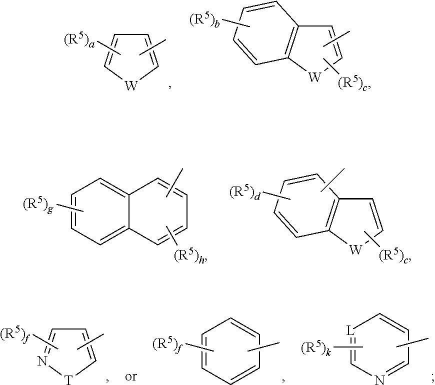

wherein: the line represents a bond attaching the group represented by Y to the carbon atom; W is O or S; T is N, NE or O; L is N, C or CH; a is 0, 1, 2 or 3; b is 0, 1, 2, 3 or 4; c is 0 or 1; d is 0, 1, 2 or 3; e is 0, 1 or 2; f is 0, 1, 2, 3, 4 or 5; g is 0, 1, 2, 3 or 4; h is 0, 1, 2 or 3; j is 0, 1, 2 or 3; with the provisos that: when T is a nitrogen atom, then j is 0, 1, 2 or 3; and when T is NH or an oxygen atom, then j is 0, 1 or 2; k is 0, 1, 2, 3 or 4; and each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸; or two adjacent R⁵ substituents together with the aromatic group to which they are attached form a 4-8 membered ring that optionally contains one or two heteroatoms; wherein: R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′}, then R⁵ is not —S—R⁸, or a pharmaceutically acceptable salt thereof. with the proviso that the compound of formula I is not: N-[1-(phenylmethyl)-1H-indol-5-yl]-Methanesulfonamide; N-[1-[(4-fluorophenyl)methyl]-1H-indol-5-yl]-Methanesulfonamide; N-[2,3-dihydro-1-(phenylmethyl)-1H-indol-5-yl]-Methanesulfonamide; N-[1-(phenylmethyl)-1H-indol-5-yl]-N′-4-quinolinyl-Urea; N-[1-(phenylmethyl)-1H-indol-5-yl]-N′-4-quinolinyl-Urea; or 1-(1-benzyl-5-indolinyl)-3-phenyl-Urea; or salts thereof.

In further embodiments, least one of R¹ or R^{1′} is a hydrogen atom.

In further embodiments, R¹ and R^{1′} are hydrogen atoms.

In further embodiments, s is 0.

In further embodiments, s is 1.

In further embodiments, R² is a hydrogen atom.

In further embodiments, R² is NO₂ or a halogen atom.

In further embodiments, U is NR¹¹.

In further embodiments, R¹¹ is a hydrogen atom.

In further embodiments, X is CO.

In further embodiments, X is SO₂.

In further embodiments, q is 0.

In further embodiments, q is 1.

In further embodiments, Z is an oxygen atom.

In further embodiments, R³ is selected from the group consisting of C₁₋₆-alk(en/yn)yl, C₃₋₈-cycloalk(en)yl, Ar, Ar-C₁₋₆-alk(en/yn)yl, Ar-oxy-C₁₋₆-alk(en/yn)yl, Ar-C₁₋₆-alk(en/yn)yloxy-C₁₋₆-alk(en/yn)yl and —NR¹²R^{12′}; with the proviso that when R³ is NR¹²R^{12′} then q is 0.

In further embodiments, R³ is NR¹²R^{12′}, q is 0 and R¹² and R^{12′} are independently selected from the group consisting of hydrogen, C₁₋₆-alk(en/yn)yl, Ar and Ar-C₁₋₆-alk(en/yn)yl, or R¹² and R^{12′} together with the nitrogen atom to which they are attached form a 4-8 membered saturated or unsaturated ring which optionally contains 1, 2 or 3 further heteroatoms.

In further embodiments, each R⁵ is independently selected from the group consisting of C₁₋₆-alk(en/yn)yl, Ar, Ar-thio, Ar-oxy, halogen and halo-C₁₋₆-alk(en/yn)yl or two adjacent R⁵ together with the aromatic group to which they are attached form a 4-8 membered ring which optionally contains one or two heteroatoms.

In further embodiments, the Kv7 channel activator is selected from the group consisting of: N-[4-Chloro-1-(4-trifluoromethylbenzyl)-2,3-dihydro-1H-indol-5-yl]-3,3-dimethylbutyramide; N-[4-Chloro-1-(5-chlorothiophen-2-ylmethyl)-2,3-dihydro-1H-indol-5-yl]-3,3-dimethylbutyramide; [1-(4-Fluorobenzyl)-2,3-dihydro-1H-indol-5-yl]-carbamic acid propyl ester; N-[1-(4-Fluorobenzyl)-2,3-dihydro-1H-indol-5-yl]-C-phenyl-methanesulfonamide; 4-Fluoro-N-[1-(4-fluorobenzyl)-2,3-dihydro-1H-indol-5-yl]-benzamide; N-[1-(4-Fluorobenzyl)-2,3-dihydro-1H-indol-5-yl]-3,3-dimethylbutyramide; N-[1-(4-Fluorobenzyl)-2,3-dihydro-1H-indol-5-yl]-2-thiophen-2-ylacetamide; N-[1-(4-Fluorobenzyl)-2,3-dihydro-1H-indol-5-yl]-2-(4-fluorophenyl)-acetamide; 3-[1-(5-Chlorothiophen-2-ylmethyl)-2,3-dihydro-1H-indol-5-yl]-1,1-diisopropylurea; Morpholine-4-carboxylic acid [1-(5-chlorothiophen-2-ylmethyl)-2,3-dihydro-1H-indol-5-yl]-amide; Pyrrolidine-1-carboxylic acid [1-(5-chlorothiophen-2-ylmethyl)-2,3-dihydro-1H-indol-5-yl]-amide; [1-(5-Chlorothiophen-2-ylmethyl)-2,3-dihydro-1H-indol-5-yl]-carbamic acid 2-benzyloxyethyl ester; 3-[1-(5-Chlorothiophen-2-ylmethyl)-2,3-dihydro-1H-indol-5-yl]-1-methyl-1-propylurea; [1-(5-Chlorothiophen-2-ylmethyl)-2,3-dihydro-1H-indol-5-yl]-carbamic acid tert-butyl ester; N-[1-(5-Chlorothiophen-2-ylmethyl)-2,3-dihydro-1H-indol-5-yl]-C-phenyl-methanesulfonamide; Butane-1-sulfonic acid [1-(5-chlorothiophen-2-ylmethyl)-2,3-dihydro-1H-indol-5-yl]-amide; N-[1-(5-Chlorothiophen-2-ylmethyl)-2,3-dihydro-1H-indol-5-yl]-4-fluorobenzamide; N-[1-(5-Chlorothiophen-2-ylmethyl)-2,3-dihydro-1H-indol-5-yl]-2,2-dimethylpropionamide; N-[1-(5-Chlorothiophen-2-ylmethyl)-2,3-dihydro-1H-indol-5-yl]-2-phenoxyacetamide; N-[1-(5-Chlorothiophen-2-ylmethyl)-2,3-dihydro-1H-indol-5-yl]-3,3-dimethylbutyramide; N-[1-(5-Chlorothiophen-2-ylmethyl)-2,3-dihydro-1H-indol-5-yl]-butyramide; Cyclopentanecarboxylic acid [1-(5-chlorothiophen-2-ylmethyl)-2,3-dihydro-1H-indol-5-yl]-amide; N-[1-(5-Chlorothiophen-2-ylmethyl)-2,3-dihydro-1H-indol-5-yl]-2-thiophen-2-ylacetamide; N-[1-(5-Chlorothiophen-2-ylmethyl)-2,3-dihydro-1H-indol-5-yl]-isonicotinamide; N-[1-(5-Chlorothiophen-2-ylmethyl)-2,3-dihydro-1H-indol-5-yl]-4-dimethylaminobenzamide; N-[1-(5-Chlorothiophen-2-ylmethyl)-2,3-dihydro-1H-indol-5-yl]-2-(4-fluorophenyl)-acetamide; N-[1-(5-Chlorothiophen-2-ylmethyl)-2,3-dihydro-1H-indol-5-yl]-6-trifluoromethylnicotinamide; 1-tert-Butyl-3-[1-(5-chlorothiophen-2-ylmethyl)-2,3-dihydro-1H-indol-5-yl]-urea; 1-[1-(5-Chlorothiophen-2-ylmethyl)-2,3-dihydro-1H-indol-5-yl]-3-ethyl urea; 1-Benzyl-3-[1-(5-chlorothiophen-2-ylmethyl)-2,3-dihydro-1H-indol-5-yl]-urea; 1-[1-(5-Chlorothiophen-2-ylmethyl)-2,3-dihydro-1H-indol-5-yl]-3-phenethyl urea; 1-[1-(5-Chlorothiophen-2-ylmethyl)-2,3-dihydro-1H-indol-5-yl]-3-thiophen-2-ylurea; 1-[1-(5-Chlorothiophen-2-ylmethyl)-2,3-dihydro-1H-indol-5-yl]-3-thiophen-3-ylurea; [1-(5-Chlorothiophen-2-ylmethyl)-2,3-dihydro-1H-indol-5-yl]-carbamic acid propyl ester; 2,2-Dimethyl-N-[6-nitro-1-(4-trifluoromethylbenzyl)-2,3-dihydro-1H-indol-5-yl]-propionamide; N-[1-(5-Chlorothiophen-2-ylmethyl)-6-nitro-2,3-dihydro-1H-indol-5-yl]-2,2-dimethylpropionamide; 2-(4-Fluorophenyl)-N-[6-nitro-1-(4-trifluoromethylbenzyl)-2,3-dihydro-1H-indol-5-yl]-acetamide; N-[1-(5-Chlorothiophen-2-ylmethyl)-6-nitro-2,3-dihydro-1H-indol-5-yl]-2-(4-fluorophenyl)-acetamide; N-[1-(5-Chlorothiophen-2-ylmethyl)-6-nitro-2,3-dihydro-1H-indol-5-yl]-3,3-dimethylbutyramide; N-[6-Amino-1-(5-chlorothiophen-2-ylmethyl)-2,3-dihydro-1H-indol-5-yl]-3,3-dimethylbutyramide; N-[6-Amino-1-(4-trifluoromethylbenzyl)-2,3-dihydro-1H-indol-5-yl]-2,2-dimethylpropionamide; N-[6-Amino-1-(5-chlorothiophen-2-ylmethyl)-2,3-dihydro-1H-indol-5-yl]-2,2-dimethylpropionamide; N-[6-Amino-1-(4-trifluoromethylbenzyl)-2,3-dihydro-1H-indol-5-yl]-2-(4-fluorophenyl)-acetamide; N-[6-Amino-1-(4-trifluoromethylbenzyl)-2,3-dihydro-1H-indol-5-yl]-3,3-dimethylbutyramide, N-[6-Amino-1-(4-fluorobenzyl)-2,3-dihydro-1H-indol-5-yl]-3,3-dimethylbutyramide; N-[6-Amino-1-(3-fluoro-4-trifluoromethylbenzyl)-2,3-dihydro-1H-indol-5-yl]-3,3-dimethylbutyramide; N-[1-(5-Chlorothiophen-2-ylmethyl)-1H-indol-5-yl]-3,3-dimethylbutyramide; N-[6-Bromo-1-(4-trifluoromethylbenzyl)-2,3-dihydro-1H-indol-5-yl]-3,3-dimethylbutyramide; N-[6-Bromo-1-(5-chlorothiophen-2-ylmethyl)-2,3-dihydro-1H-indol-5-yl]-3,3-dimethylbutyramide; N-[1-(4-Chlorobenzyl)-2,3-dihydro-1H-indol-5-yl]-3,3-dimethylbutyramide; 3,3-Dimethyl-N-[1-(4-trifluoromethylbenzyl)-2,3-dihydro-1H-indol-5-yl]-butyramide; N-[1-(4-Isopropylbenzyl)-2,3-dihydro-1H-indol-5-yl]-3,3-dimethylbutyramide; N-[1-(3-Fluoro-4-trifluoromethylbenzyl)-2,3-dihydro-1H-indol-5-yl]-3,3-dimethylbutyramide; N-[1-(6-Chlorobenzo[1,3]dioxol-5-ylmethyl)-2,3-dihydro-1H-indol-5-yl]-3,3-dimethylbutyramide, N-[1-(3,5-Dimethyl-1-phenyl-1H-pyrazol-4-ylmethyl)-2,3-dihydro-1H-indol-5-yl]-3,3-dimethylbutyramide; N-[1-(2-Chloro-5-trifluoromethylbenzyl)-2,3-dihydro-1H-indol-5-yl]-3,3-dimethylbutyramide; N-{1-[5-(4-Chlorophenoxy)-1,3-dimethyl-1H-pyrazol-4-ylmethyl]-2,3-dihydro-1H-indol-5-yl}-3,3-dimethylbutyramide; 3,3-Dimethyl-N-[1-(6-p-tolyloxy-pyridin-3-ylmethyl)-2,3-dihydro-1H-indol-5-yl]-butyramide; N-{1-[6-(4-Chlorophenyl sulfanyl)-pyridin-3-ylmethyl]-2,3-dihydro-1H-indol-5-yl}-3,3-dimethylbutyramide, N-{1-[6-(4-Cyanophenoxy)-pyridin-3-ylmethyl]-2,3-dihydro-1H-indol-5-yl}-3,3-dimethylbutyramide; 3,3-Dimethyl-N-[1-(6-trifluoromethylpyridin-3-ylmethyl)-2,3-dihydro-1H-indol-5-yl]-butyramide; 3,3-Dimethyl-N-[1-(3-methyl-benzo[b]thiophen-2-ylmethyl)-2,3-dihydro-1H-indol-5-yl]-butyramide; N-[1-(6-Fluoro-4H-benzo[1,3]dioxin-8-ylmethyl)-2,3-dihydro-1H-indol-5-yl]-3,3-dimethylbutyramide; 3,3-Dimethyl-N-[1-(6-phenoxypyridin-3-ylmethyl)-2,3-dihydro-1H-indol-5-yl]-butyramide; 3,3-Dimethyl-N-[1-(3-methyl-5-phenyl-isoxazol-4-ylmethyl)-2,3-dihydro-1H-indol-5-yl]-butyramide, N-(1-Benzo[b]thiophen-2-ylmethyl-2,3-dihydro-1H-indol-5-yl)-3,3-dimethylbutyramide; N-{1-[1-(4-Fluorophenyl)-5-methyl-1H-pyrazol-4-ylmethyl]-2,3-dihydro-1H-indol-5-yl}-3,3-dimethylbutyramide; 3,3-Dimethyl-N-[1-(5-methyl thiophen-2-ylmethyl)-2,3-dihydro-1H-indol-5-yl]-butyramide; 3,3-Dimethyl-N-[1-(4-pyrrol-1-yl-benzyl)-2,3-dihydro-1H-indol-5-yl]-butyramide; N-[1-(4-Chlorobenzyl)-2,3-dihydro-1H-indol-5-yl]-2-(4-fluorophenyl)-acetamide; 2-(4-Fluorophenyl)-N-[1-(4-trifluoromethylbenzyl)-2,3-dihydro-1H-indol-5-yl]-acetamide; 2-(4-Fluorophenyl)-N-[1-(4-isopropylbenzyl)-2,3-dihydro-1H-indol-5-yl]-acetamide; 2-(4-Fluorophenyl)-N-[1-(3-fluoro-4-trifluoromethylbenzyl)-2,3-dihydro-1H-indol-5-yl]-acetamide; N-[1-(6-Chlorobenzo[1,3]dioxol-5-ylmethyl)-2,3-dihydro-1H-indol-5-yl]-2-(4-fluorophenyl)-acetamide; N-[1-(3,5-Dimethyl-1-phenyl-1H-pyrazol-4-ylmethyl)-2,3-dihydro-1H-indol-5-yl]-2-(4-fluorophenyl)-acetamide; N-[1-(2-Chloro-5-trifluoromethylbenzyl)-2,3-dihydro-1H-indol-5-yl]-2-(4-fluorophenyl)-acetamide; N-{1-[5-(4-Chlorophenoxy)-1,3-dimethyl-1H-pyrazol-4-ylmethyl]-2,3-dihydro-1H-indol-5-yl}-2-(4-fluorophenyl)-acetamide; N-{1-[6-(4-Cyanophenoxy)-pyridin-3-ylmethyl]-2,3-dihydro-1H-indol-5-yl}-2-(4-fluorophenyl)-acetamide; 2-(4-Fluorophenyl)-N-[1-(3-methyl-benzo[b]thiophen-2-ylmethyl)-2,3-dihydro-1H-indol-5-yl]-acetamide; N-[1-(6-Fluoro-4H-benzo[1,3]dioxin-8-ylmethyl)-2,3-dihydro-1H-indol-5-yl]-2-(4-fluorophenyl)-acetamide; 2-(4-Fluorophenyl)-N-[1-(6-phenoxypyridin-3-ylmethyl)-2,3-dihydro-1H-indol-5-yl]-acetamide; N-(1-Benzo[b]thiophen-2-ylmethyl-2,3-dihydro-1H-indol-5-yl)-2-(4-fluorophenyl)-acetamide; 2-(4-Fluorophenyl)-N-{1-[1-(4-fluorophenyl)-5-methyl-1H-pyrazol-4-ylmethyl]-2,3-dihydro-1H-indol-5-yl}-acetamide; 2-(4-Fluorophenyl)-N-[1-(5-methylthiophen-2-ylmethyl)-2,3-dihydro-1H-indol-5-yl]-acetamide; 2-(4-Fluorophenyl)-N-[1-(4-pyrrol-1-yl-benzyl)-2,3-dihydro-1H-indol-5-yl]-acetamide; and pharmaceutically acceptable salts thereof.

### Formula 13

In an embodiment, the Kv7 channel activator may be selected from one of the following compounds according to Formula 13. Such compounds are described in International Publication No. WO2009015667A1, published Feb. 5, 2009, and corresponding to International Application No. PCT/DK2008/050191 filed Jul. 31, 2008; US Publication No. US20100256145A1, published Oct. 7, 2010 and corresponding to U.S. application Ser. No. 12/671,505 filed Jul. 31, 2008; which are incorporated by reference in their entirety herein. In the case of any conflict of terminology in the context of Formula 13, these references incorporated by reference herein control.

In an embodiment, the Kv7 channel activator is a compound according to formula 13.

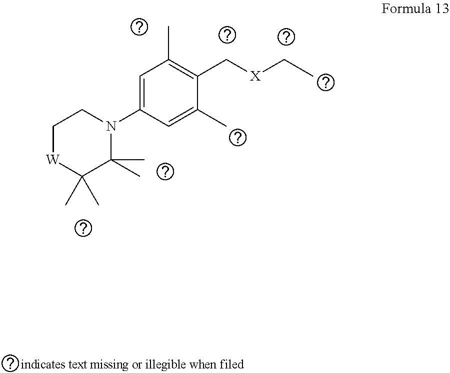

wherein: q is 0 or 1; W is O or S; X is CO; Z is O; R1 is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy; R2 is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, optionally substituted phenyl and optionally substituted pyridyl; wherein the phenyl and pyridyl are optionally substituted with one or more substituents independently being halogen, C₁₋₆-alk(en/yr) yl, C₃₋₈-cycloalk(en)yl or C₃₋₈-cycloalk(en)yl-C₁₋₆-alk(en/yn)yl; R3 is selected from the group consisting of C₁₋₁₀-alk(en/yn)yl, C₃₋₈-cycloalk(en)yl, C₃₋₈-cycloalk(en)yl-C₁₋₆-alk(en/yn)yl,

Ar-C₁₋₆-alk(en/yn)yl, Ar-C₃₋₈-cycloalk(en)yl, Ar-C₃₋₈-cycloalk(en)yl-C₁₋₆-alk(en/yn)yl and Ar; and each of R4, R5, R6 and R7 is independently selected from the group consisting of hydrogen and Ar; as the free base or a salt thereof.

In further embodiments, the Kv7 channel activator is selected from the group consisting of: N-(2-Bromo-4-morpholin-4-yl-6-trifluoromethyl-phenyl)-2-(4-fluoro-phenyl)-acetamide; 2-Cyclopentyl-N-(2-bromo-6-trifluoromethyl-4-morpholin-4-yl-phenyl)-acetamide; N-(2-Bromo-4-morpholin-4-yl-6-trifluoromethyl-phenyl)-3-cyclopentyl-propionamide; N-(2-Chloro-6-cyano-4-morpholin-4-yl-phenyl)-3-cyclohexyl-propionamide; 2-Cyclopentyl-N-(2,6-dimethyl-4-thiomorpholin-4-yl-phenyl)-acetamide; 2-Cyclopentyl-N-[2,6-dimethyl-4-(2-phenyl-morpholin-4-yl)-phenyl]-acetamide; 2-Cyclopentyl-N-[2,6-dimethyl-4-(2-phenyl-thiomorpholin-4-yl)-phenyl]-acetamide; 2-Cyclopentyl-N-[2,6-dimethyl-4-(3-pyridin-3-yl-thiomorpholin-4-yl)-phenyl]-acetamide; 2-Cyclopentyl-N-{2,6-dimethyl-4-[2-(4-trifluoromethyl-phenyl)-thiomorpholin-4-yl]-phenyl}-acetamide; N-{4-[2-(2-Chloro-phenyl)-thiomorpholin-4-yl]-2,6-dimethyl-phenyl}-2-cyclopentyl-acetamide; 2-Bicyclo[2.2.1]hept-2-yl-N-(2,6-dimethyl-4-morpholin-4-yl-phenyl)-acetamide; 2-Cyclohexyl-N-(2,6-dimethyl-4-morpholin-4-yl-phenyl)-acetamide; 3-(3,4-Difluoro-phenyl)-N-(2,6-dimethyl-4-morpholin-4-yl-phenyl)-propionamide; 2-Cyclopentyl-N-(2,6-dimethyl-4-morpholin-4-yl-phenyl)-acetamide; (2,6-Dimethyl-4-morpholin-4-yl-phenyl)-carbamic acid butyl ester; 2-(4-Chloro-phenyl)-N-(2,6-dimethyl-4-morpholin-4-yl-phenyl)-acetamide; 2,3-Dihydro-benzofuran-2-carboxylic acid (2,6-dimethyl-4-morpholin-4-yl-phenyl)-amide; 3-Cyclohexyl-N-(2,6-dimethyl-4-morpholin-4-yl-phenyl)-propionamide; 3-Cyclopentyl-N-(2,6-dimethyl-4-morpholin-4-yl-phenyl)-propionamide; N-(2,6-Dimethyl-4-morpholin-4-yl-phenyl)-2-(4-fluoro-phenyl)-acetamide; N-(2,6-Dimethyl-4-morpholin-4-yl-phenyl)-2-thiophen-2-yl-acetamide; N-(2,6-Dimethyl-4-morpholin-4-yl-phenyl)-3,3-dimethyl-butyramide; Hexanoic acid (2,6-dimethyl-4-morpholin-4-yl-phenyl)-amide; 2-Cycloheptyl-N-(2,6-dimethyl-4-morpholin-4-yl-phenyl)-acetamide; (2,6-Dimethyl-4-morpholin-4-yl-phenyl)-carbamic acid benzyl ester; (2,6-Dimethyl-4-morpholin-4-yl-phenyl)-carbamic acid 2-chloro-benzyl ester; 3,5,5-Trimethyl-hexanoic acid (2,6-dimethyl-4-morpholin-4-yl-phenyl)-amide; Octanoic acid (2,6-dimethyl-4-morpholin-4-yl-phenyl)-amide; Heptanoic acid (2,6-dimethyl-4-morpholin-4-yl-phenyl)-amide; N-(2,6-Dimethyl-4-morpholin-4-yl-phenyl)-2-phenyl-acetamide; 2-(3,4-Dichloro-phenyl)-N-(2,6-dimethyl-4-morpholin-4-yl-phenyl)-acetamide; 2-(4-Allyloxy-3-chloro-phenyl)-N-(2,6-dimethyl-4-morpholin-4-yl-phenyl)-acetamide; N-(2,6-Dimethyl-4-morpholin-4-yl-phenyl)-2-(3-trifluoromethyl-phenyl)-acetamide; N-(2,6-Dimethyl-4-morpholin-4-yl-phenyl)-2-naphthalen-2-yl-acetamide; 3-(3-Chloro-phenyl)-N-(2,6-dimethyl-4-morpholin-4-yl-phenyl)-propionamide; N-(2,6-Dimethyl-4-morpholin-4-yl-phenyl)-2-(3,4-dimethyl-phenyl)-acetamide; 2-(3-Bromo-phenyl)-N-(2,6-dimethyl-4-morpholin-4-yl-phenyl)-acetamide; 2-(3-Chloro-phenyl)-N-(2,6-dimethyl-4-morpholin-4-yl-phenyl)-acetamide; N-(2,6-Dimethyl-4-morpholin-4-yl-phenyl)-2-p-tolyl-acetamide; N-(2,6-Dimethyl-4-morpholin-4-yl-phenyl)-2-m-tolyl-acetamide; 2-(3,4-Difluoro-phenyl)-N-(2,6-dimethyl-4-morpholin-4-yl-phenyl)-acetamide; N-(2,6-Dimethyl-4-morpholin-4-yl-phenyl)-2-(3-fluoro-phenyl)-acetamide; N-(2-Bromo-4-morpholin-4-yl-6-trifluoromethyl-phenyl)-3-cyclohexyl-propionamide; N-(2-Bromo-4-morpholin-4-yl-6-trifluoromethyl-phenyl)-2-(3-fluoro-phenyl)-acetamide; N-(2-Bromo-4-morpholin-4-yl-6-trifluoromethyl-phenyl)-propionamide; N-(2-Bromo-4-morpholin-4-yl-6-trifluoromethyl-phenyl)-butyramide; N-(2-Chloro-4-morpholin-4-yl-6-trifluoromethyl-phenyl)-2-(3-fluoro-phenyl)-acetamide; N-(2-Chloro-4-morpholin-4-yl-6-trifluoromethyl-phenyl)-2-cyclopentyl-acetamide; 2-Cyclopentyl-N-{2,6-dimethyl-4-[2-(4-trifluoromethyl-phenyl)-morpholin-4-yl]-phenyl}-acetamide; N-{4-[2-(2-Chloro-phenyl)-morpholin-4-yl]-2,6-dimethyl-phenyl}-2-cyclopentyl-acetamide; 2-Cyclopentyl-N-{4-[2-(4-fluoro-phenyl)-morpholin-4-yl]-2,6-dimethyl-phenyl}-acetamide; 2-(2-Chloro-phenyl)-N-(2,6-dimethyl-4-morpholin-4-yl-phenyl)-acetamide; Pentanoic acid (2,6-dimethyl-4-morpholin-4-yl-phenyl)-amide; 4-Methyl-pentanoic acid (2,6-dimethyl-4-morpholin-4-yl-phenyl)-amide; 2-Cyclopent-2-enyl-N-(2,6-dimethyl-4-morpholin-4-yl-phenyl)-acetamide; 5-Methyl-hexanoic acid (2,6-dimethyl-4-morpholin-4-yl-phenyl)-amide; 3-Methyl-pentanoic acid (2,6-dimethyl-4-morpholin-4-yl-phenyl)-amide; Hex-5-enoic acid (2,6-dimethyl-4-morpholin-4-yl-phenyl)-amide; 3-Ethyl-pentanoic acid (2,6-dimethyl-4-morpholin-4-yl-phenyl)-amide; 2-Cyclopentyl-N-(4-morpholin-4-yl-2-pyridin-3-yl-6-trifluoromethyl-phenyl)-acetamide; 2-Cyclopentyl-N-(5-morpholin-4-yl-3-trifluoromethyl-biphenyl-2-yl)-acetamide; 2-Cyclopentyl-N-(4′-fluoro-5-morpholin-4-yl-3-trifluoromethyl-biphenyl-2-yl)-acetamide; 2-Cyclopentyl-N-(4′-methyl-5-morpholin-4-yl-3-trifluoromethyl-biphenyl-2-yl)-acetamide, 2-Cyclopentyl-N-(3′-methyl-5-morpholin-4-yl-3-trifluoromethyl-biphenyl-2-yl)-acetamide; 2-Cyclopentyl-N-(3′,4′-difluoro-5-morpholin-4-yl-3-trifluoromethyl-biphenyl-2-yl)-acetamide; 2-(4-Fluoro-phenyl)-N-(4-morpholin-4-yl-2-pyridin-3-yl-6-trifluoromethyl-phenyl)-acetamide; 2-Cyclopentyl-N-(2,6-diethyl-4-morpholin-4-yl-phenyl)-acetamide; 2-Cyclopentyl-N-(2,6-diisopropyl-4-morpholin-4-yl-phenyl)-acetamide; 2-Cyclopentyl-N-(2,6-difluoro-4-morpholin-4-yl-phenyl)-acetamide; Hexanoic acid (2,6-difluoro-4-morpholin-4-yl-phenyl)-amide; N-(2,6-Difluoro-4-morpholin-4-yl-phenyl)-3,3-dimethyl-butyramide; N-(2,6-Difluoro-4-morpholin-4-yl-phenyl)-2-(3-fluoro-phenyl)-acetamide; 2-Cyclopent-2-enyl-N-(2,6-difluoro-4-morpholin-4-yl-phenyl)-acetamide; 2-Bicyclo[2.2.1]hept-2-yl-N-(2,6-difluoro-4-morpholin-4-yl-phenyl)-acetamide; 2-Bicyclo[2.2.1]hept-2-yl-N-(2-methyl-4-morpholin-4-yl-6-trifluoromethyl-phenyl)-acetamide; 5-Methyl-pentanoic acid (2-methyl-4-morpholin-4-yl-6-trifluoromethyl-phenyl)-amide; 5-Methyl-hexanoic acid (2-methyl-4-morpholin-4-yl-6-trifluoromethyl-phenyl)-amide; 2-Cyclopent-2-enyl-N-(2-methyl-4-morpholin-4-yl-6-trifluoromethyl-phenyl)-acetamide; 2-Cyclopentyl-N-(2-methyl-4-morpholin-4-yl-6-trifluoromethyl-phenyl)-acetamide; Hexanoic acid (2-methyl-4-morpholin-4-yl-6-trifluoromethyl-phenyl)-amide; 3,3-Dimethyl-N-(2-methyl-4-morpholin-4-yl-6-trifluoromethyl-phenyl)-butyramide; 2-(3,4-Difluoro-phenyl)-N-(2-methyl-4-morpholin-4-yl-6-trifluoromethyl-phenyl)-acetamide; Hexanoic acid (2-methoxy-6-methyl-4-morpholin-4-yl-phenyl)-amide; 2-Cyclopentyl-N-(2-methoxy-6-methyl-4-morpholin-4-yl-phenyl)-acetamide; N-(2-Methoxy-6-methyl-4-morpholin-4-yl-phenyl)-3,3-dimethyl-butyramide; 2-(3,4-Difluoro-phenyl)-N-(2-methoxy-6-methyl-4-morpholin-4-yl-phenyl)-acetamide; 2-Cyclopent-2-enyl-N-(2-methoxy-6-methyl-4-morpholin-4-yl-phenyl)-acetamide; 2-(3-Fluoro-phenyl)-N-(2-methoxy-6-methyl-4-morpholin-4-yl-phenyl)-acetamide; 2-Bicyclo[2.2.1]hept-2-yl-N-(2-methoxy-6-methyl-4-morpholin-4-yl-phenyl)-acetamide; 4-Methyl-pentanoic acid (2-methoxy-6-methyl-4-morpholin-4-yl-phenyl)-amide; 5-Methyl-Hexanoic acid (2-methoxy-6-methyl-4-morpholin-4-yl-phenyl)-amide; N-(2-Chloro-6-methyl-4-morpholin-4-yl-phenyl)-2-(3-fluoro-phenyl)-acetamide; and N-(2-Chloro-6-methyl-4-morpholin-4-yl-phenyl)-2-cyclopentyl-acetamide; as the free base or a pharmaceutically acceptable salt thereof.

### Formula 14

In an embodiment, the Kv7 channel activator may be selected from one of the following compounds according to Formula 14. Such compounds are described in International Publication No. WO2009015667A1, published Feb. 5, 2008 and corresponding to International Application No. PCT/DK2008/050191 filed Jul. 31, 2008; International Publication No. WO2006029623 A1, published Mar. 23, 2006 and corresponding to International Application No. PCT/DK2005/000560 filed Sep. 2, 2005; U.S. Pat. No. 7,601,870, issued Oct. 13, 2009 and corresponding to U.S. application Ser. No. 11/312,664 filed Dec. 20, 2005; US Publication No.

US20100256145A1, published Oct. 7, 2010 and corresponding to U.S. application Ser. No. 12/671,505 filed Jul. 31, 2008; which are incorporated by reference in their entirety herein.

In the case of any conflict of terminology in the context of Formula 14, these references incorporated by reference herein control.

In an embodiment, the Kv7 channel activator is a compound according to formula 14:

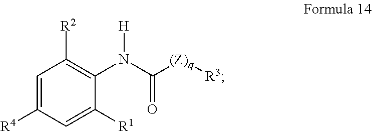

wherein, Z is O or S; q is 0 or 1; each of R¹ and R² is independently selected from the group consisting of halogen, cyano, amino, C₁₋₆-alk(en/yn)yl, C₃₋₈-cycloalk(en)yl, C₃₋₈-cycloalk(en)yl-C₁₋₆-alk(en/yn)yl, C₃₋₈-heterocycloalk(en)yl, Aryl, Heteroaryl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, C₃₋₈-cycloalk(en)yl-C₁₋₆-alk(en/yn)yloxy, and C₃₋₈-heterocycloalk(en)yloxy; R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₃₋₈-heterocycloalk(en)yl-C₁₋₆-alk(en/yn)yl, C₁₋₆-alk(en/yn)yl-C₃₋₈-heterocycloalk(en)yl-C₁₋₆-alk(en/yn)yl, Heteroaryl-C alk(en/yn)yl, Heteroaryl-C₃₋₈-cycloalk(en)yl, Heteroaryl-C₃₋₈-cycloalk(en)yl-C₁₋₆-alk(en/yn)yl, amino-C₁₋₆-alk(en/yn)yl, amino-C₃₋₈-cycloalk(en)yl, amino-C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; and R⁴ is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, C₃₋₈-heterocycloalk(en)yl, Aryl, Heteroaryl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, Aryl-C₃₋₈-heterocycloalk(en)yl, halo-C₁₋₆-alk(en/yn)yl, halo-C₃₋₈-cycloalk(en)yl, halo-C₃₋₈-cycloalk(en)yl-C₁₋₆-alk(en/yn)yl, halo-C₁₋₆-alk(en/yn)yl-C₃₋₈-heterocycloalk(en)yl-C₁₋₆-alk(en/yn)yl, NR⁵R⁶ and R⁷NH—C₁₋₆-alk(en/yn)yl; wherein: R⁵ and R⁶ are independently selected from the group consisting of hydrogen, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₁₋₆-alk(en/yn)yl, C₃₋₈-cycloalk(en)yl, C₃₋₈-cycloalk(en)yl-C₁₋₆-alk(en/yn)yl, Heteroaryl-C₁₋₆-alk(en/yn)yl, Heteroaryl-C₃₋₈-cycloalk(en)yl and Heteroaryl-C₃₋₈-cycloalk(en)yl-C₁₋₆-alk(en/yn)yl, with the proviso that R⁵ and R⁶ are not hydrogen at the same time; and R⁷ is selected from the group consisting of C₁₋₆-alk(en/yn)yl, C₃₋₈-cycloalk(en)yl, C₃₋₈-cycloalk(en)yl-C₁₋₆-alk(en/yn)yl,
Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, Aryl-C₁₋₆-alk(en/yn)yl, Aryl-C₃₋₈-cycloalk(en)yl and Heteroaryl; or a pharmaceutically acceptable salt thereof.

In further embodiments, the Kv7 channel activator is selected from the group consisting of: Hexanoic acid (4-bromo-2,6-dimethyl-phenyl)-amide; N-(4-Bromo-2,6-dimethyl-phenyl)-2-(4-fluoro-phenyl)-acetamide; N-(2-Bromo-4,6-dimethyl-phenyl)-2-(4-fluoro-phenyl)-acetamide; N-(2-Bromo-4,6-dimethyl-phenyl)-3,3-dimethyl-butyramide; N-(2-Bromo-4,6-dimethyl-phenyl)-2-cyclopentyl-acetamide; N-(2-Bromo-4,6-dichloro-phenyl)-3,3-dimethyl-butyramide; N-(2-Bromo-4,6-dichloro-phenyl)-2-(4-fluoro-phenyl)-acetamide; N-(2-Bromo-4,6-dichloro-phenyl)-2-cyclopentyl-acetamide; Heptanoic acid (4-bromo-2,6-dimethyl-phenyl)-amide; Cyclohexanecarboxylic acid (4-bromo-2,6-dimethyl-phenyl)-amide; N-(4-Bromo-2,6-dimethyl-phenyl)-2-thiophen-2-yl-acetamide; 2-Phenyl-cyclopropanecarboxylic acid (4-bromo-2,6-dimethyl-phenyl)-amide; N-(4-Bromo-2,6-dimethyl-phenyl)-2-(4-chloro-phenyl)-acetamide; Pentanoic acid (4-bromo-2,6-dimethyl-phenyl)-amide; Octanoic acid (4-bromo-2,6-dimethyl-phenyl)-amide; N-(4-Bromo-2,6-dimethyl-phenyl)-2-cyclopentyl-acetamide; 2-Bicyclo[2.2.1]kept-2-yl-N-(2,4-difluoro-6-morpholin-4-yl-phenyl)-acetamide; (S)-2-Amino-N-{2,6-dimethyl-4-[methyl-(4-trifluoromethyl-benzyl)-amino]-phenyl}-3-methyl-butyramide; (S)-2-Amino-4-methyl-pentanoic acid {2,6-dimethyl-4-[methyl-(4-trifluoromethyl-benzyl)-amino]-phenyl}-amide;

(4-Bromo-2,6-dimethyl-phenyl)-carbamic acid ethyl ester; (4-Bromo-2,6-dimethyl-phenyl)-carbamic acid propyl ester; N-(2-Amino-4-bromo-6-methyl-phenyl)-3,3-dimethyl-butyramide; 2-Cyclopentyl-N-{2,6-dimethyl-4-[2-(4-trifluoromethyl-phenyl)-pyrrolidin-1-yl]-phenyl}-acetamide; N-(4-Azepan-1-yl-2,6-dimethyl-phenyl)-2-cyclopentyl-acetamide; 2-Cyclopentyl-N-(2,6-dimethyl-4-pyrrol-1-yl-phenyl)-acetamide; N-(3′-Amino-3,5-dimethyl-biphenyl-2-yl)-2-(4-fluoro-phenyl)-acetamide; N-(4′-Dimethylamino-3,5-dimethyl-biphenyl-2-yl)-2-(4-fluoro-phenyl)-acetamide; N-(2,4-Dimethyl-6-quinolin-3-yl-phenyl)-2-(4-fluoro-phenyl)-acetamide; 2-(4-Fluoro-phenyl)-N-(4′-hydroxy-3′-methoxy-3,5-dimethyl-biphenyl-2-yl)-acetamide; 2-(4-Fluoro-phenyl)-N-(3′-hydroxy-3,5-dimethyl-biphenyl-2-yl)-acetamide; 2-(4-Fluoro-phenyl)-N-(2′-methanesulfonylamino-3,5-dimethyl-biphenyl-2-yl)-acetamide; N-(4′-Isopropyl-3,5-dimethyl-biphenyl-2-yl)-3,3-dimethyl-butyramide; 2-Cyclopentyl-N-(3,5-dimethyl-biphenyl-2-yl)-acetamide; N-(4′-Fluoro-3,5-dimethyl-biphenyl-2-yl)-2-(4-fluoro-phenyl)-acetamide; N-(3,5-Dimethyl-3′,5′-bis-trifluoromethyl-biphenyl-2-yl)-2-(4-fluoro-phenyl)-acetamide; N-(3′-Acetylamino-3,5-dimethyl-biphenyl-2-yl)-2-(4-fluoro-phenyl)-acetamide; 2-(4-Fluoro-phenyl)-N-(2′-methoxy-3,5-dimethyl-biphenyl-2-yl)-acetamide; N-(3,5-Dimethyl-4′-vinyl-biphenyl-2-yl)-2-(4-fluoro-phenyl)-acetamide; N-(3′-Cyano-3,5-dimethyl-biphenyl-2-yl)-2-(4-fluoro-phenyl)-acetamide; N-(3,5-Dimethyl-3′-trifluoromethoxy-biphenyl-2-yl)-2-(4-fluoro-phenyl)-acetamide; N-[2-(2,3-Dihydro-benzo[1,4]dioxin-6-yl)-4,6-dimethyl-phenyl]-2-(4-fluoro-phenyl)-acetamide; N-[2,4-Dimethyl-6-(2,2,5-trimethyl-2,3-dihydro-benzofuran-7-yl)-phenyl]-2-(4-fluoro-phenyl)-acetamide; N-[2,6-Dimethyl-4-(4-trifluoromethyl-benzylamino)-phenyl]-acetamide; N-{2,6-Dimethyl-4-[methyl-(4-trifluoromethyl-benzyl)-amino]-phenyl}-acetamide; {4-[(5-Chloro-thiophen-2-ylmethyl)-amino]-2,6-dimethyl-phenyl}-carbamic acid propyl ester; [4-(4-Fluoro-benzylamino)-2,6-dimethyl-phenyl]-carbamic acid propyl ester; [2,6-Dimethyl-4-(4-trifluoromethyl-benzylamino)-phenyl]-carbamic acid propyl ester; [4-(3-Fluoro-4-trifluoromethyl-benzylamino)-2,6-dimethyl-phenyl]-carbamic acid propyl ester; {2,6-Dimethyl-4-[(4-methyl-2-phenyl-pyrimidin-5-ylmethyl)-amino]-phenyl}-carbamic acid propyl ester; {2,6-Dimethyl-4-[(6-p-tolyloxy-pyridin-3-ylmethyl)-amino]-phenyl}-carbamic acid propyl ester; {4-[(6-Methoxy-pyridin-3-ylmethyl)-amino]-2,6-dimethyl-phenyl}-carbamic acid propyl ester; {4-[(3-Fluoro-4-trifluoromethyl-benzyl)-methyl-amino]-2,6-dimethyl-phenyl}-carbamic acid propyl ester; 2-Cyclopentyl-N-[2,6-dimethyl-4-(4-trifluoromethyl-benzylamino)-phenyl]-acetamide 2-Cyclopentyl-N-{2,6-dimethyl-4-[methyl-(4-trifluoromethyl-benzyl)-amino]-phenyl}-acetamide; 2-Cyclopentyl-N-{2,6-dimethyl-4-[(6-trifluoromethyl-pyridin-3-ylmethyl)-amino]-phenyl}-acetamide; N-{2,6-Dimethyl-4-[(6-trifluoromethyl-pyridin-3-ylmethyl)-amino]-phenyl}-3,3-dimethyl-butyramide; N-{2-Bromo-4-[(5-chloro-thiophen-2-ylmethyl)-amino]-6-trifluoromethyl-phenyl}-3-cyclohexyl-propionamide; {4-[(3-Fluoro-phenylamino)-methyl]-2,6-dimethyl-phenyl}-carbamic acid ethyl ester; {2,6-Dimethyl-4-[(4-trifluoromethyl-phenylamino)-methyl]-phenyl}-carbamic acid ethyl ester; 2-Cyclopentyl-N-{4-[(3-fluoro-phenylamino)-methyl]-2,6-dimethyl-phenyl}-acetamide; N-{4-[(3-Chloro-phenylamino)-methyl]-2,6-dimethyl-phenyl}-2-cyclopentyl-acetamide; 2-Cyclopentyl-N-{4-[(3-methoxy-phenylamino)-methyl]-2,6-dimethyl-phenyl}-acetamide; N-{4-[(4-Chloro-phenylamino)-methyl]-2,6-dimethyl-phenyl}-2-cyclopentyl-acetamide; 2-Cyclopentyl-N-{4-[(3,4-difluoro-phenylamino)-methyl]-2,6-dimethyl-phenyl}-acetamide; 2-Cyclopentyl-N-{2,6-dimethyl-4-[(4-trifluoromethyl-phenylamino)-methyl]-phenyl}-acetamide; 2-Cyclopentyl-N-[2,6-dimethyl-4-(p-tolylamino-methyl)-phenyl]-acetamide; 2-Cyclopentyl-N-{2,6-dimethyl-4-[(3-trifluoromethyl-phenylamino)-methyl]-phenyl}-acetamide; 2-Cyclopentyl-N-{-4-[(3,5-difluoro-phenylamino)-methyl]-2,6-dimethyl-phenyl}-acetamide; {4-[(4-Fluoro-phenylamino)-methyl]-2,6-dimethyl-phenyl}-carbamic acid propyl ester; {4-[(4-Chloro-phenylamino)-methyl]-2,6-dimethyl-phenyl}-carbamic acid propyl ester; {2,6-Dimethyl-4-[(4-trifluoromethyl-phenylamino)-methyl]-phenyl}-carbamic acid propyl ester; {4-[(3,5-Difluoro-phenylamino)-methyl]-2,6-dimethyl-phenyl}-carbamic acid propyl ester; {4-[(3-Fluoro-phenylamino)-methyl]-2,6-dimethyl-phenyl}-carbamic acid propyl ester; N-(4-Bromo-2-methyl-6-morpholin-4-yl-phenyl)-3,3-dimethyl-butyramide; {4-[(4-Methoxyphenylamino)-methyl]-2,6-dimethylphenyl}-carbamic acid propyl ester; (R)-2-Amino-4-methylpentanoic acid [2,6-dimethyl-4-(4-trifluoromethylbenzylamino)-phenyl]-amide; Pentanoic acid (4-[(4-chlorophenylamino)-methyl]-2,6-dimethylphenyl)-amide; 2-(4-Chlorophenyl)-N-[4-(4-chlorophenylamino)-methyl]-2,6-dimethyl phenyl)-acetamide; {2,6-Dimethyl-4-[(4-trifluoromethylphenylamino)-methyl]-phenyl}-carbamic acid 2-methoxyethyl ester; N-{4-[(5-Chloro-pyridin-2-ylamino)-methyl]-2,6-dimethylphenyl}-2-cyclopentylacetamide; 2-Cyclopentyl-N-{4-[(2,6-dichloro-pyridin-4-ylamino)-methyl]-2,6-dimethylphenyl}-acetamide; N-{2-Chloro-6-methyl-4-[(6-trifluoromethyl-pyridin-3-ylmethyl)-amino]-phenyl}-2-(3-fluoro-phenyl)-acetamide; N-[2-Chloro-6-trifluoromethyl-4-(4-trifluoromethylbenzylamino)-phenyl]-2-cyclopentylacetamide; [2-Amino-6-methyl-4-(4-trifluoromethylbenzylamino)-phenyl]-carbamic acid ethyl ester; 3,3-Dimethyl-N-{2-methyl-6-morpholin-4-yl-4-(4-trifluoromethylbenzylamino)-phenyl}-butyramide; 2-Cyclopentyl-N-{2,6-dichloro-4-[(4-fluoro-phenylamino)-methyl]-phenyl}-acetamide; 2-Cyclopentyl-N-{2,6-dichloro-4-[(5-trifluoromethylpyridin-2-ylamino)-methyl]-phenyl}-acetamide; and pharmaceutically acceptable salts thereof.

In further embodiments, Z is O or S; q is 0; R¹ and R² are each independently selected from the group consisting of halogen, cyano, amino, C₁₋₆-alk(en/yn)yl, C₃₋₈-cycloalk(en)yl, C₃₋₈-cycloalk(en)yl-C₁₋₆-alk(en/yn)yl, Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, and C₃₋₈-cycloalk(en)yl-C₁₋₆-alk(en/yn)yloxy; R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, amino-C₁₋₆-alk(en/yn)yl, amino-C₃₋₈-cycloalk(en)yl, amino-C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; and R⁴ is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, Aryl, Aryl-C₃₋₈-cycloalk(en)yl, Aryl-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, NR⁵R⁶ and R⁷NH—C₁₋₆-alk(en/yn)yl; wherein: R⁵ and R⁶ are each independently selected from the group consisting of hydrogen, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₁₋₆-alk(en/yn)yl, C₃₋₈-cycloalk(en)yl, and C₃₋₈-cycloalk(en)yl-C₁₋₆alk(en/yn)yl, with the proviso that R⁵ and R⁶ can not both be hydrogen; and R⁷ is selected from the group consisting of C₁₋₆-alk(en/yn)yl; C₃₋₈-cycloalk(en)yl, C₃₋₈-cycloalk(en)yl-C₁₋₆-alk(en/yn)yl, Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, Aryl-C₁₋₆-alk(en/yn)yl, and Aryl-C₃₋₈-cycloalk(en)yl; with the proviso that the compound of formula I is not N-(4-Bromo-2,6-dimethyl-phenyl)-2-cyclopentyl-acetamide; or a pharmaceutically acceptable salt thereof.

In further embodiments, R¹ and R² are each independently selected from the group consisting of halogen, amino, C₁₋₆-alk(en/yn)yl, Aryl, and halo-C₁₋₆-alk(en/yn)yl.

In further embodiments, R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, and amino-C₁₋₆-alk(en/yn)yl.

In further embodiments, R⁴ is selected from the group consisting of halogen, C₁₋₆-alk(en/yn)yl, NR⁵R⁶ and R⁷NH—C₁₋₆-alk(en/yn)yl, wherein R⁵, R⁶ and R⁷ are as previously defined.

In further embodiments, R⁴ is NR⁵R⁶, wherein R⁵ and R⁶ are independently selected from the group consisting of hydrogen, Aryl-C₁₋₆-alk(en/yn)yl, and C₁₋₆-alk(en/yn)yl, with the proviso that R⁵ and R⁶ cannot both be hydrogen.

In further embodiments, R⁴ is R⁷NH—C₁₋₆-alk(en/yn)yl, wherein R⁷ is Aryl.

In further embodiments, any Aryl is optionally substituted with one or more substituents independently selected from the group consisting of amino, halogen, cyano, C₁₋₆-alk(en/yn)yl, halo-C₁₋₆-alk(en/yn)yl, hydroxy, C₁₋₆-alk(en/yn)yloxy, halo-C₁₋₆-alk(en/yn)yloxy, di-(C₁₋₆-alk(en/yn)yl)amino, C₁₋₆-alk(en/yn)yl-CO—NH— and C₁₋₆-alk(en/yn)yl-sulfonamide; or two adjacent substituents may together with the Aryl group to which they are attached form a 4-8 membered ring, which is optionally substituted with one or more C₁₋₆-alk(en/yn)yl groups.

In further embodiments, the Kv7 channel activator is a compound according to formula 14, wherein: Z is O or S; q is 0; R¹ and R² are each independently selected from the group consisting of halogen, cyano, amino, C₁₋₆-alk(en/yn)yl, C₃₋₈-cycloalk(en)yl, C₃₋₈-cycloalk(en)yl-C₁₋₆-alk(en/yn)yl, Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy, and C₃₋₈-cycloalk(en)yl-C₁₋₆-alk(en/yn)yloxy; R³ is selected from the group consisting of C₁₋₈-alk(en/yn)yl, C₃₋₈-cycloalk(en)yl, C₃₋₈-cycloalk(en)yl-C₁₋₆-alk(en/yn)yl, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, amino-C₁₋₆-alk(en/yn)yl, amino-C₃₋₈-cycloalk(en)yl, amino-C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; and R⁴ is selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, Aryl, Aryl-C₃₋₈-cycloalk(en)yl, Aryl-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, NR⁵R⁶ and R⁷NH—C₁₋₆-alk(en/yn)yl; wherein: R⁵ and R⁶ are each independently selected from the group consisting of hydrogen, Aryl-C₁₋₆-alk(en/yn)yl, Aryl-C₃₋₈-cycloalk(en)yl, Aryl-C₃₋₈-cycloalk(en)yl-C₁₋₆-alk(en/yn)yl, C₁₋₆-alk(en/yn)yl, C₃₋₈-cycloalk(en)yl, and C₃₋₈-cycloalk(en)yl-C₁₋₆-alk(en/yn)yl, with the proviso that R⁵ and R⁶ cannot both be hydrogen; and R⁷ is selected from the group consisting of C₁₋₆-alk(en/yn)yl, C₃₋₈-cycloalk(en)yl, C₃₋₈-cycloalk(en)yl-C₁₋₆-alk(en/yn)yl, Aryl, halo-C₁₋₆-alk(en/yn)yl, halo-C₃₋₈-cycloalk(en)yl, halo-C₃₋₈-cycloalk(en)yl-C₁₋₆-alk(en/yn)yl, Aryl-C₁₋₆-alk(en/yn)yl, and Aryl-C₃₋₈-cycloalk(en)yl; with the proviso that the compound of formula I is not N-(4-Bromo-2,6-dimethyl-phenyl)-2-cyclopentyl-acetamide; or a pharmaceutically acceptable salt thereof; and a pharmaceutically acceptable carrier or diluent.

### Formula 15

In an embodiment, the Kv7 channel activator may be selected from one of the following compounds according to Formula 15. Such compounds are described in International Publication No. WO2009015667A1, published Feb. 5, 2009 and corresponding to International Application No. PCT/DK2008/050191 filed Jul. 31, 2008; International Publication No. WO2006092143A1, published Sep. 8, 2006 and corresponding to International Application No. PCT/DK2006/000123 filed Mar. 2, 2006; U.S. Pat. No. 7,812,020, issued Oct. 12, 2010 and corresponding to U.S. application Ser. No. 11/817,340 filed Mar. 2, 2006; US Publication No. US20100256145A1, published Oct. 7, 2010 and corresponding to U.S. application Ser. No. 12/671,505 filed Jul. 31, 2008; which are incorporated by reference in their entirety herein. In the case of any conflict of terminology in the context of Formula 15, these references incorporated by reference herein control. In an embodiment, the Kv7 channel activator is a compound according to formula 15:

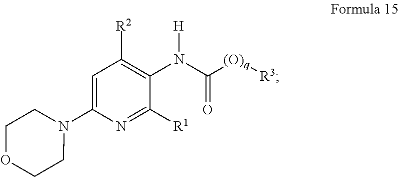

wherein, q is 0 or 1; each of R¹ and R² is independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy; and R³ is selected from the group consisting of C₁₋₈-alk(en/yn)yl, C₃₋₈-cycloalk(en)yl, C₃₋₈-cycloalk(en)yl-C₁₋₆-alk(en/yn)yl, optionally substituted Aryl-C₁₋₆-alk(en/yn)yl, optionally substituted Aryl-C₃₋₈-cycloalk(en)yl, optionally substituted Aryl-C₃₋₈-cycloalk(en)yl-C₁₋₆-alk(en/yn)yl, C₁₋₆-alk(en/yn)yl-C₃₋₈-heterocycloalk(en)yl-C₁₋₆-alk(en/yn)yl, C₃₋₈-heterocycloalk(en)yl-C₁₋₆-alk(en/yn)yl, C₁₋₆-alk(en/yn)yl, C₃₋₈-heterocycloalk(en)yl-C₁₋₆-alk(en/yn)yl, Heteroaryl-C₁₋₆-alk(en/yn)yl, Heteroaryl-C₃₋₈-cycloalk(en)yl, Heteroaryl-C₃₋₈-cycloalk(en)yl-C₁₋₆-alk(en/yn)yl, NR⁴R⁵—C₁₋₆-alk(en/yn)yl, NR⁴R⁵—C₃₋₈-cycloalk(en)yl NR⁴R⁵—C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl; wherein each of R⁴ and R⁵ is independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; as the free base or a pharmaceutically acceptable salt thereof.

In further embodiments, the Kv7 channel activator is selected from the group consisting of: (2,4-Dimethyl-6-morpholin-4-yl-pyridin-3-yl)-carbamic acid benzyl ester; (2,4-Dimethyl-6-morpholin-4-yl-pyridin-3-yl)-carbamic acid 2-chloro-benzyl ester; 2-(4-Chloro-phenyl)-(2,4-dimethyl-6-morpholin-4-yl-pyridin-3-yl)-acetamide; 2-Phenyl-cyclopropanecarboxylic acid (2,4-dimethyl-6-morpholin-4-yl-pyridin-3-yl)-amide; N-(2,4-Dimethyl-6-morpholin-4-yl-pyridin-3-yl)-2-thiophen-2-yl-acetamide; 3-Cyclohexyl-N-(2,4-dimethyl-6-morpholin-4-yl-pyridin-3-yl)-propionamide; (2,4-Dimethyl-6-morpholin-4-yl-pyridin-3-yl)-carbamic acid isobutyl ester; 3-(3-Chloro-phenyl)-N-(2,4-dimethyl-6-morpholin-4-yl-pyridin-3-yl)-propionamide; N-(2,4-Dimethyl-6-morpholin-4-yl-pyridin-3-yl)-2-(3,5-dimethyl-phenyl)-acetamide; N-(2,4-Dimethyl-6-morpholin-4-yl-pyridin-3-yl)-3-p-tolyl-propionamide; 2-(3-Chloro-phenyl)-N-(2,4-dimethyl-6-morpholin-4-yl-pyridin-3-yl)-acetamide; 2-(3,4-Dichloro-phenyl)-N-(2,4-dimethyl-6-morpholin-4-yl-pyridin-3-yl)-acetamide; N-(2,4-Dimethyl-6-morpholin-4-yl-pyridin-3-yl)-2-thiophen-3-yl-acetamide; N-(2,4-Dimethyl-6-morpholin-4-yl-pyridin-3-yl)-2-p-tolyl-acetamide; 2-(3-Bromo-phenyl)-N-(2,4-dimethyl-6-morpholin-4-yl-pyridin-3-yl)-acetamide; N-(2,4-Dimethyl-6-morpholin-4-yl-pyridin-3-yl)-2-(3-trifluoromethyl-phenyl)-acetamide; N-(2,4-Dimethyl-6-morpholin-4-yl-pyridin-3-yl)-2-phenyl-acetamide; 3,5,5-Trimethyl-hexanoic acid (2,4-dimethyl-6-morpholin-4-yl-pyridin-3-yl)-amide; Octanoic acid (2,4-dimethyl-6-morpholin-4-yl-pyridin-3-yl)-amide; N-(2,4-Dimethyl-6-morpholin-4-yl-pyridin-3-yl)-2-naphthalen-2-yl-acetamide; Heptanoic acid (2,4-dimethyl-6-morpholin-4-yl-pyridin-3-yl)-amide; N-(2,4-Dimethyl-6-morpholin-4-yl-pyridin-3-yl)-2-(3,4-dimethyl-phenyl)-acetamide; 2-Cyclohex-1-enyl-N-(2,4-dimethyl-6-morpholin-4-yl-pyridin-3-yl)-acetamide; N-(2,4-Dimethyl-6-morpholin-4-yl-pyridin-3-yl)-2-(4-methoxy-3-methyl-phenyl)-acetamide; N-(2,4-Dimethyl-6-morpholin-4-yl-pyridin-3-yl)-2-(4-methoxy-phenyl)-acetamide; N-(2,4-Dimethyl-6-morpholin-4-yl-pyridin-3-yl)-3-(4-methoxy-phenyl)-propionamide; N-(2,4-Dimethyl-6-morpholin-4-yl-pyridin-3-yl)-2-m-tolyl-acetamide; N-(2,4-Dimethyl-6-morpholin-4-yl-pyridin-3-yl)-2-(4-fluoro-phenyl)-acetamide; N-(2,4-Dimethyl-6-morpholin-4-yl-pyridin-3-yl)-3,3-dimethyl-butyramide; N-(2,4-Dimethyl-6-morpholin-4-yl-pyridin-3-yl)-2-(3-fluoro-phenyl)-acetamide; 2-Bicyclo[2.2.1]kept-2-yl-N-(2,4-dimethyl-6-morpholin-4-yl-pyridin-3-yl)-acetamide; 2-(3,4-Difluoro-phenyl)-N-(2,4-dimethyl-6-morpholin-4-yl-pyridin-3-yl)-acetamide; 4-Methyl-pentanoic acid (2,4-dimethyl-6-morpholin-4-yl-pyridin-3-yl)-amide; 2-Cyclopent-2-enyl-N-(2,4-dimethyl-6-morpholin-4-yl-pyridin-3-yl)-acetamide; 2-Cyclohexyl-N-(2,4-dimethyl-6-morpholin-4-yl-pyridin-3-yl)-acetamide; 5-Methyl-hexanoic acid (2,4-dimethyl-6-morpholin-4-yl-pyridin-3-yl)-amide; 2-Cyclopentyl-N-(2,4-dimethyl-6-morpholin-4-yl-pyridin-3-yl)-acetamide; 3-Cyclopentyl-N-(2,4-dimethyl-6-morpholin-4-yl-pyridin-3-yl)-propionamide; Hexanoic acid (2,4-dimethyl-6-morpholin-4-yl-pyridin-3-yl)-amide; N-(4-Chloro-2-methoxy-6-morpholin-4-yl-pyridin-3-yl)-2-cyclopentylacetamide; N-(2-Chloro-4-methoxy-6-morpholin-4-yl-pyridin-3-yl)-2-cyclopentylacetamide; N-(2-Chloro-4-methoxy-6-morpholin-4-yl-pyridin-3-yl)-3,3-dimethylbutyramide; N-(4-Chloro-2-methoxy-6-morpholin-4-yl-pyridin-3-yl)-3,3-dimethylbutyramide; N-(4-Chloro-2-methoxy-6-morpholin-4-yl-pyridin-3-yl)-propionamide; and pharmaceutically acceptable salts thereof.

In further embodiments, q is 0 or 1; R¹ and R² each is independently selected from the group consisting of halogen, cyano, C₁₋₆-alk(en/yn)yl, C₃₋₈-cycloalk(en)yl, C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, C₁₋₆-alk(en/yn)yloxy, C₃₋₈-cycloalk(en)yloxy and C₃₋₈-cycloalk(en)yl-C₁₋₆-alk(en/yn)yloxy; and R³ is selected from the group consisting of C₁₋₈-alk(en/yn)yl, C₃₋₈-cycloalk(en)yl, C₃₋₈-cycloalk(en)yl-C₁₋₆-alk(en/yn)yl, optionally substituted aryl-C₁₋₆-alk(en/yn)yl, optionally substituted aryl-C₃₋₈-cycloalk(en)yl, optionally substituted aryl-C₃₋₈-cycloalk(en)yl-C₁₋₆-alk(en/yn)yl, C₁₋₆-alk(en/yn)yl-C₃₋₈-heterocycloalk(en)yl-C₁₋₆-alk(en/yn)yl, C₃₋₈-heterocycloalk(en)yl-C₁₋₆-alk(en/yn)yl, C₁₋₆-alk(en/yn)yl-C₃₋₈-heterocycloalk(en)yl-C₁₋₆-alk(en/yn)yl, heteroaryl-C₁₋₆-alk(en/yn)yl, heteroaryl-C₃₋₈-cycloalk(en)yl, heteroaryl-C₃₋₈-cycloalk(en)yl-C₁₋₆-alk(en/yn)yl, NR⁴R⁵—C₁₋₆-alk(en/yn)yl, NR⁴R⁵—C₃₋₈-cycloalk(en)yl, NR⁴R⁵—C₃₋₈-cycloalk(en)yl-C₁₋₆-alk(en/yn)yl, C₁₋₆-alk(en/yn)yloxy-C₁₋₆-alk(en/yn)yl, C₃₋₈-cycloalk(en)yloxy-C₁₋₆-alk(en/yn)yl, C₃₋₈-cycloalk(en)yl-C₁₋₆-alk(en/yn)yloxy-C₁₋₆-alk(en/yn)yl, halo-C₁₋₆-alk(en/yn)yl, halo-C₃₋₈-cycloalk(en)yl and halo-C₃₋₈-cycloalk(en)yl-C₁₋₆-alk(en/yn)yl, wherein: R⁴ and R⁵ each is independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl.

### Formula 16

In an embodiment, the Kv7 channel activator may be selected from one of the following compounds according to Formula 16. Such compounds are described in International Publication No. WO2009015667A1, published Feb. 5, 2009 and corresponding to International Application No. PCT/DK2008/050191 filed Jul. 31, 2008; US Publication No. US20100256145A1, published Oct. 7, 2010 and corresponding to U.S. application Ser. No. 12/671,505 filed Jul. 31, 2008; which are incorporated by reference in their entirety herein. In the case of any conflict of terminology in the context of Formula 16, these references incorporated by reference herein control.

In an embodiment, the Kv7 channel activator is a compound according to formula 16:

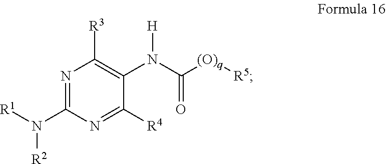

wherein: q is 0 or 1; R¹ and R² are independently selected from the group consisting of hydrogen and optionally substituted aryl-C₁₋₆-alk(en/yn)yl, provided that R¹ and R² are not both hydrogen; or R¹ and R² together with the nitrogen to which they are attached form a 5 to 7 membered ring optionally containing a further heteroatom; R² and R⁴ are independently selected from hydrogen, halogen, cyano, amino, C₁₋₆-alk(en/yn)yl, C₃₋₈-cycloalk(en)yl, halo-C₁₋₆-alk(en/yn)yl, halo-C₃₋₈-cycloalk(en)yl, C₁₋₆-alk(en/yn)yloxy, C₃₋₈-cycloalk(en)yloxy, C₃₋₈-cycloalk(en)yl-C₁₋₆-alk(en/yn)yloxy, halo-C₁₋₆-alk(en/yn)yloxy, halo-C₃₋₈-cycloalk(en)yloxy and halo-C₃₋₈-cycloalk(en)yl-C₁₋₆-alk(en/yn)yloxy, provided that R³ and R⁴ are not both hydrogen; and R⁵ is selected from the group consisting of C₁₋₁₀-alk(en/yn)yl, C₃₋₈-cycloalk(en)yl-C₁₋₆-alk(en/yn)yl, optionally substituted aryl-C₁₋₆-alk(en/yn)yl and optionally substituted aryl; as the free base or a pharmaceutically acceptable salt thereof.

In further embodiments, the Kv7 channel activator is selected from the group consisting of: N-[4-Amino-6-methyl-2-(4-trifluoromethylbenzylamino)-pyrimidin-5-yl]-2-cyclopentylacetamide; N-[4-Amino-6-methyl-2-(4-trifluoromethylbenzylamino)-pyrimidin-5-yl]-3,3-dimethylbutyramide; N-[4-Amino-6-methyl-2-(4-trifluoromethylbenzylamino)-pyrimidin-5-yl]-2-(4-fluorophenyl)-acetamide; Hexanoic acid [4-amino-6-methyl-2-(4-trifluoromethylbenzylamino)-pyrimidin-5-yl]-amide; N-[4-Amino-6-methyl-2-(4-trifluoromethylbenzylamino)-pyrimidin-5-yl]-2-(3-chlorophenyl)-acetamide; 2-Cyclopentyl-N-(4,6-dimethyl-2-morpholin-4-yl-pyrimidin-5-yl)-acetamide; N-(4,6-Dimethyl-2-morpholin-4-yl-pyrimidin-5-yl)-3,3-dimethylbutyramide; N-(4,6-Dimethyl-2-morpholin-4-ylpyrimidin-5-yl)-2-(4-fluorophenyl)-acetamide; 2-(3,4-Difluorophenyl)-N-(4,6-dimethyl-2-morpholin-4-ylpyrimidin-5-yl)-acetamide; N-(4,6-Dimethyl-2-morpholin-4-ylpyrimidin-5-yl)-2-(3-fluorophenyl)-acetamide; and Hexanoic acid (4,6-dimethyl-2-morpholin-4-ylpyrimidin-5-yl)-amide; as the free base or a pharmaceutically acceptable salt thereof.

### Formula 17

In an embodiment, the Kv7 channel activator may be selected from one of the following compounds according to Formula 17. Such compounds are described in International Publication No. WO2007065449A1, published Jun. 14, 2007 and corresponding to International Application No. PCT/DK2006/050039 filed Sep. 7, 2006; which is incorporated by reference in its entirety herein. In the case of any conflict of terminology in the context of Formula 17, this reference incorporated by reference herein controls. In an embodiment, the Kv7 channel activator is a compound according to formula 17:

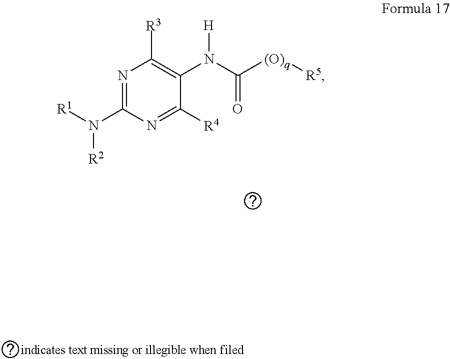

wherein: q is 0 or 1; R¹ and R² are independently selected from the group consisting of hydrogen and optionally substituted aryl-C₁₋₆-alk(en/yn)yl, provided that R¹ and R² are not both hydrogen, or R¹ and R² together with the nitrogen to which they are attached form a 5 to 7 membered ring optionally containing a further heteroatom; R³ and R⁴ are independently selected from hydrogen, halogen, cyano, amino, C₁₋₆-alk(en/yn)yl, C₃₋₈-cycloalk(en)yl, halo-C₁₋₆-alk(en/yn)yl, halo-C₃₋₈-cycloalk(en)yl, C₁₋₆-alk(en/yn)yloxy, C₃₋₈-cycloalk(en)yloxy, C₃₋₈-cycloalk(en)yl-C₁₋₆-alk(en/yn)yloxy, halo-C₁₋₆-alk(en/yn)yloxy, halo-C₃₋₈-cycloalk(en)yloxy and halo-C₃₋₈-cycloalk(en)yl-C₁₋₆-alk(en/yn)yloxy, provided that R³ and R⁴ are not both hydrogen; R⁵ is selected from the group consisting of C₁₋₁₀-alk(en/yn)yl, C₃₋₈-cycloalk(en)yl-C₁₋₆-alk(en/yn)yl, optionally substituted aryl-C₁₋₆-alk(en/yn)yl and optionally substituted aryl; as the free base or salts thereof.

In further embodiments, q is 0.

In further embodiments, q is 1.

In further embodiments, R¹ and R² are independently selected from hydrogen and optionally substituted aryl-C₁₋₆-alk(en/yn)yl, provided that R¹ and R² are not both hydrogen.

In further embodiments, R¹ and R² together with the nitrogen to which they are attached form a 5 to 7 membered ring optionally containing a further hetero atom.

In further embodiments, said further hetero atom is oxygen.

In further embodiments, said ring is a 6 membered ring.

In further embodiments, said ring is a morpholine ring.

In further embodiments, R³ and R⁴ are independently selected from amino and C₁₋₆-alk(en/yn)yl, preferably methyl.

In further embodiments, R⁵ is selected from the group consisting of C₁₋₁₀-alk(en/yn)yl, C₃₋₈-cycloalk(en)yl-C₁₋₆-alk(en/yn)yl, optionally substituted aryl-C₁₋₆-alk(en/yn)yl and optionally substituted aryl.

In further embodiments, the Kv7 Channel activator is selected from the group consisting of: N-[4-Amino-6-methyl-2-(4-trifluoromethylbenzylamino)-pyrimidin-5-yl]-2-cyclopentylacetamide, N-[4-Amino-6-methyl-2-(4-trifluoromethylbenzylamino)-pyrimidin-5-yl]-3,3-dimethylbutyramide, N-[4-Amino-6-methyl-2-(4-trifluoromethylbenzylamino)-pyrimidin-5-yl]-2-(4-fluorophenyl)-acetamide, Hexanoic acid [4-amino-6-methyl-2-(4-trifluoromethylbenzylamino)-pyrimidin-5-yl]-amide, N-[4-Amino-6-methyl-2-(4-trifluoromethylbenzylamino)-pyrimidin-5-yl]-2-(3-chlorophenyl)-acetamide, 2-Cyclopentyl-N-(4,6-dimethyl-2-moφholin-4-yl-pyrimidin-5-yl)-acetamide, N-(4,6-Dimethyl-2-moφholin-4-yl-pyrimidin-5-yl)-3,3-dimethylbutyramide, N-(4,6-Dimethyl-2-mopholin-4-ylpyrimidin-5-yl)-2-(4-fluorophenyl)-acetamide, 2-(3,4-Difluorophenyl)-N-(4,6-dimethyl-2-morpholin-4-ylpyrimidin-5-yl)-acetamide, N-(4,6-Dimethyl-2-moφholin-4-ylpyrimidin-5-yl)-2-(3-fluorophenyl)-acetamide and Hexanoic acid (4,6-dimethyl-2-mopholin-4-ylpyrimidin-5-yl)-amide; as the free base or a salt thereof.

### Formula 18

In an embodiment, the Kv7 channel activator may be selected from one of the following compounds according to Formula 18. Such compounds are described in International Publication No. WO2004096767A1, published Nov. 11, 2004 and corresponding to International Application No. PCT/DK2004/000283 filed Apr. 23, 2004; US Publication No. US20060264496A1, published Nov. 23, 2006 and corresponding to U.S. application Ser. No. 10/551,783 filed Apr. 23, 2004; which are incorporated by reference in their entirety herein. In the case of any conflict of terminology in the context of Formula 18, these references incorporated by reference herein control.

In an embodiment, the Kv7 channel activator is a compound according to formula 18:

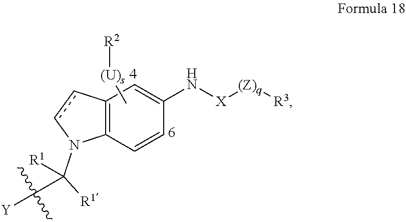

wherein, the dotted line represents an optional bond; R¹ and R^{1′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃-8-cycloalk(en)yl and cyano-C₃₋₈-cycloalk(en)yl-C₁₋₆-alk(en/yn)yl; or R¹ and R^{1′} together with the carbon atom to which they are attached form a 3-8 membered saturated or unsaturated ring which optionally contains 1 or 2 heteroatoms; s is 0 or 1; U is O, NR¹¹, S, SO₂, SO₂NR¹¹, CO—O or CO—NR¹¹; wherein R¹¹ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl; or R² and R¹¹ together with the nitrogen atom to which they are attached form a 4-8 membered saturated or unsaturated ring which optionally contains 1, 2 or 3 further heteroatoms; R² is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, acyl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NO₂, NR¹⁰R^{10′}-C₁₋₆-alk(en/yn)yl, NR¹⁰R^{10′}-C₃₋₈-cycloalk(en)yl and NR¹⁰R^{10′}-C₃₋₈-cycloalk(en)yl-C₁₋₆-alk(en/yn)yl; wherein R¹⁰ and R^{10′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or R¹⁰ and R^{10′} together with the nitrogen atom to which they are attached form a 4-8 membered saturated or unsaturated ring which optionally contains 1, 2 or 3 further heteroatoms; with the proviso that when R² is NO₂, halogen or cyano then s is 0; and with the proviso that when R² is a hydrogen atom or acyl and s is 1 then U is NR¹¹, O or S; wherein the group -(U)ₛ-R² is linked to position 4 or 6 of the indole or indoline; q is 0 or 1; Z is O or S; X is CO or SO₂; with the proviso that q is 0 when X is SO₂; R³ is selected from the group consisting of C₁₋₆-alk(en/yn)yl, C₃₋₈-cycloalk(en)yl, heterocycloalk(en)yl, C₃₋₈-cycloalk(en)yl-C₁₋₆-alk(en/yn)yl, C₁₋₆-alk(en/yn)yl-C₃₋₈-cycloalk(en)yl, C₁₋₆-alk(en/yn)yl-heterocycloalk(en)yl, Ar, Ar-C₁₋₆-alk(en/yn)yl, Ar-C₃₋₈-cycloalk(en)yl, Ar-heterocycloalk(en)yl, Ar-C₃₋₈-cycloalk(en)yl-C₁₋₆-alk(en/yn)yl, Ar-C₁₋₆-alk(en/yn)yl-C₃₋₈-cycloalk(en)yl, Ar-C₁₋₆-alk(en/yn)yl-heterocycloalk(en)yl, C₁₋₆-alk(en/yn)yloxy-C₁₋₆-alk(en/yn)yl, C₃₋₈-cycloalk(en)yloxy-C₁₋₆-alk(en/yn)yl, C₁₋₆-alk(en/yn)yloxy-C₃₋₈-cycloalk(en)yl, C₁₋₆-alk(en/yn)yloxy-heterocycloalk(en)yl, Ar-oxy-C₁₋₆-alk(en/yn)yl, Ar-C₁₋₆-alk(en/yn)yloxy-C₁₋₆-alk(en/yn)yl, C₁₋₆-alk(en/yn)yloxy-carbonyl-C₁₋₆-alk(en/yn)yl, C₃₋₈-cycloalk(en)yloxy-carbonyl-C₁₋₆-alk(en/yn)yl, C₃₋₈-cycloalk(en)yl-C₁₋₆-alk(en/yn)yloxy-carbonyl-C₁₋₆-alk(en/yn)yl, hydroxy-C₁₋₆-alk(en/yn)yl, hydroxy-C₃₋₈-cycloalk(en)yl, hydroxy-heterocycloalk(en)yl, hydroxy-C₃₋₈-cycloalk(en)yl-C₁₋₆-alk(en/yn)yl, hydroxy-C₁₋₆-alk(en/yn)yl-C₃₋₈-cycloalk(en)yl, hydroxy-C₁₋₆-alk(en/yn)yl-heterocycloalk(en)yl, halo-C₁₋₆-alk(en/yn)yl, halo-C₃₋₈-cycloalk(en)yl, halo-heterocycloalk(en)yl, halo-C₃₋₈-cycloalk(en)yl-C₁₋₆-alk(en/yn)yl, halo-C₁₋₆-alk(en/yn)yl-C₃₋₈-cycloalk(en)yl, halo-C₁₋₆-alk(en/yn)yl-heterocycloalk(en)yl, halo-C₁₋₆-alk(en/yn)yl-Ar, halo-C₃₋₈-cycloalk(en)yl-Ar, halo-C₃₋₈-cycloalk(en)yl-C₁₋₆-alk(en/yn)yl-Ar, halo-C₁₋₆-alk(en/yn)yl-C₃₋₈-cycloalk(en)yl-Ar, cyano-C₁₋₆-alk(en/yn)yl, cyano-C₃₋₈-cycloalk(en)yl, cyano-heterocycloalk(en)yl, cyano-C₃₋₈-cycloalk(en)yl-C₁₋₆-alk(en/yn)yl, cyano-C₁₋₆-alk(en/yn)yl-C₃₋₈-cycloalk(en)yl, cyano-C₁₋₆-alk(en/yn)yl-heterocycloalk(en)yl, acyl-C₁₋₆-alk(en/yn)yl, acyl-C₃₋₈-cycloalk(en)yl, acyl-heterocycloalk(en)yl, acyl-C₃₋₈-cycloalk(en)yl-C₁₋₆-alk(en/yn)yl, acyl-C₁₋₆-alk(en/yn)yl-C₃₋₈-cycloalk(en)yl, acyl-C₁₋₆-alk(en/yn)yl-heterocycloalk(en)yl and —NR¹²R^{12′}, optionally substituted NR¹²R^{12′}-C₁₋₆-alk(en/yn)yl, optionally substituted NR¹²R^{12′}-C₃₋₈-cycloalk(en)yl, optionally substituted NR¹²R^{12′}-C₃₋₈-cycloalk(en)yl-C₁₋₆-alk(en/yn)yl; wherein R¹² and R^{12′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-C₃₋₈-cycloalk(en)yl, Ar-C₃₋₈-cycloalk(en)yl-C₁₋₆-alk(en/yn)yl, hydroxy-C₁₋₆-alk(en/yn)yl, hydroxy-C₃₋₈-cycloalk(en)yl, hydroxy-C₃₋₈-cycloalk(en)yl-C₁₋₆-alk(en/yn)yl, halo-C₁₋₆-alk(en/yn)yl, halo-C₃₋₈-cycloalk(en)yl, halo-C₃₋₈-cycloalk(en)yl-C₁₋₆-alk(en/yn)yl, cyano-C₁₋₆-alk(en/yn)yl, cyano-C₃₋₈-cycloalk(en)yl and cyano-C₃₋₈-cycloalk(en)yl-C₁₋₆-alk(en/yn)yl, or R¹² and R^{12′} together with the nitrogen atom to which they are attached form a 4-8 membered saturated or unsaturated ring which optionally contains 1, 2 or 3 further heteroatoms; with the proviso that when R³ is NR¹²R^{12′} then q is 0; and Y is selected from the groups according to the following formulas:

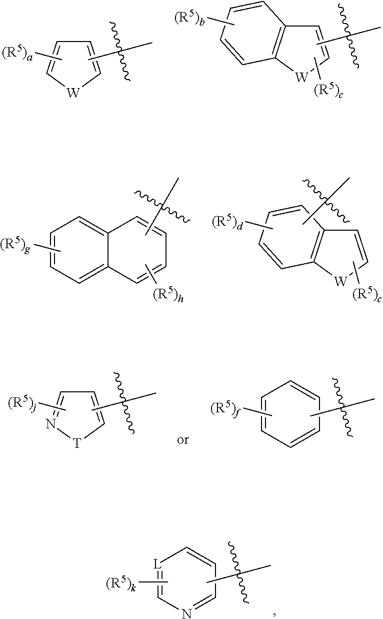

wherein, W is O or S; T is N, NH or O; L is N, C or CH; a is 0, 1, 2 or 3; b is 0, 1, 2, 3 or 4; c is 0 or 1; d is 0, 1, 2 or 3; e is 0, 1 or 2; f is 0, 1, 2, 3, 4 or 5; g is 0, 1, 2, 3 or 4; h is 0, 1, 2 or 3; j is 0, 1, 2 or 3; with the proviso that when T is a nitrogen atom then j is 0, 1, 2 or 3; and when T is NH or an oxygen atom then j is 0, 1 or 2; k is 0, 1, 2, 3 or 4; and each R⁵ is independently selected from the group consisting of a C₁₋₆-alk(en/yn)yl, C₃₋₈-cycloalk(en)yl, C₃₋₈-cycloalk(en)yl-C₁₋₆-alk(en/yn)yl, Ar, Ar-C₁₋₆-alk(en/yn)yl, Ar-thio, Ar-oxy, acyl, C₁₋₆-alk(en/yn)yloxy, C₃₋₈-cycloalk(en)yloxy, C₃₋₈-cycloalk(en)yl-C₁₋₆-alk(en/yn)yloxy, halogen, halo-C₁₋₆-alk(en/yn)yl, halo-C₃₋₈-cycloalk(en)yl, halo-C₃₋₈-cycloalk(en)yl-C₁₋₆-alk(en/yn)yl, —CO—NR⁶R^{6′}, cyano, cyano-C₁₋₆-alk(en/yn)yl, cyano-C₃₋₈-cycloalk(en)yl, cyano-C₃₋₈-cycloalk(en)yl-C₁₋₆-alk(en/yn)yl, —NR⁷R^{7′}, —S—R⁸ and —SO₂R⁸, or two adjacent R⁵ together with the aromatic group to which they are attached form a 4-8 membered ring which optionally contains one or two heteroatoms; R⁶ and R^{6′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl and Ar; R⁷ and R^{7′} are independently selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and acyl; and R⁸ is selected from the group consisting of hydrogen, C₁₋₆-alk(en/yn)yl, C₃₋₈-cycloalk(en)yl, C₃₋₈-cycloalk(en)yl-C₁₋₆-alk(en/yn)yl, Ar and —NR⁹R^{9′}; wherein R⁹ and R^{9′} are independently selected from the group consisting of hydrogen, C₁₋₆-alk(en/yn)yl, C₃₋₈-cycloalk(en)yl and C₃₋₈-cycloalk(en)yl-C₁₋₆-alk(en/yn)yl; provided that when R⁸ is —NR⁹R^{9′} then R⁵ is not —S—R⁸; or salts thereof.

### Formula 19

In an embodiment, the Kv7 channel activator may be selected from one of the following compounds according to Formula 19. Such compounds are described in U.S. Pat. No. 9,248,122, issued Feb. 2, 2016 and corresponding to U.S. application Ser. No. 14/091,395 filed Nov. 27, 2013; which is incorporated by reference in its entirety herein. In the case of any conflict of terminology in the context of Formula 19, this reference incorporated by reference herein controls.

In an embodiment, the Kv7 channel activator is a compound according to formula 19:

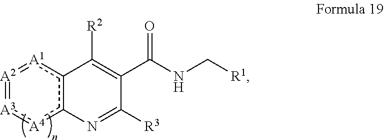

wherein, A¹, A² and A³ independently of each other represent CR⁴, N, O, S or N(CH₃), A⁴ represents CR⁴ or N, and n denotes 0 or 1, with the proviso, that at least one of A¹, A², A³ and A⁴ does not represent CR⁴, and with the proviso, that if n denotes 0, then precisely one of A¹, A² and A³ represents O, S or N(CH₃), or if n denotes 1, then A¹, A² and A³ independently of each other represent CR⁴ or N, R¹ represents C₁₋₁₀-aliphatic residue, unsubstituted or mono- or polysubstituted; C₃₋₁₀-cycloaliphatic residue or 3 to 10 membered heterocycloaliphatic residue, in each case unsubstituted or mono- or polysubstituted and in each case optionally linked via a C₁₋₈-aliphatic group, which in turn may be unsubstituted or mono- or polysubstituted; aryl or heteroaryl, in each case unsubstituted or mono- or polysubstituted and in each case optionally linked via a C₁₋₈-aliphatic group, which in turn may be unsubstituted or mono- or polysubstituted; R² represents F; Cl; Br; I; CN; CF₃; C(═O) H; NO₂; OCF₃; SCF₃; C₁₋₄-aliphatic residue, C(═O)—C₁₋₄-aliphatic residue, C(═O)—O—C₁₋₄-aliphatic residue, C(═O)—NH—C₁₋₄-aliphatic residue, C(═O)—N(C₁₋₄-aliphatic residue)₂, O—C₁₋₄-aliphatic residue, O—C(═O)—C₁₋₄-aliphatic residue, S—C₁₋₄-aliphatic residue, S(═O)₂-C₁₋₄-aliphatic residue, S(═O)₂—O—C₁₋₄-aliphatic residue, wherein the C₁₋₄-aliphatic residue may be in each case be unsubstituted or mono- or polysubstituted; C₃₋₆-cycloaliphatic residue or 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted; R³ represents C₂₋₁₀-aliphatic residue, unsubstituted or mono- or polysubstituted; C₃₋₁₀-cycloaliphatic residue or 3 to 10 membered heterocycloaliphatic residue, in each case unsubstituted or mono- or polysubstituted and in each case optionally linked via a C₁₋₈-aliphatic group, which in turn may be unsubstituted or mono- or polysubstituted, or denotes S—R⁵, O—R⁶ or N(R⁷R⁸), wherein R⁵ and R⁶ in each case represent C₁₋₁₀-aliphatic residue, unsubstituted or mono- or polysubstituted; C₃₋₁₀-cycloaliphatic residue or 3 to 10 membered heterocycloaliphatic residue, in each case unsubstituted or mono- or polysubstituted and in each case optionally linked via a C₁₋₈-aliphatic group, which in turn may be unsubstituted or mono- or polysubstituted; with the proviso, that if R⁵ or R⁶ denote a 3 to 10 membered heterocycloaliphatic residue, than the 3 to 10 membered heterocycloaliphatic residue is linked via a carbon atom, R⁷ represents C₁₋₁₀-aliphatic residue, unsubstituted or mono- or polysubstituted; C₃₋₁₀-cycloaliphatic residue or 3 to 10 membered heterocycloaliphatic residue, in each case unsubstituted or mono- or polysubstituted and in each case optionally linked via a C₁₋₈-aliphatic group, which in turn may be unsubstituted or mono- or polysubstituted; with the proviso that if R⁷ denotes 3 to 10 membered heterocycloaliphatic residue, the 3 to 10 membered heterocycloaliphatic residue is linked via a carbon atom; and R⁸ denotes C₁₋₁₀-aliphatic residue, unsubstituted or mono- or polysubstituted; or R⁷ and R⁸ form together with the nitrogen atom connecting them a 3 to 10 membered heterocycloaliphatic residue, unsubstituted or mono- or polysubstituted; and each R⁴ independently represents H, F; Cl; Br; I; CN; CF₃; CHF₂; CH₂F; OCF₃; OCHF₂; OCH₂F; SCF₃; O—C₁₋₄-aliphatic residue, C₁₋₄-aliphatic residue or S(═O)₂-C₁₋₄-aliphatic residue; in which an “aliphatic group” and “aliphatic residue” may in each case be branched or unbranched, saturated or unsaturated, in which a “cycloaliphatic residue” and a “heterocycloaliphatic residue” may in each case be saturated or unsaturated, in which “mono- or polysubstituted” with respect to an “aliphatic group”, an “aliphatic residue”, a “cycloaliphatic residue” and a “heterocycloaliphatic residue” relates, with respect to the corresponding residues or groups, to the substitution of one or more hydrogen atoms each independently of one another by at least one substituent selected from the group consisting of F, Cl, Br, I, NO₂, NH₂, NH(C₁₋₄-aliphatic residue), N(C₁₋₄-aliphatic residue)₂, NH—C(═O)—C₁₋₄aliphatic residue, N(C₁₋₄aliphatic residue)-C(═O)—C₁₋₄ aliphatic residue, NH—S(═O)₂-C₁₋₄ aliphatic residue, N(C₁₋₄ aliphatic residue)-S(═O)₂-C₁₋₄ aliphatic residue, ═O, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂-C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, CHO, COOH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, C(═O)NH₂, a C(═O)—NH(C₁₋₄-aliphatic residue) and C(═O)—N(C₁₋₄-aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of

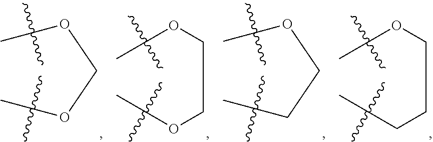 [SMILES: *OCO* ; *OCCO* ; *CCO* ; *CCCO*]

F, Cl, Br, I, NO₂, NH₂ NH(C₁₋₄-aliphatic residue), N(C₁₋₄-aliphatic residue)₂, NH—C(O)—C₁₋₄-aliphatic residue, N(C₁₋₄ aliphatic residue)-C(═O)—C₁₋₄ aliphatic residue, NH—S(═O)₂-C₁₋₄ aliphatic residue, N(C₁₋₄ aliphatic residue)-S(═O)₂-C₁₋₄ aliphatic residue, OH, OCF₃, O—C₁₋₄-aliphatic residue, O—C(═O)-C₁₋₄-aliphatic residue, SH, SCF₃, S—C₁₋₄-aliphatic residue, S(═O)₂OH, S(═O)₂-C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, C(═O) H, C(═O) OH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, C(═O)—NH(C₁₋₄-aliphatic residue) and C(═O)—N(C₁₋₄-aliphatic residue)₂; in the form of an individual single stereoisomer or a mixture of the stereoisomers in any mixing ratio, and/or in the form of a free compound, a solvate and/or a physiologically acceptable salt.

In further embodiments, A¹, A² and A³ independently of each other represent CR⁴, N, O, S or N(CH₃), A⁴ represents CR⁴ or N, and n denotes 0 or 1, with the proviso, that at least one of A¹, A², A³ and A⁴ does not represent CR⁴, and with the proviso, that if n denotes 0, then precisely one of A¹, A² and A³ represents O, S or N(CH₃), or if n denotes 1, then A¹, A² and A³ independently of each other represent CR⁴ or N, R¹ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₈-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₈-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O)OH, and wherein the C₃₋₁₀-cycloaliphatic residue or the 3 to 10 membered heterocycloaliphatic residue may in each case optionally linked via a C₁₋₈-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, O₃₋₈-cycloaliphatic residue, 3 to 7 membered heterocycloaliphatic residue,

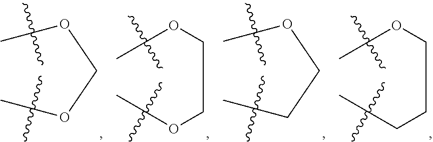 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the aryl or the heteroaryl residue may in each case be optionally linked via a C₁₋₈-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O) OH, R² represents F; Cl; Br; I; CN; CF₃; NO₂; OCF₃; SCF₃; C₁₋₄-aliphatic residue, S—C₁₋₄-aliphatic residue, O—C₁₋₄-aliphatic residue, wherein the C₁₋₄-aliphatic residue may be in each case be unsubstituted or mono- or polysubstituted with at least one substituent selected from the group consisting of F, Cl, Br, I, OH, OCF₃, CF₃ and O—C₁₋₄-aliphatic residue,
Or
C₃₋₆-cycloaliphatic residue or 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O) OH, R³ denotes C₂₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue,
Or
denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₁₀-cycloaliphatic residue or the 3 to 10 membered heterocycloaliphatic residue may in each case optionally linked via a C₁₋₈-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH,
Or
denotes S—R⁵, O—R⁶ or N(R⁷R⁸), wherein R⁵ and R⁶ in each case represent C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, or in each case represent C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₈-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₈-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the C₃₋₁₀-cycloaliphatic residue or the 3 to 10 membered heterocycloaliphatic residue may in each case optionally linked via a C₁₋₈-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, on the condition that if R⁵ or R⁶ denote a 3 to 10 membered heterocycloaliphatic residue, the 3 to 10 membered heterocycloaliphatic residue is linked via a carbon atom, R⁷ denotes a C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue, C(═O) OH, C(═O)—O—C₁₋₄-aliphatic residue, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the C₃₋₁₀-cycloaliphatic residue or the 3 to 10 membered heterocycloaliphatic residue may in each case optionally linked via a O₁₋₀-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O) OH, on the condition that if R⁷ denotes a 3 to 10 membered heterocycloaliphatic residue, the 3 to 10 membered heterocycloaliphatic residue is linked via a carbon atom,
and/or
R⁸ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue,
Or
R⁷ and R⁸ form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue,
And/or
wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the 3 to 10 membered heterocycloaliphatic residue formed by R⁷ and R⁸ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, C₃₋₆-cycloaliphatic residue, 3 to 7 membered heterocycloaliphatic residue,

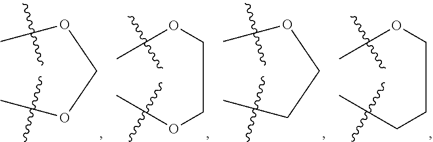 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, and wherein the C₃₋₆ cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH, and each R⁴ independently represents H, F, Cl, Br, I, CN, CF₃, CHF₂, CH₂F, OCF₃, OCHF₂, OCH₂F, SCF₃, O—C₁₋₄-aliphatic residue, C₁₋₄-aliphatic residue or S(═O)₂-C₁₋₄-aliphatic residue. In further embodiments, n denotes 1 and A¹ represents N, A² represents CR⁴, A³ represents CR⁴ and A⁴ represents CR⁴; or n denotes 1 and A¹ represents CR⁴, A² represents N, A³ represents CR⁴ and A⁴ represents CR⁴; or n denotes 1 and A¹ represents CR⁴, A² represents CR⁴, A³ represents N and A⁴ represents CR⁴; or n denotes 1 and A¹ represents CR⁴, A² represents CR⁴, A³ represents CR⁴ and A⁴ represents N; or n denotes 1 and A¹ represents N, A² represents N, A³ represents CR⁴ and A⁴ represents CR⁴; or n denotes 1 and A¹ represents N, A² represents CR⁴, A³ represents N and A⁴ represents CR⁴; or n denotes 1 and A¹ represents N, A² represents CR⁴, A³ represents CR⁴ and A⁴ represents N; or n denotes 1 and A¹ represents CR⁴, A² represents N, A³ represents N and A⁴ represents CR⁴; or n denotes 1 and A¹ represents CR⁴, A² represents N, A³ represents CR⁴ and A⁴ represents N; or n denotes 1 and A¹ represents CR⁴, A² represents CR⁴, A³ represents N and A⁴ represents N; or n denotes 0 and A¹ represents CR⁴, A² represents CR⁴, and A³ represents S; or n denotes 0 and A¹ represents N, A² represents CR⁴, and A³ represents S; or n denotes 0 and A¹ represents S, A² represents CR⁴ and A³ represents CR⁴; or n denotes 0 and A¹ represents S, A² represents CR⁴ and A³ represents N.

In further embodiments, R² represents F; Cl; Br; I; CN; CF₃; NO₂; OCF₃; SCF₃; C₁₋₄-aliphatic residue, S—C₁₋₄-aliphatic residue or O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH and O—C₁₋₄-aliphatic residue.

In further embodiments, R² represents C₁₋₄-aliphatic residue.

In further embodiments, each R⁴ independently represents H; F; Cl; Br; CN; CF₃; OCF₃; CH₃, OCH₃ or S(═O)₂CH₃.

In further embodiments, R¹ represents the partial structure:

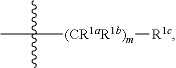

wherein, m denotes 0, 1, or 2, R^{1a} and R^{1b} each independently of one another represent H, F, Cl, Br, I, O—C₁₋₄-aliphatic residue or C₁₋₄-aliphatic residue, R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and O—C₁₋₄-aliphatic residue, or denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and wherein the C₃₋₆ cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃ C₁₋₄-aliphatic residue and C(═O) OH.

In further embodiments, m denotes 1 or 2, R^{1a} and R^{1b} represent H, R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue, or m denotes 0 and R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃, C₁₋₄-aliphatic residue and C(═O) OH.

In further embodiments, R³ denotes a C₂₋₆-aliphatic residue, unsubstituted or mono- or polysubstituted with at least one substituent selected from the group consisting of F, Cl, Br, I, OH, ═O, O—C₁₋₄-aliphatic residue, OCF₃, SH, SCF₃, S—C₁₋₄-aliphatic residue, CF₃, CN and C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and O—C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, OH, ═O, O—C₁₋₄-aliphatic residue, OCF₃, SCF₃, (═O)—O—C₁₋₄-aliphatic residue, S—C₁₋₄-aliphatic residue, CF₃, CN and C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₇-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, OH, ═O, O—C₁₋₄-aliphatic residue, OCF₃, SCF₃, CF₃, CN and C₁₋₄-aliphatic residue, or R³ denotes S—R⁵ or O—R⁶, wherein R⁵ and R⁶ in each case denote C₁₋₈-aliphatic residue, unsubstituted or mono- or polysubstituted with at least one substituent selected from the group consisting of F, Cl, Br, I, OH, —O, O—C₁₋₄-aliphatic residue, OCF₃, SH, SCF₃, S—C₁₋₄-aliphatic residue, NH(C₁₋₄-aliphatic residue), N(C₁₋₄-aliphatic residue)₂, CF₃ and C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and O—C₁₋₄-aliphatic residue, or in each case denote C₃₋₆-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, C₁, OH, OCF₃, CF₃ and O—C₁₋₄-aliphatic residue, and wherein the C₃₋₆-cycloaliphatic residue or the 3 to 7 membered heterocycloaliphatic residue may be linked via a C₁₋₄-aliphatic group, which in turn may be unsubstituted or mono- or polysubstituted with at least one substituent selected from the group consisting of F, Cl, OH, —O, O—C₁₋₄-aliphatic residue, OCF₃, CF₃, CN and C₁₋₄-aliphatic residue, on the condition that if R⁵ or R⁶ denotes 3 to 7 membered heterocycloaliphatic residue, the 3 to 7 membered heterocycloaliphatic residue is linked via a carbon atom, or R³ denotes N(R⁷R⁸), wherein R⁷ denotes C₁₋₆-aliphatic residue, unsubstituted or mono- or polysubstituted with at least one substituent selected from the group consisting of F, Cl, Br, I, OH, ═O, O—C₁₋₄-aliphatic residue, OCF₃, SCF₃, CF₃ and C₁₋₄-aliphatic residue wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, Cl, OH, OCF₃, CF₃ and O—C₁₋₄-aliphatic residue, and wherein the C₃₋₆-cycloaliphatic residue or the 3 to 7 membered heterocycloaliphatic residue may in each case be linked, via a C₁₋₄-aliphatic group, which in turn may be unsubstituted or mono- or polysubstituted with at least one substituent selected from the group consisting of F, Cl, OH, O, Q-C₁₋₄-aliphatic residue, OCF₃, SCF₃, CF₃, CN and C₁₋₄-aliphatic residue, on the condition that if R⁷ denotes 3 to 7 membered heterocycloaliphatic residue, the 3 to 7 membered heterocycloaliphatic residue is linked via a carbon atom, and R⁸ denotes C₁₋₆-aliphatic residue, unsubstituted or mono- or polysubstituted with at least one substituent selected from the group consisting of F, Cl, OH, —O, O—C₁₋₄-aliphatic residue, OCF₃, SCF₃, CF₃, CN and C₁₋₄-aliphatic residue, wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, or R⁷ and R⁸ form together with the nitrogen atom connecting them a 3 to 10 membered heterocycloaliphatic residue, unsubstituted or mono- or polysubstituted with at least one substituent selected from the group consisting of F, Cl, OH, ═O, O—C₁₋₄-aliphatic residue, OCF₃, SCF₃, CF₃, CN and C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and O—C₁₋₄-aliphatic residue, and wherein the 3 to 10 membered heterocycloaliphatic residue formed by R⁷ and R⁸ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, OH, O—C₁₋₄-aliphatic residue, OCF₃, SCF₃, CF₃, ON, C₁₋₄-aliphatic residue, C(═O) OH, C₃₋₆ cycloaliphatic residue, 3 to 7 membered heterocycloaliphatic residue,

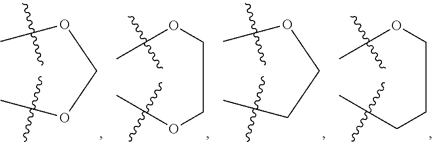 [SMILES: *OCO* ; *OCCO* ; *CCO* ; *CCCO*]

benzyl, phenyl, thienyl, and pyridyl, wherein the C₁₋₄-aliphatic residue in each case may be unsubstituted or mono- or polysubstituted with at least one substituent selected from the group consisting of F, Cl, OH, OCF₃, CF₃ and O—C₁₋₄-aliphatic residue, and wherein benzyl, phenyl, thienyl, and pyridyl, may in each case may be unsubstituted or mono- or polysubstituted with at least one substituent selected from the group consisting of F, Cl, OH, O—C₁₋₄-aliphatic residue, OCF₃, OCH₂CH₂OH, OCH₂OCH₃, SCF₃, CF₃, CN, C₁₋₄-aliphatic residue and C(═O) OH, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, OH, ═O, O—C₁₋₄-aliphatic residue, OCF₃, CF₃, CN, C₁₋₄-aliphatic residue and C(═O) OH.

In further embodiments, R³ denotes C₂₋₆-aliphatic residue, unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, OH, —O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with OH or O—C₁₋₄-aliphatic residue, and wherein the C₃₋₆-cycloaliphatic residue or the 3 to 7 membered heterocycloaliphatic residue may in each case optionally linked via a C₁₋₄-aliphatic group, or R³ denotes S—R⁵ or O—R⁶, wherein R⁵ and R⁶ in each case denote C₁₋₆-aliphatic residue, unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, or in each case denote C₃₋₆-cycloaliphatic residue or 3 to 7 membered heterocycloaliphatic residue, in each case unsubstituted or mono- or polysubstituted with at least one substituent selected from the group consisting of F, Cl, OH, —O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, Cl, OH, OCF₃, CF₃ and O—C₁₋₄-aliphatic residue, and wherein the C₃₋₆-cycloaliphatic residue or the 3 to 7 membered heterocycloaliphatic residue in each case may be linked via an unsubstituted C₁₋₄-aliphatic group, on the condition that if R⁵ or R⁶ denotes 3 to 7 membered heterocycloaliphatic residue, the 3 to 7 membered heterocycloaliphatic residue is linked via a carbon atom, or R³ denotes N(R⁷R⁸), wherein R⁷ denotes C₁₋₆-aliphatic residue, unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, OH, —O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, C₁, OH, OCF₃, CF₃ and O—C₁₋₄-aliphatic residue, and wherein the C₃₋₆-cycloaliphatic residue or the 3 to 7 membered heterocycloaliphatic residue is in each case linked via a unsubstituted C₁₋₄-aliphatic group, on the condition that if R⁷ denotes 3 to 7 membered heterocycloaliphatic residue, the 3 to 7 membered heterocycloaliphatic residue is linked via a carbon atom, and R⁸ denotes unsubstituted C₁₋₄-aliphatic residue, or R⁷ and R⁸ form together with the nitrogen atom connecting them a 3 to 7 membered heterocycloaliphatic residue, unsubstituted or mono- or polysubstituted with at least one substituent selected from the group consisting of F, C₁, OH, ═O, O—C₁₋₄-aliphatic residue, OCF₃, SCF₃, CF₃, CN and C₁₋₄-aliphatic residue, wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, and wherein the 3 to 7 membered heterocycloaliphatic residue formed by R⁷ and R⁸ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, OH, O—C₁₋₄-aliphatic residue, OCF₃, SCF₃, CF₃, CN, C₁₋₄-aliphatic residue, C(═O) OH, benzyl, phenyl, and pyridyl, wherein the C₁₋₄-aliphatic residue in each case may be unsubstituted or mono- or polysubstituted with at least one substituent selected from the group consisting of F, Cl, OH and O—C₁₋₄-aliphatic residue, and wherein benzyl, phenyl, and pyridyl, may in each case may be unsubstituted or mono- or polysubstituted with at least one substituent selected from the group consisting of F, Cl, OCH₃, OCF₃, OCH₂CH₂OH, OCH₂CH₂OCH₃, SCF₃, CF₃ and C₁₋₄-aliphatic residue.

In further embodiments, A¹, A² and A³ independently of each other represent CR⁴, N, O, S or N(CH₃), A⁴ represents CR⁴ or N, and n denotes 0 or 1, with the proviso, that at least one of A¹, A², A³ and A⁴ does not represent CR⁴, and with the proviso, that if n denotes 0, then precisely one of A¹, A² and A³ represents O, S or N(CH₃), or if n denotes 1, then A¹, A² and A³ independently of each other represent CR⁴ or N, R¹ represents the partial structure:

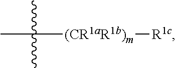

wherein: m denotes 0, 1, or 2, R^{1a} and R^{1b} each independently of one another represent H, F, C₁, O—C₁₋₄-aliphatic residue or C₁₋₄-aliphatic residue, R^{1c} denotes C₁₋₄-aliphatic residue, unsubstituted or mono- or polysubstituted with at least one substituent selected from the group consisting of F, C₁₋₁₀-C₁₋₄ aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, C₁₋₁₀-C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and O—C₁₋₄-aliphatic residue, or denotes aryl or heteroaryl, in each case unsubstituted or mono- or polysubstituted with at least one substituent selected from the group consisting of F, Cl, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃ C₁₋₄-aliphatic residue and C(═O) OH, R² represents F; Cl; Br; I; CN; CF₃; NO₂; OCF₃; SCF₃; C₁₋₄-aliphatic residue, S—C₁₋₄-aliphatic residue or O—C₁₋₄-aliphatic residue, wherein the C₁₋₄-aliphatic residue in each case may be unsubstituted or mono- or polysubstituted with at least one substituent selected from the group consisting of F, Cl, ═O, OH and O—C₁₋₄-aliphatic residue, R³ denotes C₂₋₆-aliphatic residue, unsubstituted or mono- or polysubstituted with at least one substituent selected from the group consisting of F, Cl, OH, —O, O—C₁₋₄-aliphatic residue, OCF₃, SCF₃, CF₃ and C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, OH, —O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with OH or O—C₁₋₄-aliphatic residue, and wherein the C₃₋₁₀-cycloaliphatic residue or the 3 to 10 membered heterocycloaliphatic residue may in each case optionally linked via a C₁₋₄-aliphatic group, or R³ denotes S—R⁵ or O—R⁶, wherein R⁵ and R⁶ in each case denote C₁₋₈-aliphatic residue, unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, C₁, OH, CF₃ and O—C₁₋₄-aliphatic residue, or in each case denote C₃₋₆-cycloaliphatic residue or 3 to 7 membered heterocycloaliphatic residue, in each case unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue, wherein the C₁₋₄-aliphatic residue in each case may be unsubstituted or mono- or polysubstituted with at least one substituent selected from the group consisting of F, Cl, OH, OCF₃, CF₃ and O—C₁₋₄-aliphatic residue, and wherein the C₃₋₆-cycloaliphatic residue or the 3 to 7 membered heterocycloaliphatic residue in each case may be linked via an unsubstituted C₁₋₄-aliphatic group, on the condition that if R⁵ or R⁶ denotes 3 to 7 membered heterocycloaliphatic residue, the 3 to 7 membered heterocycloaliphatic residue is linked via a carbon atom, or R³ denotes N(R⁷R⁸), wherein R⁷ denotes C₁₋₈-aliphatic residue, unsubstituted or mono- or polysubstituted with at least one substituent selected from the group consisting of F, Cl, OH, ═O, O—C₁₋₄-aliphatic residue, OCF₃, SCF₃, CF₃ and C₁₋₄-aliphatic residue wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, OH, —O, O—C₁₋₄-aliphatic residue, OCF₃, SCF₃, C(═O)—O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, OH, OCF₃, CF₃ and O—C₁₋₄-aliphatic residue, and wherein the C₃₋₆-cycloaliphatic residue or the 3 to 7 membered heterocycloaliphatic residue is in each case linked via an unsubstituted C₁₋₄-aliphatic group, on the condition that if R⁷ denotes 3 to 7 membered heterocycloaliphatic residue, the 3 to 7 membered heterocycloaliphatic residue is linked via a carbon atom, and R⁸ denotes unsubstituted C₁₋₄-aliphatic residue, or R⁷ and R⁸ form together with the nitrogen atom connecting them a 3 to 7 membered heterocycloaliphatic residue, unsubstituted or mono- or polysubstituted with at least one substituent selected from the group consisting of F, Cl, OH, ═O, O—C₁₋₄-aliphatic residue, OCF₃, SCF₃, CF₃, CN and C₁₋₄-aliphatic residue, wherein the C₁₋₄-aliphatic residue in each case may be unsubstituted or mono- or polysubstituted with at least one substituent selected from the group consisting of F, Cl, OH, CF₃ and O—C₁₋₄-aliphatic residue, and wherein the 3 to 7 membered heterocycloaliphatic residue formed by R⁷ and R⁸ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, OH, O—C₁₋₄-aliphatic residue, OCF₃, SCF₃, CF₃, CN, C₁₋₄-aliphatic residue, C(═O)OH, benzyl, phenyl, and pyridyl, wherein the C₁₋₄-aliphatic residue in each case may be unsubstituted or mono- or polysubstituted with at least one substituent selected from the group consisting of F, Cl, OH, and O—C₁₋₄-aliphatic residue, and wherein benzyl, phenyl, and pyridyl, may in each case may be unsubstituted or mono- or polysubstituted with at least one substituent selected from the group consisting of F, Cl, OCH₃, OCF₃, OCH₂CH₂OH, O CH₂CH₂OCH₃, SH, SCF₃, CF₃ and C₁₋₄-aliphatic residue; and each R⁴ independently represents H, F, Cl, Br, CN, CF₃, OCF₃, CH₃, OCH₃ or S(═O)₂CH₃.

In further embodiments, the Kv7 channel activator is selected from the group consisting of: 1 2-Ethylsulfanyl-N-[(3-fluorophenyl)-methyl]-4-methyl-[1,5]naphthyridine-3-carboxylic acid amide; 2 2-Ethylsulfanyl-N-[(4-fluorophenyl)-methyl]-4-methyl-[1,5]naphthyridine-3-carboxylic acid amide; 3-Ethylsulfanyl-N-[(3-fluorophenyl)-methyl]-4-methyl-[1,6]naphthyridine-3-carboxylic acid amide; 4 2-Ethylsulfanyl-N-[(4-fluorophenyl)-methyl]-4-methyl-[1,6]naphthyridine-3-carboxylic acid amide; 5 2-Ethylsulfanyl-N-[(3-fluorophenyl)-methyl]-4-methyl-[1,7]naphthyridine-3-carboxylic acid amide; 6-Ethylsulfanyl-N-[(4-fluorophenyl)-methyl]-4-methyl-[1,7]naphthyridine-3-carboxylic acid amide; 7 2-Ethylsulfanyl-N-[(3-fluorophenyl)-methyl]-4-methyl-[1,8]naphthyridine-3-carboxylic acid amide; 8 2-Ethylsulfanyl-N-[(4-fluorophenyl)-methyl]-4-methyl-[1,8]naphthyridine-3-carboxylic acid amide; 9 2-Ethylsulfanyl-N-[(3-fluorophenyl)-methyl]-4-methyl-7-(trifluoromethyl)-[1,8]naphthyridine-3-carboxylic acid amide; 10 2-Ethylsulfanyl-N-[(4-fluorophenyl)-methyl]-4-methyl-7-(trifluoromethyl)-[1,8]naphthyridine-3-carboxylic acid amide; 11 2-Ethylsulfanyl-N-[(3-fluorophenyl)-methyl]-4-methyl-7-(trifluoromethyl)-[1,6]naphthyridine-3-carboxylic acid amide; 12 2-Ethylsulfanyl-N-[(4-fluorophenyl)-methyl]-4-methyl-7-(trifluoromethyl)-[1,6]naphthyridine-3-carboxylic acid amide; 13 2-Ethylsulfanyl-N-[(3-fluorophenyl)-methyl]-4-methyl-7-(trifluoromethyl)-[1,5]naphthyridine-3-carboxylic acid amide; 14 2-Ethylsulfanyl-N-[(4-fluorophenyl)-methyl]-4-methyl-7-(trifluoromethyl)-[1,5]naphthyridine-3-carboxylic acid amide; 15 5-Ethylsulfanyl-N-[(3-fluorophenyl)-methyl]-7-methyl-thieno[3,2-b]pyridine-6-carboxylic acid amide; 16 6-Ethylsulfanyl-N-[(3-fluorophenyl)-methyl]-4-methyl-thieno[2,3-b]pyridine-5-carboxylic acid amide; 17 5-Ethoxy-N-[(3-fluorophenyl)-methyl]-7-methyl-2-(trifluoromethyl)-thieno[3,2-b]pyridine-6-carboxylic acid amide; 18 6-Ethoxy-N-[(3-fluorophenyl)-methyl]-4-methyl-2-(trifluoromethyl)-thieno[2,3-b]pyridine-5-carboxylic acid amide; in the form of a free compound, a solvate and/or a physiologically acceptable salt.

### Formula 20

In an embodiment, the Kv7 channel activator may be selected from one of the following compounds according to Formula 20. Such compounds are described in U.S. Pat. No. 9,284,286 issued Mar. 15, 2016 and corresponding to U.S. application Ser. No. 14/091,373 filed Nov. 27, 2013; International Publication No. WO2014082737A1, published Jun. 5, 2014 and corresponding to International Application No. PCT/EP2013/003572 filed Nov. 27, 2013; which are incorporated by reference in their entirety herein. In the case of any conflict of terminology in the context of Formula 20, these references incorporated by reference herein control.

In an embodiment, the Kv7 channel activator is a compound according to formula 20:

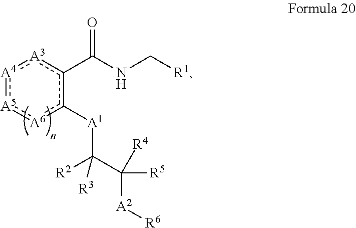

wherein: A¹ represents CR¹⁰R¹¹ or S; A² represents CR¹²R¹³, C(═O), O, S, S(═O) or S(═O)₂; A³, A⁴ and A⁵ independently of each other represent CR⁷, N, O, S or NR⁸, A⁶ represents CR⁷ or N, and n denotes 0 or 1, with the proviso, that if n denotes 0, then precisely one of A³, A⁴ and A⁵ represents O, S or NR⁸, or if n denotes 1, then A³, A⁴ and A⁵ independently of each other represent CR⁷ or N; and with the proviso, that if n denotes 1 and A³, A⁴ and A⁵ each represent CR⁷, then A⁶ does not represent N; R¹ represents C₁₋₁₀-aliphatic residue, unsubstituted or mono- or polysubstituted; C₃₋₁₀-cycloaliphatic residue or a 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted; aryl or heteroaryl, in each case unsubstituted or mono- or polysubstituted and in each case optionally linked via a C₁₋₄-aliphatic group, which in turn may be unsubstituted or mono- or polysubstituted; R², R³; R⁴, R⁵, R¹⁰, R¹¹, R¹² and R¹³ each independently of another represent H; F; Cl; Br; I; NO₂; CF₃; CN; OH; OCF₃; SH; SCF₃; C₁₋₁₀-aliphatic residue, O—C₁₋₁₀-aliphatic residue or S—C₁₋₁₀-aliphatic residue, in each case saturated or unsaturated, branched or unbranched, unsubstituted or mono- or poly-substituted; or C₃₋₁₀-cycloaliphatic residue, saturated or unsaturated, branched or unbranched, unsubstituted or mono- or poly-substituted; or R² and R³ or R⁴ and R⁵ or R¹⁰ and R¹¹ or R¹² and R¹³ or R² and R¹¹ or R² and R⁴ or R² and R¹³ or R⁴ and R¹³ or R⁴ and R¹¹ or R¹² and R¹³, together with the carbon atom(s) joining them, form a C₃₋₁₀-cycloaliphatic residue or a 3 to 10 membered heterocycloaliphatic residue, in each case saturated or unsaturated, unsubstituted or mono- or poly-substituted; wherein the remaining substituents R², R³; R⁴; R⁵; R¹⁰, R¹¹, R¹² and R¹³ in each case have the meaning given above; R⁶ represents a C₃₋₁₀-cycloaliphatic residue or a 3 to 10 membered heterocycloaliphatic residue, in each case saturated or unsaturated, unsubstituted or mono- or poly-substituted; or represents an aryl or a heteroaryl, in each case unsubstituted or mono- or poly-substituted; each R⁷ independently of each other represents H, F; Cl; CN; CF₃; CHF₂; CH₂F; OCF₃; OCHF₂; OCH₂F; SCF₃; O—C₁₋₄-aliphatic residue, C₁₋₄-aliphatic residue or S(═O)₂-C₁₋₄-aliphatic residue, wherein the C₁₋₄-aliphatic residue in each case may be saturated or unsaturated, branched or unbranched, unsubstituted or mono- or poly-substituted; and R⁸ represents H or a C₁₋₄-aliphatic residue, wherein the aliphatic residue may be saturated or unsaturated, branched or unbranched, unsubstituted or mono- or poly-substituted; in which an “aliphatic group” and “aliphatic residue” may in each case be branched or unbranched, saturated or unsaturated, in which a “cycloaliphatic residue” and a “heterocycloaliphatic residue” may in each case be saturated or unsaturated, in which “mono- or polysubstituted” with respect to an “aliphatic group”, an “aliphatic residue”, a “cycloaliphatic residue” and a “heterocycloaliphatic residue” relates, with respect to the corresponding residues or groups, to the substitution of one or more hydrogen atoms each independently of one another by at least one substituent selected from the group consisting of F, Cl, Br, I, NO₂, NH₂, NH(C₁₋₄-aliphatic residue), N(C₁₋₄-aliphatic residue)₂, NH—C(═O)-C₁₋₄aliphatic residue, N(C₁₋₄-aliphatic residue)-C(═O)—C₁₋₄-aliphatic residue, NH—S(═O)₂-C₁₋₄-aliphatic residue, N(C₁₋₄-aliphatic residue)-S(═O)₂-C₁₋₄-aliphatic residue, ═O, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂-C₁₋₄-aliphatic residue, S(═O)₂-O—C₁₋₄-aliphatic residue, S(═O)₂—NH(C₁₋₄-aliphatic residue), S(═O)₂—N(C₁₋₄-aliphatic residue)₂, CN, CF₃, CHO, COOH, C₁₋₄-aliphatic residue, C(═O)-C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, a C(═O)—NH(C₁₋₄-aliphatic residue) and C(═O)—N(C₁₋₄-aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

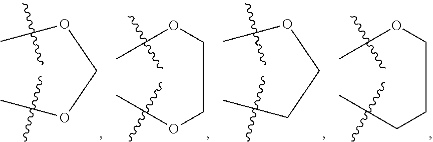 [SMILES: *OCO* ; *OCCO* ; *CCO* ; *CCCO*]

NH(C₁₋₄-aliphatic residue), N(C₁₋₄-aliphatic residue)₂, NH—C(═O)—C₁₋₄-aliphatic residue, N(C₁₋₄ aliphatic residue)-C(═O)—C₁₋₄ aliphatic residue, NH—S(═O)₂-C₁₋₄ aliphatic residue, N(C₁₋₄ aliphatic residue)-S(═O)₂-C₁₋₄ aliphatic residue, OH, OCF₃, O—C₁₋₄-aliphatic residue, O—C(═O)-C₁₋₄-aliphatic residue, SH, SCF₃, S—C₁₋₄-aliphatic residue, S(═O)₂OH, S(═O)₂-C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, C(═O) H, C(═O) OH, C₁₋₄-aliphatic residue, C(═O)-C₁₋₄-aliphatic residue, C(O)—O—C₁₋₄-aliphatic residue, C₃₋₆-cycloaliphatic residue, 3 to 7 membered heterocycloaliphatic residue, benzyl, aryl, heteroaryl, C(═O)NH₂, C(═O)—NH(C₁₋₄-aliphatic residue) and C(═O)—N(C₁₋₄-aliphatic residue)₂; in the form of an individual single stereoisomer or a mixture of the stereoisomers in any mixing ratio, and/or in the form of a free compound, a solvate and/or a physiologically acceptable salt.

In further embodiments, A¹ represents CR¹⁰R¹¹ or S; A² represents CR¹²R¹³, C(═O), O, S, S(═O) or S(═O)₂; A³, A⁴ and A⁵ independently of each other represent CR⁷, N, O, S or NR⁸, A⁶ represents CR⁷ or N, and n denotes 0 or 1, with the proviso, that if n denotes 0, then precisely one of A³, A⁴ and A⁵ represents O, S or NR⁸, or if n denotes 1, then A³, A⁴ and A⁵ independently of each other represent CR⁷ or N; and with the proviso, that if n denotes 1 and A³, A⁴ and A⁵ each represent CR⁷, then A⁶ does not represent N; R¹ denotes a C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue, C(═O) OH, C₃₋₆-cycloaliphatic residue and a 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the C₃₋₁₀-cycloaliphatic residue or the 3 to 10 membered heterocycloaliphatic residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, or denotes an aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, C₃₋₆ cycloaliphatic residue, 3 to 6 membered

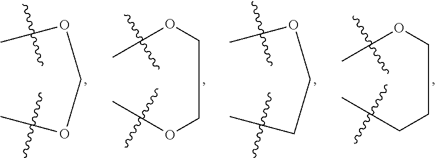 [SMILES: *OCO* ; *OCCO* ; *CCO* ; *CCCO*]

heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S-C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O) OH, R², R³, R⁵, R¹⁰, R¹¹, R¹² and R¹³ each independently of the others represents H; F; Cl; Br; I; NO₂; CF₃; CN; OH; OCF₃; SH; SCF₃; C₁₋₄-aliphatic residue, O—C₁₋₄-aliphatic residue or S—C₁₋₄-aliphatic residue, in each case saturated or unsaturated, branched or unbranched, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, or a C₃₋₁₀-cycloaliphatic residue, saturated or unsaturated, branched or unbranched, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₁₀-cycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, or R² and R³ or R⁴ and R⁵ or R¹⁰ and R¹¹ or R¹² and R¹³ or R² and R¹¹ or R² and R⁴ or R² and R¹³ or R⁴ and R¹³ or R⁴ and R¹¹ or R¹² and R¹³, together with the carbon atom(s) joining them, form a C₃₋₁₀-cycloaliphatic residue or a 3 to 10 membered heterocycloaliphatic residue, in each case saturated or unsaturated and in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)OH, and wherein the remaining substituents R², R³, R⁴, R⁵, R¹⁰, R¹¹, R¹² and R¹³ in each case have the meaning given above; R⁶ represents a C₃₋₁₀-cycloaliphatic residue or a 3 to 10 membered heterocycloaliphatic residue, in each case saturated or unsaturated and in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O) OH, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(O) OH, and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, or represents an aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

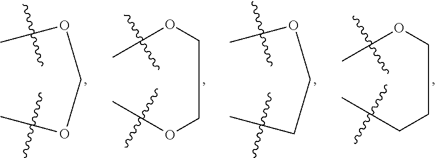 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN and C(═O) OH, each R⁷ independently of each other represents H, F; Cl; CN; CF₃; CHF₂; CH₂F; OCF₃; OCHF₂; OCH₂F; SCF₃; a O—C₁₋₄-aliphatic residue, a C₁₋₄-aliphatic residue or a S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and O—C₁₋₄-aliphatic residue, and R⁸ represents H or C₁₋₄-aliphatic residue, wherein the C₁₋₄-aliphatic residue may be unsubstituted or mono- or polysubstituted with at least one substituent selected from the group consisting of F, Cl, Br, I, OH, OCF₃, CF₃ and O—C₁₋₄-aliphatic residue.

In further embodiments, A¹ represents S; and A² represents S, S(═O)₂ or CR¹²R¹³ wherein R¹² and R¹³ both represent H or both represent F.

In further embodiments, n denotes 1 and A³ represents CR⁷, A⁴ represents CR⁷, A⁵ represents CR⁷ and A⁶ represents CR⁷; or n denotes 1 and A³ represents N, A⁴ represents CR⁷, A⁵ represents CR⁷ and A⁶ represents CR⁷; or n denotes 1 and A³ represents CR⁷, A⁴ represents N, A⁵ represents CR⁷ and A⁶ represents CR⁷; or n denotes 1 and A³ represents CR⁷, A⁴ represents CR⁷, A⁵ represents N and A⁶ represents CR⁷; or n denotes 1 and A³ represents N, A⁴ represents N, A⁵ represents CR⁷ and A⁶ represents CR⁷; or n denotes 1 and A³ represents N, A⁴ represents CR⁷, A⁵ represents N and A⁶ represents CR⁷; or n denotes 1 and A³ represents N, A⁴ represents CR⁷, A⁵ represents CR⁷ and A⁶ represents N; or n denotes 1 and A³ represents CR⁷, A⁴ represents N, A⁵ represents CR⁷ and A⁶ represents N; or n denotes 1 and A³ represents CR⁷, A⁴ represents N, A⁵ represents N and A⁶ represents CR⁷; or n denotes 1 and A³ represents CR⁷, A⁴ represents CR⁷, A⁵ represents N and A⁶ represents N; or n denotes 0 and A³ represents S, A⁴ represents CR⁷ and A⁵ represents CR⁷; or n denotes 0 and A³ represents S, A⁴ represents CR⁷ and A⁵ represents N; or n denotes 0 and A³ represents O, A⁴ represents CR⁷ and A⁵ represents CR⁷, or n denotes 0 and A³ represents O, A⁴ represents CR⁷ and A⁵ represents N; or n denotes 0 and A³ represents CR⁷, A⁴ represents CR⁷ and A⁵ represents S; or n denotes 0 and A³ represents N, A⁴ represents CR⁷ and A⁵ represents S; or n denotes 0 and A³ represents CR⁷, A⁴ represents CR⁷ and A⁵ represents O; or n denotes 0 and A³ represents N, A⁴ represents CR⁷ and A⁵ represents O.

In further embodiments, R¹ represents the partial structure:

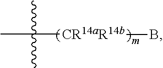

wherein: R^{14a} and R^{14b} each independently of the other represent H; F; Cl; Br; CF₃; CN; OH; OCF₃; NH₂; C₁₋₄-aliphatic residue, O—C₁₋₄-aliphatic residue, NH(C₁₋₄-aliphatic residue), N(C₁₋₄-aliphatic residue)₂, in each case saturated or unsaturated, branched or unbranched, unsubstituted or mono- or poly-substituted by one or more substituents each selected independently of one another from the group consisting of F, Cl, Br, I, O—C₁₋₄-aliphatic residue, OH and OCF₃; or independently represent C₃₋₁₀-cycloaliphatic residue or 3 to 10 membered heterocycloaliphatic residue, in each case saturated or unsaturated, unsubstituted or mono- or poly-substituted by one or more substituents each selected independently of one another from the group consisting of F, Cl, Br, C₁₋₄-aliphatic residue, OH, ═O, O—C₁₋₄-aliphatic residue, OCF₃, NH₂, NH(C₁₋₄-aliphatic residue) and N(C₁₋₄-aliphatic residue)₂; m represents 0, 1, 2 or 3; Y represents O or NR¹⁵, wherein R¹⁵ represents H or C₁₋₄-aliphatic residue, saturated or unsaturated, branched or unbranched, unsubstituted or mono- or poly-substituted by one or more substituents each selected independently of one another from the group consisting of F, Cl, Br, C₁₋₄-aliphatic residue, OH, O—C₁₋₄-aliphatic residue, OCF₃, NH₂, NH(C₁₋₄-aliphatic residue) and N(C₁₋₄-aliphatic residue)₂; or C₃₋₁₀-cycloaliphatic residue, saturated or unsaturated, unsubstituted or mono- or poly-substituted by one or more substituents each selected independently of one another from the group consisting of F, Cl, Br, C₁₋₄-aliphatic residue, OH, O—C₁₋₄-aliphatic residue, OCF₃, NH₂, NH(C₁₋₄-aliphatic residue) and N(C₁₋₄-aliphatic residue)₂; o represents 0 or 1, B represents C₁₋₈-aliphatic residue, saturated or unsaturated, branched or unbranched, unsubstituted or mono- or poly-substituted by one or more substituents each selected independently of one another from the group consisting of F, Cl, Br, CN, OH, ═O, O—C₁₋₄-aliphatic residue, OCF₃, C(═O)OH, CF₃, NH₂, NH(C₁₋₄-aliphatic residue) and N(C₁₋₄-aliphatic residue)₂; or C₃₋₁₀-cycloaliphatic residue or 3 to 10 membered heterocycloaliphatic residue, in each case saturated or unsaturated, unsubstituted or mono- or poly-substituted by one or more substituents each selected independently of one another from the group consisting of F, Cl, Br, CN, OH, O—C₁₋₄-aliphatic residue, OCF₃, C₁₋₄-aliphatic residue, C(═O)—OH, CF₃, NH₂, NH(C₁₋₄-aliphatic residue), N(C₁₋₄-aliphatic residue)₂ and SCF₃; or aryl or heteroaryl, in each case unsubstituted or mono- or poly-substituted by one or more substituents each selected independently of one another from the group consisting of F, Cl, Br, NO₂, CN, OH, O—C₁₋₄-aliphatic residue, OCF₃, C₁₋₄-aliphatic residue, C(═O) OH, CF₃, NH₂, NH(C₁₋₄-aliphatic residue), N(C₁₋₄-aliphatic residue)₂, S—C₁₋₄-aliphatic residue, SCF₃, benzyl, phenyl, pyridyl and thienyl, wherein benzyl, phenyl, pyridyl, thienyl can in each case be unsubstituted or mono- or poly-substituted by one or more substituents selected independently of one another from the group consisting of F, Cl, Br, NO₂, CN, OH, O—C₁₋₄-aliphatic residue, OCF₃, C₁₋₄-aliphatic residue, C(═O)—OH, CF₃, NH₂, NH(C₁₋₄-aliphatic residue), N(C₁₋₄-aliphatic residue)₂, S—C₁₋₄-aliphatic residue and SCF₃.

In further embodiments, R^{14a} and R^{14b} each independently of the other represents H; F; Cl; CH₃; CH₂CH₃; (CH₂)₂CH₃; CH(CH₃)₂; (CH₂)₃CH₃; CH(CH)₃CH₂CH₃; C(CH₃)₃; OH; OCH₃; OCH₂CH₃; O(CH₂)₂OCH₃ or O(CH₂)₂OH; m represents 0, 1 or 2 and represents 0 and B represents CH₃; CH₂CH₃; (CH₂)₂CH₃; CH(CH₃)₂; (CH₂)₃CH₃; CH(CH₃) CH₂CH₃; C(CH₃)₃; cyclopropyl; cyclobutyl; cyclopentyl; cyclohexyl; cycloheptyl; adamantyl; bicyclo[2.2.1]heptyl; bicyclo[2.2.2]octyl; phenyl, pyridyl or thienyl, in each case unsubstituted or mono-, di- or tri-substituted by one, two or three substituents each selected independently of one another from the group consisting of F, Cl, CN, OH, O—C₁₋₄-aliphatic residue, OCF₃, C₁₋₄-aliphatic residue, CF₃, NH₂, NH(C₁₋₄-aliphatic residue) and N(C₁₋₄-aliphatic residue)₂.

In further embodiments, R²; R³; R⁴; R⁵; R¹⁰, R¹¹, R¹² and R¹³ each independently of the others represents H; F; Cl; CF₃; CN; OH; OCF₃; SCF₃; CH₃; CH₂CH₃; (CH₂)₂CH₃; CH(CH₃)₂; (CH₂)₃CH₃; CH(CH)₃CH₂CH₃; C(CH₃)₃; OCH₃; OCH₂CH₃; O(CH₂)₂OCH₃; O(CH₂)₂OH; SCH₃; SCH₂CH₃; cyclopropyl; cyclobutyl; cyclopentyl; cyclohexyl; or R² and R³ or R⁴ and R⁵ or R¹⁰ and R¹¹ or R¹² and R¹³ or R² and R¹¹ or R² and R⁴ or R² and R¹³ or R⁴ and R¹³ or R⁴ and R¹¹ or R¹² and R¹³, together with the carbon atom(s) joining them, form a cyclopropyl, cyclobutyl, cyclopentyl or cyclohexyl, in each case unsubstituted; wherein the remaining substituents R², R³; R⁴; R⁵; R¹⁰, R¹¹, R¹² and R¹³ in each case have the meaning given above.

In further embodiments, each R⁷ represents H, F; Cl; CN; CF₃; CHF₂; CH₂F; OCF₃; OCHF₂; OCH₂F; SCF₃; CH₃; CH₂CH₃; CH₂CH₂CH₃; CH(CH₃)₂; OH₂CH₂CH₂CH₃; CH(CH)₃CH₂CH₃; CH₂CH(CH₃)₂; O(CH₃)₃; OCH₃; OCH₂CH₃; O(CH₂)₂OCH₃; O(CH₂)₂OH; S(═O)₂CH₃ S(═O)₂CH₂CH₃ S(═O)₂CH(CH₃)₂ or S(═O)₂CH₂CH₂CH₃; and R⁸ represents H or CH₃ or CH₂CH₃ or CH(CH₃)₂.

In further embodiments, R⁶ represents a C₃₋₁₀-cycloaliphatic residue or a 3 to 10 membered heterocycloaliphatic residue, in each case saturated or unsaturated, unsubstituted or mono- or poly-substituted by one or more substituents each selected independently of one another from the group consisting of F, Cl, Br, I, NO₂, CN, OH, —O, O—C₁₋₄-aliphatic residue, OCF₃, C₁₋₄-aliphatic residue, CF₃, SH, S—C₁₋₄-aliphatic residue and SCF₃; or an aryl or a heteroaryl, in each case unsubstituted or mono- or poly-substituted by one or more substituents each selected independently of one another from the group consisting of F, Cl, Br, I, NO₂, CN, OH, O—C₁₋₄-aliphatic residue, OCF₃, C₁₋₄-aliphatic residue, CF₃, NH₂, NH(C₁₋₄-aliphatic residue), N(C₁₋₄-aliphatic residue)₂, SH, S—C₁₋₄-aliphatic residue and SCF₃.

In further embodiments, R⁶ represents phenyl, pyridyl or thienyl, in each case unsubstituted or mono- or poly-substituted by one or more substituents each selected independently of one another from the group consisting of F, Cl, Br, CN, OH, O—C₁₋₄-aliphatic residue, OCF₃, C₁₋₄-aliphatic residue, CF₃ and SCF₃.

In further embodiments, A¹ represents S; and A² represents S, S(═O)₂ or CR¹²R¹³ wherein R¹² and R¹³ both represent H or both represent F; and R¹ represents the partial structure:

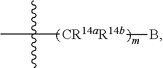

wherein: R^{14a} and R^{14b} each independently of the other represents H; F; Cl; CH₃; CH₂CH₃; (CH₂)₂CH₃; CH(CH₃)₂; (OH₂)₃CH₃; CH(CH)₃CH₂CH₃; C(CH₃)₃; OH; OCH₃; OCH₂CH₃; O(CH₂)₂OCH₃; or O(CH₂)₂OH; m represents 0, 1 or 2 and B represents phenyl or naphthyl or pyridyl or thienyl, in each case unsubstituted or mono- or di- or tri-substituted by one, two or three substituents each selected independently of one another from the group consisting of F, C₁, CN, OH, O-₁₋₄-aliphatic residue, OCF₃, C₁₋₄-aliphatic residue, C(═O)—OH, CF₃, NH₂, NH(C₁₋₄-aliphatic residue) and N(C₁₋₄-aliphatic residue)₂; R², R³, R⁴; R⁵, R¹⁰, R¹¹, R¹² and R¹³ each independently of the others represent H; F; Cl; Br; I; NO₂; CF₃; CN; OH; OCF₃; SH; SCF₃; CH₃; CH₂CH₃; CH₂CH₂CH₃; CH(CH₃)₂; CH₂CH₂CH₂CH₃; CH(CH)₃CH₂CH₃; CH₂CH(CH₃)₂; C(CH₃)₃; OCH₃; OCH₂CH₃; O(CH₂)₂OCH₃; O(CH₂)₂OH; SCH₃; SCH₂CH₃; cyclopropyl; cyclobutyl; cyclopentyl; cyclohexyl; or R² and R³ or R⁴ and R⁵ or R¹⁰ and R¹¹ or R¹² and R¹³ or R² and R¹¹ or R² and R⁴ or R² and R¹³ or R⁴ and R¹³ or R⁴ and R¹¹ or R¹² and R¹³ together with the carbon atom(s) joining them, form a cyclopropyl, cyclobutyl, cyclopentyl or cyclohexyl, in each case unsubstituted; wherein the remaining substituents R², R³; R⁴; R⁵; R¹⁰, R¹¹, R¹² and R¹³ in each case have the meaning given above; R⁶ represents phenyl, in each case unsubstituted or mono- or poly-substituted by one or more substituents each selected independently of one another from the group consisting of F, Cl, Br, I, CN, OH, OCH₃, OCH₂CH₃, OCF₃, CH₃, CH₂CH₃, CH(CH₃)₂ and CF₃; each R⁷ represents H, F; Cl; CN; CF₃; CHF₂; CH₂F; OCF₃; OCHF₂; OCH₂F; SCF₃; CH₃; CH₂CH₃; CH₂CH₂CH₃; CH(CH₃)₂; CH₂CH₂CH₂CH₃; CH(CH)₃CH₂CH₃; CH₂CH(CH₃)₂; C(CH₃)₃; OCH₃; OCH₂CH₃; O(CH₂)₂OCH₃; O(CH₂)₂OH; S(═O)₂CH₃ S(═O)₂CH₂CH₃, S(═O)₂CH(CH₃)₂ or S(═O)₂CH₂CH₂CH₃; and R⁸ represents H or CH₃ or CH₂CH₃ or CH(CH₃)₂.

In further embodiments, the Kv7 channel activator is selected from the group consisting of: 1 2-[[3,3-Difluoro-3-(4-fluorophenyl)-propyl]sulfanyl]-N-(3-methyl-butyl)-benzamide; 2 N-(3,3-Dimethyl-butyl)-2-[3-(4-fluorophenyl)-propylsulfanyl]-benzamide; 3 3-[[3,3-Difluoro-3-(4-fluorophenyl)-propyl]sulfanyl]-N-(3-methyl-butyl)-pyridine-2-carboxylic acid amide; 4 3-[2-(Benzenesulfonyl)-ethylsulfanyl]-N-(thiophen-2-yl-methyl)-pyridine-2-carboxylic acid amide; 5 4-[[3,3-Difluoro-3-(4-fluorophenyl)-propyl]sulfanyl]-N-(3-methyl-butyl)-pyridine-3-carboxylic acid amide; 6 4-[[3,3-Difluoro-3-(4-fluorophenyl)-propyl]sulfanyl]-N-[(4-fluorophenyl)-methyl]-pyridine-3-carboxylic acid amide; 7 3-[[3,3-Difluoro-3-(4-fluorophenyl)-propyl]sulfanyl]-N-(3-methyl-butyl)-pyridine-4-carboxylic acid amide; 8 3-[2-(Benzenesulfonyl)-ethylsulfanyl]-N-(thiophen-2-yl-methyl)-pyridine-4-carboxylic acid amide; 9 3-[[3,3-Difluoro-3-(4-fluorophenyl)-propyl]sulfanyl]-N-(3-methyl-butyl)-pyrazine-2-carboxylic acid amide; 10 4-[[3,3-Difluoro-3-(4-fluorophenyl)-propyl]sulfanyl]-N-(3-methyl-butyl)-pyrimidine-5-carboxylic acid amide; 11 4-[[3,3-Difluoro-3-(4-fluorophenyl)-propyl]sulfanyl]-N-[(4-fluorophenyl)-methyl]-pyrimidine-5-carboxylic acid amide; 12 3-[[3,3-Difluoro-3-(4-fluorophenyl)-propyl]sulfanyl]-N-(3-methyl-butyl)-pyridazine-4-carboxylic acid amide; 13 3-[[3,3-Difluoro-3-(4-fluorophenyl)-propyl]sulfanyl]-N-[(4-fluorophenyl)-methyl]-pyridazine-4-carboxylic acid amide; 14 4-[[3,3-Difluoro-3-(4-fluorophenyl)-propyl]sulfanyl]-N-[(4-fluorophenyl)-methyl]-thiazole-5-carboxylic acid amide; 15 4-[[3,3-Difluoro-3-(4-fluorophenyl)-propyl]sulfanyl]-N-[(4-fluorophenyl)-methyl]-2-methyl-thiazole-5-carboxylic acid amide; in the form of a free compound, a solvate and/or a physiologically acceptable salt.

### Formula 21

In another embodiment, the Kv7 channel activator may be selected from one of the following compounds. Such compounds are described in US Publication No.

US20140148454A1, published May 29, 2014 and corresponding to U.S. application Ser. No. 14/091,378 filed Nov. 27, 2013; International Publication No. WO2014082739A1, published Jun. 5, 2014 and corresponding to International Application No. PCT/EP2013/003574 filed Nov. 27, 2013; which are incorporated by reference in their entirety herein. In the case of any conflict of terminology in the context of Formula 21, these references incorporated by reference herein control.

In an embodiment, the Kv7 channel activator is a compound according to formula 21:

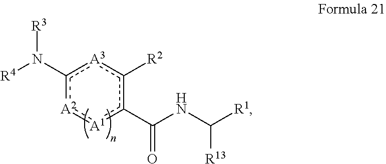

wherein, A¹ represents CR⁵ or N; A² represents CR⁶, N, O, S or NR⁷; A³ represents CR⁸ or N, and n denotes 0 or 1, on the condition, that if n denotes 0, then A² represents O, S or NR⁷, or if n denotes 1, then A² represents CR⁶ or N, wherein R⁵ is selected from F, Cl, Br, CN, CH₃, CF₃, CHF₂, CH₂F, OCH₃, C₂H₅, SCH₃, OCF₃, OCHF₂ or OCH₂F; R⁶ is selected from H, F, Cl, Br, CN, CH₃, CF₃, CHF₂, CH₂F, OCH₃, C₂H₅, SCH₃, OCF₃, OCHF₂ or OCH₂F; R⁷ represents C₁₋₄-aliphatic residue or C₃₋₅-cycloaliphatic residue, in each case unsubstituted or mono- or polysubstituted; R⁸ is selected from H, F, Cl, Br, CN, CH₃, CF₃, CHF₂, CH₂F, OCH₃, C₂H₅, SCH₃, OCF₃, OCHF₂ or OCH₂F; with the proviso, that, if n denotes 1, then at least one of A¹, A² and A³ denotes N, with the proviso, that if n denotes 1 and A³ denotes N, then A¹ and/or A² denotes N, and with the proviso, that if n denotes 1 and A² denotes N and A¹ denotes CR⁵ and A³ denotes CR⁸, then R⁵ denotes F, Cl, CH₃, CF₃, CHF₂ or CH₂F; R¹³ represents H or C₁₋₄-aliphatic residue, R¹ represents C₁₋₁₀-aliphatic residue, unsubstituted or mono- or polysubstituted; or C₃₋₁₀-cycloaliphatic residue or 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted; or aryl or heteroaryl, in each case unsubstituted or mono- or polysubstituted and in each case optionally linked via a C₁₋₄-aliphatic group, which in turn may be unsubstituted or mono- or polysubstituted; R² represents a C₁₋₆-aliphatic residue, unsubstituted or mono- or polysubstituted; a C₃₋₆-cycloaliphatic residue or a 3 to 7 membered heterocycloaliphatic residue, in each case unsubstituted or mono- or polysubstituted, or denotes S—R⁹, O—R¹⁰ or N(R¹¹R¹²), wherein R⁹ and R¹⁰ in each case represent C₁₋₆-aliphatic residue, unsubstituted or mono- or polysubstituted; C₃₋₆-cycloaliphatic residue or 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted; with the proviso, that if R⁹ or R¹⁰ denote a 3 to 7 membered heterocycloaliphatic residue, than the 3 to 7 membered heterocycloaliphatic residue is linked via a carbon atom, R¹¹ represents C₁₋₆-aliphatic residue, unsubstituted or mono- or polysubstituted; C₃₋₆-cycloaliphatic residue or a 3 to 7 membered heterocycloaliphatic residue, in each case unsubstituted or mono- or polysubstituted and in each case optionally linked via a C₁₋₄-aliphatic group, which in turn may be unsubstituted or mono- or polysubstituted; with the proviso that if R¹¹ denotes a 3 to 7 membered heterocycloaliphatic residue, the 3 to 7 membered heterocycloaliphatic residue is linked via a carbon atom; and R¹² denotes C₁₋₆-aliphatic residue, unsubstituted or mono- or polysubstituted; or R¹¹ and R¹² form together with the nitrogen atom connecting them a 3 to 7 membered heterocycloaliphatic residue, unsubstituted or mono- or polysubstituted; R³ represents C₁₋₁₀-aliphatic residue, unsubstituted or mono- or polysubstituted; or C₃₋₁₀-cycloaliphatic residue or 3 to 10 membered heterocycloaliphatic residue, in each case unsubstituted or mono- or polysubstituted and in each case optionally linked via a C₁₋₄-aliphatic group, which in turn may be unsubstituted or mono- or polysubstituted; or aryl or heteroaryl, in each case unsubstituted or mono- or polysubstituted and in each case optionally linked via a C₁₋₄-aliphatic group, which in turn may be unsubstituted or mono- or polysubstituted; on the condition that if R³ denotes a 3 to 10 membered heterocycloaliphatic residue or a heteroaryl, the 3 to 10 membered heterocycloaliphatic residue or the heteroaryl is linked via a carbon atom; and R⁴ denotes H or C₁₋₁₀-aliphatic residue, unsubstituted or mono- or polysubstituted; or R³ and R⁴ form together with the nitrogen atom connecting them a 3 to 10 membered heterocycloaliphatic residue, unsubstituted or mono- or polysubstituted; in which an “aliphatic group” and “aliphatic residue” may in each case be branched or unbranched, saturated or unsaturated, in which a “cycloaliphatic residue” and a “heterocycloaliphatic residue” may in each case be saturated or unsaturated, in which “mono- or polysubstituted” with respect to an “aliphatic group”, an “aliphatic residue”, a “cycloaliphatic residue” and a “heterocycloaliphatic residue” relates, with respect to the corresponding residues or groups, to the substitution of one or more hydrogen atoms each independently of one another by at least one substituent selected from the group consisting of F, Cl, Br, I, NO₂, NH₂, NH(C₁₋₄-aliphatic residue), N(C₁₋₄-aliphatic residue)₂, NH—C(═O)-C₁₋₄-aliphatic residue, N(C₁₋₄ aliphatic residue)-C(═O)—C₁₋₄ aliphatic residue, NH—S(═O)₂-C₁₋₄ aliphatic residue, N(C₁₋₄ aliphatic residue)-S(═O)₂-C₁₋₄ aliphatic residue, ═O, OH, OCF₃, O—C₁₋₄-aliphatic residue, O—C(═O)—C₁₋₄-aliphatic residue, SH, SCF₃, S—C₁₋₄-aliphatic residue, S(═O)₂OH, S(═O)₂-C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, CHO, COOH, C₁₋₄-aliphatic residue, C(═O)—C₁₋₄-aliphatic residue, C(—O)—O—C₁₋₄-aliphatic residue, C₃₋₆-cycloaliphatic residue, 3 to 7 membered heterocycloaliphatic residue, C(═O)NH₂, a C(═O)—NH(C₁₋₄-aliphatic residue) and C(═O)-N(C₁₋₄-aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of

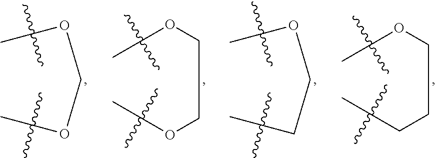 [SMILES: *OCO* ; *OCCO* ; *CCO* ; *CCCO*]

F, Cl, Br, I, NO₂, NH₂, NH(C₁₋₄-aliphatic residue), N(C₁₋₄-aliphatic residue)₂, NH—C(═O)—C₁₋₄-aliphatic residue, N(C₁₋₄ aliphatic residue)-O (═O)—C₁₋₄ aliphatic residue, NH—S(═O)₂-C₁₋₄ aliphatic residue, N(C₁₋₄ aliphatic residue)-S(═O)₂-C₁₋₄ aliphatic residue, OH, OCF₃, O—C₁₋₄-aliphatic residue, O—C(═O)-C₁₋₄-aliphatic residue, SH, SCF₃, S—C₁₋₄-aliphatic residue, S(═O)₂OH, S(═O)₂-C₁₋₄-aliphatic residue, S(═O)₂—O—C₁₋₄-aliphatic residue, S(═O)₂—NH(C₁₋₄-aliphatic residue), S(═O)₂—N(C₁₋₄-aliphatic residue)₂, CN, CF₃, C(═O) H, C(═O)OH, C₁₋₄-aliphatic residue, C(═O)—C₁₋₄-aliphatic residue, C(═O)—O—C₁₋₄-aliphatic residue, C₃₋₆-cycloaliphatic residue, 3 to 7 membered heterocycloaliphatic residue, benzyl, aryl, heteroaryl, C(═O)NH₂, C(═O)—NH(C₁₋₄-aliphatic residue) and C(═O)—N(C₁₋₄-aliphatic residue)₂; in the form of an individual single stereoisomer or a mixture of the stereoisomers in any mixing ratio, and/or in the form of a free compound, a solvate and/or a physiologically acceptable salt.

In further embodiments, A¹ represents CR⁵ or N; A² represents CR⁶, N, O, S or NR⁷; A³ represents CR⁸ or N, and n denotes 0 or 1, on the condition, that if n denotes 0, then A² represents O, S or NR⁷, or if n denotes 1, then A² represents CR⁶ or N, wherein R⁵ is selected from F, Cl, Br, CN, CH₃, CF₃, CHF₂, CH₂F, OCH₃, C₂H₅, SCH₃, OCF₃, OCHF₂ or OCH₂F; R⁶ is selected from H, F, Cl, Br, CN, CH₃, CF₃, CHF₂, CH₂F, OCH₃, C₂H₅, SCH₃, OCF₃, OCHF₂ or OCH₂F; R⁷ represents C₁₋₄-aliphatic residue or C₃₋₅-cycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, R⁸ is selected from H, F, Cl, Br, CN, CH₃, CF₃, CHF₂, CH₂F, OCH₃, C₂H₅, SCH₃, OCF₃, OCHF₂ or OCH₂F; with the proviso, that, if n denotes 1, then at least one of A¹, A² and A³ denotes N, with the proviso, that if n denotes 1 and A³ denotes N, then A¹ and/or A² denotes N, and with the proviso, that if n denotes 1 and A² denotes N and A¹ denotes CR⁵ and A³ denotes CR⁸, then R⁵ denotes F, Cl, CH₃, CF₃, CHF₂ or CH₂F; R¹³ represents H or C₁₋₄-aliphatic residue, R¹ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O) OH, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O)—OH, and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄ aliphatic residue, OCF₃, SH, SCF₃, S—C₁₋₄-aliphatic residue, CF₃, CN, C₁₋₄-aliphatic residue and C(═O) OH, or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

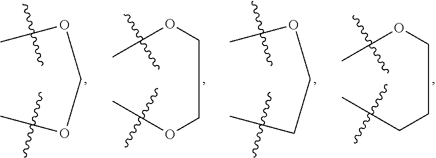 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, a C₁₋₄-aliphatic residue, C(═O) OH, C(═O)CH₃, C(═O)C₂H₅, C(═O)—OCH₃ and C(═O)—OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, an NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I,

NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O) OH, R² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH, or denotes S—R⁹, O—R¹⁰ or N(R¹¹R¹²); wherein R⁹ and R¹⁰ in each case represent C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, or in each case represent C₃₋₆-cycloaliphatic residue or 3 to 7 membered heterocyclo-aliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O) OH, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, on the condition that if R⁹ or R¹⁰ denote a 3 to 7 membered heterocycloaliphatic residue, the 3 to 7 membered heterocycloaliphatic residue is linked via a carbon atom, R¹¹ denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue, and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, or denotes C₃₋₆-cycloaliphatic residue or 3 to 7 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue,

C(═O) OH, C(═O)—O—C₁₋₄-aliphatic residue, C₃₋₆-cycloaliphatic residue, and a 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃,

S—C₁₋₄-aliphatic residue, CF₃, CN, C₁₋₄-aliphatic residue and C(═O)—OH, and wherein the C₃₋₆-cycloaliphatic residue or the 3 to 7 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(O)—O—C₁₋₄-aliphatic residue, OCF₃, SH, SCF₃, S—C₁₋₄-aliphatic residue, CF₃, CN, C₁₋₄-aliphatic residue and C(═O) OH, on the condition that if R¹¹ denotes a 3 to 7 membered heterocycloaliphatic residue, the 3 to 7 membered heterocycloaliphatic residue is linked via a carbon atom, and R¹² denotes C₁₋₆-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄aliphatic residue, or R¹¹ and R¹² form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue, C(═O)—OH, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the 3 to 10 membered heterocycloaliphatic residue formed by R¹¹ and R¹² together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O)—OH, C(═O)—CH₃, C(═O)C₂H₅, C(═O)OCH₃ and C(═O)—OC₂H₅, C₃₋₆-cycloaliphatic residue, 3 to 7 membered heterocycloaliphatic residue,

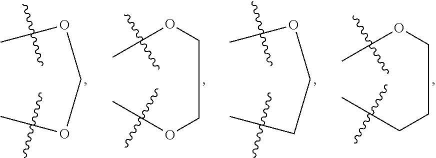 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, R³ denotes C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue, C(═O)—O—C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue, C(═O) OH, C(═O)—O—C₁₋₄-aliphatic residue, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the C₃₋₁₀-cycloaliphatic residue or the 3 to 10 membered heterocycloaliphatic residue may in each case optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, C(═O)—O—C₁₋₄-aliphatic residue, OCF₃, SH, SCF₃, S—C₁₋₄-aliphatic residue, CF₃, CN, C₁₋₄-aliphatic residue and C(═O) OH, on the condition that if R³ denotes a 3 to 10 membered heterocycloaliphatic residue, the 3 to 10 membered heterocycloaliphatic residue is linked via a carbon atom, or denotes aryl or heteroaryl, in each case unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, C₃₋₆-cycloaliphatic residue, 3 to 7 membered

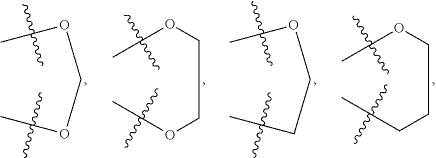 [SMILES: *OCO* ; *OCCO* ; *CCO* ; *CCCO*]

heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), an N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the aryl or the heteroaryl residue may in each case be optionally linked via a C₁₋₄-aliphatic group, which in turn may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN and C(═O) OH, R⁴ denotes H or C₁₋₁₀-aliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, or R³ and R⁴ form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue, C(═O) OH, C₃₋₆-cycloaliphatic residue and 3 to 7 membered heterocycloaliphatic residue, wherein the C₁₋₄-aliphatic residue in each case may be unsubstituted or mono- or polysubstituted with at least one substituent selected from the group consisting of F, Cl, Br, I, ═O, OH, OCF₃, CF₃ and unsubstituted O—C₁₋₄-aliphatic residue, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, ═O, O—C₁₋₄-aliphatic residue, OCF₃, SH, SCF₃, S—C₁₋₄-aliphatic residue, CF₃, CN, C₁₋₄-aliphatic residue and C(═O) OH, and wherein the 3 to 10 membered heterocycloaliphatic residue formed by R³ and R⁴ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, C₃₋₆-cycloaliphatic residue, 3 to 7 membered heterocycloaliphatic residue,

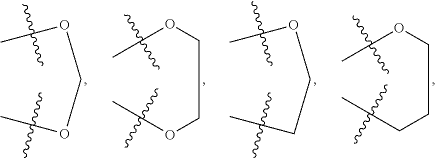 [SMILES: *OCO* ; *OCCO* ; *CCO* ; *CCCO*]

benzyl, phenyl, thienyl, pyridyl, furyl, thiazolyl and oxazolyl, and wherein the 3 to 10 membered heterocycloaliphatic residue formed by R³ and R⁴ together with the nitrogen atom connecting them may optionally be condensed with a C₃₋₁₀-cycloaliphatic residue or a 3 to 10 membered heterocycloaliphatic residue, wherein the C₃₋₁₀-cycloaliphatic residue or the 3 to 10 membered heterocycloaliphatic residue condensed in this way can for their part be respectively unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, ═O, OH, O—C₁₋₄-aliphatic residue, OCF₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, O—C₁₋₄-aliphatic residue, OCF₃, OCH₂CH₂OH, OCH₂OCH₃, SH, SCF₃, S—C₁₋₄-aliphatic residue, CF₃, CN, C₁₋₄-aliphatic residue, C(═O) OH, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, NO₂, NH₂, NH(C₁₋₄-aliphatic residue), N(C₁₋₄-aliphatic residue)₂, OH, —O, O—C₁₋₄-aliphatic residue, OCF₃, SH, SCF₃, S—C₁₋₄-aliphatic residue, CF₃, CN, C₁₋₄-aliphatic residue and C(═O) OH.

In further embodiments, the compound is according to formula 21 wherein n denotes 0 and is according to the formula:

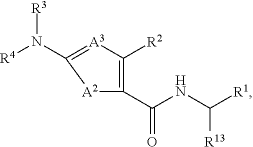

wherein, A² represents O and A³ represents CR⁸; or A² represents S and A³ represents CR⁸; or A² represents NR⁷ and A³ represents CR⁸; or A² represents O and A³ represents N; or A² represents S and A³ represents N; or A² represents NR⁷ and A³ represents N.

In further embodiments, the compound is according to formula 21 wherein n denotes 1 and is according to the formula:

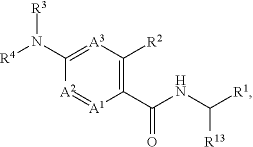

wherein: A¹ represents N and A² represents CR⁶ and A³ represents CR⁸; or A¹ represents CR⁵ and A² represents N and A³ represents CR⁸; or A¹ represents N and A² represents N and A³ represents CR⁸; or A¹ represents N and A² represents CR⁶ and A³ represents N; or A¹ represents CR⁵ and A² represents N and A³ represents N; or A¹ represents N and A² represents N and A³ represents N.

In further embodiments, R⁵ denotes F, Cl, CH₃, OCH₃ or CH₂CH₃; and/or R⁶ denotes H; and/or R⁷ denotes CH₃, CH₂CH₃ or cyclopropyl; and/or R⁸ denotes H.

In further embodiments, R¹ represents the partial structure:

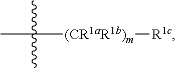

wherein: m denotes 0, 1, or 2, R^{1a} and R^{1b} each independently of one another represent H, F, Cl, Br, I, O—C₁₋₄-aliphatic residue or C₁₋₄-aliphatic residue, R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue, or denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃C₁₋₄-aliphatic residue and C(═O) OH.

In further embodiments, m denotes 1 or 2, R^{1a} and R^{1b} represent H, R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue, or m denotes 0 and R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃C₁₋₄-aliphatic residue and C(═O) OH.

In further embodiments, R² is selected from the group consisting of CH₃, C₂H₅, CH₂CH₂CH₃, CH(CH₃)₂, CH₂CH₂CH₂CH₃, CH₂CH(CH₃)₂, CH(CH₃) CH₂CH₃, C(CH₃)₃, CH₂-cyclopropyl, OCH₃, OC₂H₅, OCH₂CH₂CH₃, OCH(CH₃)₂, O-cyclopropyl, SCH₃, SC₂H₅, SCH₂CH₂CH₃, SCH(CH₃)₂, S-cyclopropyl, cyclopropyl, cyclobutyl, cyclopentyl, cyclohexyl, N(CH₃)₂, N(CH₃) C₂H₅, N(CH₃) CH₂CH₂CH₃, N(CH₃) CH(CH₃)₂, N(CH₃)-cyclopropyl, N(C₂H₅)₂, N(C₂H₅) CH₂CH₂CH₃, N(C₂H₅) CH(CH₃)₂, N(C₂H₅)-cyclopropyl, N-aziridinyl, N-azetidinyl, N-pyrrolidinyl, N-piperidinyl or N-morpholinyl, in each case unsubstituted or mono- or polysubstituted with F, OH and/or OCH₃.

In further embodiments, R³ represents the partial structure:

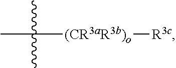

wherein: o denotes 0, 1, 2 or 3, R^{3a} and R^{3b} each independently of one another represent H, F, Cl, Br, I, O—C₁₋₄-aliphatic residue or C₁₋₄-aliphatic residue or together denote ═O, and R^{3c} denotes C₁₋₄-aliphatic residue, unsubstituted or mono- or polysubstituted with at least one substituent selected from the group consisting of F, Cl, Br, I, —O, O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and O—C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue, wherein the C₁₋₄-aliphatic residue in each case may be unsubstituted or mono- or polysubstituted with at least one substituent selected from the group consisting of F, Cl, CF₃ and unsubstituted O—C₁₋₄-aliphatic residue, or denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl, may in each case may be unsubstituted or mono- or polysubstituted, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, CF₃, C₁₋₄-aliphatic residue and C(═O)—OH, and R⁴ denotes H or unsubstituted C₁₋₄-aliphatic residue or C₁₋₄-aliphatic residue, monosubstituted with OCH₃, or R³ and R⁴ form together with the nitrogen atom connecting them a 3 to 10 membered heterocycloaliphatic residue, unsubstituted or mono- or polysubstituted with at least one substituent selected from the group consisting of F, Cl, Br, I, OH, —O, C(═O) OH, O—C₁₋₄-aliphatic residue, OCF₃, SCF₃, S—C₁₋₄-aliphatic residue, CF₃, C₁₋₄-aliphatic residue, cyclopropyl, cyclobutyl and cyclopentyl, wherein the C₁₋₄-aliphatic residue is in each case unsubstituted or mono- or polysubstituted with at least one substituent selected from the group consisting of F, Cl, OH, ═O, CF₃ and O—C₁₋₄-aliphatic residue, and wherein the 3 to 10 membered heterocycloaliphatic residue formed by R³ and R⁴ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, O—C₁₋₄-aliphatic residue, OCF₃, SCF₃, S—C₁₋₄-aliphatic residue, CF₃, C₁₋₄-aliphatic residue, C(═O) OH and C₃₋₆-cycloaliphatic residue, wherein the C₁₋₄-aliphatic residue in each case may be unsubstituted or mono- or polysubstituted with at least one substituent selected from the group consisting of F, Cl, OH, OCF₃, CF₃ and unsubstituted O—C₁₋₄-aliphatic residue, and wherein the C₃₋₆-cycloaliphatic residue may in each case may be unsubstituted or mono- or polysubstituted with at least one substituent selected from the group consisting of F, Cl, Br, I, OH, ═O, O—C₁₋₄-aliphatic residue, OCF₃, SCF₃, S—C₁₋₄-aliphatic residue, CF₃, C₁₋₄-aliphatic residue and C(═O) OH, and wherein the 3 to 10 membered heterocycloaliphatic residue formed by R³ and R⁴ together with the nitrogen atom connecting them may optionally be condensed with a C₃₋₆-cycloaliphatic residue, or a 3 to 7 membered heterocycloaliphatic residue, wherein the C₃₋₆-cycloaliphatic residue or the 3 to 7 membered heterocycloaliphatic residue condensed in this way can for their part be respectively unsubstituted or mono- or polysubstituted with at least one substituent selected from the group consisting of F, Cl, Br, I, —O, OH, O—C₁₋₄-aliphatic residue, OCF₃, SCF₃, CF₃, CN, C₁₋₄-aliphatic residue, C(═O) OH, C(═O)CH₃, C(═O)C₂H₆, C(═O)OCH₃ and C(═O)OC₂H₆.

In further embodiments, R³ represents the partial structure:

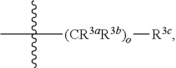

Wherein, o denotes 0, 1, 2 or 3, R^{3a} and R^{3b} each independently of one another represent H, F, CH₃ or OCH₃, or together denote ═O, R^{3c} denotes C₁₋₄-aliphatic residue, unsubstituted or mono- or polysubstituted with at least one substituent selected from the group consisting of F, Cl, Br, I, ═O, O—C₁₋₄-aliphatic residue, and CF₃, or denotes cyclopropyl, cyclobutyl, cyclopentyl or cyclohexyl, pyrrolidinyl, morpholinyl, piperazinyl, piperidinyl or tetrahydropyranyl, in each case unsubstituted or mono- or polysubstituted with at least one substituent selected from the group consisting of F, Cl, Br, I, O—C₁₋₄-aliphatic residue, CF₃ and C₁₋₄-aliphatic residue, or denotes aryl or heteroaryl, in each case unsubstituted or mono- or polysubstituted with at least one substituent selected from the group consisting of F, Cl, Br, OH, O—C₁₋₄-aliphatic residue, OCF₃, CF₃, CN, and C₁₋₄-aliphatic residue, and R⁴ denotes H, CH₃, CH₂CH₃, CH₂CH₂OCH₃ or CH₂CH₂CH₂OCH₃, or R³ and R⁴ form together with the nitrogen atom connecting them a heteroaliphatic residue, selected from the group consisting of morpholinyl, piperidinyl, pyrrolidinyl, azetidinyl, oxazepanyl, tetrahydroquinolinyl, tetrahydroisoquinolinyl, thiomorpholinyl, azepanyl, tetrahydroimidazo[1,2-a]pyrazinyl, octahydropyrrolo[1,2-a]pyrazinyl,

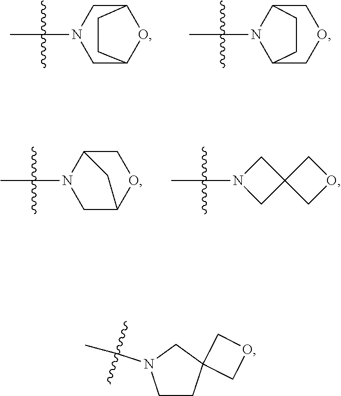 [SMILES: *N1CC2CCC(C1)O2 ; *N1C2CCC1COC2 ; *N1CC2CC1CO2 ; *N1CC2(COC2)C1 ; *N1CCC2(COC2)C1]

dihydroindolinyl, or dihydroisoindolyl, in each case unsubstituted or mono- or polysubstituted with at least one substituent selected from the group consisting of F, Cl, OH, ═O, C(═O) OH, OCH₃, OCH₂CH₃, OCF₃, SCF₃, CF₃, C(═O)CH₃, C(═O)OCH₃, CH₂CF₃, CH₂OH, CH₂OCH₃, CH₂CH₂OCH₃, CH₃, CH₂CH₃, CH₂CH₂CH₃, CH(CH₃)₂ and cyclopropyl.

In further embodiments, A¹ represents CR⁵, N; A² represents CR⁶, N, O, S or NR⁷; A³ represents CR⁸ or N, and n denotes 0 or 1, on the condition, that if n denotes 0, then A² represents O, S or NR⁷, or if n denotes 1, then A² represents CR⁶ or N, wherein R⁵ denotes F, Cl, CH₃, OCH₃ or CH₂CH₃; and/or R⁶ denotes H; and/or R⁷ denotes CH₃, CH₂CH₃ or cyclopropyl; and/or R⁸ denotes H; with the proviso, that, if n denotes 1, then at least one of A¹, A² and A³ denotes N, with the proviso, that if n denotes 1 and A³ denotes N, then A¹ and/or A² denotes N, and with the proviso, that if n denotes 1 and A² denotes N and A¹ denotes CR⁵ and A³ denotes CR⁸, then R⁵ denotes F, Cl, CH₃, CF₃, CHF₂ or CH₂F; R¹³ represents H or CH₃; R¹ represents the partial structure:

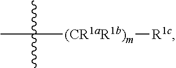

wherein, m denotes 1 or 2, R^{1a} and R^{1b} represent H and R^{1c} denotes C₁₋₄-aliphatic residue, unsubstituted or mono- or polysubstituted with at least one substituent selected from the group consisting of F, Cl, Br, I, O—C₁₋₄-aliphatic residue, CF₃ and C₁₋₄-aliphatic residue, or denotes C₃₋₁₀-cycloaliphatic residue or 3 to 10 membered heterocycloaliphatic residue, in each case unsubstituted or mono- or polysubstituted with at least one substituent selected from the group consisting of F, Cl, Br, I, O—C₁₋₄-aliphatic residue, CF₃ and C₁₋₄-aliphatic residue, or wherein m denotes 0 and R^{1c} denotes aryl or heteroaryl, in each case unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)—CH₃, C(═O)C₂H₅, C(═O)OCH₃, C(═O)OC₂H₅, C₃₋₆-cycloaliphatic residue, 3 to 7 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, O—C₁₋₄-aliphatic residue, OCF₃, CF₃, CN, C₁₋₄-aliphatic residue, C(═O)CH₃, C(O)C₂H₅, C(═O)OCH₃ and C(═O)OC₂H₅, and wherein the C₃₋₆-cycloaliphatic residue and the 3 to 7 membered heterocycloaliphatic residue may in each case may be unsubstituted or mono- or polysubstituted with at least one substituent selected from the group consisting of F, Cl, Br, I, OH, ═O, an O—C₁₋₄-aliphatic residue, OCF₃, CF₃C₁₋₄-aliphatic residue and C(═O) OH; R² is selected from the group consisting of CH₃, C₂H₅, CH₂CH₂CH₃, CH(CH₃)₂, CH₂CH₂CH₂CH₃, CH₂CH(CH₃)₂, CH(CH₃) CH₂CH₃, C(CH₃)₃, CH₂-cyclopropyl, OCH₃, OC₂H₅, OCH₂CH₂CH₃, OCH(CH₃)₂, O-cyclopropyl, SCH₃, SC₂H₅, SCH₂CH₂CH₃, SCH(CH₃)₂, S-cyclopropyl, cyclopropyl, cyclobutyl, cyclopentyl, cyclohexyl, N(CH₃)₂, N(CH₃) C₂H₅, N(CH₃) CH₂CH₂CH₃, N(CH₃) CH(CH₃)₂, N(CH₃)-cyclopropyl, N(C₂H₅)₂, N(C₂H₅) CH₂CH₂CH₃, N(C₂H₅) CH(CH₃)₂, N(C₂H₅)-cyclopropyl, N-aziridinyl, N-azetidinyl, N-pyrrolidinyl, N-piperidinyl or N-morpholinyl, in each case unsubstituted or mono- or polysubstituted with F, OH and/or OCH₃; R³ represents the partial structure:

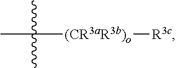

wherein: denotes 0, 1, 2 or 3, R^{3a} and R^{3b} each independently of one another represent H, F, CH₃ or OCH₃, or together denote ═O, R^{3c} denotes C₁₋₄-aliphatic residue, unsubstituted or mono- or polysubstituted with at least one substituent selected from the group consisting of F, Cl, Br, I, ═O, O—C₁₋₄-aliphatic residue, and CF₃, or denotes cyclopropyl, cyclobutyl, cyclopentyl or cyclohexyl, pyrrolidinyl, morpholinyl, piperazinyl, piperidinyl or tetrahydropyranyl, in each case unsubstituted or mono- or polysubstituted with at least one substituent selected from the group consisting of F, Cl, Br, I, O—C₁₋₄-aliphatic residue, CF₃ and C₁₋₄-aliphatic residue, or denotes aryl or heteroaryl, in each case unsubstituted or mono- or polysubstituted with at least one substituent selected from the group consisting of F, Cl, Br, OH, O—C₁₋₄-aliphatic residue, OCF₃, CF₃, CN, and C₁₋₄-aliphatic residue, and R⁴ denotes H, CH₃, CH₂CH₃, CH₂CH₂OCH₃ or CH₂CH₂CH₂OCH₃, or R³ and R⁴ form together with the nitrogen atom connecting them a morpholinyl, piperidinyl, pyrrolidinyl, azetidinyl, oxazepanyl, tetrahydroquinolinyl, tetrahydroisoquinolinyl, thiomorpholinyl, azepanyl, tetrahydroimidazo[1,2-a]pyrazinyl, octahydropyrrolo[1,2-a]-pyrazinyl,

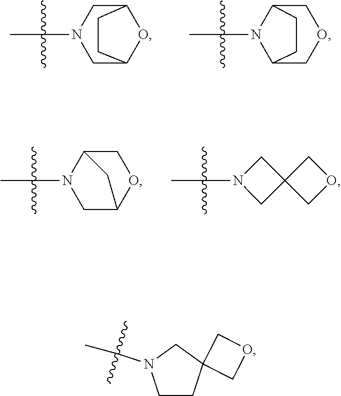 [SMILES: *N1CC2CCC(C1)O2 ; *N1C2CCC1COC2 ; *N1CC2CC1CO2 ; *N1CC2(COC2)C1 ; *N1CCC2(COC2)C1]

dihydroindolinyl, or dihydroisoindolyl, in each case unsubstituted or mono- or polysubstituted with at least one substituent selected from the group consisting of F, Cl, OH, ═O, C(═O) OH, OCH₃, OCH₂CH₃, OCF₃, SCF₃, CF₃, C(═O)CH₃, C(═O)OCH₃, CH₂CF₃, CH₂OH, CH₂OCH₃, CH₂CH₂OCH₃, CH₃, CH₂CH₃, CH₂CH₂CH₃, CH(CH₃)₂ and cyclopropyl.

In further embodiments, the Kv7 channel activator is selected from the group consisting of: N-[(4-Chlorophenyl)-methyl]-4-ethylsulfanyl-6-methyl-2-morpholin-4-yl-pyrimidine-5-carboxylic acid amide; N-[(4-Chlorophenyl)-methyl]-4,6-dimethoxy-2-morpholin-4-yl-pyrimidine-5-carboxylic acid amide; N-[(4-Chlorophenyl)-methyl]-4-ethylsulfanyl-2-methyl-6-morpholin-4-yl-pyridine-3-carboxylic acid amide; N-[(4-Chlorophenyl)-methyl]-2-methyl-6-morpholin-4-yl-4-propyl-pyridine-3-carboxylic acid amide; N-(4,4-Dimethyl-pentyl)-4-ethoxy-2-methyl-6-morpholin-4-yl-pyridine-3-carboxylic acid amide; N-(4,4-Dimethyl-pentyl)-4-[(3R)-3-fluoro-pyrrolidin-1-yl]-2-methyl-6-morpholin-4-yl-pyridine-3-carboxylic acid amide; N-[(4-Chlorophenyl)-methyl]-3-isopropyl-5-morpholin-4-yl-pyrazine-2-carboxylic acid amide; N-[(4-Chlorophenyl)-methyl]-3-isopropyl-5-morpholin-4-yl-pyridine-2-carboxylic acid amide; 4-Isopropyl-2-(methyl-tetrahydro-pyran-4-yl-amino)-N-[1-[3-(trifluoromethyloxy)-phenyl]-ethyl]-thiazole-5-carboxylic acid amide; 4-Ethylsulfanyl-N-[(3-fluorophenyl)-methyl]-6-methyl-2-morpholin-4-yl-pyrimidine-5-carboxylic acid amide; 4-Ethylsulfanyl-N-[(3-fluorophenyl)-methyl]-2-methyl-6-morpholin-4-yl-pyridine-3-carboxylic acid amide; 4-Ethylsulfanyl-N-[(4-fluorophenyl)-methyl]-2-methyl-6-morpholin-4-yl-pyridine-3-carboxylic acid amide; N-(4,4-Dimethyl-pentyl)-4-ethylsulfanyl-2-methyl-6-morpholin-4-yl-pyridine-3-carboxylic acid amide; N-[(4-Chlorophenyl)-methyl]-4-ethylsulfanyl-2-methyl-6-[(3R)-3-methyl-morpholin-4-yl]-pyridine-3-carboxylic acid amide; N-(4,4-Dimethyl-pentyl)-4-ethylsulfanyl-2-methyl-6-[(3R)-3-methyl-morpholin-4-yl]-pyridine-3-carboxylic acid amide; N-[(4-Chlorophenyl)-methyl]-4-cyclopropyl-2-methyl-6-morpholin-4-yl-pyridine-3-carboxylic acid amide; N-[(4-Chlorophenyl)-methyl]-4-isopropyl-2-methyl-6-morpholin-4-yl-pyridine-3-carboxylic acid amide; N-[(4-Chlorophenyl)-methyl]-4-ethoxy-2-methyl-6-morpholin-4-yl-pyridine-3-carboxylic acid amide; N-[(4-Chlorophenyl)-methyl]-4-dimethylamino-2-methyl-6-morpholin-4-yl-pyridine-3-carboxylic acid amide; N-[(4-Chlorophenyl)-methyl]-3-(1-methyl-propyl)-5-morpholin-4-yl-pyrazine-2-carboxylic acid amide; N-(2-Cyclopentyl-ethyl)-3-(1-methyl-propyl)-5-morpholin-4-yl-pyrazine-2-carboxylic acid amide; 4-Isopropyl-2-[methyl-(tetrahydro-pyran-4-yl-methyl)-amino]-N-[1-[3-(trifluoromethyloxy)-phenyl]-ethyl]-thiazole-5-carboxylic acid amide; N-[(4-Chlorophenyl)-methyl]-4-isopropyl-2-(methyl-tetrahydro-pyran-4-yl-amino)-thiazole-5-carboxylic acid amide; N-[(4-Chlorophenyl)-methyl]-4-isopropyl-2-[methyl-(tetrahydro-pyran-4-yl-methyl)-amino]-thiazole-5-carboxylic acid amide; N-[(4-Chlorophenyl)-methyl]-4-isopropyl-2-morpholin-4-yl-thiazole-5-carboxylic acid amide; N-[(4-Chlorophenyl)-methyl]-4-isopropyl-2-piperidin-1-yl-thiazole-5-carboxylic acid amide; N-(4,4-Dimethyl-pentyl)-4-isopropyl-2-piperidin-1-yl-thiazole-5-carboxylic acid amide; N-[(4-Chlorophenyl)-methyl]-4-isopropyl-2-([1,4]oxazepan-4-yl)-thiazole-5-carboxylic acid amide; N-(4,4-Dimethyl-pentyl)-4-isopropyl-2-([1,4]oxazepan-4-yl)-thiazole-5-carboxylic acid amide; N-[(3,4-Difluoro-phenyl)-methyl]-2-morpholin-4-yl-4-propyl-thiazole-5-carboxylic acid amide; N-[(4-Chlorophenyl)-methyl]-4-isopropyl-2-morpholin-4-yl-oxazole-5-carboxylic acid amide; N-(4,4-Dimethyl-pentyl)-4-isopropyl-2-morpholin-4-yl-oxazole-5-carboxylic acid amide; N-(4,4-Dimethyl-pentyl)-5-isopropyl-3-methyl-2-morpholin-4-yl-3H-imidazole-4-carboxylic acid amide; N-[(4-Chlorophenyl)-methyl]-5-isopropyl-3-methyl-2-morpholin-4-yl-3H-imidazole-4-carboxylic acid amide; 2-(Methyl-tetrahydro-pyran-4-yl-amino)-4-(trifluoromethyl)-N-[1-[3-(trifluoromethyloxy)-phenyl]-ethyl]-thiazole-5-carboxylic acid amide; 2-(Methyl-tetrahydro-pyran-4-yl-amino)-4-(trifluoromethyl)-N-[[3-(trifluoromethyloxy)-phenyl]-methyl]-thiazole-5-carboxylic acid amide; N-[1-(4-Chlorophenyl)-ethyl]-2-(methyl-tetrahydro-pyran-4-yl-amino)-4-(trifluoromethyl)-thiazole-5-carboxylic acid amide; N-[(4-Chlorophenyl)-methyl]-2-(methyl-tetrahydro-pyran-4-yl-amino)-4-(trifluoromethyl)-thiazole-5-carboxylic acid amide; N-[(4-Chlorophenyl)-methyl]-2-[methyl-(tetrahydro-pyran-4-yl-methyl)-amino]-4-(trifluoromethyl)-thiazole-5-carboxylic acid amide; 2-Morpholin-4-yl-4-(trifluoromethyl)-N-[[3-(trifluoromethyloxy)-phenyl]-ethyl]-thiazole-5-carboxylic acid amide; 2-Morpholin-4-yl-4-(trifluoromethyl)-N-[1-[3-(trifluoromethyloxy)-phenyl]-ethyl]-thiazole-5-carboxylic acid amide; N-[1-(4-Chlorophenyl)-ethyl]-2-morpholin-4-yl-4-(trifluoromethyl)-thiazole-5-carboxylic acid amide; N-[(4-Chlorophenyl)-methyl]-2-morpholin-4-yl-4-(trifluoromethyl)-thiazole-5-carboxylic acid amide; 2-Morpholin-4-yl-4-(trifluoromethyl)-N-[1-[3-(trifluoromethyl)phenyl]-ethyl]-thiazole-5-carboxylic acid amide; in the form of a free compound, a solvate and/or a physiologically acceptable salt.

### Formula 22

In an embodiment, the Kv7 channel activator may be selected from one of the following compounds according to Formula 22. Such compounds are described in U.S. Pat. No. 9,278,103 issued Mar. 8, 2016 and corresponding to U.S. application Ser. No. 14/230,572 filed Mar. 31, 2014; which is incorporated by reference in its entirety herein. In the case of any conflict of terminology in the context of Formula 22, this reference incorporated by reference herein controls.

In an embodiment, the Kv7 channel activator is a compound according to formula 22:

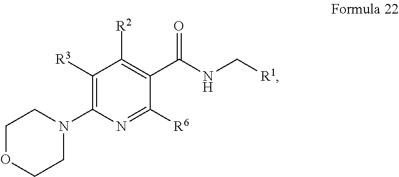

wherein, R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents F; Cl; Br; I; CN; CF₃; C(═O) H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R⁶ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁶ denotes a 3 to 10 membered heterocycloaliphatic residue, the 3 to 10 membered heterocycloaliphatic residue is linked via a carbon atom, or denotes, O—R⁸, wherein R⁸ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁸ denotes a 3 to 10 membered heterocycloaliphatic residue, the 3 to 10 membered heterocycloaliphatic residue is linked via a carbon atom, in which an “aliphatic group” and an “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)-C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)-C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH-C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(—O)-C₁₋₄-aliphatic residue, a C(—O)—O—C₁₋₄-aliphatic residue, a C₃₋₆-cycloaliphatic residue, a 3 to 6 membered heterocycloaliphatic residue, C(O)—NH₂, a C(═O)—NH(C₁₋₄ aliphatic residue), and a C(═O)—N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of

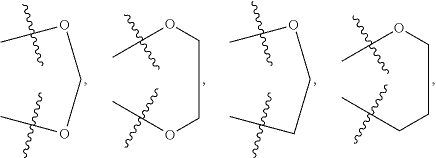 [SMILES: *OCO* ; *OCCO* ; *CCO* ; *CCCO*]

F, Cl, Br, I, NO₂, NH₂, an NH(C₁₋₄ aliphatic residue), an N(C₁₋₄ aliphatic residue)₂, an NH—C(═O)—C₁₋₄ aliphatic residue, an NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂; said compound being in the form of a free compound, the racemate, a mixture of enantiomers, diastereomers, or of enantiomers and diastereomers in any mixing ratio, or of an individual enantiomer or diastereomer, or in the form of a salt of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, a S(═O)₂-C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an 0-C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH) C₁₋₄ aliphatic residue), an N(C₁₋₄ aliphatic residue, OH, an O—C₁₋₄ aliphatic residue, OCF₃, SH, SCF₃, a S—C₁₋₄ aliphatic residue, a S(═O)₂-C₁₋₄-aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic

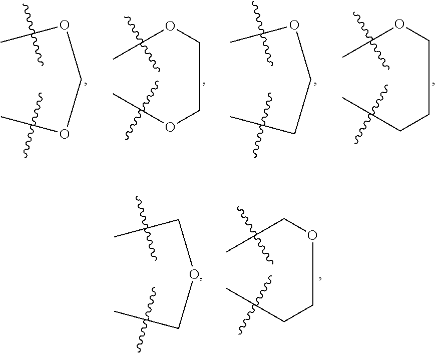 [SMILES: *OCO* ; *OCCO* ; *CCO* ; *CCCO* ; *COC* ; *CCOC*]

residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent sleeted from the group consisting of F,

O—C₁₋₄ aliphatic residue, OCF₃, SH, SCF₃, a S—C₁₋₄ aliphatic residue, CF₃, CN and C(═O) OH, R² represents F; Cl; Br; I; CN; CF₃; NO₂; OCF₃; SCF₃; a C₁₋₄-aliphatic residue, a S—C₁₋₄-aliphatic residue, or a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, —O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue, R³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue, R⁶ denotes a C₂₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, and C(═O) OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁶ denotes a 3 to 10 membered heterocycloaliphatic residue, the binding is carried out via a carbon atom of the 3 to 10 membered heterocycloaliphatic residue, or R⁶ denotes O—R⁸, wherein R⁸ represents a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄ aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or represent a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁸ denotes a 3 to 10 membered heterocycloaliphatic residue, the binding is carried out via a carbon atom of the 3 to 10 membered heterocycloaliphatic residue.

In further embodiments, R¹ represents the partial structure:

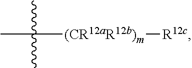

wherein: m denotes 0, 1, 2, 3 or 4, R^{12a} and R^{12b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, and R^{12c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, a S(═O)₂-C₁₋₄ aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, a S(═O)₂-C₁₋₄ aliphatic residue, CF₃, CN, a C₁₋₄ aliphatic residue, C(O)—OH, C(—O)—CH₃, C(═O)—C₂H₅, C(═O)—O—CH₃ and C(═O)—O—C₂H₅, a C₃₋₆ cycloaliphatic residue, a 3 to 6 membered

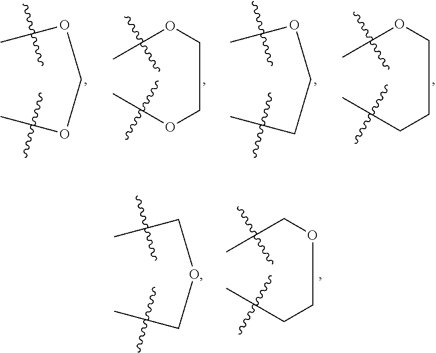 [SMILES: *OCO* ; *OCCO* ; *CCO* ; *CCCO* ; *COC* ; *CCOC*]

heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

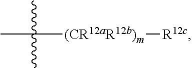

wherein: m denotes 0, 1, or 2 or 3, R^{12a} and R^{12b} each independently of one another represent H, F, Cl, Br, I, OH, an O—C₁₋₄ aliphatic residue or a C₁₋₄ aliphatic residue or together denote ═O, and R^{12c} denotes a C₁₋₄ aliphatic residue, unsubstituted or mono- or polysubstituted with at least one substituent selected from the group consisting of F, Cl, Br, I, OH, an O—C₁₋₄ aliphatic residue, CN, a S(═O)₂-C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄ aliphatic residue in each case may be unsubstituted or mono- or polysubstituted with at least one substituent selected from the group consisting of F, Cl, CF₃ and an unsubstituted O—C₁₋₄-aliphatic residue, or an aryl or heteroaryl, in each case unsubstituted or mono- or polysubstituted with at least one substituent selected from the group consisting of F, Cl, Br, I, OH, an O—C₁₋₄ aliphatic residue, OCF₃, OCF₂H, CF₃, CN, a C₁₋₄-aliphatic residue, CH₂—OH, CH₂—OCH₃, S(═O)₂—CH₃, SCF₃, NO₂, N(C₁₋₄ aliphatic residue)₂,

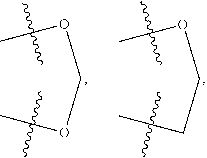 [SMILES: *OCO* ; *CCO*]

C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, —O, an O—C₁₋₄ aliphatic residue, OCF₃, CF₃ a C₁₋₄-aliphatic residue and C(═O)—OH.

In further embodiments, R² represents F; Cl; Br; I; CN; CF₃; NO₂; OCF₃; SCF₃; a C₁₋₄-aliphatic residue, a S—C₁₋₄-aliphatic residue, or a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted O—C₁₋₄-aliphatic residue, cyclopropyl, cyclobutyl, cyclopentyl, cyclohexyl, pyrrolidinyl, piperazinyl, 4-methylpiperazinyl, morpholinyl, or piperidinyl, in each case unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue, and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted O—C₁₋₄-aliphatic residue.

In further embodiments, R³ represents H; F; Cl; Br; I; CN; CF₃; SCF₃; NO₂; OCF₃; a C₁₋₄-aliphatic residue, a O—C₁₋₄-aliphatic residue, or a S—C₁₋₄-aliphatic residue, wherein the C₁₋₄ aliphatic residue may be in each case be unsubstituted or mono- or polysubstituted with at least one substituent selected from the group consisting of F, Cl, Br, I, —O, OH, and an unsubstituted O—C₁₋₄ aliphatic residue.

In further embodiments, R⁶ denotes a C₂₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(—O)—O—C₁₋₄-aliphatic residue, a S—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, OH, —O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue, on the condition that if R⁶ denotes a 3 to 10 membered heterocycloaliphatic residue, the 3 to 10 membered heterocycloaliphatic residue is linked via a carbon atom, or R⁶ denotes O—R⁸ wherein R⁸ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, CF₃, a C(═O)—O—C₁₋₄-aliphatic residue, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, —O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may be bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue, on the condition that if R⁸ denotes a 3 to 10 membered heterocycloaliphatic residue, the 3 to 10 membered heterocycloaliphatic residue is linked via a carbon atom.

In further embodiments, R⁶ denotes a C₂₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S aliphatic residue, CF₃, and a C₁₋₄-aliphatic residue wherein the C₁₋₄-aliphatic residue in each case is unsubstituted, or denotes a C₃₋₁₀-cycloaliphatic residue, or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)-O—C₁₋₄-aliphatic residue, CF₃, and a C₁₋₄-aliphatic residue, wherein the C₁₋₄-aliphatic residue in each case may be unsubstituted or mono- or polysubstituted with OH or an unsubstituted O—C₁₋₄-aliphatic residue, and wherein the C₃₋₁₀-cycloaliphatic residue or the 3 to 10 membered heterocycloaliphatic residue may in each case optionally bridged via a unsubstituted C₁₋₄ aliphatic group, on the condition that if R⁶ denotes a 3 to 10 membered heterocycloaliphatic residue, the 3 to 10 membered heterocycloaliphatic residue is linked via a carbon atom, or R⁶ denotes O—R⁸ wherein R⁸ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄ aliphatic residue, or in each case denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)-O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O C₁₋₄-aliphatic residue, and wherein the C₃₋₁₀-cycloaliphatic residue or the 3 to 10 membered heterocycloaliphatic residue in each case may be bridged via an unsubstituted C₁₋₈ aliphatic group, on the condition that if R⁷ or R⁸ denotes a 3 to 10 membered heterocycloaliphatic residue, the 3 to 10 membered heterocycloaliphatic residue is linked via a carbon atom. In further embodiments, R¹ represents the partial structure:

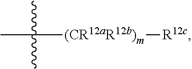

wherein: m is 0, 1 or 2, and R^{12a} and R^{12b} each independently of one another represent H, F, OH, CH₃ or OCH₃, or together denote ═O, R^{12c} denotes a C₁₋₄ aliphatic residue, unsubstituted or mono- or polysubstituted with at least one substituent selected from the group consisting of F, Cl, Br, I, CN, OH, S(═O)₂—CH₃, an unsubstituted O—C₁₋₄ aliphatic residue, and CF₃, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0 or 2, and R^{12a} and R^{12b} each independently of one another represent H, F, OH, CH₃ or OCH₃; and R^{12c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, OCF₂H, CH₂—OH, CH₂—OCH₃, S(═O)₂—CH₃, SCF₃, NO₂, N(CH₃)₂,

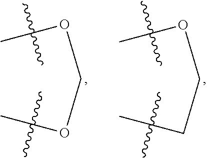 [SMILES: *OCO* ; *CCO*]

CF₃, CN, a C₁₋₄-aliphatic residue, C(═O)—CH₃, C(═O)—C₂H₅, C(═O)—O—CH, C(═O)—O—C₂H₅ and phenyl, wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, R² represents F; Cl; Br; I; CN; CF₃; NO₂; OCF₃; SCF₃; methyl; ethyl; n-propyl; iso-propyl; n-butyl; sec.-butyl; tert.-butyl; CH₂—OH; CH₂—O—CH₃; CH₂—CH₂—OH; CH₂—CH₂ OCH₃; O-methyl; O-ethyl; O—(CH₂)₂—O—CH₃; O—(CH₂)₂—OH; S-Methyl; S-Ethyl; cyclopropyl, cyclobutyl, cyclopentyl, or cyclohexyl, R³ represents H; F; Cl; Br; I; CN; CF₃; SCF₃; NO₂; OCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; or S-Ethyl, R⁶ denotes ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), CH₂—OCH₃, C₂H₄—OCH₃, C₃H₆—OCH₃, cyclopropyl, cyclobutyl, or tetrahydropyranyl, ethenyl or propenyl (—CH₂CH—CH₂, —CH—CH—CH₃, —C((═CH₂)—CH₃), in each case unsubstituted, or R⁶ denotes O—R⁸ wherein R⁸ denotes methyl, ethyl, n-propyl, 2-propyl, n-butyl, isobutyl, sec.-butyl, tert.-butyl, n-pentyl, isopentyl, neopentyl, or n-hexyl, in each case unsubstituted or mono- or polysubstituted with at least one substituent selected from the group consisting of F, Cl, OH, N(C₁₋₄ aliphatic residue) and an O—C₁₋₄-aliphatic residue, or in each case denote CH₂-cyclopropyl or oxetanyl, wherein the C₁₋₄-aliphatic residue in each case is unsubstituted.

In further embodiments, the Kv7 channel activator is selected from the group consisting of: N-[(3,5-Difluoro-phenyl)-methyl]-2-methoxy-4-methyl-6-morpholin-4-yl-pyridine-3-carboxylic acid amide; 9 N-[(3-Fluorophenyl)-methyl]-4-methyl-6-morpholin-4-yl-2-[(E)-prop-1-enyl]-pyridine-3-carboxylic acid amide; 10 N-[(3-Fluorophenyl)-methyl]-4-methyl-6-morpholin-4-yl-2-propyl-pyridine-3-carboxylic acid amide; 19 N-[(3-Fluorophenyl)-methyl]-2-methoxy-4-methyl-6-morpholin-4-yl-pyridine-3-carboxylic acid amide; N-[3-Fluorophenyl)-methyl]-2-(2-methoxy-ethoxy)-4-methyl-6-morpholin-4-yl-pyridine-3-carboxylic acid amide; 2-Ethyl-N-[(3-fluorophenyl)-methyl]-4-methyl-6-morpholin-4-yl-pyridine-3-carboxylic acid amide; N-[(3-Fluorophenyl)-methyl]-2-isopropyl-4-methyl-6-morpholin-4-yl-pyridine-3-carboxylic acid amide; N-[(4-Fluorophenyl)-methyl]-4-methyl-6-morpholin-4-yl-2-[(E)-prop-1-enyl]-pyridine-3-carboxylic acid amide; N-[(4-Fluorophenyl)-methyl]-4-methyl-6-morpholin-4-yl-2-propyl-pyridine-3-carboxylic acid amide 2-Ethoxy-N-[(3-fluorophenyl)-methyl]-4-methyl-6-morpholin-4-yl-pyridine-3-carboxylic acid amide; N-[(4-Fluorophenyl)-methyl]-2-methoxy-4-methyl-6-morpholin-4-yl-pyridine-3-carboxylic acid amide; 2-Ethoxy-N-[(4-fluorophenyl)-methyl]-4-methyl-6-morpholin-4-yl-pyridine-3-carboxylic acid amide; N-(4,4-Dimethyl-pentyl)-2-methoxy-4-methyl-6-morpholin-4-yl-pyridine-3-carboxylic acid amide; N-[(3,4-Difluoro-phenyl)-methyl]-2-methoxy-4-methyl-6-morpholin-4-yl-pyridine-3-carboxylic acid amide; 2-Methoxy-4-methyl-6-morpholin-4-yl-N-[(2-phenyl-phenyl)-methyl]-pyridine-3-carboxylic acid amide; N-(4,4-Dimethyl-pentyl)-2-ethoxy-4-methyl-6-morpholin-4-yl-pyridine-3-carboxylic acid amide; N-[(3,5-Difluoro-phenyl)-methyl]-2-ethoxy-4-methyl-6-morpholin-4-yl-pyridine-3-carboxylic acid amide; N-[(3,4-Difluoro-phenyl)-methyl]-2-ethoxy-4-methyl-6-morpholin-4-yl-pyridine-3-carboxylic acid amide; 2-Ethoxy-4-methyl-6-morpholin-4-yl-N-[(2-phenyl-phenyl)-methyl]-pyridine-3-carboxylic acid amide; 2-Butoxy-N-[(3-fluorophenyl)-methyl]-4-methyl-6-morpholin-4-yl-pyridine-3-carboxylic acid amide; N-[(3-Fluorophenyl)-methyl]-4-methyl-6-morpholin-4-yl-2-propoxy-pyridine-3-carboxylic acid amide; 2-Methoxy-4-methyl-6-morpholin-4-yl-N-[[4-(trifluoromethyl)-phenyl]-methyl]-pyridine-3-carboxylic acid amide; 2-Ethoxy-4-methyl-6-morpholin-4-yl-N-[[4-(trifluoromethyl)-phenyl]-methyl]-pyridine-3-carboxylic acid amide; 2-Ethoxy-4-methyl-6-morpholin-4-yl-N-(4,4,4-trifluoro-butyl)-pyridine-3-carboxylic acid amide; 2-Methoxy-4-methyl-6-morpholin-4-yl-N-(4,4,4-trifluoro-butyl)-pyridine-3-carboxylic acid amide; N-[(4-Chlorophenyl)-methyl]-2-methoxy-4-methyl-6-morpholin-4-yl-pyridine-3-carboxylic acid amide; N-[(4-Chlorophenyl)-methyl]-2-ethoxy-4-methyl-6-morpholin-4-yl-pyridine-3-carboxylic acid amide; N-[(4-Fluorophenyl)-methyl]-2-isopropyl-4-methyl-6-[(3R)-3-methyl-morpholin-4-yl]-pyridine-3-carboxylic acid amide; N-[(4-Fluorophenyl)-methyl]-2-methoxy-4-methyl-6-[(3R)-3-methyl-morpholin-4-yl]-pyridine-3-carboxylic acid amide; 2-Ethoxy-N-[(4-fluorophenyl)-methyl]-4-methyl-6-[(3R)-3-methyl-morpholin-4-yl]-pyridine-3-carboxylic acid amide; N-(4,4-Dimethyl-pentyl)-2-isopropyl-4-methyl-6-[(3R)-3-methyl-morpholin-4-yl]-pyridine-3-carboxylic acid amide; N-(4,4-Dimethyl-pentyl)-2-methoxy-4-methyl-6-[(3R)-3-methyl-morpholin-4-yl]-pyridine-3-carboxylic acid amide; N-(4,4-Dimethyl-pentyl)-2-ethoxy-4-methyl-6-[(3R)-3-methyl-morpholin-4-yl]-pyridine-3-carboxylic acid amide; N-[(4-Fluorophenyl)-methyl]-4-methyl-6-[(3R)-3-methyl-morpholin-4-yl]-2-(1-methyl-propyl)-pyridine-3-carboxylic acid amide; N-(4,4-Dimethyl-pentyl)-4-methyl-6-[(3R)-3-methyl-morpholin-4-yl]-2-(1-methyl-propyl)-pyridine-3-carboxylic acid amide; 2-Cyclopropyl-N-[(4-fluorophenyl)-methyl]-4-methyl-6-[(3R)-3-methyl-morpholin-4-yl]-pyridine-3-carboxylic acid amide; N-[(4-Fluorophenyl)-methyl]-4-methyl-6-[(3R)-3-methyl-morpholin-4-yl]-2-propyl-pyridine-3-carboxylic acid amide; 2-Cyclopropyl-N-(4,4-dimethyl-pentyl)-4-methyl-6-[(3R)-3-methyl-morpholin-4-yl]-pyridine-3-carboxylic acid amide; N-(4,4-Dimethyl-pentyl)-4-methyl-6-[(3R)-3-methyl-morpholin-4-yl]-2-propyl-pyridine-3-carboxylic acid amide; N-[(3-Fluorophenyl)-methyl]-2-methoxy-4-methyl-6-[(3R)-3-methyl-morpholin-4-yl]-pyridine-3-carboxylic acid amide; N-[(4-Chlorophenyl)-methyl]-2-methoxy-4-methyl-6-[(3R)-3-methyl-morpholin-4-yl]-pyridine-3-carboxylic acid amide; N-[(3-Fluorophenyl)-methyl]-4-methyl-2-(1-methyl-propyl)-6-morpholin-4-yl-pyridine-3-carboxylic acid amide; 2-tert-Butyl-N-(4,4-dimethyl-pentyl)-4-methyl-6-morpholin-4-yl-pyridine-3-carboxylic acid amide; N-(4,4-Dimethyl-pentyl)-4-methyl-2-(1-methyl-propyl)-6-morpholin-4-yl-pyridine-3-carboxylic acid amide; N-[(3,4-Difluoro-phenyl)-methyl]-2-methoxy-4-methyl-6-[(3R)-3-methyl-morpholin-4-yl]-pyridine-3-carboxylic acid amide; N-[(3,5-Difluoro-phenyl)-methyl]-2-methoxy-4-methyl-6-[(3R)-3-methyl-morpholin-4-yl]-pyridine-3-carboxylic acid amide; 2-Methoxy-4-methyl-6-[(3R)-3-methyl-morpholin-4-yl]-N-(4,4,4-trifluoro-butyl)-pyridine-3-carboxylic acid amide; 2-Methoxy-4-methyl-6-[(3R)-3-methyl-morpholin-4-yl]-N-[[4-(trifluoromethyl)-phenyl]-methyl]-pyridine-3-carboxylic acid amide; 2-tert-Butyl-N-[(3-fluorophenyl)-methyl]-4-methyl-6-morpholin-4-yl-pyridine-3-carboxylic acid amide; N-[(4-Chlorophenyl)-methyl]-2-isopropyl-4-methyl-6-[(3R)-3-methyl-morpholin-4-yl]-pyridine-3-carboxylic acid amide; N-[(4-Chlorophenyl)-methyl]-4-methyl-6-[(3R)-3-methyl-morpholin-4-yl]-2-propyl-pyridine-3-carboxylic acid amide; N-[(4-Chlorophenyl)-methyl]-2-(2-fluoro-ethoxy)-4-methyl-6-morpholin-4-yl-pyridine-3-carboxylic acid amide; N-[(4-Chlorophenyl)-methyl]-2-(2,2-difluoro-ethoxy)-4-methyl-6-morpholin-4-yl-pyridine-3-carboxylic acid amide; N-[(4-Chlorophenyl)-methyl]-2-(cyclopropyl-methoxy)-4-methyl-6-morpholin-4-yl-pyridine-3-carboxylic acid amide; 2-(2,2-Difluoro-ethoxy)-N-[(4-fluorophenyl)-methyl]-4-methyl-6-morpholin-4-yl-pyridine-3-carboxylic acid amide; N-[(4-Chlorophenyl)-methyl]-2-ethoxy-4-methyl-6-[(3R)-3-methyl-morpholin-4-yl]-pyridine-3-carboxylic acid amide; 2-(Cyclopropyl-methoxy)-N-[(4-fluorophenyl)-methyl]-4-methyl-6-morpholin-4-yl-pyridine-3-carboxylic acid amide; N-[(4-Chlorophenyl)-methyl]-2-isopropyl-6-[(3S)-3-(methoxymethyl)-morpholin-4-yl]-4-methyl-pyridine-3-carboxylic acid amide; N-(4,4-Dimethyl-pentyl)-4-methyl-2-(2-methyl-butyl)-6-morpholin-4-yl-pyridine-3-carboxylic acid amide; N-(4,4-Dimethyl-pentyl)-2-(1,1-dimethyl-propyl)-4-methyl-6-morpholin-4-yl-pyridine-3-carboxylic acid amide; N-[(4-Chlorophenyl)-methyl]-2-cyclopropyl-4-methyl-6-[(3R)-3-methyl-morpholin-4-yl]-pyridine-3-carboxylic acid amide; N-[(4-Chlorophenyl)-methyl]-2-(2-dimethylaminoethyloxy)-4-methyl-6-morpholin-4-yl-pyridine-3-carboxylic acid amide; N-[(4-Chlorophenyl)-methyl]-2-isopropyl-6-[(2S)-2-(methoxymethyl)-morpholin-4-yl]-4-methyl-pyridine-3-carboxylic acid amide; N-[(4-Chlorophenyl)-methyl]-2-cyclopropyl-6-[(2S)-2-(methoxymethyl)-morpholin-4-yl]-4-methyl-pyridine-3-carboxylic acid amide; N-[(4-Fluorophenyl)-methyl]-2-isopropyl-4-methyl-6-morpholin-4-yl-pyridine-3-carboxylic acid amide; N-[(3,4-Difluoro-phenyl)-methyl]-2-isopropyl-4-methyl-6-morpholin-4-yl-pyridine-3-carboxylic acid amide; 2-Isopropyl-4-methyl-6-morpholin-4-yl-N-[[4-(trifluoromethyl)-phenyl]-methyl]-pyridine-3-carboxylic acid amide; N-(4,4-Dimethyl-pentyl)-4-methyl-6-morpholin-4-yl-2-propyl-pyridine-3-carboxylic acid amide; N-(4,4-Dimethyl-pentyl)-2-isopropyl-4-methyl-6-morpholin-4-yl-pyridine-3-carboxylic acid amide; 2-Isopropyl-4-methyl-6-morpholin-4-yl-N-(4,4,4-trifluoro-butyl)-pyridine-3-carboxylic acid amide; N-[(3,5-Difluoro-phenyl)-methyl]-2-isopropyl-4-methyl-6-morpholin-4-yl-pyridine-3-carboxylic acid amide; N-[(3-Fluorophenyl)-methyl]-4-methyl-6-morpholin-4-yl-2-(oxetan-3-yloxy)-pyridine-3-carboxylic acid amide; 4-Methyl-6-morpholin-4-yl-2-propyl-N-(4,4,4-trifluoro-butyl)-pyridine-3-carboxylic acid amide; N-[(3,4-Difluoro-phenyl)-methyl]-4-methyl-6-morpholin-4-yl-2-propyl-pyridine-3-carboxylic acid amide; N-[(3,5-Difluoro-phenyl)-methyl]-4-methyl-6-morpholin-4-yl-2-propyl-pyridine-3-carboxylic acid amide; 4-Methyl-6-morpholin-4-yl-2-propyl-N-[[4-(trifluoromethyl)-phenyl]-methyl]-pyridine-3-carboxylic acid amide; N-[(4-Chlorophenyl)-methyl]-4-methyl-6-morpholin-4-yl-2-propyl-pyridine-3-carboxylic acid amide; N-[(4-Chlorophenyl)-methyl]-2-isopropyl-4-methyl-6-morpholin-4-yl-pyridine-3-carboxylic acid amide; 2-Cyclopropyl-N-(4,4-dimethyl-pentyl)-4-methyl-6-morpholin-4-yl-pyridine-3-carboxylic acid amide; 2-Cyclopropyl-4-methyl-6-morpholin-4-yl-N-(4,4,4-trifluoro-butyl)-pyridine-3-carboxylic acid amide; 2-Cyclopropyl-N-[(3-fluorophenyl)-methyl]-4-methyl-6-morpholin-4-yl-pyridine-3-carboxylic acid amide; 2-Cyclopropyl-N-[(4-fluorophenyl)-methyl]-4-methyl-6-morpholin-4-yl-pyridine-3-carboxylic acid amide; 2-Cyclopropyl-N-[(3,4-difluoro-phenyl)-methyl]-4-methyl-6-morpholin-4-yl-pyridine-3-carboxylic acid amide; 2-Cyclopropyl-N-[(3,5-difluoro-phenyl)-methyl]-4-methyl-6-morpholin-4-yl-pyridine-3-carboxylic acid amide; 2-Cyclopropyl-4-methyl-6-morpholin-4-yl-N-[[4-(trifluoromethyl)-phenyl]-methyl]-pyridine-3-carboxylic acid amide; N-[(4-Chlorophenyl)-methyl]-2-cyclopropyl-4-methyl-6-morpholin-4-yl-pyridine-3-carboxylic acid amide; N-[(4-Chlorophenyl)-methyl]-4-methyl-2-(1-methyl-propyl)-6-morpholin-4-yl-pyridine-3-carboxylic acid amide; N-(4,4-Dimethyl-pentyl)-2-(2-methoxy-ethoxy)-4-methyl-6-morpholin-4-yl-pyridine-3-carboxylic acid amide; 2-Cyclopropyl-N-[[4-fluoro-3-(methoxymethyl)-phenyl]-methyl]-4-methyl-6-morpholin-4-yl-pyridine-3-carboxylic acid amide; N-(4,4-Dimethyl-pentyl)-2-(3-methoxy-propyl)-4-methyl-6-morpholin-4-yl-pyridine-3-carboxylic acid amide; 2-Cyclopropyl-N-[[3-fluoro-4-(methoxymethyl)-phenyl]-methyl]-4-methyl-6-morpholin-4-yl-pyridine-3-carboxylic acid amide; N-[(3-Fluorophenyl)-methyl]-2-(methoxymethyl)-4-methyl-6-morpholin-4-yl-pyridine-3-carboxylic acid amide; N-[(4-Chlorophenyl)-methyl]-2,4-diisopropyl-6-morpholin-4-yl-pyridine-3-carboxylic acid amide; N-(4,4-Dimethyl-pentyl)-2-(2-methoxy-ethyl)-4-methyl-6-morpholin-4-yl-pyridine-3-carboxylic acid amide; N-[(4-Chlorophenyl)-methyl]-2,4-diethyl-6-morpholin-4-yl-pyridine-3-carboxylic acid amide; and N-(4,4-Dimethyl-pentyl)-4-methyl-6-morpholin-4-yl-2-tetrahydro-pyran-4-yl-pyridine-3-carboxylic acid amide, respectively in the form of free compounds; racemic mixtures; mixtures of the enantiomers, diastereomers, or enantiomers and diastereomers in any mixing ratio, or an individual enantiomer or diastereomer; or in the form of the salts of physiologically acceptable acids or bases.

### Formula 23

In an embodiment, the Kv7 channel activator may be selected from one of the following compounds according to Formula 23. Such compounds are described in U.S. Pat. No. 9,168,259 issued Mar. 8, 2016 and corresponding to U.S. application Ser. No. 13/449,472 filed Apr. 18, 2012; U.S. Pat. No. 8,552,200 issued Dec. 12, 2013 and corresponding to U.S. application Ser. No. 13/276,464 filed Oct. 19, 2011; International Publication No. WO2012052167A1, published Apr. 26, 2012 and corresponding to International Application No. PCT/EP2011/005265 filed Apr. 26, 2012; which are incorporated by reference in their entirety herein. In the case of any conflict of terminology in the context of Formula 23, these references incorporated by reference herein control.

In an embodiment, the Kv7 channel activator is a compound according to formula 23:

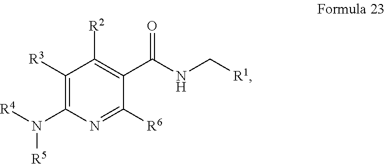

wherein, R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents F; Cl; Br; I; CN; CF₃; C(═O) H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R⁴ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; on the condition that if R⁴ denotes a 3 to 10 membered heterocycloaliphatic residue or a heteroaryl, the 3 to 10 membered heterocycloaliphatic residue or the heteroaryl is linked via a carbon atom; R⁵ denotes H or a C₁₋₁₀-aliphatic residue, unsubstituted or mono- or polysubstituted; or R⁴ and R⁵ form together with the nitrogen atom connecting them a 3 to 10 membered heterocycloaliphatic residue, unsubstituted or mono- or polysubstituted; R⁶ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁶ denotes a 3 to 10 membered heterocycloaliphatic residue, the 3 to 10 membered heterocycloaliphatic residue is linked via a carbon atom, or denotes S—R⁷, O—R⁸ or N(R⁹R¹⁰), wherein R⁷ and R⁸ in each case represent a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷ or R⁸ denotes a 3 to 10 membered heterocycloaliphatic residue, the 3 to 10 membered heterocycloaliphatic residue is linked via a carbon atom, R⁹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if Ro denotes a 3 to 10 membered heterocycloaliphatic residue, the 3 to 10 membered heterocycloaliphatic residue is linked via a carbon atom; R¹⁰ denotes a C₁₋₁₀-aliphatic residue, unsubstituted or mono- or polysubstituted; or R⁹ and R¹⁰ form together with the nitrogen atom connecting them a 3 to 10 membered heterocycloaliphatic residue, unsubstituted or mono- or polysubstituted; in which an “aliphatic group” and an “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, —O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)-C₁₋₄-aliphatic residue, a C(—O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(—O)-N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)-C₁₋₄ aliphatic residue, a NH—S(═O)₂-C₁₋₄ aliphatic residue, —O, OH, OCF₃, a O—C₁₋₄-aliphatic residue, a O—C(═O)-C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)-C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

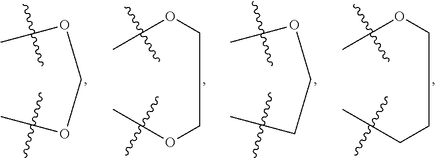 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂-O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂; said compound being in the form of a free compound, the racemate, a mixture of enantiomers, diastereomers, or of enantiomers and diastereomers in any mixing ratio, or of an individual enantiomer or diastereomer, or in the form of a salt of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, a S(═O)₂-C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an

NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, a S(═O)₂-C₁₋₄-aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic

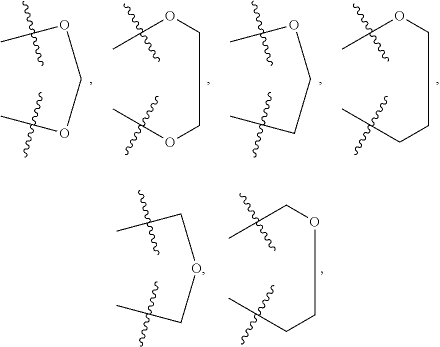 [SMILES: *OCO* ; *OCCO* ; *CCO* ; *CCCO* ; *COC* ; *CCOC*]

residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, preferably a C₁₋₄ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O) OH, R² represents F; Cl; Br; I; CN; CF₃; NO₂; OCF₃; SCF₃; a C₁₋₄-aliphatic residue, a S—C₁₋₄-aliphatic residue, or a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and an O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue, R³ represents H; F; Cl; Br; I; CN; CF₃; SCF₃; NO₂; OCF₃; a C₁₋₄-aliphatic residue, a O—C₁₋₄-aliphatic residue, a S—C₁₋₄-aliphatic residue, wherein the C₁₋₄ aliphatic residue may be in each case be unsubstituted or mono- or polysubstituted with at least one substituent selected from the group consisting of F, Cl, Br, I, ═O, OH, and an unsubstituted O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue, R⁴ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCF₃, SH, SCF₃, a S—C₁₋₄-aliphatic residue, CF₃, CN, a C₁₋₄-aliphatic residue, a C(—O)—O—C₁₋₄-aliphatic residue, and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH, on the condition that if R⁴ denotes a 3 to 10 membered heterocycloaliphatic residue, the 3 to 10 membered heterocycloaliphatic residue is linked via a carbon atom, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

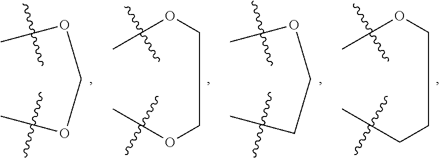 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH, R⁵ denotes H or a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or R⁴ and R⁵ form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(—O)-OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, ═O, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the 3 to 10 membered heterocycloaliphatic residue formed by R⁴ and R⁵ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic

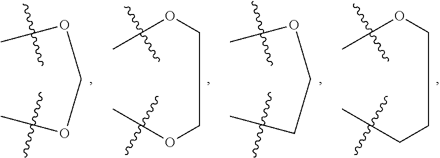 [SMILES: *OCO* ; *OCCO* ; *CCO* ; *CCCO*]

residue, benzyl, phenyl, thienyl, pyridyl, furyl, thiazolyl and oxazolyl, and wherein the 3 to 10 membered heterocycloaliphatic residue formed by R⁴ and R⁵ together with the nitrogen atom connecting them may optionally be condensed with a C₃₋₁₀ cycloaliphatic residue or a 3 to 10 membered heterocycloaliphatic residue, wherein the C₃₋₁₀ cycloaliphatic residue or a 3 to 10 membered heterocycloaliphatic residue condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, ═O, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH, R⁶ denotes a C₂₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁶ denotes a 3 to 10 membered heterocycloaliphatic residue, the binding is carried out via a carbon atom of the 3 to 10 membered heterocycloaliphatic residue, or R⁶ denotes S—R⁷, O—R⁸ or N(R⁹R¹⁰), wherein R⁷ and R⁸ in each case represent a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or in each case represent a C₃₋₁₀-cycloaliphatic residue or a 3 to 10 membered heterocycloaliphatic residue, in each case unsubstituted or mono- or polysubstituted with at least one substituent selected from the group consisting of F,

O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁷ or R⁹ denotes a 3 to 10 membered heterocycloaliphatic residue, the binding is carried out via a carbon atom of the 3 to 10 membered heterocycloaliphatic residue, R⁹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue, and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C(═O)—O—C₁₋₄-aliphatic residue a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, a C(═O)—O—C₁₋₄-aliphatic residue, OCF₃, SH, SCF₃, a S—C₁₋₄ aliphatic residue, CF₃, CN, a C₁₋₄-aliphatic residue and C(═O)—OH, on the condition that if Ro denotes a 3 to 10 membered heterocycloaliphatic residue, the binding is carried out via a carbon atom of the 3 to 10 membered heterocycloaliphatic residue, R¹⁰ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or R⁹ and R¹⁰ form together with the nitrogen atom connecting them a 3 to 10 membered heterocycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁-4 aliphatic residue, CF₃, CN, a C₁₋₄-aliphatic residue and C(═O)—OH, and wherein the 3 to 10 membered heterocycloaliphatic residue formed by R⁹ and R¹⁰ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)O—C₂H₅, a C₃₋₆ cycloaliphatic residue, a 3 to 6 membered heterocycloaliphatic

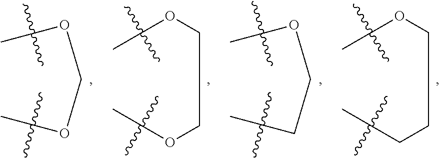 [SMILES: *OCO* ; *OCCO* ; *CCO* ; *CCCO*]

residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

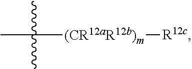

wherein: m denotes 0, 1, 2, 3 or 4, R^{12a} and R^{12b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(—O)-OH, or together denote-O, and R^{12c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, a S(═O)₂-C₁₋₄ aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(—O)-OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, a S(═O)₂-C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered

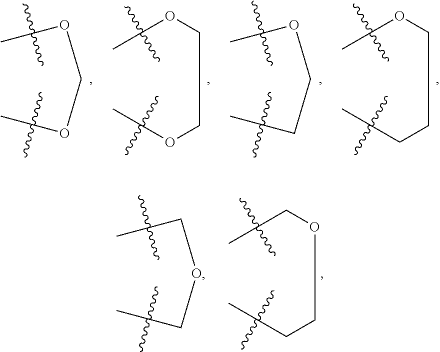 [SMILES: *OCO* ; *OCCO* ; *CCO* ; *CCCO* ; *COC* ; *CCOC*]

heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

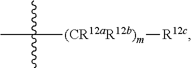

wherein: m denotes 0, 1, or 2 or 3, R^{12a} and R^{12b} each independently of one another represent H, F, Cl, Br, I, OH, an O—C₁₋₄ aliphatic residue or a C₁₋₄ aliphatic residue or together denote ═O, and R^{12c} denotes a C₁₋₄ aliphatic residue, unsubstituted or mono- or polysubstituted with at least one substituent selected from the group consisting of F, Cl, Br, I, OH, an O—C₁₋₄ aliphatic residue, CN, a S(═O)₂-C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or an aryl or heteroaryl, in each case unsubstituted or mono- or polysubstituted with at least one substituent selected from the group consisting of F, Cl, Br, I, OH, an O—C₁₋₄ aliphatic residue, OCF₃, OCF₂H, CF₃, CN, a C₁₋₄-aliphatic residue, CH₂—OH, CH₂—OCH₃, S(═O)₂—CH₃, SCF₃, NO₂, N(C₁₋₄ aliphatic residue)₂,

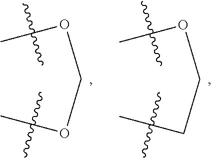 [SMILES: *OCO* ; *CCO*]

C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In further embodiments, R² represents F; Cl; Br; I; CN; CF₃; NO₂; OCF₃; SCF₃; a C₁₋₄-aliphatic residue, a S—C₁₋₄-aliphatic residue, or a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted aliphatic residue, cyclopropyl, cyclobutyl, cyclopentyl, cyclohexyl, pyrrolidinyl, piperazinyl, 4-methylpiperazinyl, morpholinyl, or piperidinyl, in each case unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue, and wherein cyclopropyl, cyclobutyl, cyclopentyl, cyclohexyl, pyrrolidinyl, piperazinyl, 4-methylpiperazinyl, morpholinyl or piperidinyl may in each case be optionally bridged via an C₁₋₄ aliphatic group, which in turn may be unsubstituted or mono- or polysubstituted with at least one substituent selected from the group consisting of F, Cl, OH, an unsubstituted C₁₋₄-aliphatic residue and an unsubstituted aliphatic residue.

In further embodiments, R³ represents H; F; Cl; Br; I; CN; CF₃; SCF₃; NO₂; OCF₃; a C₁₋₄-aliphatic residue, a O—C₁₋₄-aliphatic residue, or a S—C₁₋₄-aliphatic residue, wherein the C₁₋₄ aliphatic residue may be in each case be unsubstituted or mono- or polysubstituted with at least one substituent selected from the group consisting of F, Cl, Br, I, ═O, OH, and an unsubstituted O—C₁₋₄-aliphatic residue.

In further embodiments, R⁴ represents the partial structure:

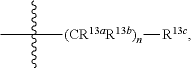

wherein: n denotes 0, 1, 2, or 3, R^{13a} and R^{13b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, and R^{13c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

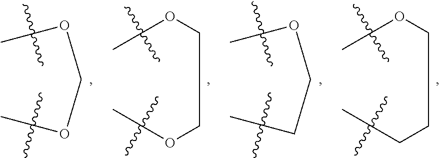 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, R⁵ denotes H or a C₁₋₁₀-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or R⁴ and R⁵ form together with the nitrogen atom connecting them a 3 to 10 membered heterocycloaliphatic residue, unsubstituted or mono- or polysubstituted with at least one substituent selected from the group consisting of F, Cl, Br, I, NO₂, OH, —O, C(═O)—OH, an O—C₁₋₄ aliphatic residue, OCF₃, SH, SCF₃, a S—C₁₋₄ aliphatic residue, CF₃, CN, and a C₁₋₄-aliphatic residue, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, ═O, CF₃ and an unsubstituted O—C₁₋₄-aliphatic residue, wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the 3 to 10 membered heterocycloaliphatic residue formed by R⁴ and R⁵ together with the nitrogen atom connecting them may optionally be condensed with aryl or heteroaryl, wherein the aryl or heteroaryl residues condensed in this way can for their part be respectively unsubstituted or mono- or polysubstituted with at least one substituent selected from the group consisting of F, Cl, Br, I, NO₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, a 3 to 6

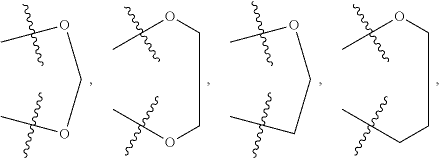 [SMILES: *OCO* ; *OCCO* ; *CCO* ; *CCCO*]

membered heterocycloaliphatic residue, benzyl, phenyl, thienyl, and pyridyl, and wherein the 3 to 10 membered heterocycloaliphatic residue formed by R⁴ and R⁵ together with the nitrogen atom connecting them may optionally be condensed with a C₃₋₁₀ cycloaliphatic residue, preferably cyclopropyl, cyclobutyl or cycclopentyl, or a 3 to 10 membered heterocycloaliphatic residue, preferably oxetanyl or oxiranyl, wherein the C₃₋₁₀ cycloaliphatic residue or the 3 to 10 membered heterocycloaliphatic residue condensed in this way can for their part be respectively unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, —O, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, and pyridyl, may in each case may be unsubstituted or mono- or polysubstituted with at least one substituent selected from the group consisting of F, Cl, Br, I, NO₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, and C(═O)—OH, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH. In further embodiments, R⁴ represents the partial structure:

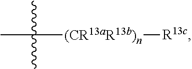

wherein: n denotes 0, 1, 2 or 3, R^{13a} and R^{13b} each independently of one another represent H, F, Cl, Br, I, an O—C₁₋₄ aliphatic residue or a C₁₋₄ aliphatic residue or together denote-O, and R^{13c} denotes a C₁₋₄ aliphatic residue, unsubstituted or mono- or polysubstituted with at least one substituent selected from the group consisting of F, Cl, Br, I, ═O, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH, R⁵ denotes H or an unsubstituted C₁₋₄-aliphatic residue or a C₁₋₄-aliphatic residue monosubstituted with O-methyl, or R⁴ and R⁵ form together with the nitrogen atom connecting them a 3 to 10 membered heterocycloaliphatic residue, unsubstituted or mono- or polysubstituted with at least one substituent selected from the group consisting of F, Cl, Br, I, OH, ═O, C(═O)—OH, an O—C₁₋₄ aliphatic residue, OCF₃, SCF₃, a S—C₁₋₄ aliphatic residue, CF₃, and a C₁₋₄-aliphatic residue, cyclopropyl, cyclobutyl and cyclopentyl, wherein the C₁₋₄-aliphatic residue is in each case unsubstituted or mono- or polysubstituted with at least one substituent selected from the group consisting of F, Cl, OH, ═O, CF₃ and an unsubstituted O—C₁₋₄-aliphatic residue, and wherein the 3 to 10 membered heterocycloaliphatic residue formed by R⁴ and R⁵ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, an O—C₁₋₄ aliphatic residue, OCF₃, SCF₃, a S—C₁₋₄ aliphatic residue, CF₃, a C₁₋₄-aliphatic residue, C(═O)—OH, and a C₃₋₆ cycloaliphatic residue, wherein the C₁₋₄-aliphatic residue in each case may be unsubstituted or mono- or polysubstituted with at least one substituent selected from the group consisting of F, Cl, OH, OCF₃, CF₃ and an unsubstituted O—C₁₋₄-aliphatic residue, and wherein the C₃₋₆ cycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, CF₃, a C₁₋₄-aliphatic residue and C(═O)—OH, and wherein the 3 to 10 membered heterocycloaliphatic residue formed by R⁴ and R⁵ together with the nitrogen atom connecting them may optionally be condensed with a C₃₋₆ cycloaliphatic residue, preferably cyclopropyl, cyclobutyl or cyclopentyl, or a 4 to 7 membered heterocycloaliphatic residue, preferably oxetanyl or oxiranyl, wherein the C₃₋₆ cycloaliphatic residue or the 4 to 7 membered heterocycloaliphatic residue condensed in this way can for their part be respectively unsubstituted or mono- or polysubstituted with at least one substituent selected from the group consisting of F, Cl, Br, I, ═O, OH, an O—C₁₋₄ aliphatic residue, OCF₃, SCF₃, CF₃, CN, a C₁₋₄-aliphatic residue, C(═O)—OH, C(═O)—CH₃, C(═O)—C₂H₅, C(═O)—O—CH₃ and C(═O)—O—C₂H₅.

In further embodiments, R⁶ denotes a C₂₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a C(═O)—O—C₁₋₄-aliphatic residue, a S—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue. on the condition that if R⁶ denotes a 3 to 10 membered heterocycloaliphatic residue, the 3 to 10 membered heterocycloaliphatic residue is linked via a carbon atom, or R⁶ denotes S—R⁷ or O—R⁸ wherein R⁷ and R⁸ in each case denote a C₁₋₈-aliphatic residue, unsubstituted or mono- or polysubstituted with at least one substituent selected from the group consisting of F, Cl, Br, I, OH, —O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, CF₃, a C(═O)—O—C₁₋₄-aliphatic residue, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or in each case denote a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may be bridged via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue, on the condition that if R⁷ or R⁸ denotes a 3 to 10 membered heterocycloaliphatic residue, the 3 to 10 membered heterocycloaliphatic residue is linked via a carbon atom, or R⁶ denotes N(R⁹R¹⁰), wherein R⁹ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, CF₃, a C(═O)—O—C₁₋₄-aliphatic residue, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case be bridged, preferably is bridged, via a C₁₋₈ aliphatic group, preferably a C₁₋₄ aliphatic group, which in turn may be unsubstituted or mono- or polysubstituted with at least one substituent selected from the group consisting of F, Cl, Br, I, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, and a C₁₋₄-aliphatic residue, on the condition that if Ro denotes a 3 to 10 membered heterocycloaliphatic residue, the 3 to 10 membered heterocycloaliphatic residue is linked via a carbon atom, and R¹⁰ denotes a C₁₋₆-aliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or R⁹ and R¹⁰ form together with the nitrogen atom connecting them a 3 to 10 membered heterocycloaliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, and wherein the 3 to 10 membered heterocycloaliphatic residue formed by R⁹ and R¹⁰ together with the nitrogen atom connecting them may optionally be condensed with aryl or heteroaryl, preferably with phenyl or pyridyl, wherein the aryl or heteroaryl residues condensed in this way can for their part be respectively unsubstituted or mono- or polysubstituted with at least one substituent selected from the group consisting of F, Cl, Br, I, NO₂, OH, an O—C₁₋₄ aliphatic residue, OCF₃, SH, SCF₃, a S—C₁₋₄ aliphatic residue, CF₃,

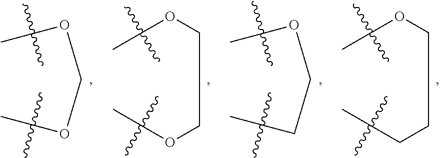 [SMILES: *OCO* ; *OCCO* ; *CCO* ; *CCCO*]

CN, a C₁₋₄-aliphatic residue, C(═O)—OH, residue, benzyl, phenyl, thienyl, and pyridyl, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, and wherein benzyl, phenyl, thienyl, and pyridyl, may in each case may be unsubstituted or mono- or polysubstituted with at least one substituent selected from the group consisting of F, Cl, Br, I, NO₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, and C(═O)—OH.

In further embodiments, R⁶ denotes a C₂₋₆-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue wherein the C₁₋₄-aliphatic residue in each case is unsubstituted, or denotes a C₃₋₁₀-cycloaliphatic residue, or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)-O—C₁₋₄-aliphatic residue, CF₃, and a C₁₋₄-aliphatic residue, wherein the C₁₋₄-aliphatic residue in each case may be unsubstituted or mono- or polysubstituted with OH or an unsubstituted O—C₁₋₄-aliphatic residue, and wherein the C₃₋₁₀-cycloaliphatic residue or the 3 to 10 membered heterocycloaliphatic residue may in each case optionally bridged via a unsubstituted C₁₋₄ aliphatic group, on the condition that if R⁶ denotes a 3 to 10 membered heterocycloaliphatic residue, the 3 to 10 membered heterocycloaliphatic residue is linked via a carbon atom, or R⁶ denotes S—R⁷ or O—R⁸ wherein R⁷ and R⁸ in each case denote a C₁₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, an NH(C₁₋₄ aliphatic residue), an N(C₁₋₄ aliphatic residue)₂, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or in each case denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue in each case may be bridged via an unsubstituted C₁₋₈ aliphatic group, on the condition that if R⁷ or R⁸ denotes a 3 to 10 membered heterocycloaliphatic residue, the 3 to 10 membered heterocycloaliphatic residue is linked via a carbon atom, or R⁶ denotes N(R⁹R¹⁰), wherein R⁹ denotes a C₁₋₈-aliphatic residue, unsubstituted or mono- or polysubstituted with at least one substituent selected from the group consisting of F, Cl, Br, I, OH, —O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue is in each case bridged via a unsubstituted C₁₋₈ aliphatic group, preferably an unsubstituted C₁₋₄ aliphatic group, on the condition that if Ro denotes a 3 to 10 membered heterocycloaliphatic residue, the 3 to 10 membered heterocycloaliphatic residue is linked via a carbon atom, and R¹⁰ denotes an unsubstituted C₁₋₄-aliphatic residue, or R⁹ and R¹⁰ form together with the nitrogen atom connecting them a 3 to 6 membered heterocycloaliphatic residue, unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, and wherein the 3 to 6 membered heterocycloaliphatic residue formed by R⁹ and R¹⁰ together with the nitrogen atom connecting them may optionally be condensed with phenyl or pyridyl, wherein the phenyl or pyridyl residues condensed in this way can for their part be respectively unsubstituted or mono- or polysubstituted with at least one substituent selected from the group consisting of F, Cl, Br, I, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, residue, benzyl, phenyl, and pyridyl, wherein the C₁₋₄-aliphatic residue in each case may be unsubstituted or mono- or polysubstituted with at least one substituent selected from the group consisting of F, C₁, OH, and an unsubstituted O—C₁₋₄-aliphatic residue, and wherein benzyl, phenyl, and pyridyl, may in each case may be unsubstituted or mono- or polysubstituted with at least one substituent selected from the group consisting of F, Cl, Br, I, OCH₃, OCF₃, O—CH₂—OH, OCH₂—O—CH₃, SH, SCF₃, CF₃, and a C₁₋₄-aliphatic residue.

In further embodiments, R¹ represents the partial structure:

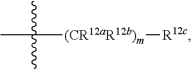

wherein: m is 0, 1 or 2, and R^{12a} and R^{12b} each independently of one another represent H, F, OH, CH₃ or OCH₃, or together denote ═O, R^{12c} denotes a C₁₋₄ aliphatic residue, unsubstituted or mono- or polysubstituted with at least one substituent selected from the group consisting of F, Cl, Br, I, CN, OH, S(═O)₂—CH₃, an unsubstituted O—C₁₋₄ aliphatic residue, and CF₃, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0 or 2, and R^{12a} and R^{12b} each independently of one another represent H, F, OH, CH₃ or OCH₃; and R^{12c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, OCF₂H, CH₂—OH, CH₂—OCH₃, S(═O)₂—CH₃, SCF₃, NO₂, N(CH₃)₂,

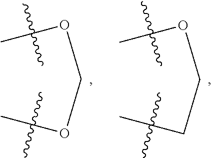 [SMILES: *OCO* ; *CCO*]

CF₃, CN, a C₁₋₄-aliphatic residue, C(═O)—CH₃, C(═O)—C₂H₅, C(═O)—O—CH₃, C(═O)—O—C₂H₅ and phenyl, wherein phenyl may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, R² represents F; Cl; Br; I; CN; CF₃; NO₂; OCF₃; SCF₃; methyl; ethyl; n-propyl; iso-propyl; n-butyl; sec.-butyl; tert.-butyl; CH₂—OH; CH₂—O—CH₃; CH₂—CH₂—OH; CH₂ CH₂—OCH₃; O-methyl; O-ethyl; O—(CH₂)₂—O—CH₃; O—(CH₂)₂—OH; S-Methyl; S-Ethyl; cyclopropyl, cyclobutyl, cyclopentyl, or cyclohexyl, R³ represents H; F; Cl; Br; I; CN; CF₃; SCF₃; NO₂; OCF₃; methyl; ethyl; n-propyl; iso-propyl; n-butyl; sec.-butyl; tert.-butyl; O-methyl; O-ethyl; O—(CH₂)₂—O—CH₃; O—(CH₂)₂—OH; S-Methyl; or S-Ethyl, R⁴ represents the partial structure (T2)

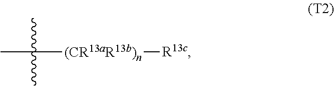

wherein n denotes 0, 1, 2 or 3, R^{13a} and R^{13b} each independently of one another represent H, F, CH₃ or OCH₃, or together denote ═O, R^{13c} denotes a C₁₋₄ aliphatic residue, unsubstituted or mono- or polysubstituted with at least one substituent selected from the group consisting of F, Cl, Br, I, —O, an unsubstituted O—C₁₋₄ aliphatic residue, and CF₃, or denotes cyclopropyl, cyclobutyl, cyclopentyl or cyclohexyl, pyrrolidinyl, morpholinyl, piperazinyl, piperidinyl or tetrahydropyranyl, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, and a C₁₋₄-aliphatic residue, R⁵ denotes H, methyl or ethyl, C₂H₄OCH₃ or C₃H₆OCH₃, or R⁴ and R⁵ form together with the nitrogen atom connecting them a morpholinyl, piperidinyl, pyrrolidinyl, azetidinyl, oxazepanyl, tetrahydroquinolinyl, tetrahydroisoquinolinyl, thiomorpholinyl, azepanyl,

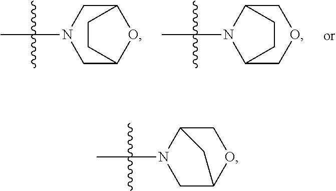 [SMILES: *N1CC2CCC(C1)O2 ; *N1C2CCC1COC2 ; *N1CC2CC1CO2]

tetrahydroimidazo[1,2-a]pyrazinyl, octahydropyrrolo[1,2-a]pyrazinyl,

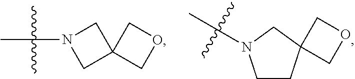 [SMILES: *N1CC2(COC2)C1 ; *N1CCC2(COC2)C1]

dihydroindolinyl, or dihydroisoindolyl, in each case unsubstituted or mono- or polysubstituted with at least one substituent selected from the group consisting of F, Cl, OH, ═O, C(═O)—OH, O-methyl, O-ethyl, OCF₃, SCF₃, CF₃, C(═O)—CH₃, C(═O)—OCH₃, CH₂CF₃, CH₂OH, CH₂—OCH₃, CH₂CH₂—OCH₃, methyl, ethyl, n-propyl, 2-propyl, cyclopropyl, and cyclobutyl, R⁶ denotes ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), CH₂—OCH₃, C₂H₄—OCH₃, C₃H₆—OCH₃, cyclopropyl, cyclobutyl, or tetrahydropyranyl, ethenyl or propenyl (—CH₂CH═CH₂, —CH═CH—CH₃, —C((═CH₂)—CH₃), in each case unsubstituted, or R⁶ denotes S—R⁷ or O—R⁸ wherein R⁷ and R⁸ in each case denote methyl, ethyl, n-propyl, 2-propyl, n-butyl, isobutyl, sec.-butyl, tert.-butyl, n-pentyl, isopentyl, neopentyl, or n-hexyl, in each case unsubstituted or mono- or polysubstituted with at least one substituent selected from the group consisting of F, Cl, OH, N(C₁₋₄ aliphatic residue) and an O—C₁₋₄-aliphatic residue, or in each case denote CH₂-cyclopropyl or oxetanyl, wherein the C₁₋₄-aliphatic residue in each case is unsubstituted, or R⁶ denotes N(R⁹R¹⁰), wherein R⁹ denotes methyl, C(═O)—CH₃, ethyl, n-propyl, 2-propyl, n-butyl, isobutyl, sec.-butyl, tert.-butyl, n-pentyl, isopentyl, neopentyl, or n-hexyl, R¹⁰ denotes methyl or ethyl, or R⁹ and R¹⁰ form together with the nitrogen atom connecting them a morpholinyl, piperidinyl, pyrrolidinyl, or azetidinyl, in each case unsubstituted.

In further embodiments, the Kv7 channel activator is selected from the group consisting of: N-[(3,5-Difluoro-phenyl)-methyl]-2-ethylsulfanyl-4-methyl-6-morpholin-4-yl-pyridine-3-carboxylic acid amide; Ethylsulfanyl-N-[(3-fluorophenyl)-methyl]-4-methyl-6-morpholin-4-yl-pyridine-3-carboxylic acid amide; N-[(3,5-Difluoro-phenyl)-methyl]-2-methoxy-4-methyl-6-morpholin-4-yl-pyridine-3-carboxylic acid amide; 2-Ethylsulfanyl-N-[(4-fluorophenyl)-methyl]-4-methyl-6-morpholin-4-yl-pyridine-3-carboxylic acid amide; 2-Ethylsulfanyl-N-[(3-fluorophenyl)-methyl]-4-methyl-6-[methyl-(tetrahydro-pyran-2-yl-methyl)-amino]-pyridine-3-carboxylic acid amide; 2-Ethylsulfanyl-N-[(3-fluorophenyl)-methyl]-6-(3-methoxy-azetidin-1-yl)-4-methyl-pyridine-3-carboxylic acid amide; 2-Ethylsulfanyl-N-[(3-fluorophenyl)-methyl]-6-(3-hydroxy-azetidin-1-yl)-4-methyl-pyridine-3-carboxylic acid amide; 2-Ethylsulfanyl-N-[(3-fluorophenyl)-methyl]-6-[(4-fluorophenyl)-methylamino]-4-methyl-pyridine-3-carboxylic acid amide; N-[(3-Fluorophenyl)-methyl]-4-methyl-6-morpholin-4-yl-2-[(E)-prop-1-enyl]-pyridine-3-carboxylic acid amide; N-[(3-Fluorophenyl)methyl]-4-methyl-6-morpholin-4-yl-2-propyl-pyridine-3-carboxylic acid amide; 2-Ethylsulfanyl-N-[(3-fluorophenyl)-methyl]-6-morpholin-4-yl-4-(trifluoromethyl)-pyridine-3-carboxylic acid amide; N-[(3-Fluorophenyl)methyl]-4-methyl-2,6-dimorpholin-4-yl-pyridine-3-carboxylic acid amide; 1-[6-Ethylsulfanyl-5-[(3-fluorophenyl)-methyl-carbamoyl]-4-methyl-pyridin-2-yl]-piperidine-4-carboxylic acid methyl ester; 1-[6-Ethylsulfanyl-5-[(3-fluorophenyl)-methyl-carbamoyl]-4-methyl-pyridin-2-yl]-piperidine-4-carboxylic acid; 2-Ethylsulfanyl-N-[(3-fluorophenyl)-methyl]-6-(4-hydroxy-piperidin-1-yl)-4-methyl-pyridine-3-carboxylic acid amide; 2-Ethylsulfanyl-N-[(3-fluorophenyl)-methyl]-4-methyl-6-(4-oxo-piperidin-1-yl)-pyridine-3-carboxylic acid amide; 2-Ethylsulfanyl-N-[(4-fluoro-2-methoxy-phenyl)-methyl]-4-methyl-6-morpholin-4-yl-pyridine-3-carboxylic acid amide; 2-Ethylsulfanyl-N-[(4-fluoro-2-hydroxy-phenyl)-methyl]-4-methyl-6-morpholin-4-yl-pyridine-3-carboxylic acid amide; N-[(3-Fluorophenyl)methyl]-2-methoxy-4-methyl-6-morpholin-4-yl-pyridine-3-carboxylic acid amide; N-[(3-Fluorophenyl)-methyl]-2-(2-methoxy-ethoxy)-4-methyl-6-morpholin-4-yl-pyridine-3-carboxylic acid amide; 2-Ethyl-N-[(3-fluorophenyl)-methyl]-4-methyl-6-morpholin-4-yl-pyridine-3-carboxylic acid amide; N-[(3-Fluorophenyl)methyl]-2-isopropyl-4-methyl-6-morpholin-4-yl-pyridine-3-carboxylic acid amide; N-[(3-Fluorophenyl)methyl]-4-methyl-6-morpholin-4-yl-2-pyrrolidin-1-yl-pyridine-3-carboxylic acid amide; 2-Ethylsulfanyl-N-[(3-fluorophenyl)-methyl]-4-methyl-6-pyrrolidin-1-yl-pyridine-3-carboxylic acid amide; 2-Ethylsulfanyl-N-[(3-fluorophenyl)-methyl]-4-methyl-6-(1,2,3,4-tetrahydro-isoquinolin-2-yl)-pyridine-3-carboxylic acid amide; 2-Ethylsulfanyl-N-[(3-fluorophenyl)-methyl]-4-methyl-6-[6-(trifluoromethyl)-1,2,3,4-tetrahydro-isoquinolin-2-yl]-pyridine-3-carboxylic acid amide; N-[(4-Fluorophenyl)methyl]-4-methyl-6-morpholin-4-yl-2-[(E)-prop-1-enyl]-pyridine-3-carboxylic acid amide; N-[(4-Fluorophenyl)-methyl]-4-methyl-6-morpholin-4-yl-2-propyl-pyridine-3-carboxylic acid amide; 2-Ethylsulfanyl-N-[(3-fluorophenyl)-methyl]-6-(3-methoxy-pyrrolidin-1-yl)-4-methyl-pyridine-3-carboxylic acid amide; 2-Ethylsulfanyl-N-[(3-fluorophenyl)-methyl]-4-methyl-6-(4-methyl-piperazin-1-yl)-pyridine-3-carboxylic acid amide; 2-Ethylsulfanyl-N-[(3-fluorophenyl)-methyl]-4-methyl-6-piperidin-1-yl-pyridine-3-carboxylic acid amide; 6-Dimethylamino-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-4-methyl-6-methylamino-pyridine-3-carboxylic acid amide; 2-Ethylsulfanyl-N-[(3-fluorophenyl)-methyl]-6-(2-methoxy-ethyl-methyl-amino)-4-methyl-pyridine-3-carboxylic acid amide; 2-Ethylsulfanyl-N-[(3-fluorophenyl)-methyl]-6-(2-methoxy-ethylamino)-4-methyl-pyridine-3-carboxylic acid amide; N-[(3-Fluorophenyl)-methyl]-2-(isopropylsulfanyl)-4-methyl-6-morpholin-4-yl-pyridine-3-carboxylic acid amide; 2-Ethoxy-N-[(3-fluorophenyl)-methyl]-4-methyl-6-morpholin-4-yl-pyridine-3-carboxylic acid amide; N-[(4-Fluorophenyl)-methyl]-2-methoxy-4-methyl-6-morpholin-4-yl-pyridine-3-carboxylic acid amide; N-[(3-Fluorophenyl)-methyl]-4-methyl-2-methylsulfanyl-6-morpholin-4-yl-pyridine-3-carboxylic acid amide; N-[(3,4-Difluoro-phenyl)-methyl]-2-ethylsulfanyl-4-methyl-6-morpholin-4-yl-pyridine-3-carboxylic acid amide; 2-Ethylsulfanyl-4-methyl-N-(3-methyl-butyl)-6-morpholin-4-yl-pyridine-3-carboxylic acid amide; N-(Cyclopentyl-methyl)-2-ethylsulfanyl-4-methyl-6-morpholin-4-yl-pyridine-3-carboxylic acid amide; N-(2-Cyclopentyl-ethyl)-2-ethylsulfanyl-4-methyl-6-morpholin-4-yl-pyridine-3-carboxylic acid amide; 2-Ethylsulfanyl-N-[(6-fluoro-pyridin-2-yl)-methyl]-4-methyl-6-morpholin-4-yl-pyridine-3-carboxylic acid amide; 2-Ethylsulfanyl-N-[(5-fluoro-pyridin-2-yl)-methyl]-4-methyl-6-morpholin-4-yl-pyridine-3-carboxylic acid amide; N-(2,2-Dimethyl-propyl)-2-ethylsulfanyl-4-methyl-6-morpholin-4-yl-pyridine-3-carboxylic acid amide; 2-Ethylsulfanyl-N-[(3-fluorophenyl)-methyl]-4-methyl-6-(2-methyl-morpholin-4-yl)-pyridine-3-carboxylic acid amide; 2-Ethylsulfanyl-N-[(3-fluorophenyl)-methyl]-6-(4-methoxy-piperidin-1-yl)-4-methyl-pyridine-3-carboxylic acid amide; 2-Ethylsulfanyl-4-methyl-6-morpholin-4-yl-N-[(2-phenyl-phenyl)-methyl]-pyridine-3-carboxylic acid amide; 2-Ethylsulfanyl-4-methyl-6-morpholin-4-yl-N-[[2-(trifluoromethyl)-phenyl]-methyl]-pyridine-3-carboxylic acid amide; 2-Ethylsulfanyl-N-[(3-fluorophenyl)-methyl]-6-[(4-fluorophenyl)-methyl-methyl-amino]-4-methyl-pyridine-3-carboxylic acid amide; 2-Ethylsulfanyl-4-methyl-6-morpholin-4-yl-N-(3-phenyl-propyl)-pyridine-3-carboxylic acid amide; 2-Ethylsulfanyl-4-methyl-6-morpholin-4-yl-N-phenethyl-pyridine-3-carboxylic acid amide; N-Benzyl-2-ethylsulfanyl-4-methyl-6-morpholin-4-yl-pyridine-3-carboxylic acid amide N-[(3-Fluorophenyl)-methyl]-4-methyl-6-morpholin-4-yl-2-(propylsulfanyl)-pyridine-3-carboxylic acid amide; 2-(Butylsulfanyl)-N-[(3-fluorophenyl)-methyl]-4-methyl-6-morpholin-4-yl-pyridine-3-carboxylic acid amide; 2-Ethylsulfanyl-5-fluoro-N-[(3-fluorophenyl)-methyl]-4-methyl-6-morpholin-4-yl-pyridine-3-carboxylic acid amide; 2-Ethylsulfanyl-4-methyl-6-morpholin-4-yl-N-[[3-(trifluoromethyl)phenyl]-methyl]-pyridine-3-carboxylic acid amide; 2-Ethylsulfanyl-4-methyl-6-morpholin-4-yl-N-[[4-(trifluoromethyl)-phenyl]-methyl]-pyridine-3-carboxylic acid amide; 2-Ethylsulfanyl-N-[(3-fluorophenyl)-methyl]-4-methyl-6-[methyl-(tetrahydro-pyran-4-yl-methyl)-amino]-pyridine-3-carboxylic acid amide; N-[(3-Fluorophenyl)-methyl]-4-methyl-2-(2-methyl-propylsulfanyl)-6-morpholin-4-yl-pyridine-3-carboxylic acid amide; N-[(3-Fluorophenyl)-methyl]-2-(2-methoxy-ethylsulfanyl)-4-methyl-6-morpholin-4-yl-pyridine-3-carboxylic acid amide; 2-Ethoxy-N-[(4-fluorophenyl)-methyl]-4-methyl-6-morpholin-4-yl-pyridine-3-carboxylic acid amide; 2-Dimethylamino-N-[(3-fluorophenyl)-methyl]-4-methyl-6-morpholin-4-yl-pyridine-3-carboxylic acid amide; 6-(2,6-Dimethyl-morpholin-4-yl)-2-ethylsulfanyl-N-[(3-fluorophenyl)-methyl]-4-methyl-pyridine-3-carboxylic acid amide; N-(4,4-Dimethyl-pentyl)-2-ethylsulfanyl-4-methyl-6-morpholin-4-yl-pyridine-3-carboxylic acid amide; 2-Ethylsulfanyl-4-methyl-6-morpholin-4-yl-N-(2-tetrahydro-pyran-2-yl-ethyl)-pyridine-3-carboxylic acid amide; 2-Ethylsulfanyl-4-methyl-6-morpholin-4-yl-N-(tetrahydro-pyran-2-yl-methyl)-pyridine-3-carboxylic acid amide; 2-Ethylsulfanyl-N-[(3-fluorophenyl)-methyl]-4-methyl-6-(4-methyl-piperidin-1-yl)-pyridine-3-carboxylic acid amide; 2-Ethylsulfanyl-N-[[2-(4-fluorophenyl)-phenyl]methyl]-4-methyl-6-morpholin-4-yl-pyridine-3-carboxylic acid amide; 2-[6-Ethylsulfanyl-5-[[(3-fluorophenyl)-methyl-carbamoyl]-4-methyl-pyridin-2-yl]-methyl-amino]-acetic acid ethyl ester; 6-(4-Cyclopropyl-piperazin-1-yl)-2-ethylsulfanyl-N-[(3-fluorophenyl)-methyl]-4-methyl-pyridine-3-carboxylic acid amide; 6-(4,4-Dimethyl-piperidin-1-yl)-2-ethylsulfanyl-N-[(3-fluorophenyl)-methyl]-4-methyl-pyridine-3-carboxylic acid amide; 2-Ethylsulfanyl-4-methyl-6-morpholin-4-yl-N-[[4-(trifluoromethylsulfanyl)-phenyl]-methyl]pyridine-3-carboxylic acid amide; N-(Cyclohexyl-methyl)-2-ethylsulfanyl-4-methyl-6-morpholin-4-yl-pyridine-3-carboxylic acid amide; 2-Ethylsulfanyl-N-(2-methoxy-ethyl)-4-methyl-6-morpholin-4-yl-pyridine-3-carboxylic acid amide; 2-Ethylsulfanyl-N-(3-methoxy-propyl)-4-methyl-6-morpholin-4-yl-pyridine-3-carboxylic acid amide; 2-Ethylsulfanyl-4-methyl-N-(4-methyl-pentyl)-6-morpholin-4-yl-pyridine-3-carboxylic acid amide; N-Butyl-2-ethylsulfanyl-4-methyl-6-morpholin-4-yl-pyridine-3-carboxylic acid amide; 2-Ethylsulfanyl-4-methyl-6-morpholin-4-yl-N-pentyl-pyridine-3-carboxylic acid amide; 2-Ethylsulfanyl-N-[[4-fluoro-3-(trifluoromethyl)-phenyl]methyl]-4-methyl-6-morpholin-4-yl-pyridine-3-carboxylic acid amide; N-(2-tert-Butoxy-ethyl)-2-ethylsulfanyl-4-methyl-6-morpholin-4-yl-pyridine-3-carboxylic acid amide; 2-Ethylsulfanyl-4-methyl-6-morpholin-4-yl-N-(4,4,4-trifluoro-butyl)-pyridine-3-carboxylic acid amide; 2-Ethylsulfanyl-N-[[4-fluoro-2-(4-fluorophenyl)-phenyl]methyl]-4-methyl-6-morpholin-4-yl-pyridine-3-carboxylic acid amide; N-(4,4-Dimethyl-pentyl)-2-methoxy-4-methyl-6-morpholin-4-yl-pyridine-3-carboxylic acid amide; N-[(3,4-Difluoro-phenyl)-methyl]-2-methoxy-4-methyl-6-morpholin-4-yl-pyridine-3-carboxylic acid amide; 2-Methoxy-4-methyl-6-morpholin-4-yl-N-[(2-phenyl-phenyl)-methyl]-pyridine-3-carboxylic acid amide; N-(4,4-Dimethyl-pentyl)-2-ethoxy-4-methyl-6-morpholin-4-yl-pyridine-3-carboxylic acid amide; N-[(3,5-Difluoro-phenyl)-methyl]-2-ethoxy-4-methyl-6-morpholin-4-yl-pyridine-3-carboxylic acid amide; N-[(3,4-Difluoro-phenyl)-methyl]-2-ethoxy-4-methyl-6-morpholin-4-yl-pyridine-3-carboxylic acid amide; 2-Ethoxy-4-methyl-6-morpholin-4-yl-N-[(2-phenyl-phenyl)-methyl]-pyridine-3-carboxylic acid amide; 2-Ethylsulfanyl-N-[[3-fluoro-5-(trifluoromethyl)-phenyl]methyl]-4-methyl-6-morpholin-4-yl-pyridine-3-carboxylic acid amide; 2-Ethylsulfanyl-N-[[2-fluoro-3-(trifluoromethyl)-phenyl]methyl]-4-methyl-6-morpholin-4-yl-pyridine-3-carboxylic acid amide; 2-Ethylsulfanyl-N-[[2-fluoro-5-(trifluoromethyl)-phenyl]methyl]-4-methyl-6-morpholin-4-yl-pyridine-3-carboxylic acid amide; 2-Ethylsulfanyl-N-[(3-fluorophenyl)-methyl]-4-methyl-6-([1,4]oxazepan-4-yl)-pyridine-3-carboxylic acid amide; 2-Ethylsulfanyl-4-methyl-6-morpholin-4-yl-N-[[4-(trifluoromethyloxy)-phenyl]-methyl]pyridine-3-carboxylic acid amide; N-[(3-Fluorophenyl)-methyl]-2-methoxy-4-methyl-6-([1,4]oxazepan-4-yl)-pyridine-3-carboxylic acid amide; 2-Ethoxy-N-[(3-fluorophenyl)-methyl]-4-methyl-6-([1,4]oxazepan-4-yl)-pyridine-3-carboxylic acid amide; N-[(2,3-Difluoro-phenyl)-methyl]-2-ethylsulfanyl-4-methyl-6-morpholin-4-yl-pyridine-3-carboxylic acid amide; N-[(2,5-Difluoro-phenyl)-methyl]-2-ethylsulfanyl-4-methyl-6-morpholin-4-yl-pyridine-3-carboxylic acid amide; N-[(3-Cyano-phenyl)-methyl]-2-ethylsulfanyl-4-methyl-6-morpholin-4-yl-pyridine-3-carboxylic acid amide; 2-Ethylsulfanyl-N-(2-isopropoxy-ethyl)-4-methyl-6-morpholin-4-yl-pyridine-3-carboxylic acid amide; N-(3,3-Dimethyl-butyl)-2-ethylsulfanyl-4-methyl-6-morpholin-4-yl-pyridine-3-carboxylic acid amide; N-(3-Cyclopentyl-propyl)-2-ethylsulfanyl-4-methyl-6-morpholin-4-yl-pyridine-3-carboxylic acid amide; N-(2-Cyclohexyl-ethyl)-2-ethylsulfanyl-4-methyl-6-morpholin-4-yl-pyridine-3-carboxylic acid amide; N-[(2,4-Difluoro-phenyl)-methyl]-2-ethylsulfanyl-4-methyl-6-morpholin-4-yl-pyridine-3-carboxylic acid amide; 2-Ethylsulfanyl-N-[3-(4-fluorophenyl)-propyl]-4-methyl-6-morpholin-4-yl-pyridine-3-carboxylic acid amide; 2-Ethylsulfanyl-4-methyl-6-morpholin-4-yl-N-(3-pyridin-2-yl-propyl)-pyridine-3-carboxylic acid amide; 2-Butoxy-N-[(3-fluorophenyl)-methyl]-4-methyl-6-morpholin-4-yl-pyridine-3-carboxylic acid amide; N-[(3-Fluorophenyl)-methyl]-4-methyl-6-morpholin-4-yl-2-propoxy-pyridine-3-carboxylic acid amide; 2-Ethylsulfanyl-N-[(3-fluorophenyl)-methyl]-4-methyl-6-(3-oxo-azetidin-1-yl)-pyridine-3-carboxylic acid amide; 2-Ethylsulfanyl-N-[3-(3-fluorophenyl)-propyl]-4-methyl-6-morpholin-4-yl-pyridine-3-carboxylic acid amide; 2-Ethylsulfanyl-4-methyl-6-morpholin-4-yl-N-(3-pyridin-3-yl-propyl)-pyridine-3-carboxylic acid amide; 2-Ethylsulfanyl-4-methyl-6-morpholin-4-yl-N-(3-pyridin-4-yl-propyl)-pyridine-3-carboxylic acid amide; N-(5,5-Dimethyl-hexyl)-2-ethylsulfanyl-4-methyl-6-morpholin-4-yl-pyridine-3-carboxylic acid amide; 2-Methoxy-4-methyl-6-morpholin-4-yl-N-[[4-(trifluoromethyl)-phenyl]-methyl]-pyridine-3-carboxylic acid amide; 2-Ethylsulfanyl-N-[(3-fluorophenyl)-methyl]-4-methyl-6-[methyl-(pyridin-4-yl-methyl)-amino]-pyridine-3-carboxylic acid amide; 2-Ethylsulfanyl-N-[(3-fluorophenyl)-methyl]-4-methyl-6-[methyl-(pyridin-3-yl-methyl)-amino]-pyridine-3-carboxylic acid amide; 2-Ethylsulfanyl-6-[(4-fluoro-benzoyl)-methyl-amino]-N-[(3-fluorophenyl)-methyl]-4-methyl-pyridine-3-carboxylic acid amide; 2-Ethylsulfanyl-N-[(3-fluorophenyl)-methyl]-4-methyl-6-[methyl-(pyridin-2-yl-methyl)-amino]-pyridine-3-carboxylic acid amide; 2-Ethylsulfanyl-N-[(3-fluorophenyl)-methyl]-4-methyl-6-(pyridin-3-yl-methylamino)-pyridine-3-carboxylic acid amide; 6-(Acetyl-methyl-amino)-2-ethylsulfanyl-N-[(3-fluorophenyl)-methyl]-4-methyl-pyridine-3-carboxylic acid amide; N-[(4-Chlorophenyl)-methyl]-2-ethylsulfanyl-4-methyl-6-morpholin-4-yl-pyridine-3-carboxylic acid amide; N-[(3-Chlorophenyl)-methyl]-2-ethylsulfanyl-4-methyl-6-morpholin-4-yl-pyridine-3-carboxylic acid amide; 6-[Bis(2-methoxy-ethyl)-amino]-2-ethylsulfanyl-N-[(3-fluorophenyl)-methyl]-4-methyl-pyridine-3-carboxylic acid amide; 2-(Ethyl-methyl-amino)-N-[(3-fluorophenyl)-methyl]-4-methyl-6-morpholin-4-yl-pyridine-3-carboxylic acid amide; 2-Ethylsulfanyl-N-[(3-fluorophenyl)-methyl]-6-(3-methoxy-propyl-methyl-amino)-4-methyl-pyridine-3-carboxylic acid amide; 2-Ethylsulfanyl-N-[3-(2-fluorophenyl)-propyl]-4-methyl-6-morpholin-4-yl-pyridine-3-carboxylic acid amide; 2-Ethylsulfanyl-4-methyl-6-morpholin-4-yl-N-[[3-(trifluoromethyloxy)-phenyl]-methyl]pyridine-3-carboxylic acid amide; 2-Ethylsulfanyl-N-[[3-(methoxymethyl)-phenyl]methyl]-4-methyl-6-morpholin-4-yl-pyridine-3-carboxylic acid amide; 2-Ethoxy-4-methyl-6-morpholin-4-yl-N-[[4-(trifluoromethyl)-phenyl]-methyl]-pyridine-3-carboxylic acid amide; 2-Ethoxy-4-methyl-6-morpholin-4-yl-N-(4,4,4-trifluoro-butyl)-pyridine-3-carboxylic acid amide; N-(1,3-Benzodioxol-5-yl-methyl)-2-ethylsulfanyl-4-methyl-6-morpholin-4-yl-pyridine-3-carboxylic acid amide; 2-Ethylsulfanyl-N-[[2-fluoro-4-(trifluoromethyl)-phenyl]methyl]-4-methyl-6-morpholin-4-yl-pyridine-3-carboxylic acid amide; 6-(Azepan-1-yl)-2-ethylsulfanyl-N-[(3-fluorophenyl)-methyl]-4-methyl-pyridine-3-carboxylic acid amide; 2-Ethylsulfanyl-N-[(4-methoxyphenyl)-methyl]-4-methyl-6-morpholin-4-yl-pyridine-3-carboxylic acid amide; (2S)-2-Ethylsulfanyl-N-[(3-fluorophenyl)-methyl]-4-methyl-6-(2-methyl-morpholin-4-yl)-pyridine-3-carboxylic acid amide; (2R)-2-Ethylsulfanyl-N-[(3-fluorophenyl)-methyl]-4-methyl-6-(2-methyl-morpholin-4-yl)-pyridine-3-carboxylic acid amide; 2-Methoxy-4-methyl-6-morpholin-4-yl-N-(4,4,4-trifluoro-butyl)-pyridine-3-carboxylic acid amide; N-(3-Cyclopropyl-propyl)-2-ethylsulfanyl-4-methyl-6-morpholin-4-yl-pyridine-3-carboxylic acid amide; 2-Ethylsulfanyl-N-[[3-fluoro-4-(trifluoromethyl)-phenyl]methyl]-4-methyl-6-morpholin-4-yl-pyridine-3-carboxylic acid amide; 2-Ethylsulfanyl-N-[(3-fluorophenyl)-methyl]-4-methyl-6-(3-oxo-piperazin-1-yl)-pyridine-3-carboxylic acid amide; 6-(4-Acetyl-piperazin-1-yl)-2-ethylsulfanyl-N-[(3-fluorophenyl)-methyl]-4-methyl-pyridine-3-carboxylic acid amide; N-[(4-Cyano-phenyl)-methyl]-2-ethylsulfanyl-4-methyl-6-morpholin-4-yl-pyridine-3-carboxylic acid amide; 2-Ethylsulfanyl-N-[[4-(methoxymethyl)-phenyl]methyl]-4-methyl-6-morpholin-4-yl-pyridine-3-carboxylic acid amide; 2-Ethylsulfanyl-N-[[3-fluoro-4-(methoxymethyl)-phenyl]methyl]-4-methyl-6-morpholin-4-yl-pyridine-3-carboxylic acid amide; N-[(4-Dimethylaminophenyl)-methyl]-2-ethylsulfanyl-4-methyl-6-morpholin-4-yl-pyridine-3-carboxylic acid amide; 2-Ethylsulfanyl-N-[[4-fluoro-3-(methoxymethyl)-phenyl]methyl]-4-methyl-6-morpholin-4-yl-pyridine-3-carboxylic acid amide; 2-Ethylsulfanyl-N-[(3-fluorophenyl)-methyl]-4-methyl-6-(4-methyl-3-oxo-piperazin-1-yl)-pyridine-3-carboxylic acid amide; 2-Ethylsulfanyl-N-[(3-fluorophenyl)-methyl]-4-methyl-6-(6-oxa-2-azaspiro[3.3]heptan-2-yl)-pyridine-3-carboxylic acid amide; N-(4,4-Dimethyl-pentyl)-4-methyl-2-methylsulfanyl-6-morpholin-4-yl-pyridine-3-carboxylic acid amide; 4-Methyl-2-methylsulfanyl-6-morpholin-4-yl-N-(4,4,4-trifluoro-butyl)-pyridine-3-carboxylic acid amide; N-[(4-Fluorophenyl)-methyl]-4-methyl-2-methylsulfanyl-6-morpholin-4-yl-pyridine-3-carboxylic acid amide; N-[(3,4-Difluoro-phenyl)-methyl]-4-methyl-2-methylsulfanyl-6-morpholin-4-yl-pyridine-3-carboxylic acid amide; N-[(3,5-Difluoro-phenyl)-methyl]-4-methyl-2-methylsulfanyl-6-morpholin-4-yl-pyridine-3-carboxylic acid amide; 4-Methyl-2-methylsulfanyl-6-morpholin-4-yl-N-[[4-(trifluoromethyl)-phenyl]-methyl]pyridine-3-carboxylic acid amide; 2-Ethylsulfanyl-N-[(3-fluorophenyl)-methyl]-4-methyl-6-(6-oxo-2,3,4,7,8,8a-hexahydro-1H-pyrrolo[1,2-a]pyrazin-2-yl)-pyridine-3-carboxylic acid amide; 2-Ethylsulfanyl-N-[(3-fluorophenyl)-methyl]-4-methyl-6-(3-oxa-6-azabicyclo[2.2.1]heptan-6-yl)-pyridine-3-carboxylic acid amide; N-(3-Cyano-propyl)-2-ethylsulfanyl-4-methyl-6-morpholin-4-yl-pyridine-3-carboxylic acid amide; 2-Ethylsulfanyl-4-methyl-6-morpholin-4-yl-N-(p-tolyl-methyl)-pyridine-3-carboxylic acid amide; 2-Ethylsulfanyl-4-methyl-N-(3-methylsulfonyl-propyl)-6-morpholin-4-yl-pyridine-3-carboxylic acid amide; N-(4-Cyano-butyl)-2-ethylsulfanyl-4-methyl-6-morpholin-4-yl-pyridine-3-carboxylic acid amide; 2-Ethylsulfanyl-4-methyl-6-morpholin-4-yl-N-(m-tolyl-methyl)-pyridine-3-carboxylic acid amide; N-[(4-Chlorophenyl)-methyl]-2-methoxy-4-methyl-6-morpholin-4-yl-pyridine-3-carboxylic acid amide; N-[(4-Chlorophenyl)-methyl]-2-ethoxy-4-methyl-6-morpholin-4-yl-pyridine-3-carboxylic acid amide; 6-(2-Ethyl-morpholin-4-yl)-2-ethylsulfanyl-N-[(3-fluorophenyl)-methyl]-4-methyl-pyridine-3-carboxylic acid amide; N-[(4-Chlorophenyl)-methyl]-4-methyl-2-methylsulfanyl-6-morpholin-4-yl-pyridine-3-carboxylic acid amide; N-[(4-Fluorophenyl)-methyl]-2-isopropyl-4-methyl-6-[(3R)-3-methyl-morpholin-4-yl]-pyridine-3-carboxylic acid amide; 2-Ethylsulfanyl-N-[(3-fluorophenyl)-methyl]-4-methyl-6-(methyl-pyridin-2-yl-amino)-pyridine-3-carboxylic acid amide; 2-Ethylsulfanyl-N-[(3-fluorophenyl)-methyl]-4-methyl-6-(methyl-pyridin-3-yl-amino)-pyridine-3-carboxylic acid amide; 2-Dimethylamino-N-[(4-fluorophenyl)-methyl]-4-methyl-6-[(3R)-3-methyl-morpholin-4-yl]-pyridine-3-carboxylic acid amide; 2-(Ethyl-methyl-amino)-N-[(4-fluorophenyl)-methyl]-4-methyl-6-[(3R)-3-methyl-morpholin-4-yl]-pyridine-3-carboxylic acid amide; 2-(Ethyl-methyl-amino)-N-[(4-fluorophenyl)-methyl]-4-methyl-6-morpholin-4-yl-pyridine-3-carboxylic acid amide; 2-Ethylsulfanyl-N-[(3-fluorophenyl)-methyl]-4-methyl-6-[methyl-(tetrahydro-pyran-3-yl-methyl)-amino]-pyridine-3-carboxylic acid amide; N-[(4-Fluorophenyl)-methyl]-4-methyl-6-[(3R)-3-methyl-morpholin-4-yl]-2-methylsulfanyl-pyridine-3-carboxylic acid amide; 2-Ethylsulfanyl-N-[(4-fluorophenyl)-methyl]-4-methyl-6-[(3R)-3-methyl-morpholin-4-yl]-pyridine-3-carboxylic acid amide; 6-(3-Ethyl-morpholin-4-yl)-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-6-[(3R)-3-(methoxymethyl)-morpholin-4-yl]-4-methyl-pyridine-3-carboxylic acid amide; 2-Ethylsulfanyl-N-[(3-fluorophenyl)-methyl]-6-[(3S)-3-(methoxymethyl)-morpholin-4-yl]-4-methyl-pyridine-3-carboxylic acid amide; N-[(4-Fluorophenyl)-methyl]-2-methoxy-4-methyl-6-[(3R)-3-methyl-morpholin-4-yl]-pyridine-3-carboxylic acid amide; 2-Ethoxy-N-[(4-fluorophenyl)-methyl]-4-methyl-6-[(3R)-3-methyl-morpholin-4-yl]-pyridine-3-carboxylic acid amide; 2-Dimethylamino-N-(4,4-dimethyl-pentyl)-4-methyl-6-[(3R)-3-methyl-morpholin-4-yl]-pyridine-3-carboxylic acid amide; N-(4,4-Dimethyl-pentyl)-2-(ethyl-methyl-amino)-4-methyl-6-[(3R)-3-methyl-morpholin-4-yl]-pyridine-3-carboxylic acid amide; N-(4,4-Dimethyl-pentyl)-2-isopropyl-4-methyl-6-[(3R)-3-methyl-morpholin-4-yl]-pyridine-3-carboxylic acid amide; N-(4,4-Dimethyl-pentyl)-2-methoxy-4-methyl-6-[(3R)-3-methyl-morpholin-4-yl]-pyridine-3-carboxylic acid amide; N-(4,4-Dimethyl-pentyl)-2-ethoxy-4-methyl-6-[(3R)-3-methyl-morpholin-4-yl]-pyridine-3-carboxylic acid amide; 2-(Ethyl-methyl-amino)-4-methyl-6-morpholin-4-yl-N-[[4-(trifluoromethyl)-phenyl]-methyl]pyridine-3-carboxylic acid amide; N-(4,4-Dimethyl-pentyl)-2-(ethyl-methyl-amino)-4-methyl-6-morpholin-4-yl-pyridine-3-carboxylic acid amide; 2-(Ethyl-methyl-amino)-4-methyl-6-morpholin-4-yl-N-(4,4,4-trifluoro-butyl)-pyridine-3-carboxylic acid amide; N-[(4-Chlorophenyl)-methyl]-2-(ethyl-methyl-amino)-4-methyl-6-morpholin-4-yl-pyridine-3-carboxylic acid amide; N-(4,4-Dimethyl-pentyl)-4-methyl-6-[(3R)-3-methyl-morpholin-4-yl]-2-methylsulfanyl-pyridine-3-carboxylic acid amide; N-(4,4-Dimethyl-pentyl)-2-ethylsulfanyl-4-methyl-6-[(3R)-3-methyl-morpholin-4-yl]-pyridine-3-carboxylic acid amide; N-[(4-Fluorophenyl)-methyl]-4-methyl-6-[(3R)-3-methyl-morpholin-4-yl]-2-(1-methyl-propyl)-pyridine-3-carboxylic acid amide; N-(4,4-Dimethyl-pentyl)-4-methyl-6-[(3R)-3-methyl-morpholin-4-yl]-2-(1-methyl-propyl)-pyridine-3-carboxylic acid amide; 2-Cyclopropyl-N-[(4-fluorophenyl)-methyl]-4-methyl-6-[(3R)-3-methyl-morpholin-4-yl]-pyridine-3-carboxylic acid amide; N-[(4-Fluorophenyl)-methyl]-4-methyl-6-[(3R)-3-methyl-morpholin-4-yl]-2-propyl-pyridine-3-carboxylic acid amide; 2-Cyclopropyl-N-(4,4-dimethyl-pentyl)-4-methyl-6-[(3R)-3-methyl-morpholin-4-yl]-pyridine-3-carboxylic acid amide; N-(4,4-Dimethyl-pentyl)-4-methyl-6-[(3R)-3-methyl-morpholin-4-yl]-2-propyl-pyridine-3-carboxylic acid amide; 2-Ethylsulfanyl-N-[(3-fluorophenyl)-methyl]-4-methyl-6-(methyl-pyridin-4-yl-amino)-pyridine-3-carboxylic acid amide; 2-Ethylsulfanyl-N-[(4-fluoro-3-methyl-phenyl)-methyl]-4-methyl-6-morpholin-4-yl-pyridine-3-carboxylic acid amide; 2-Ethylsulfanyl-N-(2-hydroxy-3-phenyl-propyl)-4-methyl-6-morpholin-4-yl-pyridine-3-carboxylic acid amide; N-[(3,4-Difluoro-phenyl)-methyl]-2-(ethyl-methyl-amino)-4-methyl-6-morpholin-4-yl-pyridine-3-carboxylic acid amide; N-[(3,5-Difluoro-phenyl)-methyl]-2-(ethyl-methyl-amino)-4-methyl-6-morpholin-4-yl-pyridine-3-carboxylic acid amide; 2-Dimethylamino-N-[(4-fluorophenyl)-methyl]-4-methyl-6-morpholin-4-yl-pyridine-3-carboxylic acid amide; N-[(3,4-Difluoro-phenyl)-methyl]-2-dimethylamino-4-methyl-6-morpholin-4-yl-pyridine-3-carboxylic acid amide; N-[(4-Chlorophenyl)-methyl]-2-ethylsulfanyl-4-methyl-6-[(3R)-3-methyl-morpholin-4-yl]-pyridine-3-carboxylic acid amide; N-[(3,5-Dimethyl-phenyl)-methyl]-2-ethylsulfanyl-4-methyl-6-morpholin-4-yl-pyridine-3-carboxylic acid amide; 2-Ethylsulfanyl-N-heptyl-4-methyl-6-morpholin-4-yl-pyridine-3-carboxylic acid amide; 6-Dimethylamino-N-(4,4-dimethyl-pentyl)-2-ethylsulfanyl-4-methyl-pyridine-3-carboxylic acid amide; N-(4,4-Dimethyl-pentyl)-2-ethylsulfanyl-6-(2-methoxy-ethyl-methyl-amino)-4-methyl-pyridine-3-carboxylic acid amide; N-(4,4-Dimethyl-pentyl)-2-ethylsulfanyl-6-(3-methoxy-propyl-methyl-amino)-4-methyl-pyridine-3-carboxylic acid amide; 2-Ethylsulfanyl-N-[(3-fluorophenyl)-methyl]-4-methyl-6-(3-propyl-morpholin-4-yl)-pyridine-3-carboxylic acid amide; N-[(3-Fluorophenyl)-methyl]-2-methoxy-4-methyl-6-[(3R)-3-methyl-morpholin-4-yl]-pyridine-3-carboxylic acid amide; N-[(4-Chlorophenyl)-methyl]-2-methoxy-4-methyl-6-[(3R)-3-methyl-morpholin-4-yl]-pyridine-3-carboxylic acid amide; N-[(3-Fluorophenyl)-methyl]-4-methyl-2-(1-methyl-propyl)-6-morpholin-4-yl-pyridine-3-carboxylic acid amide; 2-Ethylsulfanyl-N-hexyl-4-methyl-6-morpholin-4-yl-pyridine-3-carboxylic acid amide; N-(4,4-Dimethyl-pentyl)-2-ethylsulfanyl-4-methyl-6-(methyl-tetrahydro-furan-3-yl-amino)-pyridine-3-carboxylic acid amide; N-(4,4-Dimethyl-pentyl)-2-ethylsulfanyl-4-methyl-6-(2-methyl-morpholin-4-yl)-pyridine-3-carboxylic acid amide; 2-tert-Butyl-N-(4,4-dimethyl-pentyl)-4-methyl-6-morpholin-4-yl-pyridine-3-carboxylic acid amide; N-(4,4-Dimethyl-pentyl)-4-methyl-2-(1-methyl-propyl)-6-morpholin-4-yl-pyridine-3-carboxylic acid amide; 2-Ethylsulfanyl-N-[(3-fluorophenyl)-methyl]-4-methyl-6-(2-oxa-6-azaspiro[3.4]octan-6-yl)-pyridine-3-carboxylic acid amide; 2-Ethylsulfanyl-N-[(3-fluorophenyl)-methyl]-6-[(2R)-2-(methoxymethyl)-morpholin-4-yl]-4-methyl-pyridine-3-carboxylic acid amide; 2-Ethylsulfanyl-N-[(3-fluorophenyl)-methyl]-6-[(2S)-2-(methoxymethyl)-morpholin-4-yl]-4-methyl-pyridine-3-carboxylic acid amide; N-[(3,4-Difluoro-phenyl)-methyl]-2-ethylsulfanyl-4-methyl-6-[(3R)-3-methyl-morpholin-4-yl]-pyridine-3-carboxylic acid amide; N-[(3,4-Difluoro-phenyl)-methyl]-2-methoxy-4-methyl-6-[(3R)-3-methyl-morpholin-4-yl]-pyridine-3-carboxylic acid amide; 2-Ethylsulfanyl-N-(3-hydroxy-3-phenyl-propyl)-4-methyl-6-morpholin-4-yl-pyridine-3-carboxylic acid amide; 2-Ethylsulfanyl-N-(2-hydroxy-4-methyl-pentyl)-4-methyl-6-morpholin-4-yl-pyridine-3-carboxylic acid amide; 2-Ethylsulfanyl-N-[(3-fluorophenyl)-methyl]-6-[2-(2-methoxy-ethyl)-morpholin-4-yl]-4-methyl-pyridine-3-carboxylic acid amide; 2-Ethylsulfanyl-N-(5-hydroxy-4,4-dimethyl-pentyl)-4-methyl-6-morpholin-4-yl-pyridine-3-carboxylic acid amide; 2-Ethylsulfanyl-4-methyl-N-[(3-methylsulfonyl-phenyl)-methyl]-6-morpholin-4-yl-pyridine-3-carboxylic acid amide; 2-Ethylsulfanyl-N-[(3-fluorophenyl)-methyl]-4-methyl-6-[2-(trifluoromethyl)-5,6,7,8-tetrahydro-[1,6]naphthyridin-6-yl]pyridine-3-carboxylic acid amide; N-[(3,5-Difluoro-phenyl)-methyl]-2-ethylsulfanyl-4-methyl-6-[(3R)-3-methyl-morpholin-4-yl]-pyridine-3-carboxylic acid amide; N-[(3,5-Difluoro-phenyl)-methyl]-2-methoxy-4-methyl-6-[(3R)-3-methyl-morpholin-4-yl]-pyridine-3-carboxylic acid amide; 2-Ethylsulfanyl-4-methyl-6-[(3R)-3-methyl-morpholin-4-yl]-N-(4,4,4-trifluoro-butyl)-pyridine-3-carboxylic acid amide; 2-Methoxy-4-methyl-6-[(3R)-3-methyl-morpholin-4-yl]-N-(4,4,4-trifluoro-butyl)-pyridine-3-carboxylic acid amide; 2-Ethylsulfanyl-4-methyl-6-[(3R)-3-methyl-morpholin-4-yl]-N-[[4-(trifluoromethyl)-phenyl]-methyl]pyridine-3-carboxylic acid amide; 2-Methoxy-4-methyl-6-[(3R)-3-methyl-morpholin-4-yl]-N-[[4-(trifluoromethyl)-phenyl]-methyl]pyridine-3-carboxylic acid amide; 2-Ethylsulfanyl-N-[(3-fluorophenyl)-methyl]-6-[3-(methoxymethyl)-azetidin-1-yl]-4-methyl-pyridine-3-carboxylic acid amide; 6-(2,5-Dimethyl-morpholin-4-yl)-2-ethylsulfanyl-N-[(3-fluorophenyl)-methyl]-4-methyl-pyridine-3-carboxylic acid amide; 2-Dimethylamino-4-methyl-6-morpholin-4-yl-N-[[4-(trifluoromethyl)-phenyl]-methyl]pyridine-3-carboxylic acid amide; N-[(3,5-Difluoro-phenyl)-methyl]-2-dimethylamino-4-methyl-6-morpholin-4-yl-pyridine-3-carboxylic acid amide; 2-Ethylsulfanyl-N-[(3-fluorophenyl)-methyl]-4-methyl-6-[2-(trifluoromethyl)-5,6,7,8-tetrahydro-imidazo[1,2-a]pyrazin-7-yl]-pyridine-3-carboxylic acid amide; N-[(4-Chlorophenyl)-methyl]-2-dimethylamino-4-methyl-6-morpholin-4-yl-pyridine-3-carboxylic acid amide; 2-Dimethylamino-N-(4,4-dimethyl-pentyl)-4-methyl-6-morpholin-4-yl-pyridine-3-carboxylic acid amide; 2-Dimethylamino-4-methyl-6-morpholin-4-yl-N-(4,4,4-trifluoro-butyl)-pyridine-3-carboxylic acid amide; 2-Ethylsulfanyl-4-methyl-N-[(4-methylsulfonyl-phenyl)-methyl]-6-morpholin-4-yl-pyridine-3-carboxylic acid amide; 2-Ethylsulfanyl-N-[(4-fluorophenyl)-methyl]-6-[(3R)-3-(methoxymethyl)-morpholin-4-yl]-4-methyl-pyridine-3-carboxylic acid amide; 2-Ethylsulfanyl-N-[(4-fluorophenyl)-methyl]-6-[(3S)-3-(methoxymethyl)-morpholin-4-yl]-4-methyl-pyridine-3-carboxylic acid amide; 2-tert-Butyl-N-[(3-fluorophenyl)-methyl]-4-methyl-6-morpholin-4-yl-pyridine-3-carboxylic acid amide; 2-Ethylsulfanyl-N-[(3-fluorophenyl)-methyl]-4-methyl-6-[4-(2,2,2-trifluoro-ethyl)-piperazin-1-yl]-pyridine-3-carboxylic acid amide; 6-(2,2-Dimethyl-morpholin-4-yl)-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-4-methyl-6-[methyl-(2-oxo-propyl)-amino]-pyridine-3-carboxylic acid amide; N-[(4-Chlorophenyl)-methyl]-2-ethylsulfanyl-6-[(2R)-2-(methoxymethyl)-morpholin-4-yl]-4-methyl-pyridine-3-carboxylic acid amide; N-[(4-Chlorophenyl)-methyl]-2-ethylsulfanyl-6-[(2S)-2-(methoxymethyl)-morpholin-4-yl]-4-methyl-pyridine-3-carboxylic acid amide; N-[(4-Chlorophenyl)-methyl]-2-ethylsulfanyl-6-[(3R)-3-(methoxymethyl)-morpholin-4-yl]-4-methyl-pyridine-3-carboxylic acid amide; N-[(4-Chlorophenyl)-methyl]-2-ethylsulfanyl-6-[(3S)-3-(methoxymethyl)-morpholin-4-yl]-4-methyl-pyridine-3-carboxylic acid amide; 2-Ethylsulfanyl-N-[(3-fluorophenyl)-methyl]-4-methyl-6-(methyl-tetrahydro-pyran-4-yl-amino)-pyridine-3-carboxylic acid amide; 2-Ethylsulfanyl-N-[(3-fluorophenyl)-methyl]-6-[(4-methoxy-cyclohexyl)-methyl-amino]-4-methyl-pyridine-3-carboxylic acid amide; 2-Ethylsulfanyl-N-[(3-fluorophenyl)-methyl]-4-methyl-6-[2-(trifluoromethyl)-morpholin-4-yl]-pyridine-3-carboxylic acid amide; 2-Ethylsulfanyl-N-[(3-fluorophenyl)-methyl]-4-methyl-6-(methyl-tetrahydro-pyran-3-yl-amino)-pyridine-3-carboxylic acid amide; 6-(3,5-Dimethyl-morpholin-4-yl)-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-6-[(3S)-3-(hydroxymethyl)-morpholin-4-yl]-4-methyl-pyridine-3-carboxylic acid amide; 2-Ethylsulfanyl-N-[(3-fluorophenyl)-methyl]-6-[(3R)-3-(hydroxymethyl)-morpholin-4-yl]-4-methyl-pyridine-3-carboxylic acid amide; N-[(4-Chlorophenyl)-methyl]-2-isopropyl-4-methyl-6-[(3R)-3-methyl-morpholin-4-yl]-pyridine-3-carboxylic acid amide; N-[(4-Chlorophenyl)-methyl]-4-methyl-6-[(3R)-3-methyl-morpholin-4-yl]-2-propyl-pyridine-3-carboxylic acid amide; 2-Ethylsulfanyl-N-(3-hydroxy-4,4-dimethyl-pentyl)-4-methyl-6-morpholin-4-yl-pyridine-3-carboxylic acid amide; N-[(4-Cyano-3-fluoro-phenyl)-methyl]-2-ethylsulfanyl-4-methyl-6-morpholin-4-yl-pyridine-3-carboxylic acid amide; N-[(4-Chlorophenyl)-methyl]-2-(2-fluoro-ethoxy)-4-methyl-6-morpholin-4-yl-pyridine-3-carboxylic acid amide; N-[(4-Chlorophenyl)-methyl]-2-(2,2-difluoro-ethoxy)-4-methyl-6-morpholin-4-yl-pyridine-3-carboxylic acid amide; N-[(4-Chlorophenyl)-methyl]-2-(cyclopropyl-methoxy)-4-methyl-6-morpholin-4-yl-pyridine-3-carboxylic acid amide; 2-(2,2-Difluoro-ethoxy)-N-[(4-fluorophenyl)-methyl]-4-methyl-6-morpholin-4-yl-pyridine-3-carboxylic acid amide; N-[(4-Chlorophenyl)-methyl]-2-ethoxy-4-methyl-6-[(3R)-3-methyl-morpholin-4-yl]-pyridine-3-carboxylic acid amide; N-(4,4-Dimethyl-pentyl)-2-ethylsulfanyl-4-methyl-6-[(2S)-2-methyl-morpholin-4-yl]-pyridine-3-carboxylic acid amide; N-(4,4-Dimethyl-pentyl)-2-ethylsulfanyl-4-methyl-6-[(2R)-2-methyl-morpholin-4-yl]-pyridine-3-carboxylic acid amide; 2-(Cyclopropyl-methoxy)-N-[(4-fluorophenyl)-methyl]-4-methyl-6-morpholin-4-yl-pyridine-3-carboxylic acid amide; N-[(4-Chlorophenyl)-methyl]-2-isopropyl-6-[(3S)-3-(methoxymethyl)-morpholin-4-yl]-4-methyl-pyridine-3-carboxylic acid amide; N-(4,4-Dimethyl-pentyl)-4-methyl-2-(2-methyl-butyl)-6-morpholin-4-yl-pyridine-3-carboxylic acid amide; N-(4,4-Dimethyl-pentyl)-2-(1,1-dimethyl-propyl)-4-methyl-6-morpholin-4-yl-pyridine-3-carboxylic acid amide; N-(4,4-Dimethyl-pentyl)-2-ethylsulfanyl-4-methyl-6-(methyl-tetrahydro-pyran-3-yl-amino)-pyridine-3-carboxylic acid amide; 2-Ethylsulfanyl-4-methyl-6-morpholin-4-yl-N-[(4-nitrophenyl)-methyl]-pyridine-3-carboxylic acid amide; N-[(4-Chlorophenyl)-methyl]-2-cyclopropyl-4-methyl-6-[(3R)-3-methyl-morpholin-4-yl]-pyridine-3-carboxylic acid amide; N-[(4-Chlorophenyl)-methyl]-2-(2-dimethylaminoethyloxy)-4-methyl-6-morpholin-4-yl-pyridine-3-carboxylic acid amide; 2-Ethylsulfanyl-N-[(4-fluoro-3-methoxy-phenyl)-methyl]-4-methyl-6-morpholin-4-yl-pyridine-3-carboxylic acid amide; N-[(4-Chlorophenyl)-methyl]-2-isopropyl-6-[(2S)-2-(methoxymethyl)-morpholin-4-yl]-4-methyl-pyridine-3-carboxylic acid amide; 2-Ethylsulfanyl-N-(3-hydroxy-4-methyl-pentyl)-4-methyl-6-morpholin-4-yl-pyridine-3-carboxylic acid amide; 2-Ethylsulfanyl-N-[(3-fluoro-4-methoxy-phenyl)-methyl]-4-methyl-6-morpholin-4-yl-pyridine-3-carboxylic acid amide; N-[[4-(Difluoro-methoxy)-phenyl]-methyl]-2-ethylsulfanyl-4-methyl-6-morpholin-4-yl-pyridine-3-carboxylic acid amide; N-(1,3-Dihydro-isobenzofuran-5-yl-methyl)-2-ethylsulfanyl-4-methyl-6-morpholin-4-yl-pyridine-3-carboxylic acid amide; N-[(4-Chlorophenyl)-methyl]-2-cyclopropyl-6-[(2S)-2-(methoxymethyl)-morpholin-4-yl]-4-methyl-pyridine-3-carboxylic acid amide; 2-Ethylsulfanyl-N-[(3-fluorophenyl)-methyl]-6-[(2S)-2-(hydroxymethyl)-morpholin-4-yl]-4-methyl-pyridine-3-carboxylic acid amide; 2-Ethylsulfanyl-N-[(3-fluorophenyl)-methyl]-6-[(2R)-2-(hydroxymethyl)-morpholin-4-yl]-4-methyl-pyridine-3-carboxylic acid amide; 6-(Benzyl-methyl-amino)-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-4-methyl-6-[methyl-(tetrahydro-furan-2-yl-methyl)-amino]-pyridine-3-carboxylic acid amide; 2-Ethylsulfanyl-N-[(3-fluorophenyl)-methyl]-4-methyl-6-[(3R)-3-methyl-morpholin-4-yl]-pyridine-3-carboxylic acid amide; 2-Ethylsulfanyl-N-[(3-fluorophenyl)-methyl]-4-methyl-6-[(3S)-3-methyl-morpholin-4-yl]-pyridine-3-carboxylic acid amide; 2-Ethylsulfanyl-N-[(3-fluorophenyl)-methyl]-4-methyl-6-[methyl-[[4-(trifluoromethyl)-phenyl]-methyl]-amino]-pyridine-3-carboxylic acid amide; 6-(1,1-Dioxo-[1,4]thiazinan-4-yl)-2-ethylsulfanyl-N-[(3-fluorophenyl)-methyl]-4-methyl-pyridine-3-carboxylic acid amide; 6-(Azetidin-1-yl)-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-4-methyl-6-(methyl-tetrahydro-furan-3-yl-amino)-pyridine-3-carboxylic acid amide; 2-Ethylsulfanyl-N-[(3-fluorophenyl)-methyl]-4-methyl-6-(N-methyl-anilino)-pyridine-3-carboxylic acid amide; 6-(2,3-Dihydro-1H-isoindol-2-yl)-2-ethylsulfanyl-N-[(3-fluorophenyl)-methyl]-4-methyl-pyridine-3-carboxylic acid amide; 2-Ethylsulfanyl-N-[(3-fluorophenyl)-methyl]-4-methyl-6-(1,2,3,4-tetrahydro-quinolin-1-yl)-pyridine-3-carboxylic acid amide; 6-(2,3-Dihydro-1H-indol-1-yl)-2-ethylsulfanyl-N-[(3-fluorophenyl)-methyl]-4-methyl-pyridine-3-carboxylic acid amide; 2-Ethylsulfanyl-4-methyl-6-morpholin-4-yl-N-(2,4,4-trimethyl-pentyl)-pyridine-3-carboxylic acid amide; 2-Ethylsulfanyl-N-[(3-fluorophenyl)-methyl]-6-[(3-methoxy-cyclohexyl)-methyl-amino]-4-methyl-pyridine-3-carboxylic acid amide; N-(4,4-Difluoro-pentyl)-2-ethylsulfanyl-4-methyl-6-morpholin-4-yl-pyridine-3-carboxylic acid amide; N-[(4-Fluorophenyl)-methyl]-2-isopropyl-4-methyl-6-morpholin-4-yl-pyridine-3-carboxylic acid amide; N-[(3,4-Difluoro-phenyl)-methyl]-2-isopropyl-4-methyl-6-morpholin-4-yl-pyridine-3-carboxylic acid amide; 2-Isopropyl-4-methyl-6-morpholin-4-yl-N-[[4-(trifluoromethyl)-phenyl]-methyl]-pyridine-3-carboxylic acid amide; 2-Ethylsulfanyl-N-[(3-fluorophenyl)-methyl]-4-methyl-6-(3-oxo-morpholin-4-yl)-pyridine-3-carboxylic acid amide; N-(4,4-Dimethyl-pentyl)-4-methyl-6-morpholin-4-yl-2-propyl-pyridine-3-carboxylic acid amide; N-(4,4-Dimethyl-pentyl)-2-isopropyl-4-methyl-6-morpholin-4-yl-pyridine-3-carboxylic acid amide; 2-Isopropyl-4-methyl-6-morpholin-4-yl-N-(4,4,4-trifluoro-butyl)-pyridine-3-carboxylic acid amide; N-[(3,5-Difluoro-phenyl)-methyl]-2-isopropyl-4-methyl-6-morpholin-4-yl-pyridine-3-carboxylic acid amide; N-[(3-Fluorophenyl)-methyl]-4-methyl-6-morpholin-4-yl-2-(oxetan-3-yloxy)-pyridine-3-carboxylic acid amide; 2-Ethylsulfanyl-N-(4-methoxy-4-methyl-pentyl)-4-methyl-6-morpholin-4-yl-pyridine-3-carboxylic acid amide; 2-Ethylsulfanyl-N-(4-fluoro-4-methyl-pentyl)-4-methyl-6-morpholin-4-yl-pyridine-3-carboxylic acid amide; 4-Methyl-6-morpholin-4-yl-2-propyl-N-(4,4,4-trifluoro-butyl)-pyridine-3-carboxylic acid amide; N-[(3,4-Difluoro-phenyl)-methyl]-4-methyl-6-morpholin-4-yl-2-propyl-pyridine-3-carboxylic acid amide; N-[(3,5-Difluoro-phenyl)-methyl]-4-methyl-6-morpholin-4-yl-2-propyl-pyridine-3-carboxylic acid amide; 4-Methyl-6-morpholin-4-yl-2-propyl-N-[[4-(trifluoromethyl)-phenyl]-methyl]-pyridine-3-carboxylic acid amide; N-(4,4-Dimethyl-2-oxo-pentyl)-2-ethylsulfanyl-4-methyl-6-morpholin-4-yl-pyridine-3-carboxylic acid amide; 2-Ethylsulfanyl-N-[(3-fluorophenyl)-methyl]-4-methyl-6-(8-oxa-3-azabicyclo[3.2.1]octan-3-yl)-pyridine-3-carboxylic acid amide; N-[(4-Chlorophenyl)-methyl]-4-methyl-6-morpholin-4-yl-2-propyl-pyridine-3-carboxylic acid amide; N-[(4-Chlorophenyl)-methyl]-2-isopropyl-4-methyl-6-morpholin-4-yl-pyridine-3-carboxylic acid amide; 2-Cyclopropyl-N-(4,4-dimethyl-pentyl)-4-methyl-6-morpholin-4-yl-pyridine-3-carboxylic acid amide; 2-Cyclopropyl-4-methyl-6-morpholin-4-yl-N-(4,4,4-trifluoro-butyl)-pyridine-3-carboxylic acid amide; 2-Cyclopropyl-N-[(3-fluorophenyl)-methyl]-4-methyl-6-morpholin-4-yl-pyridine-3-carboxylic acid amide; 2-Cyclopropyl-N-[(4-fluorophenyl)-methyl]-4-methyl-6-morpholin-4-yl-pyridine-3-carboxylic acid amide; 2-Cyclopropyl-N-[(3,4-difluoro-phenyl)-methyl]-4-methyl-6-morpholin-4-yl-pyridine-3-carboxylic acid amide; 2-Cyclopropyl-N-[(3,5-difluoro-phenyl)-methyl]-4-methyl-6-morpholin-4-yl-pyridine-3-carboxylic acid amide; 2-Cyclopropyl-4-methyl-6-morpholin-4-yl-N-[[4-(trifluoromethyl)-phenyl]methyl]-pyridine-3-carboxylic acid amide; N-[(4-Chlorophenyl)-methyl]-2-cyclopropyl-4-methyl-6-morpholin-4-yl-pyridine-3-carboxylic acid amide; 2-Ethylsulfanyl-N-[(3-fluorophenyl)-methyl]-4-methoxy-6-morpholin-4-yl-pyridine-3-carboxylic acid amide; N-(4,4-Dimethyl-pentyl)-2-(2-methoxy-ethylsulfanyl)-4-methyl-6-morpholin-4-yl-pyridine-3-carboxylic acid amide; 2-Ethylsulfanyl-N-[(3-fluorophenyl)-methyl]-6-[(4-fluorophenyl)-methyl-(3-methoxy-propyl)-amino]-4-methyl-pyridine-3-carboxylic acid amide; 2-Ethylsulfanyl-4-methyl-6-morpholin-4-yl-N-(3,4,4-trimethyl-pentyl)-pyridine-3-carboxylic acid amide; 2-Ethylsulfanyl-N-[(3-fluorophenyl)-methyl]-6-[3-(2-methoxy-ethyl)-morpholin-4-yl]-4-methyl-pyridine-3-carboxylic acid amide; 2-(Acetyl-methyl-amino)-N-[(3-fluorophenyl)-methyl]-4-methyl-6-morpholin-4-yl-pyridine-3-carboxylic acid amide; 2-Ethylsulfanyl-N-[(3-fluorophenyl)-methyl]-6-[(4-fluorophenyl)-methyl-(2-methoxy-ethyl)-amino]-4-methyl-pyridine-3-carboxylic acid amide; 2-Ethylsulfanyl-4-methyl-N-[3-(3-methyl-oxetan-3-yl)-propyl]-6-morpholin-4-yl-pyridine-3-carboxylic acid amide; N-(4,4-Dimethyl-pent-2-ynyl)-2-ethylsulfanyl-4-methyl-6-morpholin-4-yl-pyridine-3-carboxylic acid amide; 2-Ethylsulfanyl-N-[(3-fluorophenyl)-methyl]-4-methyl-6-(3-oxa-8-azabicyclo[3.2.1]octan-8-yl)-pyridine-3-carboxylic acid amide; 2-Ethylsulfanyl-N-[(3-fluorophenyl)-methyl]-4-(methoxymethyl)-6-morpholin-4-yl-pyridine-3-carboxylic acid amide; N-[(4-Chlorophenyl)-methyl]-4-methyl-2-(1-methyl-propyl)-6-morpholin-4-yl-pyridine-3-carboxylic acid amide; N-(4,4-Dimethyl-hexyl)-2-ethylsulfanyl-4-methyl-6-morpholin-4-yl-pyridine-3-carboxylic acid amide; N-(4,4-Dimethyl-pentyl)-2-(2-methoxy-ethoxy)-4-methyl-6-morpholin-4-yl-pyridine-3-carboxylic acid amide; 2-Ethylsulfanyl-4-methyl-N-[3-(1-methyl-cyclopropyl)-propyl]-6-morpholin-4-yl-pyridine-3-carboxylic acid amide; 2-Cyclopropyl-N-[[4-fluoro-3-(methoxymethyl)-phenyl]-methyl]-4-methyl-6-morpholin-4-yl-pyridine-3-carboxylic acid amide; 2-Ethylsulfanyl-N-[[4-fluoro-3-(methoxymethyl)-phenyl]methyl]-4-methyl-6-[(3R)-3-methyl-morpholin-4-yl]-pyridine-3-carboxylic acid amide; 2-Ethylsulfanyl-N-[[4-fluoro-3-(hydroxymethyl)-phenyl]methyl]-4-methyl-6-morpholin-4-yl-pyridine-3-carboxylic acid amide; N-(4,4-Dimethyl-pentyl)-2-(3-methoxy-propyl)-4-methyl-6-morpholin-4-yl-pyridine-3-carboxylic acid amide; 2-Cyclopropyl-N-[[3-fluoro-4-(methoxymethyl)-phenyl]-methyl]-4-methyl-6-morpholin-4-yl-pyridine-3-carboxylic acid amide; N-[(3-Fluorophenyl)-methyl]-2-(methoxymethyl)-4-methyl-6-morpholin-4-yl-pyridine-3-carboxylic acid amide; N-[(4-Chlorophenyl)-methyl]-2,4-diisopropyl-6-morpholin-4-yl-pyridine-3-carboxylic acid amide; N-(4,4-Dimethyl-pentyl)-2-(2-methoxy-ethyl)-4-methyl-6-morpholin-4-yl-pyridine-3-carboxylic acid amide; N-[(4-Chlorophenyl)-methyl]-2,4-diethyl-6-morpholin-4-yl-pyridine-3-carboxylic acid amide; and N-(4,4-Dimethyl-pentyl)-4-methyl-6-morpholin-4-yl-2-tetrahydro-pyran-4-yl-pyridine-3-carboxylic acid amide, respectively in the form of free compounds; racemic mixtures; mixtures of the enantiomers, diastereomers, or enantiomers and diastereomers in any mixing ratio, or an individual enantiomer or diastereomer; or in the form of the salts of physiologically acceptable acids or bases.

### Formula 24

In an embodiment, the Kv7 channel activator may be selected from one of the following compounds according to Formula 24. Such compounds are described in U.S. Pat. No. 9,168,259 issued Mar. 8, 2016 and corresponding to U.S. application Ser. No. 13/449,472 filed Apr. 18, 2012; which is incorporated by reference in its entirety herein. In the case of any conflict of terminology in the context of Formula 24, this reference incorporated by reference herein controls.

In an embodiment, the Kv7 channel activator is a compound according to formula 24:

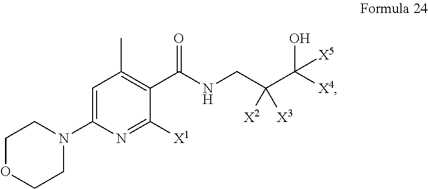

wherein, X¹ represents a C₂₋₆-aliphatic residue, unsubstituted or mono- or polysubstituted; or a C₃₋₆-cycloaliphatic residue or a 3 to 6 membered heterocycloaliphatic residue, in each case unsubstituted or mono- or polysubstituted, and in each case optionally bridged via a C₁₋₄ aliphatic group, which in turn may be unsubstituted or mono- or polysubstituted; or represents OX⁶, wherein X⁶ represents a C₁₋₆-aliphatic residue, unsubstituted or mono- or polysubstituted; or a C₃₋₆-cycloaliphatic residue, unsubstituted or mono- or polysubstituted; X² and X³ independently of one another represent H; F; Cl; Br; I; CN; CH₂F; CHF₂; CF₃; OH; OCH₂F; OCHF₂; OCF₃; SCF₃; a C₁₋₄-aliphatic residue, or an O—C₁₋₄ aliphatic residue, wherein the C₁₋₄ aliphatic residue may in each case be unsubstituted or mono- or polysubstituted; or a C₃₋₆-cycloaliphatic residue or a 3 to 6 membered heterocycloaliphatic residue, in each case unsubstituted or mono- or polysubstituted; with the proviso that at least one of X² and X³ denotes H or is linked via a carbon atom, X⁴ and X⁵ independently of one another represent H; CH₂F; CHF₂; CF₃; a C₁₋₄-aliphatic residue, wherein the C₁₋₄ aliphatic residue may be unsubstituted or mono- or polysubstituted; or a C₃₋₆-cycloaliphatic residue or a 3 to 6 membered heterocycloaliphatic residue, in each case unsubstituted or mono- or polysubstituted; on the condition that if X⁴ and/or X⁵ denote(s) a 3 to 6 membered heterocycloaliphatic residue, the 3 to 6 membered heterocycloaliphatic residue is linked via a carbon atom, or X² and X³ and/or X⁴ and X⁵ in pairs, in each case independently of one another, together with the carbon atom connecting them, form a C₃₋₁₀-cycloaliphatic residue or a 3 to 10 membered heterocycloaliphatic residue, in each case unsubstituted or mono- or polysubstituted, and the respective remaining substituents of X² and X³ each independently of one another represent H; F; Cl; Br; I; CN; CH₂F; CHF₂; CF₃; OH; OCH₂F; OCHF₂; OCF₃; SCF₃; a C₁₋₄-aliphatic residue, or an O—C₁₋₄ aliphatic residue, wherein the C₁₋₄ aliphatic residue may in each case be unsubstituted or mono- or polysubstituted; or a C₃₋₆-cycloaliphatic residue or a 3 to 6 membered heterocycloaliphatic residue, in each case unsubstituted or mono- or polysubstituted, with the proviso that at least one of X² and X³ denotes H or is linked via a carbon atom, and the respective remaining substituents of X⁴ and X⁵ each independently of one another represent H; CH₂F; CHF₂; CF₃; a C₁₋₄-aliphatic residue, wherein the C₁₋₄ aliphatic residue may be unsubstituted or mono- or polysubstituted; or a C₃₋₆-cycloaliphatic residue or a 3 to 6 membered heterocycloaliphatic residue, in each case unsubstituted or mono- or polysubstituted; on the condition that if X⁴ and/or X⁵ denote(s) a 3 to 6 membered heterocycloaliphatic residue, the 3 to 6 membered heterocycloaliphatic residue is linked via a carbon atom, or X² and X⁴ together with the carbon atoms connecting them, form a C₃₋₁₀-cycloaliphatic residue or a 3 to 10 membered heterocycloaliphatic residue, in each case unsubstituted or mono- or polysubstituted, and X³ represents H; F; Cl; Br; I; CN; CH₂F; CHF₂; CF₃; OH; OCH₂F; OCHF₂; OCF₃; SCF₃; a C₁₋₄-aliphatic residue, or an O—C₁₋₄ aliphatic residue, wherein the C₁₋₄ aliphatic residue may in each case be unsubstituted or mono- or polysubstituted; or a C₃₋₆-cycloaliphatic residue or a 3 to 6 membered heterocycloaliphatic residue, in each case unsubstituted or mono- or polysubstituted; and X⁵ represents H; CH₂F; CHF₂; CF₃; a C₁₋₄-aliphatic residue, wherein the C₁₋₄ aliphatic residue may be unsubstituted or mono- or polysubstituted; or a C₃₋₆-cycloaliphatic residue or a 3 to 6 membered heterocycloaliphatic residue, in each case unsubstituted or mono- or polysubstituted; on the condition that if X⁵ denotes a 3 to 6 membered heterocycloaliphatic residue, the 3 to 6 membered heterocycloaliphatic residue is linked via a carbon atom, in which an “aliphatic group” and an “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a N(C₁₋₄-aliphatic residue)-C(═O)—C₁₋₄ aliphatic residue, a NH—S(═O)₂-C₁₋₄ aliphatic residue, a N(C₁₋₄-aliphatic residue)-S(═O)₂-C₁₋₄ aliphatic residue, ═O, OH, OCH₂F, OCHF₂, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)—C₁₋₄-aliphatic residue, a S(═O)₂-C₁₋₄-aliphatic residue, a S(═O)₂-O—C₁₋₄-aliphatic residue, a S(═O)₂—NH—C₁₋₄-aliphatic residue, a S(═O)₂—N(C₁₋₄-aliphatic residue)₂, CN, CH₂F, CHF₂, CF₃, CHO, COOH, a C₁₋₄-aliphatic residue, CH₂OH, CH₂—OCH₃, C₂H₄—OH, C₂H₄—OCH₃ CH₂—CF₃, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH-C(═O)—C₁₋₄ aliphatic residue, a N(C₁₋₄-aliphatic residue)-C(═O)-C₁₋₄ aliphatic residue, a NH—S(═O)₂-C₁₋₄ aliphatic residue, a N(C₁₋₄-aliphatic residue)-S(═O)₂-C₁₋₄ aliphatic residue, ═O, OH, OCH₂F, OCHF₂, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)—C₁₋₄-aliphatic residue, a S(═O)₂-C₁₋₄-aliphatic residue, a S(═O)₂—O—C₁₋₄-aliphatic residue, a S(═O)₂-NH—C₁₋₄-aliphatic residue, a S(═O)₂—N(C₁₋₄-aliphatic residue)₂, CN, CH₂F, CHF₂, CF₃, CHO, COOH, a C₁₋₄-aliphatic residue, CH₂OH, CH₂—OCH₃, C₂H₄—OH, C₂H₄—OCH₃ CH₂—CF₃, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)-N(C₁₋₄ aliphatic residue)₂; said compound being in the form of a free compound, the racemate, a mixture of enantiomers, diastereomers, or of enantiomers and diastereomers in any mixing ratio, or of an individual enantiomer or diastereomer, or in the form of a salt of physiologically acceptable acids or bases.

In further embodiments, X¹ represents a C₂₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represents a C₃₋₆-cycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, and wherein the C₃₋₆-cycloaliphatic residue may optionally be bridged via a C₁₋₄ aliphatic group, which in turn may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represents OX⁶, wherein X⁶ represents a C₁₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue; wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, X² and X³ independently of one another represent H; F; Cl; Br; I; CN; CH₂F; CHF₂; CF₃; OH; OCH₂F; OCHF₂; OCF₃; SCF₃; a C₁₋₄-aliphatic residue, or an O—C₁₋₄ aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, with the proviso that at least one of X² and X³ denotes H or is linked via a carbon atom, X⁴ and X⁵ independently of one another represent H; CH₂F; CHF₂; CF₃; or a C₁₋₄-aliphatic residue, wherein the C₁₋₄-aliphatic residue may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, on the condition that if X⁴ and/or X⁵ denote(s) a 3 to 6 membered heterocycloaliphatic residue, the 3 to 6 membered heterocycloaliphatic residue is linked via a carbon atom, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or X² and X³ and/or X⁴ and X⁵ in pairs, in each case independently of one another, together with the carbon atom connecting them, form a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, and the respective remaining substituents of X² and X³ each independently of one another represent H; F; Cl; Br; I; CN; CH₂F; CHF₂; CF₃; OH; OCH₂F; OCHF₂; OCF₃; SCF₃; a C₁₋₄-aliphatic residue, or an O—C₁₋₄ aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, with the proviso that at least one of X² and X³ denotes H or is linked via a carbon atom, and the respective remaining substituents of X⁴ and X⁵ each independently of one another represent H; CH₂F; CHF₂; CF₃; a C₁₋₄-aliphatic residue, wherein the C₁₋₄-aliphatic residue may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, on the condition that if X⁴ and/or X⁵ denote(s) a 3 to 6 membered heterocycloaliphatic residue, the 3 to 6 membered heterocycloaliphatic residue is linked via a carbon atom, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or X² and X⁴ together with the carbon atoms connecting them, form a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, and the remaining substituent X³ represents H; F; Cl; Br; I; CN; CH₂F; CHF₂; CF₃; OH; OCH₂F; OCHF₂; OCF₃; SCF₃; a C₁₋₄-aliphatic residue, or an O—C₁₋₄ aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represents a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, and the remaining substituent X⁵ represents H; CH₂F; CHF₂; CF₃; OCH₂F; OCHF₂; OCF₃; SCF₃; a C₁₋₄-aliphatic residue, wherein the C₁₋₄-aliphatic residue may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represents a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, on the condition that if X⁵ denotes a 3 to 6 membered heterocycloaliphatic residue, the 3 to 6 membered heterocycloaliphatic residue is linked via a carbon atom, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue.

In further embodiments, X¹ represents a C₂₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represents a C₃₋₆-cycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue, or represents OX⁶, wherein X⁶ represents a C₁₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)-C₁₋₄-aliphatic residue, a S(═O)₂-C₁₋₄-aliphatic residue, CN, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCH₂F, OCHF₂, OCF₃, CH₂F, CHF₂, CF₃ and an unsubstituted O—C₁₋₄-aliphatic residue.

In further embodiments, X¹ represents a C₂₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue is in each case unsubstituted, or represents a C₃₋₆-cycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue is in each case unsubstituted, or represents OX⁶, wherein X⁶ represents a C₁₋₆-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCH₂F, OCHF₂, OCF₃, SH, SCF₃, a S—C₁₋₄-aliphatic residue, a S(═O)—C₁₋₄-aliphatic residue, a S(═O)₂-C₁₋₄-aliphatic residue, CN, and a C₁₋₄-aliphatic residue, wherein the C₁₋₄-aliphatic residue is in each case unsubstituted.

In further embodiments, X¹ represents a C₂₋₆-aliphatic residue, unsubstituted or mono- or polysubstituted with at least one substituent selected from the group consisting of F, Cl, Br, I, OH, and an unsubstituted O—C₁₋₄-aliphatic residue, or represents a C₃₋₆-cycloaliphatic residue, unsubstituted or mono- or polysubstituted with at least one substituent selected from the group consisting of F, Cl, Br, I, OH, and an unsubstituted O—C₁₋₄-aliphatic residue, or represents OX⁶, wherein X⁶ represents a C₁₋₄-aliphatic residue, unsubstituted or mono- or polysubstituted with at least one substituent selected from the group consisting of F, Cl, Br, I, OH, and an unsubstituted O—C₁₋₄-aliphatic residue.

In further embodiments, X¹ represents ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅),

C(CH₃)₂(C₂H₅), ethenyl or propenyl (—CH₂CH═CH₂, —CH═CH—CH₃, —C(═CH₂)—CH₃), cyclopropyl, cyclobutyl, cyclopentyl, or cyclohexyl, or represents OX⁶, wherein X⁶ represents methyl, ethyl, n-propyl, 2-propyl, n-butyl, isobutyl, sec.-butyl, tert.-butyl, CH₂OH, CH₂OCH₃, CH₂CH₂OH, CH₂CH₂OCH₃, and CH(OH) CH₂OH.

In further embodiments, X² and X³ independently of one another represent H; F; Cl; Br; I; CH₂F; CHF₂; CF₃; OH; OCH₂F; OCHF₂; OCF₃; a C₁₋₄-aliphatic residue, or an O—C₁₋₄ aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, with the proviso that at least one of X² and X³ denotes H or is linked via a carbon atom, X⁴ and X⁵ independently of one another represent H; CH₂F; CHF₂; CF₃; a C₁₋₄-aliphatic residue, wherein the C₁₋₄-aliphatic residue may be unsubstituted or mono- or polysubstituted with at least one substituent selected from the group consisting of F, Cl, Br, I, OH, and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, on the condition that if X⁴ and/or X⁵ denote(s) a 3 to 6 membered heterocycloaliphatic residue, the 3 to 6 membered heterocycloaliphatic residue is linked via a carbon atom, or X² and X³ and/or X⁴ and X⁵ in pairs, in each case independently of one another, together with the carbon atom connecting them, form a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, and the respective remaining substituents of X² and X³ each independently of one another represent H; F; Cl; Br; I; CH₂F; CHF₂; CF₃; OH; OCH₂F; OCHF₂; OCF₃; a C₁₋₄-aliphatic residue, or an O—C₁₋₄ aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, with the proviso that at least one of X² and X³ denotes H or is linked via a carbon atom, and the respective remaining substituents of X⁴ and X⁵ each independently of one another represent H; CH₂F; CHF₂; CF₃; a C₁₋₄-aliphatic residue, wherein the C₁₋₄-aliphatic residue may be unsubstituted or mono- or polysubstituted with at least one substituent selected from the group consisting of F, Cl, Br, I, OH, and an unsubstituted O—C₁₋₄-aliphatic residue, or represent a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, on the condition that if X⁴ and/or X⁵ denote(s) a 3 to 6 membered heterocycloaliphatic residue, the 3 to 6 membered heterocycloaliphatic residue is linked via a carbon atom, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, or X² and X⁴ together with the carbon atoms connecting them, form a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, and the remaining substituent X³ represents H; F; Cl; Br; I; CH₂F; CHF₂; CF₃; OH; OCH₂F; OCHF₂; OCF₃; a C₁₋₄-aliphatic residue, or an O—C₁₋₄ aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, or represents a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue, and the remaining substituent X⁵ represents H; CH₂F; CHF₂; CF₃; a C₁₋₄-aliphatic residue, wherein the C₁₋₄-aliphatic residue may be unsubstituted or mono- or polysubstituted with at least one substituent selected from the group consisting of F, Cl, Br, I, OH, and an unsubstituted O—C₁₋₄-aliphatic residue, or represents a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, OCH₂F, OCHF₂, OCF₃, and a C₁₋₄-aliphatic residue, on the condition that if X⁵ denotes a 3 to 6 membered heterocycloaliphatic residue, the 3 to 6 membered heterocycloaliphatic residue is linked via a carbon atom, wherein the C₁₋₄-aliphatic residue in each case may be unsubstituted or mono- or polysubstituted with at least one substituent selected from the group consisting of F, Cl, Br, I, OH, and an unsubstituted O—C₁₋₄-aliphatic residue.

In further embodiments, X² and X³ independently of one another represent H; F; Cl; Br; I; CH₂F; CHF₂; CF₃; OH; a C₁₋₄-aliphatic residue, or an O—C₁₋₄ aliphatic residue, wherein the C₁₋₄-aliphatic residue is in each case unsubstituted, or represent a C₃₋₆-cycloaliphatic residue or a 3 to 6 membered heterocycloaliphatic residue, in each case unsubstituted, with the proviso that at least one of X² and X³ denotes H or is linked via a carbon atom, X⁴ and X⁵ independently of one another represent H; CH₂F; CHF₂; CF₃; a C₁₋₄-aliphatic residue, wherein the C₁₋₄-aliphatic residue is unsubstituted, or represent a C₃₋₆-cycloaliphatic residue or a 3 to 6 membered heterocycloaliphatic residue, in each case unsubstituted, on the condition that if X⁴ and/or X⁵ denote(s) a 3 to 6 membered heterocycloaliphatic residue, the 3 to 6 membered heterocycloaliphatic residue is linked via a carbon atom, or X² and X³ and/or X⁴ and X⁵ in pairs, in each case independently of one another, together with the carbon atom connecting them, form a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, and a C₁₋₄-aliphatic residue, wherein the C₁₋₄-aliphatic residue is in each case unsubstituted, and the respective remaining substituents of X² and X³ each independently of one another represent H; F; Cl; Br; I; CH₂F; CHF₂; CF₃; OH; a C₁₋₄-aliphatic residue, or an O—C₁₋₄ aliphatic residue, wherein the C₁₋₄-aliphatic residue is in each unsubstituted, or represent a C₃₋₆-cycloaliphatic residue or a 3 to 6 membered heterocycloaliphatic residue, in each case unsubstituted, with the proviso that at least one of X² and X³ denotes H or is linked via a carbon atom, and the respective remaining substituents of X⁴ and X⁵ each independently of one another represent H; CH₂F; CHF₂; CF₃; a C₁₋₄-aliphatic residue, wherein the C₁₋₄-aliphatic residue is unsubstituted, or represent a C₃₋₆-cycloaliphatic residue or a 3 to 6 membered heterocycloaliphatic residue, in each case unsubstituted, on the condition that if X⁴ and/or X⁵ denote(s) a 3 to 6 membered heterocycloaliphatic residue, the 3 to 6 membered heterocycloaliphatic residue is linked via a carbon atom, or X² and X⁴ together with the carbon atoms connecting them, form a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, an O—C₁₋₄-aliphatic residue, CH₂F, CHF₂, CF₃, and a C₁₋₄-aliphatic residue, wherein the C₁₋₄-aliphatic residue is in each case unsubstituted, and the remaining substituent X³ represents H; F; Cl; Br; I; CH₂F; CHF₂; CF₃; OH; a C₁₋₄-aliphatic residue, or an O—C₁₋₄ aliphatic residue, wherein the C₁₋₄-aliphatic residue is in each case unsubstituted, or represents a C₃₋₆-cycloaliphatic residue or a 3 to 6 membered heterocycloaliphatic residue, in each case unsubstituted, and the remaining substituent X⁵ represents H; CH₂F; CHF₂; CF₃; a C₁₋₄-aliphatic residue, wherein the C₁₋₄-aliphatic residue is unsubstituted, or represents a C₃₋₆-cycloaliphatic residue or a 3 to 6 membered heterocycloaliphatic residue, in each case unsubstituted, on the condition that if X⁵ denotes a 3 to 6 membered heterocycloaliphatic residue, the 3 to 6 membered heterocycloaliphatic residue is linked via a carbon atom.

In further embodiments, X² and X³ independently of one another represent H; OH; an unsubstituted C₁₋₄-aliphatic residue, or an unsubstituted O—C₁₋₄ aliphatic residue, with the proviso that at least one of X² and X³ denotes H or is linked via a carbon atom, X⁴ and X⁵ independently of one another represent H; CH₂F; CHF₂; CF₃; an unsubstituted C₁₋₄-aliphatic residue, or X² and X³ or X⁴ and X⁵ in pairs, in each case independently of one another, together with the carbon atom connecting them, form a C₃₋₁₀-cycloaliphatic residue, unsubstituted or mono- or polysubstituted with at least one substituent selected from the group consisting of F, Cl, Br, I, OH, CH₂F, CHF₂, CF₃, an unsubstituted O—C₁₋₄-aliphatic residue, and an unsubstituted C₁₋₄-aliphatic residue, and the respective remaining substituents of X² and X³ each independently of one another represent H; OH; an unsubstituted C₁₋₄-aliphatic residue, or an unsubstituted O—C₁₋₄ aliphatic residue, with the proviso that at least one of X² and X³ denotes H or is linked via a carbon atom, and the respective remaining substituents of X⁴ and X⁵ each independently of one another represent H; CH₂F; CHF₂; CF₃; an unsubstituted C₁₋₄-aliphatic residue, or X² and X⁴ together with the carbon atoms connecting them, form a C₃₋₁₀-cycloaliphatic residue, unsubstituted or mono- or polysubstituted with at least one substituent selected from the group consisting of F, Cl, Br, I, OH, an unsubstituted aliphatic residue, CH₂F, CHF₂, CF₃, and an unsubstituted C₁₋₄-aliphatic residue, and the remaining substituent X³ represents H; F; Cl; Br; I; OH; an unsubstituted C₁₋₄-aliphatic residue, or an unsubstituted O—C₁₋₄ aliphatic residue, and the remaining substituent X⁵ represents H; CH₂F; CHF₂; CF₃; an unsubstituted C₁₋₄-aliphatic residue.

In further embodiments, X² and X³ independently of one another represent H; methyl; ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), OCH₃ or OCH₂CH₃, with the proviso that at least one of X² and X³ denotes H or is linked via a carbon atom, X⁴ and X⁵ independently of one another represent H; methyl; ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), CH₂F; CHF₂; CF₃; or X² and X³ or X⁴ and X⁵ in pairs, in each case independently of one another, together with the carbon atom connecting them, form a C₃₋₆-cycloaliphatic residue selected from the group consisting of cyclopropyl, cyclobutyl, cyclopentyl and cyclohexyl, unsubstituted or mono- or polysubstituted with at least one substituent selected from the group consisting of F, Cl, Br, I, OH, methyl; ethyl, n-propyl, 2-propyl, n-butyl, isobutyl, sec.-butyl, tert.-butyl, CH₂F, CHF₂, CF₃, OCH₃ and OCH₂CH₃, and the respective remaining substituents of X² and X³ each independently of one another represents H; methyl; ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), OCH₃ or OCH₂CH₃, with the proviso that at least one of X² and X³ denotes H or is linked via a carbon atom, and the respective remaining substituents of X⁴ and X⁵ each independently of one another represents H; methyl; ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), CH₂F; CHF₂; CF₃; or X² and X⁴ together with the carbon atoms connecting them, form a C₃₋₆-cycloaliphatic residue selected from the group consisting of cyclopropyl, cyclobutyl, cyclopentyl and cyclohexyl, unsubstituted or mono- or polysubstituted with at least one substituent selected from the group consisting of F, Cl, Br, I, OH, methyl; ethyl, n-propyl, 2-propyl, n-butyl, isobutyl, sec.-butyl, tert.-butyl, CH₂F, CHF₂, CF₃, OCH₃ and OCH₂CH₃, and the respective remaining substituent of X³ represents H; methyl; ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), OCH₃ or OCH₂CH₃, and the respective remaining substituent X⁵ represents H; methyl; ethyl, n-propyl, 2-propyl, n-butyl, isobutyl, sec.-butyl, tert.-butyl, n-pentyl, isopentyl, neopentyl, n-hexyl, CH₂—CH(CH₃)(C₂H₅), C(CH₃)₂(C₂H₅), CH₂F; CHF₂; CF₃.

In further embodiments, X¹ represents ethyl, n-propyl, 2-propyl, n-butyl, isobutyl, sec.-butyl, tert.-butyl, cyclopropyl, cyclobutyl, cyclopentyl, or cyclohexyl, or represents OX⁶, wherein X⁶ represents methyl, ethyl, n-propyl, 2-propyl, n-butyl, isobutyl, sec.-butyl, or tert.-butyl, X² and X³ in dependently of one another represent H; methyl; ethyl, n-propyl, 2-propyl, n-butyl, isobutyl, sec.-butyl, or tert.-butyl, X⁴ and X⁵ independently of one another represent H; methyl; ethyl, n-propyl, 2-propyl, n-butyl, isobutyl, sec.-butyl, tert.-butyl, CH₂F, CHF₂, or CF₃; or X² and X³ or X⁴ and X⁵ in pairs, in each case independently of one another, together with the carbon atom connecting them, form a C₃₋₆-cycloaliphatic residue selected from the group consisting of cyclopropyl, cyclobutyl, cyclopentyl and cyclohexyl, unsubstituted or mono- or polysubstituted with at least one substituent selected from the group consisting of OH, methyl; ethyl, n-propyl, 2-propyl, n-butyl, isobutyl, sec.-butyl, CH₂F, CHF₂, CF₃, OCH₃ and OCH₂CH₃, and the respective remaining substituents of X² and X³ in dependently of one another represent H; methyl; ethyl, n-propyl, 2-propyl, n-butyl, isobutyl, sec.-butyl, or tert.-butyl, and the respective remaining substituents of X⁴ and X⁵ independently of one another represent H; methyl; ethyl, n-propyl, 2-propyl, n-butyl, isobutyl, sec.-butyl, tert.-butyl, CH₂F, CHF₂, or CF₃; or X² and X⁴ together with the carbon atoms connecting them, form a C₃₋₆-cycloaliphatic residue selected from the group consisting of cyclopropyl, cyclobutyl, cyclopentyl and cyclohexyl, unsubstituted or mono- or polysubstituted with at least one substituent selected from the group consisting of OH, methyl; ethyl, n-propyl, 2-propyl, n-butyl, isobutyl, sec.-butyl, tert.-butyl, CH₂F, CHF₂, CF₃, OCH₃ and OCH₂CH₃, and X³ represents H; methyl; ethyl, n-propyl, 2-propyl, n-butyl, isobutyl, sec.-butyl, or tert.-butyl, and X⁵ represents H; methyl; ethyl, n-propyl, 2-propyl, n-butyl, isobutyl, sec.-butyl, tert.-butyl, CH₂F, CHF₂, or CF₃.

In further embodiments, the Kv7 cannel activator is selected from the group consisting of: 2-Cyclopropyl-N-(3-hydroxy-4,4-dimethyl-pentyl)-4-methyl-6-morpholin-4-yl-pyridine-3-carboxylic acid amide; 2-Cyclopropyl-N-[[(1S,2R)-2-hydroxy-cyclohexyl]-methyl]-4-methyl-6-morpholin-4-yl-pyridine-3-carboxylic acid amide; 2-Cyclopropyl-N-[[(1R,2S)-2-hydroxy-cyclohexyl]-methyl]-4-methyl-6-morpholin-4-yl-pyridine-3-carboxylic acid amide; 2-Cyclopropyl-N-([2-hydroxy-cyclopentyl]-methyl)-4-methyl-6-morpholin-4-yl-pyridine-3-carboxylic acid amide; N-(3-Hydroxy-4,4-dimethyl-pentyl)-2-isopropyl-4-methyl-6-morpholin-4-yl-pyridine-3-carboxylic acid amide; 2-Isopropyl-4-methyl-6-morpholin-4-yl-N-(4,4,4-trifluoro-3-hydroxy-butyl)-pyridine-3-carboxylic acid amide; and N-[[(1S,2R)-2-Hydroxy-cyclohexyl]-methyl]-2-isopropyl-4-methyl-6-morpholin-4-yl-pyridine-3-carboxylic acid amide; optionally in the form of a single stereoisomer or a mixture of stereoisomers, in the form of the free compound and/or a physiologically acceptable salt thereof.

### Formula 25

In an embodiment, the Kv7 channel activator may be selected from one of the following compounds according to Formula 25. Such compounds are described in U.S. Pat. No. 8,653,101 issued Feb. 18, 2014 and corresponding to U.S. application Ser. No. 10/992,118 filed Nov. 18, 2005; International Publication No. WO2012025236A1, published Mar. 1, 2012 and corresponding to International Application No. PCT/EP2011/004277 filed Aug. 26, 2011; which are incorporated by reference in their entirety herein. In the case of any conflict of terminology in the context of Formula 25, these references incorporated by reference herein control.

In an embodiment, the Kv7 channel activator is a compound according to formula 25.

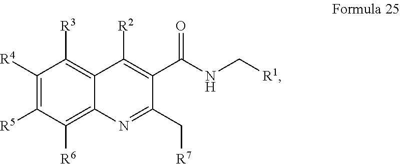

wherein, R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents H; F; Cl; Br; I; CN; CF₃; C(═O) H; NO₂; OCF₃; SCF₃; a C₁₋₄-aliphatic residue, a C(═O)C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₈-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³, R⁴, R⁵ and R⁶ each independently of one another represent H; F; Cl; Br; I; CN; CF₃; C(═O) H; C(═O)—OH; C(═O)—NH₂; SCF₃; S(═O)₂—OH; NO₂; OCF₃; a C₁₋₄-aliphatic residue, a C(═O)-C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(O)—C₁₋₄ aliphatic residue, a NH—S(═O)₂-C₁₋₄-aliphatic residue, a N(C₁₋₄ aliphatic residue)-C(═O)-C₁₋₄ aliphatic residue, or a N(C₁₋₄ aliphatic residue)-S(═O)₂-C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷ denotes a 3 to 10 membered heterocycloaliphatic residue, the 3 to 10 membered heterocycloaliphatic residue is linked via a carbon atom, in which an “aliphatic group” and “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, —0, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)-C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)-C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

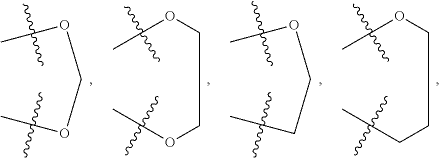 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂-O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂; in the form of the free compounds, the racemate, the enantiomers, diastereomers, mixtures of the enantiomers or diastereomers in any mixing ratio, or of an individual enantiomer or diastereomer, or in the form of the salts of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an

NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

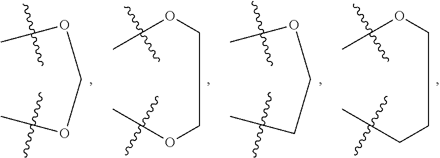 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)-C₂H₅, C(═O)O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH, R² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁-4 aliphatic residue, a C(═O)-N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂-C₁₋₄-aliphatic residue, a NH(C₁₋₄ aliphatic residue), a N(C₁₋₄ aliphatic residue)₂, a NH—C(—O)-C₁₋₄ aliphatic residue, and a NH—S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, R⁷ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁-4 aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁷ denotes a 3 to 10 membered heterocycloaliphatic residue, the 3 to 10 membered heterocycloaliphatic residue is linked via a carbon atom.

In further embodiments, R² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted aliphatic residue, a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, ═O, OH, an unsubstituted C₁₋₄-aliphatic residue and an unsubstituted O—C₁₋₄-aliphatic residue.

In further embodiments, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)—C₁₋₄ aliphatic residue, a C(—O)-O—C₁₋₄ aliphatic residue, a O—C₁₋₄-aliphatic residue, a O—C(═O)—C₁₋₄-aliphatic residue, a S-C₁₋₄-aliphatic residue, a S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, —O, OH, a C₁₋₄-aliphatic residue and a O—C₁₋₄-aliphatic residue, and in each case optionally bridged via an unsubstituted C₁₋₄ aliphatic group.

In further embodiments, R³, R⁴, R⁵ and R⁶ each independently of one another are selected from the group consisting of H; F; Cl; Br; I; NO₂; CF₃; CN; OCF₃; SCF₃; a (C═O)-C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃.

In further embodiments, at least one of R³, R⁴, R⁵ and R⁶ is ≠H.

In further embodiments, R¹ represents the partial structure:

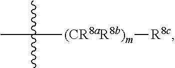

wherein: m denotes 0, 1, 2, 3 or 4, R^{8a} and R^{8b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, R^{8c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

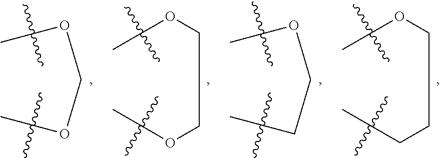 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

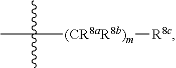

wherein: m denotes 0, 1, or 2, R^{8a} and R^{8b} each independently of one another represent H, F, Cl, Br, I, an O—C₁₋₄ aliphatic residue or a C₁₋₄ aliphatic residue, R^{8c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(—O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In further embodiments, R⁷ denotes a C₁₋₁₀-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue. on the condition that if R⁷ denotes a 3 to 10 membered heterocycloaliphatic residue, the 3 to 10 membered heterocycloaliphatic residue is linked via a carbon atom.

In further embodiments, R⁷ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted C₁₋₄-aliphatic residue, and wherein the C₃₋₁₀-cycloaliphatic residue or the 3 to 10 membered heterocycloaliphatic residue is bridged via a unsubstituted C₁₋₈ aliphatic group.

In further embodiments, R¹ represents the partial structure:

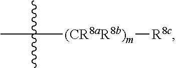

wherein: m is 0, 1 or 2 and R^{8a} and R^{8b} each independently of one another represent H, F, a O—C₁₋₄ aliphatic residue or a C₁₋₄ aliphatic residue; R^{8c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0, R^{8a} and R^{8b} each independently of one another represent H, F, a O—C₁₋₄ aliphatic residue or a C₁₋₄ aliphatic residue; and R^{8c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl, wherein phenyl may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, R² is selected from the group consisting of H; F; Cl; CF₃; CH₃; C₂H₅, iso-propyl; cyclopropyl; and O—CH₃; R³, R⁴, R⁵ and R⁶ are each independently of one another selected from the group consisting of H; F; Cl; Br; CF₃; CN; OCF₃ and NO₂; R⁷ denotes a C₁₋₆-aliphatic residue, unsubstituted or mono- or polysubstituted with at least one substituent selected from the group consisting of F, Cl, Br, I, OH, —O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue, wherein the C₁₋₄-aliphatic residue in each case is unsubstituted.

In further embodiments, the Kv7 channel activator is selected from the group consisting of: N-(3,3-dimethyl-butyl)-2-methoxy-4-methyl-7-(trifluoromethyl)-quinoline-3-carboxylic acid amide; 2-ethoxy-4-methyl-N-(thiophene-2-yl-methyl)-7-(trifluoromethyl)-quinoline-3-carboxylic acid amide; 2-ethoxy-N-[(4-fluorophenyl)-methyl]-4-methyl-7-(trifluoromethyl)-quinoline-3-carboxylic acid amide; N-[(3-fluorophenyl)-methyl]-2-methoxy-4-methyl-7-(trifluoromethyl)-quinoline-3-carboxylic acid amide; N-[(4-fluorophenyl)-methyl]-2-methoxy-4-methyl-7-(trifluoromethyl)-quinoline-3-carboxylic acid amide; 2-ethoxy-N-[(3-fluorophenyl)-methyl]-4-methyl-7-(trifluoromethyl)-quinoline-3-carboxylic acid amide; N-[(3-fluorophenyl)-methyl]-2-(2-methoxy-ethoxy)-4-methyl-7-(trifluoromethyl)-quinoline-3-carboxylic acid amide; N-[(4-fluorophenyl)-methyl]-2-(2-methoxy-ethoxy)-4-methyl-7-(trifluoromethyl)-quinoline-3-carboxylic acid amide; N-[(3-fluorophenyl)-methyl]-2-(2-hydroxy-ethoxy)-4-methyl-7-(trifluoromethyl)-quinoline-3-carboxylic acid amide; N-[(3-fluorophenyl)-methyl]-2-isopropoxy-4-methyl-7-(trifluoromethyl)-quinoline-3-carboxylic acid amide; N-[(4-fluorophenyl)-methyl]-2-isopropoxy-4-methyl-7-(trifluoromethyl)-quinoline-3-carboxylic acid amide; 2-ethoxy-N-[(3-fluorophenyl)-methyl]-4-methoxy-7-(trifluoremethyl)-quinoline-3-carboxylic acid amide; N-[(3-fluorophenyl)-methyl]-2,4-dimethoxy-7-(trifluoromethyl)-quinoline-3-carboxylic acid amide; 2-ethoxy-N-[(4-fluorophenyl)-methyl]-4-methoxy-7-(trifluoromethyl)-quinoline-3-carboxylic acid amide; 2-ethoxy-6,7-difluoro-N-[(3-fluorophenyl)-methyl]-4-methoxy-quinoline-3-carboxylic acid amide; N-[(4-fluorophenyl)-methyl]-2,4-dimethoxy-7-(trifluoromethyl)-quinoline-3-carboxylic acid amide; 6,7-difluoro-N-[(3-fluorophenyl)-methyl]-2,4-dimethoxy-quinoline-3-carboxylic acid amide; 7-fluoro-N-[(3-fluorophenyl)-methyl]-2-methoxy-4-methyl-quinoline-3-carboxylic acid amide; N-[(3-fluoro-4-methyl-phenyl)-methyl]-2-methoxy-4-methyl-7-(trifluoromethyl)-quinoline-3-carboxylic acid amide; 2-ethoxy-N-[(3-fluoro-4-methyl-phenyl)-methyl]-4-methyl-7-(trifluoromethyl)-quinoline-3-carboxylic acid amide; 2-methoxy-4-methyl-N-(m-tolyl-methyl)-7-(trifluoromethyl)-quinoline-3-carboxylic acid amide; 2-ethoxy-4-methyl-N-(m-tolyl-methyl)-7-(trifluoromethyl)-quinoline-3-carboxylic acid amide; N-[(4-fluoro-3-methyl-phenyl)-methyl]-2-methoxy-4-methyl-7-(trifluoromethyl)-quinoline-3-carboxylic acid amide; 2-ethoxy-N-[(4-fluoro-3-methyl-phenyl)-methyl]-4-methyl-7-(trifluoromethyl)-quinoline-3-carboxylic acid amide; 2-methoxy-4-methyl-N-(p-tolyl-methyl)-7-(trifluoromethyl)-quinoline-3-carboxylic acid amide; 2-ethoxy-4-methyl-N-(p-tolyl-methyl)-7-(trifluoromethyl)-quinoline-3-carboxylic acid amide; 2-ethoxy-4-methyl-N-(4-methyl-pentyl)-7-(trifluoromethyl)-quinoline-3-carboxylic acid amide; 2-methoxy-4-methyl-N-(4-methyl-pentyl)-7-(trifluoromethyl)-quinoline-3-carboxylic acid amide; N-(4,4-dimethyl-pentyl)-2-methoxy-4-methyl-7-(trifluoromethyl)-quinoline-3-carboxylic acid amide; N-(4,4-dimethyl-pentyl)-2-ethoxy-4-methyl-7-(trifluoromethyl)-quinoline-3-carboxylic acid amide; 7-bromo-2-ethoxy-N-[(4-fluorophenyl)-methyl]-4-methyl-quinoline-3-carboxylic acid amide; 7-bromo-2-ethoxy-N-[(3-fluorophenyl)-methyl]-4-methyl-quinoline-3-carboxylic acid amide; 7-bromo-N-[(3-fluorophenyl)-methyl]-2-methoxy-4-methyl-quinoline-3-carboxylic acid amide; 7-bromo-N-[(4-fluorophenyl)-methyl]-2-methoxy-4-methyl-quinoline-3-carboxylic acid amide; 7-cyano-2-ethoxy-N-[(4-fluorophenyl)-methyl]-4-methyl-quinoline-3-carboxylic acid amide; 7-cyano-2-ethoxy-N-[(3-fluorophenyl)-methyl]-4-methyl-quinoline-3-carboxylic acid amide; 7-cyano-N-[(3-fluorophenyl)-methyl]-2-methoxy-4-methyl-quinoline-3-carboxylic acid amide; 7-cyano-N-[(4-fluorophenyl)-methyl]-2-methoxy-4-methyl-quinoline-3-carboxylic acid amide; N-[(3-fluoro-2-methoxy-phenyl)-methyl]-2-methoxy-4-methyl-7-(trifluoromethyl)-quinoline-3-carboxylic acid amide; N-[(3-fluoro-5-methoxy-phenyl)-methyl]-2-methoxy-4-methyl-7-(trifluoromethyl)-quinoline-3-carboxylic acid amide; N-[(5-fluoro-2-methoxy-phenyl)-methyl]-2-methoxy-4-methyl-7-(trifluoromethyl)-quinoline-3-carboxylic acid amide; N-[(3-fluoro-2-hydroxy-phenyl)-methyl]-2-methoxy-4-methyl-7-(trifluoromethyl)-quinoline-3-carboxylic acid amide; N-[(3-fluoro-5-hydroxy-phenyl)-methyl]-2-methoxy-4-methyl-7-(trifluoromethyl)-quinoline-3-carboxylic acid amide; N-[(5-fluoro-2-hydroxy-phenyl)-methyl]-2-methoxy-4-methyl-7-(trifluoromethyl)-quinoline-3-carboxylic acid amide; N-[(3-fluoro-4-hydroxy-phenyl)-methyl]-2-methoxy-4-methyl-7-(trifluoromethyl)-quinoline-3-carboxylic acid amide; 7-fluoro-N-[(4-fluorophenyl)-methyl]-2-methoxy-4-methyl-quinoline-3-carboxylic acid amide: 5,7-difluoro-N-[(3-fluorophenyl)-methyl]-2-methoxy-4-methyl-quinoline-3-carboxylic acid amide; 6,7-difluoro-N-[(3-fluorophenyl)-methyl]-2-methoxy-4-methyl-quinoline-3-carboxylic acid amide; 7,8-difluoro-N-[(3-fluorophenyl)-methyl]-2-methoxy-4-methyl-quinoline-3-carboxylic acid amide; N-[(3-fluorophenyl)-methyl]-4-methyl-2-(2,2,2-trifluoro-ethoxy)-7-(trifluoromethyl)-quinoline-3-carboxylic acid amide; N-[(3-fluorophenyl)-methyl]-2-methoxy-4-(trifluoromethyl)-quinoline-3-carboxylic acid amide; 2-ethoxy-N-[(3-fluorophenyl)-methyl]-4-(trifluoromethyl)-quinoline-3-carboxylic acid amide; and N-[(3-fluorophenyl)-methyl]-2-isopropoxy-4-(trifluoromethyl)-quinoline-3-carboxylic acid amide; respectively in the form of the free compounds; the racemate; the enantiomers, diastereomers, mixtures of the enantiomers or diastereomers in any mixing ratio or of an individual enantiomer or diastereomer; or in the form of the salts of physiologically acceptable acids or bases.

### Formula 26

In an embodiment, the Kv7 channel activator may be selected from one of the following compounds according to Formula 26. Such compounds are described in International Publication No. WO2012025236A1, published Mar. 1, 2012 and corresponding to International Application No. PCT/EP2011/004277 filed Aug. 26, 2011; U.S. Pat. No. 9,073,862 issued Jul. 7, 2015 and corresponding to U.S. application Ser. No. 14/098,842 filed Nov. 26, 2013; which are incorporated by reference in their entirety herein. In the case of any conflict of terminology in the context of Formula 26, these references incorporated by reference herein control.

In an embodiment, the Kv7 channel activator is a compound according to formula 26:

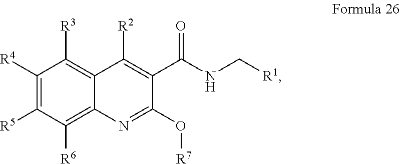

wherein, R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents H; F; Cl; Br; I; CN; CF₃; C(═O) H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³, R⁴, R⁵ and R⁶ each independently of one another represent H; F; Cl; Br; I; CN; CF₃; C(═O) H; C(═O)—OH; C(═O)—NH₂; SCF₃; S(═O)₂—OH; NO₂; OCF₃; a C₁₋₄-aliphatic residue, a C(—O)-C₁₋₄ aliphatic residue, a C(—O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(O)—C₁₋₄ aliphatic residue, a NH—S(═O)₂-C₁₋₄-aliphatic residue, a N(C₁₋₄ aliphatic residue)-C(═O)—C₁₋₄ aliphatic residue, or a N(C₁₋₄ aliphatic residue)-S(═O)₂-C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷ represents an unsubstituted aliphatic residue, then R⁷ does not represent methyl or ethyl, and on the condition that if R⁷ denotes a 3 to 10 membered heterocycloaliphatic residue, the 3 to 10 membered heterocycloaliphatic residue is linked via a carbon atom, in which an “aliphatic group” and “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)—N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)-C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO,

COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)-N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from

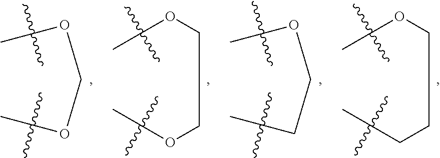 [SMILES: *OCO* ; *OCCO* ; *CCO* ; *CCCO*]

the group consisting of F, Cl, Br, I, NO₂, NH₂, an NH(C₁₋₄ aliphatic residue), an N(C₁₋₄ aliphatic residue)₂, an NH—C(O)—C₁₋₄ aliphatic residue, an NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(—O)—NH(C₁₋₄ aliphatic residue), and a C(═O)—N(C₁₋₄ aliphatic residue)₂; in the form of the free compounds, the racemate, the enantiomers, diastereomers, mixtures of the enantiomers or diastereomers in any mixing ratio, or of an individual enantiomer or diastereomer, or in the form of the salts of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)O—C₂H₅, a C₃₋₆ cycloaliphatic

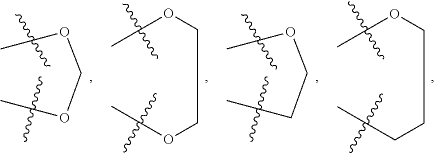 [SMILES: *OCO* ; *OCCO* ; *CCO* ; *CCCO*]

residue, a 3 to 6 membered heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN and C(═O)—OH, R² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, R³, R⁴, R⁵ and R⁶ each independently of one another represent H; F; Cl; Br; I; CN; CF₃; OCF₃; SCF₃; C(═O) H; C(═O)—OH; C(—O)—NH₂; S(═O)₂—OH; NO₂; a C₁₋₄-aliphatic residue, a C(═O)—C₁₋₄ aliphatic residue, a C(—O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S-C₁₋₄-aliphatic residue, a S(═O)₂-C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, R⁷ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(—O)-OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁷ represents an unsubstituted aliphatic residue, then R⁷ does not represent methyl or ethyl, and on the condition that if R⁷ denotes a 3 to 10 membered heterocycloaliphatic residue, the 3 to 10 membered heterocycloaliphatic residue is linked via a carbon atom.

In further embodiments, R² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted O—C₁₋₄-aliphatic residue, a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, —O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue.

In further embodiments, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)-C₁₋₄ aliphatic residue, a C(═O)—O—C₁₋₄ aliphatic residue, a O—C₁₋₄-aliphatic residue, a O—C(═O)—C₁₋₄-aliphatic residue, a S C₁₋₄-aliphatic residue, a S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, —O, OH, a C₁₋₄-aliphatic residue and a O—C₁₋₄-aliphatic residue, and in each case optionally bridged via an unsubstituted C₁₋₄ aliphatic group.

In further embodiments, R³, R⁴, R⁵ and R⁶ each independently of one another are selected from the group consisting of H; F; Cl; Br; I; NO₂; CF₃; CN; OCF₃; SCF₃; a (C═O)-C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃.

In further embodiments, at least one of R³, R⁴, R⁵ and R⁶ is ≠H.

In further embodiments, R¹ represents the partial structure:

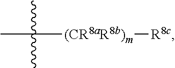

wherein: m denotes 0, 1, 2, 3 or 4, R^{8a} and R^{8b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, R^{8c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

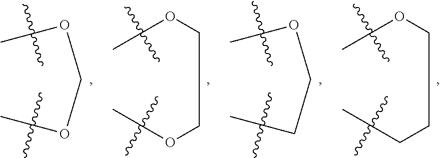 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

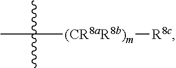

wherein: m denotes 0, 1, or 2, R^{8a} and R^{8b} each independently of one another represent H, F, Cl, Br, I, an O—C₁₋₄ aliphatic residue or a C₁₋₄ aliphatic residue, R^{8c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In further embodiments, R⁷ denotes a C₁₋₁₀-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue, on the condition that if R⁷ represents an unsubstituted aliphatic residue, then R⁷ does not represent methyl or ethyl, and on the condition that if R⁷ denotes a 3 to 10 membered heterocycloaliphatic residue, the 3 to 10 membered heterocycloaliphatic residue is linked via a carbon atom.

In further embodiments, R⁷ denotes a C₁₋₈-aliphatic residue, unsubstituted or mono- or polysubstituted with at least one substituent selected from the group consisting of F, Cl, Br, I, OH, ═O, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue is bridged via a unsubstituted C₁₋₈ aliphatic group, on the condition that if R⁷ represents an unsubstituted aliphatic residue, then R⁷ does not represent methyl or ethyl.

In further embodiments, R¹ represents the partial structure:

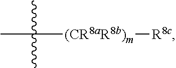

wherein: m is 0, 1 or 2 and R^{8a} and R^{8b} each independently of one another represent H, F, a O—C₁₋₄ aliphatic residue or a C₁₋₄ aliphatic residue; R^{8c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0, R^{8a} and R^{8b} each independently of one another represent H, F, a O—C₁₋₄ aliphatic residue or a C₁₋₄ aliphatic residue; and R^{8c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl, wherein phenyl may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, R² is selected from the group consisting of H; F; Cl; CF₃; CH₃; C₂H₅, iso-propyl; cyclopropyl; and O—CH₃; R³, R⁴, R⁵ and R⁶ are each independently of one another selected from the group consisting of H; F; Cl; Br; CF₃; CN; OCF₃ and NO₂; R⁷ denotes a C₁₋₆-aliphatic residue, unsubstituted or mono- or polysubstituted with at least one substituent selected from the group consisting of F, Cl, Br, I, OH, ═O, an O—C₁₋₄-aliphatic residue, a C(—O)—O—C₁₋₄-aliphatic residue, OCF₃, SH, SCF₃, a S—C₁₋₄-aliphatic residue, CF₃, and a C₁₋₄-aliphatic residue, wherein the C₁₋₄-aliphatic residue in each case is unsubstituted, and on the condition that if R⁷ represents an unsubstituted aliphatic residue, then R⁷ does not represent methyl or ethyl.

In further embodiments, the Kv7 channel activator may be selected from the group consisting of:

N-[(3-fluorophenyl)-methyl]-2-(2-methoxy-ethoxy)-4-methyl-7-(trifluoromethyl)-quinoline-3-carboxylic acid amide; N-[(4-fluorophenyl)-methyl]-2-(2-methoxy-ethoxy)-4-methyl-7-(trifluoromethyl)-quinoline-3-carboxylic acid amide; N-[(3-fluorophenyl)-methyl]-2-(2-hydroxy-ethoxy)-4-methyl-7-(trifluoromethyl)-quinoline-3-carboxylic acid amide; N-[(3-fluorophenyl)-methyl]-2-isopropoxy-4-methyl-7-(trifluoromethyl)-quinoline-3-carboxylic acid amide; N-[(4-fluorophenyl)-methyl]-2-isopropoxy-4-methyl-7-(trifluoromethyl)-quinoline-3-carboxylic acid amide; N-[(3-fluorophenyl)-methyl]-4-methyl-2-(2,2,2-trifluoro-ethoxy)-7-(trifluoromethyl)-quinoline-3-carboxylic acid amide; and N-[(3-fluorophenyl)-methyl]-2-isopropoxy-4-(trifluoromethyl)-quinoline-3-carboxylic acid amide; respectively in the form of the free compounds; the racemate; the enantiomers, diastereomers, mixtures of the enantiomers or diastereomers in any mixing ratio or of an individual enantiomer or diastereomer; or in the form of the salts of physiologically acceptable acids or bases.

### Formula 27

In an embodiment, the Kv7 channel activator may be selected from one of the following compounds according to Formula 27. Such compounds are described in International Publication No. WO2012025238A1, published Mar. 1, 2012 and corresponding to International Application No. PCT/EP2011/004279 filed Aug. 26, 2011; U.S. Pat. No. 8,445,512 issued May 21, 2013 and corresponding to U.S. application Ser. No. 13/218,556 filed Aug. 26, 2011; which are incorporated by reference in their entirety herein. In the case of any conflict of terminology in the context of Formula 27, these references incorporated by reference herein control.

In an embodiment, the Kv7 channel activator is a compound according to formula 27:

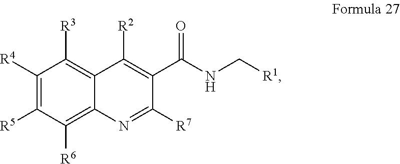

wherein: R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents H; F; Cl; Br; I; CN; CF₃; C(═O) H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₈-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³, R⁴, R⁵ and R⁶ each independently of one another represent H; F; Cl; Br; I; CN; CF₃; C(═O) H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂-C₁₋₄-aliphatic residue, a N(C₁₋₄ aliphatic residue)-C(═O)—C₁₋₄ aliphatic residue, or a N(C₁₋₄ aliphatic residue)-S(═O)₂-C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; on the condition that at least one of R³, R⁴, R⁵ and R⁶ is/H, R⁷ represents a C₂₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷ denotes a 3 to 10 membered heterocycloaliphatic residue, the binding is carried out via a carbon atom of the 3 to 10 membered heterocycloaliphatic residue, in which an “aliphatic group” and “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, —O, OH, OCF₃, a O—C₁₋₄-aliphatic residue, a O—C(O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)-N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)-C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic

C(═O)—O—C₁₋₄-aliphatic residue, a C₃₋₆-cycloaliphatic residue, a 3 to 6 membered heterocycloaliphatic residue, C(—O)—NH₂, a C(═O)—NH(C₁₋₄ aliphatic residue), and a C(═O)—N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

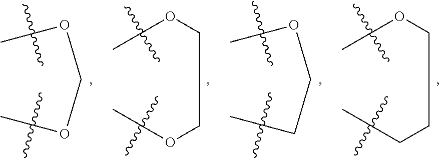 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂; in the form of the free compounds, the racemate, the enantiomers, diastereomers, mixtures of the enantiomers or diastereomers in any mixing ratio, or of an individual enantiomer or diastereomer, or in the form of the salts of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—02H₅, a C₃₋₆ cycloaliphatic residue, a 3 to 6 membered heterocycloaliphatic residue,

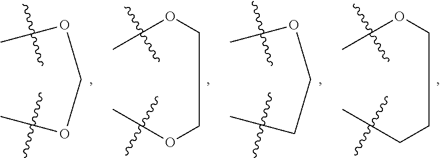 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—O₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN and C(═O)—OH, R² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, R³, R⁴, R⁵ and R⁶ each independently of one another represent H; F; Cl; Br; I; CN; CF₃; OCF₃; SCF₃; C(═O) H; C(—O)-OH; C(—O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂-C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R³, R⁴, R⁵ and R⁶ is ≠H, R⁷ denotes a C₂₋₁₀-aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁷ denotes a 3 to 10 membered heterocycloaliphatic residue, the 3 to 10 membered heterocycloaliphatic residue is linked via a carbon atom.

In further embodiments, R² represents H; F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted aliphatic residue, a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, —O, OH, an unsubstituted C₁₋₄-aliphatic residue and an unsubstituted O—C₁₋₄-aliphatic residue.

In further embodiments, R² is/H.

In further embodiments, R³, R⁴, R⁵ and R⁶ each independently of one another represent H; F; Cl; Br; I; CN; CF₃; OCF₃; SCF₃; C(═O) H; C(═O)—OH; C(═O)—NH₂;

S(═O)₂—OH; NO₂; a C₁₋₄-aliphatic residue, a C(═O)-C₁₋₄ aliphatic residue, a C(═O)—O—C₁₋₄ aliphatic residue, a O—C₁₋₄-aliphatic residue, a O—C(═O)-C₁₋₄-aliphatic residue, a S-C₁₋₄-aliphatic residue, a S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group, on the condition that at least one of R³, R⁴, R⁵ and R⁶ is ≠H.

In further embodiments, R³, R⁴, R⁵ and R⁶ each independently of one another are selected from the group consisting of H; F; Cl; Br; I; NO₂; CF₃; CN; OCF₃; SCF₃; a (C═O)-C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃, on the condition that at least one of R³, R⁴, R⁵ and R⁶ is ≠H.

In further embodiments, R¹ represents the partial structure:

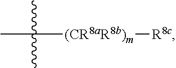

Wherein, m denotes 0, 1, 2, 3 or 4, R^{8a} and R^{8b} each independently of one another represent H, F, Cl, Br, I, NO₂, NH₂, a NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, R^{8c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁-4 aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—O₂H₅, C(═O)—O—CH₃ and C(═O)—O—O₂H₅, a C₃₋₆ cycloaliphatic residue, a 3 to 6 membered heterocycloaliphatic residue,

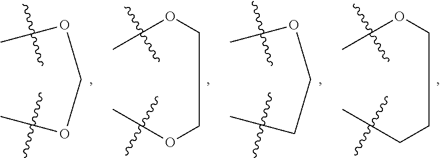 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—O₂H₅, C(═O)—O—CH₃ and C(═O)O—O₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

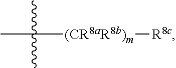

wherein: m denotes 0, 1, or 2, R^{8a} and R^{8b} each independently of one another represent H, F, Cl, Br, I, an O—C₁₋₄ aliphatic residue or a C₁₋₄ aliphatic residue, R^{8c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—O₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—O₂H₅, C(═O)—O—CH₃ and C(═O)—O—O₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In further embodiments, R⁷ denotes a C₂₋₁₀-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue. on the condition that if R⁷ denotes a 3 to 10 membered heterocycloaliphatic residue, the 3 to 10 membered heterocycloaliphatic residue is linked via a carbon atom.

In further embodiments, R⁷ denotes a C₂₋₈-aliphatic residue, unsubstituted or mono- or polysubstituted with at least one substituent selected from the group consisting of F, Cl, Br, I, OH, ═O, an O—C₁₋₄-aliphatic residue, OCF₃, SH, SCF₃, a S—C₁₋₄-aliphatic residue, a C(═O)—O—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue may in each case optionally bridged via a unsubstituted C₁₋₈ aliphatic group, on the condition that if R⁷ denotes a 3 to 10 membered heterocycloaliphatic residue, the 3 to 10 membered heterocycloaliphatic residue is linked via a carbon atom.

In further embodiments, R¹ represents the partial structure:

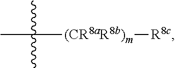

wherein: m is 0, 1 or 2 and R^{8a} and R^{8b} each independently of one another represent H, F, a O—C₁₋₄ aliphatic residue or a C₁₋₄ aliphatic residue; R^{8c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0, R^{8a} and R^{8b} each independently of one another represent H, F, a O—C₁₋₄ aliphatic residue or a C₁₋₄ aliphatic residue; and R^{8c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl, wherein phenyl may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, R² is selected from the group consisting of H; F; Cl; CF₃; CH₃; C₂H₅, iso-propyl; cyclopropyl; and O—CH₃; R³, R⁴, R⁵ and R⁶ are each independently of one another selected from the group consisting of H; F; Cl; Br; CH₃; CF₃; CN; OCF₃ and NO₂; on the condition that at least one of R³, R⁴, R⁵ and R⁶ is ≠H, R⁷ denotes ethyl, n-propyl, 2-propyl, n-butyl, isobutyl, sec.-butyl, tert.-butyl, n-pentyl, isopentyl, neopentyl, n-hexyl, ethenyl or propenyl (—CH₂CH═CH₂, —CH═CH—CH₃, —C(═CH₂)—CH₃), in each case unsubstituted or mono- or polysubstituted with at least one substituent selected from the group consisting of F, Cl, OH, O—CH₃, CF₃, and N(CH₃)₂, or denotes cyclopropyl, cyclobutyl, cyclopentyl and cyclohexyl, piperidinyl tetrahydrofuranyl, or tetrahydropyranyl, in each case unsubstituted or mono- or polysubstituted with at least one substituent selected from the group consisting of F, Cl, OH, an O—C₁₋₄-aliphatic residue, CF₃, and a C₁₋₄-aliphatic residue, wherein the C₁₋₄-aliphatic residue in each case is unsubstituted, and wherein cyclopropyl, cyclobutyl, cyclopentyl and cyclohexyl, piperidinyl tetrahydrofuranyl, or tetrahydropyranyl may in each case be optionally bridged, via an unsubstituted C₁₋₄ aliphatic group, on the condition that if R⁷ denotes piperidinyl tetrahydrofuranyl, or tetrahydropyranyl, each of these residues is linked via a carbon atom.

In further embodiments, the Kv7 channel activator is selected from the group consisting of: 4-methyl-2-propyl-N-(thiophene-2-yl-methyl)-7-(trifluoromethyl)-quinoline-3-carboxylic acid amide; N-(cycloheptyl-methyl)-4-methyl-2-propyl-7-(trifluoromethyl)-quinoline-3-carboxylic acid amide; N-[(4-fluorophenyl)-methyl]-4-methyl-2-propyl-7-(trifluoromethyl)-quinoline-3-carboxylic acid amide; N-[(3-fluorophenyl)-methyl]-4-methyl-2-propyl-7-(trifluoromethyl)-quinoline-3-carboxylic acid amide; 2-ethyl-N-[(4-fluorophenyl)-methyl]-4-methyl-7-(trifluoromethyl)-quinoline-3-carboxylic acid amide; 2-ethyl-N-[(3-fluorophenyl)-methyl]-4-methyl-7-(trifluoromethyl)-quinoline-3-carboxylic acid amide; N-[(4-fluorophenyl)-methyl]-2-isopropyl-4-methyl-7-(trifluoromethyl)-quinoline-3-carboxylic acid amide; N-[(3-fluorophenyl)-methyl]-2-isopropyl-4-methyl-7-(trifluoromethyl)-quinoline-3-carboxylic acid amide; 2-cyclopropyl-N-[(4-fluorophenyl)-methyl]-4-methyl-7-(trifluoromethyl)-quinoline-3-carboxylic acid amide; 2-cyclopropyl-N-[(3-fluorophenyl)-methyl]-4-methyl-7-(trifluoromethyl)-quinoline-3-carboxylic acid amide; N-[(3-fluorophenyl)-methyl]-2-isopropyl-7-(trifluoromethyl)-quinoline-3-carboxylic acid amide; N-[(4-fluorophenyl)-methyl]-2-isopropyl-7-(trifluoromethyl)-quinoline-3-carboxylic acid amide; 2-tert-butyl-N-[(3-fluorophenyl)-methyl]-7-(trifluoromethyl)-quinoline-3-carboxylic acid amide; 2-tert-butyl-N-[(4-fluorophenyl)-methyl]-7-(trifluoromethyl)-quinoline-3-carboxylic acid amide; N-[(3-fluorophenyl)-methyl]-2-(methoxymethyl)-4-methyl-7-(trifluoromethyl)-quinoline-3-carboxylic acid amide; N-[(4-fluorophenyl)-methyl]-2-(methoxymethyl)-4-methyl-7-(trifluoromethyl)-quinoline-3-carboxylic acid amide; N-[(3-fluorophenyl)-methyl]-2-(hydroxymethyl)-4-methyl-7-(trifluoromethyl)-quinoline-3-carboxylic acid amide; 2-(2,2-dimethyl-propyl)-N-[(3-fluorophenyl)-methyl]-4-methyl-7-(trifluoromethyl)-quinoline-3-carboxylic acid amide; 2-(2,2-dimethyl-propyl)-N-[(4-fluorophenyl)-methyl]-4-methyl-7-(trifluoromethyl)-quinoline-3-carboxylic acid amide; 2-cyclopentyl-N-[(3-fluorophenyl)-methyl]-4-methyl-7-(trifluoromethyl)-quinoline-3-carboxylic acid amide; 2-cyclopentyl-N-[(4-fluorophenyl)-methyl]-4-methyl-7-(trifluoromethyl)-quinoline-3-carboxylic acid amide; N-(4,4-dimethyl-pentyl)-4-methyl-2-propyl-7-(trifluoromethyl)-quinoline-3-carboxylic acid amide; N-[(3-fluorophenyl)-methyl]-4-methyl-2-[(E)-prop-1-enyl]-7-(trifluoromethyl)-quinoline-3-carboxylic acid amide; N-[(3-fluorophenyl)-methyl]-4-methyl-2-(2-methyl-prop-1-enyl)-7-(trifluoromethyl)-quinoline-3-carboxylic acid amide; 7-bromo-2-ethyl-N-[(3-fluorophenyl)-methyl]-4-methyl-quinoline-3-carboxylic acid amide; 7-bromo-2-ethyl-N-[(4-fluorophenyl)-methyl]-4-methyl-quinoline-3-carboxylic acid amide; 7-bromo-2-cyclopropyl-N-[(3-fluorophenyl)-methyl]-4-methyl-quinoline-3-carboxylic acid amide; 7-bromo-2-cyclopropyl-N-[(4-fluorophenyl)-methyl]-4-methyl-quinoline-3-carboxylic acid amide; 7-bromo-N-[(3-fluorophenyl)-methyl]-2-isopropyl-4-methyl-quinoline-3-carboxylic acid amide; 7-bromo-N-[(4-fluorophenyl)-methyl]-2-isopropyl-4-methyl-quinoline-3-carboxylic acid amide; 2-(dimethylaminomethyl)-N-[(3-fluorophenyl)-methyl]-4-methyl-7-(trifluoromethyl)-quinoline-3-carboxylic acid amide; 2-ethyl-N-[(4-fluoro-3-methyl-phenyl)-methyl]-4-methyl-7-(trifluoromethyl)-quinoline-3-carboxylic acid amide; 7-cyano-2-ethyl-N-[(3-fluorophenyl)-methyl]-4-methyl-quinoline-3-carboxylic acid amide; 2-ethyl-N-[(3-fluoro-4-methyl-phenyl)-methyl]-4-methyl-7-(trifluoromethyl)-quinoline-3-carboxylic acid amide; 7-cyano-2-ethyl-N-[(4-fluorophenyl)-methyl]-4-methyl-quinoline-3-carboxylic acid amide; N-[(3-fluorophenyl)-methyl]-4-methyl-2-(2-methyl-propyl)-7-(trifluoromethyl)-quinoline-3-carboxylic acid amide; 7-cyano-2-cyclopropyl-N-[(3-fluorophenyl)-methyl]-4-methyl-quinoline-3-carboxylic acid amide; 7-cyano-2-cyclopropyl-N-[(4-fluorophenyl)-methyl]-4-methyl-quinoline-3-carboxylic acid amide; 7-cyano-N-[(3-fluorophenyl)-methyl]-2-isopropyl-4-methyl-quinoline-3-carboxylic acid amide; and 7-cyano-N-[(4-fluorophenyl)-methyl]-2-isopropyl-4-methyl-quinoline-3-carboxylic acid amide; respectively in the form of the free compounds; the racemate; the enantiomers, diastereomers, mixtures of the enantiomers or diastereomers in any mixing ratio or of an individual enantiomer or diastereomer; or in the form of the salts of physiologically acceptable acids or bases.

### Formula 28

In an embodiment, the Kv7 channel activator may be selected from one of the following compounds according to Formula 28. Such compounds are described in International Publication No. WO2012028300A1, published Mar. 8, 2012 and corresponding to International Application No. PCT/EP2011/004369 filed Aug. 31, 2011;

U.S. Pat. No. 8,618,129 issued Dec. 31, 2013 and corresponding to U.S. application Ser. No. 13/222,023 filed Aug. 31, 2011; which are incorporated by reference in their entirety herein. In the case of any conflict of terminology in the context of Formula 28, these references incorporated by reference herein control.

In an embodiment, the Kv7 channel activator is a compound according to formula 28:

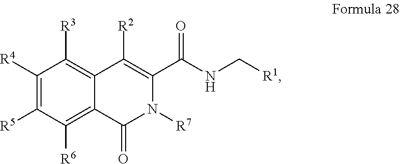

wherein, R¹ represents a C₁₋₁₀-aliphatic residue, unsubstituted or mono- or polysubstituted; a C₃₋₁₀-cycloaliphatic residue, unsubstituted or mono- or polysubstituted and optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents F; Cl; Br; I; CN; CF₃; C(═O) H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a 0-C(═O)-C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³, R⁴, R⁵ and R⁶ each independently of one another represent H; F; Cl; Br; I; CN; CF₃; C(═O) H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)-C₁₋₄ aliphatic residue, a NH—S(═O)₂-C₁₋₄-aliphatic residue, a N(C₁₋₄ aliphatic residue)-C(═O)—C₁₋₄ aliphatic residue, or a N(C₁₋₄ aliphatic residue) S(═O)₂-C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷ denotes a 3 to 10 membered heterocycloaliphatic residue, the 3 to 10 membered heterocycloaliphatic residue is linked via a carbon atom, in which an “aliphatic group” and “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, a NH—C(═O)-C₁₋₄ aliphatic residue, a NH—S(═O)₂-C₁₋₄ aliphatic residue, OH, ═O, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, C(═O)—NH₂, a C(═O)—NH(C₁₋₄ aliphatic residue), and a C(═O)-N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, ═O, OH, OCF₃, a O—C₁₋₄-aliphatic residue, a O—C(═O)-C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic

C(═O)—O—C₁₋₄-aliphatic residue, a C₃₋₆-cycloaliphatic residue, a 3 to 6 membered heterocycloaliphatic residue, C(—O)—NH₂, a C(═O)—NH(C₁₋₄ aliphatic residue), and a C(═O)—N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

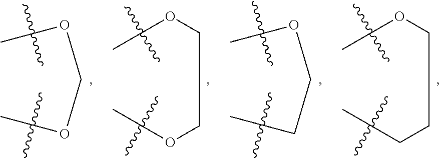 [SMILES: *OCO* ; *OCCO* ; *CCO* ; *CCCO*]

an NH(C₁₋₄ aliphatic residue), an N(C₁₋₄ aliphatic residue)₂, an NH—C(═O)-C₁₋₄ aliphatic residue, an NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂-O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)—C₁₋₄-aliphatic residue, a C(—O)-O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂; in the form of the free compounds, the racemate, the enantiomers, diastereomers, mixtures of the enantiomers or diastereomers in any mixing ratio, or of an individual enantiomer or diastereomer, or in the form of the salts of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, ═O, OH, OCF₃, CF₃ and an unsubstituted O—C₁₋₄-aliphatic residue, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O) OH, and wherein the C₃₋₁₀-cycloaliphatic residue may optionally be bridged via a C₁₋₈ aliphatic group, which in turn may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)-C₂H₅, C(═O)—O—CH₃ and C(═O)—O—C₂H₅, a C₃₋₆ cycloaliphatic residue, a 3 to 6 membered heterocycloaliphatic residue,

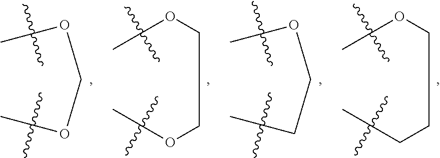 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, ═O, OH, OCF₃, CF₃ and an unsubstituted O—C₁₋₄-aliphatic residue, and wherein benzyl, phenyl, thienyl, pyridyl, furyl, thiazolyl and oxazolyl in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁-4 aliphatic residue, CF₃, CN, a C₁₋₄-aliphatic residue and C(═O)—OH, and wherein the aryl or the heteroaryl residue may in each case be optionally bridged via a C₁₋₈ aliphatic group, which in turn may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN and C(═O)—OH, R² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)-C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂-C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, a C₁₋₄-aliphatic residue and a O—C₁₋₄-aliphatic residue, and in each case optionally bridged via an unsubstituted C₁₋₄ aliphatic group, R⁷ denotes a C₁₋₁₀-aliphatic residue, unsubstituted or mono- or polysubstituted with at least one substituent selected from the group consisting of F, Cl, Br, I, NO₂, OH, ═O, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, ═O, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, —O, OH, OCF₃, CF₃ and an unsubstituted O—C₁₋₄-aliphatic residue, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, ═O, OH, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁷ denotes a 3 to 10 membered heterocycloaliphatic residue, the 3 to 10 membered heterocycloaliphatic residue is linked via a carbon atom.

In further embodiments, R² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, —O, OH, and an unsubstituted O—C₁₋₄-aliphatic residue, a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and an unsubstituted O—C₁₋₄-aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, OH, an unsubstituted C₁₋₄-aliphatic residue and an unsubstituted O—C₁₋₄-aliphatic residue.

In further embodiments, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a O—C₁₋₄-aliphatic residue, a O—C(═O)—C₁₋₄-aliphatic residue, a S-C₁₋₄-aliphatic residue, a S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, ═O, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, —O, OH, a C₁₋₄-aliphatic residue and a O—C₁₋₄-aliphatic residue, and in each case optionally bridged via an unsubstituted C₁₋₄ aliphatic group.

In further embodiments, R³, R⁴, R⁵ and R⁶ each independently of one another are selected from the group consisting of H; F; Cl; Br; I; NO₂; CF₃; CN; OCF₃; SCF₃; a (C═O)-C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃.

In further embodiments, at least one of R³, R⁴, R⁵ and R⁶ is/H.

In further embodiments, R¹ represents the partial structure:

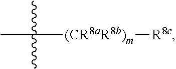

wherein: m denotes 0, 1, 2, 3 or 4, R^{8a} and R^{8b} each independently of one another represent H, F, Cl, Br, I, NO₂, OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, or together denote ═O, R^{8c} denotes a C₁₋₄ aliphatic residue, unsubstituted or mono- or polysubstituted with at least one substituent selected from the group consisting of F, Cl, Br, I, NO₂, ═O, OH, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes a C₃₋₁₀-cycloaliphatic residue, unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I,
OH, OCF₃, CF₃ and an unsubstituted O—C₁₋₄-aliphatic residue, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(—O)-OH, C(═O)—CH₃, C(═O)—C₂H₅, C(═O)—O—CH₃ and C(═O)—O—C₂H₅, a C₃₋₆ cycloaliphatic residue, a 3 to

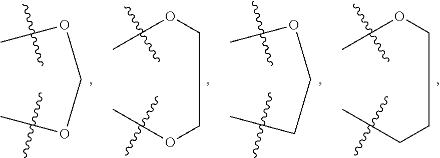 [SMILES: *OCO* ; *OCCO* ; *CCO* ; *CCCO*]

6 membered heterocycloaliphatic residue, benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)O—CH₃ and C(═O)—O—C₂H₅, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure

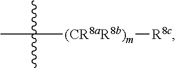

wherein, m denotes 0, 1, or 2, R^{8a} and R^{8b} each independently of one another represent H, F, Cl, Br, I, an O—C₁₋₄ aliphatic residue or a C₁₋₄ aliphatic residue, R^{8c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, in each case may be unsubstituted or mono- or polysubstituted, preferably unsubstituted or mono- or disubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, preferably with at least one substituent selected from the group consisting of F, Cl, CH₃, O—CH₃, CF₃ and OCF₃, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In further embodiments, R⁷ denotes a C₁₋₁₀-aliphatic residue, unsubstituted or mono- or polysubstituted with at least one substituent selected from the group consisting of F, Cl, Br, I, NO₂, ═O, OH, an O—C₁₋₄-aliphatic residue, OCF₃, SH, SCF₃, a S—C₁₋₄-aliphatic residue, a C(═O)—O—C₁₋₄-aliphatic residue, CF₃, CN, and a C₁₋₄-aliphatic residue wherein the C₁₋₄-aliphatic residue in each case may be unsubstituted or mono- or polysubstituted with at least one substituent selected from the group consisting of F, Cl, Br, I, ═O, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, ═O, OH, OCF₃, CF₃ and an unsubstituted O—C₁₋₄-aliphatic residue, and wherein the C₃₋₆ cycloaliphatic residue and the 3 to 6 membered heterocycloaliphatic residue in each case may be unsubstituted or mono- or polysubstituted with at least one substituent selected from the group consisting of F, Cl, Br, I, NO₂, OH, —O, an O—C₁₋₄ aliphatic residue, OCF₃, SH, SCF₃, a S—C₁₋₄ aliphatic residue, CF₃, CN, and a C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, OH, ═O, an O—C₁₋₄ aliphatic residue, a C(—O)—O—C₁₋₄-aliphatic residue, OCF₃, SH, SCF₃, a S—C₁₋₄ aliphatic residue, CF₃, CN, and a C₁₋₄-aliphatic residue. on the condition that if R⁷ denotes a 3 to 10 membered heterocycloaliphatic residue, the 3 to 10 membered heterocycloaliphatic residue is linked via a carbon atom.

In further embodiments, R⁷ denotes a C₁₋₈-aliphatic residue, unsubstituted or mono- or polysubstituted with at least one substituent selected from the group consisting of F, Cl, Br, I, OH, an O—C₁₋₄-aliphatic residue, a C(═O)—O—C₁₋₄-aliphatic residue, OCF₃, SH, SCF₃, a S—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₁₀-cycloaliphatic residue or the 3 to 10 membered heterocycloaliphatic residue in each case may be bridged via an unsubstituted C₁₋₈ aliphatic group, on the condition that if R⁷ denotes a 3 to 10 membered heterocycloaliphatic residue, the 3 to 10 membered heterocycloaliphatic residue is linked via a carbon atom.

In further embodiments, R¹ represents the partial structure:

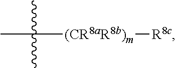

wherein: m is 0, 1 or 2 and R^{8a} and R^{8b} each independently of one another represent H, F, a O—C₁₋₄ aliphatic residue or a C₁₋₄ aliphatic residue; R^{8c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or denotes a C₃₋₁₀-cycloaliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0, R^{8a} and R^{8b} each independently of one another represent H, F, a O—C₁₋₄ aliphatic residue or a C₁₋₄ aliphatic residue; R^{8c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl, wherein phenyl may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, R² is selected from the group consisting of F; Cl; CF₃; CH₃; C₂H₅, iso-propyl; cyclopropyl; and O—CH₃; R³, R⁴, R⁵ and R⁶ are each independently of one another selected from the group consisting of H; F; Cl; CF₃; CN; OCF₃ and NO₂; R⁷ denotes a C₁₋₆-aliphatic residue, unsubstituted or mono- or polysubstituted with at least one substituent selected from the group consisting of F, Cl, Br, I, OH, an O—C₁₋₄-aliphatic residue, a C(═O)—O—C₁₋₄-aliphatic residue, OCF₃, SH, SCF₃, a S—C₁₋₄-aliphatic residue, CF₃, and a

C₁₋₄-aliphatic residue, wherein the C₁₋₄-aliphatic residue in each case may be unsubstituted or mono- or polysubstituted with at least one substituent selected from the group consisting of OH, and an unsubstituted O—C₁₋₄-aliphatic residue.

In further embodiments, the Kv7 channel activator is selected from the group consisting of: N-(3-fluorobenzyl)-2,4-dimethyl-1-oxo-7-(trifluoromethyl)-1,2-dihydroisoquinoline-3-carboxamide; N-(3-fluorobenzyl)-4-methyl-1-oxo-2-propyl-7-(trifluoromethyl)-1,2-dihydroisoquinoline-3-carboxamide; 2-ethyl-N-(3-fluorobenzyl)-4-methyl-1-oxo-7-(trifluoromethyl)-1,2-dihydroisoquinoline-3-carboxamide; N-(3-fluorobenzyl)-2-isopropyl-4-methyl-1-oxo-7-(trifluoromethyl)-1,2 dihydroisoquinoline-3-carboxamide; and N-(3-fluorobenzyl)-2-(2-methoxyethyl)-4-methyl-1-oxo-7-(trifluoromethyl)-1,2-dihydroisoquinoline-3-carboxamide; respectively in the form of the free compounds; the racemate; the enantiomers, diastereomers, mixtures of the enantiomers or diastereomers in any mixing ratio or of an individual enantiomer or diastereomer; or in the form of the salts of physiologically acceptable acids or bases.

### Formula 29

In an embodiment, the Kv7 channel activator may be selected from one of the following compounds according to Formula 29. Such compounds are described in U.S. Pat. No. 8,653,102 issued Feb. 18, 2014 and corresponding to U.S. application Ser. No. 13/218,579 filed Aug. 26, 2011; International Publication No. PCT/EP2011/004280, published Mar. 1, 2012, and corresponding to International Application No. WO2012025239A1 filed Aug. 25, 2011; which are incorporated by reference in their entirety herein. In the case of any conflict of terminology in the context of Formula 29, these references incorporated by reference herein control.

In an embodiment, the Kv7 channel activator is a compound according to formula 29:

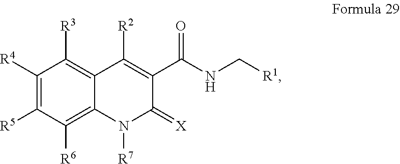

wherein: X denotes O or S, R¹ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted; R² represents F; Cl; Br; I; CN; CF₃; C(═O) H; NO₂; OCF₃; SCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R³, R⁴, R⁵ and R⁶ each independently of one another represent H; F; Cl; Br; I; CN; CF₃; C(═O) H; C(═O)—OH; C(═O)—NH₂; SCF₃; S(═O)₂—OH; NO₂; OCF₃; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)—N(C₁₋₄ aliphatic residue)₂, wherein the C₁₋₄ aliphatic residue may be in each case be unsubstituted or mono- or polysubstituted; a O—C₁₋₄-aliphatic residue, a O—C(═O)—C₁₋₄-aliphatic residue, a S—C₁₋₄-aliphatic residue, a S(═O)₂-C₁₋₄-aliphatic residue, a S(═O)₂—O—C₁₋₄-aliphatic residue, wherein the C₁₋₄ aliphatic residue may be in each case be unsubstituted or mono- or polysubstituted; a NH(C₁₋₄ aliphatic residue), a N(C₁₋₄ aliphatic residue)₂, a NH—C(═O)—C₁₋₄ aliphatic residue, a NH—S(═O)₂-C₁₋₄-aliphatic residue, a N(C₁₋₄ aliphatic residue)-C(═O)-C₁₋₄ aliphatic residue, or a N(C₁₋₄ aliphatic residue)-S(═O)₂-C₁₋₄ aliphatic residue, wherein the C₁₋₄ aliphatic residue may in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted; R⁷ represents a C₁₋₁₀-aliphatic residue, unsubstituted or mono- or polysubstituted; a C₃₋₁₀-cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; on the condition that if R⁷ denotes a 3 to 10 membered heterocycloaliphatic residue, the 3 to 10 membered heterocycloaliphatic residue is linked via a carbon atom, in which an “aliphatic group” and “aliphatic residue” can in each case be branched or unbranched, saturated or unsaturated, in which a “cycloaliphatic residue” and a “heterocycloaliphatic residue” can in each case be saturated or unsaturated, in which “mono- or polysubstituted” with respect to an “aliphatic group” and an “aliphatic residue” relates, with respect to the corresponding residues or groups, to the substitution of one or more hydrogen atoms each independently of one another by at least one substituent selected from the group consisting of F, Cl, Br, I, NO₂, NH₂, an NH(C₁₋₄ aliphatic residue), a NH—C(═O)—C₁₋₄ aliphatic residue, a NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(═O)-N(C₁₋₄ aliphatic residue)₂; in which “mono- or polysubstituted” with respect to a “cycloaliphatic residue” and a “heterocycloaliphatic residue” relates, with respect to the corresponding residues, to the substitution of one or more hydrogen atoms each independently of one another by at least one substituent selected from the group consisting of F, Cl, Br, I, NO₂, NH₂, an NH(C₁₋₄ aliphatic residue), an N(C₁₋₄ aliphatic residue)₂, a NH—C(═O)—C₁₋₄ aliphatic residue, a NH—S(═O)₂-C₁₋₄ aliphatic residue, —0, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂—O—C₁₋₄-aliphatic residue, a S(═O)₂—NH—C₁₋₄-aliphatic residue, CN, CF₃, CHO, COOH, a C₁₋₄-aliphatic residue, a C(═O)—C₁₋₄-aliphatic residue, a C(═O)—O—C₁₋₄-aliphatic residue, a C₃₋₆-cycloaliphatic residue, a 3 to 6 membered heterocycloaliphatic residue, C(═O)—NH₂, a C(═O)—NH(C₁₋₄ aliphatic residue), and a C(—O)-N(C₁₋₄ aliphatic residue)₂; in which “mono- or polysubstituted” with respect to “aryl” and a “heteroaryl” relates, with respect to the corresponding residues, to the substitution of one or more hydrogen atoms each independently of one another by at least one substituent selected from the group consisting of F, Cl, Br, I, NO₂, NH₂,

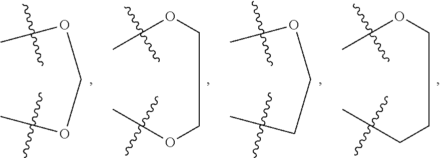 [SMILES: *OCO* ; *OCCO* ; *CCO* ; *CCCO*]

an NH(C₁₋₄ aliphatic residue), an N(C₁₋₄ aliphatic residue)₂, an NH—C(═O)—C₁₋₄ aliphatic residue, an NH—S(═O)₂-C₁₋₄ aliphatic residue, OH, OCF₃, a O—C₁₋₄-aliphatic residue, a O—C(═O)—C₁₋₄-aliphatic residue, SH, SCF₃, a S—C₁₋₄-aliphatic residue, S(═O)₂OH, a S(═O)₂-C₁₋₄-aliphatic residue, a S(═O)₂-O—C₁₋₄-aliphatic residue, a S(═O)₂—NH—C₁₋₄-aliphatic residue, CN, CF₃, C(═O) H, C(═O) OH, a C₁₋₄-aliphatic residue, a C(═O)-C₁₋₄-aliphatic residue, a C(—O)-O—C₁₋₄-aliphatic residue, a C₃₋₆-cycloaliphatic residue, a 3 to 6 membered heterocycloaliphatic residue, benzyl, aryl, heteroaryl, C(═O)—NH₂, a C(═O)—NH(C₁₋₄ aliphatic residue), and a C(═O)—N(C₁₋₄ aliphatic residue)₂; with the exception of the following compound: 1-ethyl-N-(4-methoxybenzyl)-4-methyl-2-oxo-1,2-dihydroquinoline-3-carboxamide, in the form of the free compounds, the racemate, the enantiomers, diastereomers, mixtures of the enantiomers or diastereomers in any mixing ratio, or of an individual enantiomer or diastereomer, or in the form of the salts of physiologically acceptable acids or bases.

In further embodiments, R¹ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄-aliphatic residue, OCF₃, SH, SCF₃, a S—C₁₋₄-aliphatic residue, CF₃, CN, a C₁₋₄-aliphatic residue and C(═O)—OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(—O)-OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

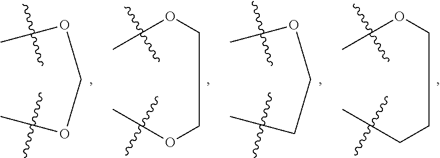 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, O—CH₂—OH, O—CH₂—O—CH₃, SH, SCF₃, a S—C₁₋₄ aliphatic residue, CF₃, CN, a C₁₋₄-aliphatic residue, C(—O)-OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH, and wherein the aryl or the heteroaryl residue may in each case be optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN and C(═O)—OH, X represents O or S; R² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄ aliphatic residue may be in each case be unsubstituted or mono- or polysubstituted; a C₃₋₆-cycloaliphatic residue or a 3 to 6 membered heterocycloaliphatic residue, in each case unsubstituted or mono- or polysubstituted and in each case optionally bridged via a C₁₋₄ aliphatic group, which in turn may be unsubstituted or mono- or polysubstituted, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)—C₁₋₄ aliphatic residue, a C(═O)—O—C₁₋₄ aliphatic residue, a C(═O)—NH—C₁₋₄ aliphatic residue, a C(═O)-N(C₁₋₄ aliphatic residue)₂, a O—C₁₋₄-aliphatic residue, a O—C(═O)—C₁₋₄-aliphatic residue, a S—C₁₋₄-aliphatic residue, a S(═O)₂-C₁₋₄-aliphatic residue, a NH(C₁₋₄ aliphatic residue), a N(C₁₋₄ aliphatic residue)₂, a NH—C(═O)—C₁₋₄ aliphatic residue, and a NH—S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, OH, a C₁₋₄-aliphatic residue and a O—C₁₋₄-aliphatic residue, and in each case optionally bridged via an unsubstituted C₁₋₄ aliphatic group, R⁷ denotes a C₁₋₁₀-aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄-aliphatic residue, OCF₃, SH, SCF₃, a S—C₁₋₄-aliphatic residue, CF₃, CN, a C₁₋₄-aliphatic residue, a C(═O)—O—C₁₋₄-aliphatic residue and C(—O) OH, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN, a C₁₋₄-aliphatic residue and C(═O)—OH, on the condition that if R⁷ denotes a 3 to 10 membered heterocycloaliphatic residue, the 3 to 10 membered heterocycloaliphatic residue is linked via a carbon atom.

In further embodiments, R² represents F; Cl; Br; I; CN; CF₃; NO₂; OCF₃; SCF₃; a C₁₋₄-aliphatic residue, a S—C₁₋₄-aliphatic residue, a O—C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted aliphatic residue, a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, wherein the C₁₋₄-aliphatic residue in each case may be unsubstituted or mono- or polysubstituted with at least one substituent selected from the group consisting of F, Cl, Br, I, OH, and an unsubstituted aliphatic residue, and wherein the C₃₋₆-cycloaliphatic residue or the 3 to 6 membered heterocycloaliphatic residue may in each case be optionally bridged via a C₁₋₄ aliphatic group, which in turn may be unsubstituted or mono- or polysubstituted with at least one substituent selected from the group consisting of F, Cl, Br, I, OH, an unsubstituted C₁₋₄-aliphatic residue and an unsubstituted O—C₁₋₄-aliphatic residue.

In further embodiments, R³, R⁴, R⁵ and R⁶ each independently of one another represent H; F; Cl; Br; I; CN; CF₃; OCF₃; SCF₃; C(═O) H; C(═O)—OH; C(═O)—NH₂; S(═O)₂—OH; NO₂; a C₁₋₄-aliphatic residue, a C(═O)-C₁₋₄ aliphatic residue, a C(═O)—O—C₁₋₄ aliphatic residue, a O—C₁₋₄-aliphatic residue, a O—C(═O)C₁₋₄-aliphatic residue, a S-C₁₋₄-aliphatic residue, a S(═O)₂-C₁₋₄-aliphatic residue, wherein the C₁₋₄-aliphatic residue in each case may be unsubstituted or mono- or polysubstituted with at least one substituent selected from the group consisting of F, Cl, Br, I, OH, and a O—C₁₋₄-aliphatic residue; a C₃₋₆-cycloaliphatic residue or a 3 to 6 membered heterocycloaliphatic residue, in each case unsubstituted or mono- or polysubstituted with at least one substituent selected from the group consisting of F, Cl, Br, I, ═O, OH, a C₁₋₄-aliphatic residue and a O—C₁₋₄-aliphatic residue, and in each case optionally bridged via an unsubstituted C₁₋₄ aliphatic group.

In further embodiments, R³, R⁴, R⁵ and R⁶ each independently of one another are selected from the group consisting of H; F; Cl; Br; I; NO₂; CF₃; CN; OCF₃; SCF₃; a (C═O)-C₁₋₄ aliphatic residue, a C₁₋₄ aliphatic residue, O—C₁₋₄ aliphatic residue, a S—C₁₋₄ aliphatic residue, wherein the C₁₋₄-aliphatic residue in each case may be unsubstituted or mono- or polysubstituted with at least one substituent selected from the group consisting of F, Cl, and O—CH₃.

In further embodiments, at least one of R³, R⁴, R⁵ and R⁶ is ≠H.

In further embodiments, R¹ represents the partial structure:

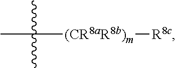

wherein, m denotes 0, 1, 2, 3 or 4, R^{8a} and R^{8b} each independently of one another represent H, F, Cl, Br, I, NO₂, NH₂, a NH(C₁₋₄ aliphatic residue), OH, an O—C₁₋₄ aliphatic residue, OCF₃, SH, SCF₃, a S—C₁₋₄ aliphatic residue, CF₃, CN, a C₁₋₄ aliphatic residue or C(═O)—OH, R^{8c} denotes a C₁₋₄ aliphatic residue, unsubstituted or mono- or polysubstituted with at least one substituent selected from the group consisting of F, Cl, Br, I, NO₂, NH₂, an NH(C₁₋₄ aliphatic residue), OH, an O—C₁₋₄ aliphatic residue, OCF₃, SH, SCF₃, a S—C₁₋₄ aliphatic residue, CF₃, CN, a C₁₋₄-aliphatic residue and C(═O)—OH, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, —O, an O—C₁₋₄ aliphatic residue, OCF₃, SH, SCF₃, a S—C₁₋₄ aliphatic residue, CF₃, CN, a C₁₋₄-aliphatic residue, C(═O)—OH, a C₃₋₆ cycloaliphatic residue and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, 4 aliphatic residue, OCF₃, SH, SCF₃, a S—C₁₋₄ aliphatic residue, CF₃, CN, a C₁₋₄-aliphatic residue and C(═O)—OH, or denotes an aryl or heteroaryl, in each case unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue,

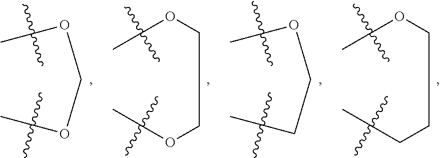 [SMILES: *OCO* ; *OCCO* ; *CCO* ; *CCCO*]

benzyl, phenyl, thienyl, pyridyl, furyl, thiazolyl and oxazolyl, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein benzyl, phenyl, thienyl, pyridyl, furyl, thiazolyl and oxazolyl may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, an O—C₁₋₄ aliphatic residue, OCF₃, SH, SCF₃, a S—C₁₋₄ aliphatic residue, CF₃, CN, a C₁₋₄-aliphatic residue, C(═O)—OH, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, NH₂, an NH(C₁₋₄ aliphatic residue), an N(C₁₋₄ aliphatic residue)₂, OH, ═O, an O—C₁₋₄ aliphatic residue, OCF₃, SH, SCF₃, a S—C₁₋₄ aliphatic residue, CF₃, CN, a C₁₋₄-aliphatic residue and C(═O)—OH.

In further embodiments, R¹ represents the partial structure:

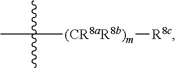

wherein: m denotes 0, 1, or 2, R^{8a} and R^{8b} each independently of one another represent H, F, Cl, Br, I, an O—C₁₋₄ aliphatic residue or a C₁₋₄ aliphatic residue, R^{8c} denotes a C₁₋₄ aliphatic residue, unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an O—C₁₋₄ aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, CF₃ and an unsubstituted O—C₁₋₄-aliphatic residue, or denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, a C₃₋₆ cycloaliphatic residue, a 3 to 6 membered heterocycloaliphatic residue, benzyl, phenyl, thienyl or pyridyl, wherein benzyl, phenyl, thienyl and pyridyl, may in each case may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃ and C(═O)—O—C₂H₅, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, OH, ═O, an O—C₁₋₄ aliphatic residue, OCF₃, CF₃ a C₁₋₄-aliphatic residue and C(═O)—OH.

In further embodiments, R⁷ denotes a C₁₋₁₀-aliphatic residue, unsubstituted or mono- or polysubstituted with at least one substituent selected from the group consisting of F, Cl, Br, I, NO₂, OH, an O—C₁₋₄-aliphatic residue, OCF₃, SH, SCF₃, a S—C₁₋₄-aliphatic residue, a C(═O)—O—C₁₋₄-aliphatic residue, COOH, CF₃, CN, and a C₁₋₄-aliphatic residue wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, a C(═O)—O—C₁₋₄-aliphatic residue, CF₃, CN, a C₁₋₄-aliphatic residue, a C₃₋₆ cycloaliphatic residue, and a 3 to 6 membered heterocycloaliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted O—C₁₋₄-aliphatic residue, and wherein the C₃₋₆ cycloaliphatic residue and the 3 to 6 membered heterocycloaliphatic residue may in each case may be unsubstituted or mono- or polysubstituted with at least one substituent selected from the group consisting of F, Cl, Br, I, NO₂, OH, ═O, an O—C₁₋₄ aliphatic residue, OCF₃, SH, SCF₃, a S—C₁₋₄ aliphatic residue, CF₃, CN, and a C₁₋₄-aliphatic residue, and wherein the C₃₋₁₀-cycloaliphatic residue or the 3 to 10 membered heterocycloaliphatic residue may in each case optionally bridged via a C₁₋₈ aliphatic group, which in turn may be unsubstituted or mono- or polysubstituted with at least one substituent selected from the group consisting of F, Cl, Br, I, NO₂, OH, an O—C₁₋₄ aliphatic residue, a C(═O)—O—C₁₋₄-aliphatic residue, OCF₃, SH, SCF₃, a S—C₁₋₄ aliphatic residue, CF₃, CN, and a C₁₋₄-aliphatic residue. on the condition that if R⁷ denotes a 3 to 10 membered heterocycloaliphatic residue, the 3 to 10 membered heterocycloaliphatic residue is linked via a carbon atom.

In further embodiments, R⁷ denotes a C₁₋₈-aliphatic residue, unsubstituted or mono- or polysubstituted with at least one substituent selected from the group consisting of F, Cl, Br, I, OH, an O—C₁₋₄-aliphatic residue, COOH, a C(═O)—O—C₁₋₄-aliphatic residue, OCF₃, SH, SCF₃, a S—C₁₋₄-aliphatic residue, CF₃, and a C₁₋₄-aliphatic residue wherein the C₁₋₄-aliphatic residue in each case may be unsubstituted or mono- or polysubstituted with at least one substituent selected from the group consisting of F, Cl, Br, I, OH, CF₃ and an unsubstituted aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, OH, ═O, an O—C₁₋₄ aliphatic residue, OCF₃, SCF₃, a S—C₁₋₄ aliphatic residue, a C(═O)—O—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of F, Cl, Br, I, OH, OCF₃, CF₃ and an unsubstituted C₁₋₄-aliphatic residue, and wherein the C₃₋₁₀-cycloaliphatic residue or the 3 to 10 membered heterocycloaliphatic residue in each case may be bridged via an unsubstituted C₁₋₈ aliphatic group, on the condition that if R⁷ denotes a 3 to 10 membered heterocycloaliphatic residue, the 3 to 10 membered heterocycloaliphatic residue is linked via a carbon atom.

In further embodiments, R¹ represents the partial structure:

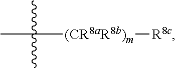

wherein: m is 0, 1 or 2 and R^{8a} and R^{8b} each independently of one another represent H, F, a O—C₁₋₄ aliphatic residue or a C₁₋₄ aliphatic residue; R^{8c} denotes a C₁₋₄ aliphatic residue, unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or denotes a C₃₋₁₀-cycloaliphatic residue or a 3 to 10 membered heterocycloaliphatic residue, in each case unsubstituted or mono- or polysubstituted with at least one substituent selected from the group consisting of F, Cl, Br, I, an unsubstituted O—C₁₋₄ aliphatic residue, CF₃, and an unsubstituted C₁₋₄-aliphatic residue, or wherein m is 0, R^{8a} and R^{8b} each independently of one another represent H, F, a O—C₁₋₄ aliphatic residue or a C₁₋₄ aliphatic residue; and R^{8c} denotes an aryl or heteroaryl, in each case unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(═O)—C₂H₅, C(═O)—O—CH₃, C(═O)—O—C₂H₅ and phenyl, wherein phenyl may be unsubstituted or mono- or polysubstituted with at least one substituent selected from the group consisting of F, Cl, Br, I, OH, an O—C₁₋₄ aliphatic residue, OCF₃, CF₃, CN, a C₁₋₄-aliphatic residue, C(═O)—CH₃, C(—O)-C₂H₅, C(═O)—O—CH₃ and C(═O)—O—C₂H₅, X represents O or S; R² is selected from the group consisting of F; Cl; Br; CF₃; CH₃; C₂H₅, iso-propyl; cyclopropyl; and O—CH₃; R³, R⁴, R⁵ and R⁶ are each independently of one another selected from the group consisting of H; F; Cl; Br; CF₃; CN; OCF₃ and NO₂; R⁷ denotes a C₁₋₆-aliphatic residue, unsubstituted or mono- or polysubstituted with at least one substituent selected from the group consisting of F, Cl, Br, I, OH, an O—C₁₋₄-aliphatic residue, COOH, a C(═O)—O—C₁₋₄-aliphatic residue, OCF₃, SH, SCF₃, a S—C₁₋₄-aliphatic residue, CF₃, and a C₁₋₄-aliphatic residue, wherein the C₁₋₄-aliphatic residue in each case may be unsubstituted or mono- or polysubstituted with at least one substituent selected from the group consisting of OH, and an unsubstituted O—C₁₋₄-aliphatic residue, or denotes an unsubstituted C₃₋₆-cycloaliphatic residue or an unsubstituted 3 to 6 membered heterocycloaliphatic residue, on the condition that if R⁷ denotes a 3 to 6 membered heterocycloaliphatic residue, the 3 to 6 membered heterocycloaliphatic residue is linked via a carbon atom.

In further embodiments, the Kv7 channel activator is selected from the group consisting of: N-[(3-Fluorophenyl)-methyl]-1,4-dimethyl-2-oxo-7-(trifluoromethyl)-1H-quinoline-3-carboxylic acid amide; 1-Butyl-N-[(3-fluorophenyl)-methyl]-4-methyl-2-oxo-7-(trifluoromethyl)-1H-quinoline-3-carboxylic acid amide; N-[(3-Fluorophenyl)-methyl]-1,4-dimethyl-2-thioxo-7-(trifluoromethyl)-1H-quinoline-3-carboxylic acid amide; N-[(3-Fluorophenyl)-methyl]-1-(2-methoxy-ethyl)-4-methyl-2-oxo-7-(trifluoromethyl)-1H-quinoline-3-carboxylic acid amide; 1-Ethyl-N-[(3-fluorophenyl)-methyl]-4-methyl-2-oxo-7-(trifluoromethyl)-1H-quinoline-3-carboxylic acid amide; N-[(4-Fluorophenyl)-methyl]-1,4-dimethyl-2-oxo-7-(trifluoromethyl)-1H-quinoline-3-carboxylic acid amide; N-[(4-Fluorophenyl)-methyl]-1-(2-methoxy-ethyl)-4-methyl-2-oxo-7-(trifluoromethyl)-1H-quinoline-3-carboxylic acid amide; 1-Ethyl-N-[(4-fluorophenyl)-methyl]-4-methyl-2-oxo-7-(trifluoromethyl)-1H-quinoline-3-carboxylic acid amide; N-[(3-Fluorophenyl)-methyl]-1,4-dimethyl-2-oxo-1H-quinoline-3-carboxylic acid amide; N-[(3-Fluorophenyl)-methyl]-1-(2-methoxy-ethyl)-4-methyl-2-oxo-1H-quinoline-3-carboxylic acid amide; N-[(3-Fluorophenyl)-methyl]-4-methyl-2-oxo-1-propyl-7-(trifluoromethyl)-1H-quinoline-3-carboxylic acid amide; N-[(3-Fluorophenyl)-methyl]-4-methyl-1-(3-methyl-butyl)-2-oxo-7-(trifluoromethyl)-1H-quinoline-3-carboxylic acid amide; N-[(3-Fluorophenyl)-methyl]-4-methyl-1-(4-methyl-pentyl)-2-oxo-7-(trifluoromethyl)-1H-quinoline-3-carboxylic acid amide; N-[(3-Fluorophenyl)-methyl]-1-(3-methoxy-propyl)-4-methyl-2-oxo-7-(trifluoromethyl)-1H-quinoline-3-carboxylic acid amide; N-[(3-Fluorophenyl)-methyl]-1-[2-(2-methoxy-ethoxy)-ethyl]-4-methyl-2-oxo-7-(trifluoromethyl)-1H-quinoline-3-carboxylic acid amide; 7-Bromo-N-[(3-fluorophenyl)-methyl]-1,4-dimethyl-2-oxo-1H-quinoline-3-carboxylic acid amide; 7-Bromo-N-[(4-fluorophenyl)-methyl]-1,4-dimethyl-2-oxo-1H-quinoline-3-carboxylic acid amide; 7-Bromo-N-[(3-fluorophenyl)-methyl]-1-(2-methoxy-ethyl)-4-methyl-2-oxo-1H-quinoline-3-carboxylic acid amide; 7-Bromo-N-[(4-fluorophenyl)-methyl]-1-(2-methoxy-ethyl)-4-methyl-2-oxo-1H-quinoline-3-carboxylic acid amide; 7-Cyano-N-[(3-fluorophenyl)-methyl]-1,4-dimethyl-2-oxo-1H-quinoline-3-carboxylic acid amide; 7-Cyano-N-[(4-fluorophenyl)-methyl]-1,4-dimethyl-2-oxo-1H-quinoline-3-carboxylic acid amide; 7-Cyano-N-[(3-fluorophenyl)-methyl]-1-(2-methoxy-ethyl)-4-methyl-2-oxo-1H-quinoline-3-carboxylic acid amide; 7-Cyano-N-[(4-fluorophenyl)-methyl]-1-(2-methoxy-ethyl)-4-methyl-2-oxo-1H-quinoline-3-carboxylic acid amide; N-(4,4-Dimethyl-pentyl)-1-(2-methoxy-ethyl)-4-methyl-2-oxo-7-(trifluoromethyl)-1H-quinoline-3-carboxylic acid amide; N-(4,4-Dimethyl-pentyl)-1,4-dimethyl-2-oxo-7-(trifluoromethyl)-1H-quinoline-3-carboxylic acid amide; 2-[3-[(3-Fluorophenyl)-methyl-carbamoyl]-4-methyl-2-oxo-7-(trifluoromethyl)-1H-quinolin-1-yl]-acetic acid methyl ester; 3-[3-[(3-Fluorophenyl)-methyl-carbamoyl]-4-methyl-2-oxo-7-(trifluoromethyl)-1H-quinolin-1-yl]-propionic acid methyl ester; 2-[3-[(3-Fluorophenyl)-methyl-carbamoyl]-4-methyl-2-oxo-7-(trifluoromethyl)-1H-quinolin-1-yl]-acetic acid; 3-[3-[(3-Fluorophenyl)-methyl-carbamoyl]-4-methyl-2-oxo-7-(trifluoromethyl)-1H-quinolin-1-yl]-propionic acid; N-[(3-Fluorophenyl)-methyl]-1-[1-(methoxymethyl)-propyl]-4-methyl-2-oxo-7-(trifluoromethyl)-1H-quinoline-3-carboxylic acid amide; N-[(3-Fluorophenyl)-methyl]-1-[2-methoxy-1-(methoxymethyl)-ethyl]-4-methyl-2-oxo-7-(trifluoromethyl)-1H-quinoline-3-carboxylic acid amide; N-[(3-Fluorophenyl)-methyl]-1-(2-methoxy-butyl)-4-methyl-2-oxo-7-(trifluoromethyl)-1H-quinoline-3-carboxylic acid amide; N-[(3-Fluorophenyl)-methyl]-1,4-dimethyl-2-oxo-7-(trifluoromethyloxy)-1H-quinoline-3-carboxylic acid amide; 7-Fluoro-N-[(3-fluorophenyl)-methyl]-1,4-dimethyl-2-oxo-1H-quinoline-3-carboxylic acid amide; N-[(3-Fluorophenyl)-methyl]-1-(2-methoxy-1-methyl-ethyl)-4-methyl-2-oxo-7-(trifluoromethyl)-1H-quinoline-3-carboxylic acid amide; N-[(4-Fluorophenyl)-methyl]-1,4-dimethyl-2-thioxo-7-(trifluoromethyl)-1H-quinoline-3-carboxylic acid amide; 7-Chloro-N-[(3-fluorophenyl)-methyl]-1,4-dimethyl-2-oxo-1H-quinoline-3-carboxylic acid amide; N-[(3-Fluorophenyl)-methyl]-1-(2-hydroxy-ethyl)-4-methyl-2-oxo-7-(trifluoromethyl)-1H-quinoline-3-carboxylic acid amide; 1-(2-Ethoxy-ethyl)-N-[(3-fluorophenyl)-methyl]-4-methyl-2-oxo-7-(trifluoromethyl)-1H-quinoline-3-carboxylic acid amide; N-[(3-Fluorophenyl)-methyl]-1-isopropyl-4-methyl-2-oxo-7-(trifluoromethyl)-1H-quinoline-3-carboxylic acid amide; N-[(3-Fluorophenyl)-methyl]-4-methyl-2-oxo-1-pentyl-7-(trifluoromethyl)-1H-quinoline-3-carboxylic acid amide; N-[(3-Fluorophenyl)-methyl]-1-methyl-2-oxo-4-(trifluoromethyl)-1H-quinoline-3-carboxylic acid amide; N-[(3-Fluorophenyl)-methyl]-1-(2-methoxy-propyl)-4-methyl-2-oxo-7-(trifluoromethyl)-1H-quinoline-3-carboxylic acid amide; N-[(3-Fluorophenyl)-methyl]-4-methyl-2-oxo-1-tetrahydro-pyran-4-yl-7-(trifluoromethyl)-1H-quinoline-3-carboxylic acid amide; and N-[(3-Fluorophenyl)-methyl]-4-methoxy-1-methyl-2-oxo-7-(trifluoromethyl)-1H-quinoline-3-carboxylic acid amide; respectively in the form of the free compounds; the racemate; the enantiomers, diastereomers, mixtures of the enantiomers or diastereomers in any mixing ratio or of an individual enantiomer or diastereomer; or in the form of the salts of physiologically acceptable acids or bases.

### Formula 30

In an embodiment, the Kv7 channel activator may be selected from one of the following compounds according to Formula 30. Such compounds are described in International Publication No. WO2010075973A1, published Jul. 8, 2010, and corresponding to International Application No. PCT/EP2009/009040 filed Dec. 16, 2009; U.S. Pat. No. 8,367,700 issued Feb. 5, 2013 and corresponding to U.S. application Ser. No. 12/635,800 filed Dec. 11, 2009; which are incorporated by reference in their entirety herein. In the case of any conflict of terminology in the context of Formula 30, these references incorporated by reference herein control.

In an embodiment, the Kv7 channel activator is a compound according to formula 30:

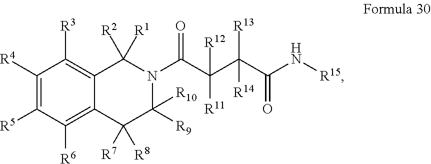

wherein, R⁰ stands for C₁₋₁₀ alkyl or C₂₋₁₀ heteroalkyl, each saturated or unsaturated, branched or unbranched, unsubstituted or mono- or polysubstituted; C₃₋₇ cycloalkyl or heterocyclyl, each saturated or unsaturated, unsubstituted or mono- or polysubstituted; aryl or heteroaryl, each unsubstituted or mono- or polysubstituted; C₁₋₈ alkyl-bridged C₃₋₇ cycloalkyl or heterocyclyl, each saturated or unsaturated, unsubstituted or mono- or polysubstituted, wherein the alkyl chain can in each case be branched or unbranched, saturated or unsaturated, unsubstituted, mono- or polysubstituted; or C₁₋₈ alkyl-bridged aryl or heteroaryl, each unsubstituted or mono- or polysubstituted, wherein the alkyl chain can in each case be branched or unbranched, saturated or unsaturated, unsubstituted, mono- or polysubstituted; R¹ stands for F; Cl; Br; CN; C₁₋₁₀ alkyl or C₂₋₁₀ heteroalkyl, each saturated or unsaturated, branched or unbranched, unsubstituted or mono- or polysubstituted; C₃₋₇ cycloalkyl or heterocyclyl, each saturated or unsaturated, unsubstituted or mono- or polysubstituted; aryl or heteroaryl, each unsubstituted or mono- or polysubstituted; C₁₋₈ alkyl-bridged C₃₋₇ cycloalkyl or heterocyclyl, each saturated or unsaturated, unsubstituted or mono- or polysubstituted, wherein the alkyl chain can in each case be branched or unbranched, saturated or unsaturated, unsubstituted, mono- or polysubstituted; C₁₋₈ alkyl-bridged heteroaryl, unsubstituted or mono- or polysubstituted, wherein the alkyl chain can be branched or unbranched, saturated or unsaturated, unsubstituted, mono- or polysubstituted; or C₁₋₈ alkyl-bridged aryl, unsubstituted or mono- or polysubstituted, wherein the alkyl chain can be unbranched, saturated or unsaturated, unsubstituted; R² stands for H; F; Cl; Br; or C₁₋₁₀ alkyl, saturated or unsaturated, branched or unbranched, unsubstituted or mono- or polysubstituted; or R¹ and R² together with the carbon atom binding them as ring member form a C₃₋₇ cycloalkyl or heterocyclyl, each saturated or unsaturated, unsubstituted or mono- or polysubstituted, each optionally fused to (hetero) aryl, unsubstituted or mono- or polysubstituted; R³, R⁴, R⁵ and R⁶ each mutually independently denote H; F; Cl; Br; I; NO₂; CF₃; CN; R⁰; C(═O) H; C(═O)R⁰; CO₂H; C(═O) OR⁰; CONH₂; C(═O)NHR⁰; C(═O)N(R⁰)₂; OH; OR⁰; —O—(C₁₋₈ alkyl)-O—; O—C(═O)—R⁰; O—C(═O)—O—R⁰; O—(C═O)—NH—R⁰; O—C(═O)—N(R⁰)₂; O—S(═O)₂-R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰C(═O)—R⁰; NR⁰—C(═O)—O—R⁰; NR⁰C(═O)—NH₂; NR⁰C(═O)—NH—R⁰; NR⁰C(═O)—)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NRO—S(═O)₂OH; NRO—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂; R⁷, R⁸, R⁹, R¹⁰ each mutually independently stand for H; F; Cl; Br; or C₁₋₁₀ alkyl, saturated or unsaturated, branched or unbranched, unsubstituted or mono- or polysubstituted; R¹¹ stands for H; F; Cl; Br; CN; or R⁰; R¹² stands for H; F; Cl; Br; CN; or C₁₋₁₀ alkyl, saturated or unsaturated, branched or unbranched, unsubstituted or mono- or polysubstituted; R¹³ stands for H; F; Cl; Br; CN; C₁₋₁₀ alkyl or C₂₋₁₀ heteroalkyl, each saturated or unsaturated, branched or unbranched, unsubstituted or mono- or polysubstituted; C₃₋₇ cycloalkyl or heterocyclyl, each saturated or unsaturated, unsubstituted or mono- or polysubstituted; aryl or heteroaryl, each unsubstituted or mono- or polysubstituted; C₁₋₈ alkyl-bridged C₃₋₇ cycloalkyl or heterocyclyl, each saturated or unsaturated, unsubstituted or mono- or polysubstituted, wherein the alkyl chain can in each case be branched or unbranched, saturated or unsaturated, unsubstituted, mono- or polysubstituted; or C₂₋₈ alkyl-bridged aryl or heteroaryl, each unsubstituted or mono- or polysubstituted, wherein the alkyl chain can in each case be branched or unbranched, saturated or unsaturated, unsubstituted, mono- or polysubstituted; R¹⁴ stands for H; F; Cl; Br; CN; or C₁₋₁₀ alkyl, saturated or unsaturated, branched or unbranched, unsubstituted or mono- or polysubstituted; or R¹¹ and R¹³ together with the carbon atoms binding them as ring members form a C₃₋₇ cycloalkyl or heterocyclyl, each saturated or unsaturated, unsubstituted or mono- or polysubstituted, optionally fused to (hetero) aryl, unsubstituted or mono- or polysubstituted; or R¹¹ and R¹²; or R¹³ and R¹⁴, together with the carbon atoms binding them as ring members form a C₃₋₇ cycloalkyl or heterocyclyl, each saturated or unsaturated, unsubstituted or mono- or polysubstituted, each optionally fused to (hetero) aryl, unsubstituted or mono- or polysubstituted; R¹⁵ stands for R⁰; wherein “alkyl-substituted”, “heteroalkyl-substituted”, “heterocyclyl-substituted” and “cycloalkyl-substituted” stand for the substitution of one or more hydrogen atoms, each mutually independently, with F; Cl; Br; I; CN; CF₃; ═O; ═NH; ═C(NH₂)₂; NO₂; R⁰; C(═O) H; C(═O)R⁰; CO₂H; C(═O) OR⁰; CONH₂; C(═O)NHR⁰; C(═O)N(R⁰)₂; OH; OR⁰; O—(C₁₋₈ alkyl)-O; O—C(═O)—R⁰; O—C(═O)—O—R⁰; O—(C═O)—NH—R⁰; O—C(═O)—N(R⁰)₂; O—S(═O)₂-R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH C(═O)—O—R⁰; NH—C(═O)NH₂; NH—C(═O)—NH—R⁰; NH—C(═O)—N(R⁰)₂; NR⁰—C(═O)—R⁰; NR⁰C(═O)—O—R⁰; NR⁰C(═O)—NH₂; NR⁰C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R)₂; NRO—S(═O)₂OH; NRO—S(═O)₂R⁰; NR⁰—S(═O)₂OR⁰; NR⁰—S(═O)₂NH₂; NR⁰—S(═O)₂NHR⁰; NRO—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂; wherein “aryl-substituted” and “heteroaryl-substituted” stand for the substitution of one or more hydrogen atoms, each mutually independently, with F; Cl; Br; I; NO₂; CF₃; CN; R⁰; C(═O) H; C(═O)R⁰; CO₂H; C(═O) OR⁰; CONH₂; C(═O)NHR⁰; C(═O)N(R⁰)₂; OH; OR⁰; O—(C₁₋₈ alkyl)-O; O—C(═O)—R⁰; O—C(═O)—O—R⁰; O—(C═O)—NH—R⁰; O—C(═O)—N(R⁰)₂; O—S(═O)₂-R⁰; O—S(═O)₂₀H; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)R⁰; NH—C(═O)—0-R⁰; NH—C(═O)—NH₂; NH—C(═O)—NH—R⁰; NH—C(═O)—N(R⁰)₂; NR⁰—C(═O)—R⁰; NR⁰C(═O)—O—R⁰; NR⁰—C(═O)—NH₂; NR⁰C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂; in the form of a free compound or salt of a physiologically compatible acid or base.

In further embodiments, R¹ stands for C₁₋₁₀ alkyl or C₂₋₁₀ heteroalkyl, each saturated or unsaturated, branched or unbranched, unsubstituted or mono- or polysubstituted; C₃₋₇ cycloalkyl, saturated or unsaturated, unsubstituted or mono- or polysubstituted; aryl or heteroaryl, each unsubstituted or mono- or polysubstituted; C₁₋₈ alkyl-bridged C₃₋₇ cycloalkyl, saturated or unsaturated, unsubstituted or mono- or polysubstituted, wherein the alkyl chain can be branched or unbranched, saturated or unsaturated, unsubstituted, mono- or polysubstituted; C₁₋₈ alkyl-bridged heteroaryl, unsubstituted or mono- or polysubstituted, wherein the alkyl chain can be branched or unbranched, saturated or unsaturated, unsubstituted, mono- or polysubstituted; or C₁₋₈ alkyl-bridged aryl, unsubstituted or mono- or polysubstituted, wherein the alkyl chain can be unbranched, saturated or unsaturated, unsubstituted; R² stands for H; or C₁₋₁₀ alkyl, saturated or unsaturated, branched or unbranched, unsubstituted or mono- or polysubstituted.

In further embodiments, R³, R⁴, R⁵ and R⁶ each mutually independently denote H; F; Cl; Br; I; NO₂; CF₃; CN; R⁰; C(═O) (R⁰ or H); C(═O) O(R⁰ or H); C(═O)N(R⁰ or H)₂; OH; OR⁰; —O—(C₁₋₈ alkyl)-O—; O—(C₁₋₈ alkyl)-O—C₁₋₈ alkyl; OCF₃; N(R⁰ or H)₂; N(R⁰ or H)C(═O)R⁰; N(R⁰ or H)—C(═O)—N(R⁰ or H)₂; SH; SCF₃; SR⁰; S(═O)₂R⁰; S(═O)₂O(R⁰ or H); S(═O)₂—N(R⁰ or H)₂.

In further embodiments, R⁷, R⁸, R⁹, R¹⁰ mutually independently stand for H; or C₁₋₁₀ alkyl, saturated or unsaturated, branched or unbranched, unsubstituted or mono- or polysubstituted.

In further embodiments, R¹¹ stands for H; F; Cl; Br; CN; C₁₋₁₀ alkyl, saturated or unsaturated, branched or unbranched, unsubstituted or mono- or polysubstituted; phenyl or heteroaryl, each unsubstituted or mono- or polysubstituted; or C₁₋₄ alkyl-bridged phenyl or heteroaryl, each unsubstituted or mono- or polysubstituted, wherein the alkyl chain can in each case be branched or unbranched, saturated or unsaturated, unsubstituted or mono- or polysubstituted; R¹³ stands for H; F; Cl; Br; CN; C₁₋₁₀ alkyl, saturated or unsaturated, branched or unbranched, unsubstituted or mono- or polysubstituted; phenyl or heteroaryl, each unsubstituted or mono- or polysubstituted; or C₂₋₄ alkyl-bridged phenyl or heteroaryl, each unsubstituted or mono- or polysubstituted, wherein the alkyl chain can in each case be branched or unbranched, saturated or unsaturated, unsubstituted or mono- or polysubstituted; or R¹¹ and R¹³ together with the carbon atoms binding them as ring members form a C₃₋₇ cycloalkyl or heterocyclyl, each saturated or unsaturated, unsubstituted or mono- or polysubstituted, optionally fused to phenyl, unsubstituted or mono- or polysubstituted.

In further embodiments, R¹² and R¹⁴ each mutually independently stand for H; or C₁₋₁₀ alkyl, saturated or unsaturated, branched or unbranched, unsubstituted or mono- or polysubstituted.

In further embodiments, R¹⁵ stands for C₃₋₁₀ alkyl or C₂₋₁₀ heteroalkyl, each saturated or unsaturated, branched or unbranched, unsubstituted or mono- or polysubstituted; C₃₋₇ cycloalkyl, saturated or unsaturated, unsubstituted or mono- or polysubstituted; aryl or heteroaryl, unsubstituted or mono- or polysubstituted; C₁₋₈ alkyl-bridged C₃₋₇ cycloalkyl, saturated or unsaturated, unsubstituted or mono- or polysubstituted, wherein the alkyl chain can in each case be branched or unbranched, saturated or unsaturated, unsubstituted, mono- or polysubstituted; or C₁₋₈ alkyl-bridged aryl or heteroaryl, each unsubstituted or mono- or polysubstituted, wherein the alkyl chain can in each case be branched or unbranched, saturated or unsaturated, unsubstituted, mono- or polysubstituted.

### Formula 31

In an embodiment, the Kv7 channel activator may be selected from one of the following compounds according to Formula 31. Such compounds are described in International Publication No. WO2010075973A1, published Jul. 8, 2010, and corresponding to International Application No. PCT/EP2009/009040 filed Dec. 16, 2009; U.S. Pat. No. 8,367,700 issued Feb. 5, 2013, and corresponding to U.S. application Ser. No. 12/635,800 filed Dec. 11, 2009; which are incorporated by reference in their entirety herein. In the case of any conflict of terminology in the context of Formula 31, these references incorporated by reference herein control.

In an embodiment, the Kv7 channel activator is a compound according to formula 31:

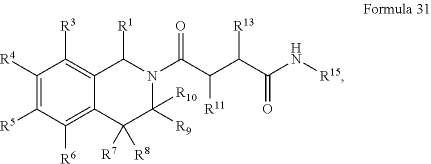

wherein, R¹ stands for C₁₋₁₀ alkyl or C₂₋₁₀ heteroalkyl, each saturated or unsaturated, branched or unbranched, unsubstituted or mono- or polysubstituted; C₃₋₇ cycloalkyl, saturated or unsaturated, unsubstituted or mono- or polysubstituted; phenyl or thienyl, each unsubstituted or mono- or polysubstituted; C₁₋₈ alkyl-bridged C₃₋₇ cycloalkyl, saturated or unsaturated, unsubstituted or mono- or polysubstituted; C₁₋₈ alkyl-bridged phenyl, unsubstituted or mono- or polysubstituted, wherein the alkyl chain can be branched or unbranched, saturated or unsaturated, unsubstituted; or C₁₋₈ alkyl-bridged thienyl, unsubstituted or mono- or polysubstituted, wherein the alkyl chain can be branched or unbranched, saturated or unsaturated, unsubstituted or mono- or polysubstituted; R³, R⁴, R⁵ and R⁶ each mutually independently denote H; F; Cl; Br; I; NO₂; CF₃; CN; R⁰; C(═O) (R⁰ or H); C(═O) O(R⁰ or H); C(═O)N(R⁰ or H)₂; OH; OR⁰; O—(C₁₋₈ alkyl)-O; O—(C₁₋₈ alkyl)-O—C₁₋₈ alkyl; N(R⁰ or H)₂; N(R⁰ or H)-C(═O)—R⁰; N(R⁰ or H)-C(═O)—N(R⁰ or H)₂; SH; SR⁰; S(═O)₂R⁰; S(═O)₂O(R⁰ or H); or S(═O)₂—N(R⁰ or H)₂; R⁷, R⁸, R⁹, R¹⁰ each mutually independently stand for H; or C₁₋₄ alkyl, saturated or unsaturated, branched or unbranched, unsubstituted or mono- or polysubstituted; R¹¹ and R¹³ each independently stand for H; or C₁₋₁₀ alkyl, saturated or unsaturated, branched or unbranched, unsubstituted or mono- or polysubstituted; or R¹¹ and R¹³ together with the carbon atoms binding them as ring members form a C₃₋₇ cycloalkyl or heterocyclyl, each saturated or unsaturated, unsubstituted or mono- or polysubstituted; R¹⁵ stands for C₃₋₁₀ alkyl, saturated or unsaturated, branched or unbranched, unsubstituted or mono- or polysubstituted; C₃₋₇ cycloalkyl, saturated or unsaturated, unsubstituted or mono- or polysubstituted; aryl or heteroaryl, unsubstituted or mono- or polysubstituted; C₁₋₈ alkyl-bridged C₃₋₇ cycloalkyl, saturated or unsaturated, unsubstituted or mono- or polysubstituted, wherein the alkyl chain can in each case be branched or unbranched, saturated or unsaturated, unsubstituted, mono- or polysubstituted; or C₁₋₈ alkyl-bridged aryl or heteroaryl, each unsubstituted or mono- or polysubstituted, wherein the alkyl chain can in each case be branched or unbranched, saturated or unsaturated, unsubstituted, mono- or polysubstituted.

In further embodiments, R¹⁵ stands for C₃₋₁₀ alkyl, saturated or unsaturated; branched or unbranched, unsubstituted or mono- or polysubstituted; or is selected from the following substructures:

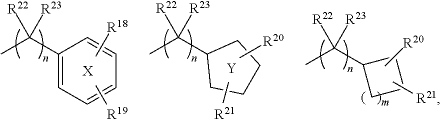

wherein: n=0, 1, 2, 3, 4, 5, 6, 7 or 8; m=0, 1, 2 or 3; ring X can contain one or two N atoms as ring member(s); ring Y contains at least 1 heteroatom selected from N, O or S and can contain up to 3 heteroatoms mutually independently selected from N, O or S; and/or can contain one or two double bonds; R¹⁸ and R¹⁹ mutually independently denote H; F; Cl; Br; I; NO₂; CF₃; CN; R⁰; C(═O) (R⁰ or H); C(═O) O(R⁰ or H); C(═O)N(R⁰ or H)₂; OH; OR⁰; O—(C₁₋₈ alkyl)-O; O—(C₁₋₈ alkyl)-O—C₁₋₈ alkyl; N(R⁰ or H)₂; N(R⁰ or H)—C(═O)—R⁰; N(R⁰ or H)—C(═O)—N(R⁰ or H)₂; SH; SR⁰; S(═O)₂R⁰; S(═O)₂O(R⁰ or H); or S(═O)₂—N(R⁰ or H)₂H; or R¹⁸ and R¹⁹ together with the carbon or nitrogen atoms binding them as ring members form an aryl or heteroaryl fused to the phenyl or heteroaryl ring, each unsubstituted or mono- or polysubstituted; or a C₃₋₇ cycloalkyl or heterocyclyl fused to the phenyl or heteroaryl ring, each saturated or unsaturated, unsubstituted or mono- or polysubstituted; R²⁰ and R²¹ mutually independently denote H or C₁₋₁₀ alkyl, saturated or unsaturated; branched or unbranched, unsubstituted or mono- or polysubstituted; or C₃₋₇ cycloalkyl or heterocyclyl, each saturated or unsaturated, unsubstituted or mono- or polysubstituted; or R²⁰ and R²¹ together with the carbon atoms or heteroatoms binding them as ring members form an aryl or heteroaryl fused to ring Y, each unsubstituted or mono- or polysubstituted; R²² and R²³ mutually independently denote H; or C₁₋₁₀ alkyl, saturated or unsaturated; branched or unbranched, unsubstituted.

In further embodiments, the Kv7 channel activator is selected from the group consisting of: 4-Oxo-4-(1-phenyl-3,4-dihydroisoquinolin-2 (1H)-yl)-N-(3-(trifluoromethyl)benzyl) butyric acid amide; 4-(1-Methyl-3,4-dihydro-1H-isoquinolin-2-yl)-4-oxo-N-[[3-(trifluoromethyl)phenyl]methyl]butyric acid amide; 4-Oxo-4-(1-thien-2-yl-3,4-dihydroisoquinolin-2 (1H)-yl)-N-(3-(trifluoromethyl)benzyl) butyric acid amide; 4-Oxo-4-[[1-(4-pyridyl)-3,4-dihydro-1H-isoquinolin-2-yl]-N-[3-(trifluoromethyl)phenyl]methyl]butyric acid amide; 4-(7-Fluoro-1-phenyl-3,4-dihydroisoquinolin-2 (1H)-yl)-4-oxo-N-(3-(trifluoromethyl)benzyl) butyric acid amide; 4-(5-Fluoro-1-phenyl-3,4-dihydro-1H-isoquinolin-2-yl)-4-oxo-N-[[3-(trifluoromethyl)phenyl]methyl]butyric acid amide; 4-Oxo-4-(1-phenyl-3,4-dihydro-1H-isoquinolin-2-yl)-N-[[2-(trifluoromethyl)phenyl]methyl]butyric acid amide; 4-Oxo-4-(1-phenyl-3,4-dihydro-1H-isoquinolin-2-yl)-N-[[4-(trifluoromethyl)phenyl]methyl]butyric acid amide; 4-(4-Methyl-1-phenyl-3,4-dihydro-1H-isoquinolin-2-yl)-4-oxo-N-[[3-(trifluoromethyl)phenyl]methyl]butyric acid amide; 4-(4,4-Dimethyl-1-phenyl-1,3-dihydroisoquinolin-2-yl)-4-oxo-N-[[3-(trifluoromethyl)phenyl]methyl]butyric acid amide; 4-(7-Methoxy-1-phenyl-3,4-dihydro-1H-isoquinolin-2-yl)-4-oxo-N-[[3-(trifluoromethyl)phenyl]methyl]butyric acid amide; 4-(5-Methoxy-1-phenyl-3,4-dihydro-1H-isoquinolin-2-yl)-4-oxo-N-[[3-(trifluoromethyl)phenyl]methyl]butyric acid amide; 4-(3-Methyl-1-phenyl-3,4-dihydro-1H-isoquinolin-2-yl)-4-oxo-N-[[3-(trifluoromethyl)phenyl]methyl]butyric acid amide; N-(2-Chlorophenyl)-4-oxo-4-(1-phenyl-3,4-dihydro-1H-isoquinolin-2-yl) butyric acid amide; N-(2,1,3-Benzothiadiazol-4-yl)-4-oxo-4-(1-phenyl-3,4-dihydro-1H-isoquinolin-2-yl) butyric acid amide; N-(1-Methyl-6-indazolyl)-4-oxo-4-(1-phenyl-3,4-dihydro-1H-isoquinolin-2-yl) butyric acid amide; N-(2-Furylmethyl)-4-oxo-4-(1-phenyl-3,4-dihydro-1H-isoquinolin-2-yl) butyric acid amide; N-Benzyl-4-oxo-4-(1-phenyl-3,4-dihydro-1H-isoquinolin-2-yl) butyric acid amide; 4-Oxo-4-(1-phenyl-3,4-dihydro-1H-isoquinolin-2-yl)-N-(2-pyridylmethyl) butyric acid amide; N-[(4-Methoxyphenyl)methyl]-4-oxo-4-(1-phenyl-3,4-dihydro-1H-isoquinolin-2-yl) butyric acid amide; 4-Oxo-N-phenethyl-4-(1-phenyl-3,4-dihydro-1H-isoquinolin-2-yl) butyric acid amide; 4-Oxo-4-(1-phenyl-3,4-dihydro-1H-isoquinolin-2-yl)-N-(4-pyridylmethyl) butyric acid amide; 4-Oxo-4-(1-phenyl-3,4-dihydro-1H-isoquinolin-2-yl)-N-(3-phenylpropyl) butyric acid amide; N-[2-(1H-Indol-3-yl)ethyl]-4-oxo-4-(1-phenyl-3,4-dihydro-1H-isoquinolin-2-yl) butyric acid amide; 4-(5,7-Dimethyl-1-phenyl-3,4-dihydro-1H-isoquinolin-2-yl)-N-(4-methoxyphenyl)-4-oxobutyric acid amide; N-(2-Chlorophenyl)-4-(5,7-dimethyl-1-phenyl-3,4-dihydro-1H-isoquinolin-2-yl)-4-oxobutyric acid amide; -(5,7-Dimethyl-1-phenyl-3,4-dihydro-1H-isoquinolin-2-yl)-N-(1-methyl-6-indazolyl)-4-oxobutyric acid amide; 4-(5,7-Dimethyl-1-phenyl-3,4-dihydro-1H-isoquinolin-2-yl)-N-(2-furylmethyl)-4-oxobutyric acid amide; N-Benzyl-4-(5,7-dimethyl-1-phenyl-3,4-dihydro-1H-isoquinolin-2-yl)-4-oxobutyric acid amide; 4-(5,7-Dimethyl-1-phenyl-3,4-dihydro-1H-isoquinolin-2-yl)-4-oxo-N-(2-pyridylmethyl) butyric acid amide; 4-(5,7-Dimethyl-1-phenyl-3,4-dihydro-1H-isoquinolin-2-yl)-N-[(4-methoxyphenyl)methyl]-4-oxobutyric acid amide; 4-(5,7-Dimethyl-1-phenyl-3,4-dihydro-1H-isoquinolin-2-yl)-4-oxo-N-phenethylbutyric acid amide; 4-(5,7-Dimethyl-1-phenyl-3,4-dihydro-1H-isoquinolin-2-yl)-4-oxo-N-(3-phenylpropyl) butyric acid amide; N-(4-Methoxyphenyl)-4-(5-methyl-1-phenyl-3,4-dihydro-1H-isoquinolin-2-yl)-4-oxobutyric acid amide; N-(2-Chlorophenyl)-4-(5-methyl-1-phenyl-3,4-dihydro-1H-isoquinolin-2-yl)-4-oxobutyric acid amide; N-(1-Methyl-6-indazolyl)-4-(5-methyl-1-phenyl-3,4-dihydro-1H-isoquinolin-2-yl)-4-oxobutyric acid amide; N-(2-Furylmethyl)-4-(5-methyl-1-phenyl-3,4-dihydro-1H-isoquinolin-2-yl)-4-oxobutyric acid amide; N-Benzyl-4-(5-methyl-1-phenyl-3,4-dihydro-1H-isoquinolin-2-yl)-4-oxobutyric acid amide; 4-(5-Methyl-1-phenyl-3,4-dihydro-1H-isoquinolin-2-yl)-4-oxo-N-(2-pyridylmethyl) butyric acid amide; N-[(4-Methoxyphenyl)methyl]-4-(5-methyl-1-phenyl-3,4-dihydro-1H-isoquinolin-2-yl)-4-oxobutyric acid amide; 4-(5-Methyl-1-phenyl-3,4-dihydro-1H-isoquinolin-2-yl)-4-oxo-N-phenethylbutyric acid amide; 4-(5-Methyl-1-phenyl-3,4-dihydro-1H-isoquinolin-2-yl)-4-oxo-N-(3-phenylpropyl) butyric acid amide; N-(4-Methoxyphenyl)-4-(7-methyl-1-phenyl-3,4-dihydro-1H-isoquinolin-2-yl)-4-oxobutyric acid amide; N-(2-Chlorophenyl)-4-(7-methyl-1-phenyl-3,4-dihydro-1H-isoquinolin-2-yl)-4-oxobutyric acid amide; N-(1-Methyl-6-indazolyl)-4-(7-methyl-1-phenyl-3,4-dihydro-1H-isoquinolin-2-yl)-4-oxobutyric acid amide; N-(2-Furylmethyl)-4-(7-methyl-1-phenyl-3,4-dihydro-1H-isoquinolin-2-yl)-4-oxobutyric acid amide; N-Benzyl-4-(7-methyl-1-phenyl-3,4-dihydro-1H-isoquinolin-2-yl)-4-oxobutyric acid amide; 4-(7-Methyl-1-phenyl-3,4-dihydro-1H-isoquinolin-2-yl)-4-oxo-N-(2-pyridylmethyl) butyric acid amide; N-[(4-Methoxyphenyl)methyl]-4-(7-methyl-1-phenyl-3,4-dihydro-1H-isoquinolin-2-yl)-4-oxobutyric acid amide; 4-(7-Methyl-1-phenyl-3,4-dihydro-1H-isoquinolin-2-yl)-4-oxo-N-phenethylbutyric acid amide; 4-(7-Methyl-1-phenyl-3,4-dihydro-1H-isoquinolin-2-yl)-4-oxo-N-(4-pyridylmethyl) butyric acid amide; 4-Oxo-4-(1-phenyl-3,4-dihydro-1H-isoquinolin-2-yl)-N-(2-thienylmethyl) butyric acid amide; N-[(2-Chlorophenyl)methyl]-4-oxo-4-(1-phenyl-3,4-dihydro-1H-isoquinolin-2-yl) butyric acid amide; N-[(2,4-Dichlorophenyl)methyl]-4-oxo-4-(1-phenyl-3,4-dihydro-1H-isoquinolin-2-yl) butyric acid amide; N-[(3,4-Dichlorophenyl)methyl]-4-oxo-4-(1-phenyl-3,4-dihydro-1H-isoquinolin-2-yl) butyric acid amide; 4-Oxo-4-(1-phenyl-3,4-dihydro-1H-isoquinolin-2-yl)-N-(p-tolylmethyl) butyric acid amide; N-(1,3-Benzodioxol-5-ylmethyl)-4-oxo-4-(1-phenyl-3,4-dihydro-1H-isoquinolin-2-yl) butyric acid amide; N-[(3-Fluorophenyl)methyl]-4-oxo-4-(1-phenyl-3,4-dihydro-1H-isoquinolin-2-yl) butyric acid amide; N-[(2-Fluorophenyl)methyl]-4-oxo-4-(1-phenyl-3,4-dihydro-1H-isoquinolin-2-yl) butyric acid amide; N-[(4-Fluorophenyl)methyl]-4-oxo-4-(1-phenyl-3,4-dihydro-1H-isoquinolin-2-yl) butyric acid amide; N-[(2,5-Difluorophenyl)methyl]-4-oxo-4-(1-phenyl-3,4-dihydro-1H-isoquinolin-2-yl) butyric acid amide; N-(1-Naphthylmethyl)-4-oxo-4-(1-phenyl-3,4-dihydro-1H-isoquinolin-2-yl) butyric acid amide; 4-(7-Methyl-1-phenyl-3,4-dihydro-1H-isoquinolin-2-yl)-4-oxo-N-propylbutyric acid amide; 4-(5-Methyl-1-phenyl-3,4-dihydro-1H-isoquinolin-2-yl)-4-oxo-N-propylbutyric acid amide; 4-(5,7-Dimethyl-1-phenyl-3,4-dihydro-1H-isoquinolin-2-yl)-4-oxo-N-propylbutyric acid amide; 4-Oxo-4-(1-phenyl-3,4-dihydro-1H-isoquinolin-2-yl)-N-propylbutyric acid amide; 4-Oxo-4-(1-(2-tolyl)-3,4-dihydro-1H-isoquinolin-2-yl)-N-[[3-(trifluoromethyl)-phenyl]methyl]butyric acid amide; 4-Oxo-4-(1-(2-tolyl)-3,4-dihydro-1H-isoquinolin-2-yl)-N-[[4-(trifluoromethyl)-phenyl]methyl]butyric acid amide; 4-Oxo-4-(1-(2-tolyl)-3,4-dihydro-1H-isoquinolin-2-yl)-N-[[2-(trifluoromethyl)-phenyl]methyl]butyric acid amide; 4-Oxo-4-(1-(2-tolyl)-6-methyl-3,4-dihydro-1H-isoquinolin-2-yl)-N-[[3-(trifluoromethyl)-phenyl]methyl]butyric acid amide; 4-(1-(2-Methyl-prop-2-yl)-3,4-dihydro-1H-isoquinolin-2-yl)-4-oxo-N-[[3-(trifluoromethyl)phenyl]methyl]butyric acid amide; 4-(1-Cyclohexyl-3,4-dihydro-1H-isoquinolin-2-yl)-4-oxo-N-[[3-(trifluoromethyl)-phenyl]methyl]butyric acid amide; and 4-Oxo-4-(1-(2-fluorophenyl)-3,4-dihydro-1H-isoquinolin-2-yl)-N-[[3-(trifluoromethyl)phenyl]methyl]butyric acid amide.

### Formula 32

In an embodiment, the Kv7 channel activator may be selected from one of the following compounds according to Formula 32. Such compounds are described in International Publication No. WO2010037863A1, published Apr. 8, 2010, and corresponding to International Application No. PCT/EP2009/062859 filed Oct. 2, 2009; US Publication No. US20120238547A1, published Sep. 20, 2012 and corresponding to U.S. application Ser. No. 13/394,345 filed Sep. 2, 2010; which are incorporated by reference in their entirety herein. In the case of any conflict of terminology in the context of Formula 32, these references incorporated by reference herein control.

In an embodiment, the Kv7 channel activator is a compound according to formula 32:

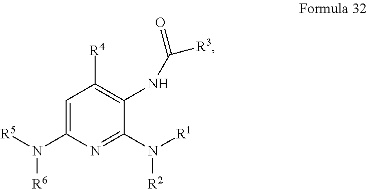

a stereoisomer or a mixture of its stereoisomers, or a pharmaceutically acceptable addition salt thereof, or an N-oxide thereof, wherein R¹ and R², together with the nitrogen to which they are attached, form a heterocyclic ring selected from pyrrolidinyl, 2,5-dihydro-1H-pyrrol-1-yl, azetidinyl, thiazolidinyl, piperidinyl, piperazinyl and morpholinyl, which pyrrolidinyl, azetidinyl, piperidinyl, piperazinyl and morpholinyl is optionally substituted one or more times with a substituent selected from the group consisting of halo and trifluoromethyl; R³ represents a heterocyclic ring selected from furanyl and pyrrolyl which furanyl and pyrrolyl is optionally substituted one or more times with substituents selected from C₁₋₆-alkyl, C₁₋₆-alkoxy, halo and trifluoromethyl; R⁴ represents C₁₋₆-alkyl; and R⁵ and R⁶, together with the nitrogen to which they are attached, form a heterocyclic ring selected from morpholinyl and 1,4-oxazepanyl.

In further embodiments, R¹ and R² together with the nitrogen to which they are attached is pyrrolidinyl which is optionally substituted one or more times with halo.

In further embodiments, R³ represents furanyl which is optionally substituted with C₁₋₆-alkyl.

In further embodiments, R⁴ represents C₁₋₆-alkyl.

In further embodiments, R⁵ and R⁶, together with the nitrogen to which they are attached, represents morpholinyl.

In further embodiments, the compound is 3-Methyl-furan-2-carboxylic acid [2-((R)-3-fluoro-pyrrolidin-1-yl)-4-methyl-6-[1,4]oxazepan-4-yl-pyridin-3-yl]-amide.

In further embodiments, the compound is 3-Methyl-furan-2-carboxylic acid [2-((R)-3-fluoro-pyrrolidin-1-yl)-4-methyl-6-morpholin-4-yl-pyridin-3-yl]-amide;

### Formula 33

In an embodiment, the Kv7 channel activator may be selected from one of the following compounds according to Formula 33. Such compounds are described in US Publication No. US20120232058A1, published Sep. 13, 2012, and corresponding to U.S. application Ser. No. 13/394,468 filed Sep. 2, 2010; International Publication No. WO2011026891A1, published Mar. 10, 2011 and corresponding to International Application No. PCT/EP2010/062860 filed Sep. 2, 2010; which are incorporated by reference in their entirety herein. In the case of any conflict of terminology in the context of Formula 33, these references incorporated by reference herein control.

In an embodiment, the Kv7 channel activator is a compound according to formula 33:

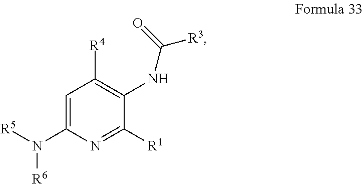

a stereoisomer or a mixture of its stereoisomers, or a pharmaceutically-acceptable addition salt thereof, or an N-oxide thereof, wherein R¹ represents C₁₋₆-alkyl; R³ represents a heterocyclic ring selected from furanyl and pyrrolyl which furanyl and pyrrolyl is optionally substituted one or more times with substituents selected from C₁₋₆-alkyl, C₁₋₆-alkoxy, halo and trifluoromethyl; R⁴ represents C₁₋₆-alkyl; and R⁵ and R⁶, together with the nitrogen to which they are attached, form a heterocyclic ring selected from morpholinyl and 1,4-oxazepanyl.

In further embodiments, R¹ represents methyl or iso-propyl.

In further embodiments, R³ represents furanyl which is optionally substituted with C₁₋₆-alkyl.

In further embodiments, R⁴ represents C₁₋₆-alkyl.

In further embodiments, R⁵ and R⁶, together with the nitrogen to which they are attached, represents 1,4-oxazepanyl.

In further embodiments, the compound is 3-Methyl-furan-2-carboxylic acid (2-isopropyl-4-methyl-6-[1,4]oxazepan-4-yl-pyridin-3-yl)-amide; or 3-Methyl-furan-2-carboxylic acid (2,4-dimethyl-6-[1,4]oxazepan-4-yl-pyridin-3-yl)-amide; or a stereoisomer or a mixture of its stereoisomers, or a pharmaceutically-acceptable addition salt thereof, or an N-oxide thereof.

### Formula 34

In an embodiment, the Kv7 channel activator may be selected from one of the following compounds according to Formula 34. Such compounds are described in International Publication No. WO2010122064A1, published Oct. 28, 2010, and corresponding to International Application No. PCT/EP2010/055284 filed Apr. 21, 2010; US Publication No. US20120115900A1, published May 10, 2012 and corresponding to U.S. application Ser. No. 13/265,273 filed Apr. 21, 2010; which are incorporated by reference in their entirety herein. In the case of any conflict of terminology in the context of Formula 34, these references incorporated by reference herein control.

In an embodiment, the Kv7 channel activator is a compound according to formula 34:

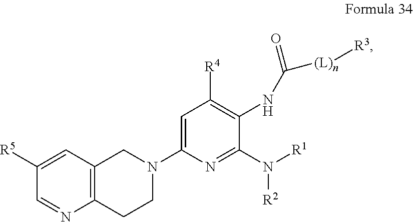

a stereoisomer or a mixture of its stereoisomers, or a pharmaceutically-acceptable addition salt thereof, or an N-oxide thereof, wherein R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halo, hydroxy, amino, C₁₋₆-alkyl, trifluoromethyl, C₁₋₆-alkoxy, hydroxy-C₁₋₆-alkyl and C₁₋₆-alkoxy-C₁-C₆-alkyl; L represents a linker selected from —CR′R″—, —CH₂—CR′R″—, —CR′R″—CH₂—, and —O—, wherein R′ and R″, independently of each other, represent hydrogen, C₁₋₆-alkyl or halo; n is 0 or 1; R³ represents C₁₋₆-alkyl, phenyl, pyridyl, furanyl, imidazolyl, oxazolyl, isoxazolyl, thiazolyl, isothiazolyl, or C₃₋₆-cycloalkyl, which phenyl, pyridyl, furanyl, imidazolyl, oxazolyl, isoxazolyl, thiazolyl and isothiazolyl is optionally substituted one or more times with substituents selected from C₁₋₆-alkyl, C₃₋₆-cycloalkyl, phenyl, C₁₋₆-alkoxy, halo and trifluoromethyl; R⁴ represents hydrogen, halo or C₁₋₆-alkyl; and R⁵ represents hydrogen or halo.

In further embodiments, R¹ and R² together with the nitrogen to which they are attached is pyrrolidinyl or piperidinyl, which pyrrolidinyl and piperidinyl is optionally substituted one or more times with halo.

In further embodiments, R¹ and R² together with the nitrogen to which they are attached is pyrrolidinyl, which pyrrolidinyl is optionally substituted one or more times with halo.

In further embodiments, L represents —CH₂-.

In further embodiments, R³ represents C₁₋₆-alkyl.

In further embodiments, R³ represents phenyl, pyridyl, furanyl, imidazolyl, oxazolyl, isoxazolyl, thiazolyl, isothiazolyl, or C₃₋₆-cycloalkyl, which phenyl, pyridyl, furanyl, imidazolyl, oxazolyl, isoxazolyl, thiazolyl and isothiazolyl is optionally substituted one or more times with substituents selected from C₁₋₆-alkyl and halo.

In further embodiments, R⁴ represents C₁₋₆-alkyl.

In further embodiments, R⁵ represents hydrogen.

In further embodiments, the Kv7 channel activator is selected from the group consisting of: 2-(3,5-Difluoro-phenyl)-N-[6-(7,8-dihydro-5H-[1,6]naphthyridin-6-yl)-4-methyl-2-pyrrolidin-1-yl-pyridin-3-yl]-acetamide N-[6-(7,8-Dihydro-5H-[1,6]naphthyridin-6-yl)-4-methyl-2-pyrrolidin-1-yl-pyridin-3-yl]-3,3-dimethyl-butyramide; N-[6-(7,8-Dihydro-5H-[1,6]naphthyridin-6-yl)-4-methyl-2-pyrrolidin-1-yl-pyridin-3-yl]-3-fluoro-benzamide; N-[6-(7,8-dihydro-5H-1,6-naphthyridin-6-yl)-2-[(3R)-3-fluoropyrrolidin-1-yl]-4-methyl-3-pyridyl]-3-methyl-furan-2-carboxamide; 2-(3,5-difluorophenyl)-N-[6-(7,8-dihydro-5H-1,6-naphthyridin-6-yl)-2-[(3R)-3-fluoropyrrolidin-1-yl]-4-methyl-3-pyridyl]acetamide; N-[2-(4,4-difluoro-1-piperidyl)-6-(7,8-dihydro-5H-1,6-naphthyridin-6-yl)-4-methyl-3-pyridyl]-3-methyl-furan-2-carboxamide; 2-(3,5-difluorophenyl)-N-[2-(4,4-difluoro-1-piperidyl)-6-(7,8-dihydro-5H-1,6-naphthyridin-6-yl)-4-methyl-3-pyridyl]acetamide; N-[2-(4,4-difluoro-1-piperidyl)-6-(7,8-dihydro-5H-1,6-naphthyridin-6-yl)-4-methyl-3-pyridyl]-1-methyl-imidazole-2-carboxamide; N-[2-(4,4-difluoro-1-piperidyl)-6-(7,8-dihydro-5H-1,6-naphthyridin-6-yl)-4-methyl-3-pyridyl]-5-methyl-oxazole-4-carboxamide; N-[2-(4,4-difluoro-1-piperidyl)-6-(7,8-dihydro-5H-1,6-naphthyridin-6-yl)-4-methyl-3-pyridyl]-4-methyl-thiazole-5-carboxamide; N-[2-(4,4-difluoro-1-piperidyl)-6-(7,8-dihydro-5H-1,6-naphthyridin-6-yl)-4-methyl-3-pyridyl]-3-methyl-isoxazole-4-carboxamide; N-[2-(4,4-difluoro-1-piperidyl)-6-(7,8-dihydro-5H-1,6-naphthyridin-6-yl)-4-methyl-3-pyridyl]-4-methyl-oxazole-5-carboxamide; 2-cyclopropyl-N-[2-(4,4-difluoro-1-pipridyl)-6-(7,8-dihydro-5H-1,6-naphthyridin-6-yl)-4-methyl-3-pyridyl]acetamide; N-[2-(4,4-difluoro-1-piperidyl)-6-(7,8-dihydro-5H-1,6-naphthyridin-6-yl)-4-methyl-3-pyridyl]-5-ethyl-oxazole-4-carboxamide; N-[2-(4,4-difluoro-1-piperidyl)-6-(7,8-dihydro-5H-1,6-naphthyridin-6-yl)-4-methyl-3-pyridyl]-3-methyl-pyridine-2-carboxamide; N-[2-(4,4-difluoro-1-piperidyl)-6-(7,8-dihydro-5H-1,6-naphthyridin-6-yl)-4-methyl-3-pyridyl]-2,5-dimethyl-oxazole-4-carboxamide; N-[2-(4,4-difluoro-1-piperidyl)-6-(7,8-dihydro-5H-1,6-naphthyridin-6-yl)-4-methyl-3-pyridyl]-5-phenyl-oxazole-4-carboxamide; 5-cyclopropyl-N-[2-(4,4-difluoro-1-piperidyl)-6-(7,8-dihydro-5H-1,6-naphthyridin-6-yl)-4-methyl-3-pyridyl]oxazole-4-carb oxamide; N-[2-(4,4-difluoro-1-piperidyl)-6-(7,8-dihydro-5H-1,6-naphthyridin-6-yl)-4-methyl-3-pyridyl]-5-methyl-isothiazole-4-carboxamide; ethyl N-[2-(4,4-difluoro-1-piperidyl)-6-(7,8-dihydro-5H-1,6-naphthyridin-6-yl)-4-methyl-3-pyridyl]carbamate; N-[6-(3-chloro-7,8-dihydro-5H-1,6-naphthyridin-6-yl)-2-[(3R)-3-fluoropyrrolidin-1-yl]-4,6-dimethyl-1H-pyridin-3-yl]-3,3-dimethyl-butanamide; or a stereoisomer or a mixture of its stereoisomers, or a pharmaceutically-acceptable addition salt thereof, or an N-oxide thereof.

### Formula 35

In an embodiment, the Kv7 channel activator may be selected from one of the following compounds according to Formula 35. Such compounds are described in US Publication No. US20120059037A1, published Mar. 8, 2012, and corresponding to U.S. application Ser. No. 13/256,893 filed Mar. 11, 2010; International Publication No. WO2010105960A1, published Sep. 23, 2010 and corresponding to International Application No. PCT/EP2010/053072 filed Mar. 11, 2010; which are incorporated by reference in their entirety herein. In the case of any conflict of terminology in the context of Formula 35, these references incorporated by reference herein control.

In an embodiment, the Kv7 channel activator is a compound according to formula 35:

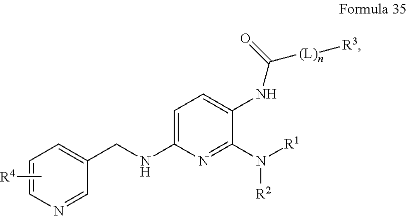

a stereoisomer or a mixture of its stereoisomers, or a pharmaceutically acceptable addition salt thereof, or an N-oxide thereof, wherein R¹ and R², independently of each other, represent hydrogen, C₁₋₆-alkyl, hydroxy-C₁₋₆-alkyl or C₁₋₆-alkoxy-C₁₋₆-alkyl; or R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halogen, hydroxy, amino, C₁₋₆-alkyl, trifluoromethyl, C₁₋₆-alkoxy, hydroxy-C₁₋₆-alkyl and C₁₋₆-alkoxy-C₁₋₆-alkyl; L represents a linker selected from —CR′R″—, —CH₂—CR′R″— and -CR′R″—CH₂—, wherein R′ and R″, independently of each other, represent hydrogen, C₁₋₆-alkyl or halogen; n is 0, 1; R³ represents C₁₋₆-alkyl, phenyl or furanyl, which phenyl and furanyl is optionally substituted one or more times with substituents selected from C₁₋₆-alkyl, C₁₋₆-alkoxy, halogen and trifluoromethyl; and R⁴ represents hydrogen, halogen or C₁₋₆-alkyl.

In further embodiments, R¹ and R² together with the nitrogen to which they are attached is pyrrolidinyl, which pyrrolidinyl is optionally substituted one or more times with halogen.

In further embodiments, L represents —CH₂—, and n is 1.

In further embodiments, R³ represents phenyl, which phenyl is optionally substituted one or more times with halogen.

In further embodiments, R³ represents C₁₋₆-alkyl.

In further embodiments, R⁴ represents halogen.

In further embodiments, R⁴ represents hydrogen.

In further embodiments, the Kv7 Channel activator is selected from the group consisting of: N-{6-[(6-Chloro-pyridin-3-ylmethyl)-amino]-2-pyrrolidin-1-yl-pyridin-3-yl}-2-(3,5-difluoro-phenyl)-acetamide; 2-(3,5-Difluoro-phenyl)-N-{6-[(pyridin-3-ylmethyl)-amino]-2-pyrrolidin-1-yl-pyridin-3-yl}-acetamide; N-{6-[(6-Chloro-pyridin-3-ylmethyl)-amino]-2-pyrrolidin-1yl-pyridin-3-yl}-3,3-dimethyl-butyramide; or a stereoisomer or a mixture of its stereoisomers, or a pharmaceutically acceptable addition salt thereof, or an N-oxide thereof.

### Formula 36

In an embodiment, the Kv7 channel activator may be selected from one of the following compounds according to Formula 36. Such compounds are described in Patent Cooperation Treaty application No. EP2010/052257 published Sep. 2, 2010, which is incorporated by reference in its entirety herein. In the case of any conflict of terminology in the context of Formula 36, these references incorporated by reference herein control.

In an embodiment, the Kv7 channel activator is a compound according to formula 36:

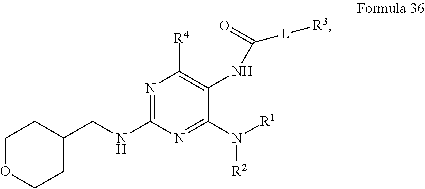

a stereoisomer or a mixture of its stereoisomers, or a pharmaceutically-acceptable addition salt thereof, or an N-oxide thereof, wherein R¹ and R², independently of each other, represent hydrogen, Ci₋₆-alkyl, hydroxy-C-1-6-alkyl or Ci₋₆-alkoxy-Ci₋₆-alkyl; or R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halogen, hydroxy, amino, Ci₋₆-alkyl, trifluoro-methyl, Ci₋₆-alkoxy, hydroxy-d-₆-alkyl and d-₆-alkoxy-d-₆-alkyl; L represents a linker selected from —CR¹R″—, —CH₂—CR¹R″— and —CR′R″—CH₂—, wherein R′ and R″, independently of each other, represent hydrogen, d-₆-alkyl or halogen; R³ represents Ci-6-alkyl or phenyl, which phenyl is optionally substituted one or more times with substituents selected from C₁₋₆-alkyl, Ci-6-alkoxy, halogen and trifluoromethyl; and R⁴ represents H or Ci₋₆-alkyl.

In further embodiments, R¹ and R², independently of each other, represent Ci₋₆-alkyl.

In further embodiments, R¹ and R² together with the nitrogen to which they are attached, is morpholinyl.

In further embodiments, L represents —CH₂-.

In further embodiments, R³ represents phenyl, which phenyl is optionally substituted one or more times with halogen.

In further embodiments, R⁴ represents methyl.

In further embodiments, the Kv7 Channel activator is selected from the group consisting of: 2-(3,5-Difluoro-phenyl)-N-{4-methyl-6-morpholin-4-yl-2-[(tetrahydro-pyran-4-ylmethyl)-amino]-pyrimidin-5-yl}-acetamide; 2-(3,5-Difluoro-phenyl)-N-{4-dimethylamino-6-methyl-2-[(tetrahydro-pyran-4-ylmethyl)-amino]-pyrimidin-5-yl}-acetamide; or a stereoisomer or a mixture of its stereoisomers, or a pharmaceutically-acceptable addition salt thereof, or an/V-oxide thereof.

### Formula 37

In an embodiment, the Kv7 channel activator may be selected from one of the following compounds according to Formula 37. Such compounds are described in International Publication No. WO2010094644A1, published Aug. 26, 2010, and corresponding to International Application No. PCT/EP2010/051839 filed Feb. 15, 2010; which is incorporated by reference in its entirety herein. In the case of any conflict of terminology in the context of Formula 37, the reference incorporated by reference herein controls.

In an embodiment, the Kv7 channel activator is a compound according to formula 37:

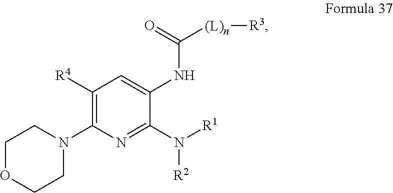

a stereoisomer or a mixture of its stereoisomers, or a pharmaceutically-acceptable addition salt thereof, or an N-oxide thereof, wherein R¹ and R², independently of each other, represent Ci₋₆-alkyl; or R¹ and R², together with the nitrogen to which they are attached, form a heterocyclic ring selected from pyrrolidinyl, 2,5-dihydro-1H-pyrrol-1-yl, piperidinyl and morpholinyl, which pyrrolidinyl and piperidinyl is optionally substituted one or more times with a substituent selected from the group consisting of halogen, hydroxy, amino, C₁₋₆-alkyl, trifluoromethyl, Ci₋₆-alkoxy, hydroxy-d-₆-alkyl and Ci₋₆-alkoxy-Ci₋₆-alkyl; L represents a linker selected from —CR¹R″— and —O—CR′R″—, wherein R′ and R″, independently of each other, represent hydrogen, d-₆-alkyl or halogen; n is 0 or 1; R³ represents d-₆-alkyl or phenyl, which phenyl is optionally substituted one or more times with substituents selected from d-6-alkyl, Ci-6-alkoxy, halogen and trifluoromethyl; and R⁴ represents Ci-6-alkyl.

In further embodiments, R¹ and R² together with the nitrogen to which they are attached is pyrrolidinyl, which pyrrolidinyl is optionally substituted one or more times with halogen.

In further embodiments, R′ and R″, independently of each other, represents hydrogen or methyl.

In further embodiments, R³ represents phenyl, which phenyl is optionally substituted one or more times with halogen.

In further embodiments, R³ represents d-e-alkyl.

In further embodiments, R⁴ represents methyl.

In further embodiments, the Kv7 Channel activator is selected from the group consisting of: 3,3-Dimethyl-N-(5-methyl-6-morpholin-4-yl-2-pyrrolidin-1-yl-pyridin-3-yl)-butyramide; 2-(3,5-Difluoro-phenyl)-N-(5-methyl-6-morpholin-4-yl-2-pyrrolidin-1-yl-pyridin-3-yl)-acetamide; N-[2-((R)-3-Fluoro-pyrrolidin-1-yl)-5-methyl-6-morpholin-4-yl-pyridin-3-yl]-3,3-dimethylbutyramide; (S)—N-[2-((R)-3-Fluoro-pyrrolidin-1-yl)-5-methyl-6-morpholin-4-yl-pyridin-3-yl]-2-phenylpropionamide; [2-((R)-3-Fluoro-pyrrolidin-1-yl)-5-methyl-6-morpholin-4-yl-pyridin-3-yl]-carbamic acid ethyl ester; (S)—N-[2-((R)-3-Fluoro-pyrrolidin-1-yl)-5-methyl-6-morpholin-4-yl-pyridin-3-yl]-2-methyl-butyramide; or a stereoisomer or a mixture of its stereoisomers, or a pharmaceutically-acceptable addition salt thereof, or an/V-oxide thereof.

### Formula 38

In an embodiment, the Kv7 channel activator may be selected from one of the following compounds according to Formula 38. Such compounds are described in International Publication No. WO2010094645A1, published Aug. 26, 2010, and corresponding to International Application No. PCT/EP2010/051840 filed Feb. 15, 2010; which is incorporated by reference in its entirety herein. In the case of any conflict of terminology in the context of Formula 38, the reference incorporated by reference herein controls.

In an embodiment, the Kv7 channel activator is a compound according to Formula 38:

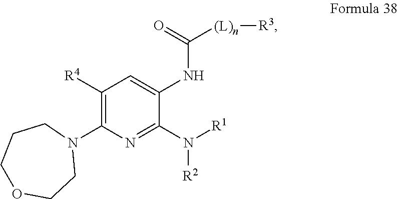

a stereoisomer or a mixture of its stereoisomers, or a pharmaceutically-acceptable addition salt thereof, or an N-oxide thereof, wherein R¹ and R², independently of each other, represent d-e-alkyl; or R¹ and R², together with the nitrogen to which they are attached, form a heterocyclic ring selected from pyrrolidinyl, 2,5-dihydro-1H-pyrrol-1-yl, piperidinyl, mor-pholinyl and 1,4-oxazepanyl, which pyrrolidinyl and piperidinyl is optionally substituted one or more times with a substituent selected from the group consisting of halogen, hydroxy, amino, d-₆-alkyl, trifluoromethyl, d-₆-alkoxy, hydroxy-Ci₋₆-alkyl and Ci₋₆-alkoxy-Ci₋₆-alkyl; L represents a linker selected from —CR¹R″— and —O—CR′R″—, wherein R′ and R″, independently of each other, represent hydrogen, Ci₋₆-alkyl or halogen; n is 0 or 1; R³ represents C₁₋₆-alkyl, phenyl or furanyl, which phenyl and furanyl is optionally substituted one or more times with substituents selected from d-₆-alkyl, Ci₋₆-alkoxy, halogen and trifluoromethyl; and R⁴ represents d-₆-alkyl.

In further embodiments, R¹ and R² together with the nitrogen to which they are attached is pyrrolidinyl, which pyrrolidinyl is optionally substituted one or more times with halogen.

In further embodiments, R¹ and R² together with the nitrogen to which they are attached is 1,4-oxazepanyl.

In further embodiments, L is —CH₂— and n is 1.

In further embodiments, n is 0.

In further embodiments, R³ represents phenyl, which phenyl is optionally substituted one or more times with halogen.

In further embodiments, the Kv7 Channel activator is selected from the group consisting of: 2-(3,4-Difluoro-phenyl)-N-[2-((R)-3-fluoro-pyrrolidin-1-yl)-5-methyl-6-[1, 4]oxazepan-4-yl-pyridin-3-yl]-acetamide; 2-(3,5-Difluoro-phenyl)-/V-(5-methyl-2,6-bis-[1, 4]oxazepan-4-yl-pyridin-3-yl)-acetamide; 3-Fluoro-/V-(5-methyl-6-[1,4]oxazepan-4-yl-2-pyrrolidin-1-yl-pyridin-3-yl)-benzamide; 3-methyl-N-[5-methyl-6-(1,4-oxazepan-4-yl)-2-pyrrolidin-1-yl-3-pyridyl]furan-2-carboxamide; 2-(3-fluorophenyl)-N-[2-[(3R)-3-fluoropyrrolidin-1-yl]-5-methyl-6-(1,4-oxazepan-4-yl)-3-pyridyl]acetamide; or a stereoisomer or a mixture of its stereoisomers, or a pharmaceutically-acceptable addition salt thereof, or an/V-oxide thereof.

### Formula 39

In an embodiment, the Kv7 channel activator may be selected from one of the following compounds according to Formula 1. Such compounds are described in International Publication No. WO2008142140A2, published Nov. 27, 2008, and corresponding to International Application No. PCT/EP2008/056322 filed May 22, 2008; U.S. Pat. No. 8,178,544 published May 15, 2012 and corresponding to U.S. application Ser. No. 12/601,124 filed May 22, 2008; which are incorporated by reference in their entirety herein. In the case of any conflict of terminology in the context of Formula 39, the reference incorporated by reference herein controls.

In an embodiment, the Kv7 channel activator is a compound according to Formula 39:

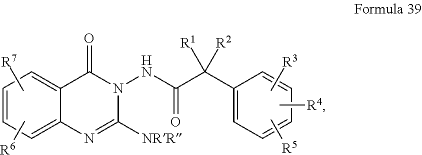

a stereoisomer or a mixture of its stereoisomers, or a pharmaceutically-acceptable addition salt thereof, or an N-oxide thereof, wherein R¹ represents hydrogen, alkyl or halo; and R² represents hydrogen, alkyl, cycloalkyl, halo, haloalkyl, hydroxyalkyl, hydroxy, alkoxy, phenyl, phenyl-alkyl, amino, alkyl-carbonyl-amino, cyano or nitro; or R¹ and R² together with the carbon atom to which they are attached form a cycloalkyl (spiro) group; or R¹ represents hydrogen; and R² together with one of R³, R⁴ and R⁵, attached in ortho-position on the aromatic ring, form a —(CH₂)ₙ— bridge, wherein n is 1, 2 or 3; R³, R⁴ and R⁵, independently of each other, represent hydrogen, alkyl, cycloalkyl, halo, haloalkyl, hydroxy, alkoxy, haloalkoxy, amino, alkyl-carbonyl-amino, alkyl-sulfanyl, alkyl-sulfonyl, phenyl, phenoxy, benzoyl, cyano or nitro; or two of R³, R⁴ and R⁵, together form a methylenedioxy group; and the remaining of R³, R⁴ and R⁵ is as defined above; or two of R³, R⁴ and R⁵, together with the phenyl ring to which they are attached, form a naphthyl group; and the remaining of R³,
R⁴ and R⁵ is as defined above; or one of R³, R⁴ and R⁵, attached in ortho-position on the aromatic ring, and together with R² form a —(CH₂)ₙ— bridge, wherein n is 1, 2 or 3; and the remaining of R³, R⁴ and R⁵, are as defined above; R⁶ and R⁷, independently of each other, represent hydrogen, alkyl, cycloalkyl, halo, haloalkyl, hydroxy, alkoxy, haloalkoxy, amino, alkyl-carbonyl-amino, nitro, cyano or phenyl; and R′ and R″, independently of each other, represent alkyl, hydroxy-alkyl, amino-alkyl, cycloalkyl, phenyl-alkyl, phenyl-hydroxyalkyl, N-alkyl-amino-alkyl, N,N-dialkyl-amino-alkyl, alkoxy-alkyl, piperidinyl, N-alkyl-piperidinyl, furanyl-alkyl, pyridinyl-alkyl, pyrazolyl-alkyl, imidazolyl-alkyl, pyrimidinyl, pyrimidinyl substituted with one or two substituents selected from N-alkyl-amino, N,N-dialkyl-amino and phenyl; or R′ and R″, together with the nitrogen to which they are attached, form a heterocyclic ring selected from pyrrolidinyl, 2,5-dihydro-1H-pyrrol-1-yl, thiazolidinyl, piperidinyl, piperazinyl, homopiperazinyl, morpholinyl and homomorpholinyl, which pyrrolidinyl, piperidinyl, piperazinyl, homopiperazinyl, morpholinyl and homomorpholinyl is optionally substituted one or more times with a substituent selected from the group consisting of alkyl, alkoxy, alkoxy-alkyl, alkyl-carbonyl-amino, cycloalkyl-carbonyl-amino, hydroxy-alkyl, hydroxy, amino, N-alkyl-amino, N,N-dialkyl-amino, amino-alkyl, N-alkyl-amino-alkyl, N,N-dialkyl-amino-alkyl, carbamoyl-alkyl, N-alkyl-carbamoyl-alkyl, N,N-dialkyl-carbamoyl-alkyl, N-hydroxy-alkyl-carbamoyl, N,N-dialkyl-amino-alkyl-carbamoyl, alkoxy-carbonyl, cyano-alkyl, pyrrolidinyl, pyrrolidinyl-alkyl, piperidinyl, piperidinyl-carbonyl, hydroxy-piperidinyl, hydroxy-piperidinyl-alkyl, hydroxy-piperidinyl-carbonyl, N-alkyl-piperidinyl, piperidinyl-alkyl, N-alkyl-piperidinyl-alkyl, morpholino-alkyl, morpholino-alkyl-carbamoyl, morpholino-carbonyl-alkyl, triazolyl-alkyl, piperazinyl, piperazinyl-alkyl, piperazinyl-carbonyl, N-alkyl-piperazinyl, N-alkyl-piperazinyl-alkyl, N-alkyl-piperazinyl-carbonyl, pyridinyl, pyridinyl-alkyl, and pyridinyl substituted once or twice with alkyl, trifluoromethyl and/or cyano.

In further embodiments, R¹ represents hydrogen or alkyl; and R² represents hydrogen or alkyl.

In further embodiments, R³, R⁴ and R⁵, independently of each other, represent hydrogen, alkyl, halo, haloalkyl, hydroxy, alkoxy, alkyl-sulfanyl, alkyl-sulfonyl, phenyl or phenoxy.

In further embodiments, R⁶ and R⁷, independently of each other, represent hydrogen, halo, haloalkyl, hydroxy, alkoxy, or amino.

In further embodiments, R′ and R″, independently of each other, represent alkyl, hydroxy-alkyl, cycloalkyl, or phenyl-alkyl.

In further embodiments, R′ and R″, together with the nitrogen to which they are attached, form a heterocyclic ring selected from pyrrolidinyl, thiazolidinyl, piperidinyl, piperazinyl, homopiperazinyl and morpholinyl, which piperazinyl is optionally substituted one or more times with alkyl.

In further embodiments, the Kv7 Channel activator is selected from the group consisting of: 2-(3,5-Difluoro-phenyl)-N-(4-oxo-2-pyrrolidin-1-yl-4H-quinazolin-3-yl)-acetamide; 2-(3,5-Difluoro-phenyl)-N-(2-dimethylamino-4-oxo-4H-quinazolin-3-yl)-acetamide; 2-(4-Chloro-phenyl)-N-(2-diethylamino-4-oxo-4H-quinazolin-3-yl)-acetamide; 2-(3,5-Difluoro-phenyl)-N-[2-(ethyl-methyl-amino)-4-oxo-4H-quinazolin-3-yl]-acetamide; 2-(3,5-Difluoro-phenyl)-N-(2-dimethylamino-4-oxo-7-trifluoromethyl-4H-quinazolin-3-yl)-propionamide; N-(2-Diethylamino-4-oxo-4H-quinazolin-3-yl)-2-(4-methanesulfonyl-phenyl)-acetamide; N-{2-[(2-Methoxy-ethyl)-methyl-amino]-4-oxo-4H-quinazolin-3-yl}-2-(4-trifluoromethyl-phenyl)-acetamide; 2-(4-Butoxy-phenyl)-N-{2-[(2-methoxy-ethyl)-methyl-amino]-4-oxo-4H-quinazolin-3-yl}-acetamide; N-[2-(Benzyl-methyl-amino)-4-oxo-4H-quinazolin-3-yl]-2-(4-methanesulfonyl-phenyl)-acetamide; 2-(4-Butoxy-phenyl)-N-(4-oxo-2-pyrrolidin-1-yl-4H-quinazolin-3-yl)-acetamide; 2-(3,5-Difluoro-phenyl)-N-(4-oxo-2-thiazolidin-3-yl-4H-quinazolin-3-yl)-acetamide; N-(2-Diethylamino-4-oxo-4H-quinazolin-3-yl)-2-(3,5-difluoro-phenyl)-acetamide; 2-(3,5-Bis-trifluoromethyl-phenyl)-N-(4-oxo-2-thiazolidin-3-yl-4H-quinazolin-3-yl)-acetamide; 2-Benzo[1,3]dioxol-5-yl-N-(2-diethylamino-4-oxo-4H-quinazolin-3-yl)-acetamide; N-(2-Diethylamino-4-oxo-4H-quinazolin-3-yl)-2-phenyl-acetamide; or N-[2-(Ethyl-methyl-amino)-4-oxo-4H-quinazolin-3-yl]-2-phenyl-acetamide; or a stereoisomer or a mixture of its stereoisomers, a pharmaceutically-acceptable addition salt thereof.

In further embodiments, the Kv7 Channel activator is selected from the group consisting of: 2-(3-Fluoro-4-trifluoromethyl-phenyl)-N-(4-oxo-2-pyrrolidin-1-yl-4H-quinazolin-3-yl)-acetamide; 2-(3,5-Bis-trifluoromethyl-phenyl)-N-{2-[(2-methoxy-ethyl)-methyl-amino]-4-oxo-4H-quinazolin-3-yl}-acetamide; N-[2-(Benzyl-methyl-amino)-4-oxo-4H-quinazolin-3-yl]-2-(3,4-difluoro-phenyl)-acetamide; 2-(3,5-Difluoro-phenyl)-N-(2-morpholin-4-yl-4-oxo-4H-quinazolin-3-yl)-acetamide; 2-(4-Chloro-phenyl)-N-{7-fluoro-2-[(2-methoxy-ethyl)-methyl-amino]-4-oxo-4H-quinazolin-3-yl}-acetamide; 2-(4-Chloro-phenyl)-N-(2-morpholin-4-yl-4-oxo-4H-quinazolin-3-yl)-acetamide; 2-(3,5-Difluoro-phenyl)-N-{7-fluoro-2-[(2-methoxy-ethyl)-methyl-amino]-4-oxo-4H-quinazolin-3-yl}-acetamide; 2-(4-Chloro-phenyl)-N-(2-dimethylamino-4-oxo-4H-quinazolin-3-yl)-acetamide; N-(2-Diethylamino-6-fluoro-4-oxo-4H-quinazolin-3-yl)-2-(3,5-difluoro-phenyl)-acetamide; 2-(4-Chloro-phenyl)-N-(2-diethylamino-6-fluoro-4-oxo-4H-quinazolin-3-yl)-acetamide; 2-(3,5-Difluoro-phenyl)-N-[2-(4-methyl-piperazin-1-yl)-4oxo-4H-quinazolin-3-yl]-acetamide; N-(2-Diethylamino-6-fluoro-4-oxo-4H-quinazolin-3-yl)-2-(3-fluoro-5-trifluoromethyl-phenyl)-acetamide; N-[2-(Benzyl-methyl-amino)-4-oxo-4H-quinazolin-3-yl]-2-(3,5-difluoro-phenyl)-acetamide; 2-(3,5-Difluoro-phenyl)-N-{2-[(2-methoxy-ethyl)-methyl-amino]-4-oxo-4H-quinazolin-3-yl}-acetamide; 2-(3-Fluoro-5-trifluoromethyl-phenyl)-N-(4-oxo-2-pyrrolidin-1-yl-7-trifluoromethyl-4H-quinazolin-3-yl)-acetamide; 2-(3,5-Difluoro-phenyl)-N-(4-oxo-2-pyrrolidin-1-yl-7-trifluoromethyl-4H-quinazolin-3-yl)-acetamide; 2-(3-Fluoro-5-trifluoromethyl-phenyl)-N-{2-[(2-methoxy-ethyl)-methyl-amino]-4-oxo-7-trifluoromethyl-4H-quinazolin-3-yl}-acetamide; 2-(3,5-Difluoro-phenyl)-N-{2-[(2-methoxy-ethyl)-methyl-amino]-4-oxo-7-trifluoromethyl-4H-quinazolin-3-yl}-acetamide; N-(6-Butoxy-2-morpholin-4-yl-4-oxo-4H-quinazolin-3-yl)-2-(3,5-difluoro-phenyl)-acetamide; N-(5-Chloro-4-oxo-2-pyrrolidin-1-yl-4H-quinazolin-3-yl)-2-(3,5-difluoro-phenyl)-acetamide; N-(5-Chloro-2-diethylamino-4-oxo-4H-quinazolin-3-yl)-2-(3,5-difluoro-phenyl)-acetamide; N-(2-Diethylamino-7-fluoro-4-oxo-4H-quinazolin-3-yl)-2-(3,5-difluoro-phenyl)-acetamide; N-(2-Diethylamino-5-fluoro-4-oxo-4H-quinazolin-3-yl)-2-(3,5-difluoro-phenyl)-acetamide; N-(2-Diethylamino-4-oxo-7-trifluoromethyl-4H-quinazolin-3-yl)-2-(3,5-difluoro-phenyl)-acetamide; N-(7-Chloro-2-diethylamino-4-oxo-4H-quinazolin-3-yl)-2-(3,5-difluoro-phenyl)-acetamide; N-(7-Chloro-4-oxo-2-pyrrolidin-1-yl-4H-quinazolin-3-yl)-2-(3,5-difluoro-phenyl)-acetamide; N-(6-Chloro-2-diethylamino-7-fluoro-4-oxo-4H-quinazolin-3yl)-2-(3,5-difluoro-phenyl)-acetamide; N-(2-Diethylamino-5,7-difluoro-4-oxo-4H-quinazolin-3-yl)-2-(3,5-difluoro-phenyl)-acetamide; 2-(3,5-Difluoro-phenyl)-N-(7-fluoro-4-oxo-2-pyrrolidin-1-yl-4H-quinazolin-3-yl)-acetamide; N-(6-Chloro-7-fluoro-4-oxo-2-pyrrolidin-1-yl-4H-quinazolin-3-yl)-2-(3,5-difluoro-phenyl)-acetamide; N-(5,7-Difluoro-4-oxo-2-pyrrolidin-1-yl-4H-quinazolin-3-yl)-2-(3,5-difluoro-phenyl)-acetamide; N-(5,7-Dichloro-2-diethylamino-4-oxo-4H-quinazolin-3-yl)-2-(3,5-difluoro-phenyl)-acetamide; N-(2-Diethylamino-6,7-difluoro-4-oxo-4H-quinazolin-3-yl)-2-(3,5-difluoro-phenyl)-acetamide; N-(6,7-Difluoro-4-oxo-2-pyrrolidin-1-yl-4H-quinazolin-3-yl)-2-(3,5-difluoro-phenyl)-acetamide; 2-(3-Fluoro-4-trifluoromethyl-phenyl)-N-(2-morpholin-4-yl-4-oxo-4H-quinazolin-3-yl)-acetamide; N-(2-Dimethylamino-4-oxo-4H-quinazolin-3-yl)-2-(3-fluoro-4-trifluoromethyl-phenyl)-acetamide; N-(6-Chloro-2-diethylamino-4-oxo-4H-quinazolin-3-yl)-2-(3,5-difluoro-phenyl)-acetamide; N-(2-Dimethylamino-6-fluoro-4-oxo-4H-quinazolin-3-yl)-2-(3-fluoro-4-trifluoromethyl-phenyl)-acetamide; N-(6-Fluoro-4-oxo-2-pyrrolidin-1-yl-4H-quinazolin-3-yl)-2-(3-fluoro-4-trifluoromethyl-phenyl)-acetamide; 2-(4-Chloro-3-fluoro-phenyl)-N-(6-fluoro-4-oxo-2-pyrrolidin-1-yl-4H-quinazolin-3-yl)-acetamide; 2-(3,5-Difluoro-phenyl)-N-(6-fluoro-4-oxo-2-pyrrolidin-1-yl-4H-quinazolin-3-yl)-acetamide; 2-(4-Chloro-3-fluoro-phenyl)-N-(2-dimethylamino-6-fluoro-4-oxo-4H-quinazolin-3-yl)-acetamide; 2-(3,5-Difluoro-phenyl)-N-(2-dimethylamino-6-fluoro-4-oxo-4H-quinazolin-3-yl)-acetamide; N-(5,7-Dichloro-4-oxo-2-pyrrolidin-1-yl-4H-quinazolin-3-yl)-2-(3,5-difluoro-phenyl)-acetamide; 2-(3,5-Difluoro-phenyl)-N-(2-dimethylamino-5-fluoro-4-oxo-4H-quinazolin-3-yl)-acetamide; 2-(3,5-Difluoro-phenyl)-N-(2-dimethylamino-7-fluoro-4-oxo-4H-quinazolin-3-yl)-acetamide; N-(2-Dimethylamino-5-fluoro-4-oxo-4H-quinazolin-3-yl)-2-(3-fluoro-5-trifluoromethyl-phenyl)-acetamide; N-(2-Dimethylamino-7-fluoro-4-oxo-4H-quinazolin-3-yl)-2-(3-fluoro-4-trifluoromethyl-phenyl)-acetamide; 2-(4-Chloro-phenyl)-N-(2-diethylamino-5,7-difluoro-4-oxo-4-quinazolin-3-yl)-acetamide; 2-(4-Chloro-phenyl)-N-(7-fluoro-4-oxo-2-pyrrolidin-1yl-4H-quinazolin-3-yl)-acetamide; 2-(3,5-Difluoro-phenyl)-N-[5-fluoro-2-(isopropyl-methyl-amino)-4-oxo-4H-quinazolin-3-yl]-acetamide; 2-(3,5-Difluoro-phenyl)-N-[2-(ethyl-methyl-amino)-5-fluoro-4-oxo-4H-quinazolin-3-yl]-acetamide; 2-(3,5-Difluoro-phenyl)-N-[7-fluoro-2-(isopropyl-methyl-amino)-4-oxo-4H-quinazolin-3-yl]-acetamide; 2-(3,5-Difluoro-phenyl)-N-[2-(ethyl-methyl-amino)-7-fluoro-4-oxo-4H-quinazolin-3-yl]-acetamide; 2-(4-Chloro-phenyl)-N-(7-fluoro-4-oxo-2-pyrrolidin-1-yl-4H-quinazolin-3-yl)-propionamide; 2-(4-Chloro-phenyl)-N-(4-oxo-2-pyrrolidin-1-yl-4H-quinazolin-3-yl)-propionamide; N-(2-Diethylamino-8-fluoro-4-oxo-4H-quinazolin-3-yl)-2-(3,5-difluoro-phenyl)-acetamide; 2-(3,5-Difluoro-phenyl)-N-(8-fluoro-4-oxo-2-pyrrolidin-1-yl-4H-quinazolin-3-yl)-acetamide; 2-Naphthalen-2-yl-N-(4-oxo-2-pyrrolidin-1-yl-4H-quinazolin-3-yl)-acetamide; N-(5,7-Difluoro-4-oxo-2-pyrrolidin-1-yl-4H-quinazolin-3-yl)-2-(3,4-difluoro-phenyl)-acetamide; 2-(3,4-Difluoro-phenyl)-N-[2-(ethyl-methyl-amino)-7-fluoro-4-oxo-4H-quinazolin-3-yl]-acetamide; 2-(4-Chloro-phenyl)-N-[2-(ethyl-methyl-amino)-7-fluoro-4-oxo-4H-quinazolin-3-yl]-acetamide; 2-Naphthalen-1-yl-N-(4-oxo-2-pyrrolidin-1-yl-4H-quinazolin-3-yl)-acetamide; 2-(4-Chloro-phenyl)-N-(5,7-difluoro-4-oxo-2-pyrrolidin-1-yl-4H-quinazolin-3-yl)-acetamide; 2-(3,5-Difluoro-phenyl)-N-[2-(ethyl-methyl-amino)-5,7-difluoro-4-oxo-4H-quinazolin-3-yl]-acetamide; N-[5,7-Difluoro-2-(isopropyl-methyl-amino)-4-oxo-4H-quinazolin-3-yl]-2-(3,4-difluoro-phenyl)-acetamide; N-[5,7-Difluoro-2-(isopropyl-methyl-amino)-4-oxo-4H-quinazolin-3-yl]-2-(3,5-difluoro-phenyl)-acetamide; N-[2-(Ethyl-methyl-amino)-7-fluoro-4-oxo-4H-quinazolin-3-yl]-2-(4-methylsulfanyl-phenyl)-acetamide; 2-(4-Isobutyl-phenyl)-N-(4-oxo-2-pyrrolidin-1-yl-4H-quinazolin-3-yl)-propionamide; N-[2-(Ethyl-methyl-amino)-7-fluoro-4-oxo-4H-quinazolin-3-yl]-2-(4-methoxy-phenyl)-acetamide; N-(7-Fluoro-4-oxo-2-pyrrolidin-1-yl-4H-quinazolin-3-yl)-2-(4-phenoxy-phenyl)-acetamide;

2-Biphenyl-4-yl-N-(7-fluoro-4-oxo-2-pyrrolidin-1-yl-4H-quinazolin-3-yl)-acetamide; 2-(4-Chloro-phenyl)-N-(4-oxo-2-pyrrolidin-1-yl-4H-quinazolin-3-yl)-acetamide; 2-(3,4-Difluoro-phenyl)-N-(4-oxo-2-pyrrolidin-1-yl-4H-quinazolin-3-yl)-acetamide; N-[2-(Ethyl-methyl-amino)-7-fluoro-4-oxo-4H-quinazolin-3-yl]-2-(4-isopropyl-phenyl)-acetamide; 2-(3,5-Difluoro-phenyl)-N-[2-(isopropyl-methyl-amino)-4-oxo-4-quinazolin-3-yl]-acetamide; 2-(3,4-Difluoro-phenyl)-N-[2-(isopropyl-methyl-amino)-4-oxo-4H-quinazolin-3-yl]-acetamide; 2-(3,4-Difluoro-phenyl)-N-(7-fluoro-4-oxo-2-pyrrolidin-1-yl-4H-quinazolin-3-yl)-acetamide; 2-(3,4-Difluoro-phenyl)-N-(8-fluoro-4-oxo-2-pyrrolidin-1-yl-4H-quinazolin-3-yl)-acetamide; 2-(3,4-Difluoro-phenyl)-N-[2-(isobutyl-methyl-amino)-4-oxo-4H-quinazolin-3-yl]-acetamide; 2-(3,5-Difluoro-phenyl)-N-[2-(isobutyl-methyl-amino)-4-oxo-4H-quinazolin-3-yl]-acetamide; 2-(3,4-Difluoro-phenyl)-N-[7-fluoro-2-(isopropyl-methyl-amino)-4-oxo-4H-quinazolin-3-yl]-acetamide; N-(7-Chloro-6-fluoro-4-oxo-2-pyrrolidin-1-yl-4H-quinazolin-3-yl)-2-(3,5-difluoro-phenyl)-acetamide; 2-(3,5-Difluoro-phenyl)-N-(2-diisopropylamino-4-oxo-4H-quinazolin-3-yl)-acetamide; N-[2-(Cyclopentyl-methyl-amino)-4-oxo-4H-quinazolin-3-yl]-2-(3,5-difluoro-phenyl)-acetamide; N-[2-(Cyclopentyl-methyl-amino)-4-oxo-4H-quinazolin-3-yl]-2-(3,4-difluoro-phenyl)-acetamide; 2-(3,4-Difluoro-phenyl)-N-(2-diisopropylamino-4-oxo-4H-quinazolin-3-yl)-acetamide; 2-(3,4-Difluoro-phenyl)-N-(4-oxo-2-piperidin-1-yl-4H-quinazolin-3-yl)-acetamide; 2-(3,5-Difluoro-phenyl)-N-(4-oxo-2-piperidin-1-yl-4H-quinazolin-3-yl)-acetamide; N-(8-Chloro-4-oxo-2-pyrrolidin-1-yl-4H-quinazolin-3-yl)-2-(3,5-difluoro-phenyl)-acetamide; N-(2-Diethylamino-6-hydroxy-4-oxo-4H-quinazolin-3-yl)-2-(3,5-difluoro-phenyl)-acetamide; N-(6,8-Dichloro-2-diethylamino-4-oxo-4H-quinazolin-3-yl)-2-(3,5-difluoro-phenyl)-acetamide; N-(6-Amino-2-diethylamino-4-oxo-4H-quinazolin-3-yl)-2-(3,5-difluoro-phenyl)-acetamide; N-(7-Amino-2-diethylamino-4-oxo-4H-quinazolin-3-yl)-2-(3,5-difluoro-phenyl)-acetamide; N-(7-Amino-4-oxo-2-pyrrolidin-1-yl-4H-quinazolin-3-yl)-2-(3,5-difluoro-phenyl)-acetamide; or a stereoisomer or a mixture of its stereoisomers, a pharmaceutically-acceptable addition salt thereof.

In further embodiments, 2-(3,5-difluorophenyl)-N-(2-dimethylamino-4-oxo-7-trifluoromethyl-4H-quinazolin-3-yl)-propionamide; or a stereoisomer or a mixture of its stereoisomers, or a pharmaceutically-acceptable addition salt thereof.

### Formula 40

In an embodiment, the Kv7 channel activator may be selected from one of the following compounds according to Formula 40. Such compounds are described in International Publication No. WO2010060955A1, published Jun. 3, 2010 and corresponding to International Application No. PCT/EP2009/065890 filed Nov. 26, 2009; US Publication No. US20110312962A1, published Dec. 22, 2011 and corresponding to U.S. application Ser. No. 13/131,218 filed Nov. 26, 2009; which are incorporated by reference in their entirety herein. In the case of any conflict of terminology in the context of Formula 40, these references incorporated by reference herein control.

In an embodiment, the Kv7 channel activator is a compound according to Formula 40:

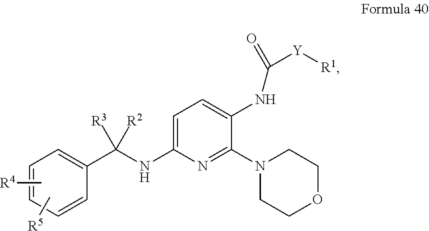

a stereoisomer or a mixture of its stereoisomers, or a pharmaceutically acceptable addition salt thereof, or an N-oxide thereof, wherein Y represents —(CH₂)ₙ—, —(CH₂)ₙ—O— or —(CH₂)ₙ—S— wherein n is 0 or 1; R¹ represents C₁₋₆-alkyl, benzo[1,3]dioxolyl, phenyl or pyridyl, which phenyl and pyridyl are optionally substituted one or more times with substituents selected from the group consisting of C₁₋₆-alkyl, halogen, trifluoromethyl, hydroxy, C₁₋₆-alkoxy and trifluoromethoxy; R² and R³, independently of each other, represent hydrogen or C₁₋₆-alkyl; and R⁴ and R⁵, independently of each other, represent hydrogen, C₁₋₆-alkyl, halogen, trifluoromethyl, hydroxy, C₁₋₆-alkoxy or trifluoromethoxy.

In further embodiments, Y represents —(CH₂)ₙ—, wherein n is 0 or 1; R¹ represents C₁₋₆-alkyl, benzo[1,3]dioxolyl or phenyl, which phenyl is optionally substituted one or more times with substituents selected from the group consisting of C₁₋₆-alkyl, halogen, trifluoromethyl, hydroxy, C₁₋₆-alkoxy and trifluoromethoxy; R² and R³, independently of each other, represent hydrogen or C₁₋₆-alkyl; and R⁴ and R⁵, independently of each other, represent hydrogen, C₁₋₆-alkyl, halogen, trifluoromethyl, hydroxy, C₁₋₆-alkoxy or trifluoromethoxy. In further embodiments, n is 1.

In further embodiments, R¹ represents phenyl optionally substituted one or more times with substituents selected from the group consisting of C₁₋₆-alkyl, halogen, trifluoromethyl and C₁₋₆-alkoxy.

In further embodiments, R² and R³ represent hydrogen.

In further embodiments, R² and R³ represent C₁₋₆-alkyl.

In further embodiments, R⁴ and R⁵, independently of each other, represent hydrogen or halogen.

In further embodiments, the Kv7 Channel activator is selected from the group consisting of: 2-(3,5-Difluoro-phenyl)-N-[6-(4-fluoro-benzylamino)-2-morpholin-4-yl-pyridin-3-yl]-acetamide; 2-(3,5-Difluoro-phenyl)-N-{6-[1-(4-fluoro-phenyl)-1-methyl-ethylamino]-2-morpholin-4-yl-pyridin-3-yl}-acetamide; N-[6-(4-Fluoro-benzylamino)-2-morpholin-4-yl-pyridin-3-yl]-2-methyl-benzamide; 2-Benzo[1,3]dioxol-5-yl-N-[6-(4-fluoro-benzylamino)-2-morpholin-4-yl-pyridin-3-yl]-acetamide; N-[6-(4-Fluoro-benzylamino)-2-morpholin-4-yl-pyridin-3-yl]-2-(4-methoxy-phenyl)-acetamide; N-[6-(4-Fluoro-benzylamino)-2-morpholin-4-yl-pyridin-3-yl]-2-(3-methoxy-phenyl)-acetamide; 2-(2,4-Dichloro-phenyl)-N-[6-(4-fluoro-benzylamino)-2-morpholin-4-yl-pyridin-3-yl]-acetamide; N-[6-(4-Fluoro-benzylamino)-2-morpholin-4-yl-pyridin-3-yl]-2-(3-trifluoromethyl-phenyl)-acetamide; N-[6-(4-Fluoro-benzylamino)-2-morpholin-4-yl-pyridin-3-yl]-2-(2-fluoro-phenyl)-acetamide; N-[6-(4-Fluoro-benzylamino)-2-morpholin-4-yl-pyridin-3-yl]-2-(3-fluoro-phenyl)-acetamide; or 2-(4-Chloro-phenyl)-N-[6-(4-fluoro-benzylamino)-2-morpholin-4-yl-pyridin-3-yl]-acetamide, N-[6-[(4-Fluorophenyl)-methylamino]-2-morpholino-3-pyridyl]-2-(4-fluorophenyl)-sulfanyl-acetamide; 2-(3-fluorophenoxy)-N-[6-[(4-fluorophenyl)-methyl amino]-2-morpholino-3-pyridyl]acetamide; 2-(6-chloro-3-pyridyl)-N-[6-[(4-fluorophenyl)-methylamino]-2-morpholino-3-pyridyl]acetamide; 2-fluoro-N-[6-[(4-fluorophenyl)-methylamino]-2-morpholino-3-pyridyl]pyridine-3-carboxamide; a stereoisomer or a mixture of its stereoisomers, or a pharmaceutically acceptable addition salt thereof, or an N-oxide thereof.

### Formula 41

In an embodiment, the Kv7 channel activator may be selected from one of the following compounds according to Formula 41. Such compounds are described in US Publication No. US20110269783A1, published Nov. 3, 2011, and corresponding to U.S. application Ser. No. 13/128,015 filed Nov. 6, 2009; International Publication No. WO2010051819A1, published May 14, 2010, and corresponding to International Application No. PCT/DK2009/050293 filed Nov. 6, 2009; which are incorporated by reference in their entirety herein. In the case of any conflict of terminology in the context of Formula 41, these references incorporated by reference herein control.

In an embodiment, the Kv7 channel activator is a compound according to Formula 41:

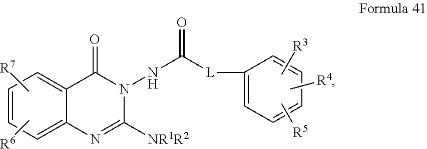

A stereoisomer or a mixture of its stereoisomers, or a pharmaceutically acceptable addition salt thereof, or an N-oxide thereof, wherein L represents a linker selected from —(CR′R″)₂—, —CR′R″—S—, —CR′R″—O— or

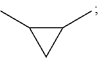

wherein R′ and R″, independently of each other, represent hydrogen, C₁₋₆-alkyl or halogen; R¹ and R², independently of each other, represent C₁₋₆-alkyl, hydroxy-C₁₋₆-alkyl-, C₁₋₆-alkoxy-C₁₋₆-alkyl-, phenyl, phenyl-C₁₋₆-alkyl-, which phenyl is optionally substituted with one or two times with a substituent selected from the group consisting of C₁₋₆-alkoxy, halogen and cyano; or R¹ and R², together with the nitrogen to which they are attached, form a heterocyclic ring selected from pyrrolidinyl, 2,5-dihydro-1H-pyrrol-1-yl, thiazolidinyl, piperidinyl, piperazinyl and morpholinyl, which pyrrolidinyl, piperidinyl, piperazinyl and morpholinyl is optionally substituted one or more times with a substituent selected from the group consisting of halogen, hydroxy, amino, C₁₋₆-alkyl, trifluoromethyl, C₁₋₆-alkoxy, hydroxy-C₁₋₆-alkyl- and C₁₋₆-alkoxy-C₁₋₆-alkyl-; R³, R⁴ and R⁵, independently of each other, represent hydrogen, C₁₋₆-alkyl, halogen, trihalomethyl, hydroxy, C₁₋₆-alkoxy, trifluoromethoxy, amino, cyano or nitro; and R⁶ and R⁷, independently of each other, represent hydrogen, C₁₋₆-alkyl, halogen, trihalomethyl, hydroxy, C₁₋₆-alkoxy, trifluoromethoxy, amino, nitro, cyano or phenyl.

In further embodiments, R¹ and R², independently of each other, represent C₁₋₆-alkyl, alkoxy-C₁₋₆-alkyl- or phenyl-C₁₋₆-alkyl-.

In further embodiments, R¹ and R², together with the nitrogen to which they are attached, form a pyrrolidinyl ring.

In further embodiments, R³, R⁴ and R⁵, independently of each other, represent hydrogen, C₁₋₆-alkyl or halogen.

In further embodiments, R⁶ and R⁷, independently of each other, represent hydrogen or halogen.

In further embodiments, the Kv7 Channel activator is selected from the group consisting of: N-(7-Fluoro-4-oxo-2-pyrrolidin-1-yl-4H-quinazolin-3-yl)-3-(3-fluoro-phenyl)-propionamide; 3-(3-Fluoro-phenyl)-N-{2-[(2-methoxy-ethyl)-methyl-amino]-4-oxo-4H-quinazolin-3-yl}-propionamide; N-[2-(Benzyl-methyl-amino)-4-oxo-4H-quinazolin-3-yl]-3-(3-fluoro-phenyl)-propionamide; N-[2-(Benzyl-methyl-amino)-4-oxo-4H-quinazolin-3-yl]-3-(3,5-difluoro-phenyl)-propionamide; 3-(3-Fluoro-phenyl)-N-(4-oxo-2-pyrrolidin-1-yl-4H-quinazolin-3-yl)-propionamide; N-(4-Oxo-2-pyrrolidin-1-yl-4H-quinazolin-3-yl)-3-phenyl-propionamide; N-(2-Dimethylamino-7-fluoro-4-oxo-4H-quinazolin-3-yl)-3-(3-fluoro-phenyl)-propionamide; N-(4-Oxo-2-pyrrolidin-1-yl-4H-quinazolin-3-yl)-2-phenylsulfanyl-acetamide; N-(4-Oxo-2-pyrrolidin-1-yl-4H-quinazolin-3-yl)-2-phenoxy-acetamide; N-(5-Fluoro-4-oxo-2-pyrrolidin-1-yl-4H-quinazolin-3-yl)-3-(3-fluoro-phenyl)-propionamide; 2-(4-Fluoro-phenylsulfanyl)-N-(4-oxo-2-pyrrolidin-1-yl-4H-quinazolin-3-yl)-acetamide; N-(5,7-Difluoro-4-oxo-2-pynolidin-1-yl-4H-quinazolin-3-yl)-3-(3-fluoro-phenyl)-propionamide; 2-(4-tert-Butyl-phenylsulfanyl)-N-(4-oxo-2-pyrrolidin-1-yl-4H-quinazolin-3-yl)-acetamide; N-[5,7-Difluoro-2-(isopropyl-methyl-amino)-4-oxo-4H-quinazolin-3-yl]-3-(3-fluoro-phenyl)-propionamide; N-(5,7-Difluoro-4-oxo-2-pyrrolidin-1-yl-4H-quinazolin-3-yl)-3-(3,5-difluoro-phenyl)-propionamide; (S)—N-(5,7-Difluoro-4-oxo-2-pyrrolidin-1-yl-4H-quinazolin-3-yl)-3-phenyl-butyramide; 3-(4-Chloro-phenyl)-N-(4-oxo-2-pyrrolidin-1-yl-4H-quinazolin-3-yl)-butyramide; 3-(3-Fluoro-phenyl)-N-(4-oxo-2-pyrrolidin-1-yl-4H-quinazolin-3-yl)-butyramide; cis-2-(4-Chloro-phenyl)-cyclopropanecarboxylic acid (2-dimethylamino-7-fluoro-4-oxo-4H-quinazolin-3-yl)-amide; cis-2-(4-Chloro-phenyl)-cyclopropanecarboxylic acid [5,7-difluoro-2-(isopropyl-methyl-amino)-4-oxo-4H-quinazolin-3-yl]-amide; cis-2-(4-Chloro-phenyl)-cyclopropanecarboxylic acid (5,7-difluoro-4-oxo-2-pyrrolidin-1-yl-4H-quinazolin-3-yl)-amide; cis-2-(4-Chloro-phenyl)-cyclopropanecarboxylic acid [2-(ethyl-methyl-amino)-7-fluoro-4-oxo-4H-quinazolin-3-yl]-amide; cis-2-(4-Chloro-phenyl)-cyclopropanecarboxylic acid [7-fluoro-2-(isopropyl-methyl-amino)-4-oxo-4H-quinazolin-3-yl]-amide; cis-2-(4-Chloro-phenyl)-cyclopropanecarboxylic acid [2-(ethyl-methyl-amino)-5,7-difluoro-4-oxo-4H-quinazolin-3-yl]-amide; cis-2-(4-Chloro-phenyl)-cyclopropanecarboxylic acid (8-fluoro-4-oxo-2-pyrrolidin-1-yl-4H-quinazolin-3-yl)-amide; or a stereoisomer or a mixture of its stereoisomers, a pharmaceutically acceptable addition salt thereof, or an N-oxide thereof.

In further embodiments, the compound is of formula:

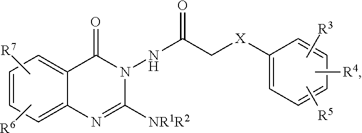

wherein: X represents —CR′R″—, —S—, or —O—, wherein R′ and R″, independently of each other, represent hydrogen, C₁₋₆-alkyl or halogen, and R′, R², R³, R⁴, R⁵, R⁶ and R⁷ are as defined above.

In further embodiments, the compound is of formula:

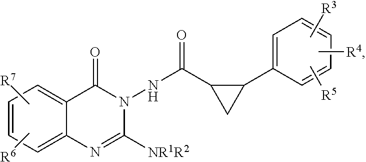

wherein: R¹, R², R³, R⁴, R⁵, R⁶ and R⁷ are as defined above.

### Formula 42

In another embodiment, the Kv7 channel activator may be selected from one of the following compounds. Such compounds are described in International Publication No. WO2011026890A1, published Mar. 10, 2011, and corresponding to International Application No. PCT/EP2010/062859 filed Sep. 2, 2010; US Publication No.

US20120238547A1, published Sep. 20, 2012, and corresponding to U.S. application Ser. No. 13/394,345 filed Sep. 2, 2010; which are incorporated by reference in their entirety herein. In the case of any conflict of terminology in the context of Formula 42, these references incorporated by reference herein control.

In an embodiment, the Kv7 channel activator is a compound according to Formula 42:

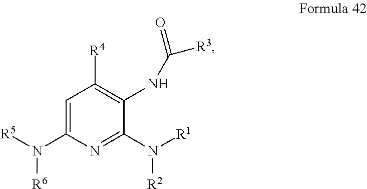

a stereoisomer or a mixture of its stereoisomers, or a pharmaceutically acceptable addition salt thereof, or an N-oxide thereof, wherein R¹ and R², together with the nitrogen to which they are attached, form a heterocyclic ring selected from pyrrolidinyl, 2,5-dihydro-1H-pyrrol-1-yl, azetidinyl, thiazolidinyl, piperidinyl, piperazinyl and morpholinyl, which pyrrolidinyl, azetidinyl, piperidinyl, piperazinyl and morpholinyl is optionally substituted one or more times with a substituent selected from the group consisting of halo and trifluoromethyl; R³ represents a heterocyclic ring selected from furanyl and pyrrolyl which furanyl and pyrrolyl is optionally substituted one or more times with substituents selected from C₁₋₆-alkyl, C₁₋₆-alkoxy, halo and trifluoromethyl; R⁴ represents C₁₋₆-alkyl; and R⁵ and R⁶, together with the nitrogen to which they are attached, form a heterocyclic ring selected from morpholinyl and 1,4-oxazepanyl.

In further embodiments, R¹ and R² together with the nitrogen to which they are attached is pyrrolidinyl which is optionally substituted one or more times with halo.

In further embodiments, R³ represents furanyl which is optionally substituted with C₁₋₆-alkyl.

In further embodiments, R⁴ represents C₁₋₆-alkyl.

In further embodiments, R⁵ and R⁶, together with the nitrogen to which they are attached, represents morpholinyl.

In further embodiments, the Kv7 Channel activator is selected from the group consisting of: 3-Methyl-furan-2-carboxylic acid [2-((R)-3-fluoro-pyrrolidin-1-yl)-4-methyl-6-[1,4]oxazepan-4-yl-pyridin-3-yl]-amide; 3-Methyl-furan-2-carboxylic acid [2-((R)-3-fluoro-pyrrolidin-1-yl)-4-methyl-6-morpholin-4-yl-pyridin-3-yl]-amide; or a stereoisomer or a mixture of its stereoisomers, or a pharmaceutically acceptable addition salt thereof, or an N-oxide thereof.

### Formula 43

In an embodiment, the Kv7 channel activator may be selected from one of the following compounds according to Formula 43. Such compounds are described in International Publication No. WO2010026104A1, published Mar. 11, 2010, and corresponding to International Application No. PCT/EP2009/061125 filed Aug. 28, 2009; which is incorporated by reference in its entirety herein. In the case of any conflict of terminology in the context of Formula 43, the reference incorporated by reference herein controls.

In an embodiment, the Kv7 channel activator is a compound according to Formula 43:

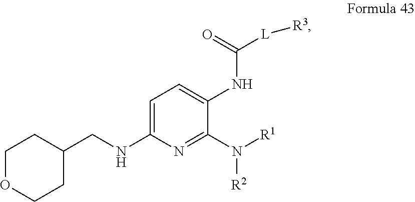

a stereoisomer or a mixture of its stereoisomers, or a pharmaceutically-acceptable addition salt thereof, or an N-oxide thereof, wherein R¹ and R², independently of each other, represent Ci₋₆-alkyl, hydroxy-d-e-alkyl or Ci₋₆-alkoxy-Ci₋₆-alkyl; or R¹ and R², together with the nitrogen to which they are attached, form a heterocyclic ring selected from pyrrolidinyl, 2,5-dihydro-1H-pyrrol-1-yl, azetidinyl, thiazolidinyl, piperidinyl, piperazinyl and morpholinyl, which pyrrolidinyl, azetidinyl, piperidinyl, piperazinyl and morpholinyl is optionally substituted one or more times with a substituent selected from the group consisting of halo, hydroxy, amino, Ci₋₆-alkyl, trifluoromethyl, Ci₋₆-alkoxy, hydroxy-Ci₋₆-alkyl and Ci₋₆-alkoxy-Ci₋₆-alkyl; R³ represents Ci₋₆-alkyl or phenyl, which phenyl is optionally substituted one or more times with substituents selected from Ci₋₆-alkyl, Ci₋₆-alkoxy, halo and trifluoromethyl; and L represents a linker selected from —CR¹R″—, —CH₂—CR¹R″— and —CR¹R¹\hat{ }CH₂—, wherein R¹ and R″, independently of each other, represent hydrogen, Ci₋₆-alkyl or halo.

In further embodiments, R¹ and R², independently of each other, represent Ci₋₆-alkyl.

In further embodiments, R¹ and R², together with the nitrogen to which they are attached, form a heterocyclic ring selected from pyrrolidinyl, azetidinyl, piperidinyl and morpholinyl, which pyrrolidinyl, azetidinyl, piperidinyl and morpholinyl is optionally substituted one or more times with a substituent selected from the group consisting of halo, d-e-alkyl and trifluoromethyl.

In further embodiments, R¹ and R², together with the nitrogen to which they are attached, form a heterocyclic ring which is pyrrolidinyl optionally substituted one or more times with a substituent selected from the group consisting of halo, d-e-alkyl and trifluoromethyl.

In further embodiments, R¹ and R², together with the nitrogen to which they are attached, form a heterocyclic ring which is piperidinyl optionally substituted one or more times with a substituent selected from the group consisting of halo, d-e-alkyl and trifluoromethyl.

In further embodiments, R³ represents the/t-butyl.

In further embodiments, R³ represents phenyl, which is optionally substituted one or more times with substituents selected from Ci₋₆-alkyl, Ci₋₆-alkoxy, halo and trifluoromethyl. In further embodiments, L represents —CH₂-.

In further embodiments, the Kv7 Channel activator is selected from the group consisting of: 2-(3,5-Difluorophenyl)-N-{2-pyrrolidin-1-yl-6-[(tetrahydro-pyran-4-ylmethyl)-amino]-pyridin-3-yll-acetamide; 3,3-Dimethyl-N-{2-pyrrolidin-1-yl-6-[(tetrahydro-pyran-4-ylmethyl)-amino]-pyridin-3-yllbutyramide; 2-(3,5-Difluoro-phenyl)-N-{2-morpholin-4-yl-6-[(tetrahydro-pyran-4-ylmethyl)-amino]-pyridin-3-yll-acetamide; 2-(3,5-Difluoro-phenyl)-N-{2-dimethylamino-6-[(tetrahydro-pyran-4-ylmethyl)-amino]-pyridin-3-yll-acetamide; N-{2-Dimethylamino-6-[(tetrahydro-pyran-4-ylmethyl)-amino]-pyridin-3-yll-3,3-dimethyl-butyramide; 2-(3,5-Difluoro-phenyl)-N-{2-((S)-3-fluoro-pyrrolidin-1-yl)-6-[(tetrahydro-pyran-4-ylmethyl)-amino]-pyridin-3-yll-acetamide; N-{2-((S)-3-Fluoro-pyrrolidin-1-yl)-6-[(tetrahydro-pyran-4-ylmethyl)-amino]-pyridin-3-yll-3,3-dimethyl-butyramide; 2-(3,5-Difluoro-phenyl)-N-{2-(3,3-dimethyl-pyrrolidin-1-yl)-6-[(tetrahydro-pyran-4-ylmethyl)-amino]-pyridin-3-yll-acetamide; N-{2-(3,3-Dimethyl-pyrrolidin-1-yl)-6-[(tetrahydro-pyran-4-ylmethyl)-amino]-pyridin-3-yll-3,3-dimethyl-butyramide; N-{2-((R)-3-Fluoro-pyrrolidin-1-yl)-6-[(tetrahydro-pyran-4-ylmethyl)-amino]-pyridin-3-yll-3,3-dimethyl-butyramide; 2-(3,5-Difluoro-phenyl)-N-{2-((R)-3-fluoro-pyrrolidin-1-yl)-6-[(tetrahydro-pyran-4-ylmethyl)-amino]-pyridin-3-yll-acetamide; 2-(3,5-Difluoro-phenyl)-N-[6-[(tetrahydro-pyran-4-ylmethyl)-amino]-2-((S)-2-trifluoromethyl-pyrrolidin-1-yl)-pyridin-3-yl]-acetamide; 3,3-Dimethyl-N-[6-[(tetrahydro-pyran-4-ylmethyl)-amino]-2-((S)-2-trifluoromethylpyrrolidin-1-yl)-pyridin-3-yl]-butyramide; or a pharmaceutically-acceptable addition salt thereof.

### Formula 44

In an embodiment, the Kv7 channel activator may be selected from one of the following compounds according to Formula 44. Such compounds are described in International Publication No. WO2010105960A1, published Sep. 23, 2010, and corresponding to International Application No. PCT/EP2010/053072 filed Mar. 11, 2010; US Publication No. US20110003865A1, published Jan. 6, 2011, and corresponding to U.S. application Ser. No. 12/747,394 filed Dec. 10, 2008; which are incorporated by reference in their entirety herein. In the case of any conflict of terminology in the context of Formula 44, these references incorporated by reference herein control.

In an embodiment, the Kv7 channel activator is a compound according to Formula 44:

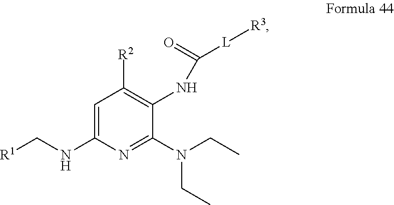

a stereoisomer or a mixture of its stereoisomers, or a pharmaceutically-acceptable addition salt thereof, or an N-oxide thereof, wherein R¹ represents alkyl or phenyl, which phenyl is optionally substituted one or more times with substituents selected from alkyl, halo and trifluoromethyl; R² represents hydrogen; R³ represents alkyl or phenyl, which phenyl is optionally substituted one or more times with substituents selected from alkyl, halo, alkoxy and trifluoromethyl; and L represents a linker selected from —CR′R″—, —CH₂—CR′R″—, —CR′R″—CH₂— and cycloalkyl, wherein R′ and R″, independently of each other, represent hydrogen, alkyl or halo.

In further embodiments, R¹ represents alkyl.

In further embodiments, R¹ represents phenyl optionally substituted one or more times with substituents selected from alkyl and halo.

In further embodiments, R³ represents phenyl, optionally substituted one or more times with substituents selected from alkyl, halo, alkoxy and trifluoromethyl.

In further embodiments, R³ represents alkyl.

In further embodiments, L represents a linker selected from —CR′R″—, —CH₂—CR′R″—, —CR′R″—CH₂— and cycloalkyl, wherein R′ and R″, independently of each other, represent hydrogen or alkyl.

In further embodiments, the Kv7 Channel activator is selected from the group consisting of: N-[2-Diethylamino-6-(4-fluoro-benzylamino)-pyridin-3-yl]-2-(3,5-difluoro-phenyl)-acetamide; N-[2-Diethylamino-6-(4-fluoro-benzylamino)-pyridin-3-yl]-2-(3,4-difluoro-phenyl)-acetamide; N-[2-Diethylamino-6-(4-fluoro-benzylamino)-pyridin-3-yl]-2-(3-fluoro-4-trifluoromethyl-phenyl)-acetamide; (S)—N-[2-Diethylamino-6-(4-fluoro-benzylamino)-pyridin-3-yl]-2-phenyl-propionamide; N-[2-Diethylamino-6-(4-fluoro-benzylamino)-pyridin-3-yl]-3-(3-fluoro-phenyl)-propionamide; (R)—N-[2-Diethylamino-6-(4-fluoro-benzylamino)-pyridin-3-yl]-3-phenyl-butyramide; trans-2-Phenyl-cyclopropanecarboxylic acid [2-diethylamino-6-(4-fluoro-benzylamino)-pyridin-3-yl]-amide; N-[2-Diethylamino-6-(4-fluoro-benzylamino)-pyridin-3-yl]-2-(3-fluoro-phenyl)-acetamide; N-[2-Diethylamino-6-(4-fluoro-benzylamino)-pyridin-3-yl]-3,3-dimethyl-butyramide; N-[2-Diethylamino-6-(2,4,6-trimethyl-benzylamino)-pyridin-3-yl]-2-(3,5-difluoro-phenyl)-acetamide; N-(2-Diethylamino-6-isobutylamino-pyridin-3-yl)-2-(3,5-difluoro-phenyl)-acetamide; N-[2-Diethylamino dimethyl-benzylamino)-pyridin-3-yl]-2-(3,5-difluoro-phenyl)-acetamide; N-[2-Diethylamino-6-(2,2-dimethyl-propylamino)-pyridin-3-yl]-2-(3,5-difluoro-phenyl)-acetamide; or a stereoisomer or a mixture of its stereoisomers, or a pharmaceutically-acceptable addition salt thereof, or an N-oxide thereof.

### Formula 45

In an embodiment, the Kv7 channel activator may be selected from one of the following compounds according to Formula 45. Such compounds are described in US Publication No. US20110039896A1, published Feb. 17, 2011, and corresponding to U.S. application Ser. No. 12/747,346 filed Dec. 10, 2008; International Publication No. WO2009074593 A1, published Jun. 18, 2009, and corresponding to International Application No. PCT/EP2008/067164 filed Dec. 10, 2008; which are incorporated by reference in their entirety herein. In the case of any conflict of terminology in the context of Formula 45, these references incorporated by reference herein control.

In an embodiment, the Kv7 channel activator is a compound according to Formula 45:

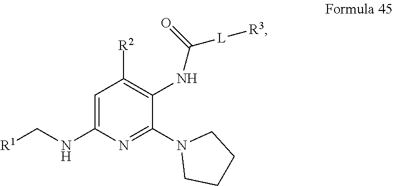

a stereoisomer or a mixture of its stereoisomers, or a pharmaceutically-acceptable addition salt thereof, or an N-oxide thereof, wherein R¹ represents alkyl, phenyl, benzo[1,3]dioxolyl or benzo[1,4]dioxinyl, which phenyl is optionally substituted one or more times with substituents selected from alkyl, halo, trifluoromethyl, alkoxy, cyano and difluoromethoxy; R² represents hydrogen; R³ represents alkyl or phenyl, which phenyl is optionally substituted one or more times with substituents selected from alkyl, halo, alkoxy and trifluoromethyl; and L represents a linker selected from —CR′R″—, —CH₂—CR′R″—, —CR′R″—CH₂— and cycloalkyl, wherein R′ and R″, independently of each other, represent hydrogen, alkyl or halo.

In further embodiments, R¹ represents alkyl or phenyl, which phenyl is optionally substituted one or more times with substituents selected from alkyl, halo and trifluoromethyl; R² represents hydrogen; R³ represents phenyl, which phenyl is optionally substituted one or more times with substituents selected from alkyl, halo and trifluoromethyl; and L represents a linker selected from —CR′R″—, —CH₂—CR′R″—, —CR′R″—CH₂— and cycloalkyl, wherein R′ and R″, independently of each other, represent hydrogen, alkyl or halo. In further embodiments, R¹ represents alkyl.

In further embodiments, R¹ represents phenyl, benzo[1,3]dioxolyl or
benzo[1,4]dioxinyl, which phenyl is optionally substituted one or more times with substituents selected from alkyl, halo, trifluoromethyl, alkoxy, cyano and difluoromethoxy.

In further embodiments, R³ represents alkyl.

In further embodiments, R³ represents phenyl, optionally substituted one or more
times with substituents selected from alkyl, halo, alkoxy and trifluoromethyl.

In further embodiments, L represents a linker selected from —CR′R″—, —CH₂—CR′R″—, —CR′R″—CH₂— and cycloalkyl, wherein R′ and R″, independently of each other, represent hydrogen or alkyl.

In further embodiments, the Kv7 Channel activator is selected from the group consisting of: N-[6-(4-Fluoro-benzylamino)-2-pyrrolidin-1-yl-pyridin-3-yl]-2-(3-fluoro-4-trifluoromethyl-phenyl)-acetamide; 2-(3,5-Difluoro-phenyl)-N-[6-(4-fluoro-benzylamino)-2-pyrrolidin-1-yl-pyridin-3-yl]-acetamide; 2-(3,4-Difluoro-phenyl)-N-[6-(4-fluoro-benzylamino)-2-pyrrolidin-1-yl-pyridin-3-yl]-acetamide; (S)—N-[6-(4-Fluoro-benzylamino)-2-pyrrolidin-1-yl-pyridin-3-yl]-2-phenyl-propionamide; N-[6-(4-Fluoro-benzylamino)-2-pyrrolidin-1-yl-pyridin-3-yl]-3-(3-fluoro-phenyl)-propionamide; (R)—N-[6-(4-Fluoro-benzylamino)-2-pyrrolidin-1-yl-pyridin-3-yl]-3-phenyl-butyramide; (R)-2-Phenyl-cyclopropanecarboxylic acid [6-(4-fluoro-benzylamino)-2-pyrrolidin-1-yl-pyridin-3-yl]-amide; N-[6-(4-Fluoro-benzylamino)-2-pyrrolidin-1-yl-pyridin-3-yl]-2-(3-fluoro-phenyl)-acetamide; N-[6-(4-Fluoro-benzylamino)-2-pyrrolidin-1-yl-pyridin-3-yl]-3,3-dimethyl-butyramide; 2-(3,5-Difluoro-phenyl)-N-(6-isobutylamino-2-pyrrolidin-1-yl-pyridin-3-yl)-acetamide; 2-(3,5-Difluoro-phenyl)-N-[6-(5-fluoro-2-methyl-benzylamino)-2-pyrrolidin-1-yl-pyridin-3-yl]-acetamide; 2-(3,5-Difluoro-phenyl)-N-[6-(2,6-dimethyl-benzylamino)-2-pyrrolidin-1-yl-pyridin-3-yl]-acetamide; 2-(3,5-Difluoro-phenyl)-N-[2-pyrrolidin-1-yl-6-(4-trifluoromethyl-benzylamino)-pyridin-3-yl]-acetamide; N-{6-[(Benzo[1,3]dioxol-5-ylmethyl)-amino]-2-pyrrolidin-1-yl-pyridin-3-yl}-3,3-dimethyl-butyramide; N-[6-(3,4-Difluoro-benzylamino)-2-pyrrolidin-1-yl-pyridin-3-yl]-2-(3,5-difluoro-phenyl)-acetamide; N-{6-[(Benzo[1,3]dioxol-5-ylmethyl)-amino]-2-pyrrolidin-1-yl-pyridin-3-yl}-2-(3,5-difluoro-phenyl)-acetamide; N-[6-(4-Cyano-benzylamino)-2-pyrrolidin-1-yl-pyridin-3-yl]-2-(3,5-difluoro-phenyl)-acetamide; N-[6-(3,4-Difluoro-benzylamino)-2-pyrrolidin-1-yl-pyridin-3-yl]-3,3-dimethyl-butyramide; 2-(3,5-Difluoro-phenyl)-N-[6-(4-methoxy-benzylamino)-2-pyrrolidin-1-yl-pyridin-3-yl]-acetamide; N-[6-(4-Difluoromethoxy-benzylamino)-2-pyrrolidin-1-yl-pyridin-3-yl]-2-(3,5-difluoro-phenyl)-acetamide; N-[6-(4-Difluoromethoxy-benzylamino)-2-pyrrolidin-1-yl-pyridin-3-yl]-3,3-dimethyl-butyramide; 2-(3,5-Difluoro-phenyl)-N-{6-[(2,3-dihydro-benzo[1,4]dioxin-6-ylmethyl)-amino]-2-pyrrolidin-1-yl-pyridin-3-yl}-acetamide; N-{6-[(2,3-Dihydro-benzo[1,4]dioxin-6-ylmethyl)-amino]-2-pyrrolidin-1-yl-pyridin-3-yl}-3,3-dimethyl-butyramide; or a stereoisomer or a mixture of its stereoisomers, or a pharmaceutically-acceptable addition salt thereof, or an N-oxide thereof.

### Formula 46

In an embodiment, the Kv7 channel activator may be selected from one of the following compounds according to Formula 46. Such compounds are described in International Publication No. WO2009074591A1, published Jun. 18, 2009, and corresponding to International Application No. PCT/EP2008/067161 filed Dec. 10, 2008; US Publication No. US20110003866A1, published Dec. 10, 2008, and corresponding to U.S. application Ser. No. 12/747,414 filed Dec. 10, 2008; which are incorporated by reference in their entirety herein. In the case of any conflict of terminology in the context of Formula 46, these references incorporated by reference herein control.

In an embodiment, the Kv7 channel activator is a compound according to Formula 46:

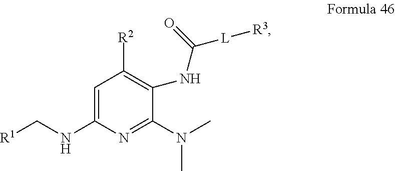

a stereoisomer or a mixture of its stereoisomers, or a pharmaceutically-acceptable addition salt thereof, or an N-oxide thereof, wherein R¹ represents alkyl or phenyl, which phenyl is optionally substituted one or more times with substituents selected from alkyl, halo and trifluoromethyl; R² represents hydrogen; R³ represents alkyl or phenyl, which phenyl is optionally substituted one or more times with substituents selected from alkyl, halo, alkoxy and trifluoromethyl; and L represents a linker selected from —CR′R″—, —CR′R″—CH₂— and cycloalkyl, wherein R′ and R″, independently of each other, represent hydrogen, alkyl or halo.

In further embodiments, R¹ represents alkyl.

In further embodiments, R¹ represents phenyl, which phenyl is optionally substituted one or more times with substituents selected from alkyl, halo and trifluoromethyl.

In further embodiments, R³ represents alkyl.

In further embodiments, R³ represents phenyl, which phenyl is optionally substituted one or more times with substituents selected from alkyl, halo, alkoxy and trifluoromethyl.

In further embodiments, L represents a linker selected from —CR′R″—, —CR′R″—CH₂— and cycloalkyl, wherein R′ and R″, independently of each other, represent hydrogen, alkyl or halo.

In further embodiments, the Kv7 Channel activator is selected from the group consisting of: N-[2-Dimethylamino-6-(4-fluoro-benzylamino)-pyridin-3-yl]-2-(3,4-difluoro-phenyl)-acetamide; N-[2-Dimethylamino-6-(4-fluoro-benzylamino)-pyridin-3-yl]-2-(3,5-difluoro-phenyl)-acetamide; N-[2-Dimethylamino-6-(4-fluoro-benzylamino)-pyridin-3-yl]-2-(3-fluoro-4-trifluoromethyl-phenyl)-acetamide; (S)—N-[2-Dimethylamino-6-(4-fluoro-benzylamino)-pyridin-3-yl]-2-phenyl-propionamide; N-[2-Dimethylamino-6-(4-fluoro-benzylamino)-pyridin-3-yl]-3-(3-fluoro-phenyl)-propionamide; trans-2-Phenyl-cyclopropanecarboxylic acid [2-dimethylamino-6-(4-fluoro-benzylamino)-pyridin-3-yl]-amide; N-[2-Dimethylamino-6-(4-fluoro-benzylamino)-pyridin-3-yl]-2-(3-fluoro-phenyl)-acetamide; N-[2-Dimethylamino-6-(4-fluoro-benzylamino)-pyridin-3-yl]-3,3-dimethyl-butyramide; 2-(3,5-Difluoro-phenyl)-N-[2-dimethylamino-6-(2,4,6-trimethyl-benzylamino)-pyridin-3-yl]-acetamide; 2-(3,5-Difluoro-phenyl)-N-[2-dimethylamino-6-(2,6-dimethyl-benzylamino)-pyridin-3-yl]-acetamide; N-[2-Dimethylamino-6-(5-fluoro-2-methyl-benzylamino)-pyridin-3-yl]-3,3-dimethyl-butyramide; or a stereoisomer or a mixture of its stereoisomers, or a pharmaceutically-acceptable addition salt thereof, or an N-oxide thereof.

### Formula 47

In an embodiment, the Kv7 channel activator may be selected from one of the following compounds according to Formula 47. Such compounds are described in US Publication No. US20110003867A1, published Jan. 6, 2011, and corresponding to U.S. application Ser. No. 12/747,422 filed Dec. 10, 2008; International Publication No. WO2009074594A1, published Jun. 18, 2009, and corresponding to International Application No. PCT/EP2008/067165 filed Dec. 10, 2008; which are incorporated by reference in their entirety herein. In the case of any conflict of terminology in the context of Formula 47, these references incorporated by reference herein control.

In an embodiment, the Kv7 channel activator is a compound according to Formula 47:

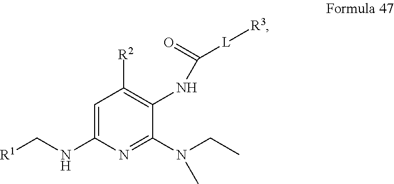

a stereoisomer or a mixture of its stereoisomers, or a pharmaceutically-acceptable addition salt thereof, or an N-oxide thereof, wherein R¹ represents alkyl or phenyl, which phenyl is optionally substituted one or more times with substituents selected from alkyl, halo and trifluoromethyl; R² represents hydrogen; R³ represents alkyl or phenyl, which phenyl is optionally substituted one or more times with substituents selected from alkyl, halo, alkoxy and trifluoromethyl; and L represents a linker selected from —CR′R″—, —CH₂—CR′R″—, —CR′R″—CH₂— and cycloalkyl, wherein R′ and R″, independently of each other, represent hydrogen, alkyl or halo.

In further embodiments, R¹ represents alkyl.

In further embodiments, R¹ represents phenyl, optionally substituted one or more times with substituents selected from alkyl, halo and trifluoromethyl.

In further embodiments, R³ represents phenyl, optionally substituted one or more times with substituents selected from alkyl, halo, alkoxy and trifluoromethyl.

In further embodiments, R³ represents alkyl.

In further embodiments, L represents a linker selected from —CR′R″—, —CH₂—CR′R″—, —CR′R″—CH₂— and cycloalkyl, wherein R′ and R″, independently of each other, represent hydrogen, alkyl or halo.

In further embodiments, the Kv7 Channel activator is selected from the group consisting of: N-[2-Dimethylamino-6-(4-fluoro-benzylamino)-pyridin-3-yl]-2-(3,5-difluoro-phenyl)-acetamide; N-[2-Ethylmethylamino-6-(4-fluoro-benzylamino)-pyridin-3-yl]-2-(3,4-difluoro-phenyl)-acetamide; N-[2-Ethylmethylamino-6-(4-fluoro-benzylamino)-pyridin-3-yl]-2-(3-fluoro-4-trifluoromethyl-phenyl)-acetamide; N-[2-Ethylmethylamino-6-(4-fluoro-benzylamino)-pyridin-3-yl]-3-(3-fluoro-phenyl)-propionamide; (R)—N-[2-(Ethyl-methyl-amino)-6-(4-fluoro-benzylamino)-pyridin-3-yl]-3-phenyl-butyramide; trans-2-Phenyl-cyclopropanecarboxylic acid [2-Ethylmethylamino-6-(4-fluoro-benzylamino)-pyridin-3-yl]-amide; N-[2-Ethylmethylamino-6-(4-fluoro-benzylamino)-pyridin-3-yl]-2-(3-fluoro-phenyl)-acetamide; N-[[2-Ethylmethylamino-6-(4-fluoro-benzylamino)-pyridin-3-yl]-3,3-dimethyl-butyramide; pyridin-3-yl]-acetamide; 2-(3,5-Difluoro-phenyl)-N-[6-(2,2-dimethyl-propylamino)-2-(ethyl-methyl-amino)-pyridin-3-yl]-acetamide; 2-(3,5-Difluoro-phenyl)-N-[2-(ethyl-methyl-amino)-6-isobutylamino-pyridin-3-yl]-acetamide; or a stereoisomer or a mixture of its stereoisomers, or a pharmaceutically-acceptable addition salt thereof, or an N-oxide thereof.

### Formula 48

In an embodiment, the Kv7 channel activator may be selected from one of the following compounds according to Formula 48. Such compounds are described in International Publication No. WO2007104717A1, published Sep. 20, 2007, and corresponding to International Application No. PCT/EP2007/052239 filed Mar. 9, 2007; US Publication No. US20090036473 A1, published Feb. 5, 2009, and corresponding to U.S. application Ser. No. 12/278,091 filed Mar. 9, 2007; which are incorporated by reference in their entirety herein. In the case of any conflict of terminology in the context of Formula 48, these references incorporated by reference herein control.

In an embodiment, the Kv7 channel activator is a compound according to Formula 48:

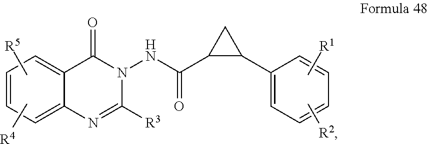

including any of its stereoisomers, or any mixture of its stereoisomers, or a pharmaceutically-acceptable addition salt thereof, or an N-oxide thereof, wherein R¹ and R², independently of each other, represent hydrogen, alkyl, cycloalkyl, halo, haloalkyl, hydroxy, alkoxy, baloalkoxy, amino, alkyl-carbonyl-amino, alkyl-sulfonyl, cyano or nitro; R³ represents alkyl, cycloalkyl or alkoxy; and R⁴ and R⁵, independently of each other, represent hydrogen, alkyl, cycloalkyl, halo, haloalkyl, hydroxy, alkoxy, haloalkoxy, amino, alkyl-carbonyl-amino, nitro or cyano.

In further embodiments, R¹ and R², independently of each other, represent hydrogen, alkyl, cycloalkyl, halo, haloalkyl, hydroxy, alkoxy, haloalkoxy, amino, alkyl-carbonyl-amino, alkyl-sulfonyl, cyano or nitro.

In further embodiments, R³ represents alkyl, cycloalkyl or alkoxy.

In further embodiments, R⁴ and R⁵, independently of each other, represent hydrogen, alkyd, cycloalkyl, halo, haloalkyl, hydroxy, alkoxy, haloalkoxy, amino, alkyl-carbonyl-amino, nitro or cyano.

In further embodiments, the Kv7 Channel activator is selected from the group consisting of: 2-(4-Chloro-phenyl)-cyclopropanecarboxylic acid (2-isopropyl-4-oxo-4H-quinazolin-3-yl)-amide; 2-(4-Fluoro-phenyl)-cyclopropanecarboxylic acid (2-isopropyl-4-oxo-4H-quinazolin-3-yl)-amide; 2-(3-Fluoro-4-methyl-phenyl)-cyclopropanecarboxylic acid (2-isopropyl-4-oxo-4H-quinazolin-3-yl)-amide; 2-Phenyl-cyclopropanecarboxylic acid (2-isopropyl-4-oxo-4H-quinazolin-3-yl)-amide; 2-(4-Chloro-phenyl)-cyclopropanecarboxylic acid (2-isopropyl-4-oxo-4H-quinazolin-3-yl)-amide; 2-(4-Fluoro-phenyl)-cyclopropanecarboxylic acid (2-isopropyl-4-oxo-4H-quinazolin-3-yl)-amide; 2-p-Tolyl-cyclopropanecarboxylic acid (2-isopropyl-4-oxo-4H-quinazolin-3-yl)-amide; 2-(3-Fluoro-phenyl)-cyclopropanecarboxylic acid (2-isopropyl-4-oxo-4H-quinazolin-3-yl)-amide; or 2-p-Tolyl-cyclopropanecarboxylic acid (2-isopropyl-4-oxo-4H-quinazolin-3-yl)-amide; or a pharmaceutically-acceptable addition salt thereof.

### Formula 49

In an embodiment, the Kv7 channel activator may be selected from one of the following compounds according to Formula 49. Such compounds are described in International Publication No. WO2007057447A1, published May 24, 2007, and corresponding to International Application No. PCT/EP2006/068627 filed Nov. 17, 2006; US Publication No. US20090291973 A1, published Nov. 26, 2009, and corresponding to U.S. application Ser. No. 12/085,188 filed Nov. 17, 2006; which are incorporated by reference in their entirety herein. In the case of any conflict of terminology in the context of Formula 49, these references incorporated by reference herein control.

In an embodiment, the Kv7 channel activator is a compound according to Formula 49:

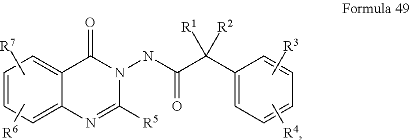

including any of its stereoenantiomers or any mixture of its stereoenantiomers, or a pharmaceutically-acceptable addition salt thereof, or an N-oxide thereof, wherein R¹ represents hydrogen or alkyl; and R² represents alkyl, cycloalkyl, halo, haloalkyl, hydroxyalkyl, hydroxy, alkoxy, phenyl, phenylalkyl, amino, alkyl-carbonyl-amino, cyano or nitro; or R¹ and R² together with the carbon atom to which they are attached form a cycloalkyl group; or R¹ represents hydrogen; and R² together with R³ attached in ortho-position on the aromatic ring form a —(CH₂)ₙ— bridge, wherein n is 1, 2 or 3; R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or R³ and R⁴ together form a methylenedioxy group; or R³ attached in ortho-position on the aromatic ring and together with R² form a —(CH₂)ₙ— bridge, wherein n is 1, 2 or 3; and R⁴ is as defined above; R⁵ represents alkyl, cycloalkyl, alkoxy, alkylthio or phenyl; and R⁶ and R⁷, independently of each other, represent hydrogen, alkyl, cycloalkyl, halo, haloalkyl, hydroxy, alkoxy, haloalkoxy, amino, alkyl-carbonyl-amino (acetamido), nitro, cyano or phenyl; provided, however, that if R¹ is hydrogen, R² is methyl, R³ and R⁴ represent hydrogen, R⁵ is isopropyl, and R⁶ and R⁷ represent hydrogen, then the compound it is not a quinazoline derivative racemate but the R- or S-enantiomer of the quinazoline derivative.

In further embodiments, R¹ represents hydrogen or alkyl.

In further embodiments, R² represents alkyl, cycloalkyl, halo, haloalkyl, hydroxyalkyl, hydroxy, alkoxy, phenyl, phenylalkyl, amino, alkyl-carbonyl-amino, cyano or nitro.

In further embodiments, R¹ represents hydrogen or methyl; and R² represents methyl, ethyl, isopropyl, cyclohexyl, fluoro or benzyl.

In further embodiments, R¹ and R² together with the carbon atom to which they are attached form a cycloalkyl group.

In further embodiments, R¹ represents hydrogen; and R² together with R³ attached in ortho-position on the aromatic ring form a —(CH₂)ₙ— bridge, wherein n is 1, 2 or 3.

In further embodiments, R³ and R⁴, independently of each other, represent hydrogen, alkyl, cycloalkyl, halo, haloalkyl, hydroxy, alkoxy, haloalkoxy, amino, alkyl-carbonyl-amino, alkyl-sulfonyl, phenyl, benzoyl, cyano or nitro; or R³ and R⁴ together form a methylenedioxy group.

In further embodiments, R³ attached in ortho-position on the aromatic ring and together with R² form a —(CH₂)ₙ— bridge, wherein n is 1, 2 or 3; and R⁴ is as defined in claim 7.

In further embodiments, R⁵ represents alkyl, cycloalkyl, alkoxy, alkylthio or phenyl.

In further embodiments, R⁶ and R⁷, independently of each other, represent hydrogen, alkyl, cycloalkyl, halo, haloalkyl, hydroxy, alkoxy, haloalkoxy, amino, alkyl-carbonyl-amino (acetamido), nitro, cyano or phenyl.

In further embodiments, the Kv7 Channel activator is selected from the group consisting of: N-(2-Isopropyl-4-oxo-4H-quinazolin-3-yl)-3-methyl-2-phenyl-butyramide; 2-(4-Chloro-phenyl)-N-(2-isopropylsulfanyl-4-oxo-4H-quinazolin-3-yl)-butyramide; 2-(3,5-Difluoro-phenyl)-N-(2-isopropylsulfanyl-4-oxo-7-trifluoromethyl-4H-quinazolin-3-yl)-propionamide; N-(2-Ethyl-7-fluoro-4-oxo-4H-quinazolin-3-yl)-2-phenyl-propionamide; (S)—N-(2-Isopropyl-4-oxo-4H-quinazolin-3-yl)-2-phenyl-propionamide; N-(2-Isopropyl-4-oxo-4H-quinazolin-3-yl)-2,3-diphenyl-propionamide; Bicyclo[4.2.0]octa-1,3,5-triene-7-carboxylic acid (2-isopropyl-4-oxo-4H-quinazolin-3-yl)-amide; N-(2-Isopropyl-4-oxo-4H-quinazolin-3-yl)-2-p-tolyl-propionamide; 2-Cyclohexyl-N-(2-isopropyl-4-oxo-4H-quinazolin-3-yl)-2-phenyl-acetamide; 2-(3-Benzoyl-phenyl)-N-(2-isopropyl-4-oxo-4H-quinazolin-3-yl)-propionamide; 1-Phenyl-cyclopropanecarboxylic acid (2-isopropyl-4-oxo-4H-quinazolin-3-yl)-amide; 2-(3,4-Dimethoxy-phenyl)-N-(2-isopropyl-4-oxo-4H-quinazolin-3-yl)-propionamide; (R)—N-(2-Isopropyl-4-oxo-4H-quinazolin-3-yl)-2-phenyl-propionamide; N-(2-Isopropyl-4-oxo-4H-quinazolin-3-yl)-2-phenyl-isobutyramide; 2-(4-Chloro-phenyl)-N-(2-isopropyl-4-oxo-7-trifluoromethyl-4H-quinazolin-3-yl)-propionamide; N-(2-Isopropyl-4-oxo-4H-quinazolin-3-yl)-2-(4-trifluoromethyl-phenyl)-propionamide; 2-(3,4-Dichloro-phenyl)-N-(2-isopropyl-4-oxo-7-trifluoromethyl-4H-quinazolin-3-yl)-propionamide; 2-(3-Fluoro-phenyl)-N-(2-isopropyl-4-oxo-4H-quinazolin-3-yl)-propionamide; 2-(4-Chloro-phenyl)-N-(2-isopropyl-4-oxo-4H-quinazolin-3-yl)-propionamide; N-(2-Isopropyl-4-oxo-7-trifluoromethyl-4H-quinazolin-3-yl)-2-(4-trifluoromethyl-phenyl)-propionamide; N-(2-Isopropyl-4-oxo-4H-quinazolin-3-yl)-2-(4-methoxy-phenyl)-propionamide; N-(2-Isopropyl-4-oxo-7-trifluoromethyl-4H-quinazolin-3-yl)-2-(4-methoxy-phenyl)-propionamide; 2-(3,4-Dichloro-phenyl)-N-(2-isopropyl-4-oxo-4H-quinazolin-3-yl)-propionamide; 2-(3-Fluoro-4-methyl-phenyl)-N-(2-isopropyl-4-oxo-7-trifluoromethyl-4H-quinazolin-3-yl)-propionamide; 2-(3-Fluoro-4-methyl-phenyl)-N-(2-isopropyl-4-oxo-4H-quinazolin-3-yl)-propionamide; 2-(4-Isobutyl-phenyl)-N-(2-isopropyl-4-oxo-4H-quinazolin-3-yl)-propionamide; N-(7-Chloro-2-isopropyl-4-oxo-4H-quinazolin-3-yl)-2-(3-fluoro-phenyl)-propionamide; N-(6-Chloro-2-isopropyl-4-oxo-4H-quinazolin-3-yl)-2-(3-fluoro-phenyl)-propionamide; 2-(4-Fluoro-phenyl)-N-(2-isopropyl-4-oxo-4H-quinazolin-3-yl)-propionamide; N-(5-Chloro-2-isopropyl-4-oxo-4H-quinazolin-3-yl)-2-(3-fluoro-phenyl)-propionamide; N-(8-Chloro-2-isopropyl-4-oxo-4H-quinazolin-3-yl)-2-(3-fluoro-phenyl)-propionamide; N-(8-Cyano-2-isopropyl-4-oxo-4H-quinazolin-3-yl)-2-(3-fluoro-phenyl)-propionamide; 7-Methyl-bicyclo[4.2.0]octa-1 (6),2,4-triene-7-carboxylic acid (2-isopropyl-4-oxo-4H-quinazolin-3-yl)-amide; N-(2-Isopropylsulfanyl-4-oxo-4H-quinazolin-3-yl)-2-phenyl-propionamide; 2-(4-Chloro-phenyl)-N-(2-isopropylsulfanyl-4-oxo-4H-quinazolin-3-yl)-propionamide; 2-(4-Chloro-phenyl)-N-(2-ethoxy-4-oxo-4H-quinazolin-3-yl)-propionamide; 2-(3,5-Difluoro-phenyl)-N-(2-methylsulfanyl-4-oxo-7-trifluoromethyl-4H-quinazolin-3-yl)-propionamide; 2-Fluoro-N-(2-isopropyl-4-oxo-4H-quinazolin-3-yl)-2-phenyl-propionamide; (S)-2-Fluoro-N-(2-isopropyl-4-oxo-4H-quinazolin-3-yl)-2-phenyl-propionamide; (R)-2-Fluoro-N-(2-isopropyl-4-oxo-4H-quinazolin-3-yl)-2-phenyl-propionamide; (S)-2-Fluoro-N-(2-isopropyl-4-oxo-7-trifluoromethyl-4H-quinazolin-3-yl)-2-phenyl-propionamide; or (R)-2-Fluoro-N-(2-isopropyl-4-oxo-7-trifluoromethyl-4H-quinazolin-3-yl)-2-phenyl-propionamide; or an N-oxide thereof, any of its stereoenantiomers or any mixture of its stereoenantiomers, or a pharmaceutically-acceptable addition salt thereof.

### Formula 50

In an embodiment, the Kv7 channel activator may be selected from one of the following compounds according to Formula 50. Such compounds are described in U.S. Pat. No. 7,741,352, issued Jun. 22, 2010, and corresponding to U.S. application Ser. No. 10/546,533 filed Mar. 11, 2004; International Publication No. WO2004080377A2, published Sep. 23, 2004, and corresponding to International Application No. PCT/EP2004/050290 filed Mar. 11, 2004; which are incorporated by reference in their entirety herein. In the case of any conflict of terminology in the context of Formula 50, these references incorporated by reference herein control.

In an embodiment, the Kv7 channel activator is a compound according to Formula 50:

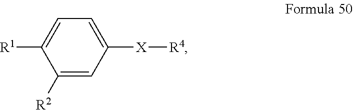

any of its enantiomers or any mixture of its enantiomers, or a prodrug, or a pharmaceutically-acceptable addition salt thereof, wherein R¹ represents —CN; R² represents halo, haloalkyl, hydroxyl or alkoxy; X represents, NR″ or NR″CH₂ (read in the stated direction); wherein R″ represents hydrogen; R⁴ represents aryl-alkyl, which is substituted one or more times with substituents selected from the group consisting of halo, or methylenedioxy; or R⁴ represents a group of formula —Z′—L″—Z″; wherein Z′ and Z″, independently of one another, represent an aryl group, which aryl may be optionally substituted one or more times with halo; and L″ represents a single (covalent) bond, or a linker selected from O or OCH₂, with the proviso that when X represents NR″, then R⁴ represents aryl-alkyl and when X represents NR″CH₂, then R⁴ represents a group of formula —Z′—L″—Z″.

In further embodiments, R⁴ represents benzyl which is substituted one or two times with halo or one time with methylenedioxy.

In further embodiments, R⁴ represents 4-fluoro-benzyl, 3,4-dichloro-benzyl or benzo[1,3]dioxol-5-ylmethyl.

In further embodiments, the Kv7 Channel activator is selected from the group consisting of: 4-(4-Fluoro-benzylamino)-2-hydroxy-benzonitrile 4-(3,4-Dichloro-benzylamino)-2-hydroxy-benzonitrile or 4-[(Benzo[1,3]dioxol-5-ylmethyl)-amino]-2-hydroxy-benzonitrile; or a pharmaceutically-acceptable addition salt thereof.

In further embodiments, R⁴ represents a group of formula —Z′—L″—Z″; wherein Z′ represents phenyl, or phen-4-yl; which phenyl may optionally be substituted one or two times with halo; and Z″ represent phenyl; which may optionally be substituted one or two times with halo; and L″ represents a single (covalent) bond, or a linker selected from alkyl, O, or OCH₂.

In further embodiments, R⁴ represents 3-phenoxy-phenyl, 3-benzyloxy-phenyl, or biphenyl-4-yl.

In further embodiments, the Kv7 Channel activator is selected from the group consisting of: 2-Hydroxy-4-(3-phenoxy-benzylamino)-benzonitrile; 4-(3-Benzyloxy-benzylamino)-2-hydroxy-benzonitrile; or 4-[(Biphenyl-4-ylmethyl)-amino]-2-hydroxy-benzonitrile; or a pharmaceutically-acceptable addition salt thereof.

### Formula 51

In an embodiment, the Kv7 channel activator may be selected from one of the following compounds according to Formula 51. Such compounds are described in U.S. Pat. No. 7,741,352, issued Jun. 22, 2010, and corresponding to U.S. application Ser. No. 10/546,533 filed Mar. 11, 2004; International Publication No. WO2004080377A2, published Sep. 23, 2004 and corresponding to International Application No. PCT/EP2004/050290 filed Mar. 11, 2004; which are incorporated by reference in their entirety herein. In the case of any conflict of terminology in the context of Formula 51, these references incorporated by reference herein control.

In an embodiment, the Kv7 channel activator is a compound according to Formula 51:

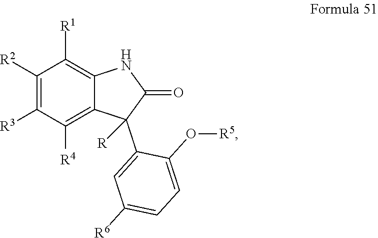

wherein, R represents hydrogen, halogen or hydroxy. R¹, R², R³ and R⁴ independently of each another represent hydrogen, halogen, alkyl, trihalogenmethyl, phenyl, p-methyl-phenyl or p-trihalogenmethyl-phenyl, or R¹ and R², R² and R³, or R³ and R⁴ are joined together to form a benzo fused ring, R⁵ represents hydrogen or alkyl, and R⁶ represents halogen or trihalogenmethyl, or a pharmaceutically-acceptable addition salt thereof.

In further embodiments, R represents hydrogen, halogen or hydroxy, R¹, R², R³ and R⁴ independently of each another represent hydrogen, halogen, alkyl, trihalogenmethyl, phenyl, p-methyl-phenyl or p-trihalogenmethyl-phenyl, and when R¹ and R⁴ are hydrogen, then R² or R³ is phenyl, p-methyl-phenyl or p-trihalogenmethyl-phenyl; or R¹ and R², R² and R³, or R³ and R⁴ are joined together to form a benzo fused ring, R⁵ represents hydrogen or alkyl, and R⁶ represents halogen or trihalogenmethyl.

In further embodiments, R¹, R³ and R⁴ represent hydrogen, and R² represents halogen, trihalogenmethyl or phenyl.

In further embodiments, R¹ and R², R² and R³, or R³ and R⁴ are joined together to form a benzo fused ring.

In further embodiments, R⁵ is hydrogen or methyl.

In further embodiments, R⁶ is chlorine.

In further embodiments, the Kv7 Channel activator is selected from the group consisting of: (+)-3-(5-chloro-2-methoxyphenyl)-1,3-dihydro-6-(trifluoromethyl)-2H-indol-2-one; (−)-3-(5-chloro-2-methoxyphenyl)-1,3-dihydro-6-(trifluoromethyl)-2H-indol-2-one; (±)-3-(5-chloro-2-methoxyphenyl)-1,3-dihydro-6 (4-methylphenyl)-2H-indol-2-one; (±)-3-(5-chloro-2-hydroxyphenyl)-1,3-dihydro-2H-indol-one; (±)-3-(5-chloro-2-hydroxyphenyl)-4,6-dichloro-1,3-dihydro-2H-indol-one; (±)-3-(5-chloro-2-hydroxyphenyl)-1,3-dihydro-6-phenyl-2H-indol-one; (±)-3-(5-chloro-2-hydroxyphenyl)-1,3-dihydro-6-iodo-2H-indol-one; (±)-3-(5-chloro-2-hydroxyphenyl)-1,3-dihydro-6-trifluoromethyl)-2H-indol-2-one; (±)-3-(5-chloro-2-hydroxyphenyl)-1,3-dihydro-6-[4-(trifluoromethyl)-phenyl]-2H-indol-2-one; (±)-3-(5-chloro-2-hydroxyphenyl)-1,3-dihydro-2H-benz[e]indol-2-one; (±)-3-(5-chloro-2-hydroxyphenyl)-1,3-dihydro-2H-benz[f]indol-2-one; (±)-3-(5-chloro-2-hydroxyphenyl)-1,3-dihydro-2H-benx[g]indol-2-one; (±)-3-(5-chloro-2-methoxyphenyl)-1,3-dihydro-3-hydroxy-6-(trifluoromethyl)-2H-indol-2-one; (±)-3-(5-chloro-2-methoxyphenyl)-1,3-dihydro-3-hydroxy-4-(trifluoromethyl)-2H-indol-2-one; (±)-3-(5-chloro-2-methoxyphenyl)-1,3-dihydro-3-hydroxy-6-(trifluoromethyl)-2H-indol-2-one; (−)-3-(5-chloro-2-methoxyphenyl)-1,3-dihydro-hydroxy-6-(trifluoromethyl)-2H-indol-2-one; (±)-3-(5-chloro-2-methoxyphenyl)-1,3-dihydro-3-hydroxy-7-(trifluoromethyl)-2H-indol-2-one; (±)-3-(5-chloro-2-methoxyphenyl)-1,3-dihydro-3-hydroxy-5-bromo-2H-indol-2-one; (±)-3-(5-chloro-2-methoxyphenyl)-1,3-dihydro-3-hydroxy-6-iodo-2H-indol-2-one; (±)-3-(5-chloro-2-methoxyphenyl)-4,6-dichloro-1,3-dihydro-3-hydroxy-2H-indol-2-one; (±)-3-(5-chloro-2-methoxyphenyl)-1,3-dihydro-3-hydroxy-2H-benz[f]indol-2-one; (±)-3-(5-chloro-2-hydroxyphenyl)-1,3-dihydro-3-hydroxy-4,6-bis-(trifluoromethyl)-2H-indol-2-one; (±)-3-(5-chloro-2-hydroxyphenyl)-1,3-dihydro-3-hydroxy-4-(trifluoromethyl)-2H-indol-2-one; (±)-3-(5-chloro-2-hydroxyphenyl)-1,3-dihydro-3-hydroxy-6-(trifluoromethyl)-2H-indol-2-one; (±)-3-(5-chloro-2-hydroxyphenyl)-1,3-dihydro-3-hydroxy-6-(trifluoromethyl)-2H-indol-2-one; (−)-3-(5-chloro-2-hydroxyphenyl)-1,3-dihydro-3-hydroxy-6-(trifluoromethyl)-2H-indol-2-one; (±)-3-(5-chloro-2-hydroxyphenyl)-1,3-dihydro-3-hydroxy-7-(trifluoromethyl)-2H-indol-2-one; (±)-3-(5-chloro-2-hydroxyphenyl)-4,6-dichloro-1,3-dihydro-3-hydroxy-2H-indol-2-one; (±)-3-(5-chloro-2-hydroxyphenyl)-1,3-dihydro-3-hydroxy-5-bromo-2H-indol-2-one; (±)-3-(5-chloro-2-hydroxyphenyl)-1,3-dihydro-3-hydroxy-6-iodo-2H-indol-2-one; (±)-1,3-dihydro-3-hydroxy-3-[2-hydroxy-5-(trifluoromethyl)-phenyl]-6-(trifluoromethyl)-2H-indol-2-one; (±)-3-(5-chloro-2-hydroxyphenyl)-1,3-dihydro-3-hydroxy-2H-benz[g]indol-2-one; (±)-3-(5-chloro-2-hydroxyphenyl)-1,3-dihydro-3-hydroxy-2H-benz[f]indol-2-one; (±)-3-(5-chloro-2-hydroxyphenyl)-1,3-dihydro-3-fluoro-6-(trifluoromethyl)-2H-indol-2-one; (3S)-(±)-(5-chloro-2-methoxyphenyl)-1,3-dihydro-3-fluoro-6-(trifluoromethyl)-2H-indol-2-one; (3R)-(−)-(5-chloro-2-methoxyphenyl)-1,3-dihydro-3-fluoro-6-(trifluoromethyl)-2H-indol2-one; (±)-3-(5-chloro-2-methoxyphenyl)-1,3-dihydro-3-fluoro-7-(trifluoromethyl)2H-indol-2-one; (±)-3-(5-chloro-2-methoxyphenyl)-1,3-dihydro-3-fluoro-6-phenyl-2H-indol-2-one; (±)-3-(5-chloro-2-methoxyphenyl)-1,3-dihydro-3-fluoro-6-iodo-2H-indol-2-one; (±)-3-(5-chloro-2-methoxyphenyl)-1,3-dihydro-3-fluoro-5-methyl-2H-indol-2-one; (±)-3-(5-chloro-2-methoxyphenyl)-1,3-dihydro-3-fluoro-5-bromo-2H-indol-2-one; (±)-3-(5-chloro-2-methoxyphenyl)-1,3-dihydro-3-fluoro-4,6-bis-(trifluoromethyl)-2H-indol-2-one; or (±)-3-(5-chloro-2-methoxyphenyl)-1,3-dihydro-3-fluoro-7-(trifluoromethyl)-2H-indol-2-one; or a pharmaceutically-acceptable addition salt thereof.

In further embodiments, R represents hydrogen, halogen or hydroxy, R¹, R², R³ and R⁴ independently of each another represent hydrogen, halogen, alkyl, trihalogenmethyl, phenyl, p-methyl-phenyl or p-trihalogenmethyl-phenyl, or R¹ and R², R² and R³, or R³ and R⁴ are joined together to form a bezo fused ring, R⁵ represents hydrogen or alkyl, and R⁶ represents halogen or trihalogenmethyl, or a pharmaceutically-acceptable addition salt thereof, together with at least one pharmaceutically-acceptable carrier or diluent.

### Formula 52

In another embodiment, the Kv7 channel activator may be selected from one of the following compounds according to Formula 52. Such compounds are described in International Publication No. CN114380731A, published Apr. 22, 2022, and corresponding to International Application No. CN202210226122.7A filed Mar. 9, 2022; which is incorporated by reference in its entirety herein. In the case of any conflict of terminology in the context of Formula 52, this reference incorporated by reference herein controls.

In an embodiment, the Kv7 channel activator is a compound according to Formula 52:

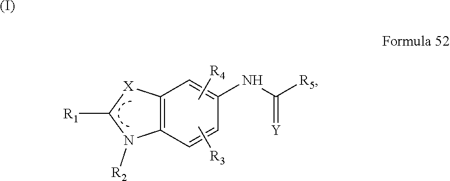

wherein, R₁ is selected from H, halogen, substituted or unsubstituted phenyl, or a substituted or unsubstituted phenylalkyl group, the substituents of said phenyl and phenylalkyl groups each being independently selected from halogen or haloalkyl; X is selected from S or C; R₂ is optionally selected from H, alkyl, alkenyl or alkynyl; R₃ and R₄ each independently selected from H or alkyl; y is selected from O or S; R₅ is selected from substituted or unsubstituted alkyl, substituted or unsubstituted cycloalkyl, alkoxy or furyl, the substituent of the alkyl is selected from alkoxy, dialkylamino or alkoxycarbonyl, and the substituent of the cycloalkyl is selected from halogen.

In further embodiments, R₂ is selected from H, C₁-C₃Alkyl radical, C₁-C₃Alkenyl or C₁-C₃Alkynyl.

In further embodiments, R₃ and R₄ are each independently selected from H or C₁-C₆An alkyl group.

In further embodiments, R₅ is selected from substituted or unsubstituted C₁-C₆Alkyl, substituted or unsubstituted C₃-C₆Cycloalkyl radical, C₁-C₆Alkoxy or furyl, the substituents of the alkyl or the alkoxy being selected from C₁-C₆Alkoxy, di(C)₁-C₄Alkyl)amino or C₁-C₆Alkoxycarbonyl, the substituents of cycloalkyl being selected from halogen.

In further embodiments, the formula is one of formulas II-IV:

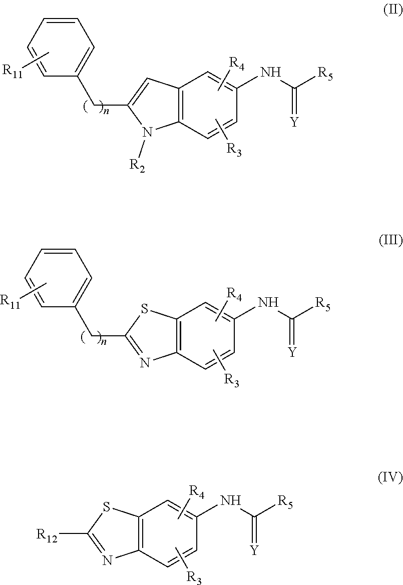

wherein, n is more than or equal to 0; R₁₁ and R₁₂ each independently selected from H, halogen or halomethyl; R₂ is selected from H, C₁-C₃Alkyl radical, C₂-C₃Alkenyl or C₂-C₃An alkynyl group; R₃ and R₄ are independently selected from H or C₁-C₆ alkyl group; y is selected from O or S; R₅ is selected from substituted or unsubstituted C₁-C₆Alkyl, substituted or unsubstituted C₃-C₆Cycloalkyl radical, C₁-C₆Alkoxy or furyl, the substituents of the alkyl being selected from C₁-C₆Alkoxy, di(C)₁-C₄Alkyl)amino or C₁-C₆Alkoxycarbonyl, the substituents of cycloalkyl being selected from halogen.

In further embodiments, n is 0 to 3, R₁₁ is selected from H, F or trifluoromethyl, and R₁₂ is selected from H, F, Cl or methyl.

In further embodiments, R₃ is one of H or methyl, and R₄ is other of H or methyl.

In further embodiments, R₅ is selected from methyl, ethyl, isopropyl, isobutyl, neopentyl, cyclobutyl, ethoxy, isopropoxy, tert-butoxy or tetrahydrofuranyl.

### Formula 53

In an embodiment, the Kv7 channel activator may be selected from one of the following compounds according to Formula 53. Such compounds are described in International Publication No. WO2021260090A1, published Dec. 30, 2021, and corresponding to International Application No. PCT/EP2021/067288 filed Jun. 24, 2021; which is incorporated by reference in its entirety herein. In the case of any conflict of terminology in the context of Formula 53, this reference incorporated by reference herein controls.

In an embodiment, the Kv7 channel activator is a compound according to Formula 53:

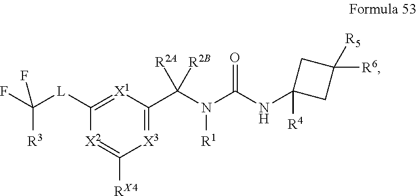

wherein, X¹ represents nitrogen or CR^{X1}; wherein R^{X1} represents hydrogen, halogen, (C₁₋₄)alkyl, or (C₁₋₄)alkoxy; X² represents nitrogen or CR^{X2}; wherein R^{X2} represents hydrogen, halogen, (C₁₋₄)alkyl, or (C₁₋₄)alkoxy; X³ represents nitrogen or CR^{X3}; wherein R^{X3} represents hydrogen, halogen, (C₁₋₄)alkyl, (C₁₋₄)alkoxy, or hydroxy; R¹ represents hydrogen or methyl; R^{X4} represents hydrogen, halogen, or (C₁₋₄)alkyl; ⋅ R^{2A} represents hydrogen; (C₁₋₄)alkyl; (C₂₋₄)alkenyl; (C₂₋₄)alkynyl; (C₃₋₆)cycloalkyl; (C₁₋₄)fluoroalkyl; (C₁₋₄)hydroxyalkyl; (C₁₋₄)alkoxy-(C₁₋₂)alkyl; (C₁₋₂)alkoxy-(C₁₋₂)alkoxy-(C₁₋₂)alkyl; (C₁₋₂)alkyl-S—(C₁₋₂)alkyl; (C₁₋₂)alkyl-(SO₂)—(C₁₋₂)alkyl; cyano; (C₁₋₂) cyanoalkyl; H₂N—C(O)—(C₁₋₂)alkyl; (R^{N1})₂N—(C₁₋₂)alkyl or (R^{N1})₂N—C(O)—, wherein R^{N1} independently represents hydrogen or (C₁₋₂)alkyl; or a 5-membered heteroaryl group containing one to four nitrogen atoms, wherein said 5-membered heteroaryl group is independently unsubstituted or mono-substituted with (C₁₋₄)alkyl; and R^{2B} represents hydrogen or methyl; or R^{2A} and R^{2B} form, together with the carbon atom to which they are attached, a ring of 3- to 6 members, wherein the members needed to complete said ring are each independently selected from —CH₂— and —O— and wherein said ring does not contain more than one-O-member; L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, (C₁₋₄)alkyl, CH₃—O—CH₂—, or (CH₃)₂NCH₂—; wherein the asterisks indicate the bond which is linked to the aromatic carbon atom; R³ represents hydrogen or fluoro; ⋅ R⁴ represents hydrogen or (C₁₋₄)alkyl; R⁵ represents hydrogen, fluoro, or hydroxy; and R⁶ represents fluoro or (C₁)fluoroalkyl; or ⋅ R⁴ and R⁵ together represent a bridge selected from —CH₂— and —CH₂CH₂—; and R⁶ represents hydrogen, fluoro, (C₁)fluoroalkyl, or (C₁-4)alkyl; or a salt thereof.

In further embodiments, X¹ represents CR^{X1}; wherein R^{X1} represents hydrogen or halogen; X² represents nitrogen or CH; X³ represents nitrogen or CH; R¹ represents hydrogen; R^{X4} represents hydrogen, halogen, or (C₁₋₄)alkyl; R^{2A} represents hydrogen; (C₁₋₄)alkyl; (C₁₋₄)fluoroalkyl; (C₁₋₄)hydroxyalkyl; or (C₁₋₄)alkoxy-(C₁₋₂)alkyl; R^{2B} represents hydrogen; L represents a direct bond, —CH₂—O—*, or —O—; wherein the asterisk indicates the bond which is linked to the aromatic carbon atom; R³ represents hydrogen or fluoro; ⋅ R⁴ represents hydrogen or (C₁₋₄)alkyl; R⁵ represents hydrogen, fluoro, or hydroxy; and R⁶ represents fluoro or (C₁)fluoroalkyl; or ⋅ R⁴ and R⁵ together represent a bridge selected from —CH₂— and —CH₂CH₂—; and R⁶ represents hydrogen, fluoro, (C₁)fluoroalkyl, or (C₁-4)alkyl; or a salt thereof (meaning a salt of the compound of Formula 53).

In further embodiments, R^{2A} represents hydrogen, (C₁₋₄)alkyl, (C₁₋₄)fluoroalkyl, (C₁-4)hydroxyalkyl, or methoxymethyl; and R^{2B} represents hydrogen; or a salt thereof.

In further embodiments, L represents a direct bond; or a salt thereof.

In further embodiments, R³ represents fluoro; or a salt thereof.

In further embodiments, R^{X4} represents hydrogen; or a salt thereof.

In further embodiments, each of X¹, X², and X³ represents CH; or a salt thereof.

In further embodiments, the fragment

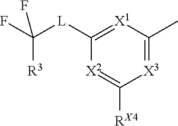

represents:

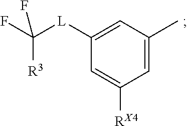

wherein R^{X4} represents hydrogen or halogen; R³ represents hydrogen or fluoro; and L represents a direct bond, —CH₂—O—*, or —O—; wherein the asterisk indicates the bond which is linked to the aromatic carbon atom; or; wherein X³ represents nitrogen or CH; R^{X4} represents hydrogen or (C₁₋₄)alkyl; R³ represents hydrogen or fluoro; and L represents —CH₂—O—*, or —O—; wherein the asterisk indicates the bond which is linked to the aromatic carbon atom; or a salt thereof.

In further embodiments, R⁴ represents hydrogen; R⁵ represents hydrogen or fluoro; and R⁶ represents fluoro, difluoromethyl or trifluoromethyl; or a salt thereof.

In further embodiments, R⁴ and R⁵ together represent a —CH₂— bridge; and R⁶ represents hydrogen, fluoro, difluoromethyl, or trifluoromethyl; or a salt thereof.

In further embodiments, the Kv7 Channel activator is selected from the group consisting of: 1-(3,3-Difluoro-cyclobutyl)-3-(3-trifluoromethyl-benzyl)-urea; 1-Bicyclo[1.1.1]pent-1-yl-3-[1-(3-trifluoromethyl-phenyl)-ethyl]-urea; 1-(3-Difluoromethyl-cyclobutyl)-3-[1-(3-trifluoromethyl-phenyl)-ethyl]-urea; 1-(3-Fluoro-bicyclo[1.1.1]pent-1-yl)-3-[1-(3-trifluoromethyl-phenyl)-ethyl]-urea; 1-(3-Difluoromethyl-bicyclo[1.1.1]pent-1-yl)-3-[1-(3-trifluoromethyl-phenyl)-ethyl]-urea; 1-Bicyclo[1.1.1]pent-1-yl-3-[1-(3-trifluoromethoxy-phenyl)-ethyl]-urea; 1-(3-Difluoromethyl-cyclobutyl)-3-[1-(3-trifluoromethoxy-phenyl)-ethyl]-urea; 1-(3-Fluoro-bicyclo[1.1.1]pent-1-yl)-3-[1-(3-trifluoromethoxy-phenyl)-ethyl]-urea; 1-[2,2-Difluoro-1-(3-trifluoromethyl-phenyl)-ethyl]-3-(3-hydroxy-3-trifluoromethyl-cyclobutyl)-urea; 1-(3,3-Difluoro-1-methyl-cyclobutyl)-3-[2,2-difluoro-1-(3-trifluoromethyl-phenyl)-ethyl]-urea; 1-Bicyclo[1.1.1]pent-1-yl-3-[1-(3-difluoromethoxy-phenyl)-ethyl]-urea; 1-(3-Difluoromethyl-bicyclo[1.1.1]pent-1-yl)-3-[2-hydroxy-1-(3-trifluoromethyl-phenyl)-ethyl]-urea; 1-(3-Difluoromethyl-bicyclo[1.1.1]pent-1-yl)-3-[2,2-difluoro-1-(3-trifluoromethyl-phenyl)-ethyl]-urea; 1-(3-Difluoromethyl-bicyclo[1.1.1]pent-1-yl)-3-[2-methoxy-1-(3-trifluoromethyl-phenyl)-ethyl]-urea; 1-(3-Difluoromethyl-bicyclo[1.1.1]pent-1-yl)-3-(2-fluoro-3-trifluoromethyl-benzyl)-urea; 1-(3-Difluoromethyl-bicyclo[1.1.1]pent-1-yl)-3-(3-fluoro-5-trifluoromethyl-benzyl)-urea; 1-(3-Fluoro-bicyclo[1.1.1]pent-1-yl)-3-[3-(2,2,2-trifluoro-ethoxy)-benzyl]-urea; 1-(3-Difluoromethyl-cyclobutyl)-3-[3-(2,2,2-trifluoro-ethoxy)-benzyl]-urea; 1-[3-(2,2,2-Trifluoro-ethoxy)-benzyl]-3-(3-trifluoromethyl-bicyclo[1.1.1]pent-1-yl)-urea; 1-(3-Difluoromethoxy-benzyl)-3-(3-fluoro-bicyclo[1.1.1]pent-1-yl)-urea; 1-(3-Difluoromethoxy-benzyl)-3-(3-difluoromethyl-cyclobutyl)-urea; 1-(3-Difluoromethoxy-benzyl)-3-(3-trifluoromethyl-bicyclo[1.1.1]pent-1-yl)-urea; 1-(3-Trifluoromethoxy-benzyl)-3-(3-trifluoromethyl-bicyclo[1.1.1]pent-1-yl)-urea; 1-(3-Fluoro-bicyclo[1.1.1]pent-1-yl)-3-(3-trifluoromethoxy-benzyl)-urea; 1-(3-Difluoromethyl-cyclobutyl)-3-(3-trifluoromethoxy-benzyl)-urea; 1-Bicyclo[1.1.1]pent-1-yl-3-(3-trifluoromethoxy-benzyl)-urea; 1-(3-Difluoromethyl-benzyl)-3-(3-difluoromethyl-cyclobutyl)-urea; 1-(3-Difluoromethyl-benzyl)-3-(3-trifluoromethyl-bicyclo[1.1.1]pent-1-yl)-urea; 1-(3-Difluoromethyl-cyclobutyl)-3-(2-trifluoromethoxy-pyridin-4-ylmethyl)-urea; 1-(2-Trifluoromethoxy-pyridin-4-ylmethyl)-3-(3-trifluoromethyl-bicyclo[1.1.1]pent-1-yl)-urea; 1-(3-Difluoromethyl-cyclobutyl)-3-{2-methoxy-1-[2-(2,2,2-trifluoro-ethoxy)-pyridin-4-yl]-ethyl}-urea; 1-{2-Methoxy-1-[2-(2,2,2-trifluoro-ethoxy)-pyridin-4-yl]-ethyl}-3-(3-trifluoromethyl-cyclobutyl)-urea; 1-[1-(2-Difluoromethoxy-pyridin-4-yl)-ethyl]-3-(3-difluoromethyl-cyclobutyl)-urea; 1-{1-[2-Methyl-6-(2,2,2-trifluoro-ethoxy)-pyrimidin-4-yl]-ethyl}-3-(3-trifluoromethyl-bicyclo[1.1.1]pent-1-yl)-urea; 1-Bicyclo[1.1.1]pent-1-yl-3-(3-trifluoromethyl-benzyl)-urea; 1-(3-Fluoro-bicyclo[1.1.1]pent-1-yl)-3-(3-trifluoromethyl-benzyl)-urea; 1-(3-Trifluoromethyl-benzyl)-3-(3-trifluoromethyl-bicyclo[1.1.1]pent-1-yl)-urea; 1-(3-Difluoromethyl-cyclobutyl)-3-(3-trifluoromethyl-benzyl)-urea; 1-(3-Methyl-bicyclo[1.1.1]pent-1-yl)-3-(3-trifluoromethyl-benzyl)-urea; 1-(3-Fluoromethyl-bicyclo[1.1.1]pent-1-yl)-3-(3-trifluoromethyl-benzyl)-urea; 1-(3-Trifluoromethyl-benzyl)-3-(3-trifluoromethyl-cyclobutyl)-urea; 1-(3-Hydroxy-3-trifluoromethyl-cyclobutyl)-3-(3-trifluoromethyl-benzyl)-urea; 1-Bicyclo[1.1.1]pent-1-yl-3-[2-(2,2,2-trifluoro-ethoxy)-pyridin-4-ylmethyl]-urea; 1-(3-Fluoro-bicyclo[1.1.1]pent-1-yl)-3-[2-(2,2,2-trifluoro-ethoxy)-pyridin-4-ylmethyl]-urea; 1-[2-(2,2,2-Trifluoro-ethoxy)-pyridin-4-ylmethyl]-3-(3-trifluoromethyl-bicyclo[1.1.1]pent-1-yl)-urea; 1-(3-Difluoromethyl-cyclobutyl)-3-[2-(2,2,2-trifluoro-ethoxy)-pyridin-4-ylmethyl]-urea; 1-(3-Difluoromethyl-bicyclo[1.1.1]pent-1-yl)-3-(3-trifluoromethyl-benzyl)-urea; 1-Bicyclo[2.1.1]hex-1-yl-3-(3-trifluoromethyl-benzyl)-urea; 1-(3,3-Difluoro-1-methyl-cyclobutyl)-3-(3-trifluoromethyl-benzyl)-urea; 1-(3-(trifluoromethyl)benzyl)-3-((1s,3s)-3-(trifluoromethyl)cyclobutyl) urea; 1-(3-(trifluoromethyl)benzyl)-3-((1r,3r)-3-(trifluoromethyl)cyclobutyl) urea; 1-((1s,3s)-3-(difluoromethyl)cyclobutyl)-3-(3-(trifluoromethyl)benzyl) urea; 1-((1r,3r)-3-(difluoromethyl)cyclobutyl)-3-(3-(trifluoromethyl)benzyl) urea; 1-{(S)-1-[2-Methyl-6-(2,2,2-trifluoro-ethoxy)-pyrimidin-4-yl]-ethyl}-3-(3-trifluoromethyl-bicyclo[1.1.1]pent-1-yl)-urea; 1-((1r,3r)-3-(difluoromethyl)cyclobutyl)-3-((2-(2,2,2-trifluoroethoxy)pyridin-4-yl)methyl) urea; 1-((1s,3s)-3-(difluoromethyl)cyclobutyl)-3-((2-(2,2,2-trifluoroethoxy)pyridin-4-yl)methyl) urea; 1-((1s,3R)-3-(difluoromethyl)cyclobutyl)-3-((S)-1-(3-(trifluoromethoxy)phenyl)ethyl) urea; and 1-((1r,3S)-3-(difluoromethyl)cyclobutyl)-3-((S)-1-(3-(trifluoromethoxy)phenyl)ethyl) urea; or a salt thereof.

### Formula 54

In an embodiment, the Kv7 channel activator may be selected from one of the following compounds according to Formula 54. Such compounds are described in International Publication No. WO2021219594A1, published Nov. 4, 2021 and corresponding to International Application No. PCT/EP2021/060918 filed Apr. 27, 2021; which is incorporated by reference in its entirety herein. In the case of any conflict of terminology in the context of Formula 54, this reference incorporated by reference herein controls.

In an embodiment, the Kv7 channel activator is a compound according to Formula 54:

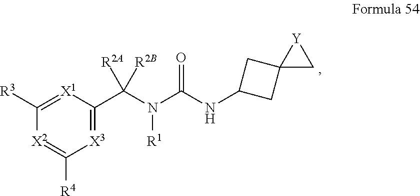

wherein, X¹ represents nitrogen or CR^{x1}, wherein R^{X1} represents hydrogen, halogen, (C₁₋₄)alkyl, or (C₁₋₄)alkoxy; X² represents nitrogen or CR^{X2}; wherein R^{X2} represents hydrogen, halogen, (C₁₋₄)alkyl, or (C₁₋₄)alkoxy; X³ represents nitrogen or CR^{X3}; wherein R^{X3} represents hydrogen, halogen, (C₁₋₄)alkyl, (C₁₋₄)alkoxy, or hydroxy; ⋅ R¹ represents hydrogen, or methyl; or ⋅ R¹ and R^{X1} together represent a —CH₂CH₂— bridge; Y represents —C(R^{Y1})(R^{Y2})—, or *—CH₂—C(R^{Y3})(R^{Y4})—, wherein the asterisk indicates the bond which is linked to the cyclobutyl ring; R^{Y1} represents hydrogen, or fluoro; R^{Y2} represents hydrogen or fluoro; R¹³ represents hydrogen, fluoro or iodo; R^{Y4} represents hydrogen or fluoro; R^{2A} represents hydrogen; (C₁₋₄)alkyl; (C₂₋₄)alkenyl; (C₂₋₄)alkynyl; (C₃₋₆)cycloalkyl; (C₁₋₄)fluoroalkyl; (C₁₋₄)hydroxyalkyl; (C₁₋₄)alkoxy-(C₁₋₂)alkyl; (C₁₋₂)alkoxy-(C₁₋₂)alkoxy-(C₁₋₂)alkyl; (C₁₋₂)alkyl-S—(C₁₋₂)alkyl; (C₁₋₂)alkyl-(SO₂)—(C₁₋₂)alkyl; cyano; (C₁₋₂) cyanoalkyl; H₂N—C(O)—(C₁₋₂)alkyl; (R^{N1})₂N—(C₁₋₂)alkyl or (R^{N1})₂N—C(O)—, wherein R^{N1} independently represents hydrogen or (C₁₋₂)alkyl; or a 5-membered heteroaryl group containing one to four nitrogen atoms, wherein said 5-membered heteroaryl group is independently unsubstituted or mono-substituted with (C₁₋₄)alkyl; and R^{2B} represents hydrogen, or methyl; or R^{2A} and R^{2B} form, together with the carbon atom to which they are attached, a ring of 3- to 6 members, wherein the members needed to complete said ring are each independently selected from —CH₂— and —O— and wherein said ring does not contain more than one-O-member; or R^{2A} and R^{X3} together represent a —CH₂—O—* bridge, wherein the asterisk indicates the bond which is linked to the aromatic carbon atom, and R^{2B} represents hydrogen; R³ represents —SFs; (C₁₋₂)alkyi-S(O)₂-; or a fragment

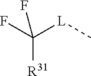

wherein R³¹ represents hydrogen, or fluoro; and L represents a direct bond, cycloprop-1,1-diyl, —CHR^{L}—O—*, —O—CH₂—*, —CH₂—NH—*, —CH₂—N(CH₃)—*, —O—, or —(SO₂)—; wherein R^{L} represents hydrogen, (C₁₋)alkyl, CH₃—O—CH₂—, or (CH₃)₂NCH₂—; wherein the asterisks indicate the bond which is linked to the aromatic carbon atom; or R³ and R^{X1} together represent a —O—CF₂—O— bridge; or R³ and R^{X2} together represent a —O—CF₂—O— bridge; and R⁴ represents hydrogen, halogen, or (C₁₋₄)alkyl; or a salt thereof.

In further embodiments, R¹ represents hydrogen; or a salt thereof (meaning a salt of compound of Formula 54).

In further embodiments, R^{Y1}, R^{Y2}, R^{Y3} and R^{Y4} all represent hydrogen; or a salt thereof.

In further embodiments, R^{2A} represents hydrogen, (C₁₋₄)alkyl, (C₃₋₆)cycloalkyl, (C₁₋₄)fluoroalkyl, (C₁₋₄)hydroxyalkyl, (C₁₋₄)alkoxy-(C₁₋₂)alkyl, (C₁₋₂)alkyl-S—(C₁₋₂)alkyl, (C₁₋₂)alkyl-(SO₂)—(C₁₋₂)alkyl, cyano, (C₁₋₂) cyanoalkyl, H₂N—C(O)—(C₁₋₂)alkyl, (R^{N1})₂N—(C₁₋₂)alkyl, or (R^{N1})₂N—C(O)—, wherein R^{N1} independently represents hydrogen or (C₁₋₂)alkyl; and R^{2B} represents hydrogen; or R^{2A} and R^{2B} form, together with the carbon atom to which they are attached, a ring of 3- to 6 members selected from cycloprop-1,1-diyl, cyclobut-1,1-diyl, oxetane-3,3-diyl and tetrahydropyran-4,4-diyl; or a salt thereof.

In further embodiments, R^{2A} represents hydrogen, methyl, or hydroxymethyl; and R^{2B} represents hydrogen; or a salt thereof.

In further embodiments, R³ represents a fragment

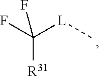

wherein R³¹ represents hydrogen, or fluoro; and L represents a direct bond, —CHR^{L}—O—*, —O—CH₂—*, —CH₂—NH—*, —CH₂—N(CH₃)—*, or —O—; wherein R^{L} represents hydrogen, methyl, CH₃—O—CH₂—, or (CH₃)₂NCH₂-; wherein the asterisks indicate the bond which is linked to the aromatic carbon atom; or a salt thereof.

In further embodiments, R⁴ represents hydrogen; or a salt thereof.

In further embodiments, the fragment

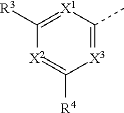

represents:

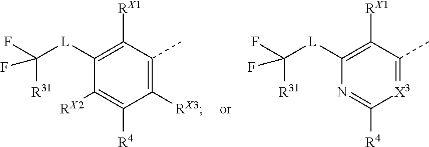

wherein X³ represents nitrogen and X¹ represents CR^{X1}; X¹ represents nitrogen and X³ represents CR^{X3}; or X¹ represents CR^{X1} and X³ represents CR^{X3}; or a salt thereof.

In further embodiments, the fragment

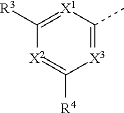

represents

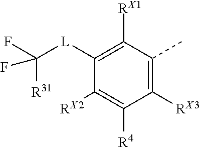

wherein R^{X1} represents hydrogen, fluoro, chloro, methyl, or methoxy; R^{X2} represents hydrogen; R^{X3} represents hydrogen, fluoro, or chloro; R⁴ represents hydrogen, fluoro, chloro, or methyl; R³¹ represents hydrogen, or fluoro; and L represents a direct bond, —CHR^{L}—O—*, —CH₂—NH—*, —CH₂—N(CH₃)—*, —O—, or; wherein R^{L} represents hydrogen, or methyl; wherein the asterisks indicate the bond which is linked to the aromatic carbon atom; or

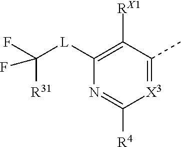

wherein X³ represents nitrogen and X¹ represents CR^{X1}; X¹ represents nitrogen and X³ represents CR^{X3}; or X¹ represents CR^{X1} and X³ represents CR^{X3}; wherein R^{X1} represents hydrogen, or methoxy; R^{X3} represents hydrogen; R⁴ represents hydrogen, or methyl; and L represents —CHR^{L}—O—*, —CH₂—NH—*, or —CH₂—N(CH₃)—*; wherein R^{L} represents hydrogen, methyl, CH₃—O—CH₂—, or (CH₃)₂NCH₂—; wherein the asterisks indicate the bond which is linked to the aromatic carbon atom; or a salt thereof.

In further embodiments, X¹ represents CR^{X1}; wherein R^{X1} represents hydrogen; X² represents nitrogen or CR^{X2}; wherein R^{X2} represents hydrogen; X³ represents nitrogen or CR^{X3}; wherein R^{X3} represents hydrogen; R¹ represents hydrogen; Y represents —CH₂—, or —CH₂—CH₂-; R^{2A} represents hydrogen, or hydroxymethyl; R^{2B} represents hydrogen; R³ represents trifluoromethyl or 2,2,2-trifluoroethoxy; and R⁴ represents hydrogen; or a salt thereof.

In further embodiments, the Kv7 Channel activator is selected from the group consisting of: 1-Spiro[3.3]hept-2-yl-3-(3-trifluoromethyl-benzyl)-urea; 1-Spiro[2.3]hex-5-yl-3-(3-trifluoromethyl-benzyl)-urea; 1-(1, 1-Difluoro-spiro[2.3]hex-5-yl)-3-(3-trifluoromethyl-benzyl)-urea; 1-((3r,5r)-1,1-Difluoro-spiro[2.3]hex-5-yl)-3-(3-trifluoromethyl-benzyl)-urea; 1-((3s,5s)-1,1-Difluoro-spiro[2.3]hex-5-yl)-3-(3-trifluoromethyl-benzyl)-urea; 1-(6,6-Difluoro-spiro[3.3]hept-2-yl)-3-(3-trifluoromethyl-benzyl)-urea; 1-[1-(3-Difluoromethoxy-phenyl)-cyclopropyl]-3-spiro[3.3]hept-2-yl-urea; 1-(2-Fluoro-3-trifluoromethoxy-benzyl)-3-spiro[3.3]hept-2-yl-urea; 1-[2-Methoxy-1-(3-trifluoromethyl-phenyl)-ethyl]-3-spiro[3.3]hept-2-yl-urea; 1-[1-(2-Difluoromethoxy-pyridin-4-yl)-2-methoxy-ethyl]-3-spiro[3.3]hept-2-yl-urea; 1-Spiro[3.3]hept-2-yl-3-(5-trifluoromethoxy-2,3-dihydro-benzofuran-3-yl)-urea; 1-(2-Fluoro-3-trifluoromethyl-benzyl)-3-spiro[3.3]hept-2-yl-urea; 1-(3-Difluoromethoxy-benzyl)-3-spiro[3.3]hept-2-yl-urea; 1-Spiro[3.3]hept-2-yl-3-[3-(2,2,2-trifluoro-ethoxy)-benzyl]-urea; 1-(3-Difluoromethyl-benzyl)-3-spiro[3.3]hept-2-yl-urea; 1-(2-Fluoro-5-trifluoromethyl-benzyl)-3-spiro[3.3]hept-2-yl-urea; 1-(4-Fluoro-3-trifluoromethyl-benzyl)-3-spiro[3.3]hept-2-yl-urea; 1-(3-Fluoro-5-trifluoromethyl-benzyl)-3-spiro[3.3]hept-2-yl-urea; 1-[1-(2-Bromo-5-trifluoromethyl-phenyl)-ethyl]-3-spiro[3.3]hept-2-yl-urea; 1-[1-(2-Methoxy-5-trifluoromethyl-phenyl)-ethyl]-3-spiro[3.3]hept-2-yl-urea; 1-[1-(2-Methoxy-3-trifluoromethyl-phenyl)-ethyl]-3-spiro[3.3]hept-2-yl-urea; 1-(2-Methoxy-3-trifluoromethyl-benzyl)-3-spiro[3.3]hept-2-yl-urea; 1-Spiro[3.3]hept-2-yl-3-[1-(3-trifluoromethyl-phenyl)-ethyl]-urea; 1-[Cyclopropyl-(3-trifluoromethyl-phenyl)-methyl]-3-spiro[3.3]hept-2-yl-urea; 1-[Cyclopropyl-(3-difluoromethoxy-phenyl)-methyl]-3-spiro[3.3]hept-2-yl-urea; 1-{1-[4-Methyl-3-(2,2,2-trifluoro-ethoxy)-phenyl]-ethyl}-3-spiro[3.3]hept-2-yl-urea; 1-{1-[4-Chloro-3-(2,2,2-trifluoro-ethoxy)-phenyl]-ethyl}-3-spiro[3.3]hept-2-yl-urea; 1-{1-[4-Methoxy-3-(2,2,2-trifluoro-ethoxy)-phenyl]-ethyl}-3-spiro[3.3]hept-2-yl-urea; 1-{1-[2-Methyl-3-(2,2,2-trifluoro-ethoxy)-phenyl]-ethyl}-3-spiro[3.3]hept-2-yl-urea; 1-{1-[2-Chloro-3-(2,2,2-trifluoro-ethoxy)-phenyl]-ethyl}-3-spiro[3.3]hept-2-yl-urea; 1-{1-[2-Methoxy-3-(2,2,2-trifluoro-ethoxy)-phenyl]-ethyl}-3-spiro[3.3]hept-2-yl-urea; 1-{1-[3-Methyl-5-(2,2,2-trifluoro-ethoxy)-phenyl]-ethyl}-3-spiro[3.3]hept-2-yl-urea; 1-{1-[3-Chloro-5-(2,2,2-trifluoro-ethoxy)-phenyl]-ethyl}-3-spiro[3.3]hept-2-yl-urea; 1-(1-(3-(pentafluoro-16-sulfaneyl)phenyl)ethyl)-3-(spiro[3.3]heptan-2-yl) urea; 1-(3-(pentafluoro-16-sulfaneyl)benzyl)-3-(spiro[3.3]heptan-2-yl) urea; 1-[1-(3-Methanesulfonyl-phenyl)-ethyl]-3-spiro[3.3]hept-2-yl-urea; 1-Spiro[3.3]hept-2-yl-3-[3-(1-trifluoromethyl-cyclopropyl)-benzyl]-urea; 1-(2-Hydroxy-5-trifluoromethyl-benzyl)-3-spiro[3.3]hept-2-yl-urea; 1-{1-[2-Chloro-5-(2,2,2-trifluoro-ethoxy)-phenyl]-ethyl}-3-spiro[3.3]hept-2-yl-urea; 1-{1-[2-Methoxy-5-(2,2,2-trifluoro-ethoxy)-phenyl]-ethyl}-3-spiro[3.3]hept-2-yl-urea; 1-Spiro[3.3]hept-2-yl-3-(3-trifluoromethoxy-benzyl)-urea; 1-Spiro[3.3]hept-2-yl-3-(3-trifluoromethanesulfonyl-benzyl)-urea; 1-Spiro[3.3]hept-2-yl-3-(3-trifluoromethoxymethyl-benzyl)-urea; 1-Spiro[3.3]hept-2-yl-3-[3-(3-trifluoromethyl-phenyl)-oxetan-3-yl]-urea; 1-[2-Hydroxy-2-methyl-1-(3-trifluoromethyl-phenyl)-propyl]-3-spiro[3.3]hept-2-yl-urea; 1-(2,2-Difluoro-benzo[1,3]dioxol-4-ylmethyl)-3-spiro[3.3]hept-2-yl-urea; 1-[1-(2,2-Difluoro-benzo[1,3]dioxol-4-yl)-ethyl]-3-spiro[3.3]hept-2-yl-urea; 1-{2-Dimethylamino-1-[6-(2,2,2-trifluoro-ethoxy)-pyridin-2-yl]-ethyl}-3-spiro[3.3]hept-2-yl-urea; 1-Spiro[3.3]hept-2-yl-3-{4-[2-(2,2,2-trifluoro-ethoxy)-pyridin-4-yl]-tetrahydro-pyran-4-yl}-urea; 1-{2-Methoxy-1-[2-(2,2,2-trifluoro-ethoxy)-pyridin-4-yl]-ethyl}-1-methyl-3-spiro[3.3]hept-2-yl-urea; 1-[1-(2-Difluoromethoxy-pyridin-4-yl)-cyclobutyl]-3-spiro[3.3]hept-2-yl-urea; 1-[1-(2-Difluoromethoxy-pyridin-4-yl)-1-methyl-ethyl]-3-spiro[3.3]hept-2-yl-urea; 5-(2,2,2-Trifluoro-ethoxy)-3,4-dihydro-1H-[2,6]naphthyridine-2-carboxylic acid spiro[3.3]hept-2-ylamide; 1-Methoxymethyl-5-(2,2,2-trifluoro-ethoxy)-3,4-dihydro-1H-isoquinoline-2-carboxylic acid spiro[3.3]hept-2-ylamide; 1-[Cyano-(3-trifluoromethyl-phenyl)-methyl]-3-spiro[3.3]hept-2-yl-urea; 1-Spiro[3.3]hept-2-yl-3-[(1H-[1,2,4]triazol-3-yl)-(3-trifluoromethyl-phenyl)-methyl]-urea; 1-[2,2-Difluoro-1-(3-trifluoromethyl-phenyl)-ethyl]-3-spiro[3.3]hept-2-yl-urea; 1-Spiro[3.3]hept-2-yl-3-[2,2,2-trifluoro-1-(3-trifluoromethyl-phenyl)-ethyl]-urea; 1-Spiro[3.3]hept-2-yl-3-[6-(2,2,2-trifluoro-ethoxy)-pyrimidin-4-ylmethyl]-urea; 1-[2-Methyl-6-(2,2,2-trifluoro-ethoxy)-pyrimidin-4-ylmethyl]-3-spiro[3.3]hept-2-yl-urea; 1-Spiro[3.3]hept-2-yl-3-{1-[6-(2,2,2-trifluoro-ethoxy)-pyrimidin-4-yl]-ethyl}-urea; 2-(3-Spiro[3.3]hept-2-yl-ureido)-2-(3-trifluoromethyl-phenyl)-acetamide; N-Methyl-2-(3-spiro[3.3]hept-2-yl-ureido)-2-(3-trifluoromethyl-phenyl)-acetamide; 1-[2-Methanesulfonyl-1-(3-trifluoromethoxy-phenyl)-ethyl]-3-spiro[3.3]hept-2-yl-urea; 1-[1-(2,2-Difluoro-benzo[1,3]dioxol-5-yl)-ethyl]-3-spiro[3.3]hept-2-yl-urea; 1-[2-Cyano-1-(3-trifluoromethoxy-phenyl)-ethyl]-3-spiro[3.3]hept-2-yl-urea; 1-[2-Cyano-1-(3-trifluoromethyl-phenyl)-ethyl]-3-spiro[3.3]hept-2-yl-urea; 1-{1-[2-Methyl-6-(2,2,2-trifluoro-ethoxy)-pyrimidin-4-yl]-ethyl}-3-spiro[3.3]hept-2-yl-urea; 1-[2-Methanesulfonyl-1-(3-trifluoromethyl-phenyl)-ethyl]-3-spiro[3.3]hept-2-yl-urea; 1-Spiro[3.3]hept-2-yl-3-[2-(2,2,2-trifluoro-ethoxy)-pyrimidin-4-ylmethyl]-urea; 1-[2-(2-Methoxy-ethoxy)-1-(3-trifluoromethyl-phenyl)-ethyl]-3-spiro[3.3]hept-2-yl-urea; 1-[2-Isopropoxy-1-(3-trifluoromethyl-phenyl)-ethyl]-3-spiro[3.3]hept-2-yl-urea; 1-[3-Ethoxy-1-(3-trifluoromethyl-phenyl)-propyl]-3-spiro[3.3]hept-2-yl-urea; 1-[3-Hydroxy-1-(3-trifluoromethyl-phenyl)-propyl]-3-spiro[3.3]hept-2-yl-urea; 1-[3-Methylsulfanyl-1-(3-trifluoromethyl-phenyl)-propyl]-3-spiro[3.3]hept-2-yl-urea; 1-[2-Hydroxy-1-(3-trifluoromethyl-phenyl)-ethyl]-3-spiro[3.3]hept-2-yl-urea; 1-{3-Ethoxy-1-[2-(2,2,2-trifluoro-ethoxy)-pyridin-4-yl]-propyl)-3-spiro[3.3]hept-2-yl-urea; 1-{2-Methoxy-1-[6-(2,2,2-trifluoro-ethoxy)-pyrimidin-4-yl]-ethyl}-3-spiro[3.3]hept-2-yl-urea; 4-(3-Spiro[3.3]hept-2-yl-ureido)-4-(3-trifluoromethyl-phenyl)-butyramide; 1-Spiro[3.3]hept-2-yl-3-[1-(3-trifluoromethyl-phenyl)-but-3-enyl]-urea; 1-[3-Methanesulfonyl-1-(3-trifluoromethyl-phenyl)-propyl]-3-spiro[3.3]hept-2-yl-urea; 1-{2-Hydroxy-1-[2-methyl-6-(2,2,2-trifluoro-ethoxy)-pyrimidin-4-yl]-ethyl}-3-spiro[3.3]hept-2-yl-urea; 1-{2-Hydroxy-1-[6-(2,2,2-trifluoro-ethoxy)-pyrimidin-4-yl]-ethyl}-3-spiro[3.3]hept-2-yl-urea; 1-[3,3-Difluoro-1-(3-trifluoromethyl-phenyl)-propyl]-3-spiro[3.3]hept-2-yl-urea; 1-{3-Hydroxy-1-[6-(2,2,2-trifluoro-ethoxy)-pyrimidin-4-yl]-propyl}-3-spiro[3.3]hept-2-yl-urea; 1-[1-(3-Methanesulfonyl-phenyl)-2-methoxy-ethyl]-3-spiro[3.3]hept-2-yl-urea; 1-{2-Hydroxy-1-[3-(2,2,2-trifluoro-ethoxy)-phenyl]-ethyl}-3-spiro[3.3]hept-2-yl-urea; 1-{3-Hydroxy-1-[3-(2,2,2-trifluoro-ethoxy)-phenyl]-propyl)-3-spiro[3.3]hept-2-yl-urea; 1-[Cyano-(3-trifluoromethyl-phenyl)-methyl]-3-spiro[3.3]hept-2-yl-urea; 1-Spiro[3.3]hept-2-yl-3-{1-[3-(2,2,2-trifluoro-ethoxy)-phenyl]-ethyl}-urea; 1-{2-Methoxy-1-[3-(2,2,2-trifluoro-ethoxy)-phenyl]-ethyl}-3-spiro[3.3]hept-2-yl-urea; 1-{2-Methoxy-1-[2-(2,2,2-trifluoro-ethoxy)-pyridin-4-yl]-ethyl}-3-spiro[3.3]hept-2-yl-urea; 1-Spiro[3.3]hept-2-yl-3-{1-[2-(2,2,2-trifluoro-ethoxy)-pyridin-4-yl]-ethyl}-urea; 1-{2-Dimethylamino-1-[2-(2,2,2-trifluoro-ethoxy)-pyridin-4-yl]-ethyl}-3-spiro[3.3]hept-2-yl-urea; 1-{1-[3-Fluoro-5-(2,2,2-trifluoro-ethoxy)-phenyl]-ethyl}-3-spiro[3.3]hept-2-yl-urea; 1-Spiro[3.3]hept-2-yl-3-(2-trifluoromethoxy-pyridin-4-ylmethyl)-urea; 1-Spiro[3.3]hept-2-yl-3-[1-(2-trifluoromethoxy-pyridin-4-yl)-ethyl]-urea; 1-{1-[2-Fluoro-3-(2,2,2-trifluoro-ethoxy)-phenyl]-ethyl}-3-spiro[3.3]hept-2-yl-urea; 1-{1-[2-Huoro-5-(2,2,2-trifluoro-ethoxy)-phenyl]-ethyl}-3-spiro[3.3]hept-2-yl-urea; 1-[1-(2-Difluoromethoxy-pyridin-4-yl)-ethyl]-3-spiro[3.3]hept-2-yl-urea; 1-(Spiro[3.3]heptan-2-yl)-3-(1-(2-((1,1,1-trifluoropropan-2-yl)oxy)pyridin-4-yl)ethyl) urea; 1-(Spiro[3.3]heptan-2-yl)-3-(1-(3-((1,1,1-trifluoropropan-2-yl)oxy)phenyl)ethyl) urea; 1-Spiro[3.3]hept-2-yl-3-[2-(2,2,2-trifluoro-ethoxy)-pyridin-4-ylmethyl]-urea; 1-Spiro[3.3]hept-2-yl-3-[6-(2,2,2-trifluoro-1-methoxymethyl-ethoxy)-pyridin-2-ylmethyl]-urea; 1-[6-(1-Dimethylaminomethyl-2,2,2-trifluoro-ethoxy)-pyridin-2-ylmethyl]-3-spiro[3.3]hept-2-yl-urea; 1-[2-(1-Dimethylaminomethyl-2,2,2-trifluoro-ethoxy)-pyridin-4-ylmethyl]-3-spiro[3.3]hept-2-yl-urea; 1-Spiro[3.3]hept-2-yl-3-[2-(2,2,2-trifluoro-1-methoxymethyl-ethoxy)-pyridin-4-ylmethyl]-urea; 1-[5-Methyl-6-(2,2,2-trifluoro-ethoxy)-pyridin-2-ylmethyl]-3-spiro[3.3]hept-2-yl-urea; 1-[4-Methyl-3-(2,2,2-trifluoro-ethoxy)-benzyl]-3-spiro[3.3]hept-2-yl-urea; 1-[3-Huoro-5-(2,2,2-trifluoro-1-methyl-ethoxy)-benzyl]-3-spiro[3.3]hept-2-yl-urea; 1-Spiro[3.3]hept-2-yl-3-{1-[3-(2,2,2-trifluoro-1-methyl-ethoxy)-phenyl]-cyclopropyl}-urea; 1-Spiro[3.3]hept-2-yl-3-[4-(2,2,2-trifluoro-ethoxy)-pyrimidin-2-ylmethyl]-urea; 1-{1-[4-Huoro-3-(2,2,2-trifluoro-ethoxy)-phenyl]-ethyl}-3-spiro[3.3]hept-2-yl-urea; 1-[4-Huoro-3-(2,2,2-trifluoro-ethoxy)-benzyl]-3-spiro[3.3]hept-2-yl-urea; 1-{2-[Methyl-(2,2,2-trifluoro-ethyl)-amino]-pyridin-4-ylmethyl}-3-spiro[3.3]hept-2-yl-urea; 1-Spiro[3.3]hept-2-yl-3-[2-(2,2,2-trifluoro-ethylamino)-pyridin-4-ylmethyl]-urea; 1-Spiro[3.3]hept-2-yl-3-[4-(2,2,2-trifluoro-1-methyl-ethoxy)-pyridin-2-ylmethyl]-urea; 1-Spiro[3.3]hept-2-yl-3-[2-(2,2,2-trifluoro-1-methyl-ethoxy)-pyrimidin-4-ylmethyl]-urea; 1-Spiro[3.3]hept-2-yl-3-[1-(3-trifluoromethoxy-phenyl)-ethyl]-urea; 1-Spiro[2.3]hex-5-yl-3-[6-(2,2,2-trifluoro-1-methyl-ethoxy)-pyrimidin-4-ylmethyl]-urea; 1-[1-(2,2-Difluoro-benzo[1,3]dioxol-4-yl)-ethyl]-3-spiro[2.3]hex-5-yl-urea; 1-{2-Hydroxy-1-[6-(2,2,2-trifluoro-ethoxy)-pyrimidin-4-yl]-ethyl}-3-spiro[2.3]hex-5-yl-urea; 1-{2-Hydroxy-1-[2-methyl-6-(2,2,2-trifluoro-ethoxy)-pyrimidin-4-yl]-ethyl}-3-spiro[2.3]hex-5-yl-urea; 1-[1-(3-Difluoromethoxy-phenyl)-ethyl]-3-spiro[2.3]hex-5-yl-urea; 1-(2-Methoxy-5-trifluoromethyl-benzyl)-3-spiro[2.3]hex-5-yl-urea; 1-Spiro[2.3]hex-5-yl-3-[4-(2,2,2-trifluoro-ethoxy)-pyrimidin-2-ylmethyl]-urea; 1-(1-(3-(pentafluoro-16-sulfaneyl)phenyl)ethyl)-3-(spiro[2.3]hexan-5-yl) urea; 1-(2-Methoxy-3-trifluoromethyl-benzyl)-3-spiro[2.3]hex-5-yl-urea; 1-{1-[2-Huoro-5-(2,2,2-trifluoro-ethoxy)-phenyl]-ethyl}-3-spiro[2.3]hex-5-yl-urea; 1-(3-(pentafluoro-16-sulfaneyl)benzyl)-3-(spiro[2.3]hexan-5-yl) urea; 1-{1-[2-Huoro-3-(2,2,2-trifluoro-ethoxy)-phenyl]-ethyl}-3-spiro[2.3]hex-5-yl-urea; 1-{1-[3-Huoro-5-(2,2,2-trifluoro-ethoxy)-phenyl]-ethyl}-3-spiro[2.3]hex-5-yl-urea; 1-[1-(2-Methoxy-3-trifluoromethyl-phenyl)-ethyl]-3-spiro[2.3]hex-5-yl-urea; 1-(2-Huoro-3-trifluoromethoxy-benzyl)-3-spiro[2.3]hex-5-yl-urea; 1-Spiro[2.3]hex-5-yl-3-{1-[3-(2,2,2-trifluoro-1-methyl-ethoxy)-phenyl]-cyclopropyl}-urea; 1-[1-(3-Difluoromethoxy-phenyl)-cyclopropyl]-3-spiro[2.3]hex-5-yl-urea; 1-[1-(2-Methoxy-5-trifluoromethyl-phenyl)-ethyl]-3-spiro[2.3]hex-5-yl-urea; 1-[3-Hydroxy-1-(3-trifluoromethoxy-phenyl)-propyl]-3-spiro[2.3]hex-5-yl-urea; 1-[1-(2-Difluoromethoxy-pyridin-4-yl)-ethyl]-3-spiro[2.3]hex-5-yl-urea; 1-Spiro[2.3]hex-5-yl-3-[1-(3-trifluoromethyl-phenyl)-ethyl]-urea; 1-Spiro[2.3]hex-5-yl-3-[1-(3-trifluoromethoxy-phenyl)-ethyl]-urea; 1-(3-Huoro-5-trifluoromethyl-benzyl)-3-spiro[2.3]hex-5-yl-urea; 1-[2-Hydroxy-1-(3-trifluoromethyl-phenyl)-ethyl]-3-spiro[2.3]hex-5-yl-urea; 1-Spiro[2.3]hex-5-yl-3-{1-[6-(2,2,2-trifluoro-ethoxy)-pyrimidin-4-yl]-ethyl}-urea; 1-{1-[2-Methyl-6-(2,2,2-trifluoro-ethoxy)-pyrimidin-4-yl]-ethyl}-3-spiro[2.3]hex-5-yl-urea; 1-[2,2-Difluoro-1-(3-trifluoromethyl-phenyl)-ethyl]-3-spiro[2.3]hex-5-yl-urea; 1-[2-Methoxy-1-(3-trifluoromethyl-phenyl)-ethyl]-3-spiro[2.3]hex-5-yl-urea; 1-(4-Huoro-3-trifluoromethyl-benzyl)-3-spiro[2.3]hex-5-yl-urea; 1-(2-Huoro-3-trifluoromethyl-benzyl)-3-spiro[2.3]hex-5-yl-urea; 1-(2-Chloro-3-trifluoromethyl-benzyl)-3-spiro[2.3]hex-5-yl-urea; 1-(2-Huoro-5-trifluoromethyl-benzyl)-3-spiro[2.3]hex-5-yl-urea; 1-{1-[5-Methoxy-6-(2,2,2-trifluoro-ethoxy)-pyrimidin-4-yl]-ethyl}-3-spiro[2.3]hex-5-yl-urea; 1-Spiro[2.3]hex-5-yl-3-(2-trifluoromethoxy-pyridin-4-ylmethyl)-urea; 1-{2-Methoxy-1-[2-(2,2,2-trifluoro-ethoxy)-pyridin-4-yl]-ethyl}-3-spiro[2.3]hex-5-yl-urea; 1-(3-Difluoromethoxy-benzyl)-3-spiro[2.3]hex-5-yl-urea; 1-Spiro[2.3]h ex-5-yl-3-(3-trifluoromethoxy-benzyl)-urea; 1-Spiro[2.3]hex-5-yl-3-[3-(2,2,2-trifluoro-ethoxy)-benzyl]-urea; 1-(3-Difluoromethyl-benzyl)-3-spiro[2.3]hex-5-yl-urea; 1-Spiro[2.3]hex-5-yl-3-[2-(2,2,2-trifluoro-ethoxy)-pyridin-4-ylmethyl]-urea; 1-(6,6-Difluoro-spiro[3.3]hept-2-yl)-3-[2-(2,2,2-trifluoro-ethoxy)-pyridin-4-ylmethyl]-urea; 1-(6,6-Difluoro-spiro[3.3]hept-2-yl)-3-[1-(3-trifluoromethyl-phenyl)-ethyl]-urea; 1-Spiro[3.3]hept-2-yl-3-[(1H-[1,2,3]triazol-4-yl)-(3-trifluoromethyl-phenyl)-methyl]-urea; 1-[(1-Methyl-1H-tetrazol-5-yl)-(3-trifluoromethyl-phenyl)-methyl]-3-spiro[3.3]hept-2-yl-urea; 1-[(2-Methyl-2H-tetrazol-5-yl)-(3-trifluoromethyl-phenyl)-methyl]-3-spiro[3.3]hept-2-yl-urea; 1-[(3-Methyl-3H-[1,2,3]triazol-4-yl)-(3-trifluoromethyl-phenyl)-methyl]-3-spiro[3.3]hept-2-yl-urea; 1-[1-(2-Difluoromethoxy-pyridin-4-yl)-ethyl]-3-(6-iodo-spiro[3.3]hept-2-yl)-urea; 1-[(R)-1-(2-Difluoromethoxy-pyridin-4-yl)-ethyl]-3-spiro[3.3]hept-2-yl-urea; 1-[(S)-1-(2-Difluoromethoxy-pyridin-4-yl)-ethyl]-3-spiro[3.3]hept-2-yl-urea; 1-Spiro[3.3]hept-2-yl-3-{1-[3-((R)-2,2,2-trifluoro-1-methyl-ethoxy)-phenyl]-cyclopropyl}-urea; 1-Spiro[3.3]hept-2-yl-3-{1-[3-((S)-2,2,2-trifluoro-1-methyl-ethoxy)-phenyl]-cyclopropyl}-urea; 1-Spiro[3.3]hept-2-yl-3-[2-((R)-2,2,2-trifluoro-1-methoxymethyl-ethoxy)-pyridin-4-ylmethyl]-urea; 1-Spiro[3.3]hept-2-yl-3-[2-((S)-2,2,2-trifluoro-1-methoxymethyl-ethoxy)-pyridin-4-ylmethyl]-urea; 1-[(S)-1-(3-Difluoromethoxy-phenyl)-ethyl]-3-spiro[3.3]hept-2-yl-urea; 1-[(R)-1-(3-Difluoromethoxy-phenyl)-ethyl]-3-spiro[3.3]hept-2-yl-urea; 1-Spiro[3.3]hept-2-yl-3-[(S)-1-(3-trifluoromethyl-phenyl)-ethyl]-urea; 1-Spiro[3.3]hept-2-yl-3-{(S)-1-[3-(2,2,2-trifluoro-ethoxy)-phenyl]-ethyl}-urea; 1-{(S)-1-[4-Fluoro-3-(2,2,2-trifluoro-ethoxy)-phenyl]-ethyl}-3-spiro[3.3]hept-2-yl-urea; 1-Spiro[3.3]hept-2-yl-3-[(R)-2,2,2-trifluoro-1-(3-trifluoromethyl-phenyl)-ethyl]-urea; 1-Spiro[3.3]hept-2-yl-3-[(R)-(1H-[1,2,4]triazol-3-yl)-(3-trifluoromethyl-phenyl)-methyl]-urea; 1-Spiro[3.3]hept-2-yl-3-[2-((R)-2,2,2-trifluoro-1-methyl-ethoxy)-pyrimidin-4-ylmethyl]-urea; 1-Spiro[3.3]hept-2-yl-3-[2-((S)-2,2,2-trifluoro-1-methyl-ethoxy)-pyrimidin-4-ylmethyl]-urea; 1-Spiro[3.3]hept-2-yl-3-{(S)-1-[6-(2,2,2-trifluoro-ethoxy)-pyrimidin-4-yl]-ethyl}-urea; 1-Spiro[3.3]hept-2-yl-3-{(R)-1-[6-(2,2,2-trifluoro-ethoxy)-pyrimidin-4-yl]-ethyl}-urea; 1-[(R)-2-Hydroxy-1-(3-trifluoromethyl-phenyl)-ethyl]-3-spiro[3.3]hept-2-yl-urea; 1-[(S)-2-Hydroxy-1-(3-trifluoromethyl-phenyl)-ethyl]-3-spiro[3.3]hept-2-yl-urea; 1-[(R)-2-Hydroxy-1-(3-trifluoromethyl-phenyl)-ethyl]-3-spiro[2.3]hex-5-yl-urea; 1-[(R)-1-(2-Difluoromethoxy-pyridin-4-yl)-2-methoxy-ethyl]-3-spiro[3.3]hept-2-yl-urea; 1-{(R)-2-Methoxy-1-[2-(2,2,2-trifluoro-ethoxy)-pyridin-4-yl]-ethyl}-3-spiro[2.3]hex-5-yl-urea; 1-[(S)-1-(2-Difluoromethoxy-pyridin-4-yl)-ethyl]-3-spiro[2.3]hex-5-yl-urea; or 1-[(S)-1-(2-Bromo-5-trifluoromethyl-phenyl)-ethyl]-3-spiro[3.3]hept-2-yl-urea; or a salt thereof.

### Formula 55

In another embodiment, the Kv7 channel activator may be selected from one of the following compounds of Formula 55. Such compounds are described in International Publication No. WO2020157126A1, published Aug. 6, 2020, and corresponding to International Application No. PCT/EP2020/052156 filed Jan. 29, 2020; which is incorporated by reference in its entirety herein. In the case of any conflict of terminology in the context of Formula 55, this reference incorporated by reference herein controls.

In an embodiment, the Kv7 channel activator is a compound according to Formula 55:

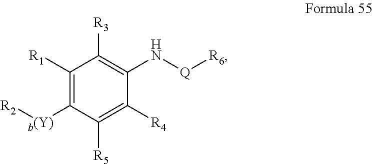

or a tautomer, salt, solvate, stereoisomer or isotope thereof, wherein: Y is selected from: C1-C10 alkylamino, C1-C10 alkyl, C1-C10 alkoxy, C3-C8 cycloalkyl, C1-C10 alkylcarbonyl, Cl-CIO alkylaminocarbonyl, Cl-CIO alkylthio, aryl, heterocycle, Cl-CIO carbonyl, C1-C10 amide and C1-C10 carboxyl and it is optionally mono-substituted or bi-substituted with a first substituent independently selected from: hydroxyl, amino, thiol, carboxylic acid, amide, carbonyl, halogen, C1-C6 alkyl, C3-C6 cycloalkyl, C1-C6 alkylcarbonyl, C1-C6 alkoxy carbonyl, C1-C6 alkylamino, C1-C6 alkylaminocarbonyl, C1-C6 alkyloxy, C1-C6 alkylthio, C2-C8 alkenyl, C5-C7 cycloalkenyl, C2-C8 alkynyl and C5-C10 aryl, any of said first substituents being optionally substituted by one or more second substituents independently selected from: hydroxyl, amino, thiol, carboxylic acid, amide, carbonyl, halogen, C1-C6 alkyl, C3-C6 cycloalkyl, C1-C6 alkylcarbonyl, C1-C6 alkoxy carbonyl, C1-C6 alkylamino, C1-C6 alkylaminocarbonyl, C1-C6 alkyloxy, C1-C6 alkylthio, C2-C8 alkenyl, C5-C7 cycloalkenyl, C2-C8 alkynyl and C5-C10 aryl; b is an integer comprised between 1 and 3; or Y is NH or O and b is 1; R.2 is selected from: an aromatic ring, a cycloalkyl, a heterocycle, a linear or branched saturated alkyl and a linear or branched unsaturated alkyl, any of which optionally bearing in one or more positions at least one substituent independently selected from the group consisting of: OH, NO2, CN, halogen, NH2, C1-C3 haloalkyl, C1-C3 haloalkoxy, C1-C6 alkyl, C3-C6 cycloalkyl, C1-C6 alkylcarbonyl, C1-C6 alkoxy carbonyl, C1-C6 alkylamino, C1-C6 dialkylamino, C1-C6 alkylaminocarbonyl, acetamidyl, C1-C6 alkyloxy, C1-C6 alkylthio, C2-C8 alkenyl, C5-C7 cycloalkenyl, C2-C8 alkynyl, C5-C10 aryl, thiol and thiol ether; Ri, R3, R4 and R5 are each independently selected from the group consisting of: H, N3/4, OH, halogen, C1-C6 alkyl, C3-C6 cycloalkyl, C1-C6 alkylcarbonyl, C1-C6 alkoxy carbonyl, C1-C6 alkylamino, C2-C6 dialkylamino, C1-C6 alkylaminocarbonyl, C1-C6 alkyloxy, C1-C6 alkylthio, C2-C8 alkenyl, C5-C7 cycloalkenyl, heterocycle, C2-C8 alkynyl and C5-C10 aryl, any of which being optionally substituted by one or more substituents independently selected from: hydroxyl, amine, thiol, carboxyl, carboxylic acid, and halogen; Q is selected from the group consisting of: C=0, SO2, SO, SO2NH, CONH, CSNH, C≡NH, CNHNH, and C═S; when Q is C=0 or C═S, R₆ is selected from: linear or branched C3-C30 alkyl, linear or branched C5-C30 arylalkyl, C3-C8 cycloalkyl, linear or branched C2-C4 alkyl substituted by C3-C8 cycloalkyl, linear or branched C3-C10 cycloalkylamino, linear or branched Cl-CIO alkylamino, linear or branched C2-C10 alkenylamino, linear or branched C2-C10 alkynylamino, C5-C10 aryl, linear or branched C7-C15 arylalkylamino, linear or branched C7-C15 arylalkenylamino, linear or branched C7-C15 arylalkynylamino, C2-C30 alkenyl, C2-C30 alkynyl and C1-C30 alkoxyl, any of which being optionally substituted with one or more substituents independently selected from: linear or branched alkyl group, halogen, hydroxyl group, amine group, alkylamine, dialkylamine, thiol, thioether and cycloalkyl; when Q is SO2, SO, SO2NH, CONH, CSNH, C≡NH, or CNHNH, R₆ is selected from: linear or branched C1-C30 alkyl, C3-C8 cycloalkyl, linear or branched C1-C30 alkylcarbonyl, linear or branched C1-C30 alkoxycarbonyl, linear or branched C1-C30 alkylamino, linear or branched C1-C30 alkylaminocarbonyl, linear or branched C1-C30 alkyloxy, linear or branched C1-C30 alkylthio, linear or branched C2-C30 alkenyl, C5-C8 cycloalkenyl, linear or branched C2-C30 alkynyl, heterocycle and aryl, any of which being optionally substituted with one or more substituents independently selected from: halogen, C1-C6 alkyl, oxo and C3-C7 cycloalkyl.

In further embodiments, b is 1.

In further embodiments, Q is C=0 or SO2.

In further embodiments, Y is Cl-CIO alkylamino, Cl-CIO alkyl or CI-CIO alkoxy.

In further embodiments, R2 is an aromatic ring optionally substituted with one or more substituents independently selected from the group consisting of: OH, NO2, halogen, NH2, C1-C3 haloalkyl, acetamidyl, C1-C6 alkyloxy, C1-C3 haloalkoxy, and C1-C6 alkylcarbonyl.

In further embodiments, Ri, R3, R4 and R5 are each independently: H, C1-C6 alkyl, C3-C6 cycloalkyl, NH2, NH(C1-C6 alkyl), N(C1-C6 alkyl)2, azepane, pirrolidine, piperidine, aziridine or halogen.

In further embodiments, when Q is C=0 or C═S, R₆ is selected from: linear or branched C3-C30 alkyl, linear or branched C5-C30 arylalkyl, C3-C8 cycloalkyl, linear or branched C2-C4 alkyl substituted by C3-C8 cycloalkyl, linear or branched C1-C10 alkylamino, linear or branched C7-C15 arylalkylamino, C2-C30 alkenyl, C2-C30 alkynyl and C1-C30 alkoxyl, any of which being optionally substituted with one or more substituents independently selected from: halogen and cycloalkyl; and where Q is S0₂, SO, SO2NH, CONH, CSNH, C≡NH, or CNHNH, Re is C3-C8 cycloalkyl or aryl and is optionally substituted with one or more C1-C6 alkyl.

In further embodiments, the Kv7 Channel activator is selected from the group consisting of those listed in the following table

| COMPOUND STRUCTURE | COMPOUND NAME |
|---|---|
| 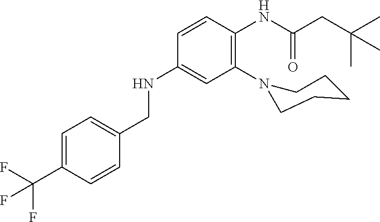 [SMILES: CC(C)(C)CC(=O)Nc1ccc(NCc2ccc(C(F)(F)F)cc2)cc1N1CCCCC1] | N-(2-(piperidin-1-yl)-4-((4- (trifluoromethyl)benzyl)amino) phenyl)heptanamide |
| 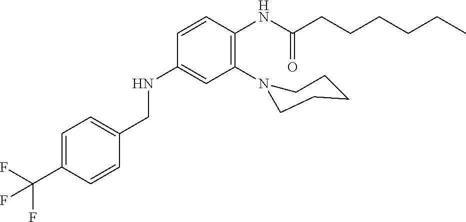 [SMILES: CCCCCCC(=O)Nc1ccc(NCc2ccc(C(F)(F)F)cc2)cc1N1CCCCC1] | N-(2-(piperidin-1-yl)-4-((4- (trifluoromethyl)benzyl)amino) phenyl)heptanamide |
| 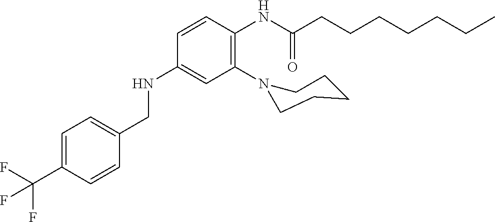 [SMILES: CCCCCCCC(=O)Nc1ccc(NCc2ccc(C(F)(F)F)cc2)cc1N1CCCCC1] | N-(2-(piperidin-1-yl)-4-((4- (trifluoromethyl)benzyl)amino) phenyl)octanamide |
| 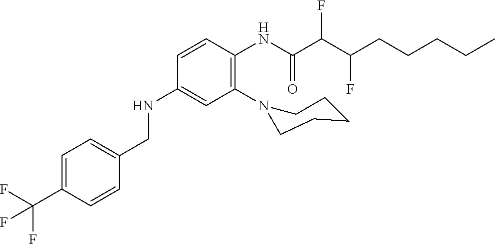 [SMILES: CCCCCC(F)C(F)C(=O)Nc1ccc(NCc2ccc(C(F)(F)F)cc2)cc1N1CCCCC1] | 2,3-difluoro-N-(2-(piperidin-1- yl)-4-((4- (trifluoromethyl)benzyl)amino) phenyl)octanamide (2R,3S)-2,3-difluoro-N-(2- (piperidin-1-yl)-4-((4- (trifluoromethyl)benzyl)amino) phenyl)octanamide (2S,3R)-2,3-difluoro-N-(2- (piperidin-1-yl)-4-((4- (trifluoromethyl)benzyl)amino) phenyl)octanamide (2R,3R)-2,3-difluoro-N-(2- (piperidin-1-yl)-4-((4- (trifluoromethyl)benzyl)amino) |
| | phenyl)octanamide |
| | (2S,3S)-2,3-difluoro-N-(2- |
| | (piperidin-1-yl)-4-((4- |
| | (trifluoromethyl)benzyl)amino) |
| | phenyl)octanamide |
| 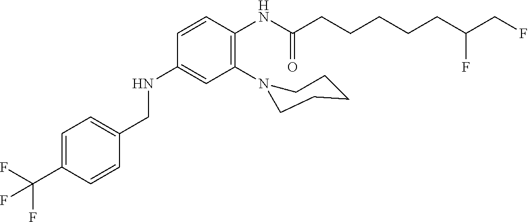 [SMILES: O=C(CCCCCC(F)CF)Nc1ccc(NCc2ccc(C(F)(F)F)cc2)cc1N1CCCCC1] | 7,8-difluoro-N-(2-(piperidin-1- yl)-4-((4- (trifluoromethyl)benzyl)amino) phenyl)octanamide (7R)-7,8-difluoro-N-(2- (piperidin-1-yl)-4-((4- (trifluoromethyl)benzyl)amino) phenyl)octanamide (7S)-7,8-difluoro-N-(2- (piperidin-1-yl)-4-((4- (trifluoromethyl)benzyl)amino) phenyl)octanamide |
| 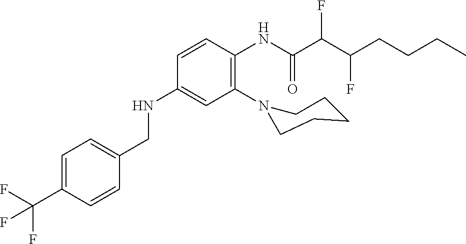 [SMILES: CCCCC(F)C(F)C(=O)Nc1ccc(NCc2ccc(C(F)(F)F)cc2)cc1N1CCCCC1] | 2,3-difluoro-N-(2-(piperidin-1- yl)-4-((4- (trifluoromethyl)benzyl)amino) phenyl)heptanamide (2R,3S)-2,3-difluoro-N-(2- (piperidin-1-yl)-4-((4- (trifluoromethyl)benzyl)amino) phenyl)heptanamide (2S,3R)-2,3-difluoro-N-(2- (piperidin-1-yl)-4-((4- (trifluoromethyl)benzyl)amino) phenyl)heptanamide (2R,3R)-2,3-difluoro-N-(2- (piperidin-1-yl)-4-((4- (trifluoromethyl)benzyl)amino) |
| | phenyl)heptanamide |
| | (2S,3S)-2,3-difluoro-N-(2- |
| | (piperidin-1-yl)-4-((4- |
| | (trifluoromethyl)benzyl)amino) |
| | phenyl)heptanamide |
| 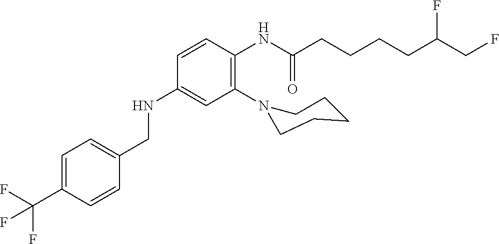 [SMILES: O=C(CCCCC(F)CF)Nc1ccc(NCc2ccc(C(F)(F)F)cc2)cc1N1CCCCC1] | 6,7-difluoro-N-(2-(piperidin-1- yl)-4-((4- (trifluoromethyl)benzyl)amino) phenyl)heptanamide (6R)-6,7-difluoro-N-(2- (piperidin-1-yl)-4-((4- (trifluoromethyl)benzyl)amino) phenyl)heptanamide (6S)-6,7-difluoro-N-(2- (piperidin-1-yl)-4-((4- (trifluoromethyl)benzyl)amino) phenyl)heptanamide |
| 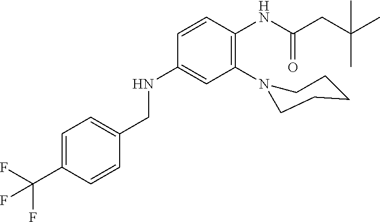 [SMILES: CC(C)(C)CC(=O)Nc1ccc(NCc2ccc(C(F)(F)F)cc2)cc1N1CCCCC1] | 3,3-dimethyl-N-(2-(piperidin-1- yl)-4-((4- (trifluoromethyl)benzyl)amino) phenyl)butanamide |
| 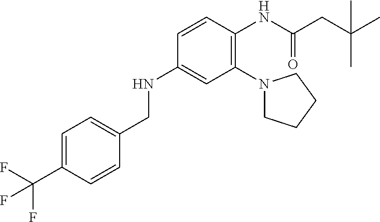 [SMILES: CC(C)(C)CC(=O)Nc1ccc(NCc2ccc(C(F)(F)F)cc2)cc1N1CCCC1] | 3,3-dimethyl-N-(2-(pyrrolidin-1- yl)-4-((4- (trifluoromethyl)benzyl)amino) phenyl)butanamide |
| 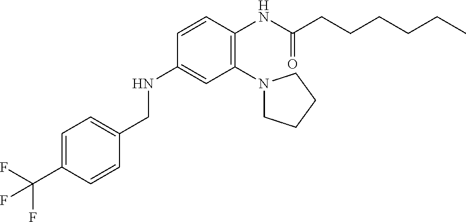 [SMILES: CCCCCCC(=O)Nc1ccc(NCc2ccc(C(F)(F)F)cc2)cc1N1CCCC1] | N-(2-(pyrrolidin-1-yl)-4-((4- (trifluoromethyl)benzyl)amino) phenyl)heptanamide |
| 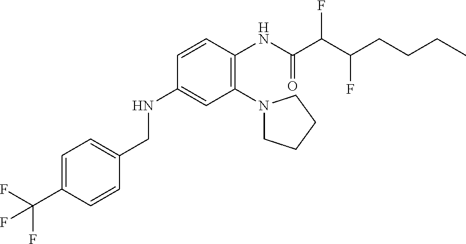 [SMILES: CCCCC(F)C(F)C(=O)Nc1ccc(NCc2ccc(C(F)(F)F)cc2)cc1N1CCCC1] | (2R,3S)-2,3-difluoro-N-(2- (pyrrolidin-1-yl)-4-((4- (trifluoromethyl)benzyl)amino) phenyl)heptanamide (2S,3R)-2,3-difluoro-N-(2- (pyrrolidin-1-yl)-4-((4- (trifluoromethyl)benzyl)amino) phenyl)heptanamide (2R,3R)-2,3-difluoro-N-(2- (pyrrolidin-1-yl)-4-((4- (trifluoromethyl)benzyl)amino) phenyl)heptanamide (2S,3S)-2,3-difluoro-N-(2- (pyrrolidin-1-yl)-4-((4- (trifluoromethyl)benzyl)amino) |
| | phenyl)heptanamide |
| 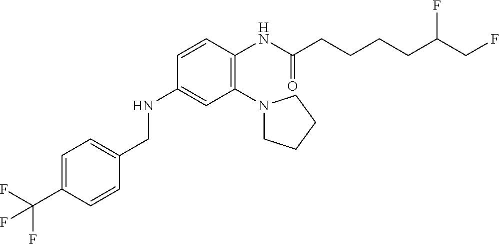 [SMILES: O=C(CCCCC(F)CF)Nc1ccc(NCc2ccc(C(F)(F)F)cc2)cc1N1CCCC1] | 6,7-difluoro-N-(2-(pyrrolidin-1- yl)-4-((4- (trifluoromethyl)benzyl)amino) phenyl)heptanamide (6R)-6,7-difluoro-N-(2- (pyrrolidin-1-yl)-4-((4- (trifluoromethyl)benzyl)amino) phenyl)heptanamide (6S)-6,7-difluoro-N-(2- (pyrrolidin-1-yl)-4-((4- (trifluoromethyl)benzyl)amino) phenyl)heptanamide |
| 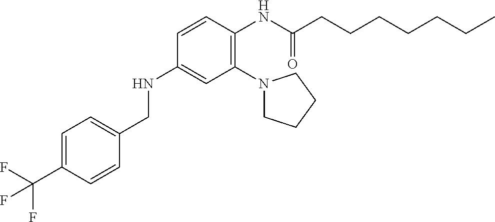 [SMILES: CCCCCCCC(=O)Nc1ccc(NCc2ccc(C(F)(F)F)cc2)cc1N1CCCC1] | N-(2-(pyrrolidin-1-yl)-4-((4- (trifluoromethyl)benzyl)amino) phenyl)octanamide |
| 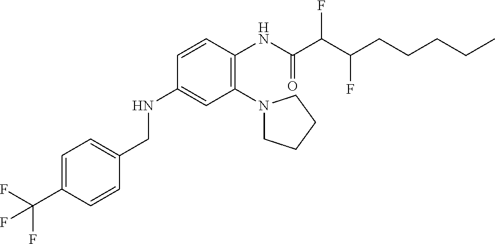 [SMILES: CCCCCC(F)C(F)C(=O)Nc1ccc(NCc2ccc(C(F)(F)F)cc2)cc1N1CCCC1] | 2,3-difluoro-N-(2-(pyrrolidin-1- yl)-4-((4- (trifluoromethyl)benzyl)amino) phenyl)octanamide (2R,3S)-2,3-difluoro-N-(2- (pyrrolidin-1-yl)-4-((4- (trifluoromethyl)benzyl)amino) phenyl)octanamide (2S,3R)-2,3-difluoro-N-(2- (pyrrolidin-1-yl)-4-((4- (trifluoromethyl)benzyl)amino) phenyl)octanamide (2R,3R)-2,3-difluoro-N-(2- (pyrrolidin-1-yl)-4-((4- (trifluoromethyl)benzyl)amino) |
| | phenyl)octanamide |
| | (2S,3S)-2,3-difluoro-N-(2- |
| | (pyrrolidin-1-yl)-4-((4- |
| | (trifluoromethyl)benzyl)amino) |
| | phenyl)octanamide |
| 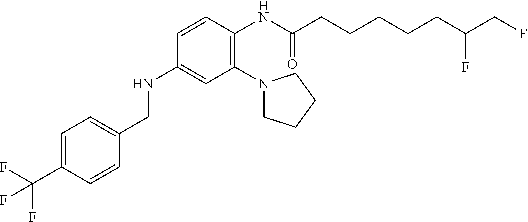 [SMILES: O=C(CCCCCC(F)CF)Nc1ccc(NCc2ccc(C(F)(F)F)cc2)cc1N1CCCC1] | 7,8-difluoro-N-(2-(pyrrolidin-1- yl)-4-((4- (trifluoromethyl)benzyl)amino) phenyl)octanamide (7R)-7,8-difluoro-N-(2- (pyrrolidin-1-yl)-4-((4- (trifluoromethyl)benzyl)amino) phenyl)octanamide (7S)-7,8-difluoro-N-(2- (pyrrolidin-1-yl)-4-((4- (trifluoromethyl)benzyl)amino) phenyl)octanamide |
| 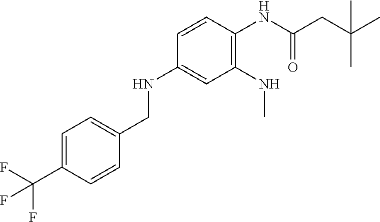 [SMILES: CNc1cc(NCc2ccc(C(F)(F)F)cc2)ccc1NC(=O)CC(C)(C)C] | 3,3-dimethyl-N-(2- (methylamino)-4-((4- (trifluoromethyl)benzyl)amino) phenyl)butanamide |
| 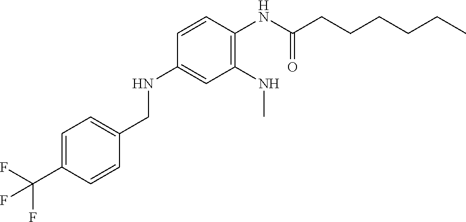 [SMILES: CCCCCCC(=O)Nc1ccc(NCc2ccc(C(F)(F)F)cc2)cc1NC] | N-(2-(methylamino)-4-((4- (trifluoromethyl)benzyl)amino) phenyl)heptanamide |
| 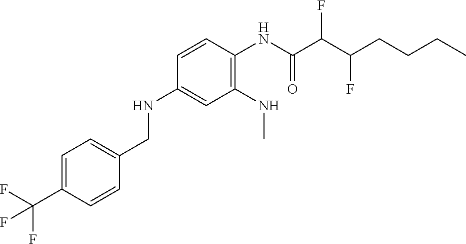 [SMILES: CCCCC(F)C(F)C(=O)Nc1ccc(NCc2ccc(C(F)(F)F)cc2)cc1NC] | 2,3-difluoro-N-(2- (methylamino)-4-((4- (trifluoromethyl)benzyl)amino) phenyl)heptanamide (2R, 3S)-2,3-difluoro-N-(2- (methylamino)-4-((4- (trifluoromethyl)benzyl)amino) phenyl)heptanamide (2S, 3R)-2,3-difluoro-N-(2- (methylamino)-4-((4- (trifluoromethyl)benzyl)amino) phenyl)heptanamide (2R, 3R)-2,3-difluoro-N-(2- (methylamino)-4-((4- (trifluoromethyl)benzyl)amino) |
| | phenyl)heptanamide |
| | (2S, 3S)-2,3-difluoro-N-(2- |
| | (methylamino)-4-((4- |
| | (trifluoromethyl)benzyl)amino) |
| | phenyl)heptanamide |
| 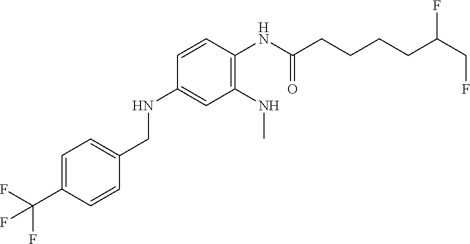 [SMILES: CNc1cc(NCc2ccc(C(F)(F)F)cc2)ccc1NC(=O)CCCCC(F)CF] | 6,7-difluoro-N-(2- (methylamino)-4-((4- (trifluoromethyl)benzyl)amino) phenyl)heptanamide (6R)-6,7-difluoro-N-(2- (methylamino)-4-((4- (trifluoromethyl)benzyl)amino) phenyl)heptanamide (6S)-6,7-difluoro-N-(2- (methylamino)-4-((4- (trifluoromethyl)benzyl)amino) phenyl)heptanamide |
| 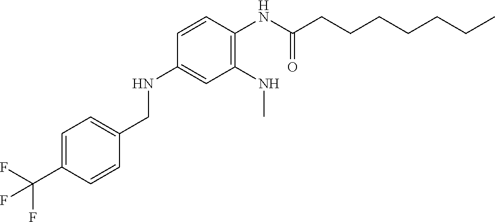 [SMILES: CCCCCCCC(=O)Nc1ccc(NCc2ccc(C(F)(F)F)cc2)cc1NC] | N-(2-(methylamino)-4-((4- (trifluoromethyl)benzyl)amino) phenyl)octanamide |
| 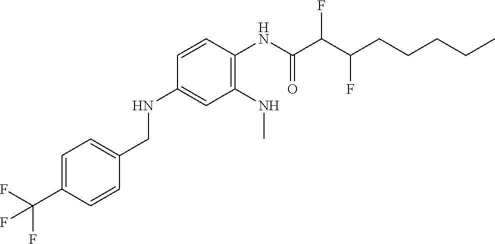 [SMILES: CCCCCC(F)C(F)C(=O)Nc1ccc(NCc2ccc(C(F)(F)F)cc2)cc1NC] | 2,3-difluoro-N-(2- (methylamino)-4-((4- (trifluoromethyl)benzyl)amino) phenyl)octanamide (2R,3S)-2,3-difluoro-N-(2- (methylamino)-4-((4- (trifluoromethyl)benzyl)amino) phenyl)octanamide (2S,3R)-2,3-difluoro-N-(2- (methylamino)-4-((4- (trifluoromethyl)benzyl)amino) phenyl)octanamide (2R,3R)-2,3-difluoro-N-(2- (methylamino)-4-((4- (trifluoromethyl)benzyl)amino) |
| | phenyl)octanamide |
| | (2S,3S)-2,3-difluoro-N-(2- |
| | (methylamino)-4-((4- |
| | (trifluoromethyl)benzyl)amino) |
| | phenyl)octanamide |
| 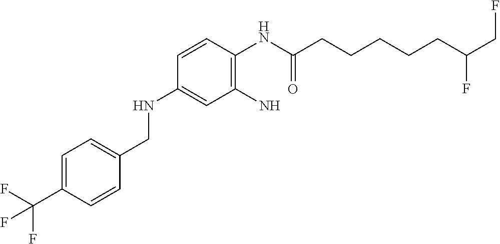 [SMILES: Nc1cc(NCc2ccc(C(F)(F)F)cc2)ccc1NC(=O)CCCCCC(F)CF] | 7,8-difluoro-N-(2- (methylamino)-4-((4- (trifluoromethyl)benzyl)amino) phenyl)octanamide (7R)-7,8-difluoro-N-(2- (methylamino)-4-((4- (trifluoromethyl)benzyl)amino) phenyl)octanamide (7S)-7,8-difluoro-N-(2- (methylamino)-4-((4- (trifluoromethyl)benzyl)amino) phenyl)octanamide |
| 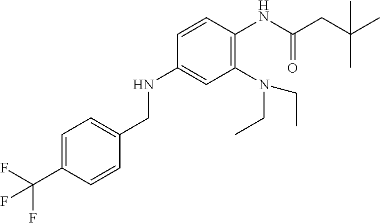 [SMILES: CCN(CC)c1cc(NCc2ccc(C(F)(F)F)cc2)ccc1NC(=O)CC(C)(C)C] | N-(2-(diethylamino)-4-((4- (trifluoromethyl)benzyl)amino) phenyl)-3,3-dimethylbutanamide |
| 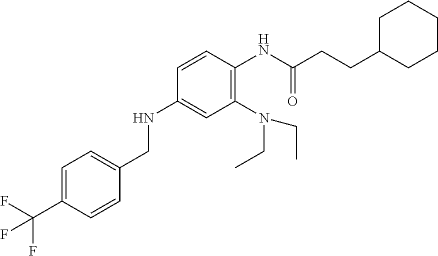 [SMILES: CCN(CC)c1cc(NCc2ccc(C(F)(F)F)cc2)ccc1NC(=O)CCC1CCCCC1] | 3-cyclohexyl-N-(2- (diethylamino)-4-((4- (trifluoromethyl)benzyl)amino) phenyl)propanamide |
| 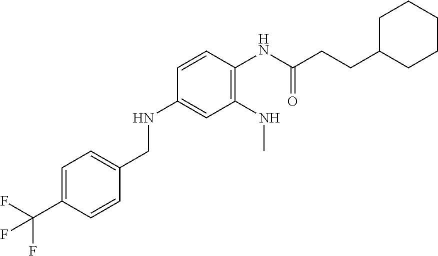 [SMILES: CNc1cc(NCc2ccc(C(F)(F)F)cc2)ccc1NC(=O)CCC1CCCCC1] | 3-cyclohexyl-N-(2- (methylamino)-4-((4- (trifluoromethyl)benzyl)amino) phenyl)propanamide |
| 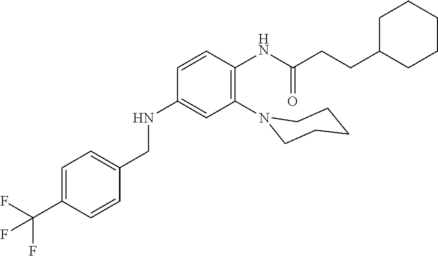 [SMILES: O=C(CCC1CCCCC1)Nc1ccc(NCc2ccc(C(F)(F)F)cc2)cc1N1CCCCC1] | 3-cyclohexyl-N-(2-(piperidin-1- yl)-4-((4- (trifluoromethyl)benzyl)amino) phenyl)propanamide |
| 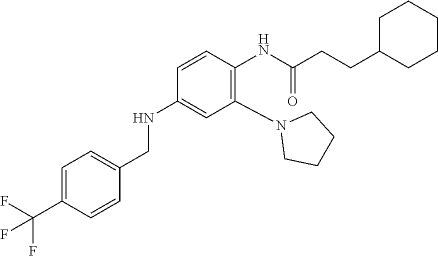 [SMILES: O=C(CCC1CCCCC1)Nc1ccc(NCc2ccc(C(F)(F)F)cc2)cc1N1CCCC1] | 3-cyclohexyl-N-(2-(pyrrolidin-1- yl)-4-((4- (trifluoromethyl)benzyl)amino) phenyl)propanamide |
| 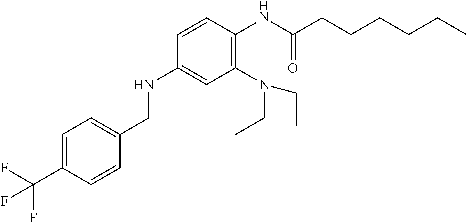 [SMILES: CCCCCCC(=O)Nc1ccc(NCc2ccc(C(F)(F)F)cc2)cc1N(CC)CC] | N-(2-(diethylamino)-4-((4- (trifluoromethyl)benzyl)amino) phenyl)heptanamide |
| 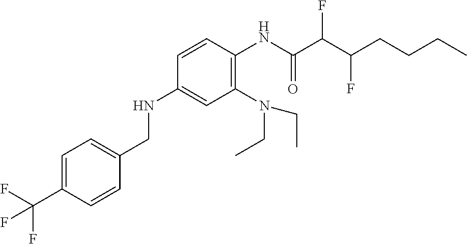 [SMILES: CCCCC(F)C(F)C(=O)Nc1ccc(NCc2ccc(C(F)(F)F)cc2)cc1N(CC)CC] | N-(2-(diethylamino)-4-((4- (trifluoromethyl)benzyl)amino) phenyl)-2,3-difluoroheptanamide (2R,3S)-N-(2-(diethylamino)-4- ((4- (trifluoromethyl)benzyl)amino) phenyl)-2,3-difluoroheptanamide (2S,3R)-N-(2-(diethylamino)-4- ((4- (trifluoromethyl)benzyl)amino) phenyl)-2,3-difluoroheptanamide (2R,3R)-N-(2-(diethylamino)-4- ((4- (trifluoromethyl)benzyl)amino) phenyl)-2,3-difluoroheptanamide |
| | (2S,3S)-N-(2-(diethylamino)-4- |
| | ((4- |
| | (trifluoromethyl)benzyl)amino) |
| | phenyl)-2,3-difluoroheptanamide. |
| 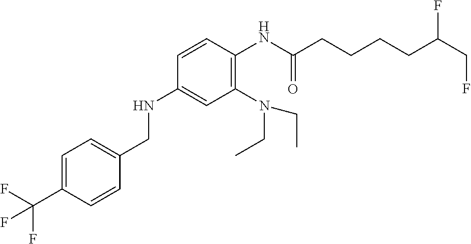 [SMILES: CCN(CC)c1cc(NCc2ccc(C(F)(F)F)cc2)ccc1NC(=O)CCCCC(F)CF] | N-(2-(diethylamino)-4-((4- (trifluoromethyl)benzyl)amino) phenyl)-6,7-difluoroheptanamide (6R)-N-(2-(diethylamino)-4-((4- (trifluoromethyl)benzyl)amino) phenyl)-6,7-difluoroheptanamide (6S)-N-(2-(diethylamino)-4-((4- (trifluoromethyl)benzyl)amino) phenyl)-6,7-difluoroheptanamide |
| 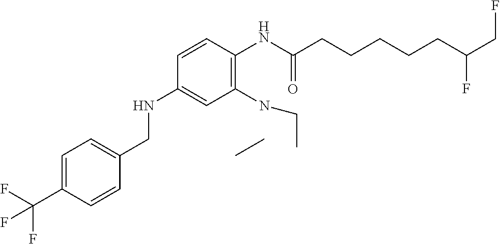 | N-(2-(diethylamino)-4-((4- (trifluoromethyl)benzyl)amino) phenyl)-7,8-difluorooctanamide (7R)-N-(2-(diethylamino)-4-((4- (trifluoromethyl)benzyl)amino) phenyl)-7,8-difluorooctanamide (7S)-N-(2-(diethylamino)-4-((4- (trifluoromethyl)benzyl)amino) phenyl)-7,8-difluorooctanamide |
| 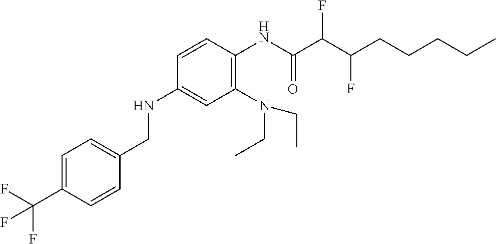 [SMILES: CCCCCC(F)C(F)C(=O)Nc1ccc(NCc2ccc(C(F)(F)F)cc2)cc1N(CC)CC] | N-(2-(diethylamino)-4-((4- (trifluoromethyl)benzyl)amino) phenyl)-2,3-difluorooctanamide (2R,3S)-N-(2-(diethylamino)-4- ((4- (trifluoromethyl)benzyl)amino) phenyl)-2,3-difluorooctanamide (2S,3R)-N-(2-(diethylamino)-4- ((4- (trifluoromethyl)benzyl)amino) phenyl)-2,3-difluorooctanamide (2R,3R)-N-(2-(diethylamino)-4- ((4- (trifluoromethyl)benzyl)amino) phenyl)-2,3-difluorooctanamide |
| | (2S,3S)-N-(2-(diethylamino)-4- |
| | ((4- |
| | (trifluoromethyl)benzyl)amino) |
| | phenyl)-2,3-difluorooctanamide |
| 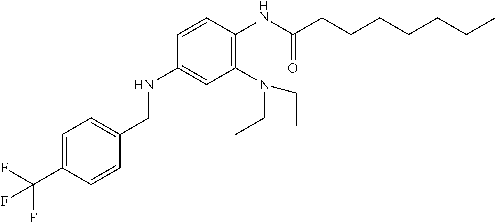 [SMILES: CCCCCCCC(=O)Nc1ccc(NCc2ccc(C(F)(F)F)cc2)cc1N(CC)CC] | N-(2-(diethylamino)-4-((4- (trifluoromethyl)benzyl)amino) phenyl)octanamide |
| 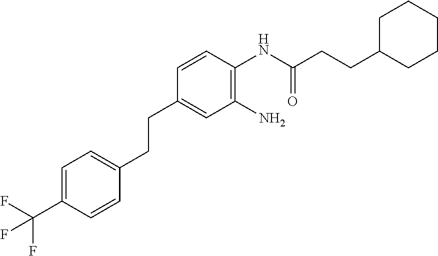 [SMILES: Nc1cc(CCc2ccc(C(F)(F)F)cc2)ccc1NC(=O)CCC1CCCCC1] | N-(2-amino-4-(4- (trifluoromethyl)phenethyl) phenyl)-3-cyclohexylpropanamide |
| 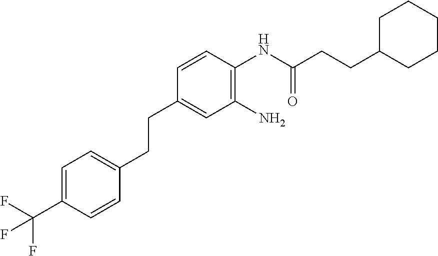 [SMILES: Nc1cc(CCc2ccc(C(F)(F)F)cc2)ccc1NC(=O)CCC1CCCCC1] | N-(2-amino-4-(4- (trifluoromethyl)phenethyl) phenyl)-3-cyclohexylpropanamide |
| 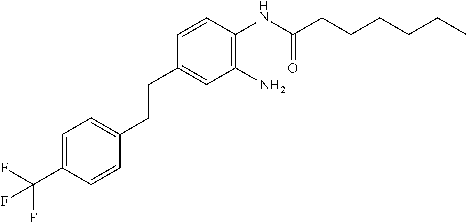 [SMILES: CCCCCCC(=O)Nc1ccc(CCc2ccc(C(F)(F)F)cc2)cc1N] | N-(2-amino-4-(4- (trifluoromethyl)phenethyl) phenyl)heptanamide |
| 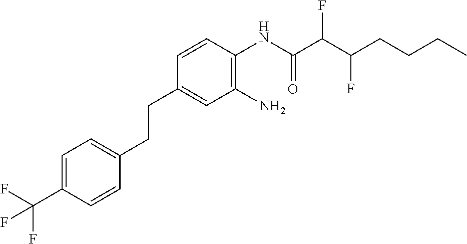 [SMILES: CCCCC(F)C(F)C(=O)Nc1ccc(CCc2ccc(C(F)(F)F)cc2)cc1N] | N-(2-amino-4-(4- (trifluoromethyl)phenethyl) phenyl)-2,3-difluoroheptanamide (2R,3S)-N-(2-amino-4-(4- (trifluoromethyl)phenethyl) phenyl)-2,3-difluoroheptanamide (2S,3R)-N-(2-amino-4-(4- (trifluoromethyl)phenethyl) phenyl)-2,3-difluoroheptanamide (2R,3R)-N-(2-amino-4-(4- (trifluoromethyl)phenethyl) phenyl)-2,3-difluoroheptanamide (2S,3S)-N-(2-amino-4-(4- (trifluoromethyl)phenethyl) phenyl)-2,3-difluoroheptanamide |
| 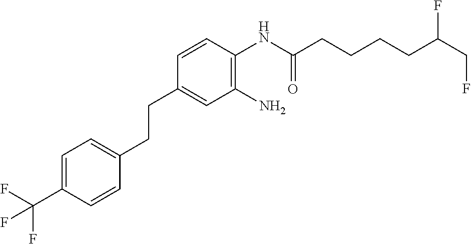 [SMILES: Nc1cc(CCc2ccc(C(F)(F)F)cc2)ccc1NC(=O)CCCCC(F)CF] | N-(2-amino-4-(4- (trifluoromethyl)phenethyl) phenyl)-6,7-difluoroheptanamide (6R)-N-(2-amino-4-(4- (trifluoromethyl)phenethyl) phenyl)-6,7-difluoroheptanamide (6S)-N-(2-amino-4-(4- (trifluoromethyl)phenethyl) phenyl)-6,7-difluoroheptanamide |
| 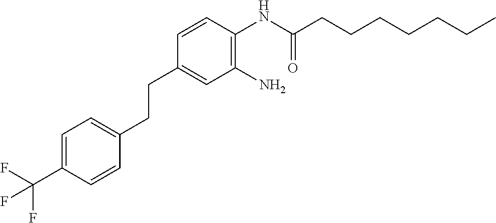 [SMILES: CCCCCCCC(=O)Nc1ccc(CCc2ccc(C(F)(F)F)cc2)cc1N] | N-(2-amino-4-(4- (trifluoromethyl)phenethyl) phenyl)octanamide |
| 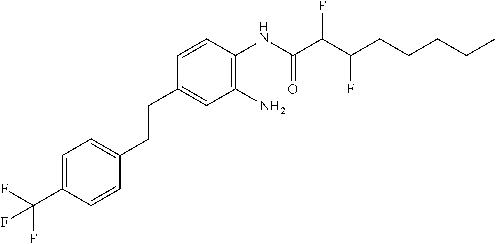 [SMILES: CCCCCC(F)C(F)C(=O)Nc1ccc(CCc2ccc(C(F)(F)F)cc2)cc1N] | N-(2-amino-4-(4- (trifluoromethyl)phenethyl) phenyl)-2,3-difluorooctanamide |
| 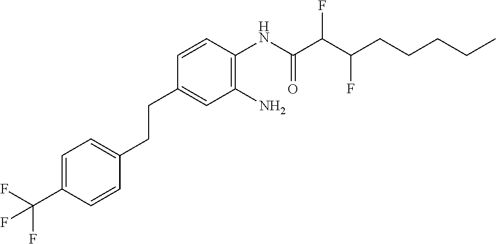 [SMILES: CCCCCC(F)C(F)C(=O)Nc1ccc(CCc2ccc(C(F)(F)F)cc2)cc1N] | N-(2-amino-4-(4- (trifluoromethyl)phenethyl) phenyl)-2,3-difluorooctanamide (2R,3S)-N-(2-amino-4-(4- (trifluoromethyl)phenethyl) phenyl)-2,3-difluorooctanamide (2S,3R)-N-(2-amino-4-(4- (trifluoromethyl)phenethyl) phenyl)-2,3-difluorooctanamide (2R,3R)-N-(2-amino-4-(4- (trifluoromethyl)phenethyl) phenyl)-2,3-difluorooctanamide (2S,3S)-N-(2-amino-4-(4- (trifluoromethyl)phenethyl) phenyl)-2,3-difluorooctanamide |
| 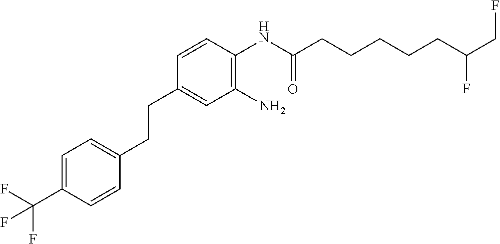 [SMILES: Nc1cc(CCc2ccc(C(F)(F)F)cc2)ccc1NC(=O)CCCCCC(F)CF] | N-(2-amino-4-(4- (trifluoromethyl)phenethyl) phenyl)-7,8-difluorooctanamide (7R)-N-(2-amino-4-(4- (trifluoromethyl)phenethyl) phenyl)-7,8-difluorooctanamide (7S)-N-(2-amino-4-(4- (trifluoromethyl)phenethyl) phenyl)-7,8-difluorooctanamide |
| 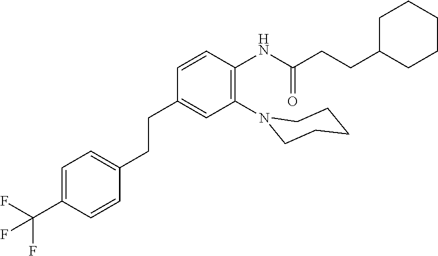 [SMILES: O=C(CCC1CCCCC1)Nc1ccc(CCc2ccc(C(F)(F)F)cc2)cc1N1CCCCC1] | 3-cyclohexyl-N-(2-(piperidin-1- yl)-4-(4- (trifluoromethyl)phenethyl) phenyl)propanamide |
| 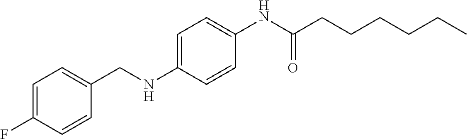 [SMILES: CCCCCCC(=O)Nc1ccc(NCc2ccc(F)cc2)cc1] | N-(4-((4- fluorobenzyl)amino)phenyl) heptanamide |
| 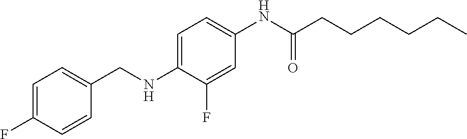 [SMILES: CCCCCCC(=O)Nc1ccc(NCc2ccc(F)cc2)c(F)c1] | N-(3-fluoro-4-((4- fluorobenzyl)amino)phenyl) heptanamide |
| 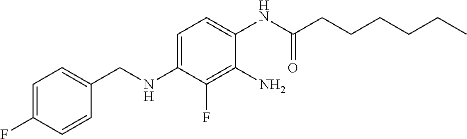 [SMILES: CCCCCCC(=O)Nc1ccc(NCc2ccc(F)cc2)c(F)c1N] | N-(2-amino-3-fluoro-4-((4- fluorobenzyl)amino)phenyl) heptanamide |
| 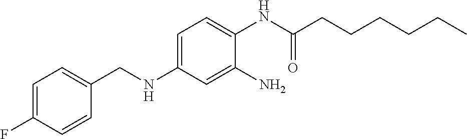 [SMILES: CCCCCCC(=O)Nc1ccc(NCc2ccc(F)cc2)cc1N] | N-(2-amino-4-((4- fluorobenzyl)amino)phenyl) heptanamide |
| 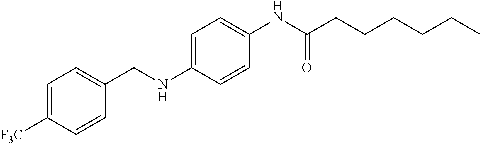 [SMILES: CCCCCCC(=O)Nc1ccc(NCc2ccc(C(F)(F)F)cc2)cc1] | N-(4-((4- (trifluoromethyl)benzyl)amino) phenyl)heptanamide |
| 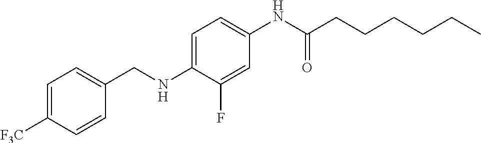 [SMILES: CCCCCCC(=O)Nc1ccc(NCc2ccc(C(F)(F)F)cc2)c(F)c1] | N-(3-fluoro-4-((4- (trifluoromethyl)benzyl)amino) phenyl)heptanamide |
| 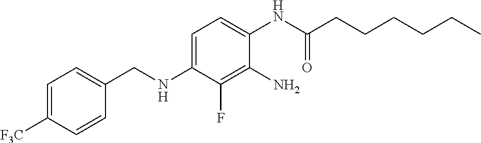 [SMILES: CCCCCCC(=O)Nc1ccc(NCc2ccc(C(F)(F)F)cc2)c(F)c1N] | N-(2-amino-3-fluoro-4-((4- (trifluoromethyl)benzyl)amino) phenyl)heptanamide |
| 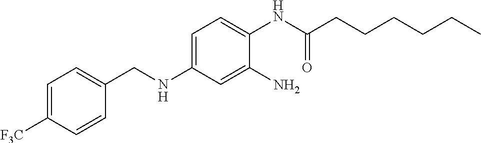 [SMILES: CCCCCCC(=O)Nc1ccc(NCc2ccc(C(F)(F)F)cc2)cc1N] | N-(2-amino-4-((4- (trifluoromethyl)benzyl)amino) phenyl)heptanamide |
| 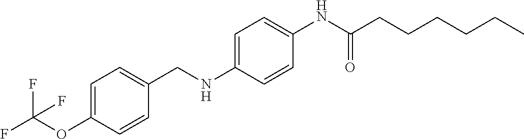 [SMILES: CCCCCCC(=O)Nc1ccc(NCc2ccc(OC(F)(F)F)cc2)cc1] | N-(4-((4- (trifluoromethoxy)benzyl)amino) phenyl)heptanamide |
| 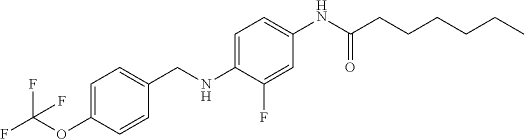 [SMILES: CCCCCCC(=O)Nc1ccc(NCc2ccc(OC(F)(F)F)cc2)c(F)c1] | N-(3-fluoro-4-((4- (trifluoromethoxy)benzyl)amino) phenyl)heptanamide |
| 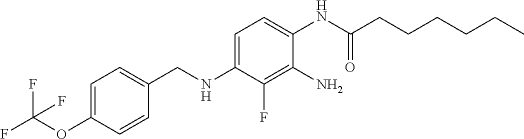 [SMILES: CCCCCCC(=O)Nc1ccc(NCc2ccc(OC(F)(F)F)cc2)c(F)c1N] | N-(2-amino-3-fluoro-4-((4- (trifluoromethoxy)benzyl)amino) phenyl)heptanamide |
| 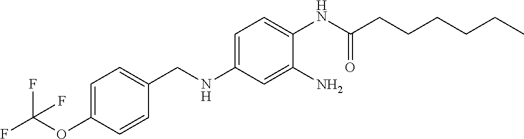 [SMILES: CCCCCCC(=O)Nc1ccc(NCc2ccc(OC(F)(F)F)cc2)cc1N] | N-(2-amino-4-((4- (trifluoromethoxy)benzyl)amino) phenyl)heptanamide |
| 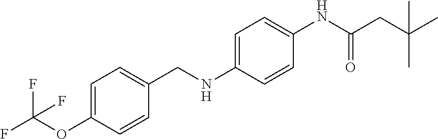 [SMILES: CC(C)(C)CC(=O)Nc1ccc(NCc2ccc(OC(F)(F)F)cc2)cc1] | 3,3-dimethyl-N-(4-((4- (trifluoromethoxy)benzyl)amino) phenyl)butanamide |
| 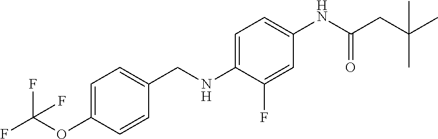 [SMILES: CC(C)(C)CC(=O)Nc1ccc(NCc2ccc(OC(F)(F)F)cc2)c(F)c1] | N-(3-fluoro-4-((4- (trifluoromethoxy)benzyl)amino) phenyl)-3,3- dimethylbutanamide |
| 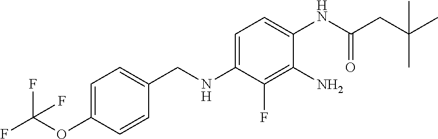 [SMILES: CC(C)(C)CC(=O)Nc1ccc(NCc2ccc(OC(F)(F)F)cc2)c(F)c1N] | N-(2-amino-3-fluoro-4-((4- (trifluoromethoxy)benzyl)amino) phenyl)-3,3- dimethylbutanamide |
| 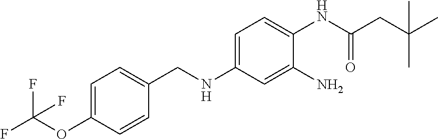 [SMILES: CC(C)(C)CC(=O)Nc1ccc(NCc2ccc(OC(F)(F)F)cc2)cc1N] | N-(2-amino-4-((4- (trifluoromethoxy)benzyl)amino) phenyl)-3,3- dimethylbutanamide |
| 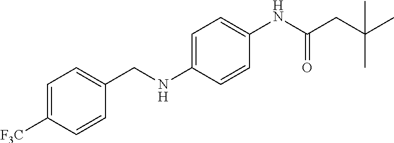 [SMILES: CC(C)(C)CC(=O)Nc1ccc(NCc2ccc(C(F)(F)F)cc2)cc1] | 3,3-dimethyl-N-(4-((4- (trifluoromethyl)benzyl)amino) phenyl)butanamide |
| 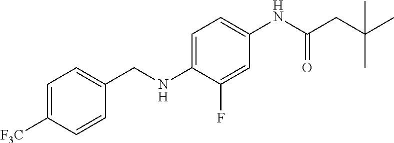 [SMILES: CC(C)(C)CC(=O)Nc1ccc(NCc2ccc(C(F)(F)F)cc2)c(F)c1] | N-(3-fluoro-4-((4- (trifluoromethyl)benzyl)amino) phenyl)-3,3-dimethylbutanamide |
| 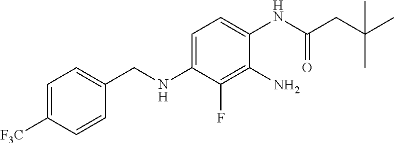 [SMILES: CC(C)(C)CC(=O)Nc1ccc(NCc2ccc(C(F)(F)F)cc2)c(F)c1N] | N-(2-amino-3-fluoro-4-((4- (trifluoromethyl)benzyl)amino) phenyl)-3,3-dimethylbutanamide |
| 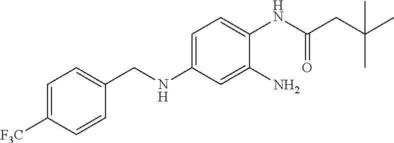 [SMILES: CC(C)(C)CC(=O)Nc1ccc(NCc2ccc(C(F)(F)F)cc2)cc1N] | N-(2-amino-4-((4- (trifluoromethyl)benzyl)amino) phenyl)-3,3-dimethylbutanamide |
| 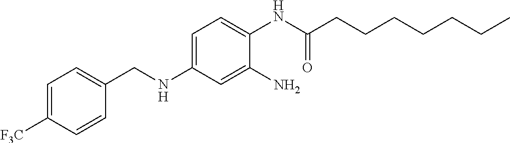 [SMILES: CCCCCCCC(=O)Nc1ccc(NCc2ccc(C(F)(F)F)cc2)cc1N] | N-(2-amino-4-((4- (trifluoromethyl)benzyl)amino) phenyl)octanamide |
| 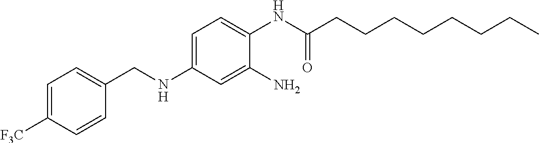 [SMILES: CCCCCCCCC(=O)Nc1ccc(NCc2ccc(C(F)(F)F)cc2)cc1N] | N-(2-amino-4-((4- (trifluoromethyl)benzyl)amino) phenyl)nonanamide |
| 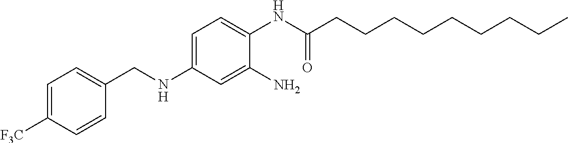 [SMILES: CCCCCCCCCC(=O)Nc1ccc(NCc2ccc(C(F)(F)F)cc2)cc1N] | N-(2-amino-4-((4- (trifluoromethyl)benzyl)amino) phenyl)decanamide |
| 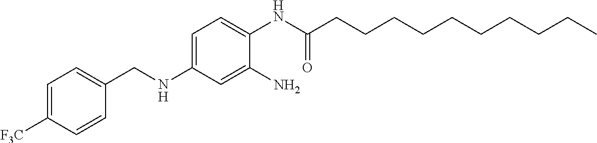 [SMILES: CCCCCCCCCCC(=O)Nc1ccc(NCc2ccc(C(F)(F)F)cc2)cc1N] | N-(2-amino-4-((4- (trifluoromethyl)benzyl)amino) phenyl)undecanamide |
| 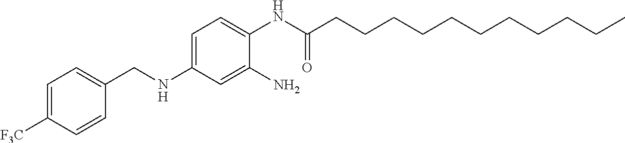 [SMILES: CCCCCCCCCCCC(=O)Nc1ccc(NCc2ccc(C(F)(F)F)cc2)cc1N] | N-(2-amino-4-((4- (trifluoromethyl)benzyl)amino) phenyl)dodecanamide |
| 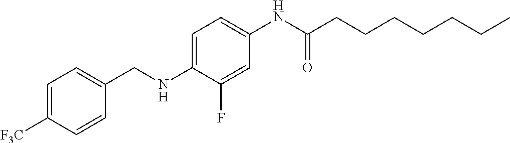 [SMILES: CCCCCCCC(=O)Nc1ccc(NCc2ccc(C(F)(F)F)cc2)c(F)c1] | N-(3-fluoro-4-((4- (trifluoromethyl)benzyl)amino) phenyl)octanamide |
| 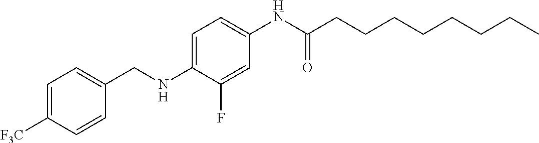 [SMILES: CCCCCCCCC(=O)Nc1ccc(NCc2ccc(C(F)(F)F)cc2)c(F)c1] | N-(3-fluoro-4-((4- (trifluoromethyl)benzyl)amino) phenyl)nonanamide |
| 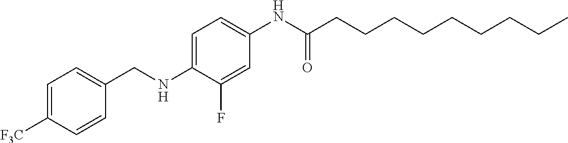 [SMILES: CCCCCCCCCC(=O)Nc1ccc(NCc2ccc(C(F)(F)F)cc2)c(F)c1] | N-(3-fluoro-4-((4- (trifluoromethyl)benzyl)amino) phenyl)decanamide |
| 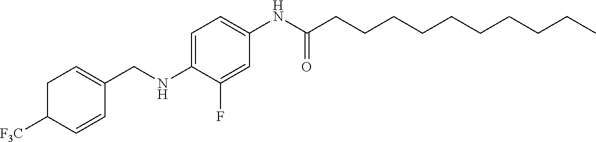 [SMILES: CCCCCCCCCCC(=O)Nc1ccc(NCC2=CCC(C(F)(F)F)C=C2)c(F)c1] | N-(3-fluoro-4-((4- (trifluoromethyl)benzyl)amino) phenyl)undecanamide |
| 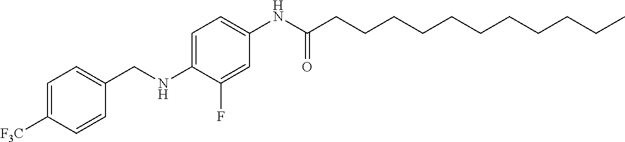 [SMILES: CCCCCCCCCCCC(=O)Nc1ccc(NCc2ccc(C(F)(F)F)cc2)c(F)c1] | N-(3-fluoro-4-((4- (trifluoromethyl)benzyl)amino) phenyl)dodecanamide |
| 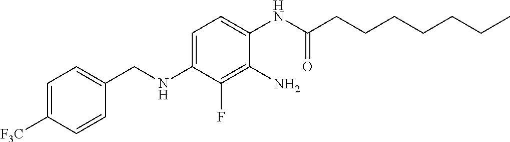 [SMILES: CCCCCCCC(=O)Nc1ccc(NCc2ccc(C(F)(F)F)cc2)c(F)c1N] | N-(2-amino-3-fluoro-4-((4- (trifluoromethyl)benzyl)amino) phenyl)octanamide |
| 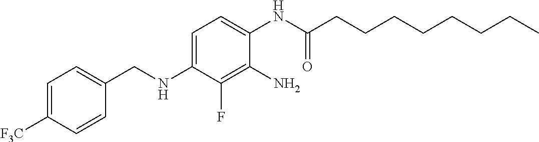 [SMILES: CCCCCCCCC(=O)Nc1ccc(NCc2ccc(C(F)(F)F)cc2)c(F)c1N] | N-(2-amino-3-fluoro-4-((4- (trifluoromethyl)benzyl)amino) phenyl)nonanamide |
| 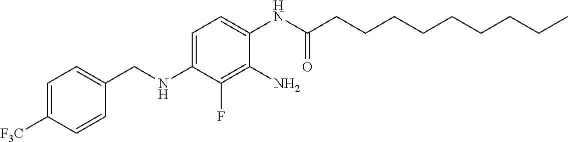 [SMILES: CCCCCCCCCC(=O)Nc1ccc(NCc2ccc(C(F)(F)F)cc2)c(F)c1N] | N-(2-amino-3-fluoro-4-((4- (trifluoromethyl)benzyl)amino) phenyl)decanamide |
| 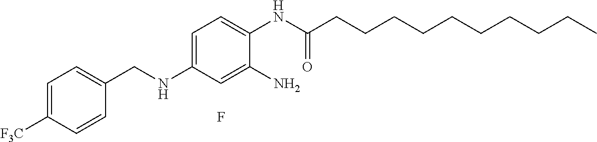 | N-(2-amino-3-fluoro-4-((4- (trifluoromethyl)benzyl)amino) phenyl)undecanamide |
| 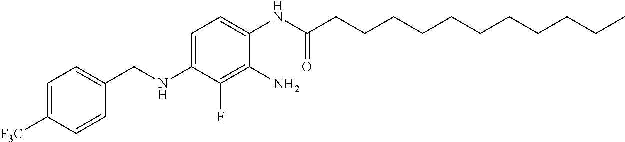 [SMILES: CCCCCCCCCCCC(=O)Nc1ccc(NCc2ccc(C(F)(F)F)cc2)c(F)c1N] | N-(2-amino-3-fluoro-4-((4- (trifluoromethyl)benzyl)amino) phenyl)dodecanamide |
| 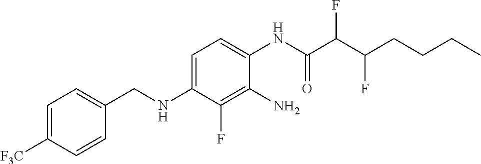 [SMILES: CCCCC(F)C(F)C(=O)Nc1ccc(NCc2ccc(C(F)(F)F)cc2)c(F)c1N] | N-(2-amino-3-fluoro-4-((4- (trifluoromethyl)benzyl)amino) phenyl)-2,3-difluoroheptanamide (2R,3S)-N-(2-amino-3-fluoro-4- ((4- (trifluoromethyl)benzyl)amino) phenyl)-2,3-difluoroheptanamide (2S,3R)-N-(2-amino-3-fluoro-4- ((4- (trifluoromethyl)benzyl)amino) phenyl)-2,3-difluoroheptanamide |
| | (2R,3R)-N-(2-amino-3-fluoro-4- |
| | ((4- |
| | (trifluoromethyl)benzyl)amino) |
| | phenyl)-2,3-difluoroheptanamide |
| | (2S,3S)-N-(2-amino-3-fluoro-4- |
| | ((4- |
| | (trifluoromethyl)benzyl)amino) |
| | phenyl)-2,3-difluoroheptanamide |
| | racemic mixtures thereof |
| 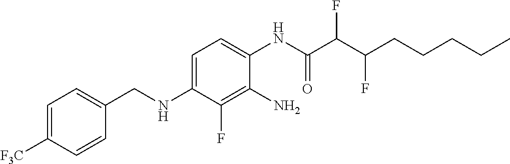 [SMILES: CCCCCC(F)C(F)C(=O)Nc1ccc(NCc2ccc(C(F)(F)F)cc2)c(F)c1N] | N-(2-amino-3-fluoro-4-((4- (trifluoromethyl)benzyl)amino) phenyl)-2,3-difluorooctanamide (2R,3S)-N-(2-amino-3-fluoro-4- ((4- (trifluoromethyl)benzyl)amino) phenyl)-2,3-difluorooctanamide (2S,3R)-N-(2-amino-3-fluoro-4- ((4- (trifluoromethyl)benzyl)amino) |
| | phenyl)-2,3-difluorooctanamide |
| | (2R,3R)-N-(2-amino-3-fluoro-4- |
| | ((4- |
| | (trifluoromethyl)benzyl)amino) |
| | phenyl)-2,3-difluorooctanamide |
| | (2S,3S)-N-(2-amino-3-fluoro-4- |
| | ((4- |
| | (trifluoromethyl)benzyl)amino) |
| | phenyl)-2,3-difluorooctanamide |
| | racemic mixtures thereof |
| 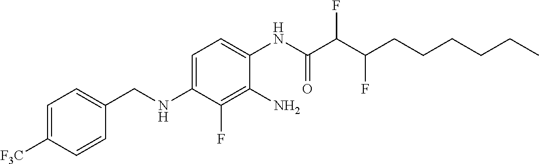 [SMILES: CCCCCCC(F)C(F)C(=O)Nc1ccc(NCc2ccc(C(F)(F)F)cc2)c(F)c1N] | N-(2-amino-3-fluoro-4-((4- (trifluoromethyl)benzyl)amino) phenyl)-2,3-difluorononanamide (2R,3S)-N-(2-amino-3-fluoro-4- ((4- (trifluoromethyl)benzyl)amino) phenyl)-2,3-difluorononanamide (2S,3R)-N-(2-amino-3-fluoro-4- ((4- (trifluoromethyl)benzyl)amino) |
| | phenyl)-2,3-difluorononanamide |
| | (2R,3R)-N-(2-amino-3-fluoro-4- |
| | ((4- |
| | (trifluoromethyl)benzyl)amino) |
| | phenyl)-2,3-difluorononanamide |
| | (2S,3S)-N-(2-amino-3-fluoro-4- |
| | ((4- |
| | (trifluoromethyl)benzyl)amino) |
| | phenyl)-2,3-difluorononanamide |
| | racemic mixtures thereof |
| 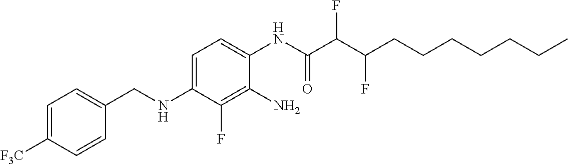 [SMILES: CCCCCCCC(F)C(F)C(=O)Nc1ccc(NCc2ccc(C(F)(F)F)cc2)c(F)c1N] | N-(2-amino-3-fluoro-4-((4- (trifluoromethyl)benzyl)amino) phenyl)-2,3-difluorodecanamide (2R,3S)-N-(2-amino-3-fluoro-4- ((4- (trifluoromethyl)benzyl)amino) phenyl)-2,3-difluorodecanamide (2S,3R)-N-(2-amino-3-fluoro-4- ((4- (trifluoromethyl)benzyl)amino) |
| | phenyl)-2,3-difluorodecanamide |
| | (2R,3R)-N-(2-amino-3-fluoro-4- |
| | ((4- |
| | (trifluoromethyl)benzyl)amino) |
| | phenyl)-2,3-difluorodecanamide |
| | (2S,3S)-N-(2-amino-3-fluoro-4- |
| | ((4- |
| | (trifluoromethyl)benzyl)amino) |
| | phenyl)-2,3-difluorodecanamide |
| | racemic mixtures thereof |
| 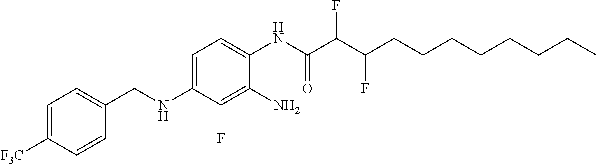 | N-(2-amino-3-fluoro-4-((4- (trifluoromethyl)benzyl)amino) phenyl)-2,3- difluoroundecanamide (2R,3S)-N-(2-amino-3-fluoro-4- ((4- (trifluoromethyl)benzyl)amino) phenyl)-2,3- difluoroundecanamide (2S,3R)-N-(2-amino-3-fluoro-4- ((4- |
| | (trifluoromethyl)benzyl)amino) |
| | phenyl)-2,3- |
| | difluoroundecanamide |
| | (2R,3R)-N-(2-amino-3-fluoro-4- |
| | ((4- |
| | (trifluoromethyl)benzyl)amino) |
| | phenyl)-2,3- |
| | difluoroundecanamide |
| | (2S,3S)-N-(2-amino-3-fluoro-4- |
| | ((4- |
| | (trifluoromethyl)benzyl)amino) |
| | phenyl)-2,3- |
| | difluoroundecanamide |
| | racemic mixtures thereof |
| 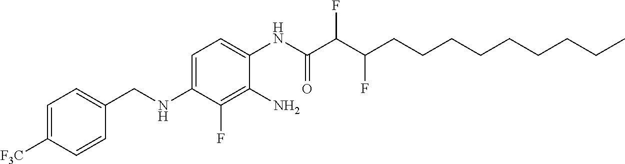 [SMILES: CCCCCCCCCC(F)C(F)C(=O)Nc1ccc(NCc2ccc(C(F)(F)F)cc2)c(F)c1N] | N-(2-amino-3-fluoro-4-((4- (trifluoromethyl)benzyl)amino) phenyl)-2,3- difluorododecanamide (2R,3S)-N-(2-amino-3-fluoro-4- ((4- (trifluoromethyl)benzyl)amino) phenyl)-2,3- difluorododecanamide (2S,3R)-N-(2-amino-3-fluoro-4- ((4- |
| | (trifluoromethyl)benzyl)amino) |
| | phenyl)-2,3- |
| | difluorododecanamide |
| | (2R,3R)-N-(2-amino-3-fluoro-4- |
| | ((4- |
| | (trifluoromethyl)benzyl)amino) |
| | phenyl)-2,3- |
| | difluorododecanamide |
| | (2S,3S)-N-(2-amino-3-fluoro-4- |
| | ((4- |
| | (trifluoromethyl)benzyl)amino) |
| | phenyl)-2,3- |
| | difluorododecanamide |
| | racemic mixtures thereof |
| 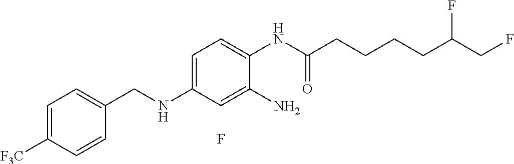 | N-(2-amino-3-fluoro-4-((4- (trifluoromethyl)benzyl)amino) phenyl)-6,7-difluoroheptanamide (6R)-N-(2-amino-3-fluoro-4-((4- (trifluoromethyl)benzyl)amino) phenyl)-6,7-difluoroheptanamide (6S)-N-(2-amino-3-fluoro-4-((4- (trifluoromethyl)benzyl)amino) phenyl)-6,7-difluoroheptanamide racemic mixtures thereof |
| 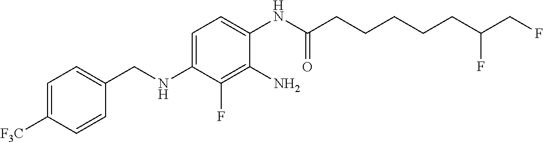 [SMILES: Nc1c(NC(=O)CCCCCC(F)CF)ccc(NCc2ccc(C(F)(F)F)cc2)c1F] | N-(2-amino-3-fluoro-4-((4- (trifluoromethyl)benzyl)amino) phenyl)-6,7-difluorooctanamide (6R)-N-(2-amino-3-fluoro-4-((4- (trifluoromethyl)benzyl)amino) phenyl)-6,7-difluorooctanamide (6S)-N-(2-amino-3-fluoro-4-((4- (trifluoromethyl)benzyl)amino) phenyl)-6,7-difluorooctanamide |
| | racemic mixtures thereof |
| 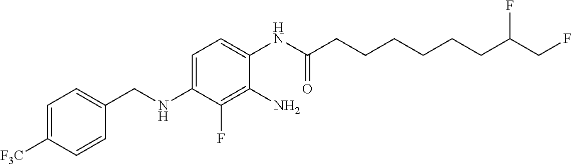 [SMILES: Nc1c(NC(=O)CCCCCCC(F)CF)ccc(NCc2ccc(C(F)(F)F)cc2)c1F] | N-(2-amino-3-fluoro-4-((4- (trifluoromethyl)benzyl)amino) phenyl)-8,9-difluorononanamide (8R)-N-(2-amino-3-fluoro-4-((4- (trifluoromethyl)benzyl)amino) phenyl)-8,9-difluorononanamide (8S)-N-(2-amino-3-fluoro-4-((4- (trifluoromethyl)benzyl)amino) phenyl)-8,9-difluorononanamide racemic mixtures thereof |
| 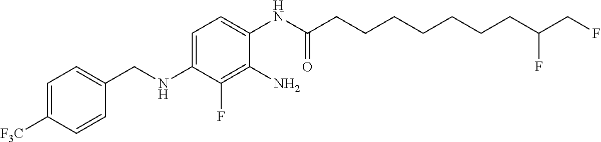 [SMILES: Nc1c(NC(=O)CCCCCCCC(F)CF)ccc(NCc2ccc(C(F)(F)F)cc2)c1F] | N-(2-amino-3-fluoro-4-((4- (trifluoromethyl)benzyl)amino) phenyl)-9,10-difluorodecanamide (9R)-N-(2-amino-3-fluoro-4-((4- (trifluoromethyl)benzyl)amino) phenyl)-9,10-difluorodecanamide (9S)-N-(2-amino-3-fluoro-4-((4- (trifluoromethyl)benzyl)amino) phenyl)-9,10-difluorodecanamide |
| | racemic mixtures thereof |
| 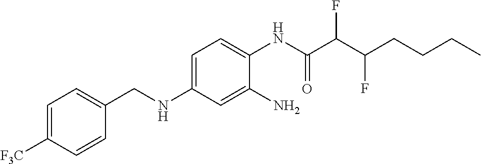 [SMILES: CCCCC(F)C(F)C(=O)Nc1ccc(NCc2ccc(C(F)(F)F)cc2)cc1N] | N-(2-amino-4-((4- (trifluoromethyl)benzyl)amino) phenyl)-2,3-difluoroheptanamide (2R,3S)-N-(2-amino-4-((4- (trifluoromethyl)benzyl)amino) phenyl)-2,3-difluoroheptanamide (2S,3R)-N-(2-amino-4-((4- (trifluoromethyl)benzyl)amino) phenyl)-2,3-difluoroheptanamide (2R,3R)-N-(2-amino-4-((4- |
| | (trifluoromethyl)benzyl)amino) |
| | phenyl)-2,3-difluoroheptanamide |
| | (2S,3S)-N-(2-amino-4-((4- |
| | (trifluoromethyl)benzyl)amino) |
| | phenyl)-2,3-difluoroheptanamide |
| | racemic mixtures thereof |
| 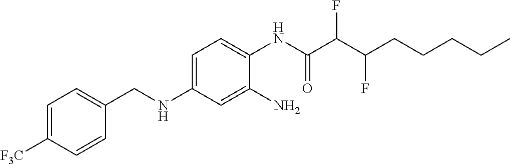 [SMILES: CCCCCC(F)C(F)C(=O)Nc1ccc(NCc2ccc(C(F)(F)F)cc2)cc1N] | N-(2-amino-4-((4- (trifluoromethyl)benzyl)amino) phenyl)-2,3-difluorooctanamide (2R,3S)-N-(2-amino-4-((4- (trifluoromethyl)benzyl)amino) phenyl)-2,3-difluorooctanamide (2S,3R)-N-(2-amino-4-((4- (trifluoromethyl)benzyl)amino) phenyl)-2,3-difluorooctanamide (2R,3R)-N-(2-amino-4-((4- (trifluoromethyl)benzyl)amino) |
| | phenyl)-2,3-difluorooctanamide |
| | (2S,3S)-N-(2-amino-4-((4- |
| | (trifluoromethyl)benzyl)amino) |
| | phenyl)-2,3-difluorooctanamide |
| | racemic mixtures thereof |
| 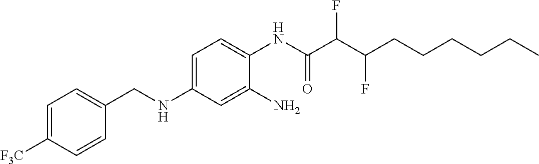 [SMILES: CCCCCCC(F)C(F)C(=O)Nc1ccc(NCc2ccc(C(F)(F)F)cc2)cc1N] | N-(2-amino-4-((4- (trifluoromethyl)benzyl)amino) phenyl)-2,3-difluorononanamide (2R,3S)-N-(2-amino-4-((4- (trifluoromethyl)benzyl)amino) phenyl)-2,3-difluorononanamide (2S,3R)-N-(2-amino-4-((4- (trifluoromethyl)benzyl)amino) phenyl)-2,3-difluorononanamide (2R,3R)-N-(2-amino-4-((4- |
| | (trifluoromethyl)benzyl)amino) |
| | phenyl)-2,3-difluorononanamide |
| | (2S,3S)-N-(2-amino-4-((4- |
| | (trifluoromethyl)benzyl)amino) |
| | phenyl)-2,3-difluorononanamide |
| | racemic mixtures thereof |
| 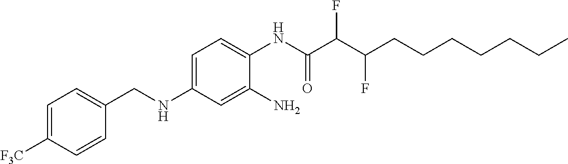 [SMILES: CCCCCCCC(F)C(F)C(=O)Nc1ccc(NCc2ccc(C(F)(F)F)cc2)cc1N] | N-(2-amino-4-((4- (trifluoromethyl)benzyl)amino) phenyl)-2,3-difluorodecanamide (2R,3S)-N-(2-amino-4-((4- (trifluoromethyl)benzyl)amino) phenyl)-2,3-difluorodecanamide (2S,3R)-N-(2-amino-4-((4- (trifluoromethyl)benzyl)amino) phenyl)-2,3-difluorodecanamide (2R,3R)-N-(2-amino-4-((4- |
| | (trifluoromethyl)benzyl)amino) |
| | phenyl)-2,3-difluorodecanamide |
| | (2S,3S)-N-(2-amino-4-((4- |
| | (trifluoromethyl)benzyl)amino) |
| | phenyl)-2,3-difluorodecanamide |
| | racemic mixtures thereof |
| 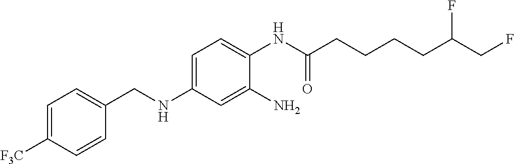 [SMILES: Nc1cc(NCc2ccc(C(F)(F)F)cc2)ccc1NC(=O)CCCCC(F)CF] | N-(2-amino-4-((4- (trifluoromethyl)benzyl)amino) phenyl)-6,7-difluoroheptanamide (6R)-N-(2-amino-4-((4- (trifluoromethyl)benzyl)amino) phenyl)-6,7-difluoroheptanamide (6S)-N-(2-amino-4-((4- (trifluoromethyl)benzyl)amino) phenyl)-6,7-difluoroheptanamide racemic mixtures thereof |
| 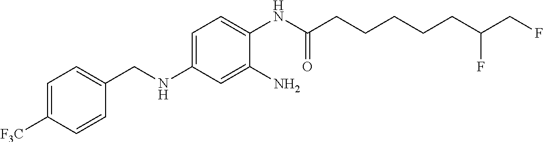 [SMILES: Nc1cc(NCc2ccc(C(F)(F)F)cc2)ccc1NC(=O)CCCCCC(F)CF] | N-(2-amino-4-((4- (trifluoromethyl)benzyl)amino) phenyl)-7,8-difluorooctanamide (7R)-N-(2-amino-4-((4- (trifluoromethyl)benzyl)amino) phenyl)-7,8-difluorooctanamide (7S)-N-(2-amino-4-((4- (trifluoromethyl)benzyl)amino) phenyl)-7,8-difluorooctanamide |
| | racemic mixtures thereof |
| 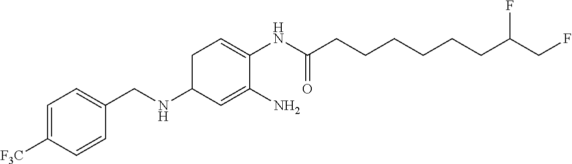 [SMILES: NC1=CC(NCc2ccc(C(F)(F)F)cc2)CC=C1NC(=O)CCCCCCC(F)CF] | N-(2-amino-3-fluoro-4-((4- (trifluoromethyl)benzyl)amino) phenyl)-6,7-difluorononanamide (6R)-N-(2-amino-3-fluoro-4-((4- (trifluoromethyl)benzyl)amino) phenyl)-6,7-difluorononanamide (6S)-N-(2-amino-3-fluoro-4-((4- (trifluoromethyl)benzyl)amino) phenyl)-6,7-difluorononanamide racemic mixtures thereof |
| 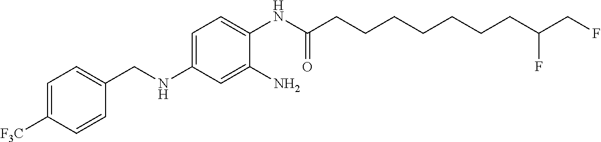 [SMILES: Nc1cc(NCc2ccc(C(F)(F)F)cc2)ccc1NC(=O)CCCCCCCC(F)CF] | N-(2-amino-3-fluoro-4-((4- (trifluoromethyl)benzyl)amino) phenyl)-6,7- difluorododecanamide (6R)-N-(2-amino-3-fluoro-4-((4- (trifluoromethyl)benzyl)amino) phenyl)-6,7- difluorododecanamide (6S)-N-(2-amino-3-fluoro-4-((4- |
| | (trifluoromethyl)benzyl)amino) |
| | phenyl)-6,7- |
| | difluorododecanamide |
| | racemic mixtures thereof |
| 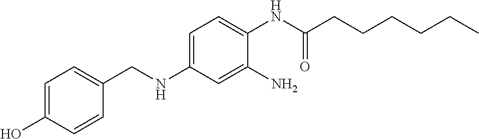 [SMILES: CCCCCCC(=O)Nc1ccc(NCc2ccc(O)cc2)cc1N] | N-(2-amino-4-((4- hydroxybenzyl)amino)phenyl) heptanamide |
| 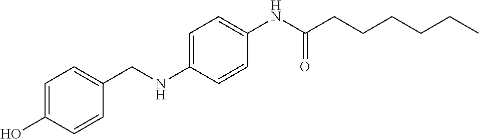 [SMILES: CCCCCCC(=O)Nc1ccc(NCc2ccc(O)cc2)cc1] | N-(4-((4- hydroxybenzyl)amino)phenyl) heptanamide |
| 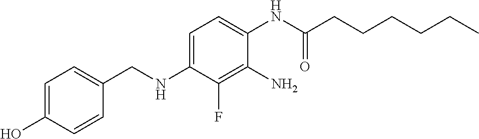 [SMILES: CCCCCCC(=O)Nc1ccc(NCc2ccc(O)cc2)c(F)c1N] | N-(2-amino-3-fluoro-4-((4- hydroxybenzyl)amino)phenyl) heptanamide |
| 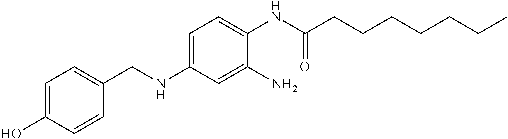 [SMILES: CCCCCCCC(=O)Nc1ccc(NCc2ccc(O)cc2)cc1N] | N-(2-amino-4-((4- hydroxybenzyl)amino)phenyl) octanamide |
| 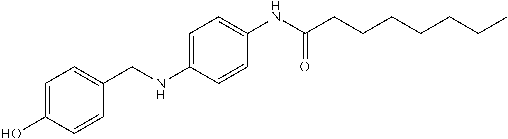 [SMILES: CCCCCCCC(=O)Nc1ccc(NCc2ccc(O)cc2)cc1] | N-(4-((4- hydroxybenzyl)amino)phenyl) octanamide |
| 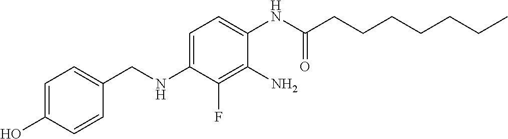 [SMILES: CCCCCCCC(=O)Nc1ccc(NCc2ccc(O)cc2)c(F)c1N] | N-(2-amino-3-fluoro-4-((4- hydroxybenzyl)amino)phenyl) octanamide |
| 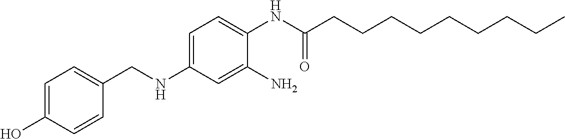 [SMILES: CCCCCCCCCC(=O)Nc1ccc(NCc2ccc(O)cc2)cc1N] | N-(2-amino-4-((4- hydroxybenzyl)amino)phenyl) decanamide |
| 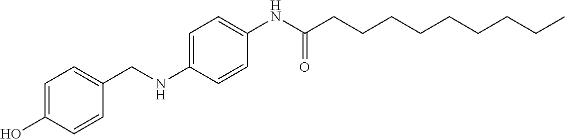 [SMILES: CCCCCCCCCC(=O)Nc1ccc(NCc2ccc(O)cc2)cc1] | N-(4-((4- hydroxybenzyl)amino)phenyl) decanamide |
| 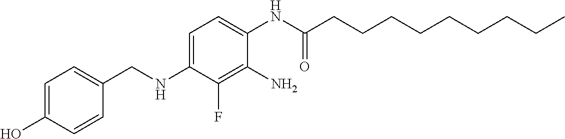 [SMILES: CCCCCCCCCC(=O)Nc1ccc(NCc2ccc(O)cc2)c(F)c1N] | N-(2-amino-3-fluoro-4-((4- hydroxybenzyl)amino)phenyl) decanamide |
| 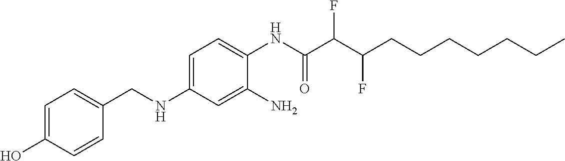 [SMILES: CCCCCCCC(F)C(F)C(=O)Nc1ccc(NCc2ccc(O)cc2)cc1N] | N-(2-amino-4-((4- hydroxybenzyl)amino)phenyl)- 2,3-difluorodecanamide (2R,3S)-N-(2-amino-4-((4- hydroxybenzyl)amino)phenyl)- 2,3-difluorodecanamide (2S,3R)-N-(2-amino-4-((4- hydroxybenzyl)amino)phenyl)- 2,3-difluorodecanamide (2R,3R)-N-(2-amino-4-((4- |
| | hydroxybenzyl)amino)phenyl)- |
| | 2,3-difluorodecanamide |
| | (2S,3S)-N-(2-amino-4-((4- |
| | hydroxybenzyl)amino)phenyl)- |
| | 2,3-difluorodecanamide |
| | racemic mixtures thereof |
| 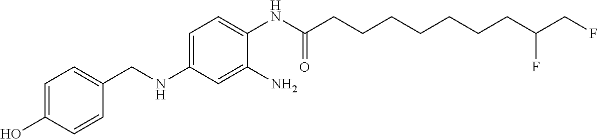 [SMILES: Nc1cc(NCc2ccc(O)cc2)ccc1NC(=O)CCCCCCCC(F)CF] | N-(2-amino-4-((4- hydroxybenzyl)amino)phenyl)- 9,10-difluorodecanamide (9R)-N-(2-amino-4-((4- hydroxybenzyl)amino)phenyl)- 2,3-difluorodecanamide (9S)-N-(2-amino-4-((4- hydroxybenzyl)amino)phenyl)- 2,3-difluorodecanamide |
| | racemic mixtures thereof |
| 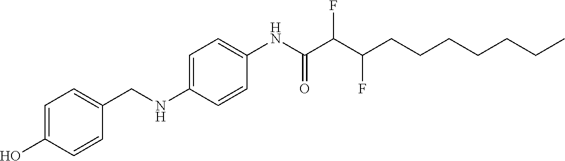 [SMILES: CCCCCCCC(F)C(F)C(=O)Nc1ccc(NCc2ccc(O)cc2)cc1] | 2,3-difluoro-N-(4-((4- hydroxybenzyl)amino)phenyl) deca decanamide (2R,3S)-2,3-difluoro-N-(4-((4- hydroxybenzyl)amino)phenyl) deca decanamide (2S,3R)-2,3-difluoro-N-(4-((4- hydroxybenzyl)amino)phenyl) decanamide (2R,3R)-2,3-difluoro-N-(4-((4- |
| | hydroxybenzyl)amino)phenyl) |
| | deca decanamide |
| | (2S,3S)-2,3-difluoro-N-(4-((4- |
| | hydroxybenzyl)amino)phenyl) |
| | decanamide |
| | racemic mixtures thereof |
| 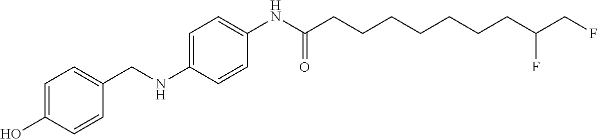 [SMILES: O=C(CCCCCCCC(F)CF)Nc1ccc(NCc2ccc(O)cc2)cc1] | 9,10-difluoro-N-(4-((4- hydroxybenzyl)amino)phenyl) decanamide (9R)-9,10-difluoro-N-(4-((4- hydroxybenzyl)amino)phenyl) decanamide (9S)-9,10-difluoro-N-(4-((4- hydroxybenzyl)amino)phenyl) deca namide |
| | racemic mixtures thereof |
| 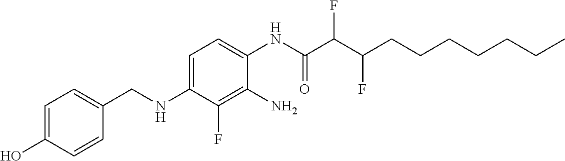 [SMILES: CCCCCCCC(F)C(F)C(=O)Nc1ccc(NCc2ccc(O)cc2)c(F)c1N] | N-(2-amino-3-fluoro-4-((4- hydroxybenzyl)amino)phenyl)- 2,3-difluorodecanamide (2R,3S)-N-(2-amino-3-fluoro-4- ((4- hydroxybenzyl)amino)phenyl)- 2,3-difluorodecanamide (2S,3R)-N-(2-amino-3-fluoro-4- ((4- hydroxybenzyl)amino)phenyl)- |
| | 2,3-difluorodecanamide |
| | (2R,3R)-N-(2-amino-3-fluoro-4- |
| | ((4- |
| | hydroxybenzyl)amino)phenyl)- |
| | 2,3-difluorodecanamide |
| | (2S,3S)-N-(2-amino-3-fluoro-4- |
| | ((4- |
| | hydroxybenzyl)amino)phenyl)- |
| | 2,3-difluorodecanamide |
| | racemic mixtures thereof |
| 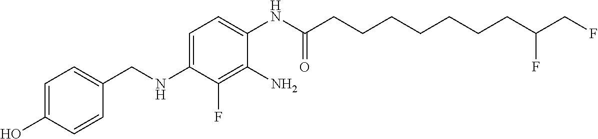 [SMILES: Nc1c(NC(=O)CCCCCCCC(F)CF)ccc(NCc2ccc(O)cc2)c1F] | N-(2-amino-3-fluoro-4-((4- hydroxybenzyl)amino)phenyl)- 9,10-difluorodecanamide (9R)-N-(2-amino-3-fluoro-4-((4- hydroxybenzyl)amino)phenyl)- 9,10-difluorodecanamide (9S)-N-(2-amino-3-fluoro-4-((4- hydroxybenzyl)amino)phenyl)- 9,10-difluorodecanamide |
| | racemic mixtures thereof |
| 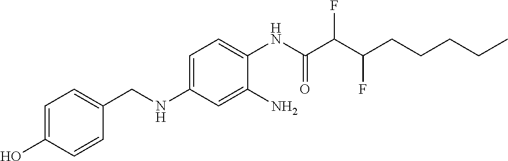 [SMILES: CCCCCC(F)C(F)C(=O)Nc1ccc(NCc2ccc(O)cc2)cc1N] | N-(2-amino-4-((4- hydroxybenzyl)amino)phenyl)- 2,3-difluorooctanamide (2R,3S)-N-(2-amino-4-((4- hydroxybenzyl)amino)phenyl)- 2,3-difluorooctanamide (2S,3R)-N-(2-amino-4-((4- hydroxybenzyl)amino)phenyl)- 2,3-difluorooctanamide (2R,3R)-N-(2-amino-4-((4- |
| | hydroxybenzyl)amino)phenyl)- |
| | 2,3-difluorooctanamide |
| | (2S,3S)-N-(2-amino-4-((4- |
| | hydroxybenzyl)amino)phenyl)- |
| | 2,3-difluorooctanamide |
| | racemic mixtures thereof |
| 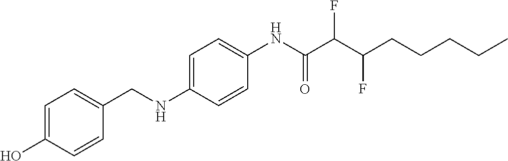 [SMILES: CCCCCC(F)C(F)C(=O)Nc1ccc(NCc2ccc(O)cc2)cc1] | 2,3-difluoro-N-(4-((4- hydroxybenzyl)amino)phenyl) octanamide (2R,3S)-2,3-difluoro-N-(4-((4- hydroxybenzyl)amino)phenyl) octanamide (2S,3R)-2,3-difluoro-N-(4-((4- hydroxybenzyl)amino)phenyl) octanamide (2R,3R)-2,3-difluoro-N-(4-((4- |
| | hydroxybenzyl)amino)phenyl) |
| | octanamide |
| | (2S,3S)-2,3-difluoro-N-(4-((4- |
| | hydroxybenzyl)amino)phenyl) |
| | octanamide |
| | racemic mixtures thereof |
| 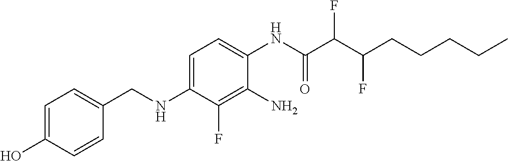 [SMILES: CCCCCC(F)C(F)C(=O)Nc1ccc(NCc2ccc(O)cc2)c(F)c1N] | N-(2-amino-3-fluoro-4-((4- hydroxybenzyl)amino)phenyl)- 2,3-difluorooctanamide (2R,3S)-N-(2-amino-3-fluoro-4- ((4- hydroxybenzyl)amino)phenyl)- 2,3-difluorooctanamide (2S,3R)-N-(2-amino-3-fluoro-4- ((4- hydroxybenzyl)amino)phenyl)- |
| | 2,3-difluorooctanamide |
| | (2R,3R)-N-(2-amino-3-fluoro-4- |
| | ((4- |
| | hydroxybenzyl)amino)phenyl)- |
| | 2,3-difluorooctanamide |
| | (2S,3S)-N-(2-amino-3-fluoro-4- |
| | ((4- |
| | hydroxybenzyl)amino)phenyl)- |
| | 2,3-difluorooctanamide |
| | racemic mixtures thereof |
| 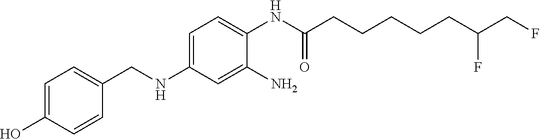 [SMILES: Nc1cc(NCc2ccc(O)cc2)ccc1NC(=O)CCCCCC(F)CF] | N-(2-amino-4-((4- hydroxybenzyl)amino)phenyl)- 7,8-difluorooctanamide (7S)-N-(2-amino-4-((4- hydroxybenzyl)amino)phenyl)- 7,8-difluorooctanamide (7R)-N-(2-amino-4-((4- hydroxybenzyl)amino)phenyl)- 7,8-difluorooctanamide |
| | racemic mixtures thereof |
| 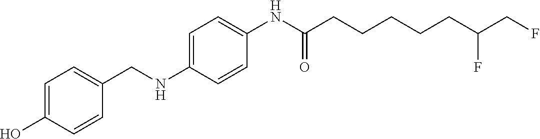 [SMILES: O=C(CCCCCC(F)CF)Nc1ccc(NCc2ccc(O)cc2)cc1] | 7,8-difluoro-N-(4-((4- hydroxybenzyl)amino)phenyl) octanamide (7R)-7,8-difluoro-N-(4-((4- hydroxybenzyl)amino)phenyl) octanamide (7S)-7,8-difluoro-N-(4-((4- hydroxybenzyl)amino)phenyl) octanamide |
| | racemic mixtures thereof |
| 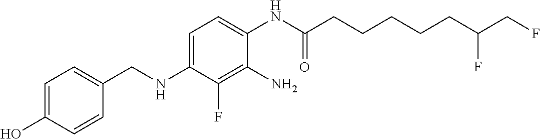 [SMILES: Nc1c(NC(=O)CCCCCC(F)CF)ccc(NCc2ccc(O)cc2)c1F] | N-(2-amino-3-fluoro-4-((4- hydroxybenzyl)amino)phenyl)- 7,8-difluorooctanamide (7R)-N-(2-amino-3-fluoro-4-((4- hydroxybenzyl)amino)phenyl)- 7,8-difluorooctanamide (7S)-N-(2-amino-3-fluoro-4-((4- hydroxybenzyl)amino)phenyl)- 7,8-difluorooctanamide |
| | racemic mixtures thereof |
| 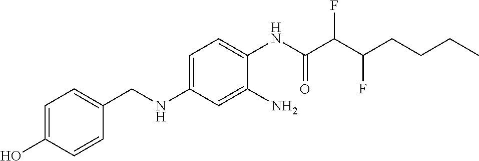 [SMILES: CCCCC(F)C(F)C(=O)Nc1ccc(NCc2ccc(O)cc2)cc1N] | N-(2-amino-4-((4- hydroxybenzyl)amino)phenyl)- 2,3-difluoroheptanamide (2R,3S)-N-(2-amino-4-((4- hydroxybenzyl)amino)phenyl)- 2,3-difluoroheptanamide (2S,3R)-N-(2-amino-4-((4- hydroxybenzyl)amino)phenyl)- 2,3-difluoroheptanamide (2R,3R)-N-(2-amino-4-((4- |
| | hydroxybenzyl)amino)phenyl)- |
| | 2,3-difluoroheptanamide |
| | (2S,3S)-N-(2-amino-4-((4- |
| | hydroxybenzyl)amino)phenyl)- |
| | 2,3-difluoroheptanamide |
| | racemic mixtures thereof |
| 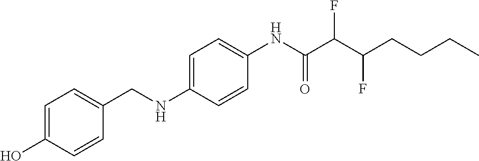 [SMILES: CCCCC(F)C(F)C(=O)Nc1ccc(NCc2ccc(O)cc2)cc1] | 2,3-difluoro-N-(4-((4- hydroxybenzyl)amino)phenyl) heptanamide (2R,3S)-2,3-difluoro-N-(4-((4- hydroxybenzyl)amino)phenyl) heptanamide (2S,3R)-2,3-difluoro-N-(4-((4- hydroxybenzyl)amino)phenyl) heptanamide (2R,3R)-2,3-difluoro-N-(4-((4- |
| | hydroxybenzyl)amino)phenyl) |
| | heptanamide |
| | (2S,3S)-2,3-difluoro-N-(4-((4- |
| | hydroxybenzyl)amino)phenyl) |
| | heptanamide |
| | racemic mixtures thereof |
| 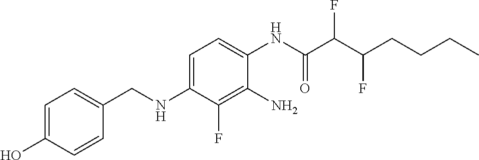 [SMILES: CCCCC(F)C(F)C(=O)Nc1ccc(NCc2ccc(O)cc2)c(F)c1N] | N-(2-amino-3-fluoro-4-((4- hydroxybenzyl)amino)phenyl)- 2,3-difluoroheptanamide (2R,3S)-N-(2-amino-3-fluoro-4- ((4- hydroxybenzyl)amino)phenyl)- 2,3-difluoroheptanamide (2S,3R)-N-(2-amino-3-fluoro-4- ((4- hydroxybenzyl)amino)phenyl)- |
| | 2,3-difluoroheptanamide |
| | (2R,3R)-N-(2-amino-3-fluoro-4- |
| | ((4- |
| | hydroxybenzyl)amino)phenyl)- |
| | 2,3-difluoroheptanamide |
| | (2S,3S)-N-(2-amino-3-fluoro-4- |
| | ((4- |
| | hydroxybenzyl)amino)phenyl)- |
| | 2,3-difluoroheptanamide |
| | racemic mixtures thereof |
| 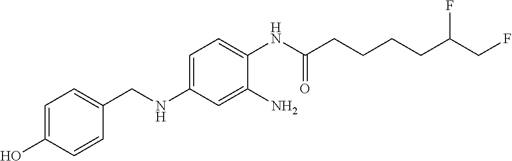 [SMILES: Nc1cc(NCc2ccc(O)cc2)ccc1NC(=O)CCCCC(F)CF] | N-(2-amino-4-((4- hydroxybenzyl)amino)phenyl)- 6,7-difluoroheptanamide (6R)-N-(2-amino-4-((4- hydroxybenzyl)amino)phenyl)- 6,7-difluoroheptanamide (6S)-N-(2-amino-4-((4- hydroxybenzyl)amino)phenyl)- 6,7-difluoroheptanamide racemic mixtures thereof |
| 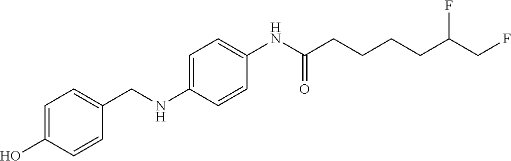 [SMILES: O=C(CCCCC(F)CF)Nc1ccc(NCc2ccc(O)cc2)cc1] | 6,7-difluoro-N-(4-((4- hydroxybenzyl)amino)phenyl) heptanamide (6R)-6,7-difluoro-N-(4-((4- hydroxybenzyl)amino)phenyl) heptanamide (6S)-6,7-difluoro-N-(4-((4- hydroxybenzyl)amino)phenyl) heptanamide racemic mixtures thereof |
| 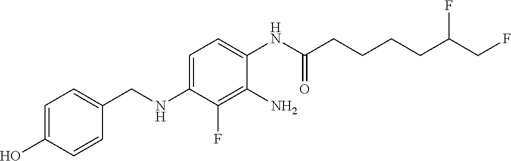 [SMILES: Nc1c(NC(=O)CCCCC(F)CF)ccc(NCc2ccc(O)cc2)c1F] | N-(2-amino-3-fluoro-4-((4- hydroxybenzyl)amino)phenyl)- 6,7-difluoroheptanamide (6R)-N-(2-amino-3-fluoro-4-((4- hydroxybenzyl)amino)phenyl)- 6,7-difluoroheptanamide (6S)-N-(2-amino-3-fluoro-4-((4- hydroxybenzyl)amino)phenyl)- 6,7-difluoroheptanamide racemic mixtures thereof |
| 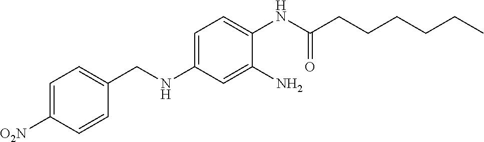 [SMILES: CCCCCCC(=O)Nc1ccc(NCc2ccc([N+](=O)[O-])cc2)cc1N] | N-(2-amino-4-((4- nitrobenzyl)amino)phenyl) heptanamide |
| 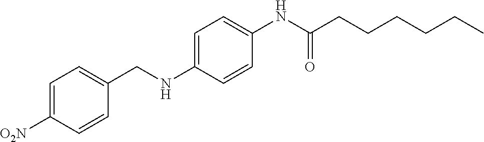 [SMILES: CCCCCCC(=O)Nc1ccc(NCc2ccc([N+](=O)[O-])cc2)cc1] | N-(4-((4- nitrobenzyl)amino)phenyl) heptanamide |
| 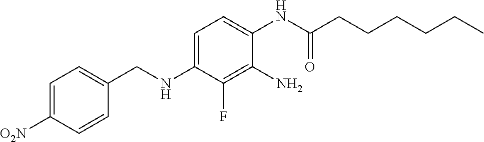 [SMILES: CCCCCCC(=O)Nc1ccc(NCc2ccc([N+](=O)[O-])cc2)c(F)c1N] | N-(2-amino-3-fluoro-4-((4- nitrobenzyl)amino)phenyl) heptanamide |
| 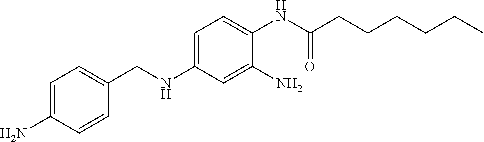 [SMILES: CCCCCCC(=O)Nc1ccc(NCc2ccc(N)cc2)cc1N] | N-(2-amino-4-((4- aminobenzyl)amino)phenyl) heptanamide |
| 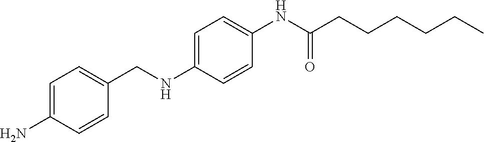 [SMILES: CCCCCCC(=O)Nc1ccc(NCc2ccc(N)cc2)cc1] | N-(4-((4- aminobenzyl)amino)phenyl) heptanamide |
| 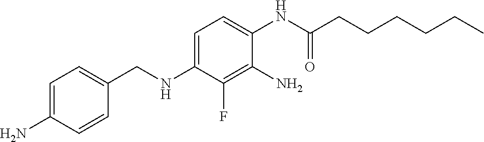 [SMILES: CCCCCCC(=O)Nc1ccc(NCc2ccc(N)cc2)c(F)c1N] | N-(2-amino-3-fluoro-4-((4- aminobenzyl)amino)phenyl) heptanamide |
| 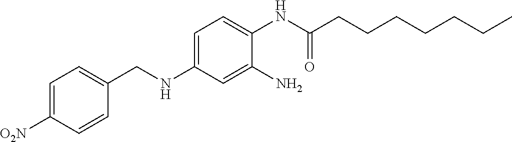 [SMILES: CCCCCCCC(=O)Nc1ccc(NCc2ccc([N+](=O)[O-])cc2)cc1N] | N-(2-amino-4-((4- nitrobenzyl)amino)phenyl) octanamide |
| 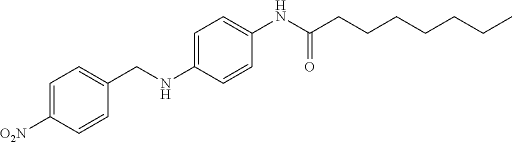 [SMILES: CCCCCCCC(=O)Nc1ccc(NCc2ccc([N+](=O)[O-])cc2)cc1] | N-(4-((4- nitrobenzyl)amino)phenyl) octanamide |
| 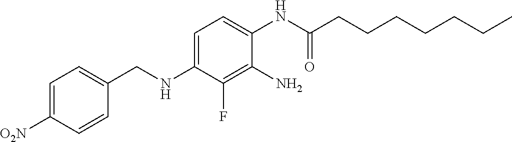 [SMILES: CCCCCCCC(=O)Nc1ccc(NCc2ccc([N+](=O)[O-])cc2)c(F)c1N] | N-(2-amino-3-fluoro-4-((4- nitrobenzyl)amino)phenyl) octanamide |
| 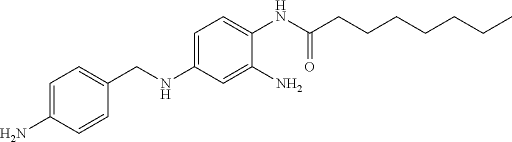 [SMILES: CCCCCCCC(=O)Nc1ccc(NCc2ccc(N)cc2)cc1N] | N-(2-amino-4-((4- aminobenzyl)amino)phenyl) octanamide |
| 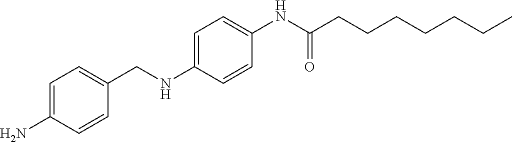 [SMILES: CCCCCCCC(=O)Nc1ccc(NCc2ccc(N)cc2)cc1] | N-(4-((4- aminobenzyl)amino)phenyl) octanamide |
| 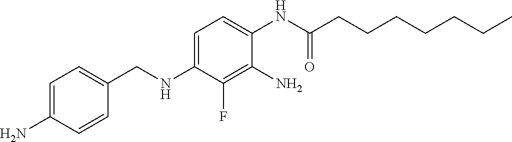 [SMILES: CCCCCCCC(=O)Nc1ccc(NCc2ccc(N)cc2)c(F)c1N] | N-(2-amino-3-fluoro-4-((4- aminobenzyl)amino)phenyl) octanamide |
| 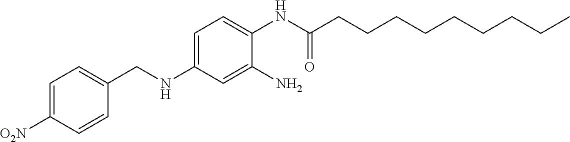 [SMILES: CCCCCCCCCC(=O)Nc1ccc(NCc2ccc([N+](=O)[O-])cc2)cc1N] | N-(2-amino-4-((4- nitrobenzyl)amino)phenyl) decanamide |
| 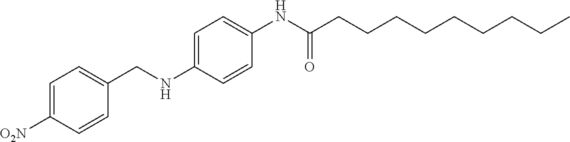 [SMILES: CCCCCCCCCC(=O)Nc1ccc(NCc2ccc([N+](=O)[O-])cc2)cc1] | N-(4-((4- nitrobenzyl)amino)phenyl) decanamide |
| 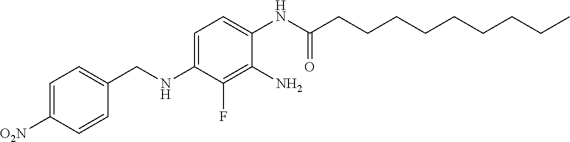 [SMILES: CCCCCCCCCC(=O)Nc1ccc(NCc2ccc([N+](=O)[O-])cc2)c(F)c1N] | N-(2-amino-3-fluoro-4-((4- nitrobenzyl)amino)phenyl) decanamide |
| 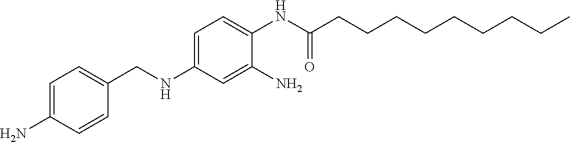 [SMILES: CCCCCCCCCC(=O)Nc1ccc(NCc2ccc(N)cc2)cc1N] | N-(2-amino-4-((4- aminobenzyl)amino)phenyl) decanamide |
| 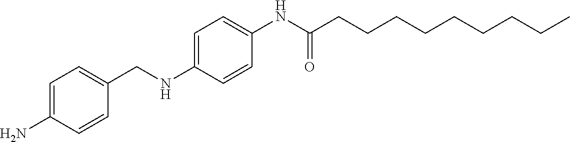 [SMILES: CCCCCCCCCC(=O)Nc1ccc(NCc2ccc(N)cc2)cc1] | N-(4-((4- aminobenzyl)amino)phenyl) decanamide |
| 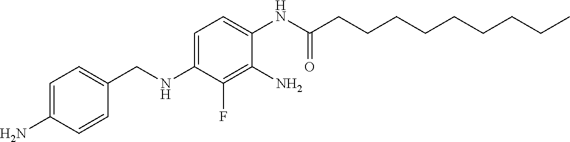 [SMILES: CCCCCCCCCC(=O)Nc1ccc(NCc2ccc(N)cc2)c(F)c1N] | N-(2-amino-3-fluoro-4-((4- aminobenzyl)amino)phenyl) decanamide |
| 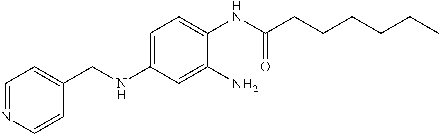 [SMILES: CCCCCCC(=O)Nc1ccc(NCc2ccncc2)cc1N] | N-(2-amino-4-((pyridin-4- ylmethyl)amino)phenyl) heptanamide |
| 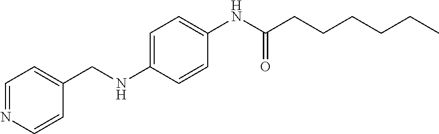 [SMILES: CCCCCCC(=O)Nc1ccc(NCc2ccncc2)cc1] | N-(4-((pyridin-4- ylmethyl)amino)phenyl) heptanamide |
| 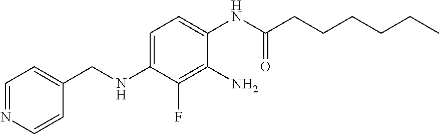 [SMILES: CCCCCCC(=O)Nc1ccc(NCc2ccncc2)c(F)c1N] | N-(2-amino-3-fluoro-4- ((pyridin-4- ylmethyl)amino)phenyl) heptanamide |
| 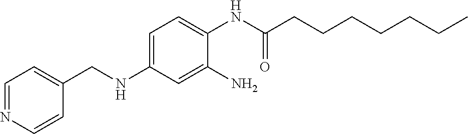 [SMILES: CCCCCCCC(=O)Nc1ccc(NCc2ccncc2)cc1N] | N-(2-amino-4-((pyridin-4- ylmethyl)amino)phenyl) octanamide |
| 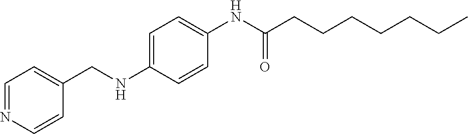 [SMILES: CCCCCCCC(=O)Nc1ccc(NCc2ccncc2)cc1] | N-(4-((pyridin-4- ylmethyl)amino)phenyl) octanamide |
| 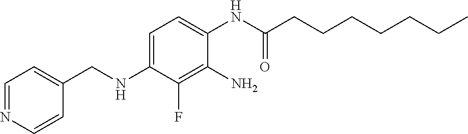 [SMILES: CCCCCCCC(=O)Nc1ccc(NCc2ccncc2)c(F)c1N] | N-(2-amino-3-fluoro-4- ((pyridin-4- ylmethyl)amino)phenyl) octanamide |
| 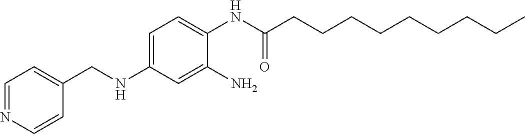 [SMILES: CCCCCCCCCC(=O)Nc1ccc(NCc2ccncc2)cc1N] | N-(2-amino-4-((pyridin-4- ylmethyl)amino)phenyl) decanamide |
| 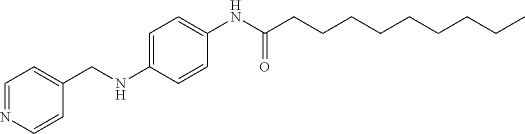 [SMILES: CCCCCCCCCC(=O)Nc1ccc(NCc2ccncc2)cc1] | N-(4-((pyridin-4- ylmethyl)amino)phenyl) decanamide |
| 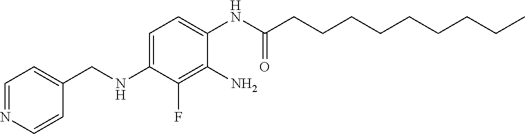 [SMILES: CCCCCCCCCC(=O)Nc1ccc(NCc2ccncc2)c(F)c1N] | N-(2-amino-3-fluoro-4- ((pyridin-4- ylmethyl)amino)phenyl) decanamide |
| 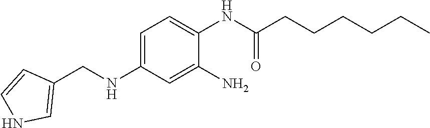 [SMILES: CCCCCCC(=O)Nc1ccc(NCc2cc[nH]c2)cc1N] | N-(4-(((1H-pyrrol-3- yl)methyl)amino)-2- aminophenyl)heptanamide |
| 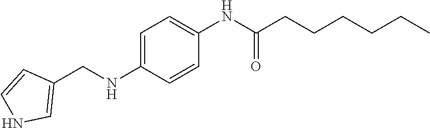 [SMILES: CCCCCCC(=O)Nc1ccc(NCc2cc[nH]c2)cc1] | N-(4-(((1H-pyrrol-3- yl)methyl)amino)phenyl) heptanamide |
| 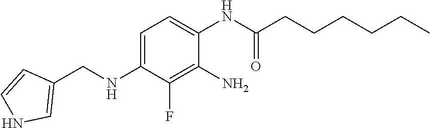 [SMILES: CCCCCCC(=O)Nc1ccc(NCc2cc[nH]c2)c(F)c1N] | N-(4-(((1H-pyrrol-3- yl)methyl)amino)-2-amino-3- fluorophenyl)heptanamide |
| 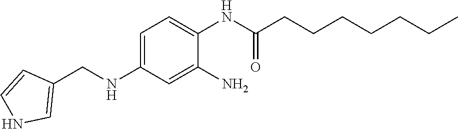 [SMILES: CCCCCCCC(=O)Nc1ccc(NCc2cc[nH]c2)cc1N] | N-(4-(((1H-pyrrol-3- yl)methyl)amino)-2- aminophenyl)octanamide |
| 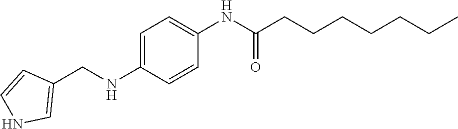 [SMILES: CCCCCCCC(=O)Nc1ccc(NCc2cc[nH]c2)cc1] | N-(4-(((1H-pyrrol-3- yl)methyl)amino)phenyl) octanamide |
| 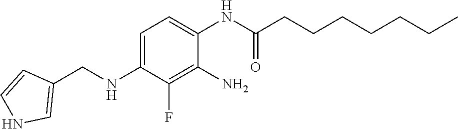 [SMILES: CCCCCCCC(=O)Nc1ccc(NCc2cc[nH]c2)c(F)c1N] | N-(4-(((1H-pyrrol-3- yl)methyl)amino)-2-amino-3- fluorophenyl)octanamide |
| 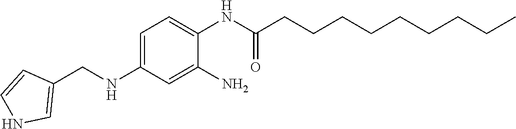 [SMILES: CCCCCCCCCC(=O)Nc1ccc(NCc2cc[nH]c2)cc1N] | N-(4-(((1H-pyrrol-3- yl)methyl)amino)-2- aminophenyl)decanamide |
| 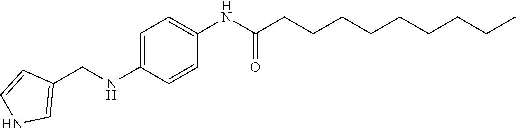 [SMILES: CCCCCCCCCC(=O)Nc1ccc(NCc2cc[nH]c2)cc1] | N-(4-(((1H-pyrrol-3- yl)methyl)amino)phenyl) decanamide |
| 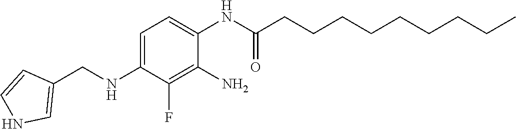 [SMILES: CCCCCCCCCC(=O)Nc1ccc(NCc2cc[nH]c2)c(F)c1N] | N-(4-(((1H-pyrrol-3- yl)methyl)amino)-2-amino-3- fluorophenyl)decanamide |
| 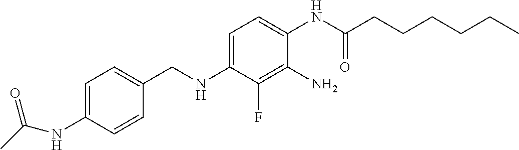 [SMILES: CCCCCCC(=O)Nc1ccc(NCc2ccc(NC(C)=O)cc2)c(F)c1N] | N-(4-((4- acetamidobenzyl)amino)-2- amino-3- fluorophenyl)heptanamide |
| 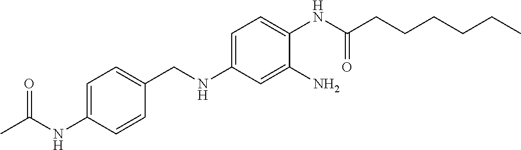 [SMILES: CCCCCCC(=O)Nc1ccc(NCc2ccc(NC(C)=O)cc2)cc1N] | N-(4-((4- acetamidobenzyl)amino)-2- aminophenyl)heptanamide |
| 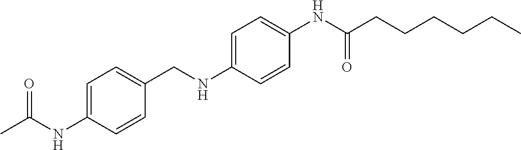 [SMILES: CCCCCCC(=O)Nc1ccc(NCc2ccc(NC(C)=O)cc2)cc1] | N-(4-((4- acetamidobenzyl)amino)phenyl) heptanamide |
| 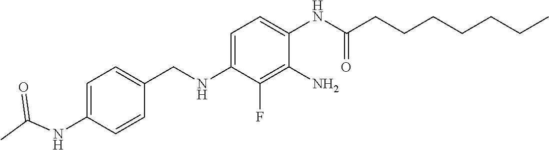 [SMILES: CCCCCCCC(=O)Nc1ccc(NCc2ccc(NC(C)=O)cc2)c(F)c1N] | N-(4-((4- acetamidobenzyl)amino)-2- amino-3- fluorophenyl)octanamide |
| 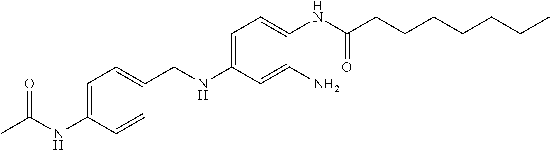 [SMILES: C=C/C(=C\C=C\CNC(/C=C/N)=C/C=C/NC(=O)CCCCCCC)NC(C)=O] | N-(4-((4- acetamidobenzyl)amino)-2- aminophenyl)octanamide |
| 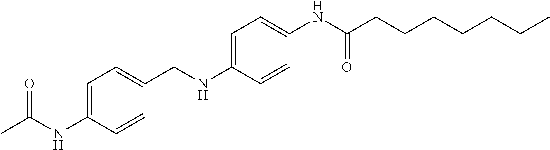 [SMILES: C=C/C(=C\C=C\NC(=O)CCCCCCC)NC/C=C/C=C(\C=C)NC(C)=O] | N-(4-((4- acetamidobenzyl)amino)phenyl) octanamide |
| 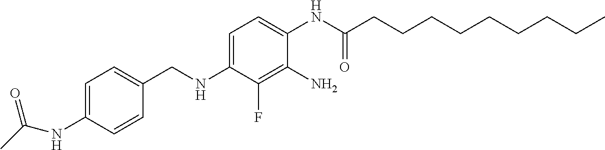 [SMILES: CCCCCCCCCC(=O)Nc1ccc(NCc2ccc(NC(C)=O)cc2)c(F)c1N] | N-(4-((4- acetamidobenzyl)amino)-2- amino-3- fluorophenyl)decanamide |
| 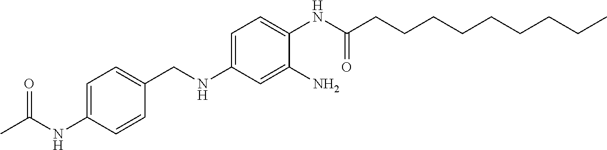 [SMILES: CCCCCCCCCC(=O)Nc1ccc(NCc2ccc(NC(C)=O)cc2)cc1N] | N-(4-((4- acetamidobenzyl)amino)-2- aminophenyl)decanamide |
| 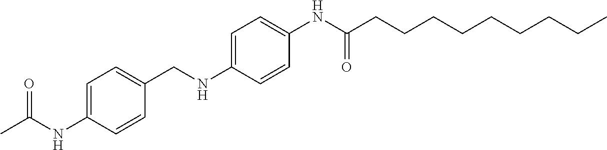 [SMILES: CCCCCCCCCC(=O)Nc1ccc(NCc2ccc(NC(C)=O)cc2)cc1] | N-(4-((4- acetamidobenzyl)amino)phenyl) decanamide |
| 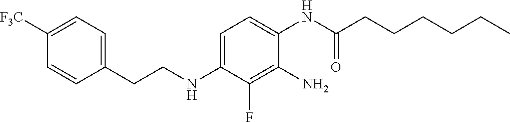 [SMILES: CCCCCCC(=O)Nc1ccc(NCCc2ccc(C(F)(F)F)cc2)c(F)c1N] | N-(2-amino-3-fluoro-4-((4- (trifluoromethyl)phenethyl) amino)phenyl)heptanamide |
| 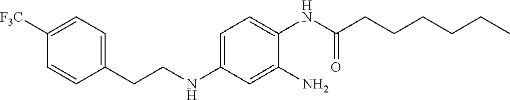 [SMILES: CCCCCCC(=O)Nc1ccc(NCCc2ccc(C(F)(F)F)cc2)cc1N] | N-(2-amino-4-((4- (trifluoromethyl)phenethyl) amino)phenyl)heptanamide |
| 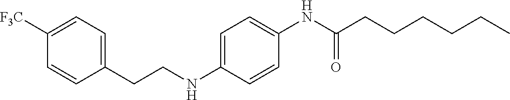 [SMILES: CCCCCCC(=O)Nc1ccc(NCCc2ccc(C(F)(F)F)cc2)cc1] | N-(4-((4- (trifluoromethyl)phenethyl) amino)phenyl)heptanamide |
| 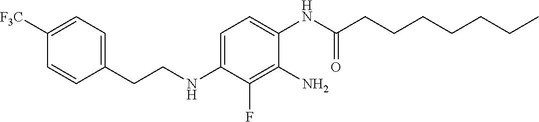 [SMILES: CCCCCCCC(=O)Nc1ccc(NCCc2ccc(C(F)(F)F)cc2)c(F)c1N] | N-(2-amino-3-fluoro-4-((4- (trifluoromethyl)phenethyl) amino)phenyl)octanamide |
| 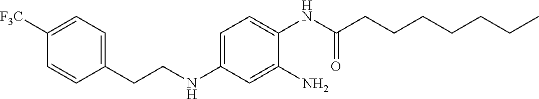 [SMILES: CCCCCCCC(=O)Nc1ccc(NCCc2ccc(C(F)(F)F)cc2)cc1N] | N-(2-amino-4-((4- (trifluoromethyl)phenethyl) amino)pheny)loctanamide |
| 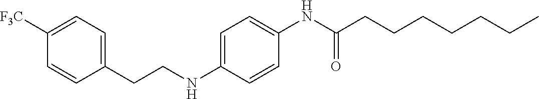 [SMILES: CCCCCCCC(=O)Nc1ccc(NCCc2ccc(C(F)(F)F)cc2)cc1] | N-(4-((4- (trifluoromethyl)phenethyl) amino)phenyl)octanamide |
| 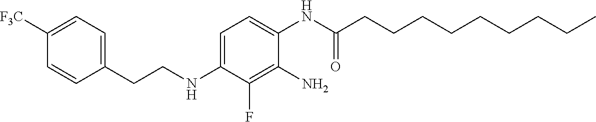 [SMILES: CCCCCCCCCC(=O)Nc1ccc(NCCc2ccc(C(F)(F)F)cc2)c(F)c1N] | N-(2-amino-3-fluoro-4-((4- (trifluoromethyl)phenethyl) amino)phenyl)decanamide |
| 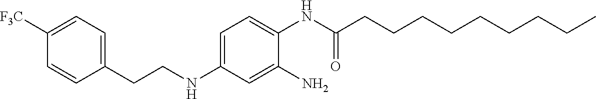 [SMILES: CCCCCCCCCC(=O)Nc1ccc(NCCc2ccc(C(F)(F)F)cc2)cc1N] | N-(2-amino-4-((4- (trifluoromethyl)phenethyl) amino)phenyl)decanamide |
| 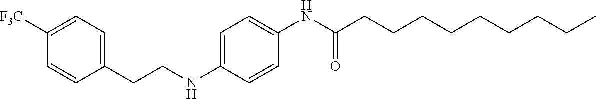 [SMILES: CCCCCCCCCC(=O)Nc1ccc(NCCc2ccc(C(F)(F)F)cc2)cc1] | N-(4-((4- (trifluoromethyl)phenethyl) amino)phenyl)decanamide |
| 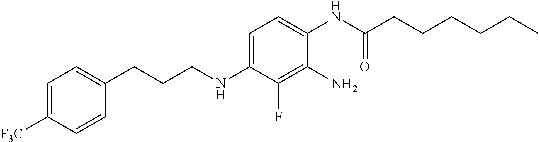 [SMILES: CCCCCCC(=O)Nc1ccc(NCCCc2ccc(C(F)(F)F)cc2)c(F)c1N] | N-(2-amino-3-fluoro-4-((3-(4- (trifluoromethyl)phenyl)propyl) amino)phenyl)heptanamide |
| 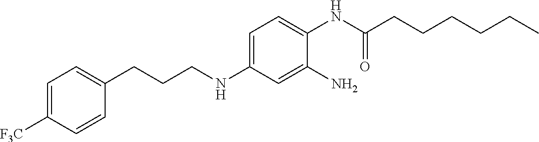 [SMILES: CCCCCCC(=O)Nc1ccc(NCCCc2ccc(C(F)(F)F)cc2)cc1N] | N-(2-amino-4-((3-(4- (trifluoromethyl)phenyl)propyl) amino)phenyl)heptanamide |
| 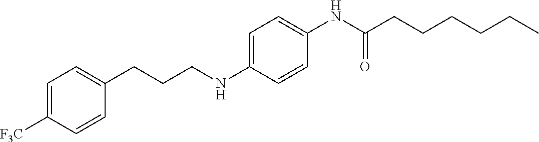 [SMILES: CCCCCCC(=O)Nc1ccc(NCCCc2ccc(C(F)(F)F)cc2)cc1] | N-(4-((3-(4- (trifluoromethyl)phenyl)propyl) amino)phenyl)heptanamide |
| 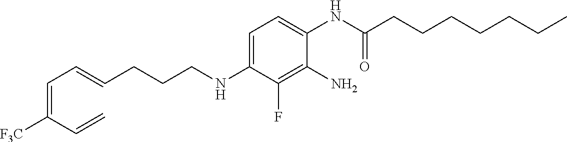 [SMILES: C=C/C(=C\C=C\CCCNc1ccc(NC(=O)CCCCCCC)c(N)c1F)C(F)(F)F] | N-(2-amino-3-fluoro-4-((3-(4- (trifluoromethyl)phenyl)propyl) amino)phenyl)octanamide |
| 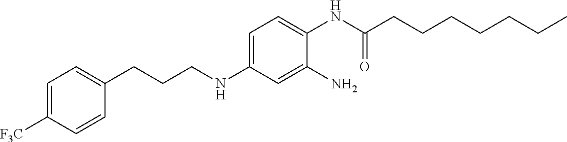 [SMILES: CCCCCCCC(=O)Nc1ccc(NCCCc2ccc(C(F)(F)F)cc2)cc1N] | N-(2-amino-4-((3-(4- (trifluoromethyl)phenyl)propyl) amino)phenyl)octanamide |
| 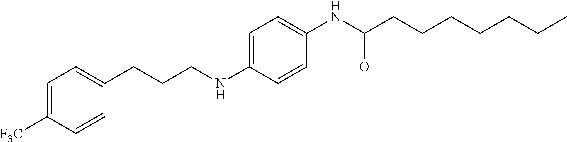 [SMILES: C=C/C(=C\C=C\CCCNc1ccc(NC(O)CCCCCCC)cc1)C(F)(F)F] | N-(4-((3-(4- (trifluoromethyl)phenyl)propyl) amino)phenyl)octanamide |
| 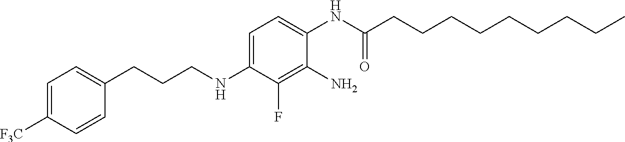 [SMILES: CCCCCCCCCC(=O)Nc1ccc(NCCCc2ccc(C(F)(F)F)cc2)c(F)c1N] | N-(2-amino-3-fluoro-4-((3-(4- (trifluoromethyl)phenyl)propyl) amino)phenyl)decanamide |
| 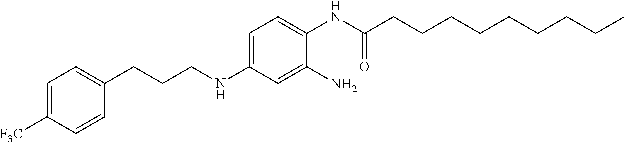 [SMILES: CCCCCCCCCC(=O)Nc1ccc(NCCCc2ccc(C(F)(F)F)cc2)cc1N] | N-(2-amino-4-((3-(4- (trifluoromethyl)phenyl)propyl) amino)phenyl)decanamide |
| 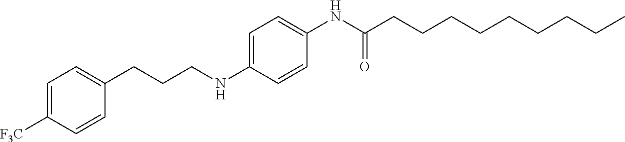 [SMILES: CCCCCCCCCC(=O)Nc1ccc(NCCCc2ccc(C(F)(F)F)cc2)cc1] | N-(4-((3-(4- (trifluoromethyl)phenyl)propyl) amino)phenyl)decanamide |
| 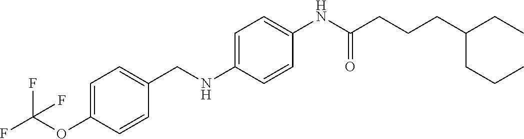 [SMILES: CCCC(CC)CCCC(=O)Nc1ccc(NCc2ccc(OC(F)(F)F)cc2)cc1] | 4-cyclohexyl-N-(4-((4- (trifluoromethoxy)benzyl)amino) phenyl)butanamide |
| 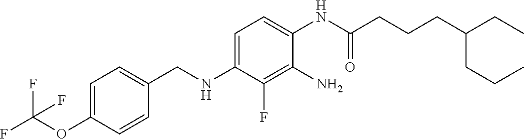 [SMILES: CCCC(CC)CCCC(=O)Nc1ccc(NCc2ccc(OC(F)(F)F)cc2)c(F)c1N] | N-(2-amino-3-fluoro-4-((4- (trifluoromethoxy)benzyl)amino) phenyl)-4- cyclohexylbutanamide |
| 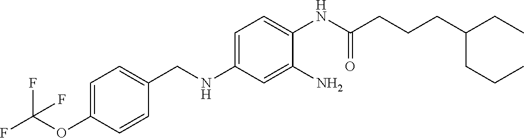 [SMILES: CCCC(CC)CCCC(=O)Nc1ccc(NCc2ccc(OC(F)(F)F)cc2)cc1N] | N-(2-amino-4-((4- (trifluoromethoxy)benzyl)amino) phenyl)-4- cyclohexylbutanamide |
| 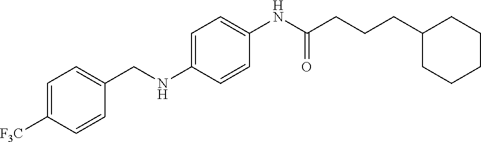 [SMILES: O=C(CCCC1CCCCC1)Nc1ccc(NCc2ccc(C(F)(F)F)cc2)cc1] | 4-cyclohexyl-N-(4-((4- (trifluoromethyl)benzyl)amino) phenyl)butanamide |
| 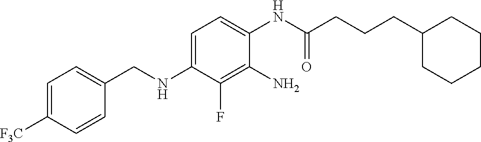 [SMILES: Nc1c(NC(=O)CCCC2CCCCC2)ccc(NCc2ccc(C(F)(F)F)cc2)c1F] | N-(2-amino-3-fluoro-4-((4- (trifluoromethyl)benzyl)amino) phenyl)-4-cyclohexylbutanamide |
| 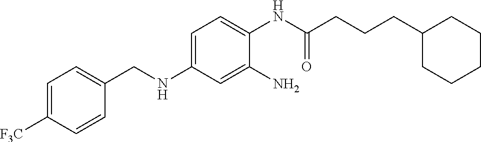 [SMILES: Nc1cc(NCc2ccc(C(F)(F)F)cc2)ccc1NC(=O)CCCC1CCCCC1] | N-(2-amino-4-((4- (trifluoromethyl)benzyl)amino) phenyl)-4-cyclohexylbutanamide |
| 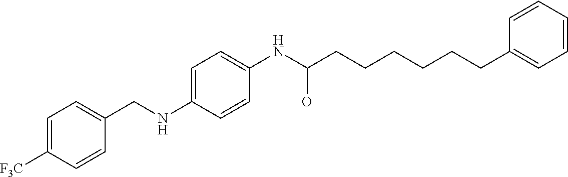 [SMILES: OC(CCCCCCc1ccccc1)Nc1ccc(NCc2ccc(C(F)(F)F)cc2)cc1] | 7-phenyl-N-(4-((4- (trifluoromethyl)benzyl)amino) phenyl)heptanamide |
| 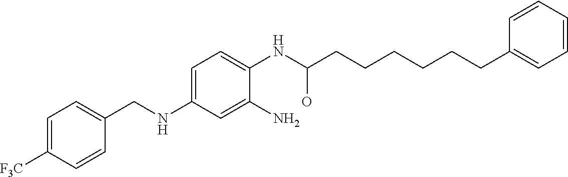 [SMILES: Nc1cc(NCc2ccc(C(F)(F)F)cc2)ccc1NC(O)CCCCCCc1ccccc1] | N-(2-amino-4-((4- (trifluoromethyl)benzyl)amino) phenyl)-7-phenylheptanamide |
| 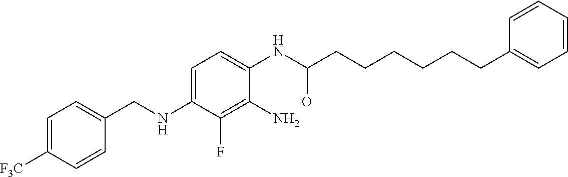 [SMILES: Nc1c(NC(O)CCCCCCc2ccccc2)ccc(NCc2ccc(C(F)(F)F)cc2)c1F] | N-(2-amino-3-fluoro-4-((4- (trifluoromethyl)benzyl)amino) phenyl)-7-phenylheptanamide |
| 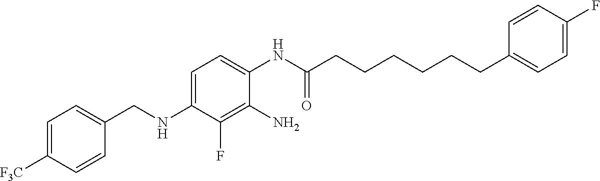 [SMILES: Nc1c(NC(=O)CCCCCCc2ccc(F)cc2)ccc(NCc2ccc(C(F)(F)F)cc2)c1F] | N-(2-amino-3-fluoro-4-((4- (trifluoromethyl)benzyl)amino) phenyl)-7-(4- fluorophenyl)heptanamide |
| 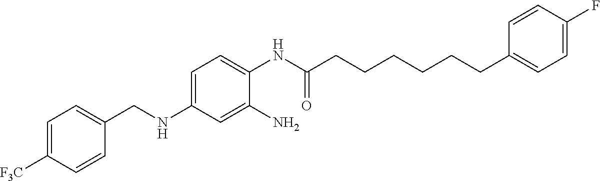 [SMILES: Nc1cc(NCc2ccc(C(F)(F)F)cc2)ccc1NC(=O)CCCCCCc1ccc(F)cc1] | N-(2-amino-4-((4- (trifluoromethyl)benzyl)amino) phenyl)-7-(4- fluorophenyl)heptanamide |
| 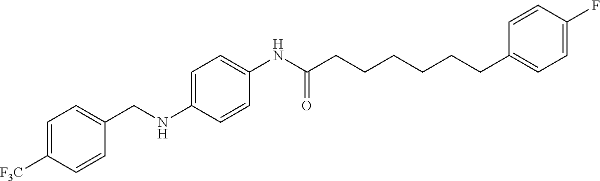 [SMILES: O=C(CCCCCCc1ccc(F)cc1)Nc1ccc(NCc2ccc(C(F)(F)F)cc2)cc1] | 7-(4-fluorophenyl)-N-(4-((4- (trifluoromethyl)benzyl)amino) phenyl)heptanamide |
| 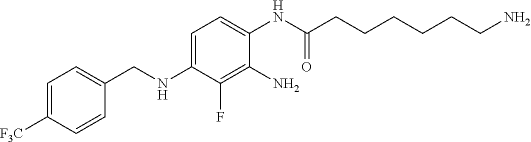 [SMILES: NCCCCCCC(=O)Nc1ccc(NCc2ccc(C(F)(F)F)cc2)c(F)c1N] | 7-amino-N-(2-amino-3-fluoro-4- ((4- (trifluoromethyl)benzyl)amino) phenyl)heptanamide |
| 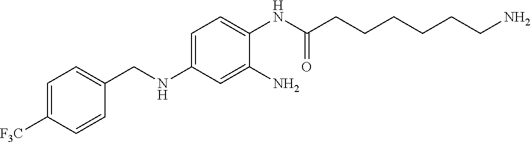 [SMILES: NCCCCCCC(=O)Nc1ccc(NCc2ccc(C(F)(F)F)cc2)cc1N] | 7-amino-N-(2-amino-4-((4- (trifluoromethyl)benzyl)amino) phenyl)heptanamide |
| 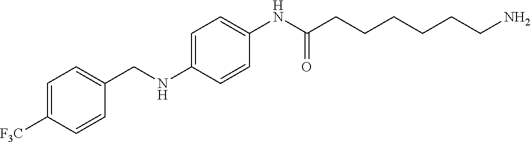 [SMILES: NCCCCCCC(=O)Nc1ccc(NCc2ccc(C(F)(F)F)cc2)cc1] | 7-amino-N-(4-((4- (trifluoromethyl)benzyl)amino) phenyl)heptanamide |
| 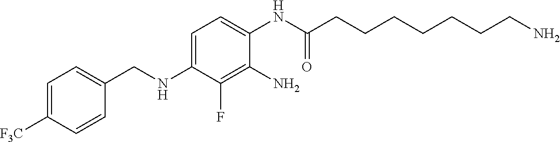 [SMILES: NCCCCCCCC(=O)Nc1ccc(NCc2ccc(C(F)(F)F)cc2)c(F)c1N] | 8-amino-N-(2-amino-3-fluoro-4- ((4- (trifluoromethyl)benzyl)amino) phenyl)octanamide |
| 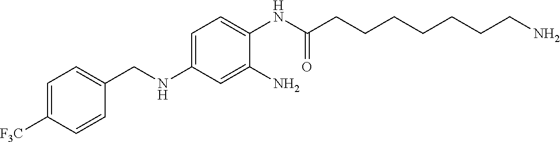 [SMILES: NCCCCCCCC(=O)Nc1ccc(NCc2ccc(C(F)(F)F)cc2)cc1N] | 8-amino-N-(2-amino-4-((4- (trifluoromethyl)benzyl)amino) phenyl)octanamide |
| 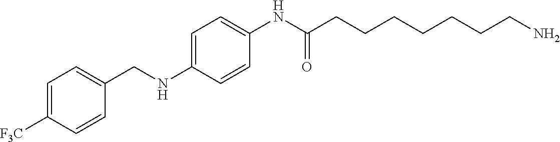 [SMILES: NCCCCCCCC(=O)Nc1ccc(NCc2ccc(C(F)(F)F)cc2)cc1] | 8-amino-N-(4-((4- (trifluoromethyl)benzyl)amino) phenyl)octanamide |
| 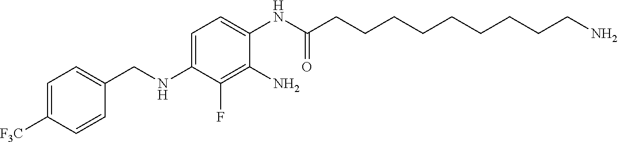 [SMILES: NCCCCCCCCCC(=O)Nc1ccc(NCc2ccc(C(F)(F)F)cc2)c(F)c1N] | 10-amino-N-(2-amino-3-fluoro- 4-((4- (trifluoromethyl)benzyl)amino) phenyl)decanamide |
| 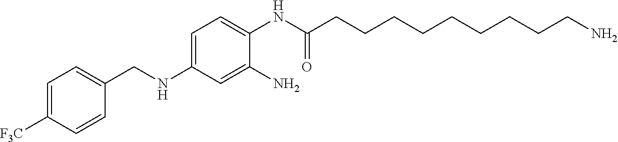 [SMILES: NCCCCCCCCCC(=O)Nc1ccc(NCc2ccc(C(F)(F)F)cc2)cc1N] | 10-amino-N-(2-amino-4-((4- (trifluoromethyl)benzyl)amino) phenyl)decanamide |
| 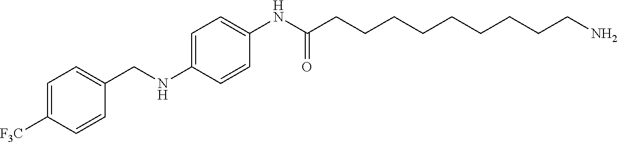 [SMILES: NCCCCCCCCCC(=O)Nc1ccc(NCc2ccc(C(F)(F)F)cc2)cc1] | 10-amino-N-(4-((4- (trifluoromethyl)benzyl)amino) phenyl)decanamide |
| 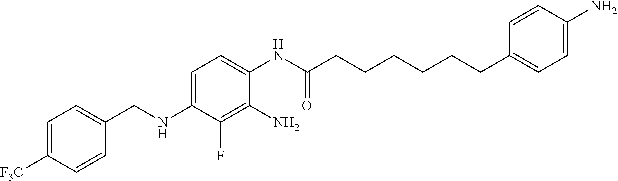 [SMILES: Nc1ccc(CCCCCCC(=O)Nc2ccc(NCc3ccc(C(F)(F)F)cc3)c(F)c2N)cc1] | N-(2-amino-3-fluoro-4-((4- (trifluoromethyl)benzyl)amino) phenyl)-7-(4- aminophenyl)heptanamide |
| 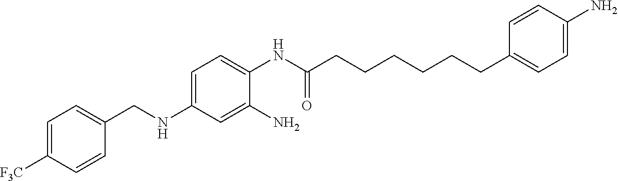 [SMILES: Nc1ccc(CCCCCCC(=O)Nc2ccc(NCc3ccc(C(F)(F)F)cc3)cc2N)cc1] | N-(2-amino-4-((4- (trifluoromethyl)benzyl)amino) phenyl)-7-(4- aminophenyl)heptanamide |
| 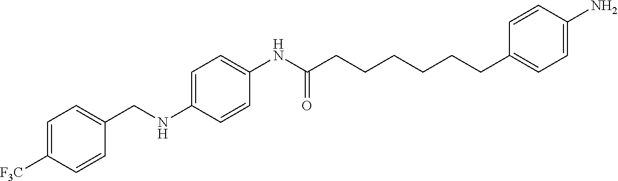 [SMILES: Nc1ccc(CCCCCCC(=O)Nc2ccc(NCc3ccc(C(F)(F)F)cc3)cc2)cc1] | 7-(4-aminophenyl)-N-(4-((4- (trifluoromethyl)benzyl)amino) phenyl)heptanamide |
| 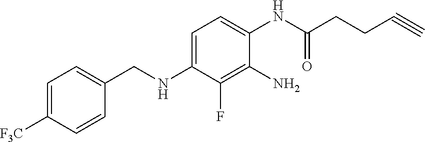 [SMILES: C#CCCC(=O)Nc1ccc(NCc2ccc(C(F)(F)F)cc2)c(F)c1N] | N-(2-amino-3-fluoro-4-((4- (trifluoromethyl)benzyl)amino) phenyl)pent-4-ynamide |
| 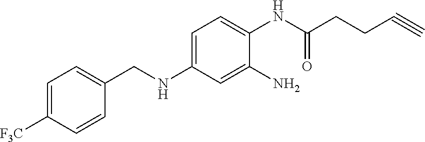 [SMILES: C#CCCC(=O)Nc1ccc(NCc2ccc(C(F)(F)F)cc2)cc1N] | N-(2-amino-4-((4- (trifluoromethyl)benzyl)amino) phenyl)pent-4-ynamide |
| 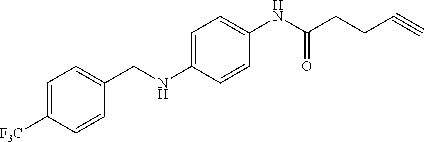 [SMILES: C#CCCC(=O)Nc1ccc(NCc2ccc(C(F)(F)F)cc2)cc1] | N-(4-((4- (trifluoromethyl)benzyl)amino) phenyl)pent-4-ynamide |
| 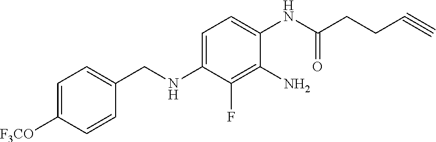 [SMILES: C#CCCC(=O)Nc1ccc(NCc2ccc(OC(F)(F)F)cc2)c(F)c1N] | N-(2-amino-3-fluoro-4-((4- (trifluoromethoxy)benzyl)amino) phenyl)pent-4-ynamide |
| 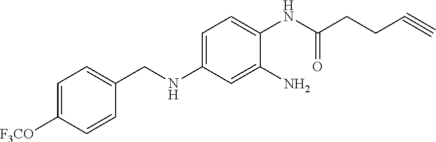 [SMILES: C#CCCC(=O)Nc1ccc(NCc2ccc(OC(F)(F)F)cc2)cc1N] | N-(2-amino-4-((4- (trifluoromethoxy)benzyl)amino) phenyl)pent-4-ynamide |
| 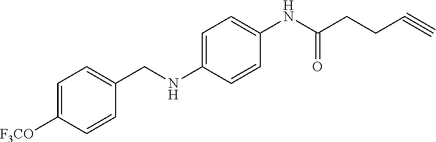 [SMILES: C#CCCC(=O)Nc1ccc(NCc2ccc(OC(F)(F)F)cc2)cc1] | N-(4-((4- (trifluoromethoxy)benzyl)amino) phenyl)pent-4-ynamide |
| 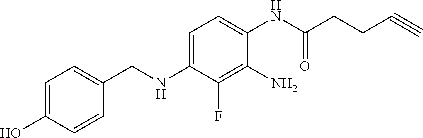 [SMILES: C#CCCC(=O)Nc1ccc(NCc2ccc(O)cc2)c(F)c1N] | N-(2-amino-3-fluoro-4-((4- hydroxybenzyl)amino)phenyl) pent-4-ynamide |
| 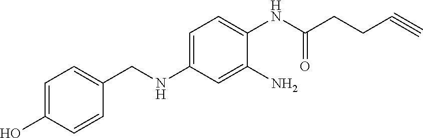 [SMILES: C#CCCC(=O)Nc1ccc(NCc2ccc(O)cc2)cc1N] | N-(2-amino-4-((4- hydroxybenzyl)amino)phenyl) pent-4-ynamide |
| 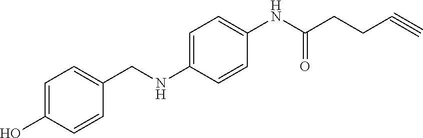 [SMILES: C#CCCC(=O)Nc1ccc(NCc2ccc(O)cc2)cc1] | N-(4-((4- hydroxybenzyl)amino)phenyl) pent-4-ynamide |
| 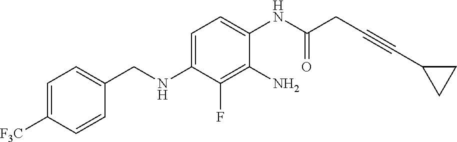 [SMILES: Nc1c(NC(=O)CC#CC2CC2)ccc(NCc2ccc(C(F)(F)F)cc2)c1F] | N-(2-amino-3-fluoro-4-((4- (trifluoromethyl)benzyl)amino) phenyl)-4-cyclopropylbut-3- ynamide |
| 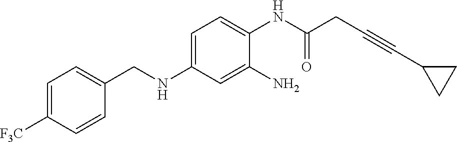 [SMILES: Nc1cc(NCc2ccc(C(F)(F)F)cc2)ccc1NC(=O)CC#CC1CC1] | N-(2-amino-4-((4- (trifluoromethyl)benzyl)amino) phenyl)-4-cyclopropylbut-3- ynamide |
| 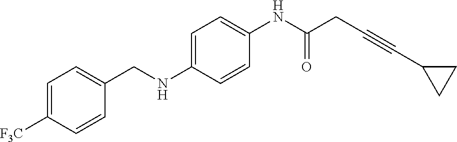 [SMILES: O=C(CC#CC1CC1)Nc1ccc(NCc2ccc(C(F)(F)F)cc2)cc1] | 4-cyclopropyl-N-(4-((4- (trifluoromethyl)benzyl)amino) phenyl)but-3-ynamide |
| 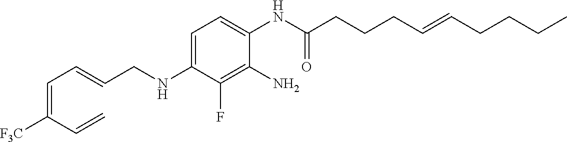 [SMILES: C=C/C(=C\C=C\CNc1ccc(NC(=O)CCC/C=C/CCCC)c(N)c1F)C(F)(F)F] | (E)-N-(2-amino-3-fluoro-4-((4- (trifluoromethyl)benzyl)amino) phenyl)dec-5-enamide |
| 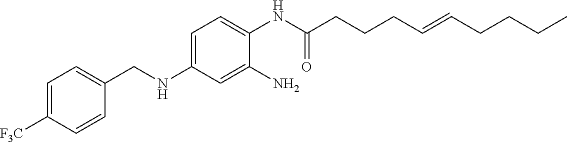 [SMILES: CCCC/C=C/CCCC(=O)Nc1ccc(NCc2ccc(C(F)(F)F)cc2)cc1N] | (E)-N-(2-amino-4-((4- (trifluoromethyl)benzyl)amino) phenyl)dec-5-enamide |
| 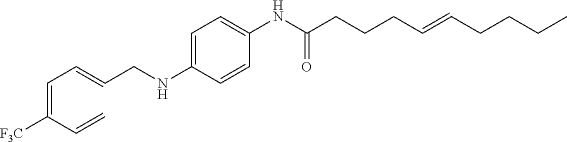 [SMILES: C=C/C(=C\C=C\CNc1ccc(NC(=O)CCC/C=C/CCCC)cc1)C(F)(F)F] | (E)-N-(4-((4- (trifluoromethyl)benzyl)amino) phenyl)dec-5-enamide |
| 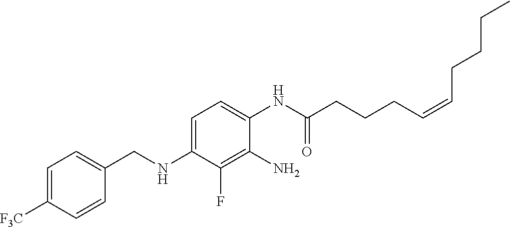 [SMILES: CCCC/C=C\CCCC(=O)Nc1ccc(NCc2ccc(C(F)(F)F)cc2)c(F)c1N] | (Z)-N-(2-amino-3-fluoro-4-((4- (trifluoromethyl)benzyl)amino) phenyl)dec-5-enamide |
| 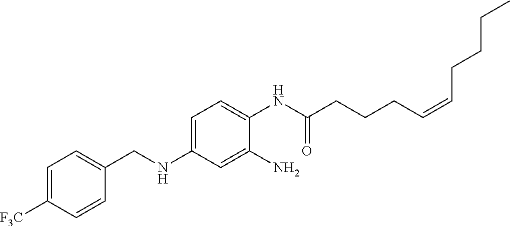 [SMILES: CCCC/C=C\CCCC(=O)Nc1ccc(NCc2ccc(C(F)(F)F)cc2)cc1N] | (Z)-N-(2-amino-4-((4- (trifluoromethyl)benzyl)amino) phenyl)dec-5-enamide |
| 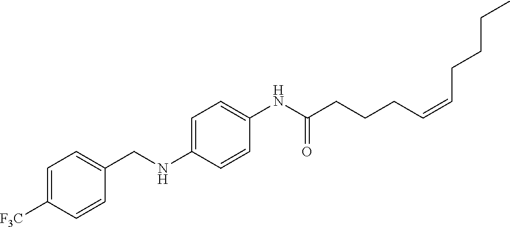 [SMILES: CCCC/C=C\CCCC(=O)Nc1ccc(NCc2ccc(C(F)(F)F)cc2)cc1] | (Z)-N-(4-((4- (trifluoromethyl)benzyl)amino) phenyl)dec-5-enamide |
| 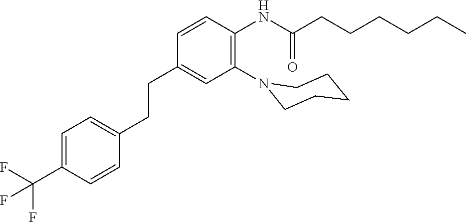 [SMILES: CCCCCCC(=O)Nc1ccc(CCc2ccc(C(F)(F)F)cc2)cc1N1CCCCC1] | N-(2-(piperidin-1-yl)-4-(4- (trifluoromethyl)phenethyl) phenyl)heptanamide |
| 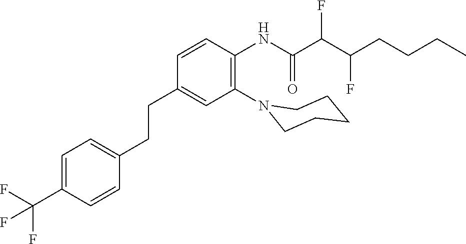 [SMILES: CCCCC(F)C(F)C(=O)Nc1ccc(CCc2ccc(C(F)(F)F)cc2)cc1N1CCCCC1] | 2,3-difluoro-N-(2-(piperidin-1- yl)-4-(4- (trifluoromethyl)phenethyl) phenyl)heptanamide (2R,3S)-2,3-difluoro-N-(2- (piperidin-1-yl)-4-(4- (trifluoromethyl)phenethyl) phenyl)heptanamide (2S,3R)-2,3-difluoro-N-(2- (piperidin-1-yl)-4-(4- (trifluoromethyl)phenethyl) phenyl)heptanamide (2R,3R)-2,3-difluoro-N-(2- (piperidin-1-yl)-4-(4- (trifluoromethyl)phenethyl) |
| | phenyl)heptanamide |
| | (2S,3S)-2,3-difluoro-N-(2- |
| | (piperidin-1-yl)-4-(4- |
| | (trifluoromethyl)phenethyl) |
| | phenyl)heptanamide |
| 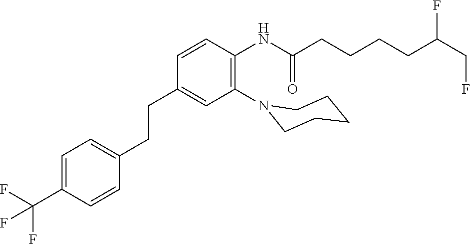 [SMILES: O=C(CCCCC(F)CF)Nc1ccc(CCc2ccc(C(F)(F)F)cc2)cc1N1CCCCC1] | 6,7-difluoro-N-(2-(piperidin-1- yl)-4-(4- (trifluoromethyl)phenethyl) phenyl)heptanamide (6R)-6,7-difluoro-N-(2- (piperidin-1-yl)-4-(4- (trifluoromethyl)phenethyl) phenyl)heptanamide (6S)-6,7-difluoro-N-(2- (piperidin-1-yl)-4-(4- (trifluoromethyl)phenethyl) phenyl)heptanamide |
| 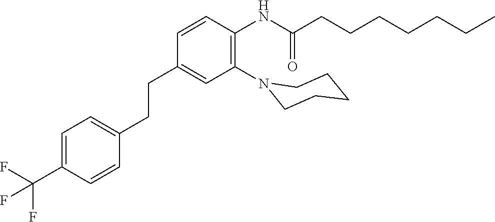 [SMILES: CCCCCCCC(=O)Nc1ccc(CCc2ccc(C(F)(F)F)cc2)cc1N1CCCCC1] | N-(2-(piperidin-1-yl)-4-(4- (trifluoromethyl)phenethyl) phenyl)octanamide |
| 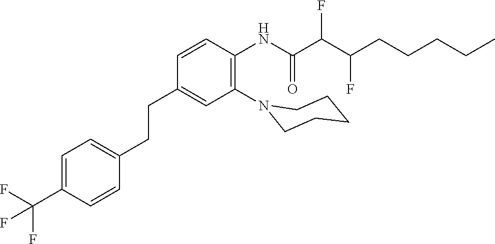 [SMILES: CCCCCC(F)C(F)C(=O)Nc1ccc(CCc2ccc(C(F)(F)F)cc2)cc1N1CCCCC1] | 2,3-difluoro-N-(2-(piperidin-1- yl)-4-(4- (trifluoromethyl)phenethyl) phenyl)octanamide (2R,3S)-2,3-difluoro-N-(2- (piperidin-1-yl)-4-(4- (trifluoromethyl)phenethyl) phenyl)octanamide (2S,3R)-2,3-difluoro-N-(2- (piperidin-1-yl)-4-(4- (trifluoromethyl)phenethyl) phenyl)octanamide (2R,3R)-2,3-difluoro-N-(2- (piperidin-1-yl)-4-(4- (trifluoromethyl)phenethyl) |
| | phenyl)octanamide |
| | (2S,3S)-2,3-difluoro-N-(2- |
| | (piperidin-1-yl)-4-(4- |
| | (trifluoromethyl)phenethyl) |
| | phenyl)octanamide |
| 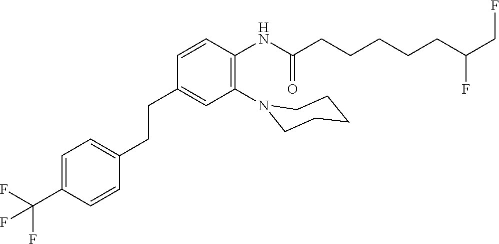 [SMILES: O=C(CCCCCC(F)CF)Nc1ccc(CCc2ccc(C(F)(F)F)cc2)cc1N1CCCCC1] | 7,8-difluoro-N-(2-(piperidin-1- yl)-4-(4- (trifluoromethyl)phenethyl) phenyl)octanamide (7R)-7,8-difluoro-N-(2- (piperidin-1-yl)-4-(4- (trifluoromethyl)phenethyl) phenyl)octanamide (7S)-7,8-difluoro-N-(2- (piperidin-1-yl)-4-(4- (trifluoromethyl)phenethyl) phenyl)octanamide |
| 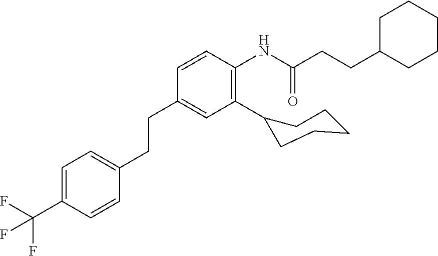 [SMILES: O=C(CCC1CCCCC1)Nc1ccc(CCc2ccc(C(F)(F)F)cc2)cc1C1CCCCC1] | 3-cyclohexyl-N-(2-cyclohexyl- 4-(4- (trifluoromethyl)phenethyl) phenyl)propanamide |
| 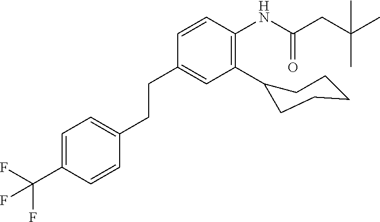 [SMILES: CC(C)(C)CC(=O)Nc1ccc(CCc2ccc(C(F)(F)F)cc2)cc1C1CCCCC1] | N-(2-cyclohexyl-4-(4- (trifluoromethyl)phenethyl) phenyl)-3,3-dimethylbutanamide |
| 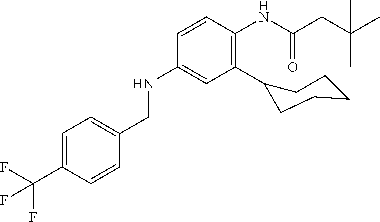 [SMILES: CC(C)(C)CC(=O)Nc1ccc(NCc2ccc(C(F)(F)F)cc2)cc1C1CCCCC1] | N-(2-cyclohexyl-4-((4- (trifluoromethyl)benzyl)amino) phenyl)-3,3-dimethylbutanamide |
| 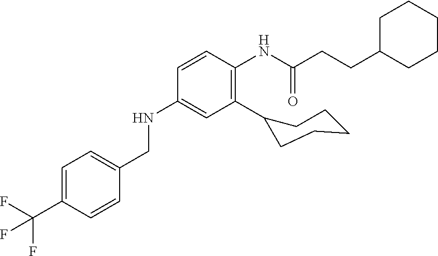 [SMILES: O=C(CCC1CCCCC1)Nc1ccc(NCc2ccc(C(F)(F)F)cc2)cc1C1CCCCC1] | 3-cyclohexyl-N-(2-cyclohexyl- 4-((4- (trifluoromethyl)benzyl)amino) phenyl)propanamide |
| 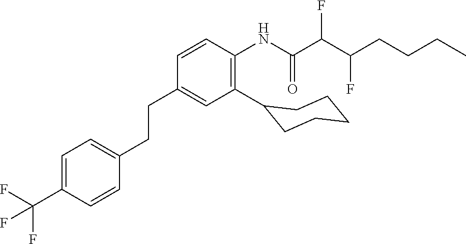 [SMILES: CCCCC(F)C(F)C(=O)Nc1ccc(CCc2ccc(C(F)(F)F)cc2)cc1C1CCCCC1] | N-(2-cyclohexyl-4-(4- (trifluoromethyl)phenethyl) phenyl)-2,3-difluoroheptanamide (2R,3S)-N-(2-cyclohexyl-4-(4- (trifluoromethyl)phenethyl) phenyl)-2,3-difluoroheptanamide (2S,3R)-N-(2-cyclohexyl-4-(4- (trifluoromethyl)phenethyl) phenyl)-2,3-difluoroheptanamide (2R,3R)-N-(2-cyclohexyl-4-(4- (trifluoromethyl)phenethyl) phenyl)-2,3-difluoroheptanamide (2S,3S)-N-(2-cyclohexyl-4-(4- (trifluoromethyl)phenethyl) phenyl)-2,3-difluoroheptanamide |
| 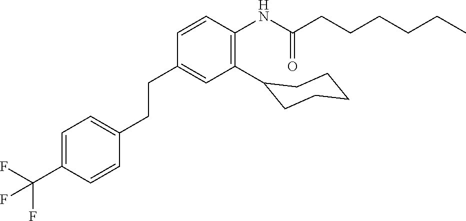 [SMILES: CCCCCCC(=O)Nc1ccc(CCc2ccc(C(F)(F)F)cc2)cc1C1CCCCC1] | N-(2-cyclohexyl-4-(4- (trifluoromethyl)phenethyl) phenyl)heptanamide |
| 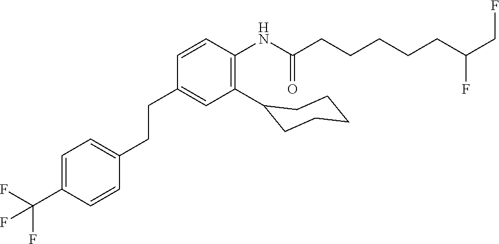 [SMILES: O=C(CCCCCC(F)CF)Nc1ccc(CCc2ccc(C(F)(F)F)cc2)cc1C1CCCCC1] | N-(2-cyclohexyl-4-(4- (trifluoromethyl)phenethyl) phenyl)-7,8-difluorooctanamide (7R)-N-(2-cyclohexyl-4-(4- (trifluoromethyl)phenethyl) phenyl)-7,8-difluorooctanamide (7S)-N-(2-cyclohexyl-4-(4- (trifluoromethyl)phenethyl) phenyl)-7,8-difluorooctanamide |
| 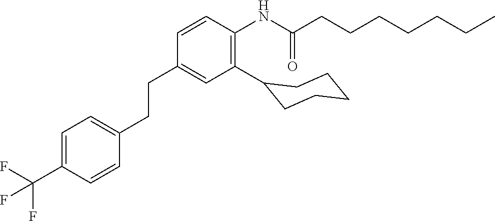 [SMILES: CCCCCCCC(=O)Nc1ccc(CCc2ccc(C(F)(F)F)cc2)cc1C1CCCCC1] | N-(2-cyclohexyl-4-(4- (trifluoromethyl)phenethyl) phenyl)octanamide |
| 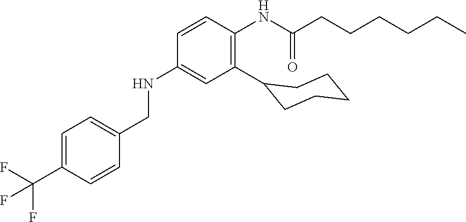 [SMILES: CCCCCCC(=O)Nc1ccc(NCc2ccc(C(F)(F)F)cc2)cc1C1CCCCC1] | N-(2-cyclohexyl-4-((4- (trifluoromethyl)benzyl)amino) phenyl)heptanamide |
| 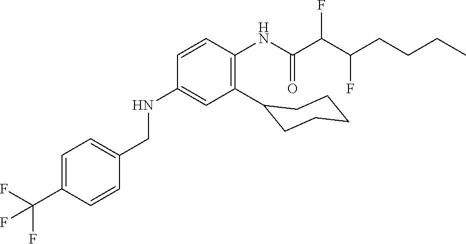 [SMILES: CCCCC(F)C(F)C(=O)Nc1ccc(NCc2ccc(C(F)(F)F)cc2)cc1C1CCCCC1] | N-(2-cyclohexyl-4-((4- (trifluoromethyl)benzyl)amino) phenyl)-2,3-difluoroheptanamide (2R,3S)-N-(2-cyclohexyl-4-((4- (trifluoromethyl)benzyl)amino) phenyl)-2,3-difluoroheptanamide (2S,3R)-N-(2-cyclohexyl-4-((4- (trifluoromethyl)benzyl)amino) phenyl)-2,3-difluoroheptanamide (2R,3R)-N-(2-cyclohexyl-4-((4- (trifluoromethyl)benzyl)amino) phenyl)-2,3-difluoroheptanamide (2S,3S)-N-(2-cyclohexyl-4-((4- (trifluoromethyl)benzyl)amino) phenyl)-2,3-difluoroheptanamide |
| 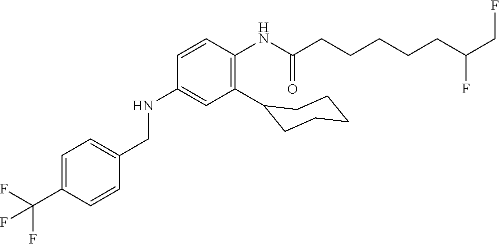 [SMILES: O=C(CCCCCC(F)CF)Nc1ccc(NCc2ccc(C(F)(F)F)cc2)cc1C1CCCCC1] | N-(2-cyclohexyl-4-((4- (trifluoromethyl)benzyl)amino) phenyl)-7,8-difluorooctanamide (7R)-N-(2-cyclohexyl-4-((4- (trifluoromethyl)benzyl)amino) phenyl)-7,8-difluorooctanamide (7S)-N-(2-cyclohexyl-4-((4- (trifluoromethyl)benzyl)amino) phenyl)-7,8-difluorooctanamide |
| 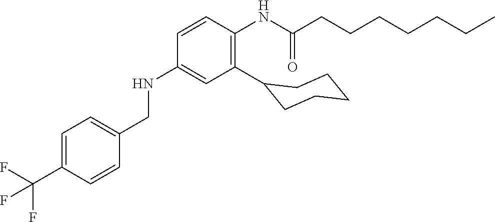 [SMILES: CCCCCCCC(=O)Nc1ccc(NCc2ccc(C(F)(F)F)cc2)cc1C1CCCCC1] | N-(2-cyclohexyl-4-((4- (trifluoromethyl)benzyl)amino) phenyl)octanamide |
| 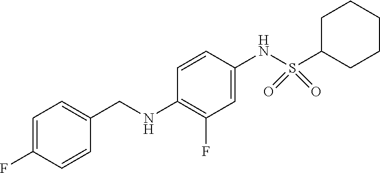 [SMILES: O=S(=O)(Nc1ccc(NCc2ccc(F)cc2)c(F)c1)C1CCCCC1] | N-(3-fluoro-4-((4- fluorobenzyl)amino)phenyl) cyclohexanesulfonamide |
| 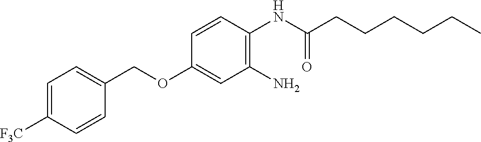 [SMILES: CCCCCCC(=O)Nc1ccc(OCc2ccc(C(F)(F)F)cc2)cc1N] | N-(2-amino-4-((4- (trifluoromethyl)benzyl)oxy) phenyl)heptanamide |
| 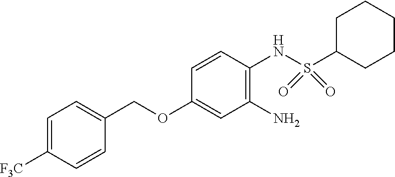 [SMILES: Nc1cc(OCc2ccc(C(F)(F)F)cc2)ccc1NS(=O)(=O)C1CCCCC1] | N-(2-amino-4-((4- (trifluoromethyl)benzyl)oxy) phenyl)cyclohexanesulfonamide |
| 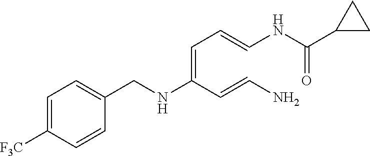 [SMILES: N/C=C/C(=C\C=C\NC(=O)C1CC1)NCc1ccc(C(F)(F)F)cc1] | N-(2-amino-4-((4- (trifluoromethyl)benzyl)amino) phenyl)cyclopropanecarboxamide |
| 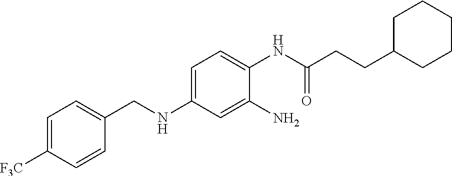 [SMILES: Nc1cc(NCc2ccc(C(F)(F)F)cc2)ccc1NC(=O)CCC1CCCCC1] | N-(2-amino-4-((4- (trifluoromethyl)benzyl)amino) phenyl)-3- cyclohexylpropanamide |
| 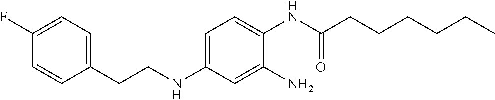 [SMILES: CCCCCCC(=O)Nc1ccc(NCCc2ccc(F)cc2)cc1N] | N-(2-amino-4-((4- fluorophenethyl)amino)phenyl) heptanamide |
| 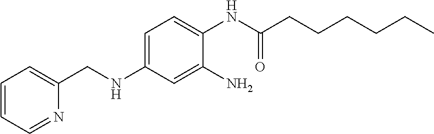 [SMILES: CCCCCCC(=O)Nc1ccc(NCc2ccccn2)cc1N] | N-(2-amino-4-((pyridin-2- ylmethyl)amino)phenyl) heptanamide |
| 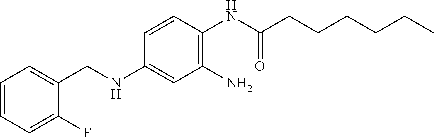 [SMILES: CCCCCCC(=O)Nc1ccc(NCc2ccccc2F)cc1N] | N-(2-amino-4-((2- fluorobenzyl)amino)phenyl) heptanamide |
| 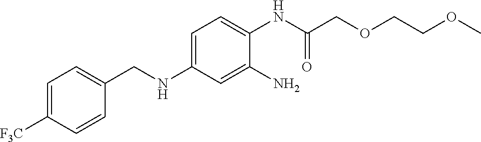 [SMILES: COCCOCC(=O)Nc1ccc(NCc2ccc(C(F)(F)F)cc2)cc1N] | N-(2-amino-4-((4- (trifluoromethyl)benzyl)amino) phenyl)-2-(2- methoxyethoxy)acetamide |
| 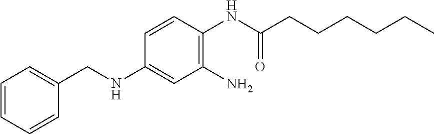 [SMILES: CCCCCCC(=O)Nc1ccc(NCc2ccccc2)cc1N] | N-(2-amino-4- (benzylamino)phenyl) heptanamide |
| 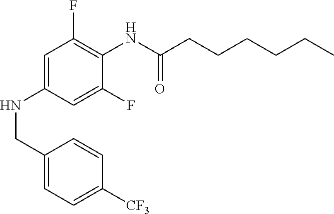 [SMILES: CCCCCCC(=O)Nc1c(F)cc(NCc2ccc(C(F)(F)F)cc2)cc1F] | N-(2,6-difluoro-4-((4- (trifluoromethyl)benzyl)amino) phenyl)heptanamide |
| 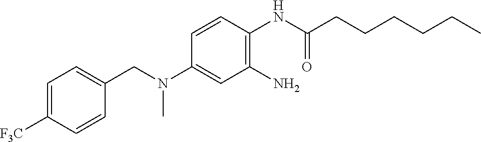 [SMILES: CCCCCCC(=O)Nc1ccc(N(C)Cc2ccc(C(F)(F)F)cc2)cc1N] | N-(2-amino-4-(methyl(4- (trifluoromethyl)benzyl)amino) phenyl)heptanamide |
| 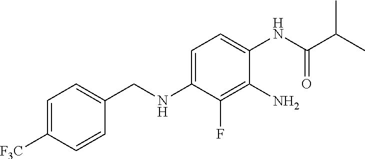 [SMILES: CC(C)C(=O)Nc1ccc(NCc2ccc(C(F)(F)F)cc2)c(F)c1N] | N-(2-amino-3-fluoro-4-((4- (trifluoromethyl)benzyl)amino) phenyl)isobutyramide |
| 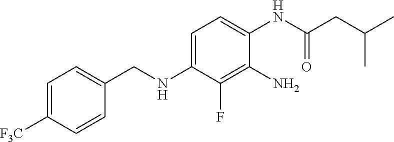 [SMILES: CC(C)CC(=O)Nc1ccc(NCc2ccc(C(F)(F)F)cc2)c(F)c1N] | N-(2-amino-3-fluoro-4-((4- (trifluoromethyl)benzyl)amino) phenyl)-3-methylbutanamide |
| 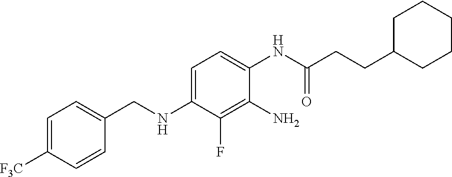 [SMILES: Nc1c(NC(=O)CCC2CCCCC2)ccc(NCc2ccc(C(F)(F)F)cc2)c1F] | N-(2-amino-3-fluoro-4-((4- (trifluoromethyl)benzyl)amino) phenyl)-3- cyclohexylpropanamide |
| 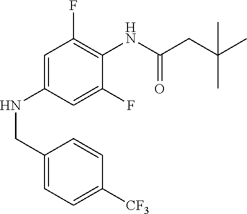 [SMILES: CC(C)(C)CC(=O)Nc1c(F)cc(NCc2ccc(C(F)(F)F)cc2)cc1F] | N-(2,6-difluoro-4-((4- (trifluoromethyl)benzyl)amino) phenyl)-3,3-dimethylbutanamide |
| 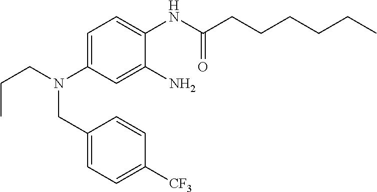 [SMILES: CCCCCCC(=O)Nc1ccc(N(CCC)Cc2ccc(C(F)(F)F)cc2)cc1N] | N-(2-amino-4-(propyl(4- (trifluoromethyl)benzyl)amino) phenyl)heptanamide |
| 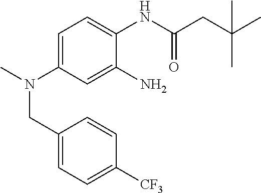 [SMILES: CN(Cc1ccc(C(F)(F)F)cc1)c1ccc(NC(=O)CC(C)(C)C)c(N)c1] | N-(2-amino-4-(methyl(4- (trifluoromethyl)benzyl)amino) phenyl)-3,3-dimethylbutanamide |
| 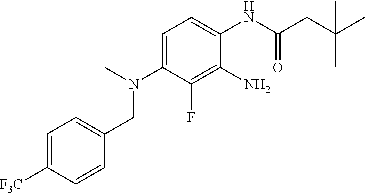 [SMILES: CN(Cc1ccc(C(F)(F)F)cc1)c1ccc(NC(=O)CC(C)(C)C)c(N)c1F] | N-(2-amino-3-fluoro-4- (methyl(4- (trifluoromethyl)benzyl)amino) phenyl)-3,3-dimethylbutanamide |
| 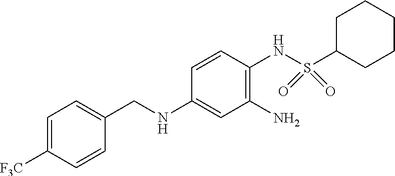 [SMILES: Nc1cc(NCc2ccc(C(F)(F)F)cc2)ccc1NS(=O)(=O)C1CCCCC1] | N-(2-amino-4-((4- (trifluoromethyl)benzyl)amino) phenyl)cyclohexanesulfonamide |
| 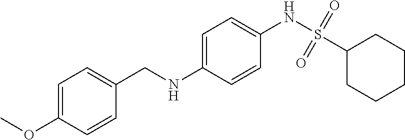 [SMILES: COc1ccc(CNc2ccc(NS(=O)(=O)C3CCCCC3)cc2)cc1] | N-(4-((4- methoxybenzyl)amino)phenyl) cyclohexanesulfonamide |
| 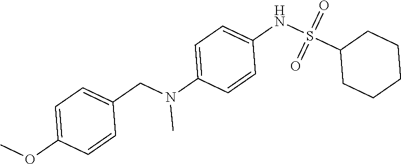 [SMILES: COc1ccc(CN(C)c2ccc(NS(=O)(=O)C3CCCCC3)cc2)cc1] | N-(4-((4- methoxybenzyl)(methyl)amino) pphenyl) cyclohexanesulfonamide |
| 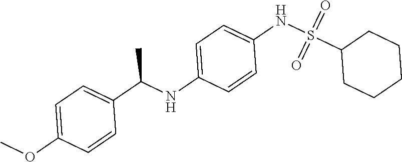 [SMILES: COc1ccc([C@@H](C)Nc2ccc(NS(=O)(=O)C3CCCCC3)cc2)cc1] | (R)-N-(4-((1-(4- methoxyphenyl)ethyl)amino) phenyl) cyclohexanesulfonamide |
| 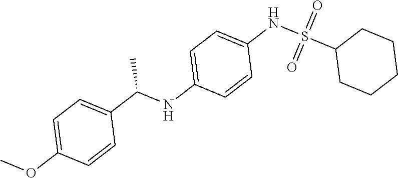 [SMILES: COc1ccc([C@H](C)Nc2ccc(NS(=O)(=O)C3CCCCC3)cc2)cc1] | (S)-N-(4-((1-(4- methoxyphenyl)ethyl)amino) phenyl) cyclohexanesulfonamide |
| 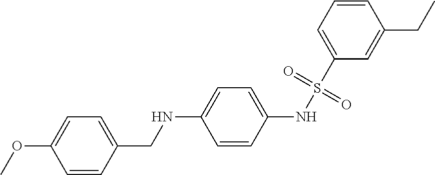 [SMILES: CCc1cccc(S(=O)(=O)Nc2ccc(NCc3ccc(OC)cc3)cc2)c1] | 3-ethyl-N-(4-((4- methoxybenzyl)amino)phenyl) benzenesulfonamide |

### Formula 56

In an embodiment, the Kv7 channel activator may be selected from one of the following compounds according to Formula 56. Such compounds are described in International Publication No. WO2020016297A1, published Jan. 23, 2020, and corresponding to International Application No. PCT/EP2019/069240 filed Jul. 17, 2019; Patent Cooperation Treaty application No. PCT/EP2019/069240 published Jan. 23, 2020, which is incorporated by reference in its entirety herein. In the case of any conflict of terminology in the context of Formula 56, this reference incorporated by reference herein controls.

In an embodiment, the Kv7 channel activator is a compound according to Formula 56:

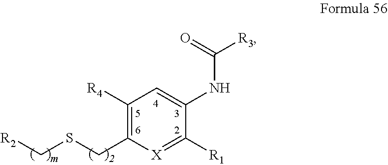

wherein, R₁ is selected from a branched or unbranched C1 to C5 alkyl group and —NR₁ₐR_{1b} group, where R₁ₐ and R_{1b} are selected independently of one another from hydrogen atom and branched or unbranched C1 to C5 alkyl group or with one another and with the nitrogen atom the NR₁ₐR_{1b} group form a C3 to C9 heterocycloalkyl group, the C3 to C9 heterocycloalkyl group each having at least one substituent selected from the hydrogen atom and C1 to C3 alkyl group and optionally in addition to the nitrogen atom of the NR₁ₐR_{1b}—Group has at least one further heteroatom in the cycle; R_{2 is} selected from the group consisting of branched or unbranched C1 to C10 alkyl group, C3 to C10 cycloalkyl group, C3 to C10 heterocycloalkyl group, at least one hetero atom being selected from nitrogen and oxygen atom, C6 to C12 aryl group; and C5 to C1 1 heteroaryl group, the C1 to C10 alkyl group, C3 to C10 cycloalkyl group, C3 to C10 heterocycloalkyl group, C6 to C12 aryl group, or C5 to C11 heteroaryl group in each case at least one further Substituents R₂ₐ, wherein R_{2a is} selected from the group consisting of hydrogen atom, halogen atom, branched or unbranched C1 to C3 alkyl group, branched or unbranched (per) halogenated C1 to C3 alkyl group and branched or unbranched C1 to C3 Alkoxy group, and wherein the C3 to C10 cycloalkyl group, C3 to C10 heterocycloalkyl group, C6 to C12 aryl group or C5 to C1 1 heteroaryl group comprises one or more ring systems which are condensed or separated; R_{3 is} selected from the group consisting of branched or unbranched C1 to C10 alkoxy group; branched or unbranched C1-C10 alkyl group and —(CH₂)_{P}-C6 to C12 aryl group, which has at least one substituent selected from hydrogen atom, halogen atom and C1 to C3 alkoxy group and wherein the C6 to C12 aryl group has one or comprises several ring systems which are condensed or separated; R₄ is selected from hydrogen, halogen atom and branched or unbranched C1-C5 alkyl group; X is a nitrogen atom or a —CH group; n is zero or an integer ranging from 1 to 3; m is zero or an integer ranging from 1 to 5; and p is zero or an integer ranging from 1 to 5.

In further embodiments, n is zero and/or m is 1 and/or X is a nitrogen atom.

In further embodiments, R¹ is selected from the group consisting of branched or unbranched C1- to C3-alkyl group and —NR₁ₐR_{1b} group, wherein R₁ₐ and R_{1b} are independently selected from hydrogen atom and branched or unbranched C1- to C3-alkyl group or with one another and form a C4 to C6 heterocycloalkyl group with the nitrogen atom of the NR₁ₐR_{1b} group, the C4 to C6 heterocycloalkyl group each having at least one substituent selected from the hydrogen atom and C1 to C3 alkyl group and optionally in addition to the nitrogen atom of the NR₁ₐR_{1b} group has at least one further oxygen atom in the cycle; and or R_{2 is} selected from the group consisting of mono- or difluorophenyl group; CF₃ phenyl group; F₅ S-phenyl group; C4 to C8 cycloalkyl group; branched or unbranched C1 to C5 alkyl group; Biphenyl group; Pyridine group; Piperidine group; Tetrahydropyran group; Dioxolane group and phenyl group, the phenyl group having at least one substituent R₂ₐ selected from hydrogen atom and methoxy group; and or R_{3 is} selected from the group consisting of ethoxy group, propyl group, tert-butyl group, —(CH₂)_{P}-phenyl group, which has at least one substituent selected from hydrogen atom, halogen atom and methoxy group and where p is zero or is 1 or 2; and or R_{4 is} a hydrogen atom, a bromine atom or a methyl group.

In further embodiments, the Kv7 channel activator is selected from the group consisting of:

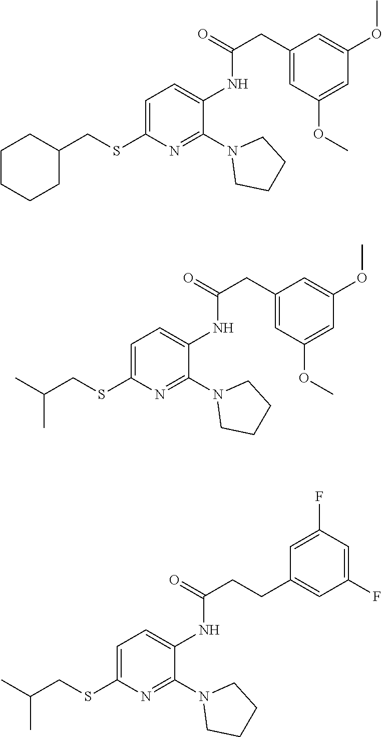

### Formulas 57-139

In another embodiment, the Kv7 channel activator may be selected from one of the following compounds of formulas 57-139. Such compounds are described in International Publication No. WO2018204765A1, published Nov. 8, 2018, and corresponding to International Application No. PCT/US2018/03 1057 filed May 4, 2018; which is incorporated by reference in its entirety herein. In the case of any conflict of terminology in the context of any of Formulas 57-139, this reference incorporated by reference herein controls.

In an embodiment, the Kv7 channel activator is a compound according to any one of formulas 57-139:

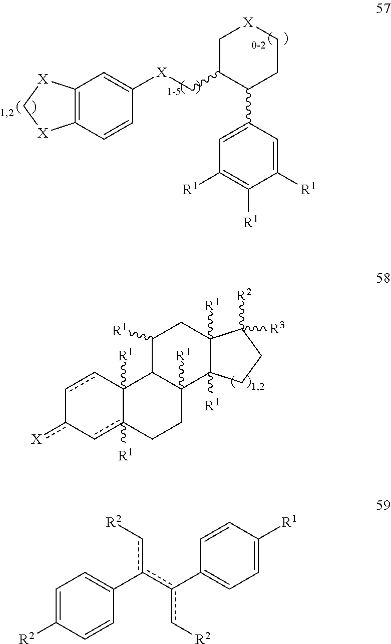

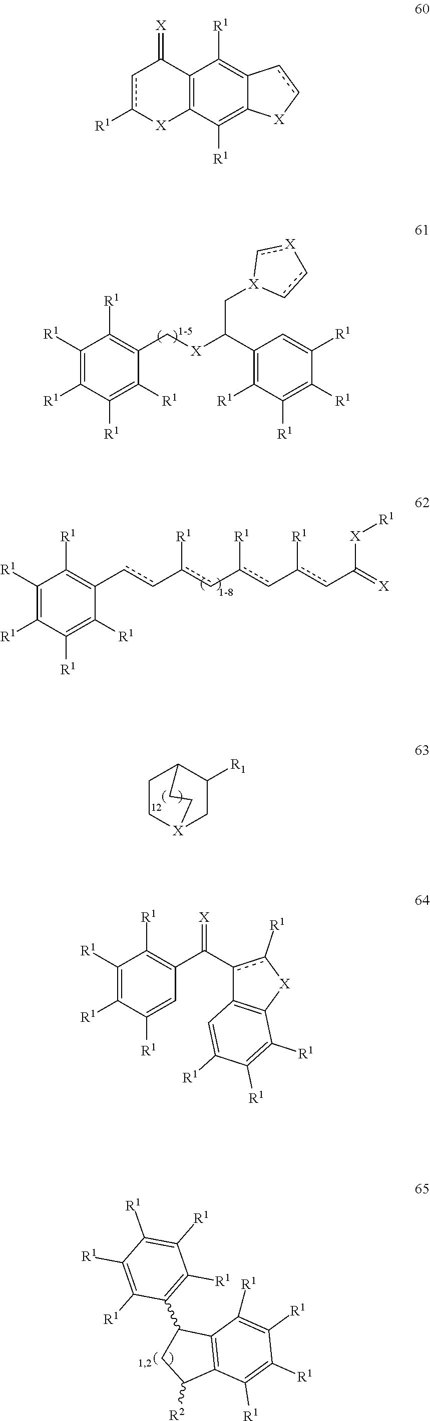

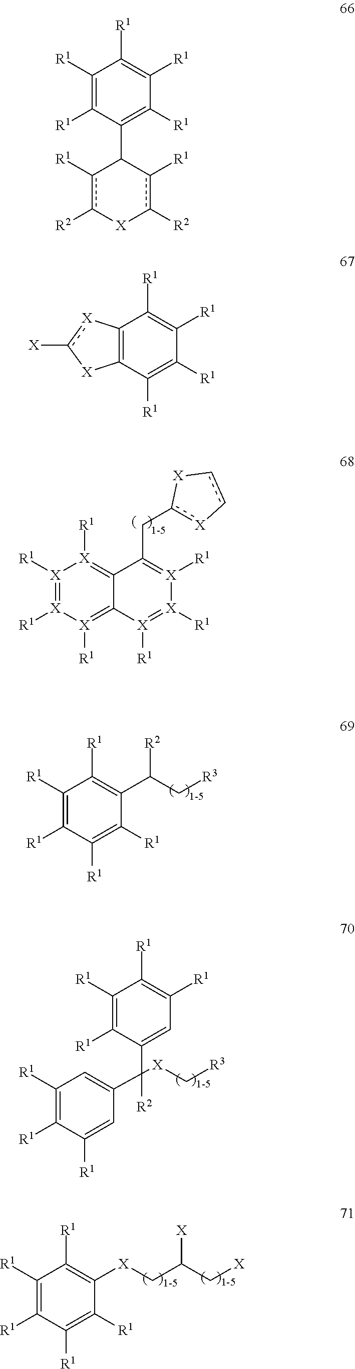

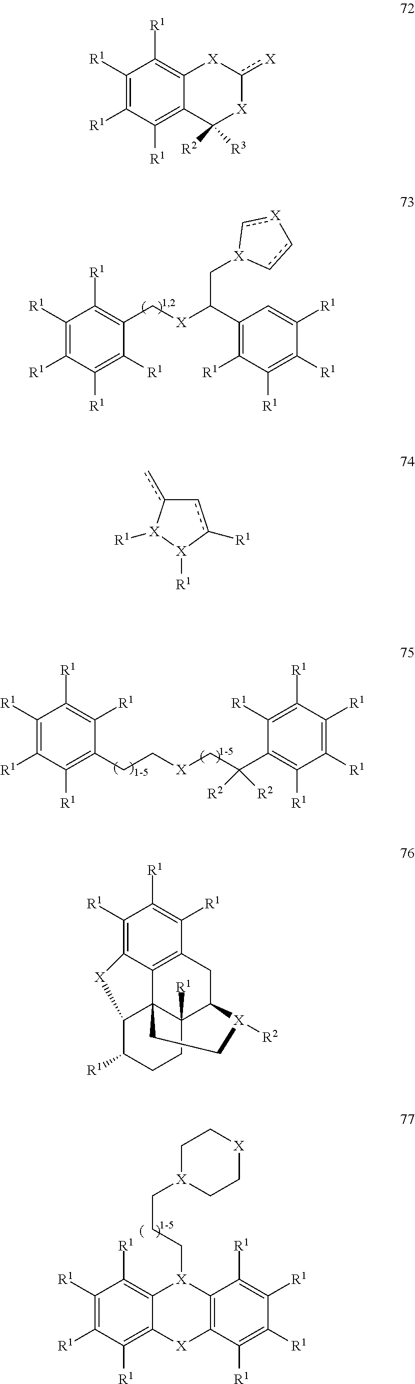

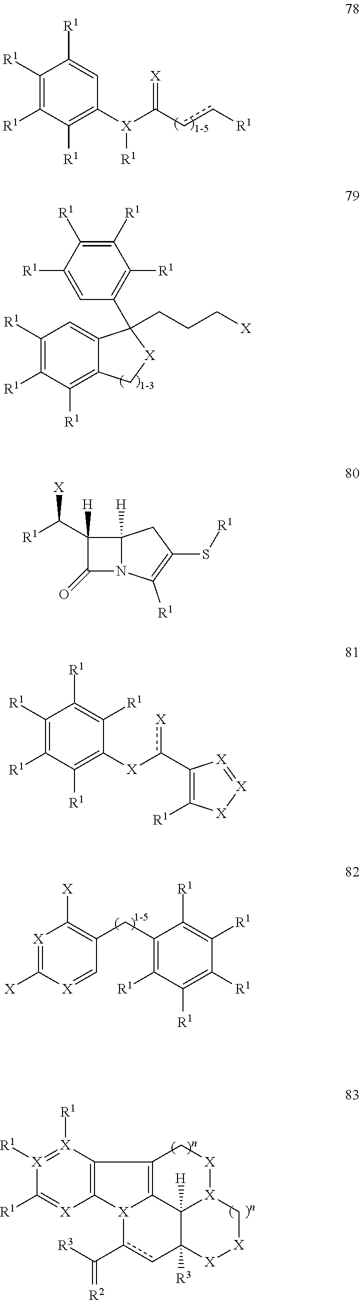

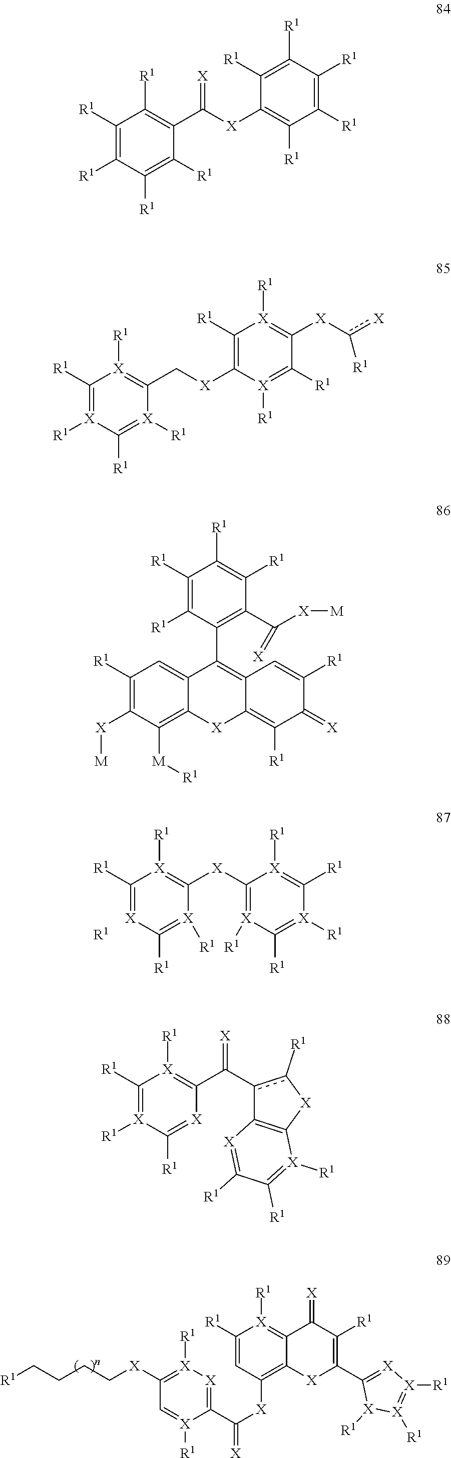

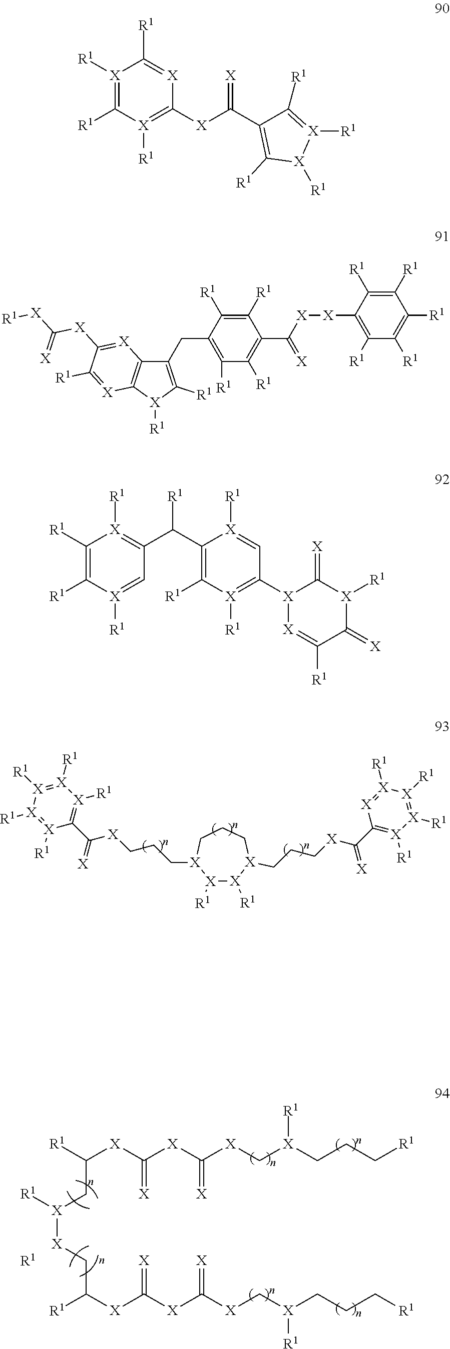

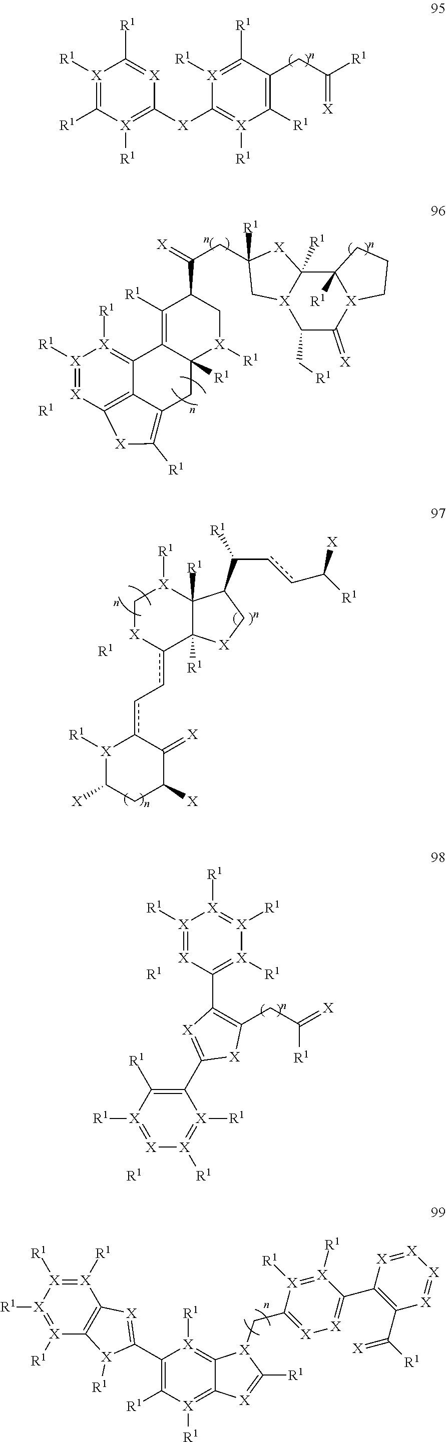

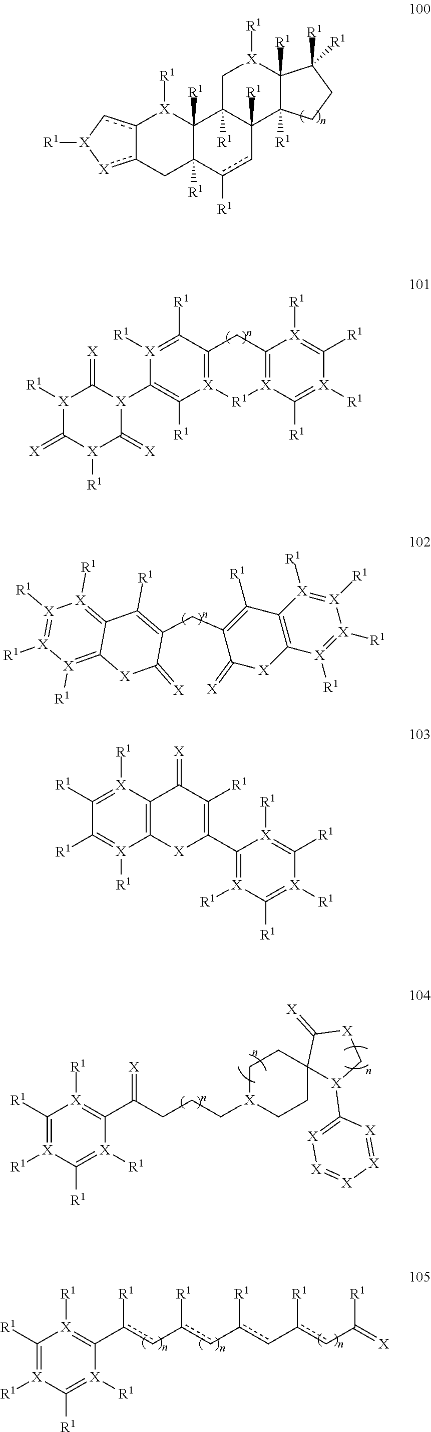

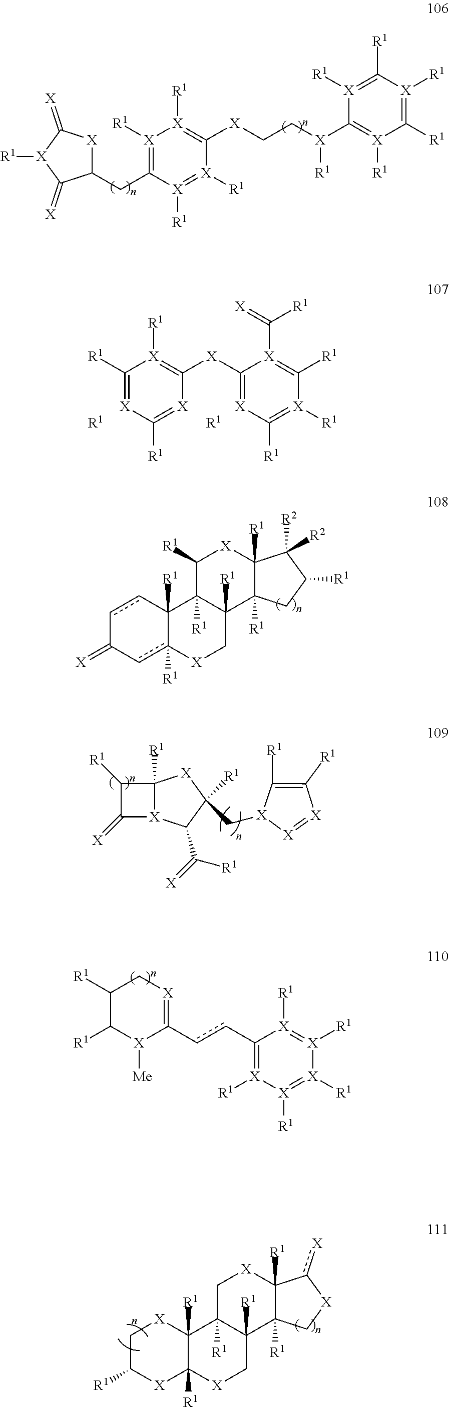

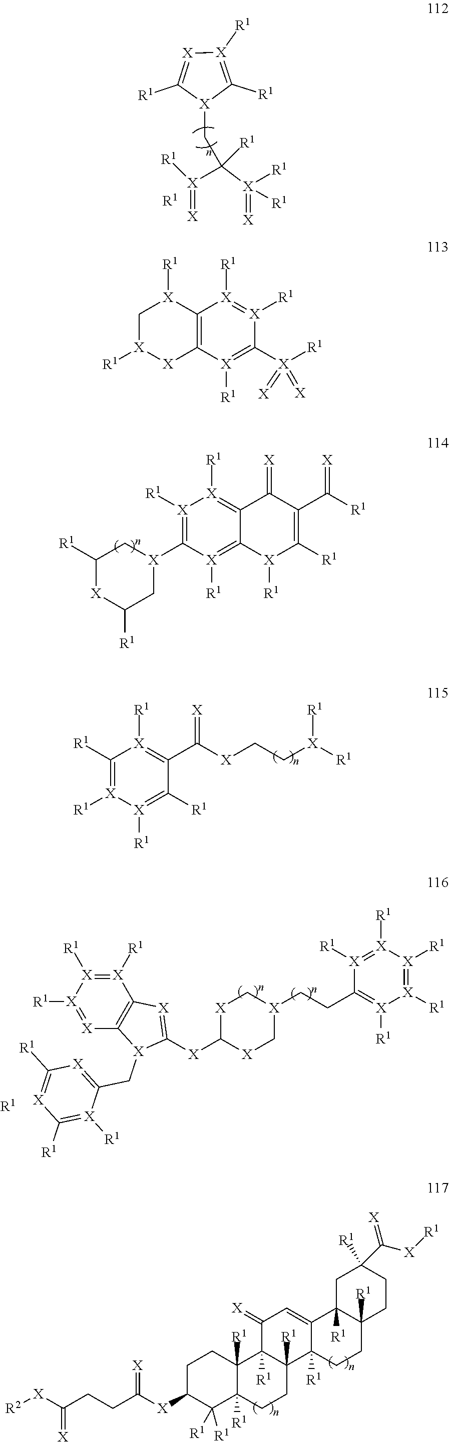

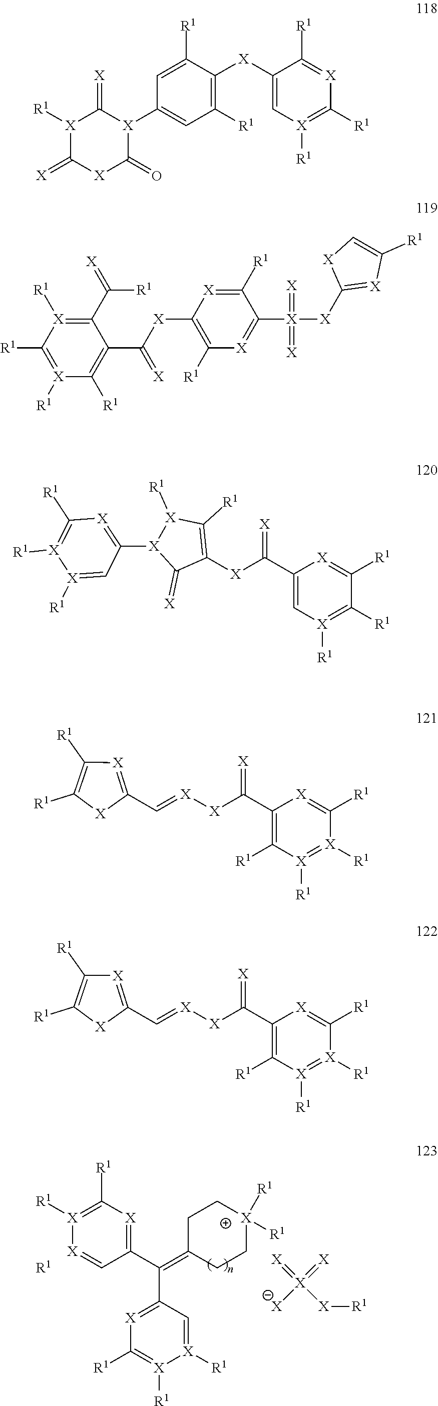

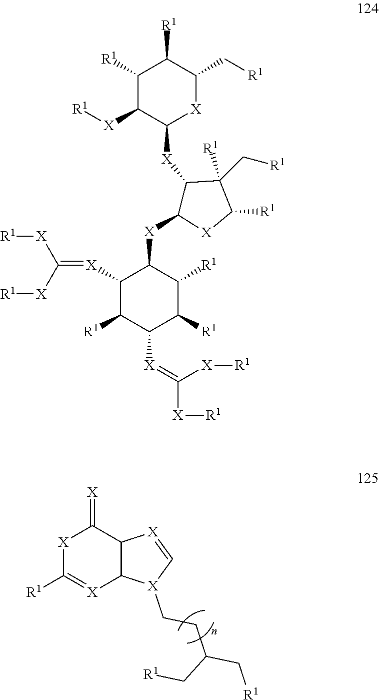

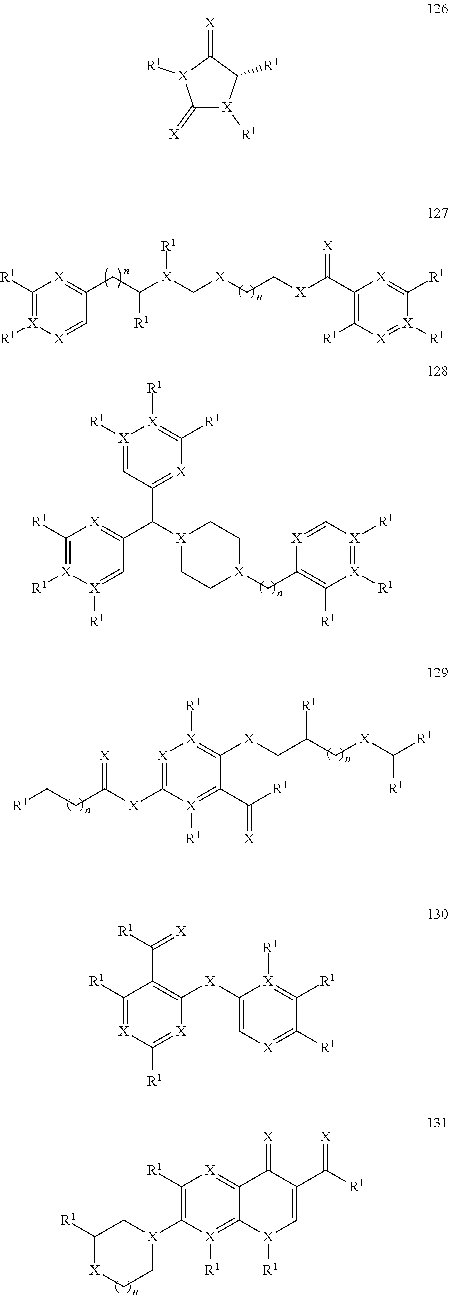

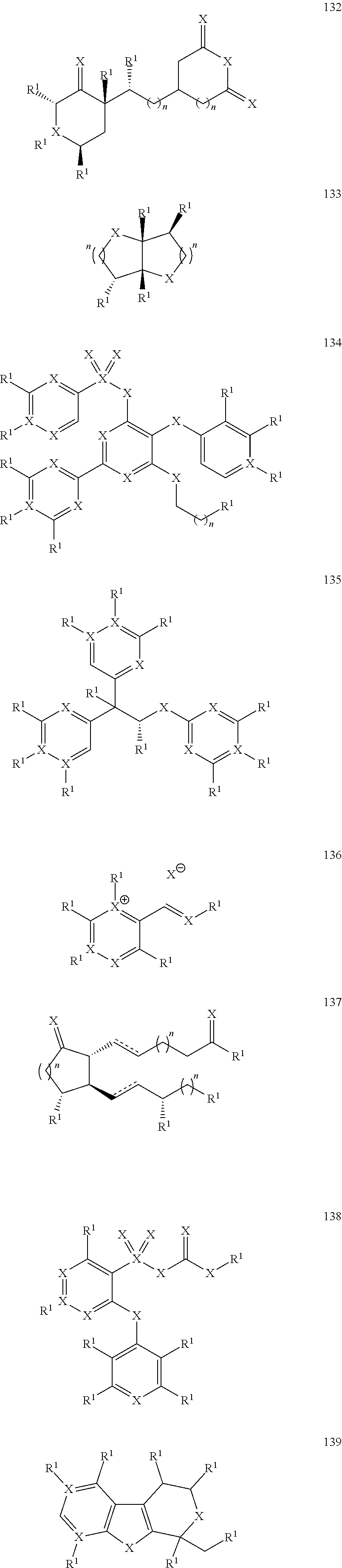

wherein (for any of compounds 57-139), each and any of R¹, R², and R³ independently ═H, CI, Br, F, I, OH, OAc, CF3, NH2, CN, OC₁₋₆ saturated, unsaturated alkyl, cycloalkyl, cycloheteroalkyl, or aryl, SC₁₋₆ saturated, unsaturated alkyl, cycloalkyl, cycloheteroalkyl, or aryl, C₁₋₆ saturated alkyl, unsaturated alkyl, cycloalkyl, cycloheteroalkyl, CO2H, COC1-8 alkyl unsaturated alkyl, or aryl, C02C₁₋₆ saturated, unsaturated alkyl, or aryl, CONH2, C02NHC₁₋₆ saturated, unsaturated alkyl or aryl, C02N(C₁₋₆ saturated, unsaturated alkyl or aryl)2, NHC₁₋₆ saturated, unsaturated alkyl, or cycloalkyl, N(C₁₋₆ saturated, unsaturated alkyl, or cycloalkyl)2, or C₅₋₇aryl or heteroaryl; and wherein each X=independently C, CH, CH2, S, SO, SO₂, NH, NC₁₋₆ saturated, unsaturated alkyl, or cycloalkyl, or O, and wherein each n, where present, is independently=0-6.

### Formula 140

In an embodiment, the Kv7 channel activator may be selected from one of the following compounds according to Formula 140. Such compounds are described in International Publication No. WO2016077724A1, published May 19, 2016, and corresponding to International Application No. PCT/US2015/060627 filed Nov. 13, 2015; U.S. Pat. No. 10,526,280, issued Jan. 7, 2020 and corresponding to US Application No. U.S. Ser. No. 10/526,280B2 filed Nov. 11, 2015; which are incorporated by reference in their entirety herein. In the case of any conflict of terminology in the context of Formula 140, this reference incorporated by reference herein controls.

In an embodiment, the Kv7 channel activator is a compound according to Formula 140:

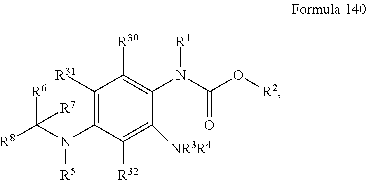

wherein, R¹ is H or optionally-substituted alkyl; R² is optionally-substituted alkyl; R³ and R⁴ are each independently H or optionally-substituted alkyl; R⁵ is H, optionally-substituted alkyl, acyl, or alkoxycarbonyl; R⁶ and R⁷ are each independently H, deuterium, optionally-substituted alkyl, or R⁶ and R⁷ together form a carbocycle; R⁸ is optionally-substituted thiazolyl, optionally-substituted thiophenyl, or substituted phenyl, provided that if R⁸ is 4-halophenyl, then R² is substituted alkyl or branched alkyl or at least one of R⁶ or R⁷ is not H; and R³⁰, R³¹ and R³² are each independently H, deuterium, halogen, substituted sulfanyl, or optionally-substituted alkoxy.

In further embodiments, R¹ is H.

In further embodiments, R² is C₁-C₆ alkyl.

In further embodiments, R² is carbocyclic-substituted alkyl or heterocyclic-substituted alkyl.

In further embodiments, R³ and R⁴ are each H.

In further embodiments, R⁵ is H.

In further embodiments, R⁶ and R⁷ are both H; R⁶ and R⁷ are both deuterium; R⁶ and R⁷ together form a cycloalkyl; or at least one of R⁶ or R⁷ is a substituted alkyl.

In further embodiments, R⁸ is selected from:

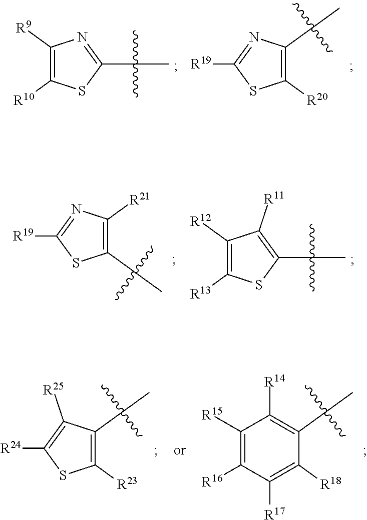

wherein R⁹-R²⁵ are each independently H, halogen, optionally-substituted sulfanyl, or optionally-substituted alkyl.

In further embodiments, R⁸ is

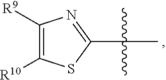

wherein at least one of R⁹ or R¹⁰ is halogen.

In further embodiments, R⁸ is

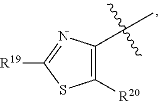

wherein at least one of R¹⁹ or R²⁰ is halogen.

In further embodiments, R⁸ is

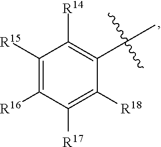

wherein at least one of R¹⁴-R¹⁸ is substituted sulfanyl or haloalkyl; or wherein R¹⁶ is F.

In further embodiments, at least one of R¹⁴-R¹⁸ is —SF₅ or CF₃.

In further embodiments, R⁸ is

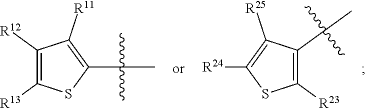

wherein at least one of R¹¹-R¹³ or R²³-R²⁵ is substituted sulfanyl.

In further embodiments, R⁸ is

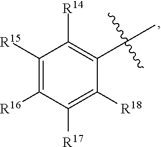

wherein at least one of R¹⁴-R¹⁸ is halo-substituted sulfanyl, haloalkyl or halogen.

In further embodiments, at least one of R¹⁴-R¹⁸ is —SF₅ or —CF₃.

In further embodiments, R¹⁵, R¹⁶, or R¹⁷ is —SF₅ or —CF₃.

In further embodiments, R³² is —F, and R³⁰ and R³¹ are each H.

In further embodiments, the Kv7 Channel activator is selected from the group consisting of:

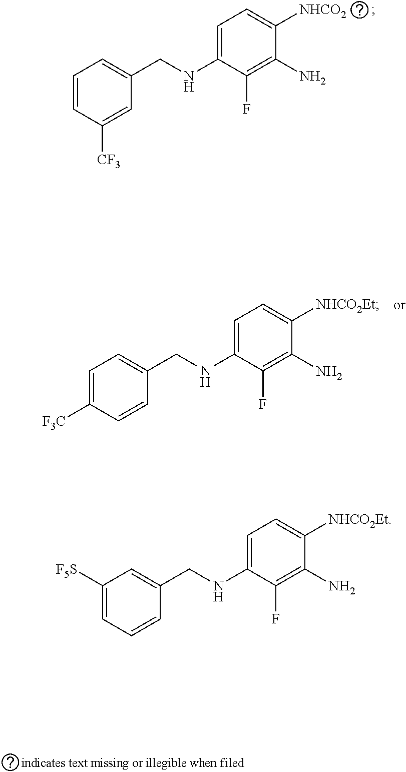

In further embodiments, the compound is according to formula 141 wherein: R¹ is H or optionally-substituted alkyl; R² is substituted alkyl having one or more hydrogen atoms substituted with a substituent selected from halogen, cycloalkyl, alkoxy, amino, hydroxyl, aryl, alkenyl, or carboxyl; R³ and R⁴ are each independently H or optionally-substituted alkyl; R⁵ is H, optionally-substituted alkyl, acyl, or alkoxycarbonyl; R⁶ and R⁷ are each independently H, deuterium, optionally-substituted alkyl, or R⁶ and R⁷ together form a carbocycle; R⁸ is optionally-substituted thiazolyl, optionally-substituted thiophenyl, or substituted phenyl; and R³⁰, R³¹ and R³² are each independently H, deuterium, halogen, substituted sulfanyl, or optionally-substituted alkoxy.

In further embodiments, the compound is according to formula 141 wherein: R¹ is H or optionally-substituted alkyl; R² is optionally-substituted alkyl; R³ and R⁴ are each independently H or optionally-substituted alkyl; R⁵ is H, optionally-substituted alkyl, acyl, or alkoxycarbonyl; R⁶ and R⁷ are each independently H, deuterium, optionally-substituted alkyl, or R⁶ and R⁷ together form a carbocycle, provided at least one R⁶ or R⁷ is not H; R⁸ is optionally-substituted thiazolyl, optionally-substituted thiophenyl, or substituted phenyl, provided that if R⁸ is 4-halophenyl, then R² is substituted alkyl or branched alkyl or at least one of R⁶ or R⁷ is not H; and R³⁰, R³¹ and R³² are each independently H, deuterium, halogen, substituted sulfanyl, or optionally-substituted alkoxy.

In further embodiments, the compound is according to formula 141 wherein: R¹ is H or optionally-substituted alkyl; R² is optionally-substituted alkyl; R³ and R⁴ are each independently H or optionally-substituted alkyl; R⁵ is H, optionally-substituted alkyl, acyl, or alkoxycarbonyl; R⁶ and R⁷ are each independently H, deuterium, optionally-substituted alkyl, or R⁶ and R⁷ together form a carbocycle; R⁸ is

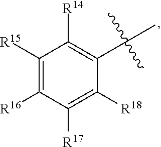

wherein at least one of R¹⁴-R¹⁸ is substituted sulfanyl or haloalkyl; and R³ and R³¹ are each independently H, deuterium, halogen, substituted sulfanyl, or optionally-substituted alkoxy; and R³² is deuterium, halogen, substituted sulfanyl, or optionally-substituted alkoxy.

In further embodiments, R³² is halogen.

In further embodiments, R³² is —F.

In further embodiments, at least one of R¹⁴-R¹⁸ is halo-substituted sulfanyl.

In further embodiments, at least one of R¹⁴-R¹⁸ is haloalkyl.

In further embodiments, at least one of R¹⁴-R¹⁸ is —SF₅.

In further embodiments, at least one of R¹⁴-R¹⁸ is —CF₃.

In further embodiments, R¹⁶ is —SF₅.

In further embodiments, R¹⁶ is —CF₃.

In further embodiments, R² is alkyl.

In further embodiments, R² is C₁-C₆ alkyl.

In further embodiments, R² is ethyl.

In further embodiments, R³, R⁴, R⁵, R⁶, and R⁷ are each H.

In further embodiments, R³² is —F and R¹⁶ is —CF₃.

In further embodiments, R² is alkyl.

In further embodiments, R³ and R³¹ are each H.

In further embodiments, R¹⁵, R¹⁶, or R¹⁷ is —SF₅ or —CF₃.

In further embodiments, R¹, R¹, R³, R⁴, R⁵, R⁶, and R⁷ are each H; R² is alkyl; and R³² is —F.

### Formula 141

In an embodiment, the Kv7 channel activator may be selected from one of the following compounds according to Formula 141. Such compounds are described in International Publication No. WO2016077724A1, published May 19, 2016, and corresponding to International Application No. PCT/US2015/060627 filed Nov. 13, 2015; U.S. Pat. No. 10,526,280, issued Jan. 7, 2020, and corresponding to US Application No. U.S. Ser. No. 10/526,280B2 filed Nov. 11, 2015; which are incorporated by reference in their entirety herein. In the case of any conflict of terminology in the context of Formula 141, these references incorporated by reference herein control.

In an embodiment, the Kv7 channel activator is a compound according to Formula 141:

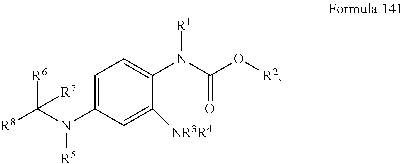

wherein, R¹ is H or optionally-substituted alkyl; R² is optionally-substituted alkyl; R³ and R⁴ are each independently H or optionally-substituted alkyl; R⁵ is H, optionally-substituted alkyl, acyl, or alkoxycarbonyl; R⁶ and R⁷ are each independently H, deuterium, optionally-substituted alkyl, or R⁶ and R⁷ together form a carbocycle; and R⁸ is optionally-substituted thiazolyl.

In further embodiments, the compound is according to formula 142 wherein: R¹ is H or optionally-substituted alkyl; R² is optionally-substituted alkyl; R³ and R⁴ are each independently H or optionally-substituted alkyl; R⁵ is H, optionally-substituted alkyl, acyl, or alkoxycarbonyl; R⁶ and R⁷ are each independently H, deuterium, optionally-substituted alkyl, or R⁶ and R⁷ together form a carbocycle; and R⁸ is substituted thiophenyl, wherein at least one substituent on the thiophenyl is halo-substituted sulfanyl.

### Formula 142

In an embodiment, the Kv7 channel activator may be selected from one of the following compounds according to Formula 142. Such compounds are described in U.S. Pat. No. 10,077,245, issued Sep. 18, 2018, and corresponding to U.S. application Ser. No. 15/306,971 filed Apr. 22, 2015; U.S. Pat. No. 10,316,008, issued Jun. 11, 2019 and corresponding to U.S. application Ser. No. 15/870,276 filed Jan. 12, 2018; international Publication No. WO2015165352A1, published Nov. 5, 2015 and corresponding to International Application No. PCT/CN2015/077216 filed Apr. 22, 2015; which are incorporated by reference in their entirety herein. In the case of any conflict of terminology in the context of Formula 142, these references incorporated by reference herein control.

In an embodiment, the Kv7 channel activator is a compound according to Formula 142:

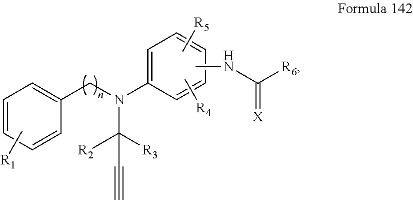

wherein, X is selected from a group consisting of oxygen and sulfur; n is 1, 2 or 3; R₁ is H or halogen; R₂ and R₃ are each independently selected from a group consisting of H, D and C₁-C₃ alkyl; or R₂ and R₃ together with the carbon atom to which they attached form C₃-C₆ saturated ring; R₄ and R₅ are each independently selected from a group consisting of H; halogen; C₁-C₆ alkyl; C₃-C₆ cycloalkyl; cyano; alkoxyl; C₁-C₆ alkyl substituted by hydroxy, amino, C₁-C₄ alkoxy, C₁-C₄ alkylcarbonyl, or halogen; C₁-C₄ alkoxy substituted by halogen; C₁-C₆ alkylcarbonyl; C₁-C₆ alkoxycarbonyl; C₁-C₆ alkylaminocarbonyl; C₂-C₆ alkenyl; and C₂-C₆ alkynyl; provided that R₄ and R₅ are not simultaneously hydrogen; R₆ is selected from a group consisting of C₁-C₆ alkoxy; C₁-C₆ alkylamino; C₁-C₆ alkyl; C₃-C₆ cycloalkyl; C₂-C₆ alkenyl; C₂-C₆ alkynyl; C₆-C₁₀ aryl; C₁-C₆ alkyl substituted by halogen, cyano, hydroxy, C₁-C₆ alkoxyl, di(C₁-C₄ alkyl)amino, C₁-C₆ alkylcarbonyl, C₁-C₆ alkylcarbonylamino, or C₁-C₆ alkoxycarbonyl; C₃-C₆ cycloalkyl substituted by halogen; C₂-C₆ alkenyl substituted by halogen; C₂-C₆ alkynyl substituted by halogen; tetrahydrofuranyl; and

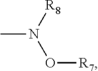

wherein, R₇ and R₈ are each independently selected from a group consisting of C₁-C₄ alkyl.

In further embodiments, R₁ is H or fluorine; R₂ and R₃ are each independently selected from a group consisting of H and D, or R₂ and R₃ together with the carbon atom to which they attached form cyclopropyl; one of R₄ and R₅ is C₁-C₄ alkyl, and the other is H or C₁-C₄ alkyl. In further embodiments, one of R₄ and R₅ is methyl, and the other is H or methyl.

### Formula 143

In an embodiment, the Kv7 channel activator may be selected from one of the following compounds according to Formula 143. Such compounds are described in U.S. Pat. No. 10,077,245, issued Sep. 18, 2018, and corresponding to U.S. application Ser. No. 15/306,971 filed Apr. 22, 2015; U.S. Pat. No. 10,316,008, issued Jun. 11, 2019 and corresponding to U.S. application Ser. No. 15/870,276 filed Jan. 12, 2018; international Publication No. WO2015165352A1, published Nov. 5, 2015, and corresponding to International Application No. PCT/CN2015/077216 filed Apr. 22, 2015; which are incorporated by reference in their entirety herein. In the case of any conflict of terminology in the context of Formula 143, these references incorporated by reference herein control.

In an embodiment, the Kv7 channel activator is a compound according to Formula 143:

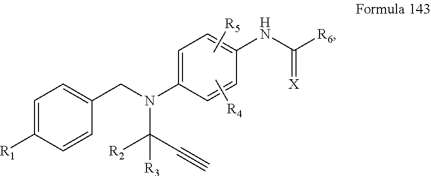

wherein, X: is selected from a group consisting of oxygen and sulfur; R₁ is H or
halogen; R₂ and R₃ are each independently selected from a group consisting of H, D and C₁-C₃ alkyl; or R₂ and R₃ together with the carbon atom to which they attached form C₃-C₆ saturated ring; R₄ and R₅ are each independently selected from a group consisting of H; halogen; C₁-C₆ alkyl; C₃-C₆ cycloalkyl; cyano; C₁-C₄ alkoxyl; C₁-C₆ alkyl substituted by hydroxy, amino, C₁-C₄ alkoxy, C₁-C₄ alkylcarbonyl, or halogen; C₁-C₄ alkoxy substituted by halogen; C₁-C₆ alkylcarbonyl; C₁-C₆ alkoxycarbonyl; C₁-C₆ alkylaminocarbonyl; C₂-C₆ alkenyl; and C₂-C₆ alkynyl; provided that R₄ and R₅ are not simultaneously hydrogen; R₆ is selected from a group consisting of C₁-C₆ alkoxy; C₁-C₆ alkylamino; C₁-C₆ alkyl; C₃-C₆ cycloalkyl; C₂-C₆ alkenyl; C₂-C₆ alkynyl; C₆-C₁₀ aryl; C₁-C₆ alkyl substituted by halogen, cyano, hydroxy, C₁-C₆ alkoxyl, di(C₁-C₄ alkyl)amino, C₁-C₆ alkylcarbonyl, C₁-C₆ alkylcarbonylamino, or C₁-C₆ alkoxycarbonyl; C₃-C₆ cycloalkyl substituted by halogen; C₂-C₆ alkenyl substituted by halogen; C₂-C₆ alkynyl substituted by halogen; tetrahydrofuranyl; and

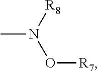

wherein, R₇ and R₈ are each independently selected from a group consisting of C₁-C₄ alkyl.

In further embodiments, R₁ is H or fluorine; R₂ and R₃ are each independently selected from a group consisting of H and D, or R₂ and R₃ together with the carbon atom to which they attached form cyclopropyl; one of R₄ and R₅ is C₁-C₄ alkyl, and the other is H or C₁-C₄ alkyl.

### Formula 144-146

In an embodiment, the Kv7 channel activator may be selected from one of the following compounds according to any one of Formulas 144-146. Such compounds are described in U.S. Pat. No. 10,077,245, issued Sep. 18, 2018, and corresponding to U.S. application Ser. No. 15/306,971 filed Apr. 22, 2015; U.S. Pat. No. 10,316,008, issued Jun. 11, 2019, and corresponding to U.S. application Ser. No. 15/870,276 filed Jan. 12, 2018; international Publication No. WO2015165352A1, published Nov. 5, 2015, and corresponding to International Application No. PCT/CN2015/077216 filed Apr. 22, 2015; which are incorporated by reference in their entirety herein. In the case of any conflict of terminology in the context of Formulas 144-146, these references incorporated by reference herein control.

In an embodiment, the Kv7 channel activator is a compound according to a formula from the group consisting of 144, 145, and 146:

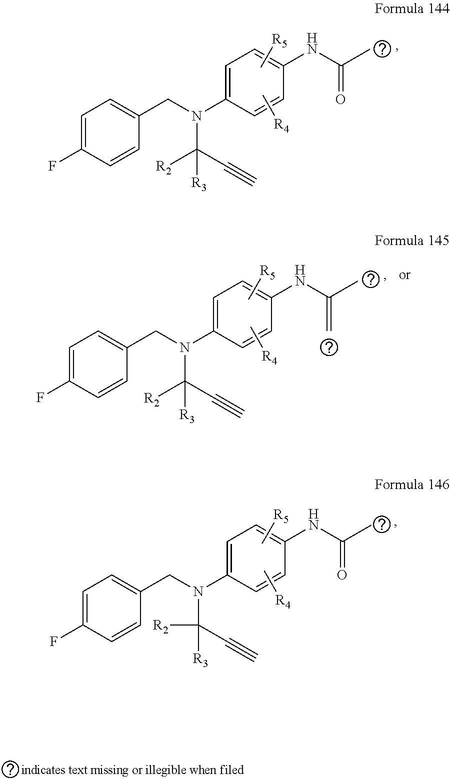

wherein, R₂ and R₃ are each independently selected from a group consisting of H, D and C₁-C₃ alkyl; or R₂ and R₃ together with the carbon atom to which they attached form C₃-C₆ saturated ring; R₄ and R₅ are each independently selected from a group consisting of H; C₁-C₆ alkyl; C₁-C₄ alkoxyl; C₁-C₆ alkyl substituted by halogen; C₁-C₄ alkoxy substituted by halogen; provided that R₄ and R₅ are not simultaneously hydrogen; R₉ is selected from a group consisting of C₁-C₆ alkyl and C₃-C₆ cycloalkyl; R₁₀ is selected from a group consisting of C₁-C₆ alkyl; C₁-C₆ alkyl substituted by halogen, cyano, hydroxy, C₁-C₆ alkoxyl, di(C₁-C₄ alkyl)amino, C₁-C₆ alkylcarbonyl, C₁-C₆ alkylamido, or C₁-C₆ alkoxycarbonyl; C₃-C₆ cycloalkyl; and C₃-C₆ cycloalkyl substituted by halogen; tetrahydrofuranyl; and

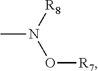

wherein, R₇ and R₈ are each independently selected from a group consisting of C₁-C₄ alkyl.

In further embodiments, R₂ and R₃ are each independently selected from a group consisting of H and D, or R₂ and R₃ together with the carbon atom to which they attached form cyclopropyl; one of R₄ and R₅ is C₁-C₁ alkyl, and the other is H or C₁-C₄ alkyl; R₉ is selected from a group consisting of C₁-C₆ alkyl and C₃-C₆ cycloalkyl; R₁₀ is selected from a group consisting of C₁-C₆ alkyl; C₁-C₆ alkyl substituted by halogen, cyano, hydroxy, C₁-C₆ alkoxyl, di(C₁-C₄ alkyl)amino, C₁-C₆ alkylcarbonyl, C₁-C₆ alkylamido, or C₁-C₆ alkoxycarbonyl; C₃-C₆ cycloalkyl; C₃-C₆ cycloalkyl substituted by halogen; tetrahydrofuranyl; and

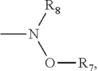

wherein, R₇ and R₈ are each independently selected from a group consisting of C₁-C₄ alkyl.

In further embodiments, R₉ is selected from a group consisting of methyl, ethyl and propyl; R₁₀ is selected from a group consisting of C₁-C₃ alkyl; C₁-C₃ alkyl substituted by halogen, cyano, hydroxy, C₁-C₃ alkoxyl, di(C₁-C₃ alkyl)amino, C₁-C₃ alkylcarbonyl, C₁-C₃ alkylamido, or C₁-C₃ alkoxycarbonyl; C₃-C₆ cycloakyl; C₃-C₆ cycloakyl substituted by halogen; tetrahydrofuranyl; and

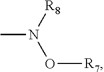

wherein, R₇ and R₈ are each independently selected from a group consisting of C₁-C₃ alkyl.

In further embodiments, the Kv7 Channel activator is selected from the group consisting of:

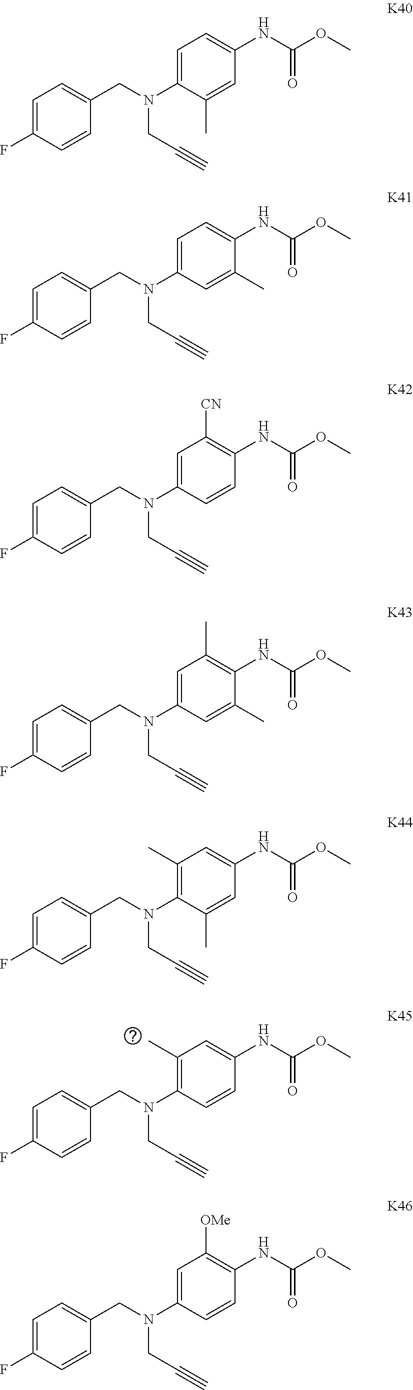

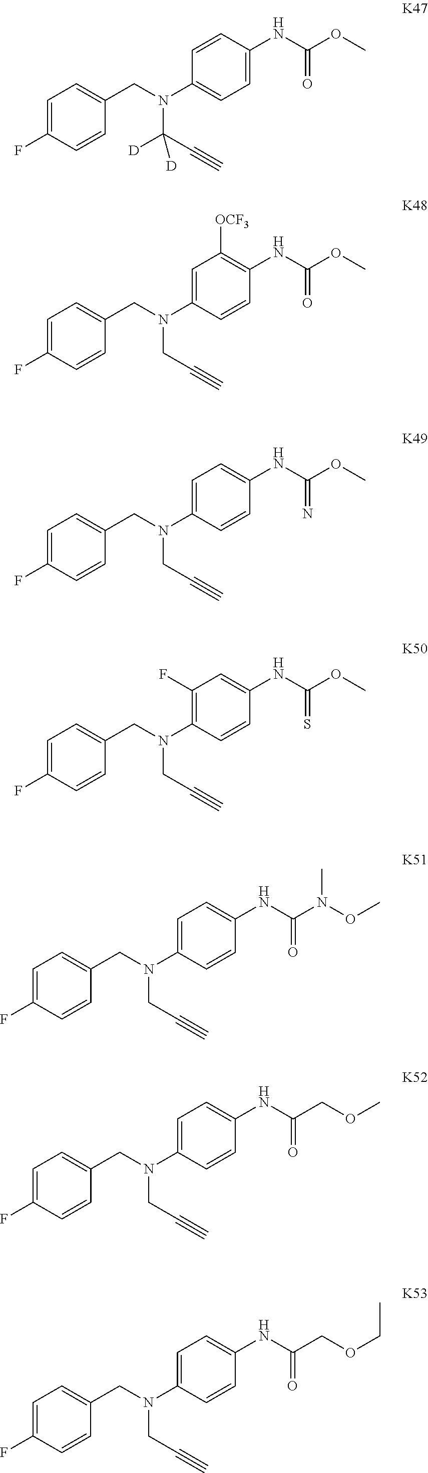

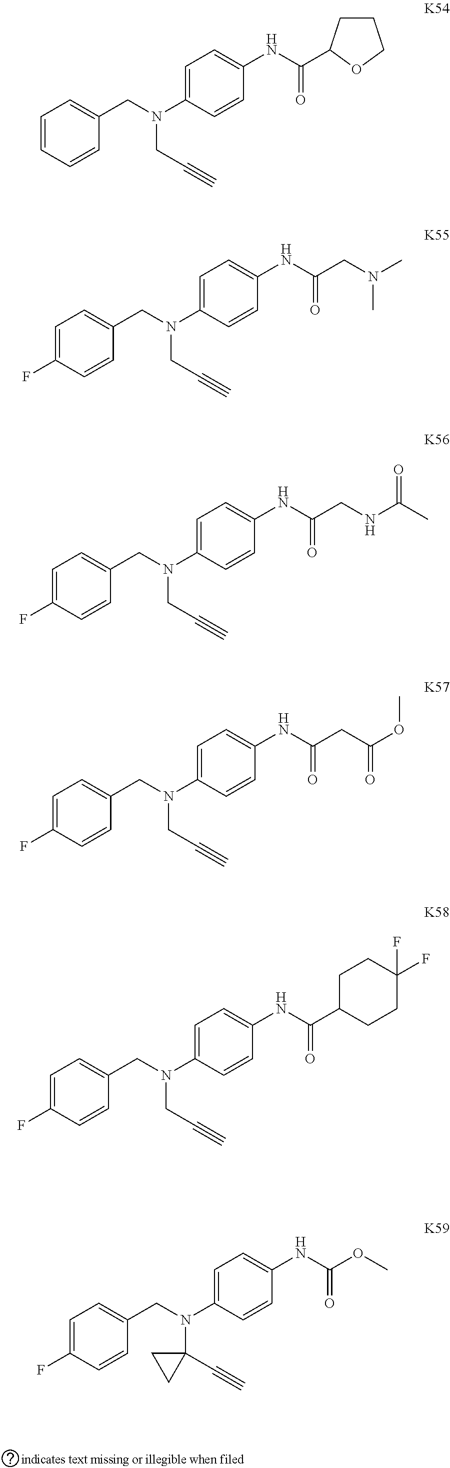 [SMILES: C#CCN(Cc1ccccc1)c1ccc(NC(=O)C2CCCO2)cc1 ; C#CCN(Cc1ccc(F)cc1)c1ccc(NC(=O)CN(C)C)cc1 ; C#CCN(Cc1ccc(F)cc1)c1ccc(NC(=O)CNC(C)=O)cc1 ; C#CCN(Cc1ccc(F)cc1)c1ccc(NC(=O)CC(=O)OC)cc1 ; C#CCN(Cc1ccc(F)cc1)c1ccc(NC(=O)C2CCC(F)(F)CC2)cc1 ; C#CC1(N(Cc2ccc(F)cc2)c2ccc(NC(=O)OC)cc2)CC1]

### Formula 147

In an embodiment, the Kv7 channel activator may be selected from one of the following compounds according to Formula 147. Such compounds are described in International Publication No. WO2013165575A1, published Nov. 7, 2013, and corresponding to International Application No. PCT/US2013/030984 filed Mar. 13, 2013; U.S. Pat. No. 9,556,114 issued Jan. 31, 2017, and corresponding to U.S. application Ser. No. 14/566,167 filed Dec. 10, 2014; US Publication No. US20170096390A1, published Apr. 6, 2017, and corresponding to U.S. application Ser. No. 15/380,216 filed Dec. 15, 2016; which are incorporated by reference in their entirety herein. In the case of any conflict of terminology in the context of Formula 147, these references incorporated by reference herein control.

In an embodiment, the Kv7 channel activator is a compound according to Formula 147:

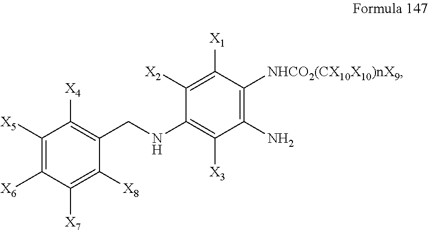

or a pharmaceutically acceptable salt or solvate thereof, wherein: X₁, X₂, X₃, X₄, X₅, X₆, X₇, and X₈ are each independently selected from hydrogen, deuterium, and F; X₉ and X₁₀ are each independently selected from hydrogen and deuterium; and n is 1, 2, or 3, wherein at least one of X₁, X₂, and X₃ is F.

In further embodiments, the Kv7 channel activator is:

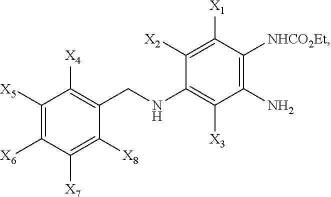

wherein, A is N or C—X₃; X₁, X₂, X₃, X₄, X₅, X₆, X₇, and X₈ are each independently selected from hydrogen, 19F and 18F; and n is 2 or 3, provided that one of X₁, X₂, X₃, X₄, X₅, X₆, X₇, and X₈ is 18F.

In further embodiments, two of X₁, X₂, X₃, X₄, X₅, X₆, X₇ and X₈ are F.

In further embodiments, three of X₁, X₂, X₃, X₄, X₅, X₆, X₇ and X₈ are F.

In further embodiments, the Kv7 channel activator is:

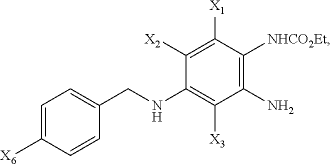

wherein, X₁, X₂, X₃, and X₆ are each independently selected from hydrogen, 18F, and 19F, provided that one of X₁, X₂, X₃, and X₆ is 18F.

In further embodiments, X₆ is fluorine and one of X₁, X₂, and X₃ is fluorine.

In further embodiments, one of X₁, X₂, X₃, and X₆ is 18F.

In further embodiments, at least one of X₁, X₂, and X₃ is 19F.

In further embodiments, X₆ is 19F or 18F.

In further embodiments, the compound is selected from the group consisting of:

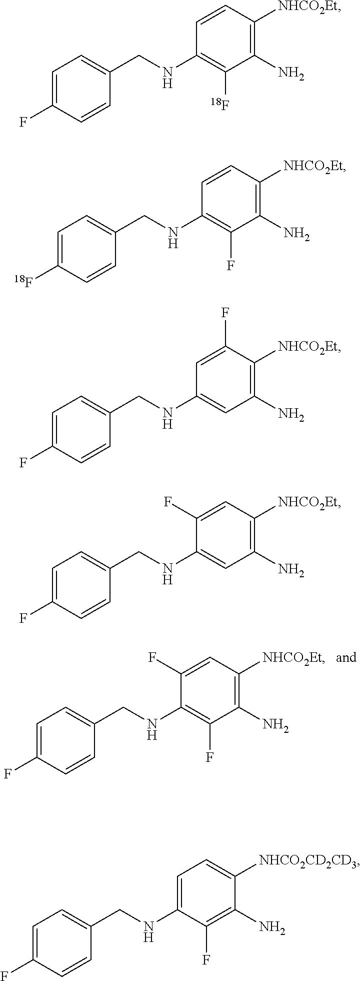

### Formula 148

In an embodiment, the Kv7 channel activator may be selected from one of the following compounds according to Formula 148. Such compounds are described in US Publication No. US20130184294A1, published Jul. 18, 2013, and corresponding to U.S. application Ser. No. 13/822,669 filed Sep. 7, 2011; International Publication No. WO2012038850A1, published Mar. 29, 2012, and corresponding to International Application No. PCT/IB2011/053917 filed Sep. 7, 2011; which are incorporated by reference in their entirety herein. In the case of any conflict of terminology in the context of Formula 148, these references incorporated by reference herein control.

In an embodiment, the Kv7 channel activator is a compound according to Formula 148:

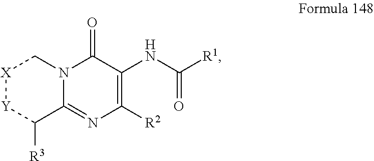

wherein, R¹ is alkyl, cycloalkyl, wherein alkyl or cycloalkyl may be substituted with one or more halogen, alkoxy, aryl, or aryloxy; R² is cycloalkyl or NR⁴R⁵; R³ is H, halogen, alkyl, or alkoxy, wherein any alkyl may be substituted with one or more halogen atoms; R⁴ and R⁵ are independently selected from H, C₁₋₆alkyl, C₃₋₆cycloalkyl, or —C₁₋₆alkyl-C₃₋₆cycloalkyl, wherein each alkyl or each cycloalkyl may be substituted with one or more halogen, provided that both R⁴ and R⁵ are not H simultaneously; or R⁴ and R⁵, together with the N atom to which they are attached, form a heterocycloalkyl; —X—Y— is ═CH—CH═, —CH₂—CH₂—, or —CH₂-; or a pharmaceutically acceptable salt thereof.

In further embodiments, R¹ is C₅₋₆ alkyl; R² is NR⁴R⁵, wherein one of R⁴ or R⁵ is H and the other is C₃₋₆alkyl, C₃₋₆cycloalkyl, or —C₁₋₃alkyl-C₃₋₆cycloalkyl; R³ is halogen, alkyl, or alkoxy, wherein any alkyl may be substituted with one or more halogen atoms; —X—Y— is ═CH—CH═ or —CH₂—CH₂-; or a pharmaceutically acceptable salt thereof.

In further embodiments, R¹ is —CH₂C(CH₃)₃; R³ is chloro, methyl, trifluoromethyl or 1,1-difluoroethoxy; R⁴ is H and R⁵ is isopropyl, isobutyl, 1-cyclopropylethyl, cyclobutyl or cyclopentyl; —X—Y— is ═CH—CH═ or —CH₂—CH₂-; or a pharmaceutically acceptable salt thereof.

In further embodiments, R¹ is C₅₋₆ alkyl; R² is C₃₋₆cycloalkyl; R³ is halogen, or alkyl, wherein alkyl may be substituted with one or more halogen atoms; —X—Y— is ═CH—CH═ or —CH₂—CH₂-; or a pharmaceutically acceptable salt thereof.

In further embodiments, R¹ is —CH₂C(CH₃)₃; R² is cyclopropyl; R³ is chloro, methyl, trifluoromethyl or 1,1-difluoroethoxy; —X—Y— is ═CH—CH═ or —CH₂—CH₂; or a pharmaceutically acceptable salt thereof.

### Formula 149

In an embodiment, the Kv7 channel activator may be selected from one of the following compounds according to Formula 149. Such compounds are described in U.S. Pat. No. 8,466,201, issued Jun. 18, 2013, and corresponding to U.S. application Ser. No. 12/746,524 filed Dec. 5, 2008; U.S. Pat. No. 9,464,052, issued Oct. 11, 2016, and corresponding to U.S. application Ser. No. 13/054,960 filed Jul. 20, 2009; U.S. Pat. No. 9,675,567 issued Jun. 13, 2017, and corresponding to U.S. application Ser. No. 15/259,064 filed Sep. 8, 2016; U.S. Pat. No. 8,466,201 issued Jun. 18, 2013, and corresponding to U.S. application Ser. No. 12/746,524 filed Dec. 5, 2008; International Publication No. WO2009071947A2, published Jun. 11, 2009, and corresponding to International Application No. PCT/GB2008/051158 filed Dec. 5, 2008; International Publication No. WO2010010380A1, published Jan. 28, 2010, and corresponding to International Application No. PCT/GB2009/050887 filed Jul. 20, 2009; which are incorporated by reference in their entirety herein. In the case of any conflict of terminology in the context of Formula 149, these references incorporated by reference herein control.

In an embodiment, the Kv7 channel activator is a compound according to Formula 149:

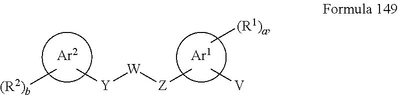

wherein, Ar¹ and Ar² are each phenyl; are the same or different and each is an aryl group or a heteroaryl group; a is an integer of from 0 to 5; R¹ is selected from the group consisting of alkyl groups, halogen atoms, haloalkyl groups, alkoxy groups, alkoxycarbonyl groups, carboxyl groups, hydroxyl groups, amino groups, monalkylamino groups, dialkylamino groups, acylamino groups, alkoxycarbonylamino groups, alkylsulphonyl groups, alkylsulphonylamino groups and cyano groups and, where a is greater than 1, each substituent R¹ may be the same or different; b is an integer of from 0 to 5; R² is selected from the group consisting of alkyl groups, halogen atoms, haloalkyl groups, alkoxy groups, alkoxycarbonyl groups, carboxyl groups, hydroxyl groups, amino groups, monalkylamino groups, dialkylamino groups, nitro groups, acylamino groups, alkoxycarbonylamino groups, alkylsulphonyl groups, alkylsulphonylamino groups and cyano groups and where b is greater than 1, each substituent R² may be the same or different; V is selected from the group consisting of (CR^{3a}R^{3b})ₚSO₂N(R³b)X; W is NR^{4a}, X is a substituent selected from the group consisting of hydroxyalkyl groups, and polyalkylene glycol residues; Y and Z are each a group of formula (CR^{5a}R^{5b})ₙ₁, wherein each n1 is 0; and p is an integer of from 0 to 2, and wherein Vis substituting said Ar¹ at an ortho position with respect to said Z or said W.

In further embodiments, a is an integer of from 0 to 3.

In further embodiments, R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different.

In further embodiments, b is an integer of from 0 to 4.

In further embodiments, R² is selected from the group consisting of alkyl groups having from 1 to 3 carbon atoms, halogen atoms, haloalkyl groups having from 1 to 3 carbon atoms, alkoxy groups having from 1 to 3 carbon atoms, carboxyl groups, amino groups, hydroxyl groups and cyano groups.

In further embodiments, R^{4a} is selected from the group consisting of hydrogen atoms, alkyl groups having from 1 to 3 carbon atoms and phenyl groups.

In further embodiments, V is selected from the group consisting of (CR^{3a}R^{3b})ₚSO₂N(R^{3b})X, wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, and polyalkylene glycol residues of general formula HO—[(CR(CR^{6a}R^{6b})_{c1}—O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms.

In further embodiments, V is a group of formula of (CR^{3a}R^{3b})ₚSO₂N(R^{3b})X, wherein p is an integer of 0 or 1, R^{3a} and R^{3b} are the same or different and each is selected from the group consisting of hydrogen atoms and alkyl groups having from 1 to 4 carbon atoms, and X is a substituent selected from the group consisting of hydroxyalkyl groups having from 1 to 4 carbon atoms and polyalkylene glycol residues of general formula HO—[(CR^{6a}R^{6b})_{c1}—O—(CR^{6c}R^{6d})_{c2}]_{c3}— wherein c1 and c2 are the same or different and each is an integer of from 1 to 3, c3 is an integer of from 1 to 10 and R^{6a}, R^{6b}, R^{6c}, R^{6d} may be the same or different and each is a hydrogen atom or an alkyl group having from 1 to 4 carbon atoms.

In further embodiments, V is a group of formula of (CR^{3a}R^{3b})ₚSO₂N(R^{3b})X, wherein p is an integer of 0 or 1, each of R^{3a} and R^{3b} is a hydrogen atom, and X is a hydroxylalkyl group having from 1 to 4 carbon atoms or a polyalkylene glycol residue of general formula HO—[CR^{6a}R^{6b})_{c1}—O—(CR^{6c}R^{6d})_{c2}]_{c3}— wherein c1 and c2 are the same or different and each is 1 or 2, c3 is an integer of from 1 to 6 and each of R^{6a}, R^{6b}, R^{6c} and R^{6d} is a hydrogen atom.

In further embodiments, Ar¹ and Ar² are each phenyl; a is an integer of from 0 to 3; R¹ is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where a is greater than 1, each substituent R¹ may be the same or different; b is an integer of from 0 to 4; R² is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, nitro groups, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where b is greater than 1, each substituent R² may be the same or different; W is NR^{4a}, wherein R^{4a} is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms; Y and Z are each a group of formula (CR^{5a}R^{5b})ₙ₁, wherein each n1 is 0; and V is selected from the group consisting of (CR^{3a}R^{3b})ₚSO₂N(R^{3b})X and, wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, carbonyl groups, hydroxyl groups and cyano groups, and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, and polyalkylene glycol residues of general formula HO—[(CR^{6a}R^{6b})_{c1}—O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms.

In further embodiments, Ar1 and Ar2 are each phenyl; a is 0 or 1; R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different; b is an integer of from 0 to 3; R² is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, nitro groups, acylamino groups having from 1 to 4 carbon atoms, alkylsulphonyl groups having from 1 to 4 carbon atoms, alkylsulphonylamino groups having from 1 to 4 carbon atoms and cyano groups and, where b is greater than 1, each substituent R² may be the same or different; W is NR^{4a}, wherein R^{4a} is selected from the group consisting of hydrogen atoms, alkyl groups having from 1 to 4 carbon atoms, aryl groups having from 6 to 10 carbon atoms and 5- or 6-membered aromatic heterocyclic groups containing 1 or 2 sulfur atoms, oxygen atoms and/or nitrogen atoms; Y and Z are each a group of formula (CR^{5a}R^{5b})ₙ₁, wherein each n1 is 0; and V is a group of formula of (CR^{3a}R^{3b})ₚSO₂N(R^{3b})X, wherein p is an integer of 0 or 1, R^{3a} and R^{3b} are the same or different and each is selected from the group consisting of hydrogen atoms and alkyl groups having from 1 to 4 carbon atoms, and X is a substituent selected from the group consisting of hydroxyalkyl groups having from 1 to 4 carbon atoms and polyalkylene glycol residues of general formula HO—[(CR^{6a}R^{6b})_{c1}—O— (CR^{6c}R^{6d})_{c2}]_{c3}— wherein c1 and c2 are the same or different and each is an integer of from 1 to 3, c3 is an integer of from 1 to 10 and R^{6a}, R^{6b}, R^{6c} and R^{6d} may be the same or different and each is a hydrogen atom or an alkyl group having from 1 to 4 carbon atoms.

In further embodiments, Ar1 and Ar2 are each phenyl; a is 0 or 1; R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different; b is 0 to 3; R² is selected from the group consisting of alkyl groups having from 1 to 3 carbon atoms, halogen atoms, haloalkyl groups having from 1 to 3 carbon atoms, alkoxy groups having from 1 to 3 carbon atoms, carboxyl groups, amino groups, hydroxyl groups and cyano groups; W is a group of formula NR^{4a}, wherein R^{4a} is selected from the group consisting of hydrogen atoms, alkyl groups having from 1 to 3 carbon atoms and phenyl groups; Y and Z are each a group of formula (CR^{5a}R^{5b})ₙ₁ wherein each n1 is 0; and Vis a group of formula of (CR^{3a}R^{3b})ₚSO₂N(R^{3b})X, wherein p is an integer of 0 or 1, each of R^{3a} and R^{3b} is a hydrogen atom, and X is a hydroxylalkyl group having from 1 to 4 carbon atoms or a polyalkylene glycol residue of general formula HO—[(CR^{6a}R^{6b})_{c1}—O—(CR^{6c}R^{6d})_{c2}]_{c3}— wherein c1 and c2 are the same or different and each is 1 or 2, c3 is an integer of from 1 to 6 and each of R^{6a}, R^{6b}, R^{6c} and R^{6d} is a hydrogen atom.

In further embodiments, Ar¹ and Ar² are each phenyl; a is an integer of from 0 to 3; R¹ is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where a is greater than 1, each substituent R¹ may be the same or different; b is an integer of from 0 to 4; R² is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, nitro groups, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where b is greater than 1, each substituent R² may be the same or different; W is NR^{4a} wherein R^{4a} is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms; and V is selected from the group consisting of (CR^{3a}R^{3b})ₚSO₂N(R^{3b})X wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, and polyalkylene glycol residues of general formula HO—[(CR^{6a}R^{6b})_{c1}—O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms.

In further embodiments, the Kv7 Channel activator is selected from the group consisting of: 1-[2-(2,6-Dichlorophenylamino)phenyl]-N-[2-(2-hydroxyethoxy)ethyl]-methanesulfonamide, N-[2-(2-hydroxyethoxy)ethyl]-C-[2-(2,4,6-trichlorophenylamino)phenyl]-methanesulfonamide, 1-[[2-(2,6-dichloro-4-trifluoromethylphenylamino)phenyl]-N-{2-(2-hydroxyethoxy)ethyl]methanesulfonamide, 1-[[2-(2,6-dichloro-3-methylphenylamino)phenyl]-N-{2-(2-hydroxyethoxy)-ethyl]methanesulfonamide, N-(2-hydroxyethyl)-1-[3-(2,4,6-trichlorophenylamino)phenyl]methane sulphonamide, N-(2-hydroxyethyl)-2-(2,4,6-trichlorophenylamino)benzenesulfonamide, N-(2-hydroxyethyl)-2-(2,6-dichlorophenylamino)benzene sulfonamide, 2-(2,6-dichloro-4-trifluoromethylphenylamino)-N-(2-hydroxyethyl)-benzenesulfonamide, 2-(2,6-dichloro-3-methylphenylamino)-N-(2-hydroxyethyl)-benzenesulfonamide, 2-(2,6-dichloro-4-trifluoromethylphenylamino)-N-[2-(2-hydroxyethoxy)ethyl]-benzenesulfonamide, 2-(2,6-dichloro-3-methylphenylamino)-N-[2-(2-hydroxyethoxy)ethyl]-benzenesulfonamide, 2-(3,5-dichlorophenylamino)-N-[2-(2-hydroxyethoxy)ethyl]-benzenesulfonamide, and N-[2-(2-hydroxyethoxy)ethyl]-2-(2,4,6-trichlorophenylamino)-benzenesulfonamide, or a pharmacologically acceptable salt or pro-drug thereof.

In further embodiments, the compound is according to Formula 149 wherein: Ar1 and Ar2 are the same or different and each is an aryl group or a heteroaryl group; a is an integer of from 0 to 5; R¹ is selected from the group consisting of alkyl groups, halogen atoms, haloalkyl groups, alkoxy groups, haloalkoxy groups, alkoxycarbonyl groups, carboxyl groups, hydroxyl groups, amino groups, monoalkylamino groups, dialkylamino groups, nitro groups, acylamino groups, alkoxycarbonylamino groups, alkylsulphonyl groups, alkylsulphonylamino groups and cyano groups and, where a is greater than 1, each substituent R¹ may be the same or different; b is an integer of from 0 to 5; R² is selected from the group consisting of alkyl groups, halogen atoms, haloalkyl groups, haloalkoxy groups, alkoxy groups, alkoxycarbonyl groups, carboxyl groups, hydroxyl groups, amino groups, monoalkylamino groups, dialkylamino groups, nitro groups, acylamino groups, alkoxycarbonylamino groups, alkylsulphonyl groups, alkylsulphonylamino groups and cyano groups and where b is greater than 1, each substituent R² may be the same or different; Vis selected from the group consisting of (CR^{3a}R^{3b})ₚCON(R^{3b})X and (CR^{3a}R^{3b})ₚN(R^{3b})CO(X), wherein said groups are in the 3-(meta) or 4-(para) position with respect to the substituent Z; W is selected from the group consisting of NR^{4a}, O, S, S═O, SO₂ and C(R^{4a}R^{4b})₂; X is a substituent selected from the group consisting of hydroxyalkyl groups, alkoxyalkyl groups, haloalkoxyalkyl groups, aryloxyalkyl groups, polyalkylene glycol residues, carboxyalkyl groups, alkoxycarbonylalkyl groups, haloalkoxycarbonylalkyl groups and aralkyloxycarbonylalkyl groups; Y and Z are the same or different and each is a substituent selected from the group consisting of (CR^{5a}R^{5b})ₙ₁, C═O, SO₂, C(═O)NR^{5a}, C(═O)NR^{5a}SO₂ and CO(R^{5a}R^{5b})ₙ₂; R^{3a}, R^{3b}, R^{4a}, R^{4b}, R^{5a} and R^{5b} are the same or different and each is selected from the group consisting of hydrogen atoms, alkyl groups, cycloalkyl groups, aryl groups and heteroaryl groups; n1 and n2 are the same or different and each is an integer of from 0 to 2; and p is an integer of from 0 to 2.

In further embodiments, Ar¹ and Ar² are the same or different and each is an aryl group having from 5 to 14 carbon atoms or a 5- to 7-membered aromatic heterocyclic group containing from 1 to 3 sulfur atoms, oxygen atoms and/or nitrogen atoms.

In further embodiments, Ar¹ and Ar² are the same or different and each is an aryl group having from 6 to 10 carbon atoms or a 5- or 6-membered aromatic heterocyclic group containing 1 or 2 sulfur atoms, oxygen atoms and/or nitrogen atoms.

In further embodiments, Ar¹ and Ar² are each phenyl.

In further embodiments, R² is selected from the group consisting of alkyl groups having from 1 to 3 carbon atoms, halogen atoms, haloalkyl groups having from 1 to 3 carbon atoms, haloalkoxy groups having from 1 to 3 carbon atoms, alkoxy groups having from 1 to 3 carbon atoms, carboxyl groups, amino groups, hydroxyl groups and cyano groups.

In further embodiments, W is a group of formula NR^{4a}, wherein R^{4a} is selected from the group consisting of hydrogen atoms, alkyl groups having from 1 to 3 carbon atoms and phenyl groups.

In further embodiments, Y and Z are each a group of formula (CR^{5a}R^{5b})ₙ₁ wherein each n1 is 0.

In further embodiments, V is selected from the group consisting of (CR^{3a}R^{3b})ₚCON(R^{3b})X and (CR^{3a}R^{3b})ₚN(R^{3b}CO(X), wherein said groups are in the 3-(meta) or 4-(para) position with respect to the substituent Z, wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, alkoxyalkyl groups comprising alkyl groups having from 1 to 6 carbon atoms which are substituted by at least one alkoxy group having from 1 to 6 carbon atoms, haloalkoxyalkyl groups comprising alkyl groups having from 1 to 6 carbon atoms which are substituted by at least one haloalkoxy group having from 1 to 6 carbon atoms, aryloxy groups having from 5 to 14 carbon atoms, polyalkylene glycol residues of general formula HO—[(CR^{6a}R^{6b})_{c1}—O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms, carboxyalkyl groups comprising alkyl groups having from 1 to 6 carbon atoms which are substituted with at least one carboxy group, alkoxycarbonylalkyl groups comprising alkyl groups having from 1 to 6 carbon atoms which are substituted with at least one alkoxycarbonyl group having from 2 to 7 carbon atoms, haloalkoxycarbonylalkyl groups comprising carbonylalkyl groups having from 2 to 7 carbon atoms that are substituted with haloalkoxy groups having from 1 to 6 carbon atoms and aralkyloxycarbonylalkyl groups comprising carbonylalkyl groups having from 2 to 7 carbon atoms which are substituted with at least one aralkyloxy group that comprises an alkyl group having from 1 to 6 carbon atoms that is substituted with an aryl group having from 5 to 14 carbon atoms.

In further embodiments, V is selected from the group consisting of (CR^{3a}R^{3b})ₚCON(R^{3b})X and (CR^{3a}R^{3b})ₚN(R^{3b})CO(X), wherein said groups are in the 3-(meta) or 4-(para) position with respect to the substituent Z, wherein p is an integer of 0 or 1, R^{3a} and R^{3b} are the same or different and each is selected from the group consisting of hydrogen atoms and alkyl groups having from 1 to 4 carbon atoms, and X is a substituent selected from the group consisting of hydroxyalkyl groups having from 1 to 4 carbon atoms, polyalkylene glycol residues of general formula HO—[(CR^{6a}R^{6b})_{c1}—O—(CR^{6c}R^{6d})_{c2}]_{c3}— wherein c1 and c2 are the same or different and each is an integer of from 1 to 3, c3 is an integer of from 1 to 10 and R^{6a}, R^{6b}, R^{6c} and R^{6d} may be the same or different and each is a hydrogen atom or an alkyl group having from 1 to 4 carbon atoms, alkoxyalkyl groups comprising an alkyl group having from 1 to 4 carbon atoms that is substituted with at least one alkoxy group having from 1 to 4 carbon atoms, carboxyalkyl groups comprising an alkyl group having from 1 to 4 carbon atoms that is substituted with at least one carboxy group, carboxyalkyl groups comprising alkyl groups having from 1 to 4 carbon atoms which are substituted with at least one carboxy group and alkoxycarbonylalkyl groups comprising alkyl groups having from 1 to 4 carbon atoms which are substituted with at least one alkoxycarbonyl group having from 2 to 5 carbon atoms.

In further embodiments, V is selected from the group consisting of (CR^{3a}R^{3b})ₚCON(R^{3b})X and (CR^{3a}R^{3b})ₚN(R^{3b})CO(X), wherein said groups are in the 3-(meta) or 4-(para) position with respect to the substituent Z, wherein p is an integer of 0 or 1, each of R^{3a} and R^{3b} is a hydrogen atom, and X is selected from the group consisting of alkyl groups having 1 or 2 carbon atoms which are substituted with a group selected from hydroxyl groups, methoxy groups, ethoxy groups, carboxy groups, methoxycarbonyl groups and ethoxycarbonyl groups.

In further embodiments, the compound is according to Formula 149 wherein: Ar¹ and Ar² are each phenyl; a is 0 or 1; R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, haloalkoxy groups having from 1 to 4 carbon atoms, carboxyl groups, hydroxyl groups, amino groups, monoalkylamino groups wherein the alkyl group has from 1 to 4 carbon atoms, dialkylamino groups wherein the alkyl groups may be the same or different and each has from 1 to 4 carbon atoms, nitro groups, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different; b is 0 to 3; R² is selected from the group consisting of alkyl groups having from 1 to 3 carbon atoms, halogen atoms, haloalkyl groups having from 1 to 3 carbon atoms, alkoxy groups having from 1 to 3 carbon atoms, haloalkoxy groups having from 1 to 3 carbon atoms, carboxyl groups, amino groups, hydroxyl groups and cyano groups; W is a group of formula NR^{4a}, wherein R^{4a} is selected from the group consisting of hydrogen atoms, alkyl groups having from 1 to 3 carbon atoms and phenyl groups; Y and Z are each a group of formula (CR^{5a}R^{5b})ₙ₁ wherein each n1 is 0; and V is selected from the group consisting of (CR^{3a}R^{3b})ₚCON(R^{3b})X and (CR^{3a}R^{3b})ₚN(R^{3b})CO(X), wherein said groups are in the 3-(meta) or 4-(para) position with respect to the substituent Z, wherein p is an integer of 0 or 1, each of R^{3a} and R^{3b} is a hydrogen atom, and X is selected from the group consisting of alkyl groups having 1 or 2 carbon atoms which are substituted with a group selected from hydroxyl groups, methoxy groups, ethoxy groups, carboxy groups, methoxycarbonyl groups and ethoxycarbonyl groups.

In further embodiments, the Kv7 Channel activator is selected from the group consisting of: N-(2-hydroxyethyl)-2-[3-(2,4,6-trichlorophenylamino)phenyl]acetamide; {2-[5-(3,5-dichlorophenylamino)-2-fluorophenyl]-acetylamino}acetic acid; 2-[4-(2,6-dichloro-4-trifluoromethyl-phenylamino)-phenyl]-N-(2-hydroxyethyl)-acetamide; {2-[4-(3,5-dichlorophenylamino)-phenyl]-acetylamino}acetic acid; N-(2-hydroxy-ethyl)-2-[4-(2,4,6-trichloro-phenylamino)-phenyl]acetamide; 3-(2,6-dichloro-4-trifluoromethyl-phenylamino)-N-(2-hydroxy-ethyl)benzamide; 2-[2-fluoro-5-(2,4,6-trichloro-phenylamino)-phenyl]-N-(2-hydroxy-ethyl) acetamide; 2-[5-(2,6-dichloro-4-trifluoromethyl-phenylamino)-2-fluoro-phenyl]-N-(2-hydroxy-ethyl) acetamide; 2-[5-(2,6-dichloro-4-trifluoromethoxy-phenylamino)-2-fluoro-phenyl]-N-(2-hydroxy-ethyl) acetamide; 2-[4-(2,6-dichloro-4-trifluoromethoxy-phenylamino)-phenyl]-N-(2-hydroxy-ethyl)-acetamide; 5-(2,6-dichloro-4-trifluoromethyl-phenylamino)-2-fluoro-N-(2-hydroxy-ethyl)-benzamide; 2-[4-(2,6-dichloro-4-trifluoromethyl-phenylamino)-phenyl]-N-(2-hydroxy-2-methyl-propyl) acetamide; 2-[5-(2,6-dichloro-4-trifluoromethyl-phenylamino)-2-fluoro-phenyl]-N-(2-hydroxy-2-methyl-propyl) acetamide; and 2-[4-(2,6-dichloro-4-trifluoromethoxy-phenylamino)-2-fluoro-phenyl]-N-(2-hydroxy-ethyl)propionamide.

In further embodiments, the compound is according to formula 150 wherein: Ar¹ and Ar² are the same or different and each is an aryl group or a heteroaryl group; a is an integer of from 0 to 5; R¹ is selected from the group consisting of alkyl groups, halogen atoms, haloalkyl groups, alkoxy groups, haloalkoxy groups, alkoxycarbonyl groups, carboxyl groups, hydroxyl groups, amino groups, monoalkylamino groups, dialkylamino groups, nitro groups, acylamino groups, alkoxycarbonylamino groups, alkylsulphonyl groups, alkylsulphonylamino groups and cyano groups and, where a is greater than 1, each substituent R¹ may be the same or different; b is an integer of from 0 to 5; R² is selected from the group consisting of alkyl groups, halogen atoms, haloalkyl groups, haloalkoxy groups, alkoxy groups, alkoxycarbonyl groups, carboxyl groups, hydroxyl groups, amino groups, monoalkylamino groups, dialkylamino groups, nitro groups, acylamino groups, alkoxycarbonylamino groups, alkylsulphonyl groups, alkylsulphonylamino groups and cyano groups and where b is greater than 1, each substituent R² may be the same or different; Vis selected from the group consisting of (CR^{3a}R^{3b})ₚCON(R^{3b})X and (CR^{3a}R^{3b})ₚN(R^{3b})CO(X), wherein said groups are in the 3-(meta) or 4-(para) position with respect to the substituent Z; W is selected from the group consisting of NR^{4a}, O, S, S═O, SO₂ and C(R^{4a}R^{4b})₂; X is a substituent selected from the group consisting of hydrogen atoms, alkyl groups, hydroxyalkyl groups, alkoxyalkyl groups, haloalkoxyalkyl groups, aryloxyalkyl groups, cycloalkyl groups, aryl groups, heteroaryl groups, polyalkylene glycol residues, aminoalkyl groups, monoalkylaminoalkyl groups, dialkylaminoalkyl groups; alkyl groups that are substituted with groups of formula NR8R9 wherein R8 and R9 together with the nitrogen atom to which they are attached form a saturated or partially unsaturated heterocyclic group which optionally contains at least one more heteroatom selected from nitrogen, oxygen and sulphur atoms (said saturated or partially unsaturated heterocyclic group optionally further being substituted by one or more substituents selected from the group consisting of alkyl groups, halogen atoms, haloalkyl groups, alkoxy groups, alkoxycarbonyl groups, carboxyl groups, nitro groups, amino groups, monoalkylamino groups, dialkylamino groups and hydroxyl groups), carboxyalkyl groups, alkoxycarbonylalkyl groups, haloalkoxycarbonylalkyl groups and aralkyloxycarbonylalkyl groups; Y and Z are the same or different and each is a substituent selected from the group consisting of (CR^{5a}R^{5b})ₙ₁, C═O, SO₂, C(═O)NR^{5a}, C(═O)NR^{5a}SO₂ and C═O(R^{5a}R^{5b})ₙ₂; R^{3a}, R^{3b}, R^{4a}, R^{4b}, R^{5a} and R^{5b} are the same or different and each is selected from the group consisting of hydrogen atoms, alkyl groups, cycloalkyl groups, aryl groups and heteroaryl groups; n1 and n2 are the same or different and each is an integer of from 0 to 2; and p is an integer of from 0 to 2.

In further embodiments, the compound is according to Formula 149 wherein: Ar¹ and Ar² are each phenyl; a is 1; R¹ is halogen; b is an integer of from 0 to 3; R² is selected from the group consisting of alkyl having from 1 to 3 carbon atoms, halogen, haloalkyl having from 1 to 3 carbon atoms, alkoxy having from 1 to 3 carbon atoms, haloalkoxy having from 1 to 3 carbon atoms, carboxyl, amino, hydroxyl and cyano, and where b is greater than 1, each substituent R² may be the same or different; Vis (CR^{3a}R^{3b})ₚCON(R^{3b})X, wherein said group is in the 3-(meta) or 4-(para) position with respect to the substituent Z; W is NR^{4a}, wherein R^{4a} is selected from the group consisting of hydrogen, alkyl having from 1 to 3 carbon atoms and phenyl; X is a substituent selected from the group consisting of hydroxyalkyl having from 1 to 4 carbon atoms, polyalkylene glycol residue of general formula: HO—[(CR^{6a}R^{6b})_{c1}—O—(CR^{6c}R^{6d})_{c2}]_{c3}— wherein c1 and c2 are the same or different and each is an integer of from 1 to 3, c3 is an integer of from 1 to 10 and R^{6a}, R^{6b}, R^{6c} and R^{6d} may be the same or different and each is a hydrogen or an alkyl having from 1 to 4 carbon atoms, alkoxyalkyl comprising an alkyl having from 1 to 4 carbon atoms that is substituted with at least one alkoxy having from 1 to 4 carbon atoms, carboxyalkyl comprising an alkyl having from 1 to 4 carbon atoms that is substituted with at least one carboxy, carboxyalkyl comprising alkyl having from 1 to 4 carbon atoms which are substituted with at least one carboxy and alkoxycarbonylalkyl comprising alkyl having from 1 to 4 carbon atoms which are substituted with at least one alkoxycarbonyl having from 2 to 5 carbon atoms; Y and Z are each a group of formula (CR^{5a}R^{5b})ₙ₁ wherein each n1 is 0; R^{3a} and R^{3b} are the same or different and each is selected from the group consisting of hydrogen and alkyl; and p is an integer of from 0 to 2.

In further embodiments, Vis (CR^{3a}R^{3b})ₚCON(R^{3b})X, wherein said groups is in the 3-(meta) or 4-(para) position with respect to the substituent Z, wherein p is an integer of 0 or 1, each of R^{3a} and R^{3b} is hydrogen, and X is selected from the group consisting of alkyl having 1 or 2 carbon atoms which are substituted with a group selected from hydroxyl groups, methoxy groups, ethoxy groups, carboxy groups, methoxycarbonyl groups and ethoxycarbonyl groups.

In further embodiments, the Kv7 Channel activator is selected from the group consisting of: {2-[5-(3,5-dichlorophenylamino)-2-fluorophenyl]-acetylamino}acetic acid; 2-[5-(2,6-dichloro-4-trifluoromethoxy-phenylamino)-2-fluoro-phenyl]-N-(2-hydroxy-ethyl) acetamide; 5-(2,6-dichloro-4-trifluoromethyl-phenylamino)-2-fluoro-N-(2-hydroxy-ethyl)-benzamide; 2-[5-(2,6-dichloro-4-trifluoromethyl-phenylamino)-2-fluoro-phenyl]-N-(2-hydroxy-2-methyl-propyl) acetamide; and 2-[4-(2,6-dichloro-4-trifluoromethoxy-phenylamino)-2-fluoro-phenyl]-N-(2-hydroxy-ethyl)propionamide.

In further embodiments, the compound is according to Formula 149 wherein: Ar¹ and Ar² are each phenyl; are the same or different and each is an aryl group or a heteroaryl group; a is an integer of from 0 to 5; R¹ is selected from the group consisting of alkyl groups, halogen atoms, haloalkyl groups, alkoxy groups, alkoxycarbonyl groups, carboxyl groups, hydroxyl groups, amino groups, monalkylamino groups, dialkylamino groups, acylamino groups, alkoxycarbonylamino groups, alkylsulphonyl groups, alkylsulphonylamino groups and cyano groups and, where a is greater than 1, each substituent R¹ may be the same or different; b is an integer of from 0 to 5; R² is selected from the group consisting of alkyl groups, halogen atoms, haloalkyl groups, alkoxy groups, alkoxycarbonyl groups, carboxyl groups, hydroxyl groups, amino groups, monalkylamino groups, dialkylamino groups, nitro groups, acylamino groups, alkoxycarbonylamino groups, alkylsulphonyl groups, alkylsulphonylamino groups and cyano groups and where b is greater than 1, each substituent R² may be the same or different; V is selected from the group consisting of (CR^{3a}R^{3b})ₚSO₂N(R³b)X; W is NR^{4a}, X is a substituent selected from the group consisting of hydroxyalkyl groups, and polyalkylene glycol residues; Y and Z are each a group of formula (CR^{5a}R^{5b})ₙ₁, wherein each n1 is 0; and p is an integer of from 0 to 2, and wherein Vis substituting said Ar¹ at an ortho position with respect to said Z or said W. In further embodiments,

In further embodiments, a is an integer of from 0 to 3.

In further embodiments, R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different.

In further embodiments, b is an integer of from 0 to 4.

In further embodiments, R² is selected from the group consisting of alkyl groups having from 1 to 3 carbon atoms, halogen atoms, haloalkyl groups having from 1 to 3 carbon atoms, alkoxy groups having from 1 to 3 carbon atoms, carboxyl groups, amino groups, hydroxyl groups and cyano groups.

In further embodiments, R^{4a} is selected from the group consisting of hydrogen atoms, alkyl groups having from 1 to 3 carbon atoms and phenyl groups.

In further embodiments, V is selected from the group consisting of (CR^{3a}R^{3b})ₚSO₂N(R^{3b})X, wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, and polyalkylene glycol residues of general formula HO—[(CR(CR^{6a}R^{6b})_{c1}—O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms.

In further embodiments, V is a group of formula of (CR^{3a}R^{3b})ₚSO₂N(R^{3b})X, wherein p is an integer of 0 or 1, R^{3a} and R^{3b} are the same or different and each is selected from the group consisting of hydrogen atoms and alkyl groups having from 1 to 4 carbon atoms, and X is a substituent selected from the group consisting of hydroxyalkyl groups having from 1 to 4 carbon atoms and polyalkylene glycol residues of general formula HO—[(CR^{6a}R^{6b})_{c1}—O—(CR^{6c}R^{6d})_{c2}]_{c3}— wherein c1 and c2 are the same or different and each is an integer of from 1 to 3, c3 is an integer of from 1 to 10 and R^{6a}, R^{6b}, R^{6c}, R^{6d} may be the same or different and each is a hydrogen atom or an alkyl group having from 1 to 4 carbon atoms.

In further embodiments, V is a group of formula of (CR^{3a}R^{3b})ₚSO₂N(R^{3b})X, wherein p is an integer of 0 or 1, each of R^{3a} and R^{3b} is a hydrogen atom, and X is a hydroxylalkyl group having from 1 to 4 carbon atoms or a polyalkylene glycol residue of general formula HO—[CR^{6a}R^{6b})_{c1}—O—(CR^{6c}R^{6d})_{c2}]_{c3}— wherein c1 and c2 are the same or different and each is 1 or 2, c3 is an integer of from 1 to 6 and each of R^{6a}, R^{6b}, R^{6c} and R^{6d} is a hydrogen atom.

In further embodiments, the compound is according to Formula 149 wherein: Ar¹ and Ar² are each phenyl; a is an integer of from 0 to 3; R¹ is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where a is greater than 1, each substituent R¹ may be the same or different; b is an integer of from 0 to 4; R² is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, nitro groups, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where b is greater than 1, each substituent R² may be the same or different; W is NR^{4a}, wherein R^{4a} is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms; Y and Z are each a group of formula (CR^{5a}R^{5b})ₙ₁, wherein each n1 is 0; and Vis selected from the group consisting of (CR^{3a}R^{3b})ₚSO₂N(R^{3b})X and, wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms which may optionally be substituted with at least one substituent selected from alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, carbonyl groups, hydroxyl groups and cyano groups, and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, and polyalkylene glycol residues of general formula HO—[(CR^{6a}R^{6b}): 1-O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms.

In further embodiments, the compound is according to Formula 149 wherein: Ar¹ and Ar² are each phenyl; a is 0 or 1; R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different; b is an integer of from 0 to 3; R² is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, nitro groups, acylamino groups having from 1 to 4 carbon atoms, alkylsulphonyl groups having from 1 to 4 carbon atoms, alkylsulphonylamino groups having from 1 to 4 carbon atoms and cyano groups and, where b is greater than 1, each substituent R² may be the same or different; W is NR^{4a}, wherein R^{4a} is selected from the group consisting of hydrogen atoms, alkyl groups having from 1 to 4 carbon atoms, aryl groups having from 6 to 10 carbon atoms and 5- or 6-membered aromatic heterocyclic groups containing 1 or 2 sulfur atoms, oxygen atoms and/or nitrogen atoms; Y and Z are each a group of formula (CR^{5a}R^{5b})ₙ₁, wherein each n1 is 0; and Vis a group of formula of (CR^{3a}R^{3b})ₚSO₂N(R^{3b})X, wherein p is an integer of 0 or 1, R^{3a} and R^{3b} are the same or different and each is selected from the group consisting of hydrogen atoms and alkyl groups having from 1 to 4 carbon atoms, and X is a substituent selected from the group consisting of hydroxyalkyl groups having from 1 to 4 carbon atoms and polyalkylene glycol residues of general formula HO—[(CR^{6a}R^{6b}): 1-O—(CR^{6c}R^{6d})_{c2}]_{c3}— wherein c1 and c2 are the same or different and each is an integer of from 1 to 3, c3 is an integer of from 1 to 10 and R^{6a}, R^{6b}, R^{6c} and R^{6d} may be the same or different and each is a hydrogen atom or an alkyl group having from 1 to 4 carbon atoms.

In further embodiments, the compound is according to Formula 149 wherein: Ar¹ and Ar² are each phenyl; a is 0 or 1; R¹ is selected from the group consisting of alkyl groups having from 1 to 4 carbon atoms, halogen atoms, haloalkyl groups having from 1 to 4 carbon atoms, alkoxy groups having from 1 to 4 carbon atoms, carboxyl groups, hydroxyl groups, amino groups, monalkylamino groups wherein the alkyl group has from 1 to 4 carbon atoms, dialkylamino groups wherein the alkyl groups may be the same or different and each has from 1 to 4 carbon atoms, acylamino groups having from 1 to 4 carbon atoms, alkylsulphonyl groups having from 1 to 4 carbon atoms, alkylsulphonylamino groups having from 1 to 4 carbon atoms and cyano groups and, where a is greater than 1, each substituent R¹ may be the same or different; b is 0 to 3; R² is selected from the group consisting of alkyl groups having from 1 to 3 carbon atoms, halogen atoms, haloalkyl groups having from 1 to 3 carbon atoms, alkoxy groups having from 1 to 3 carbon atoms, carboxyl groups, amino groups, hydroxyl groups and cyano groups; W is a group of formula NR^{4a}, wherein R^{4a} is selected from the group consisting of hydrogen atoms, alkyl groups having from 1 to 3 carbon atoms and phenyl groups; Y and Z are each a group of formula (CR^{5a}R^{5b})ₙ₁ wherein each n1 is 0; and V is a group of formula of (CR^{3a}R^{3b})ₚSO₂N(R^{3b})X, wherein p is an integer of 0 or 1, each of R^{3a} and R^{3b} is a hydrogen atom, and X is a hydroxylalkyl group having from 1 to 4 carbon atoms or a polyalkylene glycol residue of general formula HO—[(CR^{6a}R^{6b})_{c1}—O—(CR^{6c}R^{6d})_{c2}]_{c3}— wherein c1 and c2 are the same or different and each is 1 or 2, c3 is an integer of from 1 to 6 and each of R^{6a}, R^{6b}, R^{6c} and R^{6d} is a hydrogen atom.

In further embodiments, the compound is according to Formula 149 wherein: Ar¹ and Ar² are each phenyl; a is an integer of from 0 to 3; R¹ is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where a is greater than 1, each substituent R¹ may be the same or different; b is an integer of from 0 to 4; R² is selected from the group consisting of alkyl groups having from 1 to 6 carbon atoms, halogen atoms, haloalkyl groups having from 1 to 6 carbon atoms, alkoxy groups having from 1 to 6 carbon atoms, alkoxycarbonyl groups wherein the alkoxy group has from 1 to 6 carbon atoms, carboxyl groups, hydroxyl groups, amino groups, monalkylamino groups wherein the alkyl group has from 1 to 6 carbon atoms, dialkylamino groups wherein the alkyl groups may be the same or different and each has from 1 to 6 carbon atoms, nitro groups, acylamino groups having from 1 to 6 carbon atoms, alkoxycarbonylamino groups wherein the alkoxy group has from 1 to 6 carbon atoms, alkylsulphonyl groups having from 1 to 6 carbon atoms, alkylsulphonylamino groups having from 1 to 6 carbon atoms and cyano groups and, where b is greater than 1, each substituent R² may be the same or different; W is NR^{4a}, wherein R^{4a} is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms; and V is selected from the group consisting of (CR^{3a}R^{3b})ₚSO₂N(R^{3b})X wherein p is an integer of from 0 to 2, R^{3a} and R^{3b} are the same or different and each is selected from the group consisting of hydrogen atoms, alkyl groups having from 1 to 6 carbon atoms, cycloalkyl groups having from 3 to 7 carbon atoms, aryl groups having from 5 to 14 carbon atoms and 5- to 7-membered aromatic heterocyclic groups containing from 1 to 3 sulfur atoms, oxygen atoms and/or nitrogen atoms, and X is a substituent selected from the group consisting of hydroxyalkyl groups having from 1 to 6 carbon atoms, and polyalkylene glycol residues of general formula HO—[(CR^{6a}R^{6c})_{c1}—O—(CR^{6c}R^{6d})_{c2}]_{c3}— wherein c1 and c2 are the same or different and each is an integer of from 0 to 4, c3 is an integer of from 1 to 20 and R^{6a}, R^{6b}, R^{6c} and R^{6d} may be the same or different from each other and each is a hydrogen atom or an alkyl group having from 1 to 6 carbon atoms.

### Formula 150

In an embodiment, the Kv7 channel activator may be selected from one of the following compounds according to Formula 150. Such compounds are described in International Publication No. WO2012004698A1, published Jan. 12, 2012, and corresponding to International Application No. PCT/IB2011/052686 filed Jun. 20, 2011; U.S. Pat. No. 8,883,812, issued Nov. 11, 2014 and corresponding to U.S. application Ser. No. 13/808,355 filed Jun. 20, 2011; which are incorporated by reference in their entirety herein. In the case of any conflict of terminology in the context of Formula 150, these references incorporated by reference herein control.

In an embodiment, the Kv7 channel activator is a compound according to Formula 150:

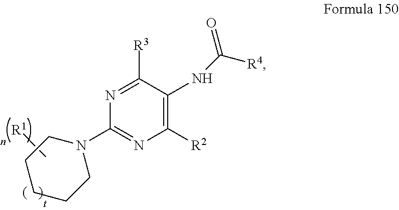

wherein, n is an integer of 1 or 2; t is 0 or 1; each R¹ is independently selected from C₁₋₃ alkoxy, C₁₋₃ alkyl, C₁₋₃ alkyl-O—C₁₋₃ alkyl; R² and R³ are independently C₁₋₃ alkyl, C₁₋₃ alkoxy, or C₃₋₆cyloalkyl provided that at least one is C₁₋₃ alkoxy; R⁴ is C₁₋₆ alkyl, C₁₋₃ alkyl-C₃₋₆cyloalkyl, C₃₋₆ heterocycloalkyl; or a pharmaceutically acceptable salt thereof.

In further embodiments, R¹, R², and R³ are each C₁₋₃ alkoxy; wherein R⁴ is C₄₋₆ alkyl, and wherein n and t are each 1, or a pharmaceutically acceptable salt thereof.

In further embodiments, R¹, R², and R³ are each methoxy; R⁴ is —CH₂-t-butyl, and n and t are each 1, or a pharmaceutically acceptable salt thereof.

In further embodiments, the compound is N-(4,6-dimethoxy-2-(4-methoxypiperidin-1-yl)pyrimidin-5-yl)-3,3-dimethylbutanamide, or a pharmaceutically acceptable salt thereof.

### Formula 151

In an embodiment, the Kv7 channel activator may be selected from one of the following compounds according to Formula 151. Such compounds are described in U.S. Pat. No. 9,353,048 issued May 31, 2016, and corresponding to U.S. application Ser. No. 14/353,842 filed Oct. 23, 2012; International Publication No. WO2013060097A1, published May 2, 2013, and corresponding to International Application No. PCT/CN2012/001423 filed Oct. 23, 2012; which are incorporated by reference in their entirety herein. In the case of any conflict of terminology in the context of Formula 152, these references incorporated by reference herein control.

In an embodiment, the Kv7 channel activator is a compound according to Formula 151:

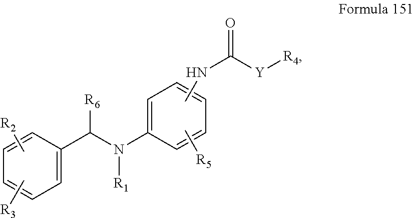

wherein, R₁ is a radical selected from the group consisting of C₂-C₈ alkenyl, C₅-C₇ cycloalkenyl and C₂-C₈ alkynyl; wherein, the C₂-C₈ alkenyl is unsubstituted or optionally substituted by hydroxyl, amino, halogen atom, phenyl or halogenated phenyl; the C₂-C₈ alkynyl is unsubstituted or optionally substituted by hydroxyl, amino, halogen atom, phenyl or halogenated phenyl; R₂ is a radical selected from the group consisting of F, Cl and methoxyl; R₃ is a radical selected from the group consisting of H, halogen atom and trifluoromethyl; Y is not present or Y is O; R₄ is a radical selected from the group consisting of C₁-C₆ alkyl, C₂-C₆ alkenyl, C₂-C₆ alkynyl and C₆-C₁₀ aryl; R₅ is a radical selected from the group consisting of H, halogen atom, amino and

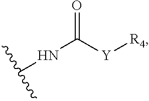

or R₅, together with adjacent

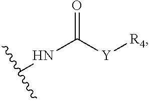

forms a fused-ring structure of

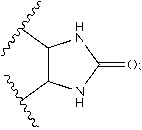 [SMILES: *C1NC(=O)NC1*]

R₆ is H or C₁-C₆ alkyl.

In further embodiments, the Kv7 Channel activator is selected from the group consisting of:

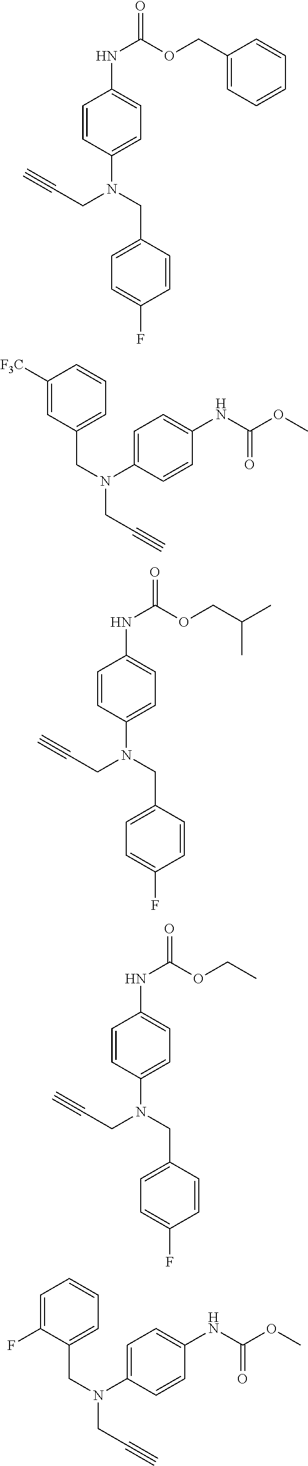

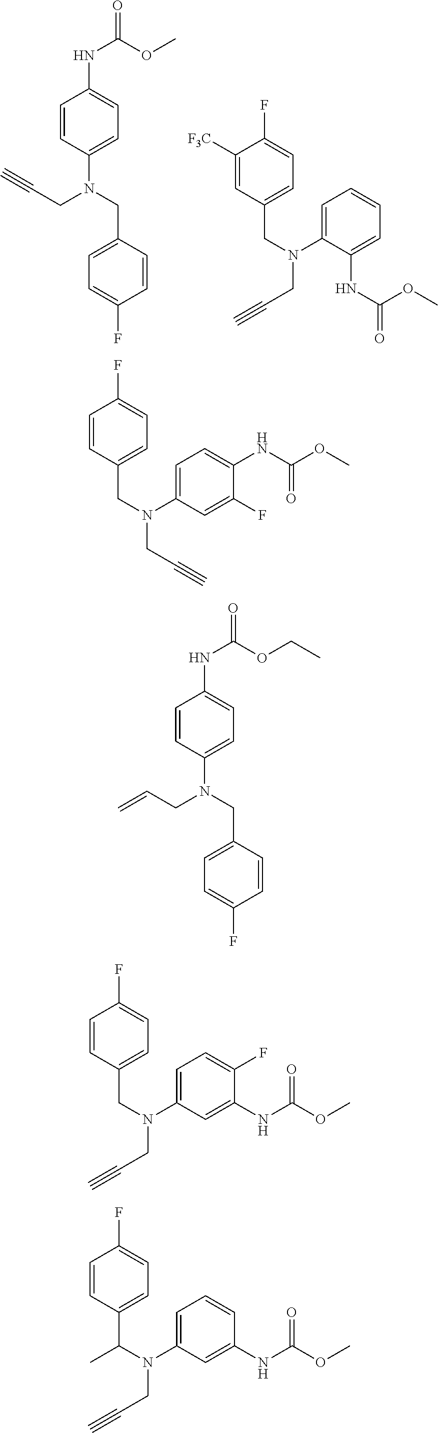

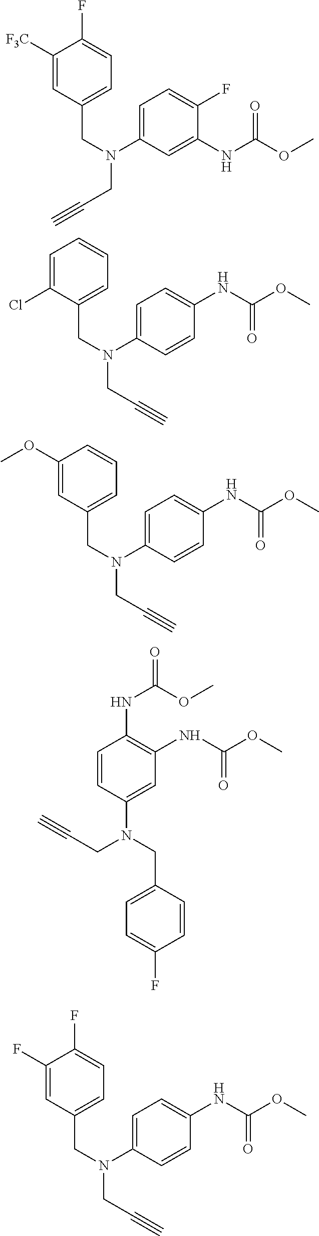

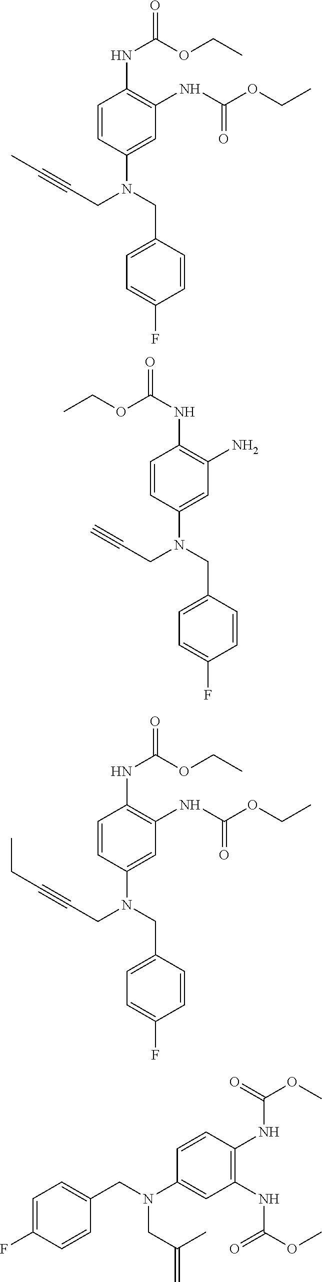

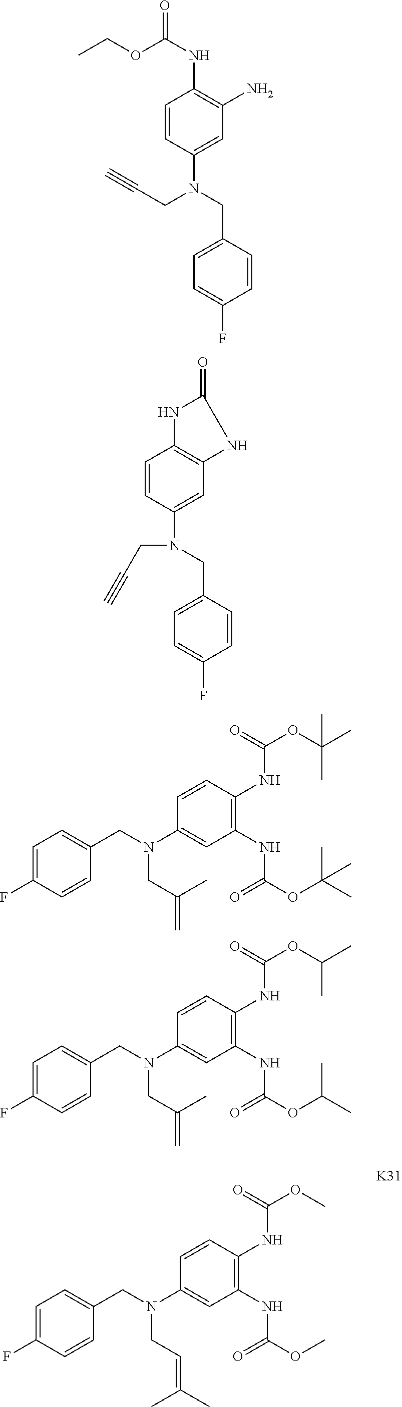

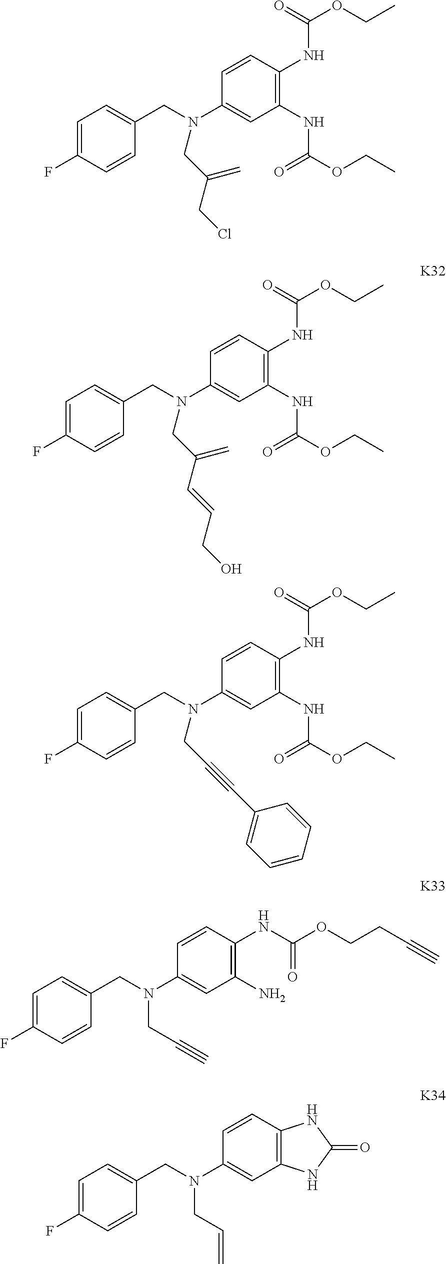 [SMILES: C=C(CCl)CN(Cc1ccc(F)cc1)c1ccc(NC(=O)OCC)c(NC(=O)OCC)c1 ; C=C(/C=C/CO)CN(Cc1ccc(F)cc1)c1ccc(NC(=O)OCC)c(NC(=O)OCC)c1 ; CCOC(=O)Nc1ccc(N(CC#Cc2ccccc2)Cc2ccc(F)cc2)cc1NC(=O)OCC ; C#CCCOC(=O)Nc1ccc(N(CC#C)Cc2ccc(F)cc2)cc1N ; C=CCN(Cc1ccc(F)cc1)c1ccc2[nH]c(=O)[nH]c2c1]

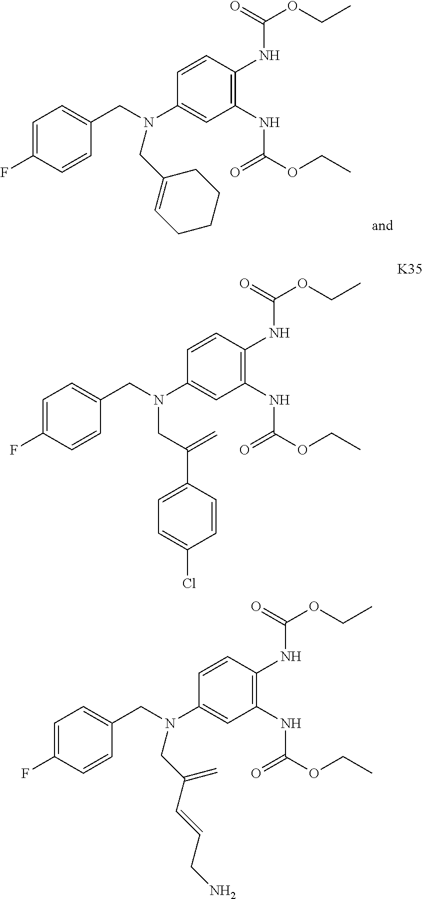 [SMILES: CCOC(=O)Nc1ccc(N(CC2=CCCCC2)Cc2ccc(F)cc2)cc1NC(=O)OCC ; C=C(CN(Cc1ccc(F)cc1)c1ccc(NC(=O)OCC)c(NC(=O)OCC)c1)c1ccc(Cl)cc1 ; C=C(/C=C/CN)CN(Cc1ccc(F)cc1)c1ccc(NC(=O)OCC)c(NC(=O)OCC)c1]

### Formula 152

In an embodiment, the Kv7 channel activator may be selected from one of the following compounds according to Formula 152. In another embodiment, the Kv7 channel activator may be selected from one of the following compounds. Such compounds are described in International Publication No. US20100057224A1, published Jun. 3, 2011, and corresponding to International Application No. PCT/US2010/057224 filed Nov. 18, 2010; U.S. Pat. No. 8,629,143 published Jan. 14, 2014, and corresponding to U.S. application Ser. No. 12/949,435 filed Nov. 18, 2010; which are incorporated by reference in their entirety herein. In the case of any conflict of terminology in the context of Formula 152, these references incorporated by reference herein control.

In an embodiment, the Kv7 channel activator is a compound according to Formula 152:

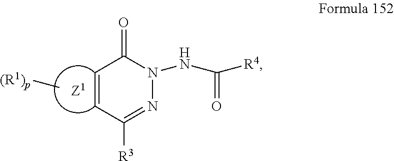

or a pharmaceutically acceptable salt, solvate, prodrug, or salt of a prodrug or solvate thereof, wherein Z is a ring fused with the pyridazine ring, selected from the group consisting of benzo, cycloalkyl, cycloalkenyl, heterocycle, and heteroaryl; R¹ is an optional substituent wherein each occurrence of R¹ is independently G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, —(CR^{za}R^{zb})ₘ—N(R^{a})SO₂NR^{a}R^{b}, or —(CR^{za}R^{zb})ₘ-G^{a}; p is 0, 1, 2, 3, or 4; R³ is hydrogen, alkyl, alkenyl, alkynyl, halogen, haloalkyl, —(CR^{3a}R^{3b})ₘ-G^{3a}, or G^{3a}; G^{3a}, at each occurrence, is independently aryl, cycloalkyl, or cycloalkenyl; each of which is optionally substituted; R⁴ is alkenyl, alkynyl, haloalkyl, G^{4a}, —(CR^{4a}R^{4b})ₙ-G^{4a}, or alkyl which is optionally substituted with one or two groups independently selected from the group consisting of S(R^{1a}), 0(R^{1a}) and N(R^{1a})₂; each occurrence of R^{1a} is independently hydrogen, G^{a}, —(CR^{za}R^{zb})ₘ-G^{a}, alkyl, haloalkyl, hydroxyalkyl, or alkoxyalkyl; G^{4a}, at each occurrence, is independently aryl, heteroaryl, heterocycle, cycloalkyl, or cycloalkenyl; each of which is optionally substituted; G^{3a} and G^{4a}, at each occurrence, are each independently unsubstituted or substituted with 1, 2, 3, or 4 substituents independently selected from the group consisting of G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ-G^{a}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; G^{a}, at each occurrence, is independently aryl, heteroaryl, heterocycle, cycloalkyl, or cycloalkenyl; each of which is independently unsubstituted or substituted with 1, 2, or 3 substituents selected from the group consisting of alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, (CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; R^{za}, R^{zb}, R^{3a}, R^{3b}, R^{4a}, and R^{4b}, at each occurrence, are each independently hydrogen, alkyl, halogen, or haloalkyl; R^{a} and R^{b}, at each occurrence, are each independently hydrogen, alkyl, or haloalkyl; and m and n, at each occurrence, are each independently 1, 2, 3, or 4; with the proviso that when Z¹ is benzo, p is 0 or 4, R¹ is halogen, R³ is G^{3a}, and G^{3a} is aryl, substituted with 1 or 2 substituents selected from the group consisting of alkyl and unsubstituted aryl, then R⁴ is other than unsubstituted aryl, unsubstituted alkyl, or haloalkyl.

In further embodiments, Z¹ is benzo, heteroaryl, or cycloalkyl.

In further embodiments, R³ is alkyl, halogen, haloalkyl, G^{3a}, or —(CR^{3a}R^{3b})ₘ-G^{3a}

In further embodiments, R⁴ is unsubstituted alkyl, haloalkyl, —C(R^{4a}R^{4b})ₙ-G^{4a}, or alkyl substituted with a —S(R^{1a}) group.

In further embodiments, Z¹ is benzo, cycloalkyl, or heteroaryl; and R⁴ is unsubstituted alkyl, haloalkyl, —C(R^{4a}R^{4b})ₙ-G^{4a}, or alkyl substituted with a —S(R^{1a}) group.

In further embodiments, Z¹ is benzo, cycloalkyl, or heteroaryl; and R⁴ is —C(R^{4a}R^{4b})ₙ-G^{4a}.

In further embodiments, Z¹ is benzo, cycloalkyl, or heteroaryl; R⁴ is —C(R^{4a}R^{4b})ₙ-G^{4a}. and R³ is alkyl, halogen, haloalkyl, G^{3a}, or —(CR^{3a}R^{3b})ₘ-G^{3a}

In further embodiments, Z¹ is benzo, cycloalkyl, or heteroaryl; R⁴ is —C(R^{4a}R^{4b})ₙ-G^{4a}; R³ is alkyl, halogen, haloalkyl, G^{3a}, or —(CR^{3a}R^{3b})ₘ-G^{3a}, and G^{3a} is aryl or cycloalkyl.

In further embodiments, Z¹ is benzo, cycloalkyl, or heteroaryl; and R⁴ is unsubstituted alkyl or haloalkyl.

In further embodiments, Z¹ is benzo, cycloalkyl, or heteroaryl; R⁴ is unsubstituted alkyl or haloalkyl; and R³ is alkyl, halogen, haloalkyl, G^{3a}, or —(CR^{3a}R^{3b})ₘ-G^{3a}

In further embodiments, Z¹ is benzo, cycloalkyl, or heteroaryl; R⁴ is unsubstituted alkyl or haloalkyl; R³ is alkyl, halogen, haloalkyl, G^{3a}, or —(CR^{3a}R^{3b})ₘ-G^{3a}, and G^{3a} is aryl or cycloalkyl.

In further embodiments, Z1 is benzo, cycloalkyl, or heteroaryl; and R⁴ is alkyl substituted with a —S(R^{1a}) group.

In further embodiments, Z1 is benzo, cycloalkyl, or heteroaryl; R⁴ is alkyl substituted with a —S(R^{1a}) group; and R³ is alkyl, halogen, haloalkyl, G^{3a}, or —(CR^{3a}R^{3b})ₘ-G^{3a}

In further embodiments, Z1 is benzo, cycloalkyl, or heteroaryl; R⁴ is alkyl substituted with a —S(R^{1a}) group; R³ is alkyl, halogen, haloalkyl, G^{3a}, or —(CR^{3a}R^{3b})ₘ-G^{3a}, and G^{3a} is aryl or cycloalkyl.

In further embodiments, the Kv7 Channel activator is selected from the group consisting of: 2-(4-chlorophenyl)-N-(4-isopropyl-1-oxophthalazin-2 (1H)-yl) acetamide; 2-(1-adamantyl)-N-[4-(4-bromophenyl)-1-oxophthalazin-2 (1H)-yl]acetamide; N-[4-(4-bromophenyl)-1-oxophthalazin-2 (1H)-yl]-2-(4-chlorophenyl) acetamide; 2-(4-chlorophenyl)-N-(1-oxo-4-phenylphthalazin-2 (1H)-yl) acetamide; 2-(3,5-difluorophenyl)-N-(4-isopropyl-1-oxophthalazin-2 (1H)-yl) acetamide; 2-(1-adamantyl)-N-(4-isopropyl-1-oxophthalazin-2 (1H)-yl) acetamide; 2-(1-adamantyl)-N-(8-oxo-5-phenylpyrido[2,3-d]pyridazin-7 (8H)-yl) acetamide; 2-(1-adamantyl)-N-(4-isopropyl-1-oxo-5,6,7,8-tetrahydro-5,8-ethanophthalazin-2 (1H)-yl) acetamide; 2-(1-adamantyl)-N-(4-oxo-7-phenylthieno[2,3-d]pyridazin-5 (4H)-yl) acetamide; 2-(3,5-difluorophenyl)-N-(4-oxo-7-phenylthieno[2,3-d]pyridazin-5 (4H)-yl) acetamide; 2-(3,5-difluorophenyl)-N-[1-oxo-4-(trifluoromethyl)phthalazin-2 (1H)-yl]acetamide; 2-(1-adamantyl)-N-[1-oxo-4-(trifluoromethyl)phthalazin-2 (1H)-yl]acetamide; 2-(4-chlorophenyl)-N-[1-oxo-4-(trifluoromethyl)phthalazin-2 (1H)-yl]acetamide; N-(5,8-difluoro-1-oxo-4-phenylphthalazin-2 (1H)-yl)-2-(4-fluorophenyl) acetamide; 2-(1-adamantyl)-N-(5,8-difluoro-1-oxo-4-phenylphthalazin-2 (1H)-yl) acetamide; 2-(4-chlorophenyl)-N-(5,8-difluoro-1-oxo-4-phenylphthalazin-2 (1H)-yl) acetamide; 2-(1-adamantyl)-N-(1-oxo-4-phenylphthalazin-2 (1H)-yl) acetamide; 2-(3,5-difluorophenyl)-N-(1-oxo-4-phenylphthalazin-2 (1H)-yl) acetamide; N-(4-chloro-1-oxophthalazin-2 (1H)-yl)-2-(3,5-difluorophenyl) acetamide; 2-(1-adamantyl)-N-(4-chloro-1-oxophthalazin-2 (1H)-yl) acetamide; N-(4-chloro-1-oxophthalazin-2 (1H)-yl)-2-(4-chlorophenyl) acetamide; 2-(4-chlorophenyl)-N-(4-cyclopropyl-1-oxophthalazin-2 (1H)-yl) acetamide; N-(4-cyclopropyl-1-oxophthalazin-2 (1H)-yl)-2-(3,5-difluorophenyl) acetamide; 2-(1-adamantyl)-N-(4-cyclopropyl-1-oxophthalazin-2 (1H)-yl) acetamide; 2-(2,3-difluorophenyl)-N-(1-oxo-4-phenylphthalazin-2 (1H)-yl) acetamide; 2-(4-fluorophenyl)-N-(1-oxo-4-phenylphthalazin-2 (1H)-yl) acetamide; 2-(2,5-difluorophenyl)-N-(1-oxo-4-phenylphthalazin-2 (1H)-yl) acetamide; 2-(4-chlorophenyl)-N-(4-methyl-1-oxophthalazin-2 (1H)-yl) acetamide; 2-(1-adamantyl)-N-(7-oxo-4-phenylthieno[2,3-d]pyridazin-6 (7H)-yl) acetamide; 2-[(1 S,2S,5S)-6,6-dimethylbicyclo[3.1.1]hept-2-yl]-N-(7-oxo-4-phenylthieno[2,3-d]pyridazin-6 (7H)-yl) acetamide; 2-(4-chlorophenyl)-N-(7-oxo-4-phenylthieno[2,3-d]pyridazin-6 (7H)-yl) acetamide; 2-(4-fluorophenyl)-N-(7-oxo-4-phenylthieno[2,3-d]pyridazin-6 (7H)-yl) acetamide; 2-(3,5-difluorophenyl)-N-(7-oxo-4-phenylthieno[2,3-d]pyridazin-6 (7H)-yl) acetamide; 2-[3,5-dimethyl-1-adamantyl]-N-(4-isopropyl-1-oxophthalazin-2 (1H)-yl) acetamide; 2-(3,5-difluorophenyl)-N-(6-fluoro-1-oxo-4-phenylphthalazin-2 (1H)-yl) acetamide; 2-(4-chlorophenyl)-N-(6-fluoro-1-oxo-4-phenylphthalazin-2 (1H)-yl) acetamide; 2-(1-adamantyl)-N-(6-fluoro-1-oxo-4-phenylphthalazin-2 (1H)-yl) acetamide; 2-(3,5-difluorophenyl)-N-(4-methyl-1-oxophthalazin-2 (1H)-yl) acetamide; 2-[1-(4-chlorophenyl)cyclopropyl]-N-[1-oxo-4-(trifluoromethyl)phthalazin-2 (1H)-yl]acetamide; 2-[1-(4-chlorophenyl)cyclobutyl]-N-[1-oxo-4-(trifluoromethyl)phthalazin-2 (1H)-yl]acetamide; 2-(2-naphthyl)-N-[1-oxo-4-(trifluoromethyl)phthalazin-2(1H)-yl]acetamide; 3-(4-chlorophenyl)-3-methyl-N-[1-oxo-4-(trifluoromethyl)phthalazin-2 (1H)-yl]butanamide; 2-cyclopentyl-N-[1-oxo-4-(trifluoromethyl)phthalazin-2 (1H)-yl]acetamide; 2.2-difluoro-N-[1-oxo-4-(trifluoromethyl)phthalazin-2 (1H)-yl]-2-phenylacetamide; 2-cyclobutyl-N-[1-oxo-4-(trifluoromethyl)phthalazin-2 (1H)-yl]acetamide; N-[1-oxo-4-(trifluoromethyl)phthalazin-2 (1H)-yl]-2-[4-(trifluoromethyl)phenyl]acetamide; 2-[4-(dimethylamino)phenyl]-N-[1-oxo-4-(trifluoromethyl)phthalazin-2 (1H)-yl]acetamide; 3.3-dimethyl-N-[1-oxo-4-(trifluoromethyl)phthalazin-2 (1H)-yl]butanamide; 2-[4-(methylsulfonyl)phenyl]-N-[1-oxo-4-(trifluoromethyl)phthalazin-2 (1H)-yl]acetamide; N-[1-oxo-4-(trifluoromethyl)phthalazin-2 (1H)-yl]-3-phenylpropanamide; N-[1-oxo-4-(trifluoromethyl)phthalazin-2 (1H)-yl]-2-(1-phenylcyclopropyl) acetamide; 3-methyl-N-[1-oxo-4-(trifluoromethyl)phthalazin-2 (1H)-yl]-3-phenylbutanamide; N-[1-oxo-4-(trifluoromethyl)phthalazin-2 (1H)-yl]-2-(3-thienyl) acetamide; N-[1-oxo-4-(trifluoromethyl)phthalazin-2 (1H)-yl]-2-(2-thienyl) acetamide; 2-(5-chloro-2-thienyl)-N-[1-oxo-4-(trifluoromethyl)phthalazin-2 (1H)-yl]acetamide; 2-(5-methyl-2-thienyl)-N-[1-oxo-4-(trifluoromethyl)phthalazin-2 (1H)-yl]acetamide; N-[1-oxo-4-(trifluoromethyl)phthalazin-2 (1H)-yl]-2-phenylacetamide; (±)-2-(e o-bicyclo[2.2.1]heptan-2-yl)-N-[1-oxo-4-(trifluoromethyl)phthalazin-2 (1H)-yl]acetamide; 2-(4-chloro-3-fluorophenyl)-N-[1-oxo-4-(trifluoromethyl)phthalazin-2 (1H)-yl]acetamide; 2-(3-fluoroadamantan-1-yl)-N-(4-isopropyl-1-oxophthalazin-2 (1H)-yl) acetamide; 2-(3-hydroxyadamantan-1-yl)-N-(4-isopropyl-1-oxophthalazin-2 (1H)-yl) acetamide; N-(4-tert-butyl-1-oxophthalazin-2 (1H)-yl)-2-cyclopentylacetamide; (±)-2-(e o-bicyclo[2.2.1]heptan-2-yl)-N-(4-tert-butyl-1-oxophthalazin-2 (1H)-yl) acetamide; N-(4-tert-butyl-1-oxophthalazin-2 (1H)-yl)-3-methyl-3-phenylbutanamide; N-(4-tert-butyl-1-oxophthalazin-2 (1H)-yl)-2-(4-chlorophenyl) acetamide; 2-[(1 S,2S,4R)-bicyclo[2.2.1]hept-2-yl]-N-(4-cyclobutyl-1-oxophthalazin-2 (1H)-yl) acetamide; 2-[(1 S,2S,4S)-bicyclo[2.2.1]hept-5-en-2-yl]-N-(4-cyclobutyl-1-oxophthalazin-2(1H)-yl) acetamide; (±)-2-(e o-bicyclo[2.2.1]heptan-2-yl)-N-(4-cyclobutyl-1-oxophthalazin-2 (1H)-yl) acetamide; N-(4-cyclobutyl-1-oxophthalazin-2 (1H)-yl)-2-(3,5-difluorophenyl) acetamide; 2-(4-chlorophenyl)-N-(4-cyclobutyl-1-oxophthalazin-2 (1H)-yl) acetamide; (±)-2-(e o-bicyclo[2.2.1]heptan-2-yl)-N-(4-cyclopentyl-1-oxophthalazin-2 (1H)-yl) acetamide; 2-(4-chlorophenyl)-N-(4-cyclopentyl-1-oxophthalazin-2 (1H)-yl) acetamide; N-(4-cyclopentyl-1-oxophthalazin-2 (1H)-yl)-2-(3,5-difluorophenyl) acetamide; (±)-2-(e o-bicyclo[2.2.1]heptan-2-yl)-N-(4-cyclohexyl-1-oxophthalazin-2 (1H)-yl) acetamide; 2-(adamantan-1-yl)-N-(4-cyclohexyl-1-oxophthalazin-2 (1H)-yl) acetamide; 2-(4-chlorophenyl)-N-(4-cyclohexyl-1-oxophthalazin-2 (1H)-yl) acetamide; N-(4-cyclohexyl-1-oxophthalazin-2 (1H)-yl)-2-(3,5-difluorophenyl) acetamide; (±)-4-(3-{[(ero-bicyclo[2.2.1]heptan-2-yl) acetyl]amino}-4-oxo-3,4-dihydrophthalazin-1-yl)benzoic acid; (±)-methyl 4-(3-{[ero-bicyclo[2.2.1]hept-2-ylacetyl]amino}-4-oxo-3,4-dihydrophthalazin-1-yl)benzoate; methyl 4-(3-{[(4-chlorophenyl) acetyl]amino}-4-oxo-3,4-dihydrophthalazin-1-yl)benzoate; (±)-4-(3-{[e o-bicyclo[2.2.1]hept-2-ylacetyl]amino}-4-oxo-3,4-dihydrophthalazin-1-yl)-N,N-dimethylbenzamide; 3-methyl-N-(1-oxo-4-phenylphthalazin-2(1H)-yl)-3-phenylbutanamide; 2-(2,4-dichlorophenyl)-N-(1-oxo-4-phenylphthalazin-2 (1H)-yl) acetamide; (±)-2-(e o-bicyclo[2.2.1]heptan-2-yl)-N-[4-(4-bromophenyl)-1-oxophthalazin-2 (1H)-yl]acetamide; N-[4-(4-bromophenyl)-1-oxophthalazin-2 (1H)-yl]-3-methyl-3-phenylbutanamide; N-[4-(4-chlorophenyl)-1-oxophthalazin-2 (1H)-yl]-2-(1-methylcyclopentyl) acetamide; N-[4-(4-chlorophenyl)-1-oxophthalazin-2 (1H)-yl]-2-[1-(trifluoromethyl)cyclopentyl]acetamide; N-[4-(4-chlorophenyl)-1-oxophthalazin-2 (1H)-yl]-2-[(1 S,2S,5R)-3,3-difluoro-6,6-dimethylbicyclo[3.1.1]hept-2-yl]acetamide; N-[4-(4-chlorophenyl)-1-oxophthalazin-2 (1H)-yl]-3-methyl-3-phenylbutanamide; N-[4-(4-chlorophenyl)-1-oxophthalazin-2 (1H)-yl]-2-fluoro-2-phenylacetamide; N-[4-(4-chlorophenyl)-1-oxophthalazin-2 (1H)-yl]-2-phenylacetamide; N-[4-(4-chlorophenyl)-1-oxophthalazin-2 (1H)-yl]-2-(morpholin-4-yl) acetamide; N-[4-(4-chlorophenyl)-1-oxophthalazin-2 (1H)-yl]-2-(pyridin-3-yl) acetamide; N-[4-(4-chlorophenyl)-1-oxophthalazin-2 (1H)-yl]-2-(pyridin-2-yl) acetamide; N-[4-(4-chlorophenyl)-1-oxophthalazin-2 (1H)-yl]-2-(3,4-dichlorophenyl) acetamide; N-[4-(4-chlorophenyl)-1-oxophthalazin-2 (1H)-yl]-2-(3,5-dimethoxyphenyl) acetamide; N-[4-(4-chlorophenyl)-1-oxophthalazin-2 (1H)-yl]-2-(3,5-dimethylphenyl) acetamide; N-[4-(4-chlorophenyl)-1-oxophthalazin-2 (1H)-yl]-2-[3-(trifluoromethoxy)phenyl]acetamide; N-[4-(4-chlorophenyl)-1-oxophthalazin-2 (1H)-yl]-2-[4-(trifluoromethyl)phenyl]acetamide; N-[4-(4-chlorophenyl)-1-oxophthalazin-2 (1H)-yl]-2-[3-(trifluoromethyl)phenyl]acetamide; N-[4-(4-chlorophenyl)-1-oxophthalazin-2 (1H)-yl]-2-[4-(dimethylamino)phenyl]acetamide; 2-(4-bromophenyl)-N-[4-(4-chlorophenyl)-1-oxophthalazin-2 (1H)-yl]acetamide; 2-(3-chlorophenyl)-N-[4-(4-chlorophenyl)-1-oxophthalazin-2 (1H)-yl]acetamide; N-[4-(4-chlorophenyl)-1-oxophthalazin-2 (1H)-yl]-2-(4-methoxyphenyl) acetamide; N-[4-(4-chlorophenyl)-1-oxophthalazin-2 (1H)-yl]-2-(3-methoxyphenyl) acetamide; N-[4-(4-chlorophenyl)-1-oxophthalazin-2 (1H)-yl]-2-(4-hydroxyphenyl) acetamide; N-[4-(4-chlorophenyl)-1-oxophthalazin-2 (1H)-yl]-2-(4-methylphenyl) acetamide; N-[4-(4-chlorophenyl)-1-oxophthalazin-2 (1H)-yl]-2-(3-methylphenyl) acetamide; N-[4-(4-chlorophenyl)-1-oxophthalazin-2 (1H)-yl]-2-cyclopentylacetamide; N-[4-(4-chlorophenyl)-1-oxophthalazin-2 (1H)-yl]-4-methylpentanamide; N-[4-(4-chlorophenyl)-1-oxophthalazin-2 (1H)-yl]-2-[4-(methylsulfonyl)phenyl]acetamide; N-[4-(4-chlorophenyl)-1-oxophthalazin-2 (1H)-yl]-2-(5-chloro-2-thienyl) acetamide; (±)-2-(e o-bicyclo[2.2.1]hept-2-yl)-N-[4-(4-chlorophenyl)-1-oxophthalazin-2(1H)-yl]acetamide; 2-(4-chloro-3-fluorophenyl)-N-[4-(4-chlorophenyl)-1-oxophthalazin-2 (1H)-yl]acetamide; N-[4-(4-chlorophenyl)-1-oxophthalazin-2 (1H)-yl]-2-[(1 S,2S,5S)-6,6-dimethylbicyclo[3.1.1]hept-2-yl]acetamide; 2-(adamantan-1-yl)-N-[4-(4-chlorophenyl)-1-oxophthalazin-2 (1H)-yl]acetamide; 2-(4-chlorophenyl)-N-[4-(4-chlorophenyl)-1-oxophthalazin-2 (1H)-yl]acetamide; N-[4-(4-chlorophenyl)-1-oxophthalazin-2(1H)-yl]-2-(3,5-difluorophenyl) acetamide; N-[4-(4-chlorophenyl)-1-oxophthalazin-2 (1H)-yl]-2-(1-phenylcyclopentyl) acetamide; N-[4-(4-chlorophenyl)-1-oxophthalazin-2 (1H)-yl]-2-phenylcyclopropanecarboxamide; N-[4-(4-chlorophenyl)-1-oxophthalazin-2 (1H)-yl]-2-(2-naphthyl) acetamide; N-[4-(4-chlorophenyl)-1-oxophthalazin-2(1H)-yl]-2-(1-naphthyl) acetamide; N-[4-(4-chlorophenyl)-1-oxophthalazin-2 (1H)-yl]-4,4,4-trifluorobutanamide; N-[4-(4-chlorophenyl)-1-oxophthalazin-2 (1H)-yl]-3,3,3-trifluoropropanamide; (±)-2-(e o-bicyclo[2.2.1]hept-2-yl)-N-[4-(3-chlorophenyl)-1-oxophthalazin-2 (1H)-yl]acetamide; 2-(adamantan-1-yl)-N-[4-(3-chlorophenyl)-1-oxophthalazin-2 (1H)-yl]acetamide; 2-(4-chlorophenyl)-N-[4-(3-chlorophenyl)-1-oxophthalazin-2 (1H)-yl]acetamide; N-[4-(3-chlorophenyl)-1-oxophthalazin-2 (1H)-yl]-2-(3,5-difluorophenyl) acetamide; 2-[(1 S,2S,4R)-bicyclo[2.2.1]hept-2-yl]-N-[4-(4-fluorophenyl)-1-oxophthalazin-2 (1H)-yl]acetamide; 2-[(1 S,2S,4S)-bicyclo[2.2.1]hept-5-en-2-yl]-N-[4-(4-fluorophenyl)-1-oxophthalazin-2 (1H)-yl]acetamide; (±)-2-(e o-bicyclo[2.2.1]hept-2-yl)-N-[4-(4-fluorophenyl)-1-oxophthalazin-2(1H)-yl]acetamide; 2-(4-chlorophenyl)-N-[4-(4-fluorophenyl)-1-oxophthalazin-2 (1H)-yl]acetamide; 2-(3,5-difluorophenyl)-N-[4-(4-fluorophenyl)-1-oxophthalazin-2 (1H)-yl]acetamide; (±)-2-(e o-bicyclo[2.2.1]hept-2-yl)-N-[4-(2,4-difluorophenyl)-1-oxophthalazin-2 (1H)-yl]acetamide; 2-(4-chlorophenyl)-N-[4-(4-methylphenyl)-1-oxophthalazin-2 (1H)-yl]acetamide; 2-(3,5-difluorophenyl)-N-[4-(4-methylphenyl)-1-oxophthalazin-2 (1H)-yl]acetamide; 2-[(1 S,2S,5S)-6,6-dimethylbicyclo[3.1.1]hept-2-yl]-N-[4-(4-methylphenyl)-1-oxophthalazin-2 (1H)-yl]acetamide; 2-(adamantan-1-yl)-N-[4-(4-methylphenyl)-1-oxophthalazin-2 (1H)-yl]acetamide; N-(4-benzyl-1-oxophthalazin-2 (1H)-yl)-2-[1-(trifluoromethyl)cyclopentyl]acetamide; N-(4-benzyl-1-oxophthalazin-2 (1H)-yl)-3-methyl-3-phenylbutanamide; (±)-N-(4-benzyl-1-oxophthalazin-2 (1H)-yl)-2-(e o-bicyclo[2.2.1]hept-2-yl) acetamide; N-(4-benzyl-1-oxophthalazin-2 (1H)-yl)-2-(4-chlorophenyl) acetamide; N-(4-benzyl-1-oxophthalazin-2 (1H)-yl)-2-(3,5-difluorophenyl) acetamide; (±)-2-(e o-bicyclo[2.2.1]hept-2-yl)-N-[4-(4-chlorobenzyl)-1-oxophthalazin-2 (1H)-yl]acetamide; N-[4-(4-chlorobenzyl)-1-oxophthalazin-2 (1H)-yl]-2-(4-chlorophenyl) acetamide; N-[4-(4-chlorobenzyl)-1-oxophthalazin-2 (1H)-yl]-2-(3,5-difluorophenyl) acetamide; 2-[(1 S,2S,4R)-bicyclo[2.2.1]hept-2-yl]-N-{1-oxo-4-[4-(trifluoromethyl)phenyl]phthalazin-2 (1H)-yl}acetamide; 3-methyl-N-{1-oxo-4-[4-(trifluoromethyl)phenyl]phthalazin-2 (1H)-yl}-3-phenylbutanamide; 2-(adamantan-1-yl)-N-{1-oxo-4-[4-(trifluoromethyl)phenyl]phthalazin-2 (1H)-yl}acetamide; (±)-2-(exo-bicyclo[2.2.1]hept-2-yl)-N-{1-oxo-4-[4-(trifluoromethyl)phenyl]phthalazin-2 (1H)-yl}acetamide; (±)-2-(e o-bicyclo[2.2.1]hept-2-yl)-N-[4-(4-methoxyphenyl)-1-oxophthalazin-2(1H)-yl]acetamide; N-[4-(4-methoxyphenyl)-1-oxophthalazin-2 (1H)-yl]-2-(4-methylcyclohexyl) acetamide; 2-(3,5-difluorophenyl)-N-[4-(4-methoxyphenyl)-1-oxophthalazin-2 (1H)-yl]acetamide; 2-(adamantan-1-yl)-N-[4-(4-methoxyphenyl)-1-oxophthalazin-2 (1H)-yl]acetamide; (±)-2-(e o-bicyclo[2.2.1]hept-2-yl)-N-[4-(2,5-dimethylphenyl)-1-oxophthalazin-2 (1H)-yl]acetamide; 2-(adamantan-1-yl)-N-[4-(2,5-dimethylphenyl)-1-oxophthalazin-2 (1H)-yl]acetamide; 2-(4-chlorophenyl)-N-[4-(2,5-dimethylphenyl)-1-oxophthalazin-2 (1H)-yl]acetamide; 2-(3,5-difluorophenyl)-N-[4-(2,5-dimethylphenyl)-1-oxophthalazin-2 (1H)-yl]acetamide; 2-[(1 S,2S,4S)-bicyclo[2.2.1]hept-5-en-2-yl]-N-[4-(2,4-dimethylphenyl)-1-oxophthalazin-2(1H)-yl]acetamide; (±)-2-(e o-bicyclo[2.2.1]hept-2-yl)-N-[4-(2,4-dimethylphenyl)-1-oxophthalazin-2 (1H)-yl]acetamide; 2-(4-chlorophenyl)-N-[4-(2,4-dimethylphenyl)-1-oxophthalazin-2(1H)-yl]acetamide; 2-(3,5-difluorophenyl)-N-[4-(2,4-dimethylphenyl)-1-oxophthalazin-2 (1H)-yl]acetamide; (±)-2-(e o-bicyclo[2.2.1]hept-2-yl)-N-[4-(3,4-dimethylphenyl)-1-oxophthalazin-2 (1H)-yl]acetamide; 2-(adamantan-1-yl)-N-[4-(3,4-dimethylphenyl)-1-oxophthalazin-2(1H)-yl]acetamide; 2-(4-chlorophenyl)-N-[4-(3,4-dimethylphenyl)-1-oxophthalazin-2 (1H)-yl]acetamide; 2-(3,5-difluorophenyl)-N-[4-(3,4-dimethylphenyl)-1-oxophthalazin-2(1H)-yl]acetamide; (±)-2-(e o-bicyclo[2.2.1]hept-2-yl)-N-[1-oxo-4-(2-phenylethyl)phthalazin-2(1H)-yl]acetamide; (±)-2-(e o-bicyclo[2.2.1]hept-2-yl)-N-[4-(4-isopropylphenyl)-1-oxophthalazin-2(1H)-yl]acetamide; 2-(adamantan-1-yl)-N-[4-(4-isopropylphenyl)-1-oxophthalazin-2(1H)-yl]acetamide; 2-(4-chlorophenyl)-N-[4-(4-isopropylphenyl)-1-oxophthalazin-2 (1H)-yl]acetamide; 2-(3,5-difluorophenyl)-N-[4-(4-isopropylphenyl)-1-oxophthalazin-2 (1H)-yl]acetamide; (±)-2-(ero-bicyclo[2.2.1]hept-2-yl)-N-[1-oxo-4-(1-phenylcyclopropyl)phthalazin-2 (1H)-yl]acetamide; 2-(adamantan-1-yl)-N-[4-isopropyl-1-oxo-7-(trifluoromethyl)phthalazin-2 (1H)-yl]acetamide; 2-(adamantan-1-yl)-N-[7-bromo-4-(4-methoxyphenyl)-1-oxophthalazin-2 (1H)-yl]acetamide; 2-(adamantan-1-yl)-N-[6-bromo-4-(4-methoxyphenyl)-1-oxophthalazin-2 (1H)-yl]acetamide; N-[6-bromo-4-(4-methoxyphenyl)-1-oxophthalazin-2 (1H)-yl]-2-(3,5-difluorophenyl) acetamide; N-[7-bromo-4-(4-methoxyphenyl)-1-oxophthalazin-2 (1H)-yl]-2-(3,5-difluorophenyl) acetamide; 2-(3-bromoadamantan-1-yl)-N-(7-oxo-4-phenylthieno[2,3-d]pyridazin-6 (7H)-yl) acetamide; 2-(3-fluoroadamantan-1-yl)-N-(7-oxo-4-phenylthieno[2,3-d]pyridazin-6 (7H)-yl) acetamide; 2-(3-hydroxyadamantan-1-yl)-N-(7-oxo-4-phenylthieno[2,3-d]pyridazin-6 (7H)-yl) acetamide; N-[4-(4-chlorophenyl)-5,8-difluoro-1-oxophthalazin-2 (1H)-yl]-2-(3,5-difluorophenyl) acetamide; 2-(4-chlorophenyl)-N-[4-(4-chlorophenyl)-5,8-difluoro-1-oxophthalazin-2 (1H)-yl]acetamide; 2-(adamantan-1-yl)-N-[4-(4-chlorophenyl)-5,8-difluoro-1-oxophthalazin-2 (1H)-yl]acetamide; (±)-2-(e o-bicyclo[2.2.1]hept-2-yl)-N-[4-(4-chlorophenyl)-5,8-difluoro-1-oxophthalazin-2 (1H)-yl]acetamide; 2-(adamantan-1-yl)-N-(1-oxo-4-phenyl-5,6,7,8-tetrahydrophthalazin-2 (1H)-yl) acetamide; 2-(3,5-difluorophenyl)-N-(1-oxo-4-phenyl-5,6,7,8-tetrahydrophthalazin-2 (1H)-yl) acetamide; 2-(4-chlorophenyl)-N-(1-oxo-4-phenyl-5,6,7,8-tetrahydrophthalazin-2 (1H)-yl) acetamide; 2-(3,5-difluorophenyl)-N-(1-oxo-4-phenyl-1,5,6,7-tetrahydro-2H-cyclopenta[d]pyridazin-2-yl) acetamide; 2-(adamantan-1-yl)-N-(1-oxo-4-phenyl-1,5,6,7-tetrahydro-2H-cyclopenta[d]pyridazin-2-yl) acetamide; 2-(4-chlorophenyl)-N-(1-oxo-4-phenyl-1,5,6,7-tetrahydro-2H-cyclopenta[d]pyridazin-2-yl) acetamide; 2-(methylthio)-N-[1-oxo-4-(trifluoromethyl)phthalazin-2 (1H)-yl]acetamide; 2-(adamantan-1-ylthio)-N-[1-oxo-4-(trifluoromethyl)phthalazin-2 (1H)-yl]acetamide; 2-(adamantan-1-ylthio)-N-(1-oxo-4-phenylphthalazin-2 (1H)-yl) acetamide; and 2-(1,3-benzodioxol-5-yl)-N-[4-(4-chlorophenyl)-1-oxophthalazin-2 (1H)-yl]acetamide.

### Formula 153

In an embodiment, the Kv7 channel activator may be selected from one of the following compounds according to Formula 153. Such compounds are described in U.S. Pat. No. 8,629,143, issued Jan. 14, 2014, and corresponding to US Application No. U.S. Pat. No. 8,629,143B2 filed Nov. 18, 2010; International Publication No. WO2011066168A1, published Jun. 3, 2011, and corresponding to International Application No. PCT/US2010/057224 filed Nov. 18, 2010; which are incorporated by reference in their entirety herein. In the case of any conflict of terminology in the context of Formula 153, these references incorporated by reference herein control.

In an embodiment, the Kv7 channel activator is a compound according to Formula 153:

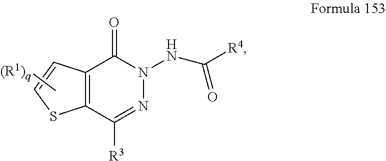

or a pharmaceutically acceptable salt, solvate, prodrug, or salt of a prodrug or solvate thereof, wherein, R¹ is an optional substituent wherein each occurrence of R¹ is independently G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, —(CR^{za}R^{zb})ₘ—N(R^{a})SO₂NR^{a}R^{b}, or —(CR^{za}R^{zb})ₘ-G^{a}; p is 0, 1, 2, 3, or 4; R³ is hydrogen, alkyl, alkenyl, alkynyl, halogen, haloalkyl, —(CR^{3a}R^{3b})ₘ-G^{3a}, or G^{3a}, G^{3a}, at each occurrence, is independently aryl, cycloalkyl, or cycloalkenyl; each of which is optionally substituted; R⁴ is alkenyl, alkynyl, haloalkyl, G^{4a}, —(CR^{4a}R^{4b})ₙ-G^{4a}, or alkyl which is optionally substituted with one or two groups independently selected from the group consisting of S(R^{1a}), 0(R^{1a}) and N(R^{1a})₂; each occurrence of R^{1a} is independently hydrogen, G^{a}, —(CR^{za}R^{zb})ₘ-G^{a}, alkyl, haloalkyl, hydroxyalkyl, or alkoxyalkyl; G^{4a}, at each occurrence, is independently aryl, heteroaryl, heterocycle, cycloalkyl, or cycloalkenyl; each of which is optionally substituted; G^{3a} and G^{4a}, at each occurrence, are each independently unsubstituted or substituted with 1, 2, 3, or 4 substituents independently selected from the group consisting of G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ-G^{a}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb}) n N(R^{a})CONR^{a}R^{b}, and —(CR^{za}R^{zb})ₘ—N(R^{a})SO₂NR^{a}R^{b}; G^{a}, at each occurrence, is independently aryl, heteroaryl, heterocycle, cycloalkyl, or cycloalkenyl; each of which is independently unsubstituted or substituted with 1, 2, or 3 substituents selected from the group consisting of alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}, R^{za}, R^{zb}, R^{3a}, R^{3b}, R^{4a}, and R^{4b}, at each occurrence, are each independently hydrogen, alkyl, halogen, or haloalkyl; R^{a} and R^{b}, at each occurrence, are each independently hydrogen, alkyl, or haloalkyl; and m and n, at each occurrence, are each independently 1, 2, 3, or 4.

### Formula 154

In an embodiment, the Kv7 channel activator may be selected from one of the following compounds according to Formula 154. Such compounds are described in U.S. Pat. No. 8,629,143, issued Jan. 14, 2014, and corresponding to US Application No. U.S. Pat. No. 8,629,143B2 filed Nov. 18, 2010; International Publication No. WO2011066168A1, published Jun. 3, 2011, and corresponding to International Application No. PCT/US2010/057224 filed Nov. 18, 2010; which are incorporated by reference in their entirety herein. In the case of any conflict of terminology in the context of Formula 154, these references incorporated by reference herein control.

In an embodiment, the Kv7 channel activator is a compound according to Formula 154:

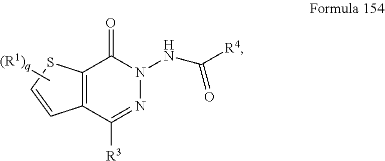

or a pharmaceutically acceptable salt, solvate, prodrug, or salt of a prodrug or solvate thereof, wherein R¹ is an optional substituent wherein each occurrence of R¹ is independently G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, —(CR^{za}R^{zb})ₘ—N(R^{a})SO₂NR^{a}R^{b}, or —(CR^{za}R^{zb})ₘ-G^{a}; p is 0, 1, 2, 3, or 4; R³ is hydrogen, alkyl, alkenyl, alkynyl, halogen, haloalkyl, —(CR^{3a}R^{3b})ₘ-G^{3a}, or G^{3a}, G^{3a}, at each occurrence, is independently aryl, cycloalkyl, or cycloalkenyl; each of which is optionally substituted; R⁴ is alkenyl, alkynyl, haloalkyl, G^{4a}, —(CR^{4a}R^{4b})ₙ-G^{4a}, or alkyl which is optionally substituted with one or two groups independently selected from the group consisting of S(R^{1a}), 0(R^{1a}) and N(R^{1a})₂; each occurrence of R^{1a} is independently hydrogen, G^{a}, —(CR^{za}R^{zb})ₘ-G^{a}, alkyl, haloalkyl, hydroxyalkyl, or alkoxyalkyl; G^{4a}, at each occurrence, is independently aryl, heteroaryl, heterocycle, cycloalkyl, or cycloalkenyl; each of which is optionally substituted; G^{3a} and G^{4a}, at each occurrence, are each independently unsubstituted or substituted with 1, 2, 3, or 4 substituents independently selected from the group consisting of G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a}) SO₂NR^{a}R^{b}, —(CR^{za}R^{zb})ₘ-G^{a}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; G^{a}, at each occurrence, is independently aryl, heteroaryl, heterocycle, cycloalkyl, or cycloalkenyl; each of which is independently unsubstituted or substituted with 1, 2, or 3 substituents selected from the group consisting of alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; R^{za}, R^{zb}, R^{3a}, R^{3b}, R^{4a}, and R^{4b}, at each occurrence, are each independently hydrogen, alkyl, halogen, or haloalkyl; R^{a} and R^{b}, at each occurrence, are each independently hydrogen, alkyl, or haloalkyl; and m and n, at each occurrence, are each independently 1, 2, 3, or 4; and q is 0, 1, or 2.

### Formula 155

In an embodiment, the Kv7 channel activator may be selected from one of the following compounds according to Formula 155. Such compounds are described in U.S. Pat. No. 8,629,143, issued Jan. 14, 2014, and corresponding to US Application No. U.S. Pat. No. 8,629,143B2 filed Nov. 18, 2010; International Publication No. WO2011066168A1, published Jun. 3, 2011, and corresponding to International Application No. PCT/US2010/057224 filed Nov. 18, 2010; which are incorporated by reference in their entirety herein. In the case of any conflict of terminology in the context of Formula 155, these references incorporated by reference herein control.

In an embodiment, the Kv7 channel activator is a compound according to Formula 155:

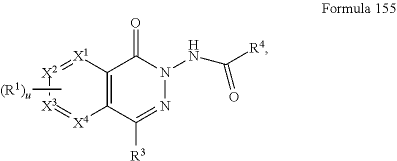

or a pharmaceutically acceptable salt, solvate, prodrug, or salt of a prodrug or solvate thereof, wherein R¹ is an optional substituent wherein each occurrence of R¹ is independently G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, —(CR^{za}R^{zb})ₘ—N(R^{a})SO₂NR^{a}R^{b}, or —(CR^{za}R^{zb})ₘ-G^{a}; p is 0, 1, 2, 3, or 4; R³ is hydrogen, alkyl, alkenyl, alkynyl, halogen, haloalkyl, —(CR^{3a}R^{3b})ₘ-G^{3a}, or G^{3a}, G^{3a}, at each occurrence, is independently aryl, cycloalkyl, or cycloalkenyl; each of which is optionally substituted; R⁴ is alkenyl, alkynyl, haloalkyl, G^{4a}, —(CR^{4a}R^{4b})ₙ-G^{4a}, or alkyl which is optionally substituted with one or two groups independently selected from the group consisting of S(R^{1a}), 0(R^{1a}) and N(R^{1a})₂; each occurrence of R^{1a} is independently hydrogen, G^{a}, —(CR^{za}R^{zb})ₘ-G^{a}, alkyl, haloalkyl, hydroxyalkyl, or alkoxyalkyl; G^{4a}, at each occurrence, is independently aryl, heteroaryl, heterocycle, cycloalkyl, or cycloalkenyl; each of which is optionally substituted; G^{3a} and G^{4a}, at each occurrence, are each independently unsubstituted or substituted with 1, 2, 3, or 4 substituents independently selected from the group consisting of G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ-G^{a}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; G^{a}, at each occurrence, is independently aryl, heteroaryl, heterocycle, cycloalkyl, or cycloalkenyl; each of which is independently unsubstituted or substituted with 1, 2, or 3 substituents selected from the group consisting of alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; R^{za}, R^{zb}, R^{3a}, R^{3b}, R^{4a}, and R^{4b}, at each occurrence, are each independently hydrogen, alkyl, halogen, or haloalkyl; R^{a} and R^{b}, at each occurrence, are each independently hydrogen, alkyl, or haloalkyl; and m and n, at each occurrence, are each independently 1, 2, 3, or 4; and u is 0, 1, 2, or 3.

### Formula 156

In an embodiment, the Kv7 channel activator may be selected from one of the following compounds according to Formula 156. Such compounds are described in U.S. Pat. No. 8,629,143, issued Jan. 14, 2014, and corresponding to US Application No. U.S. Pat. No. 8,629,143B2 filed Nov. 18, 2010; International Publication No. WO2011066168A1, published Jun. 3, 2011, and corresponding to International Application No. PCT/US2010/057224 filed Nov. 18, 2010; which are incorporated by reference in their entirety herein. In the case of any conflict of terminology in the context of Formula 156, these references incorporated by reference herein control.

In an embodiment, the Kv7 channel activator is a compound according to Formula 156:

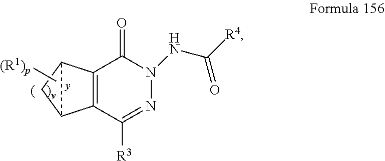

or a pharmaceutically acceptable salt, solvate, prodrug, or salt of a prodrug or solvate thereof, wherein R¹ is an optional substituent wherein each occurrence of R¹ is independently G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, —(CR^{za}R^{zb})ₘ—N(R^{a})SO₂NR^{a}R^{b}, or —(CR^{za}R^{zb})ₘ-G^{a}; p is 0, 1, 2, 3, or 4; R³ is hydrogen, alkyl, alkenyl, alkynyl, halogen, haloalkyl, —(CR^{3a}R^{3b})ₘ-G^{3a}, or G^{3a}, G^{3a}, at each occurrence, is independently aryl, cycloalkyl, or cycloalkenyl; each of which is optionally substituted; R⁴ is alkenyl, alkynyl, haloalkyl, G^{4a}, (CR^{4a}R^{4b})ₙ-G^{4a}, or alkyl which is optionally substituted with one or two groups independently selected from the group consisting of S(R^{1a}), 0(R^{1a}) and N(R^{1a})₂; each occurrence of R^{1a} is independently hydrogen, G^{a}, —(CR^{za}R^{zb})ₘ-G^{a}, alkyl, haloalkyl, hydroxyalkyl, or alkoxyalkyl; G^{4a}, at each occurrence, is independently aryl, heteroaryl, heterocycle, cycloalkyl, or cycloalkenyl; each of which is optionally substituted; G^{3a} and G^{4a}, at each occurrence, are each independently unsubstituted or substituted with 1, 2, 3, or 4 substituents independently selected from the group consisting of G^{a}, alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ-G^{a}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; G^{a}, at each occurrence, is independently aryl, heteroaryl, heterocycle, cycloalkyl, or cycloalkenyl; each of which is independently unsubstituted or substituted with 1, 2, or 3 substituents selected from the group consisting of alkyl, alkenyl, alkynyl, halogen, haloalkyl, oxo, —CN, —NO₂, —OR^{a}, —OC(O)R^{a}, —OC(O)NR^{a}R^{b}, —NR^{a}R^{b}, —SR^{a}, —S(O)R^{a}, —S(O)₂R^{a}, —S(O)₂NR^{a}R^{b}, —C(O)R^{a}, —C(O)OR^{a}, —C(O)NR^{a}R^{b}, —N(R^{a})COOR^{b}, —N(R^{a})CONR^{a}R^{b}, —N(R^{a})SO₂NR^{a}R^{b}, —(CR^{za}R^{zb})ₘ—CN, —(CR^{za}R^{zb})ₘ—NO₂, —(CR^{za}R^{zb})ₘ—OR^{a}, —(CR^{za}R^{zb})ₘ—OC(O)R^{a}, —(CR^{za}R^{zb})ₘ—OC(O)NR^{a}R^{b}, —(CR^{za}R^{zb})ₘ—SR^{a}, —(CR^{za}R^{zb})ₘ—S(O)R^{a}, —(CR^{za}R^{zb})ₘ—S(O)₂R^{a}, —(CR^{za}R^{zb})ₘ—S(O)₂NR^{a}R^{b}, —(CR^{za}R^{zb})ₘ—C(O)R^{a}, —(CR^{za}R^{zb})ₘ—C(O)OR^{a}, —(CR^{za}R^{zb})ₘ—C(O)NR^{a}R^{b}, —(CR^{za}R^{zb})ₘ—NR^{a}R^{b}, —(CR^{za}R^{zb})ₘ—N(R^{a})COOR^{b}, —(CR^{za}R^{zb})ₘ—N(R^{a})CONR^{a}R^{b}, and —(CR^{za}R^{zb})ₘ—N(R^{a})SO₂NR^{a}R^{b}; R^{za}, R^{zb}, R^{3a}, R^{3b}, R^{4a}, and R^{4b}, at each occurrence, are each independently hydrogen, alkyl, halogen, or haloalkyl; R^{a} and R^{b}, at each occurrence, are each independently hydrogen, alkyl, or haloalkyl; and m and n, at each occurrence, are each independently 1, 2, 3, or 4; v is 1, 2, or 3; and
y is absent, a bond, —CH₂—, or —CH₂CH₂-.

### Formula 157

In an embodiment, the Kv7 channel activator may be selected from one of the following compounds according to Formula 157. Such compounds are described in US Publication No. US20110257146A1, published Oct. 20, 2011, and corresponding to U.S. application Ser. No. 12/223,136 filed Jan. 25, 2007; International Publication No. WO2007087424A2, published Aug. 2, 2007, and corresponding to International Application No. PCT/US2007/002116 filed Jan. 26, 2007; which are incorporated by reference in their entirety herein. In the case of any conflict of terminology in the context of Formula 157, these references incorporated by reference herein control.

In an embodiment, the Kv7 channel activator is a compound according to Formula 157:

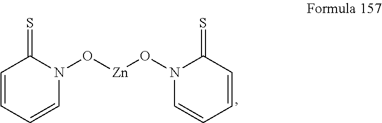

In further embodiments, the Kv7 Channel activator is selected from the group consisting of: zinc-5,7-Diiodo-8-hydroxyquinoline and zinc-8-Hydroxyquinoline.

In further embodiments, the Kv7 Channel activator is selected from the group consisting of: zinc-pyrrolidine dithiocarbamate, zinc-diethyldithiocarbamate, zinc-disulfiram and zinc-dimethyldithiocarbamate.

In further embodiments, the Kv7 Channel activator is selected from the group consisting of: zinc-vitamin E and zinc-vitamin A.

### Formula 158

In an embodiment, the Kv7 channel activator may be selected from one of the following compounds according to Formula 158. Such compounds are described in US Publication No. US20110257146A1, published Oct. 20, 2011, and corresponding to U.S. application Ser. No. 12/223,136 filed Jan. 25, 2007; International Publication No. WO2007087424A2, published Aug. 2, 2007, and corresponding to International Application No. PCT/US2007/002116 filed Jan. 26, 2007; which are incorporated by reference in their entirety herein. In the case of any conflict of terminology in the context of Formula 158, these references incorporated by reference herein control.

In an embodiment, the Kv7 channel activator is a compound according to Formula 158:

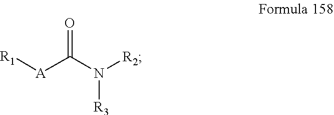

wherein, A is a bond, CH₂, CHR_{b}, CH₂S, CHRIS, CH₂O, CH₂NR_{c}, or NH; Rb is alkyl; R_{c} is H or S(O)ₘ-aryl; R₁ is an optionally substituted alkyl, an optionally substituted aryl, an optionally substituted cycloalkyl, or an optionally substituted heteroaryl; R₂ is an optionally substituted alkyl, an optionally substituted alkenyl, an optionally substituted aryl, an optionally substituted cycloalkyl, an optionally substituted heteroaryl, an optionally substituted heterocyclic, an optionally substituted aralkyl,

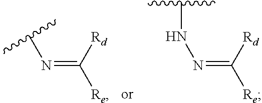

R₃ is H or alkyl; each R_{d} and Rₑ is independently an optionally substituted alkyl, an optionally substituted aryl, or R_{d} and Rₑ together form an optionally substituted cycloalkyl; and m is 0, 1, or 2.

### Formula 159

In an embodiment, the Kv7 channel activator may be selected from one of the following compounds according to Formula 159. Such compounds are described in US Publication No. US20110257146A1, published Oct. 20, 2011, and corresponding to U.S. application Ser. No. 12/223,136 filed Jan. 25, 2007; International Publication No. WO2007087424A2, published Aug. 2, 2007, and corresponding to International Application No. PCT/US2007/002116 filed Jan. 26, 2007; which are incorporated by reference in their entirety herein. In the case of any conflict of terminology in the context of Formula 159, these references incorporated by reference herein control.

In an embodiment, the Kv7 channel activator is a compound according to Formula 159:

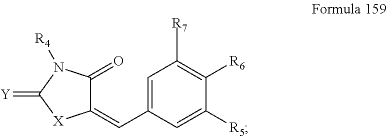

wherein, R₄ is H, an optionally substituted alkyl, an optionally substituted alkenyl, alkynyl, allyl, or an optionally substituted aryl; R₅ is H, hal, or hydroxyl; R₆ is H, hal, hydroxyl, NH2, a mono- or di-substituted amine, or an optionally substituted alkoxy; R₇ is H, hal, hydroxyl, an optionally substituted alkoxy, or nitro; X is S or NRₐ; Y is O, S, or NRₐ; and each Rₐ is independently H or an optionally substituted aryl.

In further embodiments, the Kv7 Channel activator is selected from the group consisting of: N-Benzo[g]quinolin-4-yl-N′-(2-diethylamino-ethyl)-benzene-1,4-diamine; 2-[2-(3,4-Dihydroxy-phenyl)-2-oxo-ethylsulfanyl]-4,6-dimethyl-nicotinonitrile; 2-[2-(4-Methoxy-phenyl)-2-oxo-ethylsulfanyl]-4-(5-methyl-furan-2-yl)-5,6,7,8-tetrahydro-quinoline-3-carbonitrile; 6-Methyl-4-(5-methyl-furan-2-yl)-2-(2-oxo-2-phenyl-ethylsulfanyl)-nicotinonitrile; 2-(2-Oxo-2-thiophen-2-yl-ethylsulfanyl)-4-pyridin-4-yl-5,6,7,8-tetrahydro-quinoline-3-carbonitrile; 2-(3,5-Diiodo-2-methoxy-phenyl)-2,3,5,6,7,8-hexahydro-1H-benzo[4,5]thieno[2,3-d]pyrimidin-4-one; 2,2,2-Trifluoro-1-[1-(2,2,2-trifluoro-1-hydroxy-1-trifluoromethyl-ethyl)-1H-pyrrol-2-yl]-ethanone; 1,5-Diphenyl-1H-pyrazole-3-carboxylic acid tert-butylamide; 3-(4-Bromo-phenyl)-5-(3-phenyl-allylidene)-dihydro-pyrimidine-2,4-dione; 2-Amino-7-hydroxy-6-[(2-iodo-phenylimino)-methyl]-4-phenyl-4H-chromene-3-carbonitrile; 3-(1H-Benzoimidazol-2-yl)-6-nitro-chromen-2-ylideneamine; 6-Methoxy-3-(4-nitro-phenyl)-chroman-2-one; 2-(Benzo[1,2,5]thiadiazol-4-yliminomethyl)-benzo[b]thiophen-3-ol; 4-[3-(4-Bromo-phenyl)-3-oxo-propenylamino]-N-(4,6-dimethyl-pyrimidin-2-yl)-benzenesulfonamide; 2-[(5-Nitro-furan-2-ylmethylene)-amino]-benzamide; 2-Benzo[4,5]imidazo[1,2-c]quinazolin-6-yl-phenylamine; Dimethyl-phenyl)-5-(3-phenyl-allylidene)-pyrimidine-2,4,6-trione; 4-(4-Cyclohexyl-phenyl)-thiazol-2-ylamine; or 4-(4-Cyclohexyl-phenyl)-thiazol-2-ylamine.

### Formula 160

In an embodiment, the Kv7 channel activator may be selected from one of the following compounds according to Formula 160. Such compounds are described in International Publication No. WO2004060880A1, published Jul. 22, 2004, and corresponding to International Application No. PCT/US2003/039352 filed Dec. 11, 2003; U.S. Pat. No. 6,933,308, issued Aug. 23, 2005, and corresponding to U.S. application Ser. No. 10/730,781 filed Dec. 9, 2003; which are incorporated by reference in their entirety herein. In the case of any conflict of terminology in the context of Formula 160, these references incorporated by reference herein control.

In an embodiment, the Kv7 channel activator is a compound according to Formula 160;

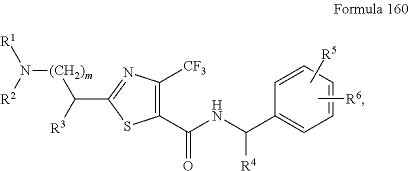

wherein, R¹ is C₁₋₆alkyl, C₃₋₇cycloalkyl, —(CH₂)₁₋₄C₃₋₇cycloalkyl, —(CH₂)₂₋₄N(C₁₋₆alkyl)₂, —(CH₂)₂₋₄OC₁₋₆alkyl,

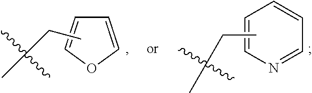

R² is hydrogen, C₁₋₆alkyl, or —(CH₂)₂₋₄OC₁₋₆alkyl; or where R¹ and R² taken together are —CH₂CH₂XCH₂CH₂—, where X is a chemical bond, CH₂, CHOH, NH, NCH₃, NCOCH₃, O, or S; R³ is hydrogen or hydroxy, provided that where R³ is hydroxy, m is not 0; R⁴ is hydrogen, C₁₋₆alkyl, hydroxymethyl, or trifluoromethyl; R⁵ is halogen, C₁₋₆alkyl, C₁₋₂perfluoroalkyl, C₁₋₆alkoxy, C₁₋₂perfluoroalkoxy, —N(R⁴)₂, N-morpholinyl, or pyridyl; R⁶ is hydrogen, halogen, or C₁₋₆alkoxy; m is 0 or 1.

In further embodiments, R³ is hydrogen and m is 1.

In further embodiments, R⁴ is methyl.

In further embodiments, the Kv7 Channel activator is selected from the group consisting of: 2-[2-(4-morpholinyl)ethyl]-N-[1-[3-(3-pyridinyl)phenyl]ethyl]-4-(trifluoromethyl)-5-thiazolecarboxamide; N-[1-[3-(dimethylamino)phenyl]ethyl]-2-[2-(1-pyrrolidinyl)ethyl]-4-(trifluoromethyl)-5-thiazolecarboxamide; 2-[2-(1-pyrrolidinyl)ethyl]-4-(trifluoromethyl)-N-[1-[3-(trifluoromethyl)phenyl]ethyl]-5-thiazolecarboxamide; 2-[2-[(2-furanylmethyl)methylamino]ethyl]-N-[(1S)-1-[3-(4-morpholinyl)phenyl]ethyl]-4-(trifluoromethyl)-5-thiazolecarboxamide; 2-[2-[(2-furanylmethyl)methylamino]ethyl]-N-[1-[3-(3-pyridinyl)phenyl]ethyl]-4-(trifluoromethyl)-5-thiazolecarboxamide; 2-[2-(1-pyrrolidinyl)ethyl]-N-[1-[3-(trifluoromethoxy)phenyl]ethyl]-4-(trifluoromethyl)-5-thiazolecarboxamide; 2-[2-[(2-furanylmethyl)methylamino]ethyl]-N-[1-[3-(trifluoromethoxy)phenyl]ethyl]-4-(trifluoromethyl)-5-thiazolecarboxamide; 2-[2-(diethylamino)ethyl]-N-[1-[3-(trifluoromethoxy)phenyl]ethyl]-4-(trifluoromethyl)-5-thiazolecarboxamide; 2-[2-(diethylamino)ethyl]-N-[1-[3-(dimethylamino)phenyl]ethyl]-4-(trifluoromethyl)-5-thiazolecarboxamide; 2-[2-[ethyl(4-pyridinylmethyl)amino]ethyl]-4-(trifluoromethyl)-N-[1-[3-(trifluoromethyl)phenyl]ethyl]-5-thiazolecarboxamide; 2-[2-(4-thiomorpholinyl)ethyl]-4-(trifluoromethyl)-N-[1-[3-(trifluoromethyl)phenyl]ethyl]-5-thiazolecarboxamide; 2-[[2-[2-(dimethylamino)ethyl]methylamino]ethyl]-N-[1-[3-(trifluoromethoxy)phenyl]ethyl]-4-(trifluoromethyl)-5-thiazolecarboxamide; 2-[2-[[2-(dimethylamino)ethyl]methylamino]ethyl]-4-(trifluoromethyl)-N-[1-[3-(trifluoromethyl)phenyl]ethyl]-5-thiazolecarboxamide; 2-[2-(4-methyl-1-piperazinyl)ethyl]-N-[1-[3-(trifluoromethoxy)phenyl]ethyl]-4-(trifluoromethyl)-5-thiazolecarboxamide; 2-[2-(1-piperidinyl)ethyl]-N-[1-[3-(trifluoromethoxy)phenyl]ethyl]-4-(trifluoromethyl)-5-thiazolecarboxamide; N-[1-[3-(dimethylamino)phenyl]ethyl]-2-[2-(1-piperidinyl)ethyl]-4-(trifluoromethyl)-5-thiazolecarboxamide; 2-[2-(1-piperidinyl)ethyl]-4-(trifluoromethyl)-N-[1-[3-(trifluoromethyl)phenyl]ethyl]-5-thiazolecarboxamide; N-[(1S)-1-[3-(4-morpholinyl)phenyl]ethyl]-2-[2-(1-piperidinyl)ethyl]-4-(trifluoromethyl)-5-thiazolecarboxamide; 2-[2-(4-hydroxy-1-piperidinyl)ethyl]-N-[1-[3-(trifluoromethoxy)phenyl]ethyl]-4-(trifluoromethyl)-5-thiazolecarboxamide; 2-[2-(4-hydroxy-1-piperidinyl)ethyl]-4-(trifluoromethyl)-N-[1-[3-(trifluoromethyl)phenyl]ethyl]-5-thiazolecarboxamide; 2-[2-[(cyclopropylmethyl)propylamino]ethyl]-N-[1-[3-(trifluoromethoxy)phenyl]ethyl]-4-(trifluoromethyl)-5-thiazolecarboxamide; 2-[2-[(cyclopropylmethyl)propylamino]ethyl]-N-[1-[3-(dimethylamino)phenyl]ethyl]-4-(trifluoromethyl)-5-thiazolecarboxamide; 2-[2-[(cyclopropylmethyl)propylamino]ethyl]-4-(trifluoromethyl)-N-[1-[3-(trifluoromethyl)phenyl]ethyl]-5-thiazolecarboxamide; 2-[2-(diethylamino)ethyl]-N-[(1S)-1-[3-(3-pyridinyl)phenyl]ethyl]-4-(trifluoromethyl)-5-thiazolecarboxamide; 2-[2-(1-piperidinyl)ethyl]-N-[(1S)-1-[3-(3-pyridinyl)phenyl]ethyl]-4-(trifluoromethyl)-5-thiazolecarboxamide; 2-[2-[(1-ethylpropyl)amino]ethyl]-N-[1-[3-(trifluoromethoxy)phenyl]ethyl]-4-(trifluoromethyl)-5-thiazolecarboxamide; 2-[2-[(1-ethylpropyl)amino]ethyl]-4-(trifluoromethyl)-N-[1-[3-(trifluoromethyl)phenyl]ethyl]-5-thiazolecarboxamide; 2-[2-[(2-furanylmethyl)amino]ethyl]-N-[(1S)-1-[3-(3-pyridinyl)phenyl]ethyl]-4-(trifluoromethyl)-5-thiazolecarboxamide; 2-[2-(cyclopentylamino)ethyl]-N-[1-[3-(trifluoromethoxy)phenyl]ethyl]-4-(trifluoromethyl)-5-thiazolecarboxamide; 2-[2-(cyclopentylamino)ethyl]-N-[(1S)-1-[3-(3-pyridinyl)phenyl]ethyl]-4-(trifluoromethyl)-5-thiazolecarboxamide; 2-[2-[bis(2-methoxyethyl)amino]ethyl]-N-[1-[3-(trifluoromethoxy)phenyl]ethyl]-4-(trifluoromethyl)-5-thiazolecarboxamide; 2-[2-[bis(2-methoxyethyl)amino]ethyl]-4-(trifluoromethyl)-N-[1-[3-(trifluoromethyl)phenyl]ethyl]-5-thiazolecarboxamide; 2-[2-[bis(2-methoxyethyl)amino]ethyl]-N-[(1S)-1-[3-(3-pyridinyl)phenyl]ethyl]-4-(trifluoromethyl)-5-thiazolecarboxamide; 2-[2-(4-morpholinyl)ethyl]-N-[(1S)-1-[3-(4-morpholinyl)phenyl]ethyl]-4-(trifluoromethyl)-5-thiazolecarboxamide; N-[1-[3-(dimethylamino)phenyl]ethyl]-2-[2-(4-thiomorpholinyl)ethyl]-4-(trifluoromethyl)-5-thiazolecarboxamide; 2-[2-[[2-(dimethylamino)ethyl]methylamino]ethyl]-N-[1-[3-(dimethylamino)phenyl]ethyl]-4-(trifluoromethyl)-5-thiazolecarboxamide; and 2-[2-(4-methyl-1-piperazinyl)ethyl]-N-[(1S)-1-[3-(4-morpholinyl)phenyl]ethyl]-4-(trifluoromethyl)-5-thiazolecarboxamide.

In further embodiments, R³ is hydroxy and m is 1.

In further embodiments, R⁴ is methyl.

In further embodiments, the Kv7 Channel activator is selected from the group consisting of: 2-[1-hydroxy-2-(1-piperidinyl)ethyl]-N-[1-[3-(trifluoromethoxy)phenyl]ethyl]-4-(trifluoromethyl)-5-thiazolecarboxamide; 2-[1-hydroxy-2-(1-pyrrolidinyl)ethyl]-4-(trifluoromethyl)-N-[1-[3-(trifluoromethyl)phenyl]ethyl]-5-thiazolecarboxamide; 2-[2-[(2-furanylmethyl)methylamino]-1-hydroxyethyl]-4-(trifluoromethyl)-N-[1-[3-(trifluoromethyl)phenyl]ethyl]-5-thiazolecarboxamide; 2-[2-[(cyclopropylmethyl)propylamino]-1-hydroxyethyl]-N-[1-[3-(trifluoromethoxy)phenyl]ethyl]-4-(trifluoromethyl)-5-thiazolecarboxamide; 2-[2-(diethylamino)-1-hydroxyethyl]-N-[(1S)-1-[3-(4-morpholinyl)phenyl]ethyl]-4-(trifluoromethyl)-5-thiazolecarboxamide; 2-[2-(diethylamino)-1-hydroxyethyl]-N-[(1S)-1-[3-(3-pyridinyl)phenyl]ethyl]-4-(trifluoromethyl)-5-thiazolecarboxamide; 2-[1-hydroxy-2-(4-morpholinyl)ethyl]-N-[1-[3-(trifluoromethoxy)phenyl]ethyl]-4-(trifluoromethyl)-5-thiazolecarboxamide; 2-[1-hydroxy-2-(4-morpholinyl)ethyl]-4-(trifluoromethyl)-N-[1-[3-(trifluoromethyl)phenyl]ethyl]-5-thiazolecarboxamide; 2-[1-hydroxy-2-(4-methyl-1-piperazinyl)ethyl]-4-(trifluoromethyl)-N-[1-[3-(trifluoromethyl)phenyl]ethyl]-5-thiazolecarboxamide; 2-[1-hydroxy-2-(4-methyl-1-piperazinyl)ethyl]-N-[(1S)-1-[3-(4-morpholinyl)phenyl]ethyl]-4-(trifluoromethyl)-5-thiazolecarboxamide; 2-[1-hydroxy-2-(4-methyl-1-piperazinyl)ethyl]-N-[(1S)-1-[3-(3-pyridinyl)phenyl]ethyl]-4-(trifluoromethyl)-5-thiazolecarboxamide; 2-[1-hydroxy-2-(1-piperidinyl)ethyl]-4-(trifluoromethyl)-N-[1-[3-(trifluoromethyl)phenyl]ethyl]-5-thiazolecarboxamide; 2-[1-hydroxy-2-(1-pyrrolidinyl)ethyl]-N-[1-[3-(trifluoromethoxy)phenyl]ethyl]-4-(trifluoromethyl)-5-thiazolecarboxamide; N-[1-[3-(dimethylamino)phenyl]ethyl]-2-[1-hydroxy-2-(1-pyrrolidinyl)ethyl]-4-(trifluoromethyl)-5-thiazolecarboxamide; 2-[1-hydroxy-2-(1-pyrrolidinyl)ethyl]-N-[(1S)-1-[3-(4-morpholinyl)phenyl]ethyl]-4-(trifluoromethyl)-5-thiazolecarboxamide; 2-[2-[(cyclopropylmethyl)propylamino]-1-hydroxyethyl]-4-(trifluoromethyl)-N-[1-[3-(trifluoromethyl)phenyl]ethyl]-5-thiazolecarboxamide; and 2-[2-(4-acetyl-1-piperazinyl)-1-hydroxyethyl]-4-(trifluoromethyl)-N-[1-[3-(trifluoromethyl)phenyl]ethyl]-5-thiazolecarboxamide.

In further embodiments, the compound has a stereochemical configuration according to the formula:

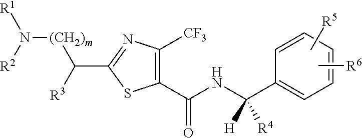

In further embodiments, the compound is according to the formula:

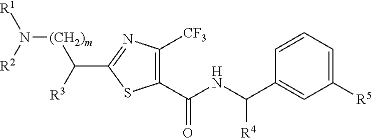

Wherein, R¹ is C₁₋₆alkyl, C₃₋₇cycloalkyl, —(CH₂)₁₋₄C₃₋₇cycloalkyl, —(CH₂)₂₋₄N(C₁₋₆alkyl)₂, —(CH₂)₂₋₄OC₁₋₆alkyl,

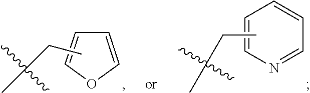

R² is hydrogen C₁₋₆alkyl, or —(CH₂)₂₋₄OC₁₋₆alkyl; or where R¹ and R² taken together are —CH₂CH₂XCH₂CH₂—, where X is a chemical bond, CH₂, CHOH, NH, NCH₃, NCOCH₃, O, or S; R³ is hydrogen or hydroxy, provided that where R³ is hydroxy, m is not 0; R⁴ is hydrogen, C₁₋₆alkyl, hydroxymethyl, or trifluoromethyl; R⁵ is halogen, C₁₋₆alkyl, C₁₋₂perfluoroalkyl, C₁₋₆alkoxy, C₁₋₂perfluoroalkoxy, —N(R⁴)₂, N-morpholinyl, or pyridyl; and m is 0 or 1; or a pharmaceutically acceptable salt thereof.

### Formula 161

In an embodiment, the Kv7 channel activator may be selected from one of the following compounds according to Formula 161. Such compounds are described in U.S. Pat. No. 7,144,881 issued Dec. 5, 2006, and corresponding to U.S. application Ser. No. 10/919,184 filed Nov. 21, 2003; International Publication No. WO2004047738A2, published Jun. 10, 2004, and corresponding to International Application No. PCT/US2003/037305 filed Nov. 21, 2003; which are incorporated by reference in their entirety herein. In the case of any conflict of terminology in the context of Formula 161, these references incorporated by reference herein control.

In an embodiment, the Kv7 channel activator is a compound according to Formula 161:

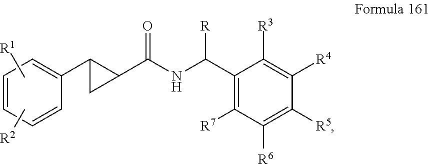

wherein, R is C₁₋₄ alkyl, CF₃ or hydroxymethyl; R¹ and R² are each independently hydrogen, C₁₋₄ alkyl, halogen or morpholin-4-yl; R⁴ is selected from the group consisting of optionally substituted morpholin-4-yl, pyridinyl, pyrimidinyl, piperazinyl, and pyrazinyl, in which said substituent is independently selected from the group consisting of C₁₋₄alkyl, dimethylamino, hydroxymethyl, chloro and fluoro; R⁵ is hydrogen or fluoro; or R⁴ and R⁵ taken together are —CH═CH—CH═CH— or —CH₂CH₂O—; and R³, R⁶ and R⁷ are each independently selected from hydrogen or fluoro.

### Formula 162

In an embodiment, the Kv7 channel activator may be selected from one of the following compounds according to Formula 162. Such compounds are described in U.S. Pat. No. 7,144,881 issued Dec. 5, 2006, and corresponding to U.S. application Ser. No. 10/919,184 filed Nov. 21, 2003; International Publication No. WO2004047738A2, published Jun. 10, 2004, and corresponding to International Application No. PCT/US2003/037305 filed Nov. 21, 2003; which are incorporated by reference in their entirety herein. In the case of any conflict of terminology in the context of Formula 162, these references incorporated by reference herein control.

In an embodiment, the Kv7 channel activator is a compound according to Formula 162:

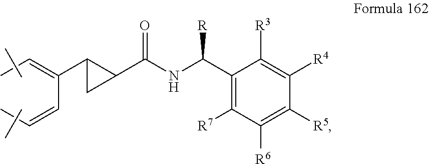

wherein, R is methyl or hydroxymethyl; R¹ and R² are each independently hydrogen, C₁₋₄ alkyl, halogen or morpholin-4-yl; R⁴ is selected from the group consisting of optionally substituted morpholin-4-yl, pyridinyl, pyrimidinyl, piperazinyl, and pyrazinyl, in which said substituent is independently selected from the group consisting of C₁₋₄alkyl, dimethylamino, hydroxymethyl, chloro and fluoro; R⁵ is hydrogen or fluoro; or R⁴ and R⁵ taken together are —CH═CH—CH═CH— or —CH₂CH₂O—; and R³, R⁶ and R⁷ are each independently selected from hydrogen or fluoro.

In further embodiments, the Kv7 Channel activator is selected from the group consisting of: 2-(2-fluoro-phenyl)-cyclopropanecarboxylic acid [1-(2,3-dihydro-benzofuran-5-yl)-ethyl]-amide; 2-(3-fluoro-phenyl)-cyclopropanecarboxylic acid [1-(2,3-dihydro-benzofuran-5-yl)-ethyl]-amide; 2-(4-fluoro-phenyl)-cyclopropanecarboxylic acid [1-(2,3-dihydro-benzofuran-5-yl)-ethyl]-amide; 2-(2-fluoro-phenyl)-cyclopropanecarboxylic acid (2-hydroxy-1-naphthalen-2-yl-ethyl)-amide; 2-(3-fluoro-phenyl)-cyclopropanecarboxylic acid (2-hydroxy-1-naphthalen-2-yl-ethyl)-amide; 2-(4-fluoro-phenyl)-cyclopropanecarboxylic acid (2-hydroxy-1-naphthalen-2-yl-ethyl)-amide; 2-(2,5-difluoro-phenyl)-cyclopropanecarboxylic acid (2-hydroxy-1-naphthalen-2-yl-ethyl)-amide; 2-(2-fluoro-phenyl)-cyclopropanecarboxylic acid [1-(4-fluoro-3-morpholin-4-yl-phenyl)-2-hydroxy-ethyl]-amide; 2-(3-fluoro-phenyl)-cyclopropanecarboxylic acid [1-(4-fluoro-3-morpholin-4-yl-phenyl)-2-hydroxy-ethyl]-amide; 2-(4-fluoro-phenyl)-cyclopropanecarboxylic acid [1-(4-fluoro-3-morpholin-4-yl-phenyl)-2-hydroxy-ethyl]-amide; 2-(2,5-difluoro-phenyl)-cyclopropanecarboxylic acid [1-(4-fluoro-3-morpholin-4-yl-phenyl)-2-hydroxy-ethyl]-amide; 2-(4-fluoro-phenyl)-cyclopropanecarboxylic acid (1-naphthalen-2-yl-ethyl)-amide; 2-(2,5-difluoro-phenyl)-cyclopropanecarboxylic acid (1-naphthalen-2-yl-ethyl)-amide; 2-(4-fluoro-phenyl)-cyclopropanecarboxylic acid {1-[3-(3-dimethylamino-pyrrolidin-1-yl)-phenyl]-ethyll}-amide; 2-(2,5-difluoro-phenyl)-cyclopropanecarboxylic acid {1-[3-(3-dimethylamino-pyrrolidin-1-yl)-phenyl]-ethyl}-amide; 2-(3-fluoro-phenyl)-cyclopropanecarboxylic acid [1-(3-pyridin-3-yl-phenyl)-ethyl]-amide; 2-(2,5-difluoro-phenyl)-cyclopropanecarboxylic acid [1-(3-pyridin-3-yl-phenyl)-ethyl]-amide; (S)-2-phenyl-cyclopropanecarboxylic acid [1-(3-pyridin-3-yl-phenyl)-ethyl]-amide; (S)-2-(3-fluoro-phenyl)-cyclopropanecarboxylic acid {1-[3-(6-fluoro-pyridin-3-yl)-phenyl]-ethyl}-amide; (S)-2-phenyl-cyclopropanecarboxylic acid {1-[3-(2-fluoro-pyridin-3-yl)-phenyl]-ethyl}-amide; and(S)-2-(2-fluoro-phenyl)-cyclopropanecarboxylic acid {1-[3-(2-fluoro-pyridin-3-yl)-phenyl]-ethyl}-amide; or a pharmaceutically acceptable salt thereof.

### Formula 163

In an embodiment, the Kv7 channel activator may be selected from one of the following compounds according to Formula 163. Such compounds are described in International Publication No. WO2004047743 A2, published Jun. 10, 2004, and corresponding to International Application No. PCT/US2003/037348 filed Nov. 21, 2003; U.S. Pat. No. 7,045,551 issued May 16, 2006, and corresponding to U.S. application Ser. No. 10/719,465 filed Nov. 21, 2003; which are incorporated by reference in their entirety herein. In the case of any conflict of terminology in the context of Formula 163, these references incorporated by reference herein control.

In an embodiment, the Kv7 channel activator is a compound according to Formula 163:

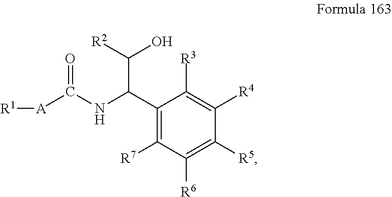

wherein, R¹ is selected from the group consisting of pyridinyl, 3-quinolinyl, 2-thienyl, furanyl, C₃₋₆ cycloalkyl and phenyl optionally substituted with substituent independently selected from the group consisting of halogen, C₁₋₄ alkyl, C₁₋₄ alkoxy, trifluoromethyl, trifluoromethoxy and nitro; A is —CH═CH— or —(CH₂)ₙ—; R² is hydrogen or hydroxymethyl; n is an integer of 0, 1 or 2; R⁴ is selected from the group consisting of di(C₁₋₄ alkyl)amino, trifluoromethoxy and optionally substituted morpholin-4-yl, morpholin-4-ylmethyl, pyridinyl, pyrimidinyl, piperazinyl, and pyrazinyl with one or two substituents in which said substituent is independently selected from the group consisting of C₁₋₄ alkyl, aminomethyl, hydroxymethyl, chloro or fluoro; R₅ is hydrogen or fluoro; or R⁴ and R⁵ taken together is —CH═CH—CH═CH-optionally substituted with a substituent independently selected from the group consisting of C₁₋₄ alkyl, C₁₋₄ alkoxy, trifluoromethyl and trifluoromethoxy; and R³, R⁶, and R⁷ are each independently hydrogen or fluoro.

### Formula 164

In an embodiment, the Kv7 channel activator may be selected from one of the following compounds according to Formula 164. Such compounds are described in International Publication No. WO2004047743A2, published Jun. 10, 2004, and corresponding to International Application No. PCT/US2003/037348 filed Nov. 21, 2003; U.S. Pat. No. 7,045,551 issued May 16, 2006, and corresponding to U.S. application Ser. No. 10/719,465 filed Nov. 21, 2003; which are incorporated by reference in their entirety herein. In the case of any conflict of terminology in the context of Formula 164, these references incorporated by reference herein control.

In an embodiment, the Kv7 channel activator is a compound according to Formula 164:

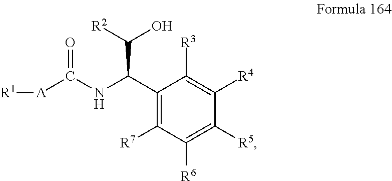

wherein, R¹ is selected from the group consisting of pyridinyl, 3-quinolinyl, 2-thienyl, furanyl, C₃₋₆ cycloalkyl and phenyl optionally substituted with substituent independently selected from the group consisting of halogen, C₁₋₄ alkyl, C₁₋₄ alkoxy, trifluoromethyl, trifluoromethoxy and nitro; A is —CH═CH— or —(CH₂)ₙ-; R² is hydrogen; n is an integer of 0, 1 or 2; R⁴ is selected from the group consisting of di(C₁₋₄ alkyl)amino, trifluoromethoxy and optionally substituted morpholin-4-yl, morpholin-4-ylmethyl, pyridinyl, pyrimidinyl, piperazinyl, and pyrazinyl with one or two substituents in which said substituent is independently selected from the group consisting of C₁₋₄ alkyl, aminomethyl, hydroxymethyl, chloro or fluoro; R⁵ hydrogen or fluoro; or R⁴ and R⁵ taken together is —CH═CH—CH═CH— optionally substituted with a substituent independently selected from the group consisting of C₁₋₄ alkyl, C₁₋₄ alkoxy, trifluoromethyl and trifluoromethoxy; and R³, R⁶, and R⁷ are each independently hydrogen or fluoro.

In further embodiments, the Kv7 Channel activator is selected from the group consisting of: (R)—N-[2-hydroxy-1-(3-morpholin-4-yl-phenyl)-ethyl]-3-phenyl-propionamide; (R)-3-(2-fluoro-phenyl)-N-[2-hydroxy-1-(3-morpholin-4-yl-phenyl)-ethyl]-acrylamide; (R)-3-(3-fluoro-phenyl)-N-[2-hydroxy-1-(3-morpholin-4-yl-phenyl)-ethyl]-acrylamide; (R)-3-(2,4-difluoro-phenyl)-N-[2-hydroxy-1-(3-morpholin-4-yl-phenyl)-ethyl]-acrylamide; (R)—N-[1-(4-fluoro-3-morpholin-4-yl-phenyl)-2-hydroxy-ethyl]-3-(2-fluoro-phenyl)-acrylamide (R)—N-[1-(4-fluoro-3-morpholin-4-yl-phenyl)-2-hydroxy-ethyl]-3-(3-fluoro-phenyl)-acrylamide (R)—N-[1-(4-fluoro-3-morpholin-4-yl-phenyl)-2-hydroxy-ethyl]-3-(4-fluoro-phenyl)-acrylamide (R)-3-(2,4-difluoro-phenyl)-N-[1-(4-fluoro-3-morpholin-4-yl-phenyl)-2-hydroxy-ethyl]-acrylamide (R)-3-(3-fluoro-phenyl)-N-(2-hydroxy-1-naphthalen-2-yl-ethyl)-acrylamide; (R)-3-(4-fluoro-phenyl)-N-(2-hydroxy-1-naphthalen-2-yl-ethyl)-acrylamide; (R)-3-(2,4-difluoro-phenyl)-N-(2-hydroxy-1-naphthalen-2-yl-ethyl)-acrylamide; (R)-3-(3,4-difluoro-phenyl)-N-(2-hydroxy-1-naphthalen-2-yl-ethyl)-acrylamide; (R)-4-fluoro-N-(2-hydroxy-1-naphthalen-2-yl-ethyl)-benzamide; (R)-2,3-difluoro-N-(2-hydroxy-1-naphthalen-2-yl-ethyl)-benzamide; (R)-2,4-difluoro-N-(2-hydroxy-1-naphthalen-2-yl-ethyl)-benzamide; (R)-3,4-difluoro-N-(2-hydroxy-1-naphthalen-2-yl-ethyl)-benzamide; (R)-2-(2,4-difluoro-phenyl)-N-(2-hydroxy-1-naphthalen-2-yl-ethyl)-acetamide; (R)-3-(2-fluoro-phenyl)-N-(2-hydroxy-1-naphthalen-2-yl-ethyl)-propionamide; (R)-3-(3-fluoro-phenyl)-N-(2-hydroxy-1-naphthalen-2-yl-ethyl)-propionamide; (R)-3-(4-fluoro-phenyl)-N-(2-hydroxy-1-naphthalen-2-yl-ethyl)-propionamide; (R)-3-(2,4-difluoro-phenyl)-N-(2-hydroxy-1-naphthalen-2-yl-ethyl)-propionamide; (R)-3-(2-fluoro-phenyl)-N-[2-hydroxy-1-(7-methoxy-naphthalen-2-yl)-ethyl]-acrylamide; (R)-3-(3-fluoro-phenyl)-N-[2-hydroxy-1-(7-methoxy-naphthalen-2-yl)-ethyl]-acrylamide; (R)-3-(4-fluoro-phenyl)-N-[2-hydroxy-1-(7-methoxy-naphthalen-2-yl)-ethyl]-acrylamide; (R)-3-(2,4-difluoro-phenyl)-N-[2-hydroxy-1-(7-methoxy-naphthalen-2-yl)-ethyl]-acrylamide; (1R, 2S)—N-(2,3-dihydroxy-1-naphthalen-2-yl-propyl)-3-(2-fluoro-phenyl)-acrylamide; (1R, 2S)-3-(2,4-difluoro-phenyl)-N-(2,3-dihydroxy-1-naphthalen-2-yl-propyl)-acrylamide; (1R, 2S)-3-(3,4-difluoro-phenyl)-N-(2,3-dihydroxy-1-naphthalen-2-yl-propyl)-acrylamide; and (1R, 2S)-3-(3,5-difluoro-phenyl)-N-(2,3-dihydroxy-1-naphthalen-2-yl-propyl)-acrylamide; or a pharmaceutically acceptable salt thereof.

### Formula 165

In an embodiment, the Kv7 channel activator may be selected from one of the following compounds according to Formula 165. Such compounds are described in U.S. Pat. No. 7,135,472 issued Nov. 14, 2006, and corresponding to U.S. application Ser. No. 10/719,187 filed Nov. 21, 2003; International Publication No. WO2004047744A2, published Jun. 10, 2004, and corresponding to International Application No. PCT/US2003/037349 filed Nov. 21, 2003; U.S. Pat. No. 7,135,472 issued Nov. 14, 2006, and Patent Cooperation Treaty application No. US2003/037349 published Jun. 10, 2004, which are incorporated by reference in their entirety herein. In the case of any conflict of terminology in the context of Formula 165, these references incorporated by reference herein control.

In an embodiment, the Kv7 channel activator is a compound according to Formula 165:

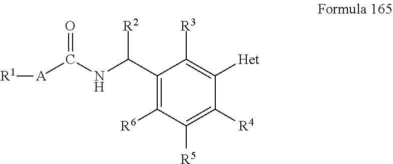

wherein, R¹ is selected from the group consisting of straight or branched chain C_{x_6} alkyl optionally substituted with amino, C alkylamino or di(C_{1.4} alkyl)amino, pyridinyl, pyrrodidinyl, piperidinyl, 2-thienyl, furanyl, imidazolyl, indenyl, benzofuran, C_{3.6} cycloalkyl and phenyl optionally substituted with substituent independently selected from the group consisting of halogen, CM alkyl, C alkoxy, trifluoromethyl, and trifiuoromethoxy; A is —CH═CH—, 1,1-cyclopropyl, or —(CH₂)ₙ-; R² is CM alkyl, CF₃ or hydroxymethyl; R³, R⁴, R⁵ and R⁶ each are independently hydrogen or fluoro; n is an integer of 0 to 4, inclusive; Het is selected from the group consisting of pyridinyl, pyrimidinyl, pyrazinyl, thiazolyl, imidazolyl, isoxazolyl, oxazolyl, pyrazolyl and triazolyl optionally substituted with substituents independently selected from the group consisting of CM alkyl, halogen, amino and dimethylaminomethyl; provided that when Het is pyridinyl, pyrimidinyl or pyrazinyl, then A is not —CH═CH—.

### Formula 166

In an embodiment, the Kv7 channel activator may be selected from one of the following compounds according to Formula 166. Such compounds are described in U.S. Pat. No. 7,135,472, issued Nov. 14, 2006, and corresponding to U.S. application Ser. No. 10/719,187 filed Nov. 21, 2003; International Publication No. WO2004047744A2, published Jun. 10, 2004, and corresponding to International Application No. PCT/US2003/037349 filed Nov. 21, 2003; which are incorporated by reference in their entirety herein. In the case of any conflict of terminology in the context of Formula 166, these references incorporated by reference herein control.

In an embodiment, the Kv7 channel activator is a compound according to Formula 166:

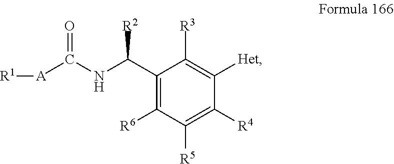

wherein, R¹ is selected from the group consisting of straight or branched chain C₁₋₆ alkyl optionally substituted with amino, C₁₋₄ alkylamino or di(C₁₋₄ alkyl)amino, pyridinyl, pyrrodidinyl, piperidinyl, 2-thienyl, furanyl, imidazolyl, indenyl, benzofuran, C₃₋₆ cycloalkyl and phenyl optionally substituted with substituent independently selected from the group consisting of halogen, C₁₋₄ alkyl, C₁₋₄ alkoxy, trifluoromethyl, and trifluoromethoxy; A is —CH═CH—, 1,1-cyclopropyl, or —(CH₂)ₙ-; R² is methyl or hydroxymethyl; R³, R⁴, R⁵ and R⁶ each are independently hydrogen or fluoro; n is an integer of 0 to 4, inclusive; Het is selected from the group consisting of pyridinyl, pyrimidinyl, pyrazinyl, thiazolyl, imidazolyl, isoxazolyl, oxazolyl, pyrazolyl and triazolyl optionally substituted with substituents independently selected from the group consisting of C₁₋₄ alkyl, halogen, amino and dimethylaminomethyl; provided that when Het is pyridinyl, pyrimidinyl or pyrazinyl, then A is not —CH═CH—.

In further embodiments, the Kv7 Channel activator is selected from the group consisting of: (S)-3-(2-fluoro-phenyl)-N-[1-(3-[1,2,4]triazol-1-yl-phenyl)-ethyl]-acrylamide; (S)-3-(2-fluoro-phenyl)-N-[1-(3-thiazol-2-yl-phenyl)-ethyl]-acrylamide; (S)-3-(2-fluoro-phenyl)-N-[1-(3-pyrazol-1-yl-phenyl)-ethyl]-acrylamide; (S)-3-(2-fluoro-phenyl)-N-[1-(3-imidazol-1-yl-phenyl)-ethyl]-acrylamide; (S)-4-phenyl-N-[1-(3-pyridin-3-yl-phenyl)-ethyl]-butyramide; (S)—N-[1-(3-pyridin-3-yl-phenyl)-ethyl]-benzamide; (S)-1H-imidazole-4-carboxylic acid [1-(3-pyridin-3-yl-phenyl)-ethyl]-amide; (S)—N-[1-(3-imidazol-1-yl-phenyl)-ethyl]-3-phenyl-acrylamide; (S)—N-[1-(3-oxazol-5-yl-phenyl)-ethyl]-3-phenyl-acrylamide; (S)-3-phenyl-N-[1-(3-thiazol-2-yl-phenyl)-ethyl]-acrylamide; (S)-3-phenyl-N-[1-(3-pyrazol-1-yl-phenyl)-ethyl]-acrylamide; and(S)-benzofuran-2-carboxylic acid {1-[3-(6-fluoro-pyridin-3-yl)-phenyl]-ethyl}-amide; or a pharmaceutically acceptable salt thereof.

### Formula 167

In an embodiment, the Kv7 channel activator may be selected from one of the following compounds according to Formula 167. Such compounds are described in International Publication No. WO2002096858A1, published Dec. 5, 2002, and corresponding to International Application No. PCT/US2002/017049 filed May 31, 2002; U.S. Pat. No. 6,831,080, issued Dec. 14, 2004, and corresponding to U.S. application Ser. No. 10/160,582 filed May 31, 2002; which are incorporated by reference in their entirety herein. In the case of any conflict of terminology in the context of Formula 167, these references incorporated by reference herein control.

In an embodiment, the Kv7 channel activator is a compound according to Formula 167:

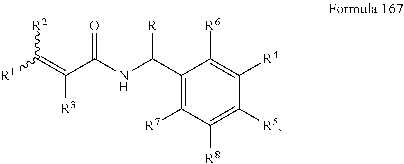

wherein, R is C₁₋₄ alkyl or trifluoromethyl; R¹ is selected from the group consisting of pyridinyl, quinolinyl, thienyl, furanyl, 1,4-benzodioxanyl, 1,3-benzodioxolyl, chromanyl, indanyl, biphenylyl, phenyl and substituted phenyl, in which said substituted phenyl is substituted with substituent independently selected from the group consisting of halogen, C₁₋₄ alkyl, C₁₋₄ alkoxy, trifluoromethyl, trifluoromethoxy and nitro; R² and R³ are each independently selected from the group consisting of hydrogen, C₁₋₄ alkyl and halogen; R⁴ is selected from the group consisting of di(C₁₋₄ alkyl)amino, trifluoromethoxy and optionally substituted morpholin-4-yl, pyridinyl, pyrimidinyl, piperazinyl, and pyrazinyl with one or two substituents in which said substituent is independently selected from the group consisting of C₁₋₄alkyl, aminomethyl, hydroxymethyl, chloro or fluoro; R⁵ is hydrogen, chloro or fluoro; or R⁴ and R⁵ taken together are —CH═CH—CH═CH— or —X(CH₂)ₘY— in which X and Y are each independently selected from the group consisting of CH₂, (CH₂)ₙN(R⁹)— and O, wherein m is 1 or 2; n is 0 or 1; R⁶, R⁷, and R⁸ are each independently selected from hydrogen, chloro and fluoro; and R⁹ is selected from the group consisting of hydrogen, C₁₋₄ alkyl, hydroxyethyl, C₁₋₄ alkoxyethyl, cyclopropylmethyl, —CO₂(C₁₋₄alkyl), and —CH₂CH₂NR¹⁰R¹¹ in which R¹⁰ and R¹¹ are each independently hydrogen or C₁₋₄ alkyl.

### Formula 168

In another embodiment, the Kv7 channel activator may be selected from one of the following compounds according to Formula 168. Such compounds are described in International Publication No. WO2002096858A1, published Dec. 5, 2002, and corresponding to International Application No. PCT/US2002/017049 filed May 31, 2002; U.S. Pat. No. 6,831,080 issued Dec. 14, 2004, and corresponding to U.S. application Ser. No. 10/160,582 filed May 31, 2002; which are incorporated by reference in their entirety herein. In the case of any conflict of terminology in the context of Formula 168, these references incorporated by reference herein control.

In an embodiment, the Kv7 channel activator is a compound according to Formula 168:

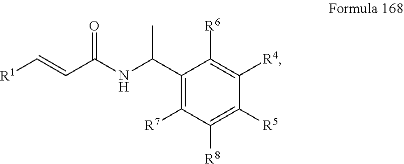

wherein, R¹ is selected from the group consisting of pyridinyl, 3-quinolinyl, 2-thienyl, benzodioxanyl, 1,3-benzodioxol-5-yl, chroman-5-yl, indan-5-yl, 4-biphenylyl, phenyl and substituted phenyl, in which said substituted phenyl is substituted with substituent independently selected from the group consisting of halogen, C₁₋₄ alkyl, C-4 alkoxy, trifluoromethyl, trifluoromethoxy and nitro; R⁴ is selected from the group consisting of optionally substituted di(C₁₋₄ alkyl)amino, trifluoromethoxy and optionally substituted morpholin-4-yl, pyridinyl, pyrimidinyl, piperazinyl, and pyrazinyl with one or two substituents in which said substituent is independently selected from the group consisting of C₁₋₄ alkyl, aminomethyl, hydroxymethyl, chloro or fluoro; R⁵ is hydrogen or fluoro; or R⁴ and R⁵ taken together are —CH═CH—CH═CH— or —X(CH₂)ₘY—, in which X and Y are each independently selected from the group consisting of CH₂, (CH₂)ₙN(R⁹)— and O, wherein m is 1 or 2; n is 0 or 1; R⁶, R⁷, and R⁸ are each independently selected from hydrogen, chloro and fluoro; and R⁹ is selected from the group consisting of hydrogen, C₁₋₄ alkyl, hydroxyethyl, C₁₋₄ alkoxyethyl, cyclopropylmethyl, —CO₂(C₁₋₄alkyl), and —CH₂CH₂NR¹⁰R¹¹ in which R¹⁰ and R¹¹ are each independently hydrogen or C₁₋₄ alkyl.

In further embodiments, R¹ is substituted phenyl or 1,3-benzodioxol-5-yl in which said substituted phenyl is substituted with one or two substituents each independently selected from the group consisting of halogen, C₁₋₄ alkyl, and C₁₋₄ alkoxy; and R⁴ and R⁵ taken together are —X(CH₂)ₘY— in which X and Y are each O, and mis 1.

In further embodiments, R¹ is selected from the group consisting of substituted phenyl, 1,3-benzodioxol-5-yl, and indan-5-yl in which said substituted phenyl is substituted with one or two substituents each independently selected from the group consisting of halogen, C₁₋₄ alkyl, C₁₋₄ alkoxy, trifluoromethoxy and trifluoromethyl; and R⁴ and R⁵ taken together are —X(CH₂)ₘY— in which X is CH₂, Y is O, and mis 1.

In further embodiments, R¹ is thienyl, phenyl or substituted phenyl in which said substituted phenyl is substituted with one or two substituents each independently selected from the group consisting of halogen, C₁₋₄ alkyl, C₁₋₄ alkoxy, trifluoromethoxy, trifluoromethyl and nitro; R⁴ is optionally substituted morpholin-4-yl with one or two substituents each independently selected from the group consisting of C₁₋₄ alkyl, aminomethyl, hydroxymethyl, chloro or fluoro; and R⁵ is hydrogen or fluoro.

In further embodiments, R¹ is phenyl, fluorophenyl or difluorophenyl.

In further embodiments, R¹ is substituted phenyl or 1,3-benzodioxol-5-yl in which said substituted phenyl is substituted with one or two substituents each independently selected from halogen or C₁₋₄ alkyl; and R⁴ and R⁵ taken together are —X(CH₂)ₘY— in which X and Y are O, and m is 2.

In further embodiments, R¹ is phenyl or substituted phenyl in which said substituted phenyl is substituted with one or two substituents each independently selected from halogen or C₁₋₄ alkyl; R⁴ and R⁵ taken together are —X(CH₂)ₘY— in which X is (CH₂)ₙN(R⁹); Y is CH₂, and m and n are 1; and R⁹ is CO2 (C₁₋₄alkyl).

In further embodiments, R¹ is substituted phenyl in which said substituted phenyl is substituted with one or two substituents each independently selected from halogen; R⁴ and R⁵ taken together are —X(CH₂)ₘY— in which X is (CH₂)ₙN(R⁹)— and Y is O wherein m is 2 and n is 0; and R⁹ is hydrogen, cyclopropylmethyl or C₁₋₄alkyl.

In further embodiments, R¹ is 3-quinolinyl or pyridinyl; R⁴ is trifluoromethoxy; and R⁵ is hydrogen.

In further embodiments, R¹ is substituted phenyl in which said substituted phenyl is substituted with one or two substituents selected from halogen or C₁₋₄alkyl; R⁴ and R⁵ taken together are —X(CH₂)ₘY—, in which X is CH₂ and Y is (CH₂)ₙN(R⁹)— wherein m is 1 and n is 0; and R⁹ is CO2 (C₁₋₄alkyl).

In further embodiments, R¹ is phenyl or substituted phenyl in which said substituted phenyl is substituted with one or two substituents selected from halogen; R⁴ and R⁵ taken together are —X(CH₂)ₘY—, in which X is (CH₂)ₙN(R⁹)— and Y is CH₂ wherein m is 2 and n is 0; and R⁹ is hydrogen, C₁₋₄alkyl, acetyl, hydroxyethyl or methoxyethyl.

In further embodiments, R¹ is phenyl or substituted phenyl in which said substituted phenyl is substituted with one or two substituents selected from halogen; R⁴ and R⁵ taken together are —X(CH₂)ₙY—, in which X is CH₂ and Y is (CH₂)ₙN(R⁹)— wherein m is 2 and n is 0; and R⁹ is hydrogen, C₁₋₄alkyl, acetyl, hydroxyethyl or methoxyethyl.

In further embodiments, R¹ is pyridinyl, phenyl or substituted phenyl in which said substituted phenyl is substituted with one or two substituents selected from halogen; R⁴ is optionally substituted pyridinyl with one or two substituents each independently selected from C₁₋₄ alkyl and halogen; and R⁵ is hydrogen or fluoro.

In further embodiments, R¹ is 1,3-benzodioxol-5-yl; R⁴ is di(C₁₋₄alkyl)amino; and R⁵ is hydrogen or fluoro.

In further embodiments, R¹ is phenyl or substituted phenyl in which said substituted phenyl is substituted with one or two substituents selected from halogen; R⁴ is pyrimidinyl; and R⁵ is hydrogen or fluoro.

In further embodiments, R¹ is phenyl or substituted phenyl in which said substituted phenyl is substituted with one or two substituents selected from halogen; R⁴ is pyrazinyl; and R⁵ is hydrogen or fluoro.

In further embodiments, R¹ is thienyl, phenyl or substituted phenyl in which said substituted phenyl is substituted with one or two substituents selected from C₁₋₄alkyl and halogen; R⁴ is piperazinyl or 4-methylpiperazinyl; and R⁵ is hydrogen or fluoro.

In further embodiments, the Kv7 Channel activator is selected from the group consisting of: 2-Methyl-3-phenyl-but-2-enoic acid (1-naphthalen-2-ylethyl)-amide; N-(1-Benzo[1,3]dioxol-5-yl-ethyl)-3-(3-methoxy-phenyl)-acrylamide; N-[1-(2,3-Dihydrobenzofuran-5-yl)ethyl]-3-(3-methoxyphenyl)-acrylamide; (S)-3-Phenyl-N-[1-(3-morpholin-4-yl-phenyl)ethyl]acrylamide; 3-(3-Fluorophenyl)-N-[1-(2,3-dihydrobenzo[1,4]dioxin-6-yl)-ethyl]acrylamide; (±)-7-{1-[3-(4-Fluorophenyl) acryloylamino]ethyl}-3,4-dihydro-1H-isoquinoline-2-carboxylic acid methyl ester; 3-(2-Fluorophenyl)-N-[1-(4-methyl-3,4-dihydro-2H-benzo[1,4]oxazin-6-yl)ethyl]-acrylamide; (S)—N-(1-Naphthalen-2-yl-ethyl)-3-phenyl-acrylamide; (S)-3-(4-Fluoro-phenyl)-N-(1-naphthalen-2-yl-ethyl)-acrylamide; (±)-N-(1-Benzo[1,3]dioxol-5-yl-ethyl)-3-(2,4-difluoro-phenyl)-acrylamide; (±)-N-[1-(2,3-Dihydro-benzofuran-5-yl)-ethyl]-3-(2-fluoro-phenyl)-acrylamide; (±)-3-(2,4-Difluoro-phenyl)-N-[1-(2,3-dihydro-benzofuran-5-yl)-ethyl]-acrylamide; (S)-3-(2,4-Difluoro-phenyl)-N-[1-(3-morpholin-4-yl-phenyl)-ethyl]-acrylamide; (S)—N-[1-(3-(2,6-Dimethyl-morpholin)-4-yl-phenyl)-ethyl]-3-phenyl-acrylamide; (S)-3-(2-Fluoro-phenyl)-N-[1-(3-morpholin-4-yl-phenyl)-ethyl]-acrylamide; (S)—N-[1-(3-Morpholin-4-yl-phenyl)-ethyl]-3-thiophen-3-yl-acrylamide; (S)-3-(4-Fluoro-phenyl)-N-[1-(3-morpholin-4-yl-phenyl)-ethyl]-acrylamide; (S)—N-{1-[3-(cis-2,6-Dimethyl-morpholin-4-yl)-phenyl]-ethyl}-3-(4-fluoro-phenyl)-acrylamide; (S)-3-(2,4-Difluoro-phenyl)-N-{1-[3-(cis-2,6-dimethyl-morpholin-4-yl)-phenyl]-ethyl}-acrylamide; (S)-3-(3,4-Difluoro-phenyl)-N-{1-[3-(cis-2,6-dimethyl-morpholin-4-yl)-phenyl]-ethyl}-acrylamide; (S)-3-(2,5-Difluoro-phenyl)-N-{1-[3-(cis-2,6-dimethyl-morpholin-4-yl)-phenyl]-ethyl}-acrylamide; (S)-3-(2-Fluoro-phenyl)-N-{1-[3-(2-methyl-morpholin-4-yl)-phenyl]-ethyl}-acrylamide; (S)-3-(3-Fluoro-phenyl)-N-{1-[3-(2-methyl-morpholin-4-yl)-phenyl]-ethyl}-acrylamide; (S)-3-(4-Fluoro-phenyl)-N-{1-[3-(2-methyl-morpholin-4-yl)-phenyl]-ethyl}-acrylamide; (S)-3-(2,4-Difluoro-phenyl)-N-{1-[3-(2-methyl-morpholin-4-yl)-phenyl]-ethyl}-acrylamide; (S)—N-{1-[3-(2-Oxa-5-aza-bicyclo[2.2.1]hept-5-yl)phenyl]ethyl}-3-phenyl-acrylamide; (S)—N-{1-[3-(2-Hydroxymethyl-morpholin-4-yl)-phenyl]-ethyl}-3-phenyl-acrylamide; (±)-N-[1-(3-Morpholin-4-yl-phenyl)-propyl]-3-phenyl-acrylamide; (±)-3-(2,4-Difluoro-phenyl)-N-[1-(3-morpholin-4-yl-phenyl)-propyl]-acrylamide; (±)-3-(2-Fluoro-phenyl)-N-[1-(3-morpholin-4-yl-phenyl)-propyl]-acrylamide; (±)-3-(3-Fluoro-phenyl)-N-[1-(3-morpholin-4-yl-phenyl)-propyl]-acrylamide; (±)-N-[1-(4-Fluoro-3-morpholin-4-yl-phenyl)-ethyl]-3-(2-fluoro-phenyl)-acrylamide; (±)-N-[1-(4-Fluoro-3-morpholin-4-yl-phenyl)-ethyl]-3-(4-fluoro-phenyl)-acrylamide; (±)-3-(2,4-Difluoro-phenyl)-N-[1-(4-fluoro-3-morpholin-4-yl-phenyl)-ethyl]-acrylamide; (±)-N-[1-(4-Fluoro-3-morpholin-4-yl-phenyl)-ethyl]-3-(4-fluoro-phenyl)-acrylamide; (±)-3-(3,4-Difluoro-phenyl)-N-[1-(4-fluoro-3-morpholin-4-yl-phenyl)-ethyl]-acrylamide; (±)-3-(2,5-Difluoro-phenyl)-N-[1-(4-fluoro-3-morpholin-4-yl-phenyl)-ethyl]-acrylamide; (±)-N-[1-(4-Fluoro-3-morpholin-4-yl-phenyl)-ethyl]-3-(3-fluoro-phenyl)-acrylamide; (±)-N-[1-(4-Fluoro-3-morpholin-4-yl-phenyl)-ethyl]-3-(2-fluoro-phenyl)-acrylamide; (±)-3-(3-Fluoro-phenyl)-N-[1-(1,2,3,4-tetrahydro-quinolin-7-yl)-ethyl]-acrylamide; (±)-3-(4-Fluoro-phenyl)-N-[1-(1,2,3,4-tetrahydro-quinolin-7-yl)ethyl]-acrylamide; (±)-3-(2-Fluoro-phenyl)-N-[1-(1-methyl-1,2,3,4-tetrahydro-quinolin-7-yl)ethyl]acrylamide; (±)-N-{1-[1-(2-Hydroxy-ethyl)-1,2,3,4-tetrahydro-quinolin-7-yl]-ethyl}-3-phenyl-acrylamide; (±)-3-(2,5-Difluoro-phenyl)-N-{1-[1-(2-hydroxy-ethyl)-1,2,3,4-tetrahydro-quinolin-7-yl]-ethyl}-acrylamide; (±)-3-(3,5-Difluoro-phenyl)-N-{1-[1-(2-hydroxy-ethyl)-1,2,3,4-tetrahydro-quinolin-6-yl]-ethyl}-acrylamide; (S)-3-Phenyl-N-[1-(3-pyridyl-phenyl)-ethyl]acrylamide; (S)-(2,4-Difluoro-phenyl)-N-[1-(3-pyridin-3-yl-phenyl)-ethyl]-acrylamide; (S)-3-Phenyl-N-[1-(3-pyridin-4-yl-phenyl)-ethyl]-acrylamide; (S)—N-{1-[3-(6-Chloro-pyridin-3-yl)-phenyl]-ethyl}-3-(2-fluoro-phenyl)-acrylamide; (S)-3-Phenyl-N-[1-(3-pyrimidin-5-yl-phenyl)-ethyl]-acrylamide; (S)-3-Phenyl-N-[1-(3-pyridin-2-yl-phenyl)-ethyl]-acrylamide; (S)-3-(2-Fluoro-phenyl)-N-[1-(3-pyridin-2-yl-phenyl)-ethyl]-acrylamide; (S)-3-(2-Fluoro-phenyl)-N-{1-[3-(6-fluoro-pyridin-3-yl)-phenyl]ethyl}-acrylamide; (S)-3-(4-Fluoro-phenyl)-N-{1-[3-(6-fluoro-pyridin-3-yl)-phenyl]-ethyl}-acrylamide; (S)—N-{1-[3-(6-Fluoro-pyridin-3-yl)-phenyl]-ethyl}-3-pyridin-3-yl-acrylamide; (S)—N-{1-[3-(6-Fluoro-pyridin-3-yl)-phenyl]-ethyl}-3-pyridin-4-yl-acrylamide; (S)—N-{1-[3-(6-Chloro-pyridin-3-yl)-phenyl]-ethyl}-3-(3-fluoro-phenyl)-acrylamide; (S)—N-{1-[3-(6-Chloro-pyridin-3-yl)-phenyl]-ethyl}-3-pyridin-3-yl-acrylamide; (S)—N-{1-[3-(6-Chloro-pyridin-3-yl)-phenyl]-ethyl}-3-pyridin-2-yl-acrylamide; (S)—N-{1-[3-(6-Chloro-pyridin-3-yl)-phenyl]-ethyl}-3-pyridin-4-yl-acrylamide; (S)—N-{1-[3-(6-Chloro-pyridin-3-yl)-phenyl]-ethyl}-3-(2-fluoro-phenyl)-acrylamide; (S)—N-{1-[3-(6-Chloro-pyridin-3-yl)-phenyl]-ethyl}-3-(2,4-difluoro-phenyl)-acrylamide; (S)—N-{1-[3-(6-Chloro-pyridin-3-yl)-phenyl]-ethyl}-3-(4-fluoro-phenyl)-acrylamide; (S)-3-(2-Fluoro-phenyl)-N-[1-(3-pyridin-3-yl-phenyl)ethyl]acrylamide; (S)—N-{1-[3-(6-Fluoro-pyridin-3-yl)-phenyl]-ethyl}-3-phenyl-acrylamide; (S)—N-{1-[3-(6-Chloro-pyridin-3-yl)-phenyl]-ethyl}-3-phenyl-acrylamide; (S)-3-(2-Fluoro-phenyl)-N-[1-(3-pyridin-4-yl-phenyl)ethyl]acrylamide; (S)-3-(2-Fluoro-phenyl)-N-[1-(3-pyrazin-2-yl-phenyl)ethyl]acrylamide; (S)-3-(2-Fluoro-phenyl)-N-[1-(3-pyrimidin-5-yl-phenyl)ethyl]acrylamide; (S)-3-(2-Fluoro-phenyl)-N-{1-[3-(4-methyl-pyridin-3-yl)phenyl]ethyl}acrylamide; (S)-3-(4-Fluorophenyl)-N-{1-[3-(4-methylpiperazin-1-yl)phenyl]ethyl}acrylamide; and(S)-3-(2,3-Difluoro-phenyl)-N-{1-[3-(4-methyl-piperazin-1-yl)-phenyl]-ethyl}-acrylamide; or a pharmaceutically acceptable salt thereof.

### Formula 169

In another embodiment, the Kv7 channel activator may be selected from one of the following compounds according to Formula 169. Such compounds are described in International Publication No. WO2002096858A1, published Dec. 5, 2002, and corresponding to International Application No. PCT/US2002/017049 filed May 31, 2002; U.S. Pat. No. 6,831,080, issued Dec. 14, 2004, and corresponding to U.S. application Ser. No. 10/160,582 filed May 31, 2002; which are incorporated by reference in their entirety herein. In the case of any conflict of terminology in the context of Formula 169, these references incorporated by reference herein control.

In an embodiment, the Kv7 channel activator is a compound according to Formula 169:

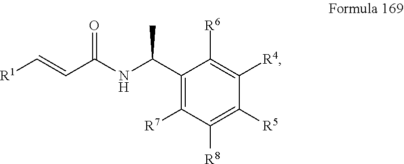

wherein, R¹ is selected from the group consisting of pyridinyl, 3-quinolinyl, 2-thienyl, benzodioxanyl, 1,3-benzodioxol-5-yl, chroman-5-yl, indan-5-yl, 4-biphenylyl, phenyl and substituted phenyl, in which said substituted phenyl is substituted with substituent independently selected from the group consisting of halogen, C₁₋₄ alkyl, C₁₋₄ alkoxy, trifluoromethyl, trifluoromethoxy and nitro; R⁴ is selected from the group consisting of optionally substituted di(C₁₋₄ alkyl)amino, trifluoromethoxy and optionally substituted morpholin-4-yl, pyridinyl, pyrimidinyl, piperazinyl, and pyrazinyl with one or two substituents in which said substituent is independently selected from the group consisting of C₁₋₄ alkyl, aminomethyl, hydroxymethyl, chloro or fluoro; R⁵ is hydrogen or fluoro; or R⁴ and R⁵ taken together are —CH═CH—CH═CH— or —X(CH₂)ₘY—, in which X and Y are each independently selected from the group consisting of CH₂, (CH₂)ₙN(R⁹)— and O, wherein m is 1 or 2; n is 0 or 1; R⁶, R⁷, and R⁸ are each independently selected from hydrogen, chloro and fluoro; and R⁹ is selected from the group consisting of hydrogen, C₄ alkyl, hydroxyethyl, C₁₋₄ alkoxyethyl, cyclopropylmethyl, —CO₂(C₁₋₄alkyl), and —CH₂CH₂NR¹⁰R¹¹ in which R¹⁰ and R¹¹ are each independently hydrogen or C₁₋₄ alkyl.

In further embodiments, R¹ is selected from the group consisting of 2-thienyl, chroman-5-yl, 4-biphenylyl, phenyl and substituted phenyl in which said substituted phenyl is substituted with one or two substituents each independently selected from the group consisting of halogen, C₁₋₄ alkyl, C₁₋₄ alkoxy, trifluoromethoxy and nitro; and R⁴ and R⁵ taken together are —CH═CH—CH═CH—.

In further embodiments, R¹ is thienyl, phenyl or substituted phenyl in which said substituted phenyl is substituted with one or two substituents each independently selected from the group consisting of halogen, C₁₋₄ alkyl, C₁₋₄ alkoxy, trifluoromethoxy, trifluoromethyl and nitro; R⁴ is optionally substituted morpholin-4-yl with one or two substituents each independently selected from the group consisting of C₁₋₄ alkyl, aminomethyl, hydroxymethyl, chloro or fluoro; and R⁵ is hydrogen or fluoro.

In further embodiments, R¹ is phenyl, fluorophenyl or difluorophenyl.

In further embodiments, R¹ is pyridinyl, phenyl or substituted phenyl in which said substituted phenyl is substituted with one or two substituents selected from halogen; R⁴ is optionally substituted pyridinyl with one or two substituents each independently selected from C₁₋₄ alkyl and halogen; and R⁵ is hydrogen or fluoro.

In further embodiments, R¹ is thienyl, phenyl or substituted phenyl in which said substituted phenyl is substituted with one or two substituents selected from C₁₋₄alkyl and halogen; R⁴ is piperazinyl or 4-methylpiperazinyl; and R⁵ is hydrogen or fluoro.

### Formula 170

In another embodiment, the Kv7 channel activator may be selected from one of the following compounds according to Formula 170. Such compounds are described in U.S. Pat. No. 6,326,385, issued Dec. 4, 2001, and corresponding to U.S. application Ser. No. 09/361,747 filed Aug. 4, 2000; International Publication No. WO2001010381A2, published Feb. 15, 2001, and corresponding to International Application No. PCT/US2000/021309 filed Aug. 4, 2000; U.S. Pat. No. 6,326,385 issued Dec. 4, 2001, and Patent Cooperation Treaty application No. US2000/021309 published Feb. 15, 2001, which are incorporated by reference in their entirety herein. In the case of any conflict of terminology in the context of Formula 170, these references incorporated by reference herein control.

In an embodiment, the Kv7 channel activator is a compound according to Formula 170:

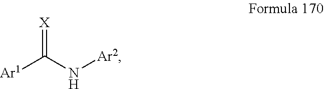

wherein, Ar¹ and Ar² are each members independently selected from the group consisting of aryl, substituted aryl, heteroaryl and substituted heteroaryl; and X is a member selected from the group consisting of O, S and N—R¹, wherein R¹ is a member selected from the group consisting of H, (C₁-C₈)alkyl, substituted (C₁-C₈)alkyl, aryl, substituted aryl, heteroaryl, substituted heteroaryl, aryl(C₁-C₄)alkyl, substituted aryl(C₁-C₄)alkyl, CN, —C(O)R², —OR³, —C(O)NR³R⁴, and —S(O)₂NR³R⁴; wherein R² is a member selected from the group consisting of (C₁-C₈)alkyl, substituted (C₁-C₈)alkyl, aryl, substituted aryl, heteroaryl, substituted heteroaryl, aryl(C₁-C₄)alkyl and substituted aryl(C₁-C₄)alkyl; and R³ and R⁴ are each members independently selected from the group consisting of hydrogen, (C₁-C₈)alkyl, substituted (C₁-C₈)alkyl, aryl, substituted aryl, heteroaryl, substituted heteroaryl, aryl(C₁-C₄)alkyl and substituted aryl(C₁-C₄)alkyl, or R³ and R⁴ can be combined with the nitrogen to which each is attached to form a 5-, 6- or 7-membered ring optionally having additional heteroatoms at the ring vertices.

In further embodiments, Ar¹ is a member selected from the group consisting of phenyl, substituted phenyl, indolyl, substituted indolyl, benzofuranyl, substituted benzofuranyl, furanyl, substituted furanyl, thienyl, substituted thienyl, isothiazolyl, substituted isothiazolyl, pyrazolyl and substituted pyrazolyl.

In further embodiments, Ar¹ is substituted phenyl, substituted or unsubstituted 2-indolyl and substituted or unsubstituted 2-thienyl.

In further embodiments, X is O.

In further embodiments, the Ar¹ substituents are selected from the group consisting of halogen, alkyl, halo(C₁-C₄)alkyl, (C₁-C₄)alkoxy, halo(C₁-C₄)alkoxy, nitro, cyano, —NHC(O)R⁷, —NHR⁷, phenyl and substituted phenyl, wherein R⁷ is a member selected from hydrogen, (C₁-C₈)alkyl, substituted (C₁-C₈)alkyl, cycloalkyl, substituted cycloalkyl, heteroalkyl, substituted heteroalkyl, heterocyclyl, substituted heterocyclyl, aryl, substituted aryl, heteroaryl, substituted heteroaryl, aryl(C₁-C₄)alkyl and substituted aryl(C₁-C₄)alkyl, or R⁷ can be combined with the nitrogen to which it is attached to form a 5-, 6- or 7-membered ring optionally having additional heteroatoms at the ring vertices.

In further embodiments, Ar² is selected from the group consisting of heteroaryl and substituted heteroaryl.

In further embodiments, Ar¹ is substituted aryl; Ar² is heteroaryl or substituted heteroaryl; and X is O.

In further embodiments, Ar² is pyridyl or substituted pyridyl.

In further embodiments, Ar² is selected from the group consisting of 6-methyl-3-pyridyl and 2-chloro-5-pyridyl.

In further embodiments, Ar¹ is substituted phenyl.

In further embodiments, the compound is according to the formula:

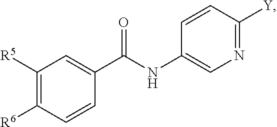

wherein, Y is a member selected from the group consisting of halogen, C₁-C₄ alkyl, C₁-C₄ substituted alkyl, —OCH₃ and —OCF₃, and R⁵ and R⁶ are members independently selected from the group consisting of H, halogen, alkyl, halo(C₁-C₄)alkyl, nitro, cyano and phenyl, with the proviso that both R⁵ and R⁶ are not H.

In further embodiments, R⁵ and R⁶ are members independently selected from the group consisting of H, F, and Cl, with the optional proviso that both R⁵ and R⁶ are not H.

Further embodiments (Collectively referred to as “Formula 171” for ease of reference in the appended claims. “Formula 171”, as recited in the claims, captures any of the below compounds preceding the NMDA receptor antagonists section.)

In another embodiment, the Kv7 channel activator may be selected from one of the following compounds. Such compounds are described in International Publication No. WO2021055538A1, published Mar. 25, 2021, and corresponding to International Application No. PCT/US2020/051171 filed Sep. 17, 2020; International Publication No. WO2017075222A1, published May 4, 2017, and corresponding to International Application No. PCT/US2016/059128 filed Oct. 27, 2016; International Publication No. WO2021211867A1, published Oct. 21, 2021, and corresponding to International Application No. PCT/US2021/027518 filed Apr. 15, 2021; International Publication No. WO2020092577A1, published May 7, 2020, and corresponding to International Application No. PCT/US2019/058878 filed Oct. 30, 2019; International Publication No. WO2021119018A1, published Jun. 17, 2021, and corresponding to International Application No. PCT/US2020/063,822 filed Dec. 8, 2020; International Publication No. WO2011085351A2, published Jul. 14, 2011, and corresponding to International Application No. PCT/US2011/020784 filed Jan. 11, 2011; US Publication No. US20100286138A1, published Nov. 11, 2010, and corresponding to U.S. application Ser. No. 12/777,935 filed May 11, 2010; US Publication No. US20210262029A1, published Aug. 26, 2021, and corresponding to U.S. application Ser. No. 17/245,734 filed Apr. 30, 2021; U.S. Pat. No. 11,221,329, issued on Jan. 11, 2022, and corresponding to U.S. application Ser. No. 15/771,857 filed Oct. 27, 2016; which are incorporated by reference in their entirety herein.

In further embodiments, the compound is selected from the group consisting of N-(1-cyclobutyl-4-fluoro-6-(1-hydroxycyclobutyl)-1H-benzo[d]imidazol-2-yl)-4,4,4-trifluoro-3,3-dimethylbutanamide; N-(1-(tert-butyl)-6-(difluoromethoxy)-1H-benzo[d]imidazol-2-yl)-3,3-dimethylbutanamide; N-(6-cyano-1-(1,1,1-trifluoro-2-methylpropan-2-yl)-1H-benzo[d]imidazol-2-yl)-3,3-dimethylbutanamide; N-(1-cyclobutyl-4-fluoro-6-(2-hydroxypropan-2-yl)-1H-benzo[d]imidazol-2-yl)-2-(1-methyl cyclopropyljacetamide; N-(6-cyano-1-cyclobutyl-4-fluoro-1H-benzo[d]imidazol-2-yl)-3,3-dimethylbutanamide; N-(1-cyclobutyl-6-fluoro-1H-benzo[d]imidazol-2-yl)-3,3-dimethylbutanamide; N-(1-(tert-butyl)-6-cyano-4-fluoro-1H-benzo[d]imidazol-2-yl)-3,3-dimethylbutanamide; N-(7-cyano-1-(1-methylcyclobutyl)-1H-benzo[d]imidazol-2-yl)-3-cyclopropyl-3-methylbutanamide; N-(6-chloro-7-cyano-1-cyclobutyl-1H-benzo[d]imidazol-2-yl)-3,3-dimethylbutanamide; N-(1-cyclobutyl-5-fluoro-1H-benzo[d]imidazol-2-yl)-3,3-dimethylbutanamide; N-(1-(tert-butyl)-6-cyano-4,7-difluoro-1H-benzo[d]imidazol-2-yl)-3,3-dimethylbutanamide; N-(6-cyano-1-cyclobutyl-7-(trifluoromethyl)-1H-benzo[d]imidazol-2-yl)-3,3-dimethylbutanamide; N-(6-cyano-4,7-difluoro-1-(1-methylcyclobutyl)-1H-benzo[d]imidazol-2-yl)-3,3-dimethylbutanamide; N-(1-(tert-butyl)-5,7-difluoro-1H-benzo[d]imidazol-2-yl)-4,4,4-trifluoro-3,3-dimethylbutanamide; and N-(1-(tert-butyl)-5,6-difluoro-1H-benzo[d]imidazol-2-yl)-2-(2,2,3,3-tetrafluorocyclobutyl) acetamide.

In another embodiment, the Kv7 channel activator is selected from the group consisting of N-(2,6-dimethyl-4-morpholin-4-yl-phenyl)-3,3-dimethyl-butyramide in a crystalline form with XRPD reflections at 10.36, 12.67, 28.64 and 29.98 (° 20) using CuK_{α1} radiation; N-(2,6-dimethyl-4-morpholin-4-yl-phenyl)-3,3-dimethyl-butyramide in a crystalline form with XRPD reflections at 8.68, 18.09, 22.60 and 30.62 (° 20) using CuK_{α1} radiation; N-(2,6-dimethyl-4-morpholin-4-yl-phenyl)-3,3-dimethyl-butyramide in a crystalline form with XRPD reflections at 8.63, 22.26, 23.40 and 30.49 (° 20) using CuK_{α1} radiation.

In further embodiments, the Kv7 Channel activator is selected from the group consisting of: Chromanol 293B, MIR-1556, UCL2077, JNJ303, L-735821 (L-7), fenofibrate; and a salt or hydrate thereof.

In further embodiments, the Kv7 Channel activator is selected from the group consisting of: a Triaminopyridine or one of its derivatives, an Acrylamide, a Benzamide, a Fenamate, a Dimethoxypyrimidine or one of its derivatives, Oxindole, Celecoxib, zinc pyrithione, ML213, QO58, Q058 lysine, NS1643, Benzbromarone, ZG1732 and ZG2083.

In further embodiments, the Kv7 Channel activator is selected from the group consisting of: NS15370, P-Retigabine, SF0034 or RL648_81 or any combination thereof.

In further embodiments, the Kv7 channel activator is selected from the group consisting of: linopirdine, (1,3-Dihydro-1-phenyl-3,3-bis(4-pyridinylmethyl)-2H-indol-2-one) XE991 (10,10-bis(4-pyridinylmethyl)-9 (10H)-anthracenone (XE991)), DMP-543 (10,10-bis(2-fluoro-4-pyridinylmethyl)-9 (10H)-anthracenone (DMP-543)), ML252 ((S)-2-phenyl-N-(2-(pyrrolidin-1-yl)phenyl) butanamide (ML252)) and UCL2077.

In further embodiments, the Kv7 channel activator is an analog of flupirtine.

In further embodiments, the Kv7 channel activator is selected from the group consisting of: N-(2,4,6-trimethylphenyl) bicyclo[2.2.1]heptane-3-carboxamide; N-(2,6-dimethylphenyl) bicyclo[2.2.1]heptane-3-carboxamide; N-(2,5-dimethylphenyl) bicyclo[2.2.1]heptane-3-carboxamide; N-(2-chloro-4,6-dimethylphenyl)pentanamide; N-(2-chloro-4,6-dimethylphenyl)-3,3-dimethylbutanamide; 2-(1-adamantyl)-N-(2,6-difluorophenyl) acetamide; N-(2-bromo-4-methylphenyl)-2-cyclopentylacetamide; N-(2,3-dimethylphenyl)-2-phenylcyclopropane-1-carboxamide; N-(4-methylphenyl)-2-phenylcyclopropane-1-carboxamide; N-(2,4-dimethoxyphenyl) bicyclo[2.2.1]heptane-3-carboxamide; 7-ethyl-1,3-dimethyl-5-(2-oxo-2-piperidin-1-ylethyl)sulfanylpyrimido[4,5-d]pyrimidine-2,4-dione; 1,3-dimethyl-7-(2-methylpropyl)-5-(2-oxo-2-piperidin-1-ylethyl)sulfanylpyrimido[4,5-d]pyrimidine-2,4-dione; 1,3-dimethyl-5-[2-(4-methylpiperidin-1-yl)-2-oxoethyl]sulfanyl-7-propylpyrimido[4,5-d]pyrimidine-2,4-dione; 7-ethyl-1,3-dimethyl-5-[2-(4-methylpiperidin-1-yl)-2-oxoethyl]sulfanylpyriinido[4,5-d]pyrimidine-2,4-dione; 2-(4-chloro-2-methyl-5-pyrrolidin-1-ylsulfonylphenoxy)-N-cyclohexylacetamide; 2-(4-chloro-2-methyl-5-thiomo holin-4-ylsulfonylphenoxy)-N-cyclohexylacetamide; 1-[1-(1,3-benzodioxol-5-yl)ethyl]-3-cyclohexylurea; 1-(1-adamantyl)-3-[1-(1,3-benzodioxol-5-yl)ethyl]urea; 3-(5-bromo-2-hydroxyphenyl)-N-(furan-2-ylmethyl)-3-phenylpropanamide; ethyl 2-benzamido-2-(2,6-dimethylpyran-4-ylidene)acetate; (2,4-dichlorophenyl)methyl 1-benzamido-5-oxopyrrolidine-3-carboxylate; N-[2-cyano-5-(cyanomethylsulfanyl)-4-phenylthiophen-3-yl]-2-phenylacetamide; N-(6-bromo-1,3-diethyl-2-oxobenzimidazol-5-yl)-2-phenylpropanamide; N-(2,5-diethoxy-4-mo holin-4-ylphenyl)-2-phenylacetamide; N-[4-[(5-methylthiophen-2-yl)methylamino]phenyl]butanamide; N-[(2-methylsulfanyl-6-propan-2-ylpyridin-3-yl)methyl]-3-phenylbutanamide; 4-bromo-N-(thiophen-2-ylmethylideneamino)-1H-pyrazole-5-carboxamide; S-[2-[(6-fiuoro-3-prop-2-ynyl-1,3-benzothiazol-2-ylidene)amino]-2-oxoethyl]ethanethioate; 1-cyclohexyl-3-[1-(3,4-dimethylphenyl)ethyl]thiourea; N-[4-methyl-1-oxo-1-(1-phenylethylamino)pentan-2-yl]cyclohexanecarboxamide; 5-chloro-2-[(3-methylphenyl)methylsulfonyl]-N-(5-propyl-I, 3,4-thiadiazol-2-yl)pyrimidine-4-carboxamide; 2-benzylsulfonyl-5-chloro-N-(5-propan-2-yl-1,3,4-thiadiazol-2-yl)pyrimidine-4-carboxamide; and 4-bromo-N-(furan-2-ylmethylideneamino)-1H-pyrazole-5-carboxamide.

In further embodiments, the metal channel activator is QRA-244 as described in Dan Elbaum et al. “TST-20: QRA-244 a Potent, Selective KCNQ2/3 Opener and a Potential Therapy for Motor System Hyperexcitability induced Disease Progression in ALS patients”; 31^{st} International symposium on ALS/MND; Live Poster Session C, Dec. 11, 2020.

### NMDA Receptor Antagonists

NMDA receptor antagonists are a class of pharmaceutically active molecules that work to antagonize, or inhibit the action of, the N-Methyl-D-aspartate receptor (NMDAR). Some examples of the NMDA receptor antagonists include ketamine, lanicemine, dextromethorphan (DXM), phencyclidine (PCP), methoxetamine (MXE), and nitrous oxide (N₂O). Several synthetic opioids function additionally as NMDA_{R}-antagonists, such as pethidine, levorphanol, methadone, dextropropoxyphene, tramadol and ketobemidone. As described herein, there are several other NMDA receptor antagonist compounds and classes of NMDA receptor antagonists

The NMDA receptor is an ionotropic receptor that allows for the transfer of electrical signals between neurons in the brain and in the spinal column. For electrical signals to pass, the NMDA receptor must be open. To remain open, glutamate and glycine must bind to the NMDA receptor. An NMDA receptor that has glycine and glutamate bound to it and has an open ion channel is called “activated.”

Chemicals that deactivate the NMDA receptor are known as NMDA receptor antagonists (NMDA_{R}). NMDA_{R} antagonists fall into four categories: competitive antagonists, which bind to and block the binding site of the neurotransmitter glutamate; glycine antagonists, which bind to and block the glycine site; noncompetitive antagonists, which inhibit NMDA_{R} by binding to allosteric sites; and uncompetitive antagonists, which block the ion channel by binding to a site within it.

In an embodiment, the NMDA receptor antagonist is ketamine or a pharmaceutically acceptable salt thereof. In another embodiment, the NMDA receptor antagonist is (S)-1-phenyl-2-(pyridin-2-yl) ethanamine (lanicemine) or a pharmaceutically acceptable salt thereof. In another embodiment, the NMDA receptor antagonist may be a prodrug of ketamine or a pharmaceutically acceptable salt thereof. In another embodiment, the NMDA receptor antagonist may be EVT-101:

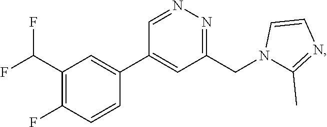 [SMILES: Cc1nccn1Cc1cc(-c2ccc(F)c(C(F)F)c2)cnn1]

or a pharmaceutically acceptable salt thereof.

In another embodiment, the NMDA receptor antagonist is rapastinel:

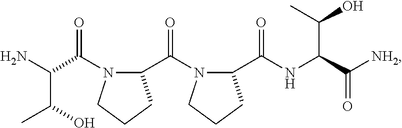

(S)—N-((2S,3R)-1-amino-3-hydroxy-1-oxobutan-2-yl)-1-((S)-1-((S)-2-amino-3-hydroxypropanoyl)pyrrolidine-2-carbonyl)pyrrolidine-2-carboxamide, (S)—N—((S)-1-amino-3-hydroxy-1-oxopropan-2-yl)-1-((S)-1-((2S,3R)-2-amino-3-hydroxybutanoyl)pyrrolidine-2-carbonyl)pyrrolidine-2-carboxamide, (S)—N—((S)-1-amino-3-hydroxy-1-oxopropan-2-yl)-1-((S)-1-((S)-2-amino-3-hydroxy-propanoyl)-pyrrolidine-2-carbonyl)-pyrrolidine-2-carboxamide, and/or N-((2S,3R)-1-amino-3-hydroxy-1-oxobutan-2-yl)-1-((S)-1-((2S,3R)-2-amino-3-hydroxybutanoyl)-pyrrolidine-2-carbonyl)-2-benzylpyrrolidine-2-carboxamide

In another embodiment, the NMDA receptor antagonist may be amantadine or a pharmaceutically acceptable salt thereof. In another embodiment, the NMDA receptor antagonist may be lanicemine. In another embodiment, the NMDA receptor antagonist may be a prodrug of lanicemine.

In further embodiments, for example, the prodrug of lanicemine may have the following chemical structure:

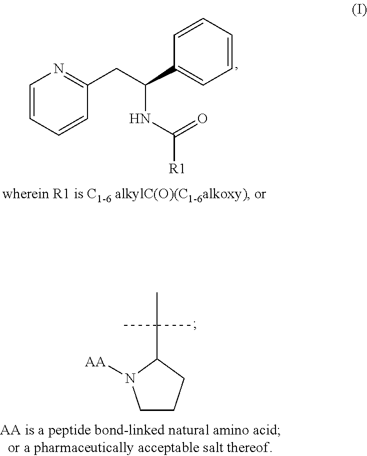

In further embodiments, the prodrug of lanicemine may have the following structure:

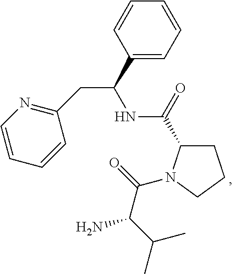 [SMILES: CC(C)[C@H](N)C(=O)N1CCC[C@H]1C(=O)N[C@@H](Cc1ccccn1)c1ccccc1]

or a pharmaceutically acceptable salt thereof.

In further embodiments, pharmaceutical compositions including the following Compound A or a pharmaceutically acceptable salt thereof and an NMDA receptor antagonist or a pharmaceutically acceptable salt thereof:

### 1. Compound A:

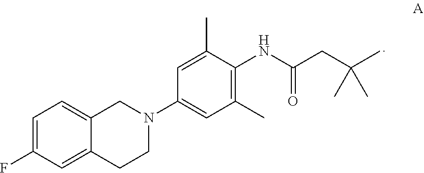 [SMILES: Cc1cc(N2CCc3cc(F)ccc3C2)cc(C)c1NC(=O)CC(C)(C)C]

The NMDA receptor antagonist may be selected from ketamine, lanicemine, dextromethorphan (DXM), phencyclidine (PCP), methoxetamine (MXE), and nitrous oxide (N2O).

In another embodiment, provided is a pharmaceutical composition including the following compound (ezogabine) or a pharmaceutically acceptable salt thereof and an NMDA receptor antagonist or a pharmaceutically acceptable salt thereof:

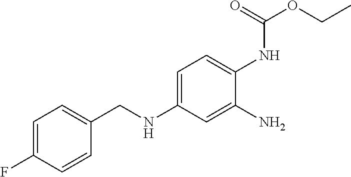 [SMILES: CCOC(=O)Nc1ccc(NCc2ccc(F)cc2)cc1N]

In another embodiment, the NMDA receptor antagonist is flupirtine:

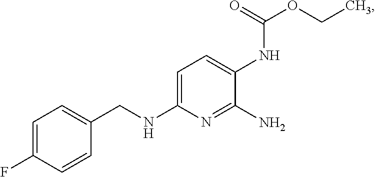 [SMILES: CCOC(=O)Nc1ccc(NCc2ccc(F)cc2)nc1N]

or a pharmaceutically acceptable salt thereof.

In an embodiment, the NMDA receptor antagonist may be selected from ketamine, lanicemine, dextromethorphan (DXM), phencyclidine (PCP), methoxetamine (MXE), and pharmaceutically acceptable salts and prodrugs thereof, or nitrous oxide (N20).

In various embodiments, the NMDA receptor antagonist is one or more of a compound according to Formulas 200-260. Pharmaceutical compositions may include one or more metal channel activators according to Formulas 1-171 and one or more NMDA receptor antagonist compounds according to Formulas 200-260 described below.

### Formula 200

In an embodiment, the NMDA receptor antagonist may be selected from one of the following compounds according to Formula 200. Such compounds are described in International Publication No. WO2015/067923, published May 14, 2015, and corresponding to International Application No. PCT/GB2014/053236 filed Oct. 30, 2014, as well as U.S. Pat. No. 9,822,075 issued on Nov. 21, 2017; U.S. Pat. No. 10,207,994 issued on Feb. 19, 2019; U.S. Pat. No. 10,815,199 issued on Oct. 27, 2020, and U.S. Pat. No. 11,358,935 issued on Jun. 14, 2022, which are each incorporated by reference in their entirety herein. In the case of any conflict of terminology of Formula 200, these references incorporated by reference herein control.

In an embodiment, the NMDA receptor antagonist is lanicemine, or a prodrug of lanicemine or a pharmaceutically acceptable salt thereof. The prodrug of lanicemine may have the following chemical structure:

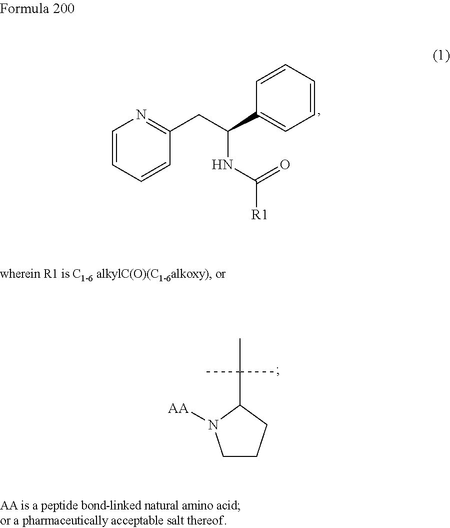

In further embodiments, for example, the prodrug of lanicemine may have the following chemical structure:

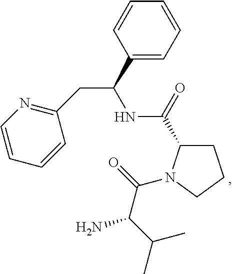 [SMILES: CC(C)[C@H](N)C(=O)N1CCC[C@H]1C(=O)N[C@@H](Cc1ccccn1)c1ccccc1]

or a pharmaceutically acceptable salt thereof.

In further embodiments, the NMDA receptor antagonist is one or more of:
(S)-(1-Phenyl-2-(pyridin-2-yl)ethylcarbamoyloxy)methyl isobutyrate;
2-Methyl-1-((S)-1-phenyl-2-(pyridin-2-yl)ethylcarbamoyloxy)propyl isobutyrate;
2-Methyl-1-((S)-1-phenyl-2-(pyridin-2-yl)ethylcarbamoyloxy)propyl isobutyrate diastereomer 1;
2-Methyl-1-((S)-1-phenyl-2-(pyridin-2-yl)ethylcarbamoyloxy)propyl isobutyrate diastereomer 2;
1-((S)-1-phenyl-2-(pyridin-2-yl)ethylcarbamoyloxy)ethyl acetate;
1-((S)-1-phenyl-2-(pyridin-2-yl)ethylcarbamoyloxy)ethyl acetate diastereomer 1; 1-((S)-1-phenyl-2-(pyridin-2-yl)ethylcarbamoyloxy)ethyl acetate diastereomer 2; 1-((S)-1-Phenyl-2-(pyridin-2-yl)ethylcarbamoyloxy)ethyl isobutyrate;
1-((S)-1-Phenyl-2-(pyridin-2-yl)ethylcarbamoyloxy)ethyl isobutyrate diastereomer 1;
1-((S)-1-Phenyl-2-(pyridin-2-yl)ethylcarbamoyloxy)ethyl isobutyrate diastereomer 2;
(SJ-i-((S)-2-Amino-3-methylbutanoyl)-N—((S)-1-phenyl-2-(pyridin-2-yl)ethyl)pyrrolidine-2-carboxamide; (S)-1-((S)-2,6-Diaminohexanoyl)-N—((S)-1-phenyl-2-(pyridin-2-yl)ethyl)pyrrolidine-2-carboxamide;
(S)-1-((S)-2-Amino-3-(1H-imidazol-4-yl)propanoyl)-N—((S)-1-phenyl-2-(pyridin-2-yl)ethyl)pyrrolidine-2-carboxamide;
2-((S)-2-((S)-1-((S)-2-Amino-3-(4-hydroxyphenyl)propanoyl)pyrrolidine-2-carboxamido)-2-phenylethyl)pyridine;
2-((S)-2-((S)-1-((S)-2-Amino-3-(1H-indol-3-yl)propanoyl)pyrrolidine-2-carboxamido)-2-phenylethyl)pyridine;
(S)-1-((S)-2-Amino-3-phenylpropanoyl)-N—((S)-1-phenyl-2-(pyridin-2-yl)ethyl)pyrrolidine-2-carboxamide; or,
(S)-1-((S)-2-Amino-4-methylpentanoyl)-N—((S)-1-phenyl-2-(pyridin-2-yl)ethyl)pyrrolidine-2-carboxamide;
   or a pharmaceutically acceptable salt of any one of the foregoing.

In further embodiments, the NMDA receptor antagonist is (S)-1-((S)-2-amino-3-methylbutanoyl)-N—((S)-1-phenyl-2-(pyridin-2-yl)ethyl)pyrrolidine-2-carboxamide or a pharmaceutically acceptable salt thereof.

In further embodiments, the NMDA receptor antagonist is:

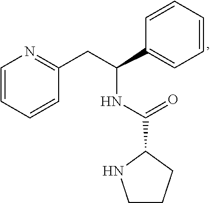 [SMILES: O=C(N[C@@H](Cc1ccccn1)c1ccccc1)[C@@H]1CCCN1]

or a pharmaceutically acceptable salt thereof.

In further embodiments, the NMDA receptor antagonist is:

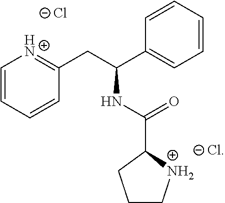

In further embodiments, the NMDA receptor antagonist is a compound according to:

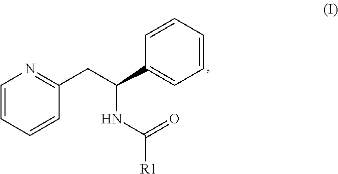

wherein R1 is C1-6 alkylC(O)O(C1-6 alkoxy), or

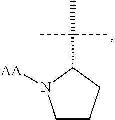

wherein AA is a peptide bond-lined natural amino acid; or a pharmaceutically acceptable salt thereof.

In further embodiments, the NMDA receptor antagonist is (S)-1-((S)-2-amino-3-methylbutanoyl)-N—((S)-1-phenyl-2-(pyridin-2-yl)ethyl)pyrrolidine-2-carboxamide dihydrochloride salt:

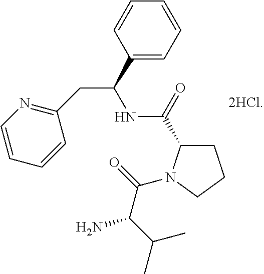 [SMILES: CC(C)[C@H](N)C(=O)N1CCC[C@H]1C(=O)N[C@@H](Cc1ccccn1)c1ccccc1]

In further embodiments, the NMDA receptor antagonist is (S)-1-((S)-2-amino-3-methylbutanoyl)-N—((S)-1-phenyl-2-(pyridin-2-yl)ethyl)pyrrolidine-2-carboxamide fumarate salt:

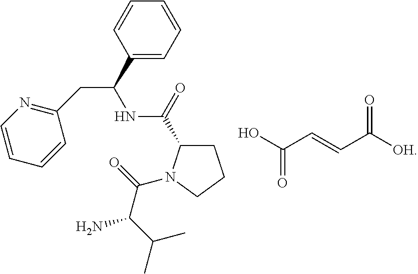

### Formula 201

In an embodiment, the NMDA receptor modulator may be selected from one of the following compounds according to Formula 201. Such compounds are described in International Publication No. WO 2021/228123, published Nov. 18, 2021 and corresponding to PCT/CN2021/093264, filed May 12, 2021, is incorporated by reference in its entirety herein. In the case of any conflict of terminology of Formula 201, these references incorporated by reference herein control.

In an embodiment, the NMDA receptor modulator is a compound according to Formula 201:

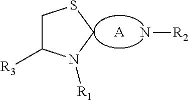

or a pharmaceutically acceptable salt or ester, stereoisomer, or solvate thereof, wherein, Ring A is 3- to 8-membered aliphatic heterocycle; R1 is selected from H, C1-C6 alkyl, aryl-C1-C4 alkyl, C2-C6 acyl, —CONRR′ and natural amino acid fragments; R2 is selected from H, C1-C6 alkyl, C1-C6 alkoxycarbonyl, C2-C6 acyl, —CONRR′ and natural amino acid fragments; R3 is selected from H, cyano, 3- to 8-membered nitrogen-containing aliphatic heterocycle-C1-C4 acyl, C1-C6 alkoxycarbonyl and —CONHR4; R and R′ are each independently selected from H and C1-C6 alkyl; R4 is selected from H, C1-C6 alkyl, natural amino acid fragments and carboxylic acid derivatives of the said natural amino acid fragments; optionally, the C1-C6 alkyl, aryl-C1-C4 alkyl, C2-C6 acyl, 3- to 8-membered nitrogen-containing aliphatic heterocycle-C1-C4 acyl, C1-C6 alkoxycarbonyl, —CONRR′, —CONHR4 and natural amino acid fragments are each independently substituted by one or more substituents selected from: halogen, amino, hydroxyl, cyano, carboxyl, nitro, C1-C6 alkyl and C1-C6 alkoxy.

In further embodiments, the NMDA receptor modulator is selected from:

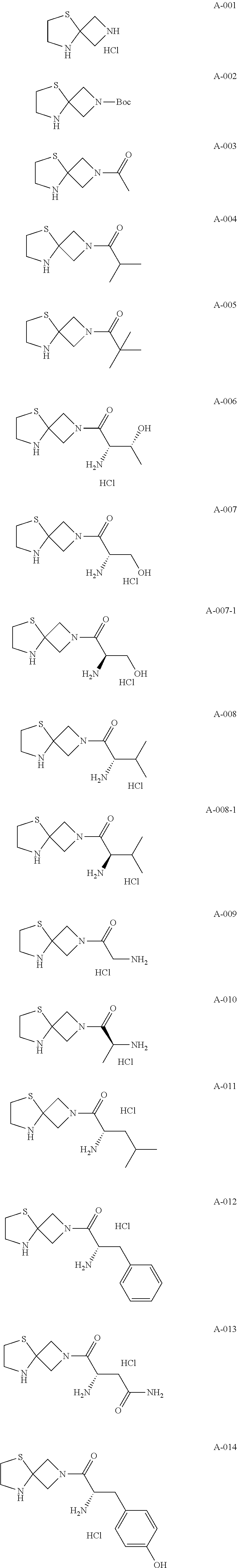

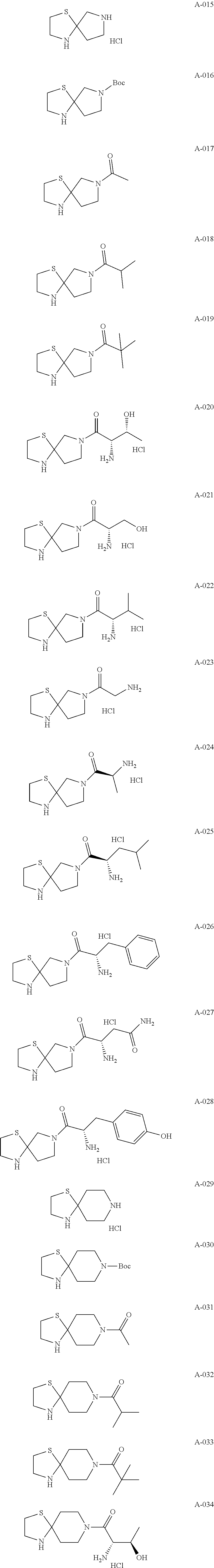

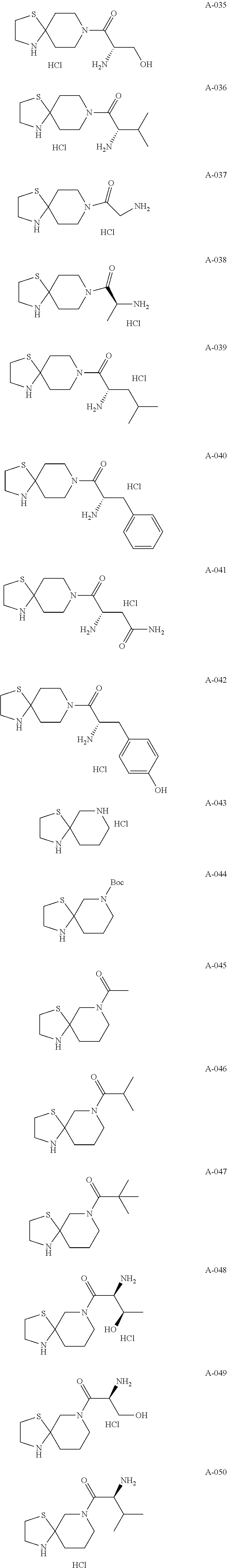

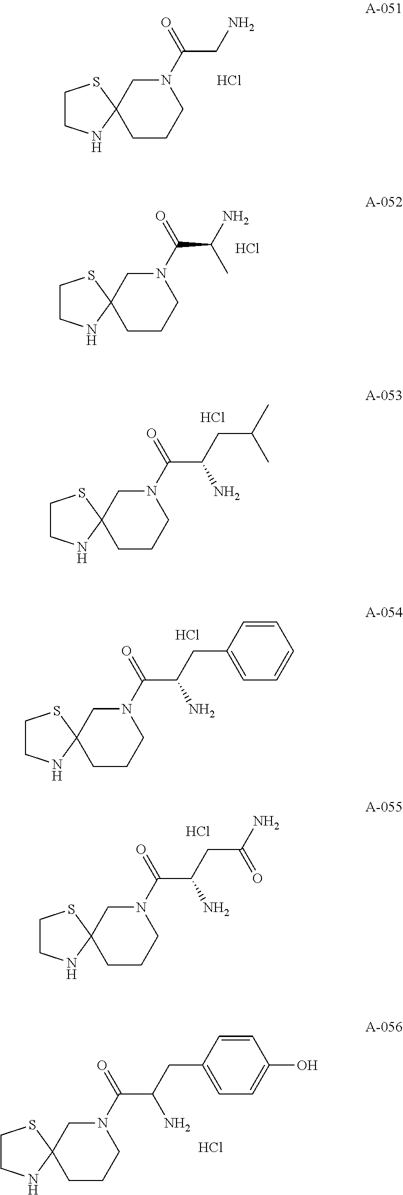

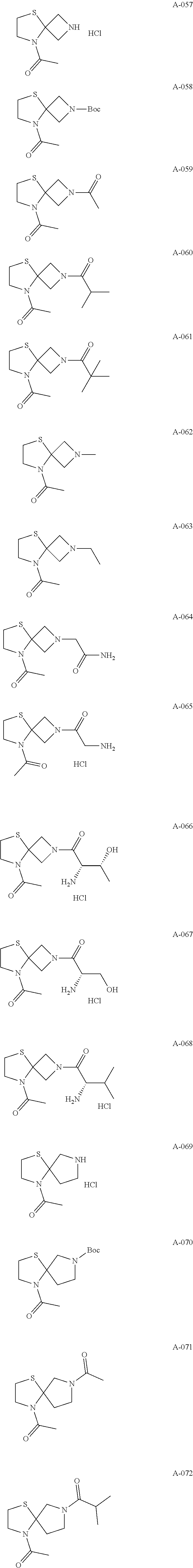

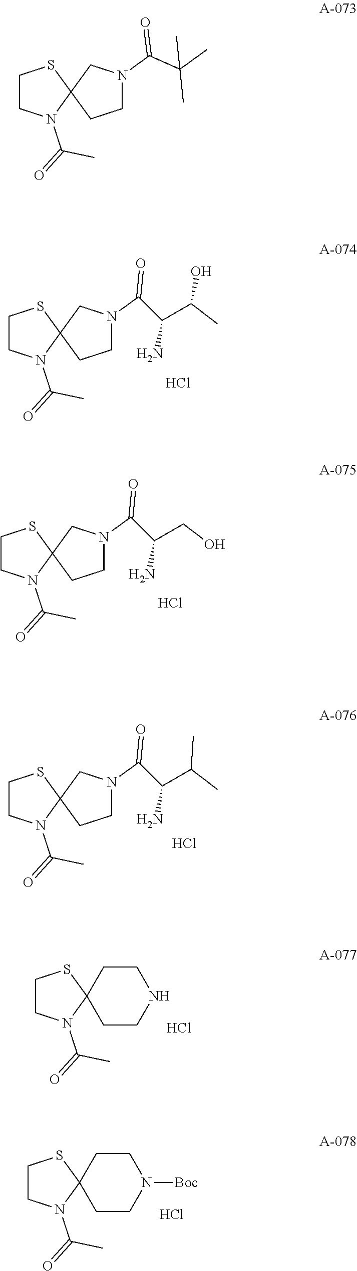

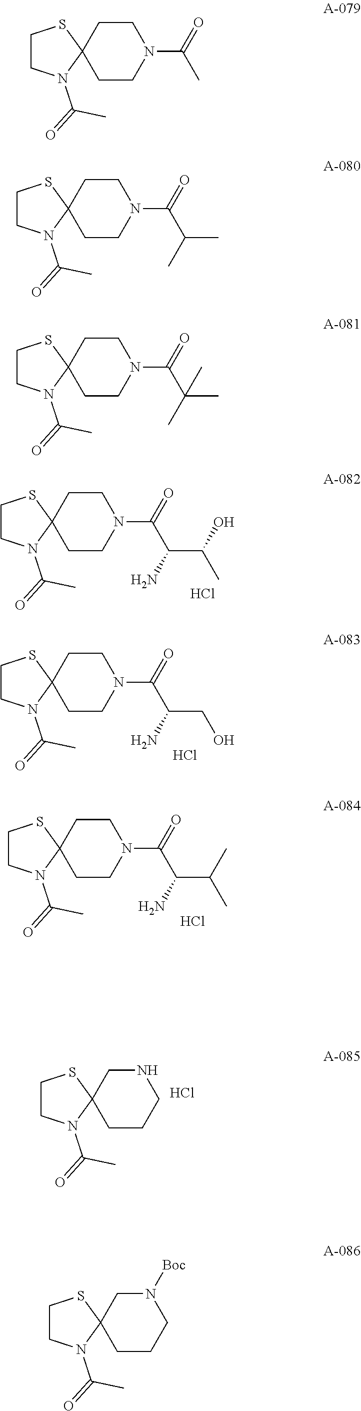

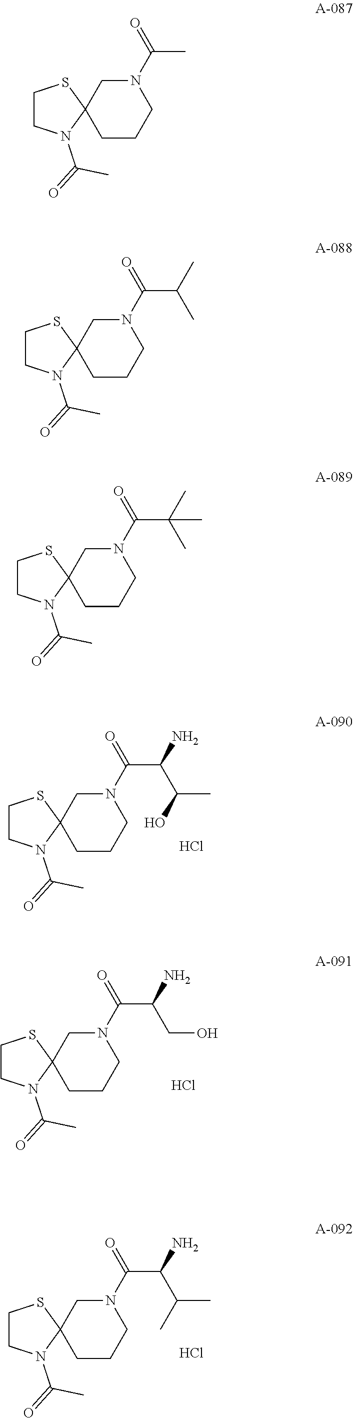

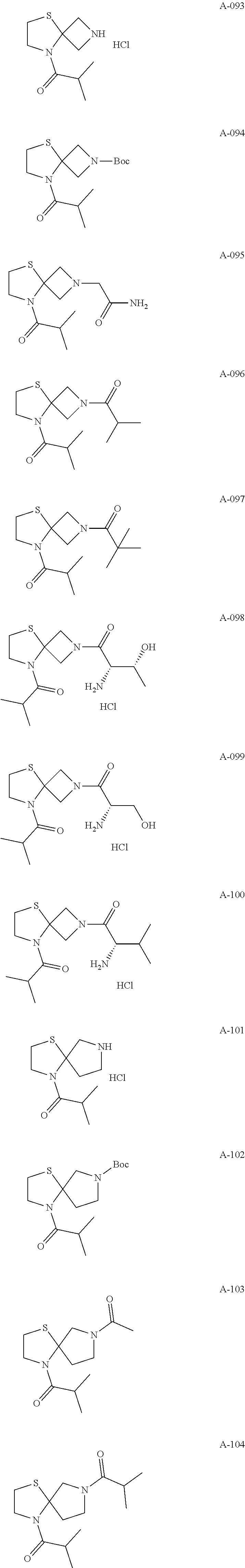

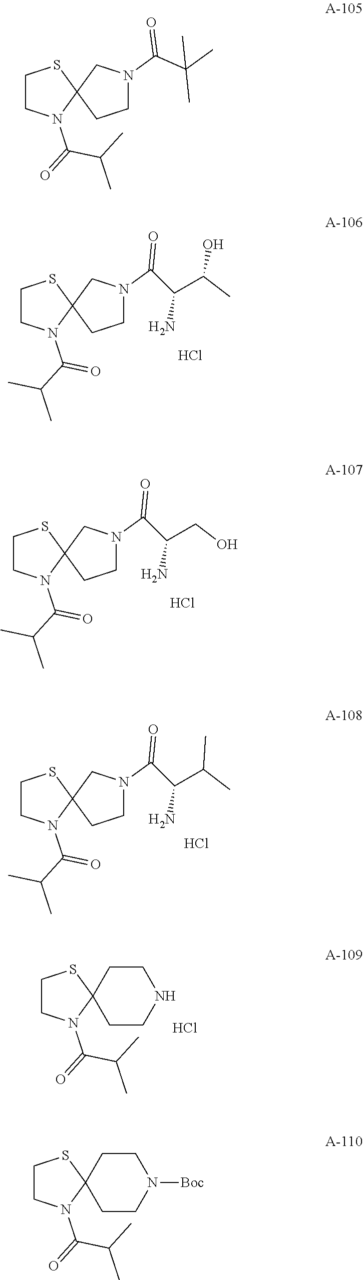

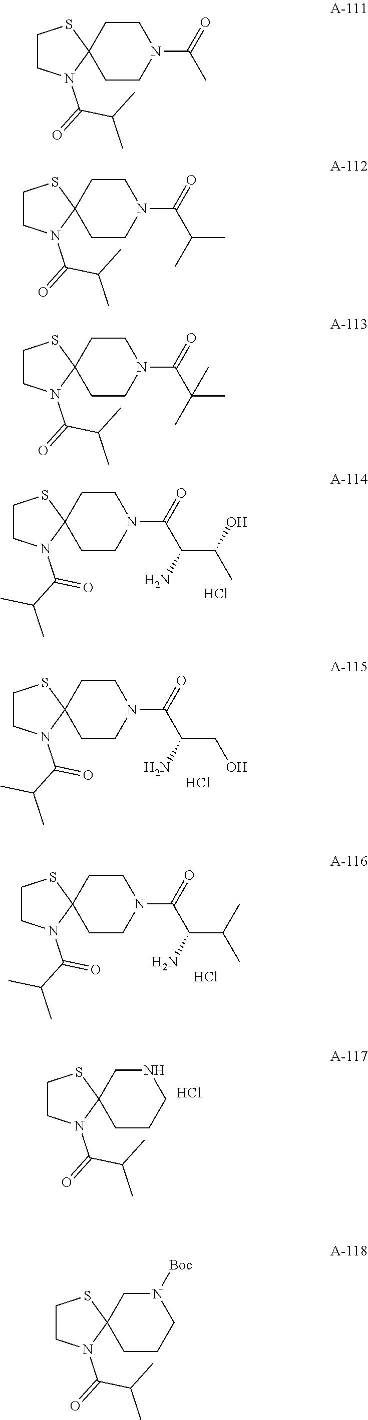

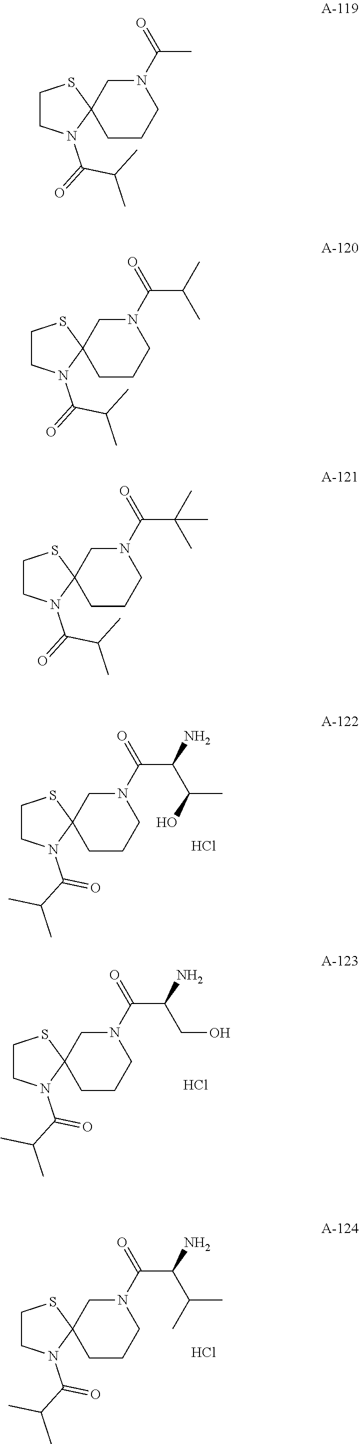

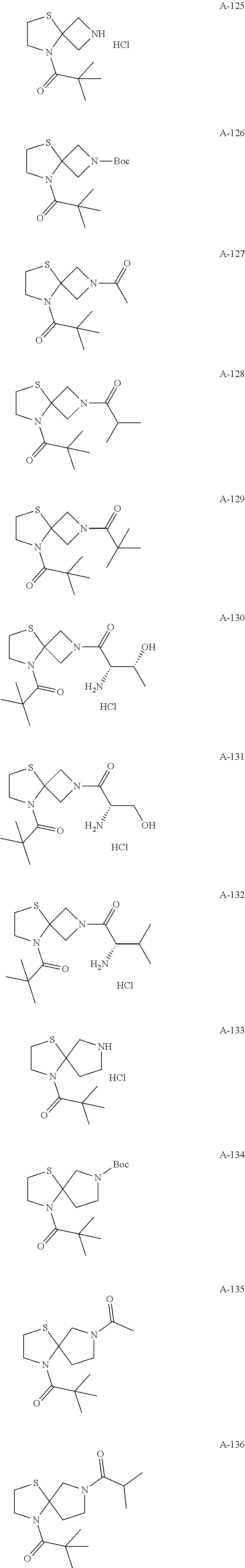

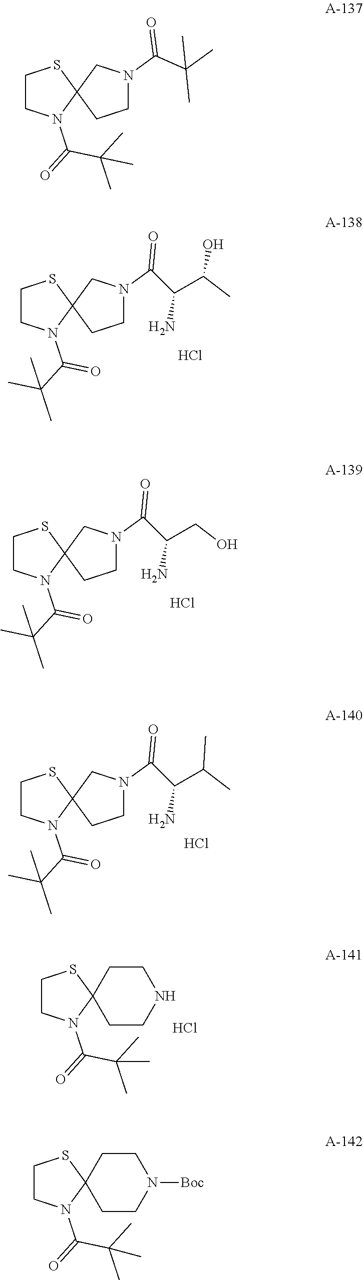

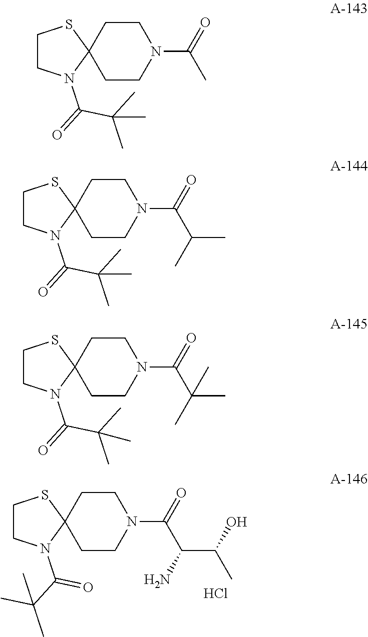

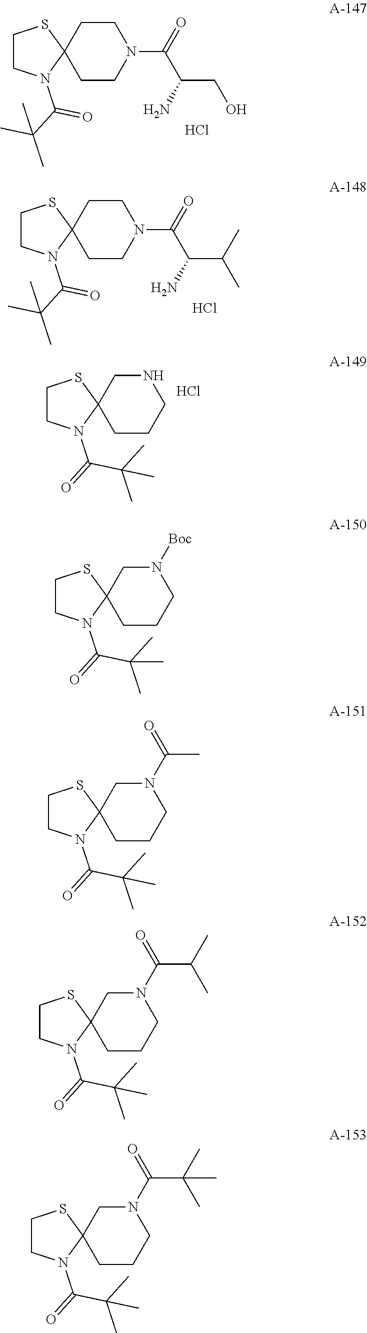

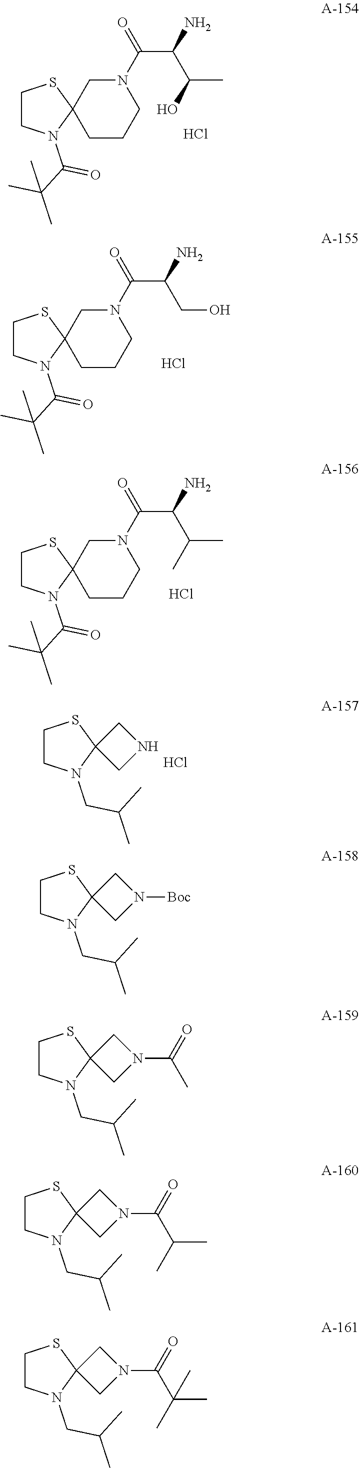

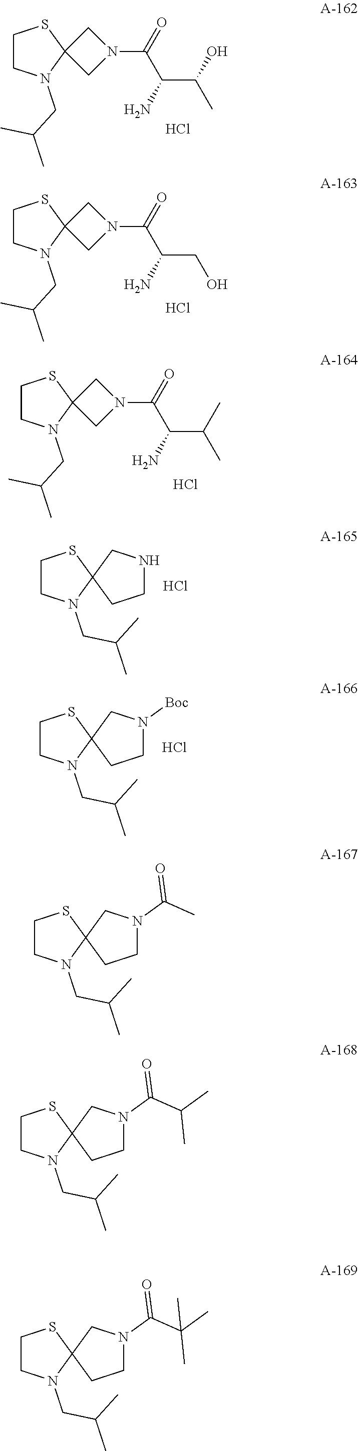

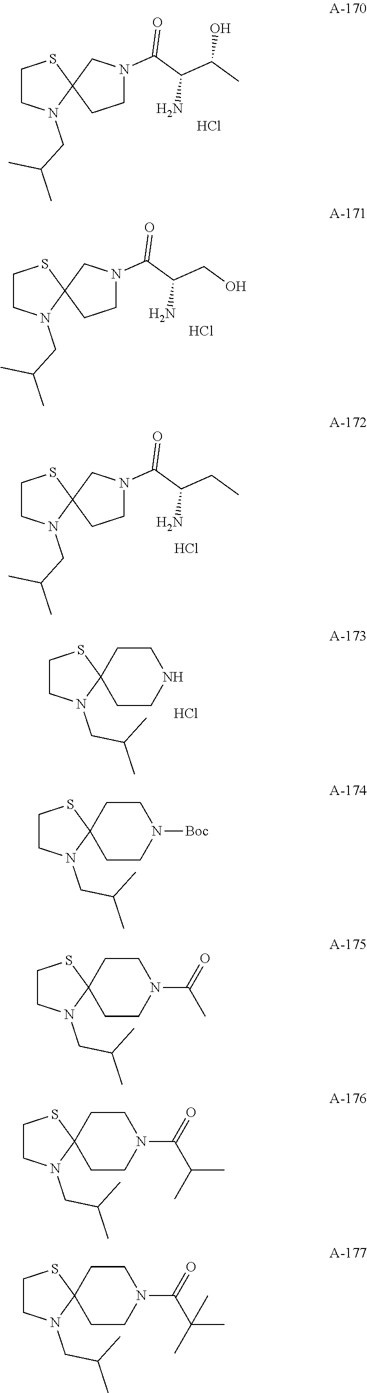

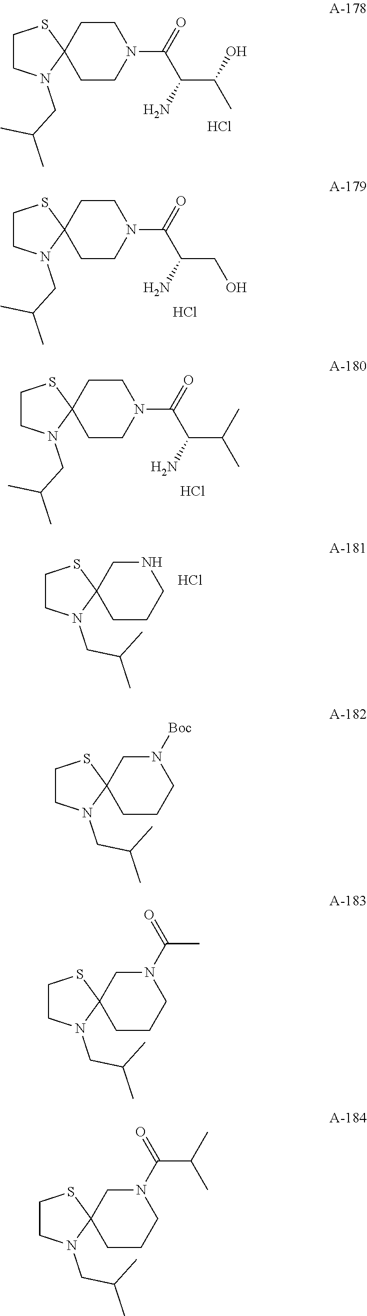

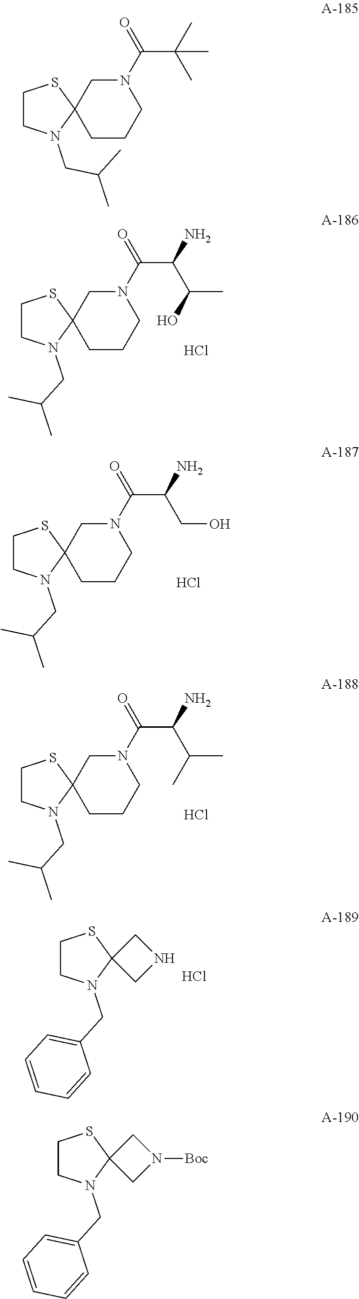

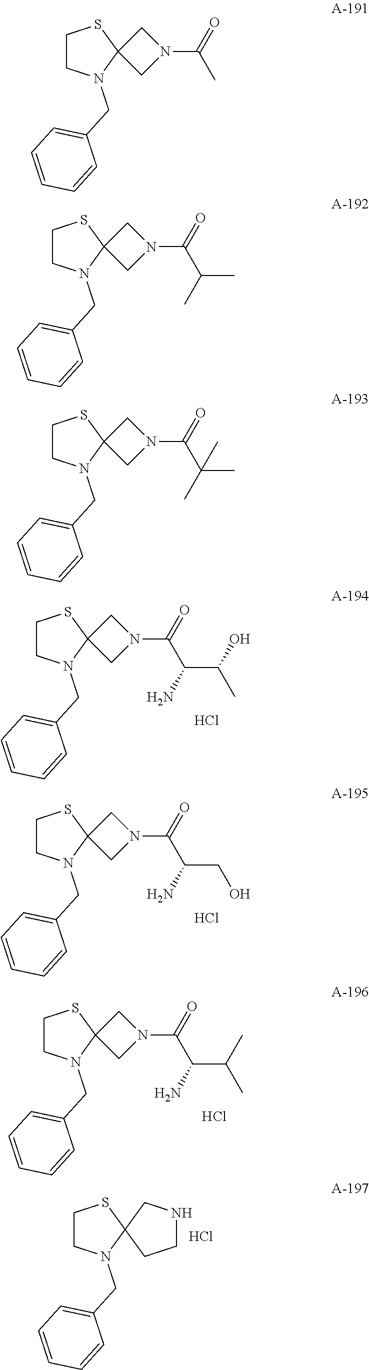

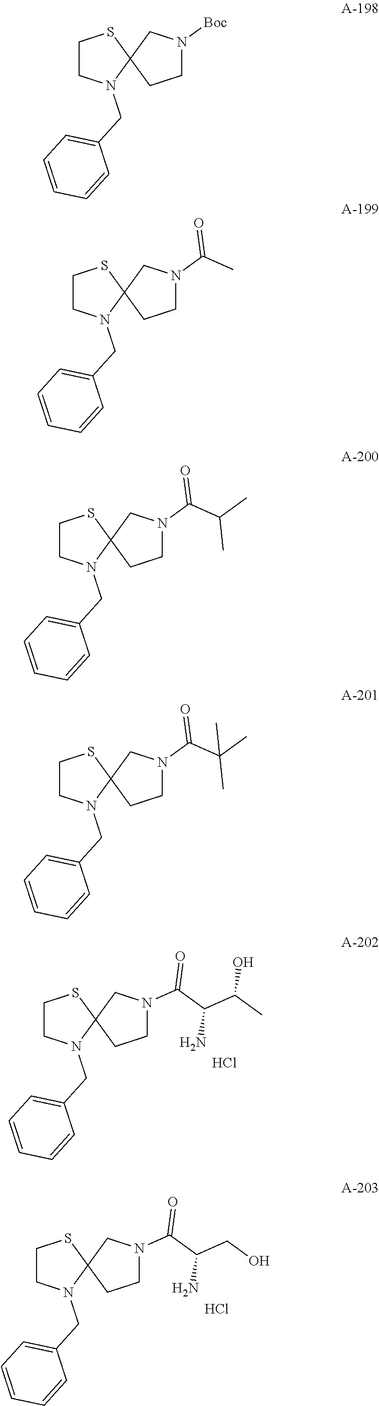

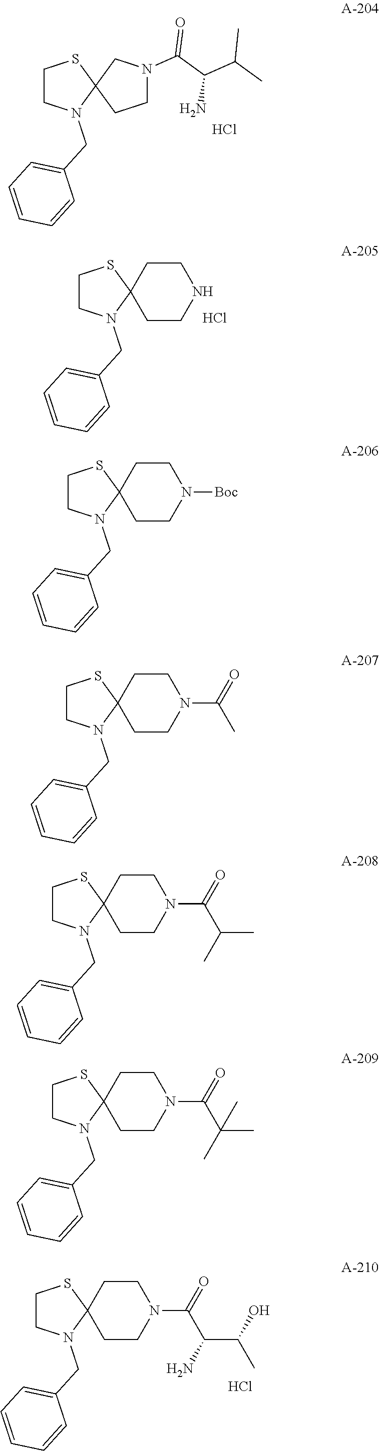

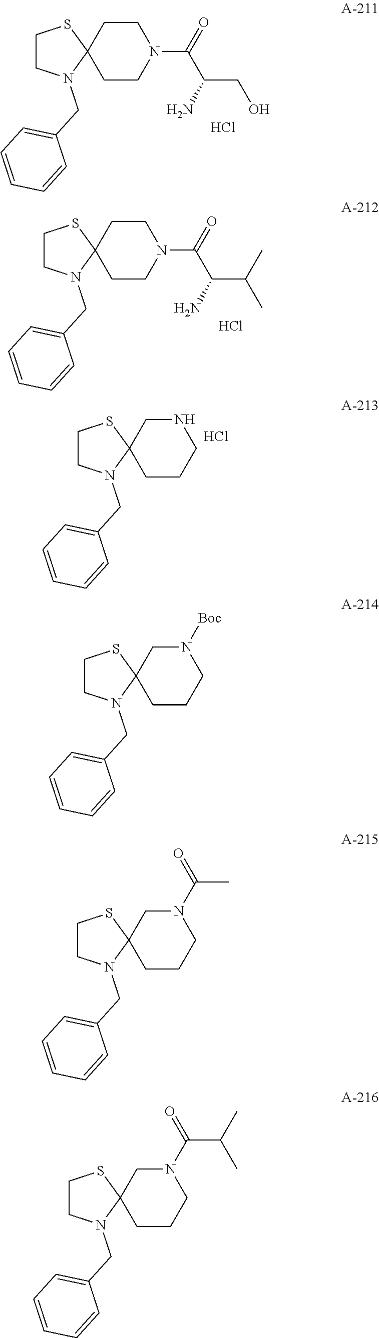

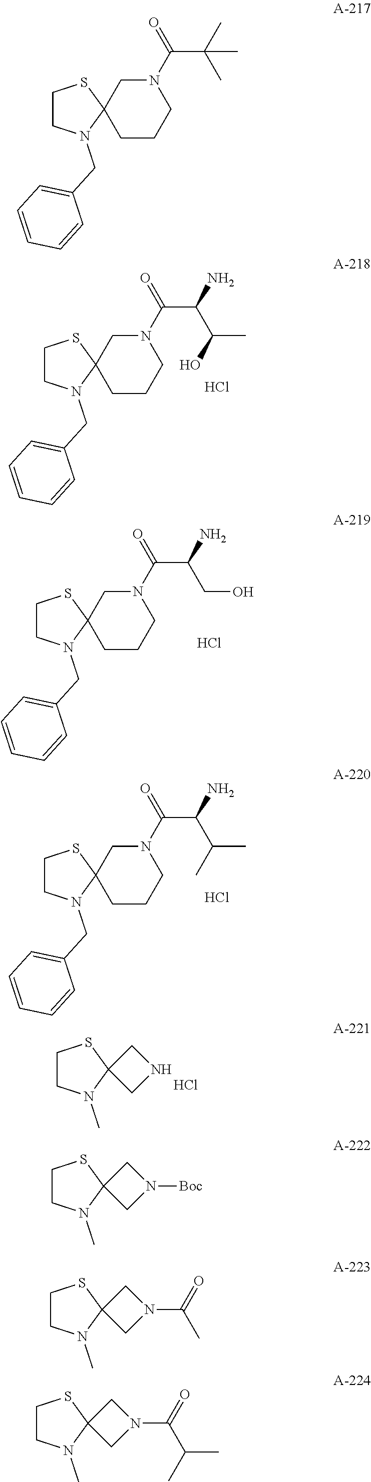

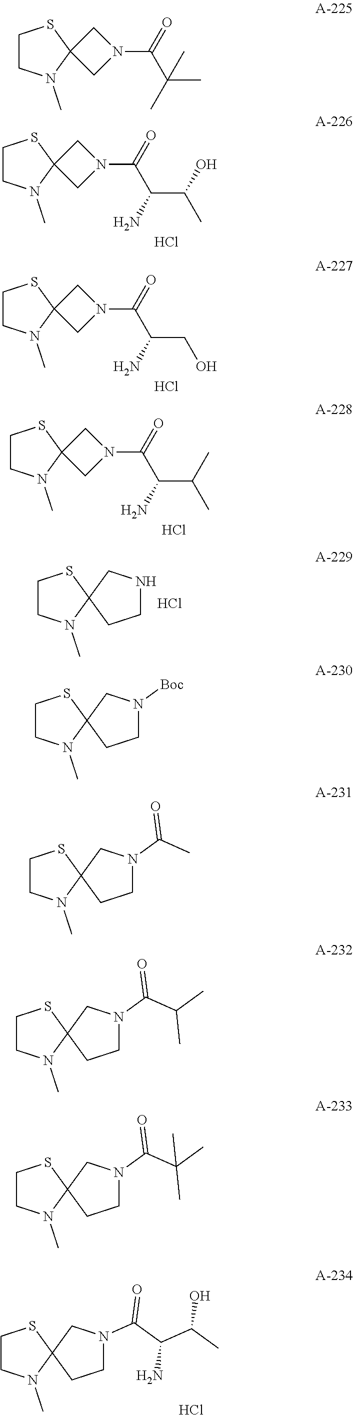

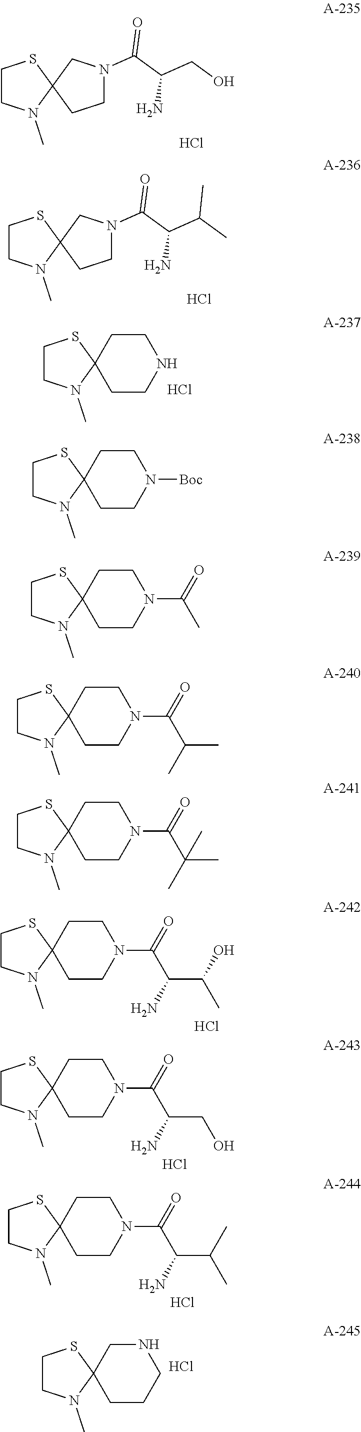

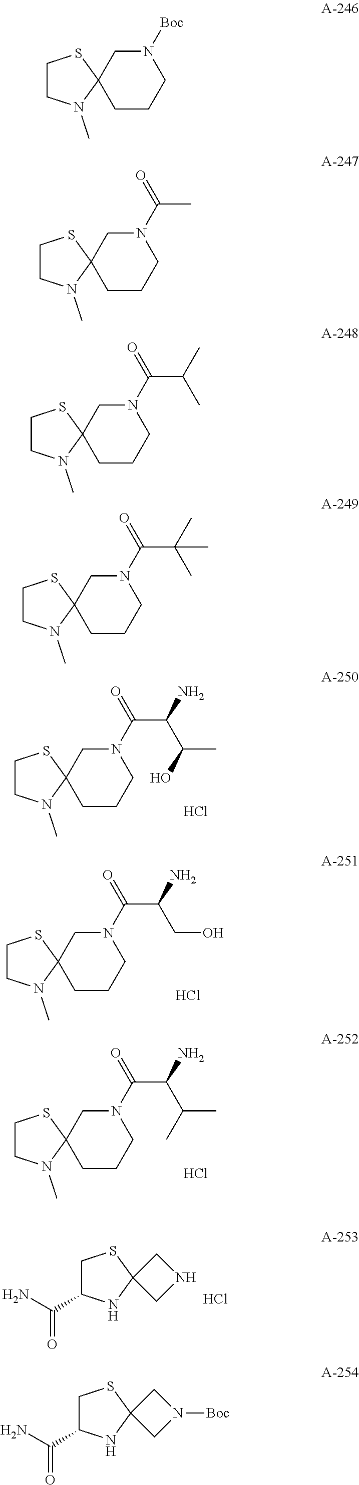

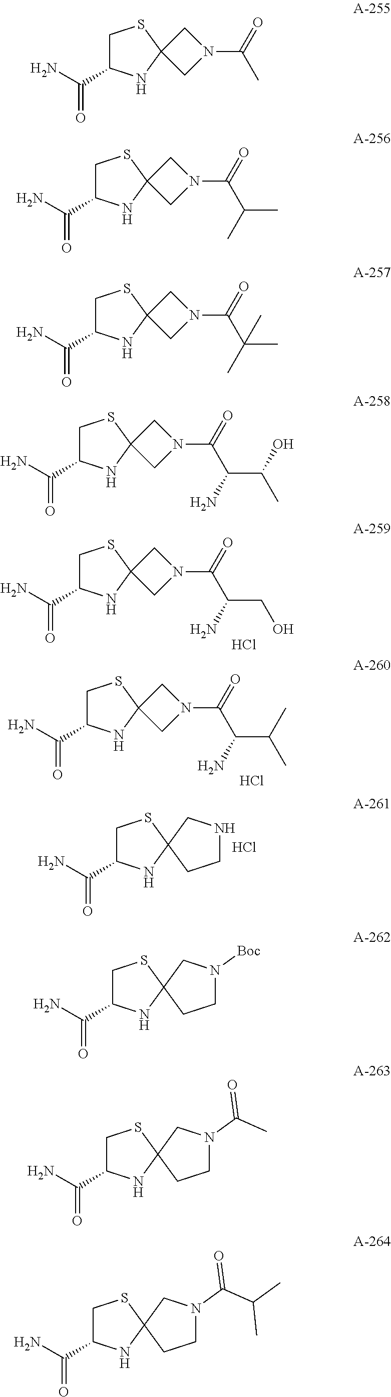

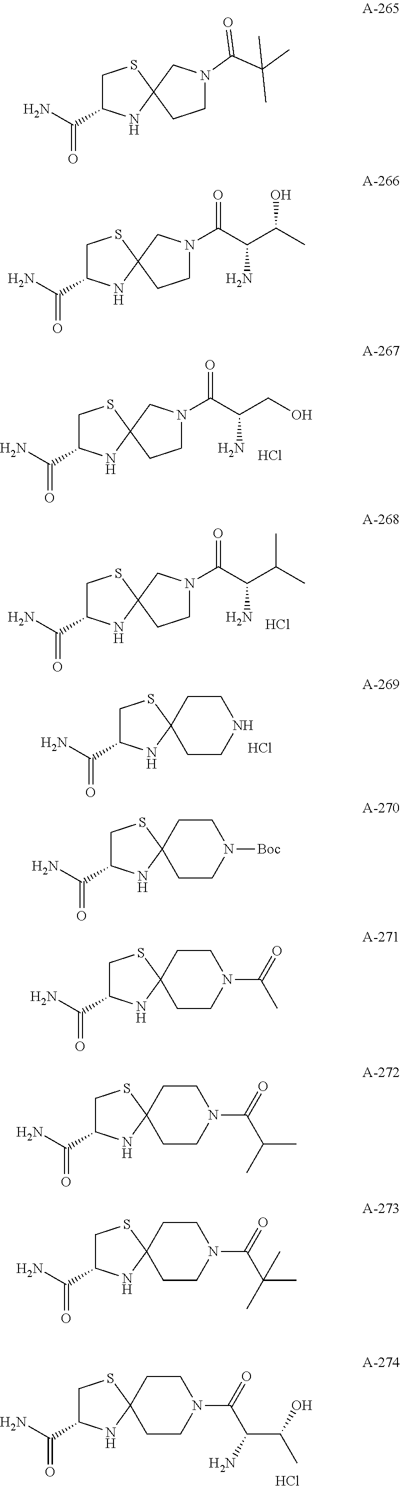

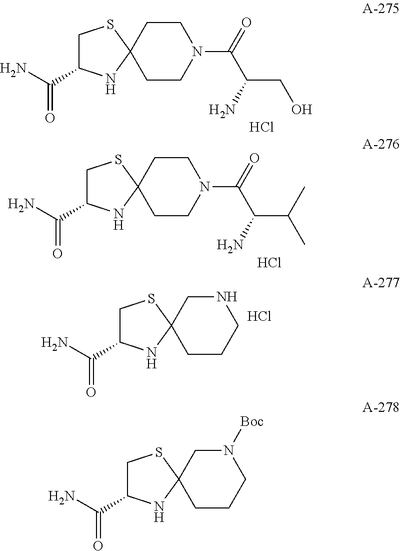

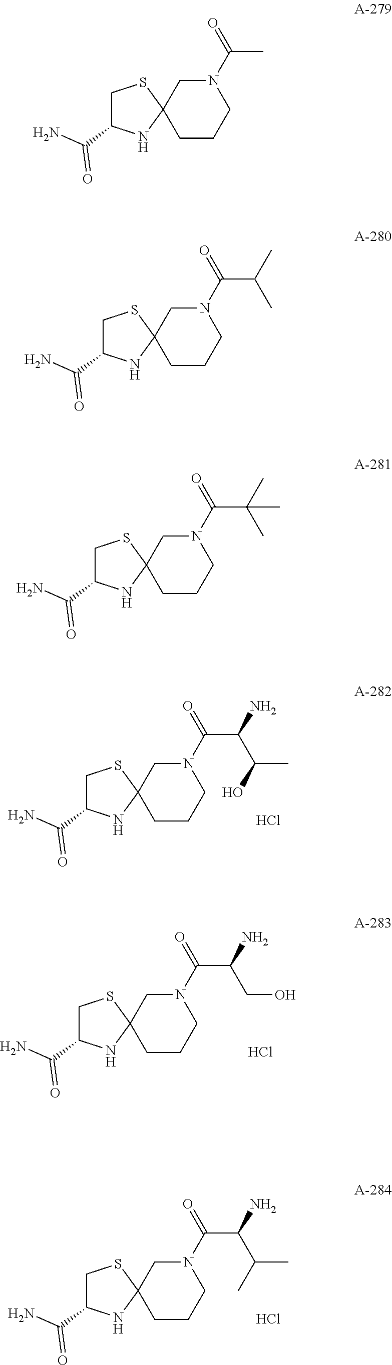

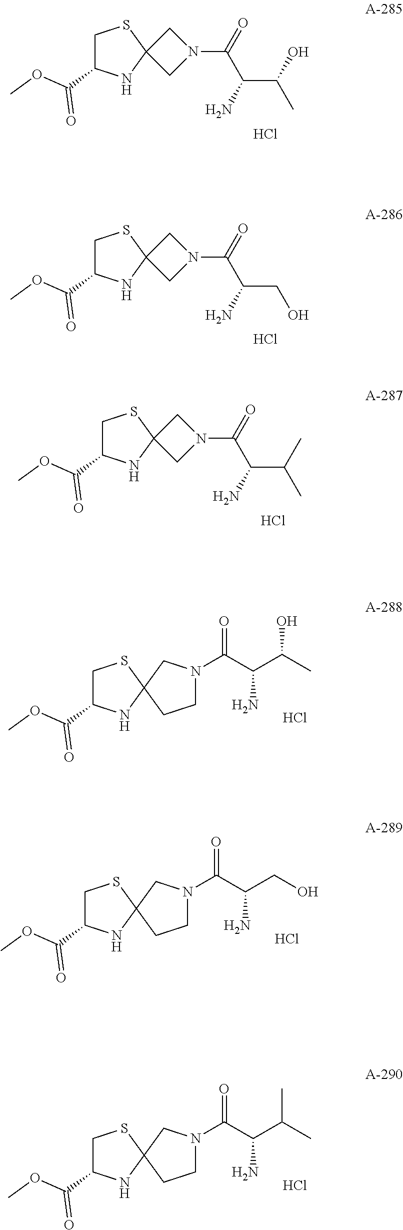

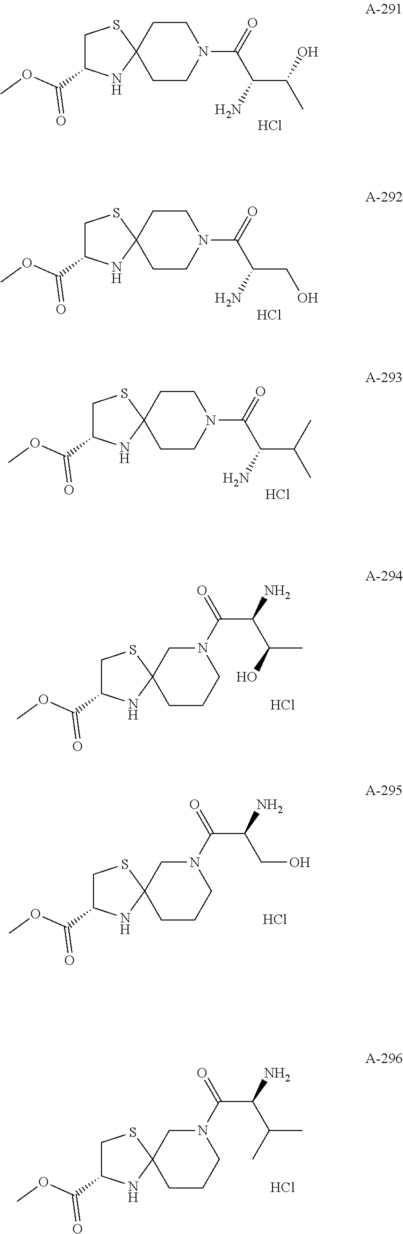

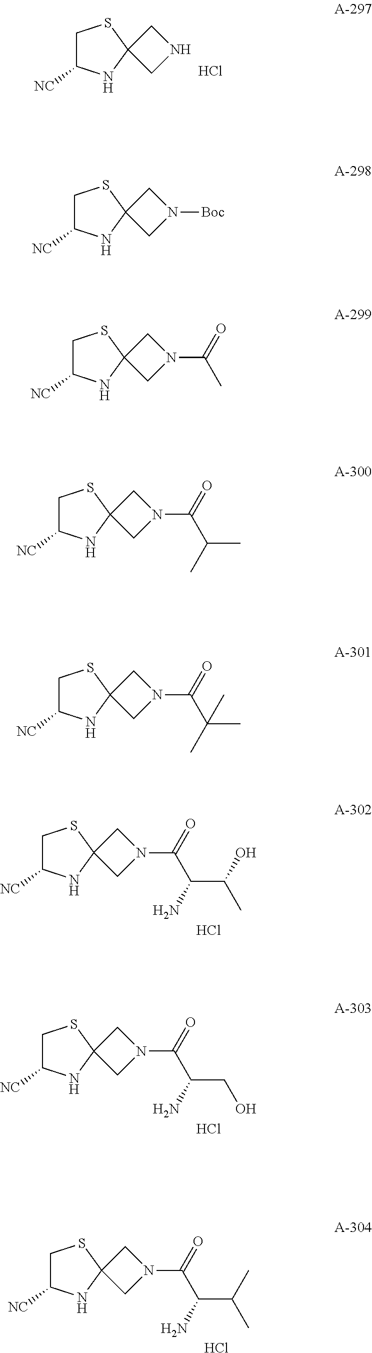

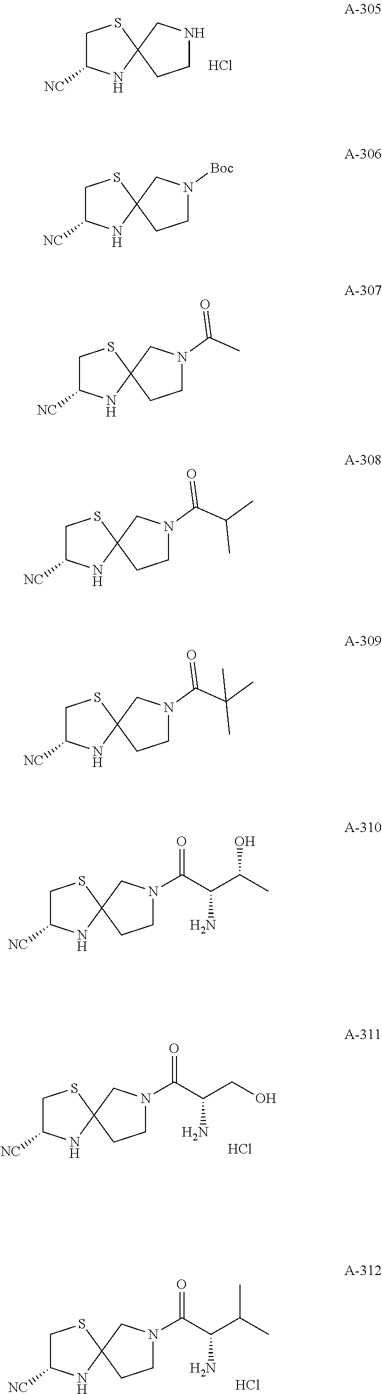

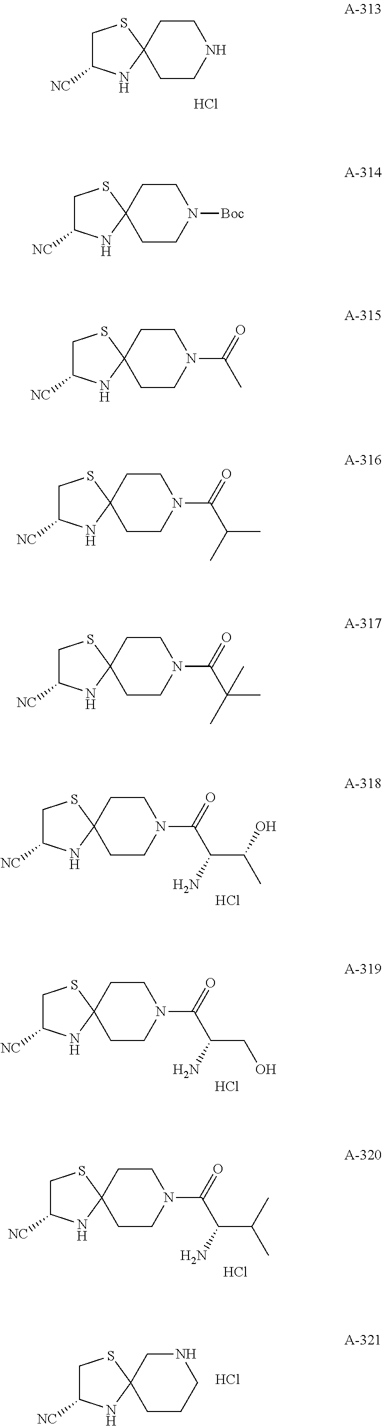

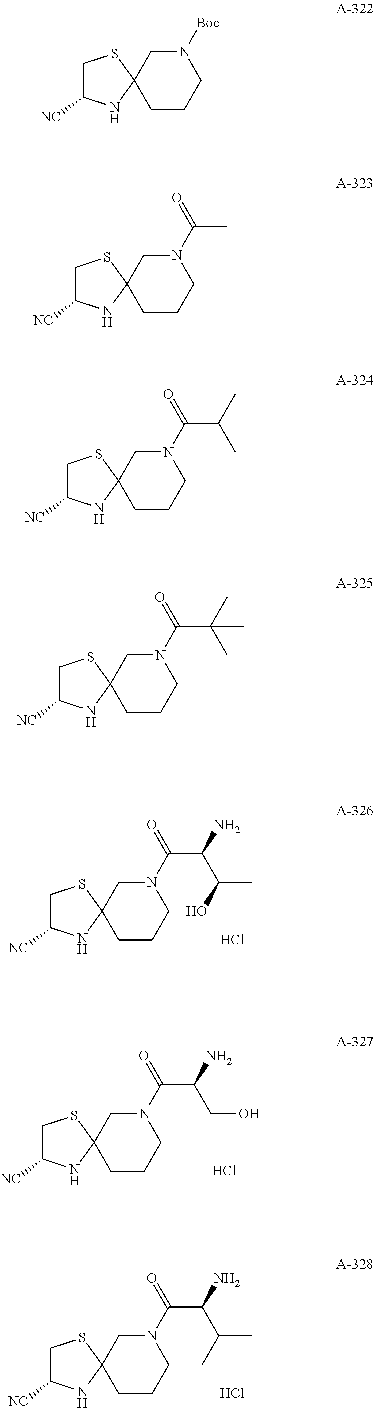

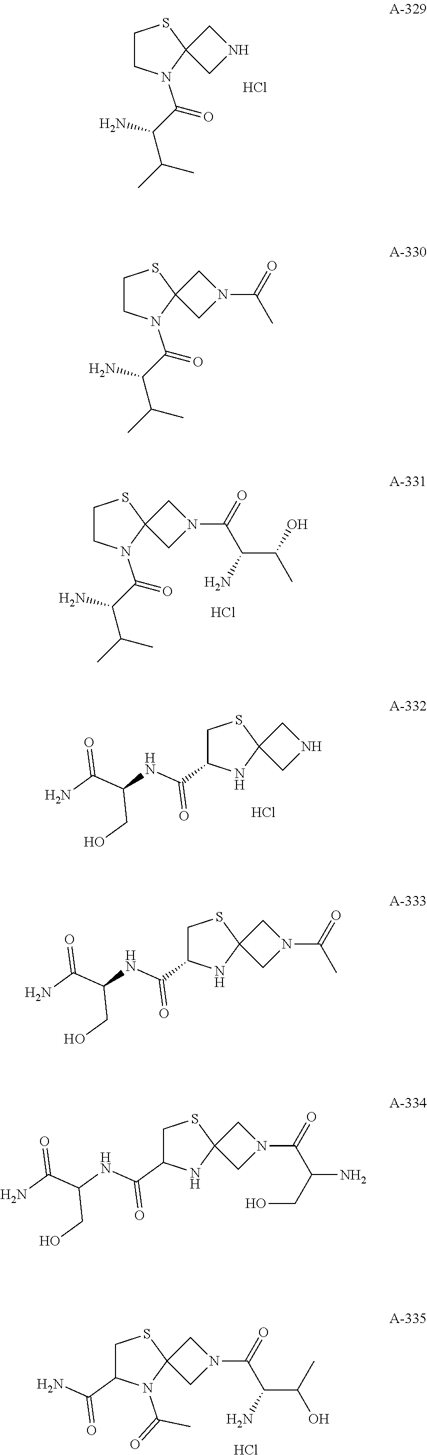

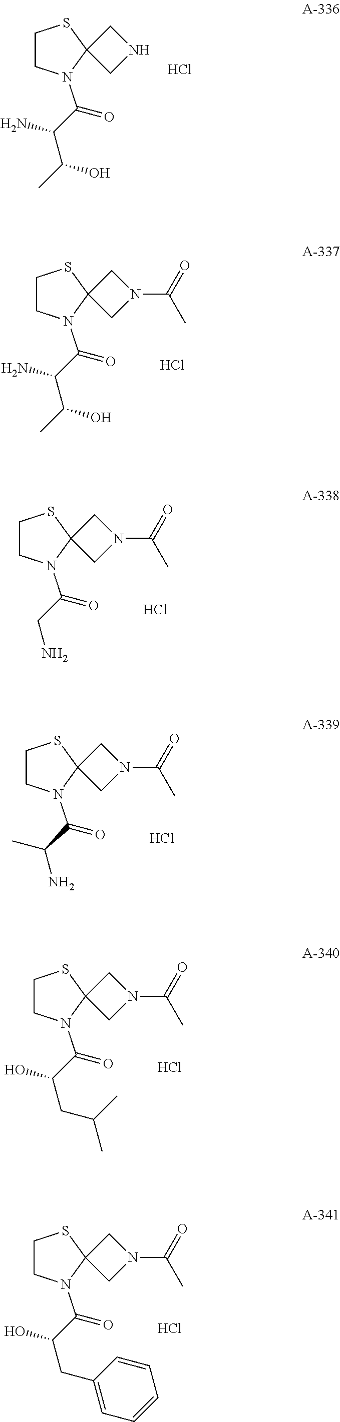

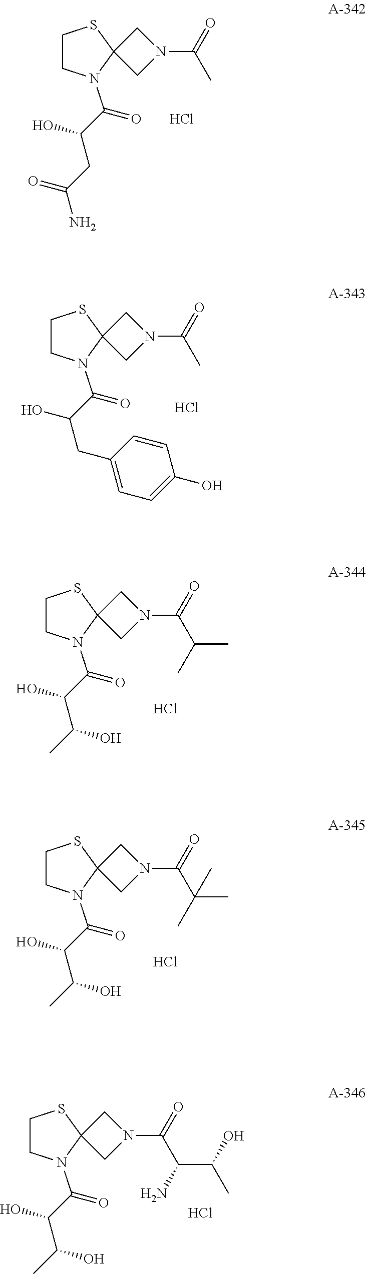

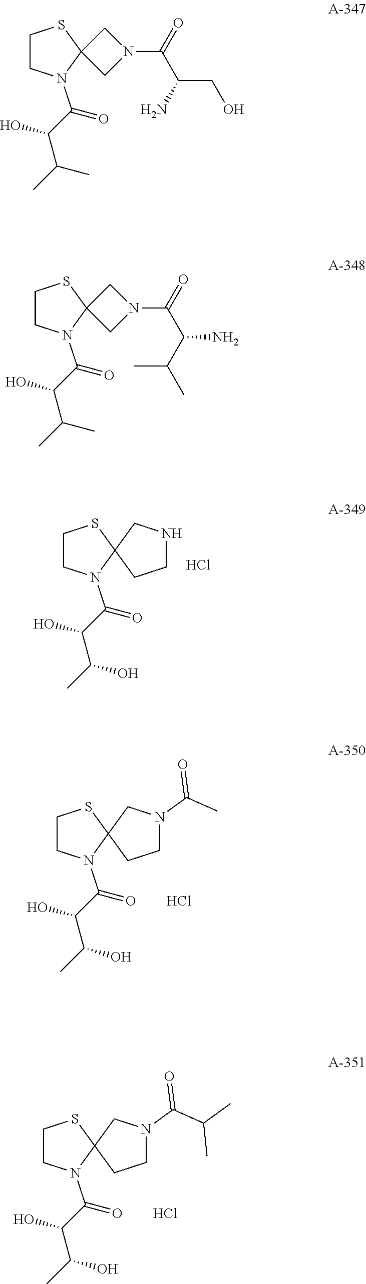

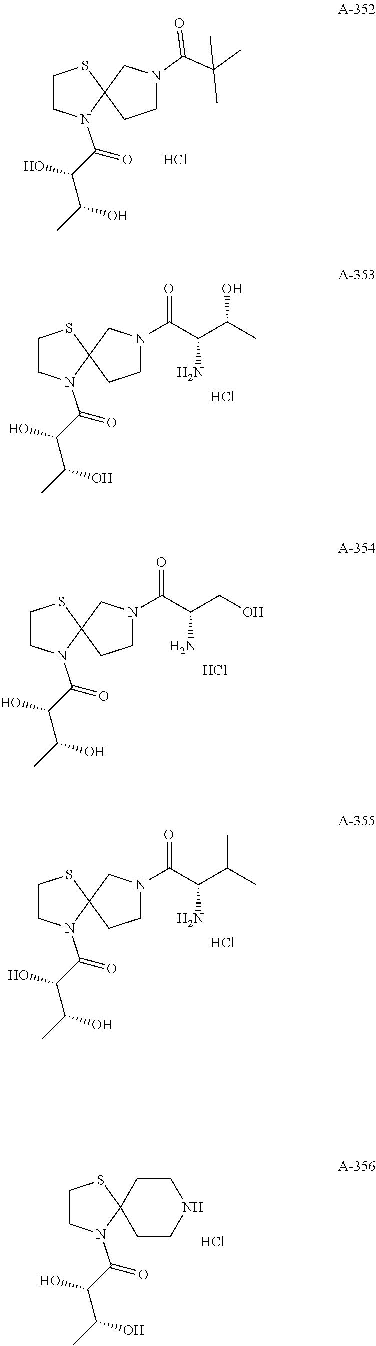

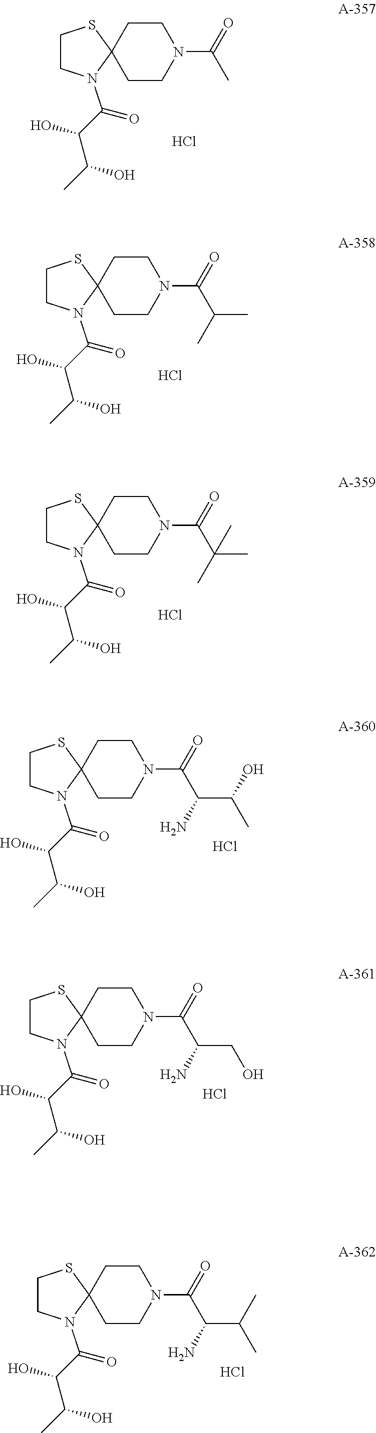

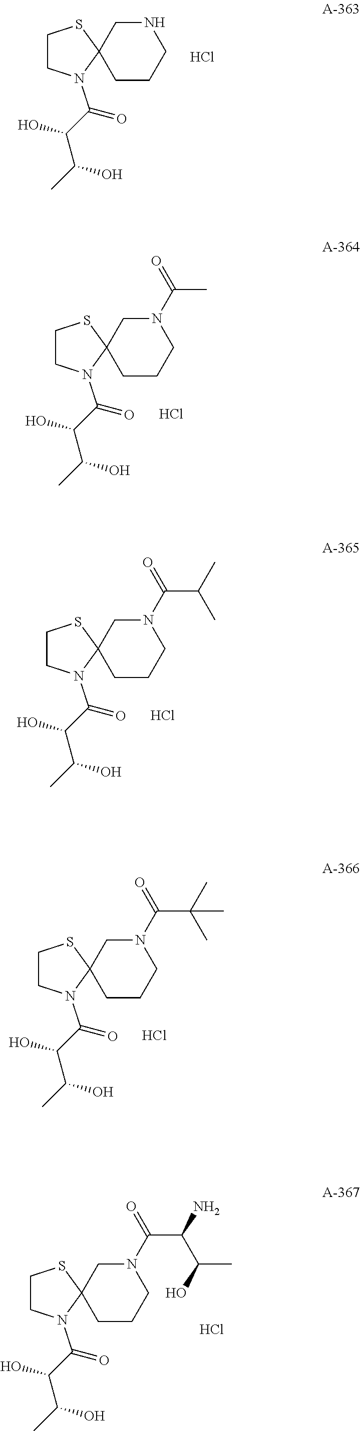

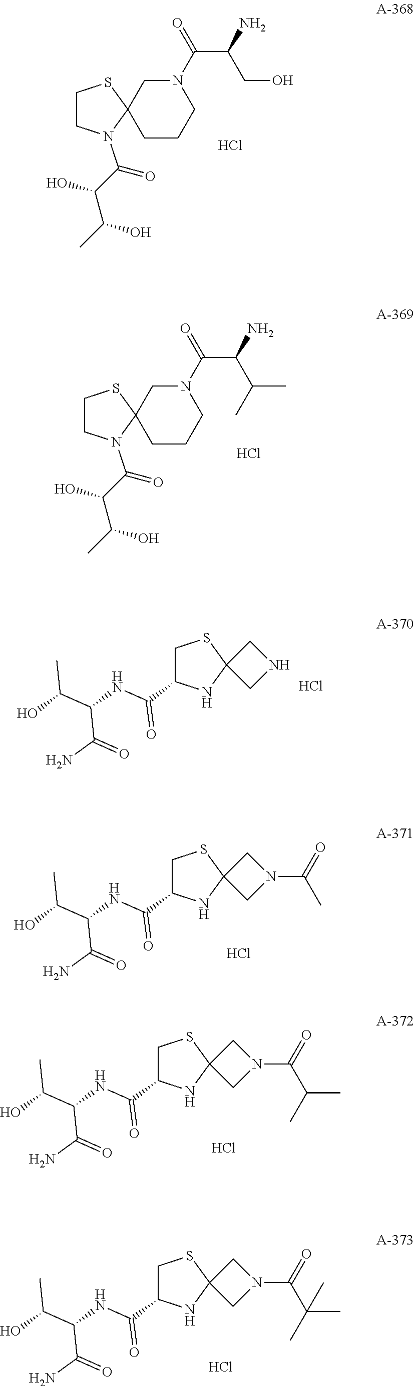

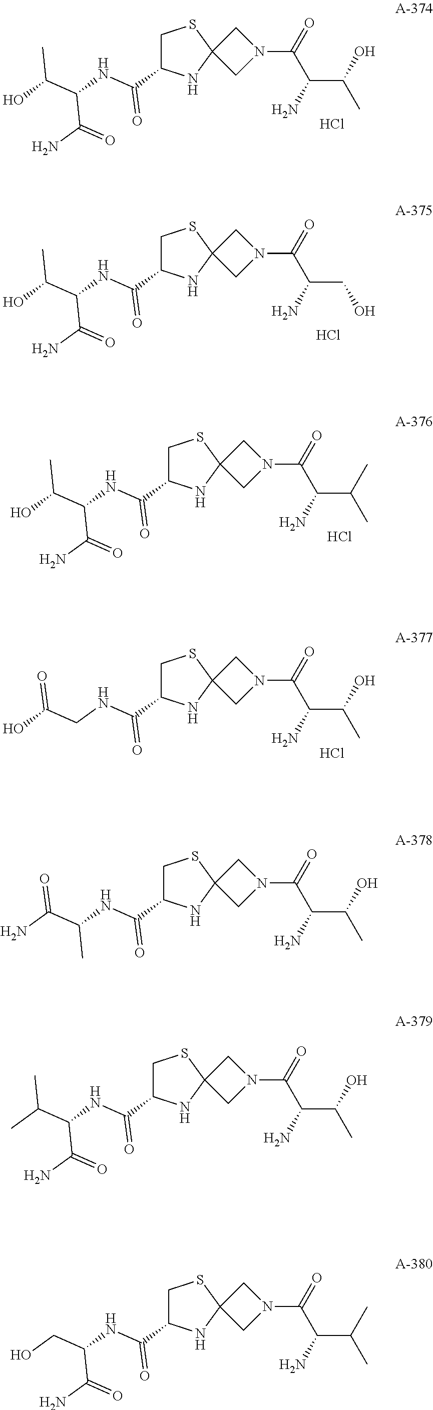

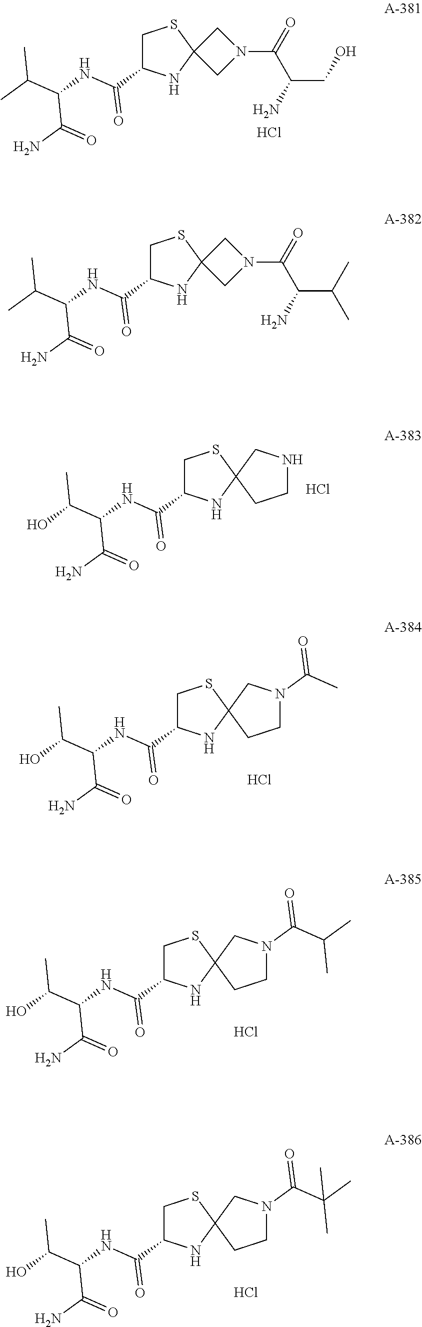

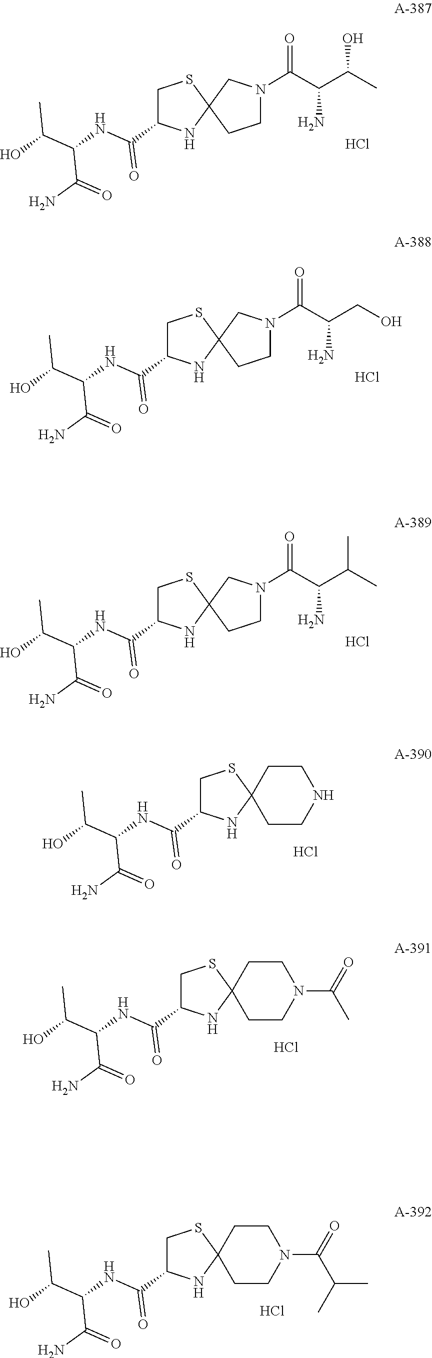

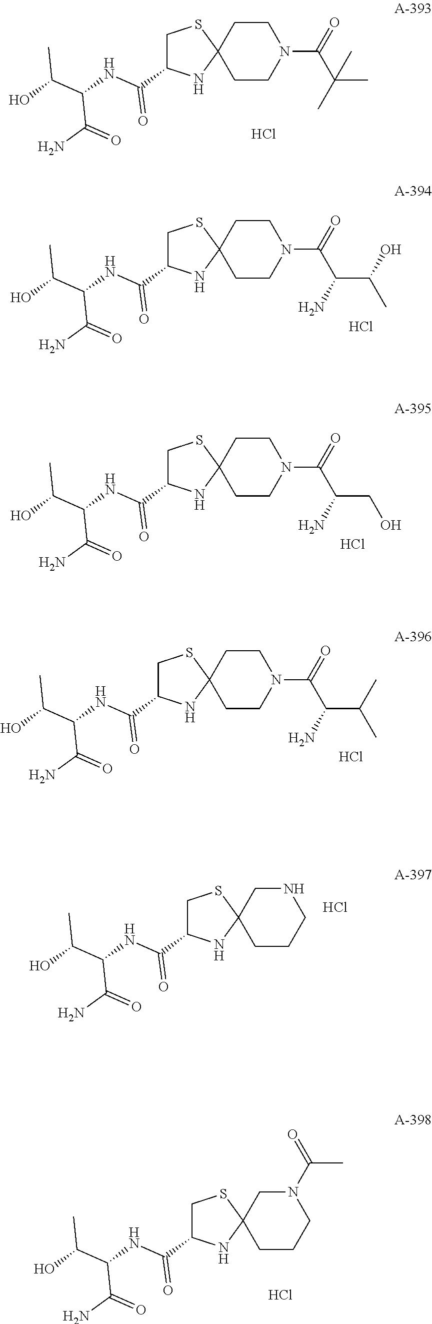

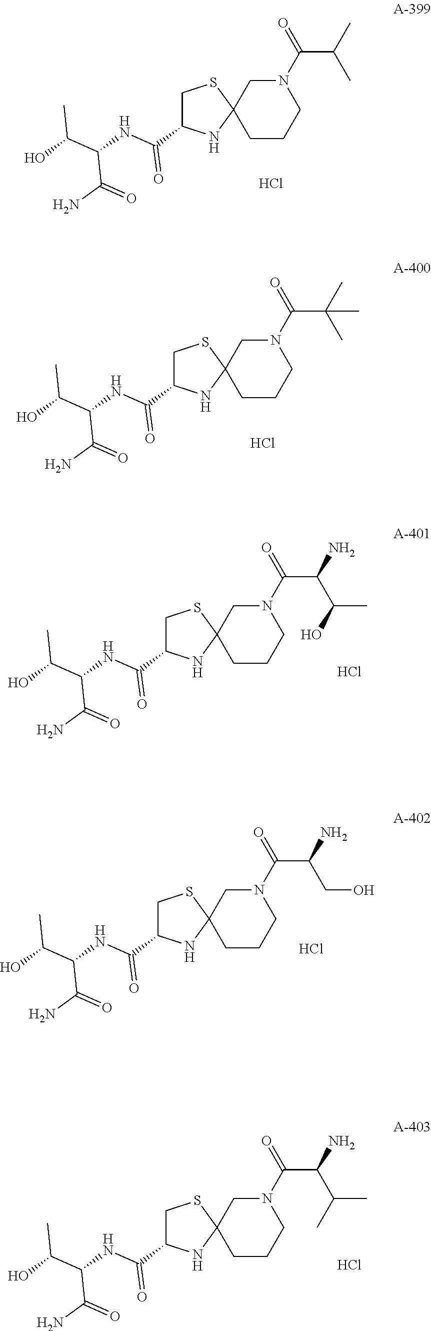

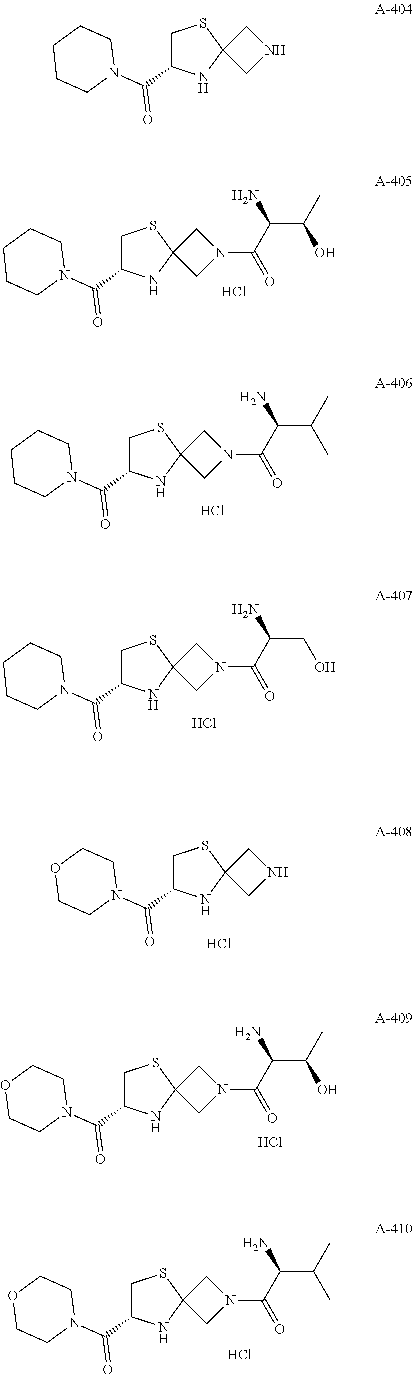

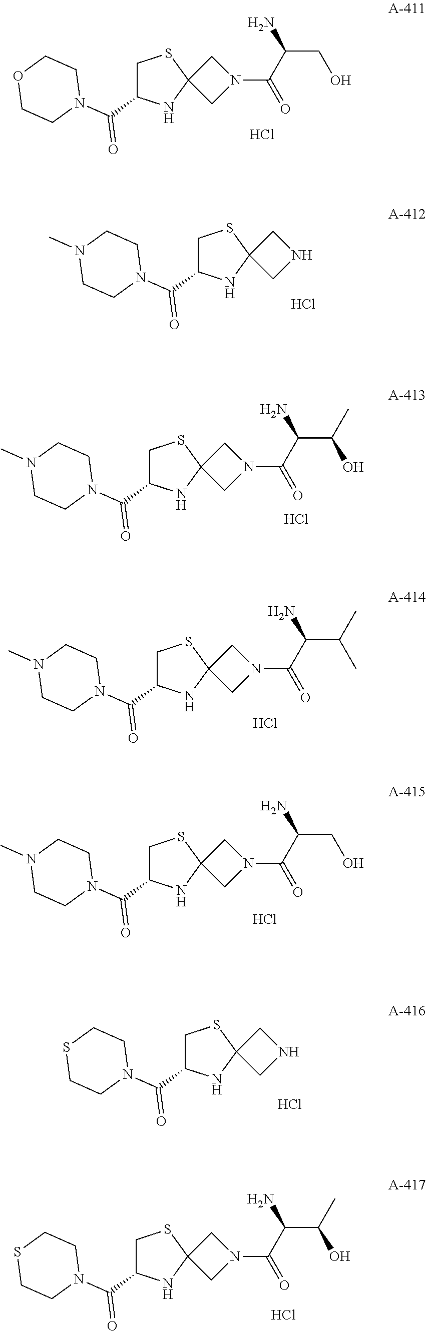

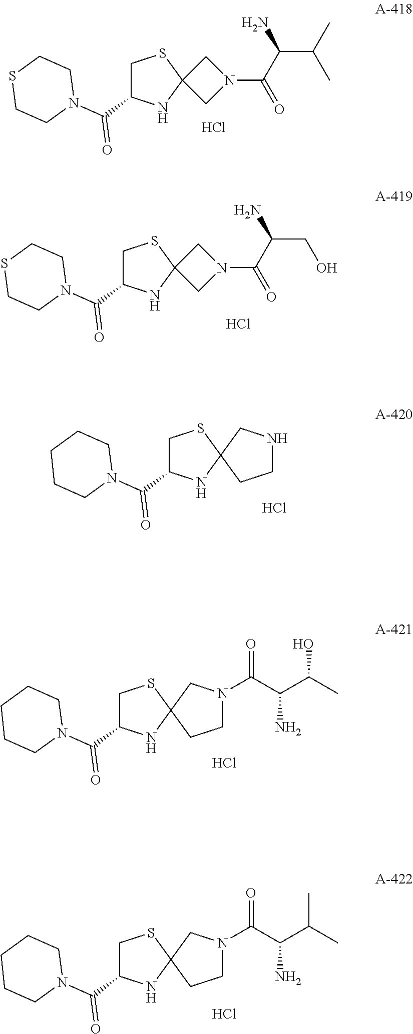

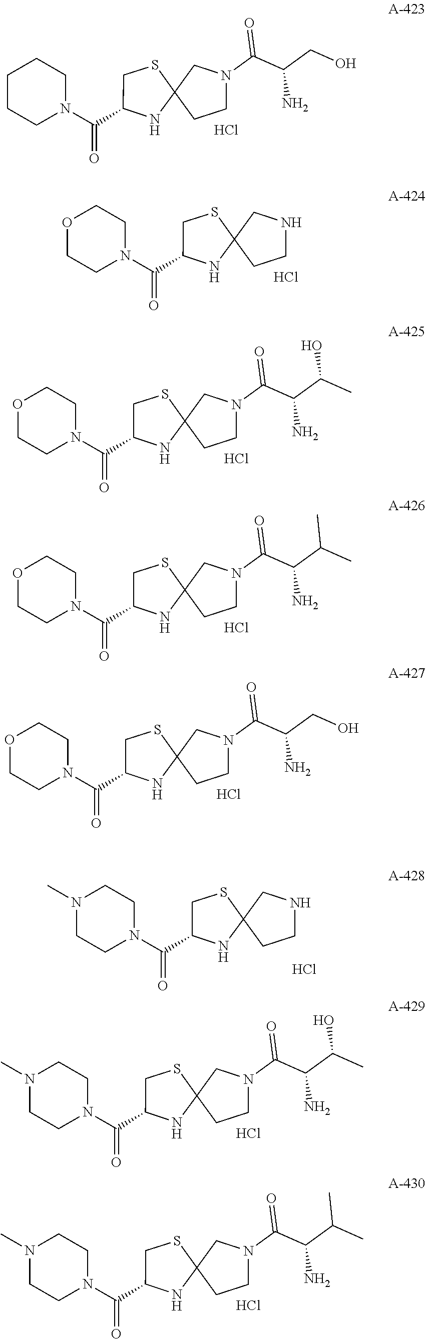

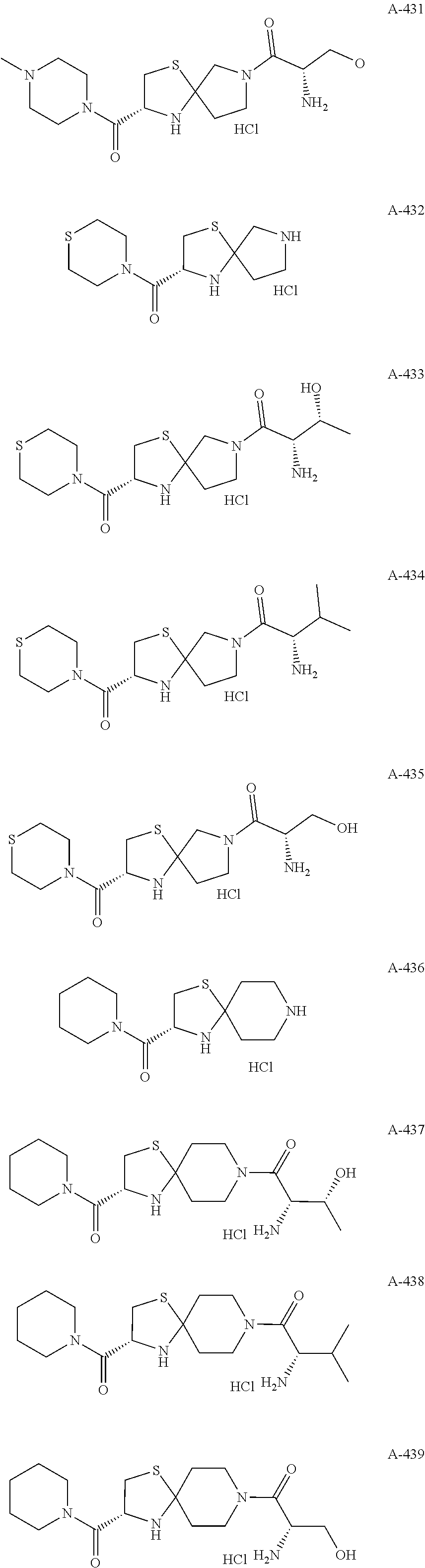

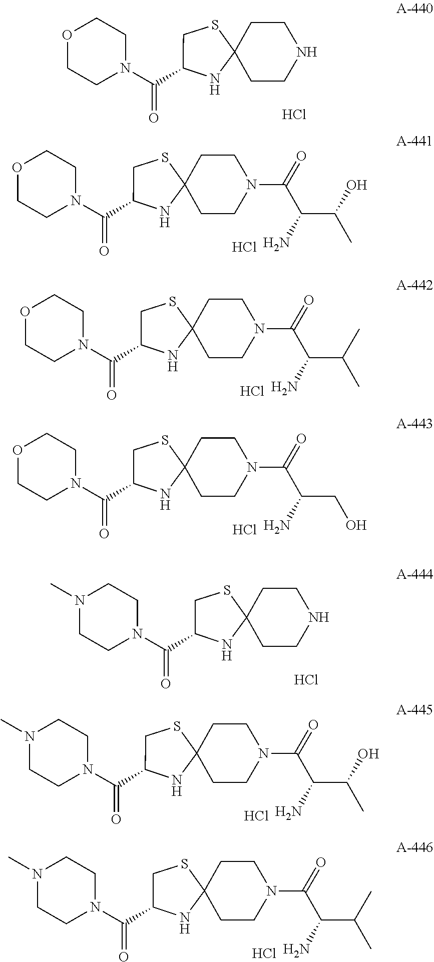

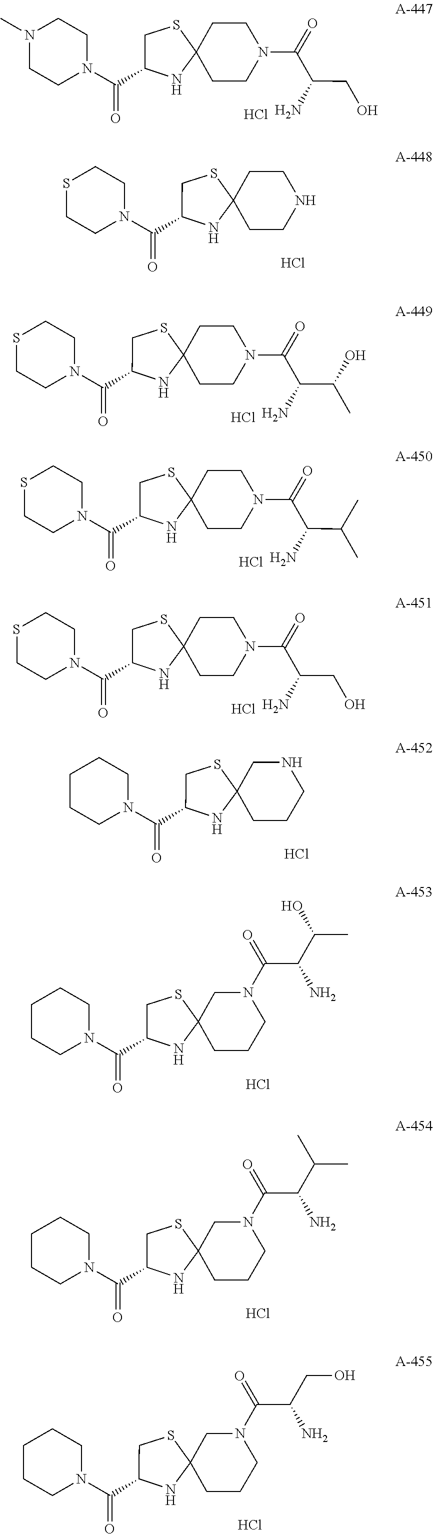

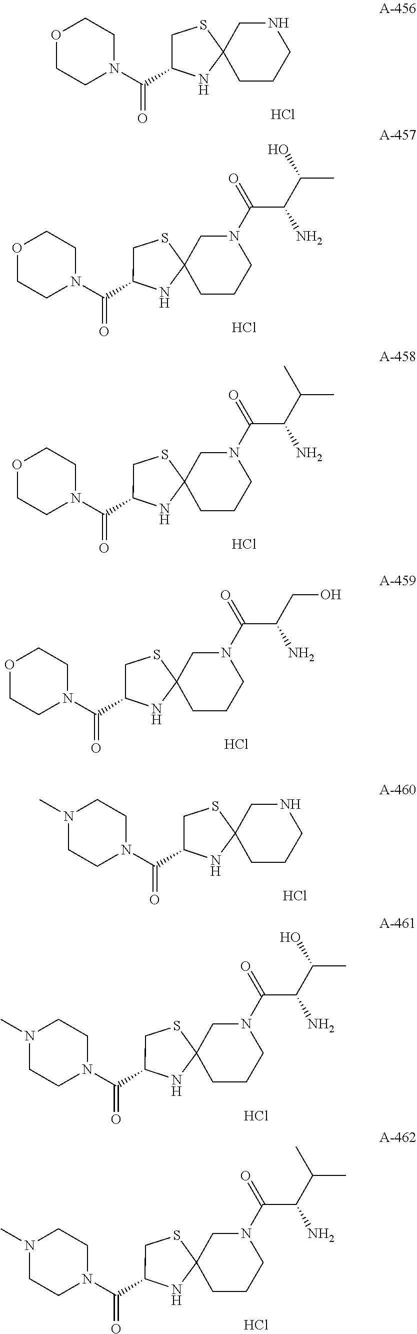

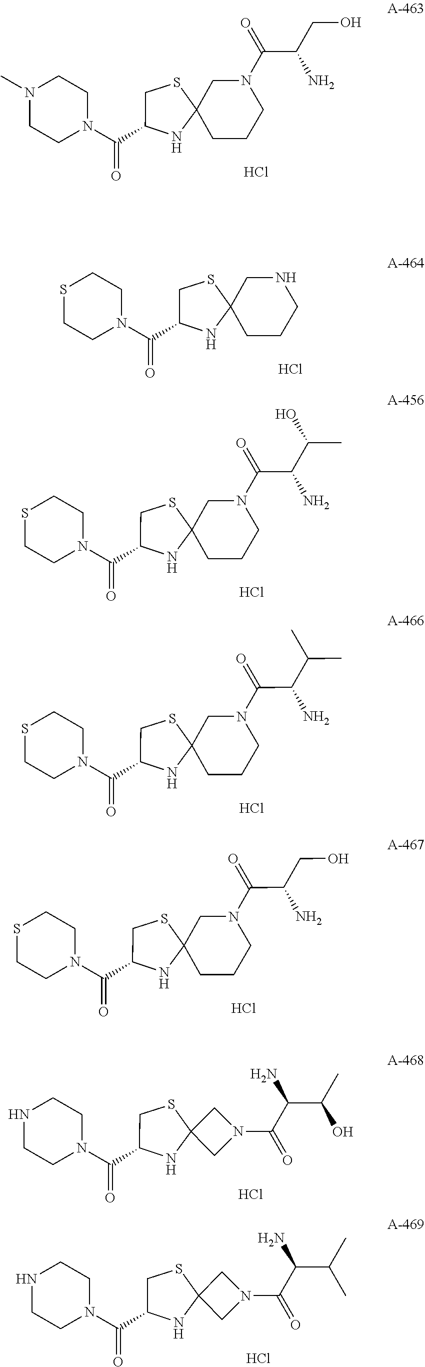

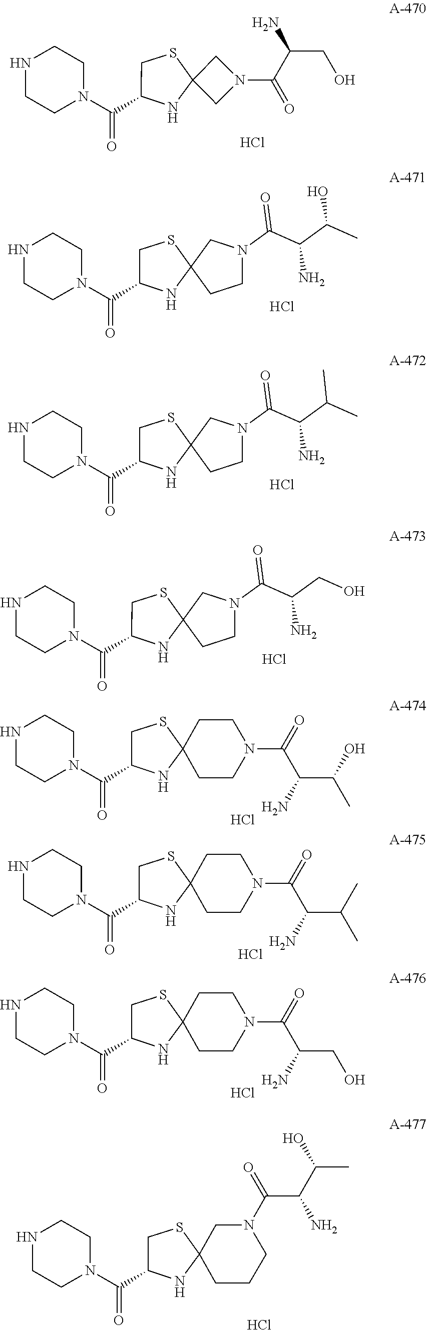

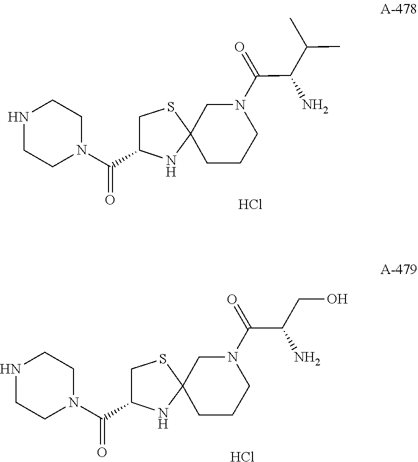 [SMILES: CC(C)[C@H](N)C(=O)N1CCCC2(C1)N[C@H](C(=O)N1CCNCC1)CS2 ; Cl ; N[C@@H](CO)C(=O)N1CCCC2(C1)N[C@H](C(=O)N1CCNCC1)CS2 ; Cl]

### Formula 202

In an embodiment, the NMDA receptor antagonist may be selected from one of the following compounds according to Formula 202. Such compounds are described in International Publication No. WO 2021/080880, published Apr. 29, 2021 and corresponding to PCT/US2020/056171, filed Oct. 16, 2020, which is incorporated by reference in its entirety herein. In the case of any conflict of terminology of Formula 202, these references incorporated by reference herein control.

In an embodiment, the NMDA receptor antagonist is a compound according to Formula 202:

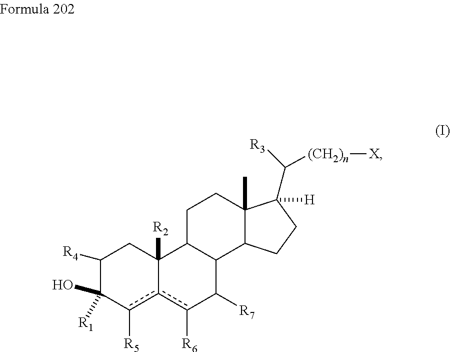

or a pharmaceutically acceptable salt thereof, wherein R1 is hydrogen, C1 to C6 alkyl, C1 to C6 alkenyl, C1 to C6 alkynyl, C1 to C6 alkoxy, or C1 to C6 haloalkyl; R2 is hydrogen, C1 to C6 alkyl, C1 to C6 alkenyl, C1 to C6 alkynyl, C1 to C6 alkoxy, C1 to C6 haloalkyl, CH₂O-RA, or —CH₂O(C═O)-RB; R3 is hydrogen, C1 to C6 alkyl, C1 to C6 alkenyl, C1 to C6 alkynyl, C1 to C6 haloalkyl, —CH₂O-RA, or —CH₂O(C═O)—RB; each RA is independently C1 to C6 alkyl or C1 to C6 haloalkyl; each RB is independently hydrogen, C1 to C6 alkyl, or C1 to C6 haloalkyl; R4 is hydrogen, hydroxy, C1 to C6 alkyl, C1 to C6 alkenyl, C1 to C6 alkynyl, C1 to C6 alkoxy, or C1 to C6 haloalkyl; R5 is hydrogen, hydroxy, C1 to C6 alkyl, C1 to C6 alkenyl, C1 to C6 alkynyl, C1 to C6 alkoxy, or C1 to C6 haloalkyl; R6 is hydrogen, hydroxy, C1 to C6 alkyl, C1 to C6 alkenyl, C1 to C6 alkynyl, C1 to C6 alkoxy, or C1 to C6 haloalkyl; R7 is hydrogen, hydroxy, C1 to C6 alkyl, C1 to C6 alkenyl, C1 to C6 alkynyl, C1 to C6 alkoxy, or C1 to C6 haloalkyl; X is hydroxy, OSO3H, OSO3-NH4+, SO₃H, SO₂H, COOH, NH(CH₂)ₘ—Y, H2PO4, HPO3R8, C(R9)2COOH, C(R9)2-Y, or other negatively charged group at physiological pH; Y is OSO3H, SO₃H, SO₂H, COOH, H2PO4, HPO3R8, or other negatively charged group at physiological pH; each R8 is independently hydrogen, C1 to C6 alkyl or C1 to C6 haloalkyl; each R9 is independently hydrogen, C1 to C6 alkyl, C1 to C6 haloalkyl, or halo; m is an integer from 1 to 6; and n is an integer from 1 to 8.

In further embodiments, the compound according to Formula 202 is:

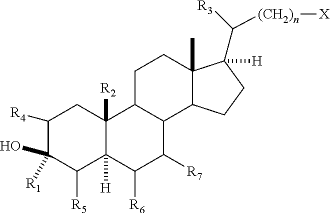

or a pharmaceutically acceptable salt thereof, where R1, R2, R3, R4, R5, R6, R7, and X are as defined above.

In further embodiments, the compound according to Formula 202 is:

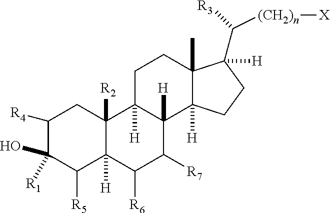

or a pharmaceutically acceptable salt thereof, where R1, R2, R3, R4, R5, R6, R7, and X are as defined above.

In further embodiments, the compound according to Formula 202 is selected from:

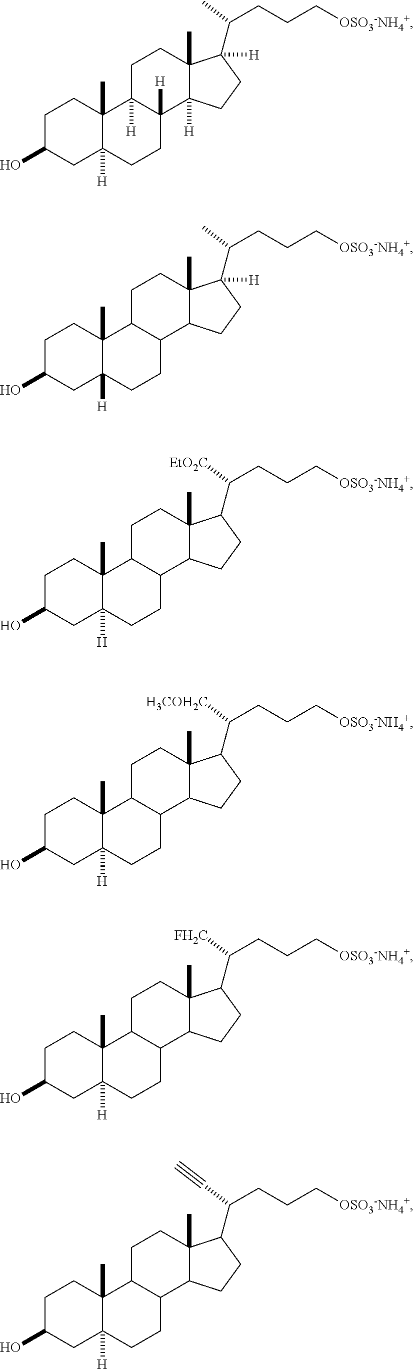

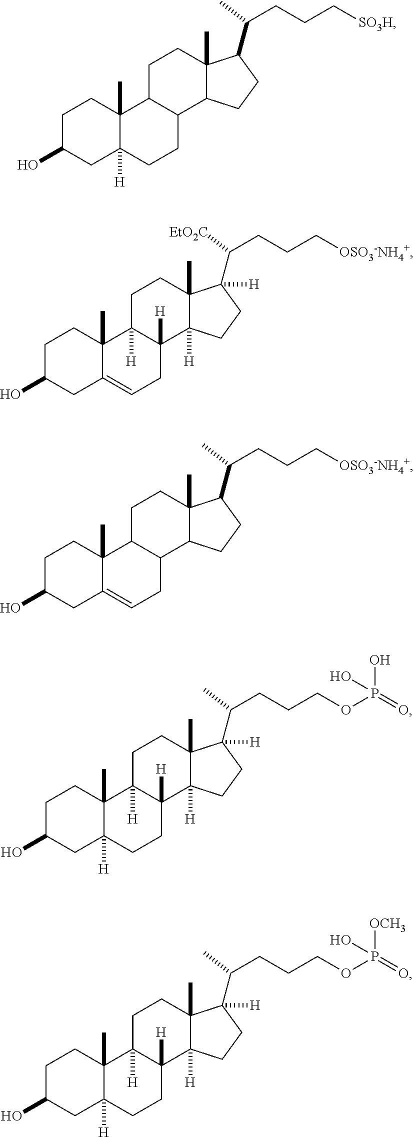

or pharmaceutically acceptable salts thereof.

### Formula 203

In an embodiment, the NMDA receptor antagonist may be selected from one of the following compounds according to Formula 203. Such compounds are described in WO2020/153414, published Jul. 30, 2020 and corresponding to PCT/JP2020/002216, filed Jan. 23, 2020, and US20220089525, published Mar. 24, 2022, which are each incorporated by reference in their entirety herein. In the case of any conflict of terminology of Formula 203, these references incorporated by reference herein control.

In some embodiments, the compound according to Formula 203 is:

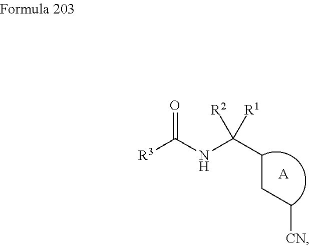

and/or wherein ring A is a 6-membered aromatic ring further substituted by 1 to 4 substituents selected from (1) a C1-3 alkyl group, (2) a C1-3 haloalkyl group, (3) an optionally substituted cyclic group, (4) an optionally substituted C1-6 alkoxy group, and (5) a halogen atom;
R1 and R2 are each independently a hydrogen atom or a C1-3 alkyl group optionally substituted by fluorine atom(s);
R3 is (1) a group represented by

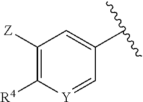

wherein R4 is a substituent selected from (1) a C1-3 haloalkyl group, (2) an optionally substituted C3-7 cycloalkyl group, and (3) an optionally substituted C1-6 alkoxy group, Y is a nitrogen atom or CR6, R6 is a hydrogen atom or a halogen atom, and Z is a hydrogen atom or a halogen atom, or (2) a group represented by

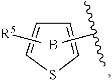

wherein R5 is a substituent selected from
(1) a C1-3 haloalkyl group, (2) an optionally substituted C3-7 cycloalkyl group, and (3) an optionally substituted C1-6 alkoxy group, and ring B is a thiophene ring optionally further substituted by one halogen atom, or a salt thereof.

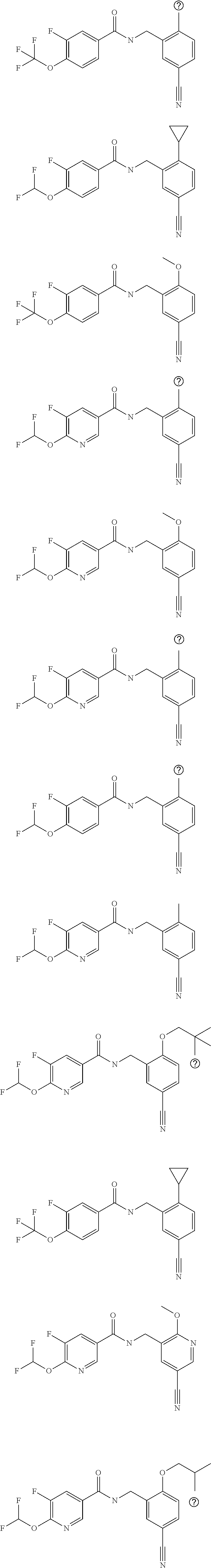

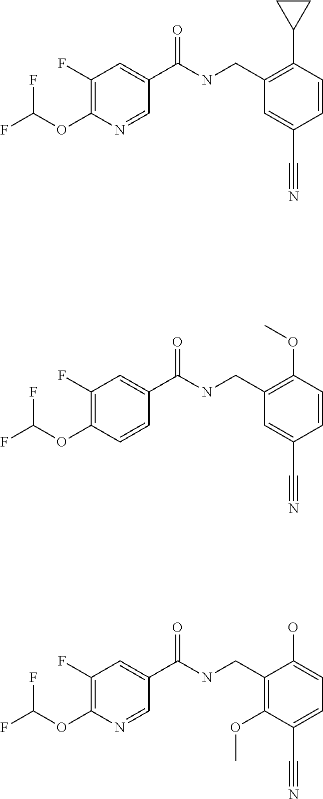 [SMILES: N#Cc1ccc(C2CC2)c(CNC(=O)c2cnc(OC(F)F)c(F)c2)c1 ; COc1ccc(C#N)cc1CNC(=O)c1ccc(OC(F)F)c(F)c1 ; COc1c(C#N)ccc(O)c1CNC(=O)c1cnc(OC(F)F)c(F)c1]

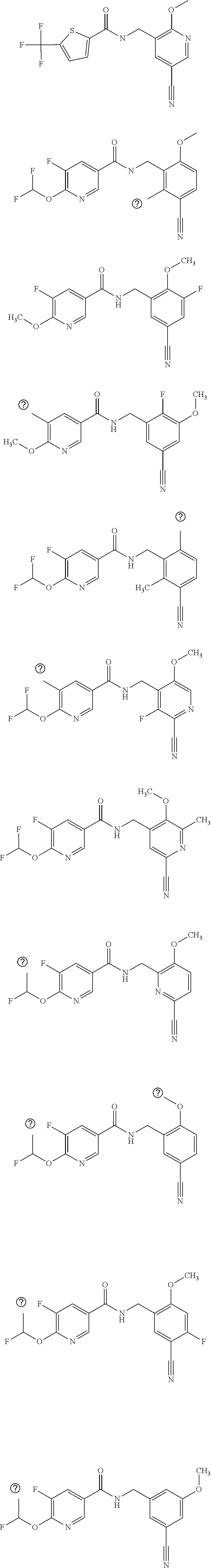

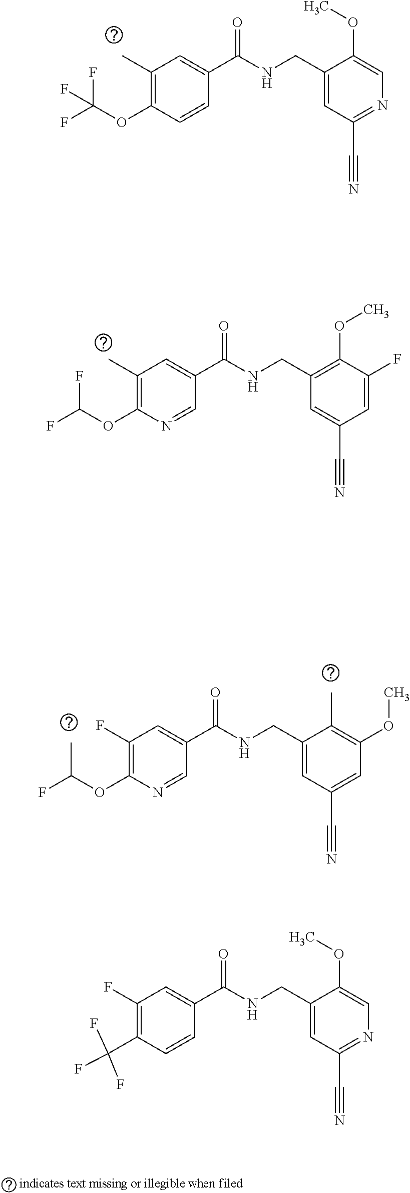 [SMILES: COc1cnc(C#N)cc1CNC(=O)c1ccc(OC(F)(F)F)c(C)c1 ; COc1c(F)cc(C#N)cc1CNC(=O)c1cnc(OC(F)F)c(C)c1 ; COc1cc(C#N)cc(CNC(=O)c2cnc(OC(C)F)c(F)c2)c1C ; COc1cnc(C#N)cc1CNC(=O)c1ccc(C(F)(F)F)c(F)c1]

### Formula 204

In an embodiment, the NMDA receptor antagonist may be selected from one of the following compounds according to Formula 204. Such compounds are described in WO2019/210130, published Oct. 31, 2019, corresponding to PCT/US2019/029285, filed Apr. 26, 2019, and US2021/0094920, published Apr. 1, 2021, which are each incorporated by reference in their entirety herein. In the case of any conflict of terminology of Formula 204, these references incorporated by reference herein control.

In an embodiment, the NMDA receptor antagonist is:

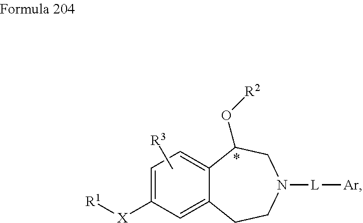

wherein
X is O or S, specifically O; R1 is H, C1-C6 alkyl, C1-C6 haloalkyl, specifically C1-C3 alkyl, and more specifically methyl; R2 is H, C1-C6 alkyl, C1-C6 haloalkyl, specifically H or C1-C3 alkyl, and more specifically H; R3 is H, C1-C6 alkyl, C1-C6 alkoxy, C1-C6 haloalkyl, C1-C6 haloalkoxy, —OH, halo, —COOH, —CN, —NO2, amino, mono- or di-alkylamino, C2-C6 alkanoyl, C2-C8 cycloalkyl, or heterocycloalkyl, specifically H, C1-C3 alkyl, or C1-C3 haloalkyl, and more specifically H; L is a linking group; and Ar is an aryl or heteroaryl group, each of which is optionally substituted with one, two, or three substituents.

In further embodiments, the NMDA receptor antagonist is selected from:

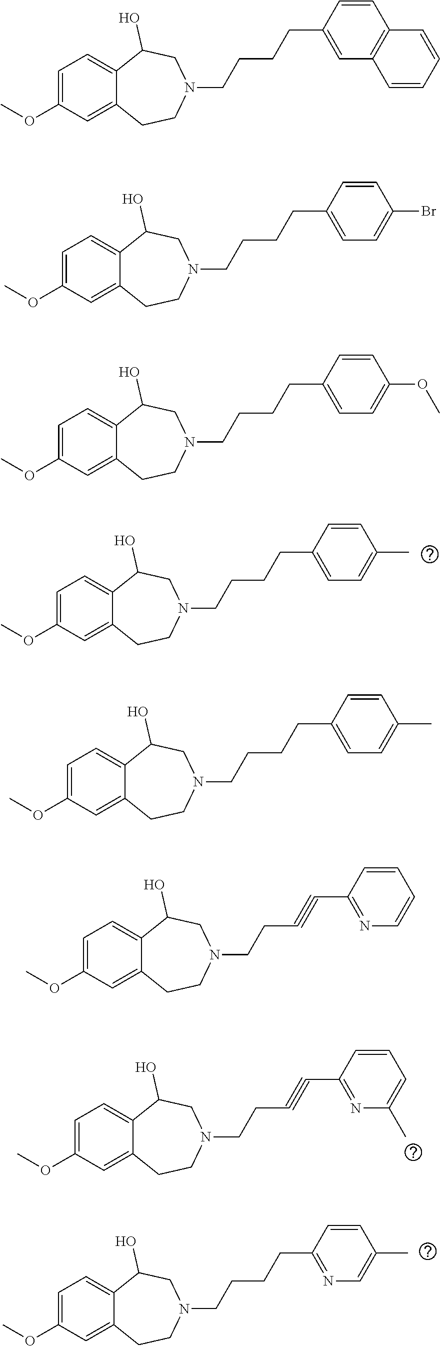

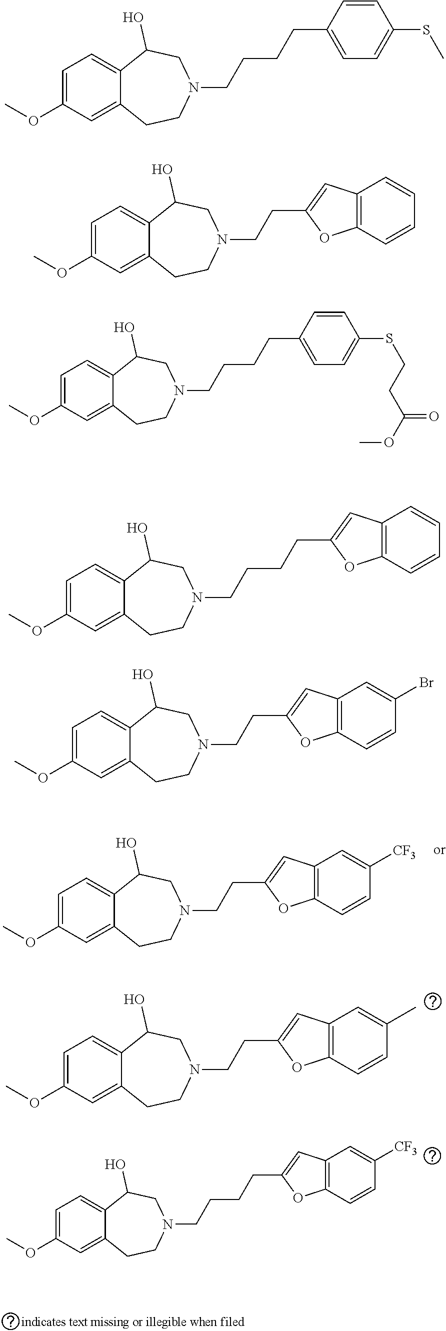

### Formula 205

In an embodiment, the NMDA receptor antagonist may be selected from one of the following compounds according to Formula 205. Such compounds are described in WO2019/152688, published Aug. 8, 2019 and corresponding to PCT/US2019/016114, filed Jan. 31, 2019, which is incorporated by reference in its entirety herein. In the case of any conflict of terminology of Formula 205, these references incorporated by reference herein control.

In an embodiment, the NMDA receptor antagonist is a compound according to Formula 205:

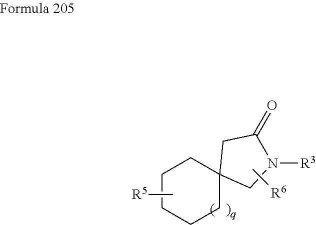

or a pharmaceutically acceptable salt and/or a stereoisomer thereof, wherein:
q is 1, 2, 3, or 4;
R5 is independently, for each occurrence, one or more optional substituents selected from the group consisting of C1-C6alkyl, —S(O)_{w}-C1-C3alkyl, —NRaRb, and halogen, wherein C1-C6alkyl is optionally substituted with one, two, or three substitutents each independently selected from Rs;
R6 is one or two optional substituents bound to one or more available ring carbons, and is independently, for each occurrence, selected from the group consisting of C1-C6alkyl, —S(O)_{w}-C1-C3alkyl, —NRaRb, and halogen, wherein C1-C6alkyl is optionally substituted with one, two, or three substitutents each independently selected from Rs;
w is 0, 1, or 2;
R3 is selected from the group consisting of H, phenyl, C1-C6alkyl, —C(O)R31, and —C(O)OR32, wherein C|1-C6, alkyl is optionally substituted with one, two, or three substitutents each independently selected from Rs;
R31 and R32 are each independently selected from the group consisting of H, C1-C6alkyl, —C3-C6cycloalkyl, and phenyl, wherein C1-C6alkyl is optionally substituted with one, two, or three substitutents each independently selected from Rs, and phenyl is optionally substituted with one, two, or three substitutents each independently selected from RT;
Ra and Rb are independently, for each occurrence, selected from the group consisting of H, phenyl, C1-C4alkyl, —C1-C4alkylene-phenyl, or Ra and Rb taken together with the nitrogen to which they are attached form a 4-6 membered heterocyclic ring, wherein phenyl is optionally substituted with one, two, or three substitutents each independently selected from hydroxyl, —C1-C3alkoxy, —C1-C3alkyl, and halogen, and
C1-C3alkyl is optionally substituted with one, two or three halogens;
Rs is independently, for each occurrence, selected from the group consisting of —C(O)NRaRb, —NRaRb, hydroxyl, S(O)w-C1-C3alkyl, SH, phenyl and halogen; and RT is independently, for each occurrence, selected from the group consisting of —C1-C6alkyl, hydroxyl, C1-C6alkoxy, and halogen.

In further embodiments, the NMDA receptor antagonist is a compound selected from:

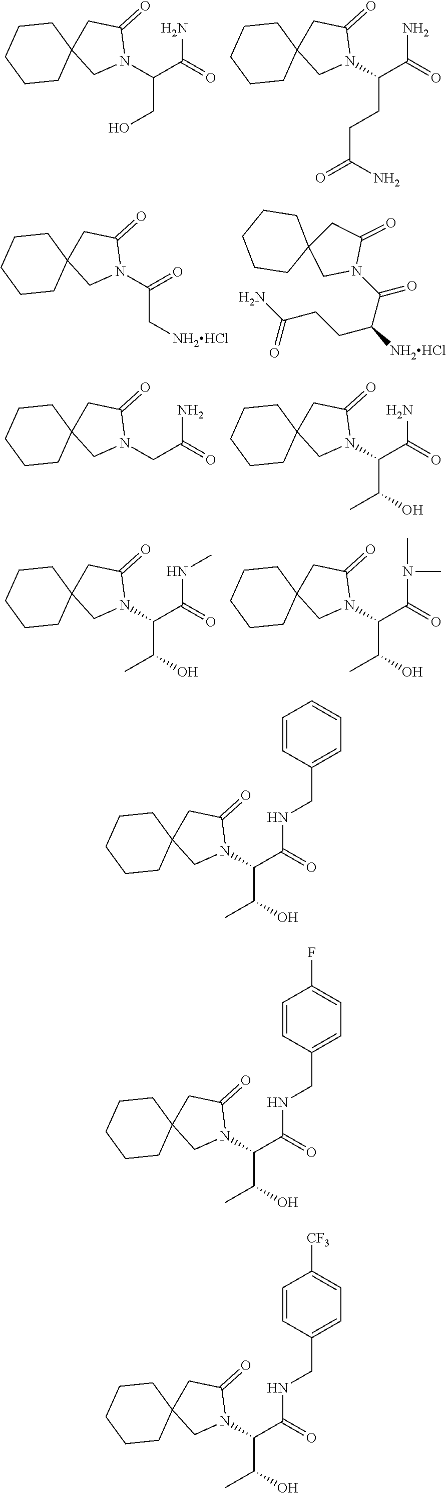

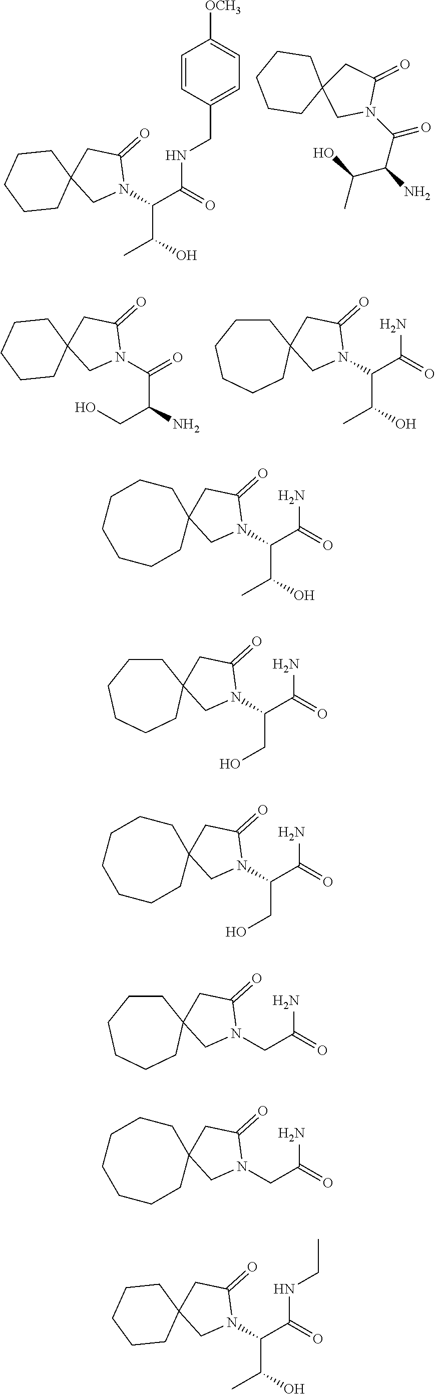

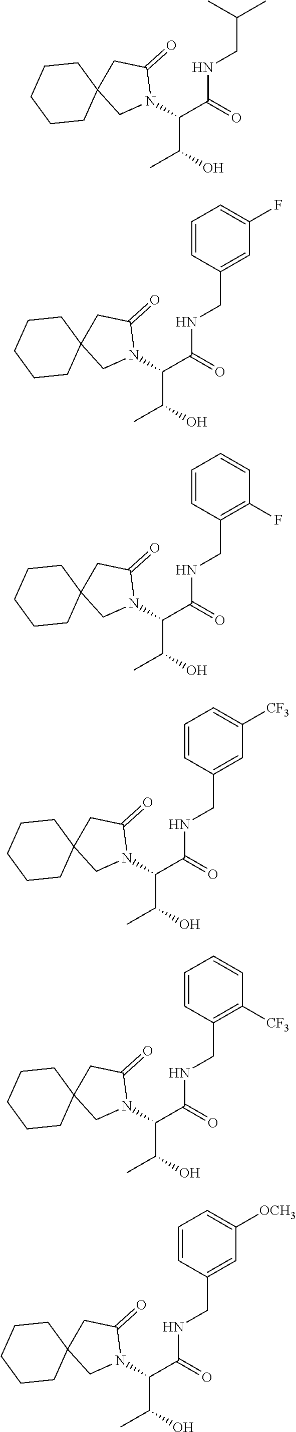

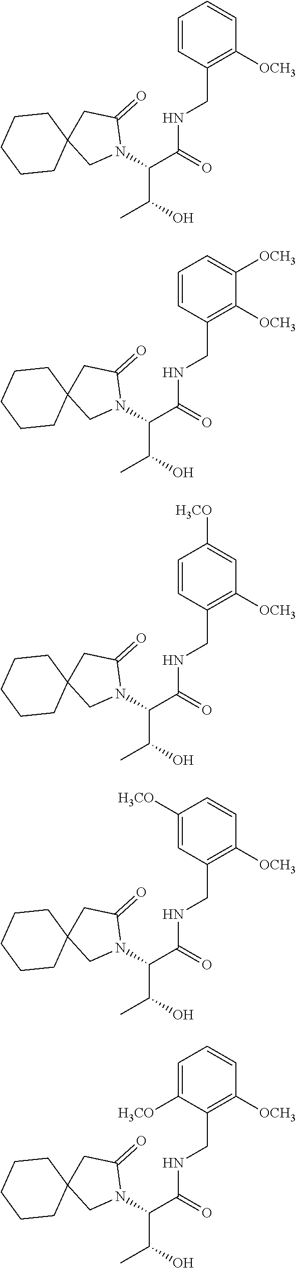

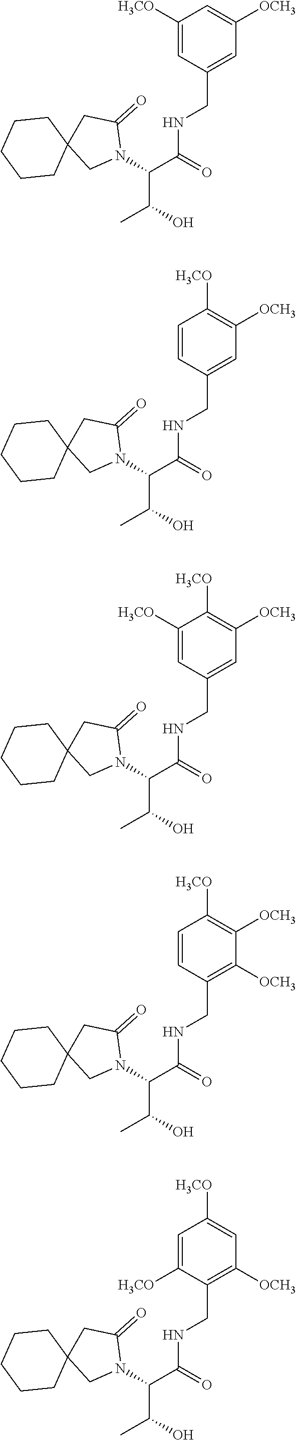 [SMILES: COc1cc(CNC(=O)[C@H]([C@@H](C)O)N2CC3(CCCCC3)CC2=O)cc(OC)c1 ; COc1ccc(CNC(=O)[C@H]([C@@H](C)O)N2CC3(CCCCC3)CC2=O)cc1OC ; COc1cc(CNC(=O)[C@H]([C@@H](C)O)N2CC3(CCCCC3)CC2=O)cc(OC)c1OC ; COc1ccc(CNC(=O)[C@H]([C@@H](C)O)N2CC3(CCCCC3)CC2=O)c(OC)c1OC ; COc1cc(OC)c(CNC(=O)[C@H]([C@@H](C)O)N2CC3(CCCCC3)CC2=O)c(OC)c1]

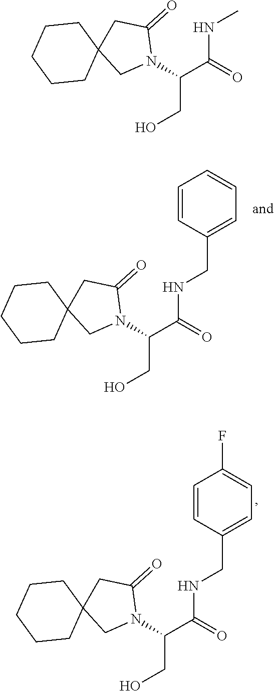 [SMILES: CNC(=O)[C@H](CO)N1CC2(CCCCC2)CC1=O ; O=C(NCc1ccccc1)[C@H](CO)N1CC2(CCCCC2)CC1=O ; O=C(NCc1ccc(F)cc1)[C@H](CO)N1CC2(CCCCC2)CC1=O]

including pharmaceutically acceptable salts thereof.

### Formula 206

In an embodiment, the NMDA receptor antagonist may be selected from one of the following compounds according to Formula 206. Such compounds are described in, which are each incorporated by reference in their entirety herein. In the case of any conflict of terminology of Formula 206, these references incorporated by reference herein control.

In an embodiment, the NMDA receptor antagonist may be a ketamine prodrug selected from:

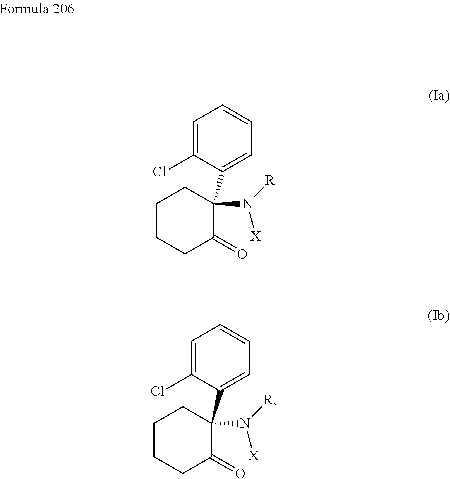

wherein R is —C(═O)R 1, —C(═O)OR 2, —C(═O)O(CHR 3) OC(═O)R 4 or —CD 3; and X is —CH 3 or —CD 3, including pharmaceutically acceptable salts thereof, or

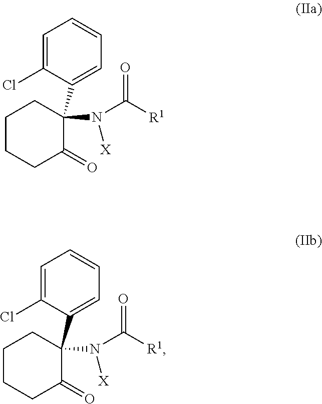

wherein R 1 is, optionally, substituted or unsubstituted aryl-OH, aryl-NH 2, alkenyl-OH, alkenyl-NH 2, alkyl-NH 2, alkyl-OH, carbocyclyl or heterocyclyl containing one or more N or O; and X is —CH 3 or —CD 3, including pharmaceutically acceptable salts thereof, or

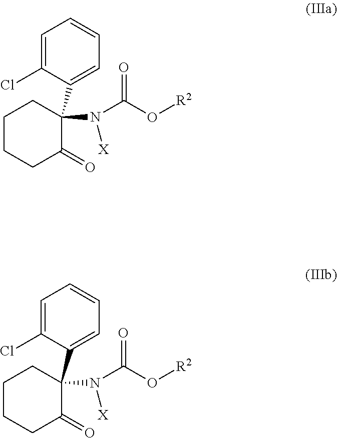

wherein R2 is optionally substituted or unsubstituted alkyl, aryl, carbocyclyl or heterocyclyl containing one or more O; and X is —CH 3 or —CD 3, including pharmaceutically acceptable salts thereof, or

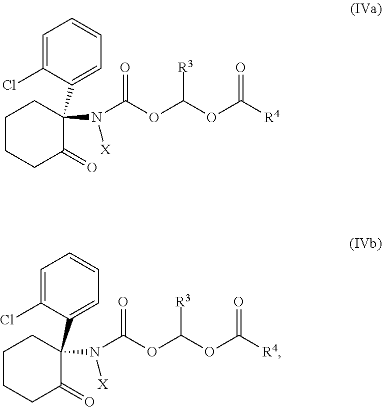

wherein R4 is independently substituted or unsubstituted alkyl, aryl, azaaryl, carbocyclyl or heterocyclyl containing one or more O or N, while R3 is H or substituted or unsubstituted alkyl; and X is —CH 3 or —CD 3, including pharmaceutically acceptable salts thereof. In further embodiments, the NMDA receptor antagonist is a compound selected from:

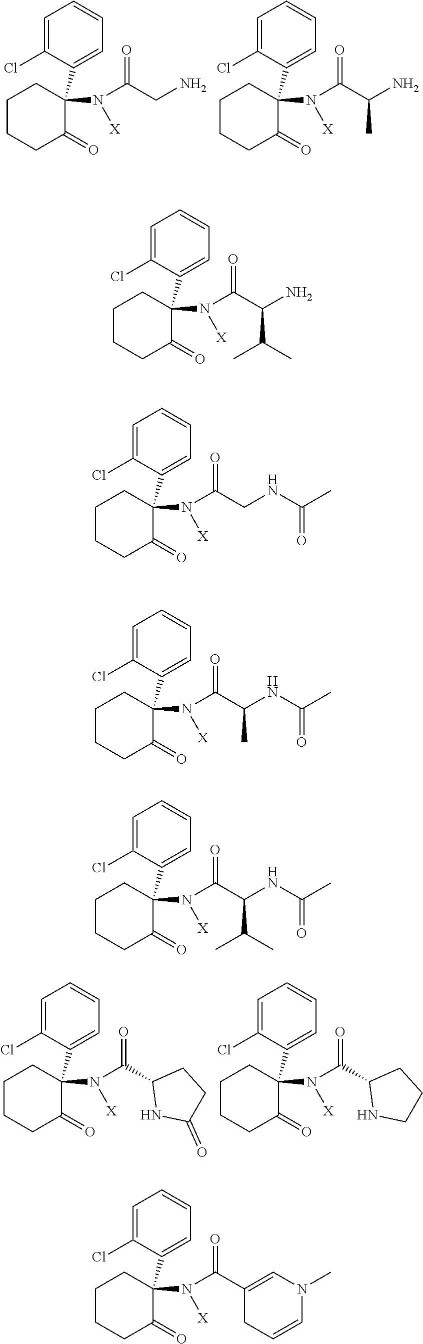

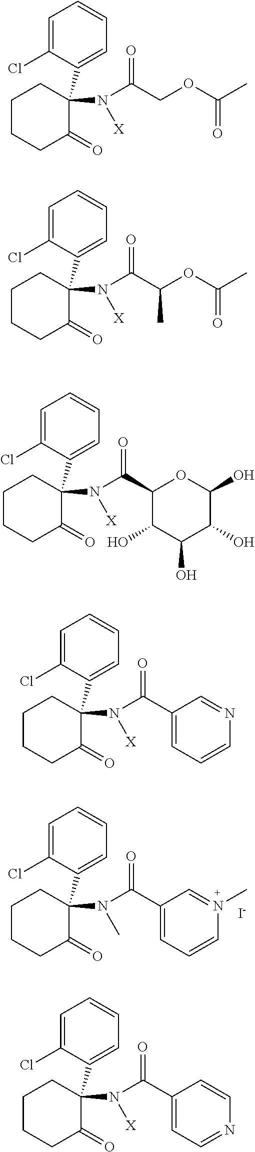

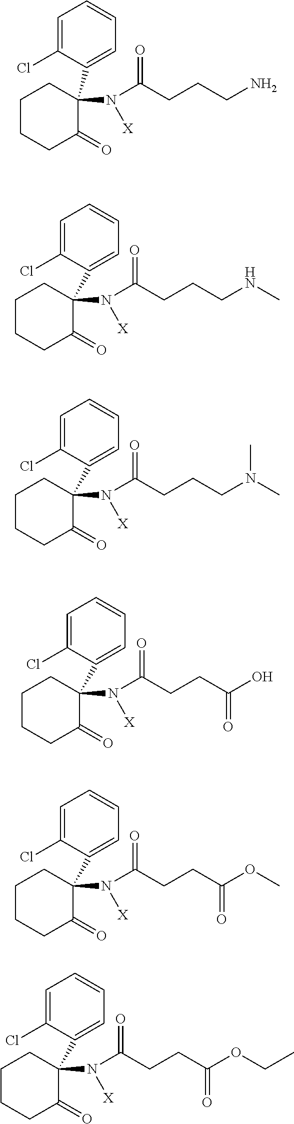

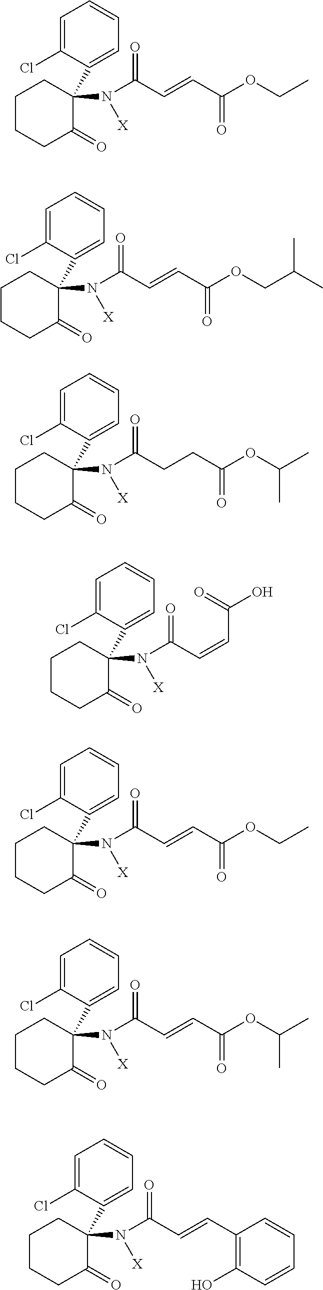

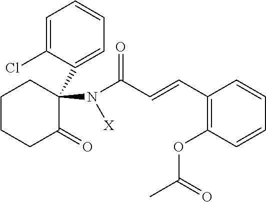

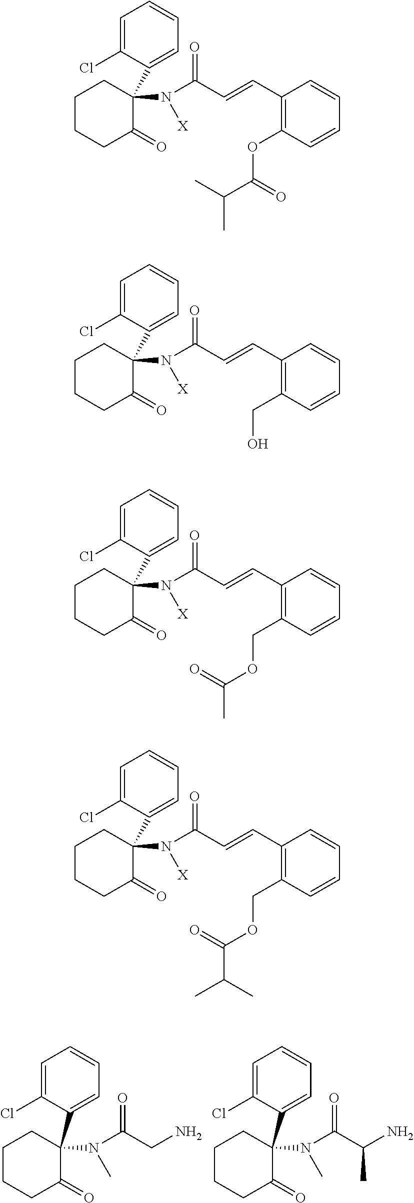

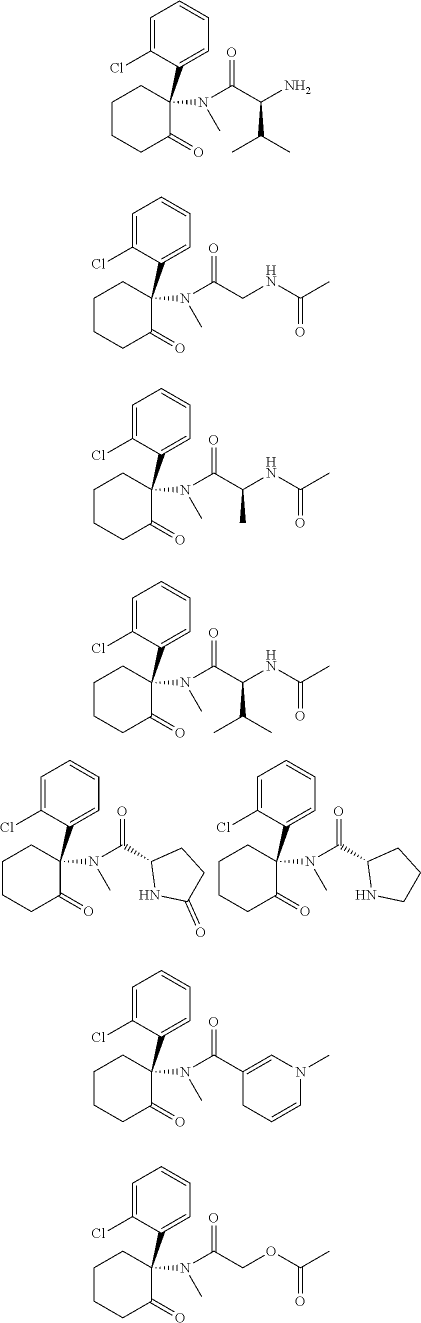

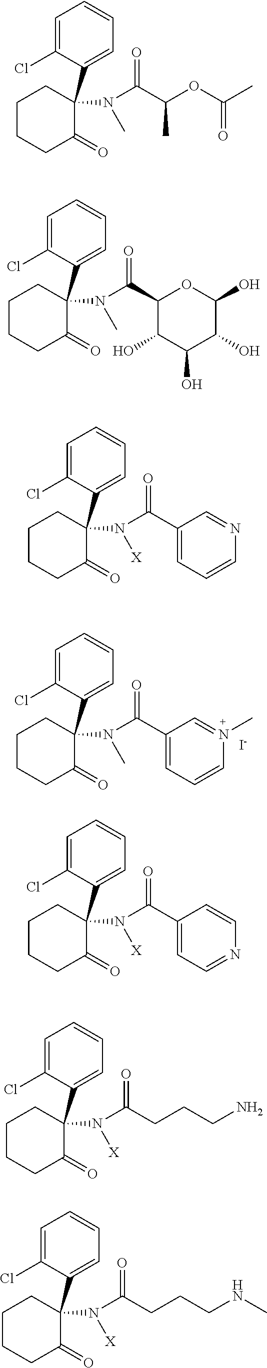

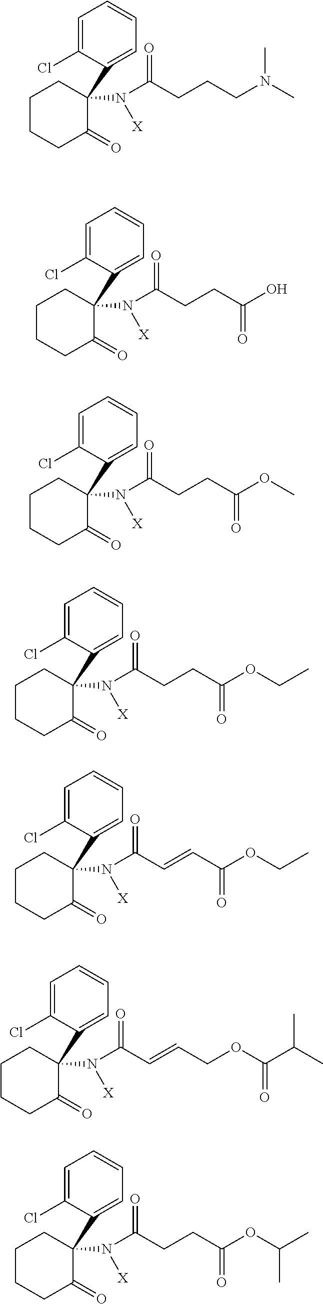

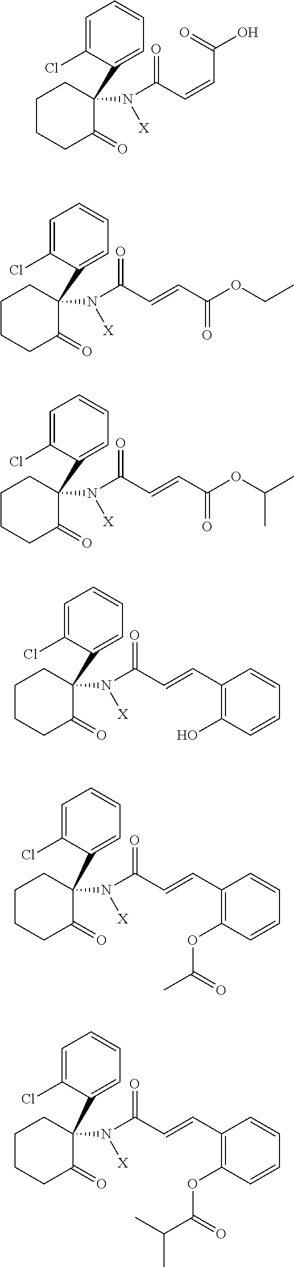

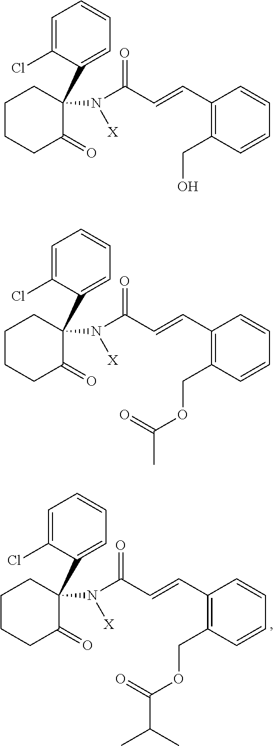

Wherein X is —CH₃ or —CD3, including pharmaceutically acceptable salts thereof.

In further embodiments, the NMDA receptor antagonist is a compound selected from:

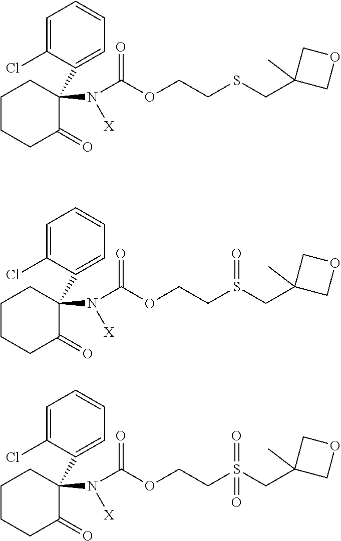

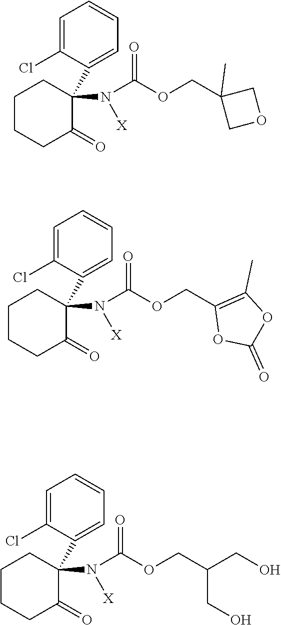

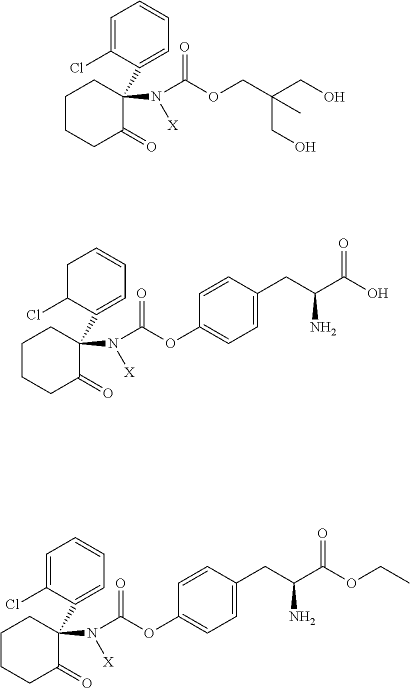

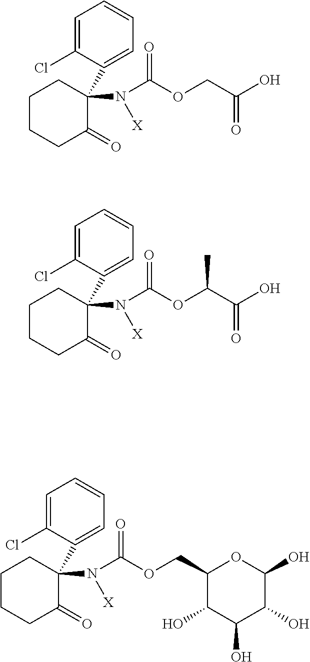

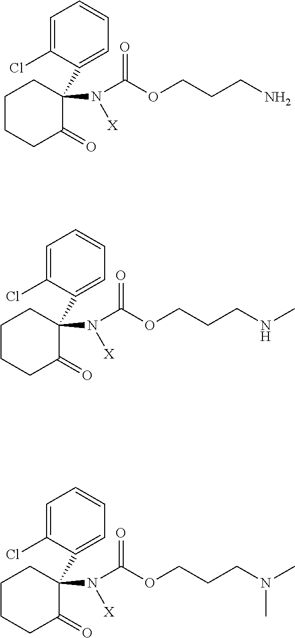

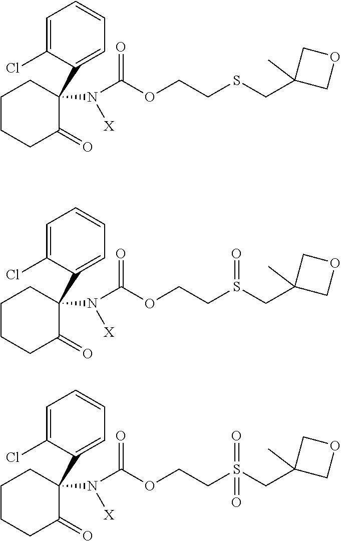

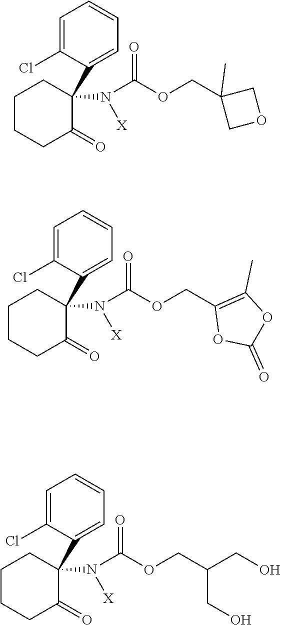

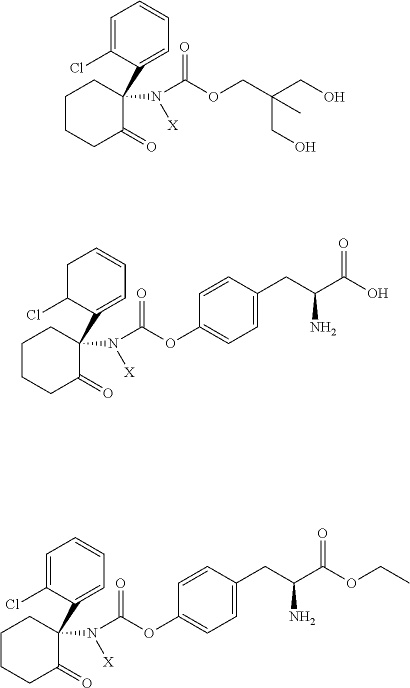

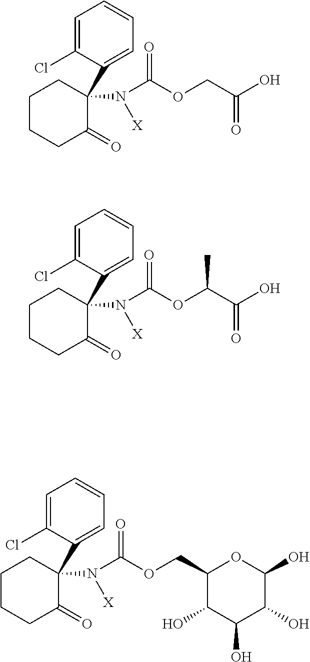

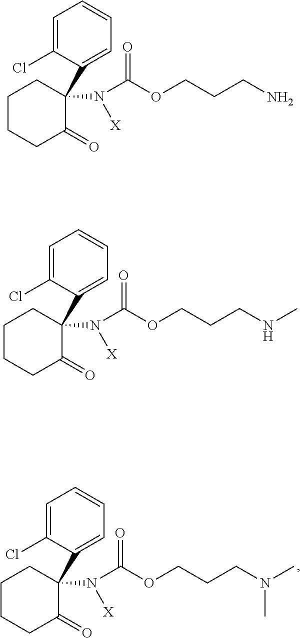

including pharmaceutically acceptable salts thereof

In further embodiments, the NMDA receptor antagonist is selected from:

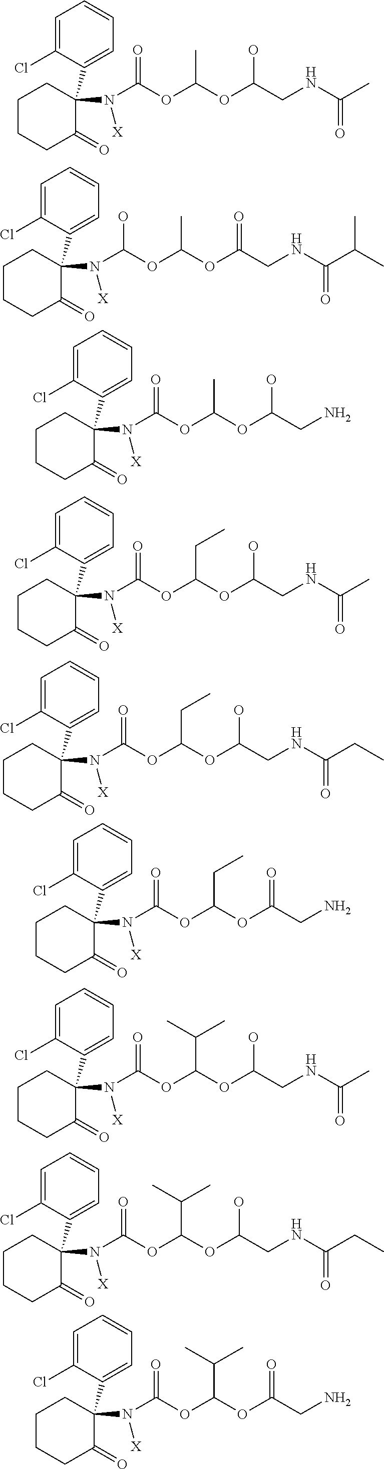

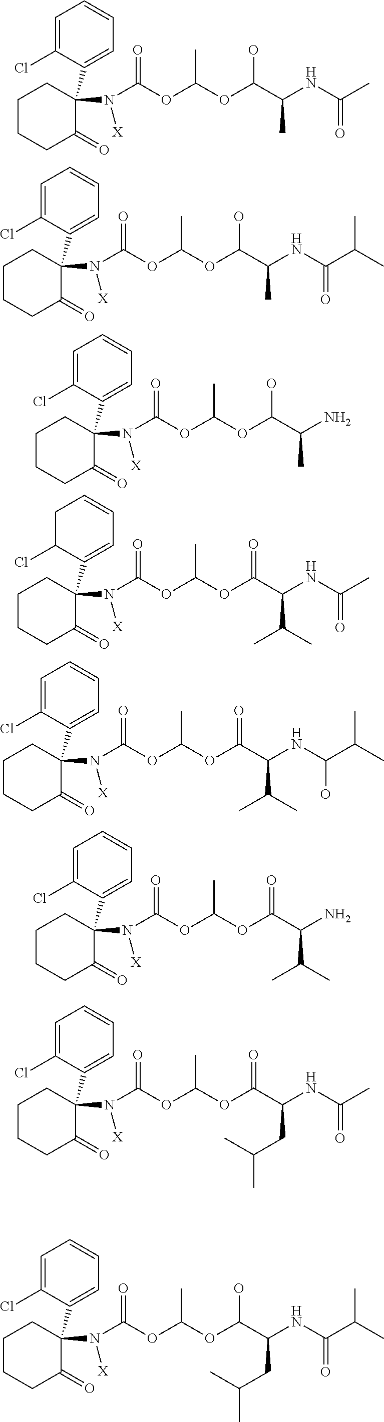

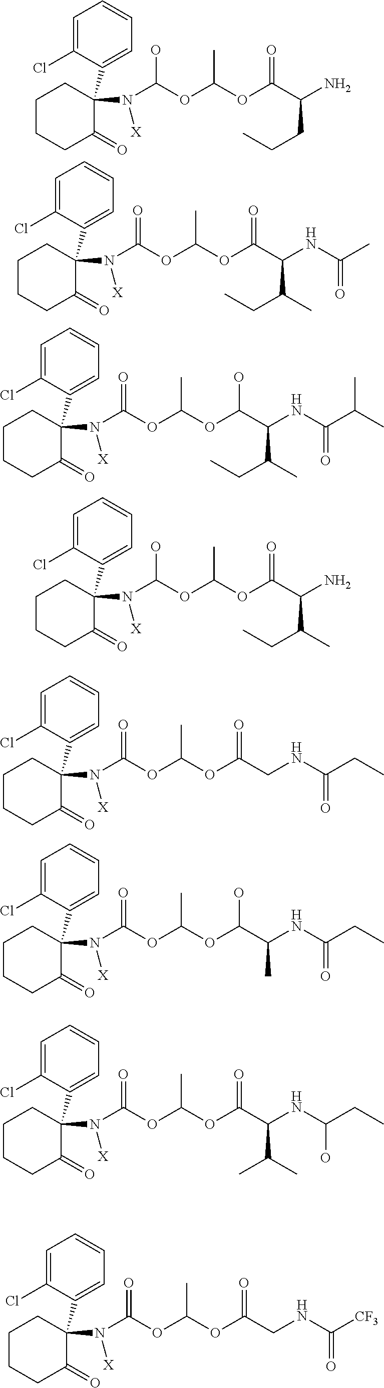

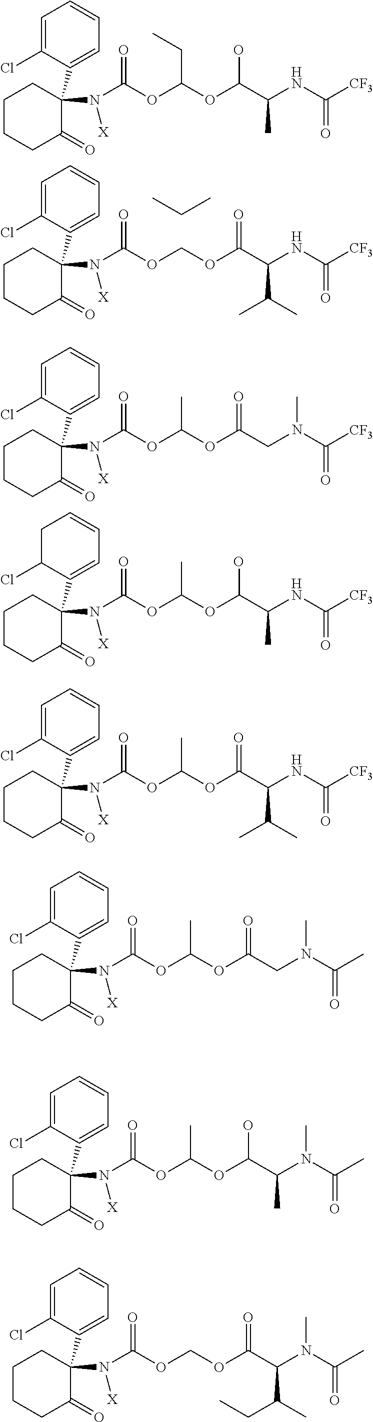

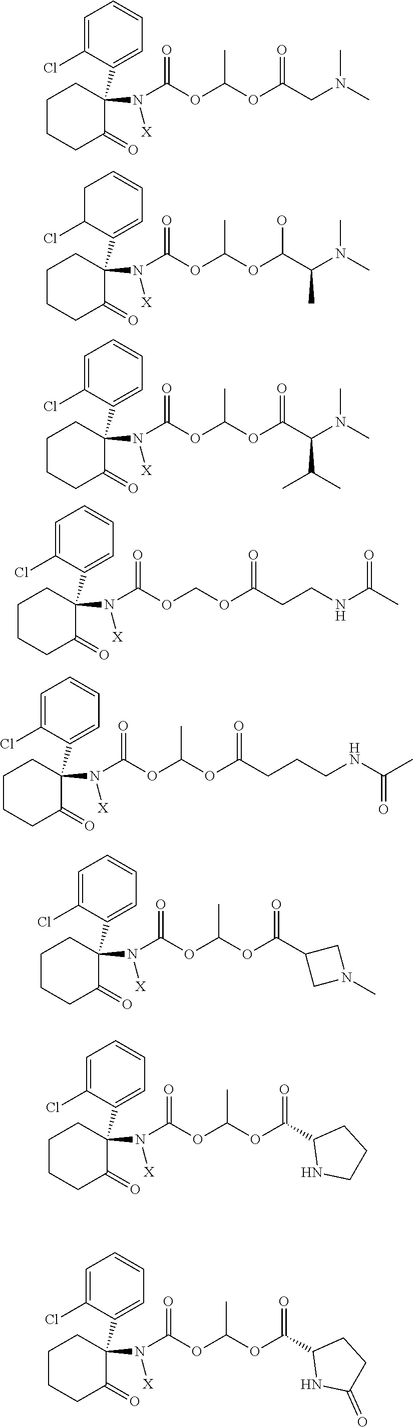

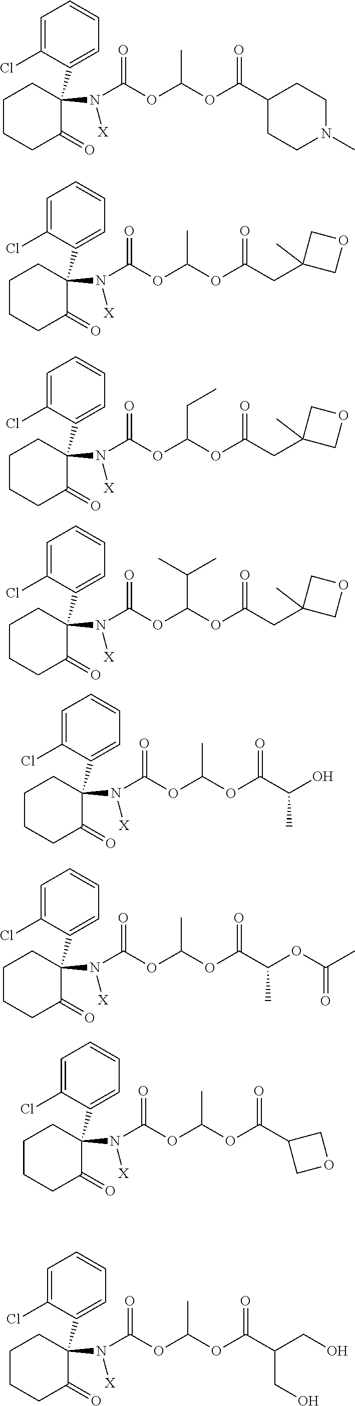

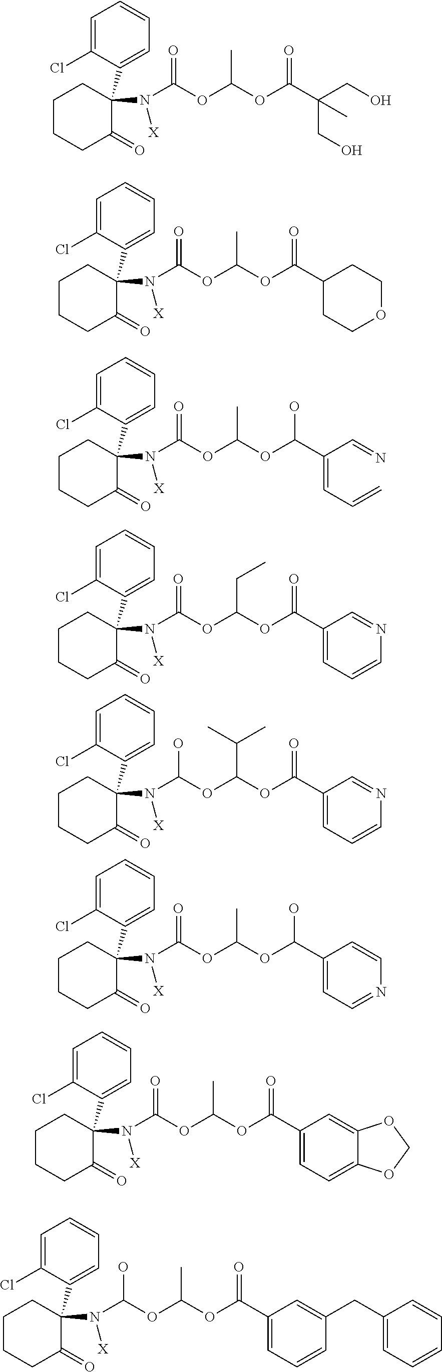

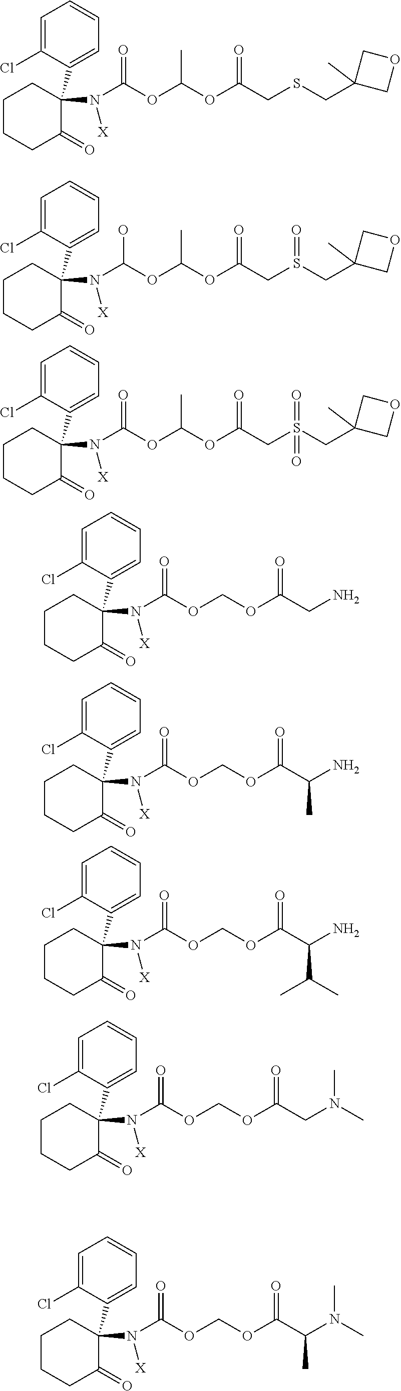

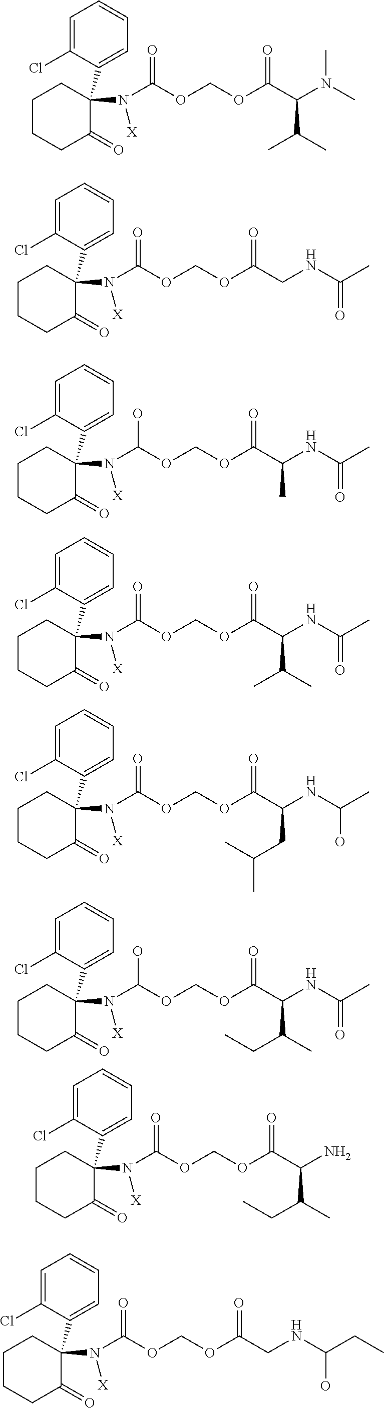

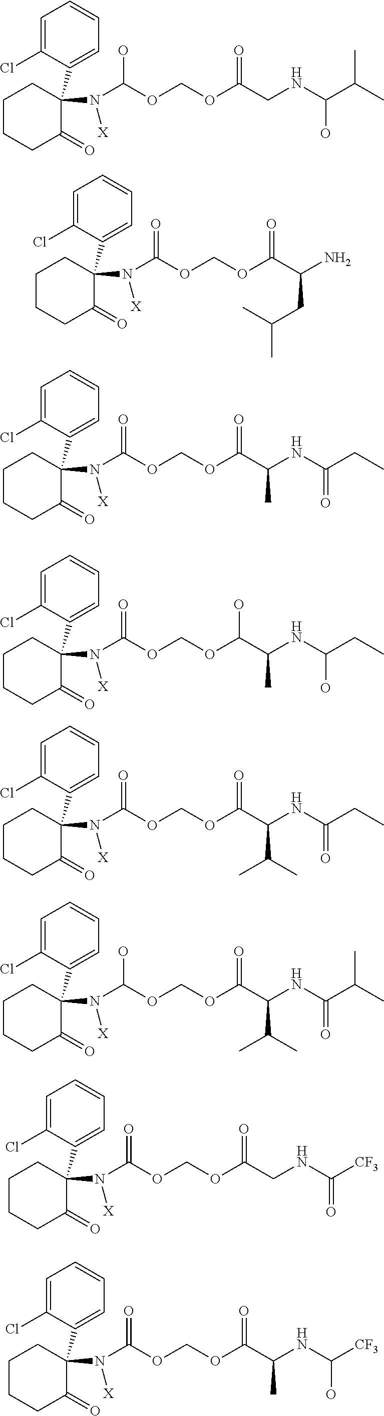

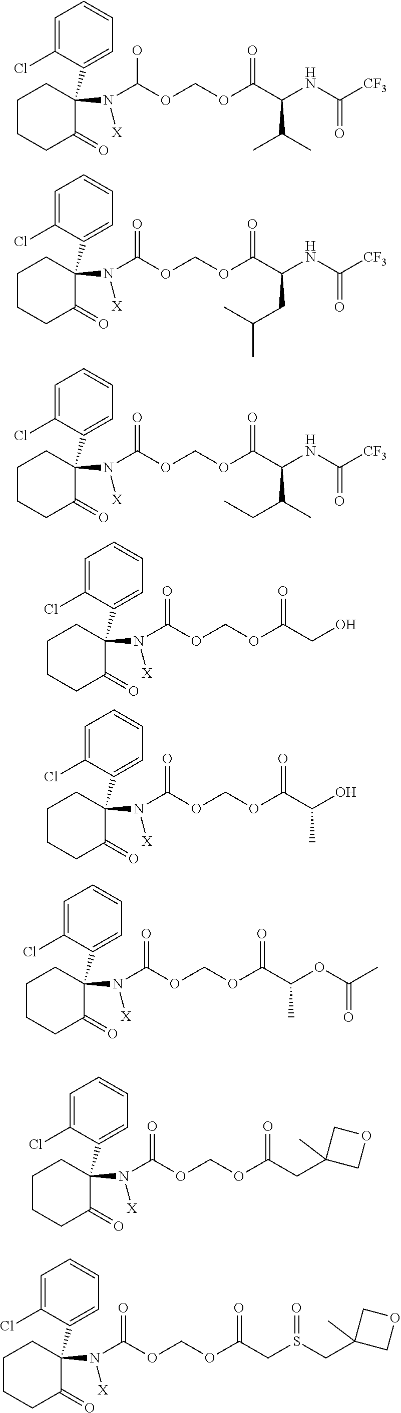

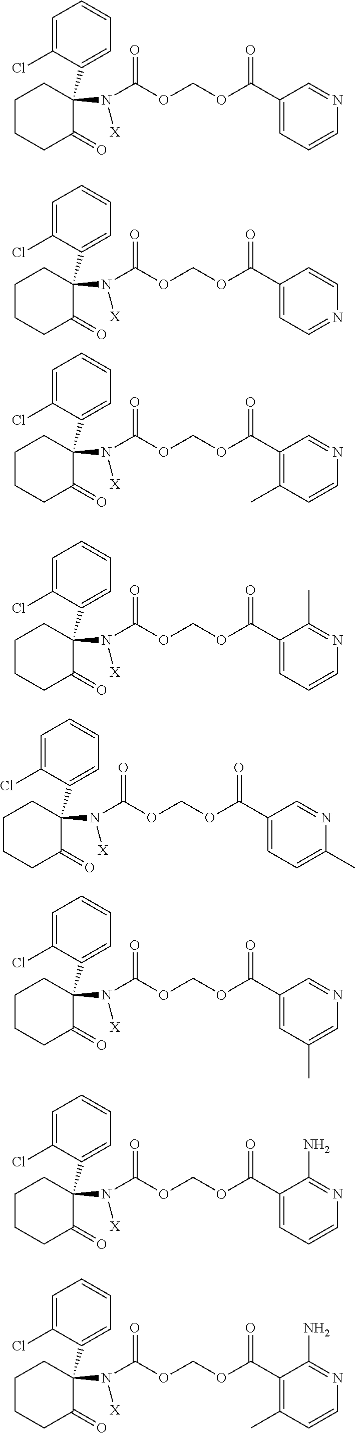

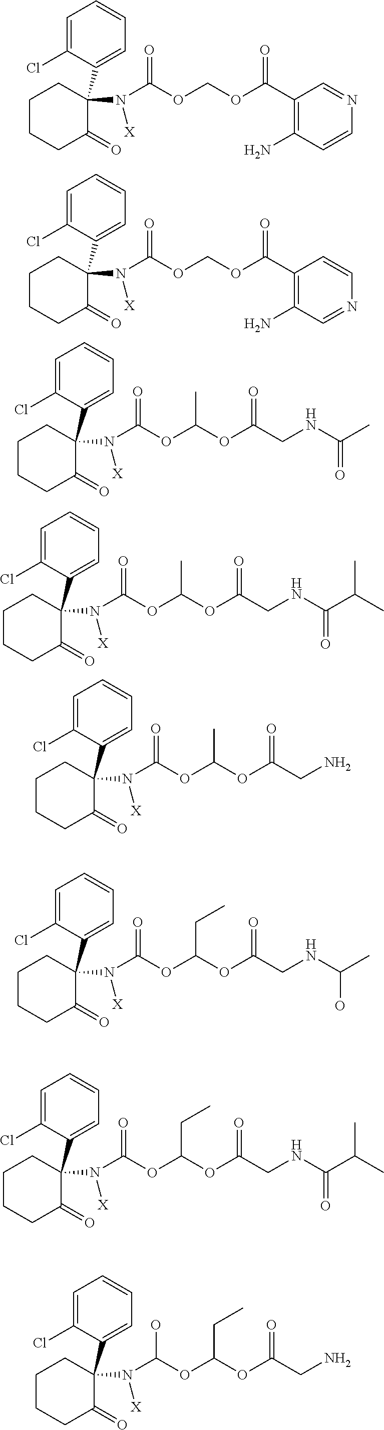

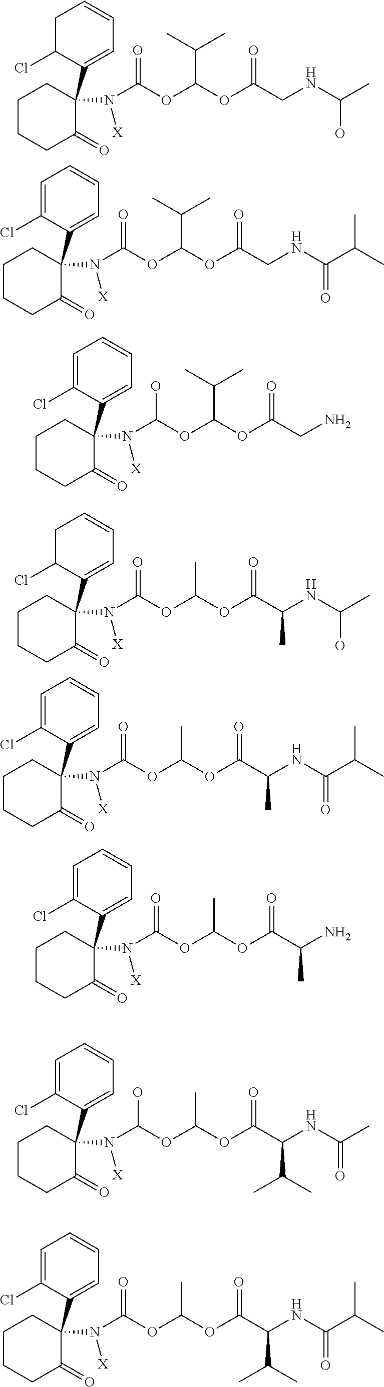

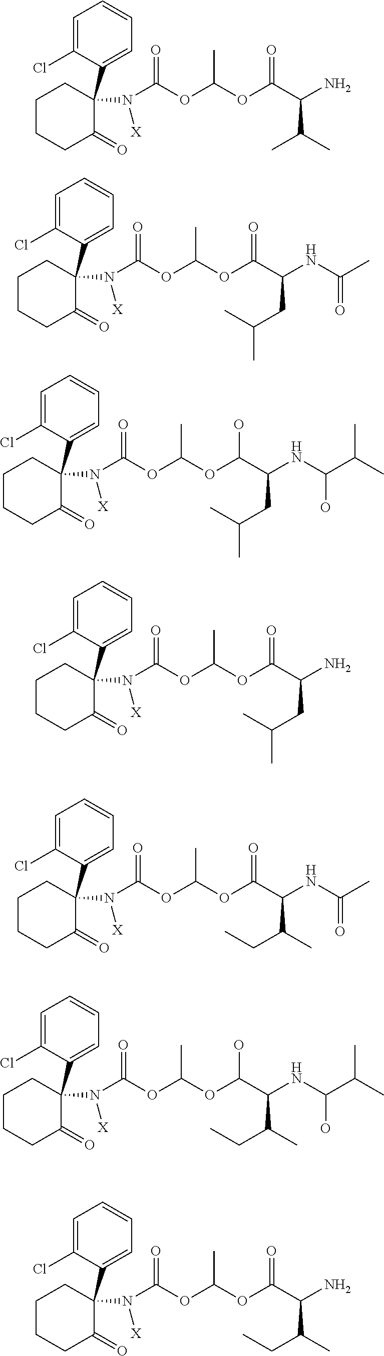

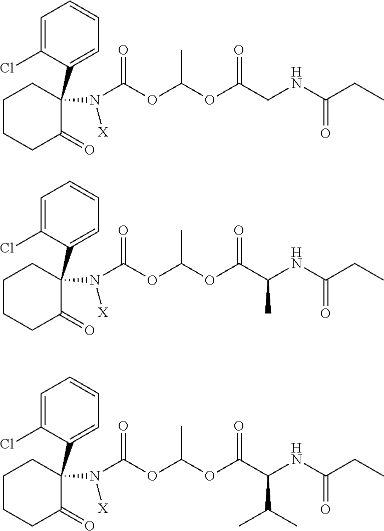

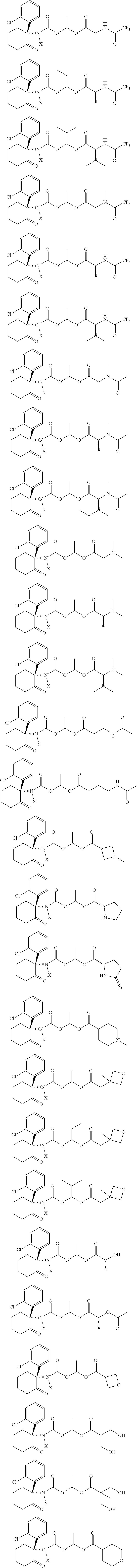

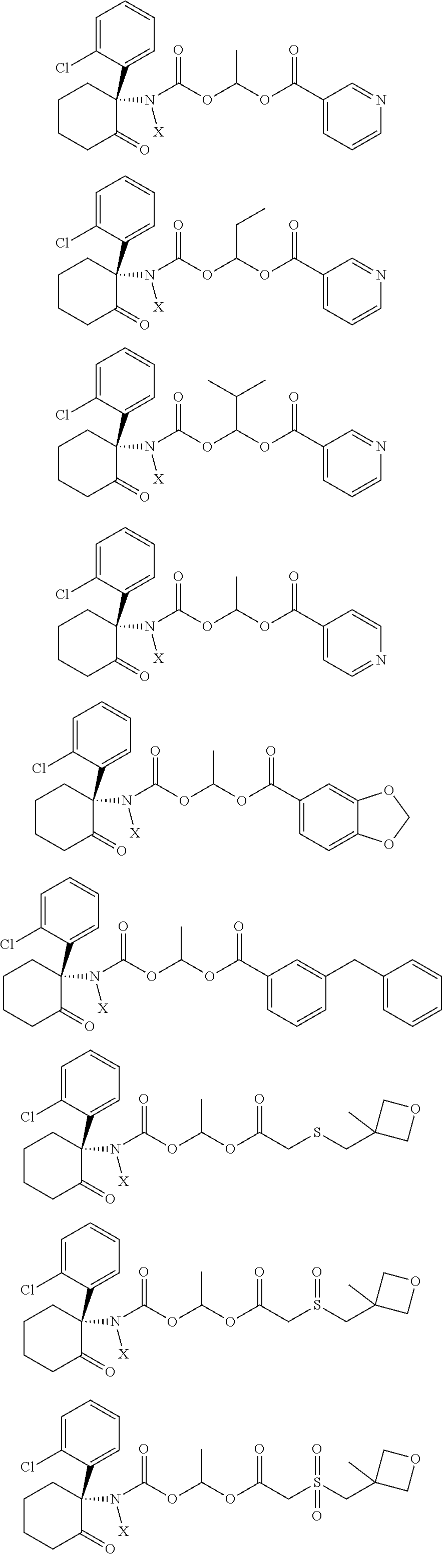

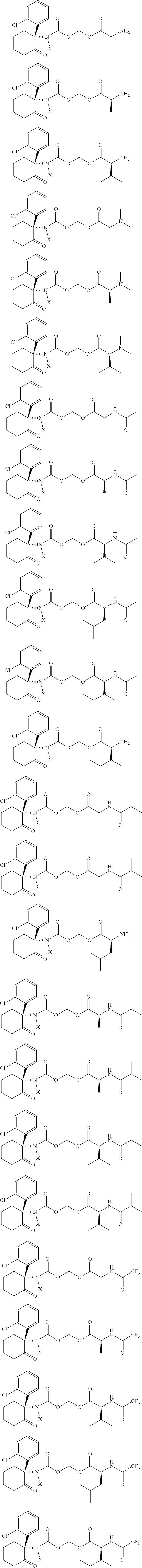

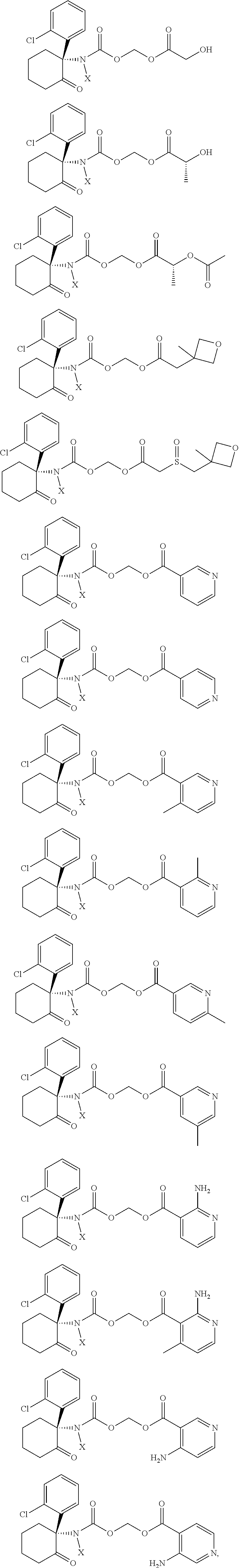

wherein X is —CH₃ or —CD3, including pharmaceutically acceptable salts thereof.

In further embodiments, the NMDA receptor antagonist is selected from:

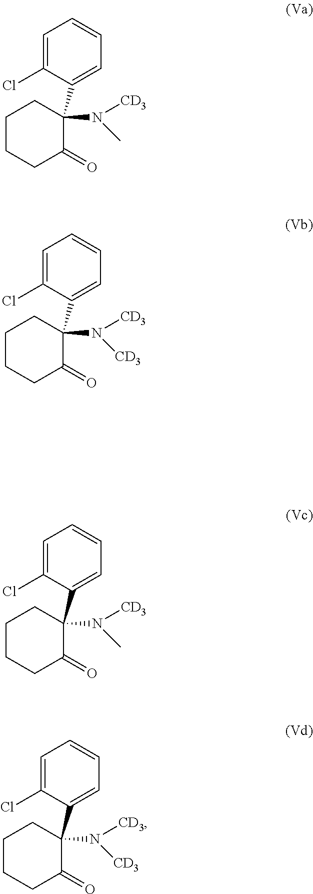 [SMILES: [2H]C([2H])([2H])N(C)[C@]1(c2ccccc2Cl)CCCCC1=O ; [2H]C([2H])([2H])N(C([2H])([2H])[2H])[C@]1(c2ccccc2Cl)CCCCC1=O ; [2H]C([2H])([2H])N(C)[C@@]1(c2ccccc2Cl)CCCCC1=O ; [2H]C([2H])([2H])N(C([2H])([2H])[2H])[C@@]1(c2ccccc2Cl)CCCCC1=O]

including pharmaceutically acceptable salts thereof.

### Formula 207

In an embodiment, the NMDA receptor antagonist may be selected from one of the following compounds according to Formula 207. Such compounds are described in WO2018/075699, published Apr. 26, 2018 and corresponding to PCT/US2017/057277, filed Oct. 18, 2017, and U.S. Pat. No. 11,149,054, issued Oct. 19, 2021, which are each incorporated by reference in their entirety herein. In the case of any conflict of terminology of Formula 207, these references incorporated by reference herein control.

In an embodiment, the NMDA receptor antagonist is a compound according to Formula 207:

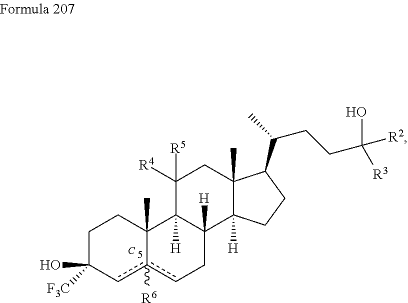

or a pharmaceutically acceptable salt thereof, wherein: each of R2 and R3 is independently hydrogen, substituted or unsubstituted C1-C6 alkyl, substituted or unsubstituted carbocyclyl, or substituted or unsubstituted heterocyclyl, or R2 and R3, together with the carbon atom to which they are attached form a substituted or unsubstituted 3-8 membered ring; each of R4 and R5 is independently hydrogen, halo, or —ORc, wherein Rc is hydrogen or substituted or unsubstituted C1-C6 alkyl, or R4 and R5, together with the carbon atom to which they are attached form an oxo group; R6 is absent or hydrogen; and represents a single or double bond, wherein when one of is a double bond, the other is a single bond; when both of are single bonds, then R is hydrogen; and when one of is a double bond, R is absent, or

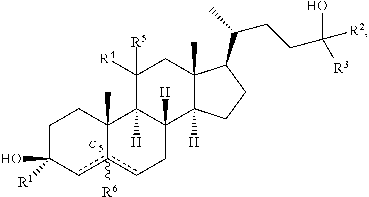

or a pharmaceutically acceptable salt thereof, wherein: R1 is substituted or unsubstituted C1-C6 alkyl; R2 is substituted or unsubstituted aralkyl, substituted or unsubstituted heteroaralkyl, substituted or unsubstituted aryl, or substituted or unsubstituted heteroaryl; R3 is hydrogen, substituted or unsubstituted C1-C6 alkyl, substituted or unsubstituted carbocyclyl, substituted or unsubstituted heterocyclyl, aryl, or substituted or unsubstituted heteroaryl; each of R4 and R5 is independently hydrogen, halo, or —ORc, wherein Rc is hydrogen or unsubstituted or substituted C1-C3 alkyl, or R4 and R5, together with the carbon atom to which they are attached form an oxo group; R6 is absent or hydrogen; and represents a single or double bond, wherein when one of is a double bond, the other is a single bond; when both of are single bonds, then R6 is hydrogen; and when one of is a double bond, R6 is absent, or

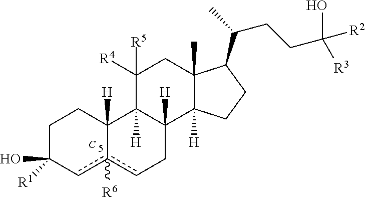

or a pharmaceutically acceptable salt thereof, wherein: R1 is hydrogen or substituted or unsubstituted C1-C6 alkyl; each of R2 and R3 is independently hydrogen, substituted or unsubstituted alkyl, substituted or unsubstituted aryl, substituted or unsubstituted heteroaryl, substituted or unsubstituted carbocyclyl, or substituted or unsubstituted heterocyclyl or R2 and R3, together with the carbon atom to which they are attached for a 3-8 substituted or unsubstituted membered ring; each of R4 and R5 is independently hydrogen, halo, or —ORc, wherein Rc is hydrogen or substituted or unsubstituted C1-C6 alkyl, or R4 and R5, together with the carbon atom to which they are attached form an oxo group; R6 is absent or hydrogen; and represents a single or double bond, wherein when one of is a double bond, the other is a single bond; when both of are single bonds, then R6 is hydrogen; and when one of is a double bond, R6 is absent, or

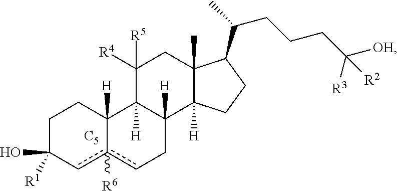

or a pharmaceutically acceptable salt thereof, wherein, R1 is hydrogen or substituted or unsubstituted C1-C6 alkyl; each of R2 and R3 is independently hydrogen, substituted or unsubstituted alkyl, substituted or unsubstituted carbocyclyl, or substituted or unsubstituted heterocyclyl or R2 and R3, together with the carbon atom to which they are attached, form a 3-8 substituted or unsubstituted membered ring; each of R4 and R5 is independently hydrogen, halo, or —ORc, wherein Rc is hydrogen or substituted or unsubstituted C1-C6 alkyl, or R4 and R5, together with the carbon atom to which they are attached form an oxo group; R6 is absent or hydrogen; and represents a single or double bond, wherein when one of is a double bond, the other is a single bond; when both of are single bonds, then R6 is hydrogen; and when one of is a double bond, R6 is absent.

In further embodiments, the NMDA receptor antagonist is a compound selected from:

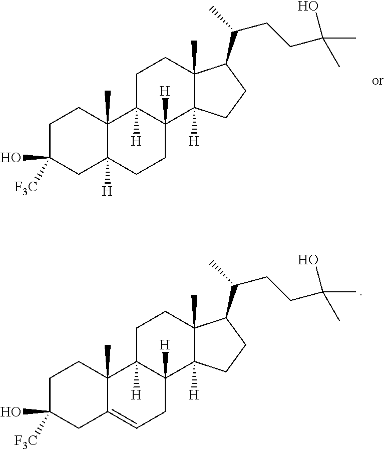

In further embodiments, the NMDA receptor antagonist is a compound selected from:

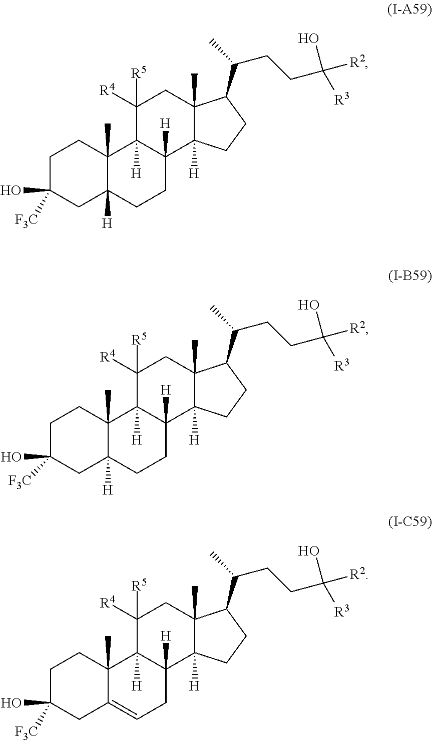

In further embodiments, the NMDA receptor antagonist is a compound selected from:

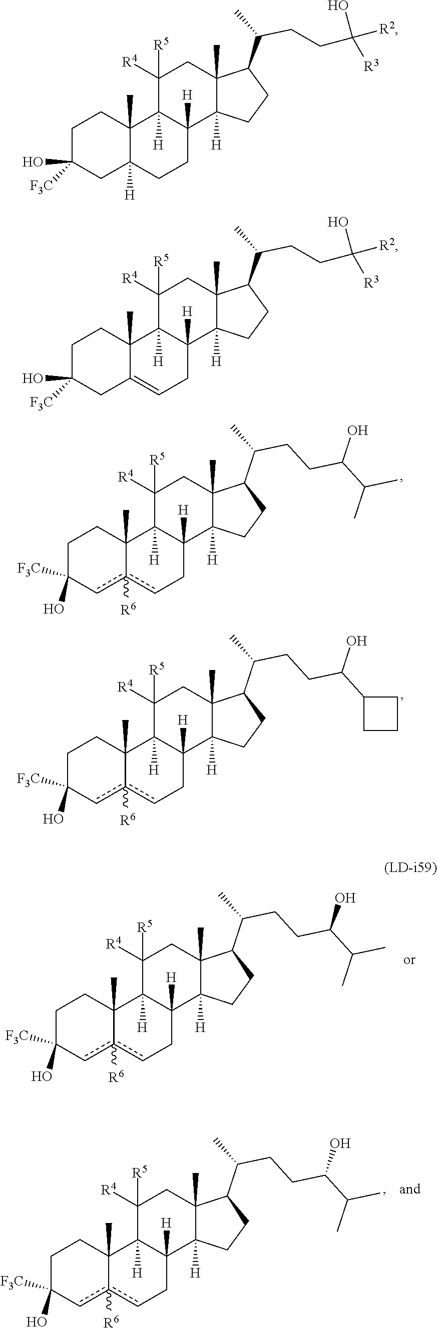

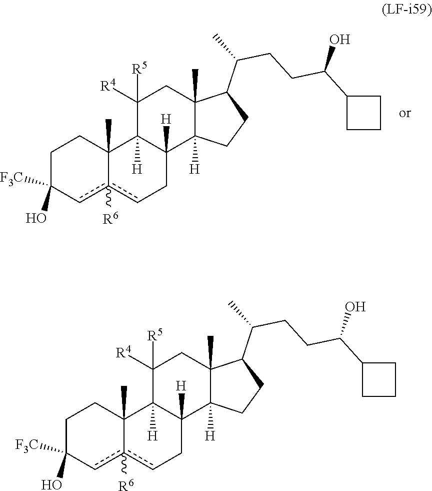

In further embodiments, the NMDA receptor antagonist is a compound selected from:

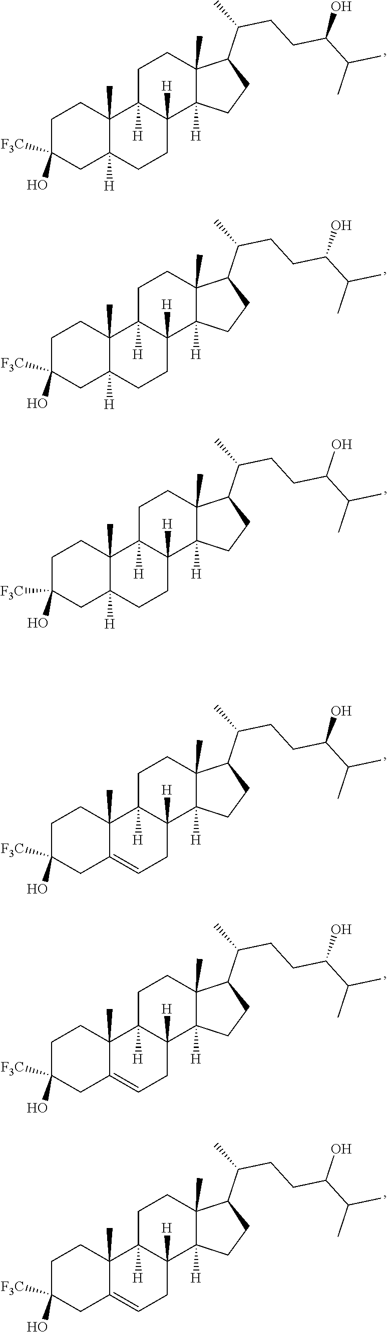

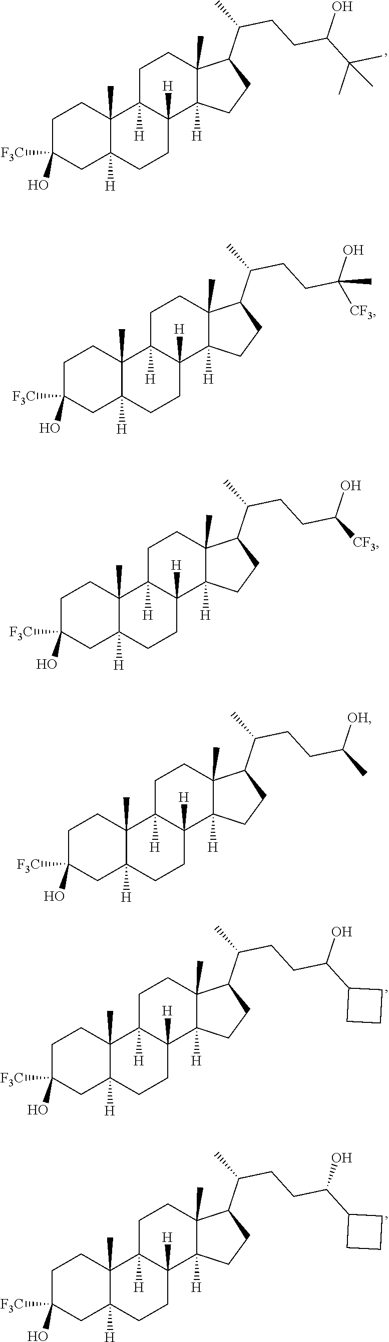

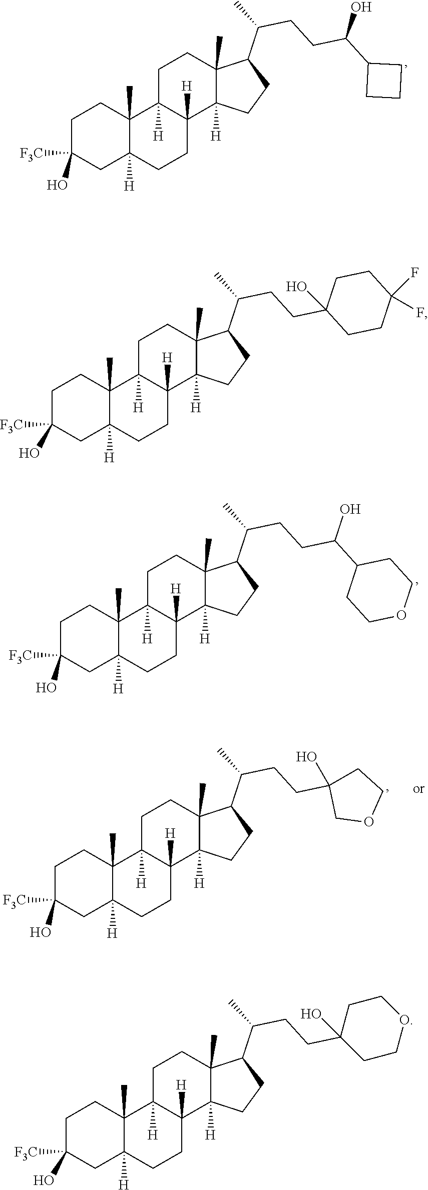

In further embodiments, the NMDA receptor antagonist is a compound selected from:

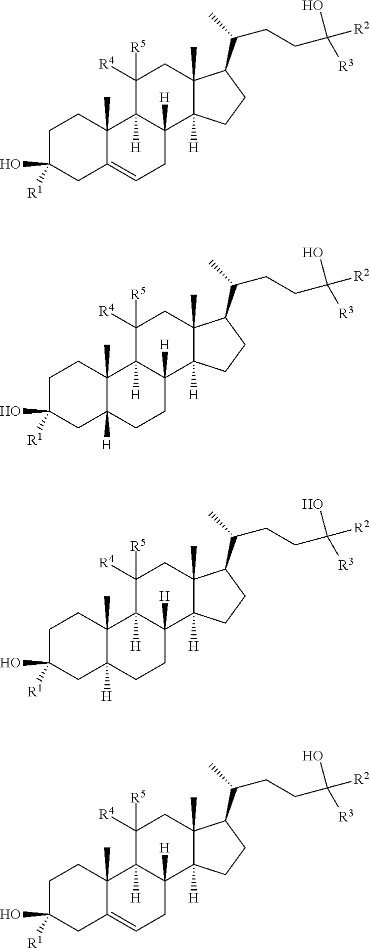

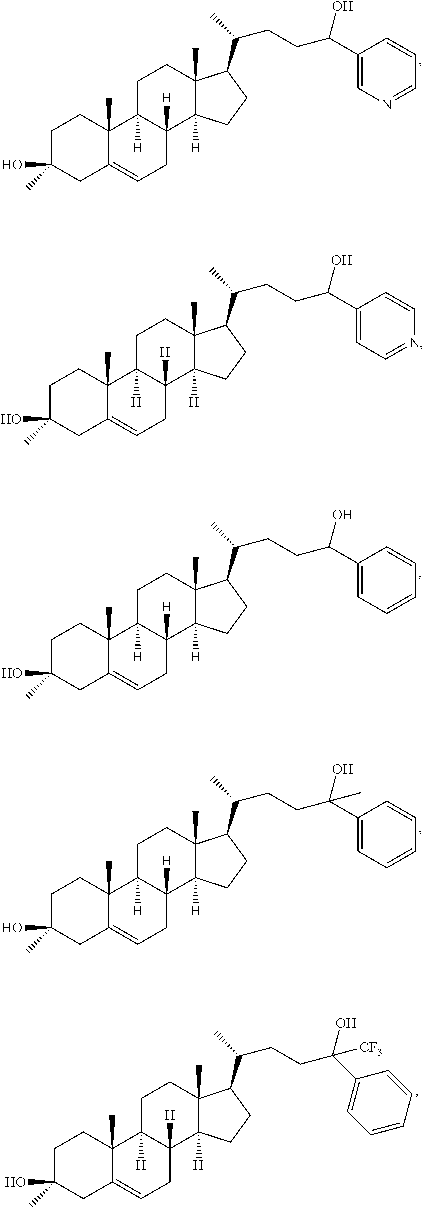

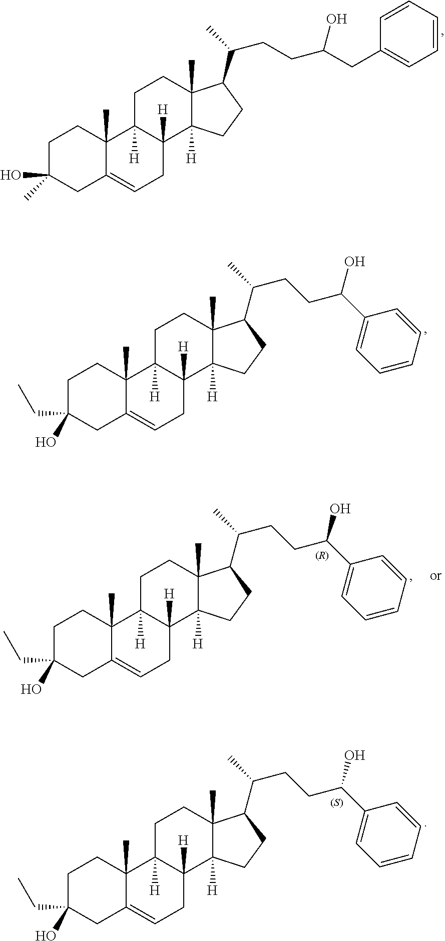

In further embodiments, the NMDA receptor antagonist is a compound selected from:

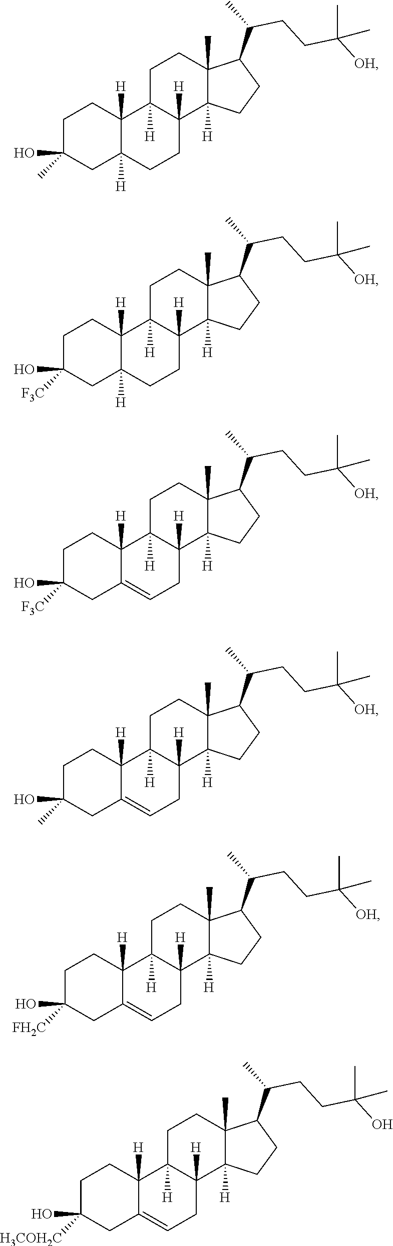

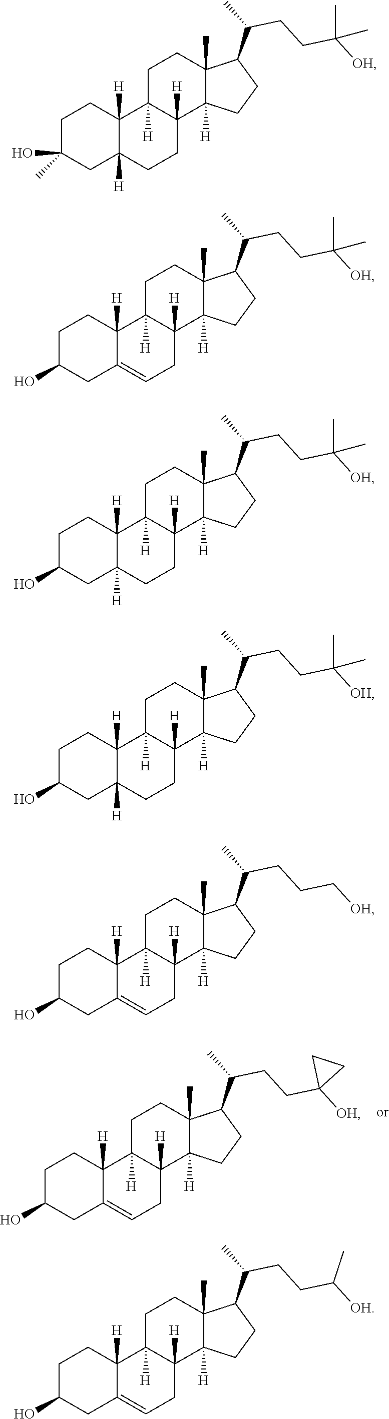

In further embodiments, the NMDA receptor antagonist is a compound selected from:

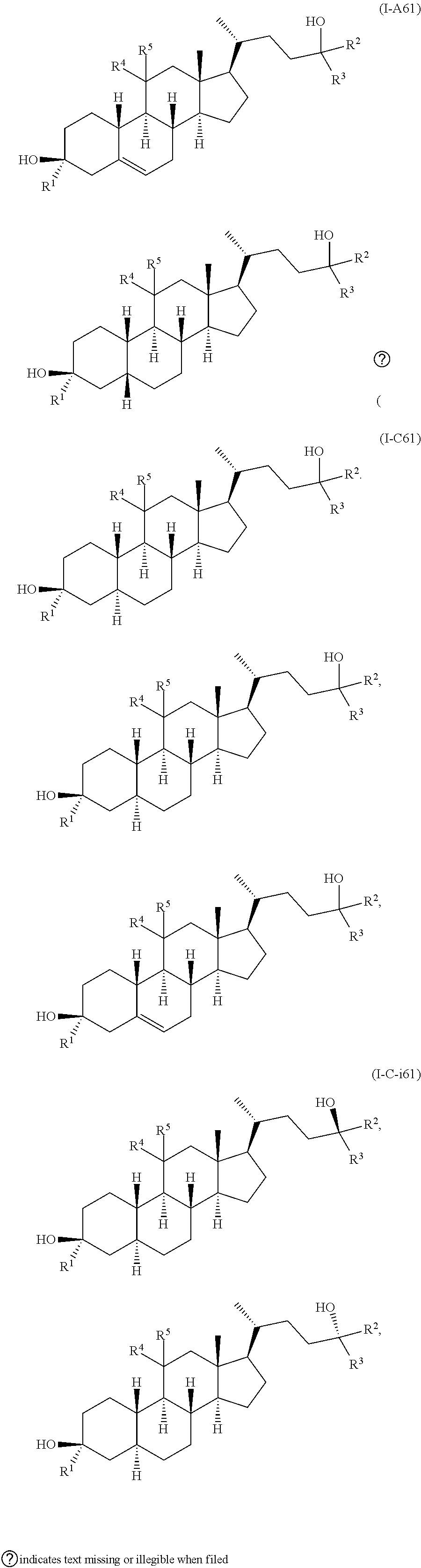

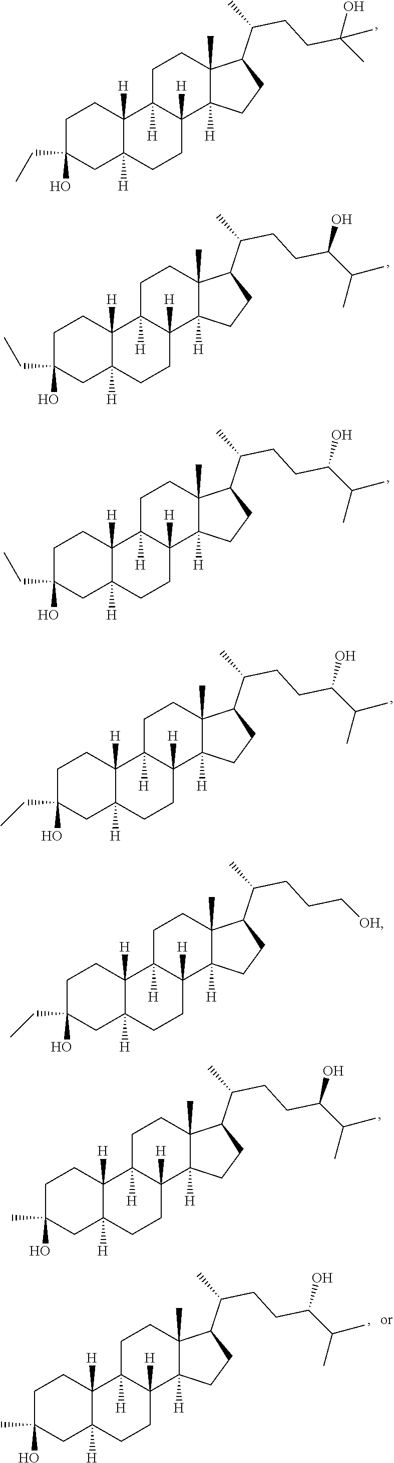

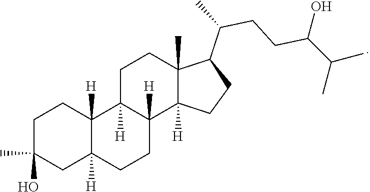

In further embodiments, the NMDA receptor antagonist is a compound selected from:

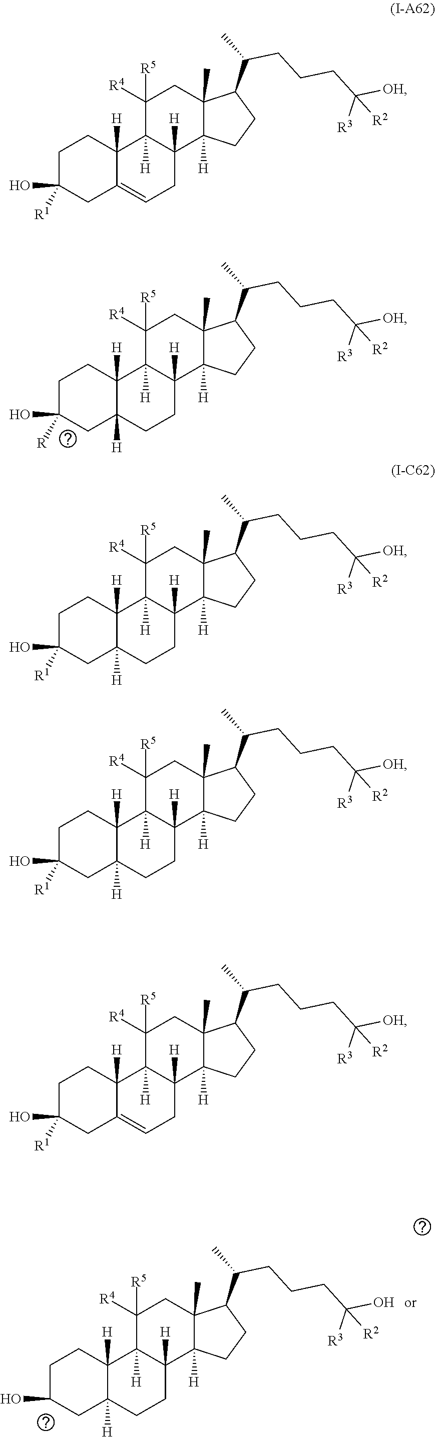

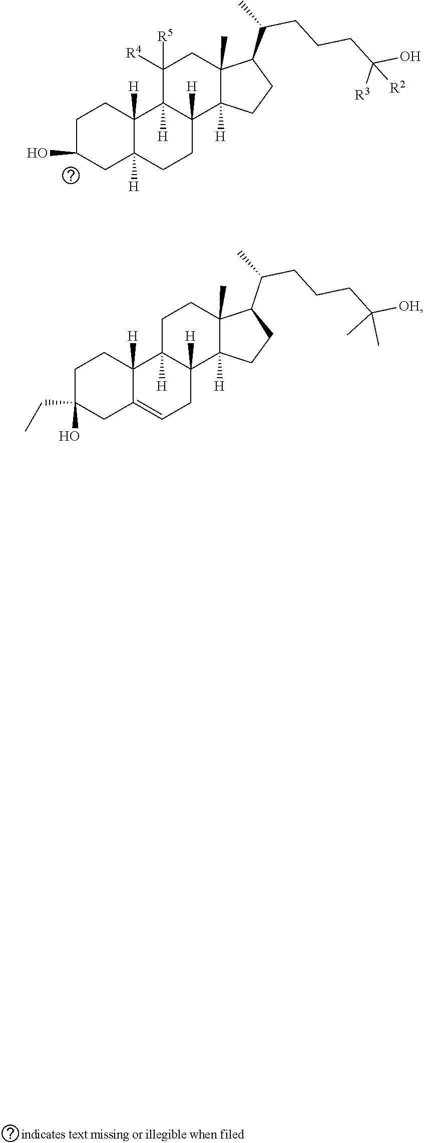

compound of Formula (I-A60), (I-B60), or (I-C60):

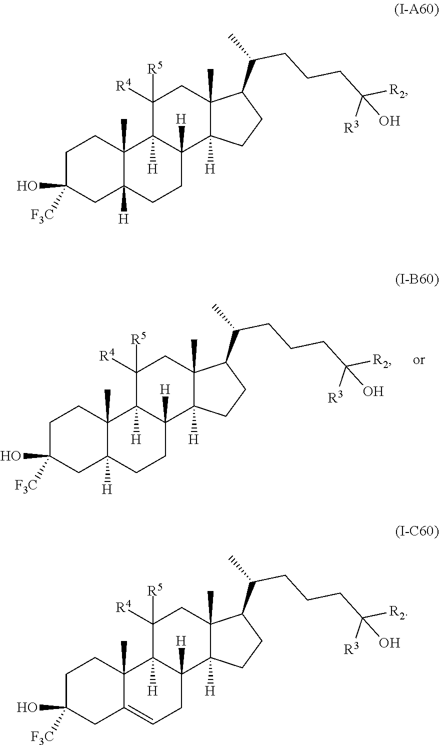

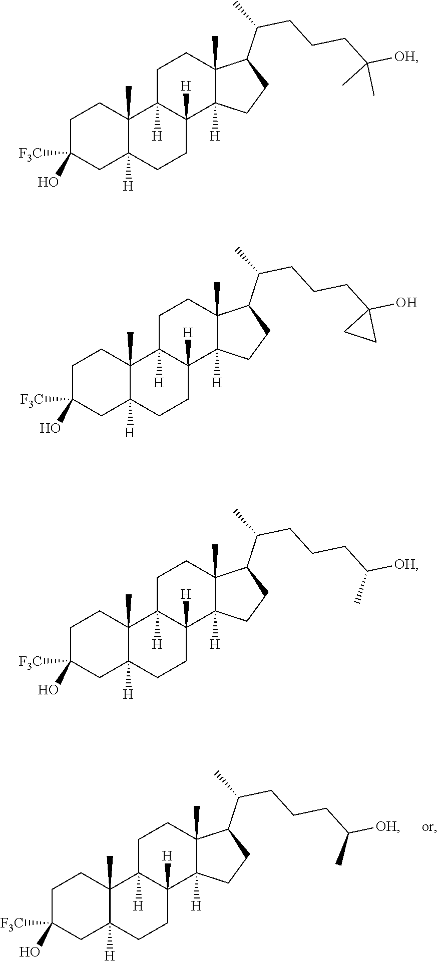

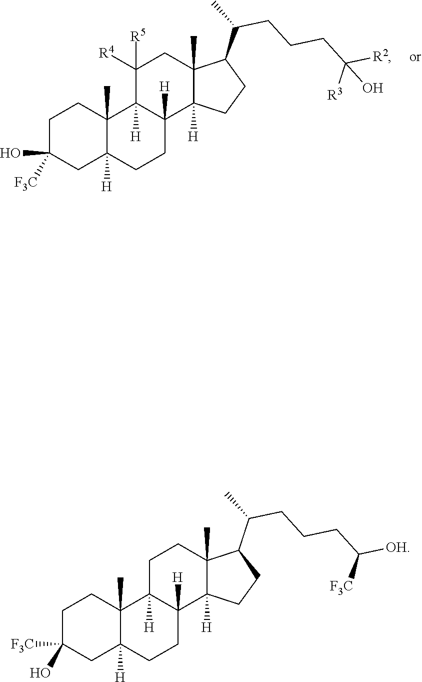

### Formula 208

In an embodiment, the NMDA receptor antagonist may be selected from one of the following compounds according to Formula 208. Such compounds are described in WO 2016/196513, published Dec. 8, 2016, corresponding to PCT/US2016/035098, filed May 31, 2016, and U.S. Pat. No. 10,584,127 issued Mar. 10, 2020, which are each incorporated by reference in their entirety herein. In the case of any conflict of terminology of Formula 208, these references incorporated by reference herein control.

In an embodiment, the NMDA receptor antagonist is a compound according to Formula 208:

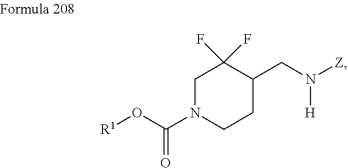

wherein R¹ is alkyl, cycloalkyi, (cycloalkyl)alkyl, heterocyclyl, (heterocyclyl)alkyl, aryl, (aryl)alkyl, heteroaryl or (heteroaryl)alkyl, wherein each of cycloalkyi, (cycloalkyl)alkyl, heterocyclyl, (heterocyclyl)alkyl, aryl, (aryl)alkyl, heteroaryl and (heteroaryl)alkyl is independently optionally substituted with 1 to 3 groups independently selected from —F, —Cl, C₁-C₄ alkyl, cyclopropyl, —C≡CH, —CFH2, —CF2H, —CF3, —CF2CH₃, —CH2CF3, C₁-C₄ alkoxy, —OCFH2, —OCF2H, —OCF3, —CN, —N(R2)(R3), —N02, C₁-C₄ alkylthio, C₁-C₄ alkylsulfonyl and —S(0)2CF3; wherein each instance of R² and R3 independently is —H or C₁-C₄ alkyl, or

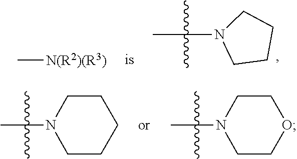

Z is 5- or 6-membered monocyclic or 9- or 10-membered bicyclic heteroaryl having ring carbon atoms, 1 nitrogen ring atom and 0-3 additional ring heteroatoms independently selected from N, O and S, which is optionally substituted with 1 or 2 Rx groups and optionally substituted with 1 Ra group, wherein each Rx is attached to a ring carbon atom and Ra is attached to a ring nitrogen atom; wherein: each instance of Rx independently is —F, —Cl, —CH₃, —CFH2, —CF2H, —CF3, —OH, —OCH₃, —OCF3 or —CN; and Ra is C1-C4 alkyl, C3-C4 cycloalkyl or —S( ))2-C1-C4 alkyl, including pharmaceutically acceptable salts thereof.

In further embodiments, the NMDA receptor antagonist is a compound selected from:

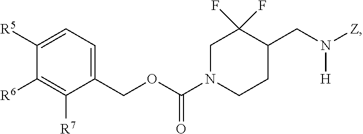

wherein R5, R6 and R7 independently are —H, —F, —Cl, C C alkyl, cyclopropyl, —C≡CH, —CFH2, —CF2H, —CF3, —CF2CH3, —CH2CF3, C1-C4 alkoxy, —OCFH2, —OCF2H, —OCF3, —CN, —N(R2) (R3), —NO2, C C4 alkylthio, C1-C4 alkylsulfonyl or —S(O)₂CF3; wherein each instance of R2 and R3 independently is —H or C1-C4 alkyl, or C1-C4 alkyl, or

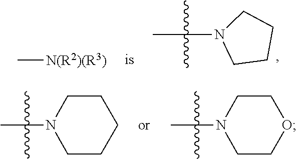

including pharmaceutically acceptable salts thereof.

In further embodiments, the NMDA receptor antagonist is a compound selected from:

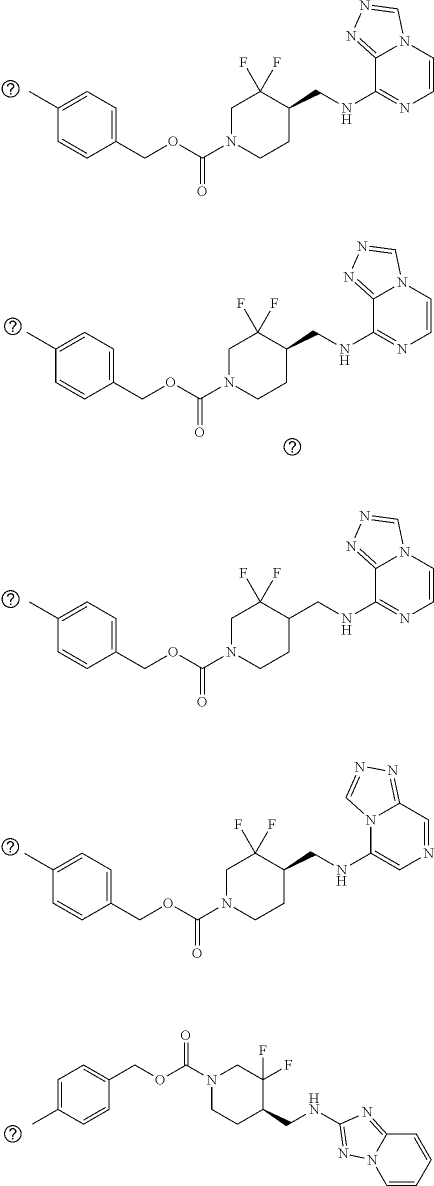 [SMILES: Cc1ccc(COC(=O)N2CC[C@H](CNc3nccn4cnnc34)C(F)(F)C2)cc1 ; Cc1ccc(COC(=O)N2CC[C@H](CNc3nccn4cnnc34)C(F)(F)C2)cc1 ; Cc1ccc(COC(=O)N2CCC(CNc3nccn4cnnc34)C(F)(F)C2)cc1 ; Cc1ccc(COC(=O)N2CC[C@H](CNc3cncc4nncn34)C(F)(F)C2)cc1 ; Cc1ccc(COC(=O)N2CC[C@H](CNc3nc4ccccn4n3)C(F)(F)C2)cc1]

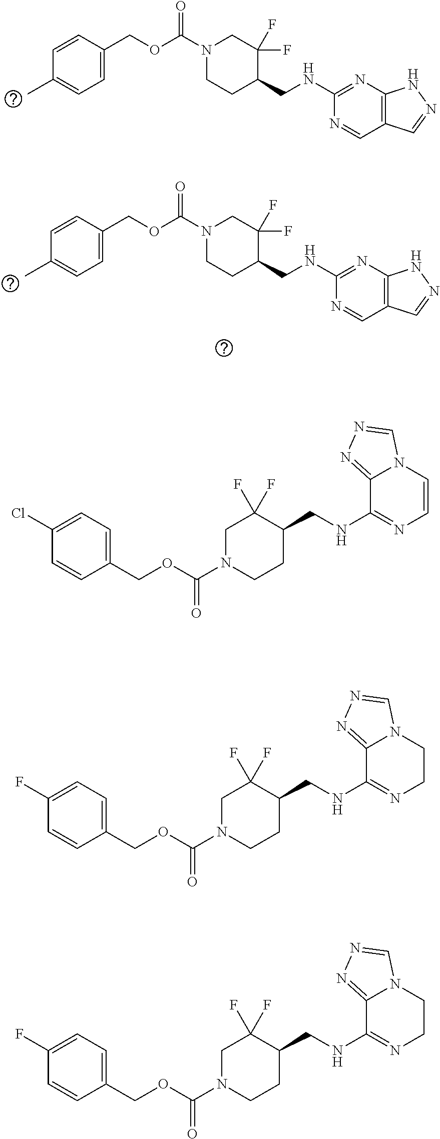 [SMILES: Cc1ccc(COC(=O)N2CC[C@H](CNc3ncc4cn[nH]c4n3)C(F)(F)C2)cc1 ; Cc1ccc(COC(=O)N2CC[C@H](CNc3ncc4cn[nH]c4n3)C(F)(F)C2)cc1 ; O=C(OCc1ccc(Cl)cc1)N1CC[C@H](CNc2nccn3cnnc23)C(F)(F)C1 ; O=C(OCc1ccc(F)cc1)N1CC[C@H](CNC2=NCCn3cnnc32)C(F)(F)C1 ; O=C(OCc1ccc(F)cc1)N1CC[C@H](CNC2=NCCn3cnnc32)C(F)(F)C1]

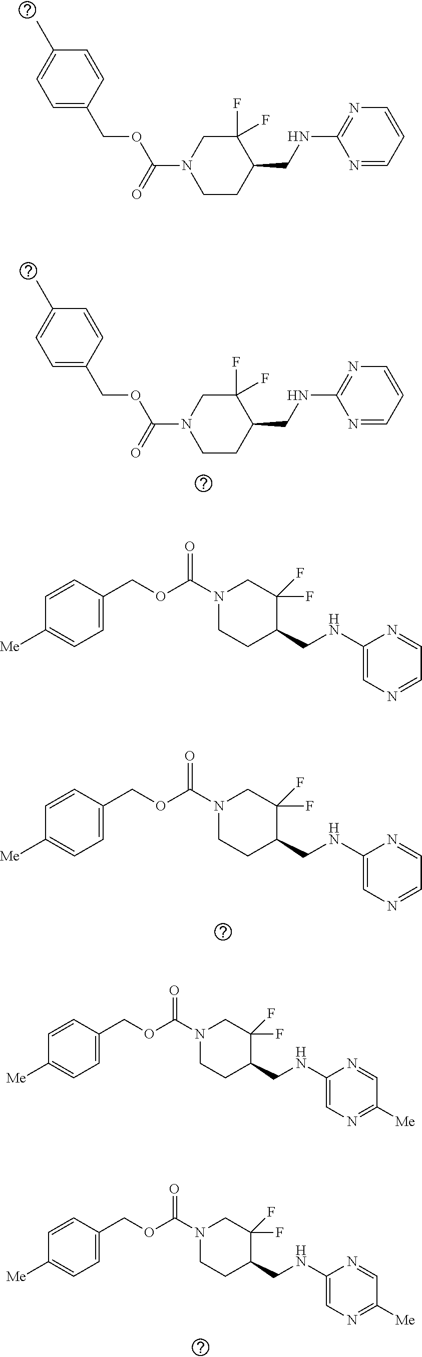 [SMILES: Cc1ccc(COC(=O)N2CC[C@H](CNc3ncccn3)C(F)(F)C2)cc1 ; Cc1ccc(COC(=O)N2CC[C@H](CNc3ncccn3)C(F)(F)C2)cc1 ; Cc1ccc(COC(=O)N2CC[C@H](CNc3cnccn3)C(F)(F)C2)cc1 ; Cc1ccc(COC(=O)N2CC[C@H](CNc3cnccn3)C(F)(F)C2)cc1 ; Cc1ccc(COC(=O)N2CC[C@H](CNc3cnc(C)cn3)C(F)(F)C2)cc1 ; Cc1ccc(COC(=O)N2CC[C@H](CNc3cnc(C)cn3)C(F)(F)C2)cc1]

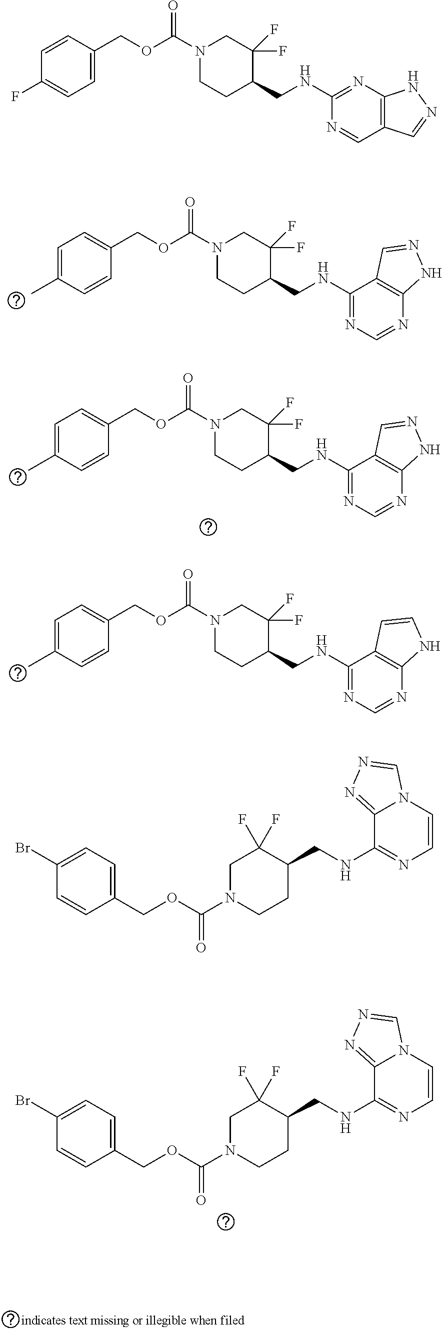 [SMILES: O=C(OCc1ccc(F)cc1)N1CC[C@H](CNc2ncc3cn[nH]c3n2)C(F)(F)C1 ; Cc1ccc(COC(=O)N2CC[C@H](CNc3ncnc4[nH]ncc34)C(F)(F)C2)cc1 ; Cc1ccc(COC(=O)N2CC[C@H](CNc3ncnc4[nH]ncc34)C(F)(F)C2)cc1 ; Cc1ccc(COC(=O)N2CC[C@H](CNc3ncnc4[nH]ccc34)C(F)(F)C2)cc1 ; O=C(OCc1ccc(Br)cc1)N1CC[C@H](CNc2nccn3cnnc23)C(F)(F)C1 ; O=C(OCc1ccc(Br)cc1)N1CC[C@H](CNc2nccn3cnnc23)C(F)(F)C1]

In further embodiments, the NMDA receptor antagonist is selected from:

| | |
|---|---|
| | (R) |
| 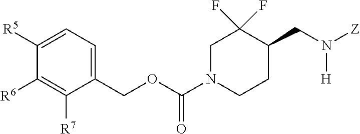 | |

| compound | Z | R⁵ | R⁶ | R⁷ |
|---|---|---|---|---|
| E1-22.1 | Z22 | H | H | H |
| E1-22.2 | Z22 | CH₃ | H | H |
| E1-22.3 | Z22 | Cl | H | H |
| E1-22.4 | Z22 | F | H | H |
| E1-22.5 | Z22 | CH₂CH₃ | H | H |
| E1-22.6 | Z22 | CF₂H | H | H |
| E1-22.7 | Z22 | CH₂F | H | H |
| E1-22.8 | Z22 | CF₃ | H | H |
| E1-22.9 | Z22 | CF₂CH₃ | H | H |
| E1-22.10 | Z22 | CH₂CF₃ | H | H |
| E1-22.11 | Z22 | cyclopropyl | H | H |
| E1-22.12 | Z22 | OCF₃ | H | H |
| E1-22.13 | Z22 | OCF₂H | H | H |
| E1-22.14 | Z22 | Cl | H | F |
| E1-22.15 | Z22 | CH₃ | H | F |
| E1-22.16 | Z22 | CH₃ | F | H |
| E1-22.17 | Z22 | Cl | F | H |
| E1-22.18 | Z22 | F | F | H |
| E1-22.19 | Z22 | F | H | F |
| E1-22.20 | Z22 | F | H | Cl |
| E1-22.21 | Z22 | F | H | CH₃ |
| E1-22.22 | Z22 | Cl | H | CH₃ |
| E1-22.23 | Z22 | SCH₃ | H | H |
| E1-22.24 | Z22 | SO₂CH₃ | H | H |
| E1-22.25 | Z22 | ethynyl | H | H |

In further embodiments, the NMDA receptor antagonist is selected from:

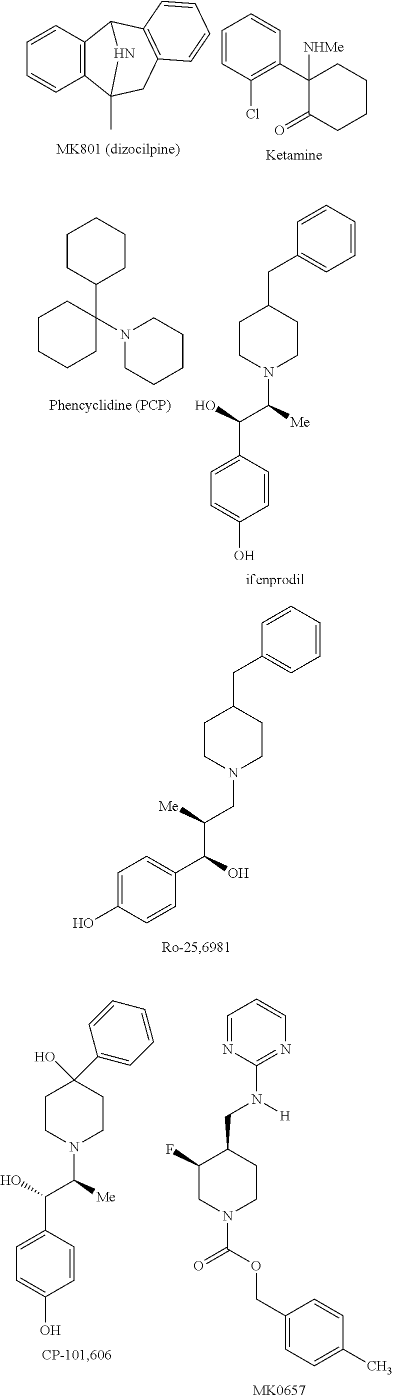

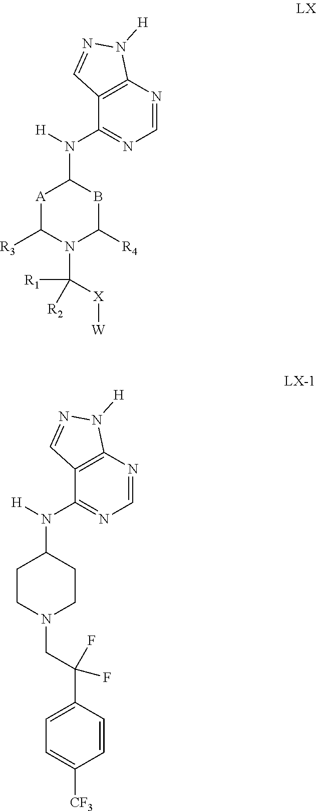

### Formula 209

In an embodiment, the NMDA receptor antagonist may be selected from one of the following compounds according to Formula 209. Such compounds are described in International application publication no. WO2010/088414, published Aug. 5, 2010, corresponding to PCT/US2010/022439, filed Jan. 28, 2010, International application publication no. WO 2010/088408, published Aug. 5, 2010, corresponding to PCT/US2010/022432, filed Jan. 28, 2010, and U.S. Pat. No. 8,822,462, issued Sep. 2, 2014, which are each incorporated by reference in their entirety herein. In the case of any conflict of terminology of Formula 209, these references incorporated by reference herein control.

In an embodiment, the NMDA receptor antagonist is a compound according to Formula 209a-209b:

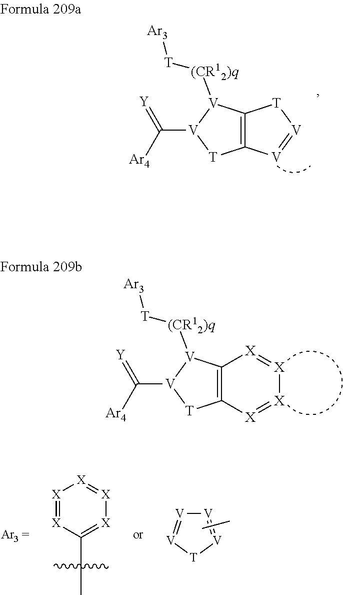

wherein:

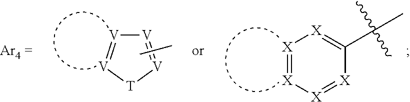

X is, independently, N or C bonded to H or a substituent, J, with the proviso that no more than three of X are N;
Y is independently selected from O, S, NR1, CH2, and CR2;
R1 and R2 are, independently, selected from H, alkyl, substituted alkyl, alkenyl, substituted alkenyl, aryl, substituted aryl, heteroaryl, substituted heteroaryl, and hydroxy, and, when R1 is attached to a carbon atom, it can be halo or cyano,
T is, independently, CHR1, CR2, O, S, or NR1,
V is, independently, N, or C bonded to H or a substituent J,
J is a non-hydrogen substituent selected from the group consisting of halo (—F, —Cl, —Br, —I), nitro, amino (NR1R2), OR1, SR1, —R1, —CF3, —CN, —C2R1, —SO₂CH₃, —CC═O)NR1R2-NR1CC═O)R1, —CC═O)R1, —CC═O)OR1, —(CHz)9OR1, —OCC═O)R1, —OCC═O)NR1R2, —NR1CC═Y)—NR1R2, —NR1CC═Y)—OH, —NR1CC═Y)—SH, sulfonyl, sulfinyl, phosphoryl, and azo, and q is 0-5, wherein the aryl ring moiety Ar⁴ can optionally be replaced with a saturated ring, and pharmaceutically acceptable salt, esters, prodrugs, or derivatives thereof.

In further embodiments, the NMDA receptor antagonist is a compound according to Formula 209c:

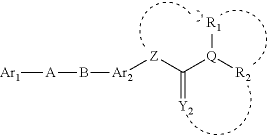

wherein A-B is a linker moiety selected from:

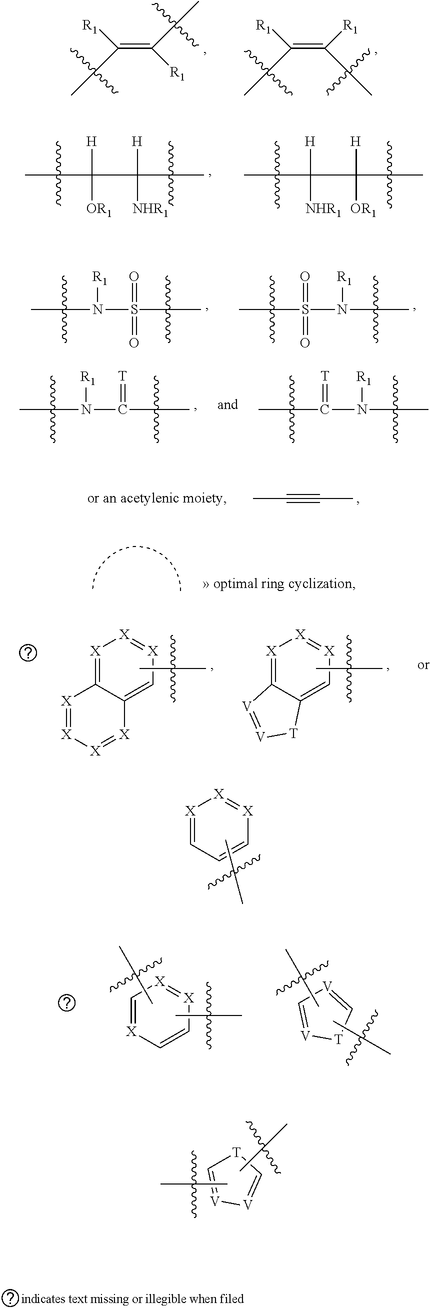

X is, independently, N, or C bonded to H or a substituent, J, with the proviso that no more than three of X are N;
Y1 and Y2 are, independently, selected from O, S, NR1, CH2, and CRX¹₂;
R1 and R2 are independently selected from H, alkyl, substituted alkyl, alkenyl, substituted alkenyl, aryl, substituted aryl, heteroaryl, substituted heteroaryl, and hydroxy,
R1 and R2 can optionally join to form a C3-10 heterocyclic moiety, which heterocyclic moiety can optionally include a second heteroatom selected from O, S, and N,
R2 is absent when Q is O or S,
Z is, independently, (CH2)ₙ, CHR, CR2, O, S, or NR1,
T is, independently, CHR, C(R1)2, O, S, or NR1,
Q is independently selected from CH, C-halo, or N, or O or S if R² is absent,
V is, independently, N or C bonded to H or a substituent J,
J is a non-hydrogen substituent selected from the group consisting of halo (—F, —Cl, —Br, —I), nitro, amino (NR1R2), OR1, SR1, —R1, —CF3, —CN, —C2R1, —SO2CH3, -Q=O)NR1R2-NR1CX═O)R1, —O═O)R1, —CC═O)OR1, —(CH2), OR1, —OC(═O)R1, —OQ=O)NR1R2, —NR1CC═Y)—NR1R2, —NR1CC═Y)—OH, —NR1CC═Y)—SH, sulfonyl, sulfinyl, —SO2NHR1, —NHSO2R, phosphoryl, and azo, and pharmaceutically-acceptable salts, prodrugs, and esters thereof.

In further embodiments, the NMDA receptor antagonist is a compound selected from:

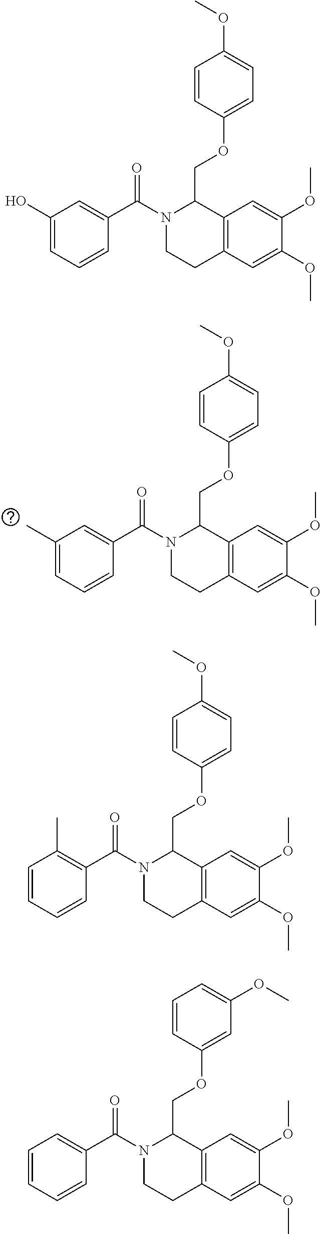

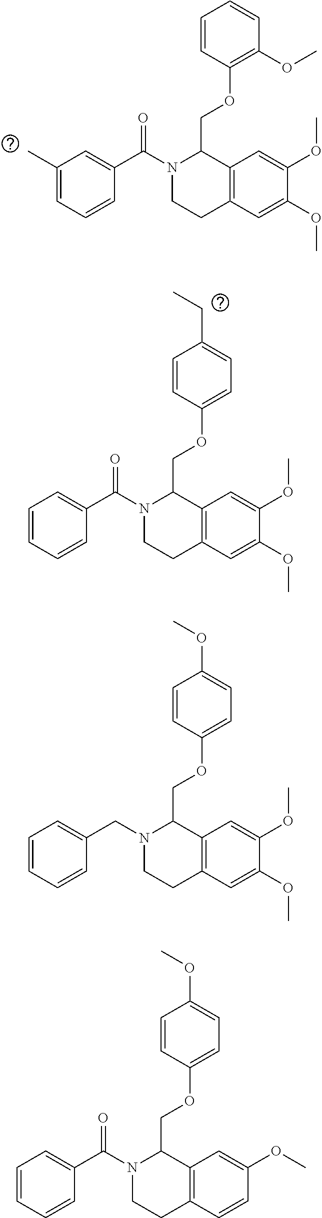

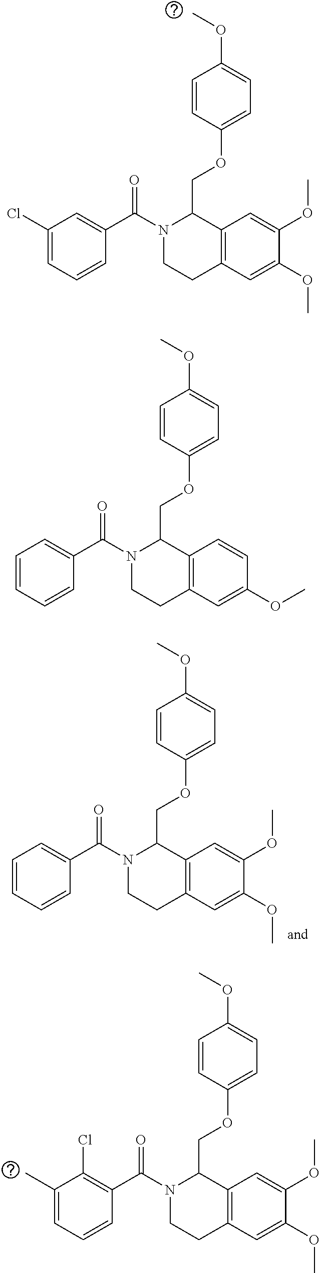

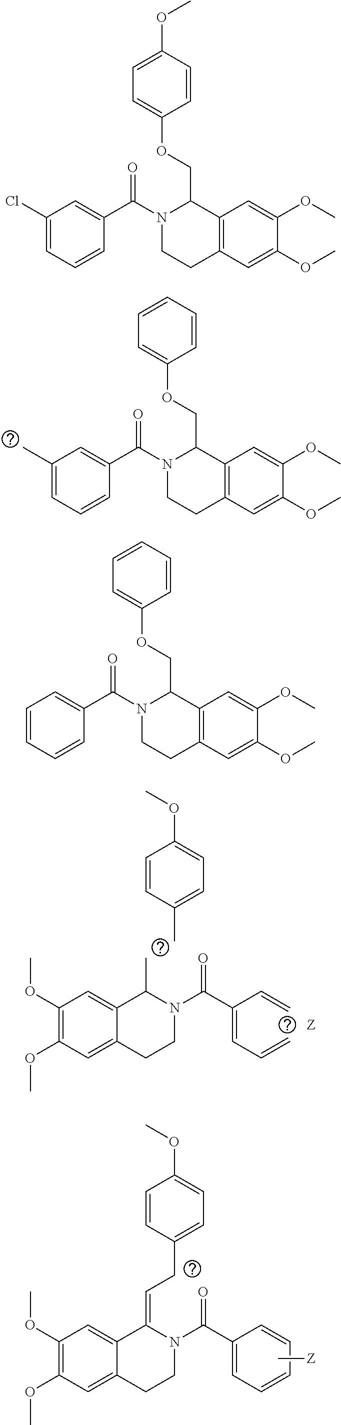

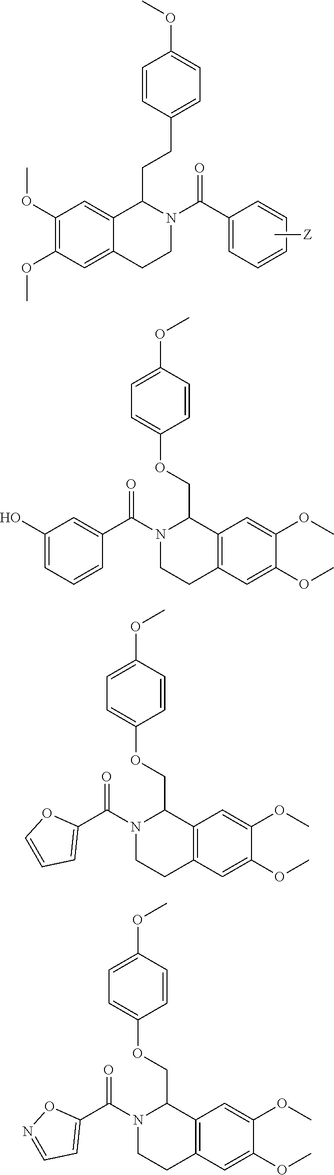

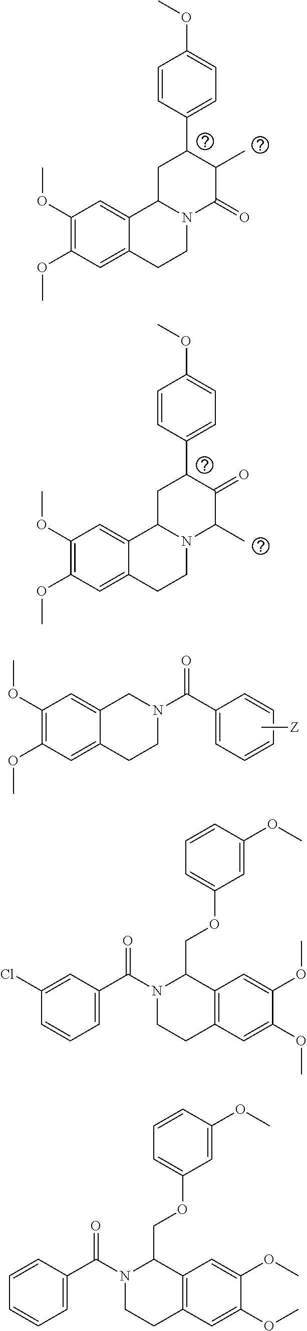

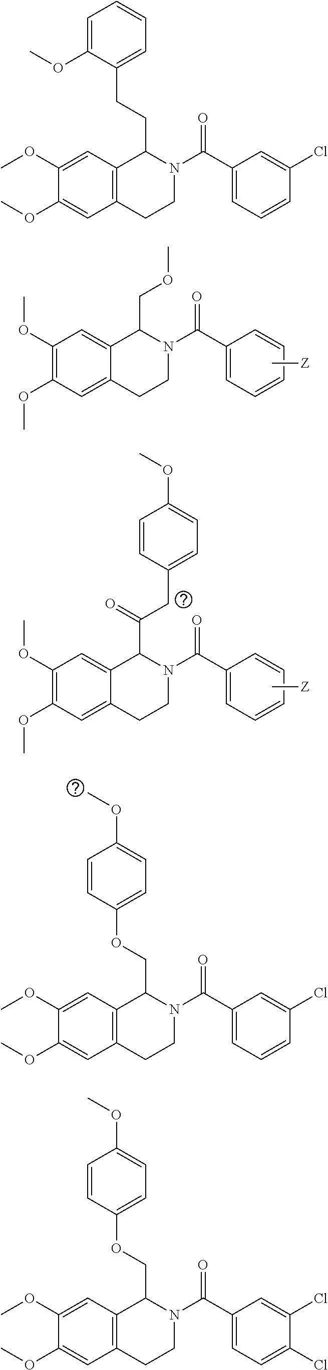

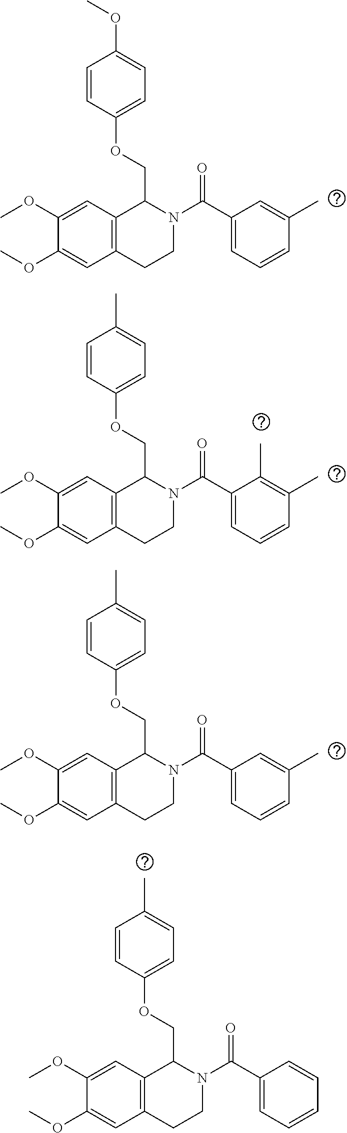

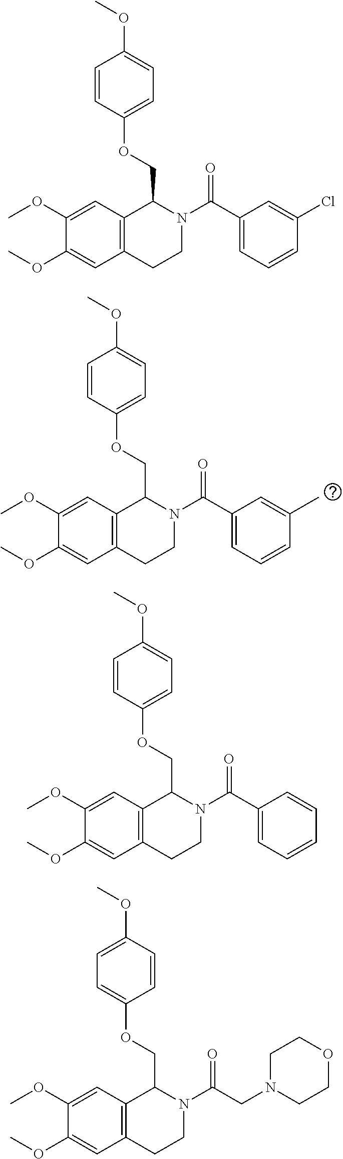

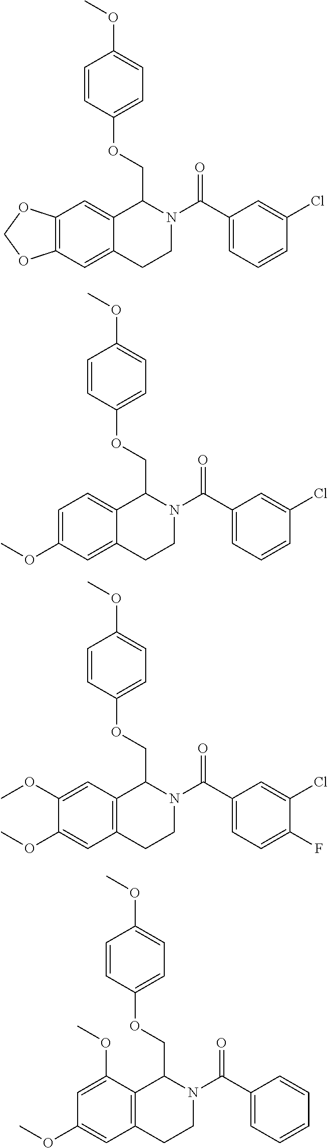

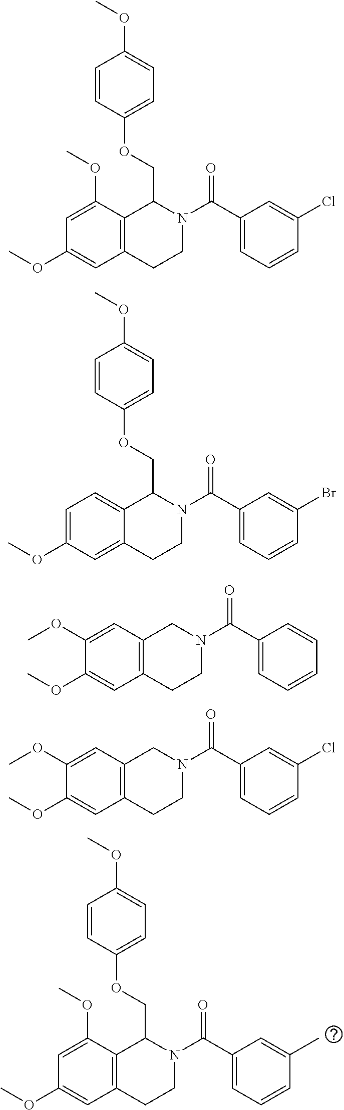

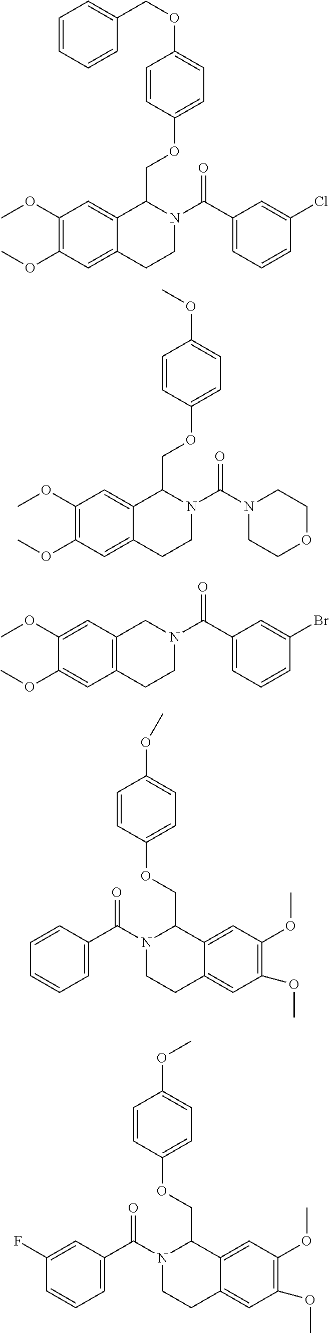

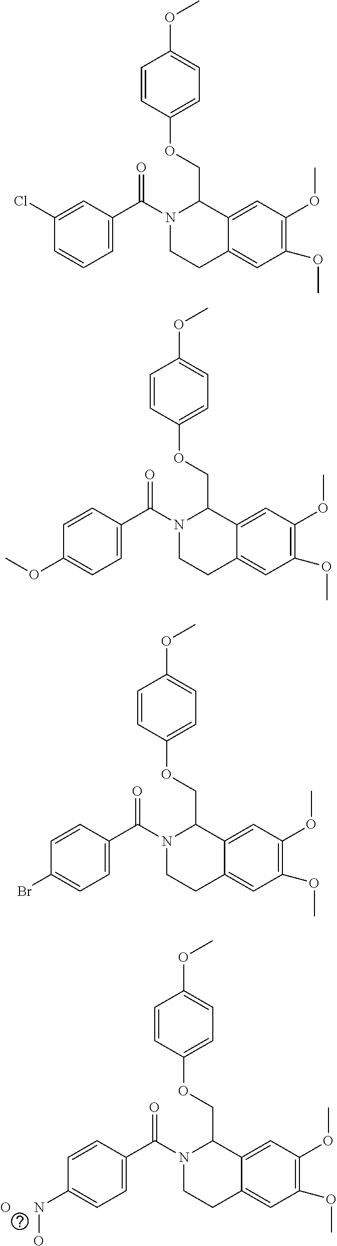

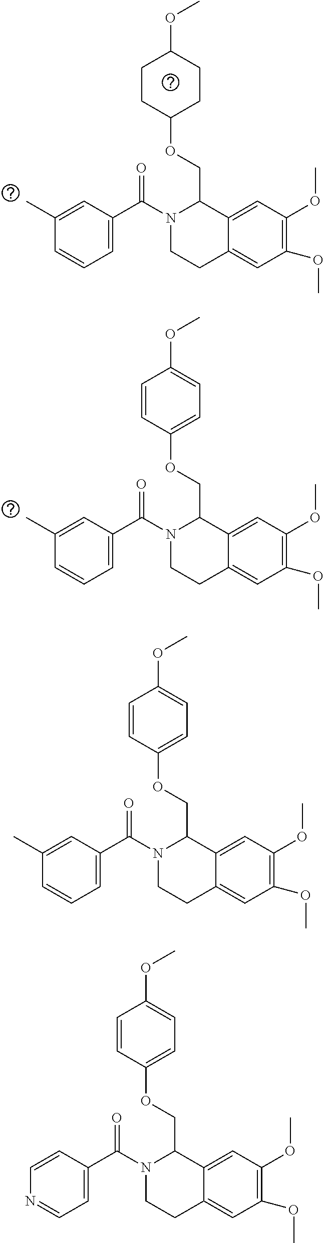

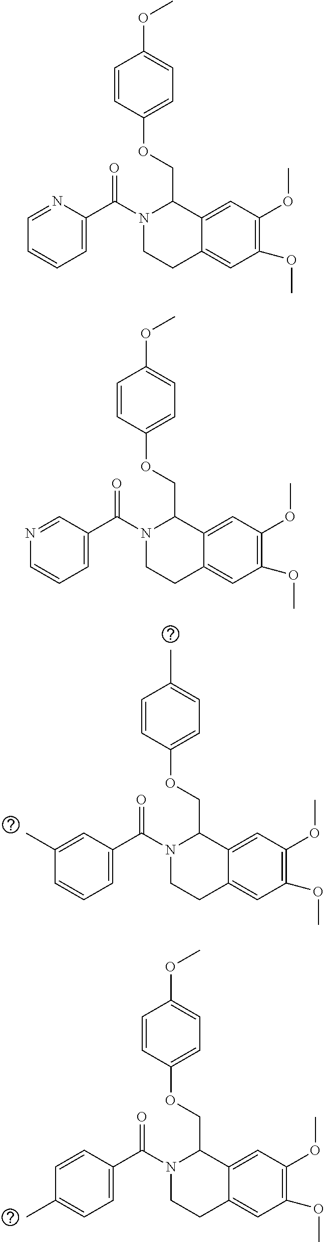

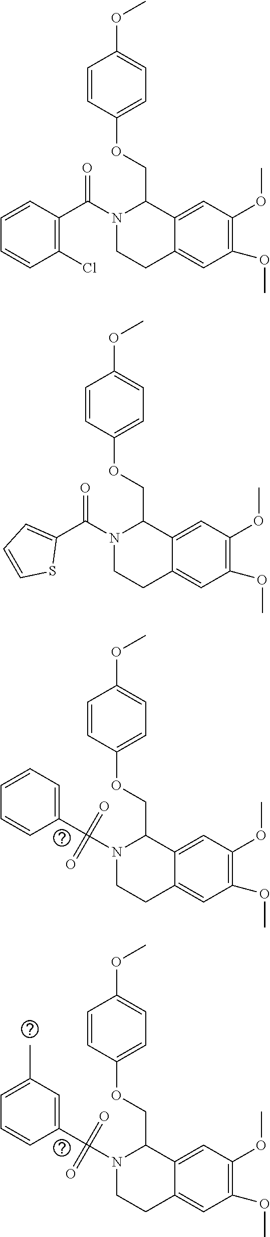

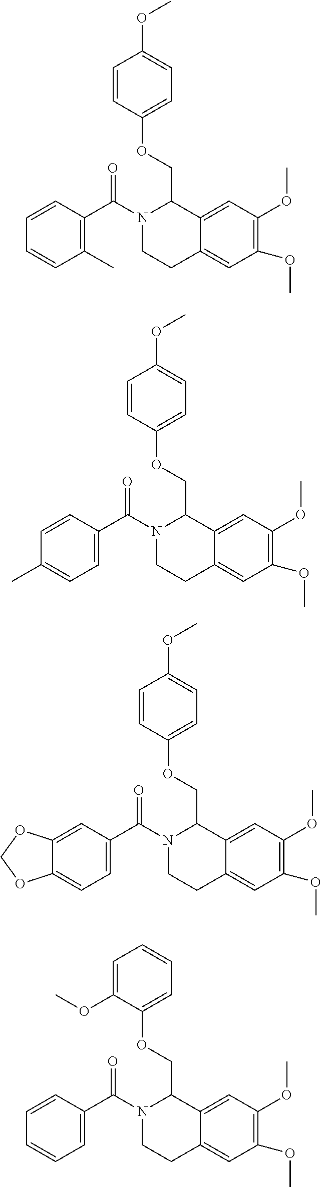

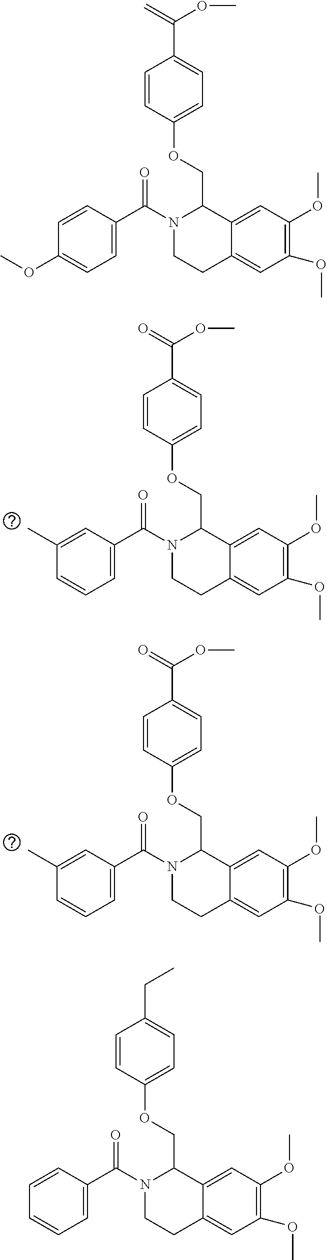

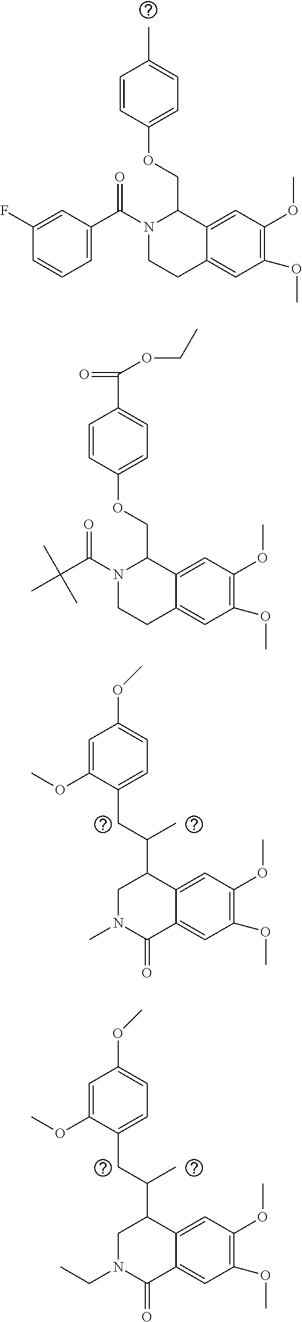

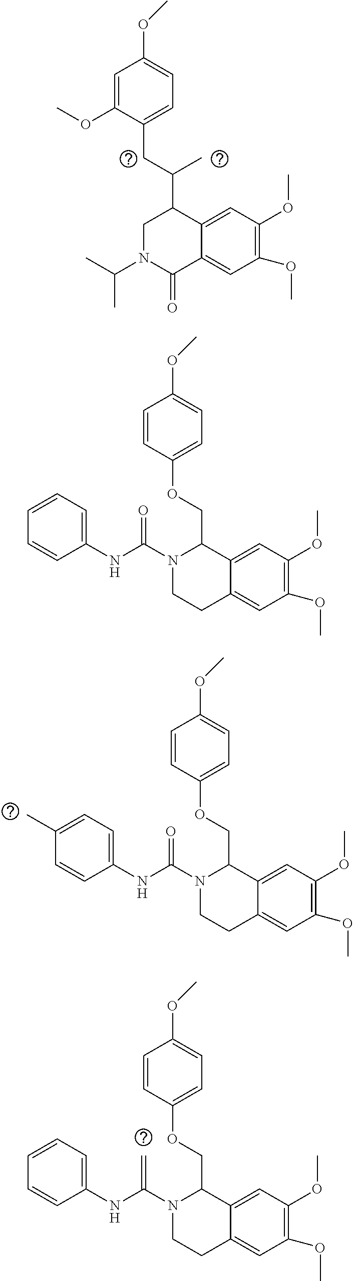

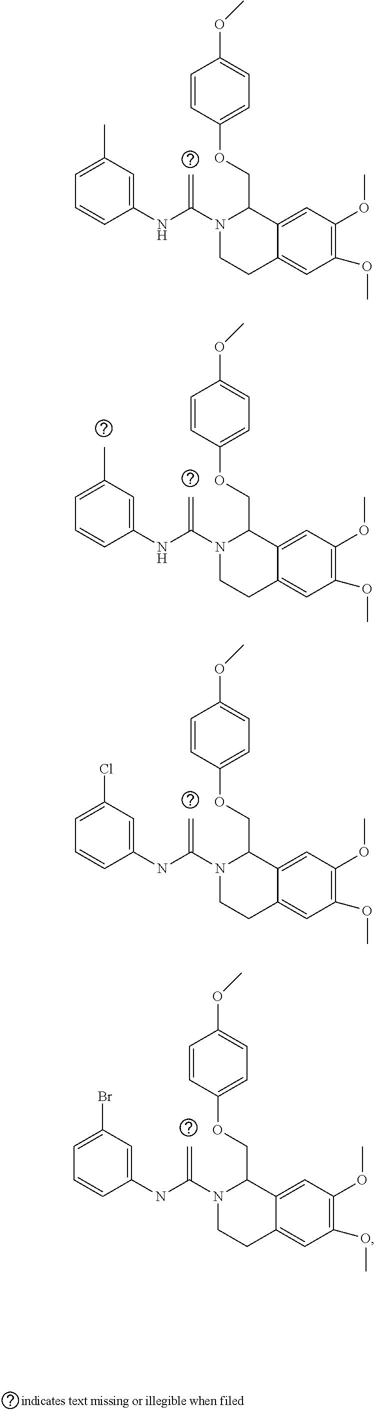 [SMILES: C=C(Nc1cccc(C)c1)N1CCc2cc(OC)c(OC)cc2C1COc1ccc(OC)cc1 ; C=C(Nc1cccc(C)c1)N1CCc2cc(OC)c(OC)cc2C1COc1ccc(OC)cc1 ; C=C(Nc1cccc(Cl)c1)N1CCc2cc(OC)c(OC)cc2C1COc1ccc(OC)cc1 ; C=C(Nc1cccc(Br)c1)N1CCc2cc(OC)c(OC)cc2C1COc1ccc(OC)cc1]

and pharmaceutically acceptable salts, esters, prodrugs, or derivatives thereof.

### D

In additional embodiments, the NMDA receptor antagonist has a structure according to Formula I or J:

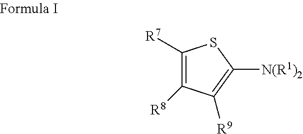

wherein R1 is as defined above in claim 1, R7 is selected from the group consisting of H, —Ci_6 alkyl, —Ci_6 substituted alkyl, —C6-io aryl, —Ce io substituted aryl, —Ce io heteroaryl, —Ce io substituted heteroaryl, —C(O)-alkyl, —C(O)-substituted alkyl, —C(O)-aryl, —C(O)-substituted aryl, —C(O)-arylalkyl, C(O)-substituted arylalkyl, —C(O)-alkylaryl, —C(O)-substituted alkylaryl-CN, N3, NO2, —OH, —NH2, —SH, —OR1, —NHR1, —N(RIK-SR1, —OC(O)R1, NHC(O)R1, —SC(O)R1, —OC(O)OR1, —NHC(O)R1, —CH2OH, —CH2CN, —CH2N3, —CO2R1, —CON(R1), —C(O)-alkyl, —C(O)-substituted alkyl, —C(O)-aryl, —C(O)-substituted aryl, —C(O)-heteroaryl, and —C(O)— substituted heteroaryl,

R8& R9 are, individually, H, OH, —NH2, —SH, —OR1, —NHR1, —N(RIK-SR1, —OC(O)R1, NHC(O)R1, —C1-6 alkyl, —C1-6 substituted alkyl, —C1-6 aryl, —C1-6 substituted aryl, —C6-10 heteroaryl, —C6-10 substituted heteroaryl,

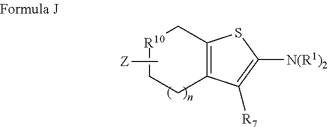

wherein R1 is as defined above,
R1 is as defined above,
R7 is selected from the group consisting of H, —C1-6 alkyl, —C1-6 substituted alkyl, —C6-10 aryl, C6-10 substituted aryl, —C6-10 heteroaryl, —C6-10 substituted heteroaryl, —C(O)-alkyl, —C(O)-substituted alkyl, —C(O)-aryl, —C(O)-substituted aryl, —C(O)-arylalkyl, C(O)-substituted arylalkyl, —C(O)-alkylaryl, —C(O)-substituted alkylaryl-CN, N3, NO2, —OH, —NH2, —SH, —OR1, —NHR1, —N(R1)2-SR1, —OC(O)R1, NHC(O)R1, —SC(O)R1, —OC(O)OR1, —NHC(O)R1, —CH2OH, —CH2CN, —CH2N3, —CO2R1, —CONtR1)\hat{ }-C(O)-alkyl, —C(O)-substituted alkyl, —C(O)-aryl, —C(O)-substituted aryl, —C(O)-heteroaryl, and —C(O)— substituted heteroaryl,
R10 is —CH2-, —S—, -0-, —NHR1-, —N(R:)2-, —C(O)—, —C(S)—, —C(NR1V,
N is 0-4, and
Z is a substituent as defined in claim 1, and pharmaceutically acceptable salts, esters, prodrugs, or derivatives thereof.

In further embodiments, the NMDA receptor antagonist is a compound according to:

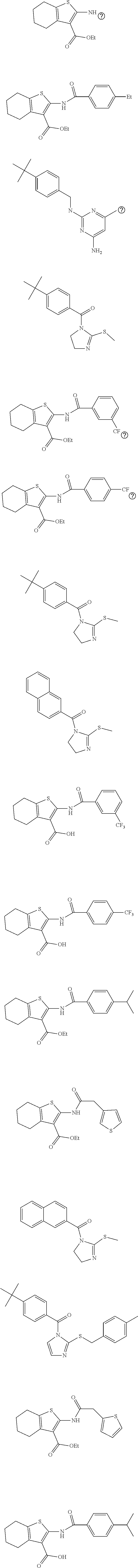

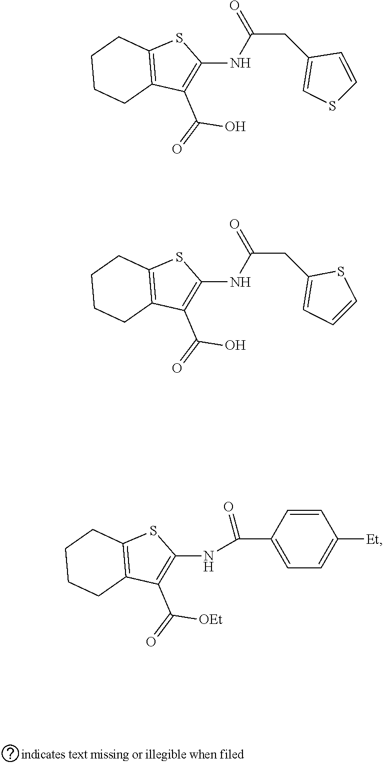 [SMILES: O=C(Cc1ccsc1)Nc1sc2c(c1C(=O)O)CCCC2 ; O=C(Cc1cccs1)Nc1sc2c(c1C(=O)O)CCCC2 ; CCOC(=O)c1c(NC(=O)c2ccc(CC)cc2)sc2c1CCCC2]

including pharmaceutically acceptable salts, esters, prodrugs, and derivatives thereof.

In further embodiments, the NMDA receptor antagonist is a compound according to:

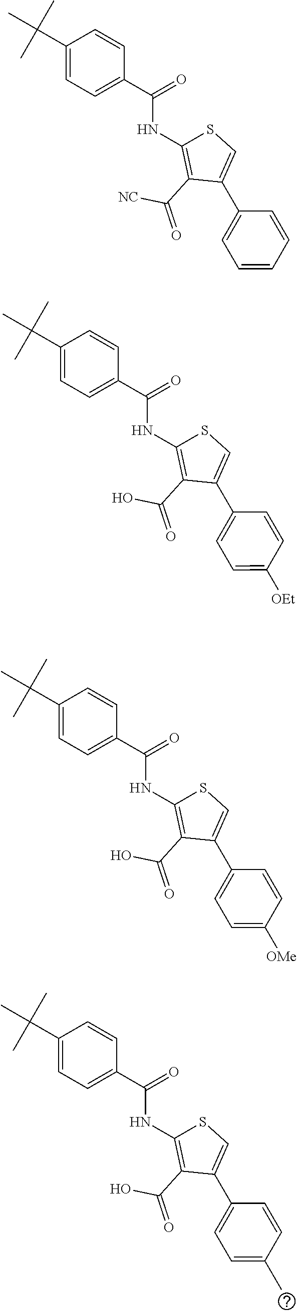 [SMILES: CC(C)(C)c1ccc(C(=O)Nc2scc(-c3ccccc3)c2C(=O)C#N)cc1 ; CCOc1ccc(-c2csc(NC(=O)c3ccc(C(C)(C)C)cc3)c2C(=O)O)cc1 ; COc1ccc(-c2csc(NC(=O)c3ccc(C(C)(C)C)cc3)c2C(=O)O)cc1 ; Cc1ccc(-c2csc(NC(=O)c3ccc(C(C)(C)C)cc3)c2C(=O)O)cc1]

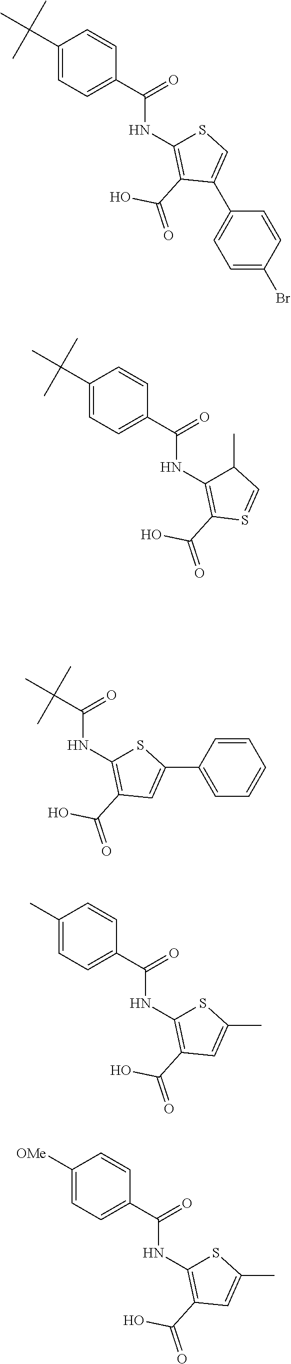

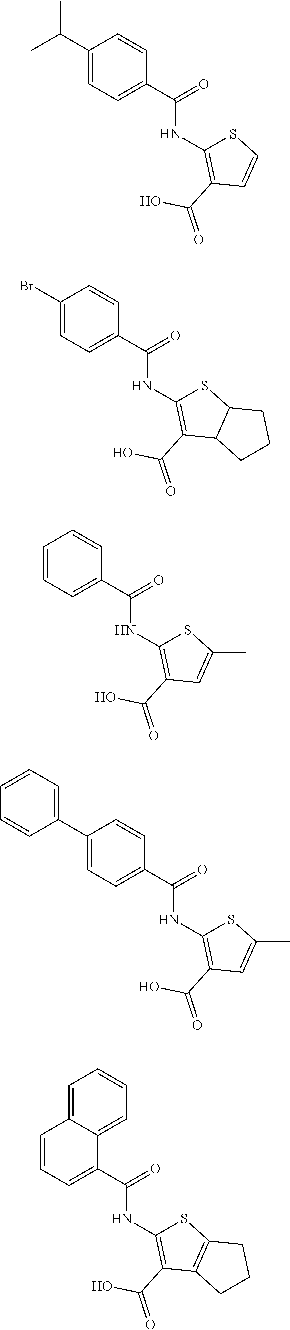 [SMILES: CC(C)c1ccc(C(=O)Nc2sccc2C(=O)O)cc1 ; O=C(O)C1=C(NC(=O)c2ccc(Br)cc2)SC2CCCC12 ; Cc1cc(C(=O)O)c(NC(=O)c2ccccc2)s1 ; Cc1cc(C(=O)O)c(NC(=O)c2ccc(-c3ccccc3)cc2)s1 ; O=C(O)c1c(NC(=O)c2cccc3ccccc23)sc2c1CCC2]

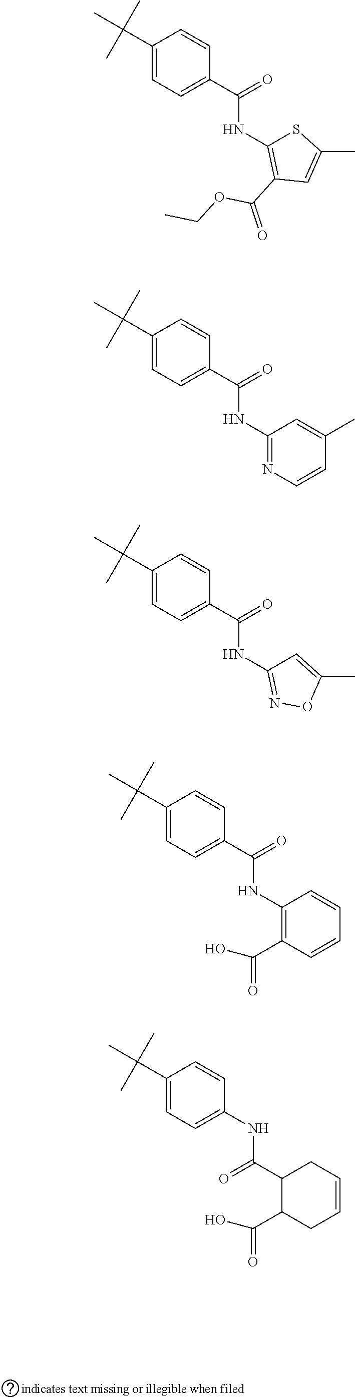

and pharmaceutically acceptable salts, esters, prodrugs, or derivatives thereof.

In further embodiments, the NMDA receptor antagonist is a compound according to:

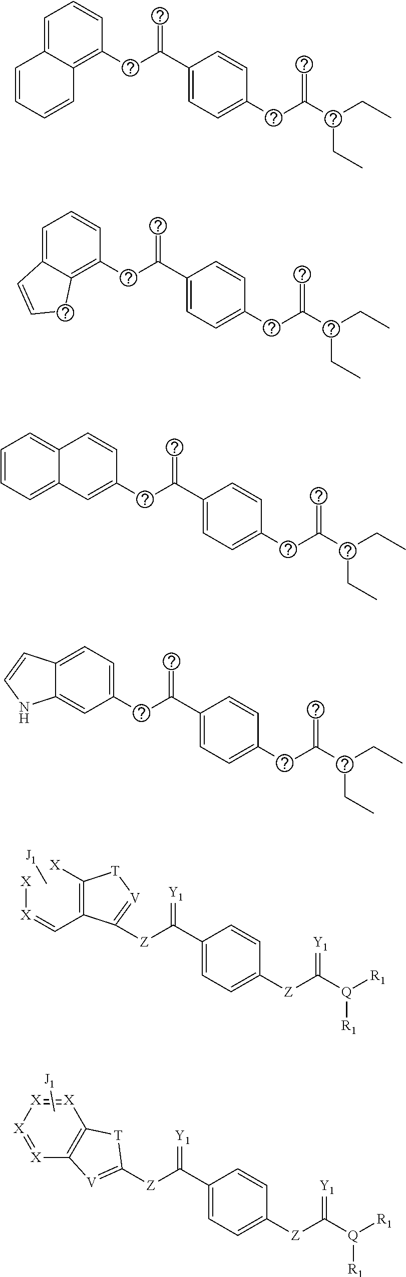

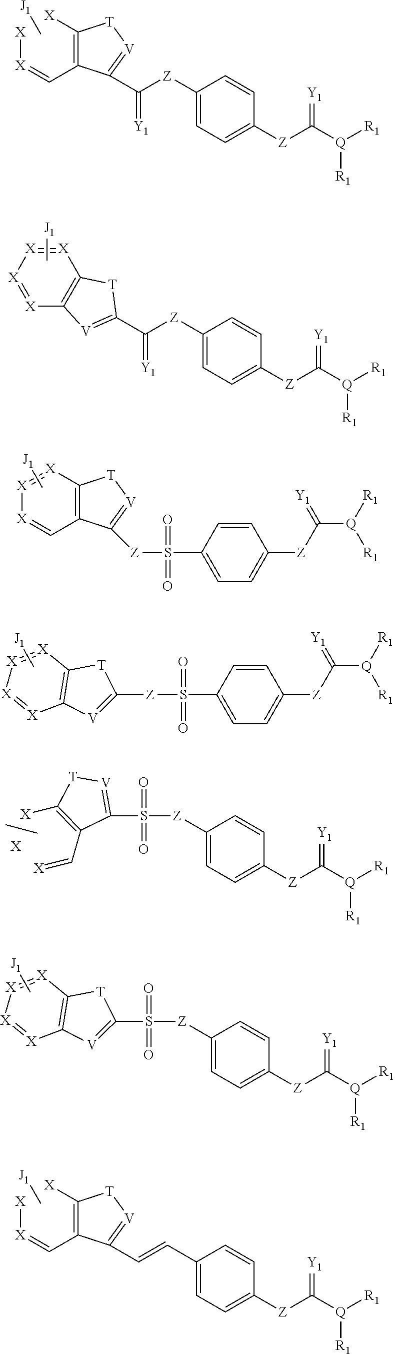

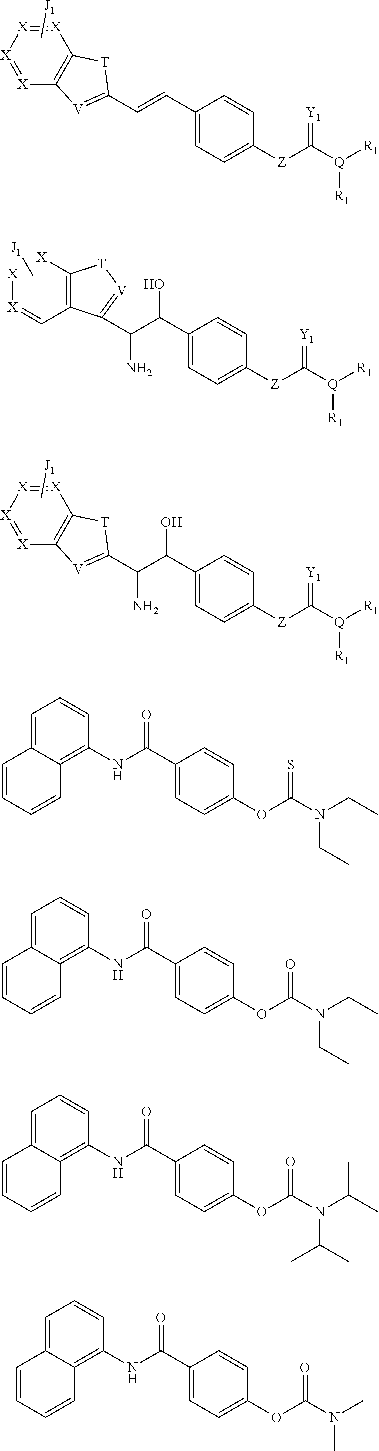

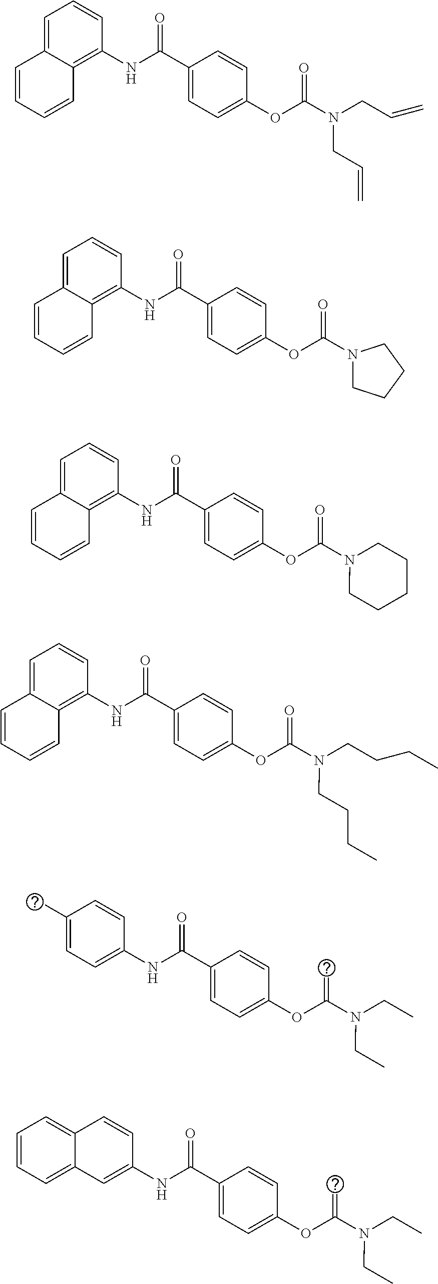

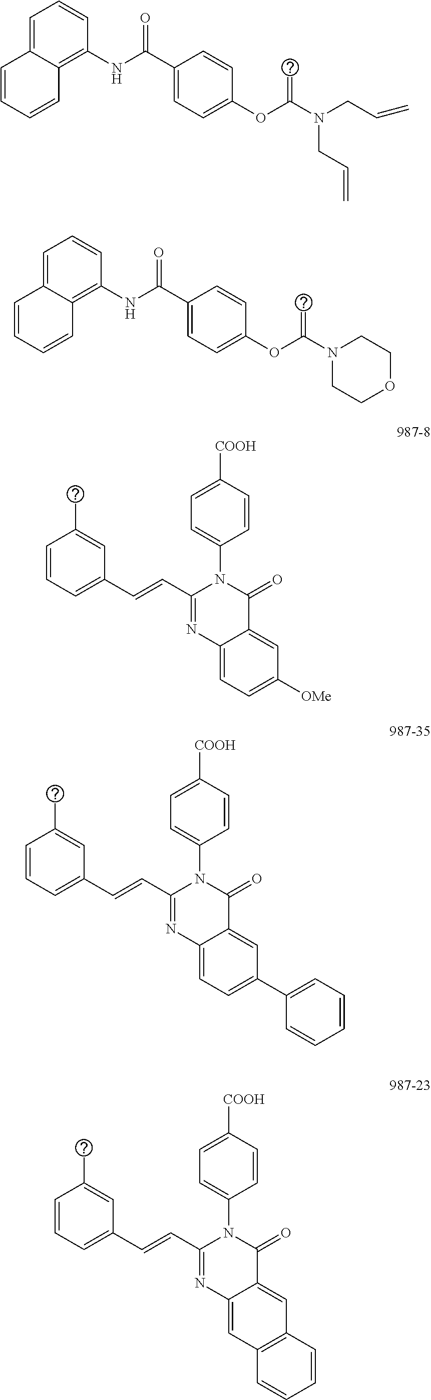 [SMILES: C=CCN(CC=C)C(=O)Oc1ccc(C(=O)Nc2cccc3ccccc23)cc1 ; O=C(Nc1cccc2ccccc12)c1ccc(OC(=O)N2CCOCC2)cc1 ; COc1ccc2nc(/C=C/c3cccc(C)c3)n(-c3ccc(C(=O)O)cc3)c(=O)c2c1 ; Cc1cccc(/C=C/c2nc3ccc(-c4ccccc4)cc3c(=O)n2-c2ccc(C(=O)O)cc2)c1 ; Cc1cccc(/C=C/c2nc3cc4ccccc4cc3c(=O)n2-c2ccc(C(=O)O)cc2)c1]

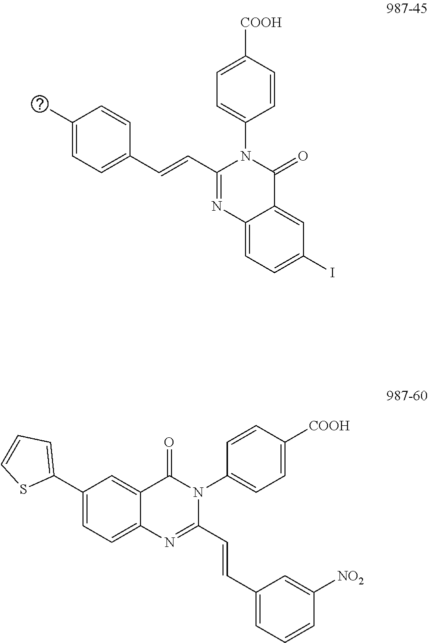 [SMILES: Cc1ccc(/C=C/c2nc3ccc(I)cc3c(=O)n2-c2ccc(C(=O)O)cc2)cc1 ; O=C(O)c1ccc(-n2c(/C=C/c3cccc([N+](=O)[O-])c3)nc3ccc(-c4cccs4)cc3c2=O)cc1]

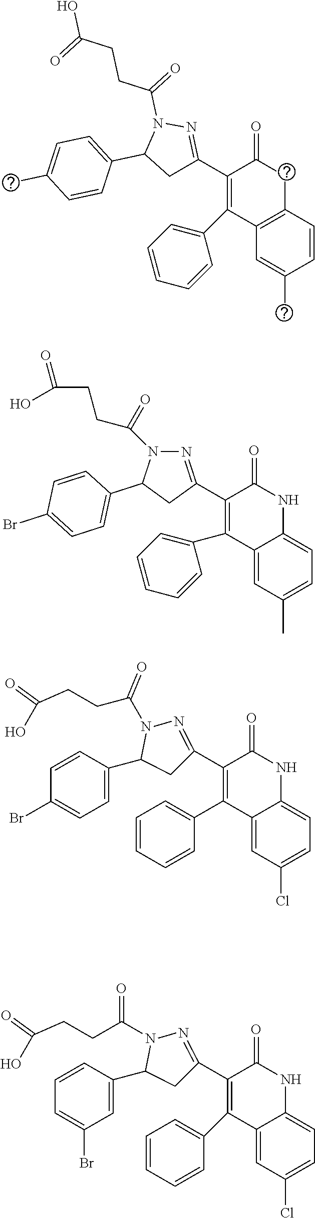 [SMILES: Cc1ccc(C2CC(C3=C(c4ccccc4)c4cc(C)ccc4CC3=O)=NN2C(=O)CCC(=O)O)cc1 ; Cc1ccc2[nH]c(=O)c(C3=NN(C(=O)CCC(=O)O)C(c4ccc(Br)cc4)C3)c(-c3ccccc3)c2c1 ; O=C(O)CCC(=O)N1N=C(c2c(-c3ccccc3)c3cc(Cl)ccc3[nH]c2=O)CC1c1ccc(Br)cc1 ; O=C(O)CCC(=O)N1N=C(c2c(-c3ccccc3)c3cc(Cl)ccc3[nH]c2=O)CC1c1cccc(Br)c1]

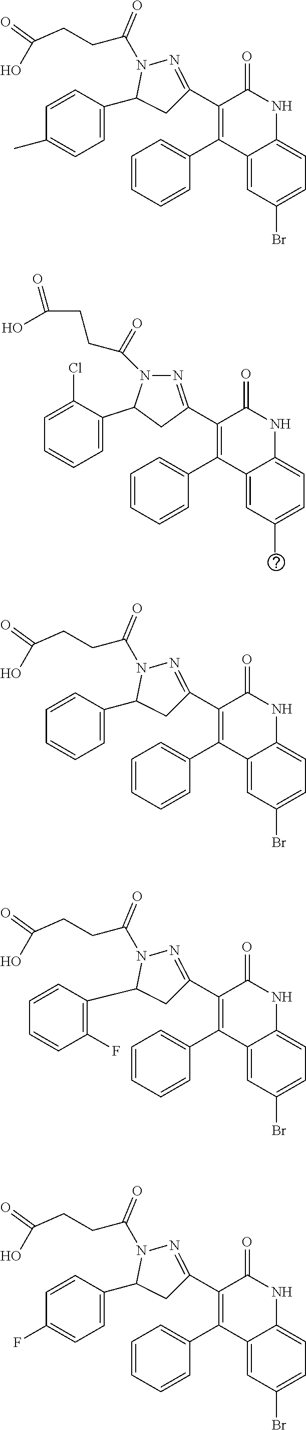 [SMILES: Cc1ccc(C2CC(c3c(-c4ccccc4)c4cc(Br)ccc4[nH]c3=O)=NN2C(=O)CCC(=O)O)cc1 ; Cc1ccc2[nH]c(=O)c(C3=NN(C(=O)CCC(=O)O)C(c4ccccc4Cl)C3)c(-c3ccccc3)c2c1 ; O=C(O)CCC(=O)N1N=C(c2c(-c3ccccc3)c3cc(Br)ccc3[nH]c2=O)CC1c1ccccc1 ; O=C(O)CCC(=O)N1N=C(c2c(-c3ccccc3)c3cc(Br)ccc3[nH]c2=O)CC1c1ccccc1F ; O=C(O)CCC(=O)N1N=C(c2c(-c3ccccc3)c3cc(Br)ccc3[nH]c2=O)CC1c1ccc(F)cc1]

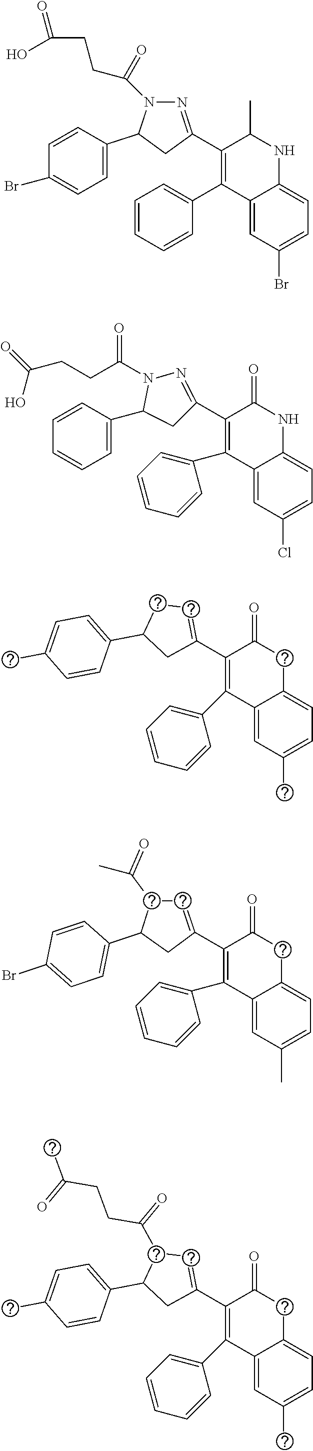 [SMILES: CC1Nc2ccc(Br)cc2C(c2ccccc2)=C1C1=NN(C(=O)CCC(=O)O)C(c2ccc(Br)cc2)C1 ; O=C(O)CCC(=O)N1N=C(c2c(-c3ccccc3)c3cc(Cl)ccc3[nH]c2=O)CC1c1ccccc1 ; Cc1ccc(C2CC=C(C3=C(c4ccccc4)c4cc(C)ccc4CC3=O)C2)cc1 ; CC(=O)C1C=C(C2=C(c3ccccc3)c3cc(C)ccc3CC2=O)CC1c1ccc(Br)cc1 ; CC(=O)CCC(=O)C1C=C(C2=C(c3ccccc3)c3cc(C)ccc3CC2=O)CC1c1ccc(C)cc1]

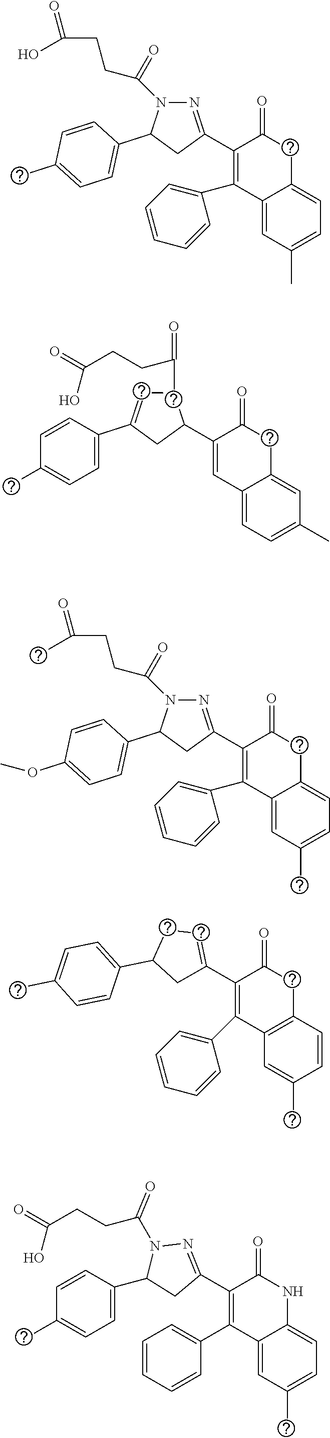

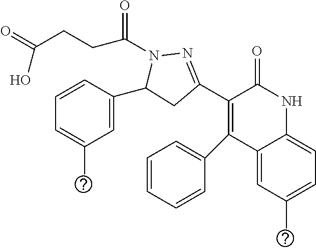 [SMILES: Cc1cccc(C2CC(c3c(-c4ccccc4)c4cc(C)ccc4[nH]c3=O)=NN2C(=O)CCC(=O)O)c1]

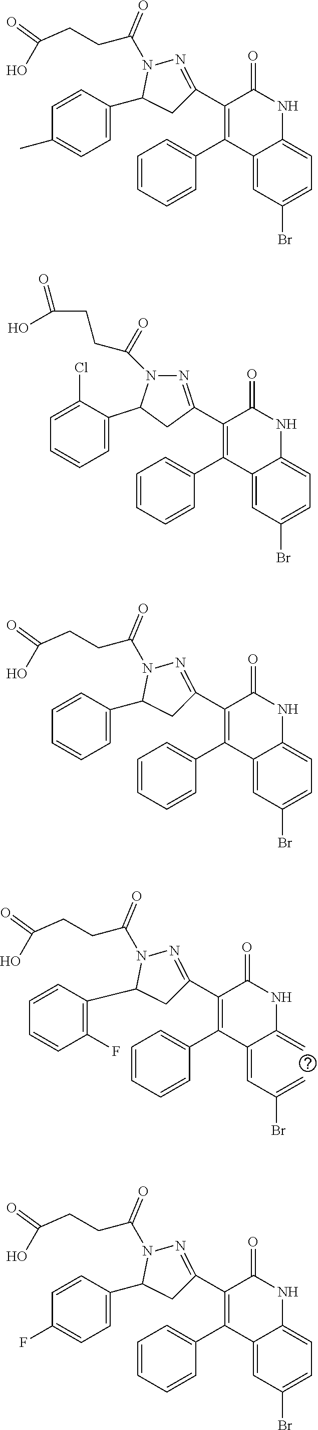 [SMILES: Cc1ccc(C2CC(c3c(-c4ccccc4)c4cc(Br)ccc4[nH]c3=O)=NN2C(=O)CCC(=O)O)cc1 ; O=C(O)CCC(=O)N1N=C(c2c(-c3ccccc3)c3cc(Br)ccc3[nH]c2=O)CC1c1ccccc1Cl ; O=C(O)CCC(=O)N1N=C(c2c(-c3ccccc3)c3cc(Br)ccc3[nH]c2=O)CC1c1ccccc1 ; C=C(Br)/C=c1/c(-c2ccccc2)c(C2=NN(C(=O)CCC(=O)O)C(c3ccccc3F)C2)c(=O)[nH]c1=C ; O=C(O)CCC(=O)N1N=C(c2c(-c3ccccc3)c3cc(Br)ccc3[nH]c2=O)CC1c1ccc(F)cc1]

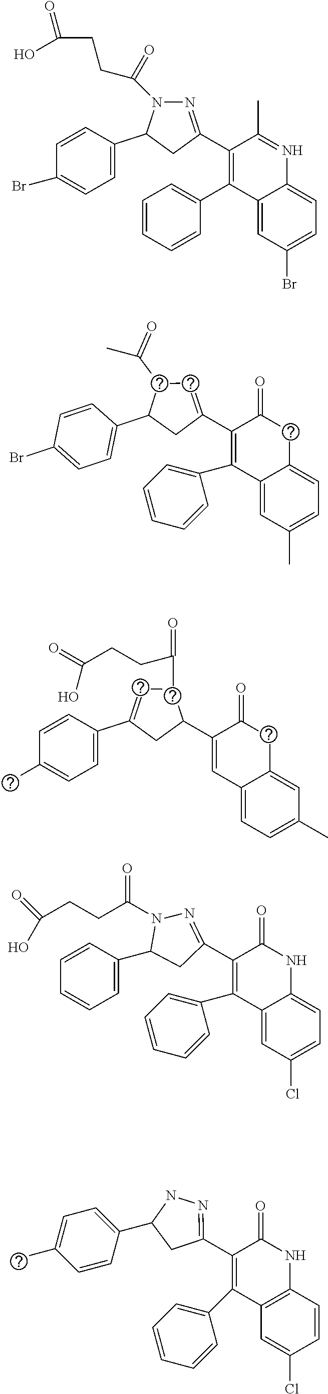

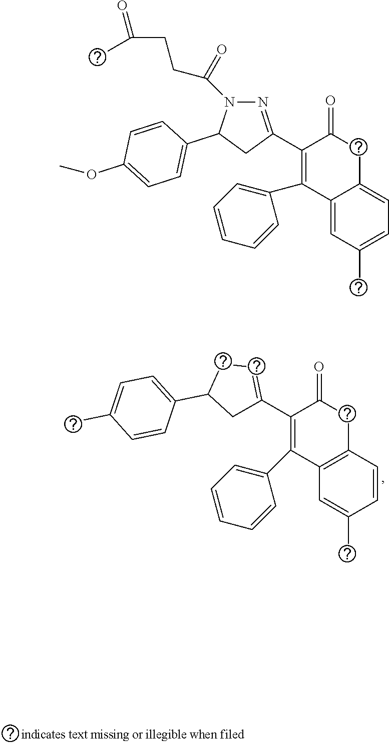 [SMILES: COc1ccc(C2CC(C3=C(c4ccccc4)c4cc(C)ccc4CC3=O)=NN2C(=O)CCC(C)=O)cc1 ; Cc1ccc(C2CC=C(C3=C(c4ccccc4)c4cc(C)ccc4CC3=O)C2)cc1]

including pharmaceutically acceptable salts, prodrugs, and esters thereof.

In further embodiments, the NMDA receptor antagonist is a compound according to:

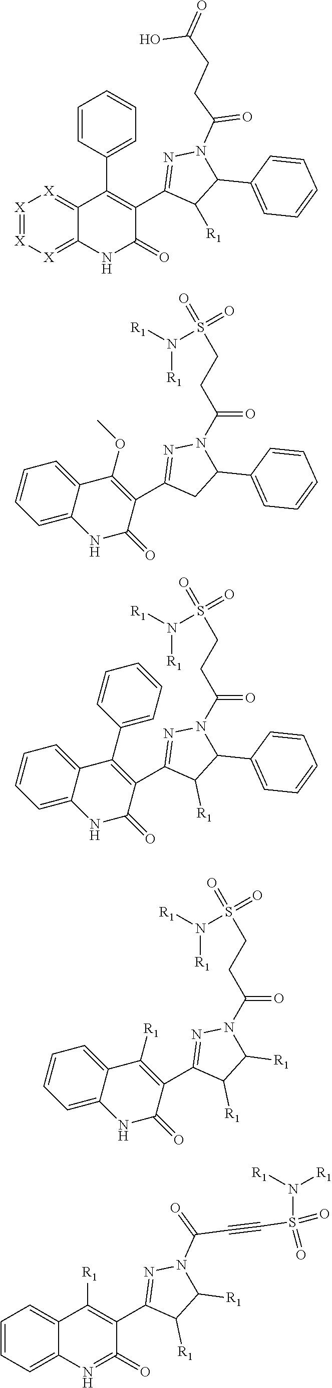

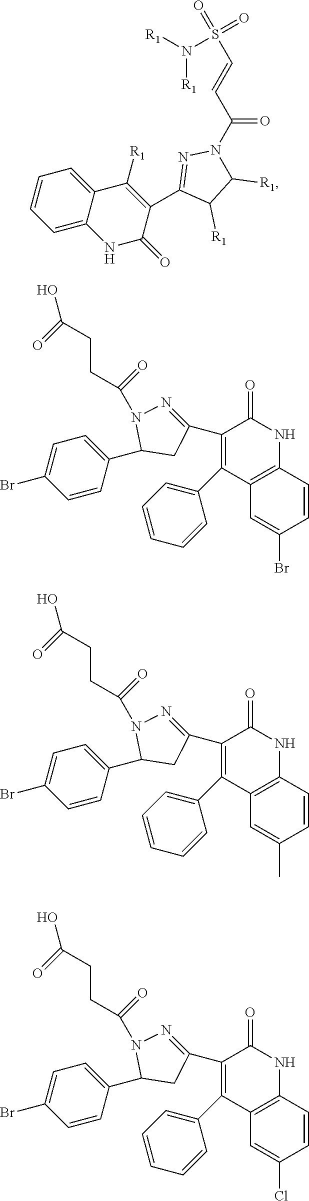

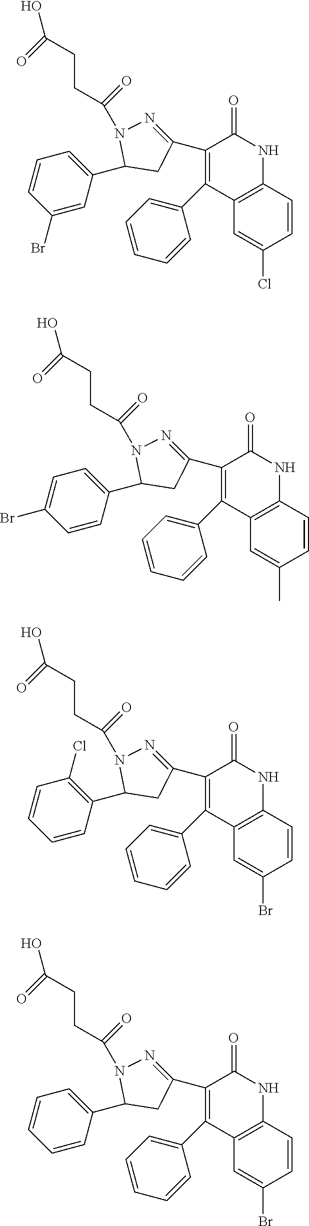 [SMILES: O=C(O)CCC(=O)N1N=C(c2c(-c3ccccc3)c3cc(Cl)ccc3[nH]c2=O)CC1c1cccc(Br)c1 ; Cc1ccc2[nH]c(=O)c(C3=NN(C(=O)CCC(=O)O)C(c4ccc(Br)cc4)C3)c(-c3ccccc3)c2c1 ; O=C(O)CCC(=O)N1N=C(c2c(-c3ccccc3)c3cc(Br)ccc3[nH]c2=O)CC1c1ccccc1Cl ; O=C(O)CCC(=O)N1N=C(c2c(-c3ccccc3)c3cc(Br)ccc3[nH]c2=O)CC1c1ccccc1]

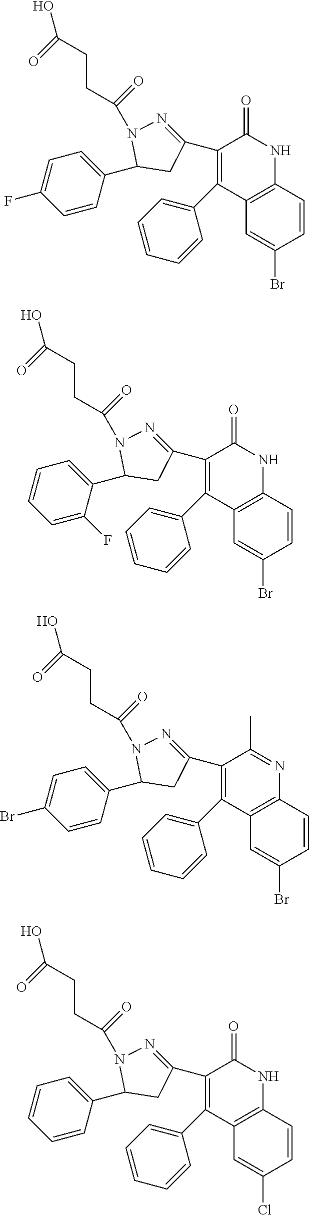 [SMILES: O=C(O)CCC(=O)N1N=C(c2c(-c3ccccc3)c3cc(Br)ccc3[nH]c2=O)CC1c1ccc(F)cc1 ; O=C(O)CCC(=O)N1N=C(c2c(-c3ccccc3)c3cc(Br)ccc3[nH]c2=O)CC1c1ccccc1F ; Cc1nc2ccc(Br)cc2c(-c2ccccc2)c1C1=NN(C(=O)CCC(=O)O)C(c2ccc(Br)cc2)C1 ; O=C(O)CCC(=O)N1N=C(c2c(-c3ccccc3)c3cc(Cl)ccc3[nH]c2=O)CC1c1ccccc1]

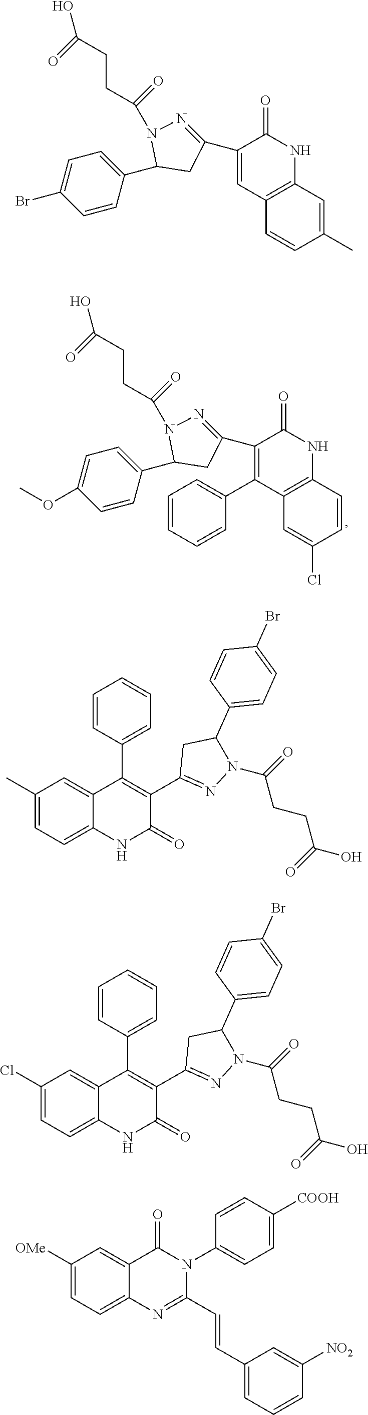

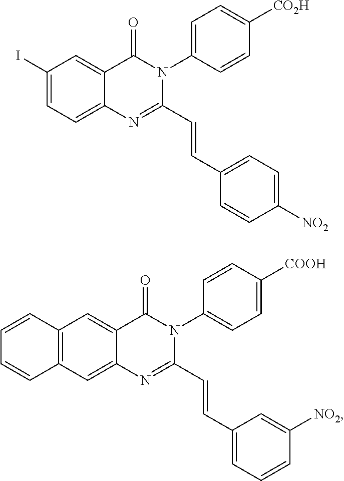

including pharmaceutically acceptable salts, esters, prodrugs and derivatives thereof.

In further embodiments, the NMDA receptor antagonist is a compound according to:

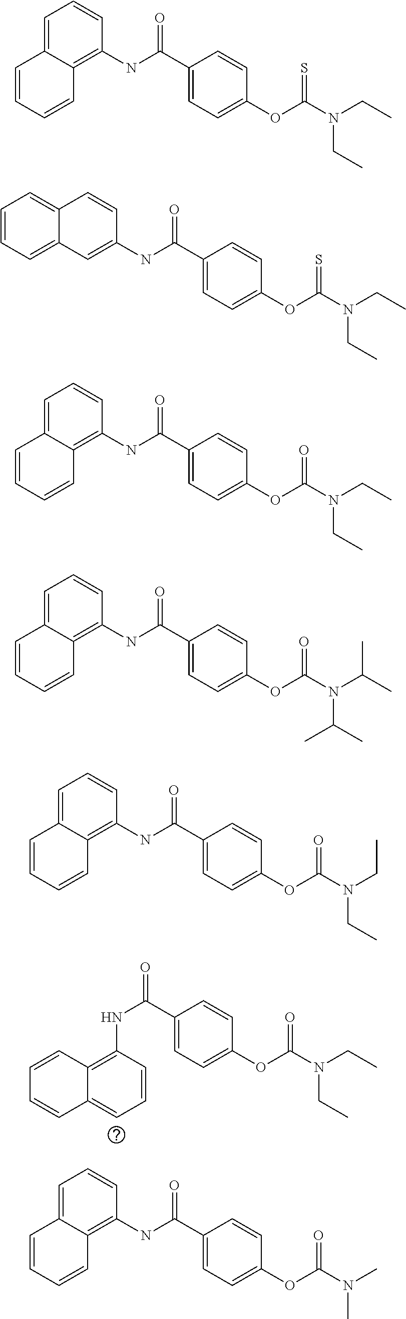

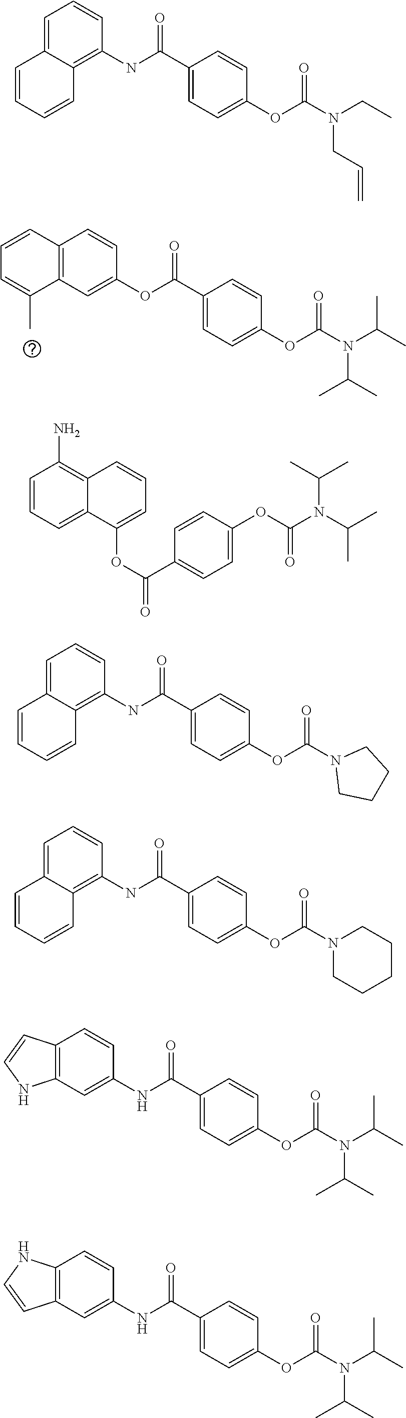

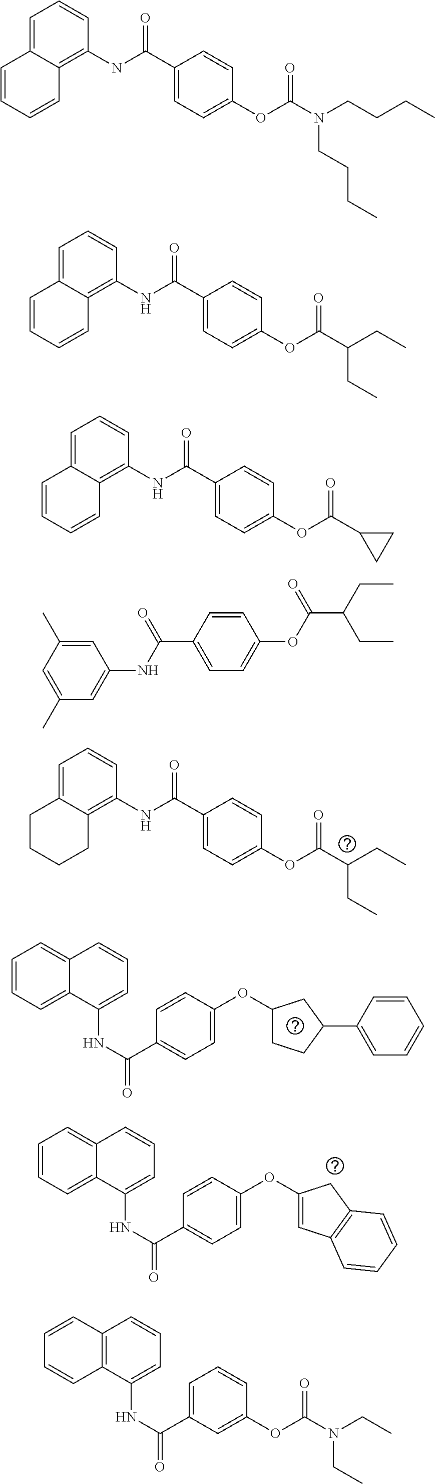

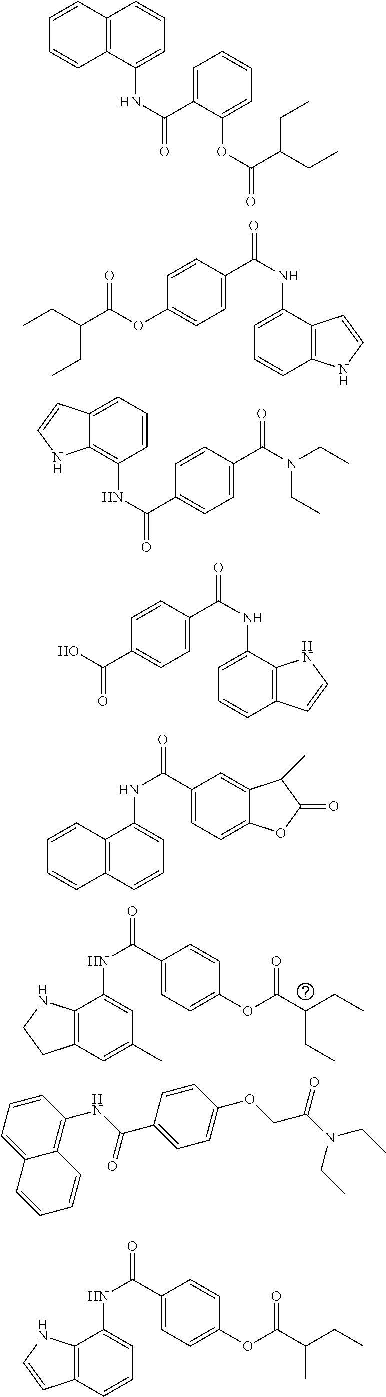

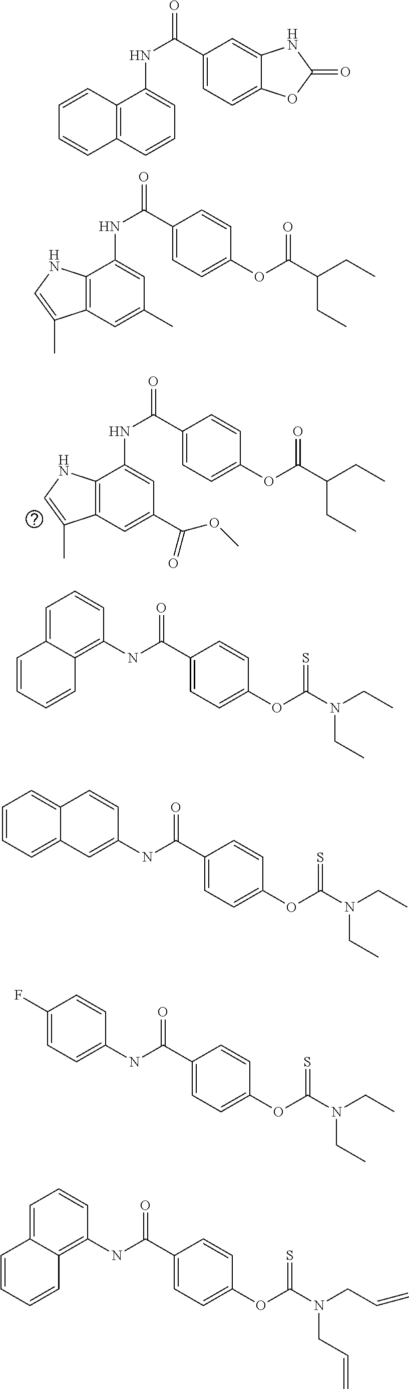

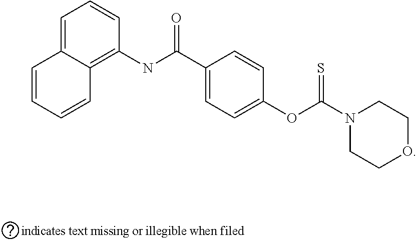 [SMILES: O=C(Nc1cccc2ccccc12)c1ccc(OC(=S)N2CCOCC2)cc1]

### Formula 210

In an embodiment, the NMDA receptor antagonist may be selected from one of the following compounds according to Formula 210. Such compounds are described in WO 2011/100585, published Aug. 18, 2011, corresponding to PCT/US2011/024583, filed Feb. 11, 2011, and U.S. Pat. No. 9,101,612, issued Aug. 11, 2015, which are each incorporated by reference in their entirety herein. In the case of any conflict of terminology of Formula 210, these references incorporated by reference herein control.

In an embodiment, the NMDA receptor antagonist is a compound according to Formula 210:

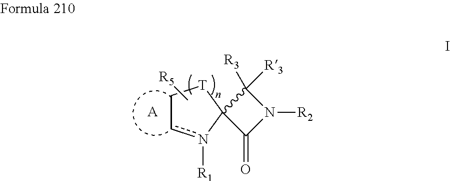

and pharmaceutically acceptable salts, stereoisomers, and N-oxides thereof; wherein Tis, independently for each occurrence, CR4R4, and n is 0, 1, 2 or 3;
A is optionally present and is selected from phenyl or pyridine, wherein A is optionally substituted by one or more substituents selected from Ra;
Ri is selected from the group consisting of H, hydroxyl, —S(O)2-C1-4alkyl; —SO2, C1-4alkyl, C2-C4alkenyl, phenyl, R7, or

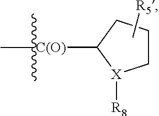

wherein C1-4 alkyl, C2-4alkenyl, or phenyl is optionally substituted by one or more substituents selected from Ra;
X is CH or N;
R3 and R3 are independently selected from the group consisting of H, halogen, hydroxyl, phenyl, C1-4alkyl, amido, amine, C2-4alkenyl, wherein C1-4alkyl, C2-4alkenyl, and phenyl are optionally substituted by one or more substituents selected from Ra;
R4 and R4 are independently selected from the group consisting of H, halogen, hydroxyl, phenyl, C1-4alkyl, amido, amine, C1-4alkoxy or C2-4alkenyl, wherein C1-4alkyl, C2-4alkenyl, C1-4alkoxy, and phenyl are optionally substituted by one or more substituents selected from Ra;
R2 is selected from the group consisting of H, R7, —S(O)2, S(O)2-C1-C4alkyl, C1-4alkyl, hydroxyl, or phenyl wherein C1-4alkyl, C2-4alkenyl and phenyl are optionally substituted by one or more substituents selected from Ra;
R5 and R5′ are each independently selected from group consisting of H, halogen, C1-4alkyl, C1-4alkoxy, C2-4alkenyl, cyano, amino, phenyl, and hydroxyl, wherein C1-4alkyl, C2-4alkenyl and phenyl are optionally substituted by one or more substituents selected from Ra;
R7 is selected from group consisting of —C(O)—R9, —C(O)—O—R9, or —C(O)—NRa-R9, R9 is selected from the group consisting of H, C1-4alkyl, phenyl, or heterocyclic, wherein C1-4alkyl, phenyl or heterocyclic is optionally substituted by 1, 2 or 3 substituents selected from Rb;
R8 is selected from group consisting of H, —C(O)—C1-4alkyl or C(O)—O—C1-4 alkyl, wherein C1-4alkyl is optionally substituted by 1, 2 or 3 substituents selected from Ra;
Ra is selected, independently for each occurrence, from carboxy, hydroxyl, halogen, amino, phenyl, C1-4alkyl, and C1-4alkoxy;
Rb is selected, independently for each occurrence, from the group consisting of carboxy, hydroxyl, halogen, amino, phenyl, C-14alkyl, C1-4alkoxy, and —NH—RC;
Rc is selected, independently for each occurrence from the group consisting of: —C(O)—O—C1-4alkyl; and —C(O)—C1-4alkyl; and
Rd is selected, independently for each occurrence, H and C1-4alkyl.

In further embodiments, the NMDA receptor antagonist is a compound according to:

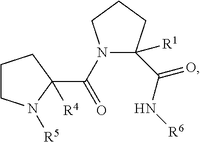

wherein R4 is H, R1 is benzyl, R5 is

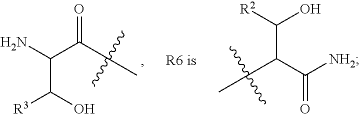

R2 is H or CH3, R3 is H or CH3, and stereoisomers, N-oxides or pharmaceutically acceptable salts thereof.

In further embodiments, the NMDA receptor antagonist is a compound according to:

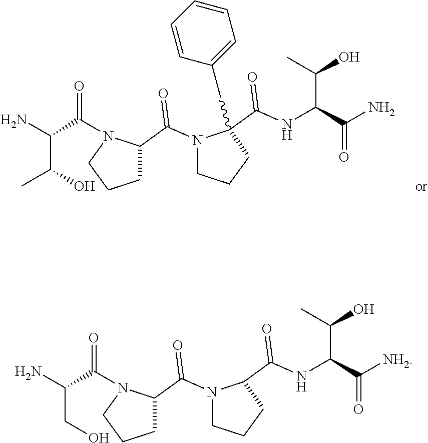

In further embodiments, the NMDA receptor antagonist is a compound according to:

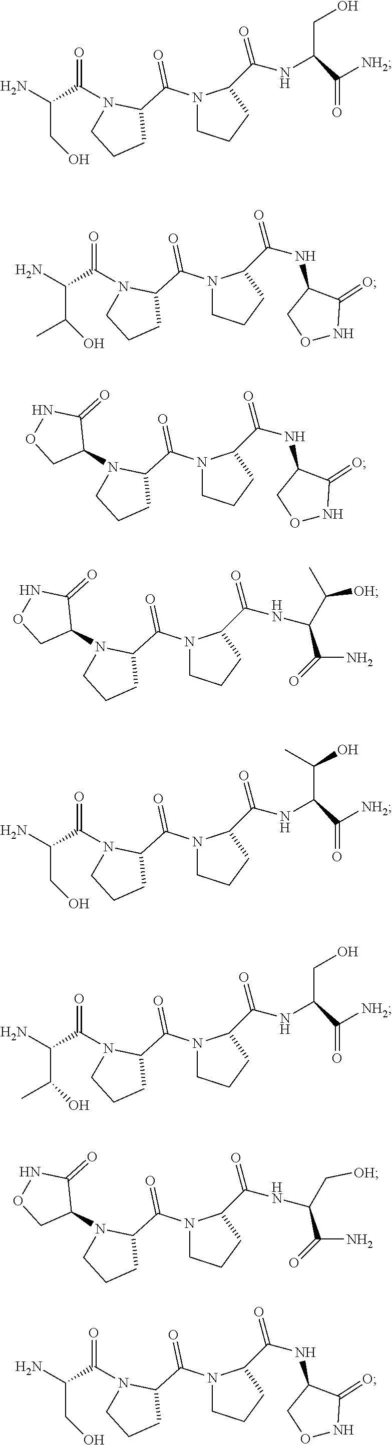

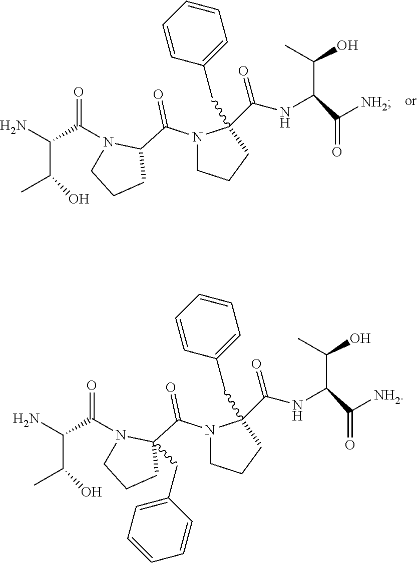

In an embodiment, the NMDA receptor antagonist is a compound according to:

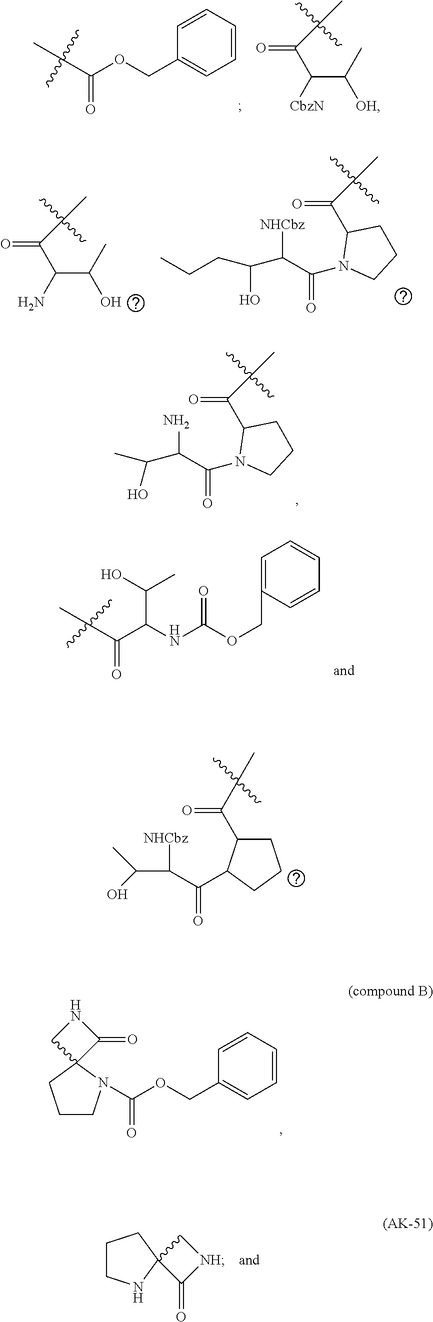

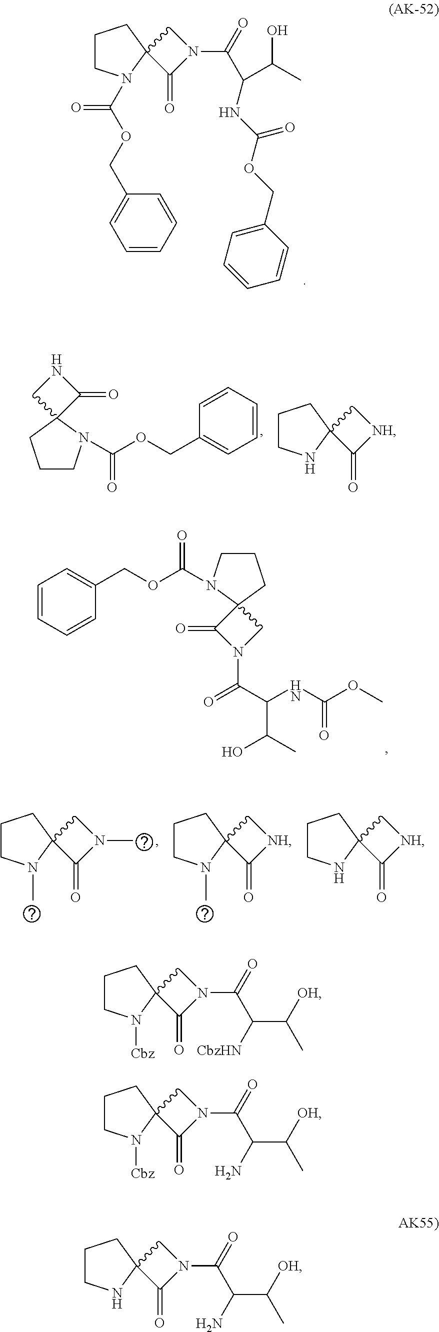

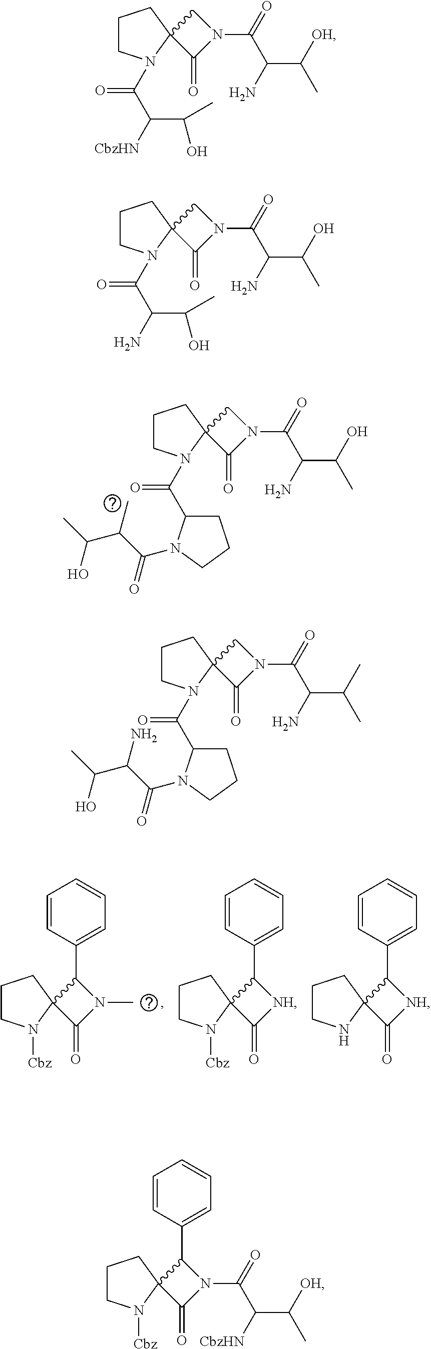

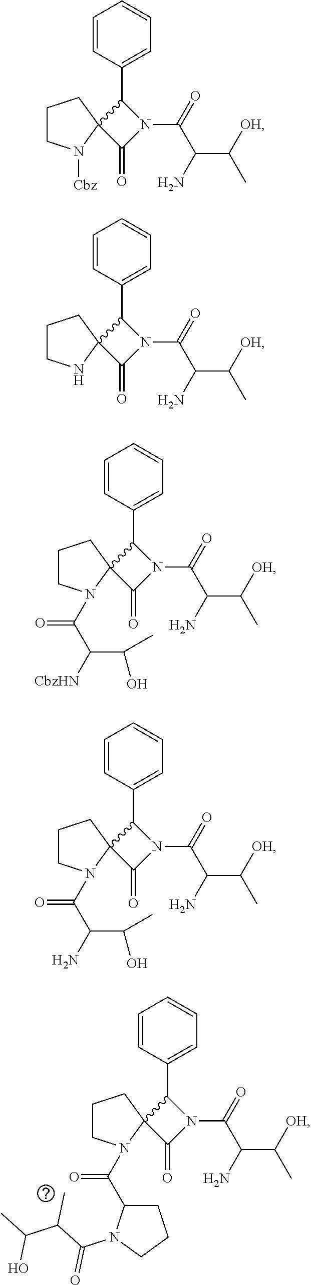

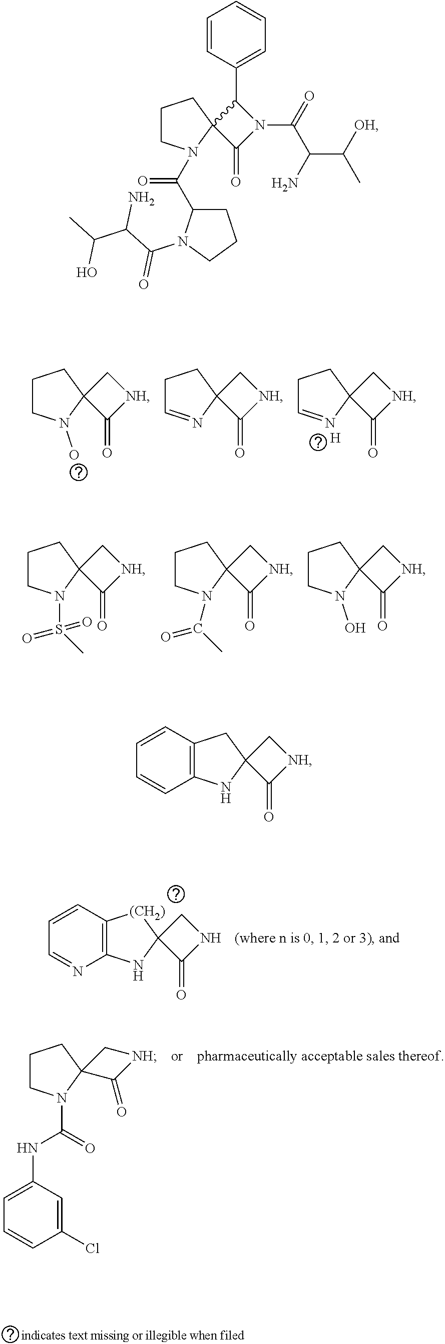

### Formula 211

In an embodiment, the NMDA receptor antagonist may be selected from one of the following compounds according to Formula 211. Such compounds are described in WO 2012/019106, published Feb. 9, 2012, corresponding to PCT/US2011/046756, filed Aug. 5, 2011, which is incorporated by reference in its entirety herein. In the case of any conflict of terminology of Formula 211, this references incorporated by reference herein controls.

In an embodiment, the NMDA receptor antagonist is a compound according to formulas 211 a-c:

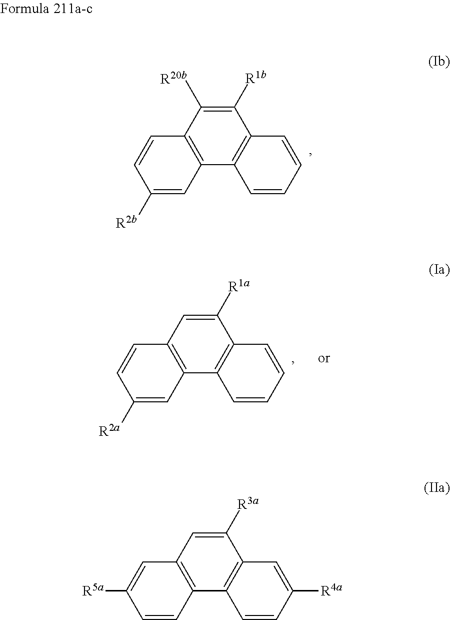

wherein R1b is iodo, alkyl, alkenyl, alkynyl, alkoxy, amino, cycloalkyl, aryl, heteroaryl, alkyleneamino, alkylene-CO2H, alkenylene-CO2alkyl, alkylene-OH, alkylene-cycloalkyl, alkylenearyl, alkenylenearyl, CHO, or thioalkyl;
R2b and R5a are each independently R^{A}, alkylene-R^{A}, NH2, C(O) NH-alkylenearyl, C(O) NH-alkyleneheteroaryl, C(O) NH-alkylene-R^{A}, alkylene-OH, C(O)alkyl, or NO2;
R1a is halo;
R2a is C(O)alkylene-NH2 or C(O)alkylene-heteroaryl;
R3a is H or halo;
R4a is H, heteroaryl, alkenylene-CO2H, or alkenylene-CO2-alkyl;
R20b is alkyl, alkenyl, alkynyl, NO2, amino, aryl or heteroaryl; and
RA is CO2H, CO2alkyl, P(O)(OH)(OH), P(O)(OH)(Oalkyl), P(O)(Oalkyl)(Oalkyl), P(O)(alkyl)(OH), SO2H, SO3H, C(O)NHOH, SO2NH2, or tetrazole, optionally with the proviso that R3a and R4a cannot each be H, or a salt, ester, solvate, tautomer, enantiomer, or hydrate thereof.

In further embodiments, the NMDA receptor antagonist is a compound selected from:

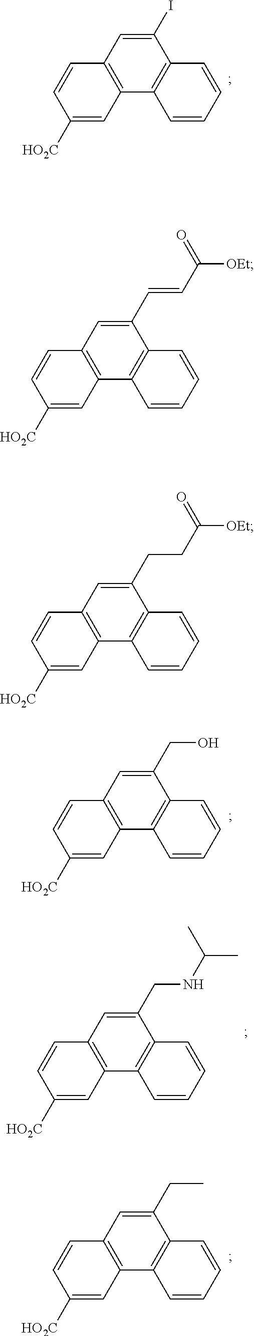 [SMILES: O=C(O)c1ccc2cc(I)c3ccccc3c2c1 ; CCOC(=O)/C=C/c1cc2ccc(C(=O)O)cc2c2ccccc12 ; CCOC(=O)CCc1cc2ccc(C(=O)O)cc2c2ccccc12 ; O=C(O)c1ccc2cc(CO)c3ccccc3c2c1 ; CC(C)NCc1cc2ccc(C(=O)O)cc2c2ccccc12 ; CCc1cc2ccc(C(=O)O)cc2c2ccccc12]

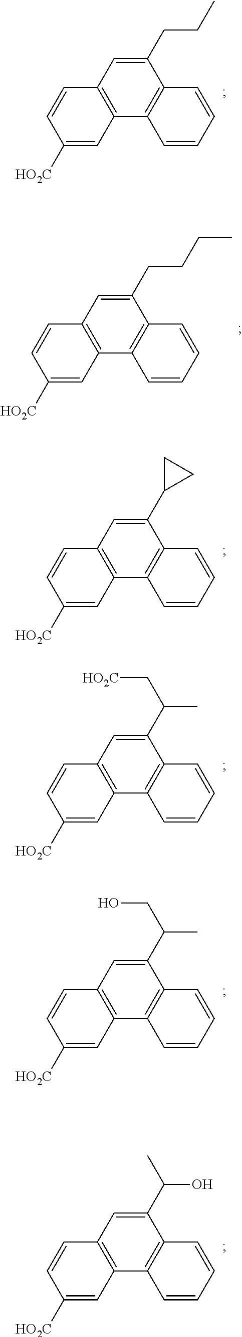 [SMILES: CCCc1cc2ccc(C(=O)O)cc2c2ccccc12 ; CCCCc1cc2ccc(C(=O)O)cc2c2ccccc12 ; O=C(O)c1ccc2cc(C3CC3)c3ccccc3c2c1 ; CC(CC(=O)O)c1cc2ccc(C(=O)O)cc2c2ccccc12 ; CC(CO)c1cc2ccc(C(=O)O)cc2c2ccccc12 ; CC(O)c1cc2ccc(C(=O)O)cc2c2ccccc12]

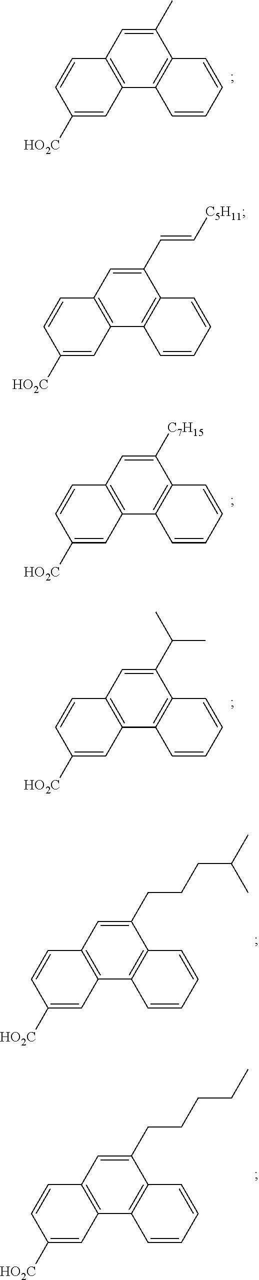

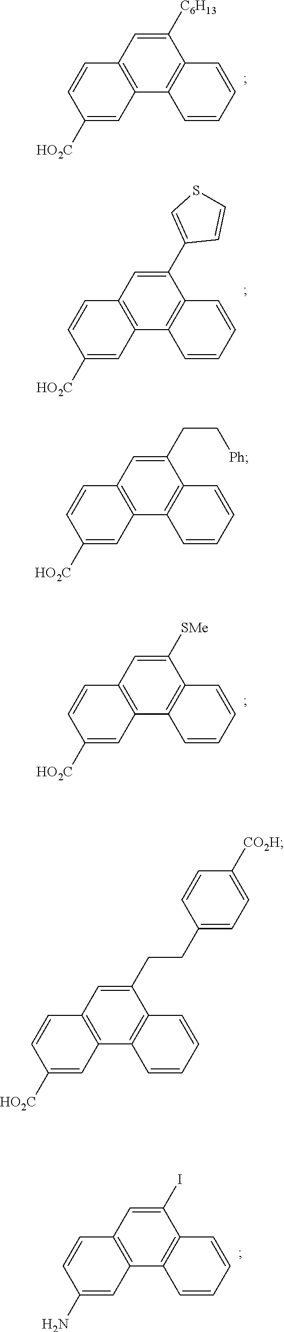

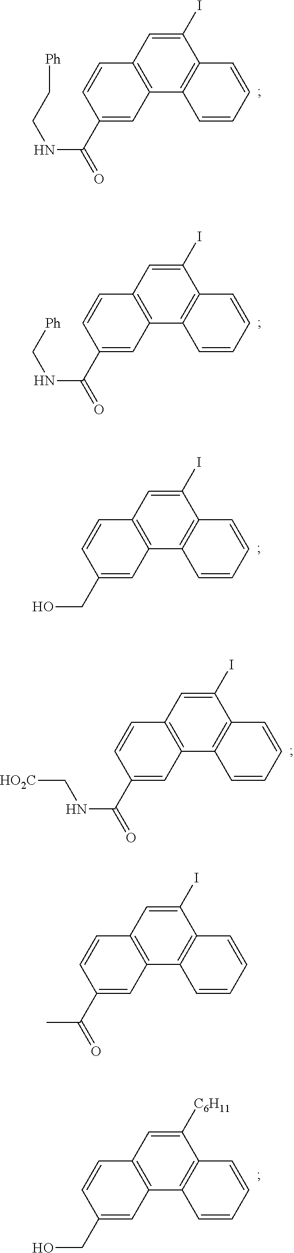

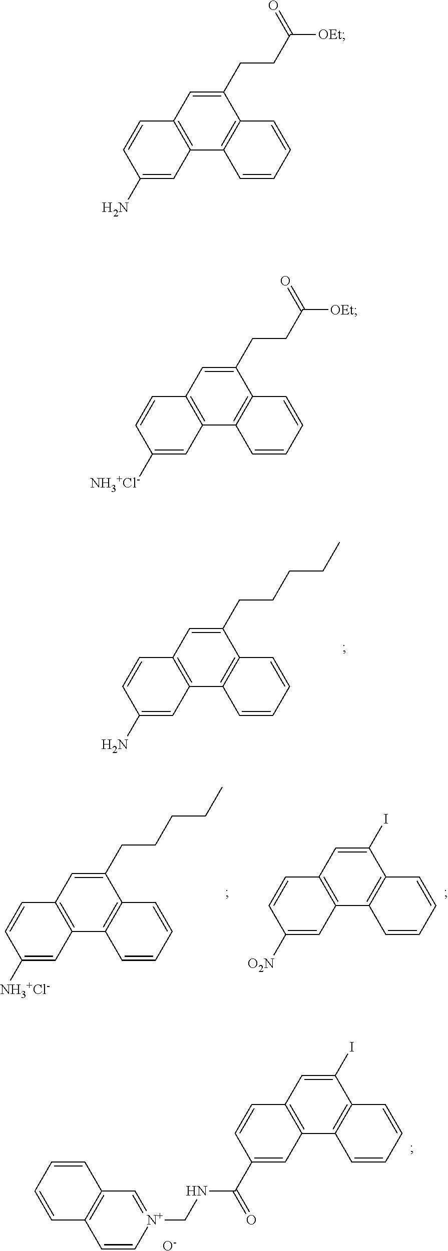

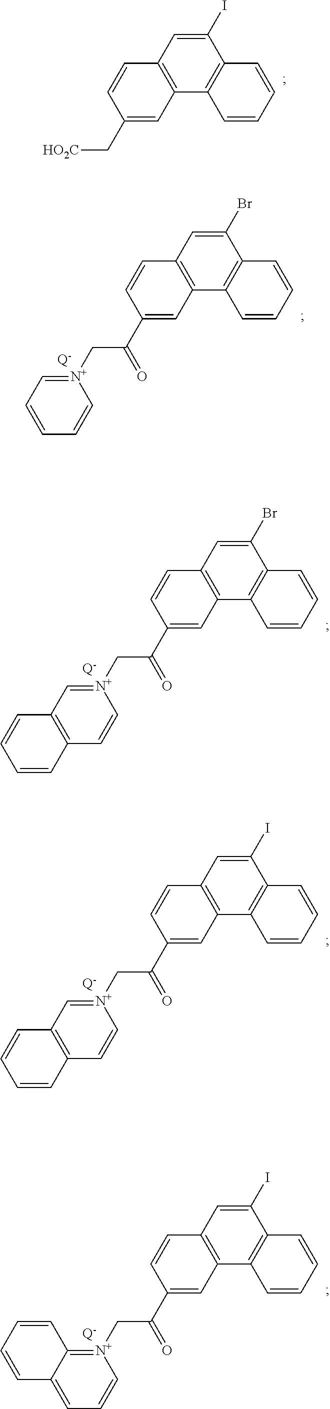

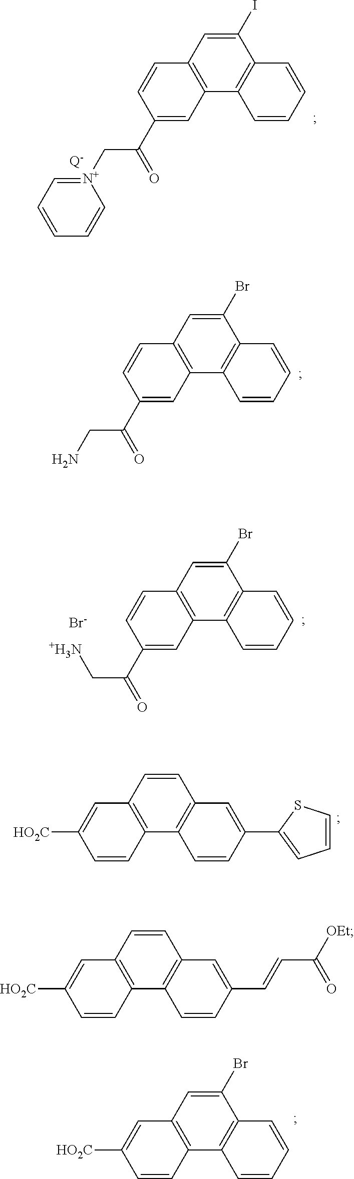

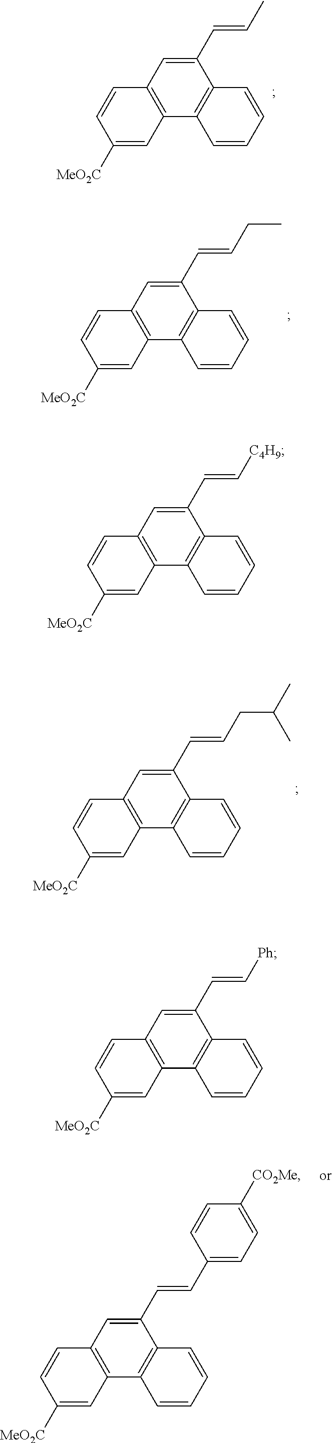

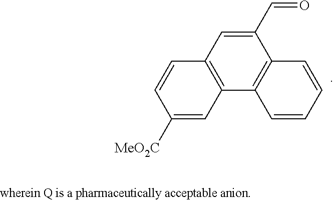 [SMILES: COC(=O)c1ccc2cc(C=O)c3ccccc3c2c1]

In yet further embodiments, the NMDA receptor antagonist is a compound according to Formula 212d-e:

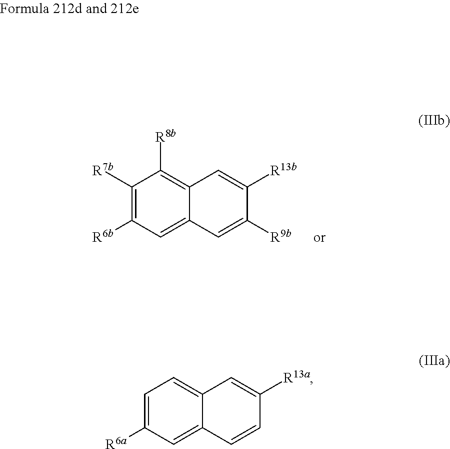

wherein
R6b is RA or alkylene-RA;
R7b is OH, alkoxy, or CO2H;
R8b is H, halo, or C4-C20alkyl;
R9b is halo, alkenyl, aryl, or alkyl;
R13b is H, C4-C2oalkyl, alkenyl, aryl, heteroaryl, alkeylenearyl, or alkenylenearyl;
R13a is C4-C20alkyl, alkenyl, alkenylene-CO2H, alkenylene-CO2alkyl, O-alkylene-CO2alkyl, or alkylenearyl;
R6a is RA, alkylene-RA, or alkylene-OH;
RA is CO2H, P(O)(OH) (OH), P(O)(OH) (Oalkyl), P(O)(Oalkyl) (Oalkyl),
P(0O(alkyl)(OH), SO2H, SO3H, C(O)NHOH, SO2NH2, or tetrazole;
   or a salt, ester, solvate, tautomer, enantiomer, or hydrate thereof.

In further embodiments, the NMDA receptor antagonist is a compound selected from:

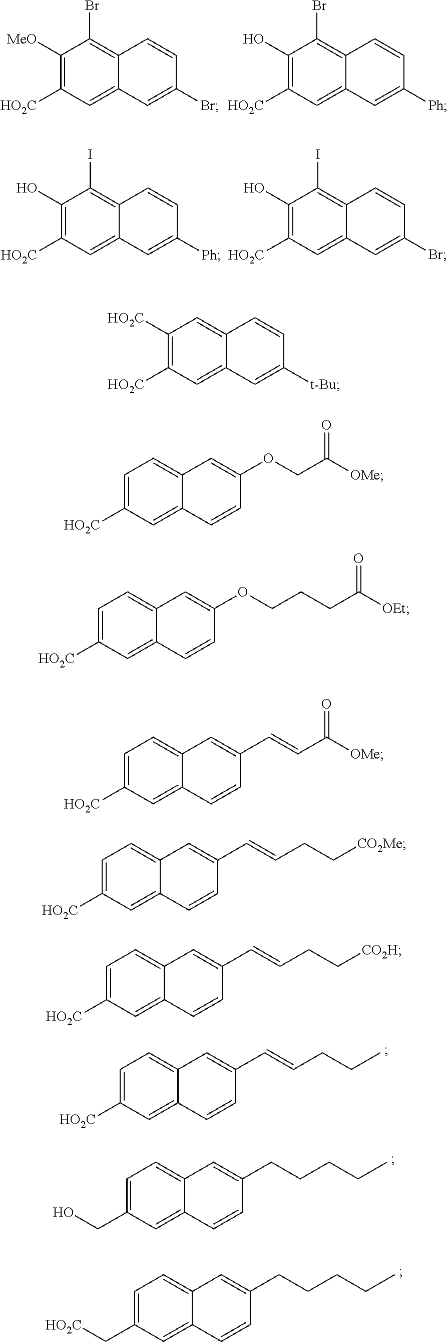

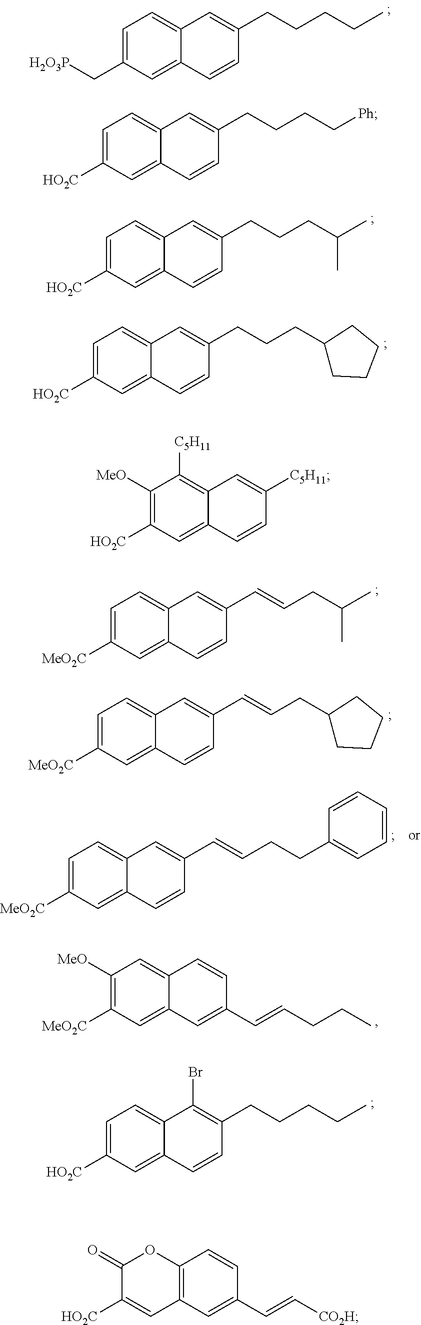

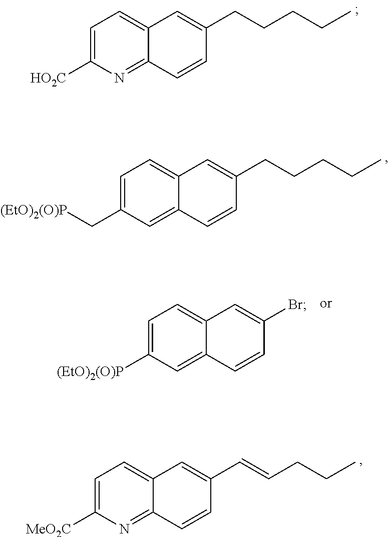

Or a salt, ester, solvate, tautomer, enantiomer, or hydrate thereof.

In further embodiments, the NMDA receptor antagonist is a compound according to:

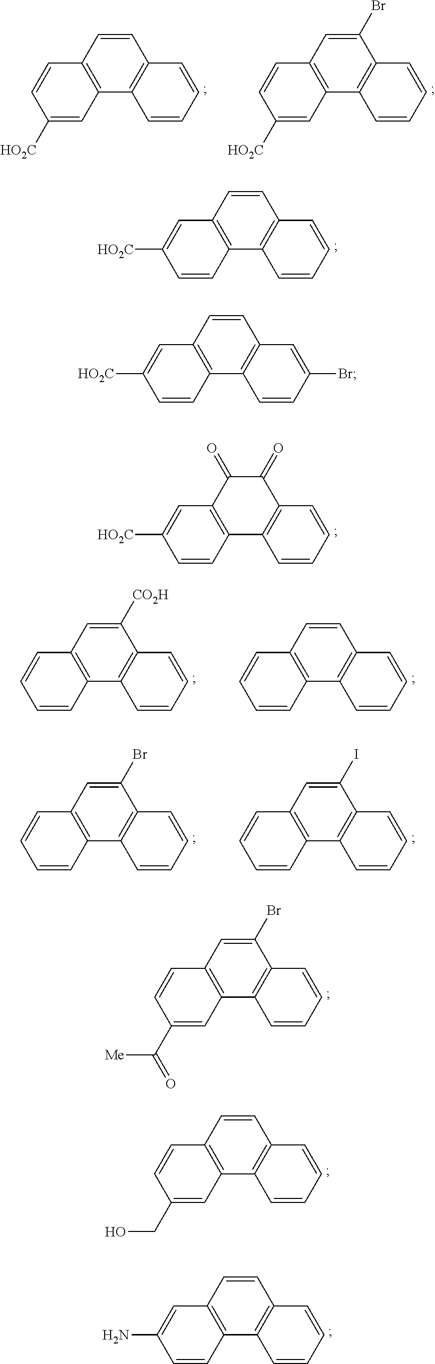

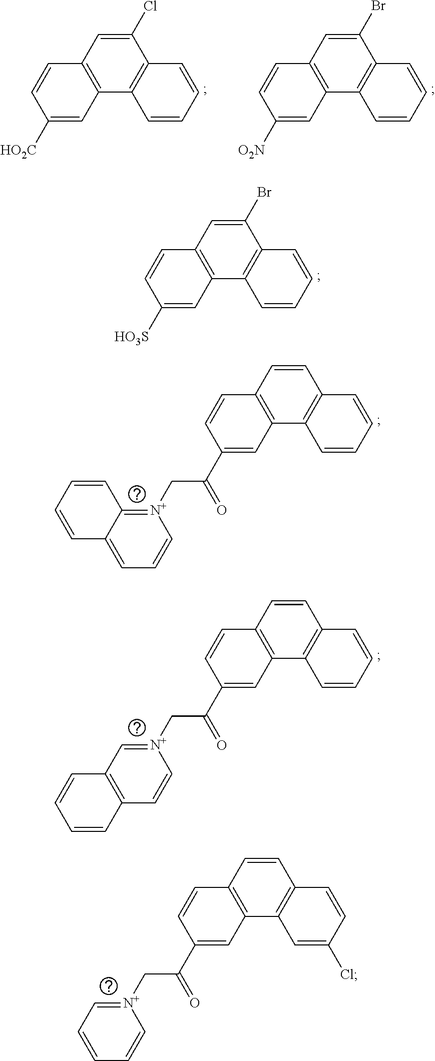

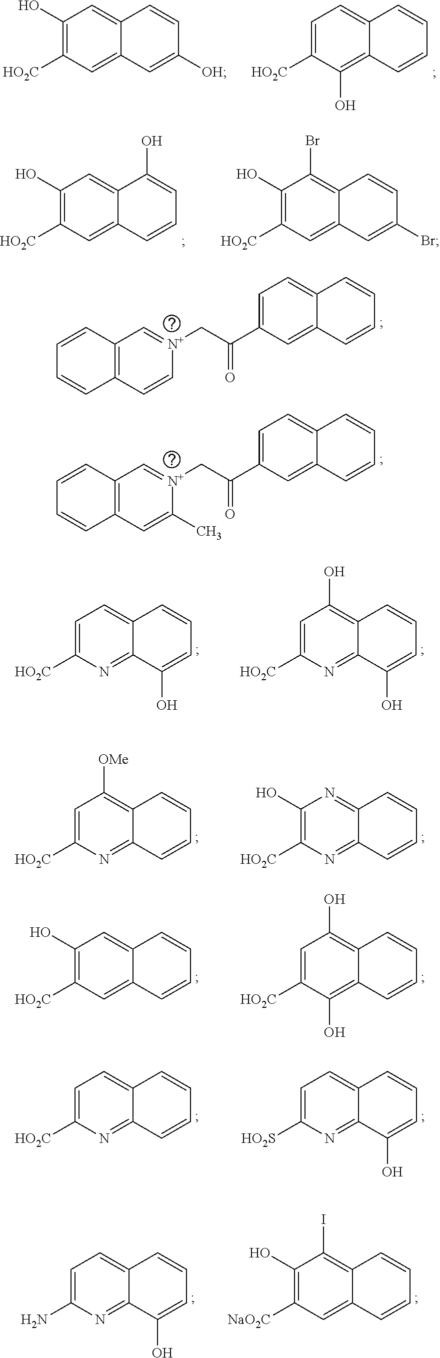

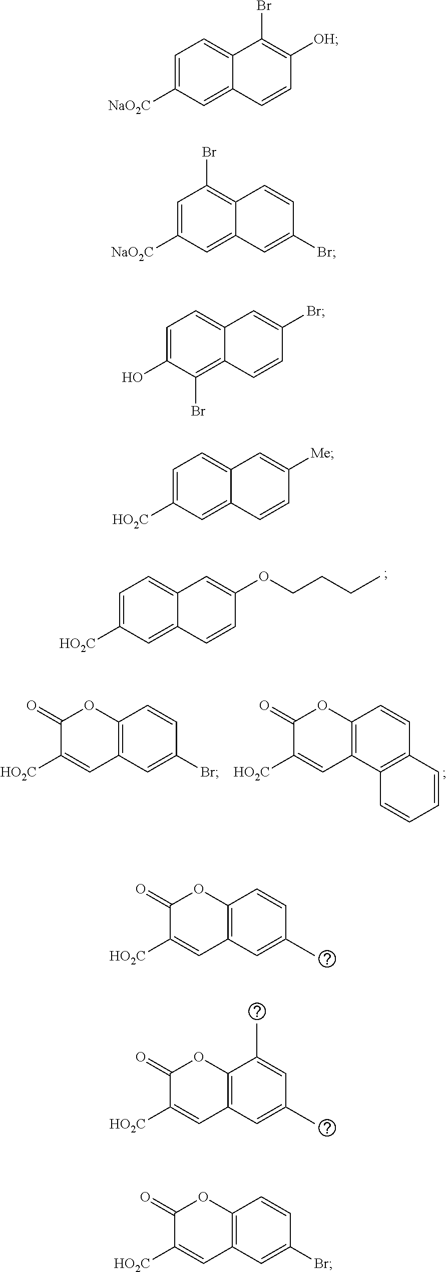

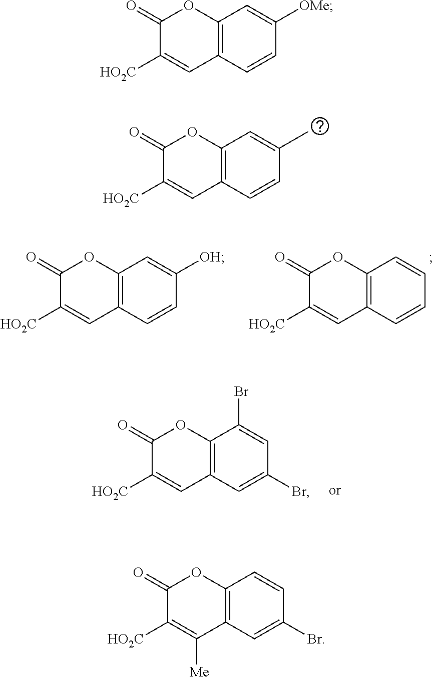

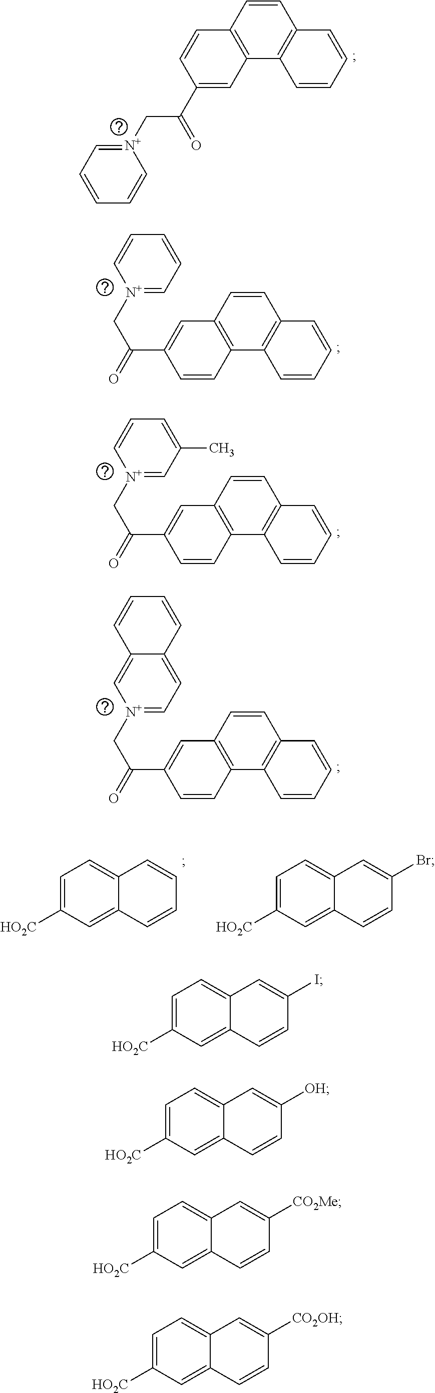

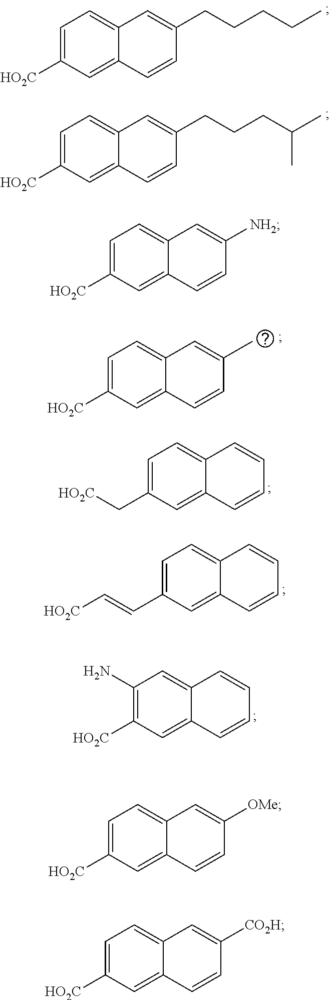

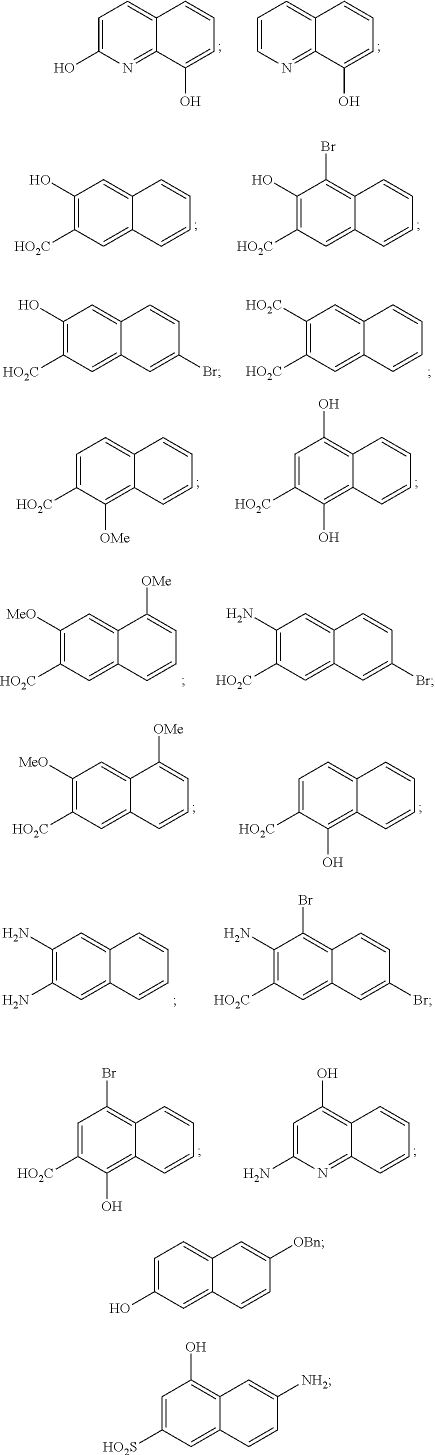

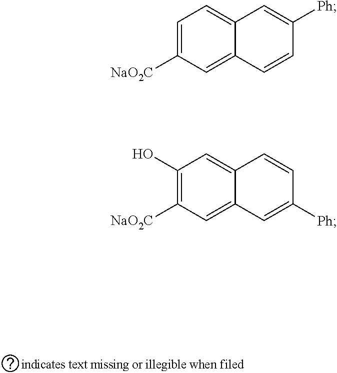

### Formula 212

In an embodiment, the NMDA receptor antagonist may be selected from one of the following compounds according to Formula 212. Such compounds are described in US 2011/0206780 published Aug. 25, 2011 corresponding to U.S. Ser. No. 12/984,989 filed Jan. 5, 2011 which is incorporated by reference in its entirety herein. In the case of any conflict of terminology of Formula 212, this reference incorporated by reference herein controls.

In an embodiment, the NMDA receptor antagonist is dextromethorphan:

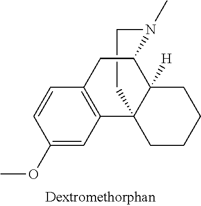 [SMILES: COc1ccc2c(c1)[C@]13CCCC[C@@H]1[C@H](C2)N(C)CC3]

In further embodiments, the NMDA receptor antagonist is dextromethorphan or a dextromethorphan analogue having one or more H replaced with deuterium (D).

In further embodiments, the NMDA receptor antagonist is a compound according to Formula 212:

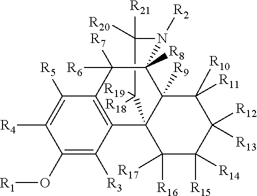

or a salt thereof, wherein:
R1 and R2 are independently selected from the group consisting of —CH3, —CH2D, —CD2H, and —CD3;
R3-R25 are independently selected from the group consisting of hydrogen and deuterium;
at least one of R3-R25 is deuterium; and
   if R6-R8 and R20-R21 are deuterium and R2 is —CD3, then at least one of R1-R5, R9-R19, and R22-R25 is deuterium or contains deuterium.

In further embodiments, the NMDA receptor antagonist is a compound selected from:

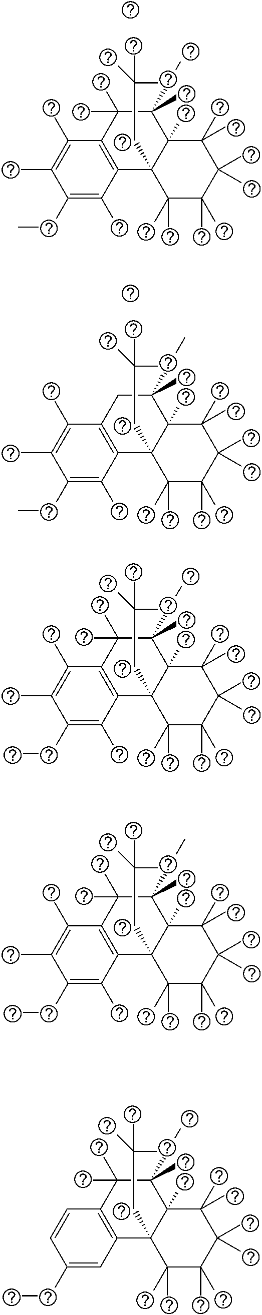

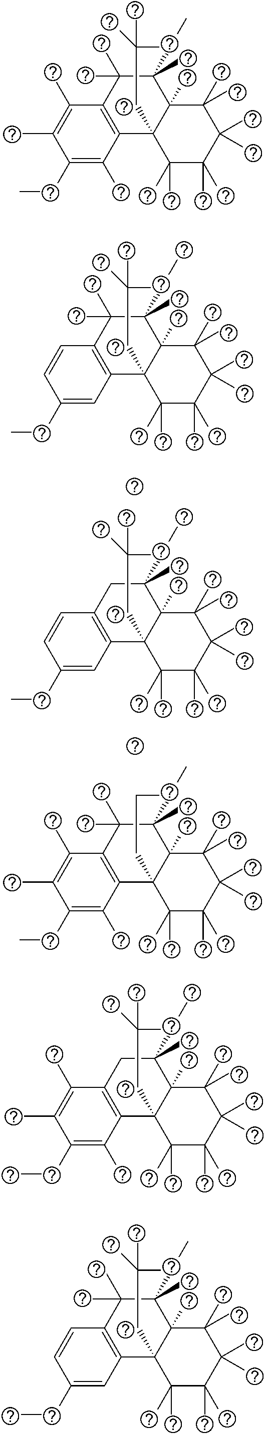

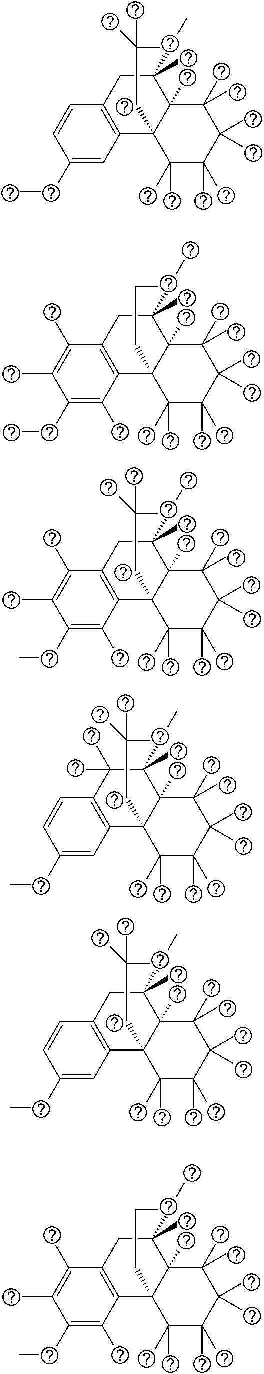

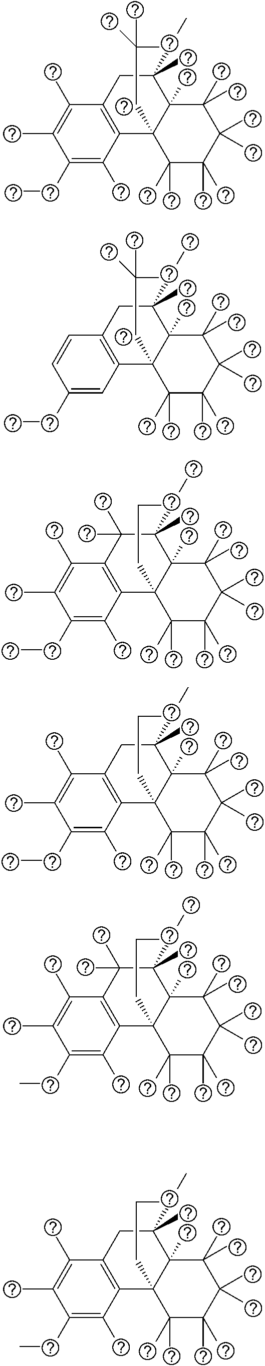

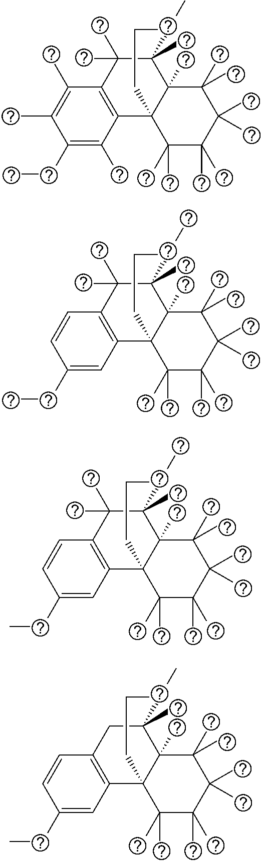

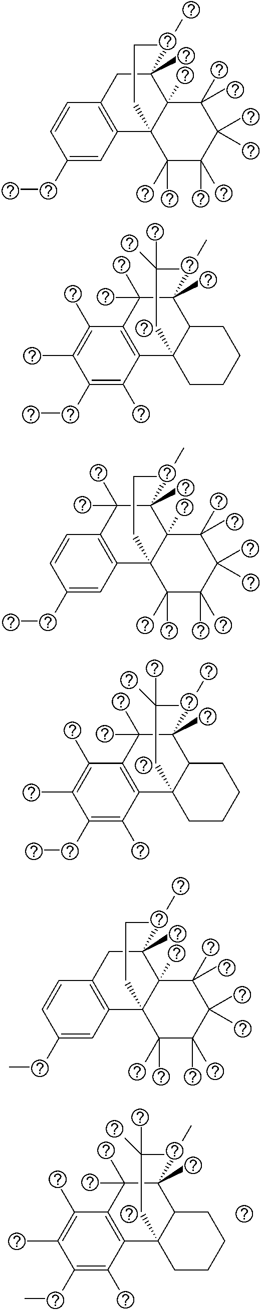

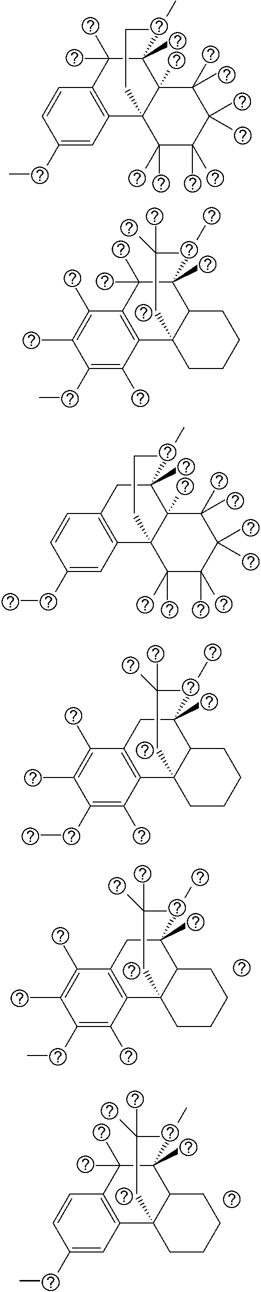

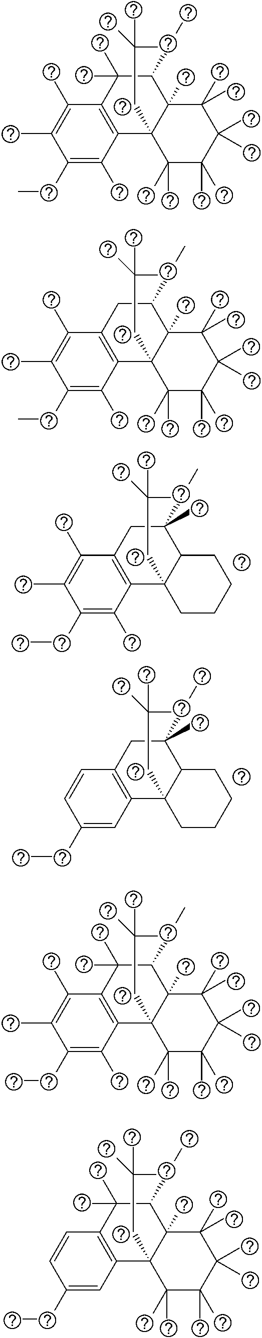

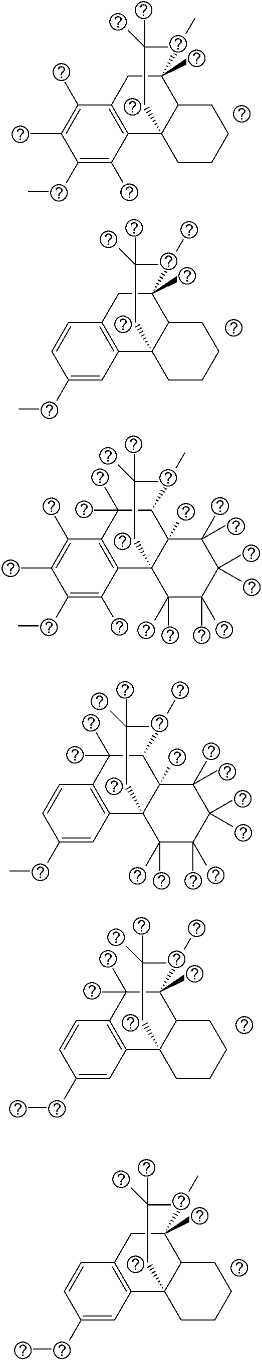

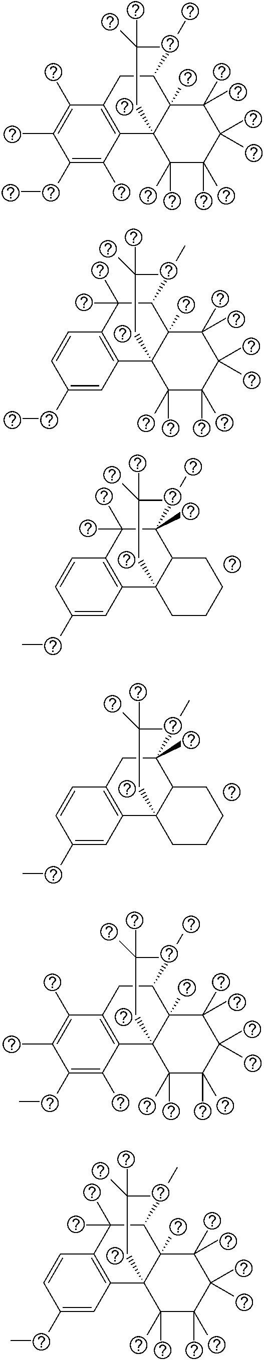

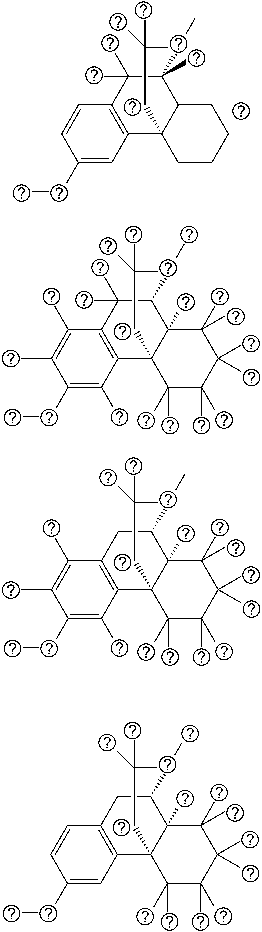

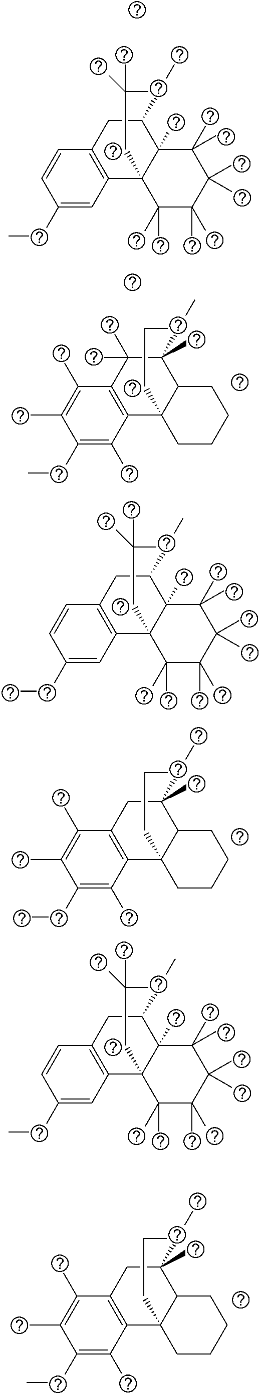

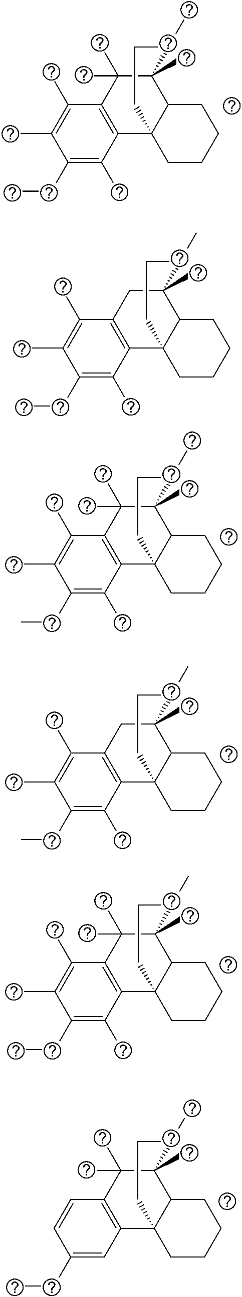

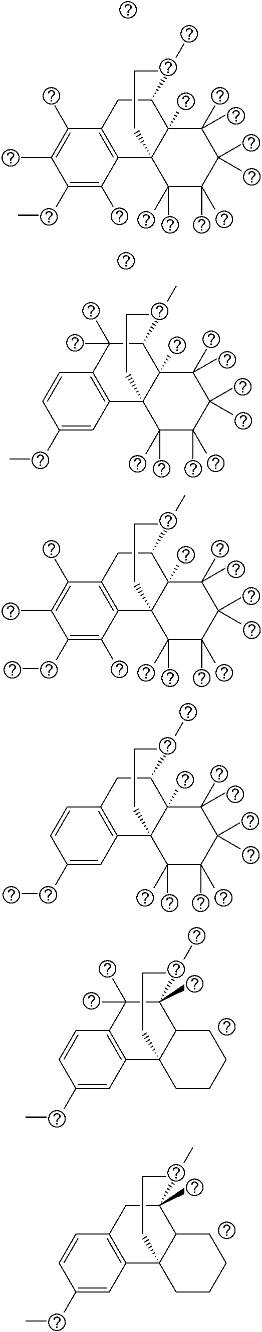

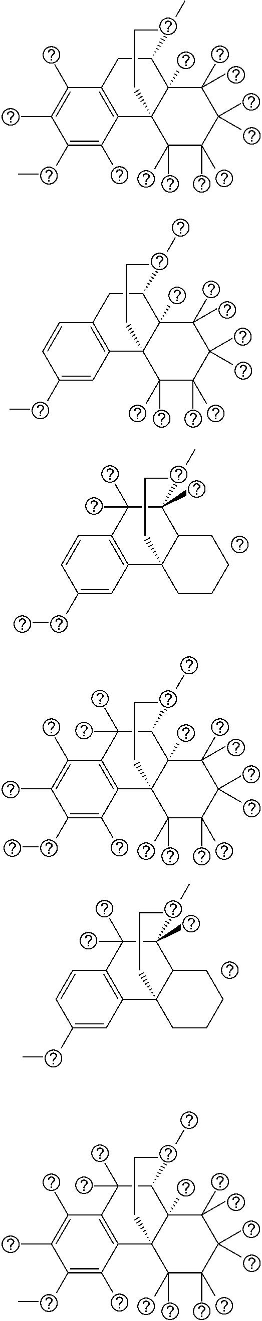

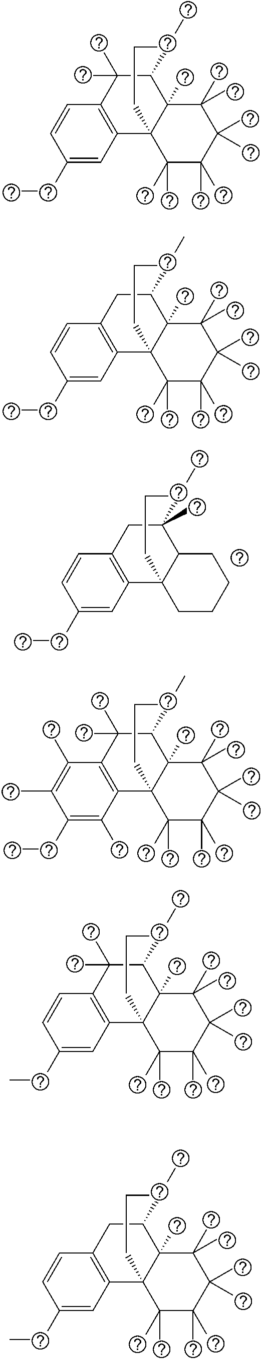

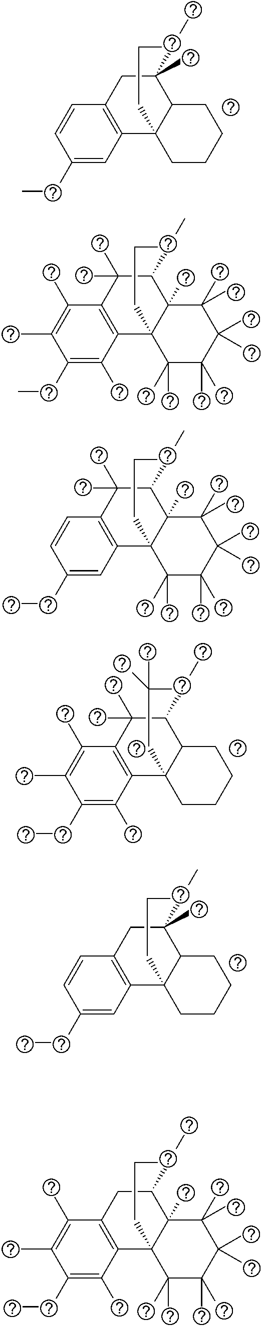

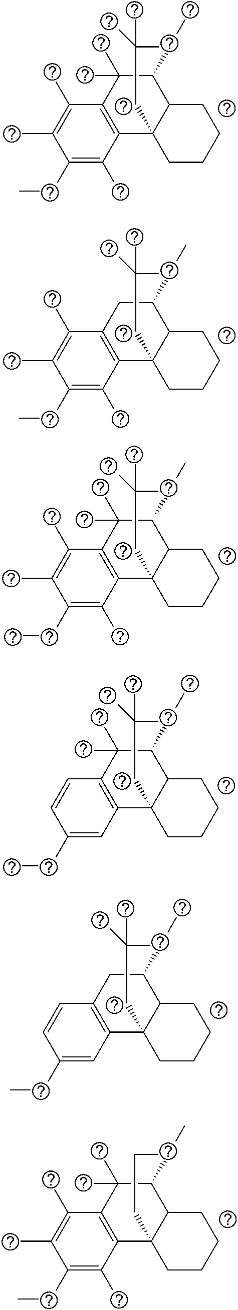

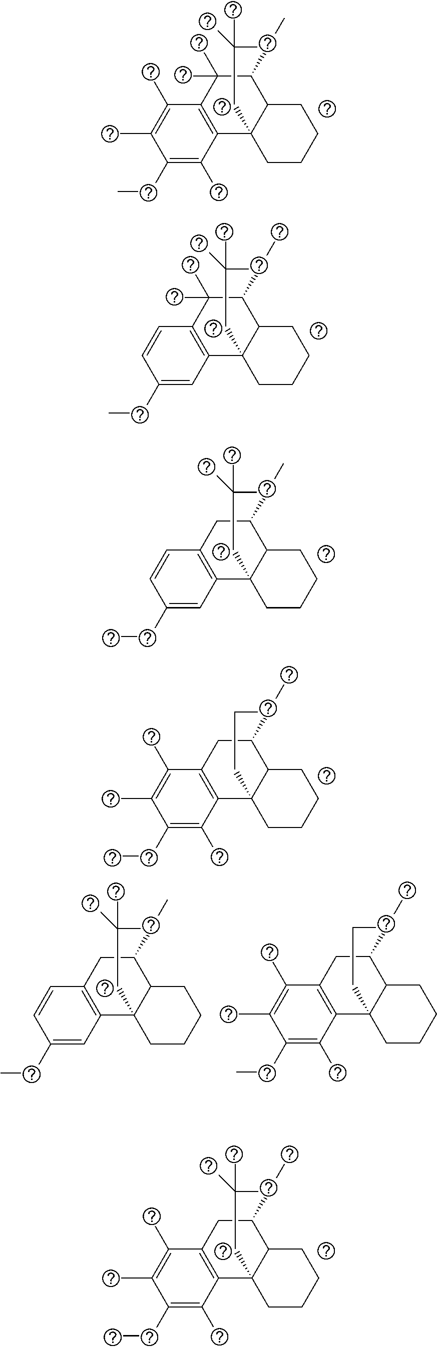

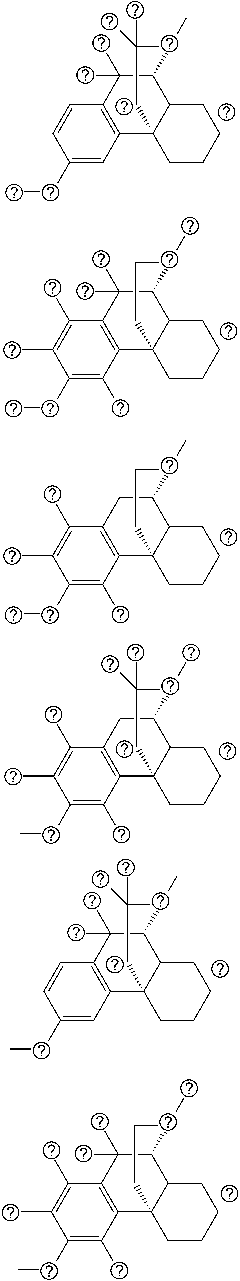

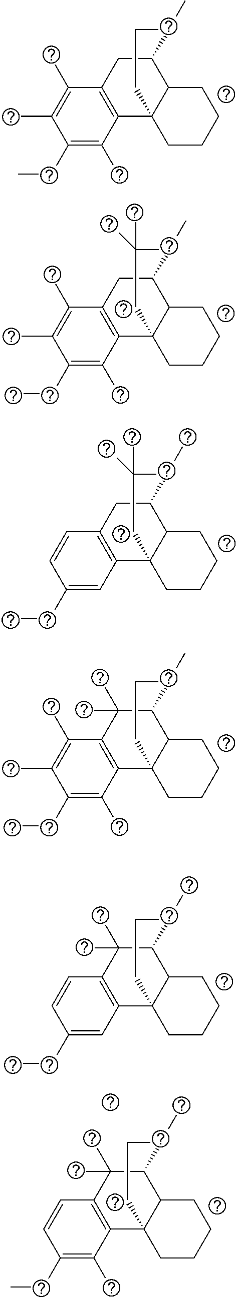

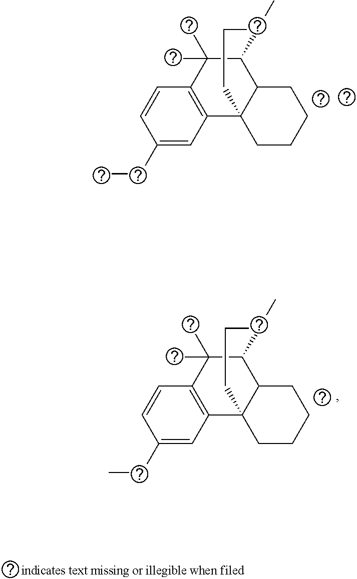

including mixtures thereof and at least about 10, 50, 90, or 98% deuterium enrichment.

### Formula 213

In an embodiment, the NMDA receptor antagonist may be selected from one of the following compounds according to Formula 213. Such compounds are described in, which are each incorporated by reference in their entirety herein. In the case of any conflict of terminology of Formula 213, these references incorporated by reference herein control.

In an embodiment, the NMDA receptor antagonist is a derivative of quinazoline, i.e. the following molecule portion, substituted in any manner:

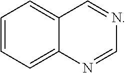

In an embodiment the NMDA receptor antagonist is:

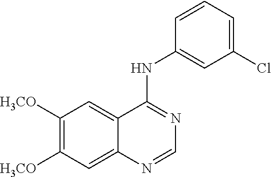 [SMILES: COc1cc2ncnc(Nc3cccc(Cl)c3)c2cc1OC]

or a pharmaceutically acceptable salt or derivative thereof.

In an embodiment, the NMDA receptor antagonist is a compound according to:

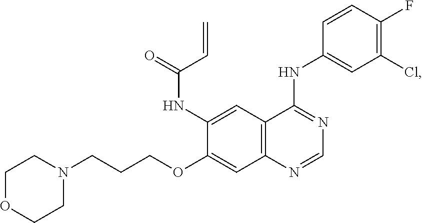 [SMILES: C=CC(=O)Nc1cc2c(Nc3ccc(F)c(Cl)c3)ncnc2cc1OCCCN1CCOCC1]

or a pharmaceutically acceptable salt or derivative thereof.

In further embodiments, the NMDA receptor antagonist is a compound according to:

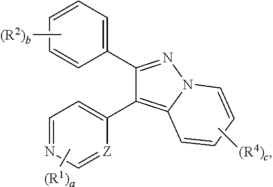

wherein Z is N; a is 1 or 2; b is 1, 2 or 3; c is 1, 2 or 3; each R1 is independently selected from groups of the formula ~(X)d~(CH2)e~R5 wherein d is 0 or 1; e is 0 to 6; Xis O, NR or S(O) f where fis 0, 1 or 2; R is hydrogen, halogen, Ci_6 alkyl, C2-6 alkyl, C2-6 alkenyl, C3-12 cycloalkyl, heterocyclyl, aryl, heteroaryl, hydroxyl, cyano, nitro, trihalomethyl, NR7R8, C5H4NR7R8, C5H4 (CH2) NR7R8, C(O)R7, C(O)NR7R8, OC(O)R7, OC(O)NR7R8, CO2R7, OCOR7, SO2R7, SO2NR7R8, C(═NR7) NR7R8, NR7 (C≡NR7) NR7R8, NHC(O)R7 or N(1-3alkyl) C(O)R7; each R2 is independently selected from hydrogen, cyano, halogen, trihalomethyl, OC2-5alkyl, C2-5alkyl, C2-5alkenyl, C2-5alkynyl, S(O)gC1-5 alkyl where g is 0, 1 or 2, NC1-5 alkyl(C1-5 alkyl), hydroxyl or nitro; each R4 is independently selected from groups of the formula

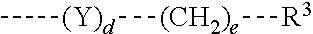

wherein d is 0 or 1; e is 0 to 6; Y is O or S(O) f where f is 0, 1 or 2; R3 is hydrogen, halogen, C1-6 alkyl, C2-5 alkyl, C2-5 alkenyl, C3-12 cycloalkyl, heterocyclyl, aryl, heteroaryl, hydroxyl, cyano, nitro, trihalomethyl, phthalamido, C5H4NR7R8, C5H4 (CH2) NR7R8, C(O)R7, C(O)NR7R8, OC(O)R7, OC(O)NR7R8, CO2R7, OCOR7, SO2R7, SO2NR7R8 or C(═NR7) NR7R8; R6 is H, C1-5 alkyl, C2-5 alkyl, C2-5 alkenyl, C3-12 cycloalkyl, or heterocyclyl; R7 and R8 are each independently H, C1-8 alkyl, C2-5 alkenyl, SO2C1-5 alkyl, (CH2)m-C3-12 cycloalkyl, (CH2)m-aryl, (CH2)m-heterocyclyl, (CH2)m-heteroaryl, wherein m=0, 1 or 2, or may, together with the nitrogen atom to which they are bound, form a heterocyclyl group; and wherein any of said alkyl, alkenyl and alkynyl groups may be optionally substituted with up to three members selected from halogen, hydroxyl, oxo, cyano, NR7R8, C1-6 alkyl, OC1-6 alkyl, S(O) C1-5 alkyl, S(O)2C1-5 alkyl and SO2NNR7R8; and wherein any of said cycloalkyl, heterocyclyl, aryl, and heteroaryl groups may be optionally substituted with substituents selected from a group consisting of C1-6alkyl, C1-6 alkoxy, C1-6 alkylsulfenyl, C1-6 alkylsulfinyl, C1-6 alkylsulfonyl, hydroxy, oxo, mercapto, nitro, cyano, halogen, C1-6 perfluoroalkyl, amino optionally substituted by C1-6 alkyl, carbamoyl optionally substituted by C1-6 alkyl, NR R, carboxy and aminosulfonyl optionally substituted by C1-6 alkyl.

In another embodiment, the NMDA receptor antagonist is a compound according to:

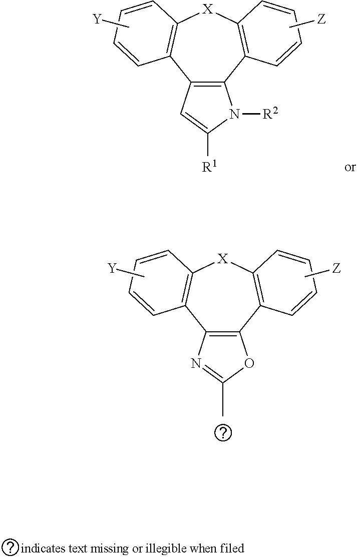

wherein X is O, S, S(═O), S(═O)2, or NRa, wherein Ra is hydrogen or a substituent selected from C1-C3-alkyl, C1-C3-alkanoyl, C1-C7-alkoxycarbonyl, C7-C10-arylalkyloxycarbonyl, C7-C10-aroyl, C7-C10-arylalkyl, C3-C7-alkylsilyl or C5-C10-alkylsilylalkyloxyalkyl; Y and Z independently from each other mean one or more identical or different substituents linked to any available carbon atom selected from the group consisting of hydrogen; halogen, C1-C4-alkyl, C2-C4-alkenyl, C2-C4-alkinyl, halo-C1-C4 alkyl, hydroxy, C1-C4-alkoxy, trifluoromethoxy, C1-C4-alkanoyl, amino, amino-C1-C4-alkyl, N—(C1-C4-alkyl)amino, N,N-di(C1-C4-alkyl)amino, thiol, C1-C4 alkylthio, sulfonyl, C1-C4 alkylsulfonyl, sulfinyl, C1-C4 alkylsulfinyl, carboxy, C1-C4 alkoxycarbonyl, cyano and nitro; R1 is hydrogen, CHO, (CH2)2COOH, (CH2)2CO2CH2CH3, (CH2) mL wherein m is an integer from 1 to 3, n is an integer between 0-3 and L is OH or Br, or a substituent set forth by formula VII:

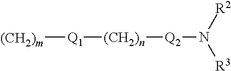

R2 and R3 are either or both hydrogen, C1-C4-alkyl, aryl such as an aromatic ring or fused aromatic rings containing one ring of at least 6 carbon atoms or two rings with a total of 10 carbon atoms and conjugated double bonds; or together with N are heterocyclic having 5, or 6-member rings or heteroaryl fully saturated or partly unsaturated heterocycle group containing at least one hetero atom such as O, S or N and where said heterocycle is optionally substituted with one or two substituents such as halogen, C1-C4-alkyl, cyano, nitro, hydroxy, C1-C4-alkoxy, thiol, C1-C4-alkylthio, amino, N—(C1-C4)alkylamino, N,N-di(C1-C4-alkyl)amino, sulfonyl, C1-C4 alkylsulfonyl, sulfinyl, C1-C4 alkylsulfinyl; and wherein heteroaryl relates to aromatic and partially aromatic groups of a monocyclic or bicyclic ring with 4 to 12 carbon atoms and at least one of O, S or N and where said heteroaryl is optionally substituted with one or two substituents which are selected from halogen, C1-C4-alkyl, cyano, nitro, hydroxy, C1-C4-alkoxy, thiol, C1-C4-alkylthio, amino, N—(C1-C4)alkylamino, N,N-di(C1-C4-alkyl)amino, sulfonyl, C1-C4 alkylsulfonyl, sulfinyl, C1-C4 alkylsulfinyl;

In one embodiment, Nis substituted with a heterocycle or heteroaryl that is morpholine-4-yl, piperidine-1-yl, pyrrolidine-1-yl, imidazole-1-yl or piperazine-1-yl

Qi and Q2 are either or both oxygen, CH2, sulfur or a group set forth by:

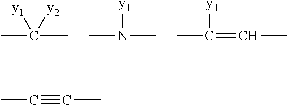

wherein yi and y2 are either or both; hydrogen, halogen (F, C1 or Br); C1-C4-alkyl, aryl wherein aryl has the meaning as defined above, —OH, C1-C4-alkyloxy, C1-C4 alkanoyl, thiol, C1-C4-alkylthio, sulfonyl, C1-C4 alkylsulfonyl, sulfinyl, C1-C4 alkylsulfinyl, cyano, nitro or together form carbonyl or imino group, and of their pharmaceutically acceptable salts and solvates for the manufacture of pharmaceutical formulations for the treatment and prevention of diseases, damages and disorders of the central nervous system caused by disorders of neurochemical equilibrium of biogenic amines or other neurotransmitters.

In another embodiment, the compound is 1-oxa-8-thia-3-aza-dibenzo[e,h]azulene; 1,8-dioxa-3-aza-dibenzo[e,h]azulene; 3-(1-oxa-8-thia-3-aza-dibenzo[e,h]azulen-2-yl)-propionic acid ethyl ester; 3-(1,8-dioxa-3-aza-dibenzo[e,h]azulen-2-yl)-propionic acid ethyl ester; 2-methyl-1-oxa-8-thia-3-aza-dibenzo[e,h]azulene; 2-methyl-1,8-dioxa-3-aza-dibenzo[e,h]azulene; 1 1-chloro-2-methyl-1-oxa-8-thia-3-aza-dibenzo[e,h]azulene; 5-chloro-2-methyl-1-oxa-8-thia-3-aza-dibenzo[e,h]azulene; 11-chloro-2-methyl-1,8-dioxa-3-aza-dibenzo[e,h]azulene; 5-chloro-2-methyl-1,8-dioxa-3-aza-dibenzo[e,h]azulene; 1-oxa-8-thia-3-aza-dibenzo[e,h]azulene-2-carbaldehyde; 3-(1-oxa-8-thia-3-aza-dibenzo[e,h]azulen-2-yl)-propionic acid; 3-(1,8-dioxa-3-aza-dibenzo[e,h]azulen-2-yl)-propionic acid; (1-oxa-8-thia-3-aza-dibenzo[e,h]azulen-2-yl)-methanol; 3-(1-oxa-8-thia-3-aza-dibenzo[e,h]azulen-2-yl)-propane-1-ol; 3-(1,8-dioxa-3-aza-dibenzo[e,h]azulen-2-yl)-propane-1-ol; 2-bromomethyl-1-oxa-8-thia-3-aza-dibenzo[e,h]azulene; 2-bromomethyl-1,8-dioxa-3-aza-dibenzo[e,h]azulene; 2-bromomethyl-5-chloro-1-oxa-8-thia-3-aza-dibenzo[e,h]azulene; 2-bromomethyl-11-chloro-1-oxa-8-thia-3-aza-dibenzo[e,h]azulene; 2-bromomethyl-5-chloro-1,8-dioxa-3-aza-dibenzo[e,h]azulene; 2-bromomethyl-11-chloro-1,8-dioxa-3-aza-dibenzo[e,h]azulene; dimethyl-[2-(1-oxa-8-thia-3-aza-dibenzo[e,h]azulen-2-ylmethoxy)-ethyl]-am-ine; dimethyl-[3-(1-oxa-8-thia-3-aza-dibenzo[e,h]azulen-2-ylmethoxy)-propyl]-a-mine; dimethyl-{2-[3-(1-oxa-8-thia-3-aza-dibenzo[e,h]azulen-2-yl)-propoxy]-ethy-1}-amine; dimethyl-{3-[3-(1-oxa-8-thia-3-aza-dibenzo[e,h]azulen-2-yl)-propoxy]-prop-yl}-amine; {2-[3-(1,8-dioxa-3-aza-dibenzo[e,h]azulen-2-yl)-propoxy]-ethyl}-dimethyla-mine; {3-[3-(1,8-dioxa-3-aza-dibenzo[e,h]azulen-2-yl)-propoxy]-propyl}-dimethyl-amine; [2-(1,8-dioxa-3-aza-dibenzo[e,h]azulen-2-ylmethoxy)-ethyl]-dimethylamine; [3-(1,8-dioxa-3-aza-dibenzo[e,h]azulen-2-ylmethoxy)-propyl]-dimet-hylamine; 2-(5-chloro-1-oxa-8-thia-3-aza-dibenzo[e,h]azulen-2-ylmethoxy)-ethyl]-dim-ethylamine; [3-(5-chloro-1-oxa-8-thia-3-aza-dibenzo[e,h]azulen-2-ylmethoxy)-propyl]-d-imethylamine; [2-(1 1-chloro-1-oxa-8-thia-3-aza-dibenzo[e,h]azulen-2-ylmethoxy)-ethyl]-d-imethylamine; [3-(11-chloro-1-oxa-8-thia-3-aza-dibenzo[e,h]azulen-2-ylmethoxy)-propyl]-dimethylamine; [2-(5-chloro-1,8-dioxa-3-aza-dibenzo[e,h]azulen-2-ylmethoxy)-ethyl]-dimet-hylamine; [3-(5-chloro-1,8-dioxa-3-aza-dibenzo[e,h]azulen-2-ylmethoxy)-propyl]-dime-thylamine; [2-(1 1-chloro-1,8-dioxa-3-aza-dibenzo[e,h]azulen-2-ylmethoxy)-ethyl]-dime-thylamine; or [3-(11-chloro-1,8-dioxa-3-aza-dibenzo[e,h]azulen-2-ylmethoxy)-propyl]-dim-ethylamine, including pharmaceutically-acceptable salts and derivatives thereof.

### Formula 214

In an embodiment, the NMDA receptor antagonist may be selected from one of the following compounds according to Formula 214. Such compounds are described in US 2010/0130617, published May 27, 2010 and corresponding to U.S. application Ser. No. 12/623,948, filed Nov. 23, 2009, which are each incorporated by reference in their entirety herein. In the case of any conflict of terminology of Formula 214, these references incorporated by reference herein control.

In an embodiment, the NMDA receptor antagonist is a compound according to Formula 214:

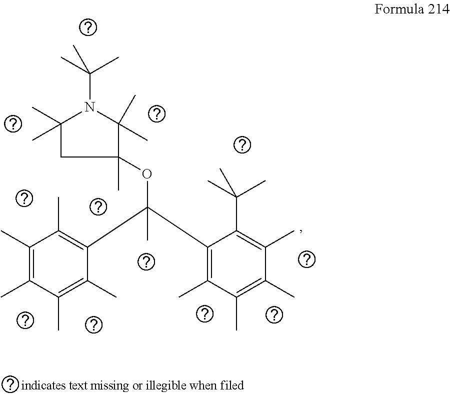

or a salt thereof, wherein:
R1-R23 are independently selected from the group consisting of hydrogen and deuterium;
at least one of R1-R23 is deuterium; and
   if each of R1-R3 are deuterium, then at least one of R4-R23 is deuterium.

In further embodiments, the NMDA receptor antagonist is a compound according to:

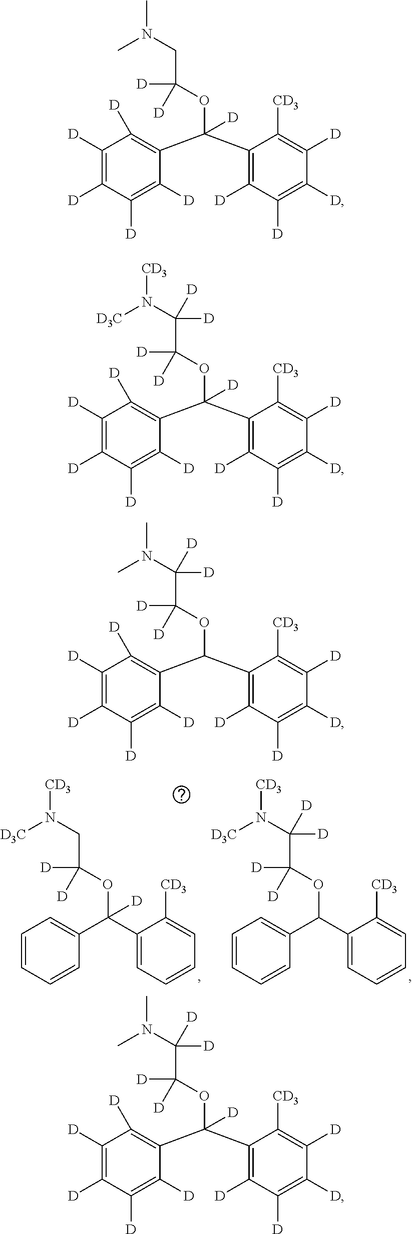

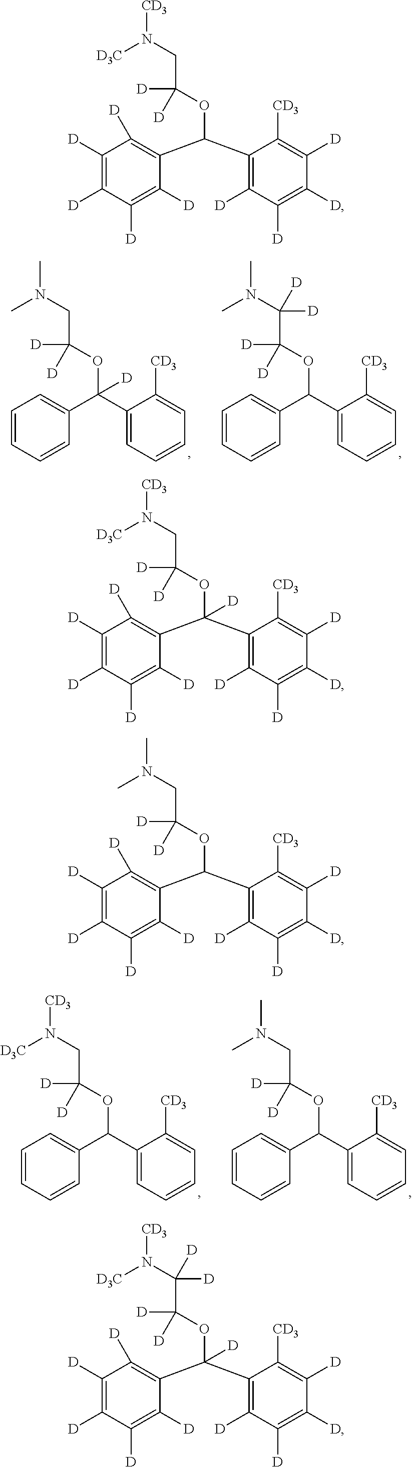

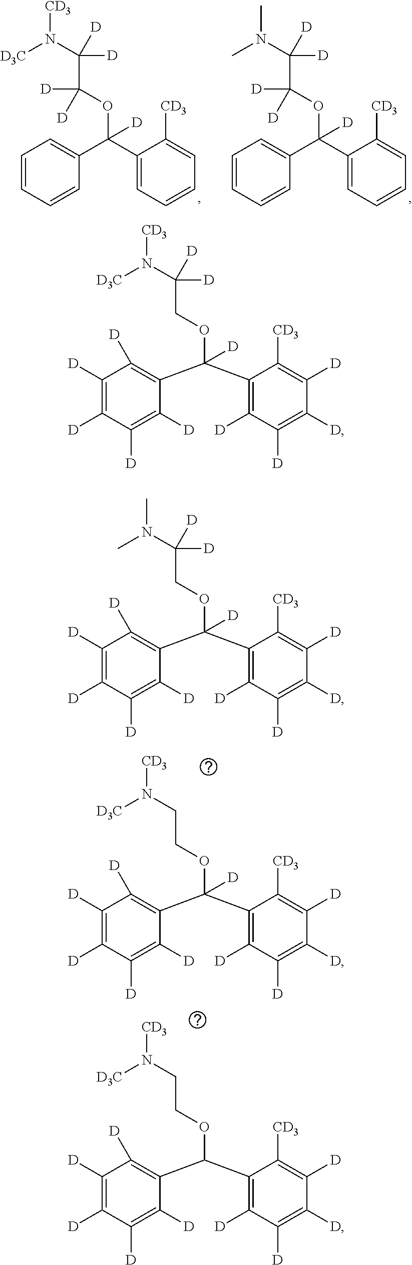

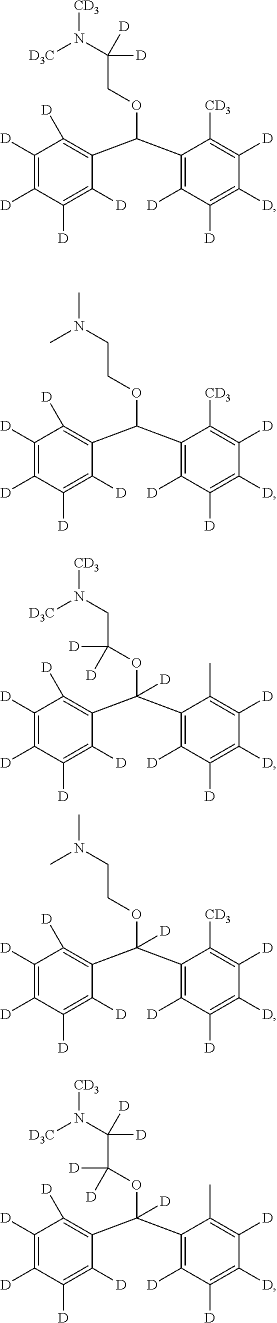

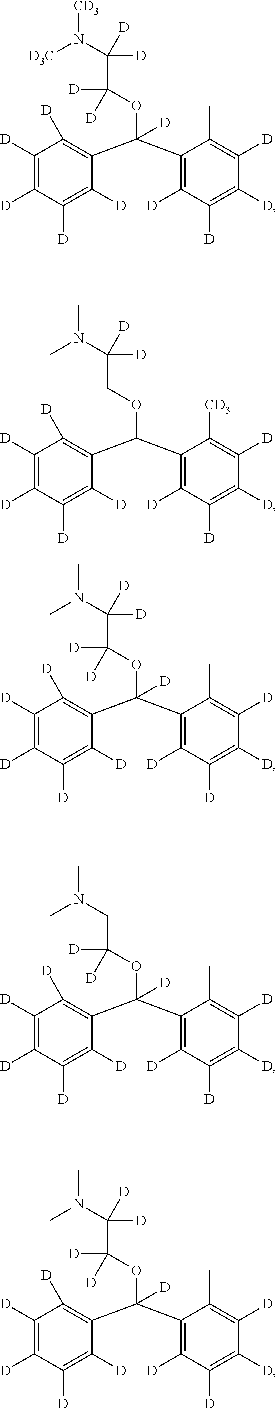

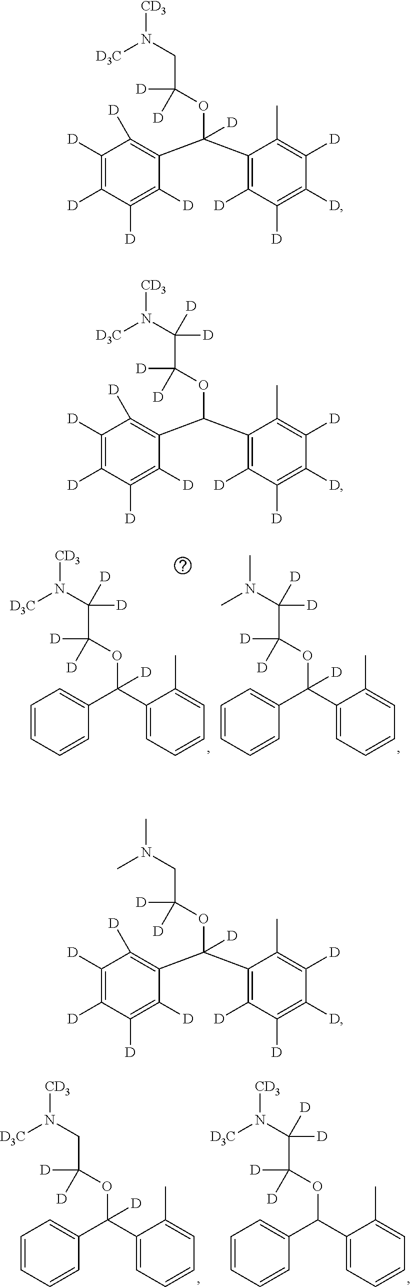

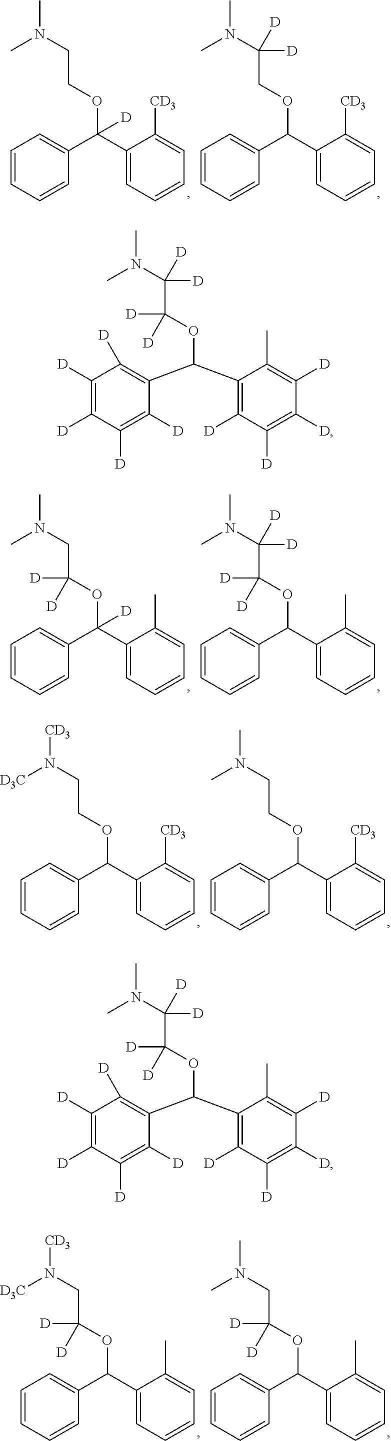

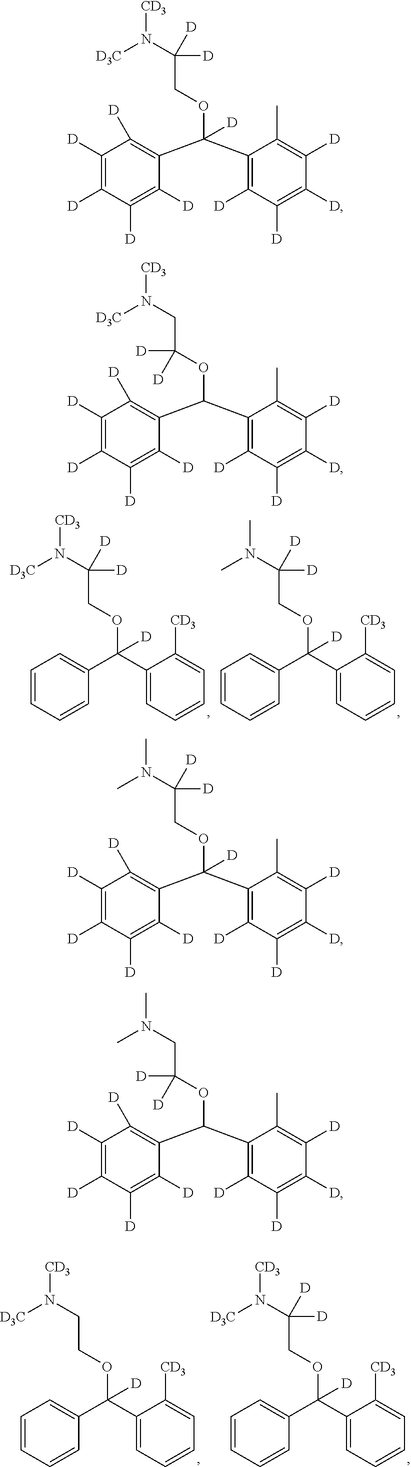

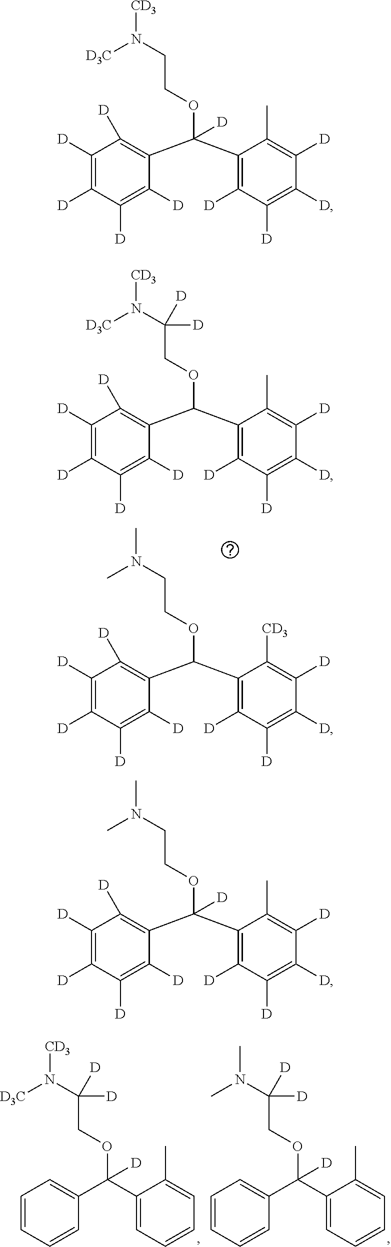

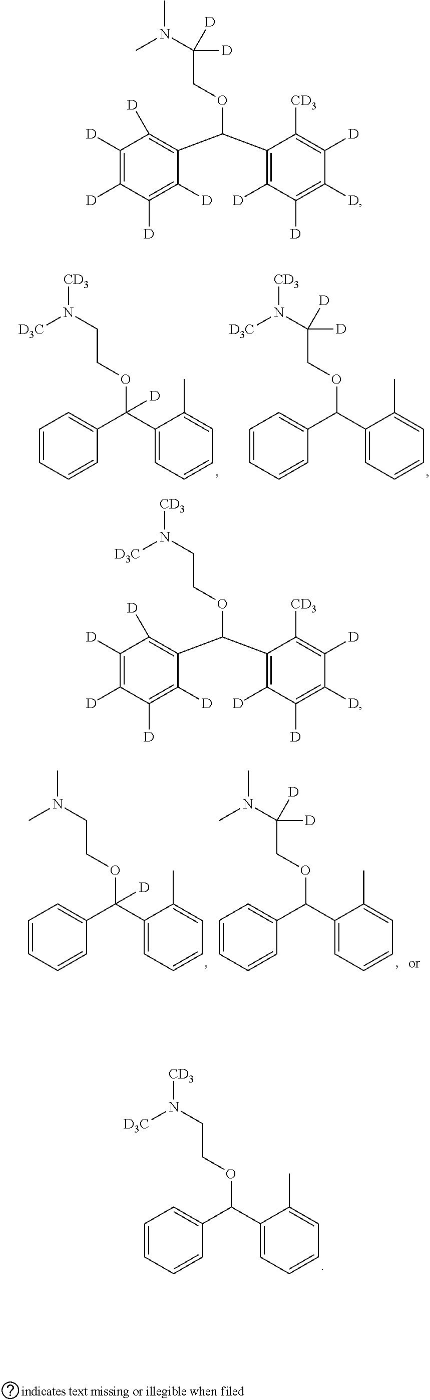

In an embodiment, the NMDA receptor antagonist may be selected from one of the following compounds according to Formula 215. Such compounds are described in WO 2010/0033757, published Mar. 25, 2010 and corresponding to PCT/US2009/057401, filed Sep. 18, 2009, U.S. Pat. No. 9,512,133, issued Dec. 6, 2016, U.S. Pat. No. 9,802,946, issued Oct. 31, 2017, and U.S. Pat. No. 10,906,913, issued Feb. 2, 2021, which are each incorporated by reference in their entirety herein. In the case of any conflict of terminology of Formula 215, these references incorporated by reference herein control.

In an embodiment, the NMDA receptor antagonist is a compound according to:

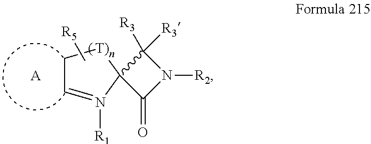

and pharmaceutically acceptable salts, stereoisomers, and N-oxides thereof; wherein
T is, independently for each occurrence, CR4R4, and n is 0, 1, 2 or 3; A is optionally present and is selected from phenyl or pyridine, wherein A is optionally substituted by one or more substituents selected from Ra; R1 is selected from the group consisting of H, hydroxyl, —S(O)2-C1-C4alkyl; —SO2, C1-C4alkyl, C2-C4alkenyl, phenyl, R7, or

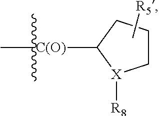

wherein C1-C4alkyl, C2-C4alkenyl, or phenyl is optionally substituted by one or more substituents selected from Ra; X is CH or N; R3 and R3 are independently selected from the group consisting of H, halogen, hydroxyl, phenyl, C1-C4alkyl, amido, amine, or C2-C4alkenyl, wherein C1-C4alkyl, C2-C4alkenyl and phenyl are optionally substituted by one or more substituents selected from Ra; R4 and R4 are independently selected from the group consisting of H, halogen, hydroxyl, phenyl, C1-C4alkyl, amido, amine, CrC4alkoxy or C2-C4alkenyl, wherein C1-C4alkyl, C2-C4alkenyl, C1-C4alkoxy, and phenyl are optionally substituted by one or more substituents selected from Ra; R2 is selected from the group consisting of H, R7, —S(O)2, S(O)2-C1-C4alkyl, Q-Qalkyl, hydroxyl, or phenyl wherein C1-C4lkyl, C2-C4 alkenyl and phenyl are optionally substituted by one or more substituents selected from Ra; R5 and R5′ are each independently selected from group consisting of H, halogen, C1-C4alkyl, C1-C4alkoxy, C2-C4alkenyl, cyano, amino, phenyl, and hydroxyl, wherein C1-C4alkyl, C2-C4alkenyl and phenyl are optionally substituted by one or more substituents selected from Ra; R7 is selected from group consisting of —C(O)—C1-C4alkyl or C(O)—O—C1-C4alkyl, wherein C1-C4 alkyl is optionally substituted by 1, 2 or 3 substituents selected from Rb; R8 is selected from group consisting of H, —C(O)—C1-C4 alkyl or C(O)—O—C1-C4 alkyl, wherein C1-C4alkyl is optionally substituted by 1, 2 or 3 substituents selected from Ra; Ra is selected, independently for each occurrence, from carboxy, hydroxyl, halogen, amino, phenyl, C1-C4 alkyl, and C1-C4 alkoxy; Rb is selected, independently for each occurrence, from the group consisting of carboxy, hydroxyl, halogen, amino, phenyl, C1-C4 alkyl, C1-C4 alkoxy, and —NH—RC; and Rc is selected, independently for each occurrence, —C(O)—O—C1-C4alkyl; and —C(O)—C1-C4alkyl.

In further embodiments, the NMDA receptor antagonist is a compound according to:

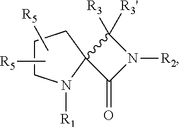

wherein R1 is C(O)—C2-C4alkyl, wherein C2-C4alkyl is substituted at one carbon with NH2 or —N-carbobenzyloxy and at a different carbon by hydroxyl.

In further embodiments, the NMDA receptor antagonist is represented by:

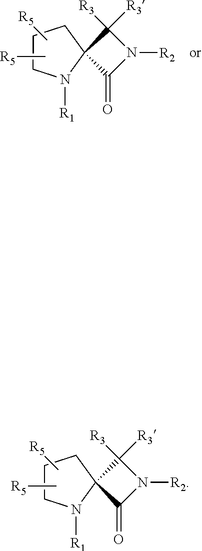

In further embodiments, the NMDA receptor antagonist is a compound according to:

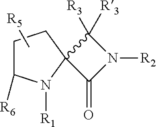

and pharmaceutically acceptable salts, stereoisomers and N-oxides thereof; wherein R1 is selected from the group consisting of H, hydroxyl, —S(O)2-C1-C4alkyl; —SO2, C1-C 4alkyl; R7, or

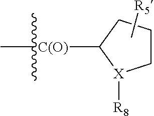

X is CH or N; R3 and R3′ are each independently selected from the group consisting of H, halogen, hydroxyl, phenyl, C1-C4alkyl, amido, amine, or C2-C4alkenyl, wherein C1-C4alkyl, C2-C4alkenyl and phenyl are optionally substituted by one or more substituents selected from Ra; R2 is selected from the group consisting of H, R7, —S(O)2, S(O)2-C1-C4alkyl, C1-C4 alkyl, hydroxyl, or phenyl wherein C1-C4alkyl, C2-C4 alkenyl and phenyl are optionally substituted by one or more substituents selected from Ra; R5 is selected from group consisting of H, halogen, C1-C4 alkyl, C1-C4 alkoxy, C2-C4 alkenyl, cyano, amino, phenyl, and hydroxyl, wherein C1-C4alkyl, C2-C4alkenyl and phenyl are optionally substituted by one or more substituents selected from Ra; Re is selected from group consisting of H, halogen, C1-C4 alkyl, C1-C4 alkoxy, C2-C4alkenyl, cyano, amino, phenyl, and hydroxyl wherein C1-C4 alkyl, C2-C4alkenyl and phenyl are optionally substituted by 1, 2 or 3 substituents selected from Ra; R7 is selected from group consisting of —C(O)—C1-C4alkyl or —C(O)—O—C1-C4alkyl, wherein C1-C4 alkyl is optionally substituted by 1, 2 or 3 substituents selected from Rb; or or R1 and R6, taken together with formula II form:

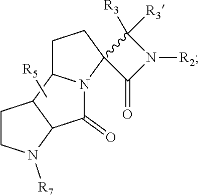

R8 is selected from group consisting of H, —C(O)—C1-C4alkyl or C(O)—O—C1-C4 alkyl, wherein C1-C4alkyl is optionally substituted by 1, 2 or 3 substituents selected from Ra; Ra is selected, independently for each occurrence, from carboxy, hydroxyl, halogen, amino, phenyl, C1-C4alkyl, and C1-C4alkoxy;
Rb is selected, independently for each occurrence, from the group consisting of carboxy, hydroxyl, halogen, amino, phenyl, C1-C4alkyl, C1-C4alkoxy, and —NH—RC; and
Rc is selected, independently for each occurrence, —C(O)—O—C1-C4alkyl; and —C(O)—C1-C4alkyl.

In further embodiments, the NMDA receptor antagonist is a compound selected from:

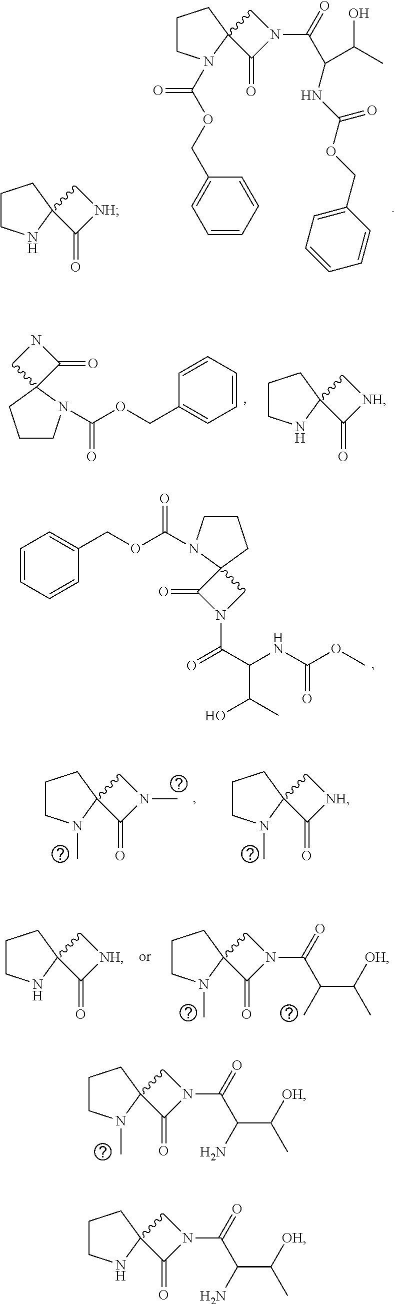

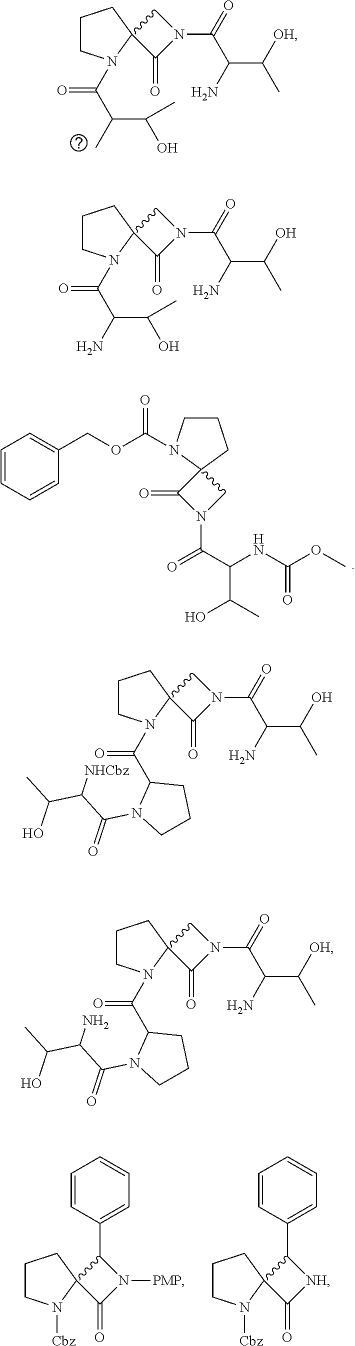

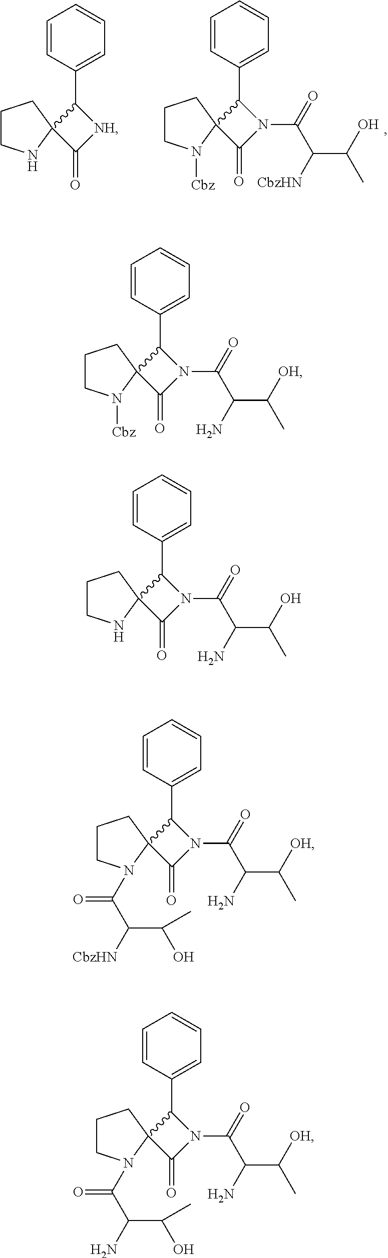

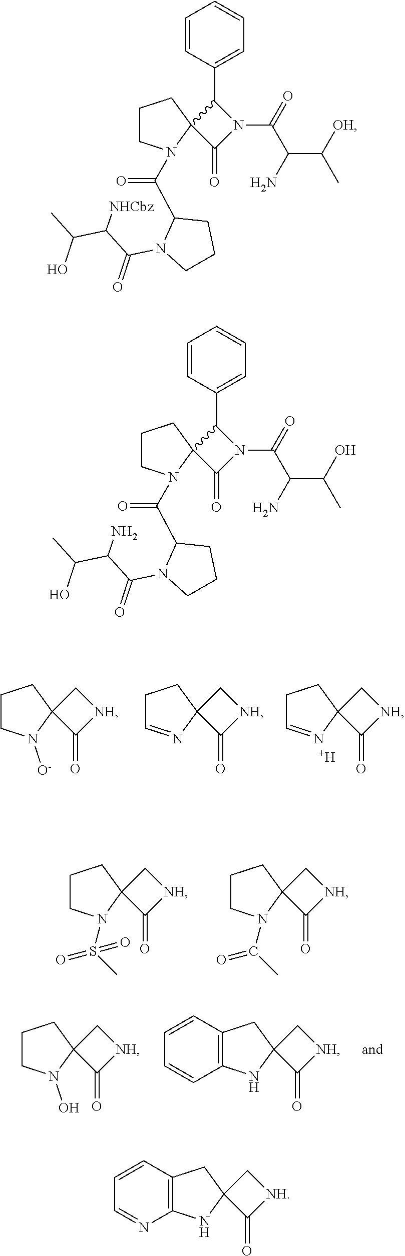

In an embodiment, the NMDA receptor antagonist may be selected from one of the following compounds according to Formula 216. Such compounds are described in US 2009/0004112, published Jan. 1, 2009 and corresponding to application Ser. No. 12/049,782, filed Mar. 17, 2008, which are each incorporated by reference in their entirety herein. In the case of any conflict of terminology of Formula 216, these references incorporated by reference herein control.

In an embodiment, the NMDA receptor antagonist is a compound according to Formula 216:

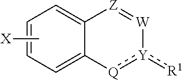

or a pharmaceutically acceptable base or acid addition salt, hydrate, stereoisomer, or mixture thereof, wherein
X is one or more halogen radicals;
Q is NH or N;
W is CR2, CHR2, NR3, or CH═COH;
Y is C or CH;
Z is C═O, SO2, COH, CHOH, or NHR4;
R1 is CO2H or oxo (═O);
R2 is H, C(═O)—C1-C6 alkyl, C(═O)O—C1-C6 alkyl, or C(═O)—C3-C8 cycloalkyl;
R3 is optionally substituted C3-C10 aryl; and
R4 is

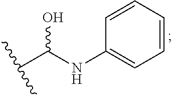 [SMILES: *C(O)Nc1ccccc1]

or
R2 and R1 combine with the carbons to which they are attached to form a 6 membered heterocycle that is optionally substituted at one or more of the heteroatoms with C3-C10 aryl, wherein the aryl may be substituted with one or more of C1-C6 alkyl or —O—C1-C6 alkyl, and the heteroatoms in the heterocyclic ring are one or more nitrogen atoms.

In further embodiments, the NMDA receptor antagonist is a compound according to:

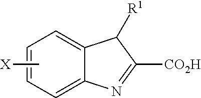

or a pharmaceutically acceptable base or acid addition salt, hydrate, stereoisomer, or mixture thereof, wherein
X is one or more halogen radicals;
R1 is (CH2)n-CO2H, or CH═CHC(═O) NHR2; and
R2 is C3-C10 aryl optionally substituted with one or more of C1-C6 alkyl, —O—C1-C6 alkyl, or halogen; and
   n is 0, 1, 2, 3, 4, 5, or 6.

In an embodiment, the NMDA receptor antagonist is an NMDA receptor glycine site antagonist.

In an embodiment of the present invention, the NMDA receptor antagonist is ACEA 1021 (liconstinel), GV150526 (Gavestinal), GV196771, MDL 105,519, L-701324, L-687414, RPR 104632, ACPC (SYM2030), ZD9379, AR-R15896AR, or RPR118723.

In an embodiment, the NMDA receptor antagonist is selected from:

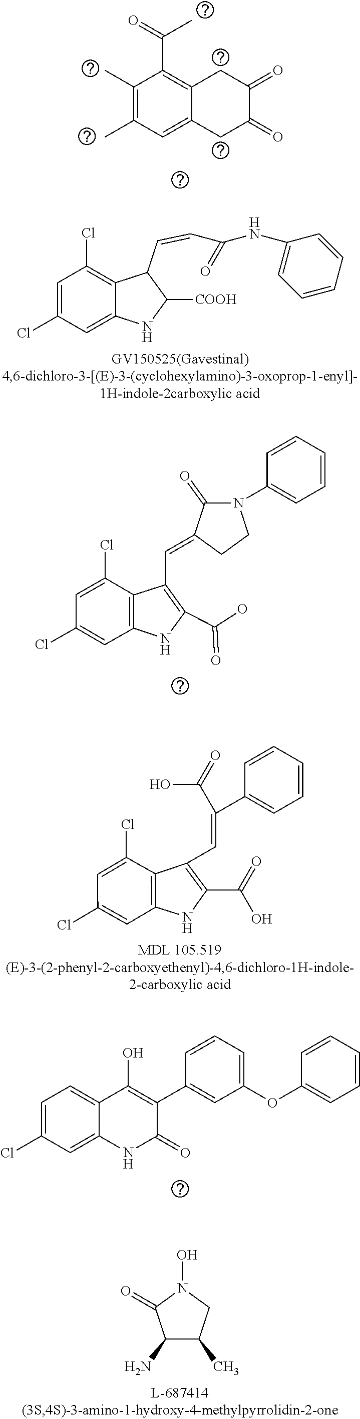

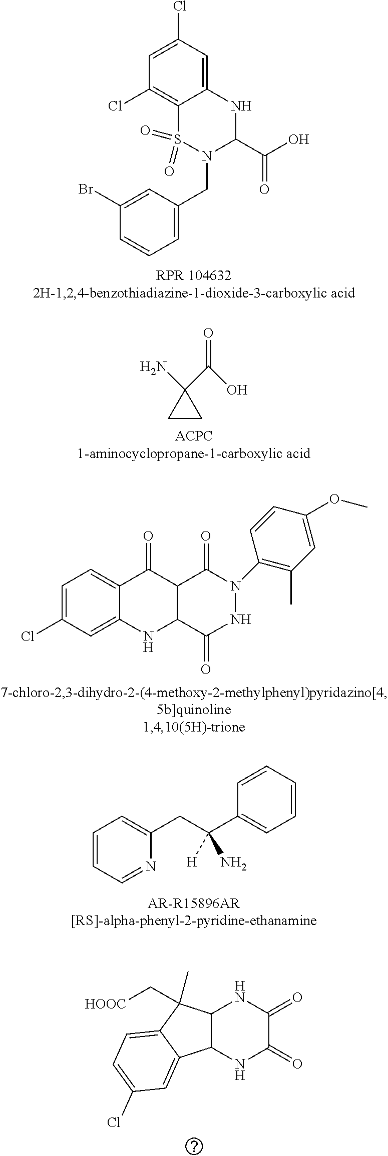 [SMILES: O=C(O)C1Nc2cc(Cl)cc(Cl)c2S(=O)(=O)N1Cc1cccc(Br)c1 ; NC1(C(=O)O)CC1 ; COc1ccc(N2NC(=O)C3Nc4cc(Cl)ccc4C(=O)C3C2=O)c(C)c1 ; N[C@@H](Cc1ccccn1)c1ccccc1 ; CC1(CC(=O)O)c2ccc(Cl)cc2C2NC(=O)C(=O)NC21]

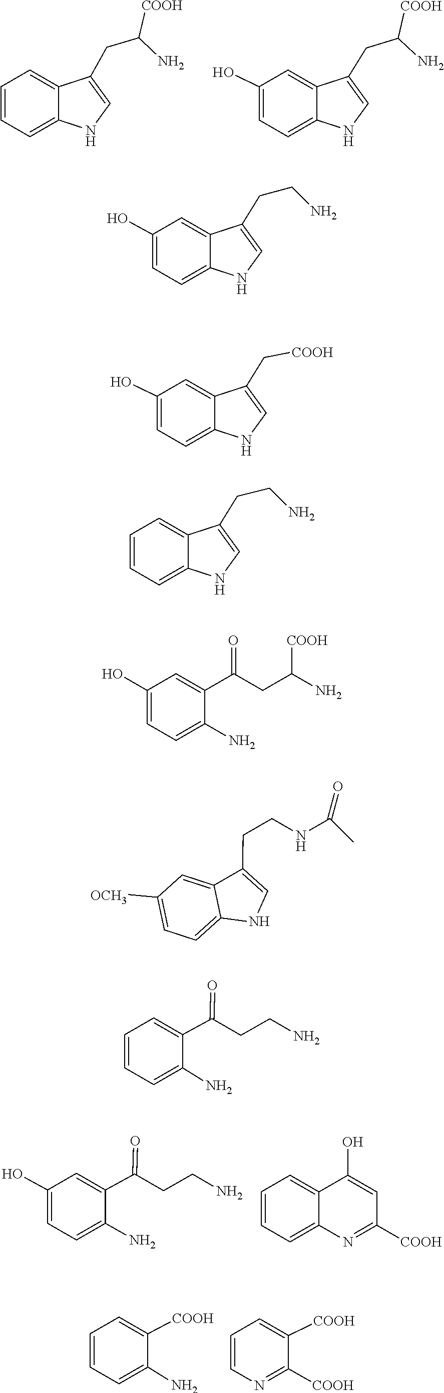

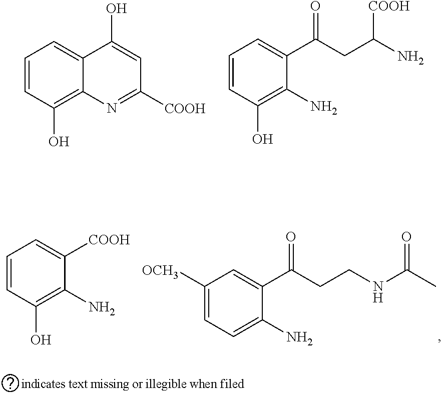 [SMILES: O=C(O)c1cc(O)c2cccc(O)c2n1 ; Nc1c(O)cccc1C(=O)CC(N)C(=O)O ; Nc1c(O)cccc1C(=O)O ; COc1ccc(N)c(C(=O)CCNC(C)=O)c1]

or a pharmaceutically acceptable salt thereof.

### Formula 217

In an embodiment, the NMDA receptor antagonist may be selected from one of the following compounds according to Formula 217. Such compounds are described in WO2008/134525, published Nov. 6, 2008 and corresponding to PCT/US2008/061598, filed Apr. 25, 2008, which are each incorporated by reference in their entirety herein. In the case of any conflict of terminology of Formula 217, these references incorporated by reference herein control.

In an embodiment, the NMDA receptor antagonist is a deuterium-labeled ketamine analogue.

In an embodiment, the NMDA receptor antagonist is a compound according to Formula 217:

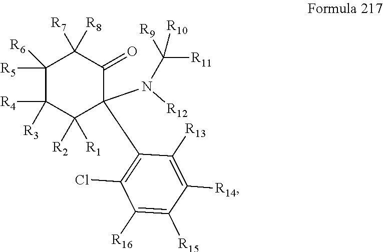

or a pharmaceutically
acceptable salt, solvate, or prodrug thereof, wherein:
R1, R2, R3, R4, R5, R6, R7, R8, R9, R10, R11, R12, R13, R14, R15, and R16 are independently selected from the group consisting of hydrogen and deuterium; and at least one of R1, R2, R3, R4, R5, R6, R7, R8, R9, R10, R11, R12, R13, R14, R15, and R16 is deuterium.

In further embodiments, the NMDA receptor antagonist is a compound selected from:

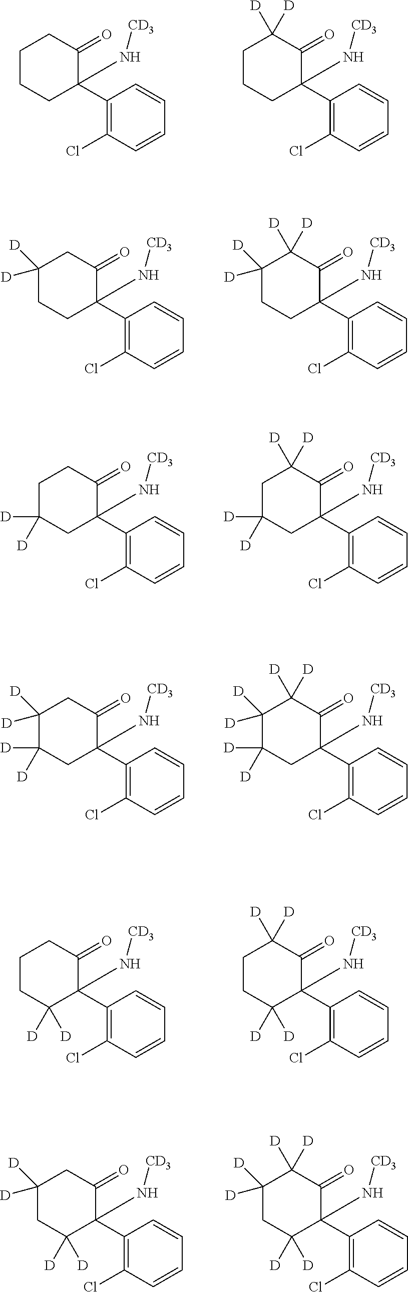

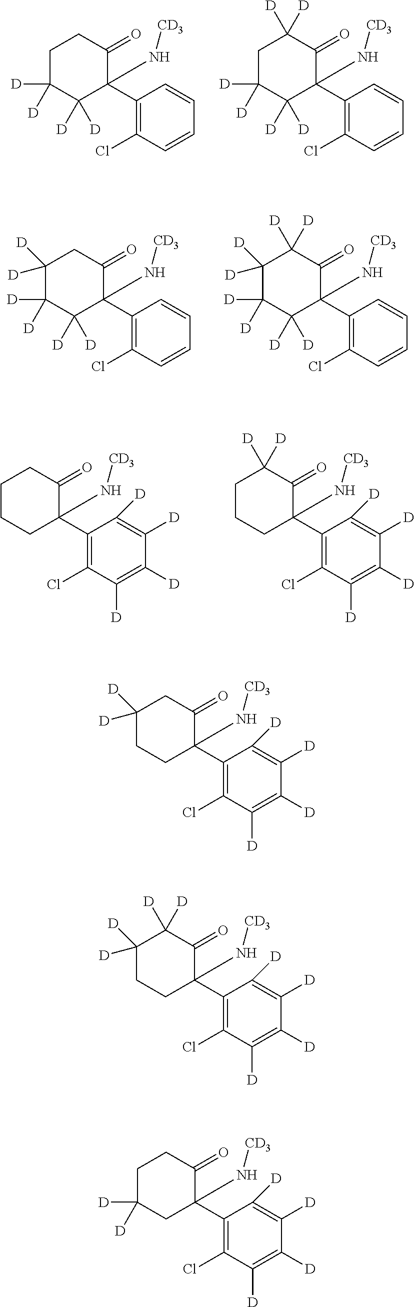

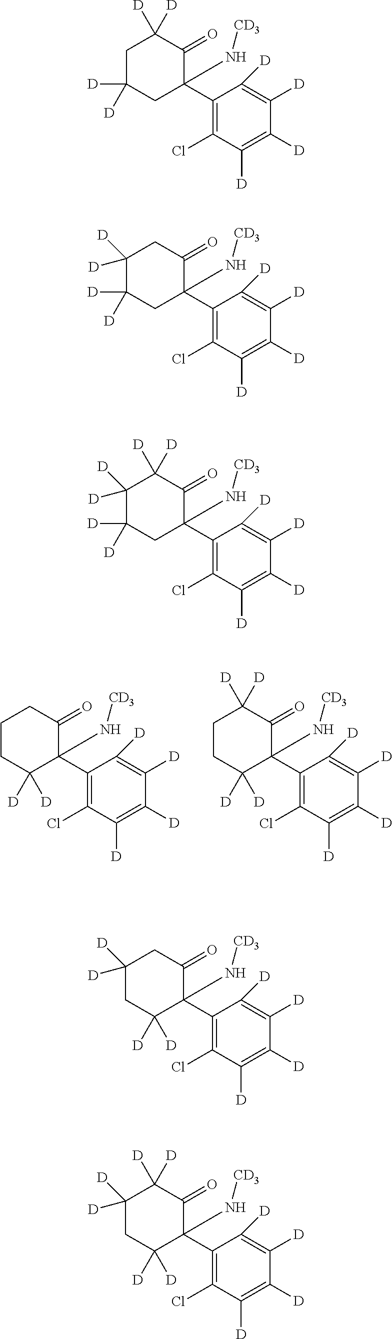

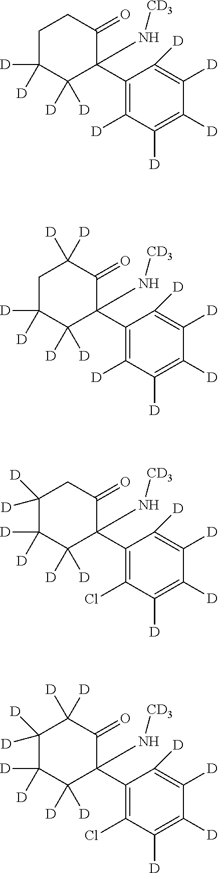 [SMILES: [2H]c1c([2H])c([2H])c(C2(NC([2H])([2H])[2H])C(=O)CCC([2H])([2H])C2([2H])[2H])c([2H])c1[2H] ; [2H]c1c([2H])c([2H])c(C2(NC([2H])([2H])[2H])C(=O)C([2H])([2H])CC([2H])([2H])C2([2H])[2H])c([2H])c1[2H] ; [2H]c1c([2H])c([2H])c(C2(NC([2H])([2H])[2H])C(=O)CC([2H])([2H])C([2H])([2H])C2([2H])[2H])c(Cl)c1[2H] ; [2H]c1c([2H])c([2H])c(C2(NC([2H])([2H])[2H])C(=O)C([2H])([2H])C([2H])([2H])C([2H])([2H])C2([2H])[2H])c(Cl)c1[2H]]

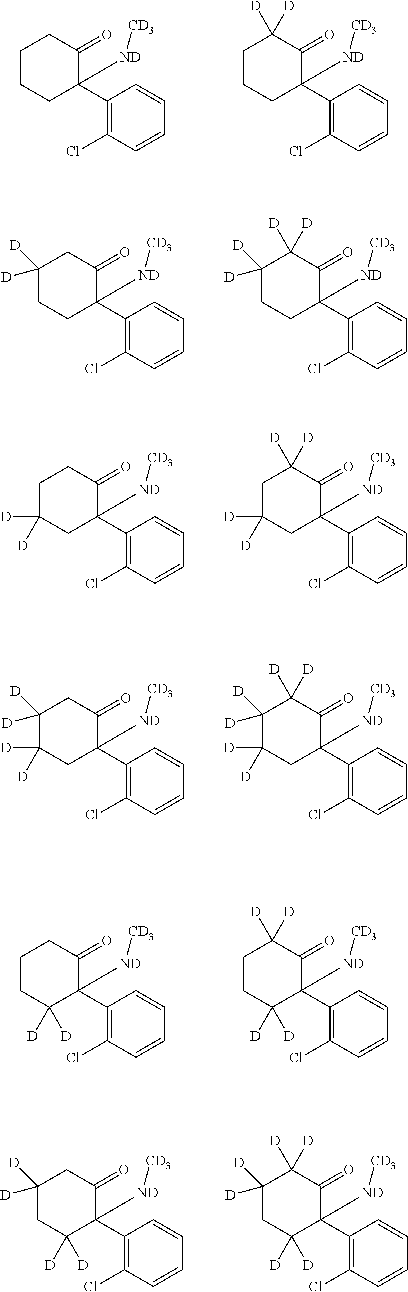

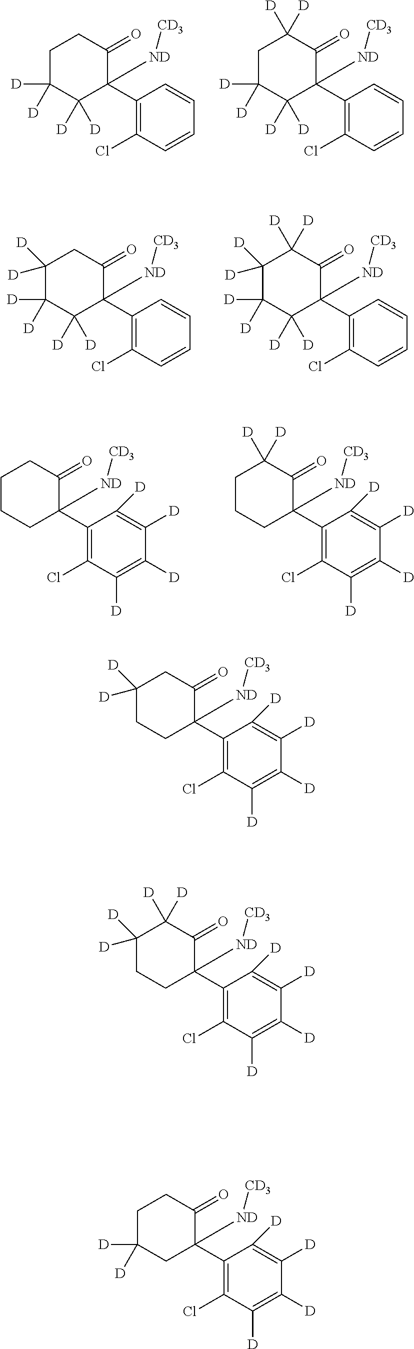

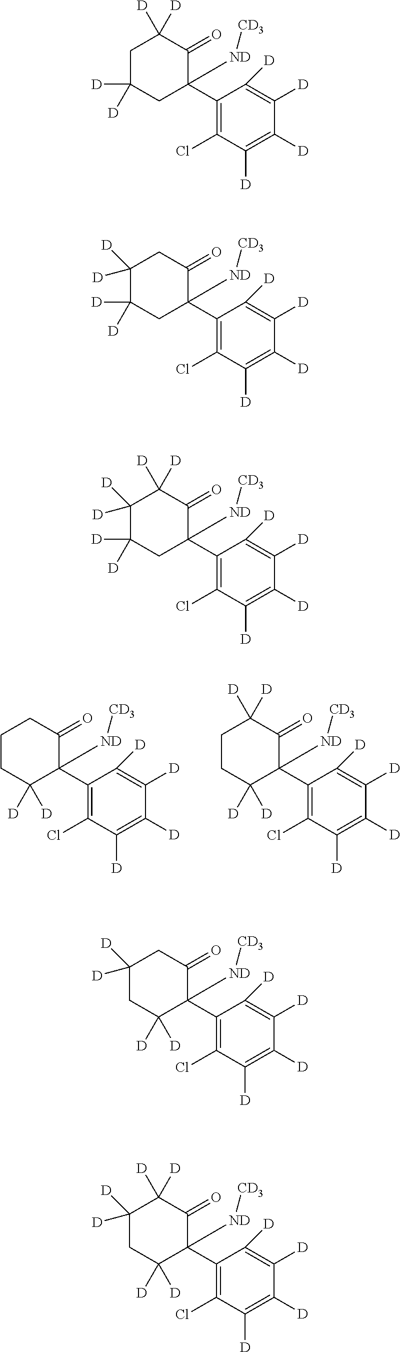

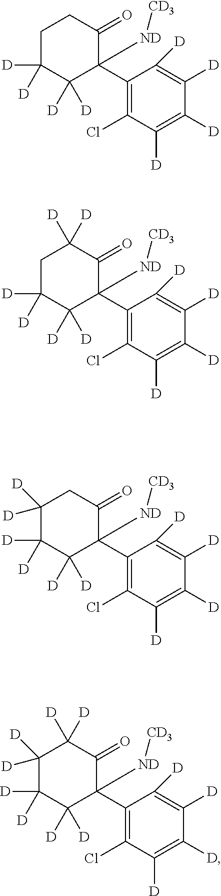

or a pharmaceutically acceptable salt, solvate or prodrug thereof.

In an embodiment, the NMDA receptor antagonist may be selected from one of the following compounds according to Formula 218. Such compounds are described in WO 2008/022285, published February 2008, corresponding to PCT/US2007/076146, filed Aug. 16, 2007 which are each incorporated by reference in their entirety herein. In the case of any conflict of terminology of Formula 218, these references incorporated by reference herein control.

In an embodiment, the NMDA receptor antagonist is a compound according to Formula 218:

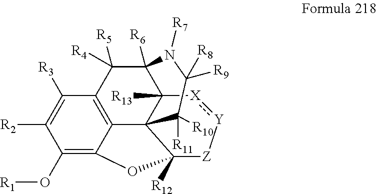

a single enantiomer, a mixture of a (+)-enantiomer and a (−)-enantiomer, a mixture of about 90% or more by weight of the (−)-enantiomer and about 10% or less by weight of the (+)-enantiomer, a mixture of about 90% or more by weight of the (+)-enantiomer and about 10% or less by weight of the (−)-enantiomer, an individual diastereomer, or a mixture of diastereomers thereof, or a pharmaceutically acceptable salt, solvate, or prodrug thereof, wherein: any bond represented by a dashed line and a solid line represents a bond selected from the group consisting of a single bond and a double bond, and any carbon-carbon double bond has a configuration selected from the group consisting of cis and trans; R2, R3, R4, R5, R6, R8, R9, R10, R11 and R12 are independently selected from the group consisting of hydrogen, and deuterium;
R1 and R7 are independently selected from the group consisting of hydrogen, deuterium, —CH3, —CDH2, —CD2H, and —CD3;
R13 is selected from the group consisting of hydrogen, deuterium, —OH, and —OD;
when the bond represented by a dashed line and a solid line represents a double bond, X and Y are independently selected from the group consisting of C—H, and C-D; when the bond represented by a dashed line and a solid line represents a single bond, X and
Y are independently selected from the group consisting of CH2, CHD, and CD2;
   Z is selected from the group consisting of C(H) OH, C(H) OD, C (D) OH, C (D) OD, and carbonyl (C═O); provided that the compound contains at least one deuterium atom and that deuterium enrichment in the compound is at least about 1%.

In an embodiment, the NMDA receptor antagonist is a compound according to:

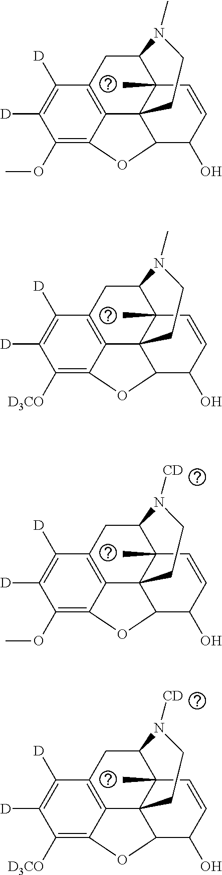

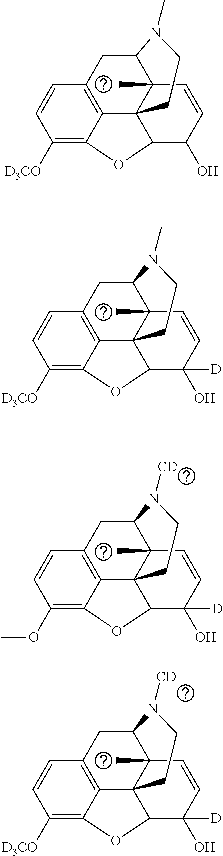

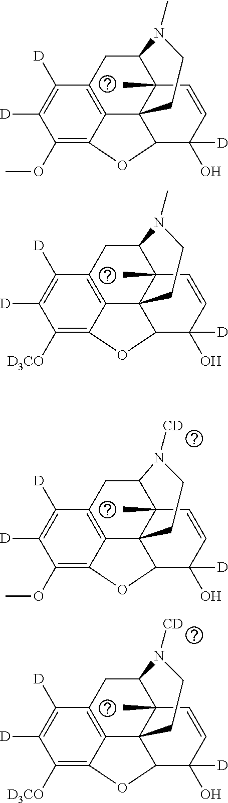

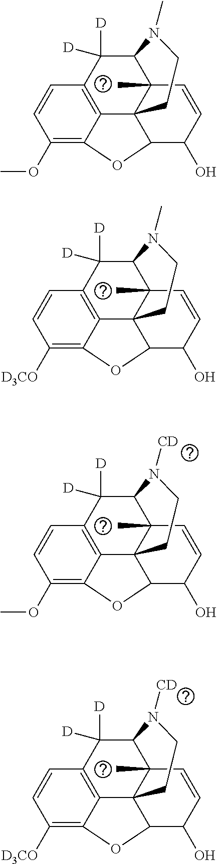

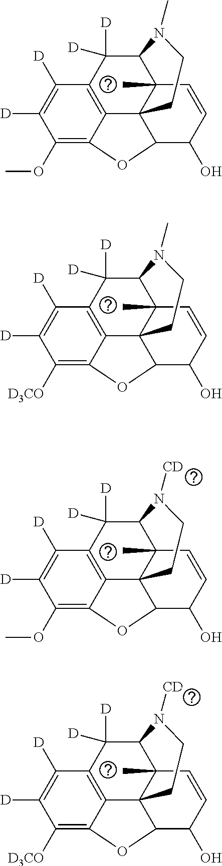

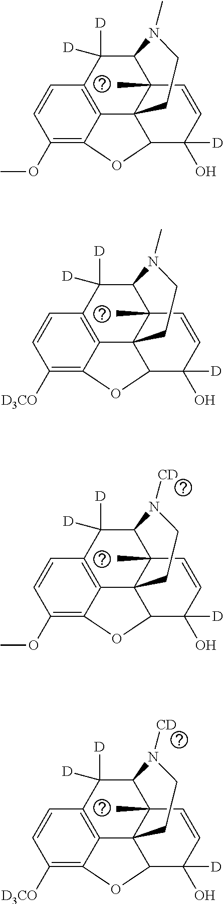

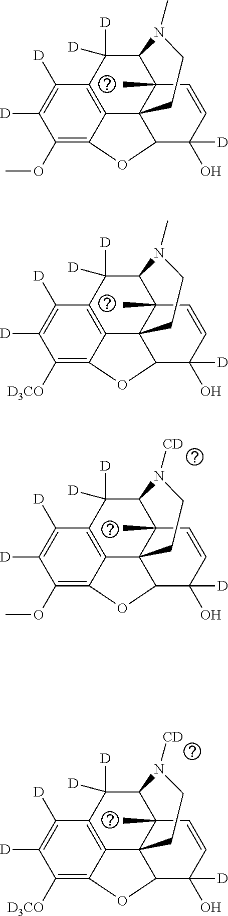

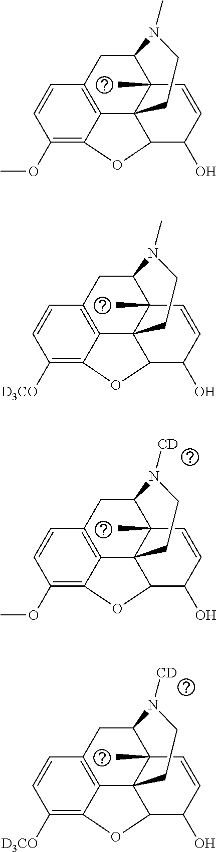

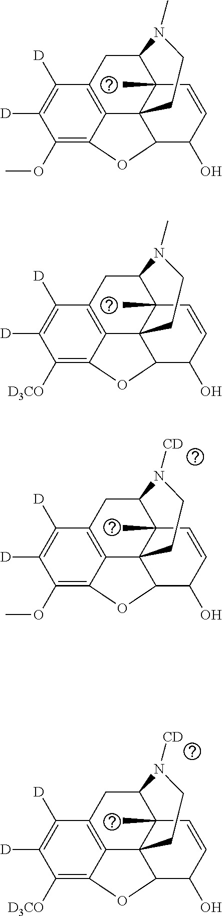

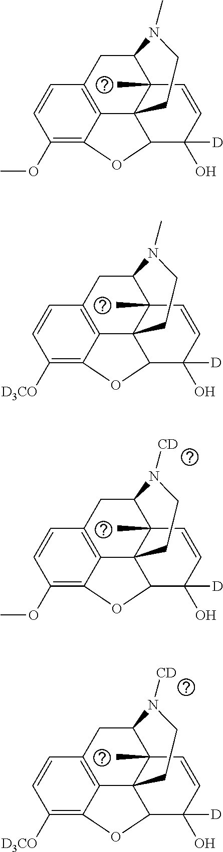

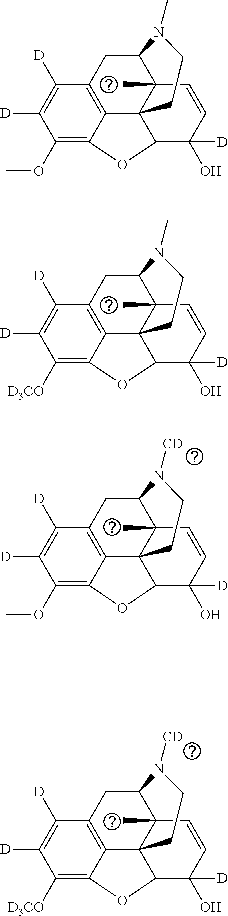

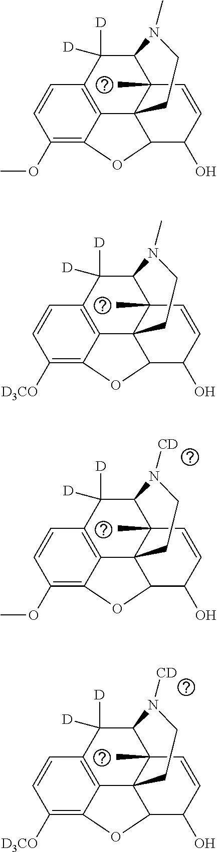

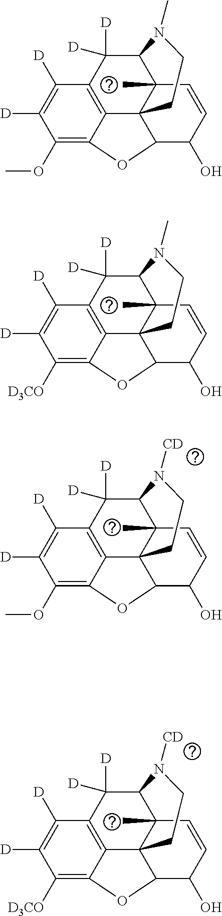

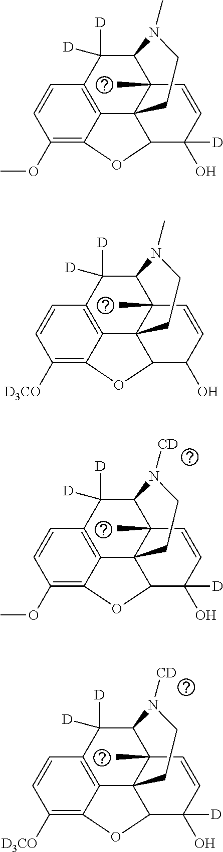

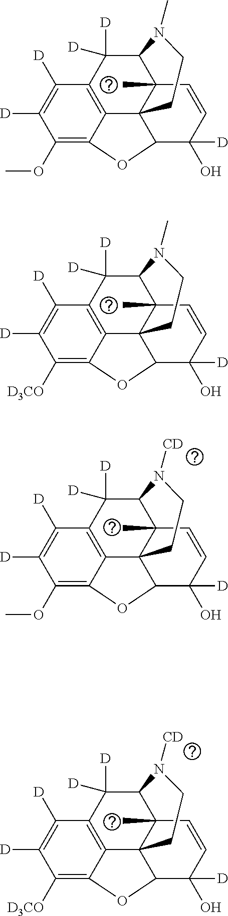

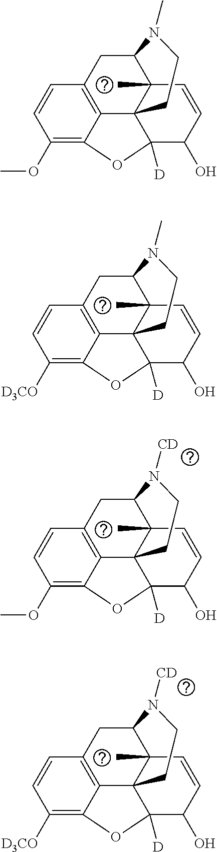

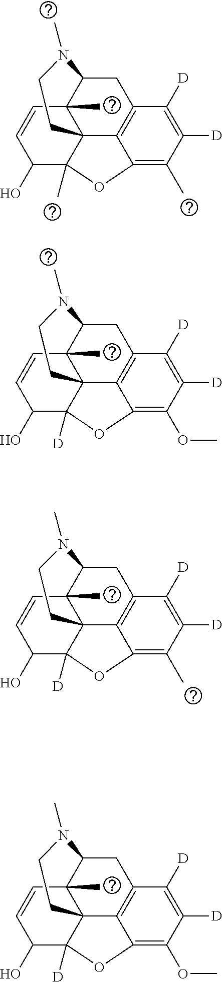 [SMILES: [2H]c1c([2H])c2c3c(c1C)OC1(C)C(O)C=C[C@]4(C)[C@H](C2)N(C)CC[C@@]314 ; [2H]c1c([2H])c(OC)c2c3c1C[C@@H]1N(C)CC[C@]34C([2H])(O2)C(O)C=C[C@]14C ; [2H]c1c([2H])c2c3c(c1C)OC1([2H])C(O)C=C[C@]4(C)[C@H](C2)N(C)CC[C@@]314 ; [2H]c1c([2H])c(OC)c2c3c1C[C@@H]1N(C)CC[C@]34C([2H])(O2)C(O)C=C[C@]14C]

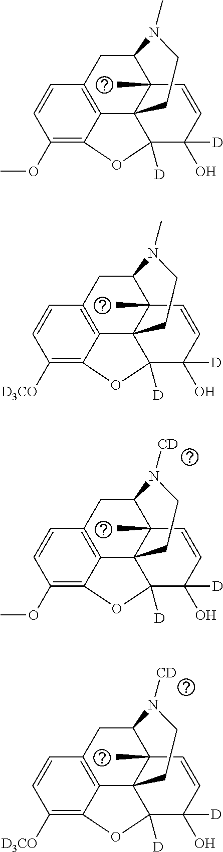

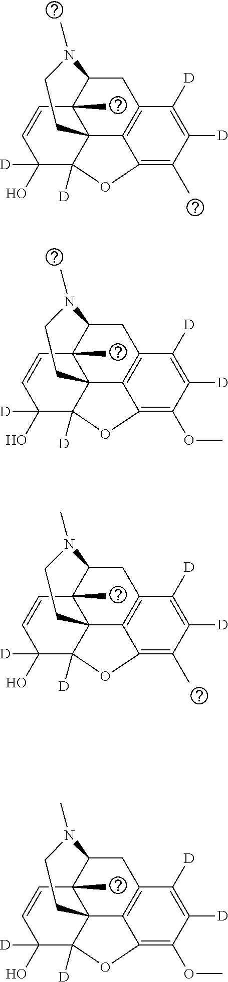 [SMILES: [2H]c1c([2H])c2c3c(c1C)OC1([2H])C([2H])(O)C=C[C@]4(C)[C@H](C2)N(C)CC[C@@]314 ; [2H]c1c([2H])c(OC)c2c3c1C[C@@H]1N(C)CC[C@]34C([2H])(O2)C([2H])(O)C=C[C@]14C ; [2H]c1c([2H])c2c3c(c1C)OC1([2H])C([2H])(O)C=C[C@]4(C)[C@H](C2)N(C)CC[C@@]314 ; [2H]c1c([2H])c(OC)c2c3c1C[C@@H]1N(C)CC[C@]34C([2H])(O2)C([2H])(O)C=C[C@]14C]

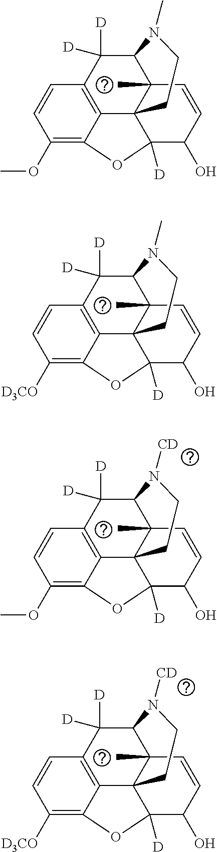

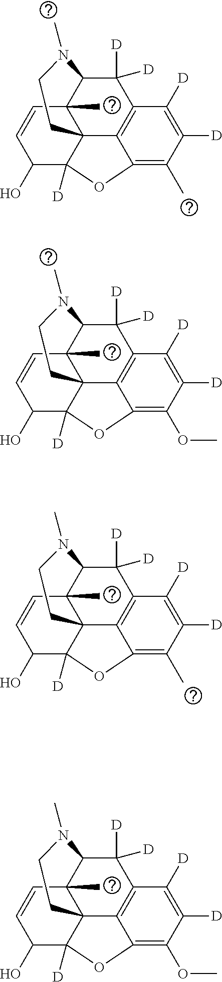 [SMILES: [2H]c1c([2H])c2c3c(c1C)OC1([2H])C(O)C=C[C@]4(C)[C@@H](N(C)CC[C@@]314)C2([2H])[2H] ; [2H]c1c([2H])c2c3c(c1OC)OC1([2H])C(O)C=C[C@]4(C)[C@@H](N(C)CC[C@@]314)C2([2H])[2H] ; [2H]c1c([2H])c2c3c(c1C)OC1([2H])C(O)C=C[C@]4(C)[C@@H](N(C)CC[C@@]314)C2([2H])[2H] ; [2H]c1c([2H])c2c3c(c1OC)OC1([2H])C(O)C=C[C@]4(C)[C@@H](N(C)CC[C@@]314)C2([2H])[2H]]

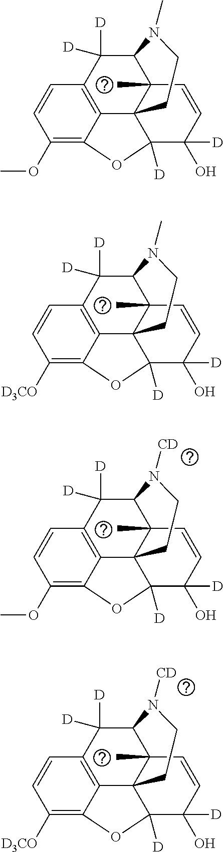

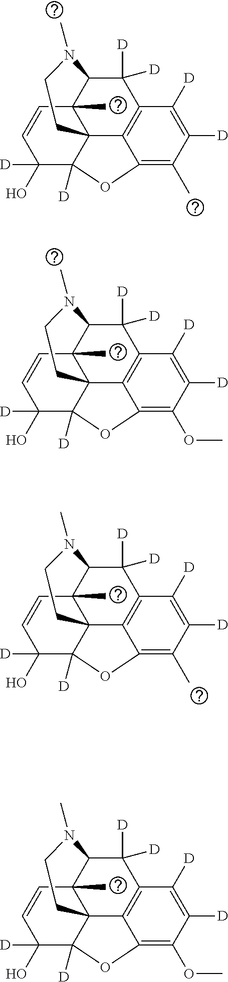 [SMILES: [2H]c1c([2H])c2c3c(c1C)OC1([2H])C([2H])(O)C=C[C@]4(C)[C@@H](N(C)CC[C@@]314)C2([2H])[2H] ; [2H]c1c([2H])c2c3c(c1OC)OC1([2H])C([2H])(O)C=C[C@]4(C)[C@@H](N(C)CC[C@@]314)C2([2H])[2H] ; [2H]c1c([2H])c2c3c(c1C)OC1([2H])C([2H])(O)C=C[C@]4(C)[C@@H](N(C)CC[C@@]314)C2([2H])[2H] ; [2H]c1c([2H])c2c3c(c1OC)OC1([2H])C([2H])(O)C=C[C@]4(C)[C@@H](N(C)CC[C@@]314)C2([2H])[2H]]

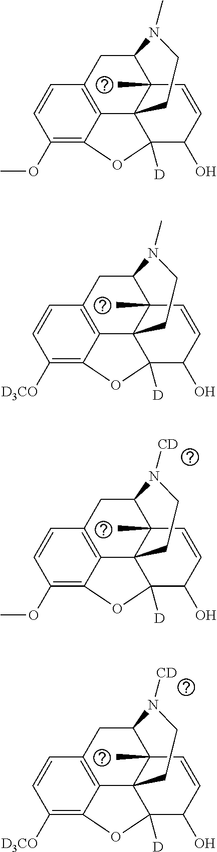

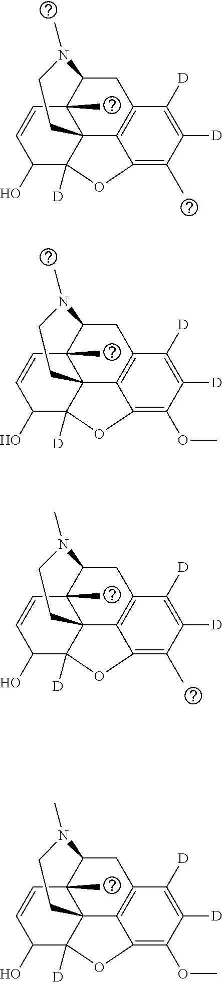 [SMILES: [2H]c1c([2H])c2c3c(c1C)OC1([2H])C(O)C=C[C@]4(C)[C@H](C2)N(C)CC[C@@]314 ; [2H]c1c([2H])c(OC)c2c3c1C[C@@H]1N(C)CC[C@]34C([2H])(O2)C(O)C=C[C@]14C ; [2H]c1c([2H])c2c3c(c1C)OC1([2H])C(O)C=C[C@]4(C)[C@H](C2)N(C)CC[C@@]314 ; [2H]c1c([2H])c(OC)c2c3c1C[C@@H]1N(C)CC[C@]34C([2H])(O2)C(O)C=C[C@]14C]

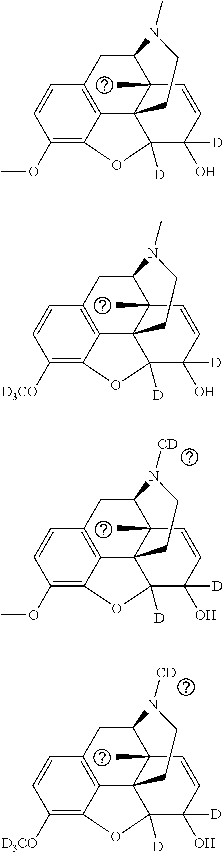

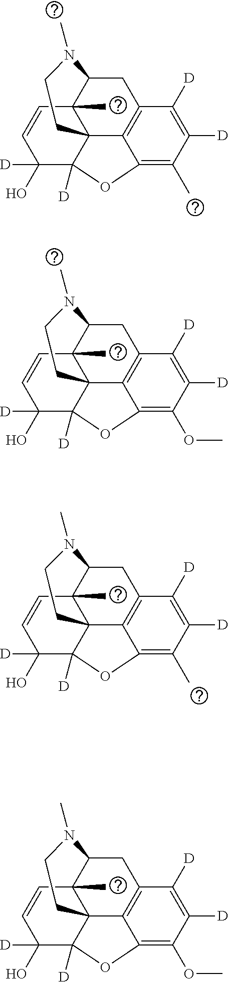 [SMILES: [2H]c1c([2H])c2c3c(c1C)OC1([2H])C([2H])(O)C=C[C@]4(C)[C@H](C2)N(C)CC[C@@]314 ; [2H]c1c([2H])c(OC)c2c3c1C[C@@H]1N(C)CC[C@]34C([2H])(O2)C([2H])(O)C=C[C@]14C ; [2H]c1c([2H])c2c3c(c1C)OC1([2H])C([2H])(O)C=C[C@]4(C)[C@H](C2)N(C)CC[C@@]314 ; [2H]c1c([2H])c(OC)c2c3c1C[C@@H]1N(C)CC[C@]34C([2H])(O2)C([2H])(O)C=C[C@]14C]

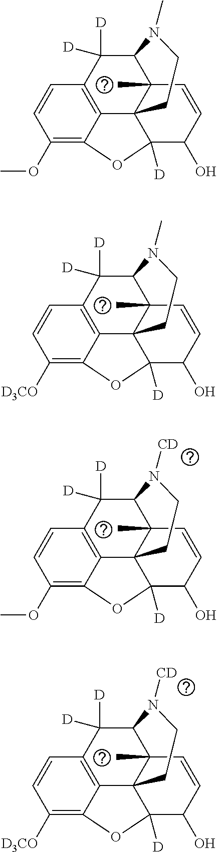

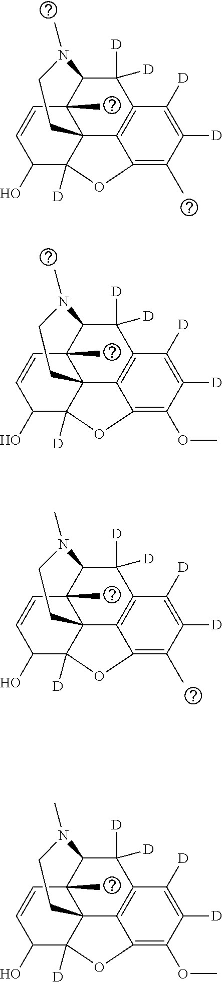 [SMILES: [2H]c1c([2H])c2c3c(c1C)OC1([2H])C(O)C=C[C@]4(C)[C@@H](N(C)CC[C@@]314)C2([2H])[2H] ; [2H]c1c([2H])c2c3c(c1OC)OC1([2H])C(O)C=C[C@]4(C)[C@@H](N(C)CC[C@@]314)C2([2H])[2H] ; [2H]c1c([2H])c2c3c(c1C)OC1([2H])C(O)C=C[C@]4(C)[C@@H](N(C)CC[C@@]314)C2([2H])[2H] ; [2H]c1c([2H])c2c3c(c1OC)OC1([2H])C(O)C=C[C@]4(C)[C@@H](N(C)CC[C@@]314)C2([2H])[2H]]

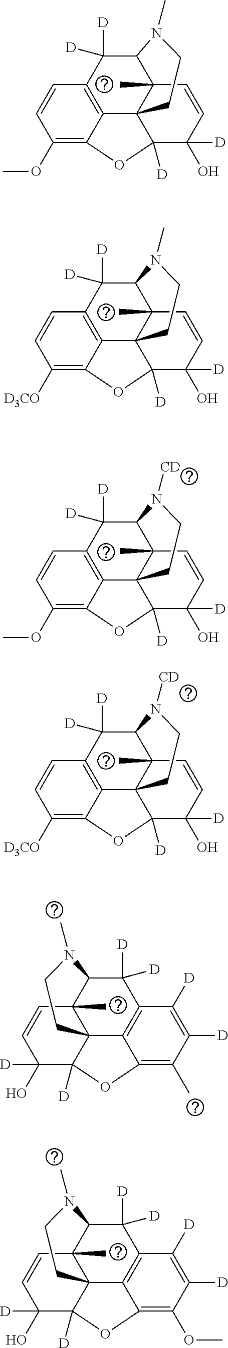

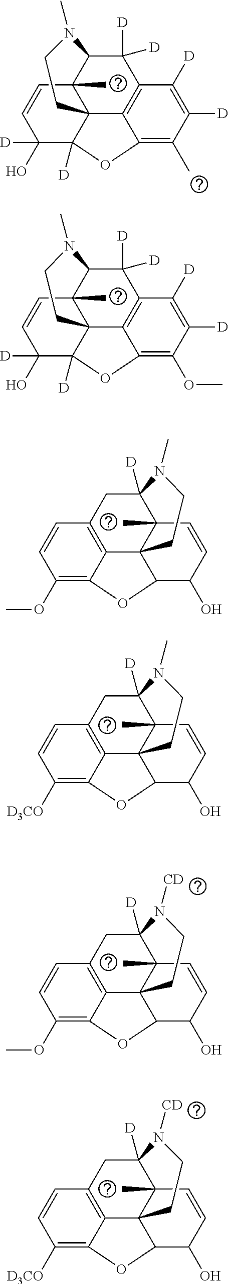

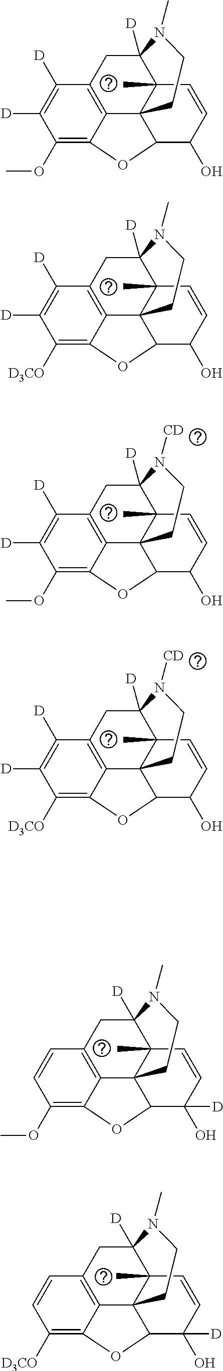

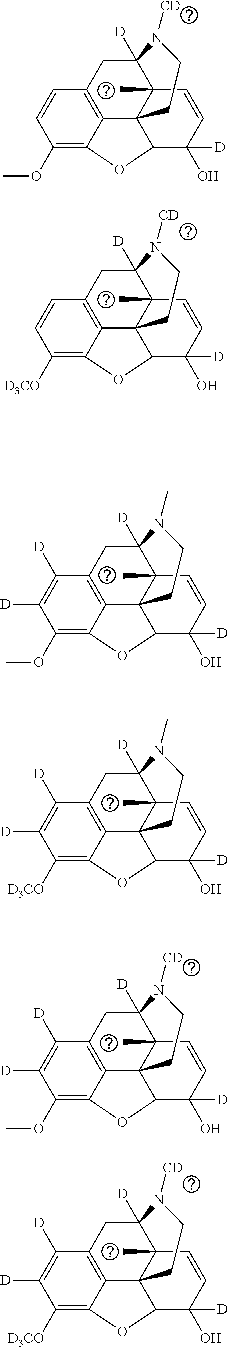

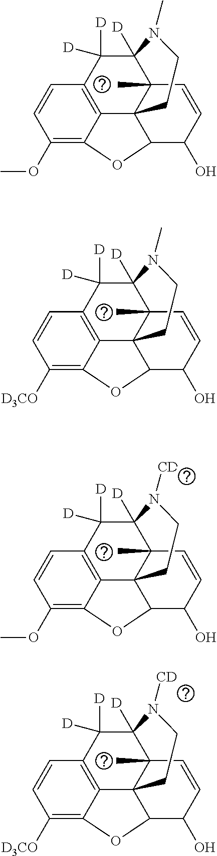

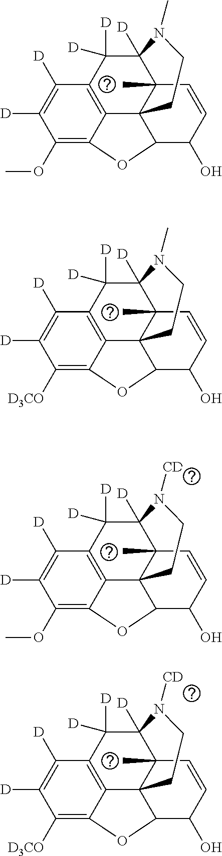

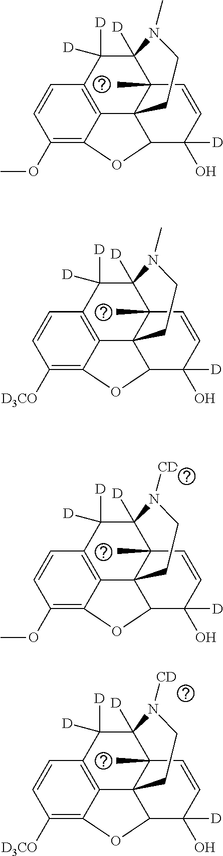

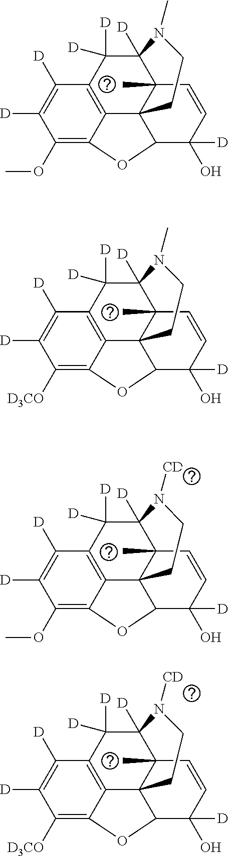

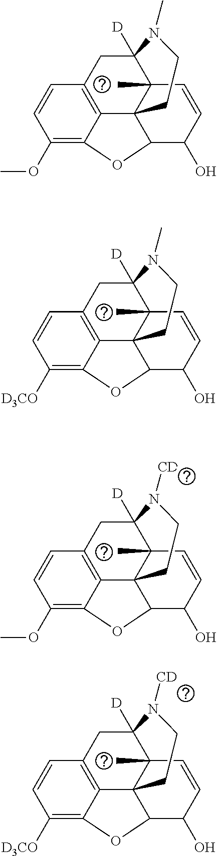

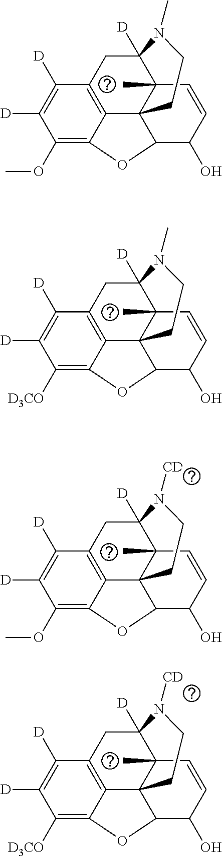

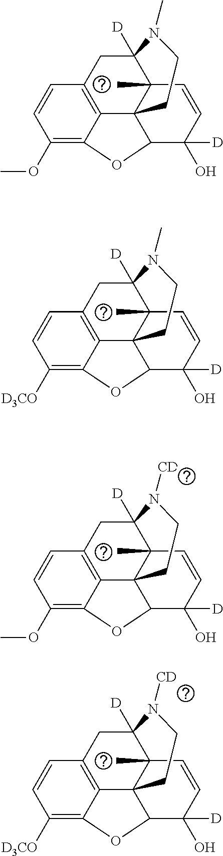

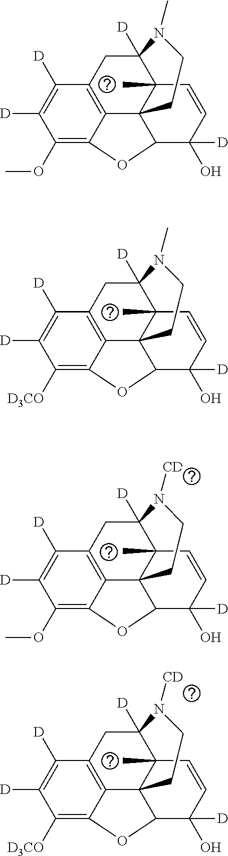

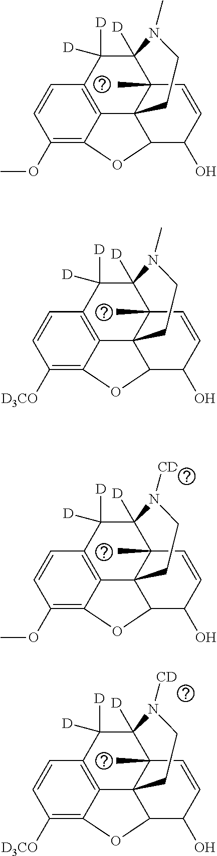

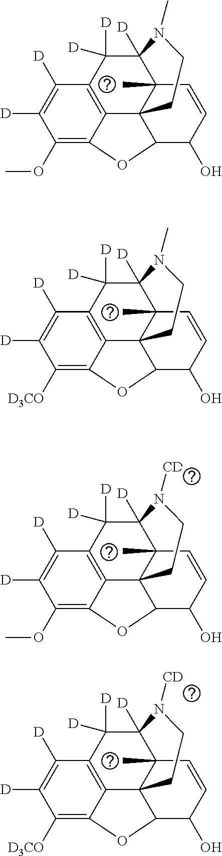

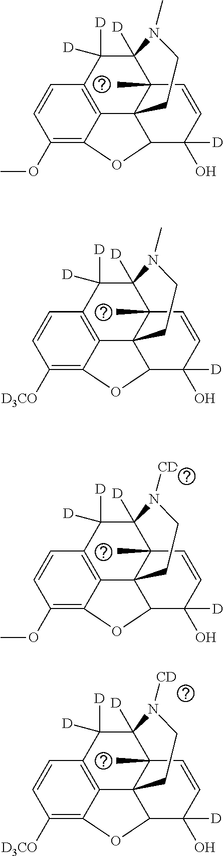

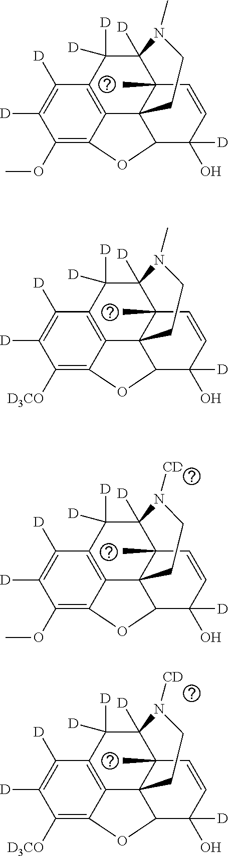

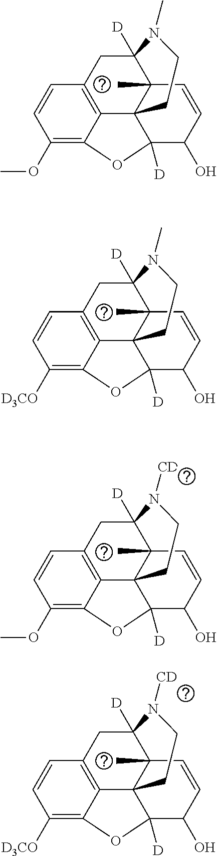

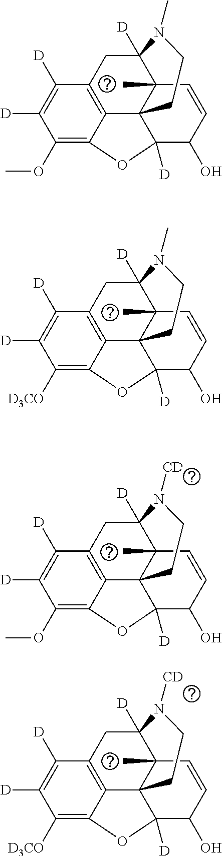

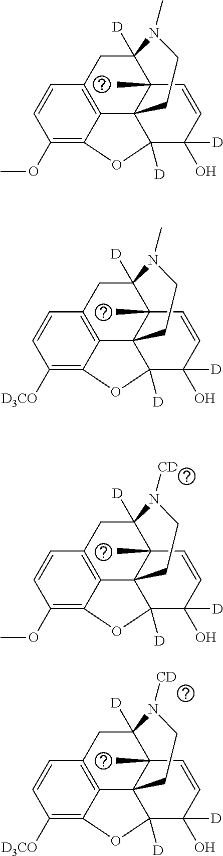

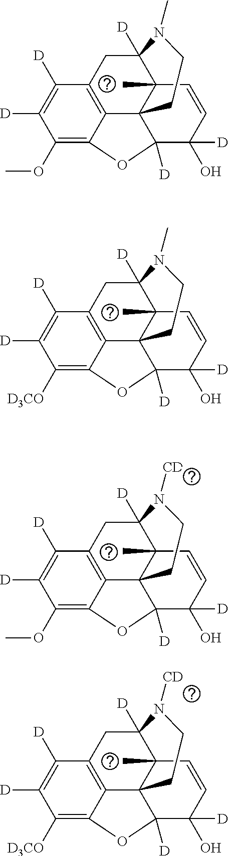

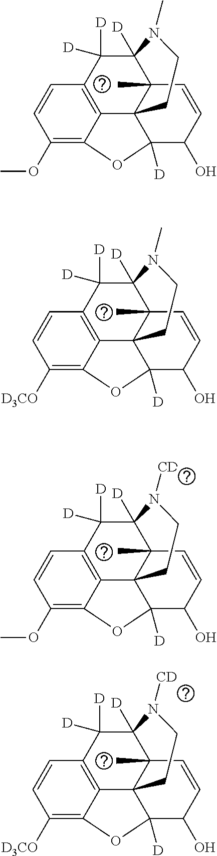

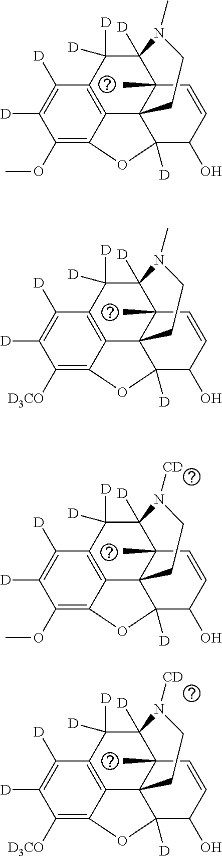

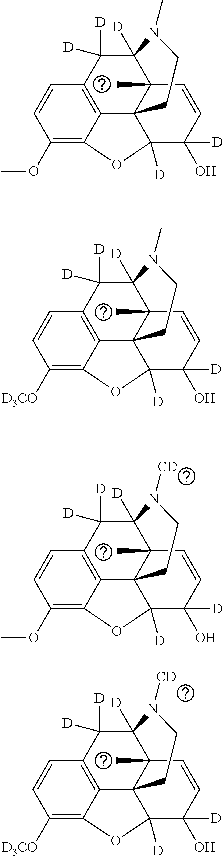

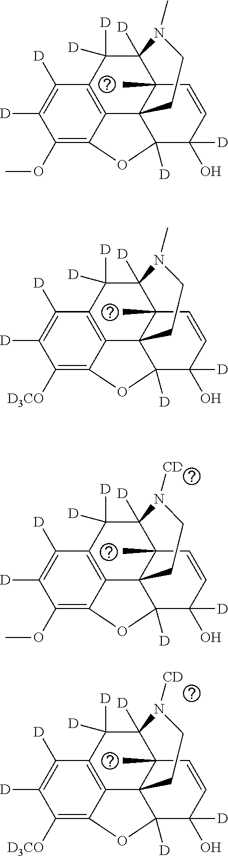

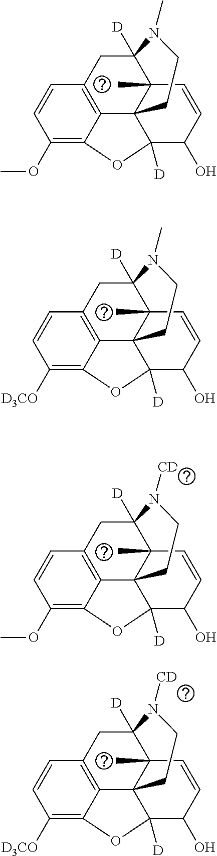

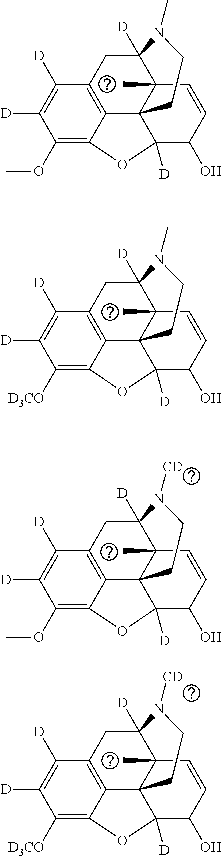

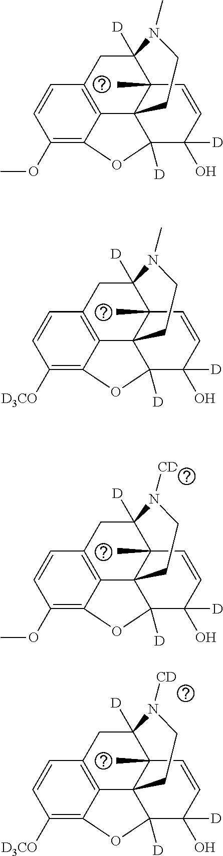

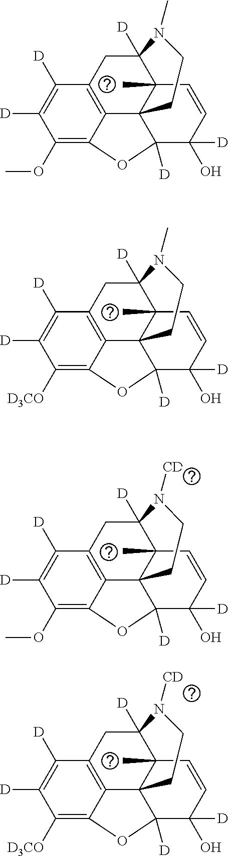

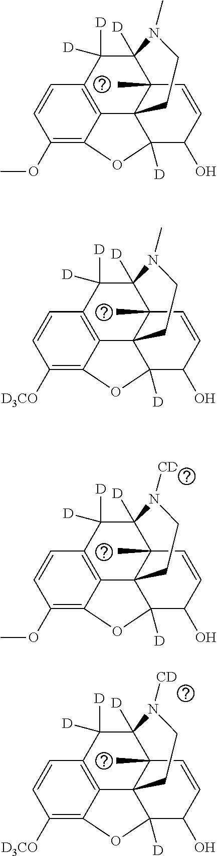

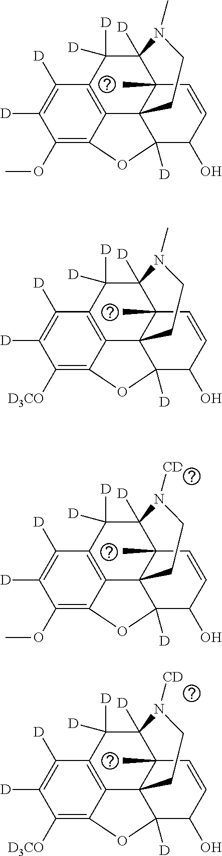

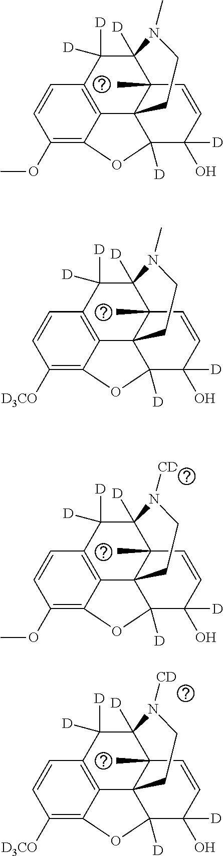

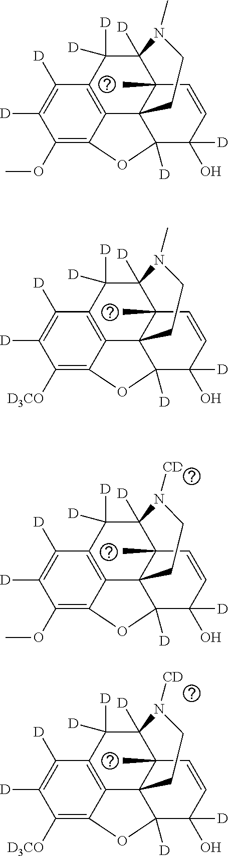

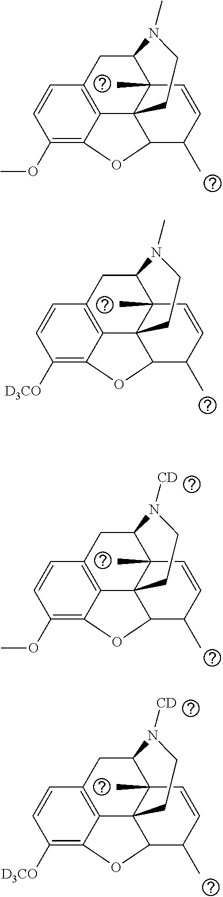

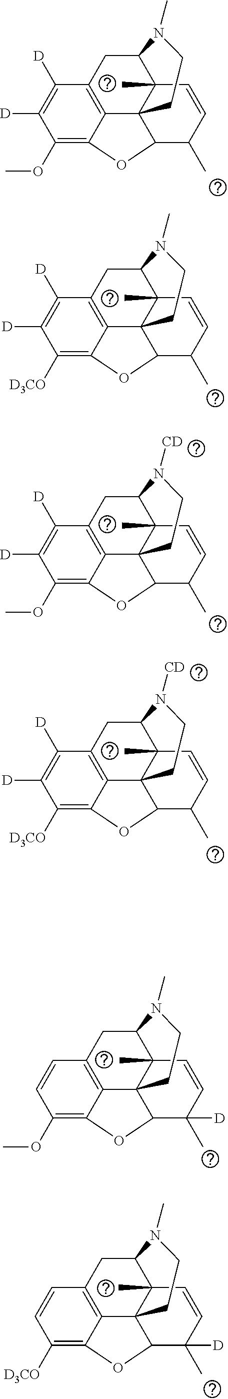

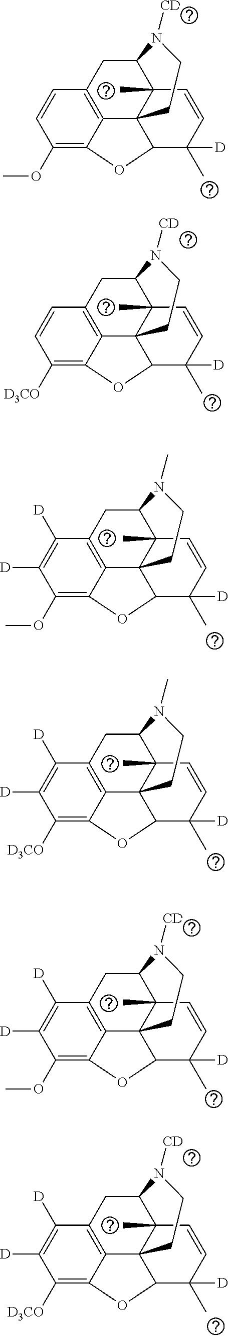

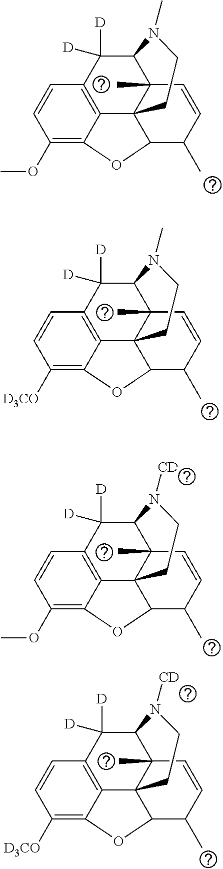

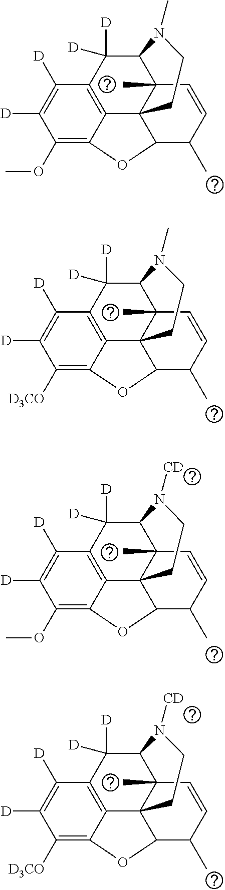

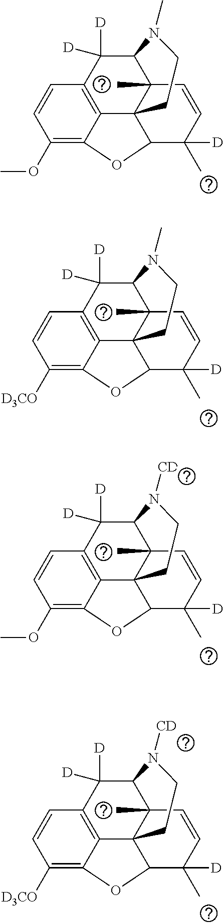

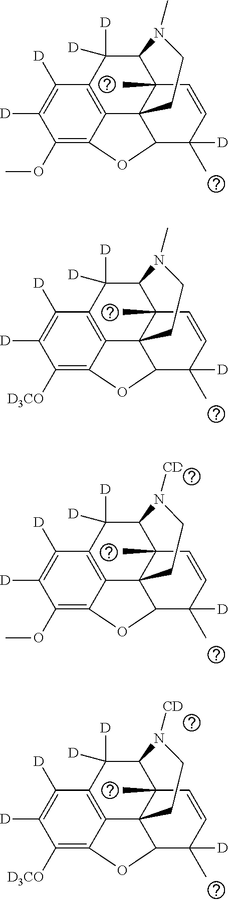

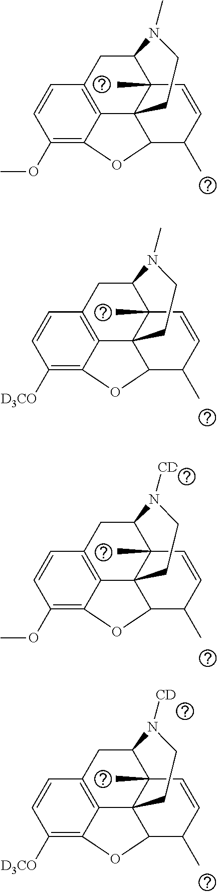

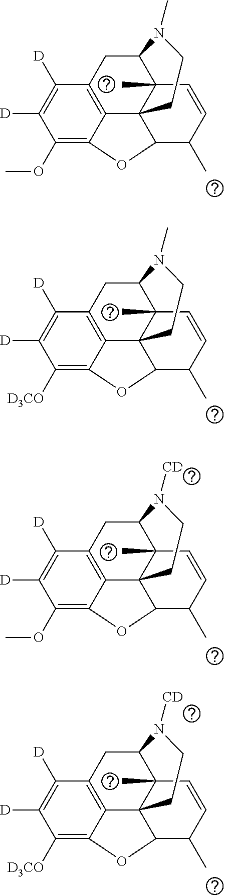

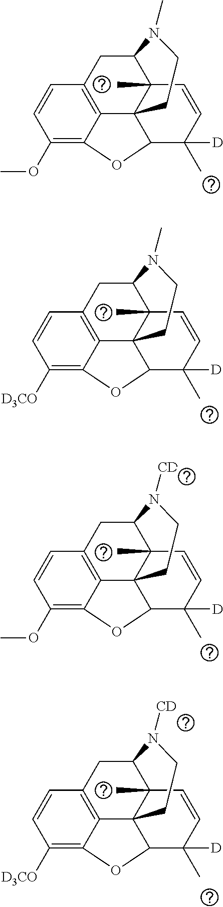

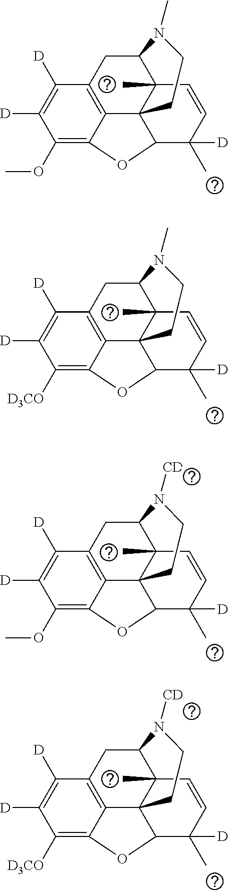

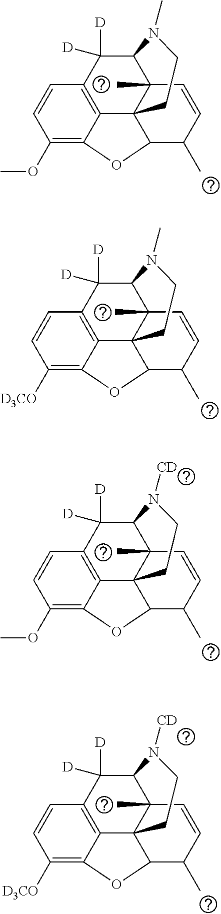

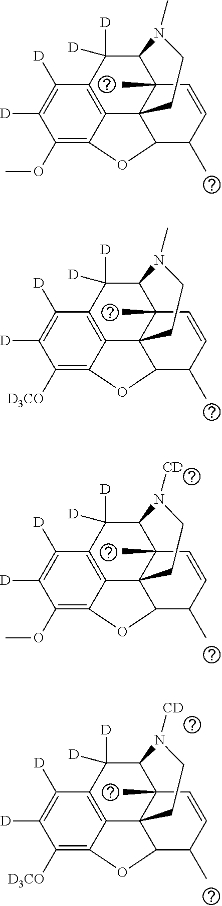

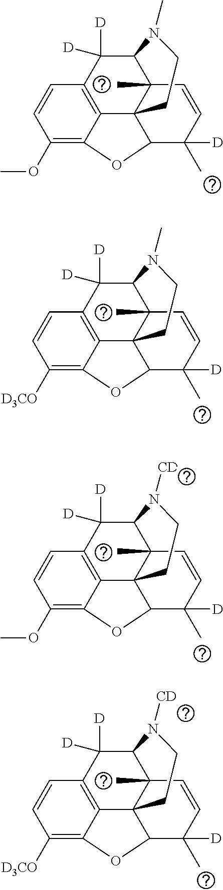

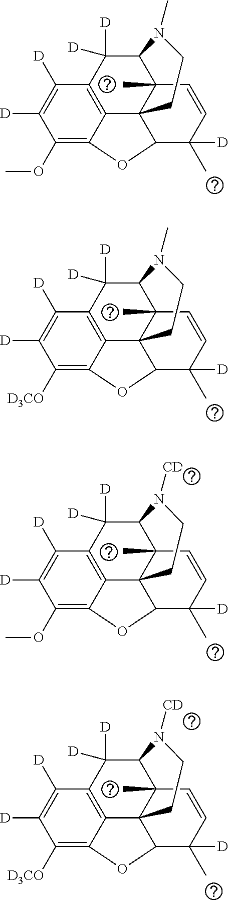

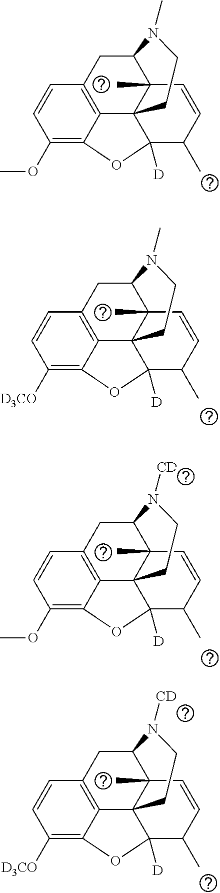

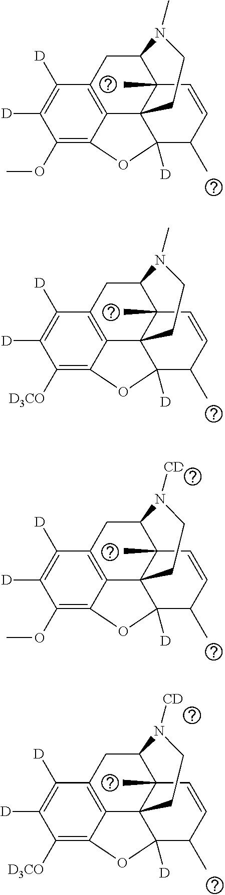

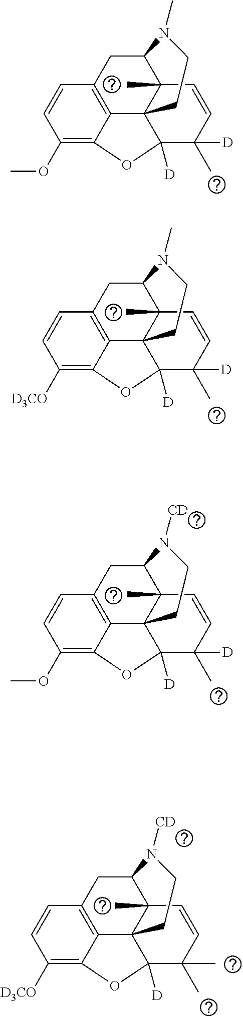

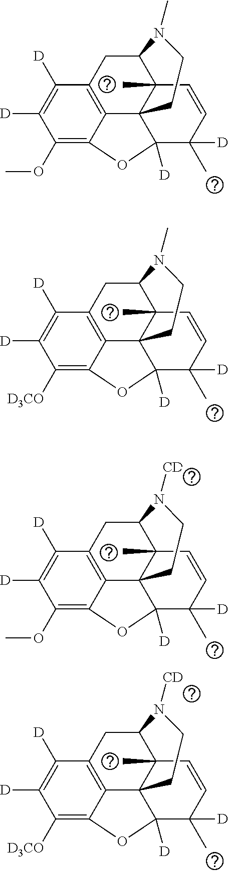

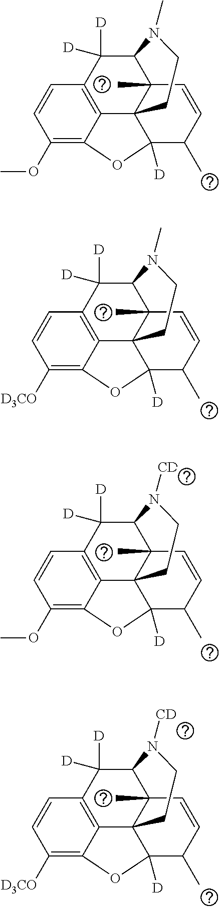

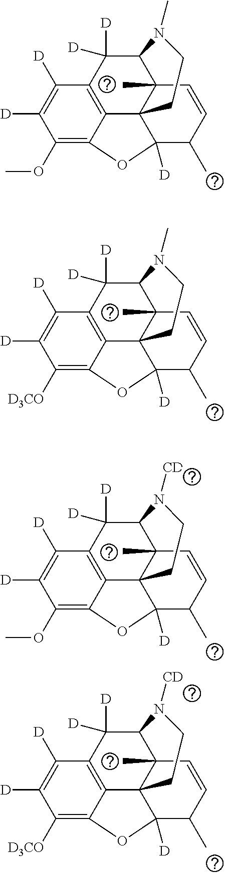

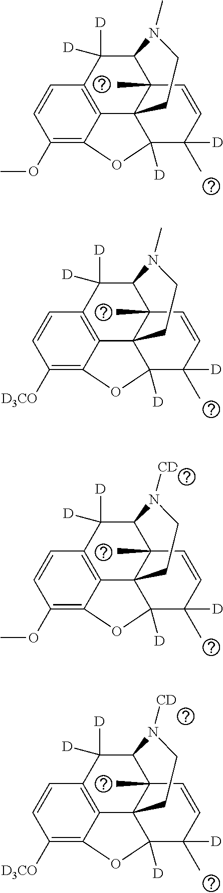

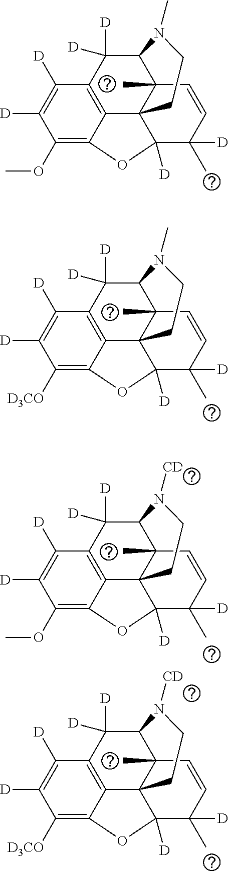

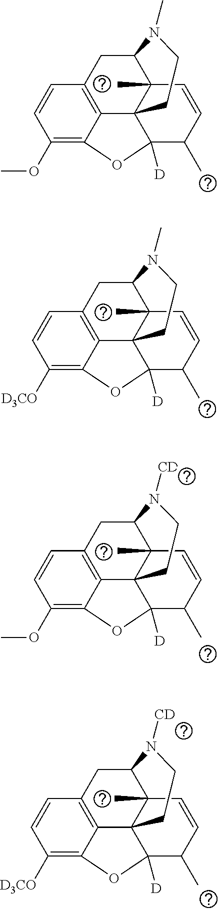

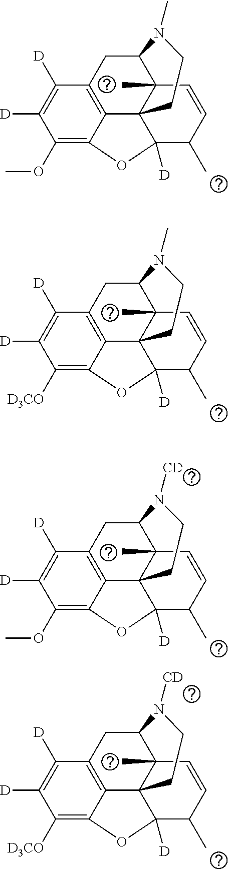

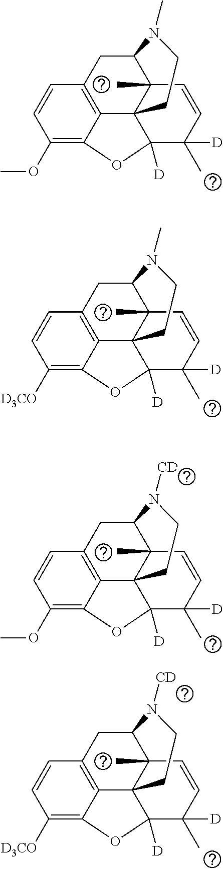

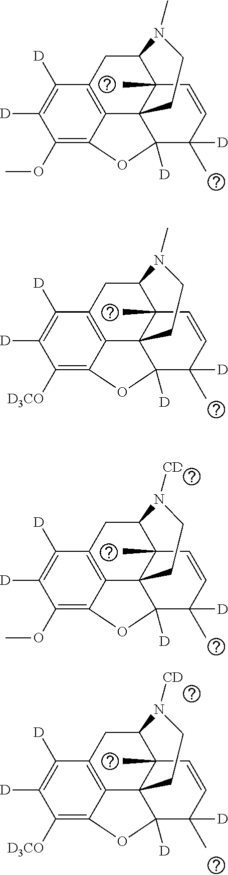

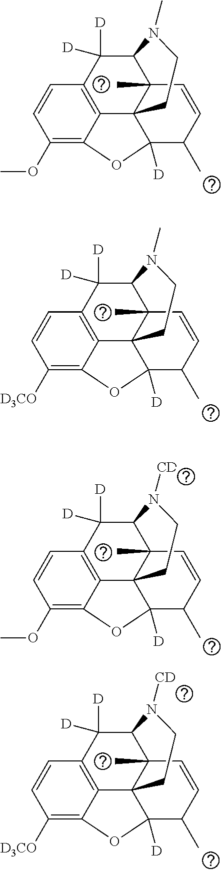

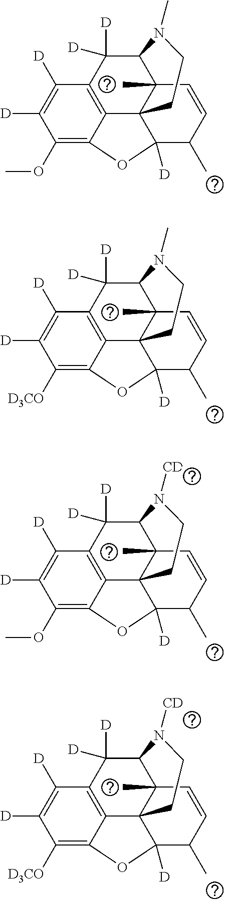

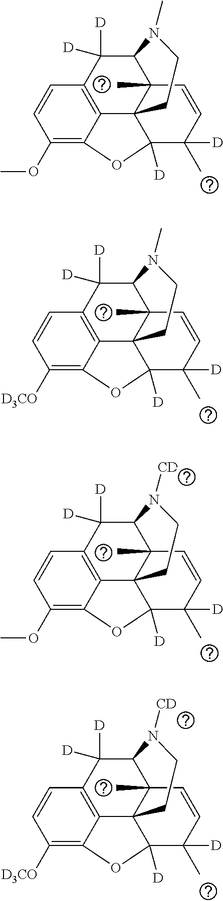

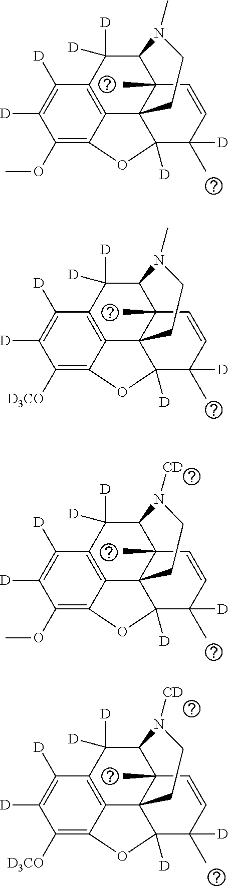

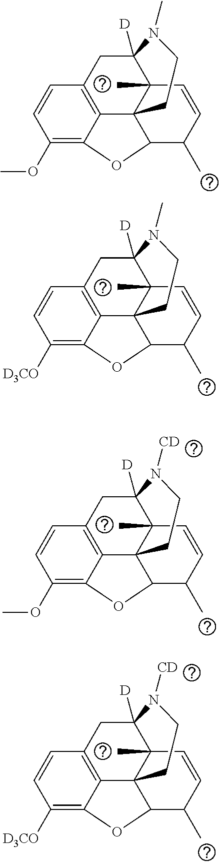

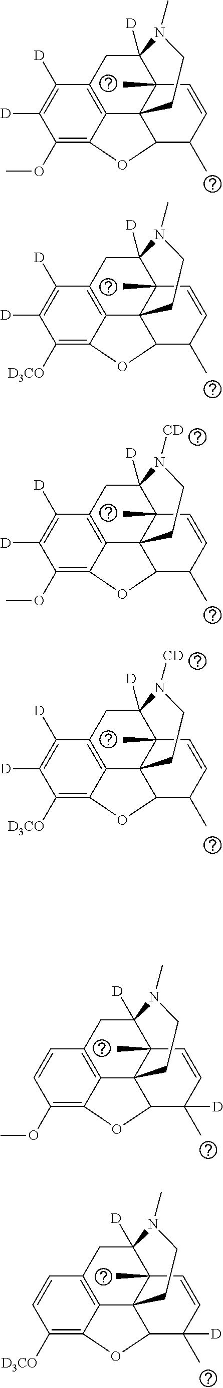

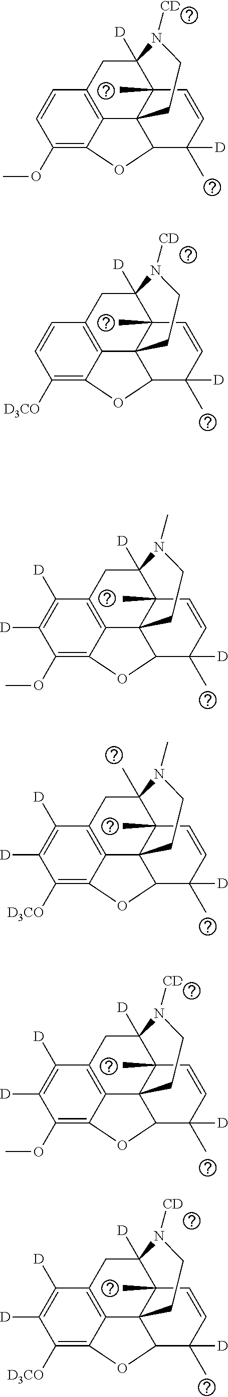

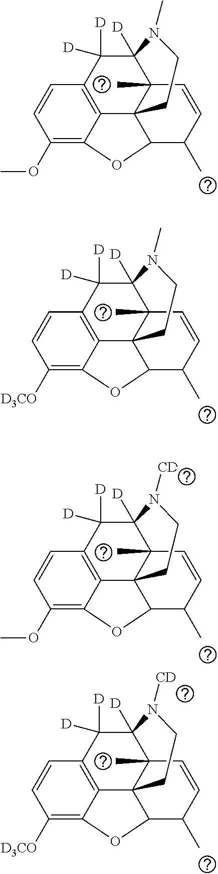

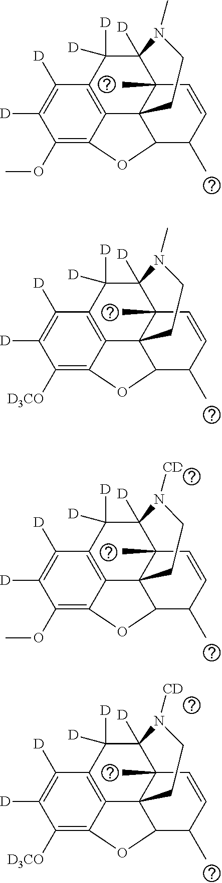

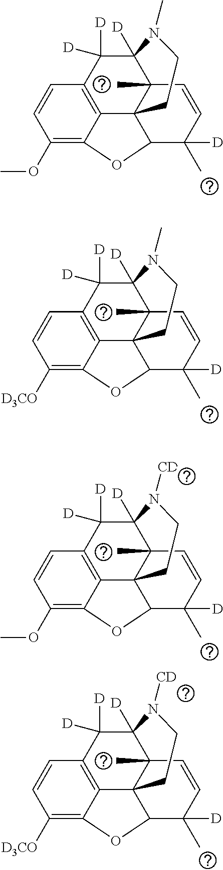

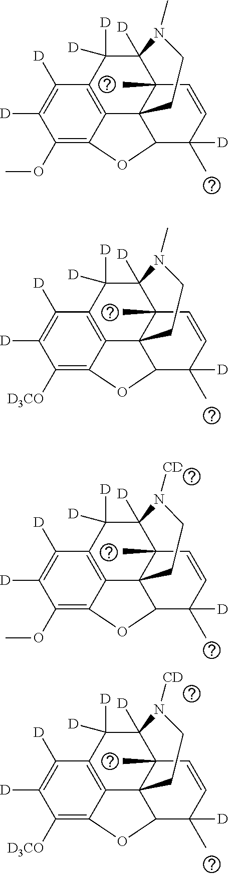

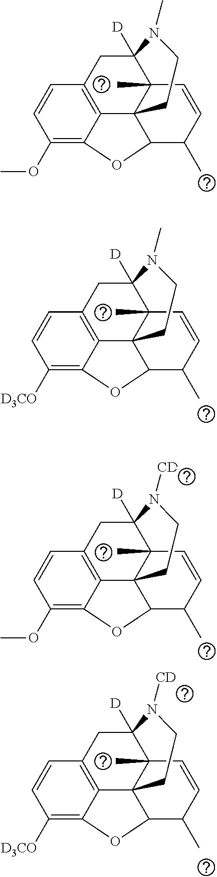

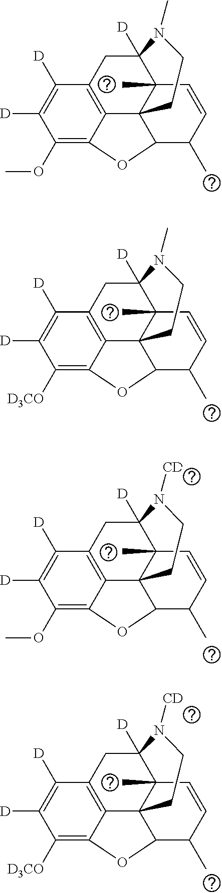

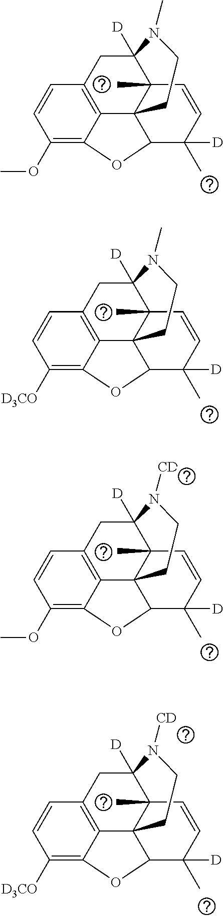

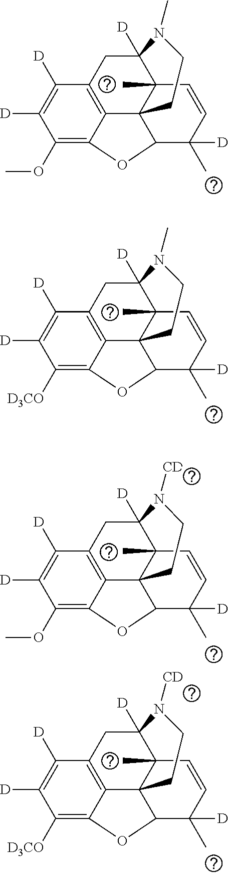

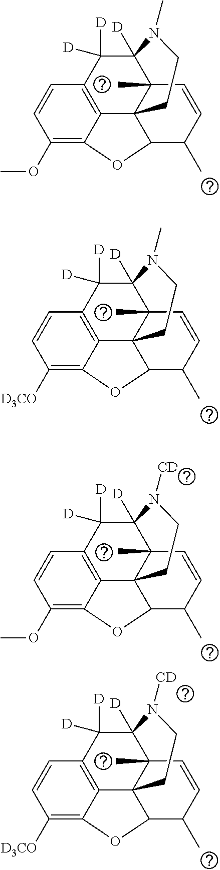

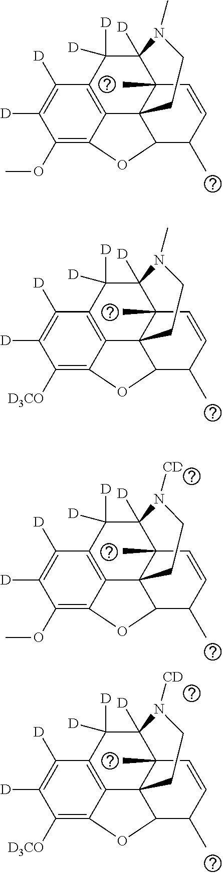

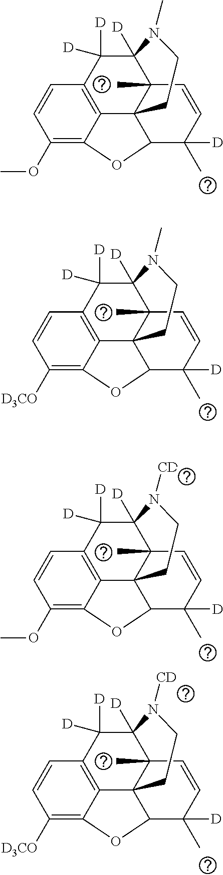

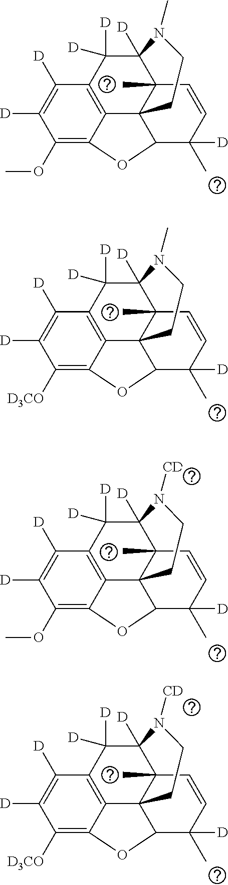

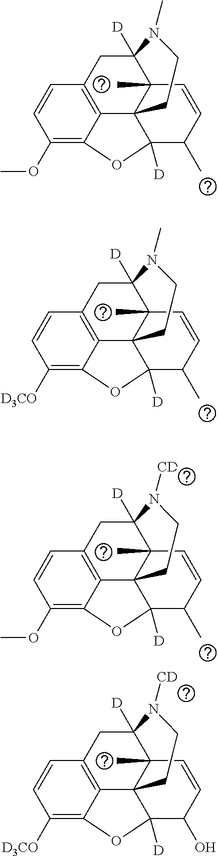

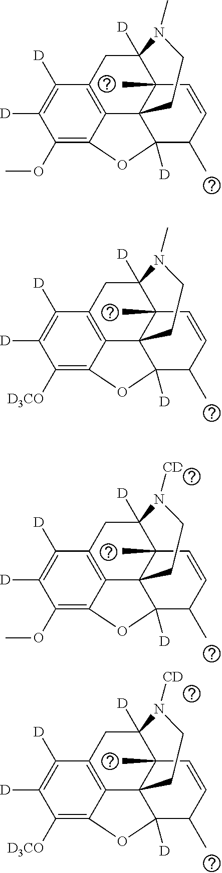

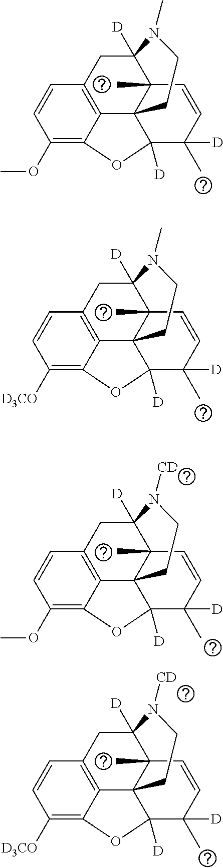

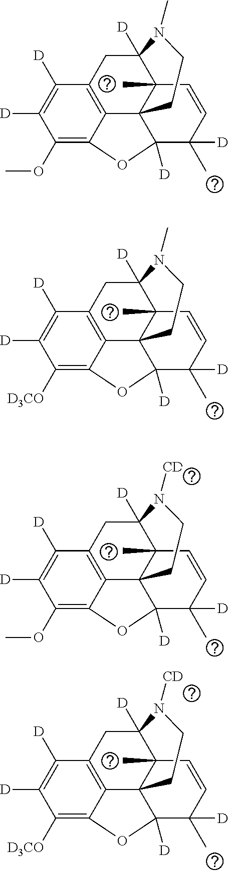

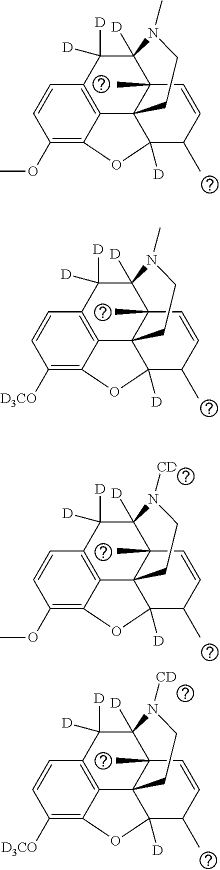

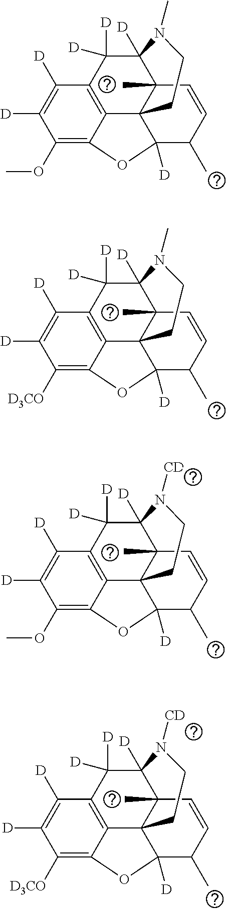

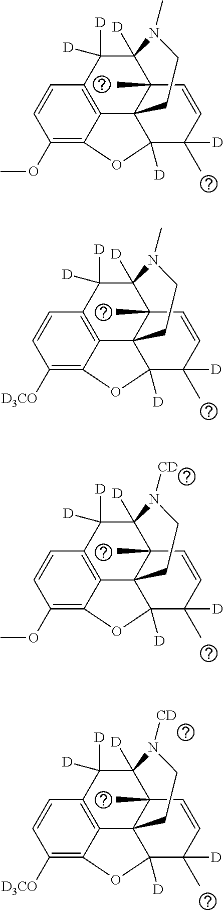

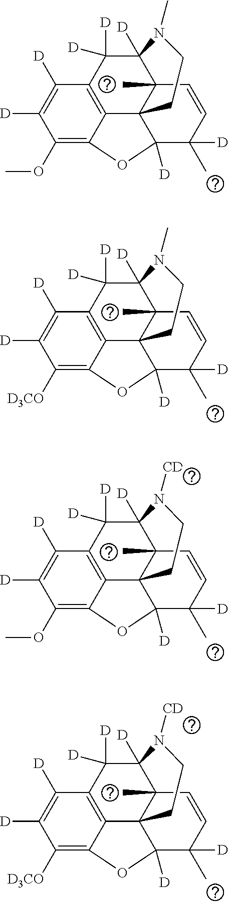

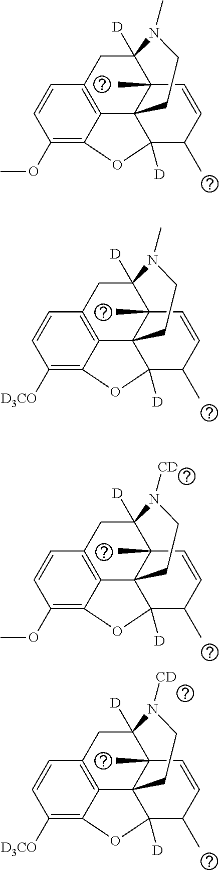

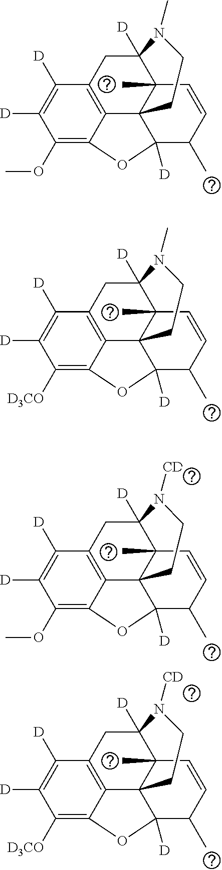

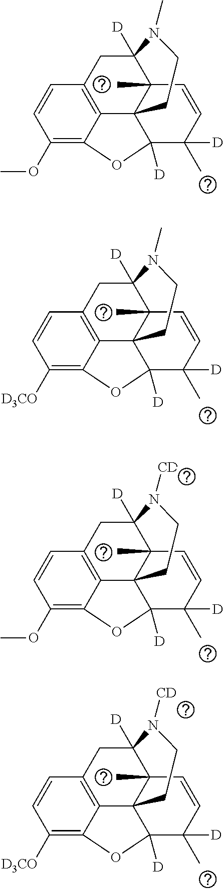

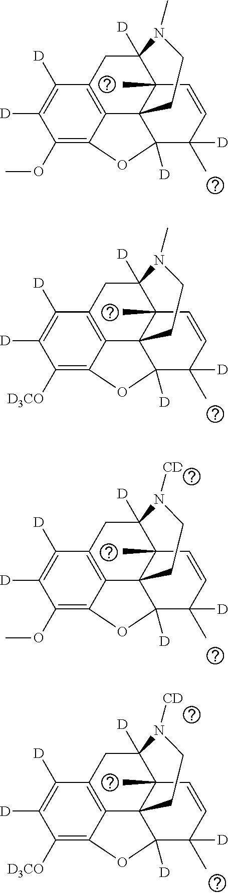

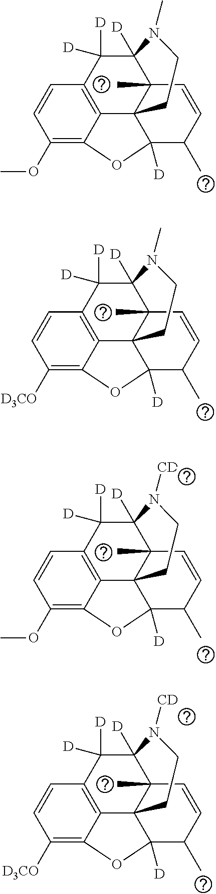

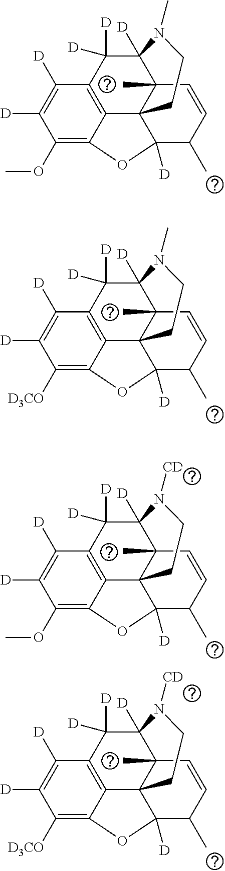

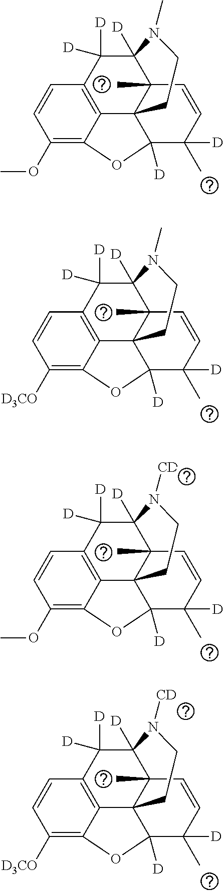

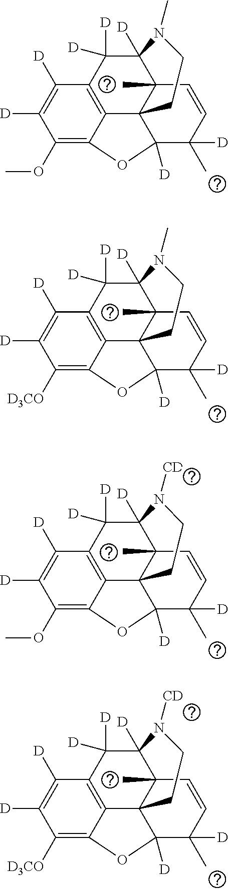

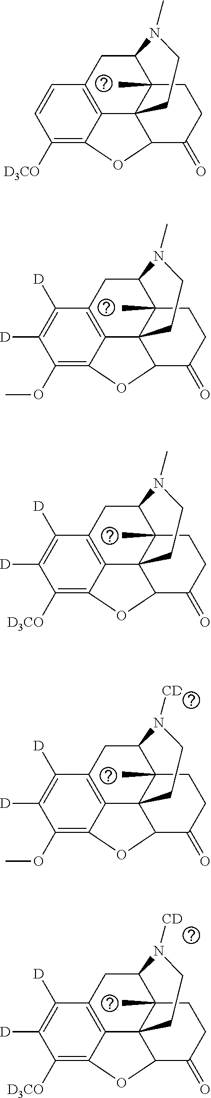

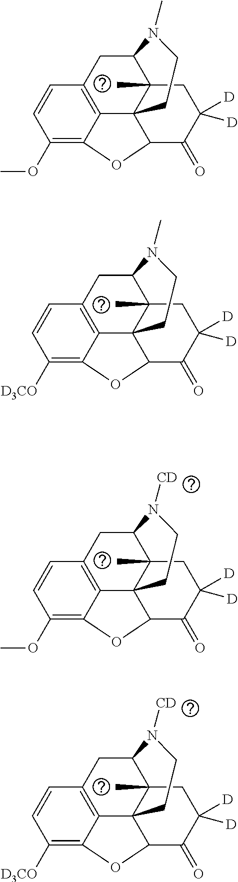

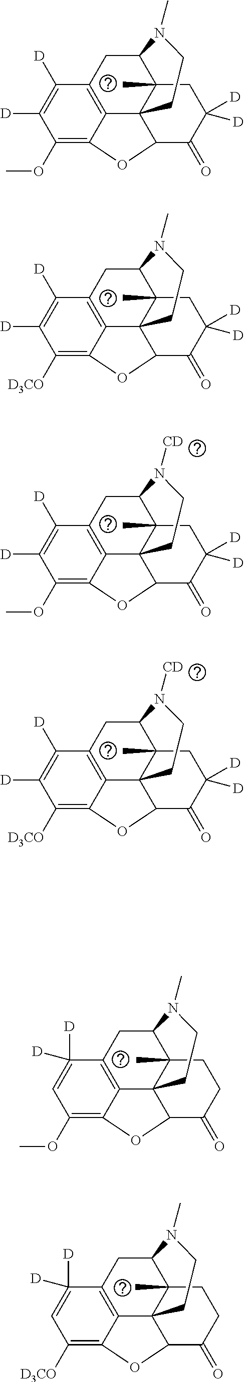

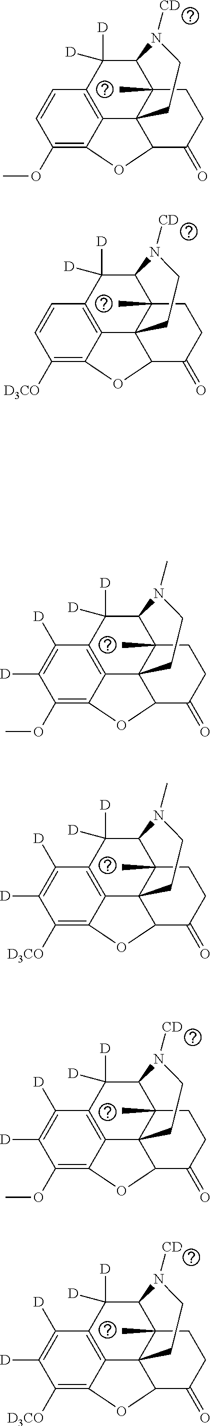

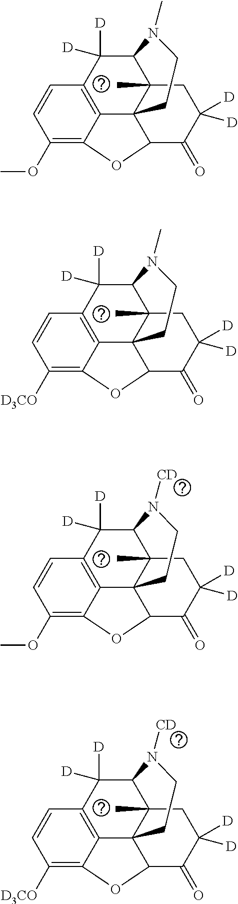

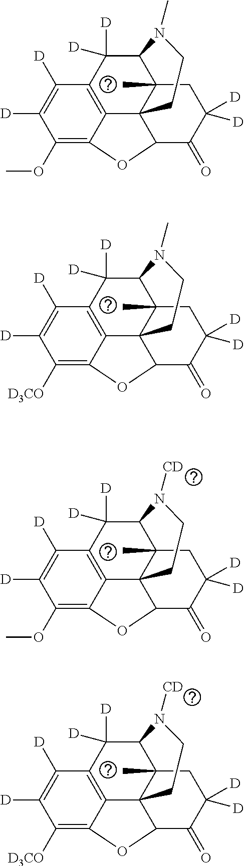

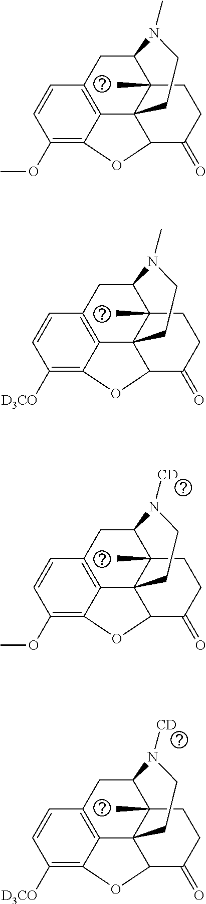

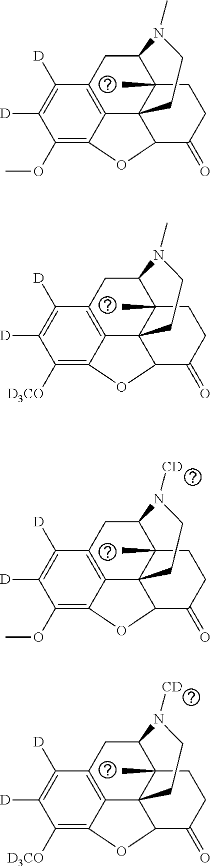

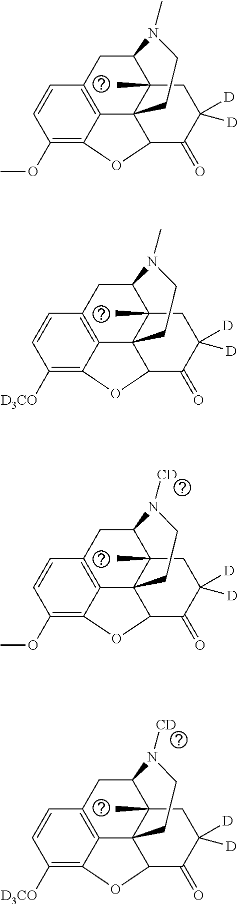

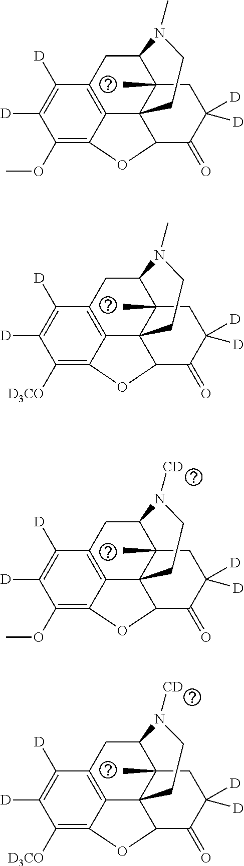

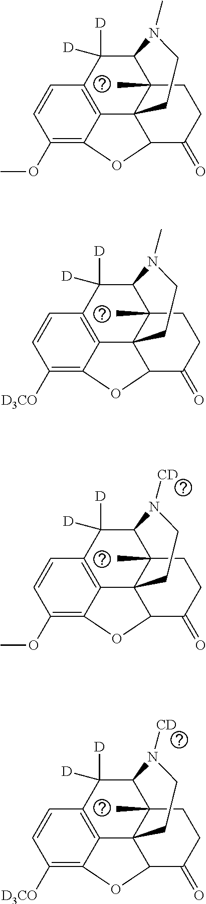

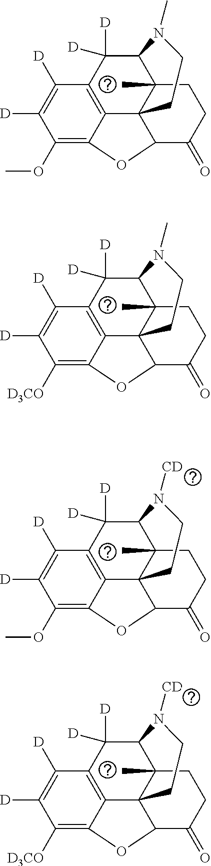

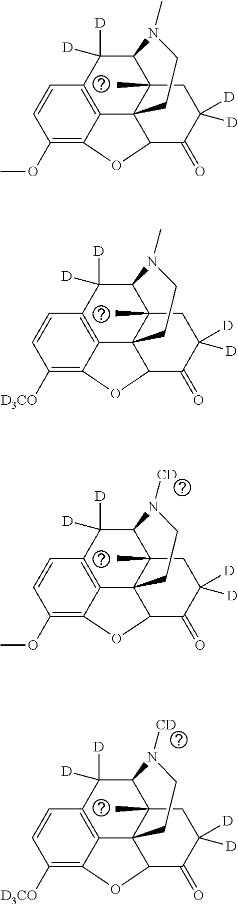

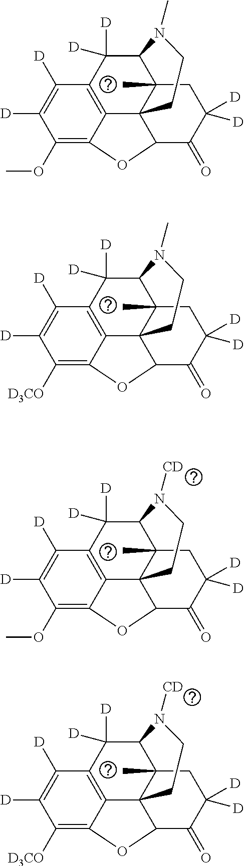

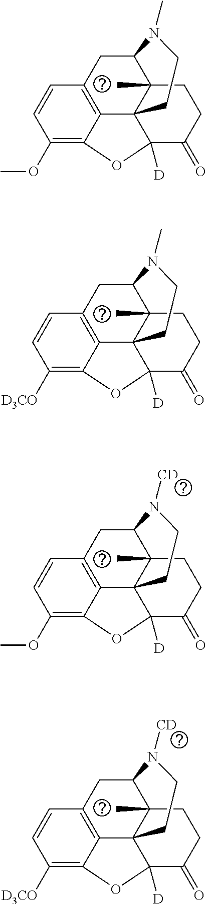

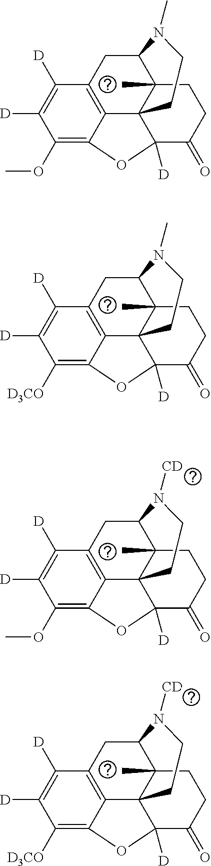

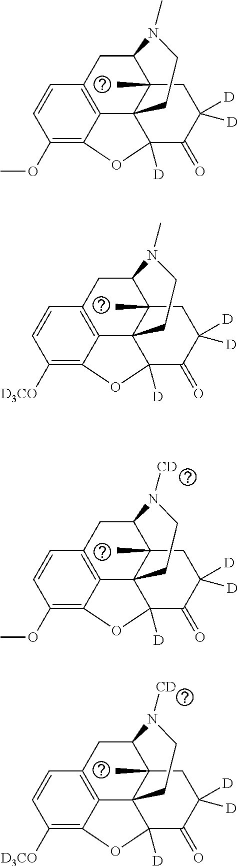

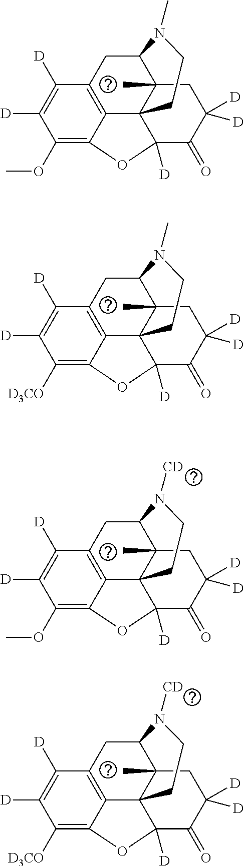

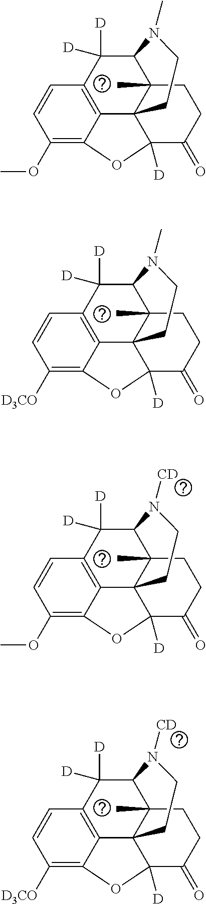

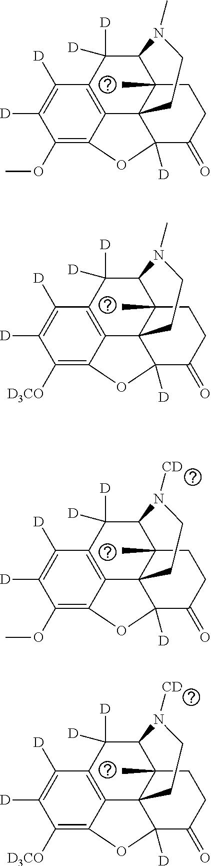

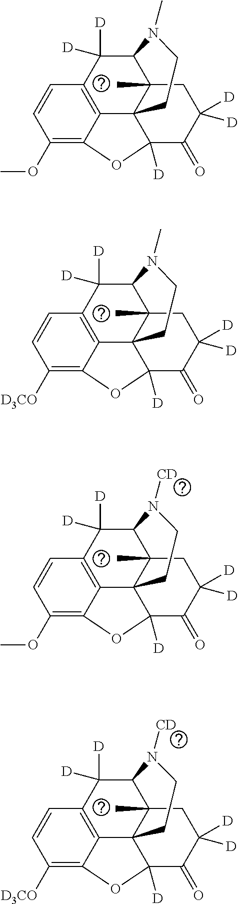

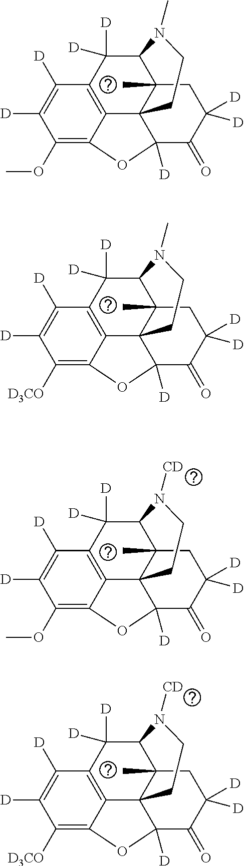

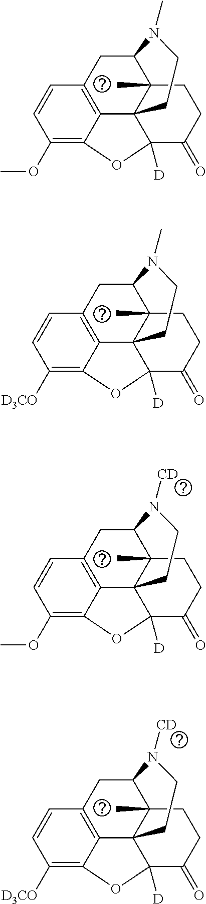

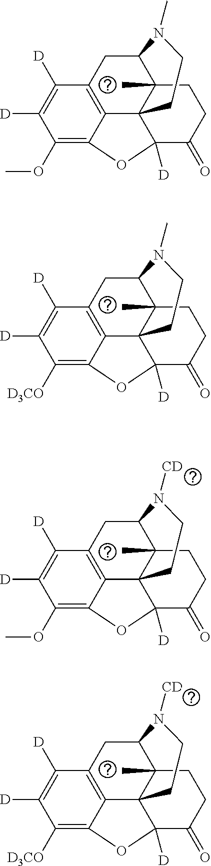

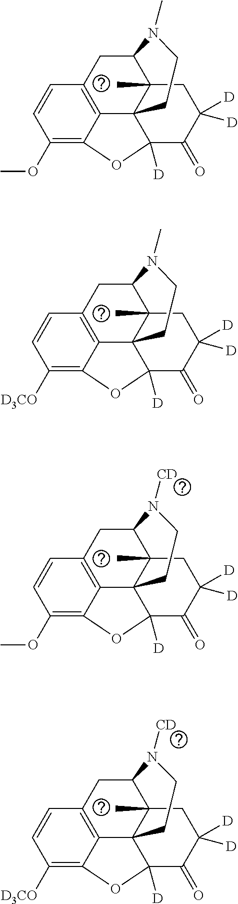

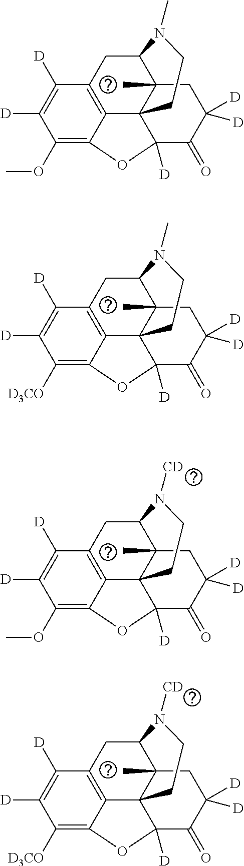

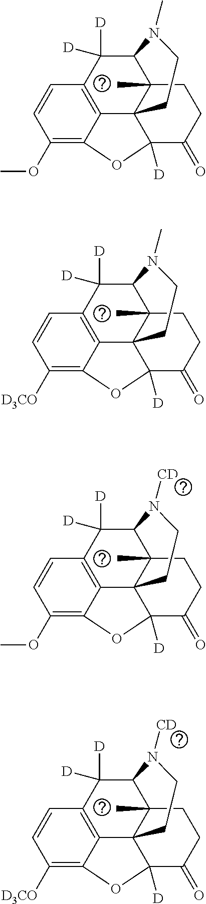

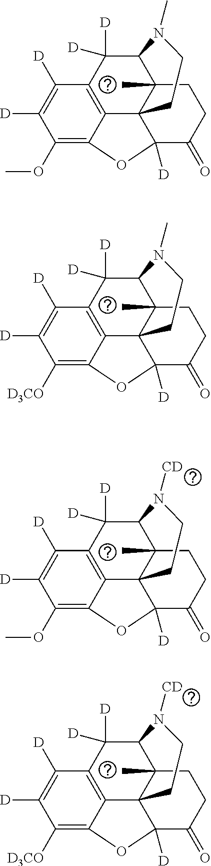

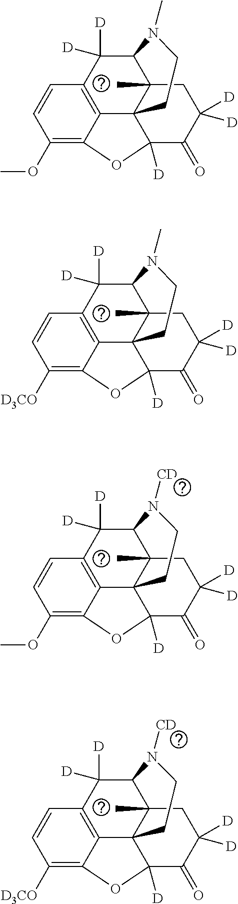

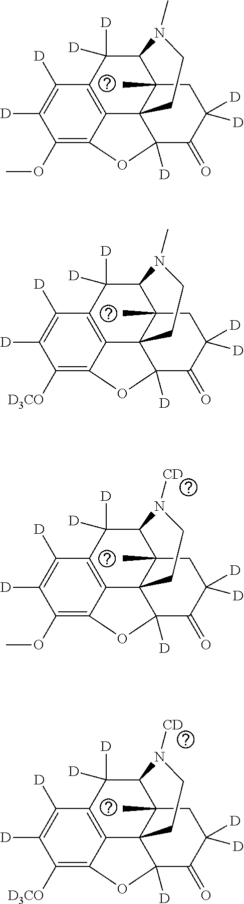

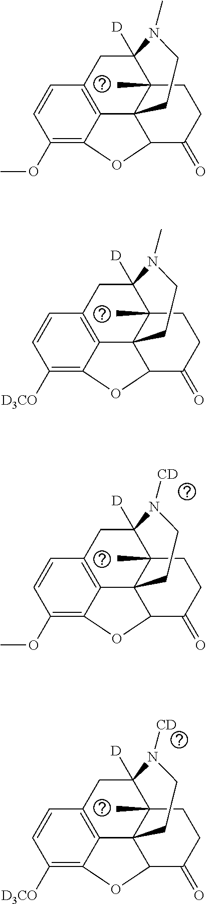

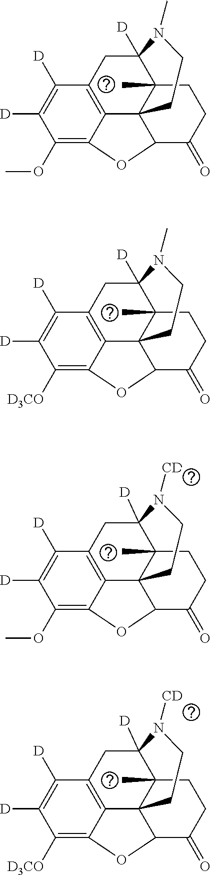

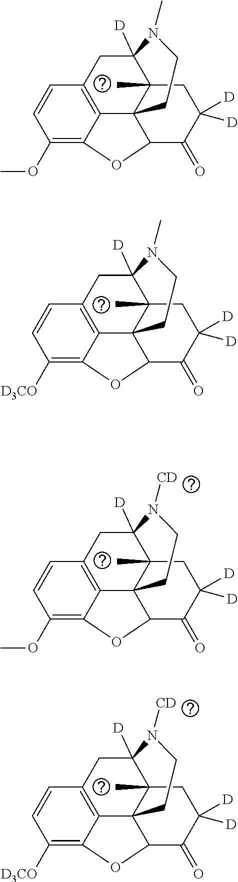

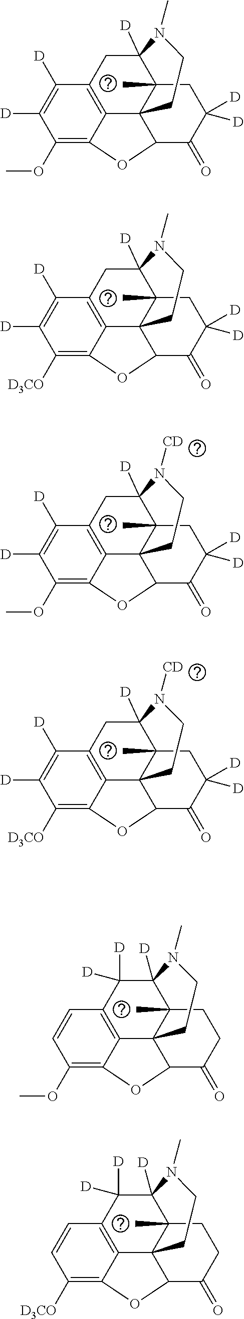

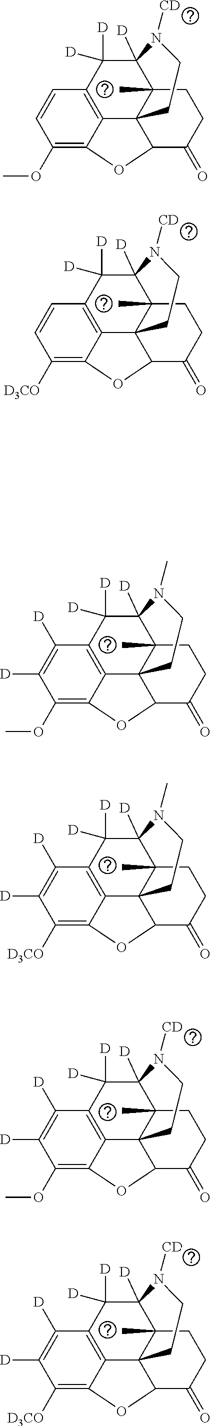

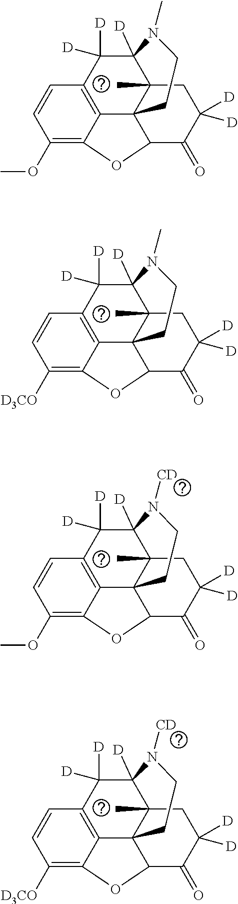

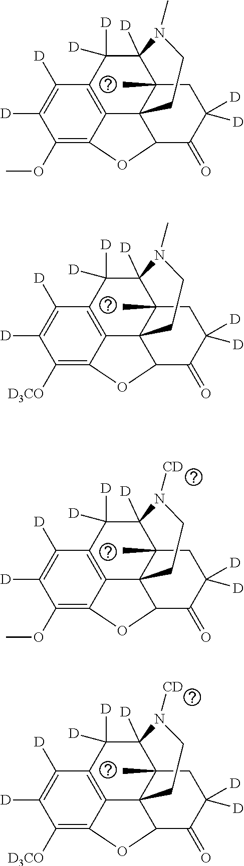

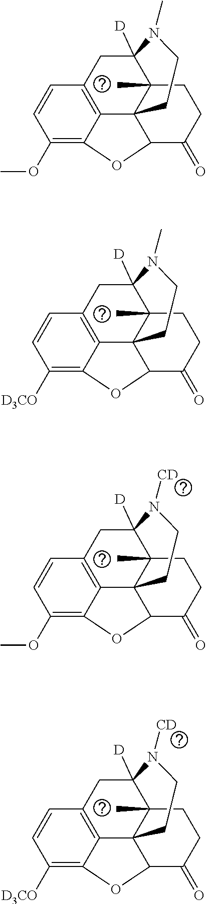

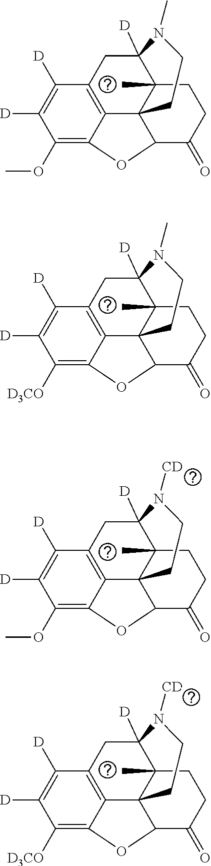

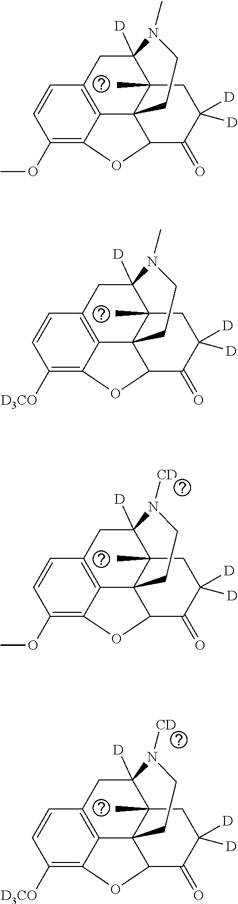

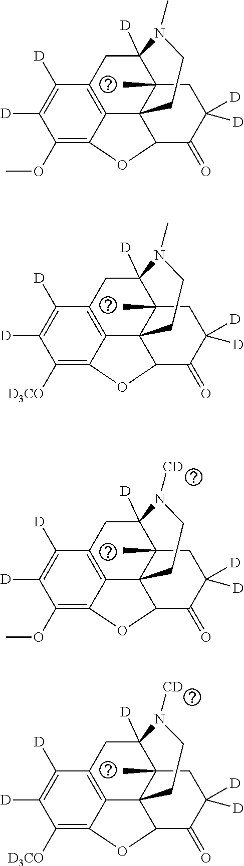

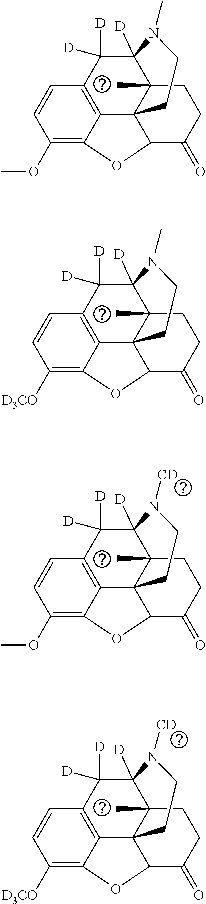

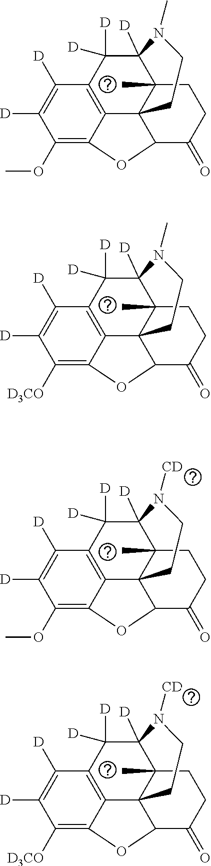

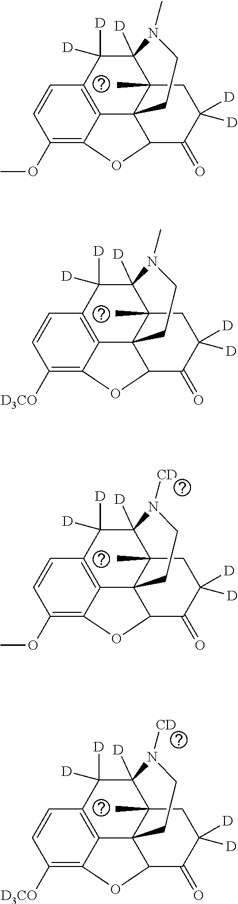

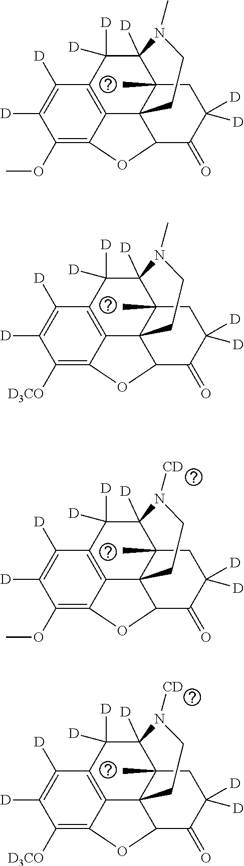

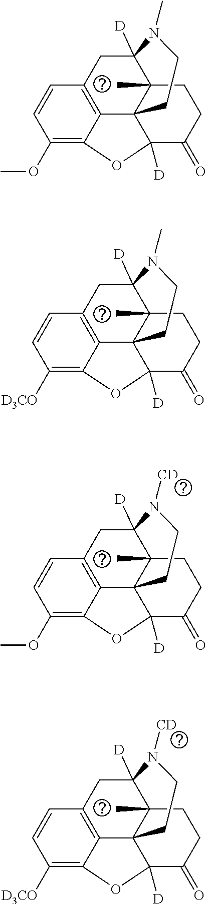

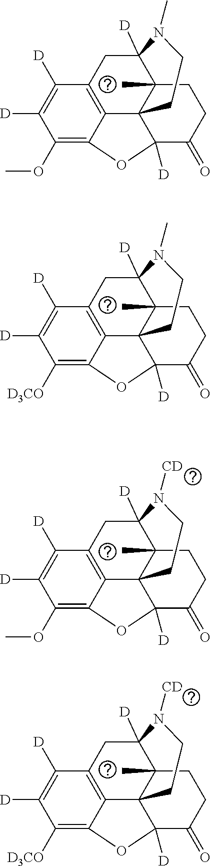

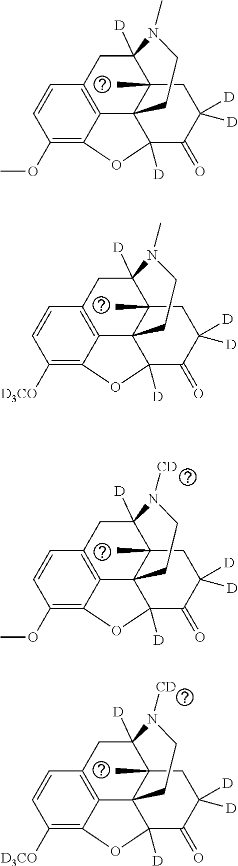

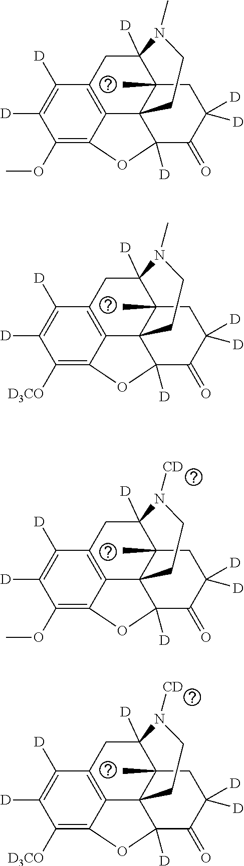

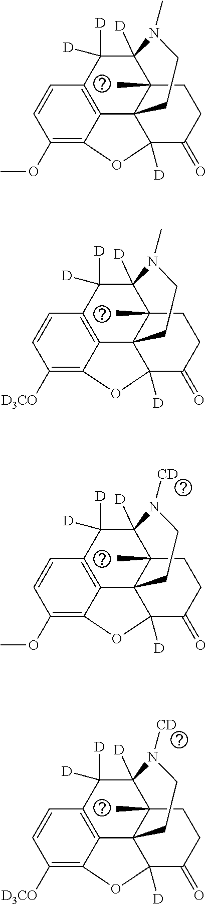

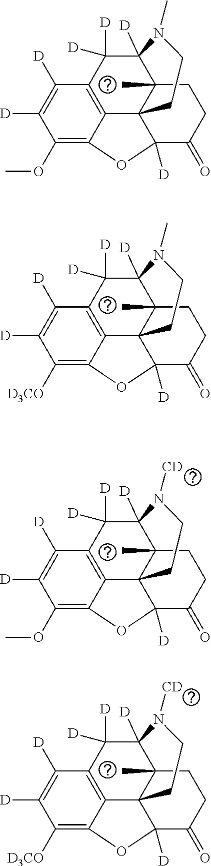

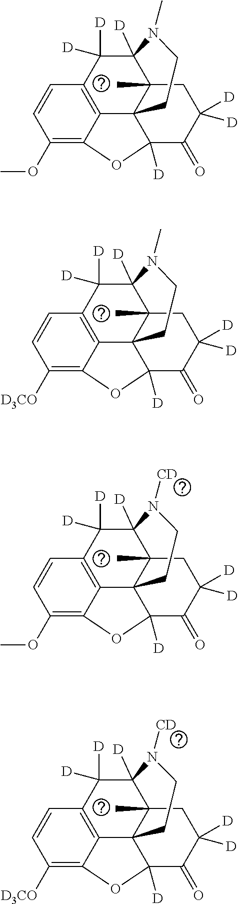

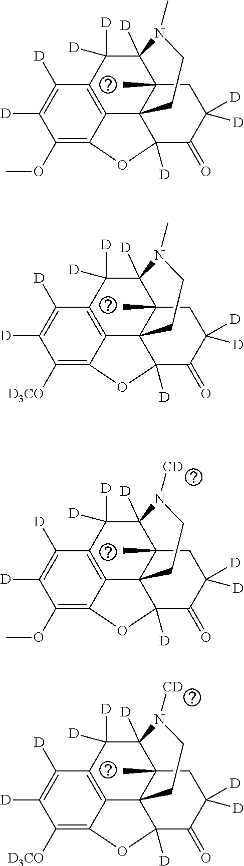

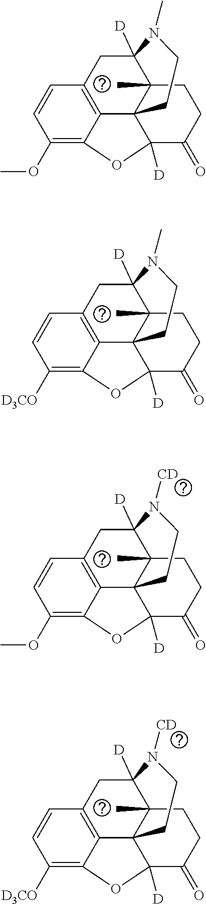

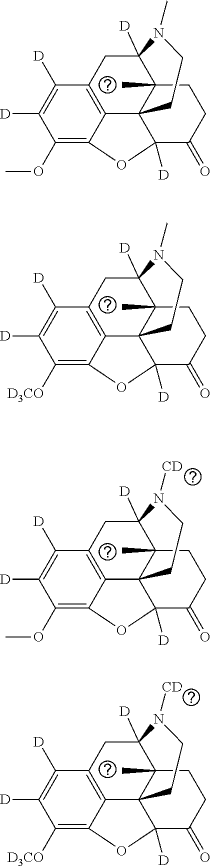

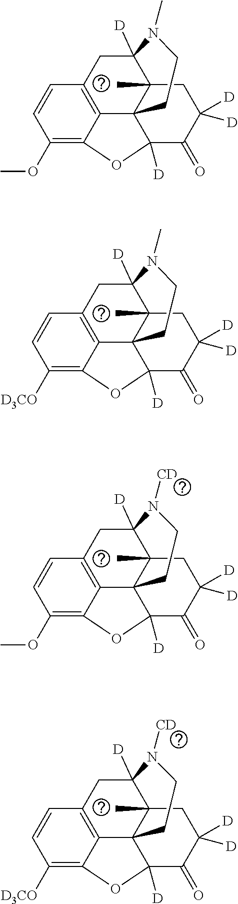

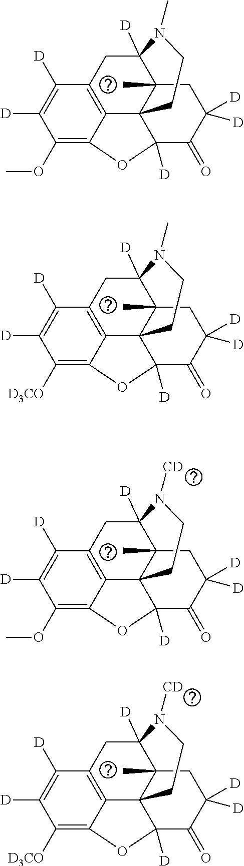

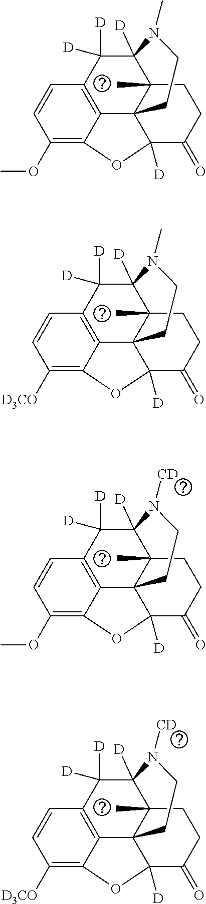

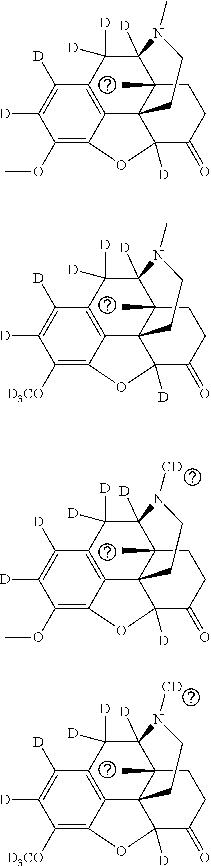

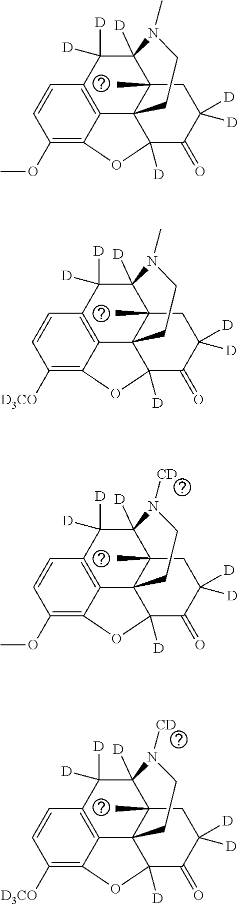

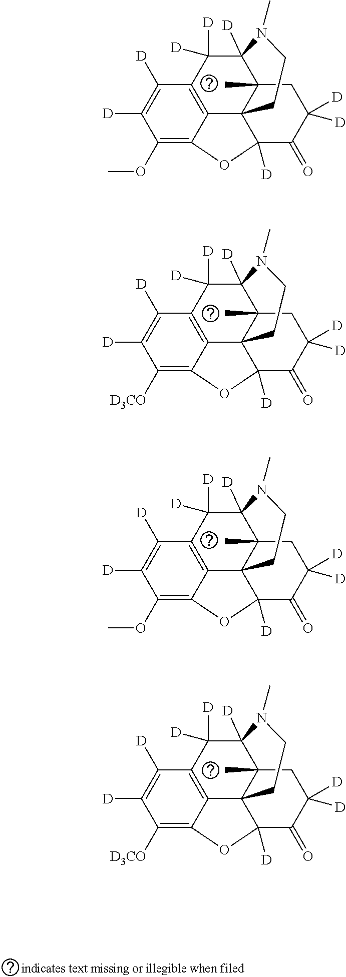

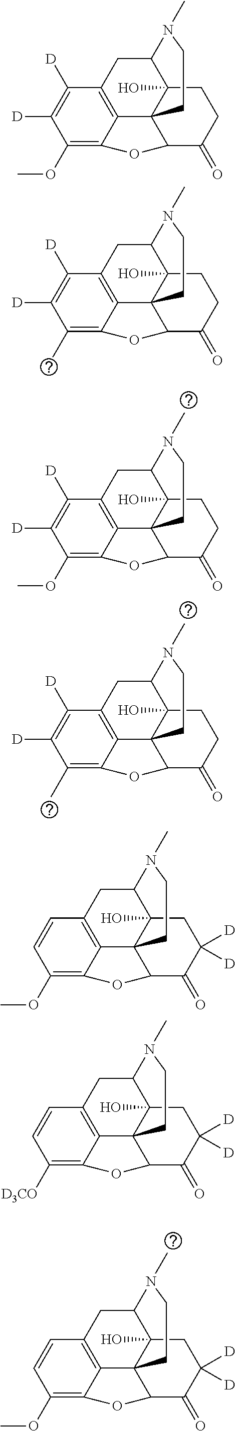

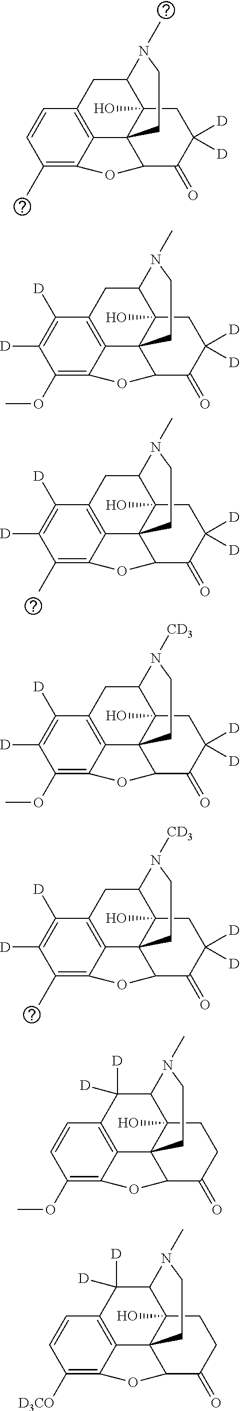

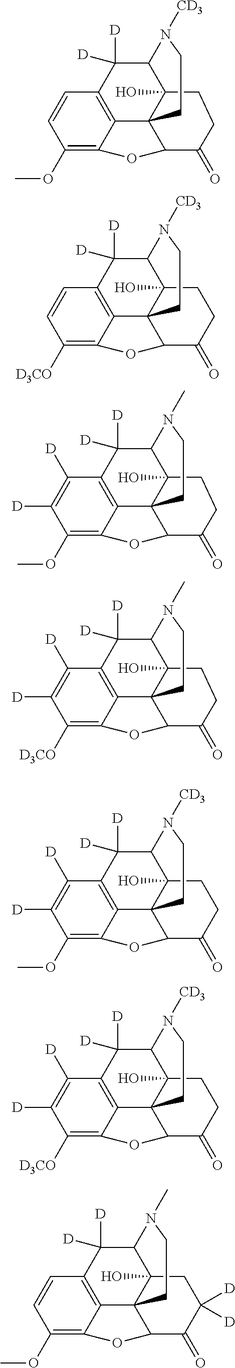

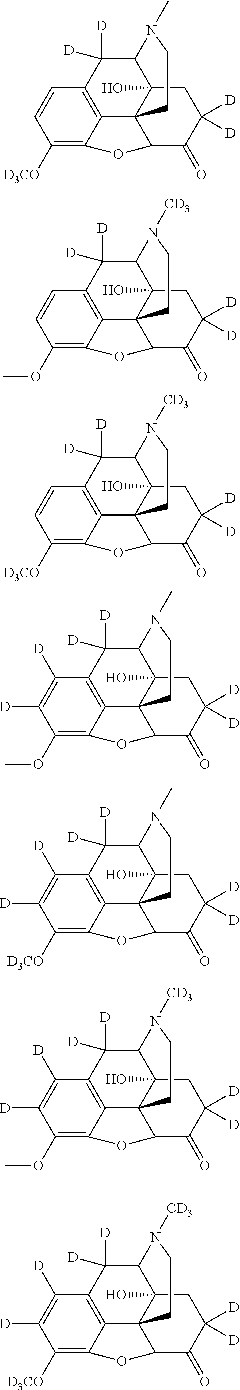

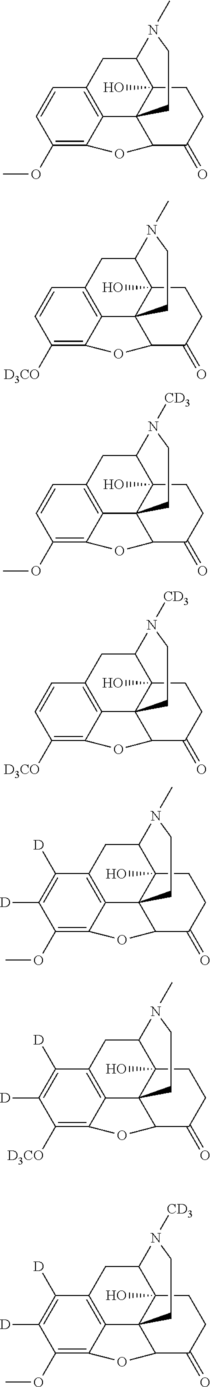

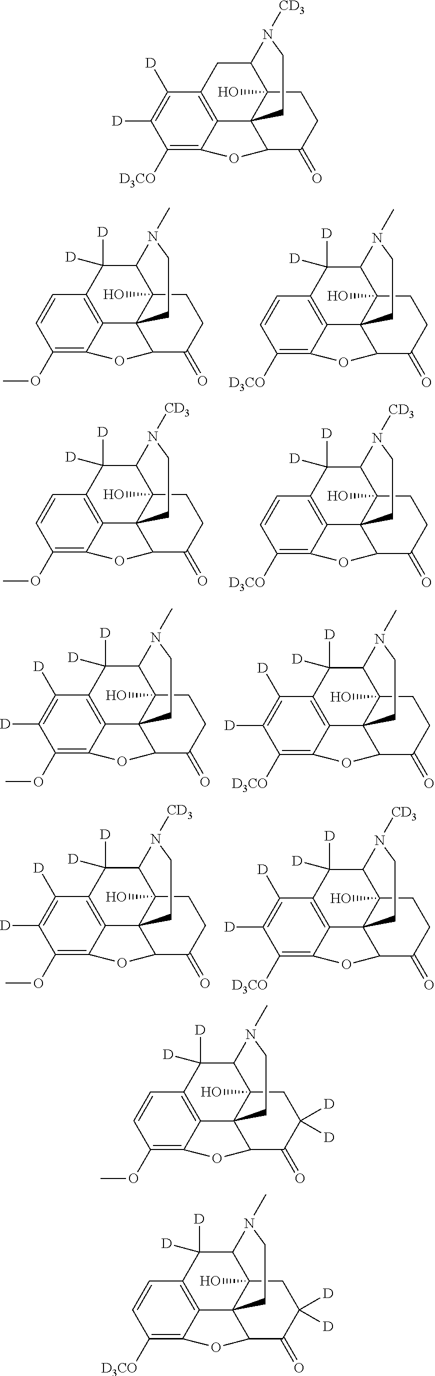

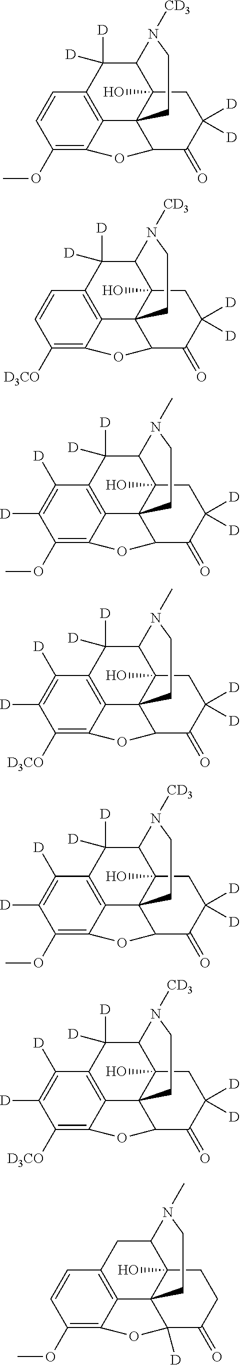

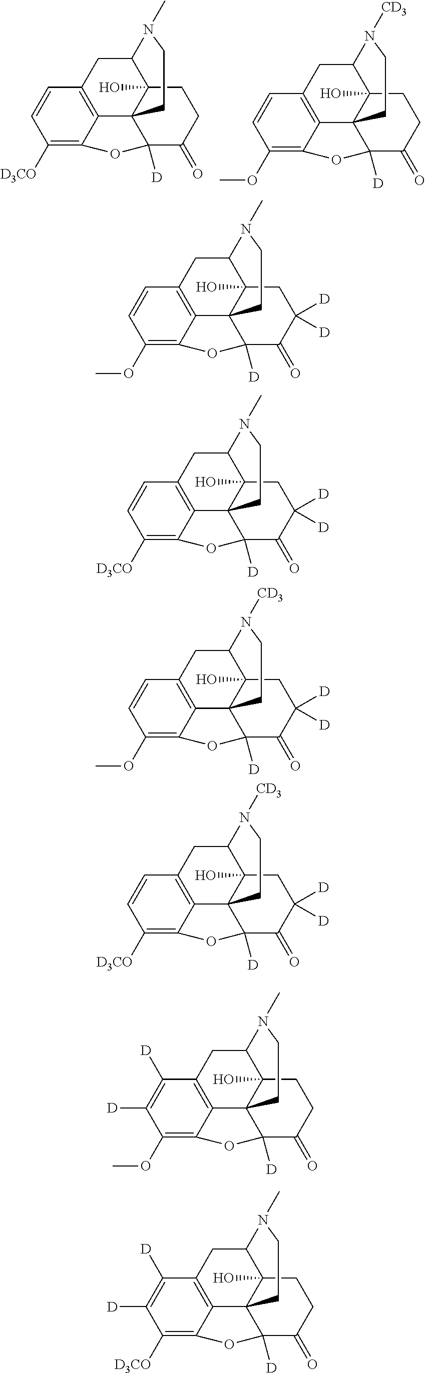

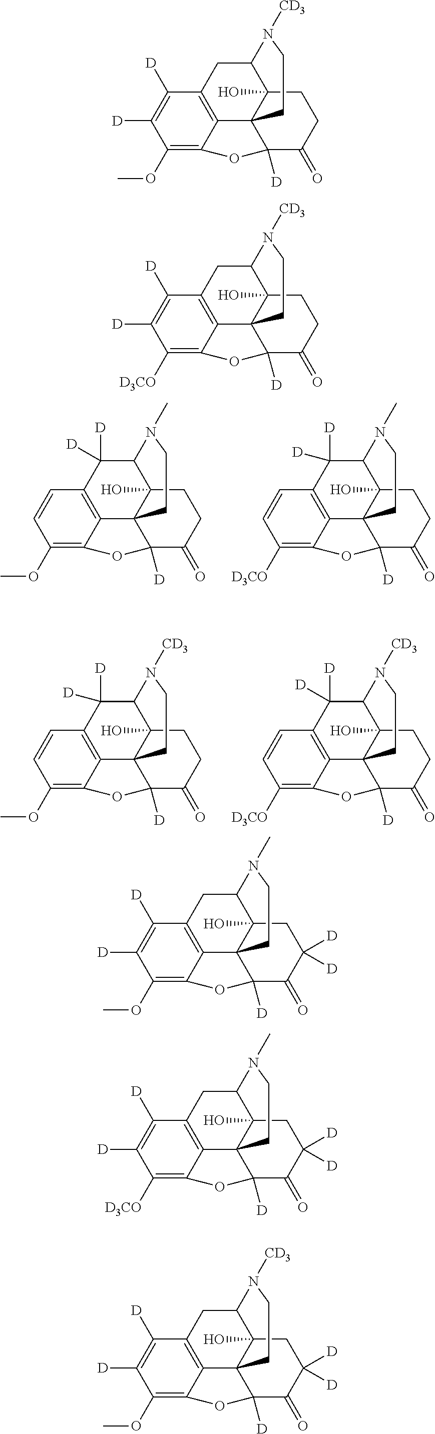

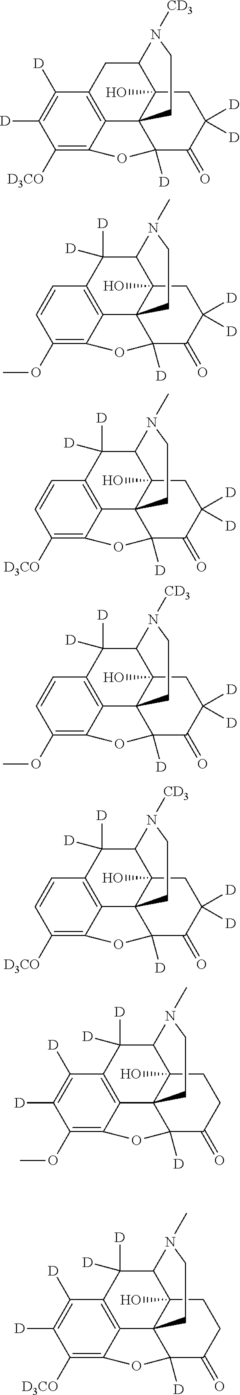

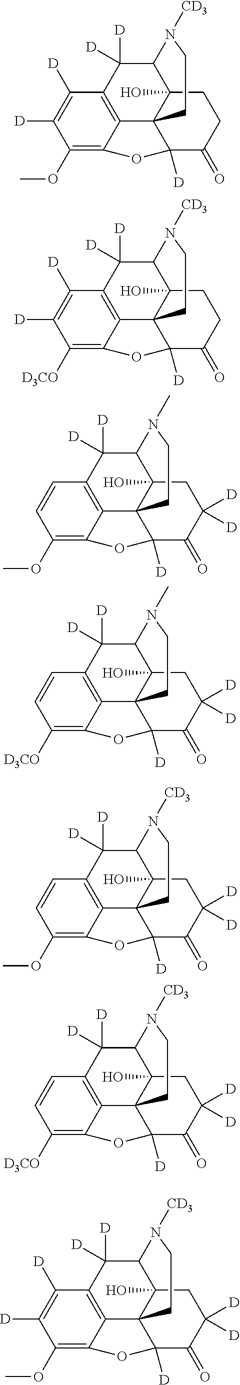

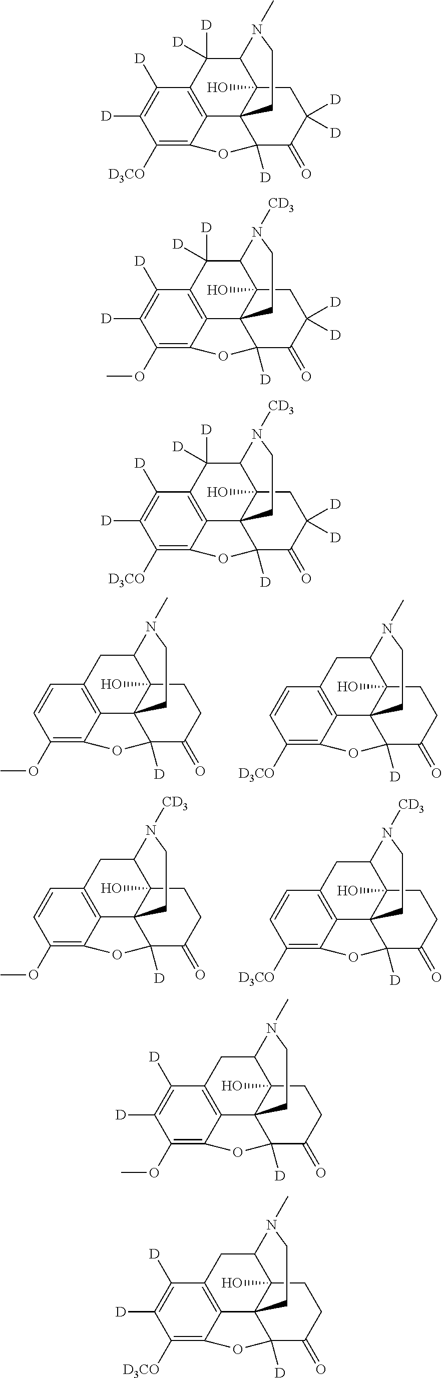

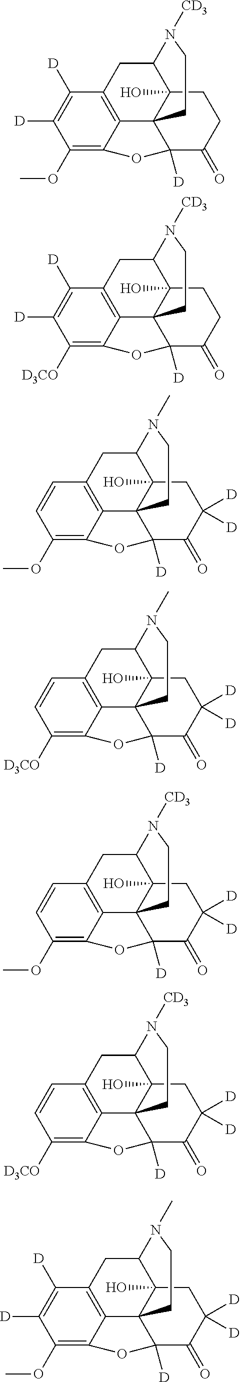

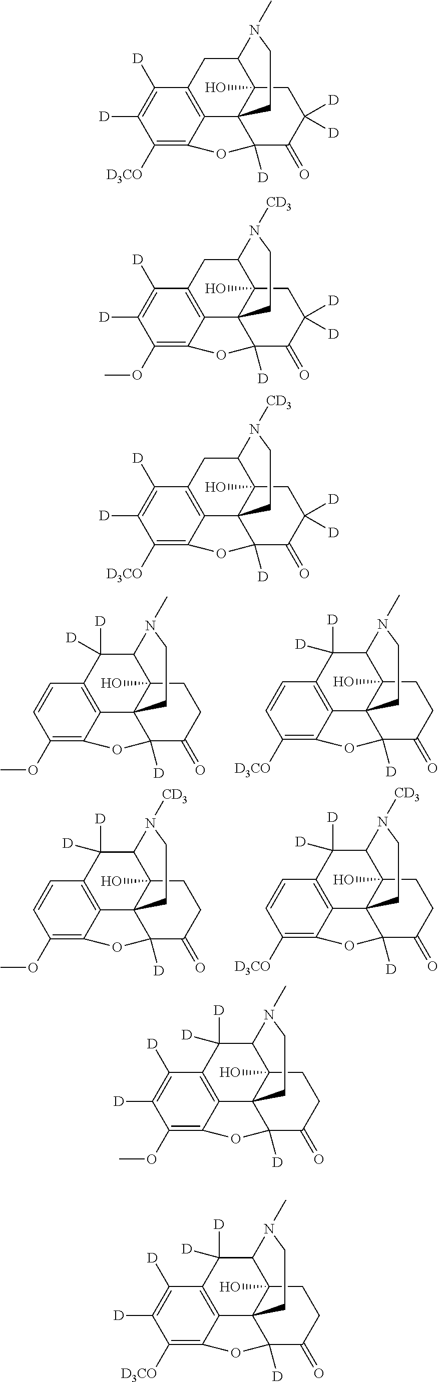

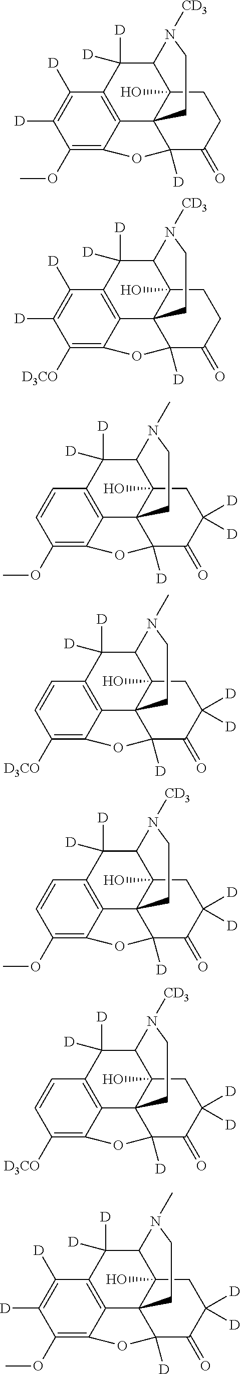

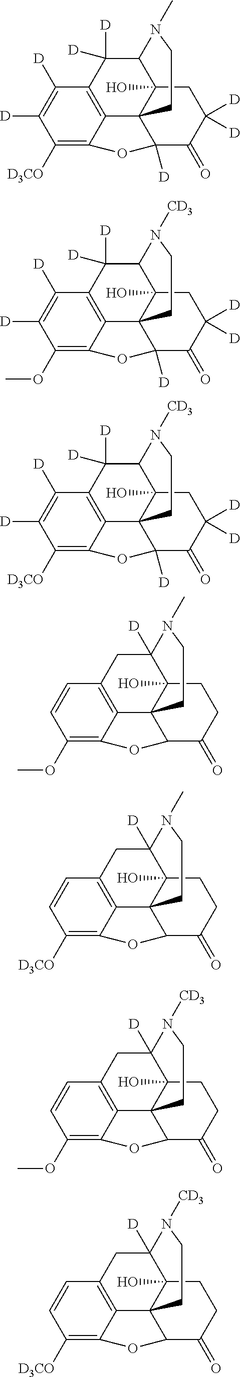

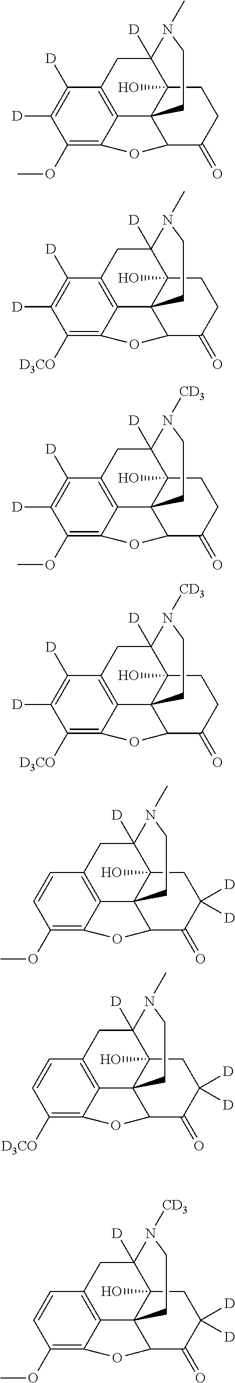

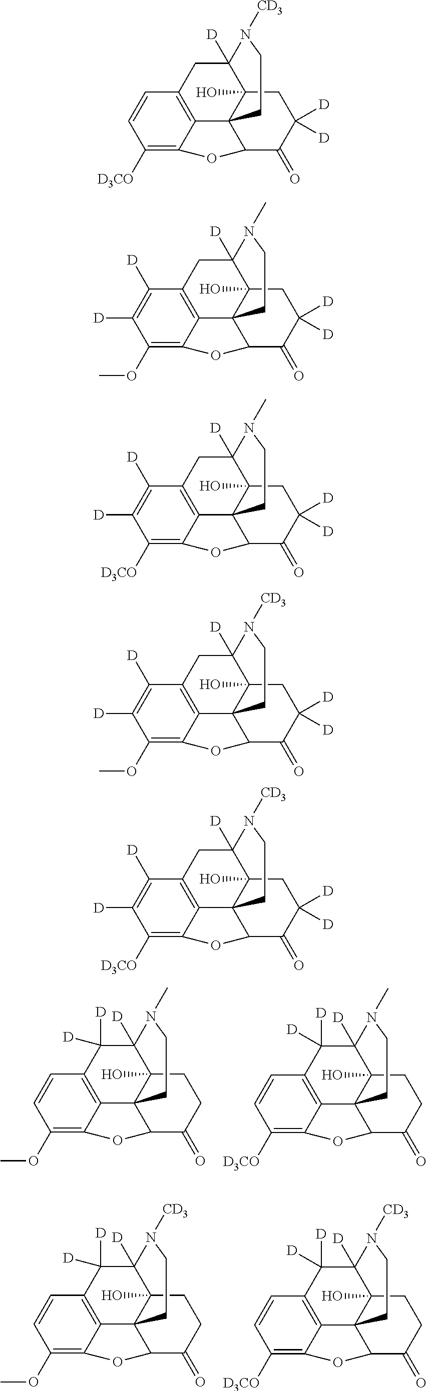

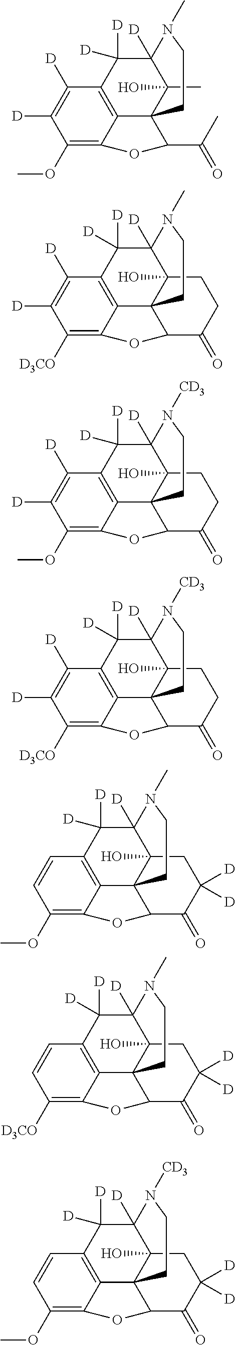

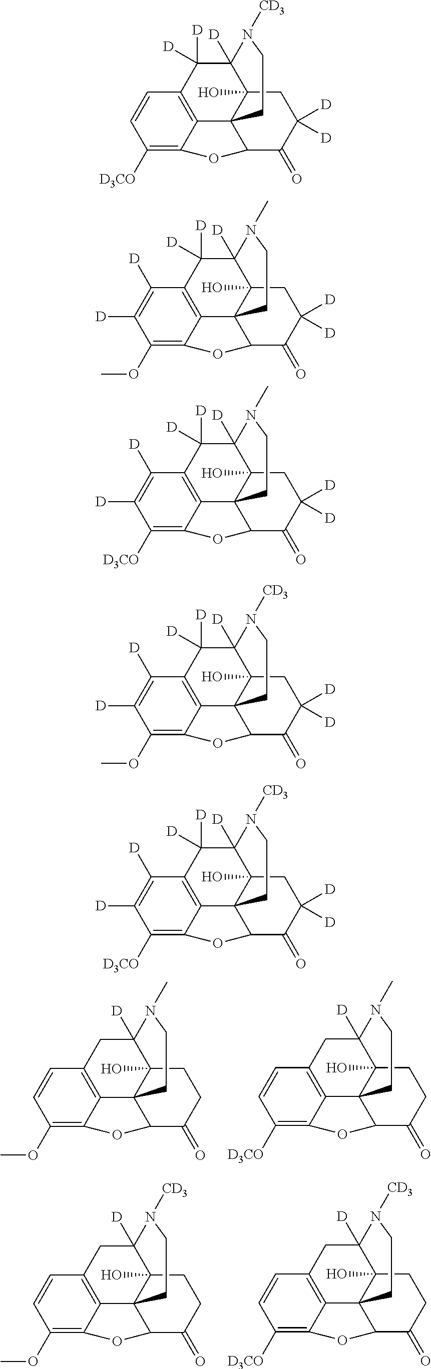

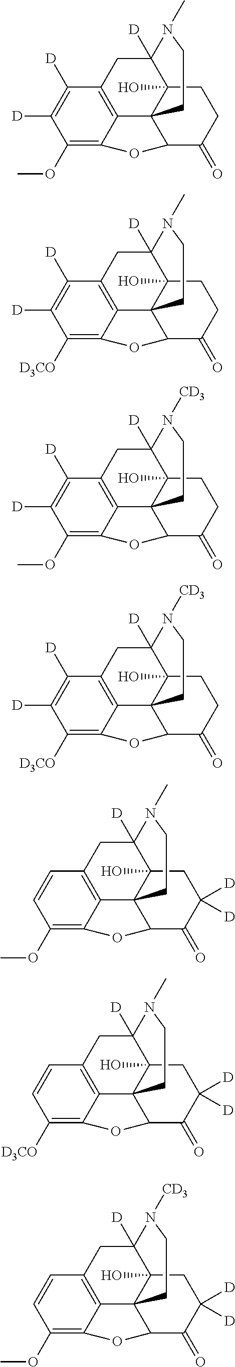

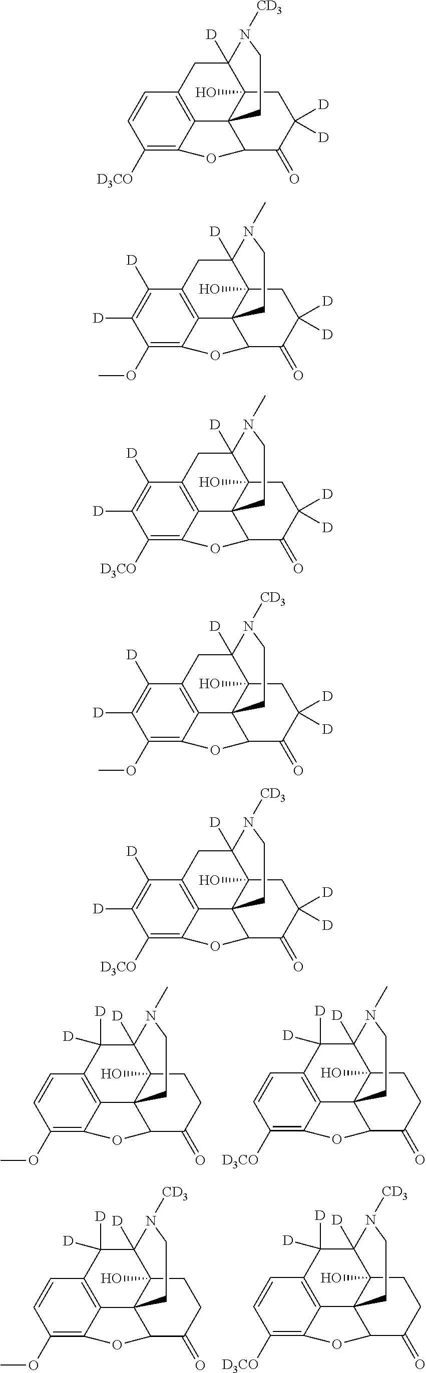

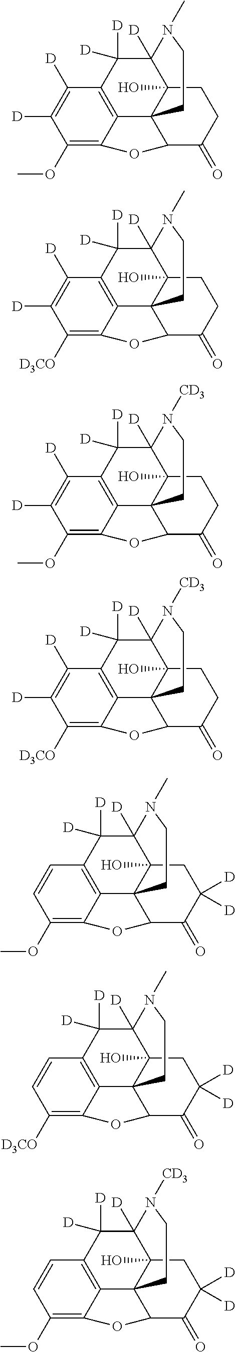

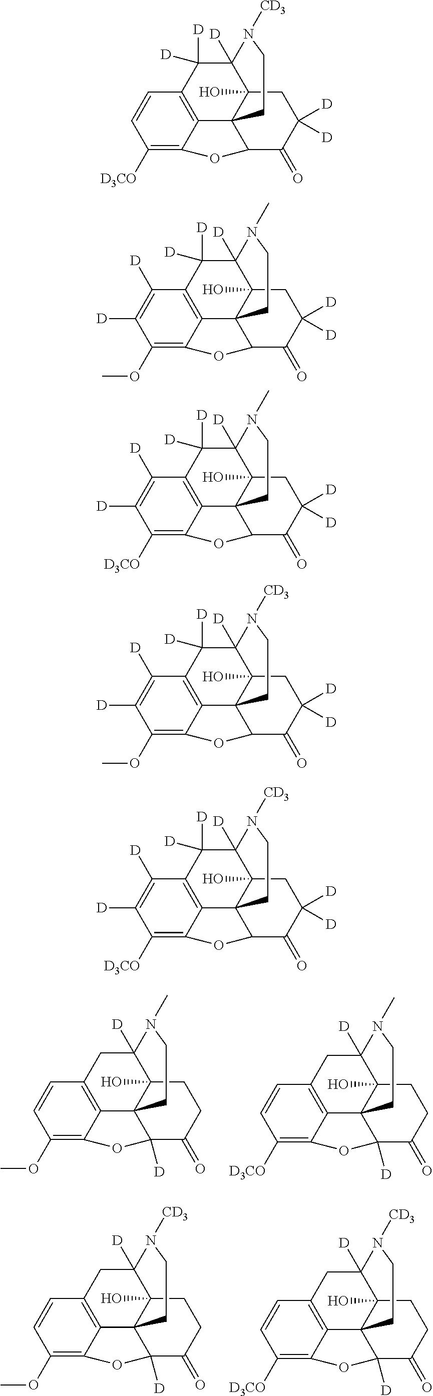

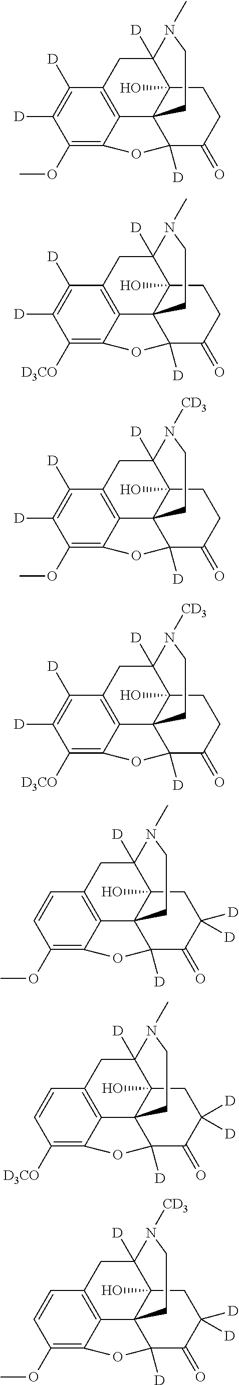

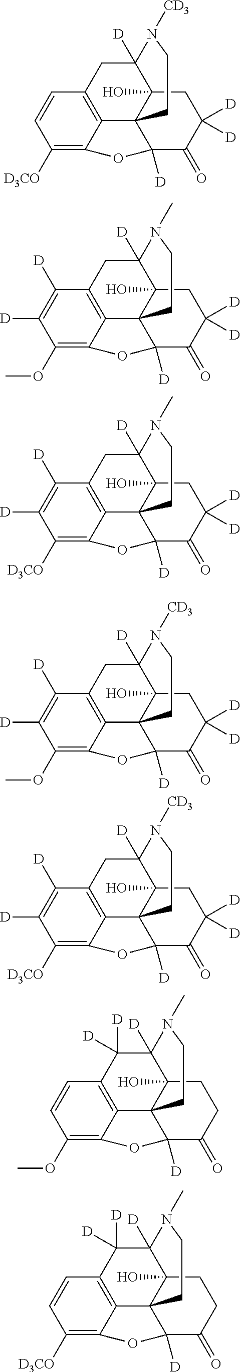

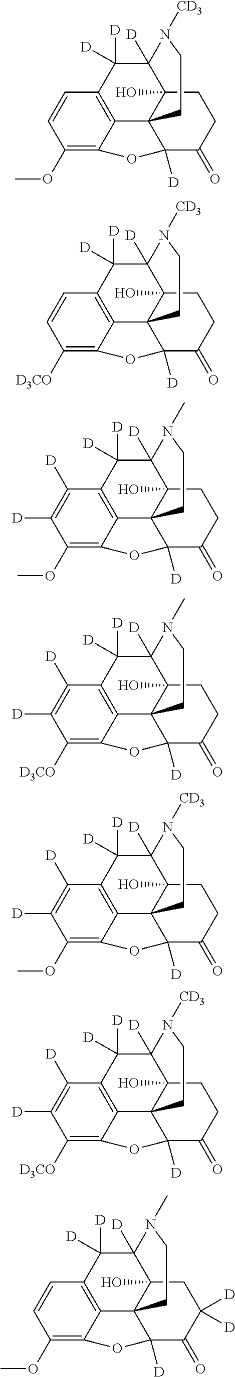

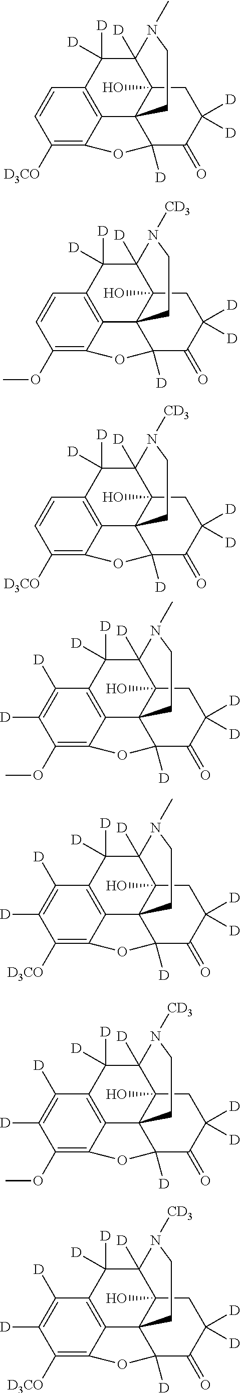

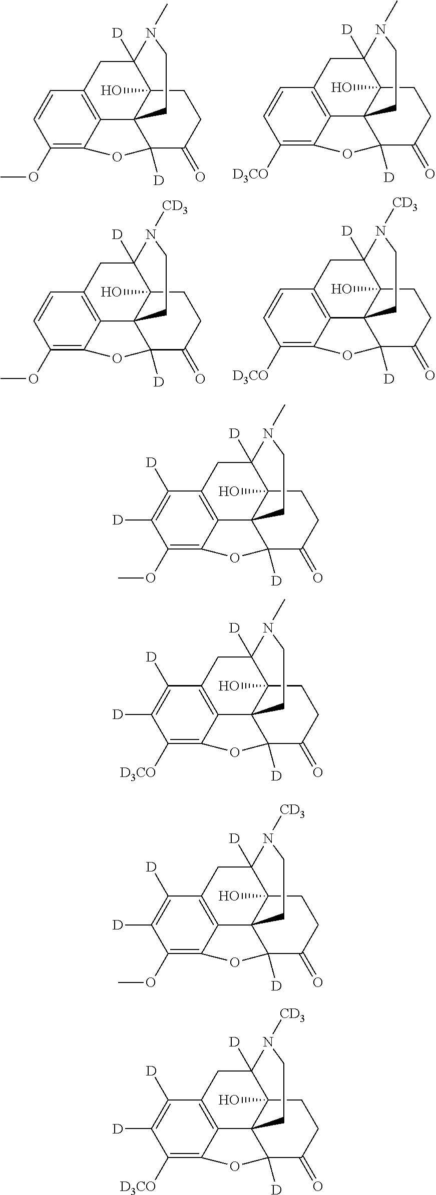

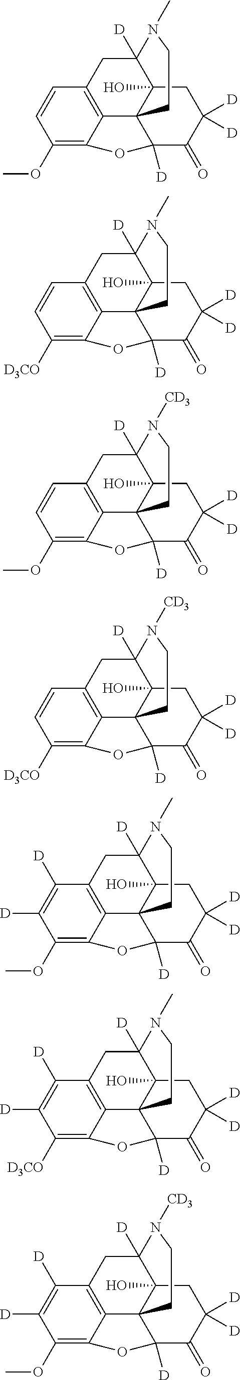

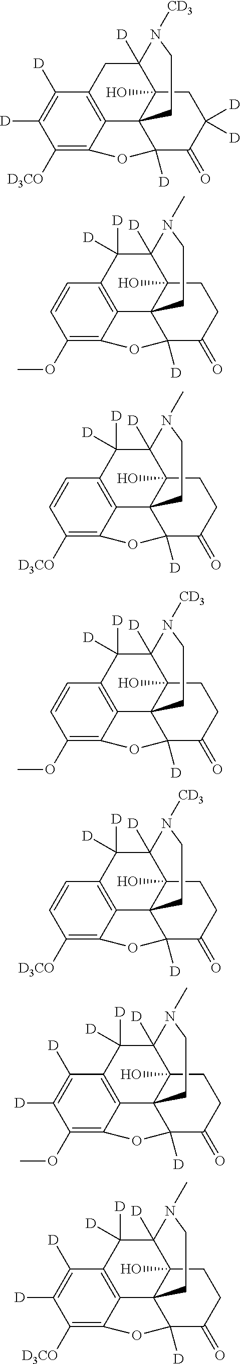

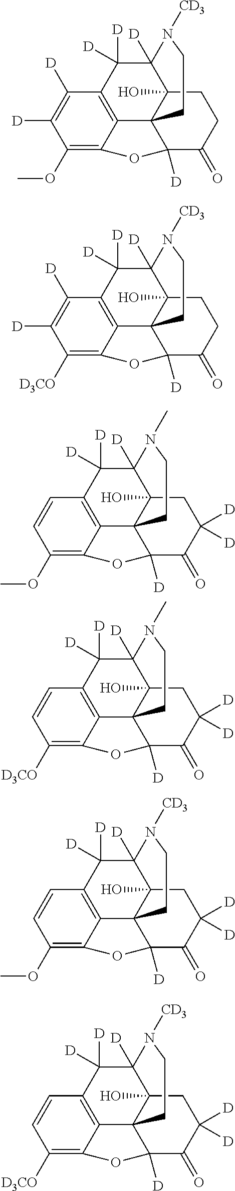

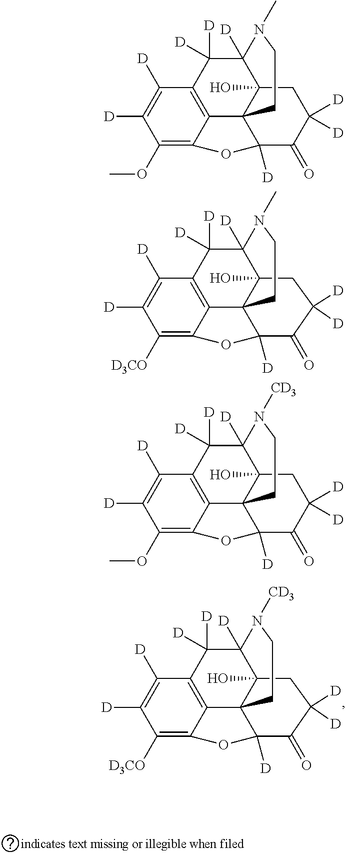

including enantiomers, pharmaceutically acceptable salts, solvates, or prodrugs thereof.

In an embodiment, the NMDA receptor antagonist may be selected from one of the following compounds according to Formula 219. Such compounds are described in WO 2006/017409, published Feb. 16, 2006 and corresponding to PCT/US2005/027160, filed Jul. 29, 2005, which are each incorporated by reference in their entirety herein. In the case of any conflict of terminology of Formula 219, these references incorporated by reference herein control.

In an embodiment, the NMDA receptor antagonist is a compound according to Formula 219:

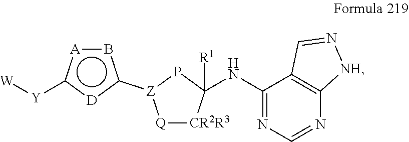

wherein W is aryl or heteroaryl, unsubstituted or substituted with 1-5 substituents independently selected from halogen and C1-6 alkyl optionally substituted with one or more substituents selected from halogen, hydroxy, and C1-4 alkoxy, C1-4 alkoxy, C3-6 cycloalkyl, cyano and N(R4)2;
Y is absent or is selected from C 1-3 alkyl optionally substituted with one or more substituents selected from halogen, hydroxyl and C 1-4 alkoxy, cyclopropyl and C(O);
Z is N and P and Q are C(R4)2, or Z is CR5 and P and Q are C(R5)2;
A, B and D are each independently selected from O, CR6, S and NR6;
R1, R4 and R6 are each independently selected from hydrogen or C 1-4 alkyl optionally substituted with one or more of halogen and hydroxyl; and
R2, R3; and R5 are each independently selected from hydrogen, halogen, hydroxyl, C 1-4 alkyl optionally substituted with one or more substituents selected from halogen and hydroxyl, C 1-4 alkoxy, cyano and N(R4)2;
   and pharmaceutically acceptable salts and individual enantiomers and stereoisomers thereof.

In an embodiment, the NMDA receptor antagonist is a compound selected from:

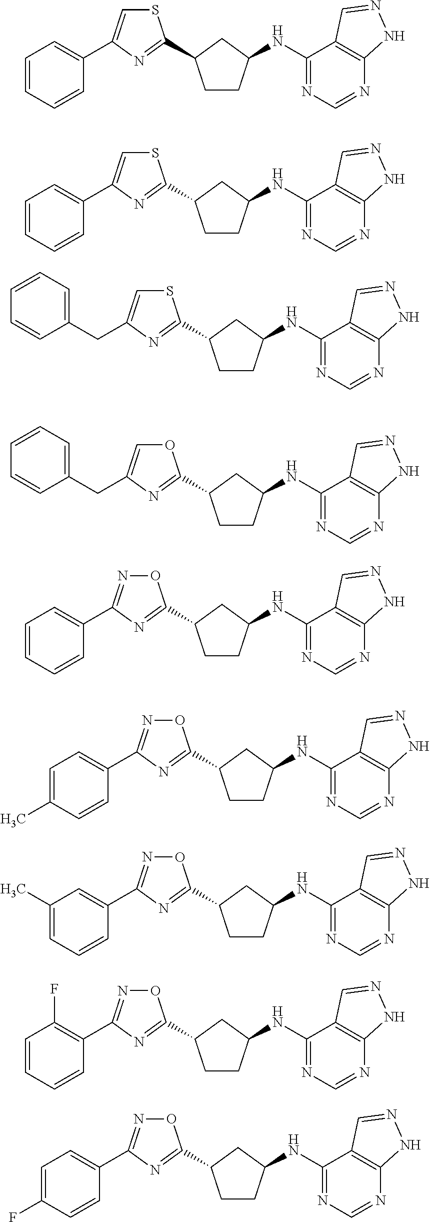

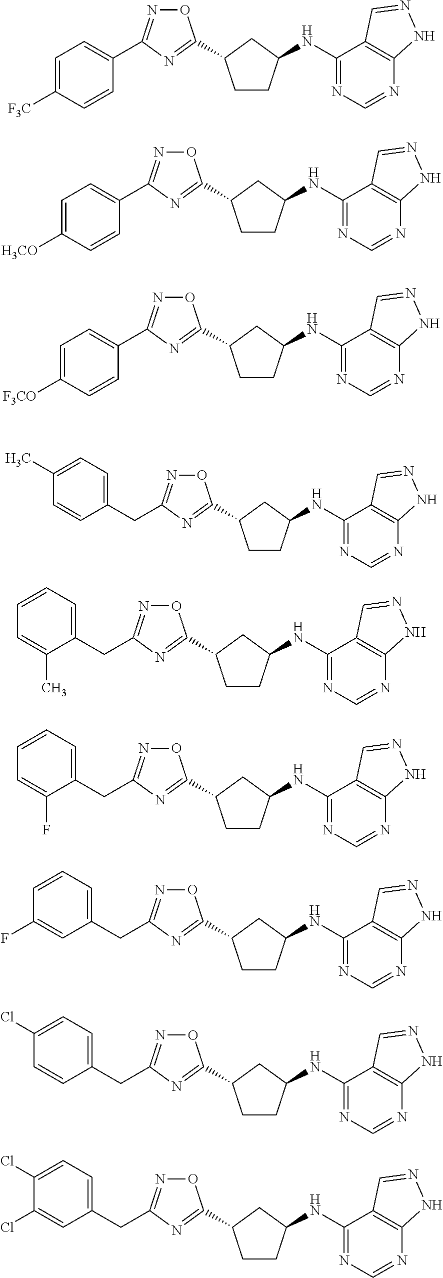

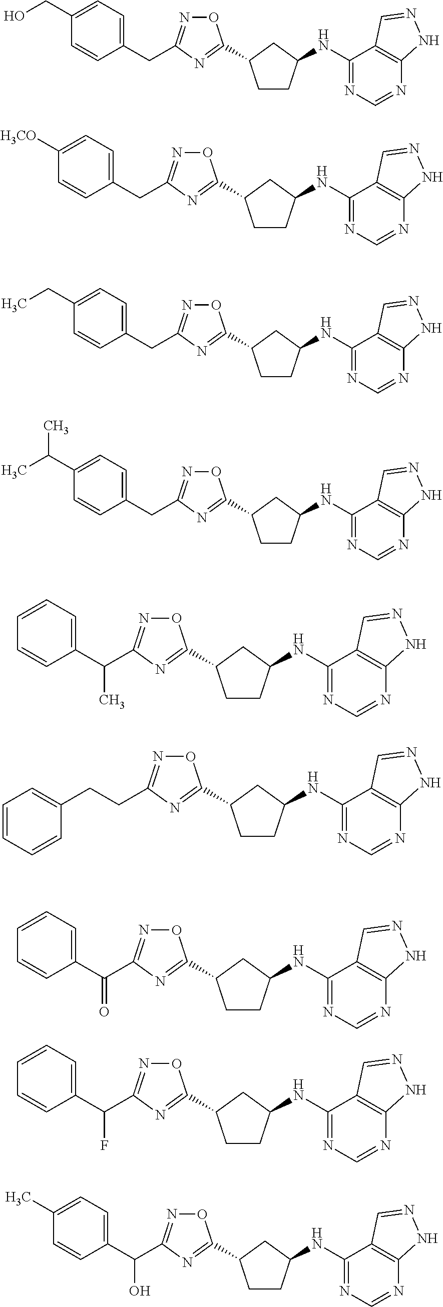

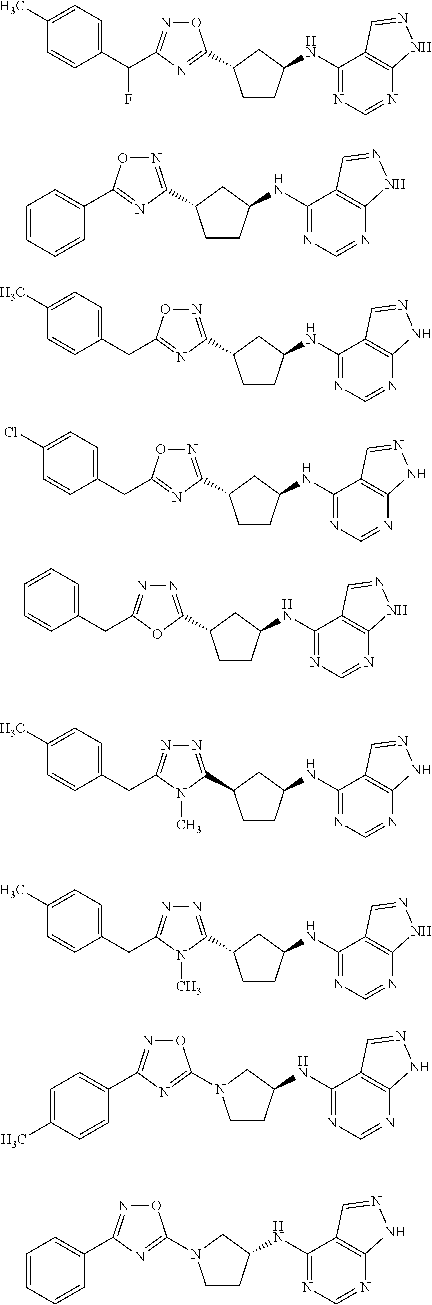

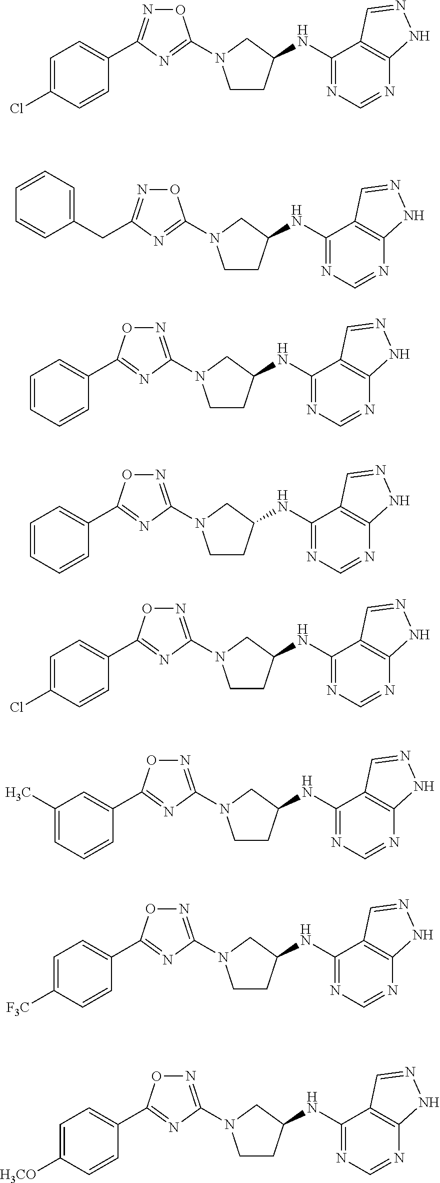

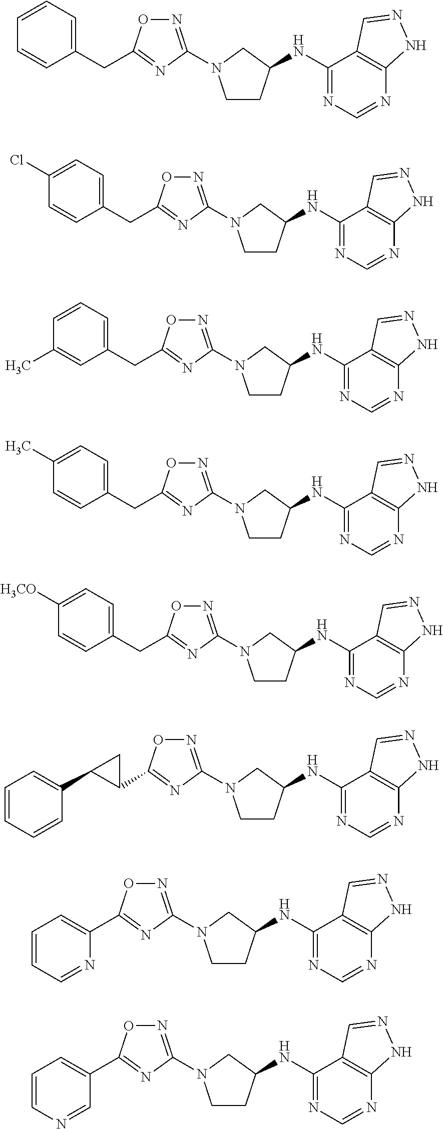

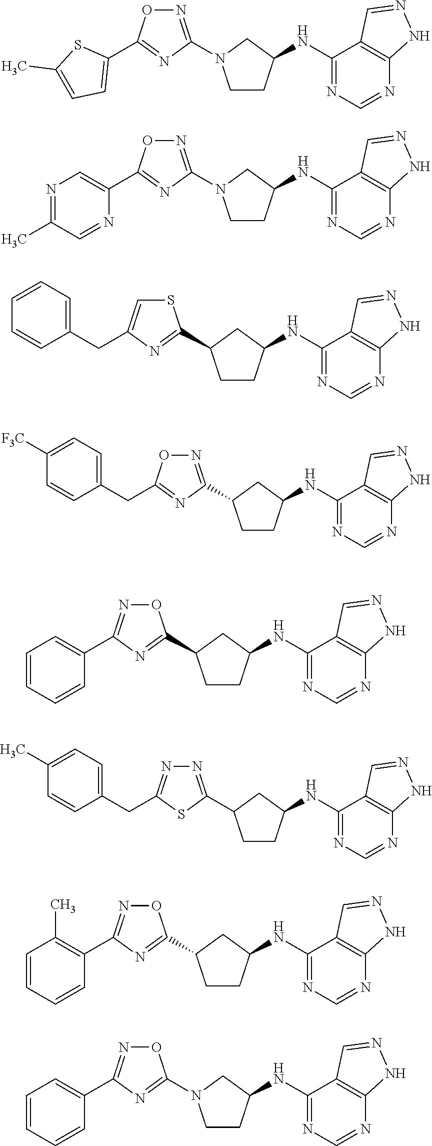

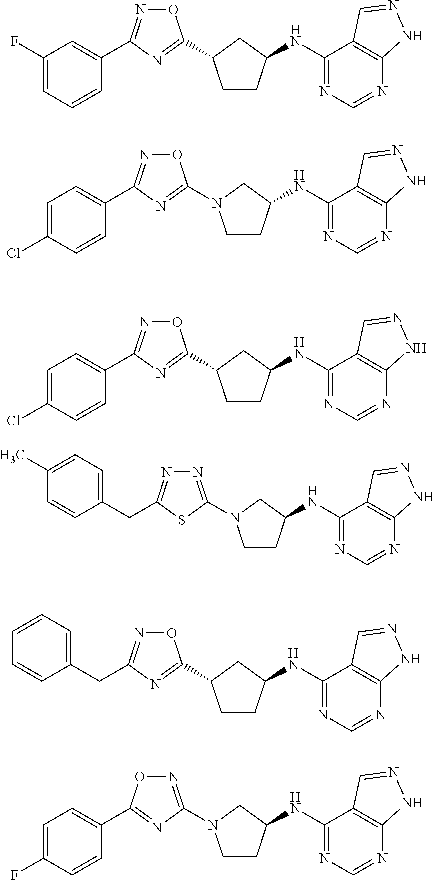

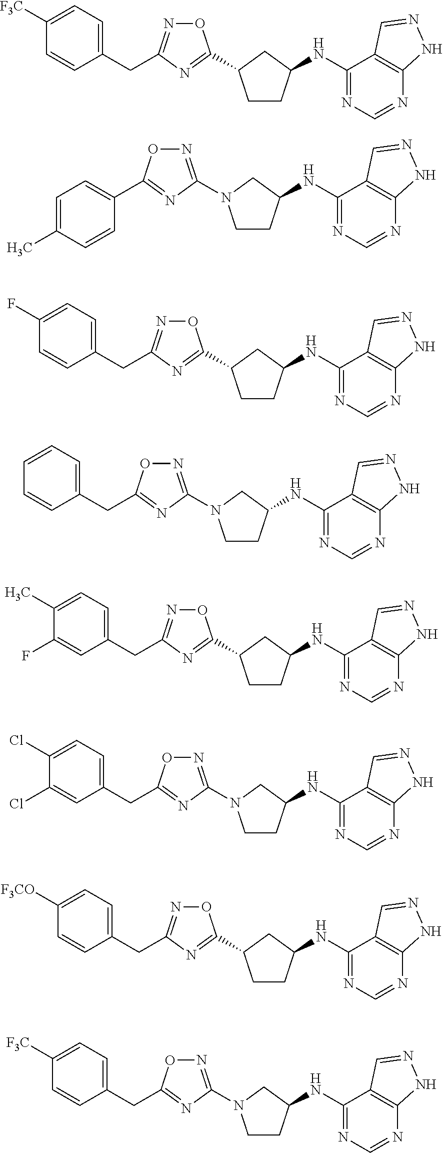

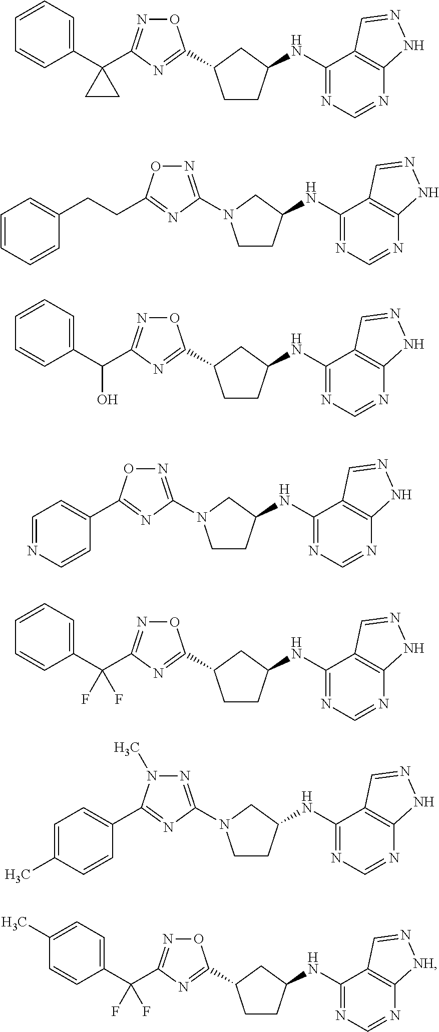

and pharmaceutically acceptable salts and individual enantiomers and stereoisomers thereof.

### Formula 220

In an embodiment, the NMDA receptor antagonist may be selected from one of the following compounds according to Formula 220. Such compounds are described in WO 2003/097637, published Nov. 27, 2003 and corresponding to PCT/EP2003/005151, filed May 16, 2003, which are each incorporated by reference in their entirety herein. In the case of any conflict of terminology of Formula 220, these references incorporated by reference herein control.

In an embodiment, the NMDA receptor antagonist is a compound according to Formula 220a or 220b:

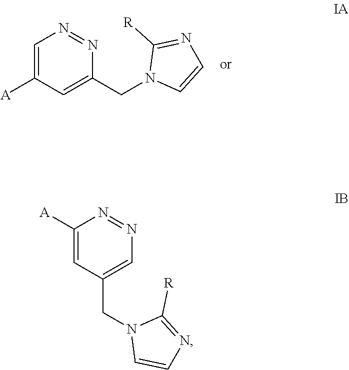

wherein A is an unsubstituted or substituted cyclic group; and R is a hydrogen or lower alkyl; and pharmaceutically acceptable salts thereof.

In an embodiment, A is the group:

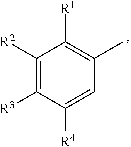

and wherein R1, R2, R3, and R4 are independently hydrogen, halogen, CF3, CHF2, C(CH3) F2, C3-C6 cycloalkyl, lower alkoxy, lower alkyl, OCF3, or phenyl; or A is

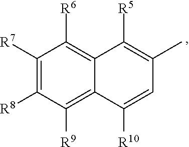

and R5, R6, R7, R8, R9, R10 are independently hydrogen, halogen, lower alkyl, lower alkoxy, or CHF2; or A is

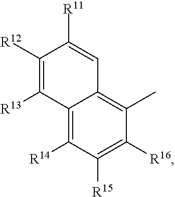

and R11, R12, R13, R14, R15, and R16 are independently hydrogen, halogen, lower alkoxy, or lower alkyl.

In further embodiments, A is

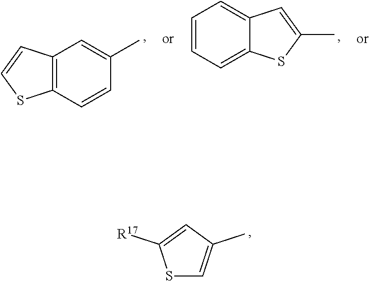

wherein R17 is hydrogen or CHF2; or A is

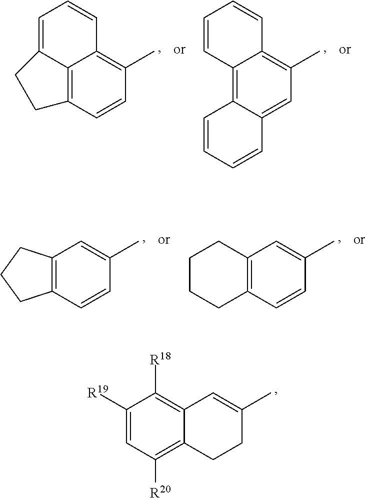

wherein R18, R19, and R20 are independently hydrogen, lower alkyl or lower alkoxy.

In further embodiments, the compound is:
5-(3-chloro-4-fluoro-phenyl)-3-(2-methyl-imidazol-1-yl-methyl)-pyridazine,
3-(2-methyl-imidazol-1-yl-methyl)-5-(3-trifluoromethyl-phenyl)-pyridazine,
5-(3-difluoromethyl-4-fluoro-phenyl)-3-(2-methyl-imidazol-1-yl-methyl)-pyridazine,
5-[3-(1,1-difluoro-ethyl)-phenyl]-3-(2-methyl-imidazol-1-yl-methyl)-pyridazine,
5-[3-(1,1-difluoro-ethyl)-4-fluoro-phenyl]-3-(2-methyl-imidazol-1-yl-methyl)-pyridazine,
5-(4-fluoro-3-methyl-phenyl)-3-(2-methyl-imidazol-1-yl-methyl)-pyridazine,
5-(4-chloro-3-methyl-phenyl)-3-(2-methyl-imidazol-1-yl-methyl)-pyridazine,
5-[3-(1,1-difluoro-ethyl)-5-fluoro-phenyl]-3-(2-methyl-imidazol-1-yl-methyl)-pyridazine,
5-(3-difluoromethyl-4-fluoro-phenyl)-3-(2-ethyl-imidazol-1-ylethyl)-pyridazine or
5-(3-cyclopropyl-4-fluoro-phenyl)-3-(2-methyl-imidazol-1-yl-methyl)-pyridazine.
5-benzo[b]thiophen-5-yl-3-(2-methyl-imidazol-1-yl-methyl)-pyridazine.
   , or
5-(3,4-dihydro-naphthalen-2-yl)-3-(2-methyl-imidazol-1-yl-methyl)-pyridazine hydrochloride.

### Formula 221

In an embodiment, the NMDA receptor antagonist may be selected from one of the following compounds according to Formula 221. Such compounds are described in US 2002/0072485, published Jun. 13, 2002, corresponding to application Ser. No. 09/969,354, filed Oct. 2, 2001, which are each incorporated by reference in their entirety herein. In the case of any conflict of terminology of Formula 221, these references incorporated by reference herein control.

In an embodiment, the NMDA receptor antagonist is a compound according to Formula 221:

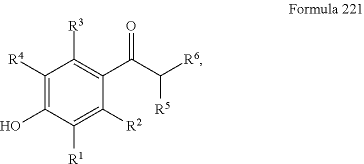

or a pharmaceutically acceptable acid addition salt thereof, wherein:
(a) R2 and R5 are taken separately and R1, R2, R3 and R4 are each independently hydrogen, (C1-C6)alkyl, halo, CF3, OH or OR7 and R5 is methyl or ethyl; or
(b) R2 and R5 are, taken together,

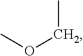

thereby forming a chroman-4-ol ring, and R1, R3 and R4 are each independently hydrogen, (C1-C6)alkyl, halo, CF3, OH or OR7;
R6 is

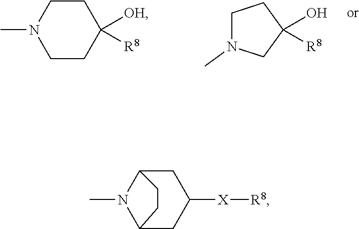

R7 is methyl, ethyl, isopropyl or n-propyl;
R8 is phenyl optionally substituted with up to three substituents independently selected from the group consisting of (C1-C6)alkyl, halo and CF3;
X is O, S or (CH2)n; and
n is 0, 1, 2, or 3.

In an embodiment, the NMDA receptor antagonist is selected from:
(+)-(1S, 2S)-1-(4-hydroxy-phenyl)-2-(4-hydroxy-4-phenylpiperidino)-1-propanol; a pharmaceutically acceptable acid addition salt thereof;
(1S, 2S)-1-(4-hydroxy-3-methoxyphenyl)-2-(4-hydroxy-4-phenylpiperidino)-1-propanol; a pharmaceutically acceptable acid addition salt thereof;
(3R,4S)-3-(4-(4-fluorophenyl)-4-hydroxypiperidin-1-yl)-chroman4,7-diol;
   a pharmaceutically acceptable acid addition salt thereof; and
(1R*, 2R*)-1-(4-hydroxy-3-methylphenyl)-2-(4-(4-fluorophenyl)-4-hydroxypiperidin-1-yl)-propan-1-ol-mesylate.

### Formula 222

In an embodiment, the NMDA receptor antagonist may be selected from one of the following compounds according to Formula 222. Such compounds are described in US 2002/0077352, published Jun. 20, 2002, corresponding to U.S. application Ser. No. 09/922,535, filed Aug. 3, 2001, which are each incorporated by reference in their entirety herein. In the case of any conflict of terminology of Formula 222, these references incorporated by reference herein control.

In an embodiment, the NMDA receptor antagonist is selected from:

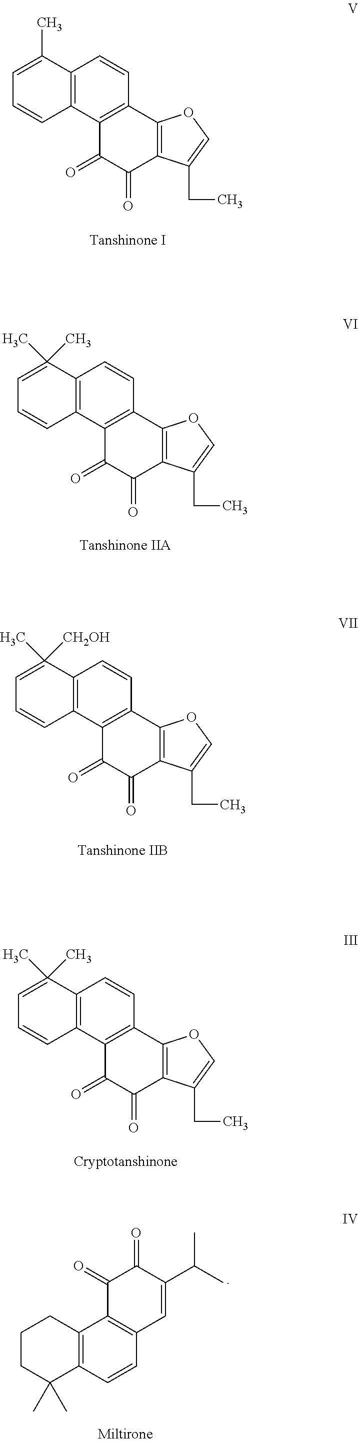

In further embodiments, the NMDA receptor antagonist is:

### Formula 223

In an embodiment, the NMDA receptor antagonist may be selected from one of the following compounds according to Formula 223. Such compounds are described in U.S. Pat. No. 6,794,402, issued Sep. 21, 2004, and PCT/US2001/014763, filed May 8, 2001 and corresponding to WO/2001/092239, published Dec. 6, 2001, which are each incorporated by reference in their entirety herein. In the case of any conflict of terminology of Formula 223, these references incorporated by reference herein control.

In an embodiment, the NMDA receptor antagonist is a bicycle-substituted cyclohexylamine.

In an embodiment, the NMDA receptor antagonist is a compound of Formula 223:

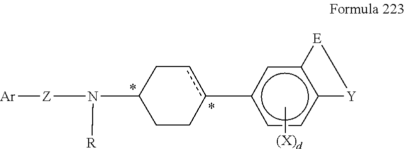

wherein:
Ar is substituted 1 to 3 times or unsubstituted aryl or substituted 1 to 3 times or unsubstituted heteroaryl, which heteroaryl is from 5 to 14 atoms having from 1 to 2 heteroatoms selected from N, O, and S wherein the substituents are selected from the groups F, Cl, Br, I, OH, NH2, SH, CN, NO₂, OCH₃, OC(O) CH₃, CF₃, OCH₂CH₂OH, NHC(O) CH₃, NHCH₃, or N(CH₃)₂;

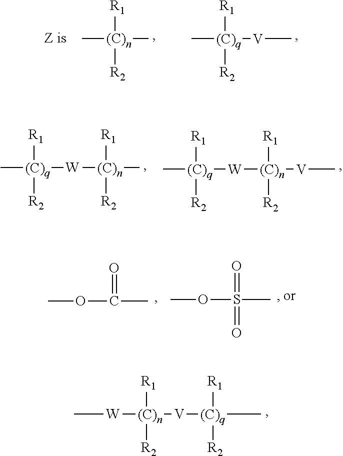

wherein V is —(CH₂)ₙ-,

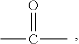

—S(O)—, or —S(O)₂-; W is —(CH₂)ₙ-,

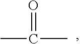

—S(SO)—, —S(O)2-, —O—, —S—, —C≡C—, or entgegen or zusammen —CH(R1)=CH(R2)-, d is an integer from 0 to 2;
n is an integer from 1 to 6;
q is an integer from 0 to 6;
R1 and R2 are independently selected from the group consisting of hydrogen, alkyl, OH, hydroxyalkyl, aminoalkyl, aralkyl, or N(R4) (R5) wherein R4 and R5 are independently selected from hydrogen, alkyl, aralkyl, heteroaryl, heteroaralkyl, aminoalkyl, hydroxyalkyl, and thioalkyl;
R is hydrogen, alkyl, C(O)R6, C(O)OR6, C(O) NHR6, -alkyl-C(O) NH2, aralkyl, (C3-C7 cycloalkyl)-alkyl, hydroxyalkyl, aminoalkyl, amino (hydroxy)alkyl, carboxyalkyl, heteroaralkyl, alkenylalkyl, or OH wherein R6 is alkyl or aralkyl;
X is independently selected from hydrogen or an electron withdrawing group;
-E-Y— is —CH—CH—N(H)—;
—(CH2)2-N(H),
—CH═N—N(H),
—C(O)—CH2-N(H)—,
CH2-C(O)—N(H)—,
—CH2-S(O)—N(H),
—CH2-S(O)2-N(H)—,
—CH═CH—CH(OH),
—(CH2)2-CH(OH)—,
—C(O)—CH2-C(O)—,
—C(O)—NH—C(O)—,
—N═CH—N(H)—,
—N(H)—C(O)—N(H)—,
—O—C(O)—NH—,
—S—C(O)—NH—,
—O—N—CH(OH),
—S—N═CH(OH)—,
—N═N—N(H)—,
—CH═CH—CH═C(OH)—,
—(CH2)3-CH(OH)—,
—(CH2)2-S(O)—N(H)—,
—(CH2)2-S(0)2-N(H),
—CH═CH—C(O) N(H),
—C(O)—NH—N═C(OH)—,
—CH═N—NH—C(O),
—CH═N(O)—N═C(OH)—,
—N(H)—C(O)—N(H) C(O)—,
—N═CH—C(O)—NH—,
—O—CH2-C(O)—NH—,
—S—CH2-C(O)—NH—, or
—N(H)—C(O)—C(O)—N(H)—,
- - - denotes a single or double bond; and
   * denotes cis or trans or a mixture thereof.

In further embodiments, the NMDA receptor antagonist is a compound selected from:
6-[trans-4-(3-Phenylpropylamino)cyclohexyl]-3H-benzoxazol-2-one;
6-{trans-4-[2-(4-Fluorophenoxy)ethylamino]cyclohexyl}-3H-benzoxazol-2-one;
6-[trans-4-(2-Phenoxyethylamino)cyclohexyl]-3H-benzoxazol-2-one;
6-{trans-4-[3-(4-Fluorophenyl)propylamino]cyclohexyl}-3H-benzoxazol-2-one;
6-{cis-4-[3-(4-Fluorophenyl)propylamino]cyclohexyl}-3H-benzoxazol-2-one;
6-[trans-4-(2-Phenylsulfanylethylamino)cyclohexyl]-3H-benzoxazol-2-one;
6-[cis-4-(2-Phenylsulfanylethylamino)cyclohexyl]-3H-benzoxazol-2-one;
6-[4-(3-Phenylpropylamino)cyclohex-1-enyl]-3H-benzoxazol-2-one;
6-{trans-4-[2-(4-Fluorophenylsulfanyl)ethylamino]cyclohexyl}-3H-benzoxazol-2-one;
6-{trans-4-[(R)-1-Methyl-3-phenylpropylamino]cyclohexyl}-3H-benzoxazol-2-one;
6-{trans-4-[(S)-1-Methyl-3-phenylpropylamino]cyclohexyl}-3H-benzoxazol-2-one;
6-{trans-4-[(1S,2S)-2-Hydroxy-2-(4-methoxyphenyl)-1-methylethylamino]cyclohexyl}-3H-benzoxazol-2-one;
6-{cis-4-[(1S,2S)-2-Hydroxy-2-(4-methoxyphenyl)-1-methylethylamino]-cyclohexyl}-3H-benzoxazol-2-one;
6-{trans-4-[(1S,2R)-2-Hydroxy-1-methyl-2-phenylethylamino]-cyclohexyl}-3H-benzoxazol-2-one;
6-[cis-4-(3-p-Tolylpropylamino)cyclohexyl]-3H-benzoxazol-2-one;
6-[trans-4-(3-p-Tolylpropylamino)cyclohexyl]-3H-benzoxazol-2-one;
6-[trans-4-(2-Benzenesulfinylethylamino)cyclohexyl]-3H-benzoxazol-2-one;
6-[trans-4-(2-Benzenesulfonylethylamino)cyclohexyl]-3H-benzoxazol-2-one;
6-{trans-4-[Methyl(3-phenylpropyl)amino]cyclohexyl}-3H-benzoxazol-2-one;
6-(trans-4-{[3-(4-Fluorophenyl)propyl]methylamino}cyclohexyl)-3H-benzoxazol-2-one;
6-(cis-4-{[3-(4-Fluorophenyl)propyl]methylamino}cyclohexyl)-3H-benzoxazol-2-one;
6-{trans-[4-(2-Methyl-3-phenylpropylamino)cyclohexyl]}-3H-benzoxazol-2-one
6-{trans-4-[Methyl(2-methyl-3-phenylpropyl)amino]cyclohexyl}-3H-benzoxazol-2-one;
6-{trans-[4-(3-Phenyl-prop-2-ynylamino)cyclohexyl]}-3H-benzoxazol-2-one;
6-{trans-4-[Methyl(3-phenylprop-2-ynyl)amino]cyclohexyl}-3H-benzoxazol-2-one;
6-{trans-4-[2-(4-Fluorophenylsulfanylethyl)methylamino]cyclohexyl}-3H-benzoxazol-2-one;
6-{trans-4-[3-(4-Trifluoromethylphenyl)propylamino]cyclohexyl}-3H-benzoxazol-2-one;
6-{trans-4-[Methyl-3-(p-tolylpropyl)amino]cyclohexyl}-3H-benzoxazol-2-one;
6-{trans-4-[Methyl(2-phenoxyethyl)amino]cyclohexyl}-3H-benzoxazol-2-one;
6-{trans-4-[Methyl-3-(4-tifluoromethylphenyl)propylamino]cyclohexyl}-3H-benzoxazol-2-one;
6-{trans-4-[3-(2,4-Difluorophenyl)propylamino]cyclohexyl}-3H-benzoxazol-2-one;
6-(trans-4-{[3-(2,4-Difluorophenyl)propyl]methylamino}cyclohexyl)-3H-benzoxazol-2-one;
6-(trans-4-{[3-(4-Isopropylphenyl)propyl]methylamino}cyclohexyl)-3H-benzoxazol-2-one;
6-(trans-4-{[3-(4-Isobutylphenyl)propyl]methylamino}cyclohexyl)-3H-benzoxazol-2-one;
6-(trans-4-{[Ethyl-[3-(4-fluorophenyl)propyl]amino}cyclohexyl)-3H-benzoxazol-2-one; 6-(trans-4-{Ethyl-[3-(4-trifluoromethylphenyl)propyl]amino}cyclohexyl)-3H-benzoxazol-2-one;
6-(trans-4-{Ethyl-[(R)-1-methyl-3-phenylpropyl]-amino}cyclohexyl)-3H-benzoxazol-2-one;
6-(trans-4-{Ethyl-[(S)-1-methyl-3-phenylpropyl]-amino}cyclohexyl)-3H-benzoxazol-2-one;
6-{trans-4-[[3-(4-Fluorophenyl)propyl]-(2-hydroxyethyl)amino]-yclohexyl}-3H-benzoxazol-2-one;
6-(trans-4-{Cyclopropylmethyl-[3-(4-fluorophenyl)propyl]amino}-cyclohexyl)-3H-benzoxazol-2-one;
6-(trans-4-{[3-(4-Fluorophenyl)propyl]furan-3-cylmethylamino}cyclohexyl)-3H-benzoxazol-2-one;
6-(trans-4-{Allyl-[3-(4-Fluorophenyl)propyl]amino}cyclohexyl)-3H-benzoxazol-2-one;
6-(trans-4-{[3-(4-Fluorophenyl)propyl]isobutylamino}cyclohexyl)-3H-benzoxazol-2-one;
6-(trans-4-{(2-Aminoethyl)-[3-(4-fluorophenyl)propyl]amino}-cyclohexyl)-3H-benzoxazol-2-one;
2-{[3-(4-Fluorophenyl)propyl]-[4-(2-oxo-2,3-dihydro-benzoxazol-6-yl)-cyclohexyl]amino}acetamide;
(S)-6-{trans-4-[1-(4-Fluorophenylsulfanyl)pentan-2-ylamino]cyclohexyl)}3H-benzoxazol-2-one;
(S)-6-{trans-4-[Ethyl(1-(4-fluorophenylsulfanyl)pentan-2-yl)amino]-cyclohexyl}-3H-benzoxazol-2-one;
6-[trans-4-(3-Phenylbutylamino)cyclohexyl]-3H-benzoxazol-2-one;
6-[trans-4-(Methyl-3-phenylbutylamino)cyclohexyl]-3H-benzoxazol-2-one;
6-{trans-4-[3-(4-Chlorophenyl)propylamino]cyclohexyl}-3H-benzoxazol-2-one;
6-(trans-4-{[3-(4-Chlorophenyl)propyl]methylamino}cyclohexyl)-3H-benzoxazol-2-one;
6-(trans-4-{[3-(2,4-Dichlorophenyl)propyl]methylamino}cyclohexyl)-3H-benzoxazol-2-one;
6-(trans-4-[3-(3,5-Difluorophenyl)propylamino]cyclohexyl}-3H-benzoxazol-2-one;
6-(trans-4-{[3-(3,5-Difluorophenyl)propyl]methylamino}cyclohexyl)-3H-benzoxazol-2-one;
6-(trans-4-[3-(3,4-Difluorophenyl)propylamino]cyclohexyl}-3H-benzoxazol-2-one;
6-(trans-4-{[3-(3,4-Difluorophenyl)propyl]methylamino}cyclohexyl)-3H-benzoxazol-2-one;
6-(trans-4-{[3-(2,3,4-Trifluorophenyl)propyl]methylamino}cyclohexyl)-3H-benzoxazol-2-one;
6-(4-{[3-(trans-4-Dimethylaminophenyl)propyl]methylamino}-cyclohexyl)-3H-benzoxazol-2-one;
6-(trans-4-{[3-(2,4,6-Trifluorophenyl)propyl]methylamino}cyclohexyl)-3H-benzoxazol-2-one;
6-(trans-4-[3-(2,4-Dimethylphenyl)propylamino]cyclohexyl}-3H-benzoxazol-2-one;
6-(trans-{4-[Methyl(2,4-dimethylphenylpropyl)amino]cyclohexyl})-3H-benzoxazol-2-one;
6-{trans-4-[3-(2-Chloro-4-fluorophenyl)propylamino]cyclohexyl}-3H-benzoxazol-2-one;
6-(trans-4-{[3-(2-Chloro-4-fluorophenyl)propyl]methylamino}-cyclohexyl)-3H-benzoxazol-2-one;
6-{trans-4-[3-(4-Chloro-2-fluorophenyl)propylamino]cyclohexyl}-3H-benzoxazol-2-one;
6-(trans-4-{[3-(4-Chloro-2-fluorophenyl)propyl]methylamino}-cyclohexyl)-3H-benzoxazol-2-one;
6-(trans-4-[3-(4-Fluoro-2-methylphenyl)propylamino]cyclohexyl}-3H-benzoxazol-2-one;
6-(trans-{4-[Methyl(4-fluoro-2-methylphenylpropyl)amino]cyclohexyl})-3H-benzoxazol-2-one;
6-{trans-4-[3-(3-Fluoro-2-methylphenyl)propylamino]cyclohexyl}-3H-benzoxazol-2-one;
6-(trans-4-{[3-(3-Fluoro-2-methylphenyl)propyl]methylamino}-cyclohexyl)-3H-benzoxazol-2-one;
6-{trans-4-[3-(2-Fluorophenyl)propylamino]cyclohexyl}-3H-benzoxazol-2-one;
6-(trans-4-{[3-(2-Fluorophenyl)propyl]methylamino}cyclohexyl)-3H-benzoxazol-2-one;
6-{trans-4-[3-(3-Fluorophenyl)propylamino]cyclohexyl}-3H-benzoxazol-2-one;
6-(trans-4-{[3-(3-Fluorophenyl)propyl]methylamino}cyclohexyl)-3H-benzoxazol-2-one;
6-{trans-4-[(3-Cyclohexylpropyl)amino]cyclohexyl}-3H-benzoxazol-2-one;
6-{trans-4-[(3-Cyclohexylpropyl)methylamino]cyclohexyl}-3H-benzoxazol-2-one;
6-{trans-4-[4-(3-Thiophen-3-yl)propylamino]cyclohexyl}-3H-benzoxazol-2-one;
6-[trans-4-{[3-(3-Thiophen-3-yl)propyl]methylamino}cyclohexyl)-3H-benzoxazol-2-one;
6-{trans-4-[Methyl(3-thiazol-2-ylpropyl)amino]cyclohexyl}-3H-benzoxazol-2-one;
6-(trans-4-{Methyl-[2-(methylphenylamino)ethyl]amino}cyclohexyl)-3H-benzoxazol-2-one;
N-(2-{Methyl-[4-(2-oxo-2,3-dihydrobenzoxazol-6-yl)cyclohexyl]amino}-ethyl)benzamide;
N-{2-[4-(2-Oxo-2,3-dihydrobenzoxazol-6-yl)cyclohexylamino]ethyl}-benzamide;
6-{trans-4-[3-(4-Fluorophenyl)propylamino]cyclohexyl}-3H-benzthiazol-2-one;
3-(4-Fluorophenyl)-N-methyl-N-[trans-4-(2-oxo-2,3-dihydrobenzoxazol-6-yl)cyclohexyl]propionamide;
6-(trans-4-{[3-(4-Fluorophenyl)propyl]methylamino}cyclohexyl)-3H-benzthiazol-2-one;
5-{trans-4-[3-(4-Fluorophenyl)propylamino]cyclohexyl}-1,3-dihydrobenzoimidazol-2-one;
5-(trans-4-{[3-(4-Fluorophenyl)propyl]methylamino}cyclohexyl)-1,3-dihydrobenzoimidazol-2-one;
6-(4-{[3-(4-Fluorophenyl)propyl]methyl-amino}cyclohexyl)-5-methyl-3H-benzoxazol-2-one; and
6-(trans-4-{[3-(4-Fluorophenyl)propyl]methylamino}cyclohexyl)-5-methoxy-3H-benzoxazol-2-one.

### Formula 224

In an embodiment, the NMDA receptor antagonist may be selected from one of the following compounds according to Formula 224. Such compounds are described in, which are each incorporated by reference in their entirety herein. In the case of any conflict of terminology of Formula 224, these references incorporated by reference herein control.

In an embodiment, the NMDA receptor antagonist is a compound according to Formula 224:

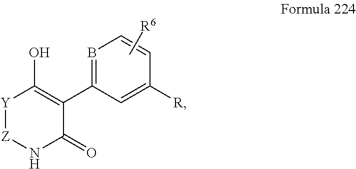

wherein B is CH or N,

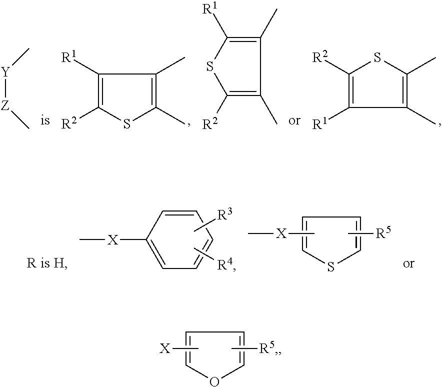

R1, R2, and R5 are independently H, A, or halogen,
R3 and R4 are independently H, A, OH, OA, halogen, CF3, NO2, NH2, NHA, N(A)2 or NHAc, R6 is H or halogen, X is —CH2-, —CO—, —O—, —NH—, —NA- or —S—, A is alkyl having 1-4 carbon atoms, Ac is an alkanoyl having 1-6 carbon atoms, or benzoyl and halogen is F, Cl, Br, or I, and pharmaceutically acceptable salts thereof.

In an embodiment, the NMDA receptor antagonist is 2-Chloro-3-methyl-4-hydroxy-5-phenyl-6,7-dihydrothieno-2,3-bipyridin-6-one

### Peptide NMDA Receptor Antagonists

In an embodiment, the NMDA receptor antagonist is a peptide agent. For ease of reference, the peptide NMDA receptor antagonists are classified under Formula numbers, each of which may refer to one or more different peptide sequences. Where a sequence homology percentage is recited, e.g. at least 80% sequence homology, it is contemplated that higher percentages are also contemplated. Specifically, where the term “at least 80% sequence homology” or, for short, “at least 80% homology” is recited, it should be understood that other percentages including at least 85%, at least 90%, at least 95%, at least 96%, at least 97%, at least 98%, and at least 99% sequence homology are expressly contemplated and included.

In various embodiments, the peptides may be useful as therapeutic agents. In further various embodiments, the peptides may be produced in vivo from a therapeutic agent containing a polynucleotide sequence encoding the peptide. In an embodiment, a polynucleotide sequence may be a DNA sequence or an RNA sequence. In an embodiment, the sequence may be delivered or introduced to the patient by a plasmid or vector. It should be appreciated that any known delivery technique or construct is contemplated.

### Formula 250

In an embodiment, the NMDA receptor antagonist may be selected from one of the following peptides according to Formula 250. Such peptides are described in U.S. Pat. No. 10,689,418, issued Jun. 23, 2020 and corresponding to application Ser. No. 16/065,078, filed Jun. 21, 2018, which are each incorporated by reference in their entirety herein. In the case of any conflict of terminology of Formula 250, these references incorporated by reference herein control.

In an embodiment, the NMDA receptor antagonist is a peptide comprising the amino acid sequence GEDDLQDNQDLIRDKSN, or a sequence having at least 80% sequence homology thereto.

In an embodiment, the NMDA receptor antagonist is a peptide comprising the amino acid sequence GEDDYQDAQDLIRDKSN, or a sequence having at least 80% sequence homology thereto.

In an embodiment, the NMDA receptor antagonist is a peptide comprising the amino acid sequence KLGMRSELQIDNDQDAD, or a sequence having at least 80% sequence homology thereto.

### Formula 251

In an embodiment, the NMDA receptor antagonist may be selected from one of the following peptides according to Formula 251. Such peptides are described in WO 2021/137245, published Jul. 8, 2021 corresponding to PCT/IL2021/050013, which are each incorporated by reference in their entirety herein. In the case of any conflict of terminology of Formula 251, these references incorporated by reference herein control.

In an embodiment, the NMDA receptor antagonist is a natural or modified conantokin-P peptide.

In an embodiment, the NDMA receptor antagonist is a peptide comprises one or more amino acid sequences according to:

| | |
|---|---|
| WT Con-P | GEγγHSKYQγCLRγIRVNKVQQγC |
| (Conus | |
| purpurascens) | |
| WT Con-G | GEγγLQγNQγLIRγKSN |
| (Conus | |
| geographus) | |
| WT Con-Pr3 | GEPγVAKWAγGLRγKAASN |
| (Conus parius) | |

or one or more amino acid sequences having at least 80% sequence homology thereto.

In an embodiment, the NMDA receptor antagonist comprises one or more amino acid sequences according to:

| | |
|---|---|
| Naked Con-P | GEEEHSKYQECLREIRYNKVQQEC |
| Ala-sub Con-P | GAAAHSKYQACERAIRVNKVQQAC |
| Asp-Sub Con-P | GDDDHSKYQDCERDIRVNKVQQDC |
| Gly-Sub Con-P | GGGGHSKYQGCLRGIRVNKVQQGC |
| Naked Con-G | GEEELQENQELIREKSN |
| Naked Con-G E7A | GEEELQANQELIREKSN |
| S. (short) Naked | GEEELQENQELIR |
| Con-G 1-13 | |
| S. Naked Con-G | GEEELQANQELIR |
| E7A 1-13 | |
| Alanine-substituted | GAAALQANQALIRAKSN |
| Con-G | |
| Naked Con-Pr3 | GEPEVAKWAEGLREKAASN |
| Con-G-like APP | ADTDYADGSEDKVVE |
| (cAPP) | |
| Scrambled naked | NELQQESILERKENEGE |
| Con-G | |

or one or more amino acid sequences having at least 80% sequence homology thereto.

### Formula 252

In an embodiment, the NMDA receptor antagonist may be selected from one of the following peptides according to Formula 252. Such peptides are described in WO 2019/074858, published Apr. 18, 2019, corresponding to PCT/US2018/054889, filed Oct. 8, 2018, which are each incorporated by reference in their entirety herein. In the case of any conflict of terminology of Formula 252, these references incorporated by reference herein control.

In an embodiment, the NMDA receptor antagonist is a peptide comprising one or more amino acid sequences according to:

GSMCMPCFTTDHQMARRCDDCCGGRGRGRCYGPQCLCR
GSEVIRCSGSKQCYGPCKQQTGCTNSKCMNKVCKCYGCG
GSSEKDCIKHLQRCRENKDCCSKKCSRRGTNPEKRCR
GSSCAKPRENCNRMNILCCRGECVCPTFGDCFCYGD
GSGVPINVKCRGSRDCLDPCKKAGMRFGKCINSKCHCTP
GSVRIPVSCKHSGQCLKPCKDAGMRFGKCMNGKCDCTPK
GSDCVRFWGKCSQTSDCCPHLACKSKWPRNICVWDGSVG
GSMCMPCFTTRPDMAQQCRACCKGRGKCFGPQCLCGYD
GSGCFGYKCDYYKGCCSGYVCSPTWKWCVRPGPGR
GSQVQTNVKCQGGSCASVCRREIGVAAGKCINGKCVCYRN
GSGDCLPHLKRCKENNDCCSKKCKRRGANPEKRCR
GSNFKVEGACSKPCRKYCIDKGARNGKCINGRCHCYY
GSQKILSNRCNNSSECIPHCIRIFGTRAAKCINRKCYCYP
GSDRDŚCIDKSRCSKYGYYQECQDCCKKAGHNGGTCMFFKCKCA
GSAVCNLKRCQLSCRSLGLLGKCIGDKCECVKHG
GSQFCGTNGKPCVNGQCCGALRCVVTYHYADGVCLKMNP
GSRPTDIKCSASYQCFPVCKSRFGKINGRCVNGLCDCF
GSNCAGYMRECKEKLCCSGYVCSSRWKWCVLPAPWRR
GSQFTDVKCTGSKQCWPVCKQMFGKPNGKCMNGKCRCYS
GSQIDTNVKCSGSSKCVKICIDRYNTRGAKCINGRCTCYP
GSAEIIRCSGTRECYAPCQKLTGCLNAKCMNKACKCYGCV
GSSDYCSNDFCFFSCRRDRCARGDCENGKCVCKNCHLN
GSCIGEGVPCDENDPRCCFGLVCLKPTLHIGIWYKSYYCYKK
GSSCAKPGEMCMRIKCCDGQCGCNRGTGRCFCK
GSACLAEYQKCEGSTVPCCPGLSCSAGRFRKTKLCTK
GSVVIGQRCYRSPDCYSACKKLVGKATGKCTNGRCDC
GSACQFWSCNSSCISRGYRQGYCWGIQYKYCQCQ
GSRCPPCFTTNPNMEADCRKCCGGRGYCASYQCICPGG
GSVFINVKCRGSPECLPKCKEAIGKSAGKCMNGKCKCYP
GSQVSTNKKCSNTSQCYKTCEKVVGVAAGKCMNGKCICYP
GSECLEIFKACNPSNDQCCKSSKLVCSRKTRWCKYQIG
GSQDKCKKVYENYPVSKCQLANQCNYDCKLDKHARSGE
CFYDEKRNLQCICDYCEY
GSGVPINVRCRGSRDCLDPCRRAGMRFGRCINSRCHCTP
GGYSRCQLQGFNCVVRSYGLPTIPCCRGLTCRSYFPGSTYGRCQRY
GSMCMPCFTTDHQMARKCDDCCGGKGRGKCYGPQCLCR
GSAQEPVKGPVSTKPGSCPIILIRCAMLNPPNRCLKDTDCP
GIKKCCEGSCGMACFVPQ
GSMCMPCFTTDPNMAKKCRDCCGGNGKCFGPQCLCNR
GSGIGDPVTCLKSGAICHPVFCPRRYKQIGTCGLPGTKCCKKP
GSGVPTDVKCRGSPQCIQPCKDAGMRFGKCMNGKCHCTPK
GSRCPPCFTTNPNMEADCRKCCGGRGYCASYQCICPGG
MCMPCFTTDHQMARRCDDCCGGRGRGRCYGPQCLCR
EVIRCSGSKQCYGPCKQQTGCTNSKCMNKVCKCYGCG
SEKDCIKHLQRCRENKDCCSKKCSRRGTNPEKRCR
SCAKPRENCNRMNILCCRGECVCPTFGDCFCYGD
GVPINVKCRGSRDCLDPCKKAGMRFGKCINSKCHCTP
VRIPVSCKHSGQCLKPCKDAGMRFGKCMNGKCDCTPK
DCVRFWGKCSQTSDCCPHLACKSKWPRNICVWDGSVG
MCMPCFTTRPDMAQQCRACCKGRGKCFGPQCLCGYD
GCFGYKCDYYKGCCSGYVCSPTWKWCVRPGPGR
QVQTNVKCQGGSCASVCRREIGVAAGKCINGKCVCYRN
GDCLPHLKRCKENNDCCSKKCKRRGANPEKRCR
NFKVEGACSKPCRKYCIDKGARNGKCINGRCHCYY
QKILSNRCNNSSECIPHCIRIFGTRAAKCINRKCYCYP
DRDSCIDKSRCSKYGYYQECQDCCKKAGHNGGTCMFFKCKCA
AVCNLKRCQLSCRSLGLLGKCIGDKCECVKHG
QFCGTNGKPCVNGQCCGALRCVVTYHYADGVCLKMNP
RPTDIKCSASYQCFPVCKSRFGKTNGRCVNGLCDCF
NCAGYMRECKEKLCCSGYVCSSRWKWCVLPAPWRR
QFTDVKCTGSKQCWPVCKQMFGKPNGKCMNGKCRCYS
QIDTNVKCSGSSKCVKICIDRYNTRGAKCINGRCTCYP
AEURCSGTRECYAPCQKLTGCLNAKCMNKACKCYGCV
SDYCSNDFCFFSCRRDRCARGDCENGKCVCKNCHLN
CIGEGVPCDENDPRCCFGLVCLKPTLHGIWYKSYYCYKK
SCAKPGEMCMRIKCCDGQCGCNRGTGRCFCK
ACLAEYQKCEGSTVPCCPGLSCSAGRFRKTKLCTK
VVIGQRCYRSPDCYSACKKLVGKATGKCTNGRCDC
ACQFWSCNSSCISRGYRQGYCWGIQYKYCQCQ
RCPPCFTTNPNMEADCRKCCGGRGYCASYQCICPGG
VFINVKCRGSPECLPKCKEAIGKSAGKCMNGKCKCYP
QVSTNKKCSNTSQCYKTCEKVVGVAAGKCMNGKCICYP
ECLEIFKACNPSNDQCCKSSKLVCSRKTRWCKYQIG
QDKCKKVYENYPVSKCQLANQCNYDCKLDKHARSGECFY
DEKRNLQCICDYCEY
GVPINVRCRGSRDCLDPCRRAGMRFGRCINSRCHCTP
YSRCQLQGFNCVVRSYGLPTIPCCRGLTCRSYFPGSTYGRCQRY
MCMPCFTTDHQMARKCDDCCGGKGRGKCYGPQCLCR
AQEPVKGPVSTKPGSCPIILIRCAMLNPPNRCLKDTDCPGI
KKCCEGSCGMACFVPQ
MCMPCFTTDPNMAKKCRDCCGGNGKCFGPQCLCNR
GIGDPVTCLKSGAICHPVFCPRRYKQIGTCGLPGTKCCKKP
GVPTDVKCRGSPQCIQPCKDAGMRFGKCMNGKCHCTPK
RCPPCFTTNPNMEADCRKCCGGRGYCASYQCICPGG

or one or more amino acid sequences having at least 80% homology thereto.

### Formula 253

In an embodiment, the NMDA receptor antagonist may be selected from one of the following peptides according to Formula 253. Such peptides are described in CN 109553685, filed Sep. 25, 2017 which are each incorporated by reference in their entirety herein. In the case of any conflict of terminology of Formula 253, these references incorporated by reference herein control.

In an embodiment, the NMDA receptor antagonist is a polypeptide comprising one or more of an NR^{2B} (NMDA receptor subunit NR^{2B}) sequence, a CTM (chaperone-mediated autophagy targeting motif) sequence, and a cell-penetrating peptide (such as a TAT cell-penetrating peptide or others).

In an embodiment, the NMDA receptor antagonist comprises one or more of an TAT cell-penetrating peptide sequence having a polypeptide sequence having at least 80% homology to YGRKKRRQRRR; an NR^{2B} peptide sequence having a polypeptide sequence having at least 80% homology to RRPPRSPDHKRYFRDKE; and a CTM sequence having at least 80% homology to KFERQKILDQRFFE.

In further embodiments, the NMDA receptor antagonist comprises the sequence YGRKKRRQRRRRRPPRSPDHKRYFRDKEKFERQKILDQRFFE, or a sequence having at least 80% homology thereto.

### Formula 254

In an embodiment, the NMDA expression may be selectively reduced by administration or dosing with a peptide according to Formula 254. Such peptides are described in WO2019/036511, published Feb. 21, 2019 and corresponding to PCT/US2018/046770, filed Aug. 14, 2018, which are each incorporated by reference in their entirety herein. In the case of any conflict of terminology of Formula 254, these references incorporated by reference herein control.

In an embodiment, the peptide comprises the polypeptide sequence VSGLNPSLWSIFGLQFILLWLVSGSRHYLW or YGRKKRRQRRRVSGLNPSLWSIFGLQFILLWLVSGSRHYLW, or a sequence having at least 80% sequence homology with either of these sequences. In further embodiments, the peptide may be a polypeptide having portions (for example, three or more consecutive amino acids) of either of these sequences and having an overall length of 10-50 residues.

### Formula 255

In an embodiment, the NMDA receptor antagonist may be selected from one of the following peptides according to Formula 255. Such peptides are described in WO 2010/004003, published Jan. 14, 2010, corresponding to PCT/EP2009/058752, filed Jul. 9, 2009, and U.S. Pat. No. 9,241,967, issued Jan. 26, 2016, which are each incorporated by reference in their entirety herein. In the case of any conflict of terminology of Formula 255, these references incorporated by reference herein control.

A compound comprising a first peptide or peptide analogue linked to a second peptide or peptide analogue by a linker, optionally a PEG linker, wherein said linker is associated with a N-terminus of said first and second peptide or peptide analogue, and wherein said first and said second peptide or peptide analogue comprise at least four amide-bonded residues having a sequence Xaa1-Thr-Xaa3-Val or Xaa1-Ser-Xaa3-Val, wherein: a. Xaa1 is selected from among E, Q, and A, or an analogue thereof, and b. Xaa3 is selected from among A, Q, D, N, N-Me-A, N-Me-Q, N-Me-D, and N-Me-N or an analogue thereof.

In further embodiments, the compound is selected from:

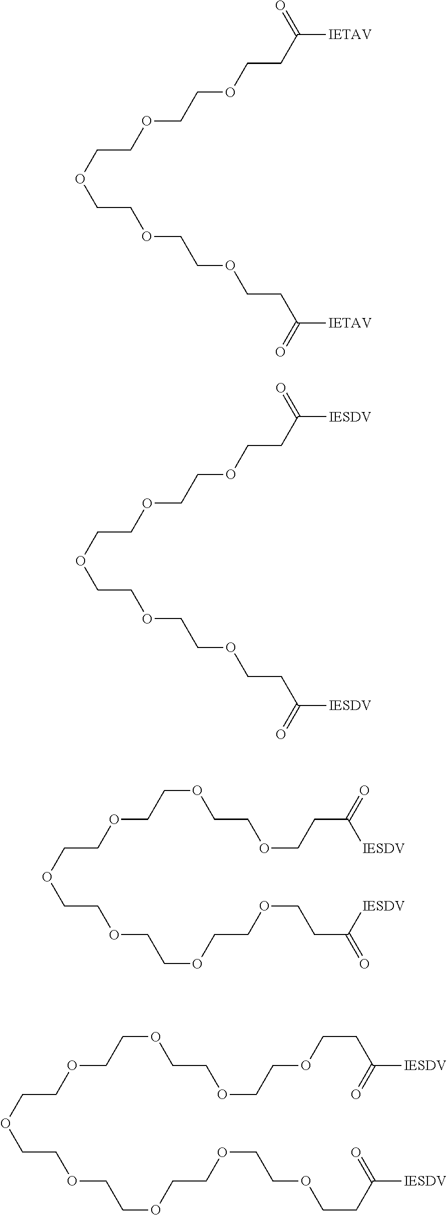

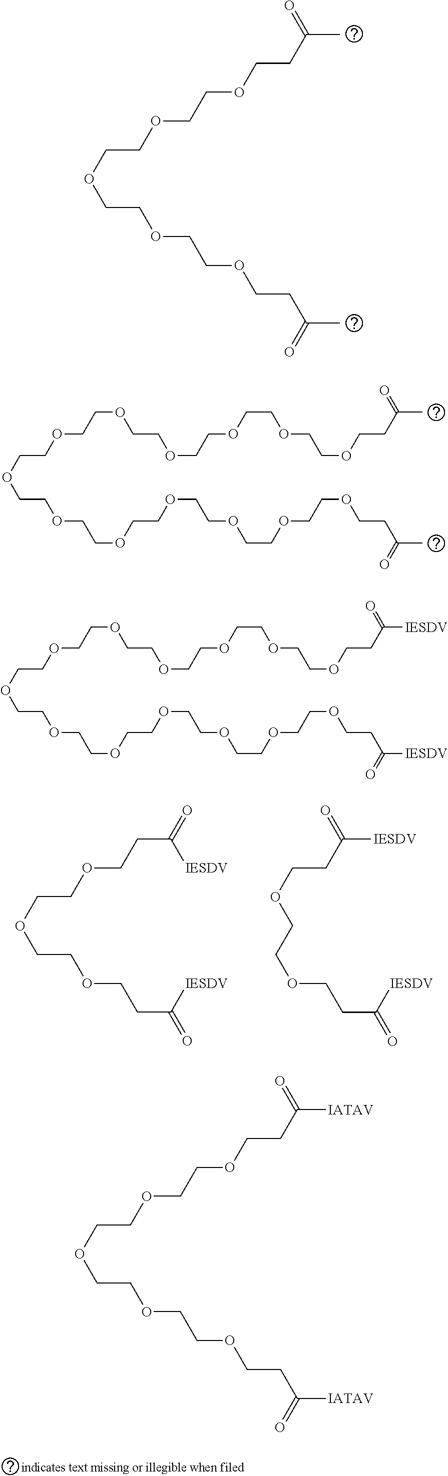

### Formula 256

In an embodiment, the NMDA receptor antagonist may be selected from one of the following peptides according to Formula 256. Such peptides are described in WO 2014/096402, published Jun. 26, 2014 corresponding to PCT/EP2013/077780, filed Dec. 20, 2013 and US 2015/0337276, published Nov. 26, 2015, which are each incorporated by reference in their entirety herein. In the case of any conflict of terminology of Formula 256, these references incorporated by reference herein control.

In an embodiment, the NMDA receptor antagonist is an enzyme, optionally a metalloenzyme comprising at least one divalent cation. In an embodiment, the divalent cation is Zn2+, Mg2+, Ni2+, Cd2+, Mn2+, Co2+, Fe2+, and Ag2+, preferably is Zn2+.

In further embodiments, the enzyme is a phosphotriesterase or phosphotriesterase derivative.

In further embodiments, the enzyme comprises a sequence, or functional portion thereof, having a sequence according to:

MQTRRVVLKSAAAAGTLLGGLAGCASVAGSIGTGDRINTVRGPIT
ISEAGFTLTHEHICGSSAGFLRAWPEFFGSRKALAEKAVRGLRRA
RAAGVRTIVDVSTFDIGRDVSLLAEVSRAADVHIVAATGLWFDPP
LSMRLRSVEELTQFFLREIQYGIEDTGIRAGIIKVATTGKATPFQ
ELVLKAAARASLATGVPVTTHTAASQRDGEQQAAIFESEGLSPSR
VCIGHSDDTDDLSYLTALAARGYLIGLDHIPHSAIGLEDNASASA
LLGIRSWQTRALLIKALIDQGYMKQILVSNDWLFGFSSYVTNIMD
VMDRVNPDGMAFIPLRVIPFLREKGVPQETLAGITVTNPARFLSP
TLRAS;
or
SIGTGDRINTVRGPITISEAGFTLTHEHICGSSAGFLRAWPEFFG
SRKALAEKAVRGLRRARAAGVRTIVDVSTFDIGRDVSLLAEVSRA
ADVHIVAATGLWFDPPLSMRLRSVEELTQFFLREIQYGIEDTGIR
AGIIKVATTGKATPFQELVLKAAARASLATGVPVTTHTAASQRDG
EQQAAIFESEGLSPSRVCIGHSDDTDDLSYLTALAARGYLIGLDH
IPHSAIGLEDNASASALLGIRSWQTRALLIKALIDQGYMKQILVS
NDWLFGFSSYVTNIMDVMDRVNPDGMAFIPLRVIPFLREKGVPQE
TLAGITVTNPARFLSPTLRAS;
or
MSIGTGDRINTVRGPITISEAGFTLTHEHICGSSAGFLRAWPEFF
GSRKALAEKAVRGLRRARAAGVRTIVDVSTFDIGRDVSLLAEVSR
AADVHIVAATGLWFDPPLSMRLRSVEELTQFFLREIQYGIEDTGI
RAGIIKVATTGKATPFQELVLKAAARASLATGVPVTTHTAASQRD
GEQQAAIFESEGLSPSRVCIGHSDDTDDLSYLTALAARGYLIGLD
HIPHSAIGLEDNASASALLGIRSWQTRALLIKALIDQGYMKQILV
SNDWLFGFSSYVTNIMDVMDRVNPDGMAFIPLRVIPFLREKGVPQ
ETLAGITVTNPARFLSPTLRAS;

or a sequence comprising a portion having at least 80% sequence homology to any of these sequences.

### Formula 257

In an embodiment, the NMDA receptor antagonist may be selected from one of the following peptides according to Formula 257. Such peptides are described in WO 2013/185572, published Dec. 19, 2013 and corresponding to PCT/CN2013/076967, filed Jun. 8, 2013, and U.S. Pat. No. 9,718,864, issued Aug. 1, 2017. which are each incorporated by reference in their entirety herein. In the case of any conflict of terminology of Formula 257, these references incorporated by reference herein control.

In an embodiment, the NMDA receptor antagonist is a polypeptide comprising one or more sequences according to:

MKLTCVLIITVLFLTACQLTTAVTYSRGEHKHRALMST
GTNYRLPKTCRSSGRYCRSPYDCRRRYCRRITDACV;
or
MKLTCVVIITVLFLTACQLTTAVTYSRGEHKHRALMST
GTNYRLPKTCRSSGRYCRSPYDCRRRYCRRITDACV;
or
TCRSSGRYCRSPYDCRRRYCRRITDACV;
or
MKLTCVLIITVLFLTACQLTTAVTYSRGEHKHRALMS
TGTNYRLPKTCRSSGRYCRSPYDRRRRYCRRITDAC
V;
or
MKLTCVVIITVLFLTACQLTTAVTYSRGEHKHRALM
STGTNYRLPKTCRSSGRYCRSPYDRRRRYCRRITDA
CV;
or
TCRSSGRYCRSPYDRRRRYCRRITDACV,

or a sequence comprising a portion having at least 80% homology with any one or more of these sequences.

### Formula 258

In an embodiment, the NMDA receptor antagonist (blocker) may be selected from one of the following peptides according to Formula 258. Such peptides are described in WO2008/014917, published Feb. 7, 2008, corresponding to PCT/EP2007/006619, filed Jul. 25, 2007, which are each incorporated by reference in their entirety herein. In the case of any conflict of terminology of Formula 258, these references incorporated by reference herein control.

In an embodiment, the NMDA receptor antagonist is a polypeptide comprising entirely D-enantiomeric amino acids.

In an embodiment, the NMDA receptor antagonist is a fusion peptide comprising at least a component (I), wherein component (I) comprises a transporter peptide, and a component (II), selected from a peptide inhibiting interaction of neuronal N-methyl-D-aspartate receptor (NMDAR) with NMDAR interacting proteins, wherein component (II) entirely consists of D-enantiomeric amino acids.

In an embodiment, component (I) is selected from cell penetrating peptides including: (a)protein transduction domains (PTD) and protein derived CPPs, including sequences derived from Antennapedia, pAntp (43-58), comprising the sequences RQI KIVVFQN RRMKWKK or RQIKIWFQNRRMKWKK-amide, including sequences derived from human immunodeficiency virus 1 (HIV-1), TAT, region 37 to 72, region 37 to 60, region 48 to 60 or region 49 to 57 from TAT, sequences GRKKRRQRRR, YGRKKRRQRRR, CGRKKRRQRRRPPQC or CGRKKRRQRRRPPQCC, including hCT(9-32) having the sequence LGTYTQDFNKFHTFPQTAIGVGAP-NH2, pVEC comprising the sequence LLIILRRRIRKQAHAHSK-NH2, plSL comprising the sequence RVIRVWFQNKRCKDKK-NH2, mouse PRP (1-28) comprising the sequence MANGLYWLLALFVTMWTDVGLCKKRPKP-NH2 and homologs thereto including human homologs, Ems (194-220) comprising the sequence RQGAARVTSWLGLQLRIAGKRLEGRSK-NH2, Restricocin L3 (60-73) comprising the sequence KLIKGRTPIKFGK-NH2, or
(b) model peptides including VT5 comprising the sequence DPKGDPKGVTVTVTVTVTGKGDPKPD-NH2, MAP comprising the sequence KLALKLALKALKAALKLA-NH2, arginine stretches including RRRRRRR, i.e. (Arg)7, or RRRRRRR-NH2, i.e. (Arg)7-NH2, or RRRRRRR-C, i.e. (Arg)7-C, or RRRRRRRR, i.e. (Arg)8, or RRRRRRRR-NH2, i.e. (Arg)8-NH₂, RRRRRRRRR, i.e. (Arg)9, or RRRRRRRRR-NH2, i.e. (Arg)9-NH2, or
(c) designed CPPs including MPG comprising the sequence GALFLGFLGAAGSTMGAWSQPKSKRKV or GALFLGFLGAAGSTMGAWSQPKSKRKV-cysteamide;
Transportan comprising the sequence GWTLNSAGYLLGKINLKALAALAKKIL-NH2 (SEQ, Transportan 10 comprising the sequence AGYLLGKINLKALAALAKKIL-NH2, Pep-1 comprising the sequence KETWWETWWTEWSQPKKKRKV or KETWWETWWTEWSQPKKKRKV-cysteamide, or may be selected from the KALA peptide comprising the sequence WEAKLAKALAKALAKHLAKALAKALKACEA or from Bulforin 2 comprising the sequence TRSSRAGLQFPVGRVHRLLRK, or an retro-inverso isomer of any of these sequences composed of D amino acids, or any sequences having at least 80% homology with any of these sequences.

In an embodiment, component (II) comprises the polypeptide sequence vdseisslk, or a sequence having at least 80% homology thereto. In an embodiment, the component (II) sequence comprises a length of 5 to 40 amino acids.

### Formula 259

In an embodiment, the NMDA receptor antagonist may be selected from one of the following peptides according to Formula 259 Such peptides are described in U.S. Pat. No. 5,854,217, issued Dec. 29, 1998, and U.S. Pat. No. 6,284,731, issued Sep. 4, 2001, which are each incorporated by reference in their entirety herein. In the case of any conflict of terminology of Formula 259, these references incorporated by reference herein control.

In an embodiment, the NMDA receptor antagonist comprises one or more sequences according to:

Asn Gly Glu Gla Gla Leu Gln Gla Asn Gln Gla Leu Ile Arg Gla Lys Ser Asn-NH2;
Phe Gly Glu Gla Gla Leu Gln Gla Asn Gln Gla Leu Ile Arg Gla Lys Ser Asn-NH2;
t-BuTyr Gly Glu Gla Gla Leu Gln Gla Asn Gln Gla Leu Ile Arg Gla Lys Ser Asn-NH2;
Ser Gly Glu Gla Gla Leu Gln Gla Asn Gln Gla Leu Ile Arg Gla Lys Ser Asn-NH2;
Gly Glu Ser Gla Leu Gln Gla Asn Gln Gla Leu Ile Arg Gla Lys Ser Asn-NH2,
wherein the serine at position 3 is phosphorylated;
Gly Glu Ala Gla Leu Gln Gla Asn Gln Gla Leu Ile Arg Gla Lys Ser Asn-NH2;
Gly Glu Gla Gla Leu Gln Tyr Asn Gln Gla Leu Ile Arg Gla Lys Ser Asn-NH2;
Gly Glu Gla Gla Leu Gln Ala Asn Gln Gla Leu Ile Arg Gla Lys Ser Asn-NH2;
Gly Glu Gla Gla Leu Gln Ser Asn Gln Gla Leu Ile Arg Gla Lys Ser Asn-NH2;
Gly Glu Gla Gla Leu Gln Ser Asn Gln Gla Leu Ile Arg Gla Lys Ser Asn-NH2
wherein the first serine is phosphorylated;
Gly Glu Gla Gla Leu Gln Gla Asn Gln Ala Leu Ile Arg Gla Lys Ser Asn-NH2;
Gly Glu Gla Gla Leu Gln Gla Asn Gln Ser Leu Ile Arg Gla Lys Ser Asn-NH2;
Gly Glu Gla Gla Leu Gln Gla Asn Gln Ser Leu Ile Arg Gla Lys Ser Asn-NH2,
wherein the first serine is phosphorylated;
Gly Glu Gla Gla Leu Gln Gla Asn Gln Gla Leu Ile Arg Ala Lys Ser Asn-NH2;
Gly Glu Gla Gla Leu Gln Gla Asn Gln Gla Leu Ile Arg Ser Lys Ser Asn-NH2;
Gly Glu Gla Gla Leu Gln Gla Asn Gln Gla Leu Ile Arg Ser Lys Ser Asn-NH2,
wherein the first serine is phosphorylated;
Gly Glu Gla Gla Leu Gln Ala Asn Gln Ala Leu Ile Arg Ala Lys Ser Asn-NH2;
and
Gla Lys Ser Asn-NH2;
Tyr Gly Glu Glu Glu Leu Gln Glu Asn Gln Glu Leu Ile Arg Glu Lys Ser Asn-NH2;
and
Glu Glu Glu Leu Gln Glu Asn Gln Glu Leu Ile Arg Glu Lys Ser Asn-NH2.

In further embodiments, the NMDA receptor antagonist has the structure:

A1-A2-A3-A4-A5-A6-(A7)x c-[A8-(A9-A10)x d-NH2]n

wherein
A1 is glycine, alanine, valine, leucine or isoleucine;
A2 is glutamic acid, aspartic acid, γ-carboxyglutamate (Gla), 3-carboxyaspartic acid, D-glutamic acid, phosphoserine or phosphothreonine;
A3 is glutamic acid, aspartic acid, γ-carboxyglutamate (Gla), 3-carboxyaspartic acid, D-glutamic acid, phosphoserine or phosphothreonine;
A4 is Gla;
A5 is glycine, alanine, valinc, leucine or isoleucine;
A6 is a peptide of 7-9 amino acids;
A7 is an amino acid selected from the group consisting of natural, modified or non-natural amino acids;
A8 is lysine or arginine;
A9 is an amino acid selected from the group consisting of natural, modified or non-natural amino acids;
A10 is an amino acid selected from the group consisting of natural, modified or non-natural amino acids;
xc and xd are independently 0 or 1; and
   n is 0 or 1.

### Formula 260

In yet further embodiments, the NMDA receptor antagonist is a histogranin or analog thereof, as described in CA 2097533, published Dec. 4, 1993.

### Pharmaceutical Compositions

In an embodiment, provided is a pharmaceutical composition comprising a metal channel activator and a NMDA receptor antagonist.

In another embodiment, the metal channel activator in said pharmaceutical composition is a potassium channel activator.

In another embodiment, the potassium channel activator in said pharmaceutical composition is a Kv7 channel activator.

In another embodiment, the Kv7 channel activator in said pharmaceutical composition is selected from the group consisting of a Kv7.1 channel activator, a Kv7.2 channel activator, a Kv7.3 channel activator, a Kv7.4 channel activator, a Kv7.5 channel activator, or any combination thereof.

In another embodiment, the Kv7 channel activator in said pharmaceutical composition is a Kv7.2/7.3 channel activator.

In another embodiment, the metal channel activator in said pharmaceutical composition is selected from one or more of the disclosed compounds according to any one or more of formulas 1 to 171 and the compounds disclosed in the “further embodiments” under the “METAL CHANNEL ACTIVATORS” subheading.

In another embodiment, the NMDA receptor antagonist in said pharmaceutical composition is selected from one or more of the disclosed compounds according to any one or more of formulas 200 to 260.

In another embodiment, the NMDA receptor antagonist in said pharmaceutical composition is selected from one or more of the disclosed compounds according to any one or more of formulas 200 to 260, and the metal channel activator in said pharmaceutical composition is selected from one or more of the disclosed compounds according to any one or more of formulas 1 to 171 and the compounds disclosed in the “further embodiments” under the “METAL CHANNEL ACTIVATORS” subheading.

In another embodiment, the NMDA receptor antagonist or a pharmaceutically acceptable salt thereof in said pharmaceutical composition is in a form of a prodrug.

### Methods of Treating Diseases

The combination therapies (comprising at least one of any metal channel activator described herein and at least one NMDA receptor antagonist described herein) disclosed herein are useful for treating various depressive disorders. The depressive disorder include major depressive disorder (MDD), disruptive mood dysregulation disorder, persistent depressive disorder, bipolar spectrum disorder, postpartum depression, premenstrual dysphoric disorder (PMDD), seasonal affective disorder (SAD), atypical depression, treatment-resistant depression (TRD), depression associated with agitation or anxiety, adjustment disorder with depressed mood, prolonged depressive reaction, or a combination thereof.

The combination therapies disclosed herein are useful for treating various neurological and neurodegenerative disorders. These disorders include amyotrophic lateral sclerosis, bipolar disorder, treatment resistant and major depression, general anxiety disorder, panic disorder, social anxiety, mood disorders, cognitive disorders, dementia, agitation, apathy, psychoses, post-traumatic stress disorders, irritability, disinhibition, learning disorders, memory loss, personality disorders, bipolar disorders, Rett syndrome, eating disorders, conduct disorder, neurodegenerative disorders, pain disorders, supranuclear palsy, frontotemporal dementia, frontotemporal lobar degeneration, delirium, Alzheimer's disease, mild cognitive impairment, mild cognitive impairment due to Alzheimer's disease, drug addiction, tinnitus, mental retardation, obsessive-compulsive disorder, spinal muscular atrophy, radiation therapy, multiple sclerosis, chronic cerebellar ataxia, cervical spondylotic myelopathy, spinal cord injury, hereditary cerebellar ataxia, Tourette syndrome, autism spectrum disorder, schizophrenia, fragile X syndrome, Parkinson's Disease, Huntington's disease, or any combination thereof.

The combination therapies disclosed herein are useful for treating various pain disorders. The pain disorders include acute pain, chronic pain, neuropathic pain, nociceptive pain, radicular pain. The pain disorders may also include migraine, inflammatory pain, persistent pain, cancer pain, and postoperative pain.

The metal channel activator and the NMDA receptor antagonist may be given orally, sublingually, subcutaneously or in any other means of delivery. The agents may be delivered simultaneously or sequentially. If the agents are delivered sequentially, either agent may be dosed first, and the separation of time may include finishing the dosing of one agent completely before commencing the dosage of the other or they may be intermingled in time.

In an embodiment, provided is a kit for treating a patient afflicted with at least one of the aforementioned disorders, said kit including:
(a) a metal channel activator; and
(b) instructions for administering said metal channel activator in combination with an NMDA receptor antagonist by one of the aforementioned methods.

In another embodiment, provided is a kit for treating a patient afflicted with at least one of the aforementioned disorders, said kit including:
(a) an NMDA receptor antagonist; and
(b) instructions for administering said NMDA receptor antagonist with a metal channel activator by one of the aforementioned methods.

As used herein, the term “metal channel activator” is construed to include both metal channel activator and pharmaceutically acceptable salt thereof.

As used herein, the term “NMDA receptor antagonist” is construed to include both NMDA receptor antagonist and pharmaceutically acceptable salt thereof.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

### Pharmaceutical Compositions and Methods of Treatment

Compounds are generally given as pharmaceutical compositions comprised of a therapeutically effective amount of one or more of a compound of any one of Formulas 1-180, Formulas 200-260, those listed in “Further Embodiments” under the “METAL CHANNEL ACTIVATORS” subsection, or pharmaceutically acceptable salt(s) thereof, and a pharmaceutically acceptable carrier, further optionally containing conventional excipients. A therapeutically effective amount is the amount needed to provide a meaningful patient benefit as determined by practitioners in that art. Pharmaceutically acceptable carriers are those having acceptable safety profiles which are conventionally known. Compositions encompass common solid and liquid forms including but not limited to capsules, tablets, lozenges, liquid suspensions, syrups, elixirs, and solutions. Solid compositions may by formulated to timed or sustained release. Compositions are made using common formulation techniques, such as the aforementioned solid and liquid forms, conventional excipients, such as binding and wetting agents, and vehicles, such as water and alcohols.

The NMDA receptor antagonists of the present invention may be given orally, sublingually, intranasally, buccally, subcutaneously or in any other suitable means of delivery in table 1. The NMDA receptor antagonists may be in the form of a prodrug, which releases the agent in the body, a sustained release vehicle, a delayed release vehicle, or any other suitable delivery form. The NMDA receptor antagonists and the metal channel activator may be delivered simultaneously or sequentially. If the agents are delivered sequentially, either agent may be dosed first, and the separation of time may include finishing the dosing of one agent completely before commencing the dosage of the other or they may be intermingled in time. Typically, the NMDA receptor antagonist is administered at a time proximate to the administration of the metal channel activator, e.g., within 1 week, 1 day, 1 hour, 1 minute before or after the metal channel activator or simultaneously with the metal channel activator.

The metal channel activators of the present invention may be given orally, sublingually, intranasally, buccally, subcutaneously or in any other suitable means of delivery in table 1. Metal channel activators may be given in the form of a prodrug, which will release the active compound in the body, a delayed release formulation, which will release the active compound after a time delay, a sustained release formulation, which will release the active compound slowly over time, or any other suitable formulation to deliver the active ingredient. The metal channel activator may be delivered simultaneously or sequentially with the NMDA receptor antagonist. If the agents are delivered sequentially, either agent may be dosed first, and the separation of time may include finishing the dosing of one agent completely before commencing the dosage of the other or they may be intermingled in time. Typically, the metal channel activator will be administered at a time nearing the administration of the NMDA receptor antagonist e.g., within 1 week, 1 day, 1 hour, 1 minute before or after the NMDA receptor antagonist or simultaneously with the metal channel activator.

The dose of the NMDA receptor antagonist and metal channel activator for use together may depend on the subject to be treated inclusive of the age, sex, weight, and general health condition thereof. In this regard, precise amounts of the agent(s) for administration will depend on the judgment of the practitioner. In determining the effective amount of the NMDA receptor antagonist and metal channel activator to be administered in the treatment or reducing of the conditions associated with the symptoms and disorders, the physician may evaluate clinical factors including symptoms severity or progression of the disorder. In some conditions, a rapid absorption of the NMDA receptor antagonist or metal channel activator may be desirable. The effective amount of the treatment will vary depending on the subject and disease state being treated, the severity of the affliction and the manner of administration, and may be determined routinely by one of ordinary skill in the art.

Some of the NMDA receptor antagonists and metal channel activators can be administered sublingually. The sublingual formulation may be administered in an effective amount to a subject in need thereof. The subject may be an animal or human. When the NMDA receptor antagonist or metal channel activator is prepared as a sublingual formulation, the sublingually administered chemical agent or the drug can diffuse into capillaries through mucous membrane under the tongue, and then enter venous circulation of the subject. As such, sublingual administration may have advantages over oral administration as allowing for direct or faster entry to venous circulation, without risks of degradation in gastrointestinal tract, alteration by drug metabolism in liver and the like.

A sublingual formulation useful in the present invention comprises an effective amount of an NMDA receptor antagonist, metal channel activator, or pharmaceutically acceptable salts, solvates, anomers, enantiomers, hydrates, or prodrugs thereof. The formulation provides sufficient solubility for a NMDA receptor antagonist and/or metal channel activator to be incorporated into the sublingual formulation at relatively large doses and sublingually delivered. The formulation is preferably a modified oral disintegrating formulation of a NMDA receptor antagonist and/or metal channel activator. The excipients, including mannitol and gelatin, are blended, solubilized with water and deaerated before being mixed with the active pharmaceutical ingredient (or “API”), a NMDA receptor antagonist and/or a metal channel activator, which have been milled separately. Particle size of the API (D50) may be less than about 2 microns. The mixture is lyophilized by flash freezing and then freeze-dried. The formulation has good oral palatability. The effective amount of a NMDA receptor antagonist and/or metal channel activator for the sublingual formulation useful in the present invention to achieve a lower therapeutic dose may be less than that of orally administered agent. Moreover, effective dose of the sublingual formulation of the NMDA receptor antagonist and/or metal channel activator may be about 1 to 95% of that of the orally administered agent. Sublingual formulations of the metal channel activator and/or NMDA receptor antagonist may also have improved properties.

In one aspect of the invention, the NMDA receptor antagonist and/or metal channel activator is provided in a sublingual formulation in a form of an orally dissolving or disintegrating tablet (ODT). The ODT as used herein may be prepared by mixing the NMDA receptor antagonist and/or the metal channel activator with water-soluble diluents and compressed in a tablet. A suspension comprising the active product may be prepared with appropriate excipients and the suspension may be dispensed into blister packs and freeze-dried. An exemplary freeze-dried preparation platform that could be used for the ODT is the ZYDIS® (Catalent, Somerset, NJ, USA) formulation. In particular, the excipients, including water, are blended and the NMDA receptor antagonist and/or metal channel activator are separately milled to size and mixed with the excipients. The suspension then undergoes lyophilization by flash freezing and freeze drying. Other methods of preparing ODTs may be used without limitation, and detailed description of general methods thereof have been disclosed, for example, in U.S. Pat. Nos. 5,631,023; 5,837,287; 6,149,938; 6,212,791; 6,284,270; 6,316,029; 6,465,010; 6,471,992; 6,471,992; 6,509,040; 6,814,978; 6,908,626; 6,908,626; 6,982,251; 7,282,217; 7,425,341; 7,939,105; 7,993,674; 8,048,449; 8,127,516; 8,158,152; 8,221,480; 8,256,233; and 8,313,768, each of which is incorporated herein by reference in its entirety.

The clinical or therapeutic effect of the sublingually formulated NMDA receptor antagonist may have an improved pharmacokinetic profile for the pharmaceutical agent as measured by standard testing parameters. When the NMDA receptor antagonist is administered sublingually, the Tₘₐₓ, Cₘₐₓ and AUC of the drug may be improved compared to the same dose of the orally administered version of the same compound. For example, the sublingual formulation of the NMDA receptor antagonist may have a greater Cₘₐₓ than the orally administered NMDA receptor antagonist to provide a therapeutically beneficial effect. The sublingual formulation of the NMDA receptor antagonist may have an earlier or lesser Tₘₐₓ than the orally administered NMDA receptor antagonist to provide a therapeutically beneficial effect and in some instances, a more rapid therapeutic effect. Alternatively, the sublingual formulation of the NMDA receptor antagonist may have a greater AUC per milligram of the agent than the orally administered NMDA receptor antagonist. In addition, as the NMDA receptor antagonist may make the metal channel activator more effective, lesser amounts of the metal channel activator may be needed to achieve the same results, lessening the inherent side effects.

The clinical or therapeutic effect of the sublingually formulated metal channel activator may have an improved pharmacokinetic profile for the pharmaceutical agent as measured by standard testing parameters. When the metal channel activator is administered sublingually, the Tₘₐₓ, Cₘₐₓ and AUC of the drug may be improved compared to the same dose of the orally administered version of the same compound. For example, the sublingual formulation of the metal channel activator may have a greater Cₘₐₓ than the orally administered metal channel activator to provide a therapeutically beneficial effect. The sublingual formulation of the metal modulator may have an earlier or lesser Tₘₐₓ than the orally administered metal channel activator to provide a therapeutically beneficial effect and in some instances, a more rapid therapeutic effect. Alternatively, the sublingual formulation of the metal channel activator may have a greater AUC per milligram of the agent than the orally administered metal channel activator. In addition, as the metal channel activator may make the NMDA receptor antagonist more effective, lesser amounts of the NMDA receptor antagonist may be needed to achieve the same results, lessening the inherent side effects.

The disclosure and methods encompass all conventional modes of administration including oral, parenteral, intranasal, sublingual, topical, and transdermal methods. Among other routes of administration, the standard routes of administration described by the FDA are contemplated herein as shown in Table 1 below (FDA Routes of Administration; retrieved from www.fda.gov; content current as of Nov. 1, 2022).

TABLE 1
FDA Routes of Administration

| | | | | NCI |
|---|---|---|---|---|
| | | SHORT | FDA | CONCEPT |
| NAME | DEFINITION | NAME | CODE | ID |
| AURICULAR (OTIC) | Administration to or by way | OTIC | 013 | C38192 |
| | of the ear. | | | |
| BUCCAL | Administration directed | BUCCAL | 030 | C38193 |
| | toward the cheek, generally | | | |
| | from within the mouth. | | | |
| CONJUNCTIVAL | Administration to the | CONJUNC | 068 | C38194 |
| | conjunctiva, the delicate | | | |
| | membrane that lines the | | | |
| | eyelids and covers the | | | |
| | exposed surface of the eyeball. | | | |
| CUTANEOUS | Administration to the skin. | CUTAN | 130 | C38675 |
| DENTAL | Administration to a tooth or | DENTAL | 038 | C38197 |
| | teeth. | | | |
| ELECTRO-OSMOSIS | Administration of through the | EL-OSMOS | 357 | C38633 |
| | diffusion of substance through | | | |
| | a membrane in an electric | | | |
| | field. | | | |
| ENDOCERVICAL | Administration within the | E-CER VIC | 131 | C38205 |
| | canal of the cervix | | | |
| | uteri. Synonymous with the | | | |
| | term intracervical. | | | |
| ENDOSINUSIAL | Administration within the | E-SINUS | 133 | C38206 |
| | nasal sinuses of the head. | | | |
| ENDOTRACHEAL | Administration directly into | E-TRACHE | 401 | C38208 |
| | the trachea. | | | |
| ENTERAL | Administration directly into | ENTER | 313 | C38209 |
| | the intestines. | | | |
| EPIDURAL | Administration upon or over | EPIDUR | 009 | C38210 |
| | the dura mater. | | | |
| EXTRA-AMNIOTIC | Administration to the outside | X-AMNI | 402 | C38211 |
| | of the membrane enveloping | | | |
| | the fetus | | | |
| EXTRACORPOREAL | Administration outside of the | X-CORPOR | 057 | C38212 |
| | body. | | | |
| HEMODIALYSIS | Administration through | HEMO | 140 | C38200 |
| | hemodialysate fluid. | | | |
| INFILTRATION | Administration that results in | INFIL | 361 | C38215 |
| | substances passing into tissue | | | |
| | spaces or into cells. | | | |
| INTERSTITIAL | Administration to or in the | INTERSTIT | 088 | C38219 |
| | interstices of a tissue. | | | |
| INTRA-ABDOMINAL | Administration within the | I-ABDOM | 056 | C38220 |
| | abdomen. | | | |
| INTRA-AMNIOTIC | Administration within the | I-AMNI | 060 | C38221 |
| | amnion. | | | |
| INTRA-ARTERIAL | Administration within an | I-ARTER | 037 | C38222 |
| | artery or arteries. | | | |
| INTRA-ARTICULAR | Administration within a joint. | I-ARTIC | 007 | C38223 |
| INTRABILIARY | Administration within the bile, | I-BILI | 362 | C38224 |
| | bile ducts or gallbladder. | | | |
| INTRABRONCHIAL | Administration within a | I-BRONCHI | 067 | C38225 |
| | bronchus. | | | |
| INTRABURSAL | Administration within a bursa. | I-BURSAL | 025 | C38226 |
| INTRACARDIAC | Administration with the heart. | I-CARDI | 027 | C38227 |
| INTRACARTILAGINOUS | Administration within a | I-CARTIL | 363 | C38228 |
| | cartilage; endochondral. | | | |
| INTRACAUDAL | Administration within the | I-CAUDAL | 413 | C38229 |
| | cauda equina. | | | |
| INTRACAVERNOUS | Administration within a | I-CAVERN | 132 | C38230 |
| | pathologic cavity, such as | | | |
| | occurs in the lung in | | | |
| | tuberculosis. | | | |
| INTRACAVITARY | Administration within a non- | I-CAVIT | 023 | C38231 |
| | pathologic cavity, such as that | | | |
| | of the cervix, uterus, or penis, | | | |
| | or such as that which is | | | |
| | formed as the result of a | | | |
| | wound. | | | |
| INTRACEREBRAL | Administration within the | I-CERE | 404 | C38232 |
| | cerebrum. | | | |
| INTRACISTERNAL | Administration within the | I-CISTERN | 405 | C38233 |
| | cisterna magna | | | |
| | cerebellomedularis. | | | |
| INTRACORNEAL | Administration within the | I-CORNE | 406 | C38234 |
| | cornea (the transparent | | | |
| | structure forming the anterior | | | |
| | part of the fibrous tunic of the | | | |
| | eye). | | | |
| INTRACORONAL, | Administration of a drug | I-CORONAL | 117 | C38217 |
| DENTAL | within a portion of a tooth | | | |
| | which is covered by enamel | | | |
| | and which is separated from | | | |
| | the roots by a slightly | | | |
| | constricted region known as | | | |
| | the neck. | | | |
| INTRACORONARY | Administration within the | I- | 119 | C38218 |
| | coronary arteries. | CORONARY | | |
| INTRACORPORUS | Administration within the | I-CORPOR | 403 | C38235 |
| CAVERNOSUM | dilatable spaces of the corpora | | | |
| | cavernosa of the penis. | | | |
| INTRADERMAL | Administration within the | I-DERMAL | 008 | C38238 |
| | dermis. | | | |
| INTRADISCAL | Administration within a disc. | I-DISCAL | 121 | C38239 |
| INTRADUCTAL | Administration within the | ductI-DUCTAL | 123 | C38240 |
| | of a gland. | | | |
| INTRADUODENAL | Administration within the | I-DUOD | 047 | C38241 |
| | duodenum. | | | |
| INTRADURAL | Administration within or | I-DURAL | 052 | C38242 |
| | beneath the dura. | | | |
| INTRAEPIDERMAL | Administration within the | I-EPIDERM | 127 | C38243 |
| | epidermis. | | | |
| INTRAESOPHAGEAL | Administration within the | I-ESO | 072 | C38245 |
| | esophagus. | | | |
| INTRAGASTRIC | Administration within the | I-GASTRIC | 046 | C38246 |
| | stomach. | | | |
| INTRAGINGIVAL | Administration within the | I-GINGIV | 307 | C38247 |
| | gingivae. | | | |
| INTRAILEAL | Administration within the | I-ILE | 365 | C38249 |
| | distal portion of the small | | | |
| | intestine, from the jejunum to | | | |
| | the cecum. | | | |
| INTRALESIONAL | Administration within or | I-LESION | 042 | C38250 |
| | introduced directly into a | | | |
| | localized lesion. | | | |
| INTRALUMINAL | Administration within the | I-LUMIN | 310 | C38251 |
| | lumen of a tube. | | | |
| INTRALYMPHATIC | Administration within the | I-LYMPHAT | 352 | C38252 |
| | lymph. | | | |
| INTRAMEDULLARY | Administration within the | I-MEDUL | 408 | C38253 |
| | marrow cavity of a bone. | | | |
| INTRAMENINGEAL | Administration within the | I-MENIN | 409 | C38254 |
| | meninges (the three | | | |
| | membranes that envelope the | | | |
| | brain and spinal cord). | | | |
| INTRAMUSCULAR | Administration within a | IM | 005 | C28161 |
| | muscle. | | | |
| INTRAOCULAR | Administration within the eye. | I-OCUL | 036 | C38255 |
| INTRAOVARIAN | Administration within the | I-OVAR | 354 | C38256 |
| | ovary. | | | |
| INTRAPERICARDIAL | Administration within the | I-PERICARD314 | | C38257 |
| | pericardium. | | | |
| INTRAPERITONEAL | Administration within the | I-PERITON | 004 | C38258 |
| | peritoneal cavity. | | | |
| INTRAPLEURAL | Administration within the | I-PLEURAL | 043 | C38259 |
| | pleura. | | | |
| INTRAPROSTATIC | Administration within the | I-PROSTAT | 061 | C38260 |
| | prostate gland. | | | |
| INTRAPULMONARY | Administration within the | I-PULMON | 414 | C38261 |
| | lungs or its bronchi. | | | |
| INTRASINAL | Administration within the | I-SINAL | 010 | C38262 |
| | nasal or periorbital sinuses. | | | |
| INTRASPINAL | Administration within the | I-SPINAL | 022 | C38263 |
| | vertebral column. | | | |
| INTRASYNOVIAL | Administration within the | I-SYNOV | 019 | C38264 |
| | synovial cavity of a joint. | | | |
| INTRATENDINOUS | Administration within a | I-TENDIN | 049 | C38265 |
| | tendon. | | | |
| INTRATESTICULAR | Administration within the | I-TESTIC | 110 | C38266 |
| | testicle. | | | |
| INTRATHECAL | Administration within the | IT | 103 | C38267 |
| | cerebrospinal fluid at any | | | |
| | level of the cerebrospinal axis, | | | |
| | including injection into the | | | |
| | cerebral ventricles. | | | |
| INTRATHORACIC | Administration within the | I-THORAC | 006 | C38207 |
| | thorax (internal to the ribs); | | | |
| | synonymous with the term | | | |
| | endothoracic. | | | |
| INTRATUBULAR | Administration within the | I-TUBUL | 353 | C38268 |
| | tubules of an organ. | | | |
| INTRATUMOR | Administration within a | I-TUMOR | 020 | C38269 |
| | tumor. | | | |
| INTRATYMPANIC | Administration within the | I-TYMPAN | 366 | C38270 |
| | Auris media. | | | |
| INTRAUTERINE | Administration within the | I-UTER | 028 | C38272 |
| | uterus. | | | |
| INTRAVASCULAR | Administration within a vessel | I-VASC | 021 | C38273 |
| | or vessels. | | | |
| INTRAVENOUS | Administration within or into | IV | 002 | C38276 |
| | a vein or veins. | | | |
| INTRAVENOUS BOLUS | Administration within or into | IV BOLUS | 138 | C38274 |
| | a vein or veins all at once. | | | |
| INTRAVENOUS DRIP | Administration within or into | IV DRIP | 137 | C38279 |
| | a vein or veins over a | | | |
| | sustained period of time. | | | |
| INTRA VENTRICULAR | Administration within a | I-VENTRIC | 048 | C38277 |
| | ventricle. | | | |
| INTRAVESICAL | Administration within the | I-VESIC | 128 | C38278 |
| | bladder. | | | |
| INTRA VITREAL | Administration within the | I-VITRE | 311 | C38280 |
| | vitreous body of the eye. | | | |
| IONTOPHORESIS | Administration by means of | ION | 055 | C38203 |
| | an electric current where ions | | | |
| | of soluble salts migrate into | | | |
| | the tissues of the body. | | | |
| IRRIGATION | Administration to bathe or | IRRIG | 032 | C38281 |
| | flush open wounds or body | | | |
| | cavities. | | | |
| LARYNGEAL | Administration directly upon | LARYN | 364 | C38282 |
| | the larynx. | | | |
| NASAL | Administration to the nose; | NASAL | 014 | C38284 |
| | administered by way of the | | | |
| | nose. | | | |
| NASOGASTRIC | Administration through the | NG | 071 | C38285 |
| | nose and into the stomach, | | | |
| | usually by means of a tube. | | | |
| NOT APPLICABLE | Routes of administration are | NA | 312 | C48623 |
| | not applicable. | | | |
| OCCLUSIVE DRESSING | Administration by the topical | OCCLUS | 134 | C38286 |
| TECHNIQUE | route which is then covered by | | | |
| | a dressing which occludes the | | | |
| | area. | | | |
| OPHTHALMIC | Administration to the external | OPHTHALM | 012 | C38287 |
| | eye. | | | |
| ORAL | Administration to or by way | ORAL | 001 | C38288 |
| | of the mouth. | | | |
| OROPHARYNGEAL | Administration directly to the | ORO | 410 | C38289 |
| | mouth and pharynx. | | | |
| OTHER | Administration is different | OTHER | 135 | C38290 |
| | from others on this list. | | | |
| PARENTERAL | Administration by injection, | PAREN | 411 | C38291 |
| | infusion, or implantation. | | | |
| PERCUTANEOUS | Administration through the | PERCUT | 113 | C38676 |
| | skin. | | | |
| PERIARTICULAR | Administration around a joint. | P-ARTIC | 045 | C38292 |
| PERIDURAL | Administration to the outside | P-DURAL | 050 | C38677 |
| | of the dura mater of the spinal | | | |
| | cord. | | | |
| PERINEURAL | Administration surrounding a | P-NEURAL | 412 | C38293 |
| | nerve or nerves. | | | |
| PERIODONTAL | Administration around a tooth. | P-ODONT | 040 | C38294 |
| RECTAL | Administration to the rectum. | RECTAL | 016 | C38295 |
| RESPIRATORY | Administration within the | RESPIR | 136 | C38216 |
| (INHALATION) | respiratory tract by inhaling | | | |
| | orally or nasally for local or | | | |
| | systemic effect. | | | |
| RETROBULBAR | Administration behind the | RETRO | 034 | C38296 |
| | pons or behind the eyeball. | | | |
| SOFT TISSUE | Administration into any soft | SOFT TIS | 109 | C38198 |
| | tissue. | | | |
| SUBARACHNOID | Administration beneath the | S-ARACH | 066 | C38297 |
| | arachnoid. | | | |
| SUBCONJUNCTIVAL | Administration beneath the | S-CONJUNC | 096 | C38298 |
| | conjunctiva. | | | |
| SUBCUTANEOUS | Administration beneath the | SC | 003 | C38299 |
| | skin; | | | |
| | hypodermic. Synonymous | | | |
| | with the term SUBDERMAL. | | | |
| SUBLINGUAL | Administration beneath the | SL | 024 | C38300 |
| | tongue. | | | |
| SUBMUCOSAL | Administration beneath the | S-MUCOS | 053 | C38301 |
| | mucous membrane. | | | |
| TOPICAL | Administration to a particular | \|TOPIC | 011 | C38304 |
| | spot on the outer surface of | | | |
| | the body. The E2B term | | | |
| | TRANSMAMMARY is a | | | |
| | subset of the term TOPICAL. | | | |
| TRANSDERMAL | Administration through the | T-DERMAL | 358 | C38305 |
| | dermal layer of the skin to the | | | |
| | systemic circulation by | | | |
| | diffusion. | | | |
| TRANSMUCOSAL | Administration across the | T-MUCOS | 122 | C38283 |
| | mucosa. | | | |
| TRANSPLACENTAL | Administration through or | T-PLACENT | 415 | C38307 |
| | across the placenta. | | | |
| TRANSTRACHEAL | Administration through the | T-TRACHE | 355 | C38308 |
| | wall of the trachea. | | | |
| TRANSTYMPANIC | Administration across or | T-TYMPAN | 124 | C38309 |
| | through the tympanic cavity. | | | |
| UNASSIGNED | Route of administration has | UNAS | 400 | C38310 |
| | not yet been assigned. | | | |
| UNKNOWN | Route of administration is | UNKNOWN | \|139 | C38311 |
| | unknown. | | | |
| URETERAL | Administration into the ureter. | URETER | 112 | C38312 |
| URETHRAL | Administration into the | URETH | 017 | C38271 |
| | urethra. | | | |
| VAGINAL | Administration into the | VAGIN | 015 | C38313 |
| | vagina. | | | |

In some embodiments, the route of administration may be oral, intranasal, inhalation, intravenous, sublingual, topical, injectable and/or transdermal.

The pharmaceutical compositions of the present invention comprising the NMDA receptor antagonist and/or metal channel activator may also include other pharmaceutically acceptable carriers and/or excipients such as binders, lubricants, diluents, coatings, disintegrants, barrier layer components, glidants, coloring agents, solubility enhancers, gelling agents, fillers, proteins, co-factors, emulsifiers, solubilizing agents, suspending agents and mixtures thereof. A skilled artisan in the art would know what other pharmaceutically acceptable carriers and/or excipients could be included in the formulations according to the invention. The choice of excipients would depend on the characteristics of the compositions and on the nature of other pharmacologically active compounds in the formulation.

The formulator will understand that excipients are used primarily to serve in delivering a safe, stable, and functional pharmaceutical. Excipients may also serve as part of the overall vehicle for delivery. Excipients may also be used to achieve effective absorption by the recipient of the active ingredient. An excipient may fill a role as simple and direct as being an inert filler, or an excipient as used herein may be part of a pH stabilizing system or coating. Excipients herein may also serve an antimicrobial function or improve pharmaceutical composition characteristics such as lubricity, flowability, disintegration and taste.

Pharmaceutical compositions may comprise one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Pharmaceutically acceptable carriers are biologically acceptable and compatible with the other ingredients in the formulation. Appropriate excipients are known to those skilled in the art (see Handbook of Pharmaceutical Excipients, fifth edition, 2005 edited by Rowe et al., McGraw Hill) and have been utilized to yield a novel sublingual formulation with unexpected properties. Pharmaceutical compositions herein are not limited to a single active ingredient, and supplementary active ingredients can also be incorporated.

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose; other carriers such as, polyvinylpyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinylpyrrolidone, agar, or alginic acid or a salt thereof such as sodium alginate. The compositions may be prepared by any of the methods of pharmacy but all methods include the step of bringing into association one or more chemical agents as described above with the carrier which constitutes one or more necessary ingredients. In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes, and the like. NMDA receptor antagonists, metal channel activator, and/or the pharmaceutically acceptable salts thereof can be formulated using pharmaceutically acceptable carriers well known in the art into dosages suitable for sublingual, intranasal or buccal administration. Such carriers enable the NMDA receptor antagonists and/or metal channel activator to be formulated in dosage forms such as tablets, powders, pills, capsules, liquids, gels, films, syrups, slurries, suspensions, and the like.

The NMDA receptor antagonist and/or metal channel activator compounds disclosed can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. Oral formulations containing a compound disclosed can be any conventionally used oral form, including but not limited to tablets, capsules, buccal forms, troches, lozenges and oral liquids, suspensions, or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato, or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidone, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, ion exchange resins, benzyl alcohol, eucalyptol, gelatin, limonene, mannitol, menthol, menthone, methyl acetate, sucralose, and vanillin. Surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecyl sulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions for oral, parenteral (such as intravenous, intramuscular, or other injections), or inhalation administration including but not limited to suspensions, emulsions, syrups, and elixirs. An NMDA receptor antagonist and/or metal channel activator compound of the present teachings can be dissolved or suspended in a pharmaceutically acceptable liquid carrier such as water, an organic solvent, or a mixture of both, or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal, or subcutaneous injection. Sterile injectable solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be further sub-divided to contain appropriate quantities of the compound. The unit dosage forms can be packaged compositions, for example, packeted powders, vials, ampoules, prefilled syringes, or sachets containing liquids. Alternatively, the unit dosage form can be a capsule or tablet itself, or it can be the appropriate number of any such compositions in package form. Such doses can be administered in any manner useful in directing the compound(s) to the recipient's bloodstream, including orally, via implants, parenterally (including intravenous, intraperitoneal, and subcutaneous injections), rectally, vaginally, and transdermally.

When administered for the treatment or inhibition of a particular disease state or disorder, it is understood that an effective dosage can vary depending upon the particular compound utilized, the pharmaceutical composition formulated, the mode of administration, physical factors related to the individual being treated, and severity of the condition being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition, stage of said condition, and the characteristics of the patient including their size, age and response pattern to the compound utilized.

In some cases, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can be sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

### Pharmacokinetics and Dosing

In some embodiments, a method of treating a neurological disease may be characterized by one or more pharmacokinetic parameters such as AUC, Cₘₐₓ, Tₘₐₓ, and others known and understood to persons of skill in the art. The term “pharmacokinetic” (PK) as used herein is used in its ordinary sense to mean the pharmacokinetic aspects of drug delivery. By definition pharmacokinetics (PK) is the study of how an organism affect a drug, e.g., how, and how fast it metabolizes the drug. The pharmacokinetics typically vary based upon the dosage amount of one or more of the disclosed NMDA receptor antagonists and/or metal channel activators. The pharmacokinetics may or may not vary as a function of administration route, and, if any, the binders, excipients, and dilutants in the pharmaceutical composition.

In some embodiments, a method of treating a neurological disease may be characterized by an AUC for a NMDA receptor antagonist and/or metal channel activator compound according to Formulas 1-180, Formulas 200-260, compounds disclosed in the “further embodiments section” under the “METAL CHANNEL ACTIVATOR” subheading, and/or pharmaceutically acceptable salts thereof. In some embodiments, the AUC₀₋ₜ and/or AUC_{0-inf} (collectively referred to in the alternative as, simply, AUC) may be from about 80-125% of a given AUC value. In some embodiments, the AUC may be within 80-125% of about 25, 50, 100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 hr*ng/ml. In some embodiments, a systemic treatment may have a larger AUC than a localized (such as topical or subdermal) treatment.

In some embodiments, a method of treating a neurological disease may be characterized by a Cₘₐₓ for a NMDA receptor antagonist and/or metal channel activator compound according to Formulas 1-180, Formulas 200-260, compounds disclosed in the “further embodiments section” under the “METAL CHANNEL ACTIVATOR” subheading, and/or pharmaceutically acceptable salts thereof. In some embodiments, the Cₘₐₓ may be from about 80-125% of a given Cₘₐₓ value. In some embodiments, the Cₘₐₓ may be within 80-125% of about 1, 2, 3, 4, 5, 8, 10, 20, 35, 50, 80,100, 150, 200, 250, 300, 400, 500, 600, 700, 800, 900, 1000, 1300, 1600, 1900, 2500, 3500, 5000, 7500, 10,000, 15,000, 20,000, 30,000, 50,000, and/or 100,000 ng/mL. In some embodiments, a systemic treatment may have a larger Cₘₐₓ than a localized (such as topical or subdermal) treatment.

In some embodiments, a ratio Cₘₐₓ/AUC may be used to characterize a method of treating a neurological disease wherein one or more of a NMDA receptor antagonist and/or metal channel activator compound according to Formulas 1-180, Formulas 200-260, compounds disclosed in the “further embodiments section” under the “METAL CHANNEL ACTIVATOR” subheading, and/or pharmaceutically acceptable salts thereof are administered to a subject. In some embodiments, the Cₘₐₓ/AUC ratio may be from about 80-125% of a given Cₘₐₓ/AUC ratio. In some embodiments, the Cₘₐₓ/AUC ratio may be from about 80-125% of about 0.01, 0.03, 0.05, 0.08, 0.1, 0.13, 0.17, 0.2, 0.25, 0.3, 0.35, 0.4, 0.45, 0.5, 0.6, 0.7, 0.8, and 0.9.

In some embodiments, the Tₘₐₓ may range from about 0.1-16 hours, or from about 0.3-8 hours, or from about 0.5-4 hours, or from 0.5-2 hours, or from about 1-2 hours. In some embodiments, the route of administration, which may be any route described herein or known to a person of skill in the art, may affect the Tₘₐₓ of a compound according to Formulas 1-180, Formulas 200-260, compounds disclosed in the “further embodiments section” under the “METAL CHANNEL ACTIVATOR” subheading, and/or pharmaceutically acceptable salts thereof. Excipients or other pharmaceutically acceptable carriers in pharmaceutical compositions of one or more compounds according to Formulas 1-180, Formulas 200-238, compounds disclosed in the “further embodiments section” under the “METAL CHANNEL ACTIVATOR” subheading, and/or pharmaceutically acceptable salts thereof may alter the Tₘₐₓ value by making it larger or smaller than a pharmaceutical composition having the excipient or carrier excluded.

Solid compositions are normally formulated in dosage units providing from about 0.1 to about 1000 mg of the active ingredient per dose. Some examples of solid dosage units are 0.1 mg, 1 mg, 10 mg, 100 mg, 500 mg, and 1000 mg. Liquid compositions are generally in a unit dosage range of 0.1-100 mg/mL. Some examples of liquid dosage units are 0.1 mg/mL, 1 mg/mL, 10 mg/mL, 25 mg/mL, 50 mg/mL, and 100 mg/mL.

In some embodiments, a dose is daily. In some embodiments, a dose is twice daily. In some embodiments, a does is one, two, three, four, or five times daily. In some embodiments, a dose is once every other day, once every second day, once every third day, once every fourth day, once every fifth day, once every sixth day, weekly, bi-weekly, or monthly. Typically, a dose may be from 0.01-100 mg/kg body weight, or from 0.05-50 mg/kg body weight, or from 0.1-10 mg/kg body weight, or from 0.15-5 mg/kg body weight, or from 0.2-2 mg/kg body weight, or from 0.5-1.5 mg/kg body weight, or from 1-1.5 mg/kg body weight. In some embodiments, the dosing regimen may comprise one or more optional loading doses and a subsequent maintenance dose regimen. A loading dose may be larger than the doses given in a subsequent maintenance dose regimen.

In some embodiments, the dosage is adjusted based upon neurological disease symptoms observed in the patient. A symptom-dependent regimen may have a larger treatment dose while symptoms are present and a smaller preventative dose while symptoms are lessened, in remission, controlled, etc. In some embodiments, a treatment dose may comprise a unit dosage of about 10 mg, 20 mg, 30 mg, 40 mg, 50 mg, 60 mg, 70 mg, 75 mg, 80 mg, 100 mg, 120 mg, 140 mg, 160 mg, 180 mg, or 200 mg of an NMDA receptor antagonist according to any one of Formulas 1-180, and/or a metal channel activator according to any one of Formulas 200-260, or a compounds disclosed in the “further embodiments section” under the “METAL CHANNEL ACTIVATOR” subheading, and/or pharmaceutically acceptable salts thereof. In some embodiments, a preventative dose may comprise a unit dosage of about 1 mg, 2 mg, 5 mg, 10 mg, 20 mg, 30 mg, 40 mg, 50 mg, 60 mg, 70 mg, 75 mg, 80 mg, 100 mg, 120 mg, or 140 mg of a NMDA receptor antagonist according to any one of Formulas 200-260, and/or a metal channel activator according to any one of Formulas 1-180, or a compounds disclosed in the “further embodiments section” under the “METAL CHANNEL ACTIVATOR” subheading, and/or pharmaceutically acceptable salts thereof. In some embodiments, the preventative dose is an amount that is about 7/8, 3/4, 5/8, 1/2, 3/8, 1/4, or about ⅛ of the amount of the treatment dose. In some embodiments, if a patient receiving a preventative dosing regimen experiences recurrence or onset of symptoms, a treatment dosing regimen may replace the preventative dosing regimen until symptoms have lessened or subsided. Each of the treatment and/or preventative doses may be adjusted during the regimen based upon the severity of neurological disorder symptoms.

1. A pharmaceutical composition comprising a metal channel activator and an NMDA receptor antagonist.

2. The pharmaceutical composition of claim 1, wherein the metal channel activator is a potassium channel activator.

3. The pharmaceutical composition of claim 2, wherein the potassium channel activator is a Kv7 channel activator.

4. The pharmaceutical composition of claim 3, wherein the Kv7 channel activator is a Kv7.1 channel activator, a Kv7.2 channel activator, a Kv7.3 channel activator, a Kv7.4 channel activator, a Kv7.5 channel activator, or any combination thereof.

5. The pharmaceutical composition of claim 3, wherein the Kv7 channel activator is a Kv7.2/7.3 channel activator.

6. The pharmaceutical composition of claim 1, wherein the metal channel activator is selected from one or more compounds according to any one or more of formulas 1 to 171.

7. The pharmaceutical composition of claim 1, wherein the NMDA receptor antagonist is selected from one or more compounds according to any one or more of formulas 200 to 260.

8. The pharmaceutical composition of claim 1, wherein the NMDA receptor antagonist is selected from one or more compounds according to any one or more of formulas 200 to 260, and wherein the metal channel activator is selected from one or more of the disclosed compounds according to any one or more of formulas 1 to 171.

9. The pharmaceutical composition of claim 1, wherein the metal channel activator is

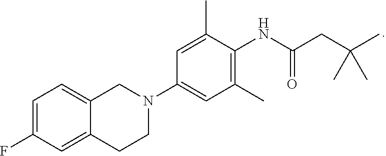 [SMILES: Cc1cc(N2CCc3cc(F)ccc3C2)cc(C)c1NC(=O)CC(C)(C)C]

10. The pharmaceutical composition of claim 1, wherein the metal channel activator is

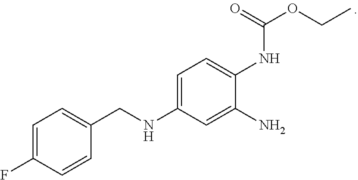 [SMILES: CCOC(=O)Nc1ccc(NCc2ccc(F)cc2)cc1N]

11. The pharmaceutical composition of claim 1, wherein the NMDA receptor antagonist is ketamine, lanicemine, dextromethorphan (DXM), phencyclidine (PCP), methoxetamine (MXE), or a pharmaceutically acceptable salt thereof.

12. The pharmaceutical composition of claim 1, wherein the NMDA receptor antagonist or a pharmaceutically acceptable salt thereof is in a form of a prodrug.

13. The pharmaceutical composition of claim 1, wherein the NMDA receptor antagonist is lanicemine or a pharmaceutically acceptable salt thereof.

14. The pharmaceutical composition of claim 13, wherein the lanicemine or a pharmaceutically acceptable salt thereof is in a form of a prodrug.

15. The pharmaceutical composition of claim 14, wherein the prodrug of lanicemine has the following structure: 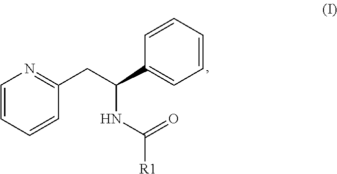
wherein R¹ is C₁₋₆ alkylC(O)O(C₁₋₆alkoxy), or 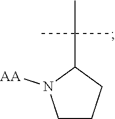
AA is a peptide bond-linked natural amino acid;
or a pharmaceutically acceptable salt thereof.

16. The pharmaceutical composition of claim 14, wherein the prodrug of lanicemine has the following structure: 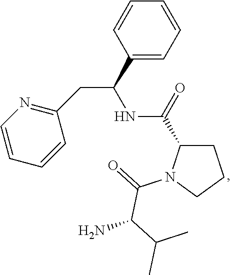 [SMILES: CC(C)[C@H](N)C(=O)N1CCC[C@H]1C(=O)N[C@@H](Cc1ccccn1)c1ccccc1]
or a pharmaceutically acceptable salt thereof.

17. A method of treating a depressive disorder, comprising administering a therapeutically effective amount of the pharmaceutical composition of claim 1 to a patient in need thereof.

18. The method of claim 17, wherein the depressive disorder is major depressive disorder (MDD), disruptive mood dysregulation disorder, persistent depressive disorder, bipolar spectrum disorder, postpartum depression, premenstrual dysphoric disorder (PMDD), seasonal affective disorder (SAD), atypical depression, treatment-resistant depression (TRD), depression associated with agitation or anxiety, adjustment disorder with depressed mood, prolonged depressive reaction, or a combination thereof.

19. A method of treating a neurological or neurodegenerative disorder, comprising administering a therapeutically effective amount of the pharmaceutical composition of claim 1 to a patient in need thereof.

20. The method of claim 19, wherein neurological or neurodegenerative disorder is amyotrophic lateral sclerosis, bipolar disorder, treatment resistant and major depression, general anxiety disorder, panic disorder, social anxiety, mood disorders, cognitive disorders, dementia, agitation, apathy, psychoses, post-traumatic stress disorders, irritability, disinhibition, learning disorders, memory loss, personality disorders, bipolar disorders, Rett syndrome, eating disorders, conduct disorder, neurodegenerative disorders, pain disorders, supranuclear palsy, frontotemporal dementia, frontotemporal lobar degeneration, delirium, Alzheimer's disease, mild cognitive impairment, mild cognitive impairment due to Alzheimer's disease, drug addiction, tinnitus, mental retardation, obsessive-compulsive disorder, spinal muscular atrophy, radiation therapy, multiple sclerosis, chronic cerebellar ataxia, cervical spondylotic myelopathy, spinal cord injury, hereditary cerebellar ataxia, Tourette syndrome, autism spectrum disorder, schizophrenia, fragile X syndrome, Parkinson's Disease, Huntington's disease, or any combination thereof.

21. A method of treating a pain disorder, comprising administering a therapeutically effective amount of the pharmaceutical composition of claim 1 to a patient in need thereof.

22. The method of claim 21, wherein the pain disorder is acute pain, chronic pain, neuropathic pain, nociceptive pain, and/or radicular pain.

23. The method of claim 21, wherein the pain disorder is migraine, inflammatory pain, persistent pain, cancer pain, and/or postoperative pain.

24. A kit for treating a patient afflicted with a disorder in which a metal channel activator is clinically relevant, said kit including:
(a) the metal channel activator; and
(b) instructions for administering said metal channel activator in combination with an NMDA receptor antagonist by the method of claim 17.

25. A kit for treating a patient afflicted with a disorder in which an NMDA receptor antagonist is clinically relevant, said kit including:
(a) the NMDA receptor antagonist; and
(b) instructions for administering said NMDA receptor antagonist with a metal channel activator by the method of claim 17.


SEQUENCE LISTING (18 sequences)

SEQ ID NO: 1 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, Rattus norvegicus
  REGION 24..38: note = CDR1 is KSSQSLVHTDGKTYLH
  REGION 54..61: note = CDR2 is QVSNLGS
  REGION 94..102: note = CD3 is VQATHFPLT
DVVLTQTPPPLSVAIGQSVSISCKSSQSLVHTDGKTYLHWLLQSPGQSPKLLIYQVSNLGSGVPDRFSGTGSQKDFTLKISRVEAEDLGVYYCVQATHFPLTFGIGTKLELQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
  VARIANT 234..235: note = LALA = L234A/L235A
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
  VARIANT 234..235: note = LALA = L234A/L235A
  VARIANT 329: note = LALA/PA = L234A/L235/P329A
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
  VARIANT 234..235: note = LALA = L234A/L235A
  VARIANT 329: note = LALA/PA = L234A/L235/P329A
  VARIANT 428: note = LS = M428L/N434S
  VARIANT 434: note = LS = M428L/N434S
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 6 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
  VARIANT 234..235: note = LALA = L234A/L235A
  VARIANT 329: note = LALA/PA = L234A/L235/P329A
  VARIANT 252: note = YTE = M252Y/S254T/T256E
  VARIANT 254: note = YTE = M252Y/S254T/T256E
  VARIANT 256: note = YTE = M252Y/S254T/T256E
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
  VARIANT 234..235: note = LALA = L234A/L235A
  VARIANT 428: note = LS = M428L/N434S
  VARIANT 434: note = LS = M428L/N434S
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 8 - protein, 446 aa, Rattus norvegicus
  REGION 31..36: note = CDR1 is SNYWG
  REGION 49..65: note = CDR2 is YITYSGGTYYNPSLKS
  REGION 98..104: note = CDR3 is WGDWYFDF
  VARIANT 234..235: note = LALA = L234A/L235A
  VARIANT 252: note = M252Y/S254T/T256E
  VARIANT 254: note = M252Y/S254T/T256E
  VARIANT 256: note = M252Y/S254T/T256E
ELQLQESGPGLVKPSQSLSLTCSVTGYSIRSNYWGWIRKFPGNKMEWMGYITYSGGTYYNPSLKSRISITRDTSKNQFFLQLTSVTTEDTATYYCTRWGDWYFDFWGPGTKVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 5 aa, Rattus norvegicus
SNYWG

SEQ ID NO: 10 - protein, 16 aa, Rattus norvegicus
YITYSGGTYYNPSLKS

SEQ ID NO: 11 - protein, 8 aa, Rattus norvegicus
WGDWYFDF

SEQ ID NO: 12 - protein, 16 aa, Rattus norvegicus
KSSQSLVHTDGKTYLH

SEQ ID NO: 13 - protein, 7 aa, Rattus norvegicus
QVSNLGS

SEQ ID NO: 14 - protein, 9 aa, Rattus norvegicus
VQATHFPLT

SEQ ID NO: 15 - protein, 5 aa, synthetic construct
LPETG

SEQ ID NO: 16 - protein, 330 aa, Homo sapiens
ASTKGPSVFPLAPSSKSTSGGTAALGCLVKDYFPBPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWINGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEATHNHYTQKSLSLSPGK

SEQ ID NO: 17 - protein, 326 aa, Homo sapiens
ASTKGPSVFPLAPCSRSTSESTAALGCLVKDYFPBPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREBQFNSTFRVVSVLTVLHQDWLNGKEYKCKVSNKGLPAPIEKTISKTKGOPREPQVYTLPPSREEMTKNQVSLTCLVKGFYPSDIAVEWESNGQPENNYKTTPPMLDSDGSFFLYSKLTVDKSRWQQGNVFSCSVMHEATHNHYTQKSLSLSPGK

SEQ ID NO: 18 - protein, 327 aa, Homo sapiens
ASTKGPSVFPLAPCSRSTSESTAALGCLVKDYFPBPVTVSWNSGALTSGVHTFPAVLQSSGLYSLSSVVTVPSSSLGTKTYTCNVDHKPSNTKVDKRVESKTGPPCPSCPAPEFLGGPSVFLFPPKPKDTLMISRTPEVTCVVVDVSQEDPEVQFNWYVDGVEVHNAKTKPREEQFNSTYRVVSVLTVLHQDWINGKEYKCKVSNKGLPSSIEKTISKAKGOPREPQVYTLPPSQEEMTKNQVSLTCLVKGFYPSDIAVEWESNGQPENNYKTTPPVLDSDGSFFLYSRLTVDKSRWQEGNVFSCSVMHEATHNHYTQKSLSLSLGK